(19) United States
(12) Patent Application Publication
(10) Pub. No.: US 2024/0083897 A1
(43) Pub. Date: Mar. 14, 2024

(54) GCN2 MODULATING COMPOUNDS AND USES THEREOF

(71) Applicant: HiberCell, Inc., New York, NY (US)

(72) Inventors: Savithiri Ramurthy, New York, NY (US); Mark J. Mulvihill, Sudbury, MA (US); Bradley Sherborne, Hoddesdon (GB); Benjamin Rahemtulla, Hoddesdon (GB); Eric P.A. Talbot, Hoddesdon (GB); Christopher G. Thomson, Hoddesdon (GB)

(21) Appl. No.: 18/262,493

(22) PCT Filed: Jan. 21, 2022
(86) PCT No.: PCT/US2022/013383

§ 371 Date: Jul. 21, 2023

Related U.S. Application Data
(60) Provisional application No. 63/140,314, filed on Jan. 22, 2021.

(51) Int. Cl. C07D 471/04; C07D 487/04; C07D 513/04; C07D 498/04

(52) U.S. Cl. CPC C07D 471/04; C07D 487/04; C07D 513/04; C07D 498/04

(57) ABSTRACT

Provided herein are compounds, compositions, and methods useful for modulating the activity of GCN2 and for treating related conditions, diseases, and disorders (e.g., cancer and neurodegenerative diseases).

### CROSS REFERENCE TO RELATED APPLICATION

This application claims priority to U.S. Provisional Application No. 63/140,314 filed Jan. 22, 2021, which is incorporated herein by reference in its entirety

### BACKGROUND OF THE INVENTION

Cancer continues to be a significant health problem despite the substantial research efforts and scientific advances reported in the literature for treating this disease. Some of the most frequently diagnosed cancers include prostate cancer, breast cancer, and lung cancer. Prostate cancer is the most common form of cancer in men. Breast cancer remains a leading cause of death in women. Current treatment options for these cancers are not effective for all patients and/or can have substantial adverse side effects. New therapies are needed to address this unmet need in cancer therapy.

General control nonderepressible kinase 2 (GCN2) is a serine/threonine protein kinase that phosphorylates the a subunit of eukaryotic initiation factor 2 (eIF2α) in response to amino acid deficiency (see, for example, Wek, R. C. et al. in Biochem. Soc. Trans. 2006, 34 (Pt 1), p. 7-11). Expression and activation of GCN2 have been shown to be elevated in human and mouse tumors, and reduction in the expression of GCN2 has been shown to inhibit tumor growth (see e.g., Ye, J. et al. in EMBO J. 2010, 29(12), p. 2082-2096). Tumors grow in an environment of amino acid deficiency which can be further depleted with chemotherapy inducing a dependence on autophagy which requires GCN2 activity. In addition, GCN2 mediates the induction of anergy in T cells in response to tryptophan depletion by indoleamine 2,3-dioxygenase (IDO) in the tumor microenvironment (Munn, D. H. et al in Immunity 2005, 22, p. 633-642) and is essential for the proliferative fitness of cytotoxic T cells in amino acid limiting environments (Van de Velde, L-A., et al. in Cell Reports 2016, 17, p. 2247-2258). Inhibition of GCN2 has been reported as a therapeutic approach for cancer therapy (see, e.g., Wei, C. et al. in Mol. Biol. Cell. 2015, 26(6), p. 1044-1057). Accordingly, compounds having modulatory activity towards GCN2 are needed as therapeutic agents for treating cancer, with additional applications in the treatment of neurodegenerative diseases and doxorubicin-induced cardiotoxicity.

### SUMMARY OF THE INVENTION

Provided herein are compounds and compositions for the modulation of GCN2 (e.g., the activation or inhibition of GCN2). In various embodiments, the compounds and compositions described herein are useful for the treatment of GCN2 mediated conditions, diseases, or disorders (e.g., cancers and neurodegenerative diseases).

In one aspect, provided herein is a compound represented by Formula (Ia)

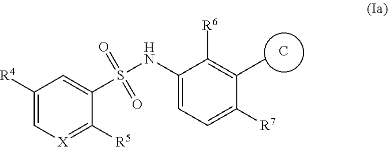

or a pharmaceutically acceptable salt thereof, wherein:
C is selected from 7-10 membered bicyclic heterocyclyl, 8 membered bicyclic heteroaryl, or 9-membered bicyclic heteroaryl selected from the group consisting of

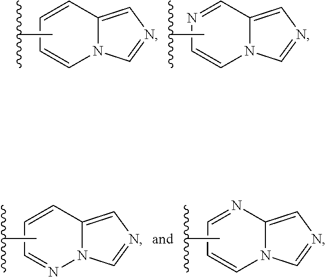

wherein the 7-10 membered bicyclic heterocyclyl, 8 membered bicyclic heteroaryl, or 9 membered bicyclic heteroaryl may be optionally substituted on one or more available carbons by one, two, three, or more substituents each independently selected from R³; wherein the 7-10 membered bicyclic heterocyclyl is partially unsaturated and contains at least two nitrogen atoms; wherein the 8 membered bicyclic heteroaryl contains at least two nitrogen items; wherein, if the 7-10 membered bicyclic heterocyclyl, 8 membered bicyclic heteroaryl, or 9 membered bicyclic heteroaryl contains a substitutable ring nitrogen atom, that ring nitrogen atom may be optionally substituted by C₁₋₆alkyl;
X is selected from the group consisting of CH, C(R⁸) and N;
R³ is independently, for each occurrence, selected from the group consisting of halogen, C₁₋₆alkyl, C₃₋₆cycloalkyl, cyano, C₁₋₆alkoxyl, hydroxyl, oxo, phenyl, —C(O)N(R^{A})(R^{B}), —N(R^{A})(R^{B}), 5-10 membered heterocyclyl, and 5-10 membered heteroaryl, wherein the C₁₋₆alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl;
R⁴ is selected from the group consisting of halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{4a};
R⁵ is selected from the group consisting of hydrogen, halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{5a};
R⁶ is selected from the group consisting of halogen, C₁₋₆alkyl, cyano, C₁₋₆alkoxyl, and C₃₋₆cycloalkyl, wherein the C₁₋₆alkyl and C₁₋₆cycloalkyl may be optionally substituted on one or more available carbons by one, two, three, or more substituents each independently selected from R^{6a};
R⁷ is selected from the group consisting of fluoro, chloro, methyl, and cyano;
R⁸ is selected from is selected from the group consisting of halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{8a};
or when X is C(R⁸), R⁸ and R⁵ may optionally combine together with the atoms to which they are attached to form a 3-7 membered carbocyclyl, 3-7 membered heterocyclyl, or 5-10 membered heteroaryl, wherein the 3-7 membered carbocyclyl, 3-7 membered heterocyclyl, or 5-10 membered heteroaryl may be optionally substituted on one or more available carbons by one, two, three, or more hydroxyl substituents; wherein, if the 3-7 membered carbocyclyl, 3-7 membered heterocyclyl, or 5-10 membered heteroaryl contains a substitutable ring nitrogen atom, that ring nitrogen atom may be optionally substituted by C₁₋₆alkyl;
R^{A} is independently, for each occurrence, hydrogen or C₁₋₆alkyl;
R^{B} is independently, for each occurrence, selected from the group consisting of hydrogen, C₁₋₆alkyl, C₃₋₆cycloalkyl, phenyl, 5-6 membered heterocyclyl, and 5-6 membered heteroaryl, wherein the C₁₋₆alkyl or phenyl may be optionally substituted on one or more available carbons by one, two, three, or more substituents each independently selected from R^{e}; and wherein, if the 5-6 membered heteroaryl or 5-6 membered heterocyclyl contains a substitutable ring nitrogen atom, that ring nitrogen atom may be optionally substituted by C₁₋₆alkyl;
R^{3a} is independently, for each occurrence, selected from the group consisting of halogen, cyano, hydroxyl, C₁₋₆alkyl, phenyl, C₃₋₆cycloalkyl, 5-6 membered heterocyclyl, and —CO₂(C₁₋₆alkyl), wherein the C₁₋₆alkyl may be optionally substituted on one or more available carbons by one, two, three, or more substituents each independently selected from halogen, hydroxyl, and phenyl; and wherein, if the 5-6 membered heterocyclyl contains a substitutable ring nitrogen atom, that ring nitrogen atom may be optionally substituted by C₁₋₆alkyl;
R^{4a} is independently, for each occurrence, selected from the group consisting of halogen, hydroxyl, and phenyl;
R⁵, is independently, for each occurrence, selected from the group consisting of halogen, hydroxyl, and phenyl;
R^{6a} is independently, for each occurrence, halogen;
R^{8a} is independently, for each occurrence, selected from the group consisting of halogen, hydroxyl, and phenyl;
R^{e} is independently, for each occurrence, selected from the group consisting of halogen, hydroxyl, C₁₋₆alkyl, 3-6 membered cycloalkyl, 5-6 membered heterocyclyl, —N(R^{C})(R^{D}), and —S(O)₂C₁₋₆alkyl, wherein C₁₋₆alkyl or 5-6 membered heterocyclyl may be optionally substituted on one or more available carbons by one, two, three, or more substituents each independently selected from oxo and —N(R^{C})(R^{D}); and
R^{C} and R^{D} are each independently selected from hydrogen and C₁₋₆alkyl; wherein the compound of Formula (I-1) is not

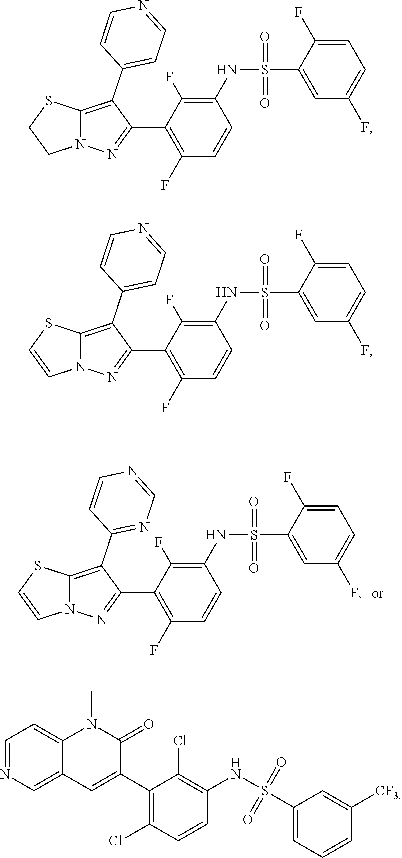 [SMILES: O=S(=O)(Nc1ccc(F)c(-c2nn3c(c2-c2ccncc2)SCC3)c1F)c1cc(F)ccc1F ; O=S(=O)(Nc1ccc(F)c(-c2nn3ccsc3c2-c2ccncc2)c1F)c1cc(F)ccc1F ; O=S(=O)(Nc1ccc(F)c(-c2nn3ccsc3c2-c2ccncn2)c1F)c1cc(F)ccc1F ; Cn1c(=O)c(-c2c(Cl)ccc(NS(=O)(=O)c3cccc(C(F)(F)F)c3)c2Cl)cc2cnccc21]

In another aspect, provided herein is a compound represented by Formula (Ia)

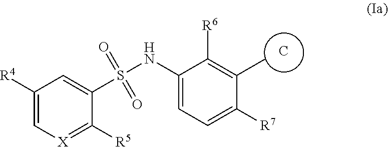

or a pharmaceutically acceptable salt thereof, wherein:
C is selected from 9-membered bicyclic heterocyclyl and 8-9 membered bicyclic heteroaryl selected from the group consisting of

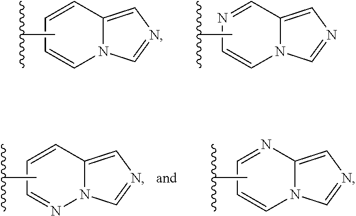

wherein the 9 membered bicyclic heterocyclyl or 8-9 membered bicyclic heteroaryl may be optionally substituted on one or more available carbons by one, two, three, or more substituents each independently selected from R³; wherein the 9 membered bicyclic heterocyclyl is partially unsaturated and contains at least two nitrogen atoms; wherein, if the 9 membered bicyclic heterocyclyl or 8-9 membered bicyclic heteroaryl contains a substitutable ring nitrogen atom, that ring nitrogen atom may be optionally substituted by C₁₋₆alkyl;
X is selected from the group consisting of CH, C(R⁸) and N;
R³ is independently, for each occurrence, selected from the group consisting of halogen, C₁₋₆alkyl, C₃₋₆cycloalkyl, cyano, C₁₋₆alkoxyl, hydroxyl, oxo, phenyl, —C(O)N(R^{A})(R^{B}), —N(R^{A})(R^{B}), 5-10 membered heterocyclyl, and 5-10 membered heteroaryl, wherein the C₁₋₆ alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl;
R⁴ is selected from the group consisting of halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{4a};
R⁵ is selected from the group consisting of halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{5a};
R⁶ is selected from the group consisting of halogen, C₁₋₆alkyl, cyano, C₁₋₆alkoxyl, and C₃₋₆cycloalkyl, wherein the C₁₋₆alkyl and C₁₋₆cycloalkyl may be optionally substituted on one or more available carbons by one, two, three, or more substituents each independently selected from R^{6a};
R⁷ is selected from the group consisting of fluoro, chloro, methyl, and cyano; R⁸ is selected from is selected from the group consisting of halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{8a};
or when X is C(R⁸), R⁸ and R⁵ may optionally combine together with the atoms to which they are attached to form a 3-7 membered carbocyclyl, 3-7 membered heterocyclyl, or 5-10 membered heteroaryl, wherein the 3-7 membered carbocyclyl, 3-7 membered heterocyclyl, or 5-10 membered heteroaryl may be optionally substituted on one or more available carbons by one, two, three, or more hydroxyl substituents; wherein, if the 3-7 membered carbocyclyl, 3-7 membered heterocyclyl, or 5-10 membered heteroaryl contains a substitutable ring nitrogen atom, that ring nitrogen atom may be optionally substituted by C₁₋₆alkyl;
R^{A} is independently, for each occurrence, hydrogen or C₁₋₆alkyl;
R^{B} is independently, for each occurrence, selected from the group consisting of hydrogen, C₁₋₆alkyl, C₃₋₆cycloalkyl, phenyl, 5-6 membered heterocyclyl, and 5-6 membered heteroaryl, wherein the C₁₋₆alkyl or phenyl may be optionally substituted on one or more available carbons by one, two, three, or more substituents each independently selected from R^{e}; and wherein, if the 5-6 membered heteroaryl or 5-6 membered heterocyclyl contains a substitutable ring nitrogen atom, that ring nitrogen atom may be optionally substituted by C₁₋₆alkyl;
R^{3a} is independently, for each occurrence, selected from the group consisting of halogen, cyano, hydroxyl, C₁₋₆alkyl, phenyl, C₃₋₆cycloalkyl, 5-6 membered heterocyclyl, and —CO₂(C₁₋₆ alkyl), wherein the C₁₋₆alkyl may be optionally substituted on one or more available carbons by one, two, three, or more substituents each independently selected from halogen, hydroxyl, and phenyl; and wherein, if the 5-6 membered heterocyclyl contains a substitutable ring nitrogen atom, that ring nitrogen atom may be optionally substituted by C₁₋₆alkyl;
R^{4a} is independently, for each occurrence, selected from the group consisting of halogen, hydroxyl, and phenyl;
R^{5a} is independently, for each occurrence, selected from the group consisting of halogen, hydroxyl, and phenyl;
R^{6a} is independently, for each occurrence, halogen;
R^{8a} is independently, for each occurrence, selected from the group consisting of halogen, hydroxyl, and phenyl;
R^{e} is independently, for each occurrence, selected from the group consisting of halogen, hydroxyl, C₁₋₆alkyl, 3-6 membered cycloalkyl, 5-6 membered heterocyclyl, —N(R^{C})(R^{D}), and —S(O)₂C₁₋₆alkyl, wherein C₁₋₆alkyl or 5-6 membered heterocyclyl may be optionally substituted on one or more available carbons by one, two, three, or more substituents each independently selected from oxo and —N(R^{C})(R^{D}); and
R^{C} and R^{D} are each independently selected from hydrogen and C₁₋₆alkyl.

In another aspect, provided herein is a compound represented by Formula (Ia)

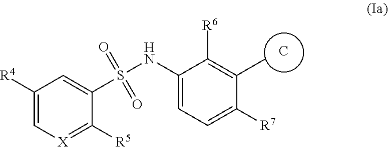

or a pharmaceutically acceptable salt thereof, wherein:
C is selected from 9-membered bicyclic heterocyclyl and 8-9 membered bicyclic heteroaryl selected from the group consisting of

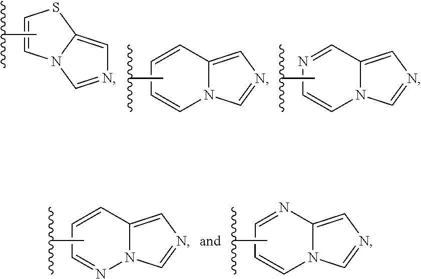

wherein the 9-membered bicyclic heterocyclyl or 8-9 membered bicyclic heteroaryl may be optionally substituted on one or more available carbons by one, two, three, or more substituents each independently selected from R³; wherein the 9 membered bicyclic heterocyclyl is partially unsaturated and contains at least two nitrogen atoms; wherein, if the 9 membered bicyclic heterocyclyl or 8-9 membered bicyclic heteroaryl contains a substitutable ring nitrogen atom, that ring nitrogen atom may be optionally substituted by C₁₋₆alkyl;
X is selected from the group consisting of CH, C(R⁸) and N;
R³ is independently, for each occurrence, selected from the group consisting of halogen, C₁₋₆alkyl, C₁₋₆alkoxyl, oxo, —C(O)N(R^{A})(R^{B}), —N(R^{A})(R^{B}), 5-10 membered heterocyclyl, and 5-10 membered heteroaryl, wherein the C₁₋₆alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl;
R⁴ is selected from the group consisting of halogen, C₁₋₆alkyl, and cyano, wherein the C₁₋₆alkyl may be optionally substituted on one or more available carbons by one, two, three, or more substituents each independently selected from R^{4a};
R⁵ is selected from the group consisting of C₁₋₆alkyl, cyano, hydroxyl, C₁₋₆alkoxyl, and —O—C₃₋₆cycloalkyl, wherein the C₁₋₆alkyl and C₁₋₆alkoxyl may be optionally substituted on one or more available carbons by one, two, three, or more substituents each independently selected from R^{5a};
R⁸ is cyano;
or when X is C(R⁸), R⁸ and R⁵ may optionally combine together with the atoms to which they are attached to form a 3-7 membered carbocyclyl, wherein the 3-7 membered carbocyclyl may be optionally substituted on one or more available carbons by one, two, three, or more hydroxyl substituents;
R⁶ is selected from the group consisting of hydrogen, halogen, C₁₋₆alkyl, and cyano, wherein the C₁₋₆alkyl may be optionally substituted on one or more available carbons by one, two, three, or more substituents each independently selected from R^{6a};
R⁷ is halogen;
R^{A} is hydrogen;
R^{B} is independently, for each occurrence, selected from the group consisting of hydrogen, C₁₋₆alkyl, phenyl, 5-6 membered heterocyclyl, and 5-6 membered heteroaryl, wherein the C₁₋₆alkyl or phenyl may be optionally substituted on one or more available carbons by one, two, three, or more substituents each independently selected from R^{e}; and wherein, if the 5-6 membered heteroaryl or 5-6 membered heterocyclyl contains a substitutable ring nitrogen atom, that ring nitrogen atom may be optionally substituted by C₁₋₆alkyl;
R^{3a} is independently, for each occurrence, selected from the group consisting of halogen, cyano, hydroxyl, C₁₋₆alkyl, phenyl, C₃₋₆cycloalkyl, 5-6 membered heterocyclyl, and —CO₂(C₁₋₆alkyl), wherein the C₁₋₆alkyl may be optionally substituted on one or more available carbons by one, two, three, or more substituents each independently selected from halogen, hydroxyl, and phenyl; and wherein, if the 5-6 membered heterocyclyl contains a substitutable ring nitrogen atom, that ring nitrogen atom may be optionally substituted by C₁₋₆alkyl;
R^{4a} is independently, for each occurrence, halogen;
R^{5a} is independently, for each occurrence, selected from halogen and phenyl;
R^{6a} is halogen;
R^{e} is independently, for each occurrence, selected from the group consisting of halogen, hydroxyl, C₁₋₆alkyl, 3-6 membered cycloalkyl, 5-6 membered heterocyclyl, —N(R^{C})(R^{D}), and —S(O)₂C₁₋₆alkyl, wherein the C₁₋₆alkyl or 5-6 membered heterocyclyl may be optionally substituted on one or more available carbons by one, two, three, or more substituents each independently selected from oxo and —N(R^{C})(R^{D}); and
R^{C} and R^{D} are each independently selected from hydrogen and C₁₋₆alkyl.

In another aspect, provided herein is a compound of selected from any compound set forth in Table 1, or a pharmaceutically acceptable salt thereof.

In another aspect, provided herein is a pharmaceutical composition comprising a compound of any embodiment and a pharmaceutically acceptable carrier.

In another aspect, provided herein is a method of treating cancer in a subject in need thereof, comprising administering to the subject a therapeutically effective amount of a compound of any embodiment.

In some embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, lymphoma, or any combination thereof.

In another aspect, provided herein is a method of treating a neurodegenerative disease in a subject in need thereof, comprising administering to the subject a therapeutically effective amount of a compound of any embodiment or a pharmaceutical composition of any embodiment. In some embodiments, the neurodegenerative disease is Alzheimer's disease, Parkinson's Disease, Huntington's Disease, amyotrophic lateral sclerosis, or spinocerebellar ataxia.

In another aspect, provided herein is a method of treating doxorubicin-induced cardiotoxicity in a subject in need thereof, comprising administering to the subject a therapeutically effective amount of a compound of any embodiment or a pharmaceutical composition of any embodiment.

In another aspect, provided herein is a method of modulating the activity of GCN2, comprising exposing GCN2 to an effective amount of a compound of any embodiment or a pharmaceutical composition of any embodiment to modulate the activity of said GCN2.

### DETAILED DESCRIPTION OF THE INVENTION

The invention provides GCN2-interacting compounds and related compounds, pharmaceutical compositions, and their use in the treatment of medical conditions, such as cancer, neurodegenerative diseases, and doxorubicin-induced cardiotoxicity, and in modulating (inhibiting/activating) GCN2 activity. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “—O-alkyl” etc.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. Exemplary alkylene groups include —CH₂—, —CH₂CH₂—, and —CH₂C(H)(CH₃)CH₂—. The term “—(C₀ alkylene)-” refers to a bond. Accordingly, the term “—(C₀₋₃ alkylene)-” encompasses a bond (i.e., C₀) and a —(C₁₋₃ alkylene) group.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In certain embodiments, a carbocyclyl group has 3 to 8 ring carbon atoms. In certain embodiments, a carbocyclyl group has 3 to 7 ring carbon atoms (“C₃₋₇ carbocycyl”). In certain embodiments, a carbocyclyl group has 3 to 6 ring carbon atoms (“C₃₋₆ carbocyclyl”). In certain embodiments, a carbocyclyl group has 5 to 10 ring carbon atoms (“C₅₋₁₀ carbocyclyl”). In certain embodiments, a carbocyclyl group has 5 to 10 ring carbon atoms (“C₇₋₁₀ carbocyclyl”). Exemplary C₃₋₆ carbocyclyl groups include, without limitation, cyclopropyl (C3), cyclobutyl (C4), cyclobutenyl (C4), cyclopentyl (C5), cyclopentenyl (C5), cyclohexyl (C6), cyclohexenyl (C6), cyclohexadienyl (C6), and the like. Exemplary C₃₋₈ carbocyclyl groups include, without limitation, the aforementioned C₃₋₆ carbocyclyl groups as well as cycloheptyl (C7), cycloheptenyl (C7), cycloheptadienyl (C7), cycloheptatrienyl (C7), cyclooctyl (C8), cyclooctenyl (C8), bicyclo[2.2.1]heptanyl (C7), bicyclo[2.2.2]octanyl (C8), and the like. Exemplary C₃₋₁₀ carbocyclyl groups include, without limitation, the aforementioned C₃₋₈ carbocyclyl groups as well as cyclononyl (C9), cyclononenyl (C9), cyclodecyl (C10), cyclodecenyl (C10), octahydro-1H-indenyl (C9), decahydronaphthalenyl (C10), spiro[4.5]decanyl (C10), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or partially unsaturated.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “halocycloalkyl” refers to a cycloalkyl group that is substituted with at least one halogen.

The term “cycloalkylene” refers to a diradical of a cycloalkyl group. Exemplary cycloalkylene groups include

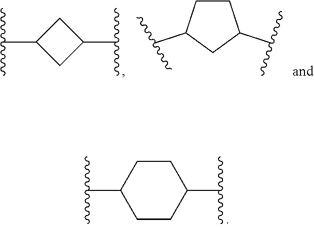

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The term “hydroxyfluoroalkyl” refers to a hydroxyalkyl that is substituted with at least one fluoro.

The term “aralkyl” refers to an alkyl group substituted with an aryl group. Exemplary aralkyl groups include

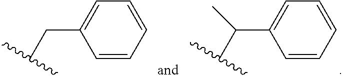

The term “heteroaralkyl” refers to an alkyl group substituted with a heteroaryl group.

The terms “alkenyl” and “alkynyl” are art-recognized and refer to unsaturated aliphatic groups analogous in length and possible substitution to the alkyls described above, but that contain at least one double or triple bond respectively.

The term “cycloalkenyl” refers to a monovalent unsaturated cyclic, bicyclic, or bridged (e.g., adamantyl) carbocyclic hydrocarbon containing at least one C—C double bond.

In certain embodiments, the cycloalkenyl contains 5-10, 5-8, or 5-6 carbons, referred to herein, e.g., as “C₅-C₆ cycloalkenyl”. Exemplary cycloalkenyl groups include cyclohexenyl and cyclopentenyl.

The term “aryl” is art-recognized and refers to a carbocyclic aromatic group. Representative aryl groups include phenyl, naphthyl, anthracenyl, and the like. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “aryl” also includes polycyclic aromatic ring systems having two or more carbocyclic rings in which two or more carbons are common to two adjoining rings (the rings are “fused rings”) wherein all of the fused rings are aromatic rings, e.g., in a naphthyl group.

The term “phenylene” refers to a diradical of a phenyl group. Exemplary phenylene groups include

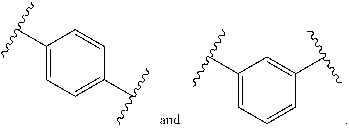

The term “heteroaryl” refers to a radical of a 5-10 membered monocyclic or bicyclic 4n+2 aromatic ring system (e.g., having 6 or 10 n electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

The term “heteroarylene” refers to a diradical of a heteroaryl group. Exemplary heteroarylene groups include: phenylene, pyridinylene, pyridazinylene, pyrimidinylene, pyrazinylene,

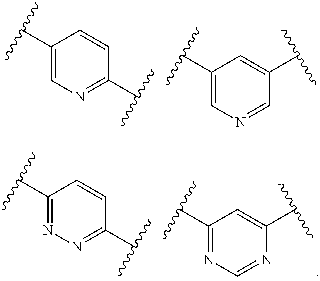

The terms ortho, meta, and para are art-recognized and refer to 1,2-, 1,3- and 1,4-disubstituted benzenes, respectively. For example, the names 1,2-dimethylbenzene and ortho-dimethylbenzene are synonymous.

The term “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits.

A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl.

Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl.

Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C6 aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The term “heterocycloalkyl” refers to a saturated heterocyclyl group having, for example, 3-7 ring atoms selected from carbon and heteroatoms (e.g., O, N, or S).

The terms “amine” and “amino” are art-recognized and refer to both unsubstituted and substituted amines, e.g., a moiety that may be represented by the general formulas:

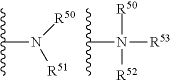

wherein R⁵⁰, R⁵¹, R⁵² and R⁵³ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R^{6′} represents an aryl, a cycloalkyl, a cycloalkenyl, a heterocycle or a polycycle; and m is zero or an integer in the range of 1 to 8. In certain embodiments, only one of R⁵⁰ or R⁵¹ may be a carbonyl, e.g., R⁵⁰, R⁵¹ and the nitrogen together do not form an imide. In other embodiments, R⁵⁰ and R⁵¹ (and optionally R⁵²) each independently represent a hydrogen, an alkyl, an alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and —O—(CH₂)ₘ—R⁶¹, where m and R⁶¹ are described above.

The term “fluoroalkoxyl” refers to an alkoxyl group that is substituted with at least one fluoro group. Exemplary fluoroalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane substituted with an oxo group is cyclopentanone.

The symbols “ [symbol] ”, “*”, and “**” indicate a point of attachment.

The term “substituted” means that one or more hydrogens on the atoms of the designated group are replaced with a selection from the indicated group, provided that the atoms' normal valences under the existing circumstances are not exceeded, and that the substitution results in a stable compound. Combinations of substituents and/or variables are permissible only if such combinations result in stable compounds. The terms “stable compound” or “stable structure” refer to a compound that is sufficiently robust to survive isolation to a useful degree of purity from a reaction mixture, and formulation into an efficacious therapeutic agent.

When any substituent or variable occurs more than one time in any constituent or the compound of the invention, its definition on each occurrence is independent of its definition at every other occurrence, unless otherwise indicated.

It should also be noted that any carbon as well as heteroatom with unsatisfied valences in the text, schemes, examples and tables herein is assumed to have the sufficient number of hydrogen atom(s) to satisfy the valences.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules.

This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

Certain compounds contained in compositions of the present invention may exist in particular geometric or stereoisomeric forms. Further, certain compounds described herein may be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. The compounds may contain one or more stereogenic centers. For example, asymmetric carbon atoms may be present in a substituent such as an alkyl group. All such isomers, as well as mixtures thereof, are intended to be included in this invention, such as, for example, racemic mixtures, single enantiomers, diastereomeric mixtures and individual diastereomers. Additional asymmetric centers may be present depending upon the nature of the various substituents on the molecule. Each such asymmetric center will independently produce two optical isomers, and it is intended that all of the possible optical isomers, diastereomers in mixtures, and pure or partially purified compounds are included within the ambit of this invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate (also known as toluenesulfonate), undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. Further examples of salts include, but are not limited to, ascorbate, borate, nitrate, phosphate, salicylate, and sulfate. Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66 (1) 1-19; P. Gould, International J. of Pharmaceutics (1986) 33.201-217; Anderson et al., The Practice of Medicinal Chemistry (1996), Academic Press, New York; and in The Orange Book (Food & Drug Administration, Washington, D.C. on their website). These disclosures are incorporated herein by reference.

Additional exemplary basic salts include, but are not limited to, ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

The present invention includes the compounds of the invention in all their isolated forms (such as any solvates, hydrates, stereoisomers, and tautomers thereof). Further, the invention includes compounds in which one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques known to those skilled in the art or by processes analogous to those described in the Schemes and Examples herein using appropriate isotopically-enriched reagents and/or intermediates.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

The terms “a” and “an” as used herein mean “one or more” and include the plural unless the context is inappropriate.

As a general matter, compositions specifying a percentage are by weight unless otherwise specified.

### Compounds

In one aspect, provided herein are compounds represented by Formula (I):

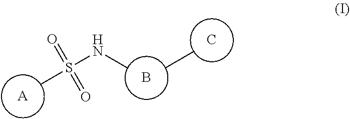

or a pharmaceutically acceptable salt thereof, wherein:
A is selected from the group consisting of phenyl, 5-6 membered heteroaryl, 7-10 membered carbocyclyl, and 5-6 membered heterocyclyl, wherein the phenyl, 5-6 membered heteroaryl, 7-10 membered carbocyclyl, or 5-6 membered heterocyclyl is substituted on one or more available carbons by one, two, three, or more substituents each independently selected from R¹;
B is phenylene or 5-6 membered heteroarylene, wherein the phenylene or the 5-6 membered heteroarylene may be optionally substituted on one or more available carbons by one, two, three, or more substituents each independently selected from R²;
C is selected from the group consisting of 7-10 membered bicyclic heterocyclyl, 5-8 membered heteroaryl, 8 membered bicyclic heteroaryl, and 9 membered bicyclic heteroaryl selected from the group consisting of

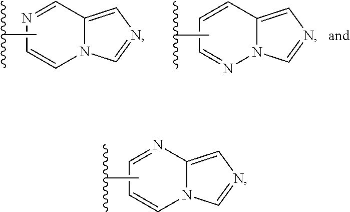

wherein the 7-10 membered bicyclic heterocyclyl, 5-8 membered heteroaryl, 8 membered bicyclic heteroaryl, or 9 membered bicyclic heteroaryl may be optionally substituted on one or more available carbons by one, two, three, or more substituents each independently selected from R³; and wherein, if the 7-10 membered heterocyclyl, 5-8 membered heteroaryl, or 9 membered bicyclic heteroaryl contains a substitutable ring nitrogen atom, that ring nitrogen atom may be optionally substituted by C₁₋₆alkyl;
R¹ is independently, for each occurrence, selected from the group consisting of hydrogen, halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from Ria;
R² is independently, for each occurrence, selected from the group consisting of halogen, methyl, cyano C₁₋₆alkyl, cyano, C₁₋₆alkoxyl, and C₃₋₆cycloalkyl, wherein the C₁₋₆alkyl and C₃₋₆ cycloalkyl may be optionally substituted on one or more available carbons by one, two, three, or more substituents each independently selected from R^{2a};
R³ is independently, for each occurrence, selected from the group consisting of halogen, C₁₋₆alkyl, C₃₋₆cycloalkyl, cyano, C₁₋₆alkoxyl, hydroxyl, oxo, phenyl, —C(O)N(R^{A})(R^{B}), —N(R^{A})(R^{B}), 5-10 membered heterocyclyl, and 5-10 membered heteroaryl, wherein the C₁₋₆ alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl;
R^{A} is independently, for each occurrence, hydrogen or C₁₋₆alkyl;
R^{B} is independently, for each occurrence, selected from the group consisting of hydrogen, C₁₋₆alkyl, C₃₋₆cycloalkyl, phenyl, 5-6 membered heterocyclyl, and 5-6 membered heteroaryl, wherein the C₁₋₆alkyl or phenyl may be optionally substituted on one or more available carbons by one, two, three, or more substituents each independently selected from R^{e}; and wherein, if the 5-6 membered heteroaryl or 5-6 membered heterocyclyl contains a substitutable ring nitrogen atom, that ring nitrogen atom may be optionally substituted by C₁₋₆alkyl;
R^{1a} is independently, for each occurrence, selected from the group consisting of halogen, hydroxyl, and phenyl;
R^{2a} is independently, for each occurrence, halogen;
R^{3a} is independently, for each occurrence, selected from the group consisting of halogen, cyano, hydroxyl, C₁₋₆alkyl, phenyl, C₃₋₆cycloalkyl, 5-6 membered heterocyclyl, and —CO₂(C₁₋₆ alkyl), wherein the C₁₋₆alkyl may be optionally substituted on one or more available carbons by one, two, three, or more substituents each independently selected from halogen, hydroxyl, and phenyl; and wherein, if the 5-6 membered heterocyclyl contains a substitutable ring nitrogen atom, that ring nitrogen atom may be optionally substituted by C₁₋₆alkyl;
R^{e} is independently, for each occurrence, selected from the group consisting of halogen, hydroxyl, C₁₋₆alkyl, 3-6 membered cycloalkyl, 5-6 membered heterocyclyl, —N(R^{C})(R^{D}) and —S(O)₂C₁₋₆alkyl, wherein C₁₋₆alkyl or 5-6 membered heterocyclyl may be optionally substituted on one or more available carbons by one, two, three, or more substituents each independently selected from oxo and —N(R^{C})(R^{D}); and
R^{C} and R^{D} are each independently selected from hydrogen and C₁₋₆alkyl.

In another aspect, provided herein are compounds represented by Formula (Ia):

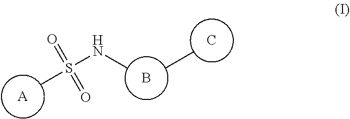

or a pharmaceutically acceptable salt thereof, wherein:
A is selected from the group consisting of phenyl, 5-6 membered heteroaryl, 7-10 membered carbocyclyl, and 5-6 membered heterocyclyl, wherein the phenyl, 5-6 membered heteroaryl, 7-10 membered carbocyclyl, or 5-6 membered heterocyclyl is substituted on one or more available carbons by one, two, three, or more substituents each independently selected from R¹;
B is phenylene or 5-6 membered heteroarylene, wherein the phenylene or 5-6 membered heteroarylene may be optionally substituted on one or more available carbons by one, two, three, or more substituents each independently selected from R²;
C is selected from the group consisting of 7-10 membered heterocyclyl, 5-8 membered heteroaryl, 9 membered bicyclic heterocyclyl, or 8-9 membered bicyclic heteroaryl selected from the group consisting of N

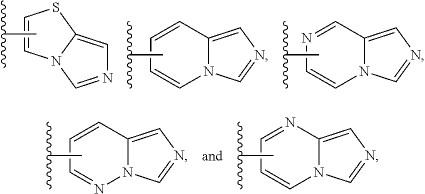

wherein the 7-10 membered heterocyclyl, 5-8 membered heteroaryl, 9 membered bicyclic heterocyclyl, or 8-9 membered bicyclic heteroaryl may be optionally substituted on one or more available carbons by one, two, three, or more substituents each independently selected from R³; and wherein, if the 7-10 membered heterocyclyl contains a substitutable ring nitrogen atom, that ring nitrogen atom may be optionally substituted by C₁₋₆alkyl;
R¹ is independently, for each occurrence, selected from the group consisting of halogen, C₁₋₆alkyl, C₃₋₆cycloalkyl, cyano, hydroxyl, oxo, C₁₋₆alkoxyl, and —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl or C₁₋₆alkoxyl may be optionally substituted on one or more available carbons by one, two, three, or more substituents each independently selected from R^{1a};
R² is independently, for each occurrence, selected from the group consisting of halogen, C₁₋₆alkyl, and cyano;
R³ is independently, for each occurrence, selected from the group consisting of halogen, C₁₋₆alkyl, C₁₋₆alkoxyl, oxo, —C(O)N(R^{A})(R^{B}), —N(R^{A})(R^{B}), 5-10 membered heterocyclyl, and 5-10 membered heteroaryl, wherein the C₁₋₆alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl;
R^{A} is independently, for each occurrence, hydrogen or C₁₋₆alkyl;
R^{B} is independently, for each occurrence, selected from the group consisting of hydrogen, C₁₋₆alkyl, phenyl, 5-6 membered heterocyclyl, and 5-6 membered heteroaryl, wherein the C₁₋₆alkyl or phenyl may be optionally substituted on one or more available carbons by one, two, three, or more substituents each independently selected from R^{e}; and wherein, if the 5-6 membered heteroaryl or 5-6 membered heterocyclyl contains a substitutable ring nitrogen atom, that ring nitrogen atom may be optionally substituted by C₁₋₆alkyl;
R^{1a} is independently, for each occurrence, selected from the group consisting of halogen, hydroxyl, and phenyl;
R^{3a} is independently, for each occurrence, selected from the group consisting of halogen, cyano, hydroxyl, C₁₋₆alkyl, phenyl, C₃₋₆cycloalkyl, 5-6 membered heterocyclyl, and —CO₂(C₁₋₆alkyl), wherein the C₁₋₆alkyl may be optionally substituted on one or more available carbons by one, two, three, or more substituents each independently selected from halogen, hydroxyl, and phenyl; and wherein, if the 5-6 membered heterocyclyl contains a substitutable ring nitrogen atom, that ring nitrogen atom may be optionally substituted by C₁₋₆alkyl;
R^{e} is independently, for each occurrence, selected from the group consisting of halogen, hydroxyl, C₁₋₆alkyl, 3-6 membered cycloalkyl, 5-6 membered heterocyclyl, —N(R^{C})(R^{D}), and —S(O)₂C₁₋₆alkyl, wherein C₁₋₆alkyl or 5-6 membered heterocyclyl may be optionally substituted on one or more available carbons by one, two, three, or more substituents each independently selected from oxo and —N(R^{C})(R^{D}); and
R^{C} and R^{D} are each independently C₁₋₆alkyl.

In some embodiments, A is selected from the group consisting of phenyl, pyridyl,

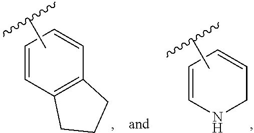

wherein A is substituted with one or two independent R¹ substituents selected from the group consisting of halogen, C₁₋₆alkyl, C₃₋₆cycloalkyl, cyano, hydroxyl, oxo, C₁₋₆alkoxyl, and —O—C₃₋₆cycloalkyl, wherein the C₁₋₆alkyl or C₁₋₆alkoxyl may be optionally substituted on one or more available carbons by one, two, three, or more substituents each independently selected from halogen or phenyl.

In some embodiments, A is selected from the group consisting of phenyl,

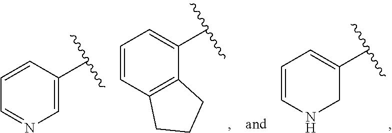

wherein A is substituted with two independent R¹ substituents selected from the group consisting of halogen, C₁₋₆ alkyl, cyano, hydroxyl, oxo, C₁₋₆alkoxyl, and —O—C₃₋₆cycloalkyl, wherein the C₁₋₆alkyl or C₁₋₆ alkoxyl may be optionally substituted on one or more available carbons by one, two, three, or more substituents each independently selected from halogen or phenyl.

In some embodiments, R¹ is independently, for each occurrence, selected from the group consisting of chloro, fluoro, cyano, hydroxyl, oxo, CH₃, CF₃, —O—CH₃, —O—CH₂CH₃, —O—CH(CH₃)₂, —O—CH₂CH(CH₃)₂, —O—CH₂CF₃,

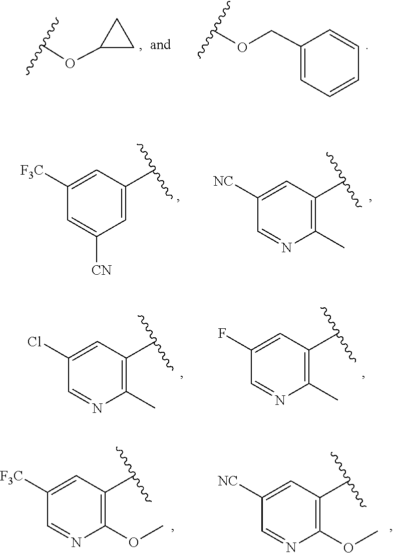

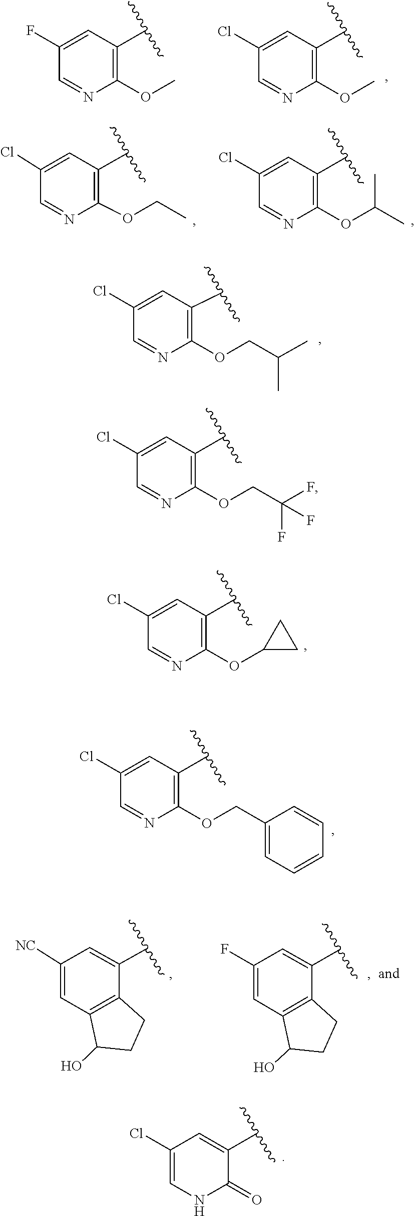

In some embodiments, B is phenylene or pyridylene, wherein B may be optionally substituted with one or two independent R² substituents selected from the group consisting of halogen, C₁₋₆alkyl, and cyano.

In some embodiments, B is

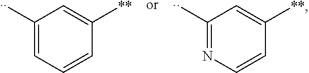

wherein * denotes the point of attachment to

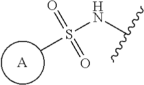

and * * denotes the point of attachment to

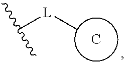

wherein B may be optionally substituted with one or two independent R² substituents selected from the group consisting of halogen, C₁₋₆alkyl, and cyano.

In some embodiments, B is

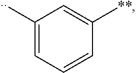

wherein B is substituted with one R² substituent selected from the group consisting of halogen, C₁₋₆alkyl, and cyano.

In some embodiments, R² is fluoro.

In some embodiments, B is

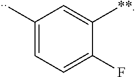

In some embodiments, B is

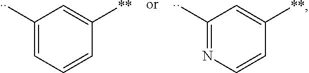

wherein B is substituted with two independent R² substituents selected from the group consisting of halogen, C₁₋₆alkyl, and cyano.

In some embodiments, R² is independently, for each occurrence, selected from the group consisting of chloro, fluoro, cyano, and CH₃.

In some embodiments, B is selected from the group consisting of

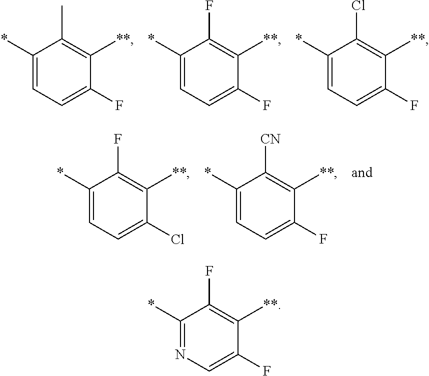

In another aspect, provided herein are compounds represented by Formula (Ia):

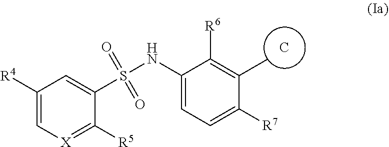

or a pharmaceutically acceptable salt thereof, wherein:
C is selected from 7-10 membered bicyclic heterocyclyl, 8 membered bicyclic heteroaryl, or 9-membered bicyclic heteroaryl selected from the group consisting of

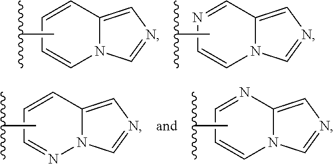

wherein the 7-10 membered bicyclic heterocyclyl, 8 membered bicyclic heteroaryl, or 9 membered bicyclic heteroaryl may be optionally substituted on one or more available carbons by one, two, three, or more substituents each independently selected from R³; wherein the 7-10 membered bicyclic heterocyclyl is partially unsaturated and contains at least two nitrogen atoms; wherein the 8 membered bicyclic heteroaryl contains at least two nitrogen items; wherein, if the 7-10 membered bicyclic heterocyclyl, 8 membered bicyclic heteroaryl, or 9 membered bicyclic heteroaryl contains a substitutable ring nitrogen atom, that ring nitrogen atom may be optionally substituted by C₁₋₆alkyl;
X is selected from the group consisting of CH, C(R⁸), and N;
R³ is independently, for each occurrence, selected from the group consisting of halogen, C₁₋₆alkyl, C₃₋₆cycloalkyl, cyano, C₁₋₆alkoxyl, hydroxyl, oxo, phenyl, —C(O)N(R^{A})(R^{B}), —N(R^{A})(R^{B}), 5-10 membered heterocyclyl, and 5-10 membered heteroaryl, wherein the C₁₋₆ alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl;
R⁴ is selected from the group consisting of halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{4a};
R⁵ is selected from the group consisting of hydrogen, halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{5a};
R⁶ is selected from the group consisting of halogen, C₁₋₆alkyl, cyano, C₁₋₆alkoxyl, and C₃₋₆cycloalkyl, wherein the C₁₋₆alkyl and C₁₋₆cycloalkyl may be optionally substituted on one or more available carbons by one, two, three, or more substituents each independently selected from R^{6a};
R⁷ is selected from the group consisting of fluoro, chloro, methyl, and cyano;
R⁸ is selected from is selected from the group consisting of halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{8a};
or when X is C(R⁸), R⁸ and R⁵ may optionally combine together with the atoms to which they are attached to form a 3-7 membered carbocyclyl, 3-7 membered heterocyclyl, or 5-10 membered heteroaryl, wherein the 3-7 membered carbocyclyl, 3-7 membered heterocyclyl, or 5-10 membered heteroaryl may be optionally substituted on one or more available carbons by one, two, three, or more hydroxyl substituents; wherein, if the 3-7 membered carbocyclyl, 3-7 membered heterocyclyl, or 5-10 membered heteroaryl contains a substitutable ring nitrogen atom, that ring nitrogen atom may be optionally substituted by C₁₋₆alkyl;
R^{A} is hydrogen or C₁₋₆alkyl;
R^{B} is selected from the group consisting of hydrogen, C₁₋₆alkyl, C₃₋₆cycloalkyl, phenyl, 5-6 membered heterocyclyl, and 5-6 membered heteroaryl, wherein the C₁₋₆alkyl or phenyl may be optionally substituted on one or more available carbons by one, two, three, or more substituents each independently selected from R^{e}; and wherein, if the 5-6 membered heteroaryl or 5-6 membered heterocyclyl contains a substitutable ring nitrogen atom, that ring nitrogen atom may be optionally substituted by C₁₋₆alkyl;
R^{3a} is independently, for each occurrence, selected from the group consisting of halogen, cyano, hydroxyl, C₁₋₆alkyl, phenyl, C₃₋₆cycloalkyl, 5-6 membered heterocyclyl, and —CO₂(C₁₋₆ alkyl), wherein the C₁₋₆alkyl may be optionally substituted on one or more available carbons by one, two, three, or more substituents each independently selected from halogen, hydroxyl, and phenyl; and wherein, if the 5-6 membered heterocyclyl contains a substitutable ring nitrogen atom, that ring nitrogen atom may be optionally substituted by C₁₋₆alkyl;
R^{4a} is independently, for each occurrence, selected from the group consisting of halogen, hydroxyl, and phenyl;
R⁵, is, for each occurrence, selected from the group consisting of halogen, hydroxyl, and phenyl;
R^{6a} is independently, for each occurrence, halogen;
R^{8a} is independently, for each occurrence, selected from the group consisting of halogen, hydroxyl, and phenyl;
R^{e} is independently, for each occurrence, selected from the group consisting of halogen, hydroxyl, C₁₋₆alkyl, 3-6 membered cycloalkyl, 5-6 membered heterocyclyl, —N(R^{C})(R^{D}), and —S(O)₂C₁₋₆alkyl, wherein C₁₋₆alkyl or 5-6 membered heterocyclyl may be optionally substituted on one or more available carbons by one, two, three, or more substituents each independently selected from oxo and —N(R^{C})(R^{D}); and
R^{C} and R^{D} are each independently selected from hydrogen and C₁₋₆alkyl; wherein the compound of Formula (Ib) is not

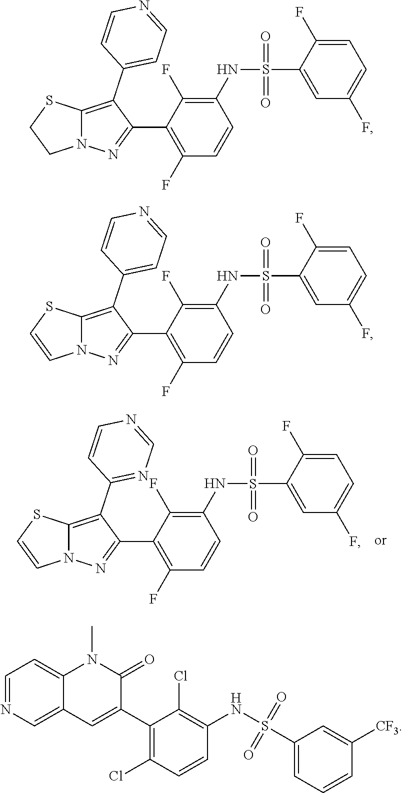

In another aspect, provided herein are compounds represented by Formula (Ia):

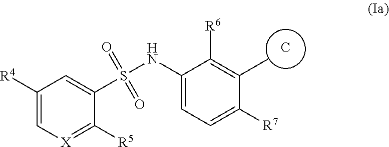

or a pharmaceutically acceptable salt thereof, wherein:
C is selected from 9-membered bicyclic heterocyclyl and 8-9 membered bicyclic heteroaryl selected from the group consisting of

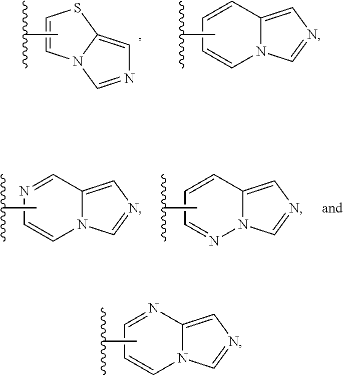

wherein the 9 membered bicyclic heterocyclyl or 8-9 membered bicyclic heteroaryl may be optionally substituted on one or more available carbons by one, two, three, or more substituents each independently selected from R³; wherein the 9 membered bicyclic heterocyclyl is partially unsaturated and contains at least two nitrogen atoms; wherein, if the 9 membered bicyclic heterocyclyl or 8-9 membered bicyclic heteroaryl contains a substitutable ring nitrogen atom, that ring nitrogen atom may be optionally substituted by C₁₋₆alkyl;
X is selected from the group consisting of CH, C(R⁸) and N;
R³ is independently, for each occurrence, selected from the group consisting of halogen, C₁₋₆alkyl, C₃₋₆cycloalkyl, cyano, C₁₋₆alkoxyl, hydroxyl, oxo, phenyl, —C(O)N(R^{A})(R^{B}), —N(R^{A})(R^{B}), 5-10 membered heterocyclyl, and 5-10 membered heteroaryl, wherein the C₁₋₆ alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl;
R⁴ is selected from the group consisting of halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{4a};
R⁵ is selected from the group consisting of halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{5a};
R⁶ is selected from the group consisting of halogen, C₁₋₆alkyl, cyano, C₁₋₆alkoxyl, and C₃₋₆cycloalkyl, wherein the C₁₋₆alkyl and C₁₋₆cycloalkyl may be optionally substituted on one or more available carbons by one, two, three, or more substituents each independently selected from R^{6a};
R⁷ is selected from the group consisting of fluoro, chloro, methyl, and cyano;
R⁸ is selected from is selected from the group consisting of halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{8a};
or when X is C(R⁸), R⁸ and R⁵ may optionally combine together with the atoms to which they are attached to form a 3-7 membered carbocyclyl, 3-7 membered heterocyclyl, or 5-10 membered heteroaryl, wherein the 3-7 membered carbocyclyl, 3-7 membered heterocyclyl, or 5-10 membered heteroaryl may be optionally substituted on one or more available carbons by one, two, three, or more hydroxyl substituents; wherein, if the 3-7 membered carbocyclyl, 3-7 membered heterocyclyl, or 5-10 membered heteroaryl contains a substitutable ring nitrogen atom, that ring nitrogen atom may be optionally substituted by C₁₋₆alkyl;
R^{A} is hydrogen or C₁₋₆alkyl;
R^{B} is selected from the group consisting of hydrogen, C₁₋₆alkyl, C₃₋₆cycloalkyl, phenyl, 5-6 membered heterocyclyl, and 5-6 membered heteroaryl, wherein the C₁₋₆alkyl or phenyl may be optionally substituted on one or more available carbons by one, two, three, or more substituents each independently selected from R^{e}; and wherein, if the 5-6 membered heteroaryl or 5-6 membered heterocyclyl contains a substitutable ring nitrogen atom, that ring nitrogen atom may be optionally substituted by C₁₋₆alkyl;
R^{3a} is independently, for each occurrence, selected from the group consisting of halogen, cyano, hydroxyl, C₁₋₆alkyl, phenyl, C₃₋₆cycloalkyl, 5-6 membered heterocyclyl, and —CO₂(C₁₋₆ alkyl), wherein the C₁₋₆alkyl may be optionally substituted on one or more available carbons by one, two, three, or more substituents each independently selected from halogen, hydroxyl, and phenyl; and wherein, if the 5-6 membered heterocyclyl contains a substitutable ring nitrogen atom, that ring nitrogen atom may be optionally substituted by C₁₋₆alkyl;
R^{4a} is independently, for each occurrence, selected from the group consisting of halogen, hydroxyl, and phenyl;
R^{5a} is independently, for each occurrence, selected from the group consisting of halogen, hydroxyl, and phenyl;
R^{6a} is independently, for each occurrence, halogen;
R^{8a} is independently, for each occurrence, selected from the group consisting of halogen, hydroxyl, and phenyl;
R^{e} is independently, for each occurrence, selected from the group consisting of halogen, hydroxyl, C₁₋₆alkyl, 3-6 membered cycloalkyl, 5-6 membered heterocyclyl, —N(R^{C})(R^{D}), and —S(O)₂C₁₋₆alkyl, wherein C₁₋₆alkyl or 5-6 membered heterocyclyl may be optionally substituted on one or more available carbons by one, two, three, or more substituents each independently selected from oxo and —N(R^{C})(R^{D}); and
R^{C} and R^{D} are each independently selected from hydrogen and C₁₋₆alkyl.

In another aspect, provided herein are compounds represented by Formula (Ia):

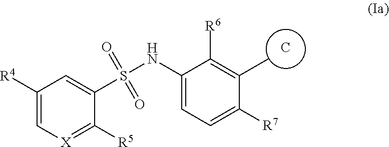

or a pharmaceutically acceptable salt thereof, wherein:
C is selected from 9-membered bicyclic heterocyclyl and 8-9 membered bicyclic heteroaryl selected from the group consisting of

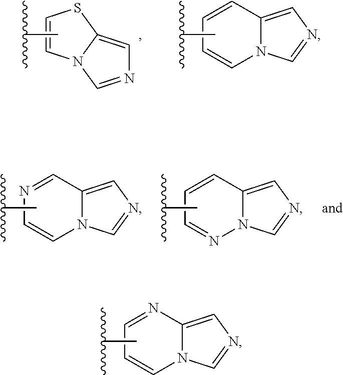

wherein the 9-membered bicyclic heterocyclyl or 8-9 membered bicyclic heteroaryl may be optionally substituted on one or more available carbons by one, two, three, or more substituents each independently selected from R³; wherein the 9 membered bicyclic heterocyclyl is partially unsaturated and contains at least two nitrogen atoms; wherein, if the 9 membered bicyclic heterocyclyl or 8-9 membered bicyclic heteroaryl contains a substitutable ring nitrogen atom, that ring nitrogen atom may be optionally substituted by C₁₋₆alkyl;
X is selected from the group consisting of CH, C(R⁸) and N;
R³ is independently, for each occurrence, selected from the group consisting of halogen, C₁₋₆alkyl, C₁₋₆alkoxyl, oxo, —C(O)N(R^{A})(R^{B}), —N(R^{A})(R^{B}), 5-10 membered heterocyclyl, and 5-10 membered heteroaryl, wherein the C₁₋₆alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl;
R⁴ is selected from the group consisting of halogen, C₁₋₆alkyl, and cyano, wherein the C₁₋₆alkyl may be optionally substituted on one or more available carbons by one, two, three, or more substituents each independently selected from R^{4a};
R⁵ is selected from the group consisting of C₁₋₆alkyl, cyano, hydroxyl, C₁₋₆alkoxyl, and —O—C₃₋₆cycloalkyl, wherein the C₁₋₆alkyl and C₁₋₆alkoxyl may be optionally substituted on one or more available carbons by one, two, three, or more substituents each independently selected from R^{5a};
R⁸ is cyano;
or when X is C(R⁸), R⁸ and R⁵ may optionally combine together with the atoms to which they are attached to form a 3-7 membered carbocyclyl, wherein the 3-7 membered carbocyclyl may be optionally substituted on one or more available carbons by one, two, three, or more hydroxyl substituents;
R⁶ is selected from the group consisting of hydrogen, halogen, C₁₋₆alkyl, and cyano, wherein the C₁₋₆alkyl may be optionally substituted on one or more available carbons by one, two, three, or more substituents each independently selected from R^{6a};
R⁷ is halogen;
R^{A} is hydrogen;
R^{B} is selected from the group consisting of hydrogen, C₁₋₆alkyl, phenyl, 5-6 membered heterocyclyl, and 5-6 membered heteroaryl, wherein the C₁₋₆alkyl or phenyl may be optionally substituted on one or more available carbons by one, two, three, or more substituents each independently selected from R^{e}; and wherein, if the 5-6 membered heteroaryl or 5-6 membered heterocyclyl contains a substitutable ring nitrogen atom, that ring nitrogen atom may be optionally substituted by C₁₋₆alkyl;
R^{3a} is independently, for each occurrence, selected from the group consisting of halogen, cyano, hydroxyl, C₁₋₆alkyl, phenyl, C₃₋₆cycloalkyl, 5-6 membered heterocyclyl, and —CO₂(C₁₋₆alkyl), wherein the C₁₋₆alkyl may be optionally substituted on one or more available carbons by one, two, three, or more substituents each independently selected from halogen, hydroxyl, and phenyl; and wherein, if the 5-6 membered heterocyclyl contains a substitutable ring nitrogen atom, that ring nitrogen atom may be optionally substituted by C₁₋₆alkyl;
R^{4a} is independently, for each occurrence, halogen;
R^{5a} is independently, for each occurrence, selected from halogen and phenyl;
R^{6a} is halogen;
R^{e} is independently, for each occurrence, selected from the group consisting of halogen, hydroxyl, C₁₋₆alkyl, 3-6 membered cycloalkyl, 5-6 membered heterocyclyl, —N(R^{c})(R), and —S(O)₂C₁₋₆alkyl, wherein the C₁₋₆alkyl or 5-6 membered heterocyclyl may be optionally substituted on one or more available carbons by one, two, three, or more substituents each independently selected from oxo and —N(R^{C})(R^{D}); and
R^{C} and R^{D} are each independently selected from hydrogen and C₁₋₆alkyl.

In some embodiments, R⁴ is independently selected from the group consisting of chloro, fluoro, cyano, hydroxyl, oxo, CH₃, CF₃, —O—CH₃, —O—CH₂CH₃, —O—CH(CH₃)₂, —O—CH₂CH(CH₃)₂, —O—CH₂CF₃,

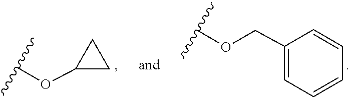 [SMILES: *OC1CC1 ; *OCc1ccccc1]

In some embodiments, R⁴ is selected from the group consisting of chloro, fluoro, cyano, and CF₃.

In some embodiments, R⁵ is selected from the group consisting of chloro, fluoro, cyano, hydroxyl, oxo, CH₃, CF₃, —O—CH₃, —O—CH₂CH₃, —O—CH(CH₃)₂, —O—CH₂CH(CH₃)₂, —O—CH₂CF₃,

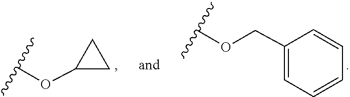 [SMILES: *OC1CC1 ; *OCc1ccccc1]

In some embodiments, R⁵ is selected from the group consisting of CH₃, —O—CH₃, —O—CH₂—CH₃, —O—CH₂—CF₃, —O—CH₂—C(H)(CH₃)₂, —O—CH—(CH₃)₂,

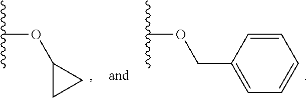 [SMILES: *OC1CC1 ; *OCc1ccccc1]

In some embodiments, X is N.

In some embodiments, X is (R⁸).

In some embodiments, R⁸ and R⁵ are taken together with the atoms to which they are attached to form a 3-7 membered carbocyclyl, wherein the 3-7 membered carbocyclyl may be optionally substituted with hydroxyl. In some embodiments, R⁸ and R⁵ are taken together with the atoms to which they are attached to form a 3-7 membered carbocyclyl, wherein the 3-7 membered carbocyclyl is substituted with hydroxyl. In some embodiments, R⁸ and R⁵ are taken together with the atoms to which they are attached to form a 5 membered carbocyclyl, wherein the 5 membered carbocyclyl may be optionally substituted with hydroxyl. In some embodiments, R⁸ and R⁵ are taken together with the atoms to which they are attached to form a 5 membered carbocyclyl, wherein the 5 membered carbocyclyl is substituted with hydroxyl.

In some embodiments, R⁶ is selected from the group consisting of halogen, C₁₋₆alkyl, and cyano. In some embodiments, R⁶ is selected from the group consisting of hydrogen, chloro, fluoro, cyano, and CH₃. In some embodiments, R⁶ is fluoro.

In some embodiments, R⁷ is fluoro or chloro. In some embodiments, R⁷ is fluoro.

In some embodiments, R⁶ is methyl, and R⁷ is fluoro. In some embodiments, R⁶ is fluoro, and R⁷ is fluoro. In some embodiments, R⁶ is chloro, and R⁷ is fluoro. In some embodiments, R⁶ is fluoro, and R⁷ is chloro. In some embodiments, R⁶ is cyano, and R⁷ is fluoro. In some embodiments, R⁶ is methyl, and R⁷ is fluoro.

In another aspect, provided herein are compounds represented by Formula (Ib):

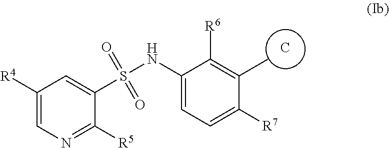

or a pharmaceutically acceptable salt thereof, wherein:
C is 7-10 membered bicyclic heterocyclyl, 5-8 membered bicyclic heteroaryl, or 9 membered bicyclic heteroaryl selected from the group consisting of

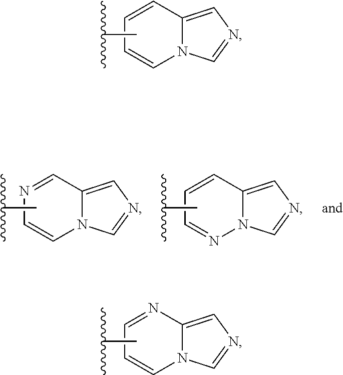

wherein the 7-10 membered bicyclic heterocyclyl, 5-8 membered bicyclic heteroaryl, or 9 membered bicyclic heteroaryl may be optionally substituted on one or more available carbons by one, two, three, or more substituents each independently selected from R³; and wherein, if the 7-10 bicyclic membered heterocyclyl is partially unsaturated and contains at least two nitrogen atoms; wherein the 8 membered bicyclic heteroaryl contains at least two nitrogen items; wherein, if the 7-10 membered bicyclic heterocyclyl, 8 membered bicyclic heteroaryl, or 9 membered bicyclic heteroaryl, that ring nitrogen atom may be optionally substituted by C₁₋₆alkyl;
R³ is independently, for each occurrence, selected from the group consisting of halogen, C₁₋₆alkyl, C₁₋₆alkoxyl, cyano, C₁₋₆alkoxyl, hydroxyl, phenyl, oxo, —C(O)N(R^{A})(R^{B}), —N(R^{A})(R^{B}), 5-10 membered heterocyclyl, and 5-10 membered heteroaryl, wherein the C₁₋₆alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl;
R^{A} is independently, for each occurrence, hydrogen or C₁₋₆alkyl;
R^{B} is independently, for each occurrence, selected from the group consisting of hydrogen, C₁₋₆alkyl, C₃₋₆cycloalkyl, phenyl, 5-6 membered heterocyclyl, and 5-6 membered heteroaryl, wherein the C₁₋₆alkyl or phenyl may be optionally substituted on one or more available carbons by one, two, three, or more substituents each independently selected from R^{e}; and wherein, if the 5-6 membered heteroaryl or 5-6 membered heterocyclyl contains a substitutable ring nitrogen atom, that ring nitrogen atom may be optionally substituted by C₁₋₆alkyl;
R^{3a} is independently, for each occurrence, selected from the group consisting of halogen, cyano, hydroxyl, C₁₋₆alkyl, phenyl, C₃₋₆cycloalkyl, 5-6 membered heterocyclyl, and —CO₂(C₁₋₆alkyl), wherein the C₁₋₆alkyl may be optionally substituted on one or more available carbons by one, two, three, or more substituents each independently selected from group consisting of halogen, hydroxyl, and phenyl; and wherein, if the 5-6 membered heterocyclyl contains a substitutable ring nitrogen atom, that ring nitrogen atom may be optionally substituted by C₁₋₆alkyl;
R^{e} is independently, for each occurrence, selected from the group consisting of halogen, hydroxyl, C₁₋₆alkyl, 3-6 membered cycloalkyl, 5-6 membered heterocyclyl, —N(R^{C})(R^{D}), and —S(O)₂C₁₋₆alkyl, wherein C₁₋₆alkyl or 5-6 membered heterocyclyl may be optionally substituted on one or more available carbons by one, two, three, or more substituents each independently selected from oxo and —N(R^{C})(R^{D});
R^{C} and R^{D} are each independently C₁₋₆alkyl;
R⁴ is selected from the group consisting of halogen, cyano, and C₁₋₆alkyl, wherein the C₁₋₆alkyl may be optionally substituted on one or more available carbons by one, two, three, or more independent halogen substituents;
R⁵ is selected from the group consisting of C₁₋₆alkyl, C₁₋₆alkoxyl, —O—C₃₋₆cycloalkyl, C₃₋₆cycloalkyl, cyano, hydroxyl, oxo, —N(R^{A})(R^{B}), —N(R^{A})—C(O)(R^{B}), —(C₁-6alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆ alkyl) may be optionally substituted on one or more available carbons by one, two, three, or more substituents each independently selected from hydroxyl, halogen and phenyl;
R⁶ is selected from the group consisting of hydrogen, halogen, cyano, C₁₋₆alkyl, C₁₋₆alkoxyl, and C₃₋₆cycloalkyl, wherein the C₁₋₆alkyl and C₃₋₆cycloalkyl may be optionally substituted on one or more available carbons by one, two, three, or more substituents each independently selected from R^{6a};
R^{6a} is halogen; and
R⁷ is selected from the group consisting of fluoro, chloro, methyl, and cyano.

In some embodiments, R⁴ is selected from the group consisting of chloro, fluoro, cyano, and CF₃.

In some embodiments, R⁵ is selected from the group consisting of CH₃, —O—CH₃, —O—CH₂—CH₃, —O—CH₂—CF₃, —O—CH₂—C(H)(CH₃)₂, —O—CH—(CH₃)₂,

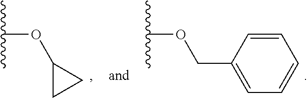 [SMILES: *OC1CC1 ; *OCc1ccccc1]

In some embodiments, R⁶ is selected from the group consisting of hydrogen, chloro, fluoro, cyano, C₁₋₆alkyl, and CH₃.

In some embodiments, R⁷ is fluoro or chloro.

In some embodiments, C is selected from the group consisting of

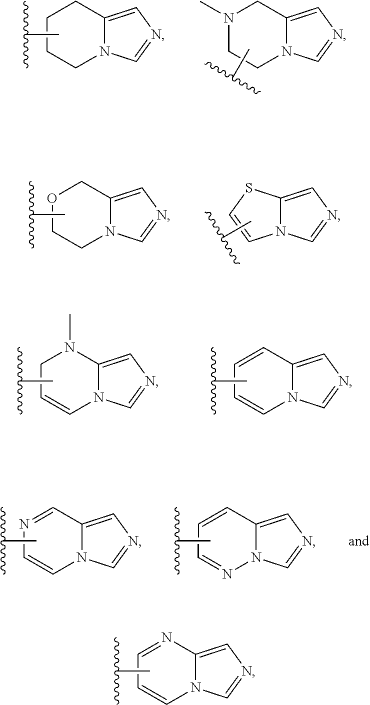

wherein C may be optionally substituted on one or more available carbons by one, two, three, or more independent R³ substituents selected from the group consisting of halogen, C₁₋₆alkyl, C₁₋₆alkoxyl, oxo, —C(O)N(R^{A})(R^{B}), —N(R^{A})(R^{B}), 5-10 membered heterocyclyl, and 5-10 membered heteroaryl, wherein the C₁₋₆alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl.

In some embodiments, C is selected from the group consisting of

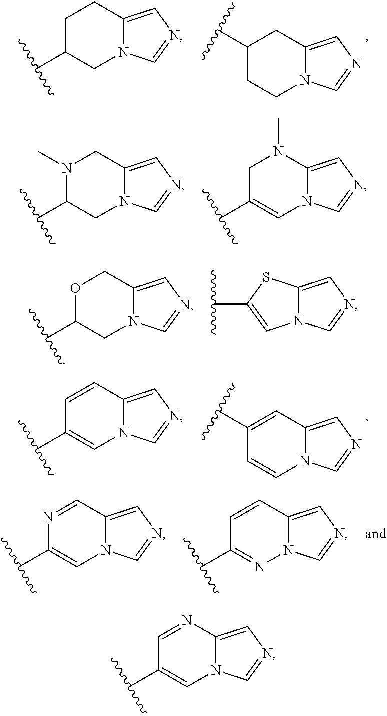

wherein C may be optionally substituted on one or more available carbons by one, two, three, or more independent R³ substituents selected from the group consisting of halogen, C₁₋₆alkyl, C₁₋₆alkoxyl, oxo, —C(O)N(R^{A})(R^{B}), —N(R^{A})(R^{B}), 5-10 membered heterocyclyl, and 5-10 membered heteroaryl, wherein the C₁₋₆alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl.

In some embodiments, C is selected from the group consisting of

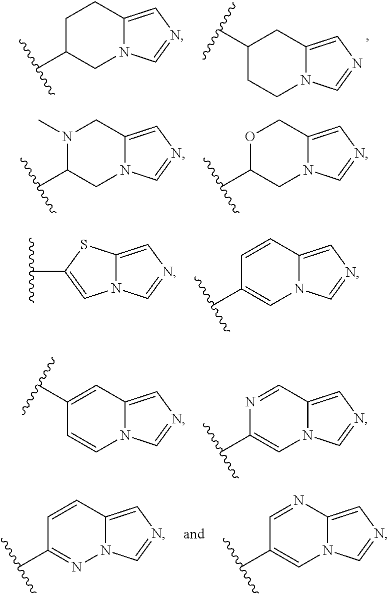

wherein C is substituted with one R³ substituent selected from the group consisting of halogen, C₁₋₆alkyl, C₁₋₆alkoxyl, oxo, —C(O)N(R^{A})(R^{B}), —N(R^{A})(R^{B}), 5-10 membered heterocyclyl, and 5-10 membered heteroaryl, wherein the C₁₋₆alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl.

In some embodiments, R³ is selected from the group consisting of C₁₋₆alkyl, —C(O)N(R^{A})(R^{B}), 5-10 membered heterocyclyl, and 5-10 membered heteroaryl, wherein the C₁₋₆alkyl and 5-10 membered heteroaryl may be optionally substituted on one or more available carbons by one, two, three, or more substituents, for each occurrence, independently selected from the group consisting of chloro, cyano, hydroxyl, CH₃, CF₃, —CH₂CH(CH₃)₂, —CH₂OH, —C(O)OCH₃, cyclopropyl, phenyl,

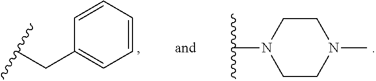

In some embodiments, R^{A} is hydrogen.

In some embodiments, R^{B} is hydrogen, CH₃, —CH₂CH₃, —CH(CH₃)₂, —CH₂CH(CH₃)₂, —CH₂CF₃, CH₂CH₂OH, —CH₂CH₂N(CH₃)₂, —(CH₂)₃N(CH₃)₂, —CH₂CH₂S(O)₂CH₃,

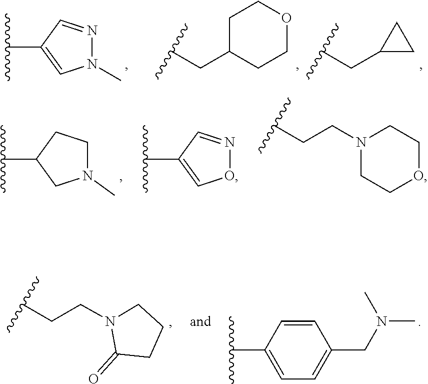

In some embodiments, R³ is selected from the group consisting of —CH₂OH,

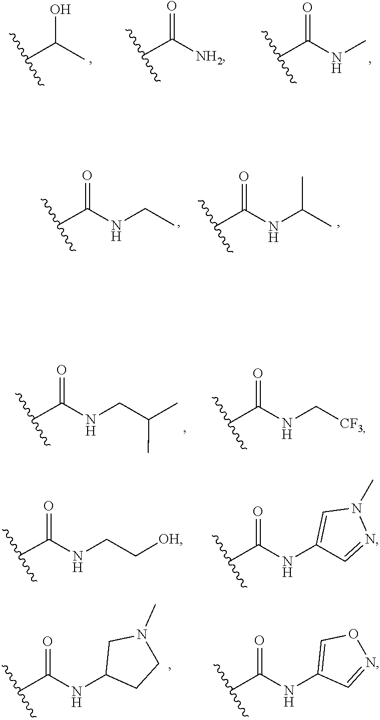

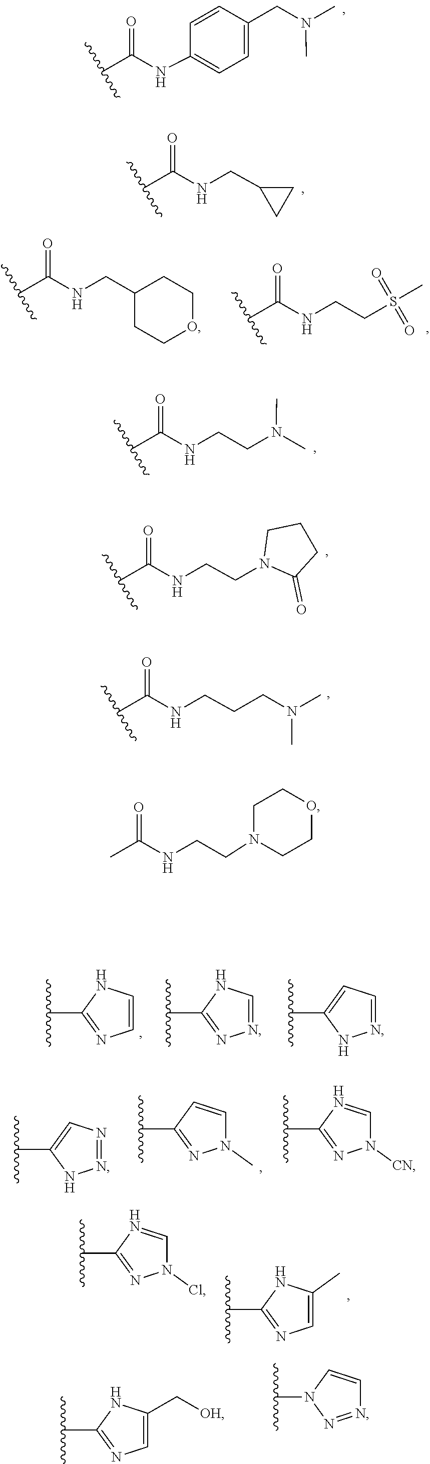

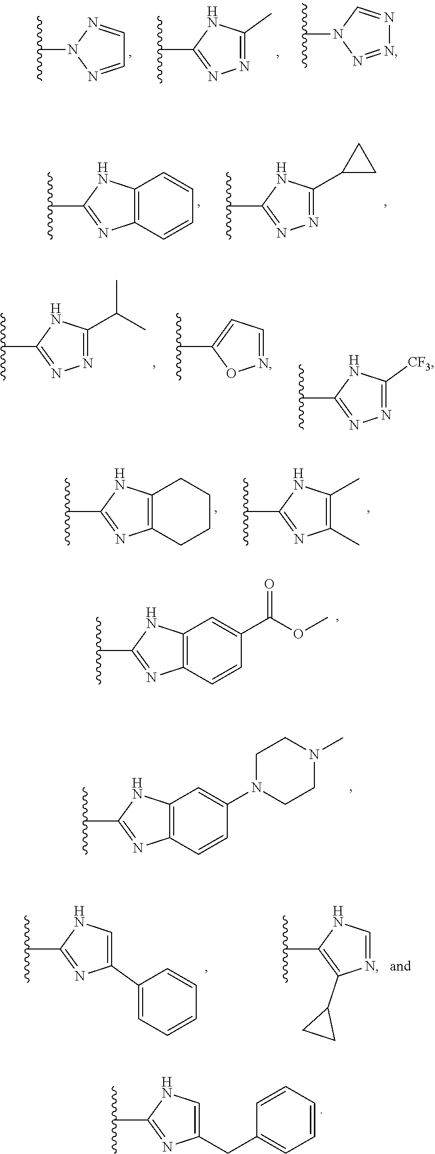

In some embodiments, C is selected from the group consisting of

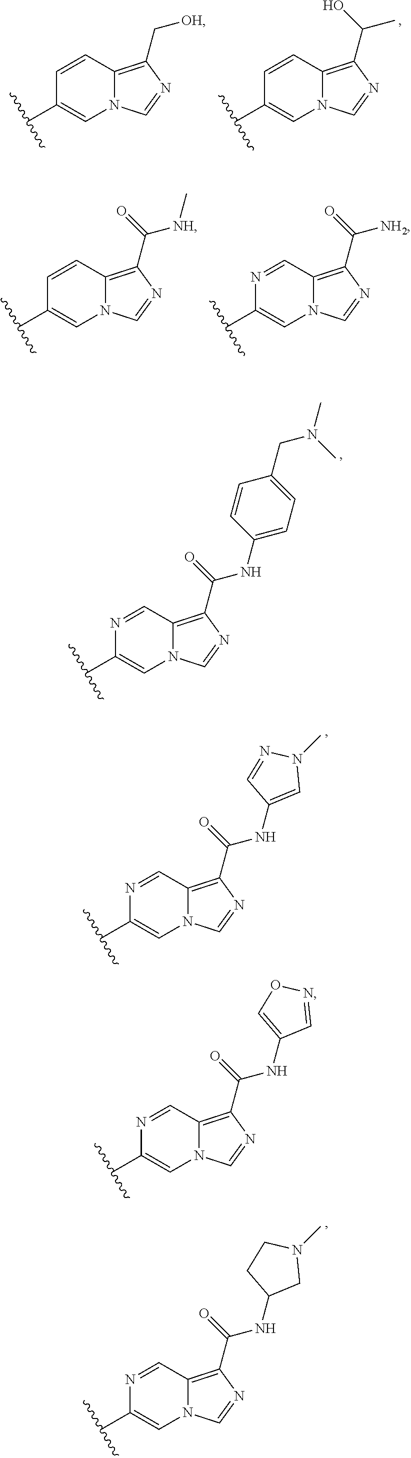

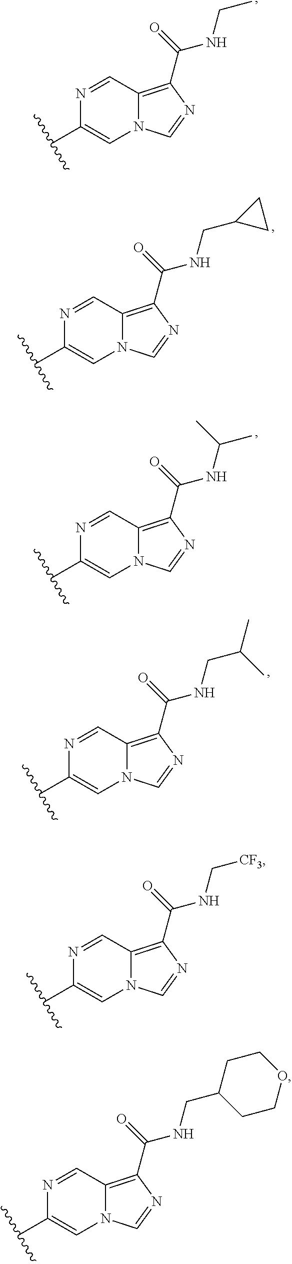

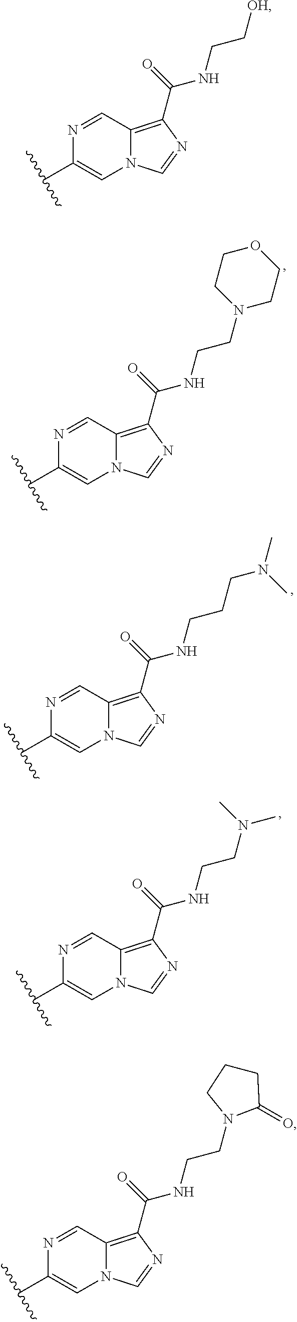

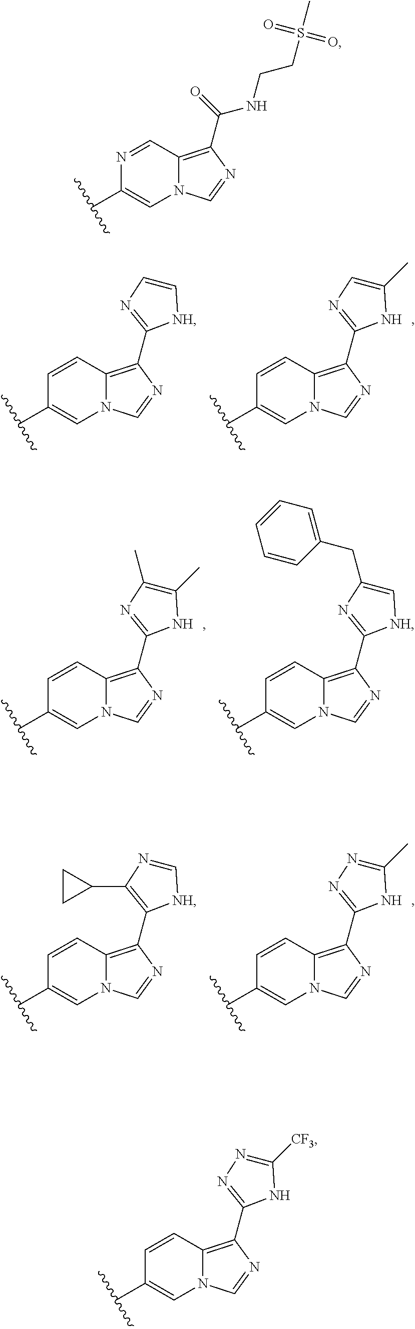

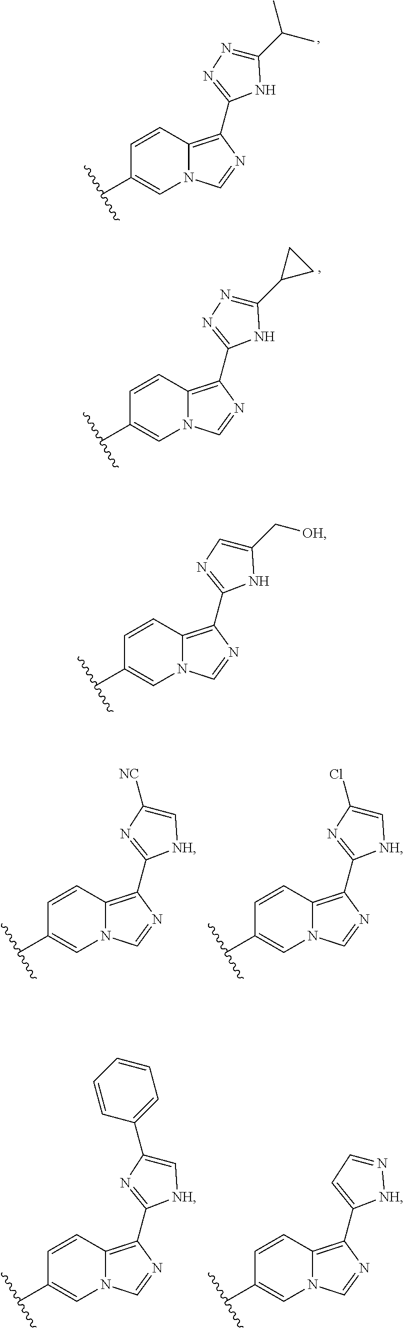

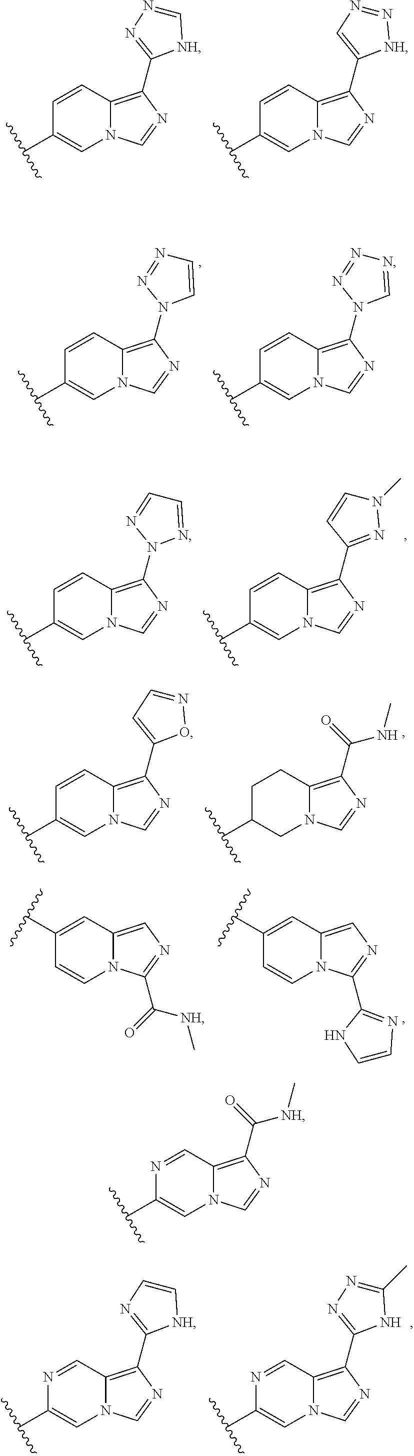

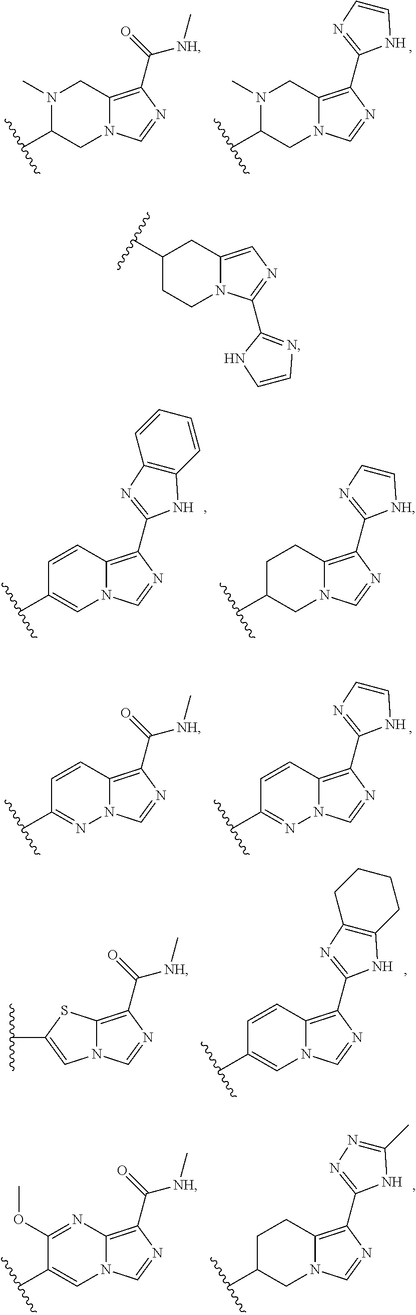

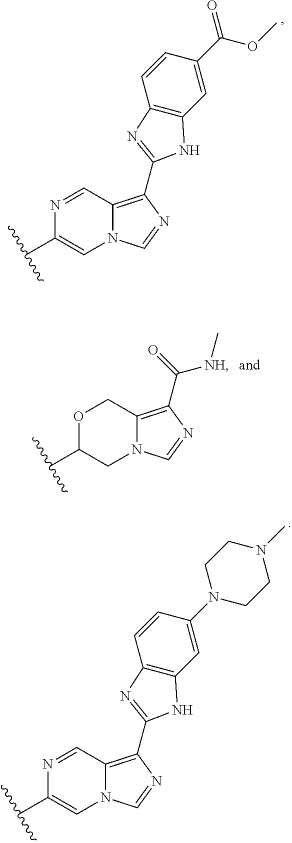 [SMILES: *c1cn2cnc(-c3nc4ccc(C(=O)OC)cc4[nH]3)c2cn1 ; "," ; *C1Cn2cnc(C(=O)NC)c2CO1 ; "and" ; *c1cn2cnc(-c3nc4ccc(N5CCN(C)CC5)cc4[nH]3)c2cn1 ; "."]

In some embodiments, C is selected from the group consisting of

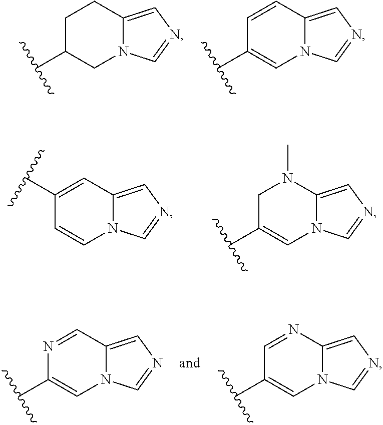

wherein C is substituted with two independent R³ substituents selected from the group consisting of halogen, C₁₋₆alkyl, C₁₋₆alkoxyl, oxo, —C(O)N(R^{A})(R^{B}), —N(R^{A})(R^{B}), 5-10 membered heterocyclyl, and 5-10 membered heteroaryl, wherein the C₁₋₆alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl.

In some embodiments, R³ is independently, for each occurrence, selected from the group consisting of halogen, oxo, C₁₋₆alkyl, C₁₋₆alkoxyl, —C(O)N(R^{A})(R^{B}), —N(R^{A})(R^{B}), and 5-10 membered heteroaryl, wherein the 5-10 membered heteroaryl may be optionally substituted with chloro.

In some embodiments, R^{A} is hydrogen.

In some embodiments, R^{B} is CH₃.

In some embodiments, R³ is selected from the group consisting of fluoro, oxo, CH₃, —O—CH₃, —NHCH₃,

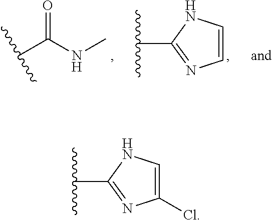

In some embodiments, C is selected from the group consisting of

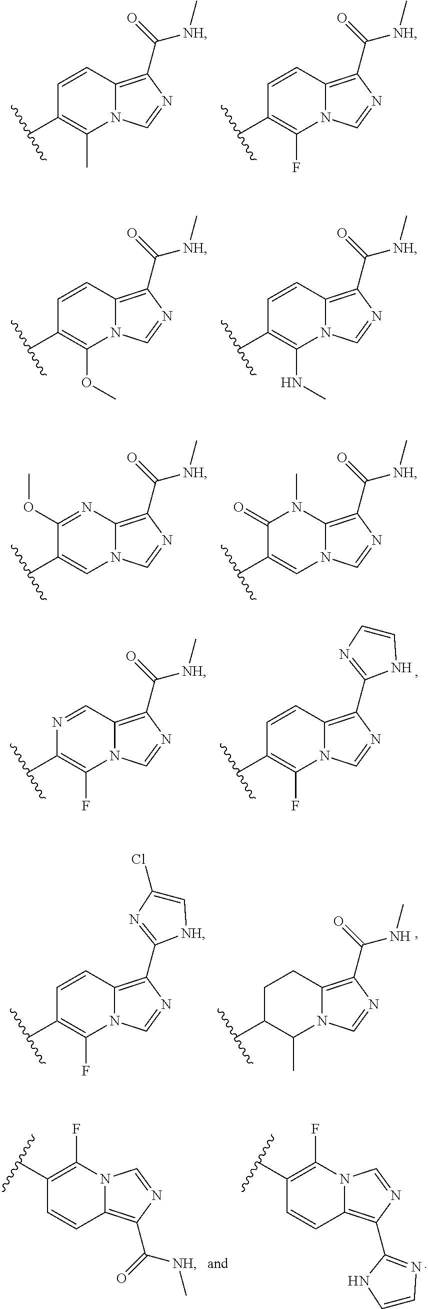

In another aspect, the compound is selected from any compound set forth in Table 1, or a pharmaceutically acceptable salt thereof.

In certain embodiments, the compound is selected from the group consisting of
6-[3-(5-chloro-2-methoxypyridine-3-sulfonamido)-2,6-difluorophenyl]-N-methylimidazo[1,5-a]pyridine-1-carboxamide;
5-chloro-N-[2,4-difluoro-3-[1-(1H-imidazol-2-yl)imidazo[1,5-a]pyridin-6-yl]phenyl]-2-methoxypyridine-3-sulfonamide;
6-[3-(5-chloro-2-methoxypyridine-3-sulfonamido)-2,6-difluorophenyl]-N-methylimidazo[1,5-a]pyrazine-1-carboxamide;
2-[3-(5-Chloro-2-methoxypyridine-3-sulfonamido)-2,6-difluorophenyl]-N-methylimidazo
[1,5-b]pyridazine-5-carboxamide;
2-[3-(5-chloro-2-methylpyridine-3-sulfonamido)-2,6-difluorophenyl]-N-methylimidazo[1,5-b]pyridazine-5-carboxamide;
6-[2,6-difluoro-3-(5-fluoro-2-methylpyridine-3-sulfonamido)phenyl]-N-methylimidazo[1,5-a]pyridine-1-carboxamide;
6-[2,6-difluoro-3-(5-fluoro-2-methoxypyridine-3-sulfonamido)phenyl]-N-methylimidazo[1,5-a]pyridine-1-carboxamide;
6-[3-(5-cyano-2-methoxypyridine-3-sulfonamido)-2,6-difluorophenyl]-N-methylimidazo[1,5-a]pyridine-1-carboxamide;
6-[3-(5-cyano-2-methylpyridine-3-sulfonamido)-2,6-difluorophenyl]-N-methylimidazo[1,5-a]pyridine-1-carboxamide;
6-[2,6-difluoro-3-(6-fluoro-1-hydroxy-2,3-dihydro-1H-indene-4-sulfonamido)phenyl]-N-methylimidazo[1,5-a]pyridine-1-carboxamide;
6-[3-(5-chloro-2-methoxypyridine-3-sulfonamido)-2,6-difluorophenyl]-N,5-dimethyl imidazo[1,5-a]pyridine-1-carboxamide;
2-[2,6-difluoro-3-(5-fluoro-2-methylpyridine-3-sulfonamido)phenyl]-N-methylimidazo[1,5-b]pyridazine-5-carboxamide;
6-[2,6-difluoro-3-[2-methoxy-5-(trifluoromethyl)pyridine-3-sulfonamido]phenyl]-N-methylimidazo[1,5-a]pyridine-1-carboxamide;
6-[3-(5-chloro-2-methoxypyridine-3-sulfonamido)-2,6-difluorophenyl]-5-fluoro-N-methylimidazo[1,5-a]pyridine-1-carboxamide;
6-[3-(5-chloro-2-methoxypyridine-3-sulfonamido)-2,6-difluorophenyl]-5-methoxy-N-methylimidazo[1,5-a]pyridine-1-carboxamide;
3-[3-(5-chloro-2-methoxypyridine-3-sulfonamido)-2,6-difluorophenyl]-N-methylimidazo[1,5-a]pyrimidine-8-carboxamide;
3-[3-(5-chloro-2-methoxypyridine-3-sulfonamido)-2,6-difluorophenyl]-2-methoxy-N-methylimidazo[1,5-a]pyrimidine-8-carboxamide;
N-[2,4-difluoro-3-[1-(1H-imidazol-2-yl)imidazo[1,5-a]pyridin-6-yl]phenyl]-5-fluoro-2-methoxypyridine-3-sulfonamide;
N-[2,4-difluoro-3-[1-(1H-imidazol-2-yl)imidazo[1,5-a]pyridin-6-yl]phenyl]-5-fluoro-2-methylpyridine-3-sulfonamide;
5-cyano-N-[2,4-difluoro-3-[1-(1H-imidazol-2-yl)imidazo[1,5-a]pyridin-6-yl]phenyl]-2-methoxypyridine-3-sulfonamide;
7-[3-(5-chloro-2-methoxypyridine-3-sulfonamido)-2,6-difluorophenyl]-8-fluoro-N-methylimidazo[1,5-a]pyridine-3-carboxamide;
3-[3-(5-chloro-2-methoxypyridine-3-sulfonamido)-2,6-difluorophenyl]-N,1-dimethyl-2-oxoimidazo[1,5-a]pyrimidine-8-carboxamide;
6-[3-[3-cyano-5-(trifluoromethyl)benzenesulfonamido]-2,6-difluorophenyl]-N-methylimidazo[1,5-a]pyridine-1-carboxamide;
6-[3-(5-chloro-2-methoxypyridine-3-sulfonamido)-2,6-difluorophenyl]-N-methyl-5-(methylamino)imidazo[1,5-a]pyridine-1-carboxamide;
5-chloro-N-[2,4-difluoro-3-[1-(1H-imidazol-2-yl)imidazo[1,5-a]pyridin-6-yl]phenyl]-2-methylpyridine-3-sulfonamide;
5-chloro-N-[3,5-difluoro-4-[1-(1H-imidazol-2-yl)imidazo[1,5-a]pyridin-6-yl]pyridin-2-yl]-2-methoxypyridine-3-sulfonamide;
(6R)-6-[3-(5-chloro-2-methoxypyridine-3-sulfonamido)-2,6-difluorophenyl]-N-methyl-5H,6H,7H,8H-imidazo[1,5-a]pyridine-1-carboxamide;
(6S)-6-[3-(5-chloro-2-methoxypyridine-3-sulfonamido)-2,6-difluorophenyl]-N-methyl-5H,6H,7H,8H-imidazo[1,5-a]pyridine-1-carboxamide;
N-[2,4-difluoro-3-[1-(1H-imidazol-2-yl)imidazo[1,5-a]pyridin-6-yl]phenyl]-6-fluoro-1-hydroxy-2,3-dihydro-1H-indene-4-sulfonamide;
5-chloro-N-[2,4-difluoro-3-[1-(4H-1,2,4-triazol-3-yl)imidazo[1,5-a]pyridin-6-yl]phenyl]-2-methoxypyridine-3-sulfonamide;
7-[3-(5-chloro-2-methoxypyridine-3-sulfonamido)-2,6-difluorophenyl]-N-methylimidazo[1,5-a]pyridine-3-carboxamide;
5-chloro-N-[2,4-difluoro-3-[1-(1H-imidazol-2-yl)imidazo[1,5-a]pyrazin-6-yl]phenyl]-2-methoxypyridine-3-sulfonamide;
5-chloro-N-[2,4-difluoro-3-[1-(2H-pyrazol-3-yl)imidazo[1,5-a]pyridin-6-yl]phenyl]-2-methoxypyridine-3-sulfonamide;
(6R)-6-[3-(5-chloro-2-methoxypyridine-3-sulfonamido)-2,6-difluorophenyl]-N,7-dimethyl-5H,6H,8H-imidazo[1,5-a]pyrazine-1-carboxamide;
(6S)-6-[3-(5-chloro-2-methoxypyridine-3-sulfonamido)-2,6-difluorophenyl]-N,7-dimethyl-5H,6H,8H-imidazo[1,5-a]pyrazine-1-carboxamide;
5-chloro-N-[2,4-difluoro-3-[1-(3H-1,2,3-triazol-4-yl)imidazo[1,5-a]pyridin-6-yl]phenyl]-2-methoxypyridine-3-sulfonamide;
5-chloro-N-[2,4-difluoro-3-[3-(1H-imidazol-2-yl)imidazo[1,5-a]pyridin-7-yl]phenyl]-2-methoxypyridine-3-sulfonamide;
N-[2,4-difluoro-3-[3-(1H-imidazol-2-yl)imidazo[1,5-a]pyridin-7-yl]phenyl]-5-fluoro-2-methoxypyridine-3-sulfonamide;
5-chloro-N-[2,4-difluoro-3-[1-(1-methylpyrazol-3-yl)imidazo[1,5-a]pyridin-6-yl]phenyl]-2-methoxypyridine-3-sulfonamide;
5-chloro-N-[2-cyano-4-fluoro-3-[1-(1H-imidazol-2-yl)imidazo[1,5-a]pyridin-6-yl]phenyl]-2-methoxypyridine-3-sulfonamide;
6-cyano-N-[2,4-difluoro-3-[1-(1H-imidazol-2-yl)imidazo[1,5-a]pyridin-6-yl]phenyl]-1-hydroxy-2,3-dihydro-1H-indene-4-sulfonamide;
5-cyano-N-[2,4-difluoro-3-[3-(1H-imidazol-2-yl)imidazo[1,5-a]pyridin-7-yl]phenyl]-2-methoxypyridine-3-sulfonamide;
5-chloro-N-[2,4-difluoro-3-[8-fluoro-3-(1H-imidazol-2-yl)imidazo[1,5-a]pyridin-7-yl]phenyl]-2-methoxypyridine-3-sulfonamide;
5-cyano-N-[2,4-difluoro-3-[8-fluoro-3-(1H-imidazol-2-yl)imidazo[1,5-a]pyridin-7-yl]phenyl]-2-methoxypyridine-3-sulfonamide;
5-chloro-N-(3-(1-(4-cyano-1H-imidazol-2-yl)imidazo[1,5-a]pyridin-6-yl)-2,4-difluorophenyl)-2-methoxypyridine-3-sulfonamide;
N-[3-[1-(4-cyano-1H-imidazol-2-yl)imidazo[1,5-a]pyridin-6-yl]-2,4-difluorophenyl]-5-fluoro-2-methylpyridine-3-sulfonamide;
N-[3-[1-(4-chloro-1H-imidazol-2-yl)imidazo[1,5-a]pyridin-6-yl]-2,4-difluorophenyl]-5-fluoro-2-methoxypyridine-3-sulfonamide;
N-[2,4-difluoro-3-[1-(1-methylpyrazol-3-yl)imidazo[1,5-a]pyridin-6-yl]phenyl]-5-fluoro-2-methoxypyridine-3-sulfonamide;
N-[2,4-difluoro-3-[8-fluoro-3-(1H-imidazol-2-yl)imidazo[1,5-a]pyridin-7-yl]phenyl]-5-fluoro-2-methoxypyridine-3-sulfonamide;
5-chloro-N-[2,4-difluoro-3-[1-(4-methyl-3H-imidazol-2-yl)imidazo[1,5-a]pyridine-6-yl]phenyl]-2-methoxypyridine-3-sulfonamide;
5-chloro-N-(2,4-difluoro-3-[1-[4-(hydroxymethyl)-3H-imidazol-2-yl]imidazo[1,5-a]pyridin-6-yl]phenyl)-2-methoxypyridine-3-sulfonamide;
N-[2,4-difluoro-3-[1-(4-methyl-3H-imidazol-2-yl)imidazo[1,5-a]pyridin-6-yl]phenyl]-5-fluoro-2-methylpyridine-3-sulfonamide;
N-(2,4-difluoro-3-[1-[4-(hydroxymethyl)-3H-imidazol-2-yl]imidazo[1,5-a]pyridin-6-yl]phenyl)-5-fluoro-2-methylpyridine-3-sulfonamide;
5-chloro-N-[3-[1-(4-chloro-1H-imidazol-2-yl)imidazo[1,5-a]pyridin-6-yl]-2,4-difluorophenyl]-2-methoxypyridine-3-sulfonamide;
5-cyano-N-[2,4-difluoro-3-[1-(1-methylpyrazol-3-yl)imidazo[1,5-a]pyridin-6-yl]phenyl]-2-methoxypyridine-3-sulfonamide;
N-[2,4-difluoro-3-[1-(1-methylpyrazol-3-yl)imidazo[1,5-a]pyridin-6-yl]phenyl]-5-fluoro-2-methylpyridine-3-sulfonamide;
5-chloro-N-[2,4-difluoro-3-[1-(1,2,3-triazol-1-yl)imidazo[1,5-a]pyridin-6-yl]phenyl]-2-methoxypyridine-3-sulfonamide;
5-chloro-N-[2,4-difluoro-3-[1-(1,2,3-triazol-2-yl)imidazo[1,5-a]pyridin-6-yl]phenyl]-2-methoxypyridine-3-sulfonamide;
5-chloro-N-[2,4-difluoro-3-[1-(4-methyl-3H-imidazol-2-yl)imidazo[1,5-a]pyridin-6-yl]phenyl]-2-methylpyridine-3-sulfonamide;
5-chloro-N-[2,4-difluoro-3-[1-(5-methyl-4H-1,2,4-triazol-3-yl)imidazo[1,5-a]pyridin-6-yl]phenyl]-2-methoxypyridine-3-sulfonamide;
N-[2,4-difluoro-3-[1-(5-methyl-4H-1,2,4-triazol-3-yl)imidazo[1,5-a]pyridin-6-yl]phenyl]-5-fluoro-2-methoxypyridine-3-sulfonamide;
5-cyano-N-[2,4-difluoro-3-[1-(5-methyl-4H-1,2,4-triazol-3-yl)imidazo[1,5-a]pyridin-6-yl]phenyl]-2-methoxypyridine-3-sulfonamide;
N-[2,4-difluoro-3-[1-(5-methyl-4H-1,2,4-triazol-3-yl)imidazo[1,5-a]pyridin-6-yl]phenyl]-5-fluoro-2-methylpyridine-3-sulfonamide;
5-chloro-N-[2,4-difluoro-3-[1-(5-methyl-4H-1,2,4-triazol-3-yl)imidazo[1,5-a]pyridin-6-yl]phenyl]-2-methylpyridine-3-sulfonamide;
5-chloro-N-(2,4-difluoro-3-(1-(1-methyl-1H-pyrazol-3-yl)imidazo[1,5-a]pyridin-6-yl)phenyl)-2-methylpyridine-3-sulfonamide;
5-chloro-N-[2,4-difluoro-3-[1-(1,2,3,4-tetrazol-1-yl)imidazo[1,5-a]pyridin-6-yl]phenyl]-2-methoxypyridine-3-sulfonamide;
(R)—N-(3-(3-(1H-imidazol-2-yl)-5,6,7,8-tetrahydroimidazo[1,5-a]pyridin-7-yl)-2,4-difluorophenyl)-5-chloro-2-methoxypyridine-3-sulfonamide;
(S)—N-(3-(3-(1H-imidazol-2-yl)-5,6,7,8-tetrahydroimidazo[1,5-a]pyridin-7-yl)-2,4-difluorophenyl)-5-chloro-2-methoxypyridine-3-sulfonamide;
5-chloro-N-[2,4-difluoro-3-[1-(4H-1,2,4-triazol-3-yl)imidazo[1,5-a]pyridin-6-yl]phenyl]-2-methylpyridine-3-sulfonamide;
N-[3-[1-(1H-1,3-benzodiazol-2-yl)imidazo[1,5-a]pyridin-6-yl]-2,4-difluorophenyl]-5-cyano-2-methoxypyridine-3-sulfonamide;
(R)-6-(2,6-difluoro-3-((5-fluoro-2-methoxypyridine)-3-sulfonamido)phenyl)-N-methyl-5,6,7,8-tetrahydroimidazo[1,5-a]pyridine-1-carboxamide;
(S)-6-(2,6-difluoro-3-((5-fluoro-2-methoxypyridine)-3-sulfonamido)phenyl)-N-methyl-5,6,7,8-tetrahydroimidazo[1,5-a]pyridine-1-carboxamide;
(R)-6-(3-((5-cyano-2-methoxypyridine)-3-sulfonamido)-2,6-difluorophenyl)-N-methyl-5,6,7,8-tetrahydroimidazo[1,5-a]pyridine-1-carboxamide;
(S)-6-(3-((5-cyano-2-methoxypyridine)-3-sulfonamido)-2,6-difluorophenyl)-N-methyl-5,6,7,8-tetrahydroimidazo[1,5-a]pyridine-1-carboxamide;
5-cyano-N-[2,4-difluoro-3-[1-(1H-imidazol-2-yl)imidazo[1,5-a]pyrazin-6-yl]phenyl]-2-methoxypyridine-3-sulfonamide;
N-[2,4-difluoro-3-[1-(4H-1,2,4-triazol-3-yl)imidazo[1,5-a]pyridin-6-yl]phenyl]-5-fluoro-2-methoxypyridine-3-sulfonamide;
5-cyano-N-[2,4-difluoro-3-[1-(4H-1,2,4-triazol-3-yl)imidazo[1,5-a]pyridin-6-yl]phenyl]-2-methoxypyridine-3-sulfonamide;
N-[2,4-difluoro-3-[1-(4H-1,2,4-triazol-3-yl)imidazo[1,5-a]pyridin-6-yl]phenyl]-5-fluoro-2-methylpyridine-3-sulfonamide;
5-chloro-N-[2,4-difluoro-3-[(6R)-1-(1H-imidazol-2-yl)-7-methyl-5H,6H,8H-imidazo[1,5-a]pyrazin-6-yl]phenyl]-2-methoxypyridine-3-sulfonamide;
5-chloro-N-[2,4-difluoro-3-[(6S)-1-(1H-imidazol-2-yl)-7-methyl-5H,6H,8H-imidazo[1,5-a]pyrazin-6-yl]phenyl]-2-methoxypyridine-3-sulfonamide;
N-[2,4-difluoro-3-[1-(1,2,3,4-tetrazol-1-yl)imidazo[1,5-a]pyridin-6-yl]phenyl]-5-fluoro-2-methoxypyridine-3-sulfonamide;
5-cyano-N-[2,4-difluoro-3-[1-(1,2,3,4-tetrazol-1-yl)imidazo[1,5-a]pyridin-6-yl]phenyl]-2-methoxypyridine-3-sulfonamide;
N-[2,4-difluoro-3-[1-(1,2,3,4-tetrazol-1-yl)imidazo[1,5-a]pyridin-6-yl]phenyl]-5-fluoro-2-methylpyridine-3-sulfonamide;
5-chloro-N-[2,4-difluoro-3-[1-(1,2,3,4-tetrazol-1-yl)imidazo[1,5-a]pyridin-6-yl]phenyl]-2-methylpyridine-3-sulfonamide;
N-[2,4-difluoro-3-[1-(1H-imidazol-2-yl)imidazo[1,5-a]pyrazin-6-yl]phenyl]-5-fluoro-2-methoxypyridine-3-sulfonamide;
5-chloro-N-[2,4-difluoro-3-[(6R)-1-(1H-imidazol-2-yl)-5H,6H,7H,8H-imidazo[1,5-a]pyridin-6-yl]phenyl]-2-methoxypyridine-3-sulfonamide;
5-chloro-N-[2,4-difluoro-3-[(6S)-1-(1H-imidazol-2-yl)-5H,6H,7H,8H-imidazo[1,5-a]pyridin-6-yl]phenyl]-2-methoxypyridine-3-sulfonamide;
N-[2,4-difluoro-3-[(6R)-1-(1H-imidazol-2-yl)-5H,6H,7H,8H-imidazo[1,5-a]pyridin-6-yl]phenyl]-5-fluoro-2-methylpyridine-3-sulfonamide;
N-[2,4-difluoro-3-[(6S)-1-(1H-imidazol-2-yl)-5H,6H,7H,8H-imidazo[1,5-a]pyridin-6-yl]phenyl]-5-fluoro-2-methylpyridine-3-sulfonamide;
N-[2,4-difluoro-3-[(6R)-1-(1H-imidazol-2-yl)-5H,6H,7H,8H-imidazo[1,5-a]pyridin-6-yl]phenyl]-5-fluoro-2-methoxypyridine-3-sulfonamide;
N-[2,4-difluoro-3-[(6S)-1-(1H-imidazol-2-yl)-5H,6H,7H,8H-imidazo[1,5-a]pyridin-6-yl]phenyl]-5-fluoro-2-methoxypyridine-3-sulfonamide;
5-cyano-N-[2,4-difluoro-3-[(6R)-1-(1H-imidazol-2-yl)-5H,6H,7H,8H-imidazo[1,5-a]pyridin-6-yl]phenyl]-2-methoxypyridine-3-sulfonamide;
5-cyano-N-[2,4-difluoro-3-[(6S)-1-(1H-imidazol-2-yl)-5H,6H,7H,8H-imidazo[1,5-a]pyridin-6-yl]phenyl]-2-methoxypyridine-3-sulfonamide;
5-chloro-N-[2,4-difluoro-3-[1-(1H-imidazol-2-yl)imidazo[1,5-a]pyrazin-6-yl]phenyl]-2-methylpyridine-3-sulfonamide;
5-chloro-N-[2,4-difluoro-3-[(6R)-1-(1H-imidazol-2-yl)-5H,6H,7H,8H-imidazo[1,5-a]pyridin-6-yl]phenyl]-2-methylpyridine-3-sulfonamide;
5-chloro-N-[2,4-difluoro-3-[(6S)-1-(1H-imidazol-2-yl)-5H,6H,7H,8H-imidazo[1,5-a]pyridin-6-yl]phenyl]-2-methylpyridine-3-sulfonamide;
5-chloro-N-[2,4-difluoro-3-[5-(1H-imidazol-2-yl)imidazo[1,5-b]pyridazin-2-yl]phenyl]-2-methoxypyridine-3-sulfonamide;
(R)-6-(2,6-difluoro-3-((5-fluoro-2-methylpyridine)-3-sulfonamido)phenyl)-N-methyl-5,6,7,8-tetrahydroimidazo[1,5-a]pyridine-1-carboxamide;
(S)-6-(2,6-difluoro-3-((5-fluoro-2-methylpyridine)-3-sulfonamido)phenyl)-N-methyl-5,6,7,8-tetrahydroimidazo[1,5-a]pyridine-1-carboxamide;
5-chloro-N-[3-[1-(5-cyclopropyl-4H-1,2,4-triazol-3-yl)imidazo[1,5-a]pyridin-6-yl]-2,4-difluorophenyl]-2-methoxypyridine-3-sulfonamide;
5-chloro-N-[2,4-difluoro-3-[1-(5-isopropyl-4H-1,2,4-triazol-3-yl)imidazo[1,5-a]pyridin-6-yl]phenyl]-2-methoxypyridine-3-sulfonamide;
(R)-6-(3-((5-chloro-2-methylpyridine)-3-sulfonamido)-2,6-difluorophenyl)-N-methyl-5,6,7,8-tetrahydroimidazo[1,5-a]pyridine-1-carboxamide;
(S)-6-(3-((5-chloro-2-methylpyridine)-3-sulfonamido)-2,6-difluorophenyl)-N-methyl-5,6,7,8-tetrahydroimidazo[1,5-a]pyridine-1-carboxamide;
6-[2,6-difluoro-3-(5-fluoro-2-methoxypyridine-3-sulfonamido)phenyl]-N-methylimidazo
[1,5-a]pyrazine-1-carboxamide;
6-[2,6-difluoro-3-(5-fluoro-2-methylpyridine-3-sulfonamido)phenyl]-N-methylimidazo[1,5-a]pyrazine-1-carboxamide;
6-[3-(5-cyano-2-methoxypyridine-3-sulfonamido)-2,6-difluorophenyl]-N-methylimidazo[1,5-a]pyrazine-1-carboxamide;
6-[3-(5-chloro-2-methylpyridine-3-sulfonamido)-2,6-difluorophenyl]-N-methylimidazo[1,5-a]pyrazine-1-carboxamide;
5-chloro-N-(2,4-difluoro-3-(1-(hydroxymethyl)imidazo[1,5-a]pyridin-6-yl)phenyl)-2-methoxypyridine-3-sulfonamide;
5-chloro-N-[2,4-difluoro-3-[5-(1H-imidazol-2-yl)imidazo[1,5-b]pyridazin-2-yl]phenyl]-2-methylpyridine-3-sulfonamide;
(5S,6R)-6-[3-(5-chloro-2-methoxypyridine-3-sulfonamido)-2,6-difluorophenyl]-N,5-dimethyl-5H,6H,7H,8H-imidazo[1,5-a]pyridine-1-carboxamide;
(5R,6S)-6-[3-(5-chloro-2-methoxypyridine-3-sulfonamido)-2,6-difluorophenyl]-N,5-dimethyl-5H,6H,7H,8H-imidazo[1,5-a]pyridine-1-carboxamide;
5-cyano-N-[2,4-difluoro-3-[5-(1H-imidazol-2-yl)imidazo[1,5-b]pyridazin-2-yl]phenyl]-2-methoxypyridine-3-sulfonamide;
N-[2,4-difluoro-3-[5-(1H-imidazol-2-yl)imidazo[1,5-b]pyridazin-2-yl]phenyl]-5-fluoro-2-methoxypyridine-3-sulfonamide;
5-chloro-N-(2,4-difluoro-3-(1-(isoxazol-5-yl)imidazo[1,5-a]pyridin-6-yl)phenyl)-2-methoxypyridine-3-sulfonamide;
5-chloro-N-[2,4-difluoro-3-[1-(1-hydroxyethyl)imidazo[1,5-a]pyridin-6-yl]phenyl]-2-methoxypyridine-3-sulfonamide;
6-(2-chloro-3-((5-chloro-2-methoxypyridine)-3-sulfonamido)-6-fluorophenyl)-N-methylimidazo[1,5-a]pyridine-1-carboxamide;
5-chloro-N-(2,4-difluoro-3-[1-[5-(trifluoromethyl)-4H-1,2,4-triazol-3-yl]imidazo[1,5-a]pyridin-6-yl]phenyl)-2-methoxypyridine-3-sulfonamide;
5-chloro-N-[2-chloro-4-fluoro-3-[1-(1H-imidazol-2-yl)imidazo[1,5-a]pyridin-6-yl]phenyl]-2-methoxypyridine-3-sulfonamide;
5-chloro-N-[2,4-difluoro-3-[5-fluoro-1-(1H-imidazol-2-yl)imidazo[1,5-a]pyridin-6-yl]phenyl]-2-methoxypyridine-3-sulfonamide;
5-chloro-N-[2,4-difluoro-3-[5-fluoro-1-(2H-pyrazol-3-yl)imidazo[1,5-a]pyridin-6-yl]phenyl]-2-methoxypyridine-3-sulfonamide;
6-[2-chloro-3-(5-chloro-2-methoxypyridine-3-sulfonamido)-6-fluorophenyl]-N-methylimidazo[1,5-a]pyrazine-1-carboxamide;
N-[2,4-difluoro-3-[5-fluoro-1-(1H-imidazol-2-yl)imidazo[1,5-a]pyridin-6-yl]phenyl]-5-fluoro-2-methoxypyridine-3-sulfonamide;
N-[2,4-difluoro-3-[5-fluoro-1-(1H-imidazol-2-yl)imidazo[1,5-a]pyridin-6-yl]phenyl]-5-fluoro-2-methylpyridine-3-sulfonamide;
5-chloro-N-[2,4-difluoro-3-[5-fluoro-1-(1H-imidazol-2-yl)imidazo[1,5-a]pyridine-6-yl]phenyl]-2-methylpyridine-3-sulfonamide;
N-[3-[1-(1H-1,3-benzodiazol-2-yl)imidazo[1,5-a]pyrazin-6-yl]-2,4-difluorophenyl]-5-chloro-2-methoxypyridine-3-sulfonamide;
N-[3-[1-(1H-1,3-benzodiazol-2-yl)imidazo[1,5-a]pyrazin-6-yl]-2,4-difluorophenyl]-5-fluoro-2-methoxypyridine-3-sulfonamide;
N-[3-[1-(1H-1,3-benzodiazol-2-yl)imidazo[1,5-a]pyrazin-6-yl]-2,4-difluorophenyl]-5-cyano-2-methoxypyridine-3-sulfonamide;
N-[3-[1-(1H-1,3-benzodiazol-2-yl)imidazo[1,5-a]pyrazin-6-yl]-2,4-difluorophenyl]-5-chloro-2-methylpyridine-3-sulfonamide;
N-[3-[1-(1H-1,3-benzodiazol-2-yl)imidazo[1,5-a]pyrazin-6-yl]-2,4-difluorophenyl]-5-fluoro-2-methylpyridine-3-sulfonamide;
N-[3-[1-(4-chloro-1H-imidazol-2-yl)-5-fluoroimidazo[1,5-a]pyridin-6-yl]-2,4-difluorophenyl]-5-fluoro-2-methoxypyridine-3-sulfonamide;
5-chloro-N-[3-[1-(4-chloro-1H-imidazol-2-yl)-5-fluoroimidazo[1,5-a]pyridin-6-yl]-2,4-difluorophenyl]-2-methoxypyridine-3-sulfonamide;
6-[3-(5-chloro-2-methoxypyridine-3-sulfonamido)-2-cyano-6-fluorophenyl]-N-methylimidazo[1,5-a]pyrazine-1-carboxamide;
N-[2,4-difluoro-3-[1-(1,2-oxazol-5-yl)imidazo[1,5-a]pyridin-6-yl]phenyl]-5-fluoro-2-methoxypyridine-3-sulfonamide;
5-cyano-N-[2,4-difluoro-3-[1-(1,2-oxazol-5-yl)imidazo[1,5-a]pyridin-6-yl]phenyl]-2-methoxypyridine-3-sulfonamide;
2-[3-(5-chloro-2-methoxypyridine-3-sulfonamido)-2,6-difluorophenyl]-N-methylimidazo[4,3-b][1,3]thiazole-7-carboxamide;
5-chloro-N-[2,4-difluoro-3-[1-(1,2-oxazol-5-yl)imidazo[1,5-a]pyridin-6-yl]phenyl]-2-methylpyridine-3-sulfonamide;
6-[3-(5-chloro-2-methoxypyridine-3-sulfonamido)-6-fluoro-2-methylphenyl]-N-methylimidazo[1,5-a]pyridine-1-carboxamide;
5-cyano-N-[2,4-difluoro-3-[1-(4,5,6,7-tetrahydro-1H-1,3-benzodiazol-2-yl)imidazo[1,5-a]pyridin-6-yl]phenyl]-2-methoxypyridine-3-sulfonamide;
5-chloro-N-[2,4-difluoro-3-[1-(5-methyl-4H-1,2,4-triazol-3-yl)imidazo[1,5-a]pyrazin-6-yl]phenyl]-2-methoxypyridine-3-sulfonamide;
N-[2,4-difluoro-3-[1-(1,2-oxazol-5-yl)imidazo[1,5-a]pyridin-6-yl]phenyl]-5-fluoro-2-methylpyridine-3-sulfonamide;
5-cyano-N-[3-[1-(4,5-dimethyl-1H-imidazol-2-yl)imidazo[1,5-a]pyridin-6-yl]-2,4-difluorophenyl]-2-methoxypyridine-3-sulfonamide;
N-[2,4-difluoro-3-[1-(5-methyl-4H-1,2,4-triazol-3-yl)imidazo[1,5-a]pyrazin-6-yl]phenyl]-5-fluoro-2-methoxypyridine-3-sulfonamide;
5-cyano-N-[2,4-difluoro-3-[1-(5-methyl-4H-1,2,4-triazol-3-yl)imidazo[1,5-a]pyrazin-6-yl]phenyl]-2-methoxypyridine-3-sulfonamide;
N-[2,4-difluoro-3-[1-(5-methyl-4H-1,2,4-triazol-3-yl)imidazo[1,5-a]pyrazin-6-yl]phenyl]-5-fluoro-2-methylpyridine-3-sulfonamide;
5-chloro-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methoxypyridine-3-sulfonamide;
5-chloro-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methoxypyridine-3-sulfonamide;
N-[2,4-difluoro-3-[(6S)-1-(5-methyl-4H-1,2,4-triazol-3-yl)-5H,6H,7H,8H-imidazo[1,5-a]pyridin-6-yl]phenyl]-5-fluoro-2-methoxypyridine-3-sulfonamide;
N-[2,4-difluoro-3-[(6R)-1-(5-methyl-4H-1,2,4-triazol-3-yl)-5H,6H,7H,8H-imidazo[1,5-a]pyridin-6-yl]phenyl]-5-fluoro-2-methoxypyridine-3-sulfonamide;
6-(3-((5-chloro-2-oxo-1,2-dihydropyridine)-3-sulfonamido)-2,6-difluorophenyl)-N-methylimidazo[1,5-a]pyrazine-1-carboxamide;
5-chloro-N-[2,4-difluoro-3-[1-(5-methyl-4H-1,2,4-triazol-3-yl)imidazo[1,5-a]pyrazin-6-yl]phenyl]-2-methylpyridine-3-sulfonamide;
N-[2,4-difluoro-3-[(6S)-1-(5-methyl-4H-1,2,4-triazol-3-yl)-5H,6H,7H,8H-imidazo[1,5-a]pyridin-6-yl]phenyl]-5-fluoro-2-methylpyridine-3-sulfonamide;
N-[2,4-difluoro-3-[(6R)-1-(5-methyl-4H-1,2,4-triazol-3-yl)-5H,6H,7H,8H-imidazo[1,5-a]pyridin-6-yl]phenyl]-5-fluoro-2-methylpyridine-3-sulfonamide;
5-cyano-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methoxypyridine-3-sulfonamide;
5-cyano-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methoxypyridine-3-sulfonamide;
5-chloro-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methylpyridine-3-sulfonamide;
5-chloro-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methylpyridine-3-sulfonamide;
6-[6-chloro-3-(5-chloro-2-methoxypyridine-3-sulfonamido)-2-fluorophenyl]-N-methylimidazo[1,5-a]pyridine-1-carboxamide;
2-[6-chloro-3-(5-chloro-2-methylpyridine-3-sulfonamido)-2-fluorophenyl]-N-methylimidazo[1,5-b]pyridazine-5-carboxamide;
2-[2-chloro-3-(5-chloro-2-methylpyridine-3-sulfonamido)-6-fluorophenyl]-N-methylimidazo[1,5-b]pyridazine-5-carboxamide;
6-(2-fluoro-5-((5-fluoro-2-methoxypyridine)-3-sulfonamido)phenyl)-N-methyl-5,6,7,8-tetrahydroimidazo[1,5-a]pyridine-1-carboxamide;
6-(6-chloro-3-((5-chloro-2-methoxypyridine)-3-sulfonamido)-2-fluorophenyl)-N-methylimidazo[1,5-a]pyrazine-1-carboxamide;
N-[4-chloro-2-fluoro-3-[1-(1H-imidazol-2-yl)imidazo[1,5-a]pyrazin-6-yl]phenyl]-5-fluoro-2-methoxypyridine-3-sulfonamide;
5-chloro-N-(3-(1-(4,5-dimethyl-1H-imidazol-2-yl)imidazo[1,5-a]pyridin-6-yl)-2,4-difluorophenyl)-2-methoxypyridine-3-sulfonamide;
N-(3-(1-(4,5-dimethyl-1H-imidazol-2-yl)imidazo[1,5-a]pyridin-6-yl)-2,4-difluorophenyl)-5-fluoro-2-methoxypyridine-3-sulfonamide;
N-(3-(1-(4,5-dimethyl-1H-imidazol-2-yl)imidazo[1,5-a]pyridin-6-yl)-2,4-difluorophenyl)-5-fluoro-2-methylpyridine-3-sulfonamide;
5-chloro-N-(3-(1-(4,5-dimethyl-1H-imidazol-2-yl)imidazo[1,5-a]pyridin-6-yl)-2,4-difluorophenyl)-2-methylpyridine-3-sulfonamide;
5-chloro-N-(2,4-difluoro-3-(1-(4,5,6,7-tetrahydro-1H-benzo[d]imidazol-2-yl) imidazo[1,5-a]pyridin-6-yl)phenyl)-2-methoxypyridine-3-sulfonamide;
N-(2,4-difluoro-3-(1-(4,5,6,7-tetrahydro-1H-benzo[d]imidazol-2-yl)imidazo[1,5-a]pyridin-6-yl)phenyl)-5-fluoro-2-methoxypyridine-3-sulfonamide;
N-(2,4-difluoro-3-(1-(4,5,6,7-tetrahydro-1H-benzo[d]imidazol-2-yl) imidazo[1,5-a]pyridin-6-yl)phenyl)-5-fluoro-2-methylpyridine-3-sulfonamide;
5-chloro-N-(2,4-difluoro-3-(1-(4,5,6,7-tetrahydro-1H-benzo[d]imidazol-2-yl)imidazo[1,5-a]pyridin-6-yl)phenyl)-2-methylpyridine-3-sulfonamide;
N-[2-chloro-4-fluoro-3-[1-(1H-imidazol-2-yl)imidazo[1,5-a]pyrazin-6-yl]phenyl]-5-fluoro-2-methoxypyridine-3-sulfonamide;
(6R)-6-[2-chloro-6-fluoro-3-(5-fluoro-2-methoxypyridine-3-sulfonamido)phenyl]-N-methyl-5H,6H,7H,8H-imidazo[1,5-a]pyridine-1-carboxamide;
(6S)-6-[2-chloro-6-fluoro-3-(5-fluoro-2-methoxypyridine-3-sulfonamido)phenyl]-N-methyl-5H,6H,7H,8H-imidazo[1,5-a]pyridine-1-carboxamide;
methyl 2-[6-[2,6-difluoro-3-(5-fluoro-2-methoxypyridine-3-sulfonamido)phenyl]imidazo[1,5-a]pyrazin-1-yl]-3H-1,3-benzodiazole-5-carboxylate;
(6S)-6-[3-(5-chloro-2-methoxypyridine-3-sulfonamido)-2,6-difluorophenyl]-N-methyl-5H,6H,8H-imidazo[4,3-c][1,4]oxazine-1-carboxamide;
(6R)-6-[3-(5-chloro-2-methoxypyridine-3-sulfonamido)-2,6-difluorophenyl]-N-methyl-5H,6H,8H-imidazo[4,3-c][1,4]oxazine-1-carboxamide;
(6R)-6-[6-chloro-2-fluoro-3-(5-fluoro-2-methoxypyridine-3-sulfonamido)phenyl]-N-methyl-5H,6H,7H,8H-imidazo[1,5-a]pyridine-1-carboxamide;
(6S)-6-[6-chloro-2-fluoro-3-(5-fluoro-2-methoxypyridine-3-sulfonamido)phenyl]-N-methyl-5H,6H,7H,8H-imidazo[1,5-a]pyridine-1-carboxamide;
N-[2,4-difluoro-3-[1-(4-phenyl-1H-imidazol-2-yl)imidazo[1,5-a]pyrazin-6-yl]phenyl]-5-fluoro-2-methoxypyridine-3-sulfonamide;
6-[3-(5-chloro-2-methoxypyridine-3-sulfonamido)-2,6-difluorophenyl]-N-isopropylimidazo[1,5-a]pyrazine-1-carboxamide;
6-[3-(5-chloro-2-methoxypyridine-3-sulfonamido)-2,6-difluorophenyl]-N-(2-methylpropyl)imidazo[1,5-a]pyrazine-1-carboxamide;
6-[3-(5-chloro-2-methoxypyridine-3-sulfonamido)-2,6-difluorophenyl]-N-(2,2,2-trifluoroethyl)imidazo[1,5-a]pyrazine-1-carboxamide;
6-[3-(5-chloro-2-methoxypyridine-3-sulfonamido)-2,6-difluorophenyl]-5-fluoro-N-methylimidazo[1,5-a]pyrazine-1-carboxamide;
6-[3-(5-chloro-2-methoxypyridine-3-sulfonamido)-2,6-difluorophenyl]-N-(2-hydroxyethyl)imidazo[1,5-a]pyrazine-1-carboxamide;
6-[3-(5-chloro-2-methoxypyridine-3-sulfonamido)-2,6-difluorophenyl]-N-(1-methylpyrazol-4-yl)imidazo[1,5-a]pyrazine-1-carboxamide;
6-[3-(5-chloro-2-methoxypyridine-3-sulfonamido)-2,6-difluorophenyl]-N-(oxan-4-ylmethyl)imidazo[1,5-a]pyrazine-1-carboxamide;
6-[3-(5-chloro-2-methoxypyridine-3-sulfonamido)-2,6-difluorophenyl]-N-(2-methanesulfonylethyl)imidazo[1,5-a]pyrazine-1-carboxamide;
6-[3-(5-chloro-2-methoxypyridine-3-sulfonamido)-2,6-difluorophenyl]-N-ethylimidazo[1,5-a]pyrazine-1-carboxamide;
6-[3-(5-chloro-2-methoxypyridine-3-sulfonamido)-2,6-difluorophenyl]-N-(cyclopropylmethyl)imidazo[1,5-a]pyrazine-1-carboxamide;
6-[3-(5-chloro-2-ethoxypyridine-3-sulfonamido)-2,6-difluorophenyl]-N-methylimidazo[1,5-a]pyrazine-1-carboxamide;
6-[3-(5-chloro-2-isopropoxypyridine-3-sulfonamido)-2,6-difluorophenyl]-N-methylimidazo[1,5-a]pyrazine-1-carboxamide;
6-[3-(5-chloro-2-cyclopropoxypyridine-3-sulfonamido)-2,6-difluorophenyl]-N-methylimidazo[1,5-a]pyrazine-1-carboxamide;
6-[2,6-difluoro-3-(5-fluoro-2-methoxypyridine-3-sulfonamido)phenyl]-5-fluoro-N-methylimidazo[1,5-a]pyrazine-1-carboxamide;
6-[3-(5-chloro-2-methoxypyridine-3-sulfonamido)-2,6-difluorophenyl]imidazo[1,5-a]pyrazine-1-carboxamide;
6-[3-(5-chloro-2-methoxypyridine-3-sulfonamido)-2,6-difluorophenyl]-N-[3-(dimethylamino)propyl]imidazo[1,5-a]pyrazine-1-carboxamide;
(R)-6-(3-((5-chloro-2-methoxypyridine)-3-sulfonamido)-2,6-difluorophenyl)-N-(1-methylpyrrolidin-3-yl)imidazo[1,5-a]pyrazine-1-carboxamide;
(S)-6-(3-((5-chloro-2-methoxypyridine)-3-sulfonamido)-2,6-difluorophenyl)-N-(1-methylpyrrolidin-3-yl)imidazo[1,5-a]pyrazine-1-carboxamide;
6-[3-(5-chloro-2-methoxypyridine-3-sulfonamido)-2,6-difluorophenyl]-N-(1,2-oxazol-4-yl)imidazo[1,5-a]pyrazine-1-carboxamide;
6-[3-(5-chloro-2-methoxypyridine-3-sulfonamido)-2,6-difluorophenyl]-N-[2-(morpholin-4-yl)ethyl]imidazo[1,5-a]pyrazine-1-carboxamide;
6-[3-[5-chloro-2-(2-methylpropoxy)pyridine-3-sulfonamido]-2,6-difluorophenyl]-N-methylimidazo[1,5-a]pyrazine-1-carboxamide;
N-[3-[1-(5-cyclopropyl-3H-imidazol-4-yl)imidazo[1,5-a]pyrazin-6-yl]-2,4-difluorophenyl]-5-fluoro-2-methoxypyridine-3-sulfonamide;
N-[3-[1-(4-benzyl-1H-imidazol-2-yl)imidazo[1,5-a]pyrazin-6-yl]-2,4-difluorophenyl]-5-fluoro-2-methoxypyridine-3-sulfonamide;
6-[3-(5-chloro-2-methoxypyridine-3-sulfonamido)-2,6-difluorophenyl]-N-[2-(dimethylamino)ethyl]imidazo[1,5-a]pyrazine-1-carboxamide;
6-[3-(5-chloro-2-methoxypyridine-3-sulfonamido)-2,6-difluorophenyl]-N-[2-(2-oxopyrrolidin-1-yl)ethyl]imidazo[1,5-a]pyrazine-1-carboxamide;
6-[3-(5-chloro-2-methoxypyridine-3-sulfonamido)-2,6-difluorophenyl]-N-[4-[(dimethylamino)methyl]phenyl]imidazo[1,5-a]pyrazine-1-carboxamide;
6-[3-[5-chloro-2-(2,2,2-trifluoroethoxy)pyridine-3-sulfonamido]-2,6-difluorophenyl]-N-methylimidazo[1,5-a]pyrazine-1-carboxamide;
6-[3-[2-(benzyloxy)-5-chloropyridine-3-sulfonamido]-2,6-difluorophenyl]-N-methylimidazo[1,5-a]pyrazine-1-carboxamide;
6-[2,6-difluoro-3-(5-fluoro-2-methylpyridine-3-sulfonamido)phenyl]-5-fluoro-N-methylimidazo[1,5-a]pyrazine-1-carboxamide;
N-(2,4-difluoro-3-[1-[5-(4-methylpiperazin-1-yl)-3H-1,3-benzodiazol-2-yl]imidazo[1,5-a]pyridin-6-yl]phenyl)-5-fluoro-2-methoxypyridine-3-sulfonamide; and
6-[3-(5-chloro-2-methylpyridine-3-sulfonamido)-2,6-difluorophenyl]-5-fluoro-N-methylimidazo[1,5-a]pyrazine-1-carboxamide;
   or a pharmaceutically acceptable salt thereof.

### Therapeutic Applications GCN2 Modulating (Inhibiting/Activating) Compounds

It is contemplated that GCN2 modulating (inhibiting/activating) compounds and related compounds described herein, such as a compound of Formula I, provide therapeutic benefits to subjects suffering from cancer, neurodegenerative disease, and doxorubicin-induced cardiotoxicity. Accordingly, one aspect of the invention provides therapeutic methods for treating the foregoing diseases and conditions using GCN2 modulating (inhibiting/activating) compounds and related compounds described herein. Various aspects and embodiments of the therapeutic methods are described below.

### Cancer

One aspect of the invention provides a method of treating cancer in a subject. The method comprises administering a therapeutically effective amount of a GCN2 modulating (inhibiting/activating) compound or related compound described herein, such as a compound of Formula 1 to a subject in need thereof to treat the cancer. In certain embodiments, the particular compound of Formula I, is a compound defined by one of the embodiments described above.

In certain embodiments, the cancer is a solid tumor, leukemia, or lymphoma. In certain embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, or lymphoma. In certain embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, leukemia, or lymphoma. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, esophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

Additional exemplary cancers include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma.

In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, or lymphoma.

### Neurodegenerative Disease

Another aspect of the invention provides a method of treating a neurodegenerative disease in a subject. The method comprises administering a therapeutically effective amount of a compound described herein, such as a compound of Formula I, to a subject in need thereof to treat the neurodegenerative disease. In certain embodiments, the neurodegenerative disease is Alzheimer's disease, Parkinson's Disease, Huntington's Disease, amyotrophic lateral sclerosis, or spinocerebellar ataxia.

Aberrant autophagic processes contribute to neurodegenerative diseases. For example, γ-secretase activity is enhanced in autophagic vacuoles through signal transduction mediated by GCN2 phosphorylation of the a subunit of eukaryotic initiation factor 2 (eIF2α) (see, e.g., Ohta, K. et al. in Autophagy 2010, 6, 345-352). The γ-secretase enhances amyloid-R synthesis and the progression of Alzheimer's disease. Accordingly, compounds having inhibitory activity towards GCN2 provide benefits to patients suffering from neurodegenerative diseases.

In certain embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, leukemia, or lymphoma.

### Doxorubicin-Induced Cardiotoxicity

Another aspect of the invention provides a method of treating doxorubicin-induced cardiotoxicity in a subject. The method comprises administering a therapeutically effective amount of a compound described herein, such as a compound of Formula I, to a subject in need thereof suffering from doxorubicin-induced cardiotoxicity, to thereby treat the doxorubicin-induced cardiotoxicity.

Another aspect of the invention provides a method of preventing doxorubicin-induced cardiotoxicity in a subject. The method comprises administering a therapeutically effective amount of a compound described herein, such as a compound of Formula I, to a subject in need thereof that has received, or will receive, doxorubicin, to thereby prevent doxorubicin-induced cardiotoxicity.

Deficiency in GCN2 has been reported to ameliorate doxorubicin-induced cardiotoxicity. See, for example, Wang et al. in Redox Biology (2018) vol. 17, pages 25-34. Accordingly, compounds having inhibitory activity towards GCN2 provide benefits to patients suffering from or likely to suffer from doxorubicin-induced cardiotoxicity.

In certain embodiments, the subject is a human.

Another aspect of the invention provides for the use of a compound described herein (such as a compound of Formula I) in the manufacture of a medicament. In certain embodiments, the medicament is for treating a disorder described herein, such as cancer.

Another aspect of the invention provides for the use of a compound described herein (such as a compound of Formula I) for treating a medical disorder, such a medical disorder described herein (e.g., cancer).

Further, it is contemplated that GCN2 modulators (inhibitors/activators) and related compounds described herein, such as a compound of Formula I, can inhibit/activate the activity of GCN2. Accordingly, another aspect of the invention provides a method of inhibiting/activating the activity of GCN2. The method comprises exposing a GCN2 to an effective amount of an GCN2 modulator (inhibitor/activator) or related compound described herein, such as a compound of Formula I, to inhibit/activate GCN2 activity. In certain embodiments, the particular compound of Formula I, is the compound defined by one of the embodiments described above.

### Combination Therapy

Another aspect of the invention provides for combination therapy. GCN2 modulators (inhibitors/activators) and related compounds (e.g., a compound of Formula I) or their pharmaceutically acceptable salts may be used in combination with additional therapeutic agents to treat medical disorders, such as a cancer.

Exemplary therapeutic agents that may be used as part of a combination therapy in treating cancer, include, for example, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

Radiation therapy may also be used as part of a combination therapy.

An additional class of agents that may be used as part of a combination therapy in treating cancer is immune checkpoint inhibitors (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAB3, (v) B7-H3, (vi) B7-H4, and (vii) TIM3. The CTLA4 inhibitor Ipilumumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytoxic agents (e.g., tyrosine-kinase inhibitors).

Yet other agents that may be used as part of a combination therapy in treating cancer are agents which deplete amino acids or other nutrients, radiation, and agents that provoke the integrated stress response or that promote autophagy. Such agents may include aspariginase, argininase inhibitors of kinases such a b-Raf, and cytotoxic agents such as cis-platin.

Accordingly, another aspect of the invention provides a method of treating cancer in a patient, where the method comprises administering to the patient in need thereof (i) a therapeutically effective amount of a GCN2 modulator (activator/inhibitor) compound described herein and (ii) a second anti-cancer agent, in order to treat the cancer, where the second therapeutic agent may be one of the additional therapeutic agents described above (e.g., mitomycin, tretinoin, ribomustin, gemcitabine, an immune checkpoint inhibitor, or a monoclonal antibody agent that targets non-checkpoint targets) or one of the following:
an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor;
an agonist of OX40, CD137, CD40, GITR, CD27, HVEM, TNFRSF25, or ICOS;
a therapeutic antibody targeting one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, interleukin-1 beta, B4GALNT1, interleukin-6, and interleukin-6 receptor;
a cytokine selected from IL-12, IL-15, GM-CSF, and G-CSF;
a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine; or
a placental growth factor, an antibody-drug conjugate, an oncolytic virus, or an anti-cancer vaccine.

In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor.

In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40, CD137, CD40, GITR, CD27, HVEM, TNFRSF25, or ICOS. In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Moyl8, farletuzumab, 3F8, chl4.18, KW-2871, hu3S193, lgN311, bevacizumab, IM-2C6, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MEDI9447, daratumumab, belimumab, canakinumab, dinutuximab, siltuximab, and tocilizumab.

In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consisting of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine.

The doses and dosage regimen of the active ingredients used in the combination therapy may be determined by an attending clinician. In certain embodiments, the GCN2 modulator (inhibitor/activator) or related compound (e.g., a compound of any one of Formula I) and the additional therapeutic agent(s) are administered in doses commonly employed when such agents are used as monotherapy for treating the disorder. In other embodiments, the GCN2 modulator (inhibitor/activator) or related compound (e.g., a compound of any one of Formula I) and the additional therapeutic agent(s) are administered in doses lower than the doses commonly employed when such agents are used as monotherapy for treating the disorder. In certain embodiments, GCN2 modulator (inhibitor/activator) or related compound (e.g., a compound of any one of Formula I) and the additional therapeutic agent(s) are present in the same composition, which is suitable for oral administration.

In certain embodiments, the GCN2 modulator (inhibitor/activator) or related compound (e.g., a compound of any one of Formula I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the GCN2 modulator (inhibitor/activator) or related compound (e.g., a compound of any one of Formula I), a pharmaceutically acceptable carrier, vehicle or diluent, and optionally at least one additional therapeutic agent listed above.

### Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis), powders, granules, or as a solution or a suspension in an aqueous or non-aqueous liquid, or as an oil-in-water or water-in-oil liquid emulsion, or as an elixir or syrup, or as pastilles (using an inert base) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers and/or any of the following: (1) fillers or extenders; (2) binders; (3) humectants; (4) disintegrating agents; (5) solution retarding agents; (6) absorption accelerators, such as quaternary ammonium compounds and surfactants; (7) wetting agents; (8) absorbents; (9) lubricants; (10) coloring agents; and (11) controlled release agents. In the case of capsules, tablets and pills, the pharmaceutical compositions may also comprise buffering agents.

A tablet may be made by compression or molding, optionally with one or more accessory ingredients. Compressed tablets may be prepared using binder, lubricant, inert diluent, preservative, disintegrant, surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents.

Formulations of the pharmaceutical compositions of the invention for rectal or vaginal administration may be presented as a suppository, which may be prepared by mixing one or more compounds of the invention with one or more suitable nonirritating excipients or carriers which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Proper fluidity can be maintained, for example, by the use of coating materials, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents. It may also be desirable to include isotonic agents into the compositions.

In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers. Depending on the ratio of drug to polymer, and the nature of the particular polymer employed, the rate of drug release can be controlled. Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising an (aza)indazolyl-aryl sulfonamide or related compound described herein in a therapeutically effective amount for the treatment of a medical disorder described herein.

### EXAMPLES

The representative examples that follow are intended to help illustrate the invention, and are not intended to, nor should they be construed to, limit the scope of the invention.

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimal reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include recrystallization, filtration, flash chromatography, trituration, high pressure liquid chromatography (HPLC), or supercritical fluid chromatography (SFC). Note that flash chromatography may either be performed manually or via an automated system. The compounds provided herein may be characterized by known standard procedures, such as nuclear magnetic resonance spectroscopy (NMR) or liquid chromatography mass spectrometry (LCMS). NMR chemical shifts are reported in part per million (ppm) and are generated using methods well known to those of skill in the art.

### List of Abbreviations

Ac acetyl
ACN acetonitrile
AcOH acetic acid
AIBN azobisisobutyronitrile
BAST bis(2-methoxyethyl)aminosulfur trifluoride
Boc tert-butyloxycarbonyl
BINAP 2,2′-bis(diphenylphosphino)-1,1′-binaphthyl
BINOL 1,1′-bi-2-naphthol
BPO benzoyl peroxide
COD 1,5-cyclooctadiene
Cy cyclohexyl
CDI carbonyldiimidazole
DAST diethylaminosulfur trifluoride
dba dibenzylideneacetone
DBU 1,8-diazabicyclo[5.4.0]undec-7-ene
DCE dichloroethane
DCM dichloromethane
DDQ 2,3-dichloro-5,6-dicyano-1,4-benzoquinone
DEAD diethyl azodicarboxylate
DHP dihydropyran
DIEA diisopropylethylamine
DMA dimethylacetamide
DMAP 4-dimethylaminopyridine
DMF dimethylformamide
DMSO dimethyl sulfoxide
dppf 1,1′-bis(diphenylphosphino)ferrocene
dtbbpy 4,4′-di-tert-butyl-2,2′-dipyridyl
dtbpf 1,1′-bis(di-tert-butylphosphino)ferrocene
EA ethyl acetate
EDTA Ethylenediaminetetraacetic acid
EtOAc ethyl acetate
FA formic acid
HATU 1-[Bis(dimethylamino)methylene]-1H-1,2,3-triazolo[4,5-b]pyridinium-3-oxide hexafluorophosphate
HMDS bis(trimethylsilyl)amide
HPLC high performance liquid chromatography
IBX 2-iodoxybenzoic acid
IPA isopropyl alcohol
LAH lithium aluminum hydride
LCMS liquid chromatography-mass spectrometry
LDA lithium diisopropylamide
MeCN acetonitrile
MTBE methyl tert-butyl ether
NBS N-bromosuccinimide
NCS N-chlorosuccinimide
NIS N-iodosuccinimide
NMI N-methylimidazole
NMP N-methylpyrrolidinone
NMR nuclear magnetic resonance spectroscopy
PE petroleum ether
Pin pinacolato
PMB p-methoxybenzyl
Py pyridine
rt room temperature
RT retention time
SEM trimethylsilylethoxymethyl
SFC super-critical fluid chromatography
TBAB tetrabutylammonium bromide
TBAF tetrabutylammonium fluoride
TBDPS tert-butyldiphenylsilyl
TBS tert-butyldimethylsilyl
TCFH Chloro-N,N,N′,N′-tetramethylformamidinium hexafluorophosphate
TEA triethylamine
Tf triflate/trifluoromethanesulfonate
TFA trifluoroacetic acid
TFAA trifluoroacetic acid anhydride
TMAD N,N,N′,N′-Tetramethylazodicarboxamide
TMS trimethylsilyl
Ts tosyl

### General Schemes

In some embodiments, compounds of the present disclosure may be manufactured using a process comprising one or more of Schemes 1-14 as set out below. Reaction steps represented by dashed arrows are to be understood to be optional. Unless otherwise specified, the variables of the Schemes are as defined herein. Reaction conditions should be understood to be exemplary and non-limiting, and may occur in the presence of an appropriate solvent.

In some embodiments, compounds of the present disclosure may be synthesized using a process comprising Scheme 1, wherein Z is halide, R is alkyl, R^{a} is alkyl, and R^{b} is substituted or unsubstituted alkyl.

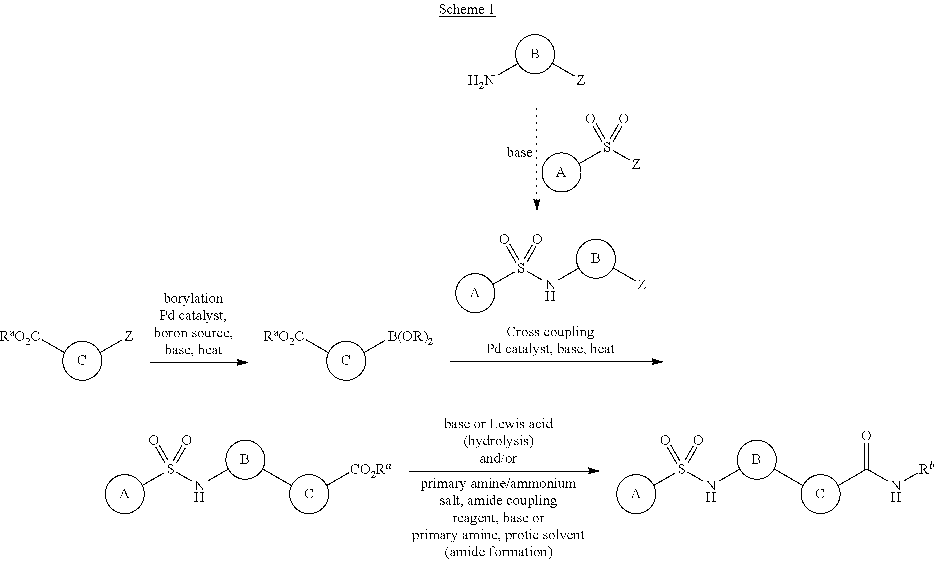

In some embodiments, compounds of the present disclosure may be synthesized using a process comprising Scheme 2, wherein Z is halide, R is alkyl, R^{a} is alkyl, and R^{b} is substituted or unsubstituted alkyl.

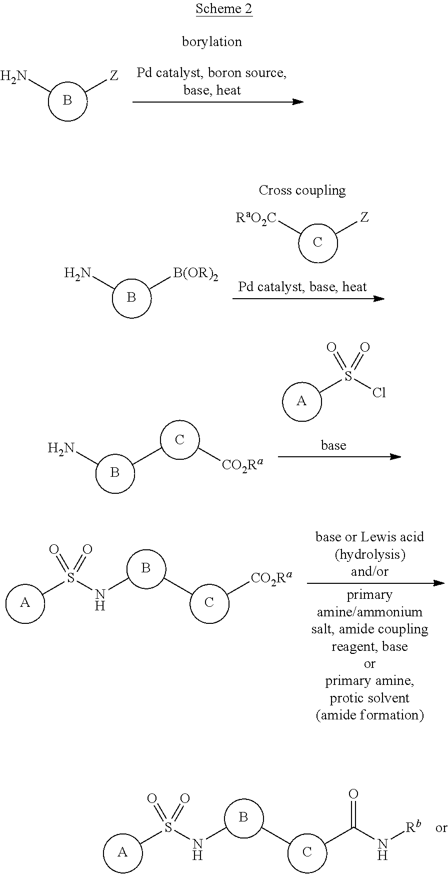

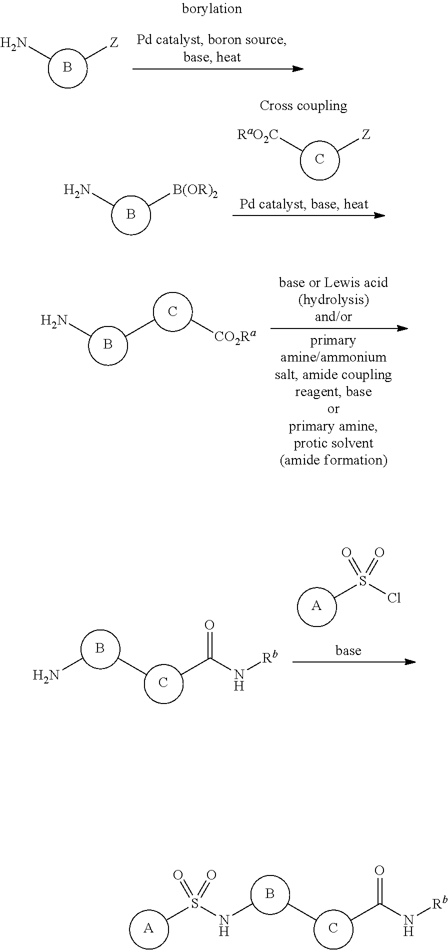

In some embodiments, compounds of the present disclosure may be synthesized using a process comprising Scheme 3, wherein Z is halide, R is alkyl, R^{a} is methyl, and R^{b} is substituted or unsubstituted alkyl.

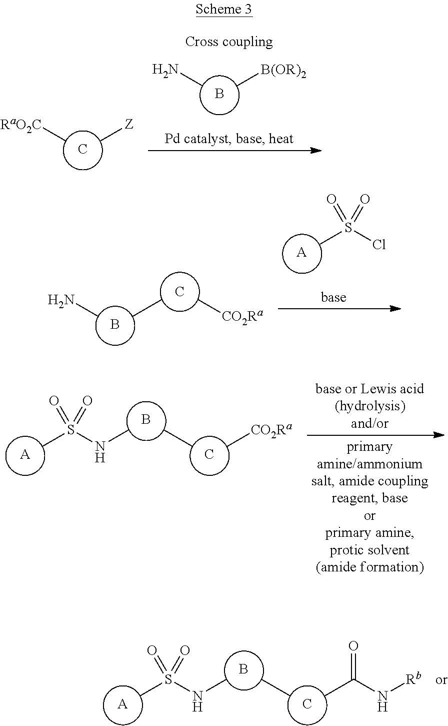

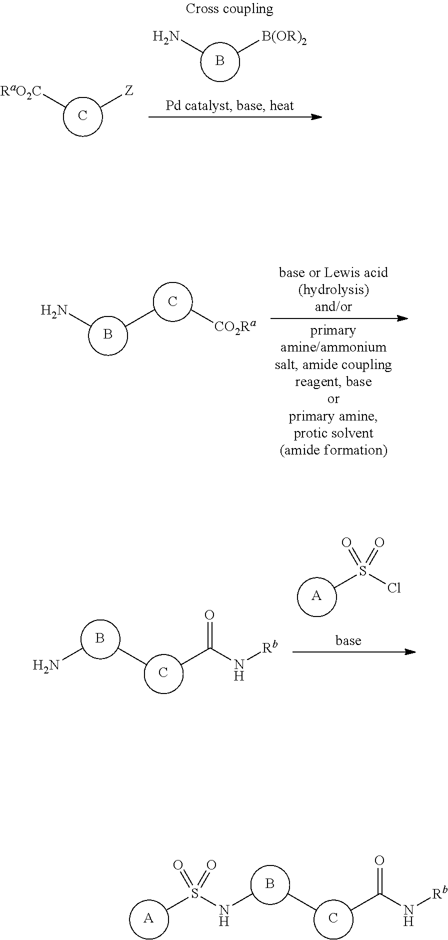

In some embodiments, compounds of the present disclosure may be synthesized using a process comprising Scheme 4, wherein Z is halide, Y is amide or ester, and R is alkyl.

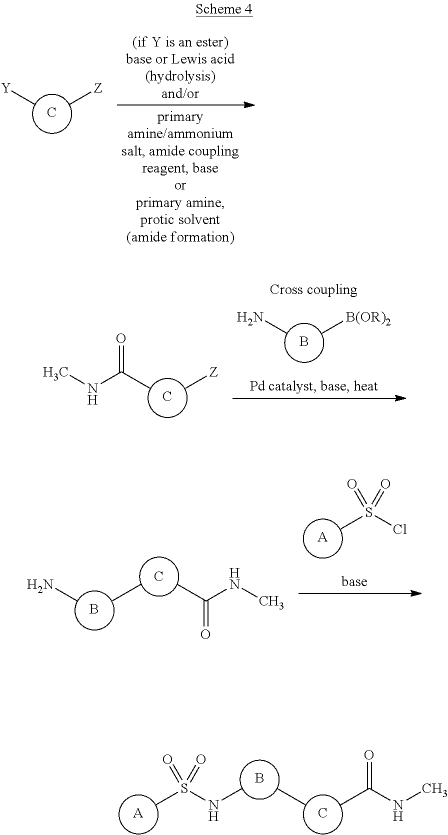

In some embodiments, compounds of the present disclosure may be synthesized using a process comprising Scheme 5, wherein Z is halide, Y is amide or ester, and R is alkyl.

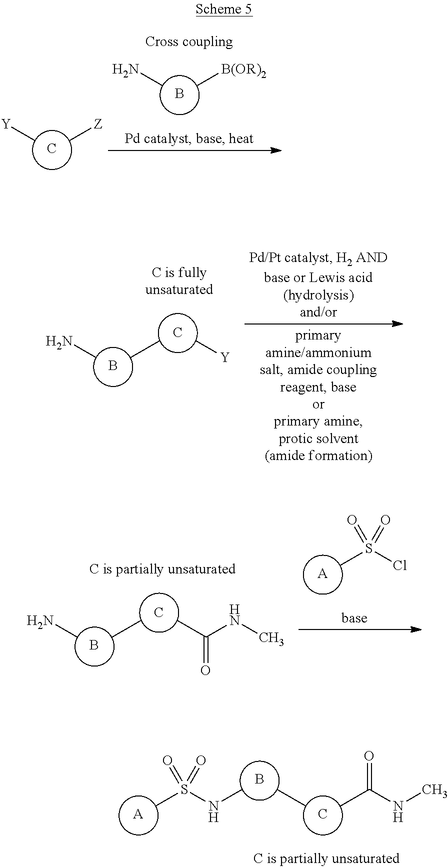

In some embodiments, compounds of the present disclosure may be synthesized using a process comprising Scheme 6, wherein Z is halide, R is alkyl, and R^{a} is alkyl.

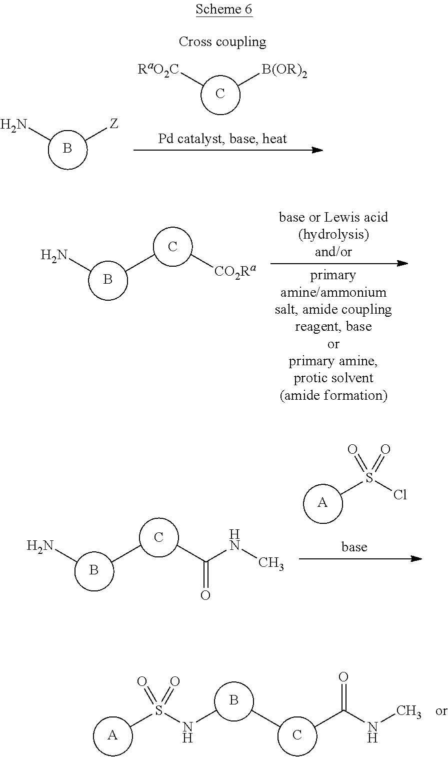

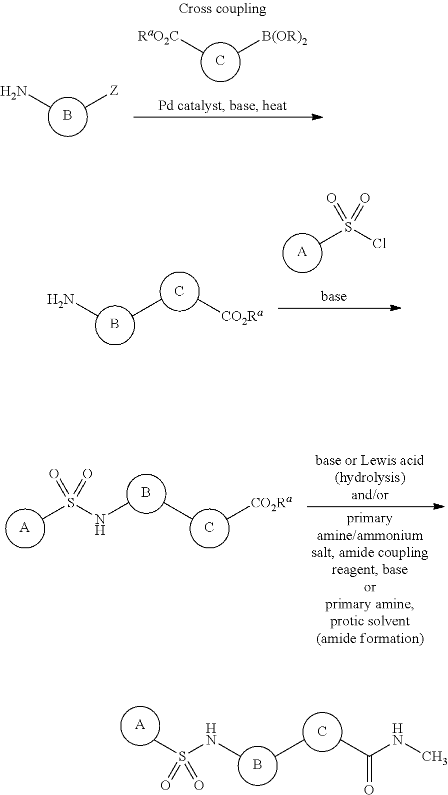

In some embodiments, compounds of the present disclosure may be synthesized using a process comprising Scheme 7, wherein Z is halide, R is alkyl, and Hy is heterocycle or heteroaryl.

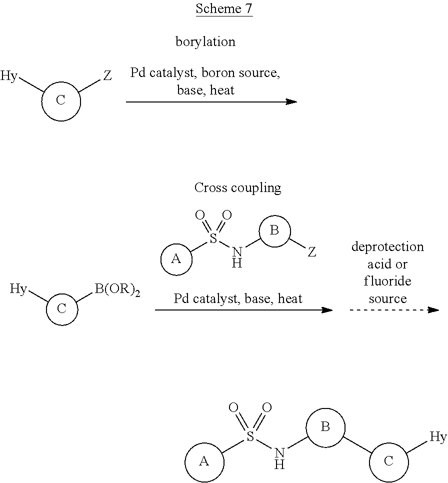

In some embodiments, compounds of the present disclosure may be synthesized using a process comprising Scheme 8, wherein Z is halide, R is alkyl, and Hy is heterocycle or heteroaryl.

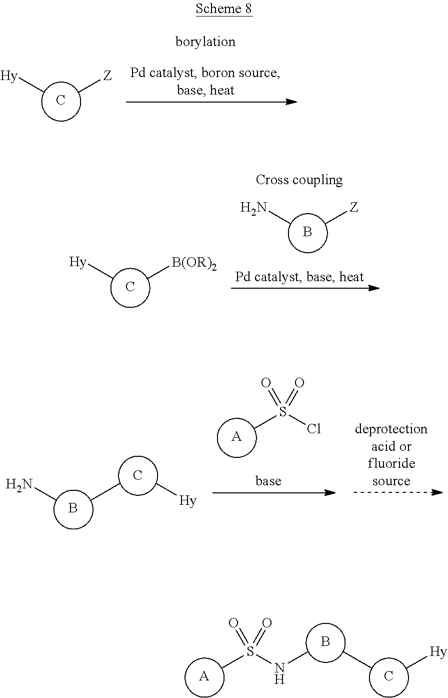

In some embodiments, compounds of the present disclosure may be synthesized using a process comprising Scheme 9, wherein Z is halide, R is alkyl, and Hy is heterocycle or heteroaryl.

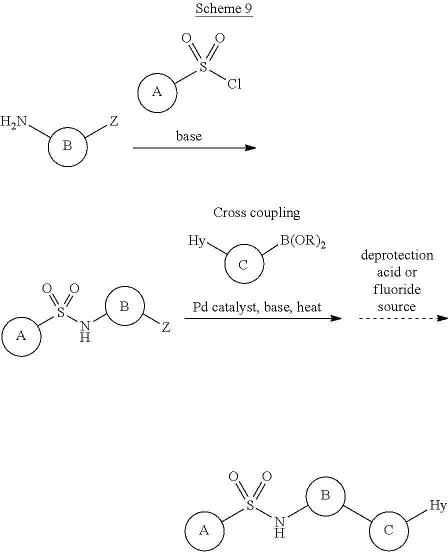

In some embodiments, compounds of the present disclosure may be synthesized using a process comprising Scheme 10, wherein Z is halide, R is alkyl, FG is a functional group that may be readily converted to a heterocycle or heteroaryl, and Hy is heterocycle or heteroaryl. In some embodiments, FG is cyano and may be reacted with, for example, an amino aldehyde acetal with base and/or acid, or an amidine reagent with base, to form a heterocycle or heteroaryl. In some embodiments, FG is halogen and may be reacted with a heterocyclyl or heteroaryl organolithium reagent and metal catalyst.

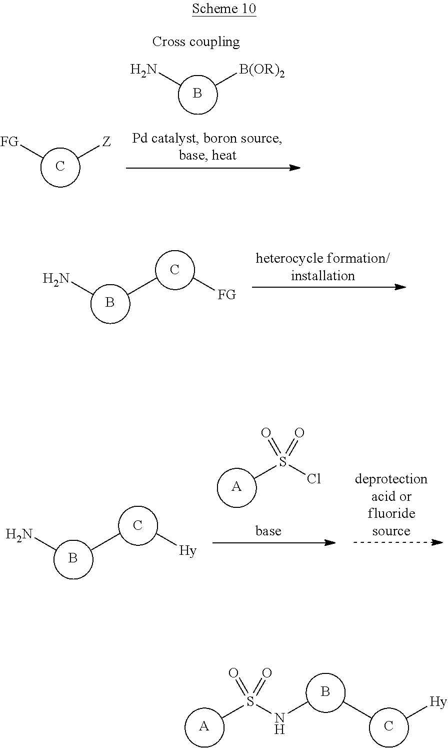

In some embodiments, compounds of the present disclosure may be synthesized using a process comprising Scheme 11, wherein Z is halide, R is alkyl, and Hy is heterocycle or heteroaryl.

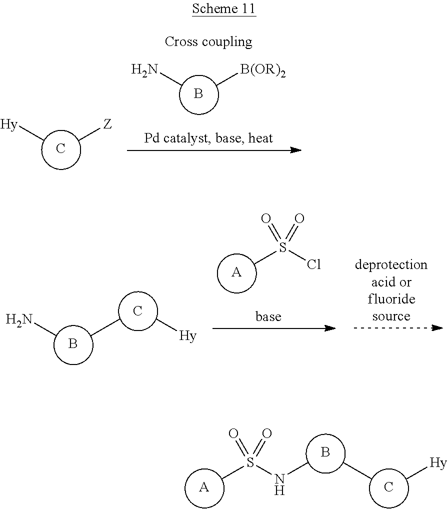

In some embodiments, compounds of the present disclosure may be synthesized using a process comprising Scheme 12, wherein FG is a functional group that may be readily converted to a heterocycle or heteroaryl, and Hy is heterocycle or heteroaryl. In some embodiments, FG is cyano and may be reacted with, for example, an amino aldehyde acetal with base and/or acid, or an amidine reagent with base, to form a heterocycle or heteroaryl.

In some embodiments, FG is halogen and may be reacted with a heterocyclyl or heteroaryl organolithium and metal catalyst.

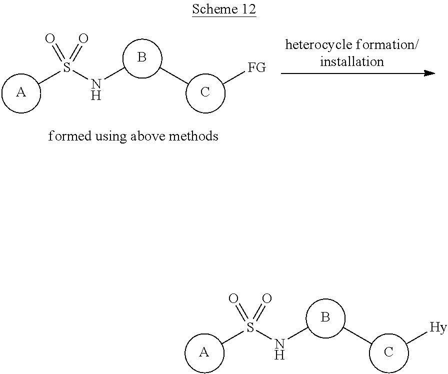

In some embodiments, compounds of the present disclosure may be synthesized using a process comprising Scheme 13, wherein Hy is heterocycle or heteroaryl.

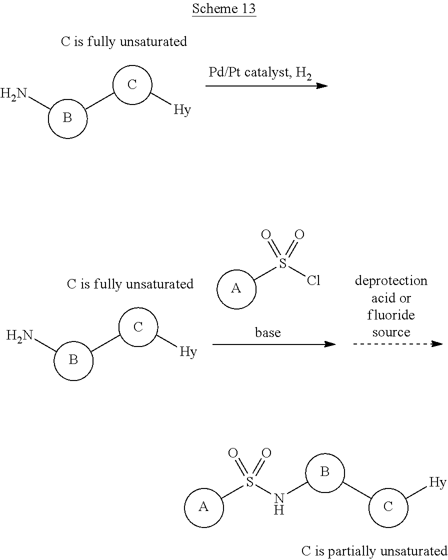

In some embodiments, compounds of the present disclosure may be synthesized using a process comprising Scheme 14, wherein W is —NCH₃ or —OCH₃, and R^{c} is H or methyl.

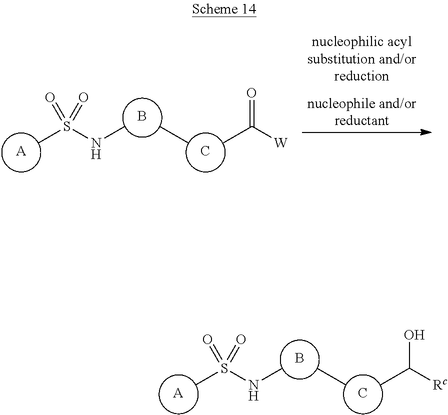

### Example 1: Synthesis of 5-fluoro-2-methoxypyridine-3-sulfonyl chloride (Intermediate 1)

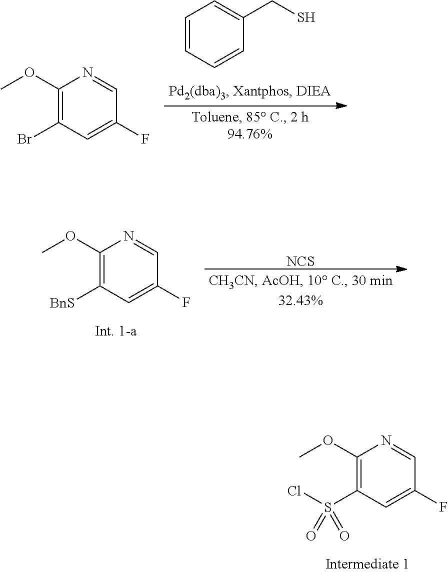

### Synthesis of Int. 1-a: 3-(benzylsulfanyl)-5-fluoro-2-methoxypyridine

Into a 2000 mL 3-necked round-bottom flask purged and maintained with an inert atmosphere of nitrogen, was placed 3-bromo-5-fluoro-2-methoxypyridine (150 g, 728 mmol, 1 equiv.), benzyl mercaptan (109 g, 878 mmol, 1.2 equiv), Pd₂(dba)₃ (41.9 g, 36 mmol, 0.05 equiv.), Xantphos (30 g, 52 mmol, 0.07 equiv), DIEA (189 g, 1.46 mol) and toluene (1.2 L). The resulting solution was stirred for 2 h at 85° C. in an oil bath, then concentrated under vacuum. The residue was applied to a silica gel column, eluting with ethyl acetate/petroleum ether (1:10) to give 3-(benzylsulfanyl)-5-fluoro-2-methoxypyridine (172 g, 95% yield) as a colorless oil.

### Synthesis of Int. 1: 5-fluoro-2-methoxypyridine-3-sulfonyl chloride

Into a 2000 mL 3-necked round-bottom flask purged and maintained with an inert atmosphere of nitrogen, was placed 3-(benzylsulfanyl)-5-fluoro-2-methoxypyridine (172 g, 690 mmol, 1 equiv.) and CH₃CN (1000 mL). This was followed by the addition of HCl (57 mL) at 10° C. To this was added NCS (368.5 g, 2760 mmol, 4 equiv) in portions at 10° C. The resulting solution was stirred for 30 min at 10˜20° C. in a water/ice bath. The resulting solution was diluted with 2000 mL of H₂O and extracted with 2×1.5 L of dichloromethane. The combined organics were washed with 2000 ml of brine and dried over anhydrous sodium sulfate, before being concentrated. The residue was applied to a silica gel column which was eluted with PE. 5-Fluoro-2-methoxypyridine-3-sulfonyl chloride (50.5 g, 32% yield) was isolated as a white solid.

¹H-NMR: (300 MHz, Chloroform-d, ppm): δ 8.366 (d, J=3.0 Hz, 1H), 8.043-8.011 (m, 1H), 4.178 (s, 3H).

### Example 2: Synthesis of 5-chloro-2-methoxypyridine-3-sulfonyl chloride (Intermediate 2)

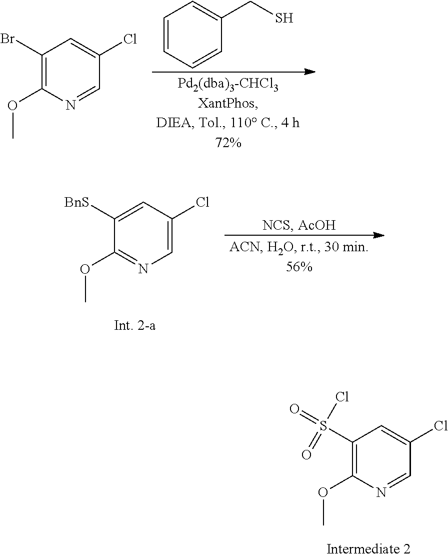

### Synthesis of Int. 2-a: 3-(benzylsulfanyl)-5-chloro-2-methoxypyridine

Into a 3000 mL round-bottom flask purged and maintained with an inert atmosphere of nitrogen, was placed 3-bromo-5-chloro-2-methoxypyridine (150 g, 678 mmol, 1.0 equiv), toluene (1500 mL), phenylmethanethiol (92.6 g, 746 mmol, 1.1 equiv), DIEA (175.4 g, 1357 mmol, 2.0 equiv), XantPhos (3.9 g, 6 mmol, 0.01 equiv) and Pd₂(dba)₃-CHCl₃ (5.3 g, 5 mmol 0.0075 equiv). The resulting solution was stirred for 4 h at 110° C. in an oil bath. The solids were removed by filtration and the filtrate concentrated. The residue was applied onto a silica gel column, eluting with PE:EA=20: 1. Concentration of the appropriate fractions gave a solid which was slurried with PE (3 V). The solid was removed by filtration and dried to give 3-(benzylsulfanyl)-5-chloro-2-methoxypyridine (130 g, 72% yield) as a yellow solid.

### Synthesis of Int. 2: 5-chloro-2-methoxypyridine-3-sulfonyl chloride

Into a 4000 mL round-bottom flask, was placed 3-(benzylsulfanyl)-5-chloro-2-methoxypyridine (130 g, 490 mmol, 1 equiv), MeCN (2600 mL, 20 V), H₂O (130 mL, 1 V), acetic acid (294 g, 491 mmol, 10 equiv) and NCS (196 g, 1471 mmol, 3.0 equiv). The resulting solution was stirred for 30 min at 25° C. The reaction was quenched by the addition 1000 mL of water, and extracted with 2×1000 mL of ethyl acetate. The combined organics were washed with H₂O and concentrated. The resulting solution was diluted with 500 mL of diethyl ether and the solids removed by filtration. The filtrate was concentrated, and the residue purified by silica gel column chromatography, eluting with PE/THF (100:1). The concentrated product was slurried with 300 mL hexane and kept at 0° C. for 1 h. The solids were removed by filtration and dried to give 5-chloro-2-methoxypyridine-3-sulfonyl chloride (66.5 g, 56% yield) as a white solid.

¹H-NMR: (300 MHz, CDCl₃, ppm): δ 8.45 (d, J=2.5 Hz, 1H), 8.23 (d, J=2.6 Hz, 1H), 4.21 (s, 3H).

### Example 3: Synthesis of 5-cyano-2-methoxypyridine-3-sulfonyl chloride (Intermediate

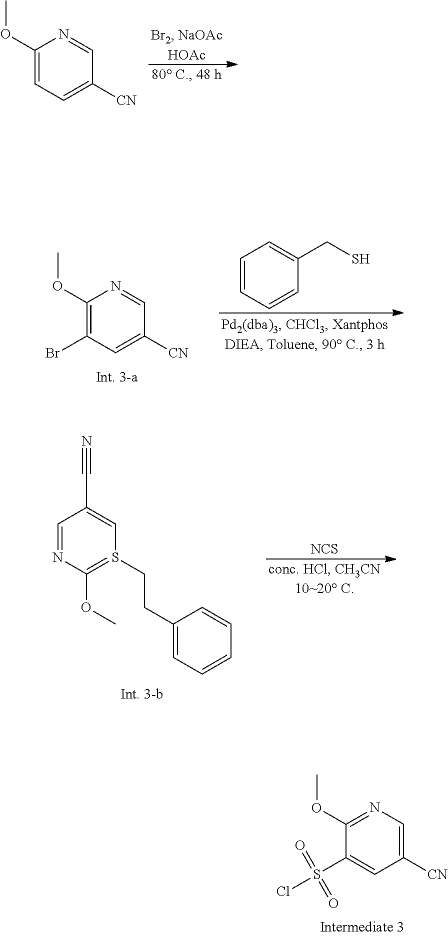

### Synthesis of Int. 3-a: 5-bromo-6-methoxypyridine-3-carbonitrile

Into a 2 L round-bottom flask were added 6-methoxypyridine-3-carbonitrile (100 g, 746 mmol, 1 equiv), HOAc (1000 mL), NaOAc (61 g, 746 mmol, 1 equiv) and Br₂ (235 g, 1490 mmol, 2 equiv) at room temperature. The resulting mixture was stirred for 48 h at 80° C. The mixture was allowed to cool to room temperature and was diluted with ice water (3 L). The precipitated solids were collected by filtration and suspended in 1 L of PE:EA=5:1 which was stirred for 1 h at room temperature. The precipitated solids were collected by filtration and dried to give 5-bromo-6-methoxypyridine-3-carbonitrile (70 g, 44% yield) as a light yellow solid.

### Synthesis of Int. 3-b: 5-(benzylsulfanyl)-6-methoxypyridine-3-carbonitrile

Into a 3 L 3-necked round-bottom flask, were added 5-bromo-6-methoxypyridine-3-carbonitrile (70 g, 330 mmol, 1 equiv), toluene (1400 mL) and benzyl mercaptan (43 g, 347 mmol, 1.05 equiv) at room temperature. To this was added Pd₂(dba)₃·CHCl₃ (17 g, 16.5 mmol, 0.05 equiv), Xantphos (19 g, 33 mmol, 0.1 equiv) and DIEA (128 g, 990 mmol, 3 equiv) under nitrogen atmosphere. The resulting mixture was stirred for 3 h at 90° C. under nitrogen atmosphere, then was cooled and filtered. The filtrate was concentrated under reduced pressure, and the residue purified by silica gel column chromatography, eluting with PE/THF (10:1). 5-(Benzylsulfanyl)-6-methoxypyridine-3-carbonitrile (65 g, 77% yield) was obtained as a light brown solid.

### Synthesis of Int. 3: 5-cyano-2-methoxypyridine-3-sulfonyl chloride

Into a 2 L 3-necked round-bottom flask were added 5-(benzylsulfanyl)-6-methoxypyridine-3-carbonitrile (65 g, 2540 mmol, 1 equiv), MeCN (520 g), H₂O (260 g) and HCl (21 mL, 254 mmol, 1 equiv) at room temperature. To this was added NCS (101 g, 762 mmol, 3 equiv) in portions at 0° C. The resulting mixture was stirred for 0.5 h at room temperature, then was cooled to 0° C. and stirred for 1 h. The precipitated solids were collected by filtration and dried to afford 5-cyano-2-methoxypyridine-3-sulfonyl chloride (25 g, 42% yield) as a white solid.

H-NMR: (300 MHz, CDCl3, ppm): δ 8.78 (d, J=2.2 Hz, 1H), 8.51 (d, J=2.2 Hz, 1H), 4.31 (s, 3H).

### Example 4: Synthesis of 5-fluoro-2-methylpyridine-3-sulfonyl chloride (Intermediate 4)

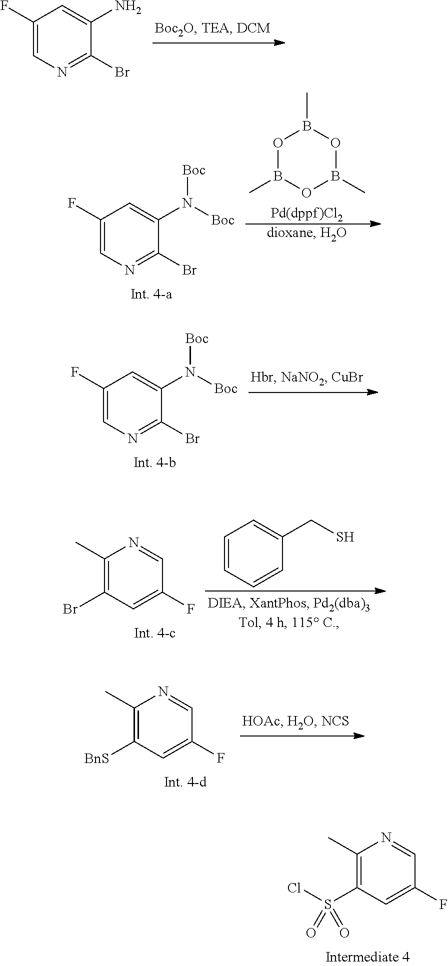

### Synthesis of Int. 4-a: tert-butyl N-(2-bromo-5-fluoropyridin-3-yl)-N-(tert-butoxycarbonyl)carbamate

Into a 500 mL 3-necked round-bottom flask was placed 2-bromo-5-fluoropyridin-3-amine (20 g, 1 equiv), DCM (220 mL) and TEA (44 mL, 3 equiv). This was followed by the addition of Boc₂O (57 g, 2.5 equiv) in several batches at 26° C. The resulting solution was stirred for 14 h at 26° C. The reaction mixture was concentrated, and the residue was applied to a silica gel column, eluting with ethyl acetate/petroleum ether (1:5). This resulted in tert-butyl N-(2-bromo-5-fluoropyridin-3-yl)-N-(tert-butoxycarbonyl)carbamate (33 g) as a white solid.

### Synthesis of Int. 4-b: tert-butyl N-(tert-butoxycarbonyl)-N-(5-fluoro-2-methylpyridin-3-yl)carbamate

Into a 500 mL 3-necked round-bottom flask, purged and maintained with an inert atmosphere of nitrogen, was placed tert-butyl N-(2-bromo-5-fluoropyridin-3-yl)-N-(tert-butoxycarbonyl)carbamate (33 g, 84 mmol, 1 equiv), 1,4-dioxane (150 mL), trimethyl-1,3,5,2,4,6-trioxatriborinane (21.2 g, 169 mmol, 2 equiv), K₂CO₃ (35 g, 253 mmol, 3 equiv) and Pd(dppf)Cl₂ (3.09 g, 4.2 mmol, 0.05 equiv). The resulting solution was stirred overnight at 110° C. under N₂ atmosphere. The reaction mixture was cooled to room temperature and filtered. The filtrate was diluted with 200 mL of H₂O and extracted with 3×100 mL of ethyl acetate. The combined organics were concentrated and the residue applied to a silica gel column, eluting with ethyl acetate/petroleum ether (1:10-1:5). Tert-butyl N-(tert-butoxycarbonyl)-N-(5-fluoro-2-methylpyridin-3-yl)carbamate (16 g, 58% yield) was isolated as a yellow solid.

### Synthesis of Int. 4-c: 3-bromo-5-fluoro-2-methylpyridine

Into a 2 L 3-necked round-bottom flask, was placed tert-butyl N-(tert-butoxycarbonyl)-N-(5-fluoro-2-methylpyridin-3-yl)carbamate (50 g, 153 mmol, 1 equiv) and HBr (1 L, 48%). This was followed by the addition of a solution of NaNO₂ (11.6 g, 169 mmol, 1.1 equiv) in H₂O (100 mL) dropwise with stirring at 0-5° C. The resulting solution was stirred for 30 min in an ice bath. To this was added CuBr (24.2 g, 169 mmol, 1.1 equiv) at 0° C. The resulting solution was stirred for 1 h at room temperature, then quenched by the addition of 1 L water/ice. The pH of the solution was adjusted to 8 with Na₂CO₃ and the resulting mixture was extracted with 3×200 mL of ethyl acetate. The combined organics were dried over anhydrous sodium sulfate and concentrated. The residue was applied to a silica gel column, eluting with ethyl acetate/petroleum ether (1:50). 3-Bromo-5-fluoro-2-methylpyridine (13 g, 45% yield) was isolated as a white solid.

### Synthesis of Int. 4-d: 3-(benzylsulfanyl)-5-fluoro-2-methylpyridine

Into a 250 mL 3-necked round-bottom flask, purged and maintained with an inert atmosphere of nitrogen, was placed 3-bromo-5-fluoro-2-methylpyridine (13 g, 68 mmol, 1 equiv), toluene (130 mL), benzyl mercaptan (12.8 g, 103 mmol, 1.5 equiv), DIEA (17.7 g, 137 mmol, 2 equiv), XantPhos (3.96 g, 6.8 mmol, 0.1 equiv) and Pd₂(dba)₃ (3.13 g, 3.4 mmol, 0.05 equiv). The resulting solution was stirred for 4 h at 115° C., then cooled and concentrated. The residue was applied to a silica gel column, eluting with ethyl acetate/petroleum ether (1:20) to give 3-(benzylsulfanyl)-5-fluoro-2-methylpyridine (11 g, 69% yield) as a yellow solid.

### Synthesis of Int. 4: 5-fluoro-2-methylpyridine-3-sulfonyl chloride

Into a 2000 mL 3-necked round-bottom flask, was placed 3-(benzylsulfanyl)-5-fluoro-2-methylpyridine (45 g, 193 mmol, 1 equiv), HOAc (700 mL) and H₂O (200 mL). This was followed by the addition of NCS (103 g, 772 mmol, 4 equiv), the temperature being maintained under 20° C. The resulting solution was stirred for 2 h at room temperature. The reaction was quenched by the addition of 700 mL of water, and the resulting solution was extracted with 3×300 mL of dichloromethane. The combined organics were concentrated, and the residue applied to a silica gel column, eluting with ethyl acetate/petroleum ether (1:10). 5-Fluoro-2-methylpyridine-3-sulfonyl chloride (25.4 g, 63% yield) was obtained as a yellow oil.

LCMS: (ES, m/z): [M+1]⁺=210

¹H-NMR: (300 MHz, CDCl₃, ppm): δ 8.72-8.71 (d, J=3.0 Hz, 1H), 8.11-8.08 (dd, J=3.0 Hz, 1H), 3.01 (s, 3H).

### Example 5: Synthesis of 5-chloro-N-[3,5-difluoro-4-[1-(1H-imidazol-2-yl)imidazo[1,5-a]pyridin-6-yl]pyridin-2-yl]-2-methoxypyridine-3-sulfonamide (Intermediate 5)

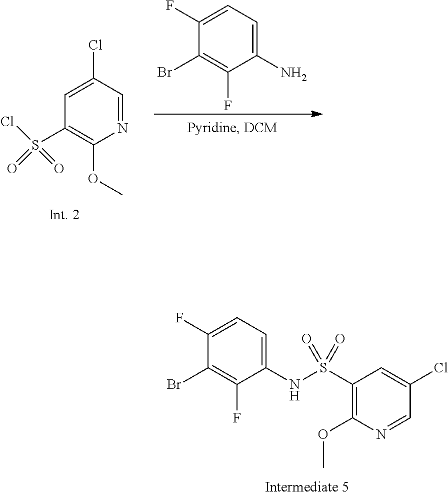 [SMILES: COc1ncc(Cl)cc1S(=O)(=O)Cl ; Nc1ccc(F)c(Br)c1F ; COc1ncc(Cl)cc1S(=O)(=O)Nc1ccc(F)c(Br)c1F]

### Synthesis of Intermediate 5: N-(3-bromo-2,4-difluorophenyl)-5-chloro-2-methoxypyridine-3-sulfonamide

To a solution of 3-bromo-2,4-difluoroaniline (5 g, 24 mmol, 1 eq) in DCM (100 mL) were added 5-chloro-2-methoxypyridine-3-sulfonyl chloride (8.73 g, 36 mmol, 1.5 eq) and pyridine (5.7 g, 72 mmol, 3 eq). The resulting solution was stirred for 1 hour at room temperature. The reaction was concentrated and purified by column chromatography over silica gel (eluent: PE:EA=8:1) to afford N-(3-bromo-2,4-difluorophenyl)-5-chloro-2-methoxypyridine-3-sulfonamide (7 g, 70% yield) as a white solid.

LCMS (ES, m/z): [M+H]⁺: 412

¹H NMR (300 MHz, Chloroform-d) δ 8.30 (d, J=2.6 Hz, 1H), 8.05 (d, J=2.6 Hz, 1H), 7.56 (td, J=8.9, 5.4 Hz, 1H), 7.28 (d, J=3.4 Hz, 1H), 6.96 (ddd, J=9.4, 7.6, 2.1 Hz, 1H), 4.16 (s, 3H).

### Example 6: Synthesis of ethyl 6-bromoimidazo[1,5-a]pyridine-1-carboxylate (Intermediate 6)

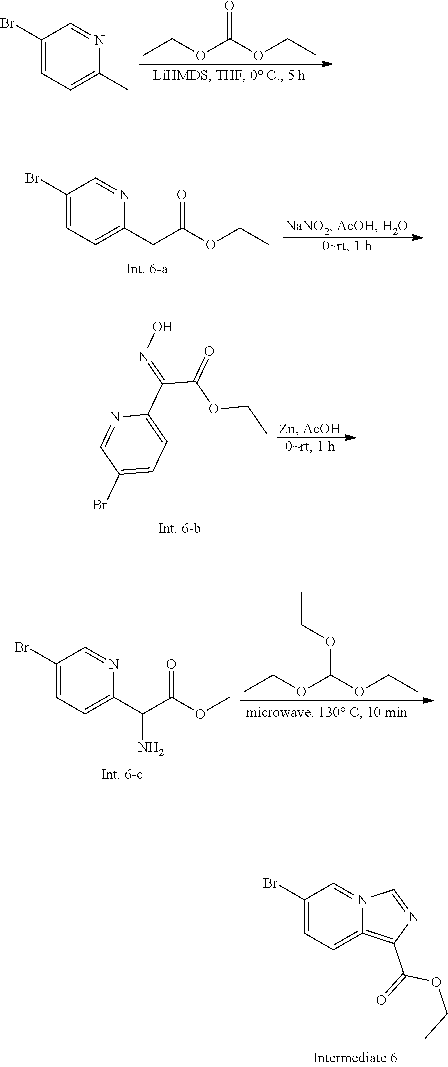

### Synthesis of Int. 6-a ethyl 2-(5-bromopyridin-2-yl)acetate

Into a 250 mL 3-necked round-bottom flask, purged and maintained with an inert atmosphere of nitrogen, was placed a solution of 5-bromo-2-methylpyridine (3 g, 17 mmol, 1 equiv) in THF (100 mL). This was followed by the addition of LiHMDS in THF (34 mL, 34 mmol, 2 equiv) dropwise with stirring at 0° C. over 30 min. To this was added diethyl carbonate (3.1 g, 26 mmol, 1.5 equiv) at 0° C. and the resulting solution was stirred for 5 h at room temperature. The reaction was quenched by the addition of 30 mL of H₂O, and the resulting mixture was concentrated under vacuum. The residue was diluted with 100 mL of EA, and the organics washed with H₂O (2×100 mL) and brine (100 mL). After drying over anhydrous sodium sulfate, the solution was concentrated under vacuum to give ethyl 2-(5-bromopyridin-2-yl)acetate (3.0 g crude) as a yellow oil.

LCMS (ES, m/z): [M+H]⁺: 244

### Synthesis of Int. 6-b: ethyl 2-(5-bromopyridin-2-yl)-2-(N-hydroxyimino)acetate

Into a 25 mL 3-necked round-bottom flask, purged and maintained with an inert atmosphere of nitrogen, was placed a solution of ethyl 2-(5-bromopyridin-2-yl)acetate (2.9 g, 12 mmol, 1 equiv) in AcOH (5 mL). This was followed by the addition of a solution of NaNO₂ (823 mg, 12 mmol, 1 equiv) in H₂O (2 mL) dropwise with stirring at 0° C. The resulting solution was stirred for 1 hr at room temperature, then diluted with 20 mL of H₂O. The mixture was extracted with 2×20 mL of ethyl acetate and the combined organics washed with 30 ml of brine, then dried over anhydrous sodium sulfate. Concentration gave ethyl 2-(5-bromopyridin-2-yl)-2-(N-hydroxyimino)acetate (2.3 g) as a yellow oil which was used in next step directly without further purification.

LCMS (ES, m/z): [M+H]⁺: 273

### Synthesis of Int. 6-c: ethyl 2-amino-2-(5-bromopyridin-2-yl)acetate

Into a 100 mL 3-necked round-bottom flask, was placed a solution of ethyl 2-(5-bromopyridin-2-yl)-2-(N-hydroxyimino)acetate (2.3 g, 8.3 mmol, 1 equiv) in AcOH (20 mL). This was followed by the addition of Zn (1.6 g, 25 mmol, 3 equiv) in portions at 0° C. over 10 min. The resulting solution was stirred for 60 min at room temperature. The solids were removed by filtration and the filtrate concentrated under vacuum to give crude ethyl 2-amino-2-(5-bromopyridin-2-yl)acetate (1.8 g) as a yellow oil which was used in next step directly without further purification.

LCMS (ES, m/z): [M+H]⁺: 259

### Synthesis of Intermediate 6: ethyl 6-bromoimidazo[1,5-a]pyridine-1-carboxylate

Into a 5 mL microwave tube, was placed ethyl 2-amino-2-(5-bromopyridin-2-yl)acetate (1.8 g, 7 mmol, 1 equiv) and triethyl orthoformate (3 mL). The resulting solution was stirred for 10 min at 130° C., then was cooled and concentrated. The residue was applied to a silica gel column, eluting with THF/PE (1:1). Ethyl 6-bromoimidazo[1,5-a]pyridine-1-carboxylate (530 mg) was isolated as a purple solid.

LCMS (ES, m/z): [M+H]⁺: 269

### Example 7: Synthesis of 6-bromo-1-iodoimidazo[1,5-a]pyridine (Intermediate 7)

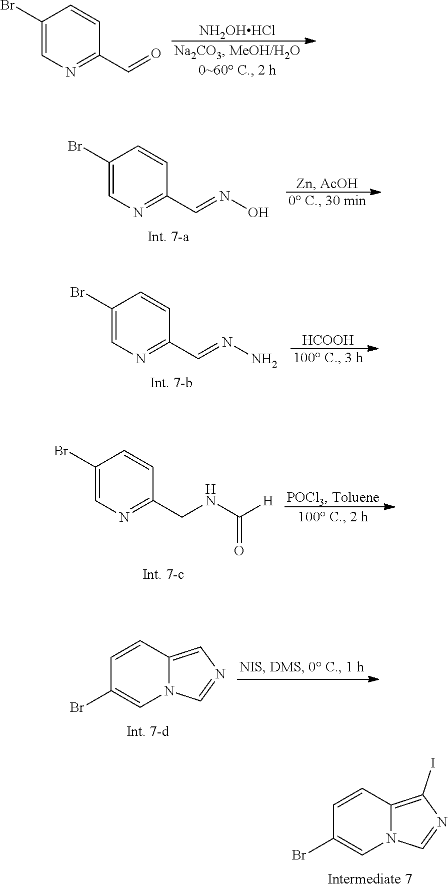 [SMILES: O=Cc1ccc(Br)cn1 ; O/N=C/c1ccc(Br)cn1 ; N/N=C/c1ccc(Br)cn1 ; O=CNCc1ccc(Br)cn1 ; Brc1ccc2cncn2c1 ; Brc1ccc2c(I)ncn2c1]

### Synthesis of Int. 7-a (E)-N-[(5-bromopyridin-2-yl)methylidene]hydroxylamine

Into a 500 mL 3-necked round-bottom flask was placed a solution of 5-bromopyridine-2-carbaldehyde (20 g, 0.11 mol, 1 equiv) in MeOH (150 mL), followed by a solution of Na₂CO₃ (23 g, 0.2 mol, 2 equiv) in H₂O (100 mL). Hydroxylamine hydrochloride (9.7 g, 0.14 mol, 1.3 equiv) was then added at 0° C. The resulting solution was stirred for 2 hr at 60° C. in an oil bath. The reaction was quenched by the addition of 500 mL of water/ice, and the solids were collected by filtration. Air drying yielded (E)-N-[(5-bromopyridin-2-yl)methylidene]hydroxylamine (21 g, 87% yield) as a white solid.

LCMS (ES, m/z): [M+H]⁺: 201

### Synthesis of Int. 7-b: 1-(5-bromopyridin-2-yl)methanamine

Into a 500 mL 3-necked round-bottom flask was placed (E)-N-[(5-bromopyridin-2-yl)methylidene]hydroxylamine (21 g, 0.1 mol, 1 equiv) and AcOH (200 mL). Zn (20.5 g, 0.3 mol, 3 equiv) was then added in portions at 0-10° C. The resulting solution was stirred for 30 min at 0-10° C. in an ice/salt bath. The solids were filtered out and the filtrate concentrated under vacuum. The resulting mixture was diluted with 200 mL of H₂O, and the pH adjusted to 10 with ammonia. The resulting solution was extracted with 6×150 mL of DCM:MeOH (10:1) and the organic layers combined and dried over anhydrous sodium sulfate. Concentration resulted in 1-(5-bromopyridin-2-yl)methanamine (18 g, 92% yield) as a white solid.

LCMS (ES, m/z): [M+H]⁺: 187

### Synthesis of Int. 7-c: N-[(5-bromopyridin-2-yl)methyl]formamide

Into a 500 mL 3-necked round-bottom flask was placed 1-(5-bromopyridin-2-yl)methanamine (18 g, 97 mmol, 1 equiv) and formic acid (150 mL). The resulting solution was stirred for 3 h at 100° C. in an oil bath, then cooled and diluted with 500 mL of H₂O. The resulting solution was extracted with 2×300 mL of ethyl acetate and the organic layers combined. The resulting solution was washed with 500 ml of brine, dried over anhydrous sodium sulphate and concentrated under vacuum. This resulted in N-[(5-bromopyridin-2-yl)methyl]formamide (13 g) as a crude brown solid.

LCMS (ES, m/z): [M+H]⁺: 215

### Synthesis of Int. 7-d: 6-bromoimidazo[1,5-a]pyridine

Into a 500 mL 3-necked round-bottom flask was placed N-[(5-bromopyridin-2-yl)methyl]formamide (13 g, 61 mmol, 1 equiv), toluene (150 mL) and POCl₃ (46.4 g, 305 mmol, 5 equiv). The resulting solution was stirred for 2 h at 100° C., then cooled and concentrated under vacuum. The residue was carefully diluted with 150 mL of H₂O and the pH adjusted to 10 with ammonia. The resulting solution was extracted with 3×150 mL of dichloromethane and the organic layers combined. The solution was washed with 300 ml of brine, dried over anhydrous sodium sulfate and concentrated under vacuum to give 6-bromoimidazo[1,5-a]pyridine (10.5 g, 79% yield) as a brown solid.

LCMS (ES, m/z): [M+H]⁺: 197

### Synthesis of Intermediate 7: 6-bromo-1-iodoimidazo[1,5-a]pyridine

Into a 250 mL 3-necked round-bottom flask was placed 6-bromoimidazo[1,5-a]pyridine (10.5 g, 53.5 mmol, 1 equiv) in DMF (100 mL). This was followed by the addition of NIS (12 g, 53.5 mmol, 1 equiv) at 0° C. The resulting solution was stirred for 60 min at 0° C. in an ice/salt bath, then quenched by the addition of 200 mL of saturated Na₂S₂O₃/H₂O. The solids were collected by filtration and dried to give 6-bromo-1-iodoimidazo[1,5-a]pyridine (12.8 g, 67% yield) as a brown solid.

LCMS (ES, m/z): [M+H]⁺: 323

### Example 8: Synthesis of 6-bromo-1-iodoimidazo[1,5-a]pyridine (Intermediate 8)

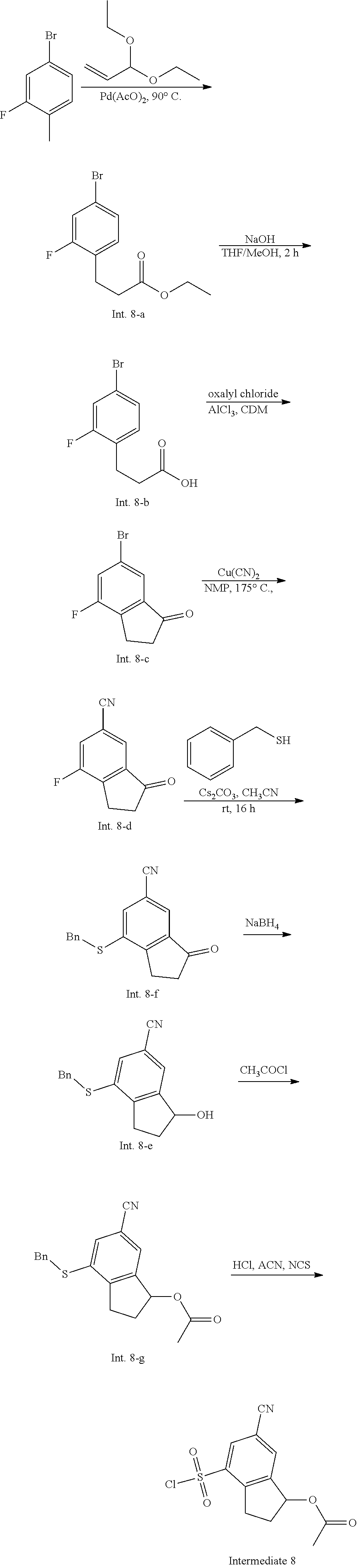

### Synthesis of Int. 8-a: Ethyl 3-(4-bromo-2-fluorophenyl)propanoate

Into a 500 mL 3-necked round-bottom flask, purged and maintained with an inert atmosphere of nitrogen, was placed 4-bromo-2-fluoro-1-iodobenzene (20 g, 67 mmol, 1 equiv), DMF (200 mL), 3,3-diethoxy-1-propene (11.3 g, 86 mmol, 1.3 equiv), tetrabutylammonium chloride (18.5 g, 67 mmol, 1 equiv), DIEA (23 g, 179 mmol, 2.7 equiv) and Pd(AcO)₂ (750 mg, 3 mmol, 0.05 equiv). The resulting solution was stirred for 2 h at 90° C. in an oil bath. The reaction mixture was cooled to 25° C. with a water/ice bath and the solution was diluted with 600 mL of H₂O. The resulting mixture was extracted with 2×200 mL of ethyl acetate and the organic layers combined. The organics were washed with 100 mL of brine. The mixture was dried over anhydrous sodium sulfate and concentrated under vacuum. The residue was applied onto a silica gel column, eluting with PE/EA=95/5. Ethyl 3-(4-bromo-2-fluorophenyl)propanoate (14.5 g, 50% yield) was isolated as a light yellow oil.

LCMS (ES, m/z): [M+H]⁺: 275.

### Synthesis of Int. 8-b: 3-(4-bromo-2-fluorophenyl)propanoic acid

Into a 1000 mL round-bottom flask, was placed ethyl 3-(4-bromo-2-fluorophenyl)propanoate (16.5 g, 60 mmol, 1 equiv), THE (120 mL), MeOH (120 mL) and 4N aqueous NaOH (120 mL, 480 mmol). The resulting solution was stirred for 2 h at 50° C. in an oil bath. The reaction mixture was concentrated under vacuum and the residue extracted with 2×100 mL of ethyl acetate. The pH of the aqueous layer was acidified with 4N HCl. The resulting suspension was extracted with 3×100 mL of ethyl acetate and the organic layers combined. The organics were washed with 100 mL of brine, dried over anhydrous sodium sulfate and concentrated under vacuum. The residue was applied onto a silica gel column, eluting with 0-40% EA/PE. 3-(4-Bromo-2-fluorophenyl) propanoic acid (11.5 g, 78% yield) was isolated as a white solid.

### Synthesis of Int. 8-c: 6-bromo-4-fluoro-2,3-dihydro-1H-inden-1-one

To a stirred mixture of 3-(4-bromo-2-fluorophenyl)propanoic acid (11.5 g, 47 mmol, 1 equiv) in DCM (200 mL) was added oxalyl chloride (11.8 g, 93 mmol, 2 equiv) dropwise at room temperature under nitrogen atmosphere. The resulting mixture was stirred for 4 h then concentrated under reduced pressure. The residue was dissolved in DCM (200 mL). To the above mixture was added AlCl₃ (18.6 g, 140 mmol, 3 equiv) in portions at room temperature. The resulting mixture was stirred for additional 3 h at 40° C. Further AlCl₃ (18.6 g, 140 mmol, 3 equiv) was added in portions. The resulting mixture was stirred overnight at 40° C. The reaction mixture was diluted with NH₄Cl (300 mL) and extracted with CH₂Cl₂ (3×200 mL). The combined organic layers were washed with brine (100 mL) and dried over anhydrous Na₂SO₄. After filtration, the filtrate was concentrated under reduced pressure. The residue was purified by silica gel column chromatography with EA/PE=1/2 to afford 6-bromo-4-fluoro-2,3-dihydroinden-1-one (6.5 g, 61% yield) as a white solid.

### Synthesis of Int. 8-d: 7-fluoro-3-oxo-2,3-dihydro-1H-indene-5-carbonitrile

Into a 100 mL round-bottom flask, purged and maintained with an inert atmosphere of nitrogen, was placed 6-bromo-4-fluoro-2,3-dihydroinden-1-one (4.6 g, 20.1 mmol, 1 equiv), NMP (50 mL) and Cu(CN)₂ (4.7 g, 40 mmol, 2 equiv). The resulting solution was stirred overnight at 175° C. The cooled reaction mixture was diluted with 200 mL of H₂O and extracted with 3×50 mL of ethyl acetate. The combined extracts were washed with 100 mL of brine, dried over anhydrous sodium sulfate and concentrated under vacuum. The residue was applied onto a silica gel column, eluting with 50-70% THF/PE. 7-Fluoro-3-oxo-1,2-dihydroindene-5-carbonitrile (1.5 g, 43% yield) was isolated as a light yellow solid.

### Synthesis of Int. 8-e: 7-(benzylthio)-3-oxo-2,3-dihydro-1H-indene-5-carbonitrile

Into a 4 mL vial was added 7-fluoro-3-oxo-1,2-dihydroindene-5-carbonitrile (450 mg, 2.6 mmol, 1 equiv) and ACN (15 mL) at room temperature. To the stirred solution was added Cs₂CO₃ (920 mg, 2.8 mmol, 1.1 equiv) and benzyl mercaptan (478 mg, 3.9 mmol, 1.5 equiv) at room temperature. The resulting mixture was stirred overnight at room temperature. The reaction mixture was concentrated under reduced pressure. The residue was purified by silica gel column chromatography, eluting with CH₂Cl₂/MeOH (9:1) to afford 7-(benzylsulfanyl)-3-oxo-1,2-dihydroindene-5-carbonitrile (550 mg, 77% yield) as a white solid.

### Synthesis of Int. 8-f: 7-(benzylsulfanyl)-3-hydroxy-2,3-dihydro-1H-indene-5-carbonitrile

Into a 100 mL round-bottom flask were added 7-(benzylsulfanyl)-3-oxo-1,2-dihydroindene-5-carbonitrile (550 mg, 2 mmol, 1 equiv) and MeOH (15 mL). To the solution was added NaBH₄ (97 mg, 2.6 mmol, 1.3 equiv) at room temperature. The resulting mixture was stirred for 1 h at room temperature, then diluted with water (50 mL). The resulting mixture was extracted with EtOAc (3×50 mL). The combined organic layers were washed with brine (30 mL) and dried over anhydrous Na₂SO₄. After filtration, the filtrate was concentrated under reduced pressure. The residue was purified by silica gel column chromatography, eluting with CH₂Cl₂/MeOH (9:1) to afford 7-(benzylsulfanyl)-3-hydroxy-2,3-dihydro-1H-indene-5-carbonitrile (560 mg, crude) as a light grey solid.

### Synthesis of Int. 8-g: 4-(benzylsulfanyl)-6-cyano-2,3-dihydro-1H-inden-1-yl acetate

Into a 2 mL vial were added 7-(benzylsulfanyl)-3-hydroxy-2,3-dihydro-1H-indene-5-carbonitrile (400 mg, 1.4 mmol, 1 equiv) and DCM (10 mL). To the stirred solution was added TEA (288 mg, 2.8 mmol, 2 equiv) and acetyl chloride (167 mg, 2.1 mmol, 1.5 equiv) dropwise at room temperature under nitrogen atmosphere. The reaction mixture was stirred for 1.5 h at room temperature, then quenched by the addition of MeOH (5 mL). The resulting mixture was concentrated under reduced pressure. The residue was purified by silica gel column chromatography, eluting with PE/EtOAc (8:1) to afford 4-(benzylsulfanyl)-6-cyano-2,3-dihydro-1H-inden-1-yl acetate (360 mg, 78% yield) as a white solid.

### Synthesis of Intermediate 8: 4-(chlorosulfonyl)-6-cyano-2,3-dihydro-1H-inden-1-yl acetate

Into a 20 mL vial were added 4-(benzylsulfanyl)-6-cyano-2,3-dihydro-1H-inden-1-yl acetate (400 mg, 1.2 mmol, 1 equiv) and MeCN (4 mL). To the stirred mixture was added 1M HCl (1.2 mL, 33 mmol, 32 equiv) and NCS (661 mg, 4.8 mmol, 4 equiv) in portions. The resulting mixture was diluted with water (10 mL) when the reaction was shown to be complete. The resulting mixture was extracted with EtOAc (3×10 mL). The combined organic layers were washed with brine (5 mL) and dried over anhydrous Na₂SO₄. After filtration, the filtrate was concentrated under reduced pressure. The crude product was used in the next step directly without further purification.

### Example 9: Synthesis of ethyl 2-bromoimidazo[1,5-b]pyridazine-5-carboxylate (Intermediate 9)

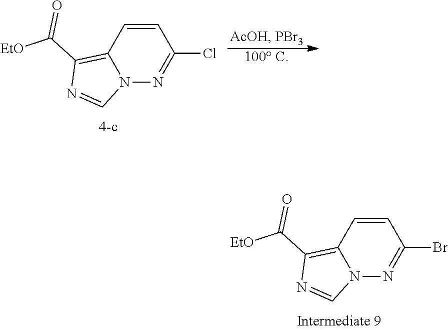 [SMILES: CCOC(=O)c1ncn2nc(Cl)ccc12 ; CCOC(=O)c1ncn2nc(Br)ccc12]

### Synthesis of Int. 9: ethyl 2-bromoimidazo[1,5-b]pyridazine-5-carboxylate

Into a 10 L 3-necked round-bottom flask, was placed a solution of ethyl 2-chloroimidazo[1,5-b]pyridazine-5-carboxylate (500 g, 2200 mmol, 1 equiv) in AcOH (5 L) and PBr₃ (1800 g, 6650 mmol, 3 equiv). The resulting solution was stirred overnight at 100° C., then quenched by the addition of water/ice. The resulting solution was extracted with ethyl acetate (3×2 L and the combined organic layers were treated with ammonia until the pH was 8. The resulting mixture was washed with brine (5 L), then concentrated to give ethyl 2-bromoimidazo[1,5-b]pyridazine-5-carboxylate (351 g, 59% yield) as a yellow solid.

LC-MS: (ES, m z): [M+H]⁺: 270

1H NMR (400 MHz, DMSO-d₆, ppm): δ 8.85 (s, 1H), 8.34 (d, J=9.5 Hz, 1H), 7.36 (d, J=9.5 Hz, 1H), 4.35 (q, J=7.1 Hz, 2H), 1.35 (t, J=7.1 Hz, 3H).

### Example 10: Synthesis of 3-cyano-5-(trifluoromethyl)benzenesulfonyl chloride (Intermediate 10)

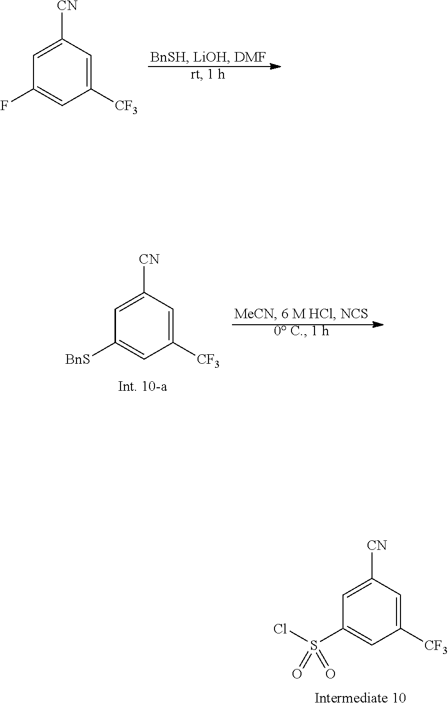

### Synthesis of Int. 10-a: 3-(benzylsulfanyl)-5-(trifluoromethyl)benzonitrile

Into a 250 mL round-bottom flask, was placed 3-fluoro-5-(trifluoromethyl)benzonitrile (2 g, 10 mmol, 1 equiv), DMF (50 mL), LiOH (0.5 g, 21 mmol, 2 equiv) and benzyl mercaptan (1.6 g, 13 mmol, 1.2 equiv). The resulting solution was stirred for 1 h at 25° C., then quenched by the addition of 100 mL of water. The resulting solution was extracted with 2×100 mL of ethyl acetate, and the combined organics washed with 3×100 mL of water. The organics were dried over anhydrous sodium sulfate and concentrated, to give 3-(benzylsulfanyl)-5-(trifluoromethyl)benzonitrile (3 g, 97% yield) as a yellow oil, which was used in next step directly without further purification.

### Synthesis of Intermediate 10: 3-cyano-5-(trifluoromethyl)benzenesulfonyl chloride

Into a 40 mL vial, was placed MeCN (7.5 mL) and HCl (aqueous, 6M) (1.5 mL). This was followed by the addition of NCS (910 mg, 6.8 mmol, 4 equiv), in portions at 0° C. To this was added 3-(benzylsulfanyl)-5-(trifluoromethyl)benzonitrile (500 mg, 1.7 mmol, 1 equiv), in portions at 0° C. The resulting solution was stirred for 1 h in a water/ice bath. The reaction was quenched by the addition of 20 mL of water/ice and the resulting solution extracted with 2×20 mL of dichloromethane. The combined organics were washed with 2×20 mL of water, dried over anhydrous sodium sulfate and concentrated. This resulted in 3-cyano-5-(trifluoromethyl)benzenesulfonyl chloride (300 mg) as a crude yellow oil which was used in next step directly without further purification.

### Example 11: Synthesis of 6-(3-amino-2,6-difluorophenyl)imidazo[1,5-a]pyrazine-1-carboxylic acid & 2,4-difluoro-3-[1-iodoimidazo[1,5-a]pyrazin-6-yl]aniline (Intermediate 11 and Intermediate 12)

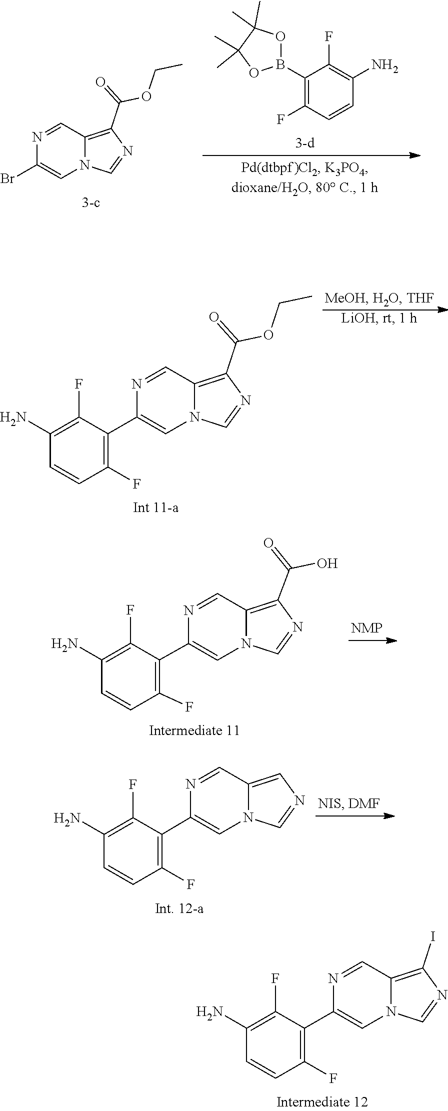

### Synthesis of Int. 11-a: 6-(3-amino-2,6-difluorophenyl)imidazo[1,5-a]pyrazine-1-carboxylate

To a solution of ethyl 6-bromoimidazo[1,5-a]pyrazine-1-carboxylate (120 g, 444 mmol, 1 equiv) and 2,4-difluoro-3-(4,4,5,5-tetramethyl-1,3,2-dioxaborolan-2-yl)aniline (170 g, 666 mmol, 1.5 equiv) in dioxane (1 L) and H₂O (200 mL) were added K₃PO₄ (189 g, 889 mmol, 2 equiv) and Pd(dtbpf)Cl₂ (29 g, 44 mmol, 0.1 equiv). After stirring for 3 h at 90° C. under nitrogen, the resulting mixture was concentrated under reduced pressure. The residue was purified by silica gel column chromatography, eluting with PE/EtOAc (5:1) to afford ethyl 6-(3-amino-2,6-difluorophenyl)imidazo[1,5-a]pyrazine-1-carboxylate (110 g, 78% yield) as an off-white solid.

LCMS (ES, m/z): [M+H]⁺: 319

### Synthesis of Intermediate 11: 6-(3-amino-2,6-difluorophenyl)imidazo[1,5-a]pyrazine-1-carboxylic acid

To a stirred solution of ethyl 6-(3-amino-2,6-difluorophenyl)imidazo[1,5-a]pyrazine-1-carboxylate (110 g, 345 mmol, 1 equiv) in MeOH (500 mL), H₂O (500 mL) and THF (500 mL), was added LiOH (25 g, 1040 mmol, 3 equiv) in portions at room temperature. The resulting mixture was stirred for 1 h then concentrated under vacuum. The resulting mixture was diluted with water (100 mL) and acidified to pH 3 with HCl (aq.). The precipitated solids were collected by filtration and washed with water (3×50 mL). Drying gave 6-(3-amino-2,6-difluorophenyl)imidazo[1,5-a]pyrazine-1-carboxylic acid (100 g, 99% yield) as a white solid.

LCMS (ES, m/z): [M+H]⁺: 291

### Synthesis of Int. 12-a: 2,4-difluoro-3-[imidazo[1,5-a]pyrazin-6-yl]aniline

Into a 2 L sealed tube were added 6-(3-amino-2,6-difluorophenyl)imidazo[1,5-a]pyrazine-1-carboxylic acid (100 g, 344 mmol, 1 equiv) and NMP (1.5 L) at room temperature. The resulting mixture was stirred for 3 h at 200° C. The residue was purified by silica gel column chromatography, eluting with PE/EtOAc (1:1) to afford 2,4-difluoro-3-[imidazo[1,5-a]pyrazin-6-yl]aniline (50 g, 59% yield) as a white solid.

LCMS (ES, m/z): [M+H]⁺: 247

### Synthesis of Intermediate 12: 2,4-difluoro-3-[1-iodoimidazo[1,5-a]pyrazin-6-yl]aniline

To a stirred solution of 2,4-difluoro-3-[imidazo[1,5-a]pyrazin-6-yl]aniline (50 g, 203 mmol, 1 equiv) in DMF (1 L) was added NIS (54.8 g, 243 mmol, 1.2 equiv) in portions. The reaction was stirred for 3 h then quenched by the addition of water (500 mL). The resulting mixture was extracted with EtOAc (3×200 mL). The combined organics were washed with brine (2×500 mL), dried over anhydrous Na₂SO₄ and concentrated under reduced pressure.

The residue was purified by silica gel column chromatography, eluting with PE/EtOAc (1:1) to afford 2,4-difluoro-3-[1-iodoimidazo[1,5-a]pyrazin-6-yl]aniline (35 g, 46% yield) as an off-white solid.

LCMS (ES, m/z): [M+H]⁺: 373 H NMR (300 MHz, DMSO-d₆) δ 8.90 (d, J=1.6 Hz, 1H), 8.63 (d, J=0.7 Hz, 1H), 8.55 (s, 1H), 7.00-6.78 (m, 2H), 5.14 (s, 2H).

### Example 12: Synthesis of methyl 6-bromoimidazo[1,5-a]pyridine-1-carboxylate (Intermediate 13)

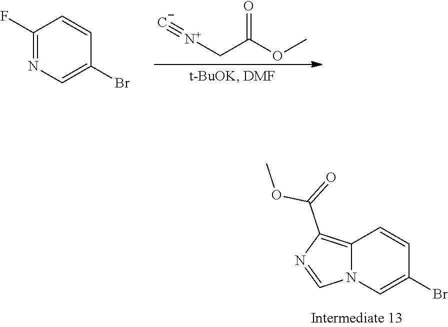

### Synthesis of Intermediate 13: methyl 6-bromoimidazo[1,5-a]pyridine-1-carboxylate

To a stirred mixture of 5-bromo-2-fluoropyridine (130 g, 739 mmol, 1 equiv) and methyl 2-isocyanoacetate (88 g, 886 mmol, 1.2 equiv) in DMF (4 L) was added t-BuOK (887 mL, 1.2 equiv, 1 mol/L in THF) dropwise at 0° C. under nitrogen atmosphere. The mixture was stirred for 3 h, then was poured into water/ice (1 L) and the resulting mixture extracted with CH₂Cl₂ (4×1 L). The combined organics were washed with brine (2×1 L), dried over anhydrous Na₂SO₄ and concentrated under reduced pressure. The residue was purified by silica gel column chromatography, eluting with PE/EtOAc (1:1) to afford methyl 6-bromoimidazo[1,5-a]pyridine-1-carboxylate (60 g, 32% yield) as a white solid.

LCMS (ES, m/z): [M+H]⁺: 255, 257

¹H NMR (300 MHz, DMSO-d₆) δ 8.90 (dd, J=1.7, 1.0 Hz, 1H), 8.45-8.39 (m, 1H), 7.92 (dt, J=9.6, 0.9 Hz, 1H), 7.33 (dd, J=9.6, 1.7 Hz, 1H), 3.84 (s, 3H).

### Example 13: Synthesis of methyl 6-(3-amino-2,6-difluorophenyl)imidazo[1,5-a]pyridine-1-carboxylate (Intermediate 14)

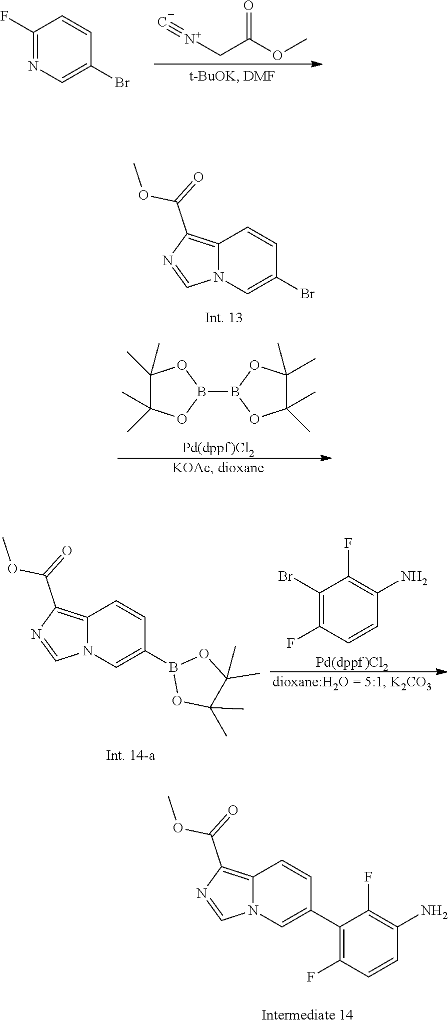

### Synthesis of Intermediate 14: methyl 6-(3-amino-2,6-difluorophenyl)imidazo[1,5-a]pyridine-1-carboxylate

Into a 1 L 3-necked round-bottom flask, purged and maintained with an inert atmosphere of nitrogen, was placed methyl 6-bromoimidazo[1,5-a]pyridine-1-carboxylate (40 g, 157 mmol, 1 equiv), bis(pinacolato)diboron (47.8 g, 188 mmol, 1.2 equiv), dioxane (500 mL), KOAc (31 g, 314 mmol, 2 equiv) and Pd(dppf)Cl₂ (11.5 g, 16 mmol, 0.1 equiv) at room temperature. The resulting solution was stirred for 1 h at 90° C. under nitrogen atmosphere.

The reaction mixture was cooled to room temperature and H₂O (100 mL), K₂CO₃ (43 g, 311 mmol, 2 equiv), 3-bromo-2,4-difluoroaniline (48.5 g, 233 mmol, 1.5 equiv) and Pd(dppf)Cl₂ (11.4 g, 15.6 mmol, 0.1 equiv) were added. The resulting mixture was stirred for 1 h at 80° C. under nitrogen atmosphere, then concentrated under reduced pressure. The residue was applied onto a silica gel column, eluting with ethyl acetate/petroleum ether (4:1) to afford methyl 6-(3-amino-2,6-difluorophenyl)imidazo[1,5-a]pyridine-1-carboxylate (32 g, 68% yield) as a white solid.

LCMS (ES, m/z): [M+H]⁺: 304

### Example 14: Synthesis of ethyl 6-bromoimidazo[1,5-a]pyrazine-1-carboxylate (Intermediate 15)

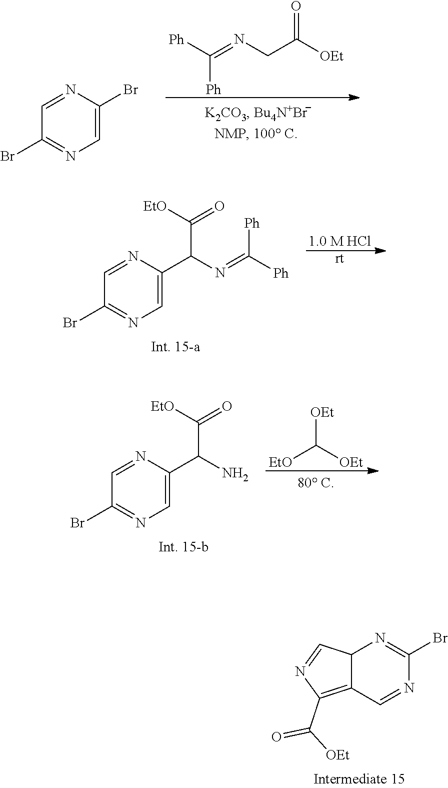 [SMILES: CCOC(=O)CN=C(c1ccccc1)c1ccccc1 ; Brc1cnc(Br)cn1 ; CCOC(=O)C(N=C(c1ccccc1)c1ccccc1)c1cnc(Br)cn1 ; CCOC(=O)C(N)c1cnc(Br)cn1 ; CCOC(OCC)OCC ; CCOC(=O)C1=C2C=NC(Br)=NC2C=N1]

### Synthesis of Int. 15-a: ethyl 2-(5-bromopyrazin-2-yl)-2-[(diphenylmethylidene)amino]acetate

Into a 50 L 4-necked round-bottom flask was placed a solution of 2,5-dibromopyrazine (1.3 kg, 5465 mmol, 1 equiv) in NMP (25 L), K₂CO₃ (1522 g, 10930 mmol, 2 equiv), ethyl 2-[(diphenylmethylidene)amino]acetate (1534 g, 5738 mmol, 1.05 equiv) and tetrabutylammonium bromide (1762 g, 5465 mmol, 1 equiv). The resulting solution was stirred for 10 h at 100° C. The reaction was cooled and quenched by the addition of 20 L of water/ice. The resulting solution was extracted with 3×15 L of ethyl acetate and the organic layers combined. The resulting mixture was washed with 1×10 L of brine, then concentrated under vacuum. This resulted in crude ethyl 2-(5-bromopyrazin-2-yl)-2-[(diphenylmethylidene)amino]acetate (2710 g) as a brown solid.

LC-MS: (ES, m z): [M+H] 424

### Synthesis of Int. 15-b: ethyl 2-amino-2-(5-bromopyrazin-2-yl)acetate

Into a 50 L 4-necked round-bottom flask was placed a solution of ethyl 2-(5-bromopyrazin-2-yl)-2-[(diphenylmethylidene)amino]acetate (2500 g, purity 60%) in THE (10 L) and HCl (1 M, 10 L). The resulting solution was stirred for 30 min at 10° C. The reaction was washed with dichloromethane (2×20 L), and the pH adjusted to 8 with NH₄OH. The resulting solution was extracted with 3×15 L of dichloromethane and the organic layers combined. The organics were washed with 10 L of brine and concentrated under vacuum. Crude ethyl 2-amino-2-(5-bromopyrazin-2-yl)acetate (1000 g) was obtained as a yellow solid.

LC-MS: (ES, m z): [M+H] 260

### Synthesis of Intermediate 15: ethyl 6-bromoimidazo[1,5-a]pyrazine-1-carboxylate

Into a 10 L 4-necked round-bottom flask was placed ethyl 2-amino-2-(5-bromopyrazin-2-yl)acetate (1 kg, 3076 mmol, 1 equiv, 80%) and triethyl orthoformate (5 L). The resulting solution was stirred for 1 h at 80° C., then cooled to 0° C. with a water/ice bath. The solids were collected by filtration and dried to give ethyl 6-bromoimidazo[1,5-a]pyrazine-1-carboxylate (351 g, 42% yield) as a pink solid.

LCMS (ES, m z): [M+H] 270

¹H NMR: (300 MHz, DMSO-d₆, ppm): δ 9.21 (dd, J=1.6, 0.6 Hz, 1H), 8.87 (d, J=1.5 Hz, 1H), 8.57 (d, J=0.6 Hz, 1H), 4.36 (q, J=7.1 Hz, 2H), 1.35 (t, J=7.1 Hz, 3H).

### Example 15: Synthesis of ethyl 6-(3-amino-2,6-difluorophenyl)imidazo[1,5-a]pyridine-1-carboxylate (Intermediate 16)

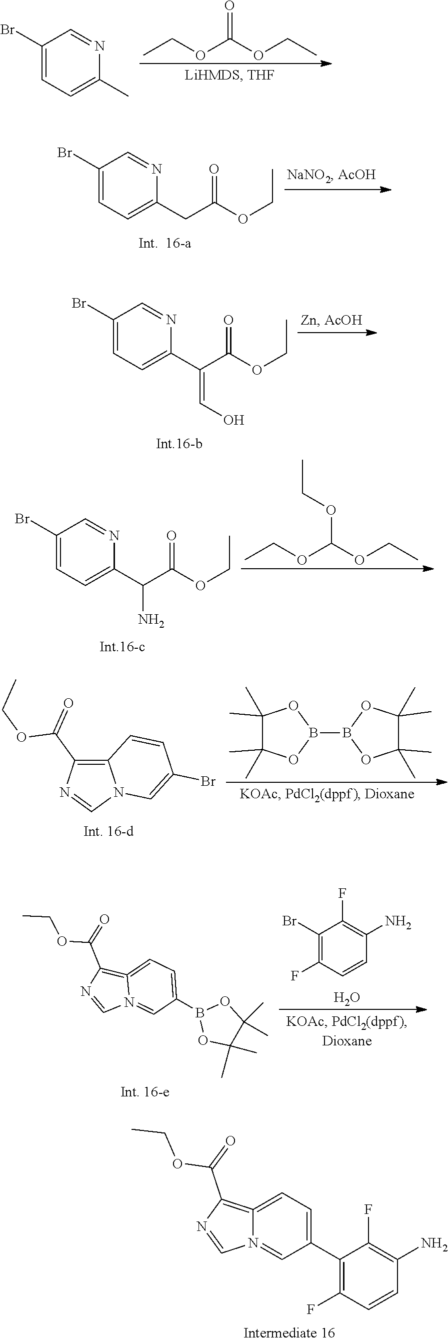

### Synthesis of Int. 16-a: ethyl 2-(5-bromopyridin-2-yl)acetate

Into a 20 L 4-necked round-bottom flask purged and maintained with an inert atmosphere of nitrogen, was placed a solution of 5-bromo-2-methylpyridine (300 g, 1744 mmol, 1 equiv) in THE (10.5 L). The solution was cooled to 0° C. and 1M in THE solution of LiHMDS (4 L) was added dropwise with stirring at 0° C. The solution was stirred at low temperature for 30 mins, then diethyl carbonate (311 g, 2633 mmol, 1.5 equiv) was added. The resulting solution was stirred for 5 h at room temperature. The reaction solution was extracted with 5 L of ethyl acetate and the organics dried over anhydrous sodium sulfate. Concentration gave ethyl 2-(5-bromopyridin-2-yl)acetate (400 g, 94% yield) as a black oil.

### Synthesis of Int. 16-b: ethyl (2Z)-2-(5-bromopyridin-2-yl)-2-(N-hydroximino)acetate

Into a 5 L 4-necked round-bottom flask, purged and maintained with an inert atmosphere of nitrogen, was placed ethyl 2-(5-bromopyridin-2-yl)acetate (400 g, 1639 mmol, 1 equiv) and AcOH (800 mL). After cooling to 0° C. a solution of NaNO₂ (114 g, 1652 mmol, 1.01 equiv) in H₂O (280 mL) was added dropwise with stirring. The resulting solution was stirred for 1 h at room temperature. The reaction was quenched by the addition of 1 L of water, and the resulting solution was extracted with 1 L of ethyl acetate. The organics were dried over anhydrous sodium sulfate and concentrated. This gave ethyl (2Z)-2-(5-bromopyridin-2-yl)-2-(N-hydroxyimino)acetate (450 g) as a crude black oil.

### Synthesis of Int. 16-c: ethyl 2-amino-2-(5-bromopyridin-2-yl)acetate

Into a 10 L 4-necked round-bottom flask, purged and maintained with an inert atmosphere of nitrogen, was placed ethyl (2Z)-2-(5-bromopyridin-2-yl)-2-(N-hydroxyimino)acetate (450 g, 1648 mmol, 1 equiv) and AcOH (4.5 L). The mixture was cooled to 0° C. and Zn (318 g, 4862 mmol, 2.95 equiv) was added. The resulting mixture was stirred for 1 h at room temperature. The solids were removed by filtration and the filtrate concentrated to give ethyl 2-amino-2-(5-bromopyridin-2-yl)acetate (379 g, 89% yield) as a black oil.

### Synthesis of Int. 16-d: ethyl 6-bromoimidazo[1,5-a]pyridine-1-carboxylate

Into a 2 L 4-necked round-bottom flask, was placed ethyl 2-amino-2-(5-bromopyridin-2-yl)acetate (379 g, 1463 mmol, 1 equiv) and triethyl orthoformate (645 mL). The resulting solution was stirred for 10 min at 130° C., then concentrated. The residue was applied onto a silica gel column, eluting with ethyl acetate/petroleum ether (1:3). The collected fractions were combined and concentrated to give ethyl 6-bromoimidazo[1,5-a]pyridine-1-carboxylate (125 g, 32% yield) as a black oil.

### Synthesis of Int. 16-e: ethyl 6-(4,4,5,5-tetramethyl-1,3,2-dioxaborolan-2-yl)imidazo[1,5-a]pyridine-1-carboxylate

Into a 5 L 4-necked round-bottom flask purged and maintained with an inert atmosphere of nitrogen, was placed ethyl 6-bromoimidazo[1,5-a]pyridine-1-carboxylate (125 g, 465 mmol, 1 equiv), dioxane (3125 mL), bis(pinacolato)diboron (178 g, 700 mmol, 1.5 equiv), Pd(dppf)Cl₂ (38 g, 52 mmol, 0.11 equiv) and KOAc (138 g, 1406 mmol, 3.03 equiv). The resulting solution was stirred for 1 h at 85° C., then concentrated. The residue was suspended in 1 L of EA and the solids removed by filtration. The filtrate was concentrated to give ethyl 6-(4,4,5,5-tetramethyl-1,3,2-dioxaborolan-2-yl)imidazo[1,5-a]pyridine-1-carboxylate (150 g) as a crude black solid.

### Synthesis of Intermediate 16: ethyl 6-(3-amino-2,6-difluorophenyl)imidazo[1,5-a]pyridine-1-carboxylate

Into a 10 L 4-necked round-bottom flask, purged and maintained with an inert atmosphere of nitrogen, was placed ethyl 6-(4,4,5,5-tetramethyl-1,3,2-dioxaborolan-2-yl)imidazo[1,5-a]pyridine-1-carboxylate (100 g, 316 mmol, 1 equiv), 3-bromo-2,4-difluoroaniline (65.5 g, 315 mmol, 1 equiv), dioxane (5 L), Pd(dppf)Cl₂·CH₂Cl₂ (25.8 g, 35 mmol, 0.11 equiv), K₂CO₃ (131 g, 947 mmol, 2.99 equiv) and H₂O (1 L). The resulting solution was stirred for 2 h at 85° C., then concentrated. The resulting mixture was extracted with 1 L of ethyl acetate and the organics concentrated. The residue was applied onto a silica gel column, eluting with ethyl acetate/petroleum ether (1:4). The collected fractions were combined and concentrated to give ethyl 6-(3-amino-2,6-difluorophenyl)imidazo[1,5-a]pyridine-1-carboxylate (53 g, 53% yield) as a brown solid.

LCMS (ES, m/z): 318[M+H]+

1H NMR (300 MHz, Methanol-d4, ppm): δ 8.58 (s, 1H), 8.46 (s, 1H), 8.16 (d, J=9.4 Hz, 1H), 7.33 (d, J=9.5 Hz, 1H), 6.96-6.83 (m, 2H), 4.45 (q, J=7.1 Hz, 2H), 3.21 (s, 1H), 1.45 (t, J=7.1 Hz, 3H), 1.20 (s, 1H).

### Example 16: Synthesis of 6-[3-(5-chloro-2-methoxypyridine-3-sulfonamido)-2,6-difluorophenyl]-N-methylimidazo[1,5-a]pyridine-1-carboxamide (Compound 1)

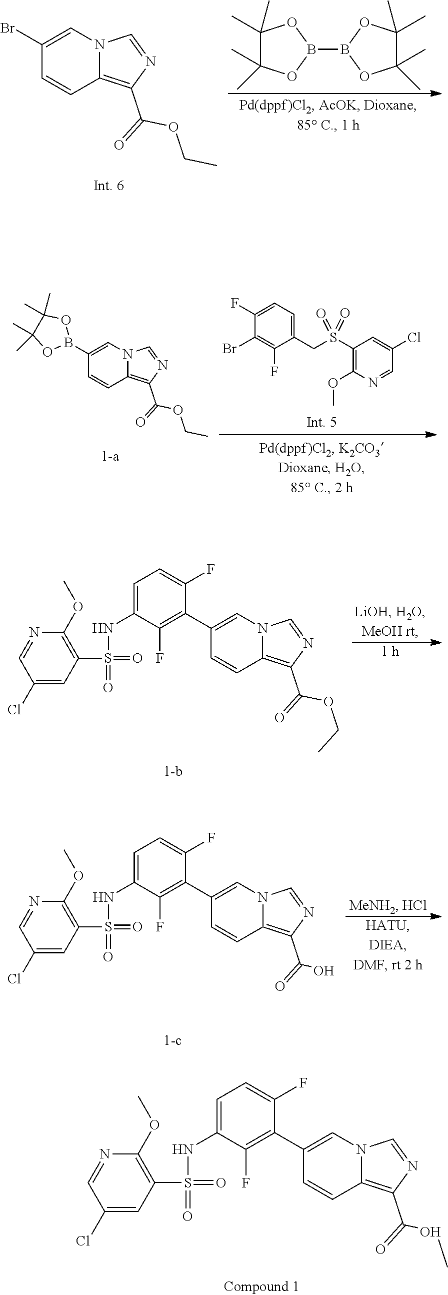

### Synthesis of 1-a: ethyl 6-(4,4,5,5-tetramethyl-1,3,2-dioxaborolan-2-yl)imidazo[1,5-a]pyridine-1-carboxylate

Ethyl 6-bromoimidazo[1,5-a]pyridine-1-carboxylate (200 mg, 0.75 mmol, 1 equiv), bis(pinacolato)diboron (284 mg, 1.1 mmol, 1.5 equiv), Pd(dppf)Cl₂ (55 mg, 0.075 mmol, 0.1 equiv) and AcOK (219 mg, 2.24 mmol, 3 equiv) were suspended in dioxane (5 mL). The resulting solution was stirred for 1 h at 85° C. in an oil bath, then cooled and concentrated. The residue was applied to a silica gel column and eluted with ethyl acetate/petroleum ether (1:10). Ethyl 6-(4,4,5,5-tetramethyl-1,3,2-dioxaborolan-2-yl)imidazo[1,5-a]pyridine-1-carboxylate (188 mg) was isolated as a white solid.

LCMS (ES, m/z): [M+H]⁺: 317

### Synthesis of 1-b: ethyl 6-[3-(5-chloro-2-methoxypyridine-3-sulfonamido)-2,6-difluorophenyl]imidazo[1,5-a]pyridine-1-carboxylate

Ethyl 6-(4,4,5,5-tetramethyl-1,3,2-dioxaborolan-2-yl)imidazo[1,5-a]pyridine-1-carboxylate (188 mg, 0.6 mmol, 1 equiv), N-(3-bromo-2,4-difluorophenyl)-5-chloro-2-methoxypyridine-3-sulfonamide (246 mg, 0.6 mmol, 1 equiv), Pd(dppf)Cl₂ (44 mg, 0.06 mmol, 0.1 equiv) and K₂CO₃ (246 mg, 1.8 mmol, 3 equiv) were suspended in dioxane (10 mL) and H₂O (2 mL). The resulting solution was stirred for 2 h at 85° C. in an oil bath. The reaction mixture was cooled to room temperature and concentrated. The residue was applied onto a silica gel column and eluted with ethyl acetate/petroleum ether (1:1). Ethyl 6-[3-(5-chloro-2-methoxypyridine-3-sulfonamido)-2,6-difluorophenyl]imidazo[1,5-a]pyridine-1-carboxylate (155 mg) was isolated as a white solid.

LCMS (ES, m/z): [M+H]⁺: 523

### Synthesis of 1-c: 6-[3-(5-chloro-2-methoxypyridine-3-sulfonamido)-2,6-difluorophenyl]imidazo[1,5-a]pyridine-1-carboxylic acid

Ethyl 6-[3-(5-chloro-2-methoxypyridine-3-sulfonamido)-2,6-difluorophenyl]imidazo[1,5-a]pyridine-1-carboxylate (155 mg, 0.3 mmol, 1 equiv) in MeOH (10 mL) was treated with a solution of LiOH (22 mg, 0.9 mmol, 3 equiv) in H₂O (2 mL) dropwise with stirring at room temperature. The resulting solution was stirred for 1 h at room temperature, then concentrated. The residue was diluted with 10 mL of H₂O, and the pH adjusted to 5-6 with HCl (2 mol/L). The resulting solution was extracted with 2×10 mL of ethyl acetate. The organic solution was washed with 20 ml of brine and dried over anhydrous sodium sulfate. The filtrate was concentrated under vacuum to give 6-[3-(5-chloro-2-methoxypyridine-3-sulfonamido)-2,6-difluorophenyl]imidazo[1,5-a]pyridine-1-carboxylic acid (103 mg) as a white solid which was used in next step directly without further purification.

LCMS (ES, m/z): [M+H]⁺: 495

### Synthesis of Compound 1: 6-[3-(5-chloro-2-methoxypyridine-3-sulfonamido)-2,6-difluorophenyl]-N-methylimidazo[1,5-a]pyridine-1-carboxamide

6-[3-(5-Chloro-2-methoxypyridine-3-sulfonamido)-2,6-difluorophenyl]imidazo[1,5-a]pyridine-1-carboxylic acid (103 mg, 0.21 mmol, 1 equiv), CH₃NH₂·H₂O (14 mg, 0.21 mmol, 1 equiv), HATU (116 mg, 0.31 mmol, 1.5 equiv) and DIEA (40 mg, 0.31 mmol, 1.5 equiv) were dissolved in DMF (5 mL). The resulting solution was stirred for 2 h at room temperature. Concentration gave the crude product (150 mg) which was purified by Flash-Prep-HPLC using the following conditions: (IntelFlash-1): welch Ultimate XB-C18, 50×250 mm, 10 μm mobile phase, Mobile Phase A: Water (0.05% NH₃·H₂O), Mobile Phase B: ACN, Gradient: 10-35% B; Detector, 220 nm. 6-[3-(5-Chloro-2-methoxypyridine-3-sulfonamido)-2,6-difluorophenyl]-N-methylimidazo[1,5-a]pyridine-1-carboxamide (23 mg) was isolated as a white solid.

LCMS (ES, m/z): [M+H]⁺: 508

¹H NMR (300 MHz, DMSO-d₆) δ 10.46 (s, 1H), 8.60 (s, 1H), 8.54-8.46 (m, 2H), 8.19-8.05 (m, 3H), 7.44-7.30 (m, 1H), 7.24 (t, J=9.2 Hz, 1H), 7.02 (d, J=9.5 Hz, 1H), 3.92 (s, 3H), 2.81 (d, J=4.7 Hz, 3H).

### Example 17: Synthesis of 5-chloro-N-[2,4-difluoro-3-[1-(1H-imidazol-2-yl)imidazo[1,5-a]pyridin-6-yl]phenyl]-2-methoxypyridine-3-sulfonamide (Compound 2)

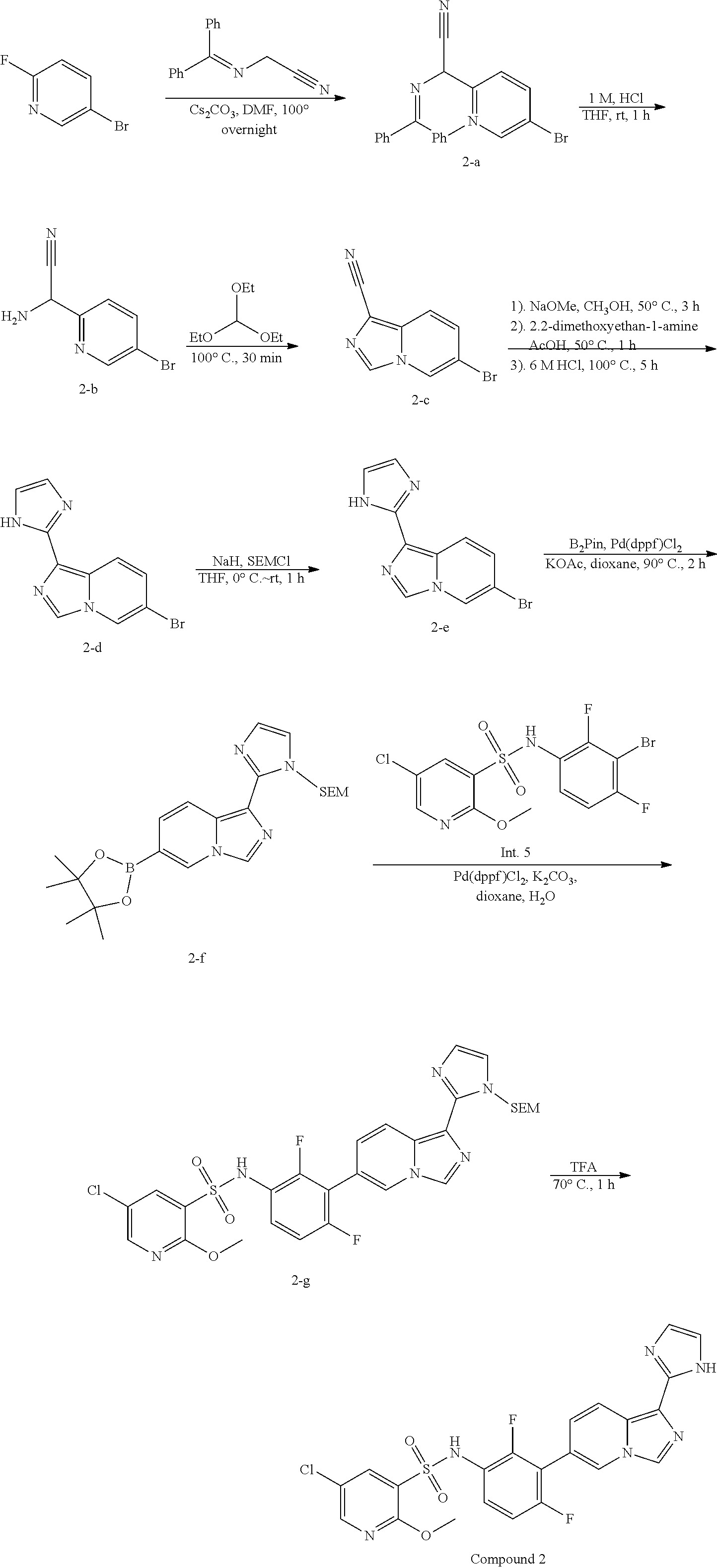

### Synthesis of 2-a: 2-(5-bromopyridin-2-yl)-2-[(diphenylmethylidene)amino]acetonitrile

A solution of 5-bromo-2-fluoropyridine (50 g, 284 mmol, 1 equiv), Cs₂CO₃ (278 g, 852 mmol, 3 equiv) and 2-[(diphenylmethylidene)amino]acetonitrile (62.6 g, 284 mmol, 1 equiv) in DMF (1000 mL) was stirred overnight at 100° C. The mixture was allowed to cool to room temperature and diluted with water (3000 mL), before being extracted with EA (3×1000 mL). The combined organic layers were washed with brine (2×500 mL) and dried over anhydrous Na₂SO₄. After filtration, the filtrate was concentrated under reduced pressure. The residue was purified by silica gel column chromatography, eluting with PE/EA (3/1) to afford 2-(5-bromopyridin-2-yl)-2-[(diphenylmethylidene)amino]acetonitrile (75 g, 70% yield) as a pink oil.

LCMS (ES, m/z): [M+H]⁺: 376

### Synthesis of 2-b: 2-amino-2-(5-bromopyridin-2-yl)acetonitrile

To a stirred solution of 2-(5-bromopyridin-2-yl)-2-[(diphenylmethylidene)amino]acetonitrile (75 g, 199 mmol, 1 equiv) in THF (400 mL) was added 2 M hydrochloric acid (300 mL) dropwise at 0° C. The resulting mixture was stirred for 1 h at room temperature, then concentrated under reduced pressure to remove THF. The mixture was basified to pH 8 with saturated aqueous NaHCO₃. The resulting mixture was extracted with EA (3×500 mL). The combined organic layers were washed with brine (300 mL) and dried over anhydrous Na₂SO₄. After filtration, the filtrate was concentrated under reduced pressure to give 2-amino-2-(5-bromopyridin-2-yl)acetonitrile (23.9 g, crude) as a yellow oil which was used in the next step directly without further purification.

LCMS (ES, m/z): [M+H]⁺: 212

### Synthesis of 2-c: 6-bromoimidazo[1,5-a]pyridine-1-carbonitrile

A solution of 2-amino-2-(5-bromopyridin-2-yl)acetonitrile (23.9 g, 113 mmol, 1 equiv) in triethyl orthoformate (100 mL) was stirred for 30 min at 100° C. The mixture was cooled to 0° C. and the solid formed collected by filtration and dried under vacuum to give 6-bromoimidazo[1,5-a]pyridine-1-carbonitrile (14 g, 56% yield) as a yellow solid.

LCMS (ES, m/z): [M+H]⁺: 222

### Synthesis of 2-d: 2-[6-bromoimidazo[1,5-a]pyridin-1-yl]-1H-imidazole

To a stirred solution of 6-bromoimidazo[1,5-a]pyridine-1-carbonitrile (14 g, 63 mmol, 1 equiv) in CH₃OH (100 mL) was added NaOMe (13.6 g, 252 mmol, 4 equiv) in portions at 0° C. The resulting mixture was stirred for 3 h at 50° C. then cooled to room temperature. To this was added 2,2-dimethoxyethan-1-amine (5 g, 47 mmol, 1.2 equiv) and AcOH (11.8 g, 197 mmol, 5 equiv), and the mixture was stirred for 1 h at 50° C. After cooling to room temperature, MeOH (168 mL) was added, followed by 6 M hydrochloric acid (84 mL) dropwise at 0° C. The resulting mixture was stirred for 5 h at 100° C. The mixture was allowed to cool and was concentrated under vacuum. The resulting mixture was then diluted with H₂O (100 mL) and basified to pH 8 with 2 M NaOH. The resulting solids were filtered and dried under vacuum to give 2-[6-bromoimidazo[1,5-a]pyridin-1-yl]-1H-imidazole (15.1 g) as a black solid which was used in the next step directly without further purification.

LCMS (ES, m/z): [M+H]⁺: 263

### Synthesis of 2-e: 2-[6-bromoimidazo[1,5-a]pyridin-1-yl]-1-[[2-(trimethylsilyl)ethoxy]methyl]imidazole

To a stirred solution of 2-[6-bromoimidazo[1,5-a]pyridin-1-yl]-1H-imidazole (15 g, 57.3 mmol, 1 equiv) in tetrahydrofuran (150 mL) was added NaH (4.8 g, 200 mmol, 3.5 equiv, 60%) in portions at 0° C. The resulting mixture was stirred for 0.5 h at 0° C. SEM-C1 (15.3 mL, 91 mmol, 1.5 equiv) was added dropwise over 10 min at 0° C. and the resulting mixture was stirred for 1 h at room temperature. The reaction was quenched by the addition of H₂O (500 mL) and was extracted with EA (3×200 mL). The combined organics were washed with brine (2×100 mL), then dried over anhydrous Na₂SO₄. After filtration, the filtrate was concentrated under reduced pressure. The residue was purified by silica gel column chromatography, eluting with PE/EA (1/1) to afford 2-[6-bromoimidazo[1,5-a]pyridin-1-yl]-1-[[2-(trimethylsilyl)ethoxy]methyl]imidazole (8 g, 36% yield) as a brown oil.

LCMS (ES, m/z): [M+H]⁺: 393

### Synthesis of 2-f: 2-[6-(4,4,5,5-tetramethyl-1,3,2-dioxaborolan-2-yl)imidazo[1,5-a]pyridin-1-yl]-1-[[2-(trimethylsilyl)ethoxy]methyl]imidazole

To a stirred solution of 2-[6-bromoimidazo[1,5-a]pyridin-1-yl]-1-[[2-(trimethylsilyl)ethoxy]methyl]imidazole (8 g, 20 mmol, 1 equiv), KOAc (4 g, 41 mmol, 2 equiv) and bis(pinacolato)diboron (5.7 g, 22 mmol, 1.1 equiv) in dioxane (100 mL) was added Pd(dppf)Cl₂ (1.5 g, 2.03 mmol, 0.1 equiv) in portions at room temperature under nitrogen atmosphere. The resulting mixture was stirred for 2 h at 90° C. under nitrogen atmosphere. The mixture was allowed to cool down to room temperature and then diluted with water (500 mL). The resulting mixture was extracted with EA (3×100 mL). The combined organic layers were washed with brine (100 mL), then dried over anhydrous Na₂SO₄. After filtration, the filtrate was concentrated under reduced pressure to give 2-[6-(4,4,5,5-tetramethyl-1,3,2-dioxaborolan-2-yl)imidazo[1,5-a]pyridin-1-yl]-1-[[2-(trimethylsilyl)ethoxy]methyl]imidazole (8.1 g, 90% yield) as a yellow oil which was used in the next step directly without further purification.

LCMS (ES, m/z): [M+H]⁺: 441

### Synthesis of 2-g: 5-chloro-N-[2,4-difluoro-3-[1-(1-[[2-(trimethylsilyl)ethoxy]methyl]imidazol-2-yl)imidazo[1,5-a]pyridin-6-yl]phenyl]-2-methoxypyridine-3-sulfonamide

To a stirred solution of 2-[6-(4,4,5,5-tetramethyl-1,3,2-dioxaborolan-2-yl)imidazo[1,5-a]pyridin-1-yl]-1-[[2-(trimethylsilyl)ethoxy]methyl]imidazole (8.1 g, 18.4 mmol, 1 equiv) and N-(3-bromo-2,4-difluorophenyl)-5-chloro-2-methoxypyridine-3-sulfonamide (9.13 g, 22 mmol, 1.2 equiv) in dioxane (80 mL) and H₂O (8 mL) were added K₂CO₃ (7.6 g, 55 mmol, 3 equiv) and Pd(dppf)Cl₂ (1.35 g, 1.8 mmol, 0.1 equiv) in portions. The resulting mixture was stirred for 3 h at 90° C. under nitrogen atmosphere. The mixture was allowed to cool to room temperature and then diluted with water (200 mL). The resulting mixture was extracted with EA (3×100 mL). The combined organic layers were washed with brine (100 mL), then dried over anhydrous Na₂SO₄. After filtration, the filtrate was concentrated under reduced pressure. The residue was purified by silica gel column chromatography, eluting with PE/EA (1/1) to afford 5-chloro-N-[2,4-difluoro-3-[1-(1-[[2-(trimethylsilyl)ethoxy]methyl]imidazol-2-yl)imidazo[1,5-a]pyridin-6-yl]phenyl]-2-methoxypyridine-3-sulfonamide (5.88 g, 49% yield) as a white solid.

LCMS (ES, m/z): [M+H]⁺: 647

### Synthesis of Compound 2: 5-chloro-N-[2,4-difluoro-3-[1-(1H-imidazol-2-yl)imidazo[1,5-a]pyridin-6-yl]phenyl]-2-methoxypyridine-3-sulfonamide

A solution of 5-chloro-N-[2,4-difluoro-3-[1-(1-[[2-(trimethylsilyl)ethoxy]methyl]imidazol-2-yl)imidazo[1,5-a]pyridin-6-yl]phenyl]-2-methoxypyridine-3-sulfonamide (5.9 g, 9.1 mmol, 1 equiv) in TFA (50 mL) was stirred for 1 h at 70° C. The resulting mixture was concentrated under reduced pressure and then diluted with water (50 mL). The mixture was basified to pH 8 with saturated aqueous NaHCO₃. The resulting mixture was extracted with EA (3×100 mL). The combined organic layers were washed with brine (100 mL), then dried over anhydrous Na₂SO₄. After filtration, the filtrate was concentrated under reduced pressure. The residue was purified by prep-HPLC with the following conditions: Column, welch Vltimate XB-C18, 50×250 mm, 10 μm mobile phase, Mobile Phase A: 0.1% NH₃·H₂O in Water, Mobile Phase B: MeCN (25-65% over 15 min) Detector, 220 nm; to afford 5-chloro-N-[2,4-difluoro-3-[1-(1H-imidazol-2-yl)imidazo[1,5-a]pyridin-6-yl]phenyl]-2-methoxypyridine-3-sulfonamide (2.05 g, 44% yield) as a white solid.

LCMS (ES, m/z): [M+H]⁺: 517

¹H NMR (300 MHz, DMSO-d₆) δ 8.52 (d, J=4.5 Hz, 3H), 8.24 (d, J=9.4 Hz, 1H), 8.09 (d, J=2.7 Hz, 1H), 7.38 (td, J=8.9, 6.0 Hz, 1H), 7.25 (t, J=9.5 Hz, 1H), 7.08 (s, 2H), 6.84 (d, J=8.0 Hz, 1H), 3.93 (s, 3H).

### Example 18: Synthesis of 6-[3-(5-chloro-2-methoxypyridine-3-sulfonamido)-2,6-difluorophenyl]-N-methylimidazo[1,5-a]pyrazine-1-carboxamide (Compound 3

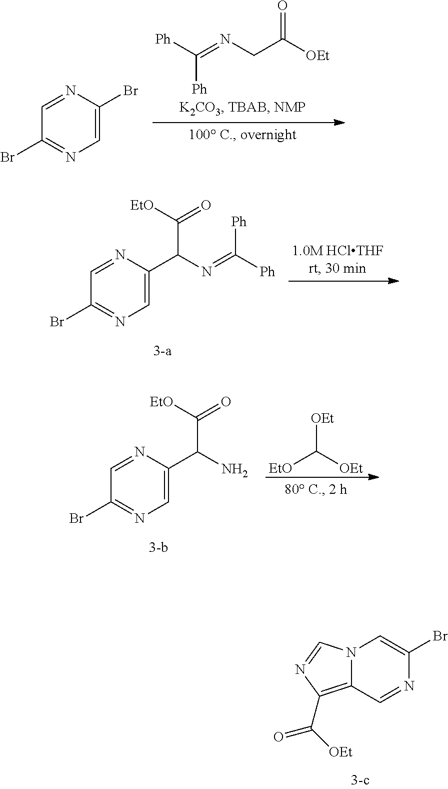 [SMILES: CCOC(=O)CN=C(c1ccccc1)c1ccccc1 ; Brc1cnc(Br)cn1 ; CCOC(=O)C(N=C(c1ccccc1)c1ccccc1)c1cnc(Br)cn1 ; CCOC(=O)C(N)c1cnc(Br)cn1 ; CCOC(OCC)OCC ; CCOC(=O)c1ncn2cc(Br)ncc12]

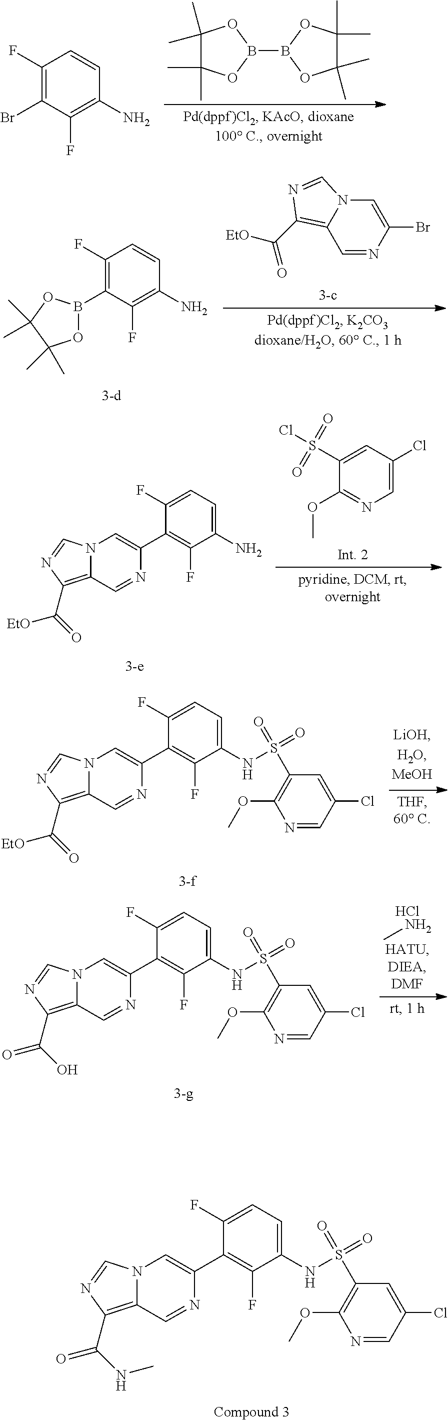

### Synthesis of 3-a: ethyl 2-(5-bromopyrazin-2-yl)-2-[(diphenylmethylidene) amino]acetate

2,5-Dibromopyrazine (10 g, 42 mmol, 1 equiv), ethyl 2-[(diphenylmethylidene)amino]acetate (11.8 g, 44 mmol, 1.05 equiv), TBAB (13.6 g, 42 mmol, 1 equiv) and K₂CO₃ (17.4 g, 126 mmol, 3 equiv) in NMP (200 mL) were stirred overnight at 100° C. in an oil bath. The reaction mixture was cooled and filtered. The filtrate was diluted with 200 mL of water. The resulting solution was extracted with 2×200 mL of ethyl acetate and the organic layers combined. The resulting mixture was washed with 2×200 ml of water. The mixture was dried over anhydrous sodium sulfate and concentrated. The residue was applied to a silica gel column, eluting with ethyl acetate/PE (1/10). The collected fractions were combined and concentrated to give ethyl 2-(5-bromopyrazin-2-yl)-2-[(diphenylmethylidene)amino]acetate (8 g, 45% yield) as a yellow solid.

LCMS (ES, m/z): [M+H]⁺: 424

### Synthesis of 3-b: ethyl 2-amino-2-(5-bromopyrazin-2-yl) acetate

Into a 250 mL round-bottom flask, was placed ethyl 2-(5-bromopyrazin-2-yl)-2-[(diphenylmethylidene)amino]acetate (8 g, 18.8 mmol, 1 equiv), THE (10 mL) and HCl (aqueous, 1 M) (20 mL). The resulting solution was stirred for 30 min at 25° C. The solution formed was diluted with 50 mL of water, and extracted with 2×50 mL of dichloromethane. The aqueous layers were adjusted to pH 8 with NH₃·H₂O and further extracted with 3×50 mL of dichloromethane. The combined organic layers were dried over anhydrous sodium sulfate and concentrated. Ethyl 2-amino-2-(5-bromopyrazin-2-yl)acetate (4.7 g, 96% yield) was isolated as a yellow solid which was used in next step directly without further purification.

LCMS (ES, m/z): [M+H]⁺: 260

### Synthesis of 3-c: ethyl 6-bromoimidazo[1,5-a]pyrazine-1-carboxylate

Into a 50 mL round-bottom flask, was placed ethyl 2-amino-2-(5-bromopyrazin-2-yl) acetate (4.2 g, 0.02 mol, 1 equiv) and triethyl orthoformate (20 mL). The resulting solution was stirred for 2 h at 80° C. in an oil bath. The reaction mixture was cooled, and the solids collected by filtration. Air drying gave ethyl 6-bromoimidazo[1,5-a]pyrazine-1-carboxylate (2.2 g, 50% yield) as a brown solid.

LCMS (ES, m/z): [M+H]⁺: 270

### Synthesis of 3-d: 2,4-difluoro-3-(4,4,5,5-tetramethyl-1,3,2-dioxaborolan-2-yl)aniline

3-Bromo-2,4-difluoroaniline (10 g, 48 mmol, 1 equiv), Pd(dppf)Cl₂ (3.5 g, 4.8 mmol, 0.1 equiv), bis(pinacolato)diboron (18.3 g, 72 mmol, 1.5 equiv) and KOAc (14.2 g, 144.2 mmol, 3 equiv) were dissolved in dioxane (240 mL). The resulting solution was stirred overnight at 100° C. in an oil bath. The reaction mixture was cooled and the solids removed by filtration. The filtrate was concentrated and diluted with DCM (100 mL), then washed with 2×100 mL of water and 100 mL of brine. The organic phase was dried over anhydrous sodium sulfate and concentrated under reduced pressure. The residue was applied to a silica gel column, eluting with ethyl acetate/petroleum ether (1/10). 2,4-Difluoro-3-(4,4,5,5-tetramethyl-1,3,2-dioxaborolan-2-yl)aniline (8 g, 65% yield) was isolated as a yellow solid.

LCMS (ES, m/z): [M+H]⁺: 256

### Synthesis of 3-e: ethyl 6-(3-amino-2,6-difluorophenyl)imidazo[1,5-a]pyrazine-1-carboxylate

Ethyl 6-bromoimidazo[1,5-a]pyrazine-1-carboxylate (500 mg, 1.9 mmol, 1 equiv), 2,4-difluoro-3-(4,4,5,5-tetramethyl-1,3,2-dioxaborolan-2-yl)aniline (708 mg, 2.8 mmol, 1.5 equiv), Pd(dppf)Cl₂ (135 mg, 0.2 mmol, 0.1 equiv), K₂CO₃ (767 mg, 5.6 mmol, 3 equiv) in dioxane (10 mL) and H₂O (2 mL) were stirred for 1 h at 60° C. in an oil bath. The reaction mixture was cooled, diluted with water (20 ml) and extracted with 3×20 mL of dichloromethane. The organic layers were dried over anhydrous sodium sulfate and concentrated. The residue was applied to a silica gel column, and eluted with ethyl acetate/PE (1/2). Ethyl 6-(3-amino-2,6-difluorophenyl)imidazo[1,5-a]pyrazine-1-carboxylate (200 mg 34% yield) was isolated as a brown solid.

LCMS (ES, m/z): [M+H]⁺: 319

### Synthesis of 3-f: ethyl 6-[3-(5-chloro-2-methoxypyridine-3-sulfonamido)-2,6-difluorophenyl]imidazo[1,5-a]pyrazine-1-carboxylate

Ethyl 6-(3-amino-2,6-difluorophenyl)imidazo[1,5-a]pyrazine-1-carboxylate (150 mg, 0.5 mmol, 1 equiv) in DCM (5 mL) was treated with pyridine (186 mg, 2.3 mmol, 5 equiv), then 5-chloro-2-methoxypyridine-3-sulfonyl chloride (137 mg, 0.6 mmol, 1.2 equiv).

The resulting solution was stirred overnight. The resulting mixture was concentrated and purified by Flash-Prep-HPLC with the following conditions: Column, WelFlash™ C18-I, Spherical C18 20-40 m; mobile phase: 0.1% Formic Acid/5-70% MeCN over 15 min; Detector, 254 & 220 nm. Ethyl 6-[3-(5-chloro-2-methoxypyridine-3-sulfonamido)-2,6-difluorophenyl]imidazo[1,5-a]pyrazine-1-carboxylate (320 mg 97% yield) was isolated as a yellow solid.

LCMS (ES, m/z): [M+H]⁺: 524

### Synthesis of 3-g: 6-[3-(5-chloro-2-methoxypyridine-3-sulfonamido)-2,6-difluorophenyl]imidazo[1,5-a]pyrazine-1-carboxylic acid

Ethyl 6-[3-(5-chloro-2-methoxypyridine-3-sulfonamido)-2,6-difluorophenyl]imidazo[1,5-a]pyrazine-1-carboxylate (200 mg, 0.4 mmol, 1 equiv), MeOH (2 mL), THF (2 mL), H₂O (2 mL) and LiOH (27 mg, 1.1 mmol, 3 equiv) were stirred for 1 h at 60° C. in an oil bath. After concentration, the crude product was purified by Flash-Prep-HPLC with the following conditions: Column, WelFlash™ C18-I, Spherical C18 20-40 m; mobile phase: 5-60% MeCN/0.1% ammonia over 15 min; Detector, 254 nm. 6-[3-(5-Chloro-2-methoxypyridine-3-sulfonamido)-2,6-difluorophenyl]imidazo[1,5-a]pyrazine-1-carboxylic acid (170 mg, 90% yield) was isolated as a yellow solid.

LCMS (ES, m/z): [M+H]⁺: 496

### Synthesis of Compound 3: 6-[3-(5-chloro-2-methoxypyridine-3-sulfonamido)-2,6-difluorophenyl]-N-methylimidazo[1,5-a]pyrazine-1-carboxamide

6-[3-(5-Chloro-2-methoxypyridine-3-sulfonamido)-2,6-difluorophenyl]imidazo[1,5-a]pyrazine-1-carboxylic acid (170 mg, 0.3 mmol, 1 equiv) in DMF (4 mL) was treated with DIEA (133 mg, 1 mmol, 3 equiv), methylamine hydrochloride (16 mg, 0.5 mmol, 1.5 equiv) and HATU (195 mg, 0.5 mmol, 1.5 equiv). The resulting solution was stirred for 1 hr, then concentrated. The crude product was purified by Flash-Prep-HPLC with the following conditions: Column, WelFlash™ C18-I, Spherical C18 20-40 m, 120 g; mobile phase: 5-60% MeCN/0.1% formic acid over 20 min. 6-[3-(5-Chloro-2-methoxypyridine-3-sulfonamido)-2,6-difluorophenyl]-N-methylimidazo[1,5-a]pyrazine-1-carboxamide (43 mg, 25% yield) was isolated as an off-white solid.

LCMS (ES, m/z): [M+H]⁺: 509

¹H NMR (300 MHz, DMSO-d₆) δ 10.46 (s, 1H), 9.51 (d, J=1.6 Hz, 1H), 8.66 (d, J=5.0 Hz, 2H), 8.51 (d, J=2.6 Hz, 1H), 8.43 (d, J=4.9 Hz, 1H), 8.09 (d, J=2.6 Hz, 1H), 7.42 (td, J=8.8, 5.8 Hz, 1H), 7.25 (td, J=9.3, 1.4 Hz, 1H), 3.92 (s, 3H), 2.84 (d, J=4.7 Hz, 3H).

### Example 19: Synthesis of 2-[3-(5-Chloro-2-methoxypyridine-3-sulfonamido)-2,6-difluorophenyl]-N-methylimidazo[1,5-b]pyridazine-5-carboxamide (Compound 4)

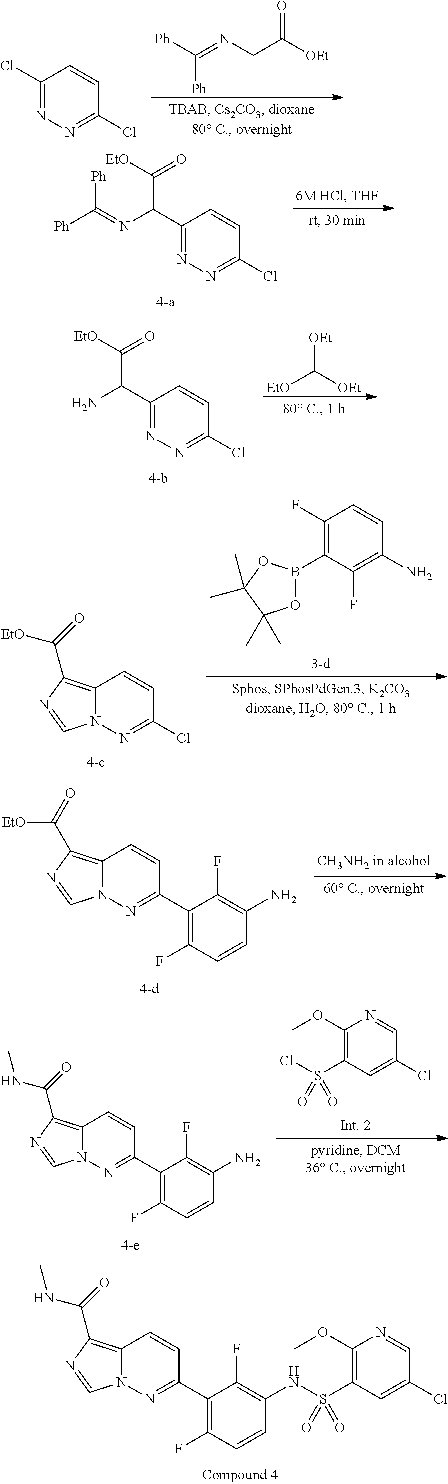

### Synthesis of 4-a: ethyl 2-(6-chloropyridazin-3-yl)-2-[(diphenylmethylidene)amino]acetate

Into a 500 mL round-bottom flask, purged and maintained with an inert atmosphere of nitrogen, was placed 3,6-dichloropyridazine (5 g, 33.5 mmol, 1 equiv), dioxane (160 mL), TBAB (10.8 g, 34 mmol, 1 equiv), Cs₂CO₃ (32.8 g, 100 mmol, 3 equiv) and ethyl 2-[(diphenylmethylidene)amino]acetate (9 g, 34 mmol, 1 equiv). The resulting solution was stirred overnight at 80° C. in an oil bath, then cooled. The resulting mixture was concentrated and the residue applied to a silica gel column, eluting with ethyl acetate/petroleum ether (1:5). The appropriate fractions were combined and concentrated to give ethyl 2-(6-chloropyridazin-3-yl)-2-[(diphenylmethylidene)amino]acetate (6 g, 47% yield) as a brown oil.

LCMS (ES, m/z): [M+H]⁺: 380

### Synthesis of 4-b: ethyl 2-amino-2-(6-chloropyridazin-3-yl)acetate

Into a 250 mL round-bottom flask, was placed ethyl 2-(6-chloropyridazin-3-yl)-2-[(diphenylmethylidene)amino]acetate (6.80 g, 17.9 mmol, 1 equiv), THE (20 mL) and HCl (6 M in water) (20 mL). The resulting solution was stirred for 30 min at 25° C. The resulting solution was diluted with 50 mL of water, and extracted with 2×50 mL of dichloromethane. The aqueous layer was adjusted to pH 8 with NH₃·H₂O and extracted with further 2×50 mL of dichloromethane. The combined organic layers were dried over anhydrous sodium sulfate and concentrated to give ethyl 2-amino-2-(6-chloropyridazin-3-yl)acetate (3.2 g) as a yellow solid, which was used in next step directly without further purification.

LCMS (ES, m/z): [M+H]⁺: 216

### Synthesis of 4-c: ethyl 2-chloroimidazo[1,5-b]pyridazine-5-carboxylate

Into a 250 mL round-bottom flask, was placed ethyl 2-amino-2-(6-chloropyridazin-3-yl)acetate (3.1 g, 14.4 mmol, 1 equiv) and triethoxymethane (50 mL). The resulting solution was stirred for 1 h at 80° C. in an oil bath. After concentration, the residue was applied to a silica gel column, eluting with ethyl acetate/petroleum ether (1/1). The appropriate fractions were combined and concentrated to give ethyl 2-chloroimidazo[1,5-b]pyridazine-5-carboxylate (2.4 g, 74% yield) as a yellow solid.

LCMS (ES, m/z): [M+H]⁺: 226

### Synthesis of 4-d: ethyl 2-(3-amino-2,6-difluorophenyl)imidazo[1,5-b]pyridazine-5-carboxylate

Into a 40 mL vial, was placed ethyl 2-chloroimidazo[1,5-b]pyridazine-5-carboxylate (500 mg, 2.2 mmol, 1 equiv), H₂O (2 mL), dioxane (10 mL), Sphos (182 mg, 0.4 mmol, 0.2 equiv), SPhosPdGen.3 (173 mg, 0.2 mmol, 0.1 equiv), 2,4-difluoro-3-(4,4,5,5-tetramethyl-1,3,2-dioxaborolan-2-yl)aniline (1.1 g, 4.4 mmol, 2 equiv) and K₂CO₃ (766 mg, 5.5 mmol, 2.5 equiv). The resulting solution was stirred for 1 h at 80° C. in an oil bath. The reaction mixture was cooled and concentrated. The residue was applied to a silica gel column, eluting with ethyl acetate/petroleum ether (1/1). The appropriate fractions were combined and concentrated to give ethyl 2-(3-amino-2,6-difluorophenyl)imidazo[1,5-b]pyridazine-5-carboxylate (340 mg, 48% yield) as a yellow solid.

LCMS (ES, m/z): [M+H]⁺: 319

### Synthesis of 4-e: 2-(3-amino-2,6-difluorophenyl)-N-methylimidazo[1,5-b]pyridazine-5-carboxamide

Into a 50 mL round-bottom flask, was placed ethyl 2-(3-amino-2,6-difluorophenyl)imidazo[1,5-b]pyridazine-5-carboxylate (330 mg, 1 mmol, 1 equiv) and methylamine (5 mL, 33% in alcohol). The resulting solution was stirred overnight at 60° C. in an oil bath. The reaction mixture was cooled and concentrated. The crude product was purified by Flash-Prep-HPLC with the following conditions: Column, WelFlash™ C18-I, Spherical C18 20-40 m, 120 g; mobile phase: 5-60% MeCN/0.1% Formic Acid over 20 min; Detector, UV 254 nm. 2-(3-Amino-2,6-difluorophenyl)-N-methylimidazo[1,5-b]pyridazine-5-carboxamide (240 mg, 76% yield) was isolated as a yellow solid.

LCMS (ES, m/z): [M+H]⁺: 304

### Synthesis of Compound 4: 2-[3-(5-Chloro-2-methoxypyridine-3-sulfonamido)-2,6-difluorophenyl]-N-methylimidazo[1,5-b]pyridazine-5-carboxamide

Into an 8 mL vial, was placed 2-(3-amino-2,6-difluorophenyl)-N-methylimidazo[1,5-b]pyridazine-5-carboxamide (70 mg, 0.2 mmol, 1 equiv), DCM (2 mL), pyridine (91 mg, 1.1 mmol, 5 equiv) and 5-chloro-2-methoxypyridine-3-sulfonyl chloride (84 mg, 0.3 mmol, 1.5 equiv). The resulting solution was stirred overnight at 36° C. in an oil bath. The resulting mixture was concentrated. The crude product was purified by Prep-HPLC with the following conditions: Column, welch Vltimate XB-C18, 50×250 mm, 10 μm mobile phase, Mobile Phase A: 0.1% FA in Water, Mobile Phase B: ACN (44% Phase B up to 58% in 8 min); Detector, 220 nm. 2-[3-(5-Chloro-2-methoxypyridine-3-sulfonamido)-2,6-difluorophenyl]-N-methylimidazo[1,5-b]pyridazine-5-carboxamide (69 mg, 59% yield) was isolated as an off-white solid.

LCMS (ES, m/z): [M+H]⁺: 509 [M+H]⁺

¹H NMR (300 MHz, DMSO-d₆) δ 10.53 (s, 1H), 8.87 (s, 1H), 8.58 (d, J=9.5 Hz, 1H), 8.51 (d, J=2.6 Hz, 1H), 8.33 (d, J=4.8 Hz, 1H), 8.10 (d, J=2.6 Hz, 1H), 7.50 (td, J=9.0, 5.9 Hz, 1H), 7.30 (td, J=9.1, 1.6 Hz, 1H), 7.13 (dt, J=9.4, 1.3 Hz, 1H), 3.91 (s, 3H), 2.82 (d, J=4.7 Hz, 3H).

### Example 20: Synthesis of 2-[3-(5-chloro-2-methylpyridine-3-sulfonamido)-2,6-difluorophenyl]-N-methylimidazo[1,5-b]pyridazine-5-carboxamide (Compound 5)

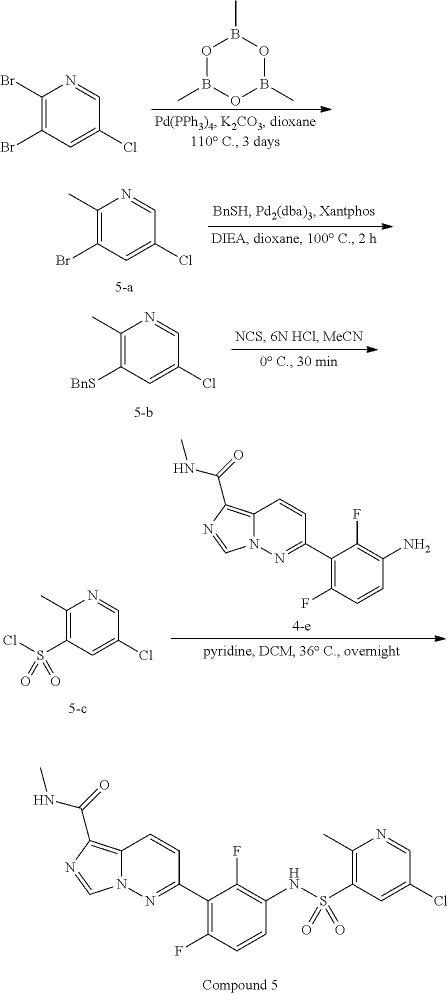

### Synthesis of 5-a: 3-bromo-5-chloro-2-methylpyridine

Into a round-bottom flask purged and maintained with an inert atmosphere of nitrogen, was placed 2,3-dibromo-5-chloropyridine (5 g, 18 mmol, 1 equiv), dioxane (90 mL), K₂CO₃ (7.6 g, 55 mmol, 3 equiv), Pd(PPh₃)₄ (2.1 g, 1.8 mmol, 0.1 equiv) and a 50% yield solution of trimethyl-1,3,5,2,4,6-trioxatriborinane (2.3 g, 18.3 mmol, 1 equiv) in THF.

The resulting solution was stirred at 110° C. in an oil bath for three days, adding a 50% solution of trimethyl-1,3,5,2,4,6-trioxatriborinane (2.3 g, 18.3 mmol, 1 equiv) in THE each day. The reaction mixture was cooled, and the resulting mixture was concentrated at low temperature (the product has a low bp., it can easily be removed with solvent). The residue was applied to a silica gel column, eluting with ethyl acetate/petroleum ether (1:10). 3-Bromo-5-chloro-2-methylpyridine (2 g, 53% yield) was obtained as an off-white solid.

LCMS (ES, m/z): [M+H]⁺: 205

### Synthesis of 5-b: 3-(benzylsulfanyl)-5-chloro-2-methylpyridine

Into a 50 mL round-bottom flask, purged and maintained with an inert atmosphere of nitrogen, was placed 3-bromo-5-chloro-2-methylpyridine (850 mg, 4 mmol, 1 equiv), dioxane (20 mL), DIEA (1.06 g, 8.2 mmol, 2 equiv), Xantphos (476 mg, 0.8 mmol, 0.2 equiv), Pd₂(dba)₃ (377 mg, 0.4 mmol, 0.1 equiv) and benzyl mercaptan (767 mg, 6.1 mmol, 1.5 equiv). The resulting solution was stirred for 2 h at 100° C. in an oil bath. The reaction mixture was cooled, and concentrated. The residue was applied to a silica gel column, eluting with ethyl acetate/petroleum ether (1:20). 3-(Benzylsulfanyl)-5-chloro-2-methylpyridine (500 mg, 49% yield) was isolated as a yellow solid.

LCMS (ES, m/z): [M+H]⁺: 250

### Synthesis of 5-c: 5-chloro-2-methylpyridine-3-sulfonyl chloride

Into a 20 mL vial was placed MeCN (6 mL) and HCl (6 M in water, 1.2 mL). This was followed by the addition of NCS (428 mg, 3.2 mmol, 4 equiv) in portions at 0° C. The mixture was stirred for 10 mins, then 3-(benzylsulfanyl)-5-chloro-2-methylpyridine (200 mg, 0.8 mmol, 1 equiv) was added in portions at 0° C. The resulting solution was stirred for 30 min at 0° C. in a water/ice bath. The reaction was quenched by the addition of 20 mL of water. The resulting solution was extracted with 2×20 mL of dichloromethane and the organic layers combined. The organics were washed with 2×20 mL of water and the mixture dried over anhydrous sodium sulfate before being concentrated. 5-Chloro-2-methylpyridine-3-sulfonyl chloride (300 mg crude) was isolated as a yellow oil, which was used in next step directly without further purification.

### Synthesis of Compound 5: 2-[3-(5-chloro-2-methylpyridine-3-sulfonamido)-2,6-difluorophenyl]-N-methylimidazo[1,5-b]pyridazine-5-carboxamide

Into an 8 mL vial, was placed 2-(3-amino-2,6-difluorophenyl)-N-methylimidazo[1,5-b]pyridazine-5-carboxamide (60 mg, 0.2 mmol, 1 equiv), DCM (2 mL), pyridine (313 mg, 4 mmol, 20 equiv) and 5-chloro-2-methylpyridine-3-sulfonyl chloride (268 mg, 1.2 mmol, 6 equiv). The resulting solution was stirred overnight at 36° C. in an oil bath. The reaction mixture was concentrated, and the crude product was purified by Prep-HPLC with the following conditions: Column, welch Vltimate XB-C18, 50×250 mm, 10 μm mobile phase, 30-60% MeCN over 20 mins/0.1% aqueous formic acid; Detector, UV 254 nm. 2-[3-(5-Chloro-2-methylpyridine-3-sulfonamido)-2,6-difluorophenyl]-N-methylimidazo[1,5-b]pyridazine-5-carboxamide (68 mg, 69% yield) was obtained as an off-white solid.

LCMS (ES, m/z): [M+H]⁺: 493 H NMR (300 MHz, DMSO-d₆) δ 10.87 (s, 1H), 8.86 (s, 1H), 8.78 (d, J=2.4 Hz, 1H), 8.58 (d, J=9.5 Hz, 1H), 8.32 (q, J=4.6 Hz, 1H), 8.08 (d, J=2.4 Hz, 1H), 7.50 (td, J=8.9, 5.8 Hz, 1H), 7.32 (td, J=9.1, 1.6 Hz, 1H), 7.10 (dt, J=9.4, 1.3 Hz, 1H), 2.82 (d, J=4.8 Hz, 3H), 2.77 (s, 3H).

### Example 21: Synthesis of 6-[2,6-difluoro-3-(5-fluoro-2-methylpyridine-3-sulfonamido)phenyl]-N-methylimidazo[1,5-a]pyridine-1-carboxamide (Compound 6)

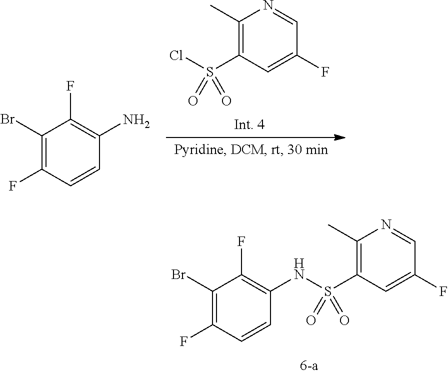 [SMILES: Nc1ccc(F)c(Br)c1F ; Cc1ncc(F)cc1S(=O)(=O)Cl ; Cc1ncc(F)cc1S(=O)(=O)Nc1ccc(F)c(Br)c1F]

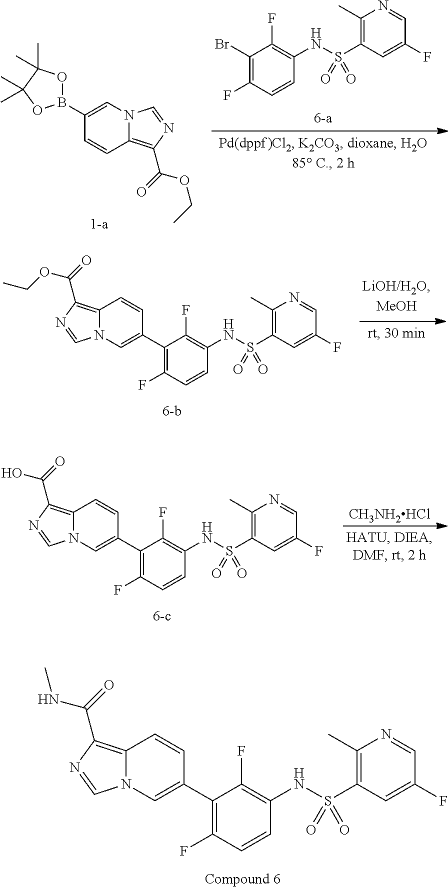 [SMILES: CCOC(=O)c1ncn2cc(B3OC(C)(C)C(C)(C)O3)ccc12 ; Cc1ncc(F)cc1S(=O)(=O)Nc1ccc(F)c(Br)c1F ; CCOC(=O)c1ncn2cc(-c3c(F)ccc(NS(=O)(=O)c4cc(F)cnc4C)c3F)ccc12 ; Cc1ncc(F)cc1S(=O)(=O)Nc1ccc(F)c(-c2ccc3c(C(=O)O)ncn3c2)c1F ; "CH3NH2·HCl" ; CNC(=O)c1ncn2cc(-c3c(F)ccc(NS(=O)(=O)c4cc(F)cnc4C)c3F)ccc12]

### Synthesis of 6-a: N-(3-bromo-2,4-difluorophenyl)-5-fluoro-2-methylpyridine-3-sulfonamide

Into a 25 mL 3-necked round-bottom flask, was placed 3-bromo-2,4-difluoroaniline (596 mg, 2.9 mmol, 1 equiv), DCM (10 mL), pyridine (679 mg, 8.6 mmol, 3 equiv) and 5-fluoro-2-methylpyridine-3-sulfonyl chloride (600 mg, 2.9 mmol, 1 equiv). The resulting solution was stirred for 30 min at room temperature. The mixture was concentrated under reduced pressure, and the residue purified by silica gel column chromatography, eluting with PE:EA (2:1) to afford N-(3-bromo-2,4-difluorophenyl)-5-fluoro-2-methylpyridine-3-sulfonamide (760 mg, 70% yield) as a yellow oil.

LCMS (ES, m/z): [M+H]^{m}: 381

### Synthesis of 6-b: ethyl 6-[2,6-difluoro-3-(5-fluoro-2-methylpyridine-3-sulfonamido)phenyl]imidazo[1,5-a]pyridine-1-carboxylate

Into a 25 mL 3-necked round-bottom flask, was placed ethyl 6-(4,4,5,5-tetramethyl-1,3,2-dioxaborolan-2-yl)imidazo[1,5-a]pyridine-1-carboxylate (200 mg, 0.6 mmol, 1 equiv), dioxane (10 mL), N-(3-bromo-2,4-difluorophenyl)-5-fluoro-2-methylpyridine-3-sulfonamide (241 mg, 0.6 mmol, 1 equiv), K₂CO₃ (262 mg, 1.9 mmol, 3 equiv), H₂O (0.8 mL) and Pd(dppf)Cl₂ (46 mg, 0.06 mmol, 0.1 equiv) in one portion at room temperature under N₂ atmosphere. The resulting solution was stirred for 2 h at 85° C. in an oil bath under N₂ atmosphere. The mixture was allowed to cool to room temperature and was filtered. The filtrate was concentrated under reduced pressure and the residue purified by silica gel column chromatography, eluting with PE:EA (1:1) to afford ethyl-[2-difluoro-3-(5-fluoro-2-methylpyridine-3-sulfonamido)phenyl]imidazo[1,5-a]pyridine-1-carboxylate (200 mg, 64% yield) as a brown solid.

LCMS (ES, m/z): [M+H]⁺: 491

### Synthesis of 6-c: 6-[2,6-difluoro-3-(5-fluoro-2-methylpyridine-3-sulfonamido)phenyl]imidazo[1,5-a]pyridine-1-carboxylic acid

Into a 25 mL round-bottom flask, was placed ethyl 6-[2,6-difluoro-3-(5-fluoro-2-methylpyridine-3-sulfonamido)phenyl]imidazo[1,5-a]pyridine-1-carboxylate (200 mg, 0.4 mmol, 1 equiv), MeOH (10 mL), H₂O (2 mL) and LiOH (30 mg, 1.3 mmol, 3 equiv). The resulting solution was stirred for 1 h at room temperature. The reaction mixture was concentrated under vacuum, and the residue suspended in water (20 mL) and EA (10 mL). The aqueous was extracted with 2×10 mL of ethyl acetate, then the pH was adjusted to 2 with HCl (4 M). This was extracted with 3×10 mL of ethyl acetate and the organic layers were combined. The resulting mixture was washed with 10 mL of brine. The mixture was dried over anhydrous sodium sulfate and concentrated under reduced pressure to give crude 6-[2,6-difluoro-3-(5-fluoro-2-methylpyridine-3-sulfonamido)phenyl]imidazo[1,5-a]pyridine-1-carboxylic acid (153 mg) as a white solid which was used in next step directly without further purification.

LCMS (ES, m/z): [M+H]⁺: 463

### Synthesis of Compound 6: 6-[2,6-difluoro-3-(5-fluoro-2-methylpyridine-3-sulfonamido)phenyl]-N-methylimidazo[1,5-a]pyridine-1-carboxamide

Into a 25 mL round-bottom flask, was placed 6-[2,6-difluoro-3-(5-fluoro-2 methylpyridine-3-sulfonamido)phenyl]imidazo[1,5-a]pyridine-1-carboxylic acid (153 mg, 0.3 mmol, equiv), DMF (5 mL), CH₃NH₂·HCl (22 mg, 0.3 mmol, 1 equiv), HAT U (184 mg, 0.5 mmol, 1.5 equiv) and DIEA (60 mg, 0.5 mmol, 1.5 equiv). The resulting solution was stirred for 2 b at room temperature. The reaction mixture was quenched with H₂O (20 mL) and the aqueous layer extracted with 2×20 mL of dichloromethane. The organics were combined and washed with water (20 mL×3) and brine (20 mL), then dried over anhydrous sodium sulfate. After concentration the residue was purified by prep-HPLC with the following conditions Column, welch Vltimate XB-C18, 50×250 mm, 10 μm mobile phase, Mobile Phase A: 0.05% NH₄HCO₃ in Water, Mobile Phase B: MeCN (15-40% over 15 min) to afford 6-[2,6-difluoro-3-(5-fluoro-2-m-methylpyridine-3-sulfonamido)phenyl]-N-methylimidazo[1,5-a]pyridine-1-carboxamide (22.6 mg, 14% yield) as a white solid.

LCMS (ES, m/z): [M+H]⁺: 476

¹H NMR (300 MHz, DMSO-d₆) δ 10.79 (s, 1H), 8.70 (d, J=2.8 Hz, 1H), 8.58 (s, 1H), 8.48 (s, 1H), 8.18-8.06 (m, 2H), 7.95 (dd, J=8.3, 2.8 Hz, 1H), 7.40-7.27 (m, 1H), 7.20 (t, J=9.3 Hz, 1H), 6.98 (d, J=9.4 Hz, 1H), 2.80 (d, J=4.5 Hz, 3H), 2.77 (d, J=1.2 Hz, 3H).

### Example 22: Synthesis of 6-[2,6-difluoro-3-(5-fluoro-2-methoxypyridine-3-sulfonamido)phenyl]-N-methylimidazo[1,5-a]pyridine-1-carboxamide (Compound 7)

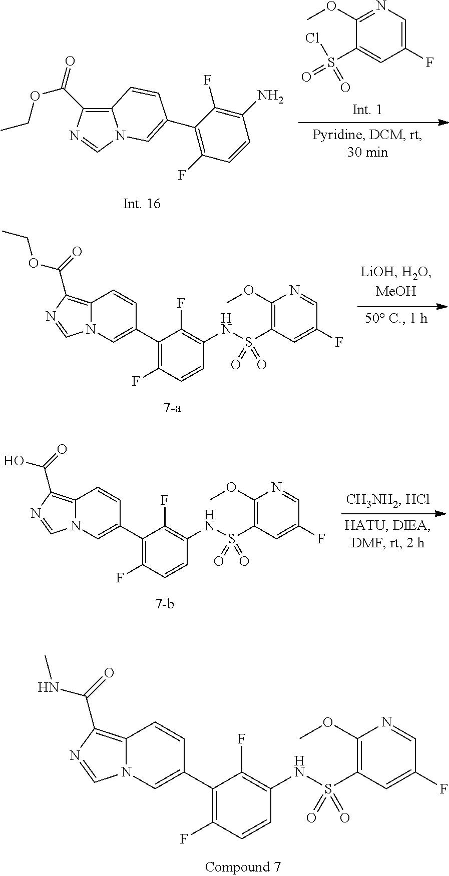

### Synthesis of 7-a: ethyl 6-[2,6-difluoro-3-(5-fluoro-2-methoxypyridine-3-sulfonamido)phenyl]imidazo[1,5-a]pyridine-1-carboxylate

Into a 25 mL round-bottom flask, was placed DCM (5 mL), ethyl 6-(3-amino-2,6-difluorophenyl)imidazo[1,5-a]pyridine-1-carboxylate (150 mg, 0.5 mmol, 1 equiv), pyridine (112 mg, 1.5 mmol, 3 equiv) and 5-fluoro-2-methoxypyridine-3-sulfonyl chloride (120 mg, 0.5 mmol, 1 equiv). The resulting solution was stirred for 30 min at room temperature. The mixture was concentrated under reduced pressure to give crude ethyl 6-[2,6-difluoro-3-(5-fluoro-2-methoxypyridine-3-sulfonamido)phenyl]imidazo[1,5-a]pyridine-1-carboxylate (200 mg) as a yellow oil which was used in next step directly without further purification.

LCMS (ES, m/z): [M+H]⁺: 507

### Synthesis of 7-b: 6-[2,6-difluoro-3-(5-fluoro-2-methoxypyridine-3-sulfonamido)phenyl]imidazo[1,5-a]pyridine-1-carboxylic acid

Into a 50 mL round-bottom flask, was placed MeOH (10 mL), ethyl 6-[2,6-difluoro-3-(5-fluoro-2-methoxypyridine-3-sulfonamido)phenyl]imidazo[1,5-a]pyridine-1-carboxylate (200 mg, 0.4 mmol, 1 equiv), H₂O (2 mL) and LiOH (29 mg, 1.2 mmol, 3 equiv). The resulting solution was stirred for 1 h at room temperature, then concentrated under vacuum. The residue was diluted with water (20 mL) and EA (10 mL). The water layer was extracted with 2×10 mL of ethyl acetate. The aqueous layers were adjusted to pH 2 with HCl (4 M) and then extracted with 3×10 mL of ethyl acetate. The organic layers were combined, washed with 10 mL of brine and dried over anhydrous sodium sulfate. Concentration under reduced pressure gave crude 6-[2,6-difluoro-3-(5-fluoro-2-methoxypyridine-3-sulfonamido)phenyl]imidazo[1,5-a]pyridine-1-carboxylic acid (120 mg) as a light yellow oil which was used in next step directly without further purification.

LCMS (ES, m/z): [M+H]⁺: 479

### Synthesis of Compound 7: 6-[2,6-difluoro-3-(5-fluoro-2-methoxypyridine-3-sulfonamido)phenyl]-N-methylimidazo[1,5-a]pyridine-1-carboxamide

Into a 25 mL round-bottom flask, was placed DMF (5 mL), 6-[2,6-difluoro-3-(5-fluoro-2-methoxypyridine-3-sulfonamido)phenyl]imidazo[1,5-a]pyridine-1-carboxylic acid (120 mg, 0.3 mmol, 1 equiv), CH₃NH₂·HCl (17 mg, 0.3 mmol, 1 equiv), HATU (143 mg, 0.4 mmol, 1.5 equiv) and DIEA (49 mg, 0.4 mmol, 1.5 equiv). The resulting solution was stirred for 2 h at room temperature. The resulting mixture was concentrated and the residue purified by prep-HPLC with the following conditions Column, welch Vltimate XB-C18, 50×250 mm, 10 μm mobile phase, Mobile Phase A: 0.05% NH₄HCO₃ in Water, Mobile Phase B: MeCN (20% to 50% over 15 min) to afford 6-[2,6-difluoro-3-(5-fluoro-2-methoxypyridine-3-sulfonamido)phenyl]-N-methylimidazo[1,5-a]pyridine-1-carboxamide (50 mg, 39% yield) as a white solid.

LCMS (ES, m/z): [M+H]⁺: 491 H NMR (300 MHz, DMSO-d₆) δ 10.45 (s, 1H), 8.60 (s, 1H), 8.52-8.44 (m, 2H), 8.19-8.08 (m, 2H), 8.04 (dd, J=7.3, 3.0 Hz, 1H), 7.37 (td, J=8.9, 5.8 Hz, 1H), 7.31-7.19 (m, 1H), 7.01 (dd, J=9.5, 1.5 Hz, 1H), 3.91 (s, 3H), 2.84-2.77 (m, 3H).

### Example 23: Synthesis of 6-[3-(5-cyano-2-methoxypyridine-3-sulfonamido)-2,6-difluorophenyl]-N-methylimidazo[1,5-a]pyridine-1-carboxamide (Compound 8)

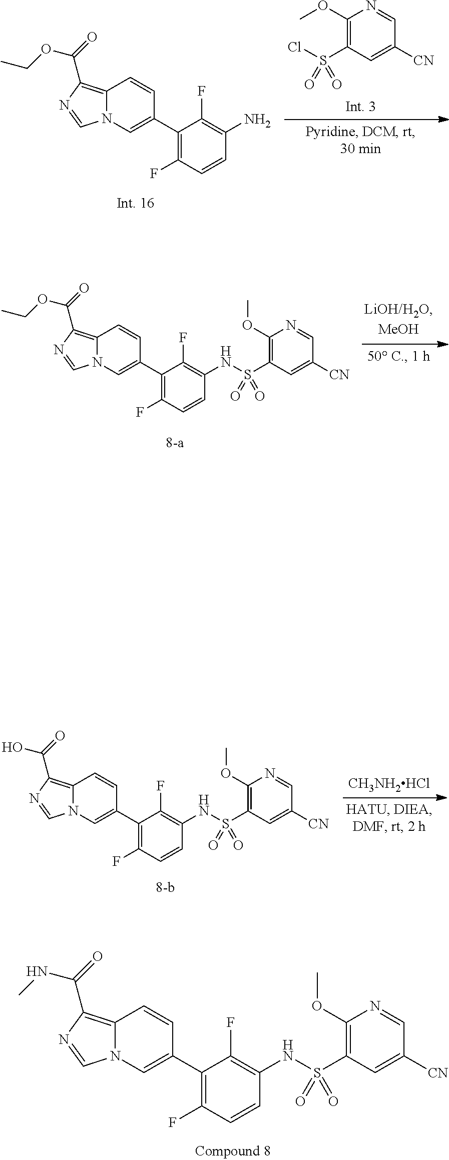 [SMILES: CCOC(=O)c1ncn2cc(-c3c(F)ccc(N)c3F)ccc12 ; COc1ncc(C#N)cc1S(=O)(=O)Cl ; CCOC(=O)c1ncn2cc(-c3c(F)ccc(NS(=O)(=O)c4cc(C#N)cnc4OC)c3F)ccc12 ; COc1ncc(C#N)cc1S(=O)(=O)Nc1ccc(F)c(-c2ccc3c(C(=O)O)ncn3c2)c1F ; "CH3NH2·HCl" ; CNC(=O)c1ncn2cc(-c3c(F)ccc(NS(=O)(=O)c4cc(C#N)cnc4OC)c3F)ccc12]

### Synthesis of 8-a: ethyl 6-[3-(5-cyano-2-methoxypyridine-3-sulfonamido)-2,6-difluorophenyl]imidazo[1,5-a]pyridine-1-carboxylate

Into a 25 mL round-bottom flask, was placed DCM (5 mL), ethyl 6-(3-amino-2,6-difluorophenyl)imidazo[1,5-a]pyridine-1-carboxylate (150 mg, 0.5 mmol, 1 equiv), pyridine (112 mg, 1.4 mmol, 3 equiv) and 5-cyano-2-methoxypyridine-3-sulfonyl chloride (110 mg, 0.5 mmol, 1 equiv). The resulting solution was stirred for 30 min at room temperature. The mixture was concentrated under reduced pressure to give crude ethyl 6-[3-(5-cyano-2-methoxypyridine-3-sulfonamido)-2,6-difluorophenyl]imidazo[1,5-a]pyridine-1-carboxylate (200 mg) as a yellow oil which was used in next step directly without further purification.

LCMS (ES, m/z): [M+H]⁺: 514

### Synthesis of 8-b: 6-[3-(5-cyano-2-methoxypyridine-3-sulfonamido)-2,6-difluorophenyl]imidazo[1,5-a]pyridine-1-carboxylic acid

Into a 50 mL round-bottom flask, was placed MeOH (10 mL), ethyl 6-[3-(5-cyano-2-methoxypyridine-3-sulfonamido)-2,6-difluorophenyl]imidazo[1,5-a]pyridine-1-carboxylate (200 mg, 0.4 mmol, 1 equiv), H₂O (2 mL) and LiOH (28 mg, 1.2 mmol, 3 equiv). The resulting solution was stirred for 1 h at room temperature, then concentrated under vacuum. The residue was diluted with water (20 mL). The water layer was extracted with ethyl acetate (3×10 mL). The aqueous layers were adjusted to pH 2 with HCl (4 M), and then extracted with ethyl acetate (3×10 mL). The organics were combined, washed with brine (10 mL) and dried over anhydrous sodium sulfate. Concentration gave crude 6-[3-(5-cyano-2-methoxypyridine-3-sulfonamido)-2,6-difluorophenyl]imidazo[1,5-a]pyridine-1-carboxylic acid (120 mg) as a yellow oil which was used in next step directly without further purification.

LCMS (ES, m/z): [M+H]⁺: 486

### Synthesis of Compound 8: 6-[3-(5-cyano-2-methoxypyridine-3-sulfonamido)-2,6-difluorophenyl]-N-methylimidazo[1,5-a]pyridine-1-carboxamide

Into a 25 mL round-bottom flask, was placed DMF (5 mL), 6-[3-(5-cyano-2-methoxypyridine-3-sulfonamido)-2,6-difluorophenyl]imidazo[1,5-a]pyridine-1-carboxylic acid (120 mg, 0.3 mmol, 1 equiv), CH₃NH₂·HCl (16 mg, 0.3 mmol, 1 equiv), HATU (141 mg, 0.4 mmol, 1.5 equiv) and DIEA (48 mg, 0.4 mmol, 1.5 equiv). The resulting solution was stirred for 2 h at room temperature, then diluted with H₂O (50 ml). The resulting solution was extracted with 2×50 mL of dichloromethane and the organic layers combined and dried over anhydrous sodium sulfate. After filtration, the filtrate was concentrated under reduced pressure. The residue was purified by prep-HPLC with the following conditions Column, welch Vltimate XB-C18, 50×250 mm, 10 μm mobile phase, Mobile Phase A: 0.05% NH₄HCO₃ in Water, Mobile Phase B: MeCN (20% to 40% over 10 min) to afford 6-[3-(5-cyano-2-methoxypyridine-3-sulfonamido)-2,6-difluorophenyl]-N-methylimidazo[1,5-a]pyridine-1-carboxamide (13 mg, 10%) as a white solid.

LCMS (ES, m/z): [M+H]⁺: 498

¹H NMR (300 MHz, DMSO-d₆) δ 8.83 (s, 1H), 8.58 (s, 1H), 8.47 (s, 1H), 8.41 (s, 1H), 8.17-8.05 (m, 2H), 7.30 (d, J=6.8 Hz, 1H), 7.10 (s, 1H), 7.03 (d, J=9.5 Hz, 1H), 3.96 (s, 3H), 2.80 (d, J=4.7 Hz, 3H).

### Example 24: Synthesis of 6-[3-(5-cyano-2-methylpyridine-3-sulfonamido)-2,6-difluorophenyl]-N-methylimidazo[1,5-a]pyridine-1-carboxamide (Compound 9)

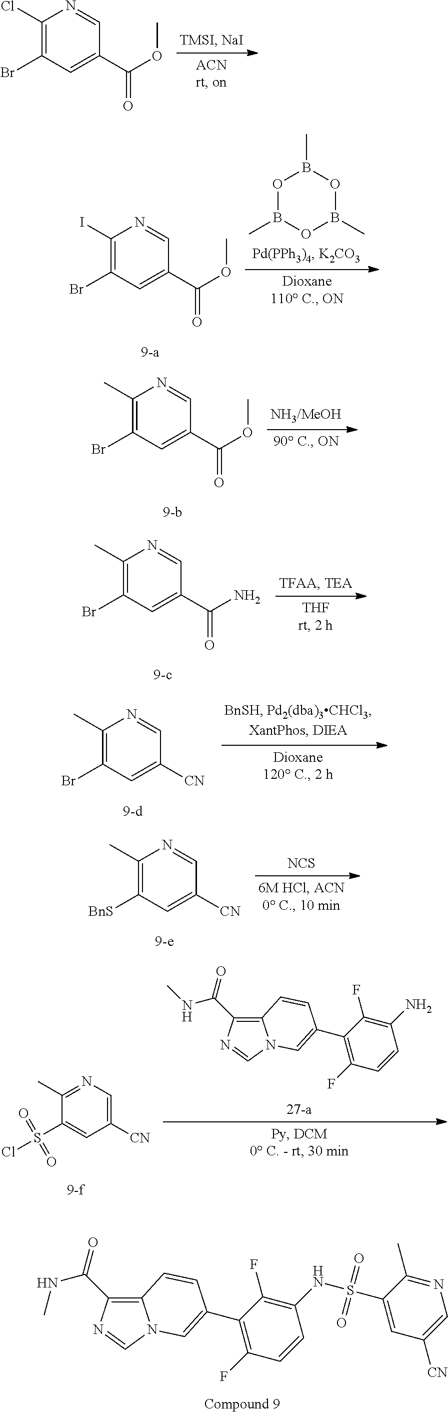

### Synthesis of 9-a: methyl 5-bromo-6-iodopyridine-3-carboxylate

To a stirred solution of methyl 5-bromo-6-chloropyridine-3-carboxylate (40 g, 0.16 mol, 1 equiv) in MeCN (1.2 L) was added TMSI (33 g, 0.17 mol, 1.05 equiv) and NaI (72 g, 0.48 mol, 3 equiv). The mixture was stirred at room temperature overnight, then concentrated. H₂O (0.8 L) was added, and the solution made basic with 2 M aqueous NaOH. The mixture was extracted with DCM (800 mL×3) and the organic layer dried over Na₂SO₄. After concentration, the residue was purified by silica gel column chromatography, eluting with PE:EA (10:1) to afford methyl 5-bromo-6-iodopyridine-3-carboxylate (40 g, 73% yield) as a grey solid.

LCMS (ES, m/z): [M+H]⁺: 342

### Synthesis of 9-b: methyl 5-bromo-6-methylpyridine-3-carboxylate

To a stirred solution of methyl 5-bromo-6-iodopyridine-3-carboxylate (10 g, 29 mmol, 1 equiv) in dioxane (293 mL) was added K₂CO₃ (12 g, 88 mmol, 3 equiv), trimethyl-1,3,5,2,4,6-trioxatriborinane (3.7 g, 29 mmol, 1 equiv) and Pd(PPh₃)₄ (3.4 g, 2.9 mmol, 0.1 equiv). After stirring overnight at 110° C. under a nitrogen atmosphere, LCMS showed 50% desired product and 50% starting material remained, so another batch of trimethyl-1,3,5,2,4,6-trioxatriborinane (3.7 g, 29 mmol, 1 equiv) was added, and the resulting mixture was stirred at 110° C. overnight. After cooling, the resulting mixture was filtered, and the filter cake was washed with EA (100 mL). The filtrate was concentrated under reduced pressure. The residue was purified by silica gel column chromatography, eluting with PE:EA (10:1) to afford methyl 5-bromo-6-methylpyridine-3-carboxylate (5 g, 74% yield) as a white solid.

LCMS (ES, m/z): [M+H]⁺: 230

### Synthesis of 9-c: 5-bromo-6-methylpyridine-3-carboxamide

Methyl-5-bromo-6-methylpyridine-3-carboxylate (3.5 g) was added to 7 M NH₃ in MeOH (70 mL) and stirred at 90° C. in a sealed tube overnight. The reaction solution was concentrated directly to give 5-bromo-6-methylpyridine-3-carboxamide (4 g, crude) as a grey solid.

LCMS (ES, m/z): [M+H]⁺: 215

### Synthesis of 9-d: 5-bromo-6-methylpyridine-3-carbonitrile

To a stirred solution of 5-bromo-6-methylpyridine-3-carboxamide (3 g, 14 mmol, 1 equiv) in THE (60 mL) was added TEA (3.5 g, 35 mmol, 2.5 equiv) and TFAA (6 g, 28 mmol, 2 equiv). The reaction was stirred at room temperature for 2 hours, then concentrated. The residue was purified by silica gel column chromatography, eluting with PE:EA (20:1) to afford 5-bromo-6-methylpyridine-3-carbonitrile (2 g, 74% yield) as a white solid.

LCMS (ES, m/z): [M+H]⁺: 197

### Synthesis of 9-e: 5-(benzylsulfanyl)-6-methylpyridine-3-carbonitrile

To a stirred solution of 5-bromo-6-methylpyridine-3-carbonitrile (2 g, 10 mmol, 1 equiv) in dioxane (26 mL) was added benzyl mercaptan (1.9 g, 15 mmol, 1.5 equiv), DIEA (2.6 g, 20 mmol, 2 equiv), XantPhos (1.2 g, 2 mmol, 0.2 equiv) and Pd₂(dba)₃·CHCl₃ (1 g, 1 mmol, 0.1 equiv) under N₂ atmosphere. The resulting solution was stirred at 120° C. for 2 h, then concentrated directly. The residue was purified by silica gel column chromatography, eluting with PE:EA (10:1) to afford 5-(benzylsulfanyl)-6-methylpyridine-3-carbonitrile (2 g, 82% yield) as a red solid.

LCMS (ES, m/z): [M+H]⁺: 241

### Synthesis of 9-f: 5-cyano-2-methylpyridine-3-sulfonyl chloride

To a stirred solution of 5-(benzylsulfanyl)-6-methylpyridine-3-carbonitrile (0.5 g, 2.1 mmol, 1 equiv) in MeCN (10 mL) was added 6 M HCl (5 mL) dropwise at 0° C. and then NCS (1.1 g, 8.4 mmol, 4 equiv) was added. The reaction was stirred at 0° C. for 10 min. The resulting mixture was diluted with H₂O (10 mL) and the aqueous layer extracted with DCM (20 mL×3). The combined organics were dried over anhydrous Na₂SO₄ and concentrated under reduced pressure. The residue was purified by silica gel column chromatography, eluting with PE:EA (5:1) to afford 5-cyano-2-methylpyridine-3-sulfonyl chloride (0.24 g, 53% yield) as a yellow oil.

### Synthesis of Compound 9: 6-[3-(5-cyano-2-methylpyridine-3-sulfonamido)-2,6-difluorophenyl]-N-methylimidazo[1,5-a]pyridine-1-carboxamide

To a stirred solution of 6-(3-amino-2,6-difluorophenyl)-N-methylimidazo[1,5-a]pyridine-1-carboxamide (180 mg, 0.6 mmol, 1 equiv) in DCM (3 mL) was added 5-cyano-2-methylpyridine-3-sulfonyl chloride (206 mg, 0.95 mmol, 1.6 equiv) and pyridine (141 mg, 1.8 mmol, 3 equiv) at 0° C. The reaction was stirred at room temperature for 30 min, then concentrated. The residue was purified by Prep-HPLC with the following conditions: Column, welch Vltimate XB-C18, 50×250 mm, 10 μm mobile phase, Mobile Phase A: 0.1% aqueous formic acid, Mobile Phase B: MeCN (10% to 50% over 15 min) to give 6-[3-(5-cyano-2-methylpyridine-3-sulfonamido)-2,6-difluorophenyl]-N-methylimidazo[1,5-a]pyridine-1-carboxamide (121 mg, 42% yield) as a white solid.

LCMS (ES, m/z): [M+H]⁺: 483.

¹H NMR (300 MHz, DMSO-d₆) δ 10.88 (s, 1H), 9.14 (d, J=2.0 Hz, 1H), 8.58 (s, 1H), 8.53 (d, J=2.0 Hz, 1H), 8.49 (s, 1H), 8.20-8.05 (m, 2H), 7.42-7.34 (m, 1H), 7.26 (td, J=9.1, 1.5 Hz, 1H), 7.03-6.92 (m, 1H), 2.89 (s, 3H), 2.83-2.79 (m, 3H).

### Example 25: Synthesis of 6-[2,6-difluoro-3-(6-fluoro-1-hydroxy-2,3-dihydro-1H-indene-4-sulfonamido)phenyl]-N-methylimidazo[1,5-a]pyridine-1-carboxamide (Compound 10)

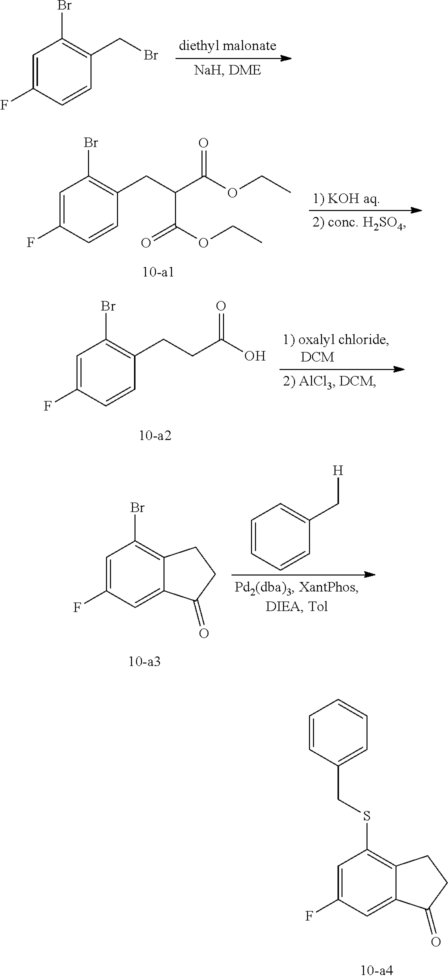

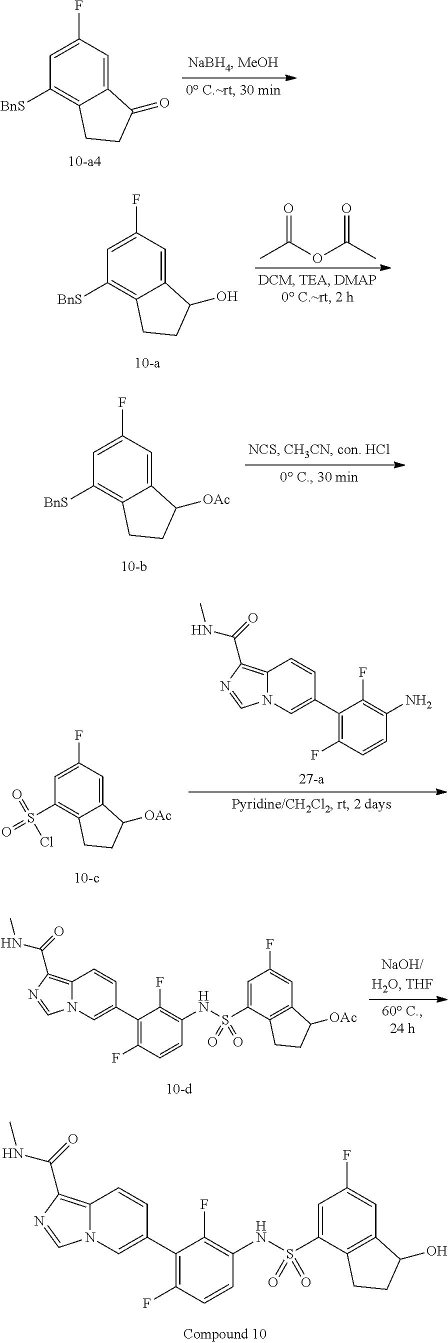

### Synthesis of 10-a1: 1,3-diethyl 2-[(2-bromo-4-fluorophenyl)methyl]propanedioate

Into a 50 mL round-bottom flask was placed NaH (1.1 g, 46 mmol, 2.5 equiv) and 1,2-dimethoxyethane (5 mL). This was followed by the addition of diethyl malonate (4.3 mL) in 1,2-dimethoxyethane (10 mL) dropwise at 0° C. The resulting solution was stirred for 1.5 hr at room temperature. To this was added 2-bromo-1-(bromomethyl)-4-fluorobenzene (5.0 g, 18.7 mmol, 1 equiv) in 1,2-dimethoxyethane (10 mL) dropwise at 0° C. The resulting solution was stirred for 1.5 hr at 85° C. The reaction was quenched with 100 mL of water/ice, and extracted with 3×30 mL of ethyl acetate. The combined organics were dried over anhydrous sodium sulfate and concentrated. The crude product was purified by Flash-Prep-HPLC with the following conditions: Column, silica gel; mobile phase, 0-20% ethyl acetate in petroleum ether; Detector, 254/280 nm. 1,3-Diethyl 2-[(2-bromo-4-fluorophenyl)methyl]propanedioate (6 g, 93% yield) was isolated as a solid.

### Synthesis of 10-a2: 3-(2-bromo-4-fluorophenyl)propanoic acid

Into a 100 mL round-bottom flask was placed 1,3-diethyl 2-[(2-bromo-4-fluorophenyl)methyl]propanedioate (6.0 g, 17 mmol, 1 equiv) and H₂O (40 mL). This was followed by the addition of KOH (2.0 g, 36 mmol, 2.1 equiv). The resulting solution was stirred for 4.5 h at 100° C. then was cooled to 0° C. H₂SO₄ (4.0 mL, 75 mmol, 4.3 equiv) was added and the resulting solution was stirred overnight at 120° C. The solids were removed by filtration and dried to give 3-(2-bromo-4-fluorophenyl)propanoic acid (1.6 g, 37% yield) as a solid.

### Synthesis of 10-a3: 4-bromo-6-fluoro-2,3-dihydroinden-1-one

Into a 100 mL round-bottom flask was placed 3-(2-bromo-4-fluorophenyl)propanoic acid (1.6 g, 6.5 mmol, 1 equiv) in DCM (16 ml). Oxalyl chloride (0.6 ml, 7.2 mmol) was added dropwise at 0° C. and the resulting solution was stirred for 18 hr at RT. The mixture was concentrated. The acid chloride was dissolved in DCM (30 ml), and AlCl₃ (1 g, 1.20 equiv) was added at 0° C., and the resulting solution was stirred for 2 hr at 40° C. The reaction was quenched by the addition of 100 mL of water and extracted with 3×20 mL of dichloromethane. The combined organics were washed with 50 ml of 1 M NaOH, dried over anhydrous sodium sulfate and concentrated. 4-Bromo-6-fluoro-2,3-dihydroinden-1-one (314 mg, 21% yield) was isolated as a solid.

### Synthesis of 10-a4: 4-(benzylsulfanyl)-6-fluoro-2,3-dihydroinden-1-one

Into a 50 mL round-bottom flask purged and maintained with an inert atmosphere of nitrogen was placed 4-bromo-6-fluoro-2,3-dihydroinden-1-one (1.8 g, 7.6 mmol, 1 equiv), benzyl mercaptan (1.1 mL, 1.6 mmol, 1.2 equiv), toluene (20 mL), DIEA (2.5 mL, 15 mmol, 2 equiv) and Xantphos (444 mg, 0.77 mmol, 0.1 equiv). The resulting solution was stirred for 2 hr at 90° C. and then concentrated. The crude product was purified by silica gel chromatography, eluting with 0-10% ethyl acetate/petroleum ether to give 4-(benzylsulfanyl)-6-fluoro-2,3-dihydroinden-1-one (1.1 g, 53% yield) as a solid.

### Synthesis of 10-a: 4-(benzylsulfanyl)-6-fluoro-2,3-dihydro-1H-inden-1-ol

To a stirred solution of 4-(benzylsulfanyl)-6-fluoro-2,3-dihydroinden-1-one (600 mg, 2.2 mmol, 1 equiv) in MeOH (12 mL) was added NaBH₄ (416 mg, 11 mmol, 5 equiv) at 0° C. and the reaction was stirred at room temperature for 30 min. The solution was concentrated and diluted with DCM (5 mL), then washed with H₂O (5 mL×3). The organic layer was dried (MgSO₄) and concentrated, and the residue was purified by silica gel column chromatography, eluting with PE:EA (3:1) to afford 4-(benzylsulfanyl)-6-fluoro-2,3-dihydro-1H-inden-1-ol (0.6 g, 99%) as a yellow solid.

LCMS (ES, m/z): [M+H]⁺: 275

### Synthesis of 10-b: 4-(benzylsulfanyl)-6-fluoro-2,3-dihydro-1H-inden-1-yl acetate

Into a 50 mL 3-necked round-bottom flask, was placed 4-(benzylsulfanyl)-6-fluoro-2,3-dihydro-1H-inden-1-ol (680 mg, 2.5 mmol, 1 equiv), DCM (20 mL), TEA (301 mg, 3.0 mmol, 1.2 equiv) and DMAP (30 mg, 0.25 mmol, 0.1 equiv). This was followed by the addition of acetic anhydride (380 mg, 3.7 mmol, 1.5 equiv) dropwise with stirring at 0° C. The resulting solution was stirred for 2 h at 25° C. The reaction was then quenched by the addition of 50 mL of water. The resulting solution was extracted with 3×30 mL of dichloromethane and the combined organics were washed with 50 ml of brine. The mixture was dried over anhydrous sodium sulfate and concentrated under vacuum. The residue was purified by silica gel column chromatography, eluting with PE:EA (3:1) to afford 4-(benzylsulfanyl)-6-fluoro-2,3-dihydro-1H-inden-1-yl acetate (580 mg, 74% yield) as a white solid.

LCMS (ES, m/z): [M+H]⁺: 317

### Synthesis of 10-c: 4-(chlorosulfonyl)-6-fluoro-2,3-dihydro-1H-inden-1-yl acetate

Into a 50 mL 3-necked round-bottom flask, was placed 4-(benzylsulfanyl)-6-fluoro-2,3-dihydro-1H-inden-1-yl acetate (520 mg, 1.6 mmol, 1 equiv), CH₃CN (10 mL) and HCl (6 mol/L, 5 mL). This was followed by the addition of NCS (878 mg, 6.6 mmol, 4 equiv) in portions at 0° C. The resulting solution was stirred for 30 min at 0° C. The reaction was quenched by the addition of 50 mL of water and extracted with 3×30 mL of ethyl acetate. The combined organics were washed with 50 ml of water and 50 mL of brine, then dried over anhydrous sodium sulfate. Concentration gave crude 4-(chlorosulfonyl)-6-fluoro-2,3-dihydro-1H-inden-1-yl acetate (450 mg) which was used in next step directly without further purification.

LCMS (ES, m/z): [M+H]⁺: 293

### Synthesis of 10-d: 6-[2,6-difluoro-3-(6-fluoro-1-hydroxy-2,3-dihydro-1H-indene-4-sulfonamido)phenyl]-N-methylimidazo[1,5-a]pyridine-1-carboxamide

Into a 40 mL vial, was placed 4-(chlorosulfonyl)-6-fluoro-2,3-dihydro-1H-inden-1-yl acetate (116 mg, 0.4 mmol, 1.2 equiv), DCM (10 mL), pyridine (79 mg, 0.1 mmol, 3 equiv) and 6-(3-amino-2,6-difluorophenyl)-N-methylimidazo[1,5-a]pyridine-1-carboxamide (100 mg, 0.3 mmol, 1 equiv). The resulting solution was stirred for 2 days at 25° C. The mixture was concentrated under vacuum and purified by Prep-HPLC with the following conditions: Column, WelFlash™ C18-I, Spherical C18 20-40 m, 120 g; Mobile Phase A: 0.1% aqueous formic acid, Mobile Phase B: MeCN (10-80% over 15 min) to give 6-[2,6-difluoro-3-(6-fluoro-1-hydroxy-2,3-dihydro-1H-indene-4-sulfonamido)phenyl]-N-methylimidazo[1,5-a]pyridine-1-carboxamide (103 mg, 62% yield) as a white solid.

LCMS (ES, m/z): [M+H]⁺: 559

### Synthesis of Compound 10: 6-[2,6-difluoro-3-(6-fluoro-1-hydroxy-2,3-dihydro-1H-indene-4-sulfonamido)phenyl]-N-methylimidazo[1,5-a]pyridine-1-carboxamide

Into a 40 mL vial, was placed 6-[2,6-difluoro-3-(6-fluoro-1-hydroxy-2,3-dihydro-1H-indene-4-sulfonamido)phenyl]-N-methylimidazo[1,5-a]pyridine-1-carboxamide (100 mg, 0.18 mmol, 1 equiv), THE (10 mL), H₂O (1 mL) and NaOH (14 mg, 0.36 mmol, 2 equiv). The resulting solution was stirred for 24 h at 60° C. The reaction mixture was cooled and concentrated under vacuum and the pH of the solution was adjusted to 7 with HCl. After further concentration, the crude product was purified by Prep-HPLC with the following conditions: Column, welch Vltimate XB-C18, 50×250 mm, 10 μm mobile phase, Mobile Phase A: 0.1% aqueous formic acid, Mobile Phase B: MeCN (10-15% over 15 min) to give 6-[2,6-difluoro-3-(6-fluoro-1-hydroxy-2,3-dihydro-1H-indene-4-sulfonamido)phenyl]-N-methylimidazo[1,5-a]pyridine-1-carboxamide (35 mg, 38% yield) as a white solid.

LCMS (ES, m/z): [M+H]⁺: 517

¹H NMR (300 MHz, DMSO-d₆) δ 10.40 (s, 1H), 8.54 (s, 1H), 8.47 (d, J=0.6 Hz, 1H), 8.18-8.04 (m, 2H), 7.48-7.18 (m, 4H), 6.96 (dd, J=9.4, 1.5 Hz, 1H), 5.60 (d, J=5.6 Hz, 1H), 5.12-5.00 (m, 1H), 3.13-2.99 (m, 1H), 2.84-2.72 (m, 3H), 2.44-2.27 (m, 1H), 1.84-1.66 (m, 1H).

### Example 26: Synthesis of 6-[3-(5-chloro-2-methoxypyridine-3-sulfonamido)-2,6-difluorophenyl]-N,5-dimethyl imidazo[1,5-a]pyridine-1-carboxamide (Compound 11)

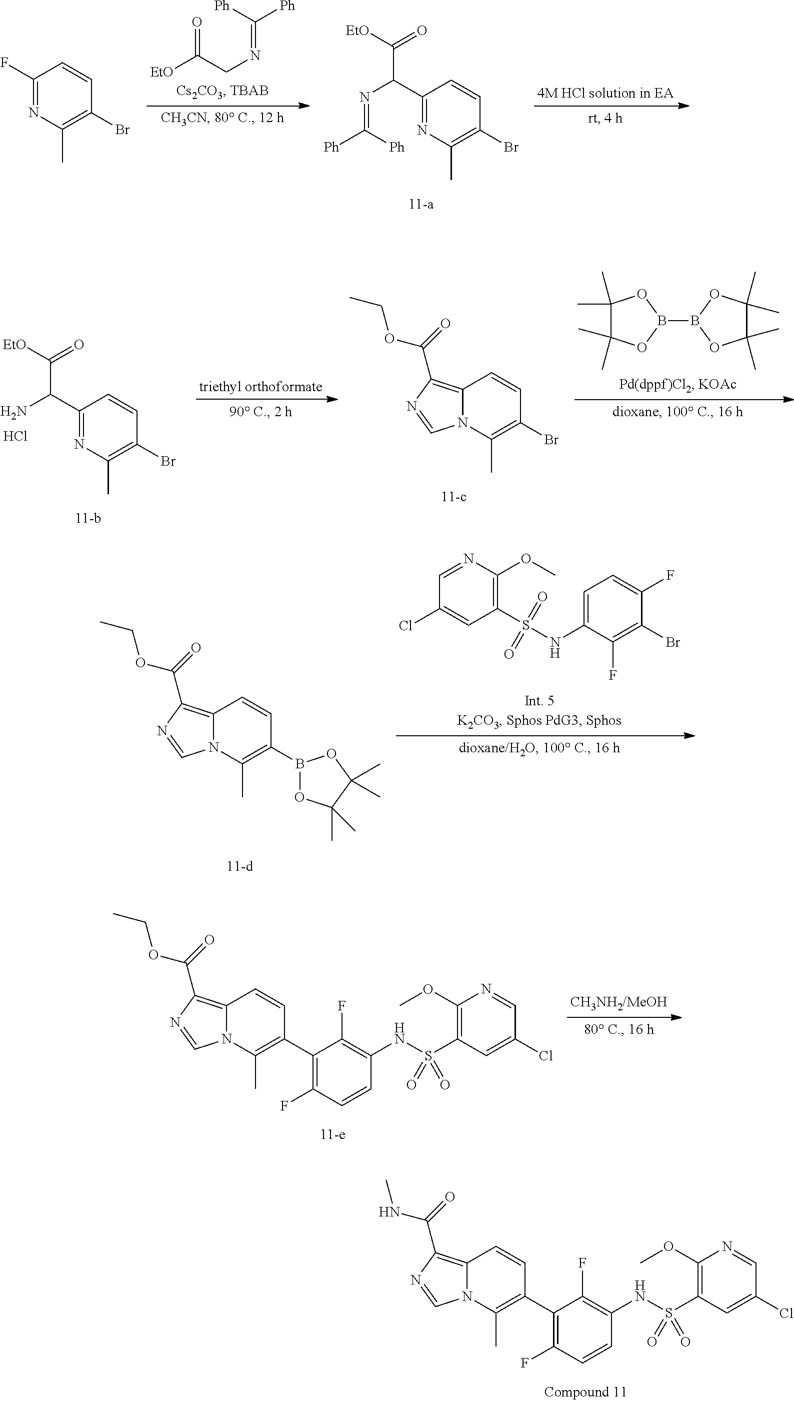

### Synthesis of 11-a: ethyl 2-(5-bromo-6-methylpyridin-2-yl)-2-[(diphenylmethylidene)amino]acetate

To a solution of 3-bromo-6-fluoro-2-methylpyridine (5 g, 26 mmol, 1 equiv) in MeCN (100 mL) were added ethyl 2-[(diphenylmethylidene)amino]acetate (8.44 g, 31.2 mmol, 1.2 equiv), Cs₂CO₃ (12.86 g, 40 mmol, 1.5 equiv) and TBAB (4.24 g, 13 mmol, 0.5 equiv) at room temperature. The resulting mixture was stirred at 80° C. for 12 h. After cooling to room temperature, water (500 mL) was added and the mixture extracted with ethyl acetate (3×200 mL). The combined organics were washed with brine (2×50 mL), dried over anhydrous Na₂SO₄ and filtered. The filtrate was concentrated under reduced pressure to give the crude product, which was purified by silica gel chromatography (eluent: PE:EA 8:1) to afford ethyl 2-(5-bromo-6-methylpyridin-2-yl)-2-[(diphenylmethylidene)amino]acetate (3.2 g, 28% yield) as a yellow solid.

LCMS (ES, m/z): [M+H]⁺: 437

### Synthesis of 11-b: ethyl 2-amino-2-(5-bromo-6-methylpyridin-2-yl)acetate hydrochloride

A solution of ethyl 2-(5-bromo-6-methylpyridin-2-yl)-2-[(diphenylmethylidene)amino]acetate (3.1 g, 7 mmol, 1 equiv) in 4 M HCl solution in ethyl acetate (50 mL) was stirred at room temperature for 4 h. The resulting solids were collected by filtration and dried to afford ethyl 2-amino-2-(5-bromo-6-methylpyridin-2-yl)acetate hydrochloride (1.6 g, 73%) as a white solid.

LCMS (ES, m/z): [M+H]⁺: 273

### Synthesis of 11-c: ethyl 6-bromo-5-methylimidazo[1,5-a]pyridine-1-carboxylate

A solution of ethyl 2-amino-2-(5-bromo-6-methylpyridin-2-yl)acetate (1 g, 3.6 mmol, 1 equiv) in triethyl orthoformate (10 mL) was stirred at 90° C. for 2 h. After cooling to room temperature, water (100 mL) was added and the mixture extracted with ethyl acetate (3×50 mL). The combined organics were washed with brine (2×20 mL) and dried over anhydrous Na₂SO₄. After filtration, the filtrate was concentrated under reduced pressure to give the crude product, which was purified by silica gel chromatography (eluent: PE:EA 1:1) to afford ethyl 6-bromo-5-methylimidazo[1,5-a]pyridine-1-carboxylate (400 mg, 39% yield) as a yellow solid.

LCMS (ES, m/z): [M+H]⁺: 283

### Synthesis of 11-d: ethyl 5-methyl-6-(4,4,5,5-tetramethyl-1,3,2-dioxaborolan-2-yl)imidazo[1,5-a]pyridine-1-carboxylate

To a solution of ethyl 6-bromo-5-methylimidazo[1,5-a]pyridine-1-carboxylate (500 mg, 1.7 mmol, 1 equiv) in dioxane (20 mL) were added bis(pinacolato)diboron (896 mg, 3.5 mmol, 2 equiv), KOAc (346 mg, 3.5 mmol, 2 equiv) and Pd(dppf)Cl₂ (129 mg, 0.17 mmol, 0.1 equiv) at room temperature under nitrogen atmosphere. The resulting mixture was stirred at 100° C. under nitrogen atmosphere for 16 h. After cooling to room temperature, water (100 mL) was added and the mixture extracted with ethyl acetate (3×50 mL). The combined organics were washed with brine (2×50 mL) and dried over anhydrous Na₂SO₄. After filtration, the filtrate was concentrated under reduced pressure to afford ethyl 5-methyl-6-(4,4,5,5-tetramethyl-1,3,2-dioxaborolan-2-yl)imidazo[1,5-a]pyridine-1-carboxylate (400 mg, crude) as a brown solid which was used in the next step without further purification.

LCMS (ES, m/z): [M+H]⁺: 331

### Synthesis of 11-e: ethyl 6-[3-(5-chloro-2-methoxypyridine-3-sulfonamido)-2,6-difluorophenyl]-5-[1,5-a]pyridine-1-carboxylate

To a stirred solution of ethyl 5-methyl-6-(4,4,5,5-tetramethyl-1,3,2-dioxaborolan-2-yl)imidazo[1,5-a]pyridine-1-carboxylate (300 mg, 0.9 mmol, 1 equiv) in dioxane/H₂O (5:1, 12 mL) were added N-(3-bromo-2,4-difluorophenyl)-5-chloro-2-methoxypyridine-3-sulfonamide (450 mg, 1 mmol, 1.2 equiv), K₂CO₃ (314 mg, 2.2 mmol, 2.5 equiv) and SPhos (74 mg, 182 μmol, 0.2 equiv) followed by addition of SPhos Pd G3 (70 mg, 91 μmol, 0.1 equiv) at room temperature under nitrogen atmosphere. The reaction mixture was stirred at 100° C. under nitrogen atmosphere for 16 h. After the mixture was cooled to room temperature, water (100 mL) was added and the mixture extracted with ethyl acetate (3×50 mL). The combined organic layers were washed with brine (2×50 mL) and dried over anhydrous Na₂SO₄. After filtration, the filtrate was concentrated under reduced pressure to give the crude product, which was purified by silica gel chromatography (eluent: PE:EA 1:1) to afford ethyl 6-[3-(5-chloro-2-methoxypyridine-3-sulfonamido)-2,6-difluorophenyl]-5-methylimidazo[1,5-a]pyridine-1-carboxylate (200 mg, 41% yield) as a brown solid.

LCMS (ES, m/z): [M+H]⁺: 537

### Synthesis of Compound 11: 6-[3-(5-chloro-2-methoxypyridine-3-sulfonamido)-2,6-difluorophenyl]-N,5-dimethyl imidazo[1,5-a]pyridine-1-carboxamide

To a stirred solution of CH₃NH₂ in MeOH (30%, 5 mL) was added ethyl 6-[3-(5-chloro-2-methoxypyridine-3-sulfonamido)-2,6-difluorophenyl]-5-methylimidazo[1,5-a]pyridine-1-carboxylate (60 mg, 112 μmol, 1 equiv) at room temperature. The resulting solution was stirred at 80° C. for 16 h. The mixture was concentrated under reduced pressure to give a residue, which was purified by prep-HPLC with the following conditions: Column, welch Vltimate XB-C18, 50×250 mm, 10 μm mobile phase, Mobile Phase A: 0.1% aqueous formic acid, Mobile Phase B: MeCN (30%-60% over 15 min) Detector, 220 nm; to afford 6-[3-(5-chloro-2-methoxypyridine-3-sulfonamido)-2,6-difluorophenyl]-N,5-dimethylimidazo[1,5-a]pyridine-1-carboxamide (50 mg, 64% yield) as a white solid.

LCMS (ES, m/z): [M+H]⁺: 522 H NMR (300 MHz, DMSO-d₆) δ 10.45 (s, 1H), 8.50 (d, J=2.3 Hz, 2H), 8.21-7.99 (m, 3H), 7.52-7.37 (m, 1H), 7.26 (t, J=8.9 Hz, 1H), 6.93 (d, J=9.3 Hz, 1H), 3.94 (s, 3H), 2.84-2.78 (m, 3H), 2.28 (s, 3H).

### Example 27: Synthesis of 2-[2,6-difluoro-3-(5-fluoro-2-methylpyridine-3-sulfonamido)phenyl]-N-methylimidazo[1,5-b]pyridazine-5-carboxamide (Compound 12)

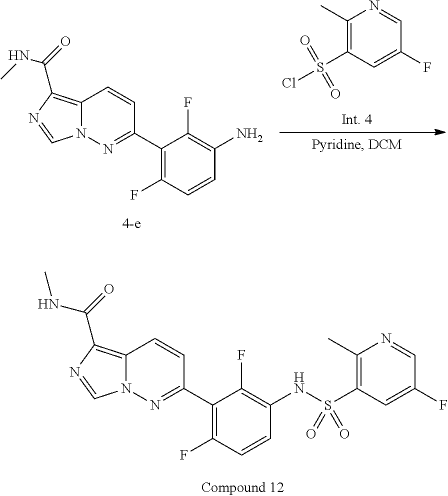 [SMILES: CNC(=O)c1ncn2nc(-c3c(F)ccc(N)c3F)ccc12 ; Cc1ncc(F)cc1S(=O)(=O)Cl ; CNC(=O)c1ncn2nc(-c3c(F)ccc(NS(=O)(=O)c4cc(F)cnc4C)c3F)ccc12]

### Synthesis of Compound 12: 2-[2,6-difluoro-3-(5-fluoro-2-methylpyridine-3-sulfonamido)phenyl]-N-methylimidazo[1,5-b]pyridazine-5-carboxamide

To a stirred mixture of 2-(3-amino-2,6-difluorophenyl)-N-methylimidazo[1,5-b]pyridazine-5-carboxamide (90 mg, 0.3 mmol, 1 equiv) and pyridine (140 mg, 1.8 mmol, 6 equiv) in DCM (3 mL) was added a solution of 5-fluoro-2-methylpyridine-3-sulfonyl chloride (186 mg, 0.9 mmol, 3 equiv) in DCM (1 mL) dropwise at 0° C. The mixture was stirred overnight at room temperature, then concentrated under vacuum. The residue was purified by prep-HPLC with the following conditions: Column, welch Vltimate XB-C18, 50×250 mm, 10 μm mobile phase, Mobile Phase A: 0.1% aqueous formic acid, Mobile Phase B: MeCN (23-43% over 10 min) to afford 2-[2,6-difluoro-3-(5-fluoro-2-methylpyridine-3-sulfonamido)phenyl]-N-methylimidazo[1,5-b]pyridazine-5-carboxamide (40 mg, 28% yield) as a light yellow solid.

LCMS (ES, m/z): [M+H]⁺: 477

¹H NMR (300 MHz, DMSO-d₆) δ 10.86 (s, 1H), 8.86 (s, 1H), 8.74 (d, J=2.8 Hz, 1H), 8.58 (d, J=9.4 Hz, 1H), 8.31 (d, J=5.0 Hz, 1H), 7.96 (dd, J=8.2, 2.9 Hz, 1H), 7.56-7.42 (m, 1H), 7.31 (t, J=8.7 Hz, 1H), 7.10 (d, J=9.4 Hz, 1H), 2.86-2.81 (m, 3H), 2.78 (d, J=1.2 Hz, 3H).

### Example 28: Synthesis of 6-[2,6-difluoro-3-[2-methoxy-5-(trifluoromethyl)pyridine-3-sulfonamido]phenyl]-N-methylimidazo[1,5-a]pyridine-1-carboxamide (Compound 13)

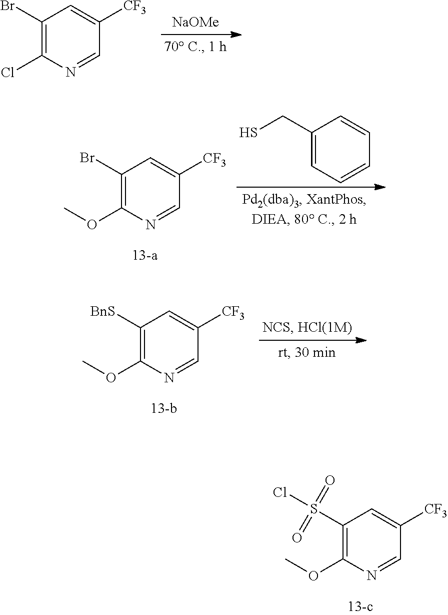

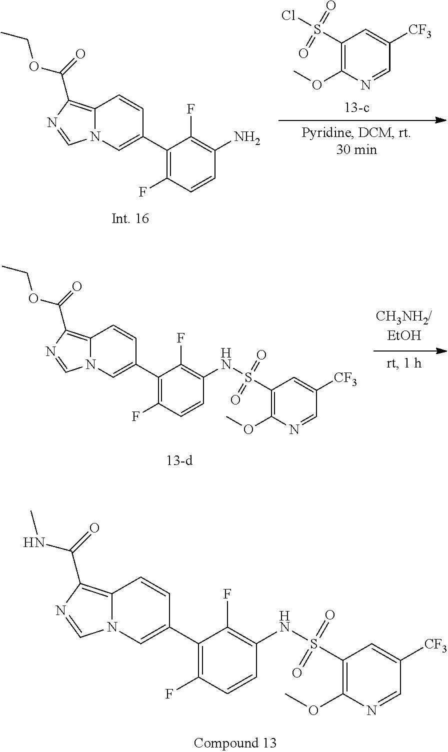 [SMILES: CCOC(=O)c1ncn2cc(-c3c(F)ccc(N)c3F)ccc12 ; COc1ncc(C(F)(F)F)cc1S(=O)(=O)Cl ; CCOC(=O)c1ncn2cc(-c3c(F)ccc(NS(=O)(=O)c4cc(C(F)(F)F)cnc4OC)c3F)ccc12 ; CNC(=O)c1ncn2cc(-c3c(F)ccc(NS(=O)(=O)c4cc(C(F)(F)F)cnc4OC)c3F)ccc12]

### Synthesis of 13-a: 3-bromo-2-methoxy-5-(trifluoromethyl)pyridine

Into a 25-mL round-bottom flask, was placed 25% NaOMe in MeOH (10 mL), 3-bromo-2-chloro-5-(trifluoromethyl)pyridine (3 g, 11 mmol, 1 equiv). The resulting solution was stirred for 1 h at 70° C. in an oil bath. The resulting solution was diluted with 10 mL of H₂O and extracted with 2×10 mL of dichloromethane. After drying over anhydrous sodium sulfate, the solution was concentrated under reduced pressure to give crude 3-bromo-2-methoxy-5-(trifluoromethyl)pyridine (1.6 g) as yellow oil which was used in next step directly without further purification.

LCMS (ES, m/z): [M+H]⁺: 256

### Synthesis of 130-b: 3-(benzylsulfanyl)-2-methoxy-5-(trifluoromethyl)pyridine

Into a 50 mL round-bottom flask, was placed toluene (32 mL), 3-bromo-2-methoxy-5-(trifluoromethyl)pyridine (1.6 g, 6.3 mmol, 1 equiv), benzyl mercaptan (776 mg, 6.3 mmol, 1 equiv), Pd₂(dba)₃ (286 mg, 0.3 mmol, 0.05 equiv), XantPhos (253 mg, 0.4 mmol, 0.07 equiv) and DIEA (2.4 g, 18.8 mmol, 3 equiv). The resulting solution was stirred for 2 h at 80° C. in an oil bath. The reaction mixture was concentrated under vacuum, and the residue purified by silica gel column chromatography, eluting with PE:EA (10:1) to afford 3-(benzylsulfanyl)-2-methoxy-5-(trifluoromethyl)pyridine (1.5 g, 80% yield) as a white solid.

LCMS (ES, m/z): [M+H]⁺: 300

### Synthesis of 13-c: 2-methoxy-5-(trifluoromethyl)pyridine-3-sulfonyl chloride

Into a 50 mL round-bottom flask, was placed CH₃CN (30 mL), NCS (2.6 g, 20 mmol, 3 equiv), HCl (1 M) (7 mL) and 3-(benzylsulfanyl)-2-methoxy-5-(trifluoromethyl)pyridine (2 g, 6.7 mmol, 1 equiv). The resulting solution was stirred for 30 min at room temperature, then concentrated under vacuum. The resulting mixture was diluted with 25 mL of H₂O and extracted with 2×25 mL of ethyl acetate. The combined organics were washed with 20 mL of brine and dried over anhydrous sodium sulfate. Concentration under reduced pressure gave crude 2-methoxy-5-(trifluoromethyl)pyridine-3-sulfonyl chloride (1.4 g) as colorless oil which was used in next step directly without further purification.

### Synthesis of 13-d: ethyl 6-[2,6-difluoro-3-[2-methoxy-5-(trifluoromethyl)pyridine-3-sulfonamido]phenyl]imidazo[1,5-a]pyridine-1-carboxylate

Into a 25-rnL round-bottom flask, was placed ethyl 6-(3-amino-2,6-difluorophenyl)imidazo[1,5-a]pyridine-1-carboxylate (450 mg, 1.4 mmol, 1 equiv), DCM (15 mL), Pyridine (336 mg, 4.3 mmol, 3 equiv) and 2-methoxy-5-(trifluoromethyl)pyridine-3-sulfonyl chloride (391 mg, 1.4 mmol, 1 equiv) The resulting solution was stirred for 30 min at room temperature, then concentrated under vacuum. The residue was diluted with 20 mL of EA and washed with 2×20 mL of H₂O, then 20 mL of brine. After drying over anhydrous sodium sulfate the solution was concentrated under reduced pressure to give crude ethyl 6-[2,6-difluoro-3-[2-methoxy-5-(trifluoromethyl)pyridine-3-sulfonamido]phenyl]imidazo[1,5a]pyridine-1-carboxylate (600 mg) as a colorless oil which was used in next step directly without further purification.

LCMS (ES, m/z): [M+H]^{m}: 557

### Synthesis of Compound 13: 6-[2,6-difluoro-3-[2-methoxy-5-(trifluoromethyl)pyridine-3-sulfonamido]phenyl]-N-methylimidazo[1,5-a]pyridine-1-carboxamide

Into a 25-mL round-bottom flask, was placed ethyl 6-[2,6-difluoro-3-[2-methoxy-5-(trifluoromethyl)pyridine-3-sulfonamido]phenyl]imidazo[1,5-a]pyridine-1-carboxylate (600 mg, 1.1 mmol, 1 equiv) and 33% CH₃NH₂/EtOH (10 mL). The resulting solution was stirred for 1 h at room temperature. The reaction mixture was concentrated under reduced pressure and the residue was purified by Prep-HPLC with the following conditions: Column: welch Vltimate XB-C18, 50×250 mm, 10 μm mobile phase, Mobile Phase A: 0.1% aqueous formic acid, Mobile Phase B: MeCN (20-50% over 20 min) to afford 6-[2,6-difluoro-3-[2-methoxy-5-(trifluoromethyl)pyridine-3-sulfonamido]phenyl]-N-methylimidazo[1,5-a]pyridine-1-carboxamide (39 mg, 7% yield) as a white solid.

LCMS (ES, m/z): [M+H]⁺: 542

¹H NMR (300 MHz, DMSO-d₆) δ 10.56 (s, 1H), 8.88 (s, 1H), 8.60 (s, 1H), 8.49 (s, 1H), 8.27 (d, J=2.4 Hz, 1H), 8.19-8.07 (m, 2H), 7.44-7.33 (m, 1H), 7.27 (d, J=8.9 Hz, 1H), 6.99 (d, J=9.4 Hz, 1H), 3.99 (s, 3H), 2.84-2.77 (m, 3H)

### Example 29: Synthesis of 6-[3-(5-chloro-2-methoxypyridine-3-sulfonamido)-2,6-difluorophenyl]-5-fluoro-N-methylimidazo[1,5-a]pyridine-1-carboxamide (Compound 14)

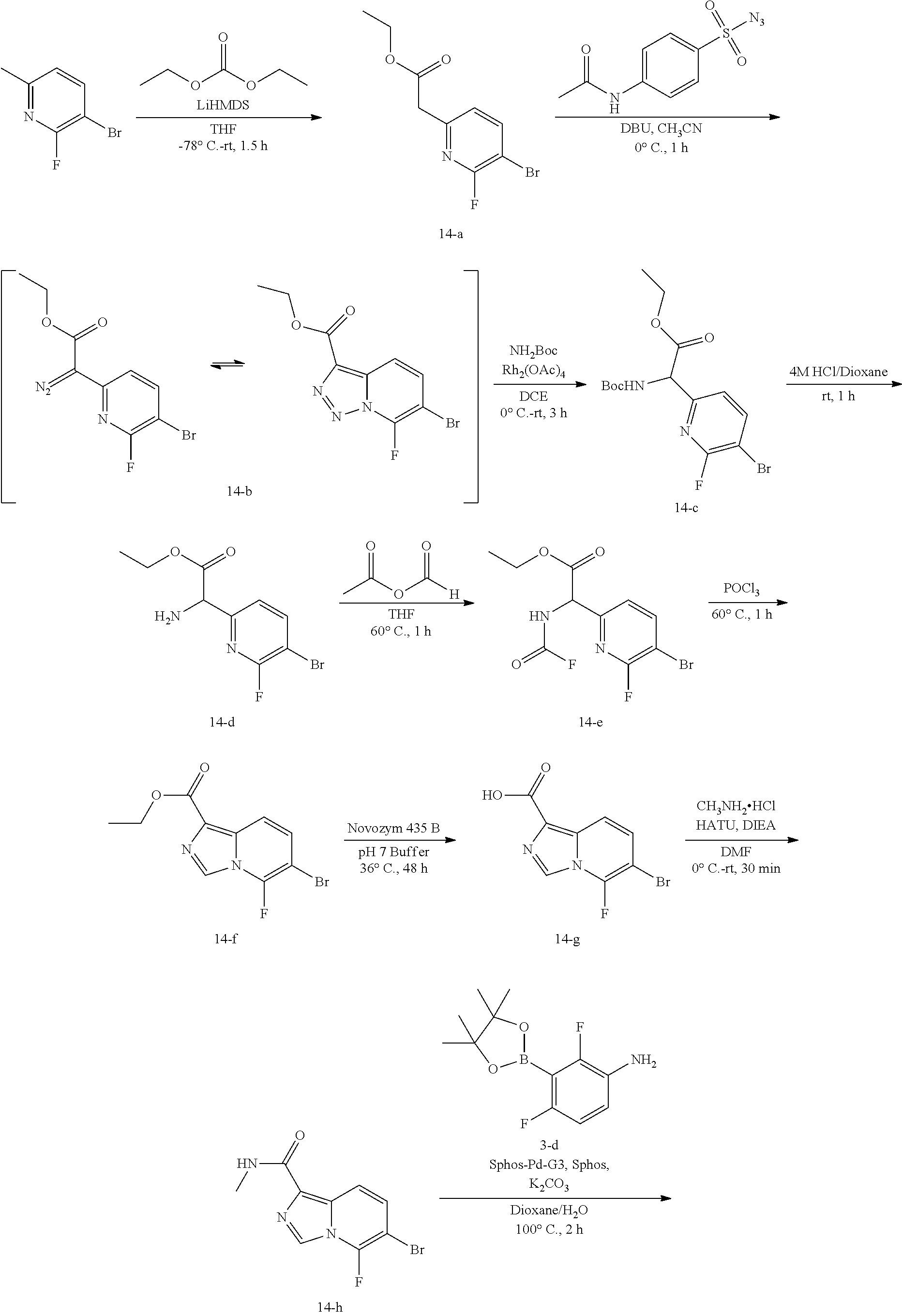

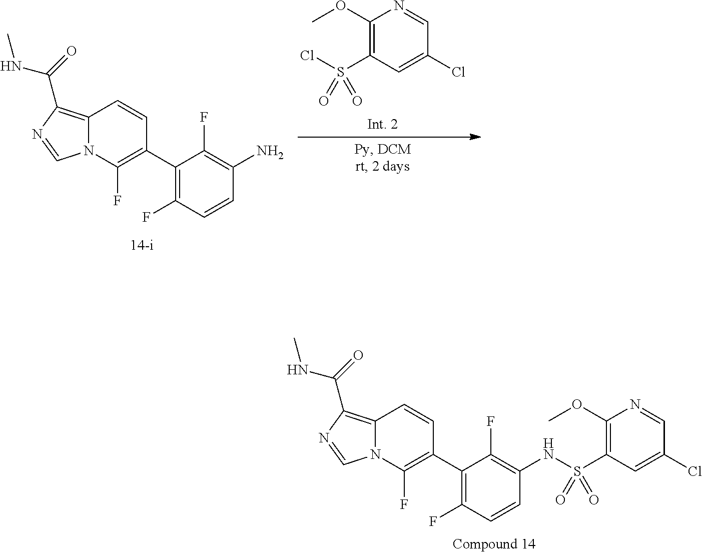 [SMILES: CNC(=O)c1ncn2c(F)c(-c3c(F)ccc(N)c3F)ccc12 ; COc1ncc(Cl)cc1S(=O)(=O)Cl ; CNC(=O)c1ncn2c(F)c(-c3c(F)ccc(NS(=O)(=O)c4cc(Cl)cnc4OC)c3F)ccc12]

### Synthesis of 14-a: ethyl 2-(5-bromo-6-fluoropyridin-2-yl)acetate

To a stirred solution of 3-bromo-2-fluoro-6-methylpyridine (6 g, 31.5 mmol, 1 equiv) in THE (79 mL) was added 1 M LiHMDS solution in THE (63 mL, 63 mmol, 2 equiv) dropwise at −78° C. under N₂ atmosphere. The reaction was stirred at low temperature for 30 min and then diethyl carbonate (5.6 g, 47.4 mmol, 1.5 equiv) was added. The resulting solution was allowed to stir to room temperature for 1 h, then quenched by the addition of H₂O (60 mL) at 0° C. The mixture was extracted with EA (50 mL×3) and the combined organic layers were dried over anhydrous Na₂SO₄. After filtration, the filtrate was concentrated under reduced pressure and the residue purified by silica gel column chromatography, eluting with PE:EA (20:1) to afford ethyl 2-(5-bromo-6-fluoropyridin-2-yl)acetate (6.6 g, 80% yield) as a yellow solid.

LCMS (ES, m/z): [M+H]⁺: 262

### Synthesis of 14-b: ethyl 2-(5-bromo-6-fluoropyridin-2-yl)-2-diazoacetate

To a stirred solution of ethyl 2-(5-bromo-6-fluoropyridin-2-yl)acetate (4.6 g, 17.5 mmol, 1 equiv) in MeCN (60 mL) was added 4-acetamidobenzenesulfonyl azide (4.4 g, 18.4 mmol, 1.05 equiv) and DBU (2.9 g, 19.3 mmol, 1.1 equiv) at 0° C. The reaction was stirred at this temperature for 1 h, then diluted with H₂O (50 mL), and extracted with EA (50 mL×3). The combined organics were dried over anhydrous Na₂SO₄. After filtration, the filtrate was concentrated under reduced pressure, and the residue purified by silica gel column chromatography, eluting with PE:EA (10:1) to afford ethyl 2-(5-bromo-6-fluoropyridin-2-yl)-2-diazoacetate (5 g, 99% yield) as a yellow solid.

LCMS (ES, m/z): [M+H]⁺: 288

### Synthesis of 14-c: ethyl 2-(5-bromo-6-fluoropyridin-2-yl)-2-[(tert-butoxycarbonyl)amino]acetate

To a stirred solution of ethyl 2-(5-bromo-6-fluoropyridin-2-yl)-2-diazoacetate (4.5 g, 15.6 mmol, 1 equiv) in DCE (156 mL) was added tert-butyl carbamate (2.7 g, 23.4 mmol, 1.5 equiv) and Rh₂(OAc)₄ (690 mg, 1.56 mmol, 0.1 equiv) at 0° C. The reaction was stirred at room temperature for 3 h. The resulting solution was concentrated and the residue was purified by silica gel column chromatography, eluting with PE:EA (10:1) to afford ethyl 2-(5-bromo-6-fluoropyridin-2-yl)-2-[(tert-butoxycarbonyl)amino]acetate (5.5 g, 93% yield) as a yellow oil.

LCMS (ES, m/z): [M+H]⁺: 377

### Synthesis of 14-d: ethyl 2-amino-2-(5-bromo-6-fluoropyridin-2-yl)acetate

Ethyl 2-(5-bromo-6-fluoropyridin-2-yl)-2-[(tert-butoxycarbonyl)amino]acetate (7 g) was dissolved in 4 M HCl in 1,4-dioxane (35 mL) and stirred at room temperature for 1 h. The mixture was basified to pH 8 with saturated aqueous NaHCO₃, and extracted with DCM (50 mL×3). The combined organic layers were washed with brine (20 mL) and dried over anhydrous Na₂SO₄. After filtration, the filtrate was concentrated under reduced pressure to give ethyl 2-amino-2-(5-bromo-6-fluoropyridin-2-yl)acetate (5.4 g, crude) as a red oil which was used in the next step without further purification.

LCMS (ES, m/z): [M+H]⁺: 277

### Synthesis of 14-e: ethyl 2-(5-bromo-6-fluoropyridin-2-yl)-2-formamidoacetate

To a stirred solution of ethyl 2-amino-2-(5-bromo-6-fluoropyridin-2-yl)acetate (3 g, 10.8 mmol, 1 equiv) in THE (54 mL) was added acetic formic anhydride (1.4 g, 16.2 mmol, 1.5 equiv). The reaction was stirred at 60° C. for 1 h, then concentrated. The residue was suspended in DCM (50 mL) and washed with H₂O (30 mL×3). The organic layer was dried over anhydrous Na₂SO₄, and concentrated under reduced pressure. The residue was purified by silica gel column chromatography, eluting with PE:EA (3:1) to afford ethyl 2-(5-bromo-6-fluoropyridin-2-yl)-2-formamidoacetate (2 g, 61% yield) as a yellow solid.

LCMS (ES, m/z): [M+H]⁺: 305

### Synthesis of 14-f: ethyl 6-bromo-5-fluoroimidazo[1,5-a]pyridine-1-carboxylate

Ethyl 2-(5-bromo-6-fluoropyridin-2-yl)-2-formamidoacetate (1 g, 3.3 mmol, 1 equiv) was dissolved in POCl₃ (10 mL) in a 40 mL vial and stirred at 60° C. for 1 h. The resulting solution was cooled to room temperature and poured onto ice, then extracted with EA (20 mL×3). The organic layer was dried over anhydrous Na₂SO₄, and concentrated under reduced pressure. The residue was purified by silica gel column chromatography, eluting with PE:EA (5:1) to afford ethyl 6-bromo-5-fluoroimidazo[1,5-a]pyridine-1-carboxylate (0.8 g, 85% yield) as a grey solid.

LCMS (ES, m/z): [M+H]⁺: 287

### Synthesis of 14-g: 6-bromo-5-fluoroimidazo[1,5-a]pyridine-1-carboxylic acid

To a stirred solution of ethyl 6-bromo-5-fluoroimidazo[1,5-a]pyridine-1-carboxylate (200 mg, 0.77 mmol, 1 equiv) in DMSO (2 mL) and pH 7 buffer (4 mL) was added enzyme catalyst Novozym 435 B (100 mg). The reaction was stirred at 36° C. for 48 h, then filtered. The filtrate was concentrated and the residue purified by Flash-Prep-HPLC with the following conditions: Column, WelFlash™ C18-I, Spherical C18 20-40 m, 120 g; mobile phase, 40-80% MeCN over 20 min/0.1% aqueous formic acid; Detector, 220 nm; to give 6-bromo-5-fluoroimidazo[1,5-a]pyridine-1-carboxylic acid (86 mg, 48% yield) as a grey solid.

LCMS (ES, m/z): [M+H]⁺: 259.

### Synthesis of 14-h: 6-bromo-5-fluoro-N-methylimidazo[1,5-a]pyridine-1-carboxamide

To a stirred solution of 6-bromo-5-fluoroimidazo[1,5-a]pyridine-1-carboxylic acid (70 mg, 0.27 mmol, 1 equiv) in DMF (2 mL) was added HATU (123 mg, 0.32 mmol, 1.2 equiv) and DIEA (105 mg, 0.81 mmol, 3 equiv). The reaction was stirred at room temperature for 30 min and then cooled to 0° C. in an ice bath. Methylamine hydrochloride (18 mg, 0.27 mmol, 1 equiv) was added and the resulting solution was stirred at rt for another 30 min. The resulting mixture was diluted with H₂O (3 mL), and the aqueous layer extracted with EA (5 mL×3). The combined organics were concentrated and purified by silica gel column chromatography, eluting with PE:EA (1:2) to afford 6-bromo-5-fluoro-N-methylimidazo[1,5-a]pyridine-1-carboxamide (70 mg, 95% yield) as a white solid.

LCMS (ES, m/z): [M+H]⁺: 272.

### Synthesis of 14-i: 6-(3-amino-2,6-difluorophenyl)-5-fluoro-N-methylimidazo[1,5-a]pyridine-1-carboxamide

To a solution of 6-bromo-5-fluoro-N-methylimidazo[1,5-a]pyridine-1-carboxamide (100 mg, 0.37 mmol, 1 equiv) and 2,4-difluoro-3-(4,4,5,5-tetramethyl-1,3,2-dioxaborolan-2-yl)aniline (188 mg, 0.74 mmol, 2 equiv) in dioxane (2 mL) and H₂O (0.2 mL) were added K₂CO₃ (127 mg, 0.92 mmol, 2.5 equiv), S-Phos (30 mg, 0.074 mmol, 0.2 equiv) and SPhos Pd Gen.3 (29 mg, 0.037 mmol, 0.1 equiv). The reaction was stirred at 100° C. for 2 h under nitrogen atmosphere. The resulting mixture was filtered and the filter cake was washed with dioxane (5 mL). The filtrate was concentrated under reduced pressure and the residue purified by Flash-Prep-HPLC with the following conditions: Column, WelFlash™ C18-I, Spherical C18 20-40 m, 120 g; mobile phase, 40-80% MeCN over 20 min/0.1% aqueous formic acid; Detector, 220 nm; to give 6-(3-amino-2,6-difluorophenyl)-5-fluoro-N-methylimidazo[1,5-a]pyridine-1-carboxamide (90 mg, 76% yield) as a white solid.

LCMS (ES, m/z): [M+H]⁺: 321.

### Synthesis of Compound 14: 6-[3-(5-chloro-2-methoxypyridine-3-sulfonamido)-2,6-difluorophenyl]-5-fluoro-N-methylimidazo[1,5-a]pyridine-1-carboxamide

To a stirred solution of 6-(3-amino-2,6-difluorophenyl)-5-fluoro-N-methylimidazo[1,5-a]pyridine-1-carboxamide (60 mg, 0.19 mmol, 1 equiv) in DCM (3 mL) was added 5-chloro-2-methoxypyridine-3-sulfonyl chloride (54 mg, 0.23 mmol, 1.2 equiv) and pyridine (29 mg, 0.38 mmol, 2 equiv). The mixture was stirred at room temperature for 2 days. The solution was concentrated directly and the residue was purified by prep-HPLC with the following conditions: Column, welch Vltimate XB-C18, 50×250 mm, 10 μm mobile phase; Mobile Phase: 30-70% MeCN over 15 min/0.1% aqueous formic acid; Detector, 220 nm; to give 6-[3-(5-chloro-2-methoxypyridine-3-sulfonamido)-2,6-difluorophenyl]-5-fluoro-N-methylimidazo[1,5-a]pyridine-1-carboxamide (40 mg, 41% yield) as a white solid.

LCMS (ES, m/z): [M+H]⁺: 526.

¹H NMR (300 MHz, DMSO-d₆) δ 10.51 (s, 1H), 8.67 (d, J=0.7 Hz, 1H), 8.52 (d, J=2.6 Hz, 1H), 8.24 (d, J=4.8 Hz, 1H), 8.08 (d, J=2.6 Hz, 1H), 8.01 (d, 1H), 7.50-7.44 (m, 1H), 7.36-7.26 (m, 1H), 7.18-7.07 (m, 1H), 3.91 (s, 3H), 2.82 (d, J=4.7 Hz, 3H).

### Example 30: Synthesis of 6-[3-(5-chloro-2-methoxypyridine-3-sulfonamido)-2,6-difluorophenyl]-5-methoxy-N-methylimidazo[1,5-a]pyridine-1-carboxamide (Compound 15)

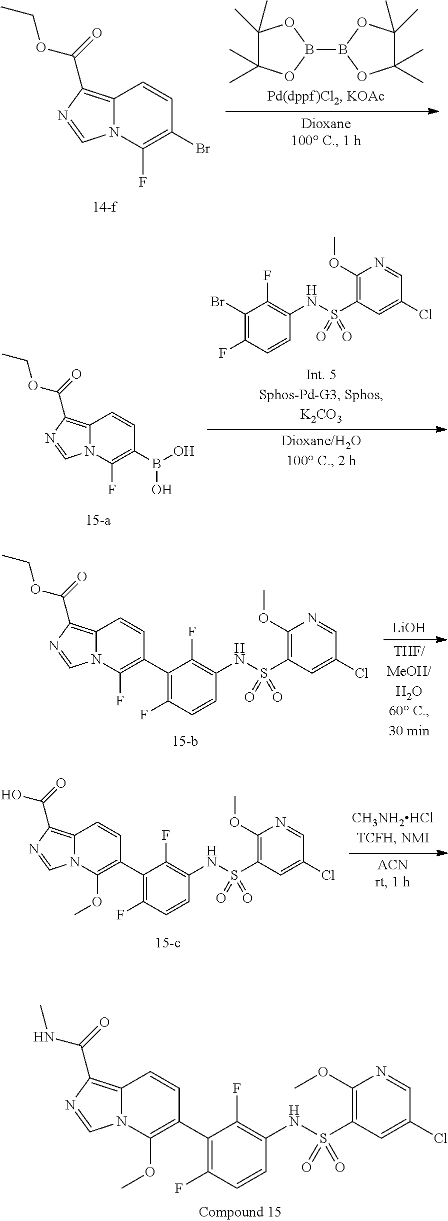

### Synthesis of 15-a: 1-(ethoxycarbonyl)-5-fluoroimidazo[1,5-a]pyridin-6-ylboronic acid

To a stirred solution of 6-bromo-5-fluoroimidazo[1,5-a]pyridine-1-carboxylate (1 g, 3.5 mmol, 1 equiv) and bis(pinacolato)diboron (1.8 g, 7 mmol, 2 equiv) in dioxane (18 mL) was added KOAc (684 mg, 7 mmol, 2 equiv) an d Pd(dppf)Cl₂ (255 mg, 0.35 mmol, 0.1 equiv). After stirring for 1 h at 100° C. under nitrogen atmosphere, the mixture was concentrated, and the residue was purified by Flash-Prep-HPLC with the following conditions: Column, WelFlash™ C18-I, Spherical C18 20-40 m, 120 g; mobile phase, 5-45% MeCN/0.1% aqueous formic acid, over 25 min; Detector, 220 nm; to give 1-(ethoxycarbonyl)-5-fluoroimidazo[1,5-a]pyridin-6-ylboronic acid (400 mg, 45% yield) as a white solid.

LCMS (ES, m/z): [M+H]⁺: 253

### Synthesis of 15-b: ethyl 6-[3-(5-chloro-2-methoxypyridine-3-sulfonamido)-2,6-difluorophenyl]-5-fluoroimidazo[1,5-a]pyridine-1-carboxylate

To a stirred solution of 1-(ethoxycarbonyl)-5-fluoroimidazo[1,5-a]pyridin-6-ylboronic acid (400 mg, 1.6 mmol, 1 equiv) and N-(3-bromo-2,4-difluorophenyl)-5-chloro-2-methoxypyridine-3-sulfonamide (788 mg, 1.9 mmol, 1.2 equiv) in dioxane (7 mL) and H₂O (1.4 mL) were added K₂CO₃ (548 mg, 4 mmol, 2.5 equiv), SPhos Pd Gen.3 (124 mg, 0.16 mmol, 0.1 equiv) and S-Phos (130 mg, 0.32 mmol, 0.2 equiv).

After stirring for 2 h at 100° C. under a nitrogen atmosphere, the resulting mixture was concentrated under reduced pressure. The residue was purified by silica gel column chromatography, eluting with PE:EA (1:1) to afford ethyl 6-[3-(5-chloro-2-methoxypyridine-3-sulfonamido)-2,6-difluorophenyl]-5-fluoroimidazo[1,5-a]pyridine-1-carboxylate (80 mg, 9% yield) as a red solid.

LCMS (ES, m/z): [M+H]⁺: 541

### Synthesis of 15-c: 6-[3-(5-chloro-2-methoxypyridine-3-sulfonamido)-2,6-difluorophenyl]-5-methoxyimidazo[1,5-a]pyridine-1-carboxylic acid

To a stirred solution of ethyl 6-[3-(5-chloro-2-methoxypyridine-3-sulfonamido)-2,6-difluorophenyl]-5-fluoroimidazo[1,5-a]pyridine-1-carboxylate (60 mg, 0.11 mmol, 1 equiv) in a mixture of THE (2 mL), MeOH (2 mL) and H₂O (1 mL) was added LiOH (8 mg, 0.33 mmol, 3 equiv). The reaction was then stirred at 60° C. for 30 min. The mixture was concentrated under vacuum, and the residue was purified by Flash-Prep-HPLC with the following conditions: Column, WelFlash™ C18-I, Spherical C18 20-40 m, 120 g; mobile phase, 40-70% MeCN/0.1% aqueous formic acid over 15 min; Detector, 220 nm; to give 6-[3-(5-chloro-2-methoxypyridine-3-sulfonamido)-2,6-difluorophenyl]-5-methoxyimidazo[1,5-a]pyridine-1-carboxylic acid (40 mg, 69% yield) as a white solid.

LCMS (ES, m/z): [M+H]⁺: 525

### Synthesis of Compound 15: 6-[3-(5-chloro-2-methoxypyridine-3-sulfonamido)-2,6-difluorophenyl]-5-methoxy-N-methylimidazo[1,5-a]pyridine-1-carboxamide

To a stirred solution of 6-[3-(5-chloro-2-methoxypyridine-3-sulfonamido)-2,6-difluorophenyl]-5-methoxyimidazo[1,5-a]pyridine-1-carboxylic acid (70 mg, 0.13 mmol, 1 equiv) in MeCN (2 mL) was added TCFH (41 mg, 0.15 mmol, 1.1 equiv), NMI (33 mg, 0.4 mmol, 3 equiv) and CH₃NH₂·HCl (9 mg, 0.13 mmol, 1 equiv). The reaction was stirred at room temperature for 1 h.

The resulting mixture was diluted with H₂O (3 mL) and extracted with EA (5 mL×3). The combined organic layers were dried over anhydrous sodium sulphate and concentrated under reduced pressure. The residue was purified by prep-HPLC with the following conditions: Column, welch Vltimate XB-C18, 50×250 mm, 10 μm mobile phase, Mobile Phase A: 0.1% aqueous formic acid, Mobile Phase B: MeCN (30-70% over 15 min) Detector, 220 nm; to give 6-[3-(5-chloro-2-methoxypyridine-3-sulfonamido)-2,6-difluorophenyl]-5-methoxy-N-methylimidazo[1,5-a]pyridine-1-carboxamide (10 mg, 14% yield) as a grey solid.

LCMS (ES, m/z): [M+H]⁺: 538.

¹H NMR (300 MHz, Methanol-d₄) δ 8.39 (s, 1H), 8.37 (d, J=2.6 Hz, 1H), 8.06 (d, J=2.6 Hz, 1H), 7.96 (d, J=9.3 Hz, 1H), 7.66-7.58 (m, 1H), 7.15 (t, J=8.6 Hz, 1H), 6.94 (d, J=9.3 Hz, 1H), 4.06 (s, 3H), 3.65 (s, 3H), 2.99 (s, 3H).

### Example 31: Synthesis of 3-[3-(5-Chloro-2-methoxypyridine-3-sulfonamido)-2,6-difluorophenyl]-N-methylimidazo[1,5-a]pyrimidine-8-carboxamide (Compound 16)

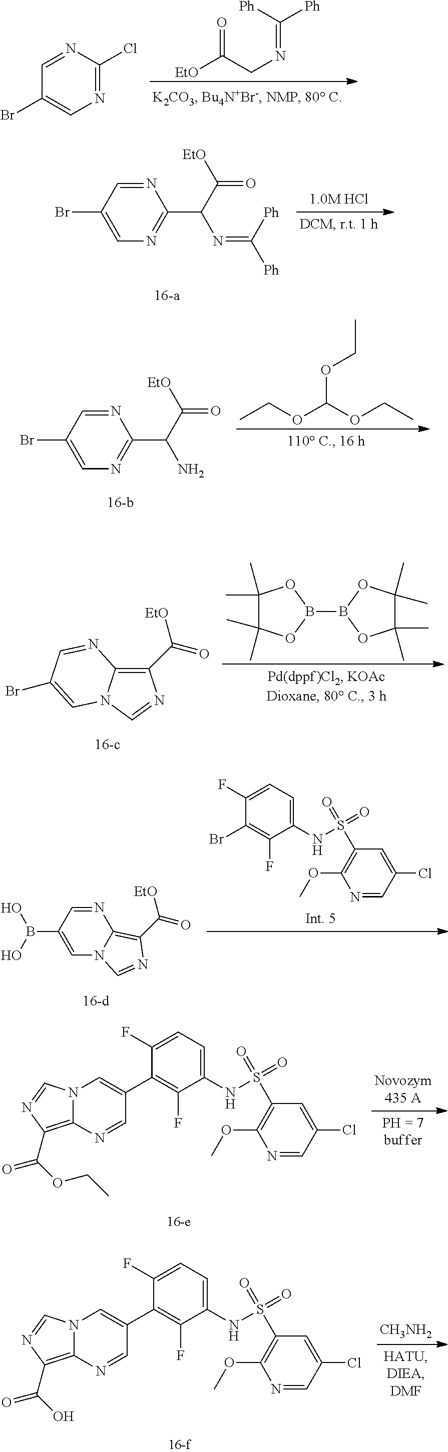

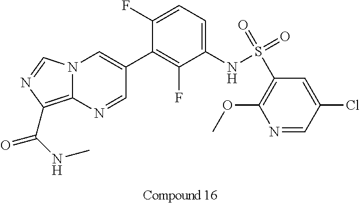 [SMILES: CNC(=O)c1ncn2cc(-c3c(F)ccc(NS(=O)(=O)c4cc(Cl)cnc4OC)c3F)cnc12]

### Synthesis of 16-a: 2-(5-bromopyrimidin-2-yl)-2-|(diphenylmethylidene)amino]acetate

Into a 250 mL 3-necked round-bottom flask, was placed 5-bromo-2-chloropyrimidine (10 g, 52 mmol, 1 equiv), ethyl 2-[(diphenylmethylidene)amino]acetate (13.8 g, 52 mmol, 1 equiv), K₂CO₃ (21.4 g, 155 mmol, 3 equiv), Bu₄NBr (16.6 g, 52 mmol, 1 equiv) and NMP (100 mL). The resulting solution was stirred for 16 h at 80° C. in an oil bath. The reaction was then quenched by the addition of 100 mL of water/ice. The resulting solution was extracted with 3×200 mL of dichloromethane and the organics were washed with 2×100 ml of brine. The mixture was dried over anhydrous sodium sulfate and concentrated under vacuum. The residue was applied onto a silica gel column, eluting with ethyl acetate/petroleum ether (1:5) to give ethyl 2-(5-bromopyrimidin-2-yl)-2-[(diphenylmethylidene)amino]acetate (5.5 g, 25% yield) as a white solid.

LCMS (ES, m/z): [M+H]⁺: 424

### Synthesis of 16-b: ethyl 2-amino-2-(5-bromopyrimidin-2-yl)acetate

Into a 250 mL 3-necked round-bottom flask, was placed ethyl 2-(5-bromopyrimidin-2-yl)-2-[(diphenylmethylidene)amino]acetate (5.5 g, 13 mmol, 1 equiv) in DCM (55 mL).

This was followed by the addition of HCl (1 M) (10 mL) dropwise with stirring at room temperature. The resulting solution was stirred for 1 hr, then diluted with 100 mL of water. The pH of the solution was adjusted to 8 with NaHCO₃ (1M), then was extracted with 3×50 mL of dichloromethane. The combined organic layers were dried over anhydrous sodium sulfate and concentrated under vacuum. The residue was applied onto a silica gel column, eluting with ethyl acetate/petroleum ether (1:5) to give ethyl 2-amino-2-(5-bromopyrimidin-2-yl)acetate (2.2 g, 65% yield) as a yellow solid.

LCMS (ES, m/z): [M+H]+: 260

### Synthesis of 16-c: ethyl 3-bromoimidazo[1,5-a]pyrimidine-8-carboxylate

Into a 100 mL 3-necked round-bottom flask, was placed ethyl 2-amino-2-(5-bromopyrimidin-2-yl)acetate (2 g, 7.6 mmol, 1 equiv) and triethyl orthoformate (20 mL). The resulting solution was stirred for 16 h at 110° C. in an oil bath. The mixture was cooled to room temperature and diluted with 10 mL of PE. The solids were collected by filtration and dried to give ethyl 3-bromoimidazo[1,5-a]pyrimidine-8-carboxylate (2.0 g, 96% yield) as a white solid.

LCMS (ES, m/z): [M+H]⁺: 270

### Synthesis of 16-d: 8-(ethoxycarbonyl)imidazo[1,5-a]pyrimidin-3-ylboronic acid

Into a 100 mL 3-necked round-bottom flask, purged and maintained with an inert atmosphere of nitrogen, was placed ethyl 3-bromoimidazo[1,5-a]pyrimidine-8-carboxylate (2 g, 7.4 mmol, 1 equiv), bis(pinacolato)diboron (2.8 g, 11 mmol, 1.5 equiv), dioxane (20 mL), KOAc (1.45 g, 15 mmol, 2 equiv) and Pd(dppf)Cl₂ (1.08 g, 1.5 mmol, 0.2 equiv). The resulting solution was stirred for 3 hr at 80° C. in an oil bath. The solids were removed by filtration and the filtrate concentrated under vacuum. The crude product was purified by prep-HPLC with the following conditions: Column, WelFlash™ C18-I, Spherical C18 20-40 m, 330 g; Mobile Phase: 5% MeCN/0.1% aqueous formic acid; Detector, 220 nm; to give 8-(ethoxycarbonyl)imidazo[1,5-a]pyrimidin-3-ylboronic acid (1.6 g, 92% yield) as a yellow solid.

LCMS (ES, m/z): [M+H]⁺: 236

### Synthesis of 16-e: ethyl 3-[3-(5-chloro-2-methoxypyridine-3-sulfonamido)-2,6-difluorophenyl]imidazo[1,5-a]pyrimidine-8-carboxylate

Into a 25 mL 3-necked round-bottom flask purged and maintained with an inert atmosphere of nitrogen, was placed 8-(ethoxycarbonyl)imidazo[1,5-a]pyrimidin-3-ylboronic acid (255 mg, 1.9 mmol, 1.5 equiv), N-(3-bromo-2,4-difluorophenyl)-5-chloro-2-methoxypyridine-3-sulfonamide (300 mg, 1.3 mmol, 1 equiv), dioxane (10 mL), H₂O (1 mL), K₂CO₃ (200 mg, 2.6 mmol, 2 equiv) and Pd(dppf)Cl₂ (53 mg, 0.07 mol, 0.1 equiv). The resulting solution was stirred for 3 hr at 80° C. in an oil bath. The reaction mixture was cooled to room temperature and filtered. The filtrate was concentrated under vacuum and the residue was purified by prep-HPLC with the following conditions: Column, WelFlash™ C18-I, Spherical C18 20-40 m, 120 g; Mobile Phase: 70-95% MeCN over 12 mins/0.1% aqueous formic acid; Detector, 220 nm. Ethyl 3-[3-(5-chloro-2-methoxypyridine-3-sulfonamido)-2,6-difluorophenyl]imidazo[1,5-a]pyrimidine-8-carboxylate (150 mg, 39% yield) was isolated as a yellow solid.

LCMS (ES, m/z): [M+H]⁺: 524

### Synthesis of 16-f: 3-[3-(5-chloro-2-methoxypyridine-3-sulfonamido)-2,6-difluorophenyl]imidazo[1,5-a]pyrimidine-8-carboxylic acid

Into an 8 mL round-bottom flask, was placed ethyl 3-[3-(5-chloro-2-methoxypyridine-3-sulfonamido)-2,6-difluorophenyl]imidazo[1,5-a]pyrimidine-8-carboxylate (600 mg, 1.1 mmol, 1 equiv), Novozym 435 A (200% SM) and pH 7 buffer (10 mL). The resulting solution was stirred for 24 h at 36° C. The mixture was filtered and the filtrate concentrated. The residue was purified by Prep-HPLC with the following conditions: Column, welch Vltimate XB-C18, 50×250 mm, 10 μm mobile phase: Mobile Phase 10-65% MeCN over 12 mins/0.1% aqueous formic acid; to yield 3-[3-(5-chloro-2-methoxypyridine-3-sulfonamido)-2,6-difluorophenyl]imidazo[1,5-a]pyrimidine-8-carboxylic acid (200 mg, 35% yield) as a white solid.

LCMS (ES, m/z): [M+H]⁺: 496

### Synthesis of Compound 16: 3-[3-(5-Chloro-2-methoxypyridine-3-sulfonamido)-2,6-difluorophenyl]-N-methylimidazo[1,5-a]pyrimidine-8-carboxamide

Into a 25 mL round-bottom flask, was placed 3-[3-(5-chloro-2-methoxypyridine-3-sulfonamido)-2,6-difluorophenyl]imidazo[1,5-a]pyrimidine-8-carboxylic acid (200 mg, 0.4 mmol, 1 equiv), DMF (5 mL), HATU (199 mg, 0.5 mmol, 1.3 equiv), DIEA (104 mg, 0.8 mmol, 2 equiv) and methylamine (25.05 mg, 0.8 mmol, 2 equiv). The resulting solution was stirred for 3 hr at room temperature. The reaction was quenched by the addition of 10 mL of water and extracted with 3×10 mL of dichloromethane. The combined organics were washed with 1×10 ml of brine, dried over anhydrous sodium sulfate and concentrated under vacuum. The residue was purified by Prep-HPLC with the following conditions: Column, welch Vltimate XB-C18, 50×250 mm, 10 μm; mobile phase 30-60% MeCN over 15 mins/0.1% aqueous formic acid; Detector, UV. 3-[3-(5-Chloro-2-methoxypyridine-3-sulfonamido)-2,6-difluorophenyl]-N-methylimidazo[1,5-a]pyrimidine-8-carboxamide (105 mg, 51% yield) was obtained as a white solid.

LCMS (ES, m/z): [M+H]⁺: 509

¹H NMR (300 MHz, DMSO-d₆) δ 10.52 (s, 1H), 9.05 (d, J=2.2 Hz, 1H), 8.52 (d, J=2.6 Hz, 1H), 8.49-8.39 (m, 2H), 8.10 (d, J=2.6 Hz, 1H), 8.03 (d, J=4.9 Hz, 1H), 7.44-7.39 (m, 1H), 7.36-7.27 (m, 1H), 3.94 (s, 3H), 2.85 (d, J=4.7 Hz, 3H).

### Example 32: Synthesis of 3-[3-(5-chloro-2-methoxypyridine-3-sulfonamido)-2,6-difluorophenyl]-2-methoxy-N-methylimidazo[1,5-a]pyrimidine-8-carboxamide (Compound 17)

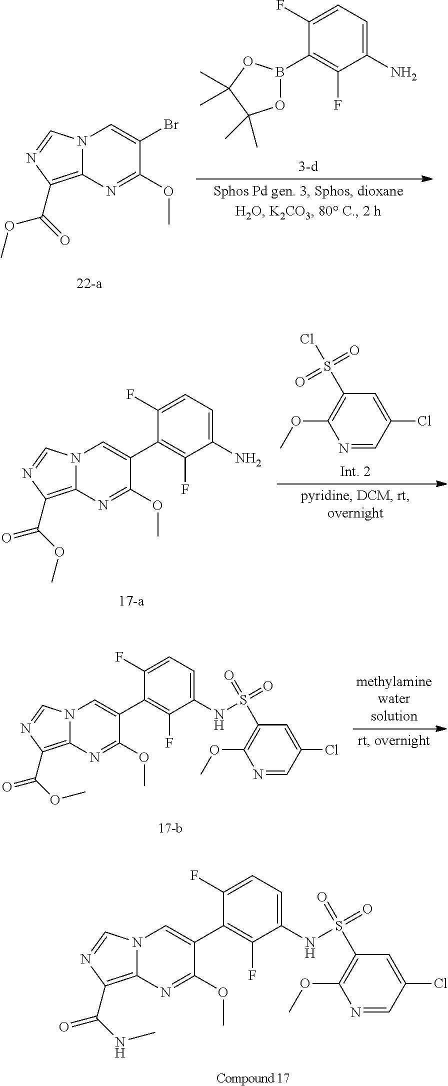

### Synthesis of 17-a: methyl 3-(3-amino-2,6-difluorophenyl)-2-methoxyimidazo[1,5-a]pyrimidine-8-carboxylate

Into a 40 mL vial, purged and maintained with an inert atmosphere of nitrogen, was placed methyl 3-bromo-2-methoxyimidazo[1,5-a]pyrimidine-8-carboxylate (500 mg, 1.7 mmol, 1 equiv), 2,4-difluoro-3-(4,4,5,5-tetramethyl-1,3,2-dioxaborolan-2-yl)aniline (891 mg, 3.5 mmol, 2 equiv), SphosPdG3 (136 mg, 0.2 mmol, 0.1 equiv), Sphos (143 mg, 0.3 mmol, 0.2 equiv), dioxane (8 mL), H₂O (1.6 mL) and K₂CO₃ (724 mg, 5.2 mmol, 3 equiv). The resulting solution was stirred for 2 h at 80° C. in an oil bath. The reaction mixture was cooled and concentrated. The residue was dissolved in 50 mL of DCM and the mixture filtered. The filtrate was dried over anhydrous sodium sulfate and concentrated. The residue was applied onto a silica gel column, eluting with ethyl acetate/petroleum ether (1:1) to give methyl 3-(3-amino-2,6-difluorophenyl)-2-methoxyimidazo[1,5-a]pyrimidine-8-carboxylate (100 mg, 17% yield) as a yellow solid.

LCMS (ES, m/z): [M+H]⁺: 335

### Synthesis of 17-b: methyl 3-[3-(5-chloro-2-methoxypyridine-3-sulfonamido)-2,6-difluorophenyl]-2-methoxyimidazo[1,5-a]pyrimidine-8-carboxylate

Into an 8 mL vial, was placed methyl 3-(3-amino-2,6-difluorophenyl)-2-methoxyimidazo[1,5-a]pyrimidine-8-carboxylate (70 mg, 0.2 mmol, 1 equiv), DCM (2 mL), pyridine (83 mg, 1 mmol, 5 equiv) and 5-chloro-2-methoxypyridine-3-sulfonyl chloride (61 mg, 0.3 mmol, 1.2 equiv). The resulting solution was stirred for overnight at 25° C. The resulting mixture was concentrated and the crude product was purified by Flash-Prep-HPLC with the following conditions: Column, WelFlash™ C18-I, Spherical C18 20-40 m, 120 g; mobile phase 5-60% MeCN over 20 min/0.1% aqueous formic acid; Detector, 220 nm. Methyl 3-[3-(5-chloro-2-methoxypyridine-3-sulfonamido)-2,6-difluorophenyl]-2-methoxyimidazo[1,5-a]pyrimidine-8-carboxylate (95 mg, 84% yield) was isolated as a white solid.

LCMS (ES, m/z): [M+H]⁺: 540

### Synthesis of Compound 17: 3-[3-(5-chloro-2-methoxypyridine-3-sulfonamido)-2,6-difluorophenyl]-2-methoxy-N-methylimidazo[1,5-a]pyrimidine-8-carboxamide

Into a 50 mL round-bottom flask, was placed methyl 3-[3-(5-chloro-2-methoxypyridine-3-sulfonamido)-2,6-difluorophenyl]-2-methoxyimidazo[1,5-a]pyrimidine-8-carboxylate (80 mg, 0.15 mmol, 1 equiv) and 40% aqueous methylamine solution (5 mL). The resulting solution was stirred for overnight at 25° C. and then concentrated. The crude product was purified by Prep-HPLC with the following conditions: Column, welch Vltimate XB-C18, 50×250 mm, 10 μm mobile phase, Mobile Phase 33-53% MeCN over 20 min/0.1% aqueous formic acid; Detector, 220 nm; to give 3-[3-(5-chloro-2-methoxypyridine-3-sulfonamido)-2,6-difluorophenyl]-2-methoxy-N-methylimidazo[1,5-a]pyrimidine-8-carboxamide (38 mg, 48% yield) as a white solid.

LCMS (ES, m/z): [M+H]⁺: 539

¹H NMR (300 MHz, DMSO-d₆) δ 10.45 (s, 1H), 8.90 (s, 1H), 8.52 (d, J=2.6 Hz, 1H), 8.18 (s, 1H), 8.08 (d, J=2.6 Hz, 1H), 7.77 (d, J=4.9 Hz, 1H), 7.45 (td, J=8.9, 5.9 Hz, 1H), 7.24 (t, J=8.9 Hz, 1H), 3.92 (d, J=2.1 Hz, 6H), 2.83 (d, J=4.7 Hz, 3H).

### Example 33: Synthesis of N-[2,4-difluoro-3-[1-(1H-imidazol-2-yl)imidazo[1,5-a]pyridin-6-yl]phenyl]-5-fluoro-2-methoxypyridine-3-sulfonamide (Compound 18)

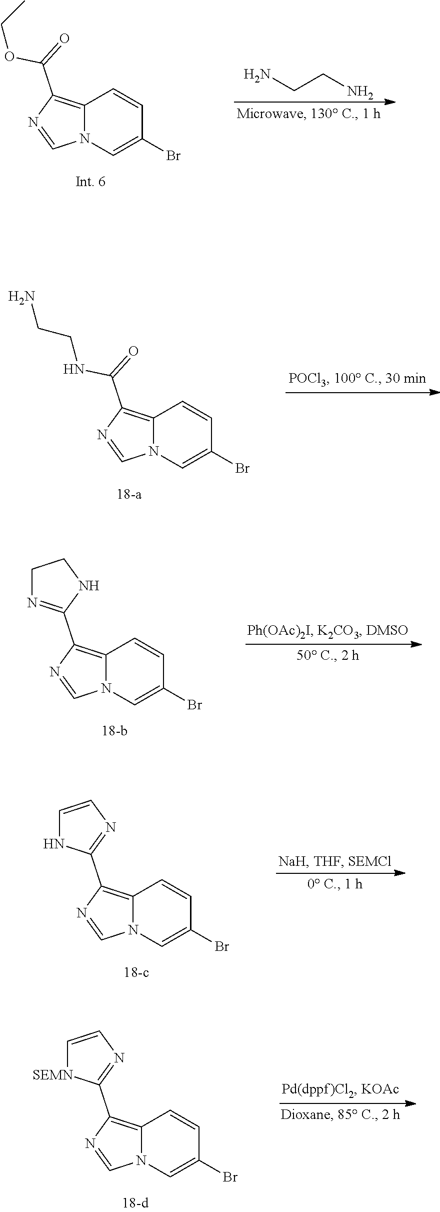

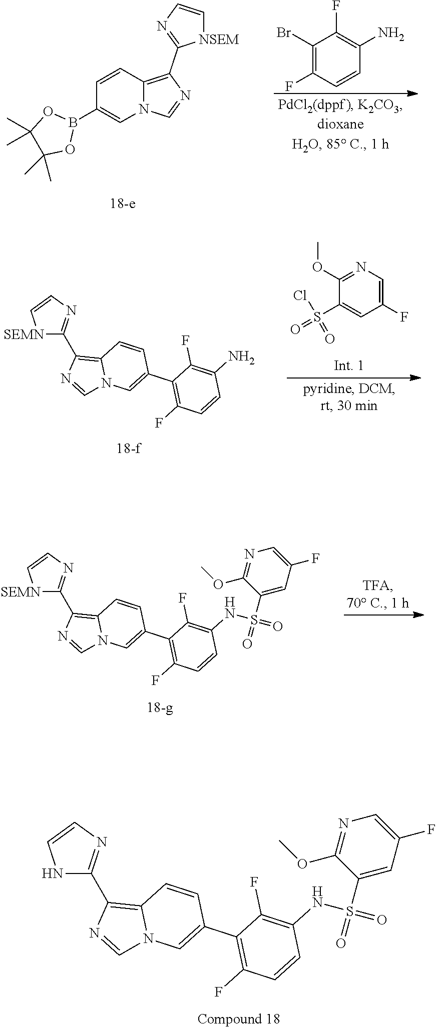 [SMILES: CC1(C)OB(c2ccc3c(-c4nccn4COCC[Si](C)(C)C)ncn3c2)OC1(C)C ; Nc1ccc(F)c(Br)c1F ; C[Si](C)(C)CCOCn1ccnc1-c1ncn2cc(-c3c(F)ccc(N)c3F)ccc12 ; COc1ncc(F)cc1S(=O)(=O)Cl ; COc1ncc(F)cc1S(=O)(=O)Nc1ccc(F)c(-c2ccc3c(-c4nccn4COCC[Si](C)(C)C)ncn3c2)c1F ; COc1ncc(F)cc1S(=O)(=O)Nc1ccc(F)c(-c2ccc3c(-c4ncc[nH]4)ncn3c2)c1F]

### Synthesis of 18-a: N-(2-aminoethyl)-6-bromoimidazo[1,5-a]pyridine-1-carboxamide

Into a 30 mL microwave vial was placed ethyl 6-bromoimidazo[1,5-a]pyridine-1-carboxylate (5 g, 19 mmol, 1 equiv) and ethylenediamine (10 mL). The resulting solution was stirred for 1 h at 130° C. The resulting mixture was concentrated under vacuum to give N-(2-aminoethyl)-6-bromoimidazo[1,5-a]pyridine-1-carboxamide (2.07 g, crude) as a brown solid, which was used in next step without any purification.

LCMS (ES, m/z): [M+H]⁺: 283

### Synthesis of 18-b: 2-[6-bromoimidazo[1,5-a]pyridin-1-yl]-4,5-dihydro-1H-imidazole

Into a 25 mL 3-necked round-bottom flask, was placed N-(2-aminoethyl)-6-bromoimidazo[1,5-a]pyridine-1-carboxamide (2.07 g, 7.3 mmol, 1 equiv) and POCl₃ (5 mL). The resulting solution was stirred for 30 min at 100° C., then cooled and concentrated under vacuum. The residue was carefully diluted with 50 mL of H₂O and the pH adjusted 8 with saturated aqueous NaHCO₃ solution. This was extracted with 5×100 mL of dichloromethane. The combined organics were dried over anhydrous sodium sulfate and concentrated to give 2-[6-bromoimidazo[1,5-a]pyridin-1-yl]-4,5-dihydro-1H-imidazole (1.0 g, crude) as a brown solid, which was used in next step without any purification.

LCMS (ES, m/z): [M+H]⁺: 265

### Synthesis of 18-c: 2-[6-bromoimidazo[1,5-a]pyridin-1-yl]-1H-imidazole

Into a 25 mL 3-necked round-bottom flask, was placed 2-[6-bromoimidazo[1,5-a]pyridin-1-yl]-4,5-dihydro-1H-imidazole (1 g, 3.8 mmol, 1 equiv), K₂CO₃ (784 mg, 5.7 mmol, 1.5 equiv), PhI(OAc)₂ (1.83 g, 5.7 mmol, 1.5 equiv) and DMSO (10 mL). The resulting solution was stirred for 3 h at 50° C. in an oil bath, then cooled and diluted with 50 mL of H₂O. The resulting solution was extracted with 5×50 mL of dichloromethane. The combined organics were dried over anhydrous sodium sulfate and concentrated under vacuum to give 2-[6-bromoimidazo[1,5-a]pyridin-1-yl]-1H-imidazole (400 mg, crude) as a brown solid, which was used in next step without any purification.

LCMS (ES, m/z): [M+H]⁺: 263

### Synthesis of 18-d: 2-[6-bromoimidazo[1,5-a]pyridin-1-yl]-1-[[2-(trimethylsilyl)ethoxy]methyl]imidazole

Into a 25 mL 3-necked round-bottom flask, purged and maintained with an inert atmosphere of nitrogen, was placed 2-[6-bromoimidazo[1,5-a]pyridin-1-yl]-1H-imidazole (400 mg, 1.52 mmol, 1 equiv) in THE (5 mL). This was followed by the addition of NaH (73 mg, 3 mmol, 2 equiv, 60% in oil) at 0° C. The suspension was stirred for 15 mins, then SEMCI (380 mg, 2.3 mmol, 1.5 equiv) was added at 0° C. The resulting solution was stirred for 1 h in an ice/salt bath. The reaction was quenched with 5 mL of H₂O and concentrated under vacuum. The residue was purified by silica gel column chromatography, eluting with PE:EA (2:1). Concentration of the relevant fractions afforded 2-[6-bromoimidazo[1,5-a]pyridin-1-yl]-1-[[2-(trimethylsilyl)ethoxy]methyl]imidazole (403 mg, 67% yield) as a yellow solid.

LCMS (ES, m/z): [M+H]⁺: 393

### Synthesis of 18-e: 2-[6-(4,4,5,5-tetramethyl-1,3,2-dioxaborolan-2-yl)imidazo[1,5-a]pyridin-1-yl]-1-[[2-(trimethylsilyl)ethoxy]methyl]imidazole

Into a 25 mL 3-necked round-bottom flask, purged and maintained with an inert atmosphere of nitrogen, was placed 2-[6-bromoimidazo[1,5-a]pyridin-1-yl]-1-[[2-(trimethylsilyl)ethoxy]methyl]imidazole (403 mg, 1 mmol, 1 equiv), bis(pinacolato)diboron (390 mg, 1.5 mmol, 1.5 equiv), Pd(dppf)Cl₂ (75 mg, 0.102 mmol, 0.1 equiv), KOAc (302 mg, 3.1 mmol, 3 equiv) and dioxane (5 mL). The resulting solution was stirred for 2 h at 85° C. in an oil bath. The reaction was quenched by the addition of 50 mL of water, and extracted with 2×50 mL of dichloromethane. The combined organics were dried over anhydrous sodium sulfate and concentrated under vacuum to give 2-[6-(4,4,5,5-tetramethyl-1,3,2-dioxaborolan-2-yl)imidazo[1,5-a]pyridin-1-yl]-1-[[2-(trimethylsilyl)ethoxy]methyl]imidazole (532 mg, crude) as brown oil.

LCMS (ES, m/z): [M+H]⁺: 441

### Synthesis of 18-f: 2,4-difluoro-3-[1-(1-[[2-(trimethylsilyl)ethoxy]methyl]imidazol-2-yl)imidazo[1,5-a]pyridin-6-yl]aniline

Into a 25 mL 3-necked round-bottom flask, purged and maintained with an inert atmosphere of nitrogen, was placed 2-[6-(4,4,5,5-tetramethyl-1,3,2-dioxaborolan-2-yl)imidazo[1,5-a]pyridin-1-yl]-1-[[2-(trimethylsilyl)ethoxy]methyl]imidazole (380 mg, 0.9 mmol, 1 equiv), 3-bromo-2,4-difluoroaniline (179 mg, 0.9 mmol, 1 equiv), Pd(dppf)Cl₂ (63 mg, 0.09 mmol, 0.1 equiv), K₂CO₃ (358 mg, 2.6 mmol, 3 equiv), dioxane (10 mL) and H₂O (2 mL). The resulting solution was stirred for 2 h at 85° C. in an oil bath then cooled and concentrated under vacuum. The residue was purified by silica gel column chromatography, eluting with PE:EA (1:1) to afford 2,4-difluoro-3-[1-(1-[[2-(trimethylsilyl)ethoxy]methyl]imidazol-2-yl)imidazo[1,5-a]pyridin-6-yl]aniline (313 mg, 82% yield) as a yellow solid.

LCMS (ES, m/z): [M+H]⁺: 442

### Synthesis of 18-g: N-[2,4-difluoro-3-[1-(1-[[2-(trimethylsilyl)ethoxy]methyl]imidazol-2-yl)imidazo[1,5-a]pyridin-6-yl]phenyl]-5-fluoro-2-methoxypyridine-3-sulfonamide

Into a 25 mL 3-necked round-bottom flask, was placed 2,4-difluoro-3-[1-(1-[[2-(trimethylsilyl)ethoxy]methyl]imidazol-2-yl)imidazo[1,5-a]pyridin-6-yl]aniline (100 mg, 0.23 mmol, 1 equiv), pyridine (54 mg, 0.68 mmol, 3 equiv) and 5-fluoro-2-methoxypyridine-3-sulfonyl chloride (51 mg, 0.23 mmol, 1 equiv) in DCM (5 mL). The resulting solution was stirred for 2 h at room temperature, then concentrated under vacuum. The residue was purified by silica gel column chromatography, eluting with PE:EA) to afford N-[2,4-difluoro-3-[1-(1-[[2-(trimethylsilyl)ethoxy]methyl]imidazol-2-yl)imidazo[1,5-a]pyridin-6-yl]phenyl]-5-fluoro-2-methoxypyridine-3-sulfonamide (83 mg, 58% yield) as a white solid.

LCMS (ES, m/z): [M+H]⁺: 631

### Synthesis of Compound 18: N-[2,4-difluoro-3-[1-(1H-imidazol-2-yl)imidazo[1,5-a]pyridin-6-yl]phenyl]-5-fluoro-2-methoxypyridine-3-sulfonamide

Into a 25 mL 3-necked round-bottom flask, was placed N-[2,4-difluoro-3-[1-(1-[[2-(trimethylsilyl)ethoxy]methyl]imidazol-2-yl)imidazo[1,5-a]pyridin-6-yl]phenyl]-5-fluoro-2-methoxypyridine-3-sulfonamide (83 mg, 0.13 mmol, 1 equiv) and TFA (3 mL). The resulting solution was stirred for 1 h at 70° C. in an oil bath, then concentrated under vacuum. The crude product was purified by Flash-Prep-HPLC with the following conditions: Column, welch Vltimate XB-C18, 50×250 mm, 10 μm; Mobile Phase 25-40% MeCN over 8 min/0.05% aqueous ammonia; detector, 220 nm. N-[2,4-Difluoro-3-[1-(1H-imidazol-2-yl)imidazo[1,5-a]pyridin-6-yl]phenyl]-5-fluoro-2-methoxypyridine-3-sulfonamide (29 mg, 42% yield) was isolated as a white solid.

LCMS (ES, m/z): [M+H]⁺: 501

1H NMR (300 MHz, DMSO-d6) δ 8.51 (s, 2H), 8.40 (d, J=3.1 Hz, 1H), 8.22 (d, J=9.4 Hz, 1H), 7.99 (dd, J=7.5, 3.1 Hz, 1H), 7.32 (td, J=8.9, 5.8 Hz, 1H), 7.15 (t, J=9.3 Hz, 1H), 7.06 (s, 2H), 6.85 (d, J=9.4 Hz, 1H), 3.89 (s, 3H).

### Example 34: Synthesis of N-[2,4-difluoro-3-[1-(1H-imidazol-2-yl)imidazo[1,5-a]pyridin-6-yl]phenyl]-5-fluoro-2-methylpyridine-3-sulfonamide (Compound 19)

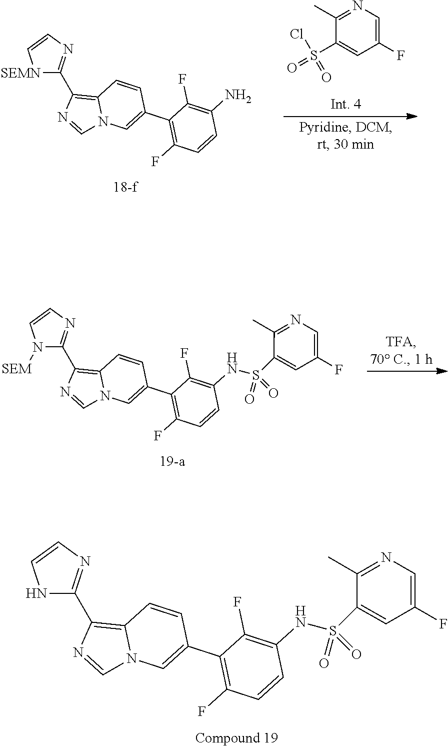 [SMILES: C[Si](C)(C)CCOCn1ccnc1-c1ncn2cc(-c3c(F)ccc(N)c3F)ccc12 ; Cc1ncc(F)cc1S(=O)(=O)Cl ; Cc1ncc(F)cc1S(=O)(=O)Nc1ccc(F)c(-c2ccc3c(-c4nccn4COCC[Si](C)(C)C)ncn3c2)c1F ; Cc1ncc(F)cc1S(=O)(=O)Nc1ccc(F)c(-c2ccc3c(-c4ncc[nH]4)ncn3c2)c1F]

### Synthesis of 19-a: N-[2,4-difluoro-3-[1-(1-[[2-(trimethylsilyl)ethoxy]methyl]imidazol-2-yl)imidazo[1,5-a]pyridin-6-yl]phenyl]-5-fluoro-2-methylpyridine-3-sulfonamide

Into a 25 mL round-bottom flask, was placed 2,4-difluoro-3-[1-(1-[[2-(trimethylsilyl)ethoxy]methyl]imidazol-2-yl)imidazo[1,5-a]pyridin-6-yl]aniline (100 mg, 0.2 mmol, 1 equiv), DCM (10 mL), pyridine (54 mg, 0.7 mmol, 3 equiv) and 5-fluoro-2-methylpyridine-3-sulfonyl chloride (47 mg, 0.2 mmol, 1 equiv). The resulting solution was stirred for 30 min at room temperature, then concentrated under reduced pressure. The residue was purified by silica gel column chromatography, eluting with PE: EA (3:1) to afford N-[2,4-difluoro-3-[1-(1-[[2-(trimethylsilyl)ethoxy]methyl]imidazol-2-yl)imidazo[1,5-a]pyridin-6-yl]phenyl]-5-fluoro-2-methylpyridine-3-sulfonamide (93 mg, 67% yield) as a white solid.

LCMS (ES, m/z): [M+H]⁺: 615

### Synthesis of Compound 19: N-[2,4-difluoro-3-[1-(1H-imidazol-2-yl)imidazo[1,5-a]pyridin-6-yl]phenyl]-5-fluoro-2-methylpyridine-3-sulfonamide

Into a 25 mL round-bottom flask, was placed N-[2,4-difluoro-3-[1-(1-[[2-(trimethylsilyl)ethoxy]methyl]imidazol-2-yl)imidazo[1,5-a]pyridin-6-yl]phenyl]-5-fluoro-2-methylpyridine-3-sulfonamide (93 mg, 0.2 mmol, 1 equiv) and TFA (3 mL). The resulting solution was stirred for 1 h at 70° C. in an oil bath then concentrated under vacuum. The residue was purified by prep-HPLC with the following conditions: Column, welch Vltimate XB-C18, 50×250 mm, 10 μm; Mobile Phase 10-50% MeCN over 15 min/0.1% aqueous formic acid. N-[2,4-Difluoro-3-[1-(1H-imidazol-2-yl)imidazo[1,5-a]pyridin-6-yl]phenyl]-5-fluoro-2-methylpyridine-3-sulfonamide (6.6 mg, 9% yield) was isolated as a white solid.

LCMS (ES, m/z): [M+H]⁺: 485.

¹H NMR (300 MHz, DMSO-d₆) δ 8.72 (d, J=2.9 Hz, 1H), 8.52 (d, J=4.6 Hz, 2H), 8.22 (d, J=9.5 Hz, 1H), 8.00-7.91 (m, 1H), 7.41-7.28 (m, 1H), 7.23 (t, J=9.1 Hz, 1H), 7.10 (s, 2H), 6.82 (d, J=9.4 Hz, 1H), 2.77 (s, 3H).

### Example 35: Synthesis of 5-cyano-N-[2,4-difluoro-3-[1-(1H-imidazol-2-yl)imidazo[1,5-a]pyridin-6-yl]phenyl]-2-methoxypyridine-3-sulfonamide (Compound 20)

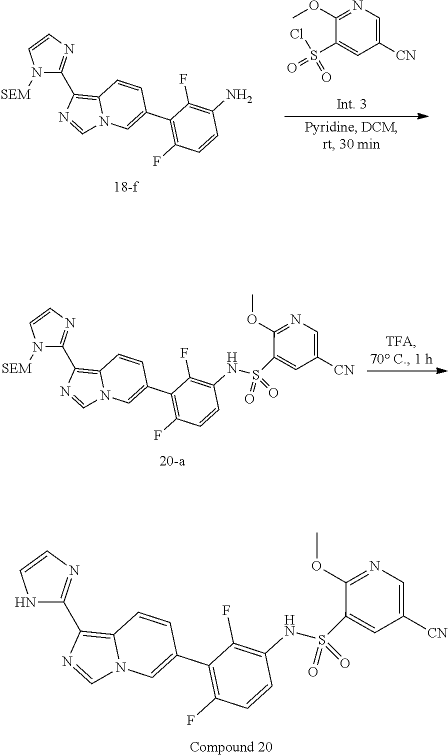 [SMILES: C[Si](C)(C)CCOCn1ccnc1-c1ncn2cc(-c3c(F)ccc(N)c3F)ccc12 ; COc1ncc(C#N)cc1S(=O)(=O)Cl ; COc1ncc(C#N)cc1S(=O)(=O)Nc1ccc(F)c(-c2ccc3c(-c4nccn4COCC[Si](C)(C)C)ncn3c2)c1F ; COc1ncc(C#N)cc1S(=O)(=O)Nc1ccc(F)c(-c2ccc3c(-c4ncc[nH]4)ncn3c2)c1F]

### Synthesis of 20-a: 5-cyano-N-[2,4-difluoro-3-[1-(1-[[2-(trimethylsilyl)ethoxy]methyl]imidazol-2-yl)imidazo[1,5-a]pyridin-6-yl]phenyl]-2-methoxypyridine-3-sulfonamide

Into a 25 mL 3-necked round-bottom flask, was placed 2,4-difluoro-3-[1-(1-[[2-(trimethylsilyl)ethoxy]methyl]imidazol-2-yl)imidazo[1,5-a]pyridin-6-yl]aniline (100 mg, 0.23 mmol, 1 equiv), DCM (5 mL), pyridine (54 mg, 0.68 mmol, 3 equiv) and 5-cyano-2-methoxypyridine-3-sulfonyl chloride (53 mg, 0.23 mmol, 1 equiv). The resulting solution was stirred for 30 min at room temperature then concentrated under vacuum. The residue was purified by silica gel column chromatography, eluting with PE:EA (20:1) to afford 5-cyano-N-[2,4-difluoro-3-[1-(1-[[2-(trimethylsilyl)ethoxy]methyl]imidazol-2-yl)imidazo[1,5-a]pyridin-6-yl]phenyl]-2-methoxypyridine-3-sulfonamide (77 mg, 53% yield) as a white solid.

LCMS (ES, m/z): [M+H]⁺: 638

### Synthesis of Compound 20: 5-cyano-N-[2,4-difluoro-3-[1-(1H-imidazol-2-yl)imidazo[1,5-a]pyridin-6-yl]phenyl]-2-methoxypyridine-3-sulfonamide

Into a 25 mL 3-necked round-bottom flask, was placed 5-cyano-N-[2,4-difluoro-3-[1-(1-[[2-(trimethylsilyl)ethoxy]methyl]imidazol-2-yl)imidazo[1,5-a]pyridin-6-yl]phenyl]-2-methoxypyridine-3-sulfonamide (77 mg, 0.12 mmol, 1 equiv) and TFA (3 mL). The resulting solution was stirred for 60 min at 70° C. in an oil bath, then concentrated under vacuum. The crude product was purified by Flash-Prep-HPLC with the following conditions: welch Vltimate XB-C18, 50×250 mm, 10 μm; Mobile Phase 10-50% MeCN over 15 min/0.1% aqueous formic acid; Detector 220 nm. 5-Cyano-N-[2,4-difluoro-3-[1-(1H-imidazol-2-yl)imidazo[1,5-a]pyridin-6-yl]phenyl]-2-methoxypyridine-3-sulfonamide (24 mg, 37% yield) was isolated as a white solid.

LCMS (ES, m/z): [M+H]⁺: 508

¹H NMR (300 MHz, DMSO-d₆) δ 8.90 (d, J=2.2 Hz, 1H), 8.54-8.44 (m, 3H), 8.22 (d, J=9.4 Hz, 1H), 7.35 (td, J=9.0, 5.9 Hz, 1H), 7.24-7.12 (m, 1H), 7.07 (s, 2H), 6.84 (dd, J=9.6, 1.6 Hz, 1H), 4.00 (s, 3H).

### Example 36: Synthesis of 7-[3-(5-chloro-2-methoxypyridine-3-sulfonamido)-2,6-difluorophenyl]-8-fluoro-N-methylimidazo[1,5-a]pyridine-3-carboxamide (Compound

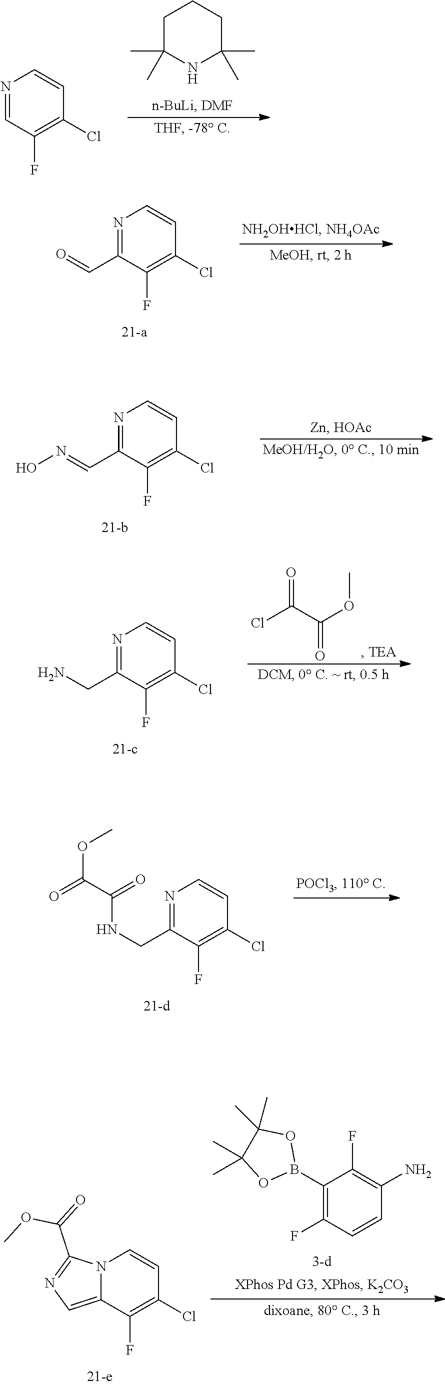

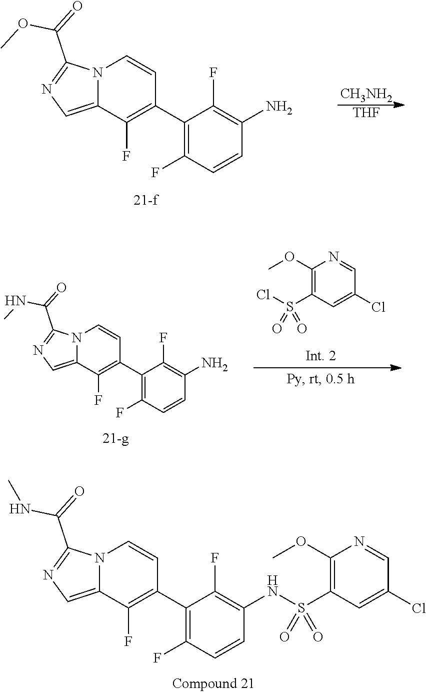 [SMILES: COC(=O)c1ncc2c(F)c(-c3c(F)ccc(N)c3F)ccn12 ; "CH3NH2" ; CNC(=O)c1ncc2c(F)c(-c3c(F)ccc(N)c3F)ccn12 ; COc1ncc(Cl)cc1S(=O)(=O)Cl ; CNC(=O)c1ncc2c(F)c(-c3c(F)ccc(NS(=O)(=O)c4cc(Cl)cnc4OC)c3F)ccn12 ; "THF"]

### Synthesis of 21-a: 4-chloro-3-fluoropicolinaldehyde

To a stirred solution of n-BuLi (38 mL, 92 mmol, 1.2 equiv) in THF (100 mL) was added 2,2,6,6-tetramethylpiperidine (13 g, 92 mmol, 1.2 equiv) dropwise at −78° C. under nitrogen atmosphere. The resulting mixture was stirred for 10 min at −78° C., then 4-chloro-3-fluoropyridine (10 g, 76 mmol, 1 equiv) was added dropwise over 10 min. The resulting mixture was stirred for additional 0.5 h at −78° C., before DMF (6.1 g, 84 mmol, 1.1 equiv) was added dropwise over 10 min. The resulting mixture was stirred for 0.5 h at −78° C. then quenched by the addition of saturated aqueous NaHCO₃ solution (50 mL). The resulting mixture was extracted with EA (3×100 mL) and the combined organics were washed with brine (1×100 mL), then dried over anhydrous sodium sulfate. The solution was concentrated under reduced pressure to give crude 4-chloro-3-fluoropicolinaldehyde (10 g) as a yellow oil, which was used in the next step directly without further purification.

LCMS (ES, m/z): [M+H]⁺: 160

### Synthesis of 21-b: 4-chloro-3-fluoropicolinaldehyde oxime

A solution of 4-chloro-3-fluoropicolinaldehyde (2.7 g, 17 mmol, 1 equiv), NH₂OH·HCl (1.8 g, 25 mmol, 1.5 equiv) and NH₄OAc (3.9 g, 50 mmol, 3 equiv) in methanol (20 mL) was stirred for 2 h at room temperature. The reaction mixture was concentrated under reduced pressure. The residue was diluted in water (30 mL) and extracted with EA (3×50 mL). The combined organics were washed with brine (50 mL), then dried over anhydrous sodium sulfate before being concentrated under reduced pressure. The residue was purified by silica gel column chromatography, eluting with PE/EA (4:1) to afford 4-chloro-3-fluoropicolinaldehydeoxime (2 g, 67% yield) as a yellow solid.

LCMS (ES, m/z): [M+H]⁺: 175

### Synthesis of 21-c: (4-chloro-3-fluoropyridin-2-yl)methanamine

To a stirred solution of 4-chloro-3-fluoropicolinaldehydeoxime (1.2 g, 6.66 mmol, 1 equiv) and HCOOH (2 g, 33.33 mmol, 5 equiv) in MeOH (10 mL) and H₂O (10 mL) was added Zn powder (2.13 g, 33.33 mmol, 5 equiv) in portions at 0° C. The resulting mixture was stirred for 10 min at 0° C., then filtered. The filtrate was concentrated under reduced pressure to afford (4-chloro-3-fluoropyridin-2-yl)methanamine (0.91 g) as a brown oil which was used in the next step directly without further purification.

LCMS (ES, m/z): [M+H]⁺: 161

### Synthesis of 21-d: methyl 2-(((4-chloro-3-fluoropyridin-2-yl)methyl)amino)-2-oxoacetate

To a stirred solution of (4-chloro-3-fluoropyridin-2-yl)methanamine (911 mg, 5.7 mmol, 1 equiv) and TEA (2.88 g, 28.5 mmol, 5 equiv) in DCM (10 mL) was added methyl oxalochloridate (694 mg, 5.7 mmol, 1 equiv) dropwise at 0° C. The resulting mixture was stirred for 0.5 h at room temperature, then concentrated under reduced pressure. The residue was purified by silica gel column chromatography, eluting with PE/EA (10:1) to afford methyl 2-(((4-chloro-3-fluoropyridin-2-yl)methyl)amino)-2-oxoacetate (355 mg, 25% yield) as a yellow oil.

LCMS (ES, m/z): [M+H]+: 247

### Synthesis of 21-e: methyl 7-chloro-8-fluoroimidazo[1,5-a]pyridine-3-carboxylate

A mixture of methyl 2-(((4-chloro-3-fluoropyridin-2-yl)methyl)amino)-2-oxoacetate (350 mg, 1.2 mmol, 1 equiv) and POCl₃ (4 mL) was stirred overnight at 110° C. The reaction mixture was allowed to cool and was concentrated under reduced pressure. The residue was basified to pH 8 with saturated aqueous NaHCO₃ and extracted with EA (3×5 mL). The combined organics were washed with brine (5 mL), dried over anhydrous sodium sulfate and concentrated under reduced pressure. The residue was purified by silica gel column chromatography, eluting with PE/EA (1:1) to afford methyl 7-chloro-8-fluoroimidazo[1,5-a]pyridine-3-carboxylate (105 mg, 32% yield) as a yellow solid.

LCMS (ES, m/z): [M+H]⁺: 229

### Synthesis of 21-f: methyl 7-(3-amino-2,6-difluorophenyl)-8-fluoroimidazo[1,5-a]pyridine-3-carboxylate

To a stirred solution of methyl 7-chloro-8-fluoroimidazo[1,5-a]pyridine-3-carboxylate (100 mg, 0.44 mmol, 1 equiv), K₂CO₃ (121 mg, 0.88 mmol, 2 equiv) and 2,4-difluoro-3-(4,4,5,5-tetramethyl-1,3,2-dioxaborolan-2-yl)aniline (223 mg, 0.88 mmol, 2 equiv) in dioxane (5 mL) were added XPhos (42 mg, 0.088 mmol, 0.2 equiv) and XPhos Pd G3 (37 mg, 0.044 mmol, 0.1 equiv) at room temperature under nitrogen atmosphere. The resulting mixture was stirred for 3 h at 80° C., then was cooled and quenched with water (10 mL). The resulting solution was extracted with EA (3×5 mL). The combined organics were washed with brine (5 mL), dried over anhydrous sodium sulfate and concentrated under reduced pressure. The residue was purified by Flash-Prep-HPLC with the following conditions: Column, WelFlash™ C18-I, Spherical C18 20-40 m, 120 g; mobile phase, 20-70% MeCN over 12 min/0.1% aqueous formic acid; to afford methyl 7-(3-amino-2,6-difluorophenyl)-8-fluoroimidazo[1,5-a]pyridine-3-carboxylate (100 mg, 71% yield) as a yellow oil.

LCMS (ES, m/z): [M+H]⁺: 322

### Synthesis of 21-g: 7-(3-amino-2,6-difluorophenyl)-8-fluoro-N-methylimidazo[1,5-a]pyridine-3-carboxamide

A mixture of methyl 7-(3-amino-2,6-difluorophenyl)-8-fluoroimidazo[1,5-a]pyridine-3-carboxylate (100 mg, 0.31 mmol, 1 equiv), tetrahydrofuran (1 mL) and methylamine solution in water (33%, 4 mL) was stirred for 0.5 h at room temperature. The resulting mixture was extracted with EA (3×5 mL). The combined organics were washed with brine (5 mL), dried over anhydrous sodium sulfate and concentrated under reduced pressure to give 7-(3-amino-2,6-difluorophenyl)-8-fluoro-N-methylimidazo[1,5-a]pyridine-3-carboxamide (90 mg, crude) as a yellow oil which was used in the next step directly without further purification.

LCMS (ES, m/z): [M+H]⁺: 321

### Synthesis of Compound 21: 7-[3-(5-chloro-2-methoxypyridine-3-sulfonamido)-2,6-difluorophenyl]-8-fluoro-N-methylimidazo[1,5-a]pyridine-3-carboxamide

To a stirred solution of 7-(3-amino-2,6-difluorophenyl)-8-fluoro-N-methylimidazo[1,5-a]pyridine-3-carboxamide (90 mg, 0.28 mmol, 1 equiv) in pyridine (5 mL) was added 5-chloro-2-methoxypyridine-3-sulfonyl chloride (102 mg, 0.42 mmol, 1.5 equiv) in portions at room temperature. The resulting mixture was stirred for 0.5 h at room temperature, then concentrated under vacuum. The residue was purified by prep-HPLC with the following conditions: Column, welch Vltimate XB-C18, 50×250 mm, 10 μm; Mobile Phase 10-45% MeCN over 10 min/0.1% aqueous formic acid; to afford 7-[3-(5-chloro-2-methoxypyridine-3-sulfonamido)-2,6-difluorophenyl]-8-fluoro-N-methylimidazo[1,5-a]pyridine-3-carboxamide (40 mg, 27% yield) as a white solid.

LCMS (ES, m/z): [M+H]⁺: 526 H NMR (300 MHz, DMSO-d₆) δ 10.49 (s, 1H), 9.27 (dd, J=7.4, 0.9 Hz, 1H), 8.73 (d, J=4.9 Hz, 1H), 8.48 (s, 1H), 8.07 (d, J=2.7 Hz, 1H), 7.87 (d, J=0.8 Hz, 1H), 7.52-7.38 (m, 1H), 7.25 (s, 1H), 6.94 (t, J=6.9 Hz, 1H), 3.90 (s, 3H), 2.85 (d, J=4.9 Hz, 3H).

### Example 37: Synthesis of 3-[3-(5-chloro-2-methoxypyridine-3-sulfonamido)-2,6-difluorophenyl]-N,1-dimethyl-2-oxoimidazo[1,5-a]pyrimidine-8-carboxamide (Compound 22)

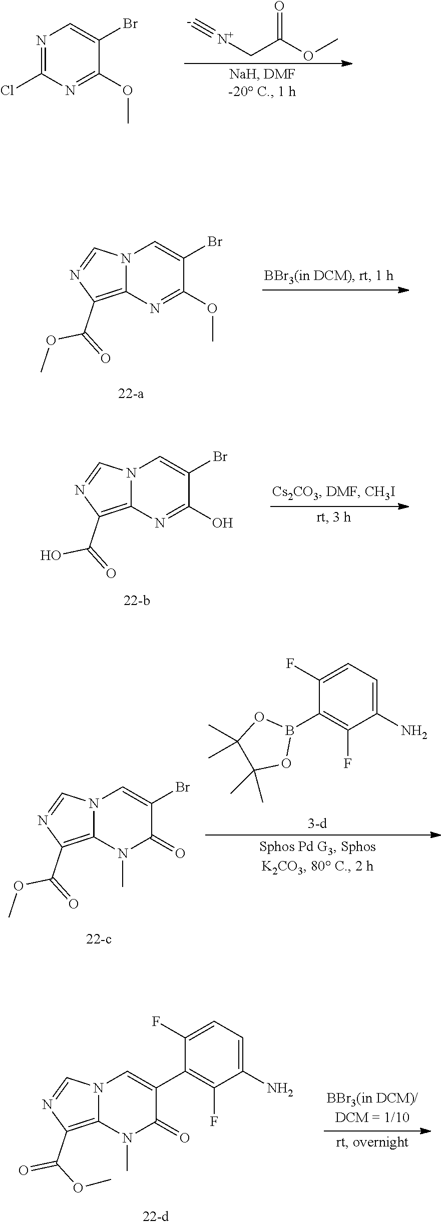

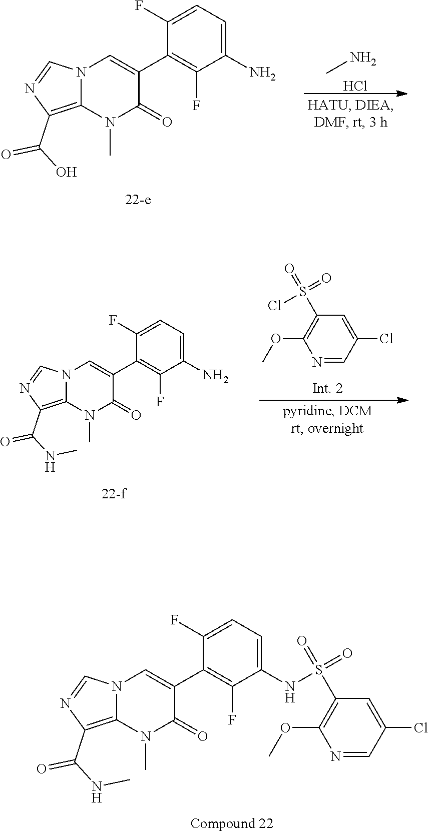

### Synthesis of 22-a: methyl 3-bromo-2-methoxyimidazo[1,5-a]pyrimidine-8-carboxylate

Into a 250 mL 3-necked round-bottom flask, was placed DMF (110 mL). This was followed by the addition of NaH (1.8 g, 44.7 mmol, 2 equiv, 60% in oil), in one portion at −20° C. To this was added methyl 2-isocyanoacetate (3.3 g, 33.6 mmol, 1.5 equiv) dropwise with stirring at −20° C. To the mixture was added a solution of 5-bromo-2-chloro-4-methoxypyrimidine (5 g, 22.4 mmol, 1 equiv) in DMF (30 mL) dropwise with stirring at −20° C. The resulting solution was stirred for 1 h at −20° C., then quenched with 200 mL of water/ice. The solids formed were collected by filtration and air dried to give methyl 3-bromo-2-methoxyimidazo[1,5-a]pyrimidine-8-carboxylate (2.6 g) as a yellow solid which was used in next step directly without further purification.

LCMS (ES, m/z): [M+H]⁺: 286

### Synthesis of 22-b: 3-bromo-2-hydroxyimidazo[1,5-a]pyrimidine-8-carboxylic acid

Into a 40 mL vial, was placed methyl 3-bromo-2-methoxyimidazo[1,5-a]pyrimidine-8-carboxylate (500 mg, 1.75 mmol, 1 equiv), and BBr₃ (8 mL, 1 M in DCM). The resulting solution was stirred for 1 h at 25° C. before being concentrated. The residue was suspended in 10 mL of water, and the solids collected by filtration and air dried, to give 3-bromo-2-hydroxyimidazo[1,5-a]pyrimidine-8-carboxylic acid (300 mg) as a brown solid which was used in next step directly without further purification.

LCMS (ES, m/z): [M+H]⁺: 258

### Synthesis of 22-c: methyl 3-bromo-1-methyl-2-oxoimidazo[1,5-a]pyrimidine-8-carboxylate

To a stirred solution of 3-bromo-2-hydroxyimidazo[1,5-a]pyrimidine-8-carboxylic acid (800 mg, 3.1 mmol, 1 equiv) and Cs₂CO₃ (3 g, 9.3 mmol, 3 equiv) in DMF (32 mL) was added CH₃I (1.3 g, 9.3 mmol, 3 equiv) dropwise at 0° C. The resulting mixture was stirred for 3 h at room temperature. The reaction was quenched with water and the precipitated solids were collected by filtration and washed with water (3×5 mL), before being dried. Methyl 3-bromo-1-methyl-2-oxoimidazo[1,5-a]pyrimidine-8-carboxylate (490 mg) was isolated as a yellow solid which was used in next step directly without further purification.

LCMS (ES, m/z): [M+H]⁺: 286

### Synthesis of 22-d: methyl 3-(3-amino-2,6-difluorophenyl)-1-methyl-2-oxoimidazo[1,5-a]pyrimidine-8-carboxylate

To a stirred mixture of methyl 3-bromo-1-methyl-2-oxoimidazo[1,5-a]pyrimidine-8-carboxylate (490 mg, 1.7 mmol, 1 equiv) and 2,4-difluoro-3-(4,4,5,5-tetramethyl-1,3,2-dioxaborolan-2-yl)aniline (874 mg, 3.4 mmol, 2 equiv) in dioxane (10 mL) and H₂O (2 mL) were added SphosPdGen.3 (134 mg, 0.17 mmol, 0.1 equiv), Sphos (141 mg, 0.34 mmol, 0.2 equiv) and K₂CO₃ (710 mg, 5.1 mmol, 3 equiv) at room temperature under N₂ atmosphere.

The resulting mixture was stirred for 2 h at 80° C. under N₂ atmosphere. The mixture was allowed to cool and was concentrated under reduced pressure. The residue was purified by silica gel column chromatography, eluting with PE/EA (1/1) to afford methyl 3-(3-amino-2,6-difluorophenyl)-1-methyl-2-oxoimidazo[1,5-a]pyrimidine-8-carboxylate (300 mg) as a yellow solid.

LCMS (ES, m/z): [M+H]⁺: 335

### Synthesis of 22-e: 3-(3-amino-2,6-difluorophenyl)-1-methyl-2-oxoimidazo[1,5-a]pyrimidine-8-carboxylic acid

To a stirred solution of methyl 3-(3-amino-2,6-difluorophenyl)-1-methyl-2-oxoimidazo[1,5-a]pyrimidine-8-carboxylate (190 mg, 0.6 mmol, 1 equiv) in DCM (6 mL) was added BBr₃ (1.2 mL, 1 M in DCM) at room temperature. The resulting mixture was stirred overnight at room temperature, then concentrated under vacuum. The product was precipitated by the addition of water (5 mL) and this was collected by filtration and washed with water (2×5 mL). Drying afforded 3-(3-amino-2,6-difluorophenyl)-1-methyl-2-oxoimidazo[1,5-a]pyrimidine-8-carboxylic acid (110 mg) as a yellow solid which was used in next step directly without further purification.

LCMS (ES, m/z): [M+H]⁺: 321

### Synthesis of 22-f: 3-(3-amino-2,6-difluorophenyl)-N,1-dimethyl-2-oxoimidazo[1,5-a]pyrimidine-8-carboxamide

Into an 8 mL vial, was placed 3-(3-amino-2,6-difluorophenyl)-1-methyl-2-oxoimidazo[1,5-a]pyrimidine-8-carboxylic acid (100 mg, 0.3 mmol, 1 equiv), DMF (4 mL), HATU (178 mg, 0.5 mmol, 1.5 equiv), DIEA (121 mg, 0.9 mmol, 3 equiv) and methylamine hydrochloride (42 mg, 0.6 mmol, 2 equiv). The resulting solution was stirred for 3 h at 25° C., then concentrated. The crude product was purified by Flash-Prep-HPLC with the following conditions: Column, welch Vltimate XB-C18, 50×250 mm, 10 μm; mobile phase, 5-60% MeCN over 20 mins/0.1% aqueous formic acid; Detector, 254 nm. 3-(3-Amino-2,6-difluorophenyl)-N,1-dimethyl-2-oxoimidazo[1,5-a]pyrimidine-8-carboxamide (60 mg) was isolated as a yellow solid.

LCMS (ES, m/z): [M+H]⁺:334

### Synthesis of Compound 22: 3-[3-(5-chloro-2-methoxypyridine-3-sulfonamido)-2,6-difluorophenyl]-N,1-dimethyl-2-oxoimidazo[1,5-a]pyrimidine-8-carboxamide

Into an 8 mL vial, was placed 3-(3-amino-2,6-difluorophenyl)-N,1-dimethyl-2-oxoimidazo[1,5-a]pyrimidine-8-carboxamide (30 mg, 0.1 mmol, 1 equiv), DCM (2 mL), pyridine (36 mg, 0.45 mmol, 5 equiv) and 5-chloro-2-methoxypyridine-3-sulfonyl chloride (26 mg, 0.1 mmol, 1.2 equiv). The resulting solution was stirred overnight at 25° C., then concentrated. The crude product was purified by Prep-HPLC with the following conditions: Column, welch Vltimate XB-C18, 50×250 mm, 10 μm; mobile phase, 33-53% MeCN over 8 mins/0.1% aqueous formic acid; Detector, 254 nm. 3-[3-(5-Chloro-2-methoxypyridine-3-sulfonamido)-2,6-difluorophenyl]-N,1-dimethyl-2-oxoimidazo[1,5-a]pyrimidine-8-carboxamide (15.6 mg) was isolated as an off-white solid.

LCMS (ES, m/z): [M+H]⁺: 539

¹H NMR (300 MHz, DMSO-d₆) δ 10.48 (s, 1H), 8.65 (s, 1H), 8.48 (d, J=2.6 Hz, 1H), 8.22-8.11 (m, 1H), 8.07 (d, J=2.6 Hz, 1H), 8.00 (s, 1H), 7.37 (td, J=8.9, 5.9 Hz, 1H), 7.16 (t, J=8.9 Hz, 1H), 3.88 (d, J=9.3 Hz, 6H), 2.77 (d, J=4.8 Hz, 3H).

### Example 38: Synthesis of 6-[3-[3-cyano-5-(trifluoromethyl)benzenesulfonamido]-2,6-difluorophenyl]-N-methylimidazo[1,5-a]pyridine-1-carboxamide (Compound 23)

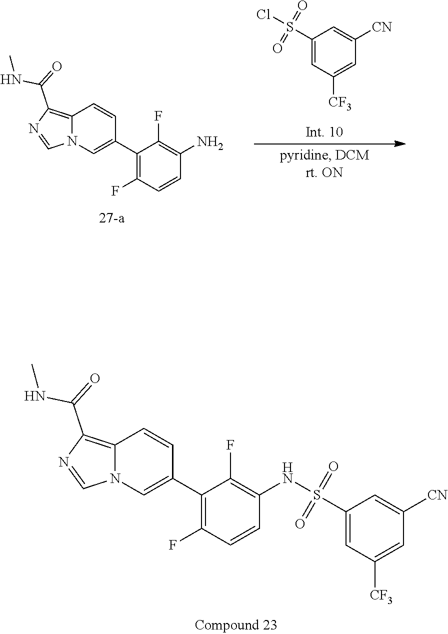 [SMILES: CNC(=O)c1ncn2cc(-c3c(F)ccc(N)c3F)ccc12 ; N#Cc1cc(C(F)(F)F)cc(S(=O)(=O)Cl)c1 ; CNC(=O)c1ncn2cc(-c3c(F)ccc(NS(=O)(=O)c4cc(C#N)cc(C(F)(F)F)c4)c3F)ccc12]

### Synthesis of Compound 23: 6-[3-[3-cyano-5-(trifluoromethyl)benzenesulfonamido]-2,6-difluorophenyl]-N-methylimidazo[1,5-a]pyridine-1-carboxamide

To a stirred solution of 6-(3-amino-2,6-difluorophenyl)-N-methylimidazo[1,5-a]pyridine-1-carboxamide (100 mg, 0.33 mmol, 1 equiv) and 3-cyano-5-(trifluoromethyl)benzenesulfonyl chloride (178 mg, 0.66 mmol, 2 equiv) in DCM (5 ml) was added pyridine (52 mg, 0.66 mmol, 2 equiv) at 0° C., and the reaction was stirred at room temperature overnight. The reaction was concentrated and the residue purified by prep-HPLC using the following conditions: Column, welch Vltimate XB-C18, 50×250 mm, 10 μm; Mobile Phase 20-60% MeCN over 15 min/0.1% aqueous formic acid; to give 6-[3-[3-cyano-5-(trifluoromethyl)benzenesulfonamido]-2,6-difluorophenyl]-N-methylimidazo[1,5-a]pyridine-1-carboxamide (30 mg, 17% yield) as a white solid.

LCMS (ES, m/z): [M+H]⁺: 536

¹H NMR (300 MHz, DMSO-d₆) δ 10.69 (s, 1H), 8.72 (s, 1H), 8.58 (s, 1H), 8.49 (d, J=4.5 Hz, 2H), 8.28 (s, 1H), 8.15-8.08 (m, 2H), 7.34-7.22 (m, 2H), 6.97 (d, J=9.4 Hz, 1H), 2.81 (d, J=4.3 Hz, 3H).

### Example 39: Synthesis of 6-[3-(5-chloro-2-methoxypyridine-3-sulfonamido)-2,6-difluorophenyl]-N-methyl-5-(methylamino)imidazo[1,5-a]pyridine-1-carboxamide (Compound 24)

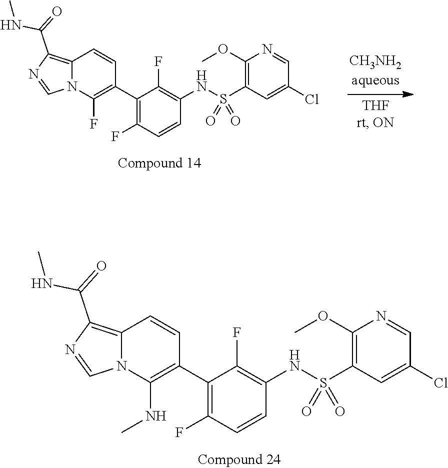 [SMILES: CNC(=O)c1ncn2c(F)c(-c3c(F)ccc(NS(=O)(=O)c4cc(Cl)cnc4OC)c3F)ccc12 ; CNC(=O)c1ncn2c(NC)c(-c3c(F)ccc(NS(=O)(=O)c4cc(Cl)cnc4OC)c3F)ccc12]

### Synthesis of Compound 24: 6-[3-(5-chloro-2-methoxypyridine-3-sulfonamido)-2,6-difluorophenyl]-N-methyl-5-(methylamino)imidazo[1,5-a]pyridine-1-carboxamide

To a stirred solution of 6-[3-(5-chloro-2-methoxypyridine-3-sulfonamido)-2,6-difluorophenyl]-5-fluoro-N-methylimidazo[1,5-a]pyridine-1-carboxamide (80 mg, 0.15 mmol, 1 equiv) in THE (1 mL) was added 40% aqueous CH₃NH₂ (14 mg, 0.45 mmol, 3 equiv), and the reaction was stirred at room temperature overnight. The solution was concentrated directly and the residue was purified by prep-HPLC using the following conditions: Column, welch Vltimate XB-C18, 50×250 mm, 10 μm; Mobile Phase 30-70% MeCN over 15 mins/0.1% aqueous formic acid; Detector, 220 nm. 6-[3-(5-chloro-2-methoxypyridine-3-sulfonamido)-2,6-difluorophenyl]-N-methyl-5-(methylamino)imidazo[1,5-a]pyridine-1-carboxamide (40 mg, 49% yield) was isolated as a white solid.

LCMS (ES, m/z): [M+H]⁺: 537

1H NMR (300 MHz, Methanol-d4) δ 8.41-8.32 (m, 2H), 8.03 (d, J=2.6 Hz, 1H), 7.64 (dd, J=9.2, 0.8 Hz, 1H), 7.55 (td, J=8.9, 5.8 Hz, 1H), 7.08 (td, J=8.8, 1.9 Hz, 1H), 6.79 (d, J=9.2 Hz, 1H), 4.07 (s, 3H), 2.98 (s, 3H), 2.37 (s, 3H).

### Example 40: Synthesis of 5-chloro-N-[2,4-difluoro-3-[1-(1H-imidazol-2-yl)imidazo[1,5-a]pyridin-6-yl]phenyl]-2-methylpyridine-3-sulfonamide (Compound 25)

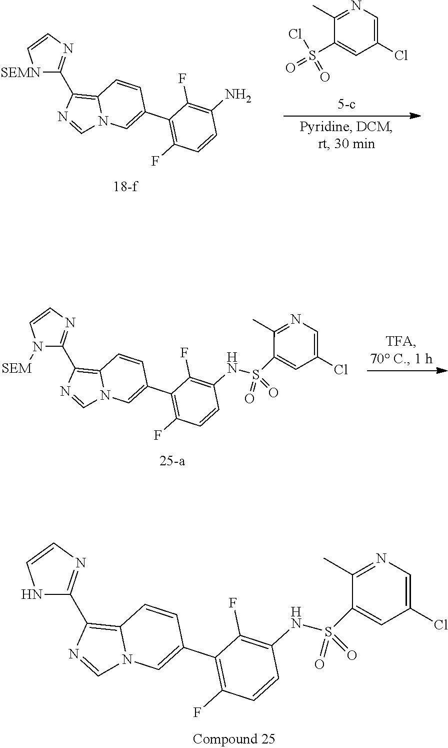 [SMILES: C[Si](C)(C)CCOCn1ccnc1-c1ncn2cc(-c3c(F)ccc(N)c3F)ccc12 ; Cc1ncc(Cl)cc1S(=O)(=O)Cl ; Cc1ncc(Cl)cc1S(=O)(=O)Nc1ccc(F)c(-c2ccc3c(-c4nccn4COCC[Si](C)(C)C)ncn3c2)c1F ; Cc1ncc(Cl)cc1S(=O)(=O)Nc1ccc(F)c(-c2ccc3c(-c4ncc[nH]4)ncn3c2)c1F]

### Synthesis of 25-a: 5-chloro-N-[2,4-difluoro-3-[1-(1-[[2-(trimethylsilyl)ethoxy]methyl]imidazol-2-yl)imidazo[1,5-a]pyridin-6-yl]phenyl]-2-methylpyridine-3-sulfonamide

Into a 25 mL 3-necked round-bottom flask, was placed DCM (5 mL), 2,4-difluoro-3-[1-(1-[[2-trimethylsilyl)ethoxy]methyl]imidazol-2-yl)imidazo[1,5-a]pyridin-6-yl]aniline (100 Mg, 0.2 mmol, 1 equiv), pyridine (54 mg, 0.6 mmol, 3 equiv) and 5-chloro-2-methylpyridine-3-sulfonyl chloride (51 ng, 0.2 mmol, 1 equiv), The resulting solution was stirred for 2 h at room temperature. The mixture was concentrated under reduced pressure and the residue purified by silica gel column chromatography, eluting with PE:EA (3:2) to afford 5-chloro-N-[2,4-difluoro-3-[1-(1-[[2-(trimethylsilyl)ethoxy]methyl]imidazol-2-yl)imidazo[1,5-a]pyridin-6-yl]phenyl]-2-methylpyridine-3-sulfonamide (83 mg, 58% yield) as yellow oil.

LCMS (ES, m/z): [M+H]^{m}: 631

### Synthesis of Compound 25: 5-chloro-N-[2,4-difluoro-3-[1-(1H-imidazol-2-yl)imidazo[1,5-a]pyridin-6-yl]phenyl]-2-methylpyridine-3-sulfonamide

Into a 25 mL 3-necked round-bottom flask, was placed 5-chloro-N-[2,4-difluoro-3-[1-(1-[[2-(trimethylsilyl)ethoxy]methyl]imidazol-2-yl)imidazo[1,5-a]pyridin-6-yl]phenyl]-2-methylpyridine-3-sulfonamide (83 mg, 0.1 mmol, 1 equiv) and TFA (3 mL). The resulting solution was stirred for 1 h at 70° C. in an oil bath, then cooled and concentrated. The residue was purified by prep-HPLC with the following conditions Column, welch Vltimate XB-C18, 50×250 mm, 10 μm; Mobile Phase 5-40% MeCN over 15 min/0.1% aqueous formic acid; 5-Chloro-N-[2,4-difluoro-3-[1-(1H-imidazol-2-yl)imidazo[1,5-a]pyridin-6-yl]phenyl]-2-methylpyridine-3-sulfonamide (15 mg, 21% yield) was isolated as a yellow solid.

LCMS (ES, m/z): [M+H]⁺: 501

¹H NMR (300 MHz, Methanol-d₄) δ 8.65 (d, J=2.4 Hz, 1H), 8.46 (d, J=11.3 Hz, 2H), 8.20-8.09 (m, 2H), 7.54 (td, J=8.9, 5.6 Hz, 1H), 7.32 (s, 2H), 7.24-7.11 (m, 1H), 6.98 (d, J=9.4 Hz, 1H), 2.85 (s, 3H).

### Example 41: Synthesis of 5-chloro-N-[3,5-difluoro-4-[1-(1H-imidazol-2-yl)imidazo[1,5-a]pyridin-6-yl]pyridin-2-yl]-2-methoxypyridine-3-sulfonamide (Compound 26)

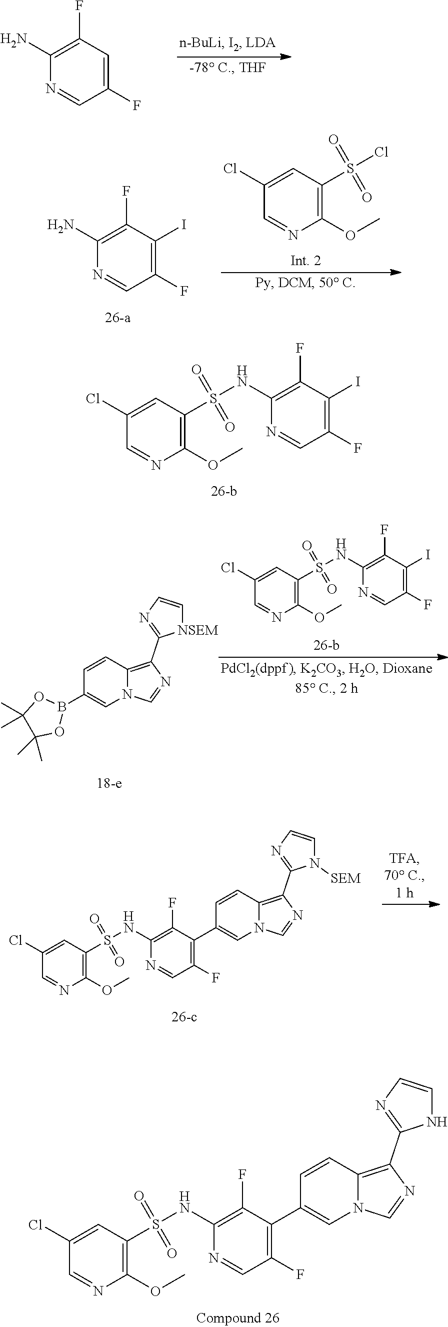

### Synthesis of 26-a: 3,5-difluoro-4-iodopyridin-2-amine

To a stirred solution of 3,5-difluoropyridin-2-amine (5 g, 38 mmol, 1 equiv) in THE (200 mL) was added LDA (61 mL, 123 mmol, 3 equiv) dropwise at −78° C. under N₂ atmosphere. The solution was stirred for 1.5 h at −78° C. To the resulting mixture was added a solution of I₂ (34 g, 135 mmol, 3.5 equiv) in THE (50 mL) dropwise at −78° C. This mixture was stirred for additional 0.5 h at −78° C., then was quenched with saturated aqueous Na₂S₂O₃ (100 mL). The reaction was extracted with EA (3×50 mL), and the combined organics were washed with brine (2×50 mL) and dried over anhydrous sodium sulfate before being concentrated. The residue was purified by silica gel column chromatography, eluting with PE:EA (3:1) to afford 3,5-difluoro-4-iodopyridin-2-amine (7.8 g, 79% yield) as a yellow solid.

LCMS (ES, m/z): [M+H]⁺: 257

### Synthesis of 26-b: 5-chloro-N-(3,5-difluoro-4-iodopyridin-2-yl)-2-methoxypyridine-3-sulfonamide

Into a 50 mL round-bottom flask was placed 3,5-difluoro-4-iodopyridin-2-amine (100 mg, 0.4 mmol, 1 equiv), pyridine (2 mL) and 5-chloro-2-methoxypyridine-3-sulfonyl chloride (142 mg, 0.6 mmol, 1.5 equiv). The resulting solution was stirred for 12 h at 50° C. in an oil bath. The resulting mixture was washed with 3×6 mL of H₂O, then was extracted with 2×6 mL of dichloromethane. The combined organics were dried over anhydrous sodium sulfate and concentrated under vacuum. The residue was purified by silica gel column chromatography, eluting with PE:EA (3:1) to afford 5-chloro-N-(3,5-difluoro-4-iodopyridin-2-yl)-2-methoxypyridine-3-sulfonamide (90 mg, 50% yield) as light yellow oil.

LCMS (ES, m/z): [M+H]⁺: 462

### Synthesis of 26-c: 5-chloro-N-[3,5-difluoro-4-[1-(1-[[2-(trimethylsilyl)ethoxy]methyl]imidazol-2-yl)imidazo[1,5-a]pyridin-6-yl]pyridin-2-yl-2-methoxypyridine-3-sulfonamide

Into a 50 mL round-bottom flask was placed 2-[6-(4,4,5,5-tetramethyl-1,3,2-dioxaborolan-2-yl)imidazo[1,5-a]pyridin-1-yl]-1-[[2-(trimethylsilyl)ethoxy]methyl]imidazole (45 mg, 0.1 mmol, 1 equiv), dioxane (2 mL), 5-chloro-N-(3,5-difluoro-4-iodopyridin-2-yl)-2-methoxypyridine-3-sulfonamide (47 mg, 0.1 mmol, 1 equiv), Pd(dppf)Cl₂ (5 mg, 0.007 mmol, 0.07 equiv), K₂CO₃ (42 mg, 0.3 mmol, 3 equiv) and H₂O (0.5 mL). The resulting solution was stirred for 2 h at 85° C. in an oil bath. The resulting mixture was washed with 2×6 mL of H₂O, then extracted with 2×6 mL of dichloromethane. The combined organics were dried over anhydrous sodium sulfate and concentrated under vacuum. The residue was purified by silica gel column chromatography, eluting with PE:EA (3:1) to afford 5-chloro-N-[3,5-difluoro-4-[1-(1-[[2-(trimethylsilyl)ethoxy]methyl]imidazol-2-yl)imidazo[1,5-a]pyridin-6-yl]pyridin-2-yl]-2-methoxypyridine-3-sulfonamide (60 mg, 91% yield) as a light yellow oil.

LCMS (ES, m/z): [M+H]⁺: 648

### Synthesis of Compound 26: 5-chloro-N-[3,5-difluoro-4-[1-(1H-imidazol-2-yl)imidazo[1,5-a]pyridin-6-yl]pyridin-2-yl]-2-methoxypyridine-3-sulfonamide

Into a 50 mL round-bottom flask was placed 5-chloro-N-[3,5-difluoro-4-[1-(1-[[2-(trimethylsilyl)ethoxy]methyl]imidazol-2-yl)imidazo[1,5-a]pyridin-6-yl]pyridin-2-yl]-2-methoxypyridine-3-sulfonamide (50 mg, 0.1 mmol, 1 equiv) and TFA (2 mL). The resulting solution was stirred for 1 h at 70° C. in an oil bath, then concentrated under vacuum. The residue was purified by prep-HPLC with the following conditions: Column, welch Vltimate XB-C18, 50×250 mm, 10 μm; Mobile Phase 5-35% MeCN over 15 mins/0.1% aqueous formic acid; to afford 5-chloro-N-[3,5-difluoro-4-[1-(1H-imidazol-2-yl)imidazo[1,5-a]pyridin-6-yl]pyridin-2-yl]-2-methoxypyridine-3-sulfonamide (3 mg, 6.8% yield) as a white solid.

LCMS (ES, m/z): [M+H]⁺: 518

¹H NMR (300 MHz, Methanol-d₄) δ 8.80 (s, 1H), 8.65 (s, 1H), 8.44-8.36 (m, 1H), 8.35-8.28 (m, 1H), 8.24-8.11 (m, 2H), 7.58 (s, 2H), 7.40 (d, J=9.3 Hz, 1H), 4.06 (d, J=1.3 Hz, 3H).

### Example 42: Synthesis of (6R)-6-[3-(5-chloro-2-methoxypyridine-3-sulfonamido)-2,6-difluorophenyl]-N-methyl-5H,6H,7H,8H-imidazo[1,5-a]pyridine-1-carboxamide & (6S)-6-[3-(5-chloro-2-methoxypyridine-3-sulfonamido)-2,6-difluorophenyl]-N-methyl-5H,6H,7H,8H-imidazo[1,5-a]pyridine-1-carboxamide (Compound 27-1 and 27-2)

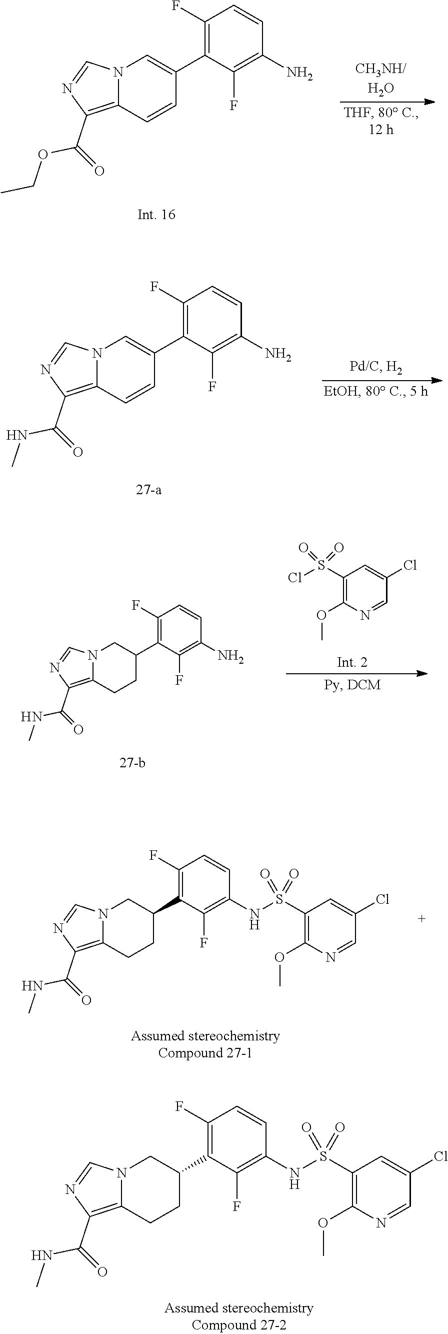 [SMILES: CCOC(=O)c1ncn2cc(-c3c(F)ccc(N)c3F)ccc12 ; CNC(=O)c1ncn2cc(-c3c(F)ccc(N)c3F)ccc12 ; CNC(=O)c1ncn2c1CCC(c1c(F)ccc(N)c1F)C2 ; COc1ncc(Cl)cc1S(=O)(=O)Cl ; CNC(=O)c1ncn2c1CC[C@H](c1c(F)ccc(NS(=O)(=O)c3cc(Cl)cnc3OC)c1F)C2 ; CNC(=O)c1ncn2c1CC[C@@H](c1c(F)ccc(NS(=O)(=O)c3cc(Cl)cnc3OC)c1F)C2]

### Synthesis of 27-a: 6-(3-amino-2,6-difluorophenyl)-N-methylimidazo[1,5-a]pyridine-1-carboxamide

To a stirred mixture of ethyl 6-(3-amino-2,6-difluorophenyl)imidazo[1,5-a]pyridine-1-carboxylate (2 g, 6.3 mmol, 1 equiv) in THF (10 mL) was added 40% methylamine solution in water (20 mL) at room temperature. The resulting solution was stirred for 12 hours at 80° C. The mixture was allowed to cool to room temperature and concentrated under reduced pressure. The residue was purified by Flash-Prep-HPLC with the following conditions: Column, WelFlash™ C18-I, Spherical C18 20-40 m, 120 g; mobile phase: 20-70% MeCN/0.1% NH₄HCO₃ and 0.05% NH₃·H₂O; to give 6-(3-amino-2,6-difluorophenyl)-N-methylimidazo[1,5-a]pyridine-1-carboxamide (1.6 g, 83% yield) as a white solid.

LCMS (ES, m/z): [M+H]⁺: 303

### Synthesis of 27-b: 6-(3-amino-2,6-difluorophenyl)-N-methyl-5H,6H,7H,8H-imidazo[1,5-a]pyridine-1-carboxamide

To a solution of 6-(3-amino-2,6-difluorophenyl)-N-methylimidazo[1,5-a]pyridine-1-carboxamide (750 mg, 2.5 mmol, 1 equiv) in EtOH (25 mL) was added Pd/C (10%, 0.8 g) in a pressure tank. The mixture was hydrogenated at 80° C. under 20 atm of hydrogen pressure for 5 hours, then filtered through a Celite pad and concentrated under reduced pressure. The residue was purified by Flash-Prep-HPLC with the following conditions: Column, WelFlash™ C18-I, Spherical C18 20-40 m, 120 g, mobile phase: 20-70% MeCN/0.1% NH₄HCO₃ & 0.05% NH₃·H₂O; to give 6-(3-amino-2,6-difluorophenyl)-N-methyl-5H,6H,7H,8H-imidazo[1,5-a]pyridine-1-carboxamide (280 mg, 36% yield) as a white solid.

LCMS (ES, m/z): [M+H]⁺: 307

¹H NMR (300 MHz, DMSO-d₆) δ 7.75 (d, J=5.0 Hz, 1H), 7.56 (s, 1H), 6.81 (ddd, J=10.3, 8.9, 1.4 Hz, 1H), 6.70 (td, J=9.3, 5.7 Hz, 1H), 5.03 (s, 2H), 4.32 (dd, J=12.2, 5.3 Hz, 1H), 4.08 (t, J=12.0 Hz, 1H), 3.44-3.27 (m, 1H), 3.32 (s, 1H), 2.88 (ddd, J=17.8, 11.8, 6.2 Hz, 1H), 2.72 (d, J=4.7 Hz, 3H), 2.19 (s, 1H), 2.05 (s, 1H).

### Synthesis of Compounds 27-1 and 27-2: (6R)-6-[3-(5-chloro-2-methoxypyridine-3-sulfonamido)-2,6-difluorophenyl]-N-methyl-5H,6H,7H,8H-imidazo[1,5-a]pyridine-1-carboxamide & (6S)-6-[3-(5-chloro-2-methoxypyridine-3-sulfonamido)-2,6-difluorophenyl]-N-methyl-5H,6H,7H,8H-imidazo[1,5-a]pyridine-1-carboxamide

To a stirred solution of 6-(3-amino-2,6-difluorophenyl)-N-methyl-5H,6H,7H,8H-imidazo[1,5-a]pyridine-1-carboxamide (200 mg, 0.65 mmol, 1 equiv) and pyridine (310 mg, 3.92 mmol, 6 equiv) in DCM (6 mL) was added a solution of 5-chloro-2-methoxypyridine-3-sulfonyl chloride (238 mg, 0.98 mmol, 1.5 equiv) in DCM (2 mL) dropwise at 0° C. The resulting solution was stirred for 2.5 hours, then concentrated under reduced pressure. The residue was purified by prep-HPLC with the following conditions: Column, welch Vltimate XB-C18, 50×250 mm, 10 μm mobile phase, Mobile Phase A: 0.1% FA in Water, Mobile Phase B: MeCN (10% up to 30% in 10 min) and chiral separation with the following conditions: Column, CHIRALPAK IF, 20×250 mm, 5 μm, Mobile Phase A: Hexane: DCM (3:1). Mobile phase B: EtOH (10% up to 30% in 15 min) to give (6R)-6-[3-(5-chloro-2-methoxypyridine-3-sulfonamido)-2,6-difluorophenyl]-N-methyl-5H,6H,7H,8H-imidazo[1,5-a]pyridine-1-carboxamide (25 mg, 7.5% yield) as a white solid and (6S)-6-[3-(5-chloro-2-methoxypyridine-3-sulfonamido)-2,6-difluorophenyl]-N-methyl-5H,6H,7H,8H-imidazo[1,5-a]pyridine-1-carboxamide (21 mg, 6% yield) as a white solid.

Stereochemistry was randomly assigned.

Compound 27-1

LCMS (ES, m/z): [M+H]⁺: 512

¹H NMR (300 MHz, DMSO-d₆) δ 10.33 (s, 1H), 8.52 (d, J=2.6 Hz, 1H), 8.02 (d, J=2.6 Hz, 1H), 7.74 (d, J=5.0 Hz, 1H), 7.55 (s, 1H), 7.26 (td, J=8.8, 5.7 Hz, 1H), 7.11 (t, J=9.5 Hz, 1H), 4.26 (dd, J=12.3, 5.2 Hz, 1H), 4.00 (d, J=11.9 Hz, 1H), 3.92 (s, 3H), 3.49 (s, 1H), 2.87 (ddd, J=17.8, 11.6, 6.3 Hz, 1H), 2.72 (d, J=4.8 Hz, 3H), 2.07 (d, J=9.0 Hz, 1H), 1.97 (s, 2H).

Compound 27-2

LCMS (ES, m/z): [M+H]⁺: 512

¹H NMR (300 MHz, DMSO-d₆) δ 10.33 (s, 1H), 8.52 (d, J=2.6 Hz, 1H), 8.02 (d, J=2.6 Hz, 1H), 7.74 (d, J=5.0 Hz, 1H), 7.55 (s, 1H), 7.33-7.19 (m, 1H), 7.11 (t, J=9.5 Hz, 1H), 4.26 (dd, J=12.3, 5.2 Hz, 1H), 4.00 (d, J=11.9 Hz, 1H), 3.93 (s, 3H), 3.49 (s, 1H), 2.87 (ddd, J=17.9, 11.8, 6.3 Hz, 1H), 2.72 (d, J=4.7 Hz, 3H), 2.07 (d, J=12.3 Hz, 1H), 1.97 (s, 2H).

### Example 43: Synthesis of N-[2,4-difluoro-3-[1-(1H-imidazol-2-yl)imidazo[1,5-a]pyridin-6-yl]phenyl]-6-fluoro-1-hydroxy-2,3-dihydro-1H-indene-4-sulfonamide (Compound 28)

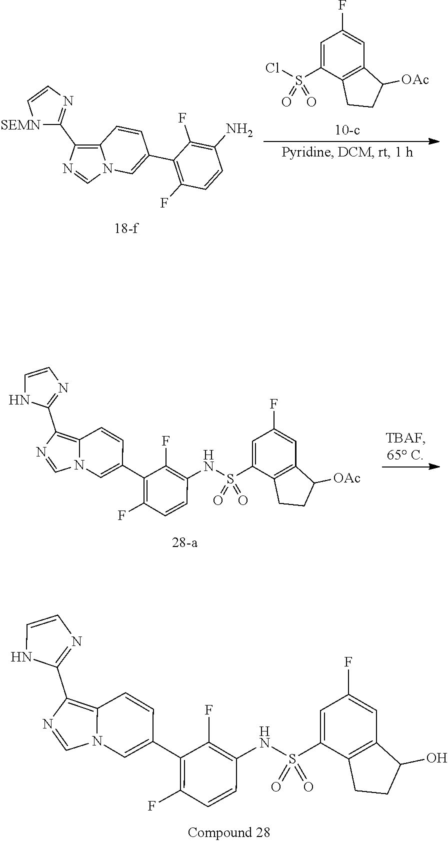 [SMILES: C[Si](C)(C)CCOCn1ccnc1-c1ncn2cc(-c3c(F)ccc(N)c3F)ccc12 ; CC(=O)OC1CCc2c1cc(F)cc2S(=O)(=O)Cl ; CC(=O)OC1CCc2c1cc(F)cc2S(=O)(=O)Nc1ccc(F)c(-c2ccc3c(-c4ncc[nH]4)ncn3c2)c1F ; O=S(=O)(Nc1ccc(F)c(-c2ccc3c(-c4ncc[nH]4)ncn3c2)c1F)c1cc(F)cc2c1CCC2O]

### Synthesis of 28-a: 4-([2,4-difluoro-3-[1-(1H-imidazol-2-yl)imidazo[1,5-a]pyridin-6-yl]phenyl]sulfamoyl)-6-fluoro-2,3-dihydro-1H-inden-1-yl acetate

Into a 50 mL round-bottom flask was placed 2,4-difluoro-3-[1-(1-[[2-(trimethylsilyl)ethoxy]methyl]imidazol-2-yl)imidazo[1,5-a]pyridin-6-yl]aniline (90 mg, 0.2 mmol, 1 equiv), pyridine (4 mL) and 4-(chlorosulfonyl)-6-fluoro-2,3-dihydro-1H-inden-1-yl acetate (71 mg, 0.2 mmol, 1 equiv). The resulting solution was stirred for 1 h at room temperature. The resulting mixture was diluted with H₂O (10 mL) and was extracted with dichloromethane (2×6 mL). The combined organics were washed with H₂O (2×6 mL), then dried over anhydrous sodium sulfate. Concentration gave the crude product, which was purified by silica gel column chromatography, eluting with PE:EA (3:2) to afford 4-([2,4-difluoro-3-[1-(1H-imidazol-2-yl)imidazo[1,5-a]pyridin-6-yl]phenyl]sulfamoyl)-6-fluoro-2,3-dihydro-1H-inden-1-yl acetate (80 mg, 69% yield) as a yellow oil.

LCMS (ES, m/z): [M+H]⁺: 568.

### Synthesis of Compound 28: N-[2,4-difluoro-3-[1-(1H-imidazol-2-yl)imidazo[1,5-a]pyridin-6-yl]phenyl]-6-fluoro-1-hydroxy-2,3-dihydro-1H-indene-4-sulfonamide

Into a 50 mL round-bottom flask was placed 4-([2,4-difluoro-3-[1-(1-[[2-(trimethylsilyl)ethoxy]methyl]imidazol-2-yl)imidazo[1,5-a]pyridin-6-yl]phenyl]sulfamoyl)-6-fluoro-2,3-dihydro-1H-inden-1-yl acetate (70 mg, 0.1 mmol, 1 equiv) and TBAF (1 mol/L in THF, 1 mL). The resulting solution was stirred for 12 h at 65° C. in an oil bath, then cooled and diluted with H₂O (10 mL). The resulting mixture was extracted with dichloromethane (2×10 mL). The combined organics were washed with H₂O (2×6 mL), then dried over anhydrous sodium sulfate. Concentration gave the crude product which was purified by prep-HPLC with the following conditions: Column, welch Vltimate XB-C18, 50×250 mm, 10 μm; Mobile Phase 20-40% MeCN/0.1% aqueous formic acid; to afford N-[2,4-difluoro-3-[1-(1H-imidazol-2-yl)imidazo[1,5-a]pyridin-6-yl]phenyl]-6-fluoro-1-hydroxy-2,3-dihydro-1H-indene-4-sulfonamide (5.5 mg, 10% yield) as a light yellow solid.

LCMS (ES, m/z): [M+H]⁺: 526.

¹H NMR (300 MHz, DMSO-d₆) δ 12.37 (br s, 1H), 8.48 (m, 2H), 8.25-8.13 (m, 2H), 7.34 (d, J=8.8 Hz, 2H), 7.30-7.19 (m, 1H), 7.0-7.14 (m, 3H), 6.81 (d, J=9.4 Hz, 1H), 5.53 (d, J=4.9 Hz, 1H), 5.04 (d, J=6.0 Hz, 1H), 3.09 (m, 1H), 2.81 (m, 1H), 2.33 (m, 1H), 1.73 (dt, J=14.9, 7.5 Hz, 1H).

### Example 44: Synthesis of 5-chloro-N-[2,4-difluoro-3-[1-(4H-1,2,4-triazol-3-yl)imidazo[1,5-a]pyridin-6-yl]phenyl]-2-methoxypyridine-3-sulfonamide (Compound 29)

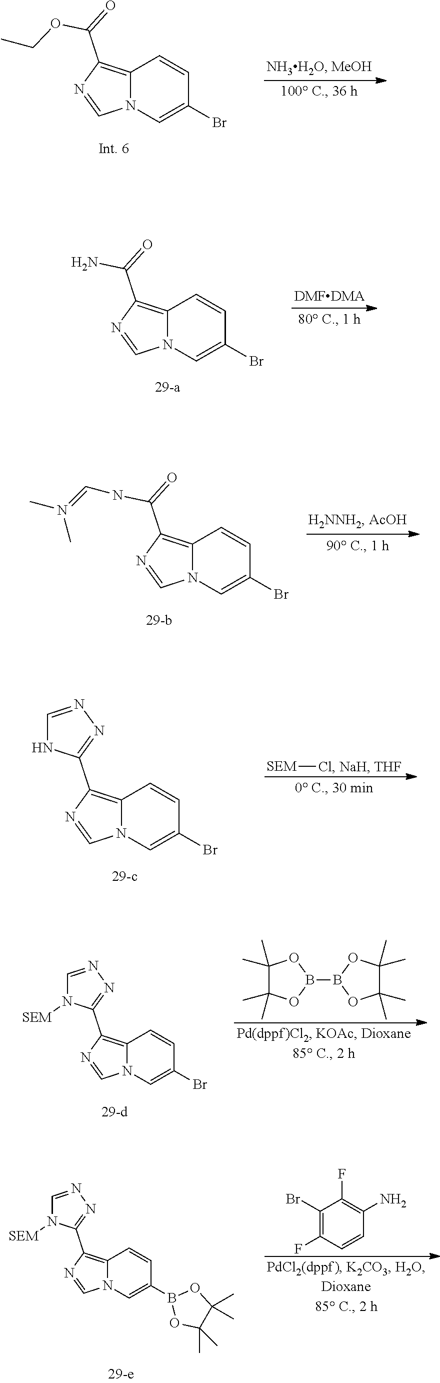

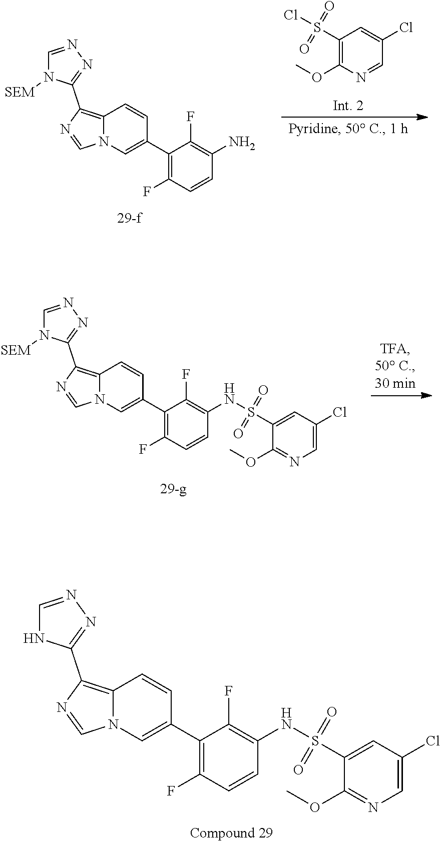 [SMILES: C[Si](C)(C)CCOCn1cnnc1-c1ncn2cc(-c3c(F)ccc(N)c3F)ccc12 ; COc1ncc(Cl)cc1S(=O)(=O)Cl ; COc1ncc(Cl)cc1S(=O)(=O)Nc1ccc(F)c(-c2ccc3c(-c4nncn4COCC[Si](C)(C)C)ncn3c2)c1F ; COc1ncc(Cl)cc1S(=O)(=O)Nc1ccc(F)c(-c2ccc3c(-c4nnc[nH]4)ncn3c2)c1F]

### Synthesis of 29-a: 6-bromoimidazo[1,5-a]pyridine-1-carboxamide

Into a 30 mL sealed tube was placed ethyl 6-bromoimidazo[1,5-a]pyridine-1-carboxylate (500 mg, 1.9 mmol, 1 equiv), MeOH (5 mL) and NH₃·H₂O (5 mL). The resulting solution was stirred for 36 h at 100° C. in an oil bath, then concentrated under vacuum to give 6-bromoimidazo[1,5-a]pyridine-1-carboxamide (360 mg, 61% yield) as a brown solid which was used in next step directly without further purification.

LCMS (ES, m/z): [M+H]⁺: 240

### Synthesis of 29-b: 6-bromo-N-[(1Z)-(dimethylamino)methylidene]imidazo[1,5-a]pyridine-1-carboxamide

Into a 25 mL 3-necked round-bottom flask was placed 6-bromoimidazo[1,5-a]pyridine-1-carboxamide (360 mg, 1.5 mmol, 1 equiv) and DMF-DMA (4 mL). The resulting solution was stirred for 1 h at 80° C. in an oil bath. After cooling, the solids were collected by filtration and dried to give 6-bromo-N-[(1Z)-(dimethylamino)methylidene]imidazo[1,5-a]pyridine-1-carboxamide (167 mg, 33% yield) as a grey solid which was used in next step directly without further purification.

LCMS (ES, m/z): [M+H]⁺: 295

### Synthesis of 29-c: 3-[6-bromoimidazo[1,5-a]pyridin-1-yl]-4H-1,2,4-triazole

Into a 25 mL 3-necked round-bottom flask was placed 6-bromo-N-[(dimethylamino)methylidene]imidazo[1,5-a]pyridine-1-carboxamide (167 mg, 0.56 mmol, 1 equiv), AcOH (2 mL) and NH₂NH₂·H₂O (2 mL). The resulting solution was stirred for 1 h at 90° C. in an oil bath, then concentrated under vacuum. The resulting solution was diluted with H₂O (15 mL). The solution was adjusted to pH 7-8 with saturated NaHCO₃ and was then extracted with dichloromethane (3×15 mL). The combined organics were dried over anhydrous sodium sulfate and concentrated under vacuum to give 3-[6-bromoimidazo[1,5-a]pyridin-1-yl]-4H-1,2,4-triazole (120 mg, crude) as a yellow oil which was used in next step directly without further purification.

LCMS (ES, m/z): [M+H]⁺: 264

### Synthesis of 29-d: 3-[6-bromoimidazo[1,5-a]pyridin-1-yl]-4-[[2-(trimethylsilyl)ethoxy]methyl]-1,2,4-triazole

Into a 25 mL 3-necked round-bottom flask was placed 3-[6-bromoimidazo[1,5-a]pyridin-1-yl]-4H-1,2,4-triazole (120 mg, 0.45 mmol, 1 equiv) in THE (5 mL). This was followed by the addition of 60% NaH (37 mg, 0.9 mmol, 2 eq), in portions at 0° C. To this was added SEM-Cl (114 mg, 0.68 mmol, 1.5 equiv) dropwise with stirring at 0° C. The resulting solution was stirred for 1 h at 0° C. in an ice/salt bath. The reaction was then quenched by the addition of water (20 mL) and the resulting solution was extracted with dichloromethane (3×20 mL). The combined organics were washed with brine (50 ml), dried over anhydrous sodium sulfate and concentrated under vacuum. The residue was applied to a silica gel column, eluting with THF:PE (1:1) to give 3-[6-bromoimidazo[1,5-a]pyridin-1-yl]-4-[[2-(trimethylsilyl)ethoxy]methyl]-1,2,4-triazole (125 mg, 59% yield) as a yellow solid.

LCMS (ES, m/z): [M+H]⁺: 394

### Synthesis of 29-e: 3-[6-(4,4,5,5-tetramethyl-1,3,2-dioxaborolan-2-yl)imidazo[1,5-a]pyridin-1-yl]-4-[[2-(trimethylsilyl)ethoxy]methyl]-1,2,4-triazole

Into a 25 mL 3-necked round-bottom flask, purged and maintained with an inert atmosphere of nitrogen, was placed 3-[6-bromoimidazo[1,5-a]pyridin-1-yl]-4-[[2-(trimethylsilyl)ethoxy]methyl]-1,2,4-triazole (125 mg, 0.32 mmol, 1 equiv), bis(pinacolato)diboron (121 mg, 0.5 mmol, 1.5 equiv), Pd(dppf)Cl₂ (23 mg, 0.03 mmol, 0.1 equiv), KOAc (93 mg, 0.95 mmol, 3 equiv) and dioxane (5 mL). The resulting solution was stirred for 2 h at 85° C. in an oil bath, then cooled and diluted with DCM (20 mL). The mixture was washed with H₂O (2×20 ml) and brine (20 mL), then dried over anhydrous sodium sulfate and concentrated under vacuum. 3-[6-(4,4,5,5-Tetramethyl-1,3,2-dioxaborolan-2-yl)imidazo[1,5-a]pyridin-1-yl]-4-[[2-(trimethylsilyl)ethoxy]methyl]-1,2,4-triazole (200 mg, crude) was isolated as a brown oil which was used in next step directly without further purification.

LCMS (ES, m/z): [M+H]⁺: 442

### Synthesis of 29-f: 2,4-difluoro-3-[1-(4-[[2-(trimethylsilyl)ethoxy]methyl]-1,2,4-triazol-3-yl)imidazo[1,5-a]pyridin-6-yl]aniline

Into a 25 mL 3-necked round-bottom flask, purged and maintained with an inert atmosphere of nitrogen, was placed 3-[6-(4,4,5,5-tetramethyl-1,3,2-dioxaborolan-2-yl)imidazo[1,5-a]pyridin-1-yl]-4-[[2-(trimethylsilyl)ethoxy]methyl]-1,2,4-triazole (200 mg, 0.45 mmol, 1 equiv), 3-bromo-2,4-difluoroaniline (94 mg, 0.45 mmol, 1 equiv), Pd(dppf)Cl₂ (33 mg, 0.045 mmol, 0.1 equiv), K₂CO₃ (188 mg, 1.4 mmol, 3 equiv), H₂O (2 mL) and dioxane (10 mL). The resulting solution was stirred for 2 h at 85° C. in an oil bath, then concentrated under vacuum. The residue was applied to a silica gel column, eluting with THF:PE (35:65). 2,4-Difluoro-3-[1-(4-[[2-(trimethylsilyl)ethoxy]methyl]-1,2,4-triazol-3-yl)imidazo[1,5-a]pyridin-6-yl]aniline (100 mg, 50% yield) was obtained as yellow oil.

LCMS (ES, m/z): [M+H]⁺: 443

### Synthesis of 29-g: 5-chloro-N-[2,4-difluoro-3-[1-(4-[[2-(trimethylsilyl)ethoxy]methyl]-1,2,4-triazol-3-yl)imidazo[1,5-a]pyridin-6-yl]phenyl]-2-methoxypyridine-3-sulfonamide

Into a 25 mL 3-necked round-bottom flask was placed 2,4-difluoro-3-[1-(4-[[2-(trimethylsilyl)ethoxy]methyl]-1,2,4-triazol-3-yl)imidazo[1,5-a]pyridin-6-yl]aniline (100 mg, 0.23 mmol, 1 equiv), 5-chloro-2-methoxypyridine-3-sulfonyl chloride (55 mg, 0.23 mmol, 1 equiv) and pyridine (3 mL). The resulting solution was stirred for 1 h at 50° C. in an oil bath, then cooled and concentrated under vacuum. The residue was applied to a silica gel column, eluting with THF:PE (3:7). 5-Chloro-N-[2,4-difluoro-3-[1-(4-[[2-(trimethylsilyl)ethoxy]methyl]-1,2,4-triazol-3-yl)imidazo[1,5-a]pyridin-6-yl]phenyl]-2-methoxypyridine-3-sulfonamide (87 mg, 80% yield) was obtained as a yellow oil.

LCMS (ES, m/z): [M+H]⁺: 648

### Synthesis of Compound 29: 5-chloro-N-[2,4-difluoro-3-[1-(4H-1,2,4-triazol-3-yl)imidazo[1,5-a]pyridin-6-yl]phenyl]-2-methoxypyridine-3-sulfonamide

Into a 25 mL 3-necked round-bottom flask was placed 5-chloro-N-[2,4-difluoro-3-[1-(4-[[2-(trimethylsilyl)ethoxy]methyl]-1,2,4-triazol-3-yl)imidazo[1,5-a]pyridin-6-yl]phenyl]-2-methoxypyridine-3-sulfonamide (87 mg, 0.14 mmol, 1 equiv) and TFA (2 mL). The resulting solution was stirred for 30 min at 50° C. in an oil bath, then was cooled and concentrated under vacuum. The resulting solution was diluted with EA (15 mL), and the pH adjusted to 7-8 with saturated NaHCO₃/H₂O. The resulting solution was extracted with ethyl acetate (10 mL) and the combined organics dried over anhydrous sodium sulfate, then concentrated under vacuum. The crude product was purified by Flash-Prep-HPLC with the following conditions: column, welch Vltimate XB-C18, 50×250 mm, 10 μm; mobile phase: 35-70% MeCN/0.1% aqueous formic acid; Detector, 220 nm. 5-Chloro-N-[2,4-difluoro-3-[1-(4H-1,2,4-triazol-3-yl)imidazo[1,5-a]pyridin-6-yl]phenyl]-2-methoxypyridine-3-sulfonamide (14.2 mg, 19% yield) was isolated as an off-white solid.

LCMS (ES, m/z): [M+H]⁺: 518

¹H NMR (300 MHz, DMSO-d₆) δ 14.33 (s, 1H), 10.49 (s, 1H), 8.56 (d, J=35.8 Hz, 3H), 8.22 (d, J=9.5 Hz, 1H), 8.09 (s, 1H), 8.02 (s, 1H), 7.37 (q, J=7.7 Hz, 1H), 7.26 (d, J=9.2 Hz, 1H), 7.01 (t, J=5.7 Hz, 1H), 3.92 (s, 3H).

### Example 45: Synthesis of 7-[3-(5-chloro-2-methoxypyridine-3-sulfonamido)-2,6-difluorophenyl]-N-methylimidazo[1,5-a]pyridine-3-carboxamide (Compound 30)

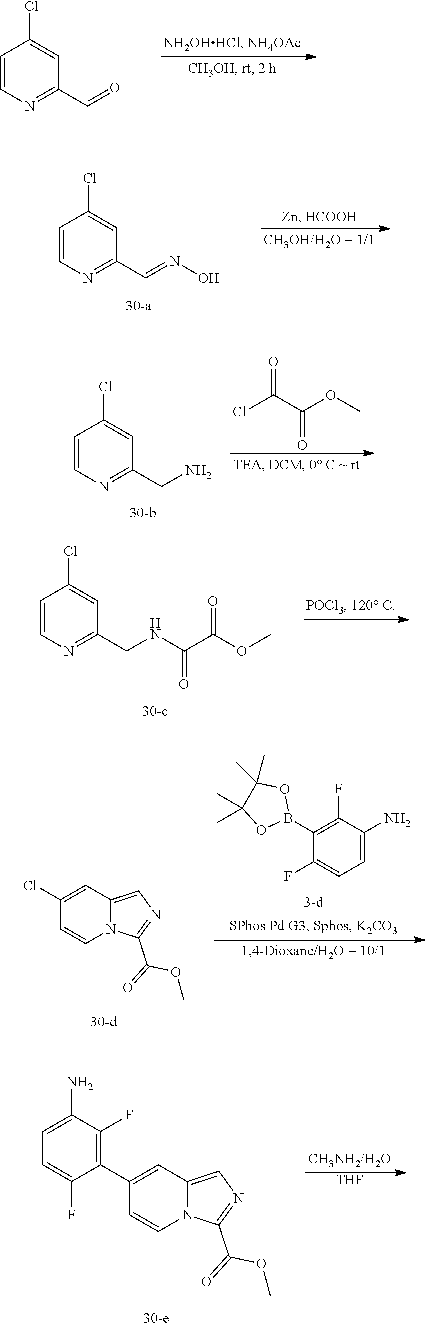

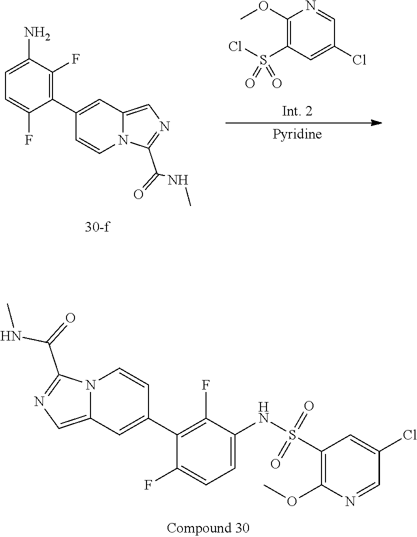 [SMILES: CNC(=O)c1ncc2cc(-c3c(F)ccc(N)c3F)ccn12 ; COc1ncc(Cl)cc1S(=O)(=O)Cl ; CNC(=O)c1ncc2cc(-c3c(F)ccc(NS(=O)(=O)c4cc(Cl)cnc4OC)c3F)ccn12]

### Synthesis of 30-a: N-[(4-chloropyridin-2-yl)methylidene]hydroxylamine

A solution of 4-chloropyridine-2-carbaldehyde (5 g, 35 mmol, 1 equiv), NH₂OH HCl (3.7 g, 53 mmol, 1.5 equiv) and NH₄OAc (8.2 g, 106 mmol, 3 equiv) in CH₃OH (50 mL) was stirred for 2 h at room temperature. The resulting mixture was concentrated under reduced pressure. The residue was suspended in H₂O (50 mL), and the mixture basified to pH 8 with saturated aqueous NaHCO₃. The resulting mixture was extracted with EA (3×50 mL). The combined organics were washed with brine (100 mL), dried over anhydrous Na₂SO₄ and concentrated under reduced pressure to afford N-[(4-chloropyridin-2-yl)methylidene]hydroxylamine (5.3 g, 94% yield) as a white solid which was used in the next step directly without further purification.

LCMS (ES, m/z): [M+H]⁺: 157

### Synthesis of 30-b: 1-(4-chloropyridin-2-yl)methanamine

To a stirred solution of N-[(4-chloropyridin-2-yl)methylidene]hydroxylamine (5.3 g, 34 mmol, 1 equiv) and HCOOH (7.7 g, 168 mmol, 5 equiv) in CH₃OH (25 mL) and H₂O (25 mL) were added Zn powder (11 g, 168 mmol, 5 equiv) in portions at 0° C. The resulting mixture was stirred for 1 h at 0° C., then filtered. The filtrate was concentrated under reduced pressure to afford 1-(4-chloropyridin-2-yl)methanamine (5.7 g, crude) as a brownish yellow oil which was used in the next step directly without further purification.

LCMS (ES, m/z): [M+H]⁺: 143

### Synthesis of 30-c: methyl[[(4-chloropyridin-2-yl)methyl]carbamoyl]formate

To a stirred solution of 1-(4-chloropyridin-2-yl)methanamine (5.2 g, 36 mmol, 1 equiv) and TEA (18.5 g, 182 mmol, 5 equiv) in DCM (50 mL) was added methyl oxalochloridate (4.5 g, 36 mmol, 1 equiv) dropwise at 0° C. The resulting mixture was stirred for 0.5 h at room temperature, then concentrated under reduced pressure. The residue was purified by silica gel column chromatography, eluting with PE:EA (10:1) to afford methyl[[(4-chloropyridin-2-yl)methyl]carbamoyl]formate (2.9 g, 35% yield) as a white solid.

LCMS (ES, m/z): [M+H]⁺: 229

### Synthesis of 30-d: methyl 7-chloroimidazo[1,5-a]pyridine-3-carboxylate

A solution of methyl[[(4-chloropyridin-2-yl)methyl]carbamoyl]formate (2.9 g, 13 mmol, 1 equiv) in POCl₃ (10 mL) was stirred overnight at 120° C. The mixture was allowed to cool to room temperature and was concentrated under reduced pressure. The residue was quenched by the addition of H₂O (50 mL) at 5° C. The resulting mixture was extracted with EA (3×20 mL), and the combined organics were washed with brine (1×30 mL), dried over anhydrous Na₂SO₄, then concentrated under reduced pressure. The residue was purified by silica gel column chromatography, eluting with PE:EA (1:1) to afford methyl 7-chloroimidazo[1,5-a]pyridine-3-carboxylate (0.7 g, 26% yield) as a yellow solid.

LCMS (ES, m/z): [M+H]⁺: 211

### Synthesis of 30-e: methyl 7-(3-amino-2,6-difluorophenyl)imidazo[1,5-a]pyridine-3-carboxylate

To a stirred solution of methyl 7-chloroimidazo[1,5-a]pyridine-3-carboxylate (190 mg, 0.9 mmol, 1 equiv) and 2,4-difluoro-3-(4,4,5,5-tetramethyl-1,3,2-dioxaborolan-2-yl)aniline (460 mg, 1.8 mmol, 2 equiv) in dioxane (2 mL) and H₂O (0.2 mL) were added K₂CO₃ (187 mg, 1.4 mmol, 1.5 equiv), Sphos (74 mg, 0.18 mmol, 0.2 equiv) and SPhos Pd G3 (70 mg, 0.09 mmol, 0.1 equiv) in portions at room temperature under nitrogen atmosphere. The resulting mixture was stirred for 3 h at 90° C., then cooled and quenched with water (10 mL). The resulting mixture was extracted with EA (3×20 mL), and the combined organics washed with brine (10 mL), then dried over anhydrous Na₂SO₄. Concentration afforded a residue which was purified by silica gel column chromatography, eluting with PE: EA (3:1) to afford methyl 7-(3-amino-2,6-difluorophenyl)imidazo[1,5-a]pyridine-3-carboxylate (210 mg, 77% yield) as a light yellow solid.

LCMS (ES, m/z): [M+H]⁺: 304

### Synthesis of 30-f: 7-(3-amino-2,6-difluorophenyl)-N-methylimidazo[1,5-a]pyridine-3-carboxamide

A solution of methyl 7-(3-amino-2,6-difluorophenyl)imidazo[1,5-a]pyridine-3-carboxylate (100 mg, 0.33 mmol, 1 equiv) in tetrahydrofuran (1.0 mL) and 30% aqueous CH₃NH₂ (1.0 mL) was stirred for 0.5 h at room temperature. The reaction mixture was concentrated under reduced pressure to afford 7-(3-amino-2,6-difluorophenyl)-N-methylimidazo[1,5-a]pyridine-3-carboxamide (100 mg, crude) as a yellow oil which was used in the next step directly without further purification.

LCMS (ES, m/z): [M+H]⁺: 303

### Synthesis of Compound 30: 7-[3-(5-chloro-2-methoxypyridine-3-sulfonamido)-2,6-difluorophenyl]-N-methylimidazo[1,5-a]pyridine-3-carboxamide

To a stirred solution of 7-(3-amino-2,6-difluorophenyl)-N-methylimidazo[1,5-a]pyridine-3-carboxamide (100 mg, 0.33 mmol, 1 equiv) in pyridine (2 mL) was added a solution of 5-chloro-2-methoxypyridine-3-sulfonyl chloride (96 mg, 0.4 mmol, 1.2 equiv) in DCM (1 mL) dropwise at 0° C. The resulting mixture was stirred for 2 h at room temperature, then concentrated under vacuum. The residue was purified by prep-HPLC with the following conditions: Column, welch Vltimate XB-C18, 50×250 mm, 10 μm; Mobile Phase: 20-65% MeCN/0.1% aqueous formic acid; detector, 220 nm; to afford 7-[3-(5-chloro-2-methoxypyridine-3-sulfonamido)-2,6-difluorophenyl]-N-methylimidazo[1,5-a]pyridine-3-carboxamide (100 mg, 60% yield) as a white solid.

LCMS (ES, m/z): [M+H]⁺: 508

¹H NMR (300 MHz, DMSO-d₆) δ 10.47 (s, 1H), 9.43 (dd, J=7.5, 1.1 Hz, 1H), 8.60 (d, J=4.8 Hz, 1H), 8.49 (d, J=2.6 Hz, 1H), 8.08 (d, J=2.7 Hz, 1H), 7.83 (s, 1H), 7.69 (d, J=0.9 Hz, 1H), 7.35 (td, J=9.1, 6.0 Hz, 1H), 7.21 (t, J=9.4 Hz, 1H), 6.91 (dd, J=7.5, 1.6 Hz, 1H), 3.91 (s, 3H), 2.84 (d, J=4.8 Hz, 3H).

### Example 46: Synthesis of 5-chloro-N-[2,4-difluoro-3-[1-(1H-imidazol-2-yl)imidazo[1,5-a]pyrazin-6-yl]phenyl]-2-methoxypyridine-3-sulfonamide (Compound 31)

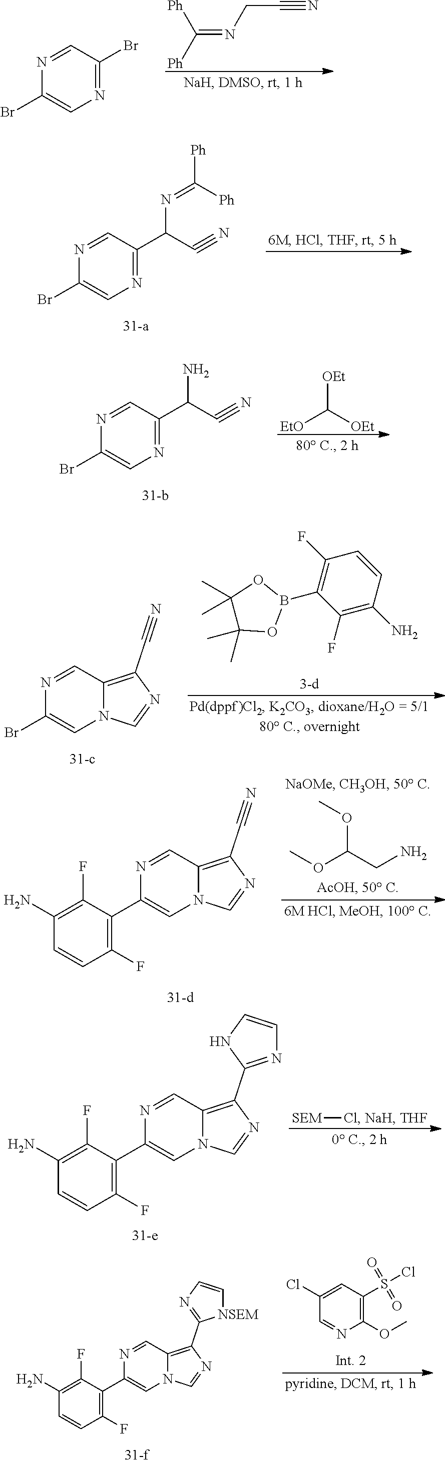

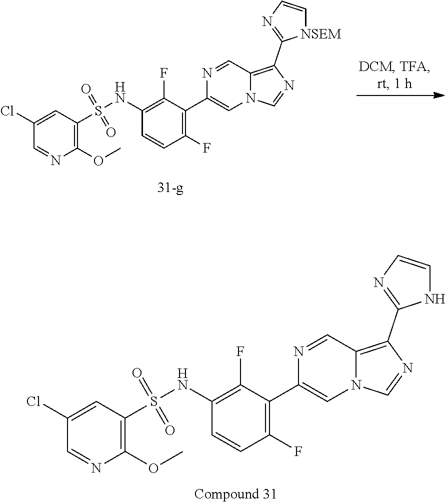 [SMILES: COc1ncc(Cl)cc1S(=O)(=O)Nc1ccc(F)c(-c2cn3cnc(-c4nccn4COCC[Si](C)(C)C)c3cn2)c1F ; COc1ncc(Cl)cc1S(=O)(=O)Nc1ccc(F)c(-c2cn3cnc(-c4ncc[nH]4)c3cn2)c1F]

### Synthesis of 31-a: 2-(5-bromopyrazin-2-yl)-2-[(diphenylmethylidene)amino]acetonitrile

Into a 1000 mL 3-necked round-bottom flask was placed DMSO (400 mL) and NaH (6.7 g, 168 mmol, 2 equiv, 60% in oil). This was followed by the addition of a solution of 2-[(diphenylmethylidene)amino]acetonitrile (22 g, 100 mmol, 1.2 equiv) in DMSO (20 mL) dropwise with stirring at 0° C. After 20 mins, a solution of 2,5-dibromopyrazine (20 g, 84 mmol, 1 equiv) in DMSO (20 mL) was added dropwise at 0° C. The resulting solution was stirred for 2 h at room temperature, then quenched by the addition of 400 mL of saturated NH₄Cl. The resulting solution was extracted with 2×400 mL of ethyl acetate and the combined organics washed with 3×400 ml of water. The organics were dried over anhydrous sodium sulfate and concentrated. The residue was applied to a silica gel column eluting with ethyl acetate/petroleum ether (1/10) to give 2-(5-bromopyrazin-2-yl)-2-[(diphenylmethylidene)amino]acetonitrile (32 g, 81% yield) as a brown oil.

LCMS (ES, m/z): [M+H]⁺: 377

### Synthesis of 31-b: 2-amino-2-(5-bromopyrazin-2-yl)acetonitrile

Into a 250 mL round-bottom flask was placed 2-(5-bromopyrazin-2-yl)-2-[(diphenylmethylidene)amino]acetonitrile (13 g, 34 mmol, 1 equiv), THE (20 mL) and 6 M aqueous HCl (100 mL). The resulting solution was stirred for 5 h at 25° C., then extracted with 2×100 mL of dichloromethane. The pH of the aqueous was adjusted to 8 with NH₃·H₂O and this was extracted with 3×100 mL of dichloromethane. The combined organics were dried over anhydrous sodium sulfate and concentrated to give 2-amino-2-(5-bromopyrazin-2-yl)acetonitrile (5.3 g, 72% yield) as a brown solid which was used in next step directly without further purification.

LCMS (ES, m/z): [M+H]⁺: 213

### Synthesis of 31-c: 6-bromoimidazo[1,5-a]pyrazine-1-carbonitrile

Into a 50 mL round-bottom flask was placed 2-amino-2-(5-bromopyrazin-2-yl)acetonitrile (4.2 g, 20 mmol, 1 equiv) and triethyl orthoformate (10 mL). The resulting solution was stirred for 2 h at 100° C. in an oil bath then cooled and filtered. The solids were dried to give 6-bromoimidazo[1,5-a]pyrazine-1-carbonitrile (1.2 g, 27% yield) as a yellow/brown solid which was used in next step directly without further purification.

LCMS (ES, m/z): [M+H]⁺: 223

### Synthesis of 31-d: 6-(3-amino-2,6-difluorophenyl)imidazo[1,5-a]pyrazine-1-carbonitrile

Into a 50 mL round-bottom flask, purged and maintained with an inert atmosphere of nitrogen, was placed 6-bromoimidazo[1,5-a]pyrazine-1-carbonitrile (930 mg, 4 mmol, 1 equiv), 2,4-difluoro-3-(4,4,5,5-tetramethyl-1,3,2-dioxaborolan-2-yl)aniline (2.1 g, 8 mmol, 2 equiv), Sphos (342 mg, 0.8 mmol, 0.2 equiv), Sphos Pd Gen.3 (325 mg, 0.4 mmol, 0.1 equiv), dioxane (20 mL), H₂O (4 mL) and K₂CO₃ (1.7 g, 12 mmol, 3 equiv). The resulting solution was stirred for 2 h at 80° C. in an oil bath, then cooled and filtered. The filtrate was diluted with water (10 ml) and extracted with DCM (3×10 ml). The combined organics were dried over anhydrous sodium sulfate and concentrated. 6-(3-Amino-2,6-difluorophenyl)imidazo[1,5-a]pyrazine-1-carbonitrile (800 mg, 70% yield) was obtained as a brown solid which was used in next step directly without further purification.

LCMS (ES, m/z): [M+H]⁺: 272

### Synthesis of 31-e: 2,4-difluoro-3-[1-(1H-imidazol-2-yl)imidazo[1,5-a]pyrazin-6-yl]aniline

Into a 50 mL round-bottom flask was placed 6-(3-amino-2,6-difluorophenyl)imidazo[1,5-a]pyrazine-1-carbonitrile (680 mg, 2.5 mmol, 1 equiv), MeOH (25 mL) and MeONa (451 mg, 2.5 mmol, 1 equiv, 30% in MeOH). The resulting solution was stirred for 3 h at 50° C., then cooled to room temperature. 2,2-Dimethoxyethanamine (395 mg, 3.7 mmol, 1.5 equiv) and AcOH (301 mg, 2 mmol, 2 equiv) were added sequentially, and the resulting solution stirred for 1 h at 50° C. After cooling to room temperature, HCl (6 M) (2 mL) and MeOH (5 mL) were added. The resulting solution was stirred for 5 h at 100° C., then concentrated. The residue was suspended in water (20 ml), and the pH adjusted to 8 with 30% aqueous NaOH. The solid formed was collected by filtration and dried to give 2,4-difluoro-3-[1-(1H-imidazol-2-yl)imidazo[1,5-a]pyrazin-6-yl]aniline (660 mg, 84% yield) as a yellow solid which was used in next step directly without further purification.

LCMS (ES, m/z): [M+H]⁺: 313

### Synthesis of 31-f: 2,4-difluoro-3-[1-(1-[[2-(trimethylsilyl)ethoxy]methyl]imidazol-2-yl)imidazo[1,5-a]pyrazin-6-yl]aniline

Into a 50 mL 3-necked round-bottom flask was placed 2,4-difluoro-3-[1-(1H-imidazol-2-yl)imidazo[1,5-a]pyrazin-6-yl]aniline (660 mg, 2 mmol, 1 equiv) and DMF (20 mL). NaH (127 mg, 3 mmol, 1.5 equiv, 60% in oil) was added in portions at 0° C. To this was added SEM-Cl (528 mg, 3 mmol, 1.5 equiv) dropwise with stirring at 0° C. and the mixture was stirred in an ice bath for 3 h. The reaction was quenched by the addition of 30 mL of water, and extracted with 3×30 mL of ethyl acetate. The combined organics were washed with water (30 ml) and dried over anhydrous sodium sulfate, before being concentrated. The residue was applied to a silica gel column, eluting with ethyl acetate/petroleum ether (1/2), to give 2,4-difluoro-3-[1-(1-[[2-(trimethylsilyl)ethoxy]methyl]imidazol-2-yl)imidazo[1,5-a]pyrazin-6-yl]aniline (400 mg, 43% yield) as a yellow solid.

LCMS (ES, m/z): [M+H]⁺: 443

### Synthesis of 31-g: 5-chloro-N-[2,4-difluoro-3-[1-(1-[[2-(trimethylsilyl)ethoxy]methyl]imidazol-2-yl)imidazo[1,5-a]pyrazin-6-yl]phenyl]-2-methoxypyridine-3-sulfonamide

Into an 8 mL vial, was placed 2,4-difluoro-3-[1-(1-[[2-(trimethylsilyl)ethoxy]methyl]imidazol-2-yl)imidazo[1,5-a]pyrazin-6-yl]aniline (200 mg, 0.4 mmol, 1 equiv), DCM (4 mL), pyridine (357 mg, 4.5 mmol, 10 equiv) and 5-chloro-2-methoxypyridine-3-sulfonyl chloride (164 mg, 0.6 mmol, 1.5 equiv). The resulting solution was stirred for 1 h at 25° C., then concentrated. The crude product was purified by Flash-Prep-HPLC with the following conditions: Column, WelFlash™ C18-I, Spherical C18 20-m, 120 g; mobile phase: 5-60% MeCN/0.1% aqueous formic acid; Detector, 220 nm. 5-Chloro-N-[2,4-difluoro-3-[1-(1-[[2-(trimethylsilyl)ethoxy]methyl]imidazol-2-yl)imidazo[1,5-a]pyrazin-6-yl]phenyl]-2-methoxypyridine-3-sulfonamide (130 mg, 44% yield) was isolated as a yellow solid.

LCMS (ES, m/z): [M+H]⁺: 648

### Synthesis of Compound 31: 5-chloro-N-[2,4-difluoro-3-[1-(1H-imidazol-2-yl)imidazo[1,5-a]pyrazin-6-yl]phenyl]-2-methoxypyridine-3-sulfonamide

Into a 50 mL round-bottom flask was placed 5-chloro-N-[2,4-difluoro-3-[1-(1-[[2-(trimethylsilyl)ethoxy]methyl]imidazol-2-yl)imidazo[1,5-a]pyrazin-6-yl]phenyl]-2-methoxypyridine-3-sulfonamide (160 mg, 0.2 mmol, 1 equiv), DCM (6 mL) and TFA (2 mL). The resulting solution was stirred for 1 h at 25° C., then concentrated. The residue was purified by HPLC using the following conditions: Column, welch Vltimate XB-C18, 50×250 mm, 10 μm; Mobile Phase 35-70% MeCN/0.1% aqueous formic acid; Detector, 220 nm. 5-Chloro-N-[2,4-difluoro-3-[1-(1H-imidazol-2-yl)imidazo[1,5-a]pyrazin-6-yl]phenyl]-2-methoxypyridine-3-sulfonamide (70 mg 62% yield) was isolated as a yellow solid.

LCMS (ES, m/z): [M+H]⁺: 518

¹H NMR (300 MHz, DMSO-d₆) δ 12.72 (s, 1H), 10.44 (s, 1H), 9.55 (d, J=1.6 Hz, 1H), 8.66 (s, 1H), 8.56 (s, 1H), 8.49 (d, J=2.6 Hz, 1H), 8.09 (d, J=2.6 Hz, 1H), 7.40 (td, J=8.8, 5.8 Hz, 1H), 7.22 (t, J=9.0 Hz, 1H), 7.18 (s, 2H), 3.92 (s, 3H).

### Example 47: Synthesis of 5-chloro-N-[2,4-difluoro-3-[1-(2H-pyrazol-3-yl)imidazo[1,5-a]pyridin-6-yl]phenyl]-2-methoxypyridine-3-sulfonamide (Compound 32)

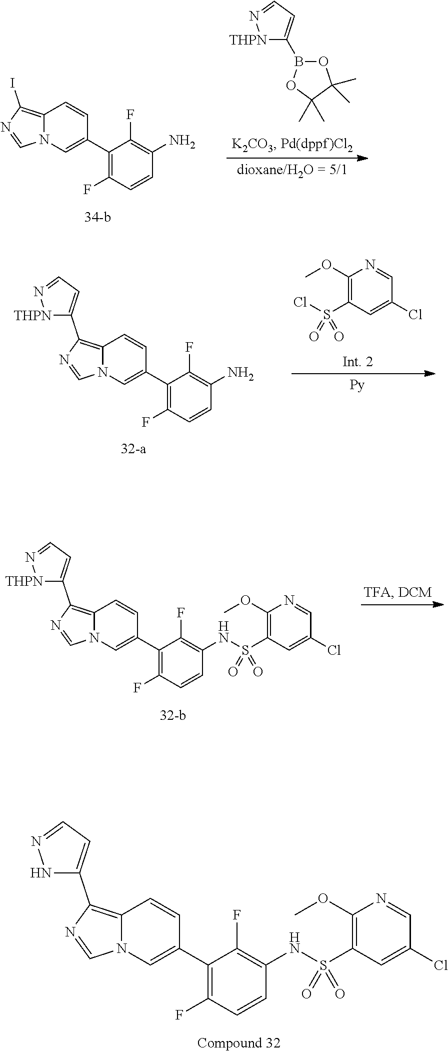

### Synthesis of 32-a: 2,4-difluoro-3-[1-[2-(oxan-2-yl)pyrazol-3-yl]imidazo[1,5-a]pyridin-6-yl]aniline

To a stirred solution of 2,4-difluoro-3-[1-iodoimidazo[1,5-a]pyridin-6-yl]aniline (100 mg, 0.27 mmol, 1 equiv) and 1-(oxan-2-yl)-5-(4,4,5,5-tetramethyl-1,3,2-dioxaborolan-2-yl)pyrazole (150 mg, 0.539 mmol, 2 equiv) in dioxane (5 mL) and H₂O (1 mL) were added Pd(dppf)Cl₂ (20 mg, 0.027 mmol, 0.1 equiv) and K₂CO₃ (75 mg, 0.539 mmol, 2 equiv) under N₂ atmosphere. The reaction was then stirred at 80° C. for 5 h. The reaction was cooled to room temperature and H₂O (3 mL) was added. The mixture was extracted with EA (3×5 mL), and the combined organic layers were washed with brine (2×5 mL), dried over anhydrous Na₂SO₄ and concentrated under reduced pressure. The residue was purified by silica gel column chromatography, eluting with PE:EA (1:1) to afford 2,4-difluoro-3-[1-[2-(oxan-2-yl)pyrazol-3-yl]imidazo[1,5-a]pyridin-6-yl]aniline (90 mg, 84% yield) as a yellow solid.

LCMS (ES, m/z): [M+H]⁺: 396

### Synthesis of 32-b: 5-chloro-N-(2,4-difluoro-3-[1-[2-(oxan-2-yl)pyrazol-3-yl]imidazo[1,5-a]pyridin-6-yl]phenyl)-2-methoxypyridine-3-sulfonamide

A solution of 2,4-difluoro-3-[1-[2-(oxan-2-yl)pyrazol-3-yl]imidazo[1,5-a]pyridin-6-yl]aniline (90 mg, 0.228 mmol, 1 equiv) and 5-chloro-2-methoxypyridine-3-sulfonyl chloride (166 mg, 0.683 mmol, 3 equiv) in pyridine (4 mL) was stirred for 2 h at room temperature.

The resulting mixture was concentrated under reduced pressure to give 5-chloro-N-(2,4-difluoro-3-[1-[2-(oxan-2-yl)pyrazol-3-yl]imidazo[1,5-a]pyridin-6-yl]phenyl)-2-methoxypyridine-3-sulfonamide (60 mg, crude) as a yellow oil which was used in the next step directly without further purification.

LCMS (ES, m/z): [M+H]⁺: 601

### Synthesis of Compound 32: 5-chloro-N-[2,4-difluoro-3-[1-(2H-pyrazol-3-yl)imidazo[1,5-a]pyridin-6-yl]phenyl]-2-methoxypyridine-3-sulfonamide

A solution of 5-chloro-N-(2,4-difluoro-3-[1-[2-(oxan-2-yl)pyrazol-3-yl]imidazo[1,5-a]pyridin-6-yl]phenyl)-2-methoxypyridine-3-sulfonamide (60 mg, 0.1 mmol, 1 equiv) in TFA (1 mL) and DCM (3 mL) was stirred for 1 h at room temperature. The reaction mixture was concentrated under reduced pressure. The residue was purified by prep-HPLC with the following conditions: Column, welch Vltimate XB-C18, 50×250 mm, 10 μm; Mobile Phase 30-80% MeCN/0.1% aqueous formic acid; Detector, 220 nm; to afford 5-chloro-N-[2,4-difluoro-3-[1-(2H-pyrazol-3-yl)imidazo[1,5-a]pyridin-6-yl]phenyl]-2-methoxypyridine-3-sulfonamide (18 mg, 35% yield) as a light yellow solid.

LCMS (ES, m/z): [M+H]⁺: 517

¹H NMR (300 MHz, DMSO-d₆) δ 12.81 (s, 1H), 10.47 (s, 1H), 8.53-8.42 (m, 3H), 8.11 (s, 1H), 8.08 (d, J=2.6 Hz, 1H), 7.74 (s, 1H), 7.36 (td, J=8.8, 5.9 Hz, 1H), 7.28-7.16 (m, 1H), 6.78 (d, J=9.4 Hz, 1H), 6.67 (d, J=2.2 Hz, 1H), 3.92 (s, 3H).

### Example 48: Synthesis of (6R)-6-[3-(5-chloro-2-methoxypyridine-3-sulfonamido)-2,6-difluorophenyl]-N,7-dimethyl-5H,6H,8H-imidazo[1,5-a]pyrazine-1-carboxamide and (6S)-6-[3-(5-chloro-2-methoxypyridine-3-sulfonamido)-2,6-difluorophenyl]-N,7-dimethyl-5H,6H,8H-imidazo[1,5-a]pyrazine-1-carboxamide (Compounds 33-1 & 33-2)

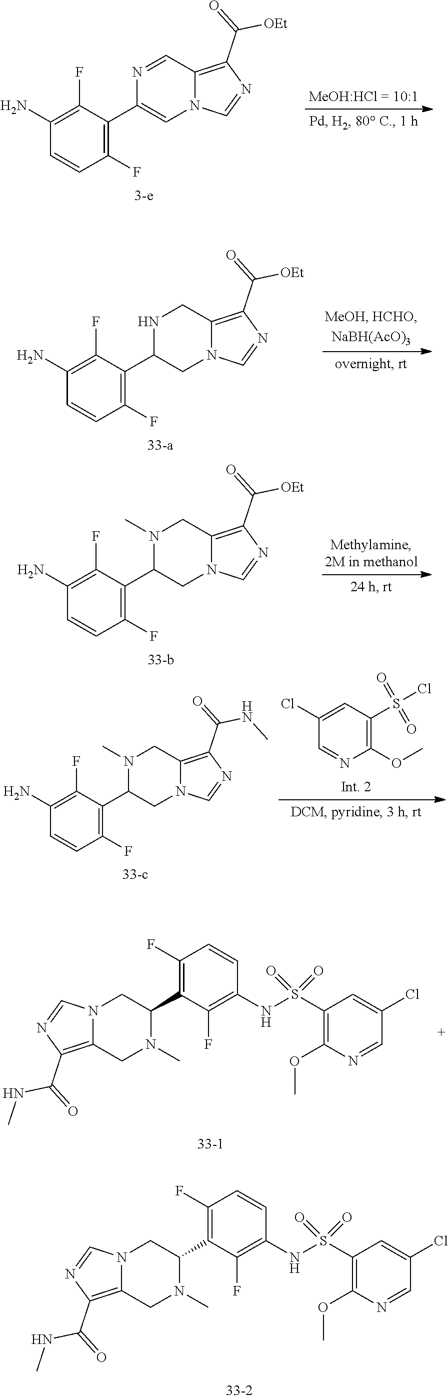

### Synthesis of 33-a: ethyl 6-(3-amino-2,6-difluorophenyl)-5H,6H,7H,8H-imidazo[1,5-a]pyrazine-1-carboxylate

Into a 50 mL pressure tank reactor purged and maintained with an inert atmosphere of nitrogen, was placed ethyl 6-(3-amino-2,6-difluorophenyl)imidazo[1,5-a]pyrazine-1-carboxylate (500 mg), 10% Pd/C (500 mg), MeOH (10 mL) and HCl (1 mL, 12 M). The reactor was charged with H₂ (g) at 20 atm and the reaction stirred for 1 h at 80° C. in an oil bath. The reaction mixture was cooled to room temperature and filtered. The filtrate was concentrated, and the residue purified by prep-HPLC with the following conditions: Column, WelFlash™ C18-I, Spherical C18 20-40 um, 120 g; mobile phase: 20-75% MeCN/0.1% aqueous formic acid; Detector, 220 nm. Ethyl 6-(3-amino-2,6-difluorophenyl)-5H,6H,7H,8H-imidazo[1,5-a]pyrazine-1-carboxylate (400 mg) was isolated as a white solid. LCMS (ES, m/z): [M+H]+: 323

### Synthesis of 33-b: ethyl 6-(3-amino-2,6-difluorophenyl)-7-methyl-5H,6H,8H-imidazo[1,5-a]pyrazine-1-carboxylate

Into a 50 mL 3-necked round-bottom flask was placed ethyl 6-(3-amino-2,6-difluorophenyl)-5H,6H,7H,8H-imidazo[1,5-a]pyrazine-1-carboxylate (400 mg, 1.2 mmol, 1 equiv), MeOH (10 mL) and HCHO (1.07 g, 12.4 mmol, 10 equiv, 37% in water). This was followed by the addition of NaBH(AcO)₃ (526 mg, 2.5 mmol, 2 equiv) in portions at room temperature. The resulting solution was stirred overnight, then filtered. The filtrate was concentrated and the residue purified by Prep-HPLC with the following conditions: Column, WelFlash™ C18-I, Spherical C18 20-40 um, 120 g; mobile phase: 15-75% MeCN/0.1% aqueous formic acid; Detector, 220 nm. Ethyl 6-(3-amino-2,6-difluorophenyl)-7-methyl-5H,6H,8H-imidazo[1,5-a]pyrazine-1-carboxylate (300 mg, 72% yield) was isolated as a white solid.

LCMS (ES, m/z): [M+H]⁺: 337

### Synthesis of 33-c: 6-(3-amino-2,6-difluorophenyl)-N,7-dimethyl-5H,6H,8H-imidazo[1,5-a]pyrazine-1-carboxamide

Into a 50 mL 3-necked round-bottom flask was placed ethyl 6-(3-amino-2,6-difluorophenyl)-7-methyl-5H,6H,8H-imidazo[1,5-a]pyrazine-1-carboxylate (300 mg, 0.9 mmol, 1 equiv). This was followed by the addition of 2 M methylamine in methanol (5 mL) dropwise with stirring at room temperature. The resulting solution was stirred for 24 h at room temperature, then concentrated. The reaction mixture was purified by Prep-HPLC with the following conditions: Column, WelFlash™ C18-I, Spherical C18 20-40 um, 120 g; mobile phase: 25-95% MeCN/0.1% aqueous formic acid; Detector, 220 nm. 6-(3-Amino-2,6-difluorophenyl)-N,7-dimethyl-5H,6H,8H-imidazo[1,5-a]pyrazine-1-carboxamide (200 mg, 70% yield) was obtained as a white solid.

LCMS (ES, m/z): [M+H]⁺: 322

### Synthesis of Compound 33-1 & 33-2: (6R)-6-[3-(5-chloro-2-methoxypyridine-3-sulfonamido)-2,6-difluorophenyl]-N,7-dimethyl-5H,6H,8H-imidazo[1,5-a]pyrazine-1-carboxamide and (6S)-6-[3-(5-chloro-2-methoxypyridine-3-sulfonamido)-2,6-difluorophenyl]-N,7-dimethyl-5H,6H,8H-imidazo[1,5-a]pyrazine-1-carboxamide

Into an 8 mL vial was placed 6-(3-amino-2,6-difluorophenyl)-N,7-dimethyl-5H,6H,8H-imidazo[1,5-a]pyrazine-1-carboxamide (150 mg, 0.5 mmol, 1 equiv), DCM (3.00 mL), pyridine (369 mg, 4.7 mmol, 10 equiv) and 5-chloro-2-methoxypyridine-3-sulfonyl chloride (135 mg, 0.5 mmol, 1.2 equiv). The resulting solution was stirred for 3 h at rt, then concentrated. The crude product was purified by Flash-Prep-HPLC with the following conditions: Column, WelFlash™ C18-I, Spherical C18 20-40 um, 120 g; mobile phase: 5-60% MeCN/0.1% aqueous formic acid; Detector, 220 nm. The enantiomers were separated by Chiral-Prep-HPLC with the following conditions: Column, CHIRALPAK IC 250*20 mm; mobile phase, Hexane+0.1% DEA and 50% EtOH; Detector, 254 nm. This gave (6R)-6-[3-(5-chloro-2-methoxypyridine-3-sulfonamido)-2,6-difluorophenyl]-N,7-dimethyl-5H,6H,8H-imidazo[1,5-a]pyrazine-1-carboxamide (15 mg 6% yield) as a white solid and (6S)-6-[3-(5-chloro-2-methoxypyridine-3-sulfonamido)-2,6-difluorophenyl]-N,7-dimethyl-5H,6H,8H-imidazo[1,5-a]pyrazine-1-carboxamide (16 mg, 6.5% yield) as a white solid.

Stereochemistry was randomly assigned.

LCMS (ES, m/z): [M+H]⁺: 527 for both isomers (6R)-6-[3-(5-chloro-2-methoxypyridine-3-sulfonamido)-2,6-difluorophenyl]-N,7-dimethyl-5H,6H,8H-imidazo[1,5-a]pyrazine-1-carboxamide:

¹H NMR (300 MHz, DMSO-d₆) δ 10.36 (s, 1H), 8.50 (d, J=2.6 Hz, 1H), 7.96 (d, J=2.6 Hz, 1H), 7.80 (d, J=4.9 Hz, 1H), 7.59 (s, 1H), 7.42-7.34 (m, 1H), 7.15 (t, J=9.4 Hz, 1H), 4.29 (dd, J=10.9, 3.1 Hz, 1H), 4.20 (d, J=16.3 Hz, 1H), 4.17-4.00 (m, 2H), 3.94 (s, 3H), 3.55 (d, J=16.3 Hz, 1H), 2.72 (d, J=4.7 Hz, 3H), 1.98 (s, 3H).

### (6S)-6-[3-(5-chloro-2-methoxypyridine-3-sulfonamido)-2,6-difluorophenyl]-N,7-dimethyl-5H,6H,8H-imidazo[1,5-a]pyrazine-1-carboxamide

¹H NMR-0B (300 MHz, DMSO-d₆) δ 10.36 (s, 1H), 8.50 (d, J=2.6 Hz, 1H), 7.96 (d, J=2.6 Hz, 1H), 7.80 (d, J=4.9 Hz, 1H), 7.59 (s, 1H), 7.42-7.34 (m, 1H), 7.14 (t, J=9.5 Hz, 1H), 4.29 (dd, J=11.0, 3.2 Hz, 1H), 4.20 (d, J=16.3 Hz, 1H), 4.18-4.00 (m, 2H), 3.94 (s, 3H), 3.55 (d, J=16.4 Hz, 1H), 2.72 (d, J=4.7 Hz, 3H), 1.98 (s, 3H).

### Example 49: Synthesis of 5-chloro-N-[2,4-difluoro-3-[1-(3H-1,2,3-triazol-4-yl)imidazo[1,5-a]pyridin-6-yl]phenyl]-2-methoxypyridine-3-sulfonamide (Compound

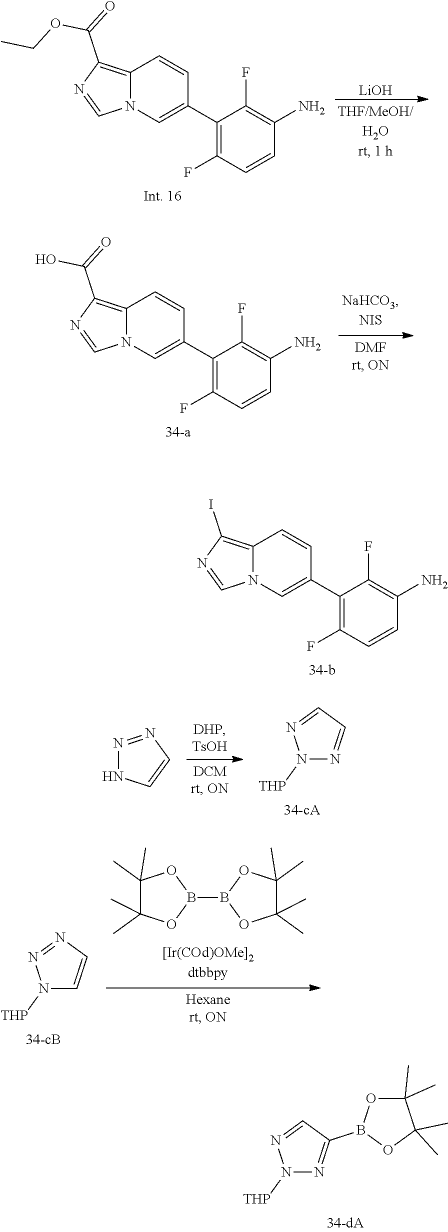

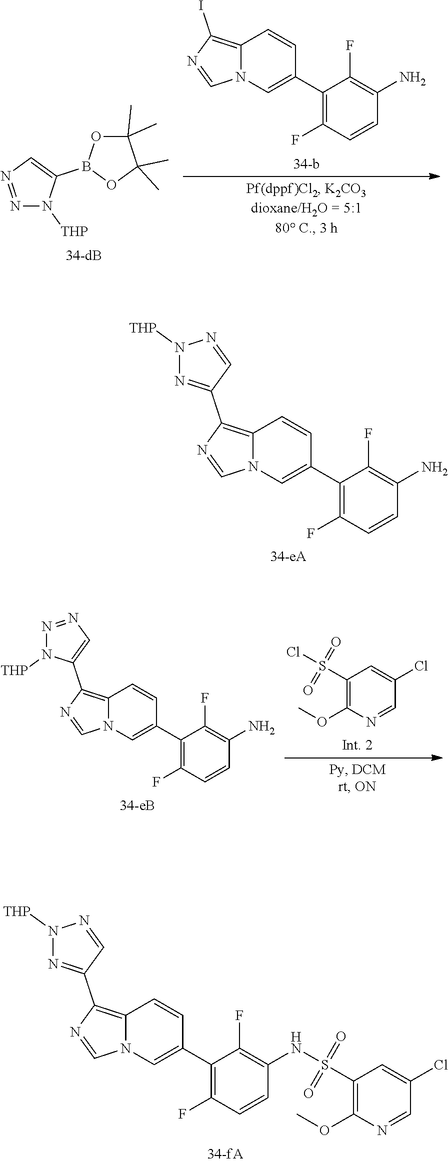

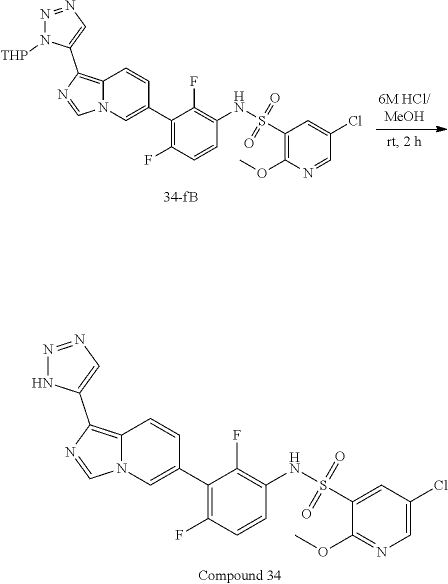

### Synthesis of 34-a: 6-(3-amino-2,6-difluorophenyl)imidazo[1,5-a]pyridine-1-carboxylic acid

To a stirred solution of ethyl 6-(3-amino-2,6-difluorophenyl)imidazo[1,5-a]pyridine-1-carboxylate (2.1 g, 6.6 mmol, 1 equiv) in a mixture solution of THF (8 mL), MeOH (8 mL) and H₂O (4 mL) was added LiOH (0.5 g, 20 mmol, 3 equiv) and the reaction was stirred at room temperature for 1 h. The resulting solution was concentrated under vacuum to remove THE and MeOH, and the residue was diluted with H₂O (10 mL) and then acidified to pH 2 with 2 M HCl (10 mL).

The precipitated solids were collected by filtration and washed with H₂O (10 mL). The filter cake was dried under vacuum to give 6-(3-amino-2,6-difluorophenyl)imidazo[1,5-a]pyridine-1-carboxylic acid (1.8 g, 94% yield) as a brown solid which was used in next step directly without further purification.

LCMS (ES, m/z): [M+H]⁺: 290

### Synthesis of 34-b: 2,4-difluoro-3-[1-iodoimidazo[1,5-a]pyridin-6-yl]aniline

To a stirred solution of 6-(3-amino-2,6-difluorophenyl)imidazo[1,5-a]pyridine-1-carboxylic acid (1.8 g, 6 mmol, 1 equiv) in DMF (20 mL) was added NaHCO₃ (2.1 g, 24 mmol, 4 equiv) and NIS (1.4 g, 6 mmol, 1 equiv) and the reaction stirred at room temperature overnight. The mixture was diluted with H₂O (40 mL) and extracted with EA (50 mL×3). The combined organics were dried over anhydrous Na₂SO₄ and concentrated under reduced pressure, and the residue purified by silica gel column chromatography, eluting with PE:EA (2:1) to give 2,4-difluoro-3-[1-iodoimidazo[1,5-a]pyridin-6-yl]aniline (1.4 g, 60% yield) as a grey solid.

LCMS (ES, m/z): [M+H]⁺: 372

### Synthesis of 34-cA 34-cB: 2-(oxan-2-yl)-1,2,3-triazole and 1-(oxan-2-yl)-1,2,3-triazole

To a stirred solution of 1,2,3-triazole (1 g, 14.5 mmol, 1 equiv) in DCM (48 mL) was added DHP (2.4 g, 29 mmol, 2 equiv) and TsOH (25 mg, 0.145 mmol, 0.01 equiv), and the reaction was stirred at room temperature overnight. H₂O (10 mL) was added the mixture extracted with DCM (30 mL×3). The combined organics were dried over anhydrous Na₂SO₄ and concentrated. The residue was purified by silica gel column chromatography, eluting with PE (5 min) and then EA (10 min) to afford a mixture of 2-(oxan-2-yl)-1,2,3-triazole and 1-(oxan-2-yl)-1,2,3-triazole (2 g, 90% yield) as a yellow oil.

LCMS (ES, m/z): [M+H]⁺: 154

### Synthesis of 34-dA & 34-dB: 2-(oxan-2-yl)-4-(4,4,5,5-tetramethyl-1,3,2-dioxaborolan-2-yl)-1,2,3-triazole and 1-(oxan-2-yl)-5-(4,4,5,5-tetramethyl-1,3,2-dioxaborolan-2-yl)-1,2,3-triazole

To a stirred solution of [Ir(COD)OMe]₂ (26 mg, 0.04 mmol, 0.03 equiv) and dtbbpy (21 mg, 0.08 mmol, 0.06 equiv) in hexane (3.3 mL) was added bis(pinacolato)diboron (365 mg, 1.4 mmol, 1.1 equiv) at room temperature under N₂ atmosphere, and the reaction was stirred at room temperature for 15 min. The mixture of 1-(oxan-2-yl)-1,2,3-triazole) and 2-(oxan-2-yl)-1,2,3-triazole (200 mg, 1.3 mmol, 1 equiv) was added, and the reaction stirred at room temperature overnight. The resulting mixture was filtered and the filter cake was washed with hexane (10 mL). The filtrate was concentrated under reduced pressure to give the mixture of 2-(oxan-2-yl)-4-(4,4,5,5-tetramethyl-1,3,2-dioxaborolan-2-yl)-1,2,3-triazole and 1-(oxan-2-yl)-5-(4,4,5,5-tetramethyl-1,3,2-dioxaborolan-2-yl)-1,2,3-triazole (400 mg, crude) as a red oil.

LCMS (ES, m/z): [M+H]⁺: 280

### Synthesis of 34-eA & 34-eB: 2,4-difluoro-3-[1-[2-(oxan-2-yl)-1,2,3-triazol-4-yl]imidazo[1,5-a]pyridin-6-yl]aniline and 2,4-difluoro-3-[1-[3-(oxan-2-yl)-4,5-dihydro-1,2,3-triazol-4-yl]imidazo[1,5-a]pyridin-6-yl]aniline

To a stirred solution of 2,4-difluoro-3-[1-iodoimidazo[1,5-a]pyridin-6-yl]aniline (170 mg, 0.46 mmol, 1 equiv) in dioxane (1.5 mL) and H₂O (0.3 mL) was added mixture of 1-(oxan-2-yl)-5-(4,4,5,5-tetramethyl-1,3,2-dioxaborolan-2-yl)-1,2,3-triazole) and 2-(oxan-2-yl)-4-(4,4,5,5-tetramethyl-1,3,2-dioxaborolan-2-yl)-1,2,3-triazole (257 mg, 0.92 mmol, 2 equiv). Pd(dppf)Cl₂ (32 mg, 0.046 mmol, 0.1 equiv) and K₂CO₃ (127 mg, 0.92 mmol, 2 equiv) were added, and the resulting solution was degassed with N₂, then stirred at 80° C. for 3 h. The reaction was cooled to room temperature and diluted with H₂O (5 mL), before being extracted with EA (5 mL×3). The combined organics were dried over anhydrous Na₂SO₄ and concentrated under reduced pressure. The residue was purified by silica gel column chromatography, eluting with PE:EA (1:4) to afford a mixture of 2,4-difluoro-3-[1-[2-(oxan-2-yl)-1,2,3-triazol-4-yl]imidazo[1,5-a]pyridin-6-yl]aniline and 2,4-difluoro-3-[1-[3-(oxan-2-yl)-4,5-dihydro-1,2,3-triazol-4-yl]imidazo[1,5-a]pyridin-6-yl]aniline (100 mg, 55% yield) as a yellow solid.

LCMS (ES, m/z): [M+H]⁺: 397

### Synthesis of 34-fA & 34-fB: 5-chloro-N-(2,4-difluoro-3-[1-[2-(oxan-2-yl)-1,2,3-triazol-4-yl]imidazo[1,5-a]pyridin-6-yl]phenyl)-2-methoxypyridine-3-sulfonamide

To a stirred solution of a mixture of 2,4-difluoro-3-[1-[3-(oxan-2-yl)-4,5-dihydro-1,2,3-triazol-4-yl]imidazo[1,5-a]pyridine-6-yl]aniline) and 2,4-difluoro-3-[1-[2-(oxan-2-yl)-1,2,3-triazol-4-yl]imidazo[1,5-a]pyridin-6-yl]aniline (100 mg, 0.25 mmol, 1 equiv) in DCM (5 mL) was added 5-chloro-2-methoxypyridine-3-sulfonyl chloride (61 mg, 0.25 mmol, 1 equiv) and pyridine (60 mg, 0.75 mmol, 3 equiv). The reaction was stirred at room temperature overnight then concentrated. The residue was purified by silica gel column chromatography, eluting with PE:EA (1:1) to afford a mixture of 5-chloro-N-(2,4-difluoro-3-[1-[3-(oxan-2-yl)-1,2,3-triazol-4-yl]imidazo[1,5-a]pyridine-6-yl]phenyl)-2-methoxypyridine-3-sulfonamide and 5-chloro-N-(2,4-difluoro-3-[1-[2-(oxan-2-yl)-1,2,3-triazol-4-yl]imidazo[1,5-a]pyridin-6-yl]phenyl)-2-methoxypyridine-3-sulfonamide (70 mg, 46% yield) as a yellow solid.

LCMS (ES, m/z): [M+H]⁺: 602

### Synthesis of Compound 34: 5-chloro-N-[2,4-difluoro-3-[1-(3H-1,2,3-triazol-4-yl)imidazo[1,5-a]pyridin-6-yl]phenyl]-2-methoxypyridine-3-sulfonamide

A mixture of 5-chloro-N-(2,4-difluoro-3-[1-[3-(oxan-2-yl)-1,2,3-triazol-4-yl]imidazo[1,5-a]pyridin-6-yl]phenyl)-2-methoxypyridine-3-sulfonamide) and 5-chloro-N-(2,4-difluoro-3-[1-[2-(oxan-2-yl)-1,2,3-triazol-4-yl]imidazo[1,5-a]pyridin-6-yl]phenyl)-2-methoxypyridine-3-sulfonamide (70 mg) in 6 M HCl in MeOH (5 mL) was stirred at room temperature for 2 hours. The solution was concentrated directly and the residue was purified by prep-HPLC with the following conditions: Column, welch Vltimate XB-C18, 50×250 mm, 10 μm; Mobile Phase: 25-65% MeCN/0.1% aqueous formic acid; Detector, 220 nm; to give 5-chloro-N-[2,4-difluoro-3-[1-(3H-1,2,3-triazol-4-yl)imidazo[1,5-a]pyridin-6-yl]phenyl]-2-methoxypyridine-3-sulfonamide (27 mg, 45% yield) as a light yellow solid.

LCMS (ES, m/z): [M+H]⁺: 518.

¹H NMR (300 MHz, Methanol-d4) δ 8.47 (s, 1H), 8.38 (s, 1H), 8.37 (d, J=2.6 Hz, 1H), 8.17 (s, 2H), 8.10 (d, J=2.6 Hz, 1H), 7.58-7.50 (m, 1H), 7.13 (td, J=9.2, 1.8 Hz, 1H), 6.90 (d, J=9.8 Hz, 1H), 4.04 (s, 3H).

### Example 50: Synthesis of 5-chloro-N-[2,4-difluoro-3-[3-(1H-imidazol-2-yl)imidazo[1,5-a]pyridin-7-yl]phenyl]-2-methoxypyridine-3-sulfonamide (Compound 35)

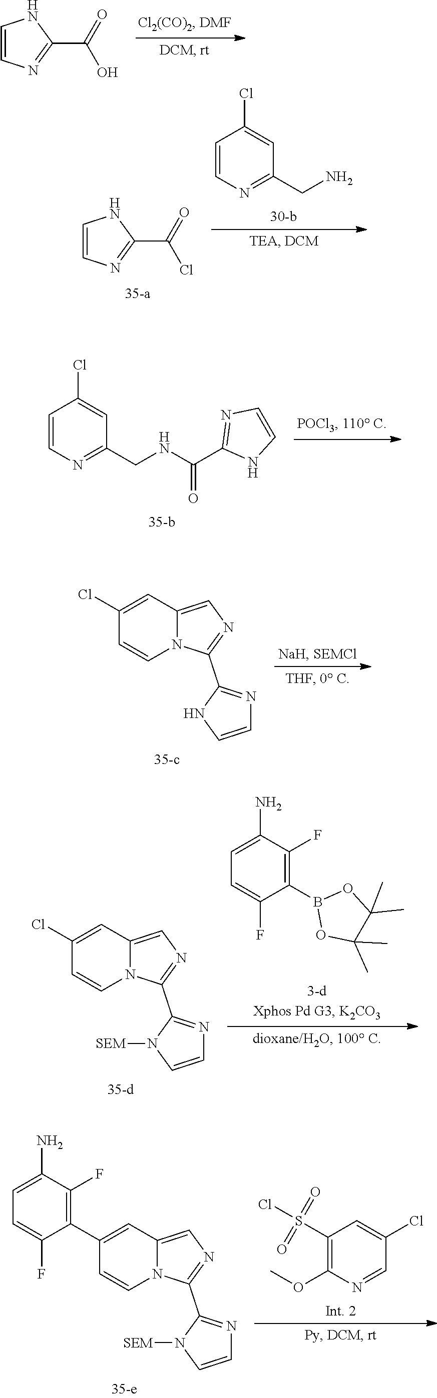

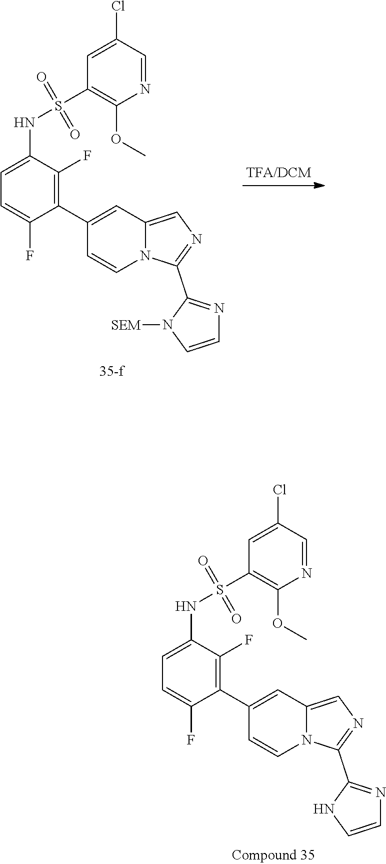 [SMILES: COc1ncc(Cl)cc1S(=O)(=O)Nc1ccc(F)c(-c2ccn3c(-c4nccn4COCC[Si](C)(C)C)ncc3c2)c1F ; COc1ncc(Cl)cc1S(=O)(=O)Nc1ccc(F)c(-c2ccn3c(-c4ncc[nH]4)ncc3c2)c1F]

### Synthesis of 35-a: 1H-imidazole-2-carbonyl chloride

To a stirred solution of 1H-imidazole-2-carboxylic acid (8 g, 71 mmol, 1 equiv) and DMF (2 drops) in DCM (60 mL) were added oxalyl chloride (18 g, 143 mmol, 2 equiv) dropwise at 0° C. The resulting solution was stirred at room temperature for 3 h, then concentrated under reduced pressure to afford 1H-imidazole-2-carbonyl chloride (8 g, crude) as a yellow solid which was used in the next step directly without further purification.

### Synthesis of 35-b: N-[(4-chloropyridin-2-yl)methyl]-1H-imidazole-2-carboxamide

To a stirred solution of 1-(4-chloropyridin-2-yl)methanamine (6 g, 42 mmol, 1 equiv) and TEA (12 g, 126 mmol, 3 equiv) in DCM (100 mL) were added a solution of 1H-imidazole-2-carbonyl chloride (6 g, 50 mmol, 1.2 equiv) in DCM (20 mL) at 0° C. The resulting solution was stirred at room temperature for 1 h. The reaction mixture was concentrated under reduced pressure to give a residue, which was purified by column chromatography over silica gel (eluent: PE:EA=1:1) to afford N-[(4-chloropyridin-2-yl)methyl]-1H-imidazole-2-carboxamide (6 g, 60% yield) as a white solid.

LCMS (ES, m/z): [M+H]⁺: 237

### Synthesis of 35-c: 2-[7-chloroimidazo[1,5-a]pyridin-3-yl]-1H-imidazole

A mixture of N-[(4-chloropyridin-2-yl)methyl]-1H-imidazole-2-carboxamide (4 g, 17 mmol, 1 equiv) in POCl₃ (50 mL) was stirred for 2 h at 110° C. The mixture was allowed to cool to room temperature, then was concentrated and the residue quenched by careful addition of saturated aqueous NaHCO₃ (100 mL). The resulting mixture was extracted with EA (3×200 mL). The combined organics were washed with brine (3×100 mL), dried over anhydrous Na₂SO₄ and concentrated under reduced pressure. The residue was purified by silica gel column chromatography, eluting with PE:EA (1:5) to afford 2-[7-chloroimidazo[1,5-a]pyridin-3-yl]-1H-imidazole (1.7 g, 46% yield) as a brown solid.

LCMS (ES, m/z): [M+H]⁺: 219

### Synthesis of 35-d: 2-[7-chloroimidazo[1,5-a]pyridin-3-yl]-1-[[2-(trimethylsilyl)ethoxy]methyl]imidazole

To a stirred solution of 2-[7-chloroimidazo[1,5-a]pyridin-3-yl]-1H-imidazole (1.7 g, 7.7 mmol, 1 equiv) in THE (50 mL) was added NaH (621 mg, 15.5 mmol, 2 equiv, 60% in oil) portion wise at 0° C. under nitrogen atmosphere. The reaction mixture was stirred for 0.5 h at 0° C., then SEM-Cl (1.94 g, 11.663 mmol, 1.5 equiv) was added and the mixture stirred for 1 h at 0° C. The resulting mixture was quenched with water (100 mL) and extracted with EA (3×100 mL). The combined organics were washed with brine (3×100 mL), dried over anhydrous Na₂SO₄ and concentrated under reduced pressure. The residue was purified by silica gel column chromatography, eluting with PE:EA (1:1) to afford 2-[7-chloroimidazo[1,5-a]pyridin-3-yl]-1-[[2-(trimethylsilyl)ethoxy]methyl]imidazole (1.5 g, 55% yield) as a yellow solid.

LCMS (ES, m/z): [M+H]⁺: 349

### Synthesis of 35-e: 2,4-difluoro-3-[3-(1-[[2-(trimethylsilyl)ethoxy]methyl]imidazol-2-yl)imidazo[1,5-a]pyridin-7-yl]aniline

To a stirred solution of 2-[7-chloroimidazo[1,5-a]pyridin-3-yl]-1-[[2-(trimethylsilyl)ethoxy]methyl]imidazole (1.5 g, 4.3 mmol, 1 equiv), 2,4-difluoro-3-(4,4,5,5-tetramethyl-1,3,2-dioxaborolan-2-yl)aniline (2.19 g, 9 mmol, 2 equiv) K₂CO₃ (1.19 g, 9 mmol, 2 equiv) in dioxane (20 mL) and H₂O (4 mL) were added XPhos (204 mg, 0.4 mmol, 0.1 equiv) and XPhos Pd G3 (363 mg, 0.4 mmol, 0.1 equiv) at room temperature under nitrogen atmosphere. The reaction mixture was stirred for 3 h at 100° C. then cooled. The resulting mixture was diluted with water (100 mL) and extracted with EA (3×50 mL). The combined organics were washed with brine (3×20 mL), dried over anhydrous Na₂SO₄ and concentrated under reduced pressure. The residue was purified by silica gel column chromatography, eluting with PE:EA (5:1) to afford 2,4-difluoro-3-[3-(1-[[2-(trimethylsilyl)ethoxy]methyl]imidazol-2-yl)imidazo[1,5-a]pyridin-7-yl]aniline (1.3 g, 68% yield) as a brown solid.

LCMS (ES, m/z): [M+H]⁺: 442

### Synthesis of 35-f: 5-chloro-N-[2,4-difluoro-3-[3-(1-[[2-(trimethylsilyl)ethoxy]methyl]imidazol-2-yl)imidazo[1,5-a]pyridin-7-yl]phenyl]-2-methoxypyridine-3-sulfonamide

To a stirred solution of 2,4-difluoro-3-[3-(1-[[2-(trimethylsilyl)ethoxy]methyl]imidazol-2-yl)imidazo[1,5-a]pyridin-7-yl]aniline (100 mg, 0.2 mmol, 1 equiv) and pyridine (53 mg, 0.6 mmol, 3 equiv) in DCM (5 mL) was added 5-chloro-2-methoxypyridine-3-sulfonyl chloride (82 mg, 0.3 mmol, 1.5 equiv) in portions at room temperature. The resulting mixture was stirred for 1 h then concentrated under reduced pressure. The residue was purified by silica gel column chromatography, eluting with PE:EA (4:1) to afford 5-chloro-N-[2,4-difluoro-3-[3-(1-[[2-(trimethylsilyl)ethoxy]methyl]imidazol-2-yl)imidazo[1,5-a]pyridin-7-yl]phenyl]-2-methoxypyridine-3-sulfonamide (145 mg, 98% yield) as a yellow solid.

LCMS (ES, m/z): [M+H]⁺: 647

### Synthesis of Compound 35: 5-chloro-N-[2,4-difluoro-3-[3-(1H-imidazol-2-yl)imidazo[1,5-a]pyridin-7-yl]phenyl]-2-methoxypyridine-3-sulfonamide

To a stirred solution of 5-chloro-N-[2,4-difluoro-3-[3-(1-[[2-(trimethylsilyl)ethoxy]methyl]imidazol-2-yl)imidazo[1,5-a]pyridin-7-yl]phenyl]-2-methoxypyridine-3-sulfonamide (150 mg, 0.2 mmol, 1 equiv) in DCM (4 mL) was added TFA (1 mL) dropwise at room temperature. The resulting mixture was stirred for 2 h then concentrated under vacuum. The residue was purified by prep-HPLC with following conditions: Column, welch Vltimate XB-C18, 50×250 mm, 10 μm; Mobile Phase 36-49% MeCN/0.1% aqueous ammonia; Detector, 220 nm; to afford 5-chloro-N-[2,4-difluoro-3-[3-(1H-imidazol-2-yl)imidazo[1,5-a]pyridin-7-yl]phenyl]-2-methoxypyridine-3-sulfonamide (26 mg, 22% yield) as a white solid.

LCMS (ES, m/z): [M+H]⁺: 517

¹H NMR (300 MHz, DMSO-d₆) δ 12.96 (s, 1H), 10.46 (s, 1H), 9.57 (d, J=7.5 Hz, 1H), 8.48 (d, J=2.6 Hz, 1H), 8.07 (d, J=2.6 Hz, 1H), 7.76 (s, 1H), 7.68 (s, 1H), 7.39-7.25 (m, 2H), 7.20 (d, J=11.5 Hz, 2H), 6.83 (d, J=7.5 Hz, 1H), 3.91 (s, 3H).

### Example 51: Synthesis of N-[2,4-difluoro-3-[3-(1H-imidazol-2-yl)imidazo[1,5-a]pyridin-7-yl]phenyl]-5-fluoro-2-methoxypyridine-3-sulfonamide (Compound 36)

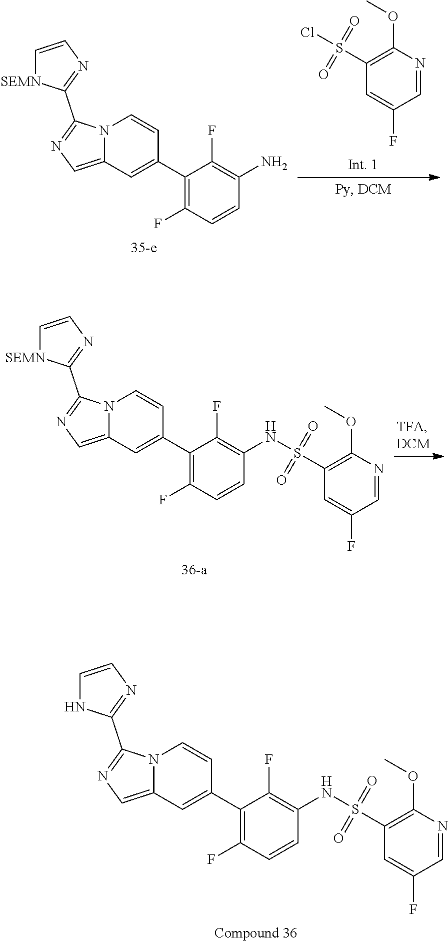 [SMILES: C[Si](C)(C)CCOCn1ccnc1-c1ncc2cc(-c3c(F)ccc(N)c3F)ccn12 ; COc1ncc(F)cc1S(=O)(=O)Cl ; COc1ncc(F)cc1S(=O)(=O)Nc1ccc(F)c(-c2ccn3c(-c4nccn4COCC[Si](C)(C)C)ncc3c2)c1F ; COc1ncc(F)cc1S(=O)(=O)Nc1ccc(F)c(-c2ccn3c(-c4ncc[nH]4)ncc3c2)c1F]

### Synthesis of 36-a: N-[2,4-difluoro-3-[3-(1-[[2-(trimethylsilyl)ethoxy]methyl]imidazol-2-yl)imidazo[1,5-a]pyridin-7-yl]phenyl]-5-fluoro-2-methoxypyridine-3-sulfonamide

To a stirred solution of 2,4-difluoro-3-[3-(1-[[2-(trimethylsilyl)ethoxy]methyl]imidazol-2-yl)imidazo[1,5-a]pyridin-7-yl]aniline (150 mg, 0.3 mmol, 1 equiv) and pyridine (80 mg, 1 mmol, 3 equiv) in DCM (5 mL) was added 5-fluoro-2-methoxypyridine-3-sulfonyl chloride (114 mg, 0.5 mmol, 1.5 equiv) in portions at room temperature. The resulting mixture was stirred for 1 h, then concentrated under reduced pressure. The residue was purified by silica gel column chromatography, eluting with PE:EA (4:1) to afford N-[2,4-difluoro-3-[3-(1-[[2-(trimethylsilyl)ethoxy]methyl]imidazol-2-yl)imidazo[1,5-a]pyridin-7-yl]phenyl]-5-fluoro-2-methoxypyridine-3-sulfonamide (160 mg, 74% yield) as a yellow solid.

LCMS (ES, m/z): [M+H]⁺: 631

### Synthesis of Compound 36: N-[2,4-difluoro-3-[3-(1H-imidazol-2-yl)imidazo[1,5-a]pyridin-7-yl]phenyl]-5-fluoro-2-methoxypyridine-3-sulfonamide

To a stirred solution of N-[2,4-difluoro-3-[3-(1-[[2-(trimethylsilyl)ethoxy]methyl]imidazol-2-yl)imidazo[1,5-a]pyridin-7-yl]phenyl]-5-fluoro-2-methoxypyridine-3-sulfonamide (100 mg, 0.1 mmol, 1 equiv) in DCM (4 mL) was added TFA (1 mL) dropwise at room temperature. The resulting mixture was stirred for 2 h, then concentrated under vacuum. The residue was purified by prep-HPLC with the following conditions: Column, welch Vltimate XB-C18, 50×250 mm, 10 μm; Mobile Phase 32-44% MeCN/0.1% aqueous ammonia; Detector, 220 nm; to afford N-[2,4-difluoro-3-[3-(1H-imidazol-2-yl)imidazo[1,5-a]pyridin-7-yl]phenyl]-5-fluoro-2-methoxypyridine-3-sulfonamide (36 mg, 45% yield) as a yellow solid.

LCMS (ES, m/z): [M+H]⁺: 501

¹H NMR (300 MHz, DMSO-d₆) δ 12.96 (s, 1H), 10.40 (s, 1H), 9.57 (d, J=7.5 Hz, 1H), 8.45 (d, J=3.0 Hz, 1H), 8.01 (dd, J=7.3, 3.0 Hz, 1H), 7.76 (s, 1H), 7.68 (d, J=0.9 Hz, 1H), 7.39-7.28 (m, 2H), 7.20 (d, J=14.0 Hz, 2H), 6.82 (d, J=7.5 Hz, 1H), 3.91 (s, 3H).

### Example 52: Synthesis of 5-chloro-N-[2,4-difluoro-3-[1-(1-methylpyrazol-3-yl)imidazo[1,5-a]pyridin-6-yl]phenyl]-2-methoxypyridine-3-sulfonamide (Compound

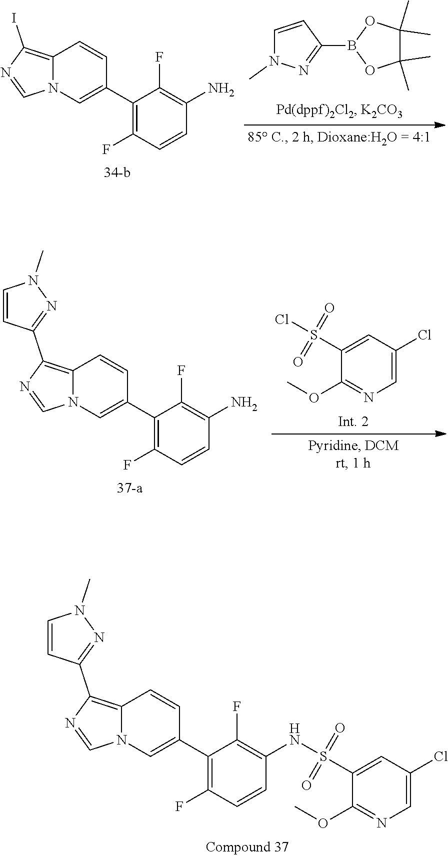

### Synthesis of 37-a: 2,4-difluoro-3-[1-(1-methylpyrazol-3-yl)imidazo[1,5-a]pyridin-6-yl]aniline

Into a 50 mL round-bottom flask was placed 2,4-difluoro-3-[1-iodoimidazo[1,5-a]pyridin-6-yl]aniline (200 mg, 0.5 mmol, 1 equiv), 1-methyl-3-(4,4,5,5-tetramethyl-1,3,2-dioxaborolan-2-yl)pyrazole (168 mg, 0.8 mmol, 1.5 equiv), K₂CO₃ (223 mg, 1.6 mmol, 3 equiv), dioxane (8 mL) and H₂O (2 mL, 0.1 mmol). Pd(dppf)Cl₂ (39 mg, 0.05 mmol, 0.1 equiv) was added in portions at room temperature under N₂ atmosphere. The resulting solution was stirred for 2 h at 85° C. in an oil bath, then cooled and concentrated under vacuum. The residue was purified by silica gel column chromatography, eluting with PE:EA (4:1) to afford 2,4-difluoro-3-[1-(1-methylpyrazol-3-yl)imidazo[1,5-a]pyridin-6-yl]aniline (150 mg, 86% yield) as a brown solid.

LCMS (ES, m/z): [M+H]⁺: 326

### Synthesis of Compound 37: 5-chloro-N-[2,4-difluoro-3-[1-(1-methylpyrazol-3-yl)imidazo[1,5-a]pyridin-6-yl]phenyl]-2-methoxypyridine-3-sulfonamide

Into a 25 mL round-bottom flask was placed 2,4-difluoro-3-[1-(1-methylpyrazol-3-yl)imidazo[1,5-a]pyridin-6-yl]aniline (130 mg, 0.4 mmol, 1 equiv), pyridine (4 mL), 5-chloro-2-methoxypyridine-3-sulfonyl chloride (145 mg, 0.6 mmol, 1.5 equiv) and DCM (1 mL). The resulting solution was stirred for 1 h at room temperature, then concentrated under reduced pressure. The residue was purified by prep-HPLC with the following conditions: Column, welch Vltimate XB-C18, 50×250 mm, 10 μm; Mobile Phase 35-75% MeCN/0.1% aqueous formic acid; to afford 5-chloro-N-[2,4-difluoro-3-[1-(1-methylpyrazol-3-yl)imidazo[1,5-a]pyridin-6-yl]phenyl]-2-methoxypyridine-3-sulfonamide (80 mg, 38% yield) as a light yellow solid.

LCMS (ES, m/z): [M+H]⁺: 531

¹H NMR (300 MHz, DMSO-d₆) δ 8.54-8.42 (m, 3H), 8.17-8.05 (m, 2H), 7.73 (d, J=2.2 Hz, 1H), 7.36 (td, J=8.9, 5.9 Hz, 1H), 7.23 (td, J=9.2, 1.6 Hz, 1H), 6.76 (dt, J=9.4, 1.5 Hz, 1H), 6.62 (d, J=2.2 Hz, 1H), 3.91 (s, 3H), 3.92 (s, 3H).

### Example 53: Synthesis of 5-chloro-N-[2-cyano-4-fluoro-3-[1-(1H-imidazol-2-yl)imidazo[1,5-a]pyridin-6-yl]phenyl]-2-methoxypyridine-3-sulfonamide (Compound

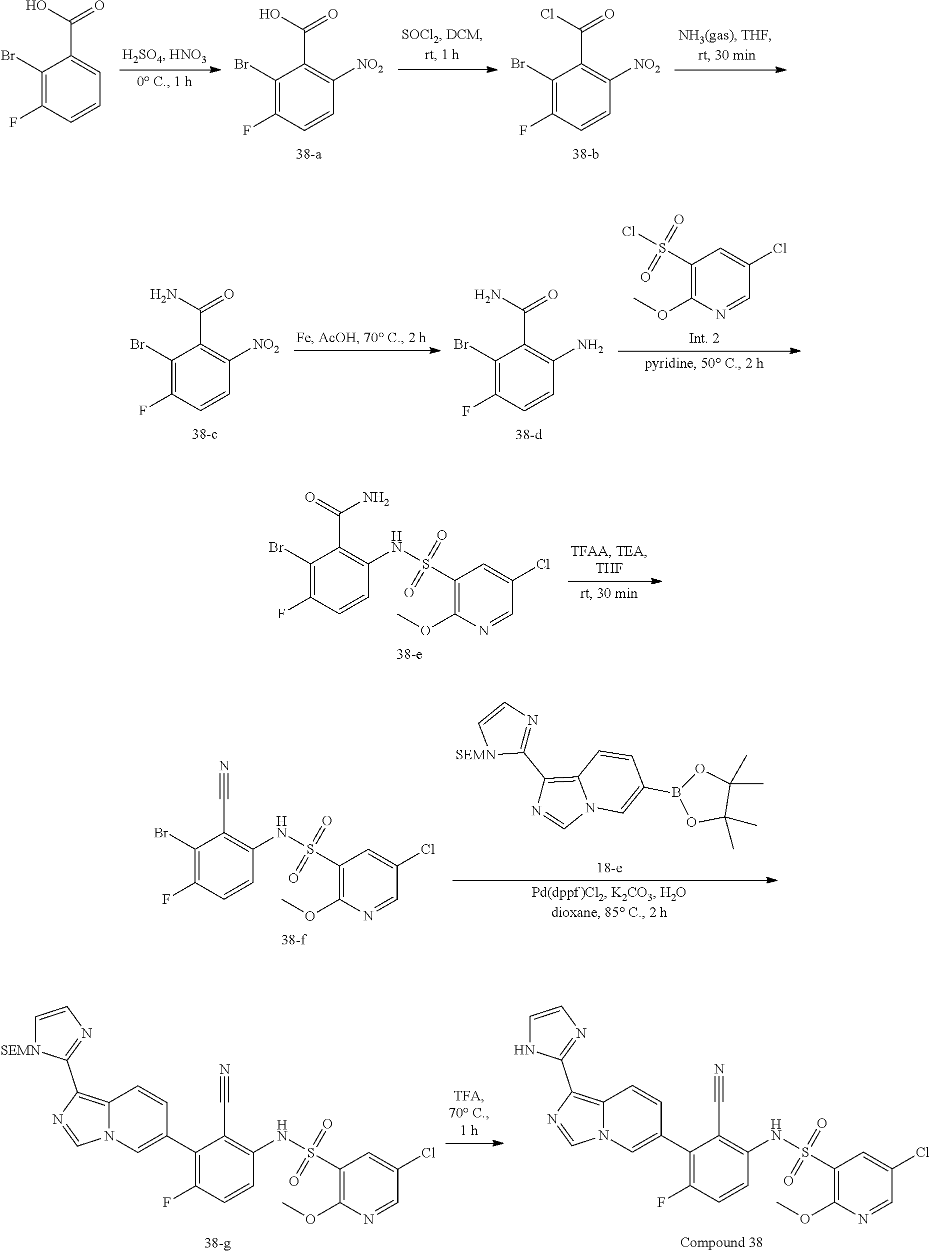

### Synthesis of 38-a: 2-bromo-3-fluoro-6-nitrobenzoic acid

Into a 100 mL 3-necked round-bottom flask was placed 2-bromo-3-fluorobenzoic acid (5 g, 23 mmol, 1 equiv) and H₂SO₄ (30 mL), followed by the addition of HNO₃ (3 mL) dropwise with stirring at 0° C. The resulting solution was stirred for 60 min at 0° C. in an ice/salt bath. The reaction was then quenched by the addition of 100 mL of water/ice, and the resulting solution extracted with 2×70 mL of ethyl acetate. The combined organics were washed with 100 ml of brine, dried over anhydrous sodium sulfate and concentrated under vacuum. 2-Bromo-3-fluoro-6-nitrobenzoic acid (3 g) was isolated as crude yellow solid which was used in next step directly without further purification.

### Synthesis of 38-b: 2-bromo-3-fluoro-6-nitrobenzoyl chloride

Into a 250 mL 3-necked round-bottom flask was placed 2-bromo-3-fluoro-6-nitrobenzoic acid (3 g, 11 mmol, 1 equiv) in DCM (100 mL). This was followed by the addition of SOCl₂ (2.7 g, 23 mmol, 2 equiv) dropwise with stirring at room temperature. The resulting solution was stirred for 60 min at room temperature, then concentrated under vacuum. This gave 2-bromo-3-fluoro-6-nitrobenzoyl chloride (3 g) as a yellow oil which was used in next step directly without further purification.

### Synthesis of 38-c: 2-bromo-3-fluoro-6-nitrobenzamide

Into a 250 mL 3-necked round-bottom flask was placed 2-bromo-3-fluoro-6-nitrobenzoyl chloride (3 g, 11 mmol, 1 equiv) in THE (80 mL). This solution was saturated with ammonia gas at room temperature, then stirred for 30 min. The resulting mixture was concentrated under vacuum to give 2-bromo-3-fluoro-6-nitrobenzamide (3.5 g) as a white solid which was used in next step directly without further purification.

LCMS (ES, m/z): [M+H]⁺: 263

### Synthesis of 38-d: 6-amino-2-bromo-3-fluorobenzamide

Into a 25 mL 3-necked round-bottom flask was placed 2-bromo-3-fluoro-6-nitrobenzamide (1.5 g, 5.7 mmol, 1 equiv), AcOH (20 mL) and iron powder (955 mg, 17 mmol, 3 equiv). The resulting solution was stirred for 2 hr at 90° C. in an oil bath. The solids were removed by filtration and the filtrate concentrated under vacuum. The residue was diluted with 50 mL of H₂O, and the pH adjusted to 10 with ammonia solution. The resulting mixture was extracted with 3×50 mL of dichloromethane and the organic layers combined and dried over anhydrous sodium sulfate. Concentration yielded 6-amino-2-bromo-3-fluorobenzamide (900 mg) as a brown oil which was used in next step directly without further purification.

LCMS (ES, m/z): [M+H]: 233

### Synthesis of 38-e: 2-bromo-6-(5-chloro-2-methoxypyridine-3-sulfonamido)-3-fluorobenzamide

Into a 25 mL 3-necked round-bottom flask was placed 6-amino-2-bromo-3-fluorobenzamide (800 mg, 3.4 mmol, 1 equiv), 5-chloro-2-methoxypyridine-3-sulfonyl chloride (831 mg, 3.4 mmol, 1 equiv) and pyridine (5 mL). The resulting solution was stirred for 60 min at 50° C. in an oil bath. The reaction was quenched by the addition of 50 mL of water and the resulting solution extracted with 4×50 mL of dichloromethane. The combined organics were dried over anhydrous sodium sulfate and concentrated to give 2-bromo-6-(5-chloro-2-methoxypyridine-3-sulfonamido)-3-fluorobenzamide (700 mg) as a yellow oil.

LCMS (ES, m/z): [M+H]⁺:438

### Synthesis of 38-f: N-(3-bromo-2-cyano-4-fluorophenyl)-5-chloro-2-methoxypyridine-3-sulfonamide

Into a 100 mL 3-necked round-bottom flask was placed 2-bromo-6-(5-chloro-2-methoxypyridine-3-sulfonamido)-3-fluorobenzamide (600 mg, 1.4 mmol, 1 equiv), THE (20 mL) and TEA (415 mg, 4.1 mmol, 3 equiv). TFAA (862 mg, 4 mmol, 3 equiv) was added dropwise and the resulting solution was stirred for 30 min at room temperature, before being concentrated under vacuum. The crude product was purified by prep-HPLC with the following conditions: Column, welch Vltimate XB-C18, 50×250 mm, 10 μm; Mobile Phase 50-80% MeCN/0.1% aqueous formic acid; Detector, 220 nm. N-(3-Bromo-2-cyano-4-fluorophenyl)-5-chloro-2-methoxypyridine-3-sulfonamide (240 mg, 40% yield) was obtained as a white solid.

LCMS (ES, m/z): [M+H]⁺: 420

### Synthesis of 38-g: 5-chloro-N-[2-cyano-4-fluoro-3-[1-(1-[[2-(trimethylsilyl)ethoxy]methyl]imidazol-2-yl)imidazo[1,5-a]pyridin-6-yl]phenyl]-2-methoxypyridine-3-sulfonamide

Into a 25 mL 3-necked round-bottom flask, purged and maintained with an inert atmosphere of nitrogen was placed N-(3-bromo-2-cyano-4-fluorophenyl)-5-chloro-2-methoxypyridine-3-sulfonamide (100 mg, 0.24 mmol, 1 equiv), 2-[6-(4,4,5,5-tetramethyl-1,3,2-dioxaborolan-2-yl)imidazo[1,5-a]pyridin-1-yl]-1-[[2-(trimethylsilyl)ethoxy]methyl]imidazole (105 mg, 0.24 mmol, 1 equiv), Pd(dppf)Cl₂ (18 mg, 0.024 mmol, 0.1 equiv), K₂CO₃ (99 mg, 0.7 mmol, 3 equiv), dioxane (5 mL) and H₂O (1 mL). The resulting solution was stirred for 2 hr at 85° C. in an oil bath, then concentrated under vacuum. The residue was applied to a silica gel column, eluting with THF:PE (1:1) to give 5-chloro-N-[2-cyano-4-fluoro-3-[1-(1-[[2-(trimethylsilyl)ethoxy]methyl]imidazol-2-yl)imidazo[1,5-a]pyridin-6-yl]phenyl]-2-methoxypyridine-3-sulfonamide (104 mg, 83% yield) as a yellow oil which was used in next step directly without further purification.

LCMS (ES, m/z): [M+H]: 654

### Synthesis of Compound 38: 5-chloro-N-[2-cyano-4-fluoro-3-[1-(1H-imidazol-2-yl)imidazo[1,5-a]pyridin-6-yl]phenyl]-2-methoxypyridine-3-sulfonamide

Into a 25 mL 3-necked round-bottom flask was placed 5-chloro-N-[2-cyano-4-fluoro-3-[1-(1-[[2-(trimethylsilyl)ethoxy]methyl]imidazol-2-yl)imidazo[1,5-a]pyridin-6-yl]phenyl]-2-methoxypyridine-3-sulfonamide (104 mg, 0.16 mmol, 1 equiv) and TFA (2 mL). The resulting solution was stirred for 60 min at 70° C. in an oil bath, then concentrated under vacuum. The crude product was purified by Flash-Prep-HPLC with the following conditions: welch Vltimate XB-C18, 50×250 mm, 10 μm; Mobile Phase 10-50% MeCN/0.1% aqueous formic acid; Detector, 220 nm. 5-Chloro-N-[2-cyano-4-fluoro-3-[1-(1H-imidazol-2-yl)imidazo[1,5-a]pyridin-6-yl]phenyl]-2-methoxypyridine-3-sulfonamide (5.2 mg, 6% yield) was isolated as a yellow solid.

LCMS (ES, m/z): [M+H]⁺: 524

¹H NMR (300 MHz, Methanol-d₄) δ 8.49 (d, J=8.8 Hz, 2H), 8.32 (d, J=2.6 Hz, 1H), 8.18 (d, J=9.4 Hz, 1H), 8.12 (d, J=2.6 Hz, 1H), 7.61 (dd, J=9.1, 4.7 Hz, 1H), 7.51 (t, J=9.1 Hz, 1H), 7.23 (s, 2H), 7.08-6.99 (m, 1H), 3.99 (s, 3H).

### Example 54: Synthesis of 6-cyano-N-[2,4-difluoro-3-[1-(1H-imidazol-2-yl)imidazo[1,5-a]pyridin-6-yl]phenyl]-1-hydroxy-2,3-dihydro-1H-indene-4-sulfonamide (Compound 39)

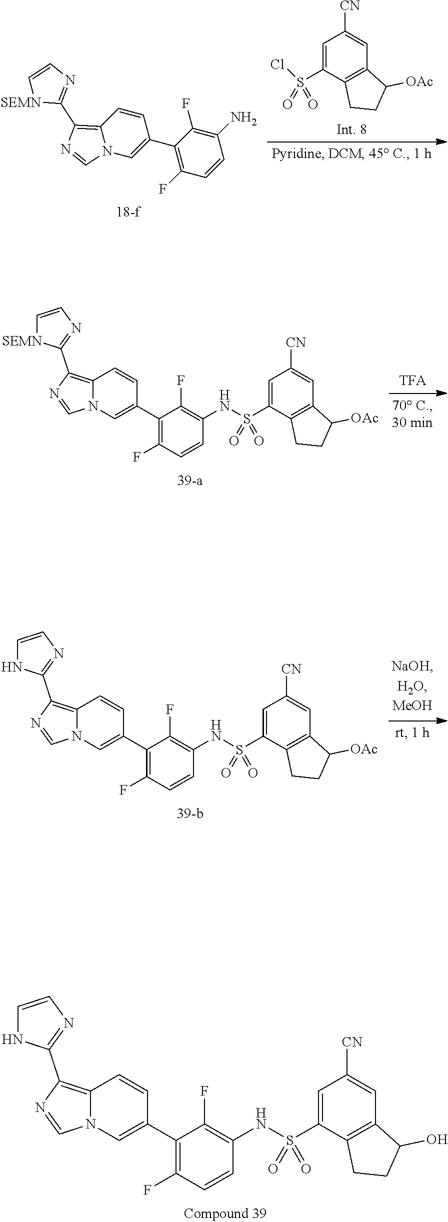 [SMILES: C[Si](C)(C)CCOCn1ccnc1-c1ncn2cc(-c3c(F)ccc(N)c3F)ccc12 ; CC(=O)OC1CCc2c1cc(C#N)cc2S(=O)(=O)Cl ; CC(=O)OC1CCc2c1cc(C#N)cc2S(=O)(=O)Nc1ccc(F)c(-c2ccc3c(-c4nccn4COCC[Si](C)(C)C)ncn3c2)c1F ; CC(=O)OC1CCc2c1cc(C#N)cc2S(=O)(=O)Nc1ccc(F)c(-c2ccc3c(-c4ncc[nH]4)ncn3c2)c1F ; N#Cc1cc2c(c(S(=O)(=O)Nc3ccc(F)c(-c4ccc5c(-c6ncc[nH]6)ncn5c4)c3F)c1)CCC2O]

### Synthesis of 39-a: 6-cyano-4-([2,4-difluoro-3-[1-(1-[[2-(trimethylsilyl)ethoxy]methyl]imidazol-2-yl)imidazo[1,5-a]pyridin-6-yl]phenyl]sulfamoyl)-2,3-dihydro-1H-inden-1-yl acetate

Into a 25 mL 3-necked round-bottom flask was placed 2,4-difluoro-3-[1-(1-[[2-(trimethylsilyl)ethoxy]methyl]imidazol-2-yl)imidazo[1,5-a]pyridin-6-yl]aniline (200 mg, 0.45 mmol, 1 equiv), pyridine (4 mL) and 4-(chlorosulfonyl)-6-cyano-2,3-dihydro-1H-inden-1-yl acetate (136 mg, 0.45 mmol, 1 equiv). The resulting solution was stirred for 1 h at 45° C. in an oil bath, then concentrated under vacuum. The residue was applied to a silica gel column, eluting with THF:PE (1:3). 6-Cyano-4-([2,4-difluoro-3-[1-(1-[[2-(trimethylsilyl)ethoxy]methyl]imidazol-2-yl)imidazo[1,5-a]pyridin-6-yl]phenyl]sulfamoyl)-2,3-dihydro-1H-inden-1-yl acetate (193 mg, 80% yield) was obtained as a yellow oil.

LCMS (ES, m/z): [M+H]⁺: 705

### Synthesis of 39-b: 6-cyano-4-([2,4-difluoro-3-[1-(1H-imidazol-2-yl)imidazo[1,5-a]pyridin-6-yl]phenyl]sulfamoyl)-2,3-dihydro-1H-inden-1-yl acetate

Into a 25 mL 3-necked round-bottom flask was placed 6-cyano-4-([2,4-difluoro-3-[1-(1-[[2-(trimethylsilyl)ethoxy]methyl]imidazol-2-yl)imidazo[1,5-a]pyridin-6-yl]phenyl]sulfamoyl)-2,3-dihydro-1H-inden-1-yl acetate (193 mg, 0.3 mmol, 1 equiv) and TFA (2 mL). The resulting solution was stirred for 30 min at 70° C. in an oil bath, then concentrated under vacuum. The residue was diluted with 20 mL of DCM, and the pH value of the solution was adjusted to 7-8 with saturated NaHCO₃ solution. The organics were dried over anhydrous sodium sulfate, and concentrated under vacuum to give 6-cyano-4-([2,4-difluoro-3-[1-(1H-imidazol-2-yl)imidazo[1,5-a]pyridin-6-yl]phenyl]sulfamoyl)-2,3-dihydro-1H-inden-1-yl acetate (137 mg, 80% yield) as a yellow oil.

LCMS (ES, m/z): [M+H]⁺: 575

### Synthesis of Compound 39: 6-cyano-N-[2,4-difluoro-3-[1-(1H-imidazol-2-yl)imidazo[1,5-a]pyridin-6-yl]phenyl]-1-hydroxy-2,3-dihydro-1H-indene-4-sulfonamide

Into a 25 mL 3-necked round-bottom flask was placed 6-cyano-4-([2,4-difluoro-3-[1-(1H-imidazol-2-yl)imidazo[1,5-a]pyridin-6-yl]phenyl]sulfamoyl)-2,3-dihydro-1H-inden-1-yl acetate (137 mg, 0.24 mmol, 1 equiv) in MeOH (10 mL). NaOH (29 mg, 0.72 mmol, 3 equiv) in H₂O (2 mL) was added dropwise with stirring and the resulting solution was stirred for 30 min at room temperature. The pH was adjusted to 5-6 with 2 M HCl/H₂O and the resulting mixture concentrated under vacuum. The residue was diluted with 15 mL of H₂O and extracted with 3×15 mL of dichloromethane. The combined organics were dried over anhydrous sodium sulfate and concentrated under vacuum. The crude product was purified by Flash-Prep-HPLC with the following conditions: Column, welch Vltimate XB-C18, 50×250 mm, 10 μm; Mobile phase: 20-65% 1:1 ACN:MeOH/aqueous NH₄HCO₃ (10 mmol); Detector, 220 nm. 6-Cyano-N-[2,4-difluoro-3-[1-(1H-imidazol-2-yl)imidazo[1,5-a]pyridin-6-yl]phenyl]-1-hydroxy-2,3-dihydro-1H-indene-4-sulfonamide (33.4 mg, 25% yield) was isolated as a light yellow solid.

LCMS (ES, m/z): [M+H]⁺: 533

¹H NMR (300 MHz, DMSO-d₆) δ 8.48 (d, J=11.0 Hz, 2H), 8.21 (d, J=9.4 Hz, 1H), 7.98-7.90 (m, 2H), 7.28 (td, J=8.9, 5.8 Hz, 1H), 7.15 (t, J=9.3 Hz, 1H), 7.07 (s, 2H), 6.79 (d, J=9.4 Hz, 1H), 5.64 (d, J=5.7 Hz, 1H), 5.10 (q, J=6.5 Hz, 1H), 3.21 (dd, J=8.8, 3.7 Hz, 1H), 2.95 (dt, J=17.5, 8.0 Hz, 1H), 2.38 (d, J=13.1 Hz, 1H), 1.78 (dd, J=13.1, 6.9 Hz, 1H).

### Example 55: Synthesis of 5-cyano-N-[2,4-difluoro-3-[3-(1H-imidazol-2-yl)imidazo[1,5-a]pyridin-7-yl]phenyl]-2-methoxypyridine-3-sulfonamide (Compound 40)

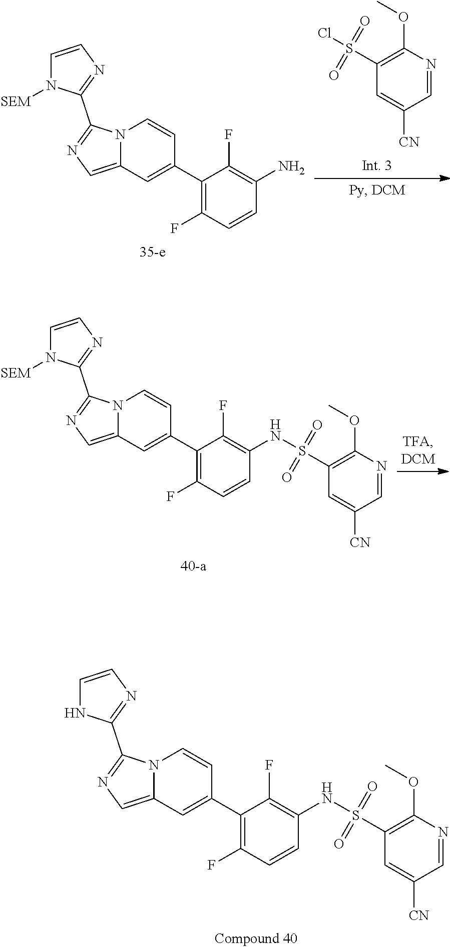 [SMILES: C[Si](C)(C)CCOCn1ccnc1-c1ncc2cc(-c3c(F)ccc(N)c3F)ccn12 ; COc1ncc(C#N)cc1S(=O)(=O)Cl ; COc1ncc(C#N)cc1S(=O)(=O)Nc1ccc(F)c(-c2ccn3c(-c4nccn4COCC[Si](C)(C)C)ncc3c2)c1F ; COc1ncc(C#N)cc1S(=O)(=O)Nc1ccc(F)c(-c2ccn3c(-c4ncc[nH]4)ncc3c2)c1F]

### Synthesis of 40-a: 5-cyano-N-[2,4-difluoro-3-[3-(1-[[2-(trimethylsilyl)ethoxy]methyl]imidazol-2-yl)imidazo[1,5-a]pyridin-7-yl]phenyl]-2-methoxypyridine-3-sulfonamide

To a solution of 2,4-difluoro-3-[3-(1-[[2-(trimethylsilyl)ethoxy]methyl]imidazol-2-yl)imidazo[1,5-a]pyridin-7-yl]aniline (100 mg, 0.2 mmol, 1 equiv) in DCM (5 mL) were added pyridine (53 mg, 0.6 mmol, 3 equiv) and 5-cyano-2-methoxypyridine-3-sulfonyl chloride (79 mg, 0.3 mmol, 1.5 equiv). The resulting solution was stirred at room temperature for 3 hours, then concentrated to give the 5-cyano-N-[2,4-difluoro-3-[3-(1-[[2-(trimethylsilyl)ethoxy]methyl]imidazol-2-yl)imidazo[1,5-a]pyridin-7-yl]phenyl]-2-methoxypyridine-3-sulfonamide (120 mg, 83% yield) as a brown solid.

LCMS (ES, m/z): [M+H]⁺: 638

### Synthesis of Compound 40: 5-cyano-N-[2,4-difluoro-3-[3-(1H-imidazol-2-yl)imidazo[1,5-a]pyridin-7-yl]phenyl]-2-methoxypyridine-3-sulfonamide

To a stirred solution of 5-cyano-N-[2,4-difluoro-3-[3-(1-[[2-(trimethylsilyl)ethoxy]methyl]imidazol-2-yl)imidazo[1,5-a]pyridin-7-yl]phenyl]-2-methoxypyridine-3-sulfonamide (60 mg, 1 equiv) in DCM (4 mL) was added TFA (1 mL) dropwise at room temperature. The resulting mixture was stirred for 1 h then concentrated.

The residue was purified by prep-HPLC with the following conditions: Column, welch Vltimate XB-C18, 50×250 mm, 10 μm; Mobile Phase 36-60% MeCN/1% aqueous NH₃; Detector 220 nm; to afford 5-cyano-N-[2,4-difluoro-3-[3-(1H-imidazol-2-yl)imidazo[1,5-a]pyridin-7-yl]phenyl]-2-methoxypyridine-3-sulfonamide (40 mg, 50% yield) as a white solid.

LCMS (ES, m/z): [M+H]⁺: 508 H NMR (300 MHz, DMSO-d₆) δ 12.96 (s, 1H), 10.51 (s, 1H), 9.57 (d, J=7.5 Hz, 1H), 8.94 (d, J=2.2 Hz, 1H), 8.50 (d, J=2.2 Hz, 1H), 7.76 (s, 1H), 7.69 (s, 1H), 7.42-7.15 (m, 4H), 6.82 (dd, J=7.5, 1.6 Hz, 1H), 4.03 (s, 3H)

### Example 56: Synthesis of 5-chloro-N-[2,4-difluoro-3-[8-fluoro-3-(1H-imidazol-2-yl)imidazo[1,5-a]pyridin-7-yl]phenyl]-2-methoxypyridine-3-sulfonamide (Compound

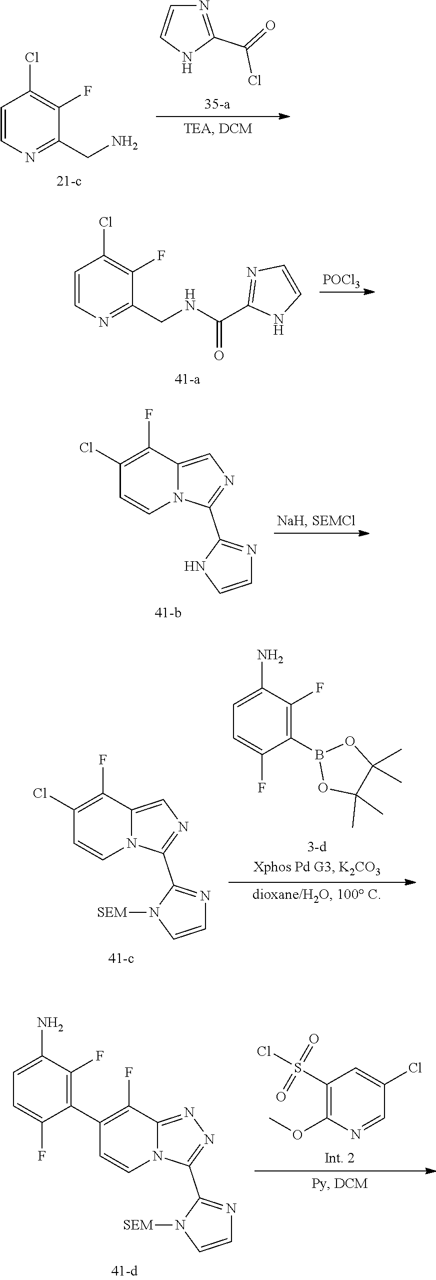

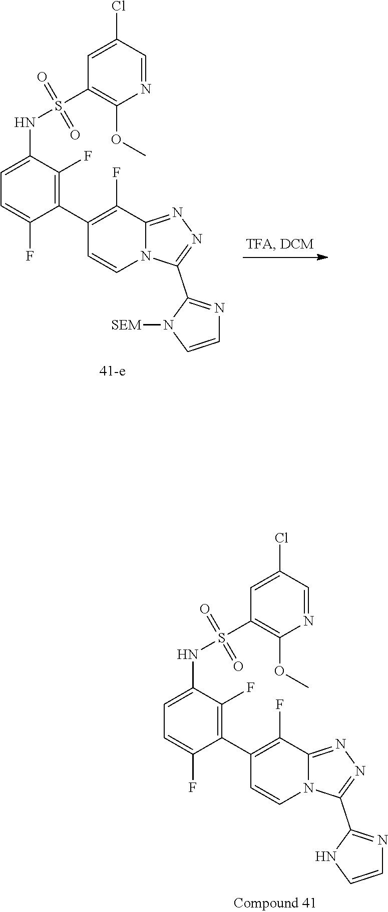 [SMILES: COc1ncc(Cl)cc1S(=O)(=O)Nc1ccc(F)c(-c2ccn3c(-c4nccn4COCC[Si](C)(C)C)nnc3c2F)c1F ; COc1ncc(Cl)cc1S(=O)(=O)Nc1ccc(F)c(-c2ccn3c(-c4ncc[nH]4)nnc3c2F)c1F]

### Synthesis of 41-a: N-[(4-chloro-3-fluoropyridin-2-yl)methyl]-1H-imidazole-2-carboxamide

To a stirred solution of 1-(4-chloro-3-fluoropyridin-2-yl)methanamine (3.5 g, 21 mmol, 1 equiv) and TEA (4.4 g, 43 mmol, 2 equiv) in DCM (50 mL) was added a solution of 1H-imidazole-2-carbonyl chloride (3.13 g, 23 mmol, 1.1 equiv) in DCM (10 mL) at 0° C. The resulting solution was stirred for 1 h at room temperature. The reaction mixture was diluted with water (100 mL) and extracted with DCM (3×300 mL). The combined organics were washed with brine (2×20 mL), dried over anhydrous Na₂SO₄ and concentrated under reduced pressure to afford N-[(4-chloro-3-fluoropyridin-2-yl)methyl]-1H-imidazole-2-carboxamide (4 g, crude) as a yellow solid which was used in the next step without further purification.

LCMS (ES, m/z): [M+H]⁺: 255

### Synthesis of 41-b: 2-[7-chloro-8-fluoroimidazo[1,5-a]pyridin-3-yl]-1H-imidazole

N-[(4-chloro-3-fluoropyridin-2-yl)methyl]-1H-imidazole-2-carboxamide (3 g, 12 mmol, 1 equiv) was dissolved in POCl₃ (20 mL) and stirred for 5 h at 110° C. The mixture was allowed to cool and was concentrated under vacuum. The residue was carefully quenched by addition of saturated aqueous NaHCO₃ (100 mL). The resulting mixture was extracted with EA (3×100 mL), and the combined organics were washed with brine (3×50 mL), dried over anhydrous Na₂SO₄, then concentrated under reduced pressure. The residue was purified by silica gel column chromatography, eluting with PE:EA (1:1) to afford 2-[7-chloro-8-fluoroimidazo[1,5-a]pyridin-3-yl]-1H-imidazole (900 mg, 32% yield) as a yellow solid.

LCMS (ES, m/z): [M+H]⁺: 237

### Synthesis of 41-c: 2-[7-chloro-8-fluoroimidazo[1,5-a]pyridin-3-yl]-1-[[2-(trimethylsilyl)ethoxy]methyl]imidazole

To a stirred solution of 2-[7-chloro-8-fluoroimidazo[1,5-a]pyridin-3-yl]-1H-imidazole (900 mg, 3.8 mmol, 1 equiv) in THE (20 mL) was added NaH (304 mg, 7.6 mmol, 2 equiv, 60% in oil) in portions at 0° C. The resulting mixture was stirred for 1 h at 0° C., then SEM-C1 (951 mg, 5.7 mmol, 1.5 equiv) was added and the mixture stirred in an ice bath for 1 h. The reaction was quenched with water (50 mL) at 0° C., and the resulting mixture was extracted with EA (3×100 mL). The combined organics were washed with brine (3×20 mL), dried over anhydrous Na₂SO₄ and concentrated under reduced pressure. The residue was purified by silica gel column chromatography, eluting with PE:EA (10:1) to afford 2-[7-chloro-8-fluoroimidazo[1,5-a]pyridin-3-yl]-1-[[2-(trimethylsilyl)ethoxy]methyl]imidazole (1 g, 71% yield) as a yellow solid.

LCMS (ES, m/z): [M+H]⁺: 367

### Synthesis of 41-d: 2,4-difluoro-3-[8-fluoro-3-(1-[[2-(trimethylsilyl)ethoxy]methyl]imidazol-2-yl)imidazo[1,5-a]pyridin-7-yl]aniline

To a solution of 2-[7-chloro-8-fluoroimidazo[1,5-a]pyridin-3-yl]-1-[[2-(trimethylsilyl)ethoxy]methyl]imidazole (1.4 g, 3.8 mmol, 1 equiv), 2,4-difluoro-3-(4,4,5,5-tetramethyl-1,3,2-dioxaborolan-2-yl)aniline (973 mg, 3.8 mmol, 1 equiv), and K₂CO₃ (1 g, 7.6 mmol, 2 equiv) in dioxane (20 mL) and H₂O (5 mL) were added XPhos (181 mg, 0.38 mmol, 0.1 equiv) and XPhos Pd G3 (322 mg, 0.38 mmol, 0.1 equiv) at room temperature under nitrogen atmosphere. The resulting solution was stirred at 100° C. under nitrogen atmosphere for 5 h. After cooling to room temperature, water (100 mL) was added and the mixture extracted with ethyl acetate (3×200 mL). The combined organics were washed with brine (2×30 mL), dried over anhydrous Na₂SO₄ and concentrated under reduced pressure to give a crude product. This was purified by column chromatography over silica gel (eluent: PE:EA=4:1) to afford 2,4-difluoro-3-[8-fluoro-3-(1-[[2-(trimethylsilyl)ethoxy]methyl]imidazol-2-yl)imidazo[1,5-a]pyridin-7-yl]aniline (800 mg, 45% yield) as a yellow oil.

LCMS (ES, m/z): [M+H]⁺: 460

### Synthesis of 41-e: 5-chloro-N-(2,4-difluoro-3-(8-fluoro-3-(1-((2-(trimethylsilyl)ethoxy)methyl)-1H-imidazol-2-yl)imidazo[1,5-a]pyridin-7-yl)phenyl)-2-methoxypyridine-3-sulfonamide

To a solution of 2,4-difluoro-3-[8-fluoro-3-(1-[[2-(trimethylsilyl)ethoxy]methyl]imidazol-2-yl)imidazo[1,5-a]pyridin-7-yl]aniline (150 mg, 0.3 mmol, 1 equiv) and pyridine (77 mg, 0.9 mmol, 3.00 equiv) in DCM (5 mL) was added 5-chloro-2-methoxypyridine-3-sulfonyl chloride (94 mg, 0.39 mmol, 1.2 equiv) in portions at room temperature. The resulting solution was stirred for 1 h at room temperature. The resulting mixture was concentrated under reduced pressure and the residue purified by column chromatography over silica gel (eluent: PE:EA=5:1) to afford 5-chloro-N-[2,4-difluoro-3-[8-fluoro-3-(1-[[2-(trimethylsilyl)ethoxy]methyl]imidazol-2-yl)imidazo[1,5-a]pyridin-7-yl]phenyl]-2-methoxypyridine-3-sulfonamide (100 mg, 46% yield) as a colorless oil.

LCMS (ES, m/z): [M+H]⁺: 665

### Synthesis of Compound 41: 5-chloro-N-[2,4-difluoro-3-[8-fluoro-3-(1H-imidazol-2-yl)imidazo[1,5-a]pyridin-7-yl]phenyl]-2-methoxypyridine-3-sulfonamide

To a stirred solution of 5-chloro-N-[2,4-difluoro-3-[8-fluoro-3-(1-[[2-(trimethylsilyl)ethoxy]methyl]imidazol-2-yl)imidazo[1,5-a]pyridin-7-yl]phenyl]-2-methoxypyridine-3-sulfonamide (70 mg, 0.1 mmol, 1 equiv) in DCM (4 mL) was added TFA (1 mL) dropwise at room temperature. The resulting mixture was stirred for 1 h at room temperature, then concentrated under reduced pressure. The residue was purified by prep-HPLC with the following conditions: Column, welch Vltimate XB-C18, 50×250 mm, 10 m; Mobile Phase: 35-65% MeCN/0.1% aqueous formic acid; Detector, 220 nm; to afford 5-chloro-N-[2,4-difluoro-3-[8-fluoro-3-(1H-imidazol-2-yl)imidazo[1,5-a]pyridin-7-yl]phenyl]-2-methoxypyridine-3-sulfonamide (40 mg, 71% yield) as a white solid.

LCMS (ES, m/z): [M+H]⁺: 535

¹H NMR (300 MHz, DMSO-d₆) δ 13.11 (s, 1H), 10.47 (s, 1H), 9.44 (d, J=7.4 Hz, 1H), 8.52 (d, J=2.6 Hz, 1H), 8.08 (d, J=2.6 Hz, 1H), 7.87 (s, 1H), 7.46 (td, J=8.9, 5.8 Hz, 1H), 7.38-7.15 (m, 3H), 6.85 (t, J=6.9 Hz, 1H), 3.91 (s, 3H).

### Example 57: Synthesis of 5-cyano-N-[2,4-difluoro-3-[8-fluoro-3-(1H-imidazol-2-yl)imidazo[1,5-a]pyridin-7-yl]phenyl]-2-methoxypyridine-3-sulfonamide (Compound 42)

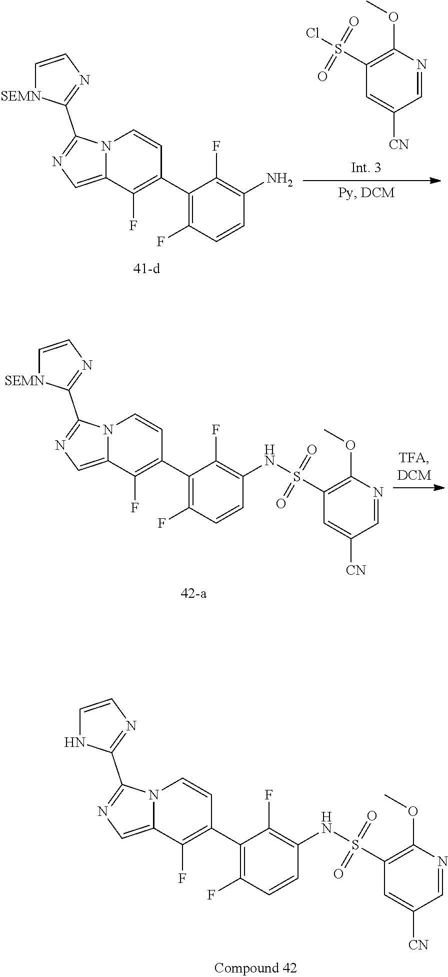 [SMILES: C[Si](C)(C)CCOCn1ccnc1-c1ncc2c(F)c(-c3c(F)ccc(N)c3F)ccn12 ; COc1ncc(C#N)cc1S(=O)(=O)Cl ; COc1ncc(C#N)cc1S(=O)(=O)Nc1ccc(F)c(-c2ccn3c(-c4nccn4COCC[Si](C)(C)C)ncc3c2F)c1F ; COc1ncc(C#N)cc1S(=O)(=O)Nc1ccc(F)c(-c2ccn3c(-c4ncc[nH]4)ncc3c2F)c1F]

### Synthesis of 42-a: 5-cyano-N-[2,4-difluoro-3-[8-fluoro-3-(1-[[2-(trimethylsilyl)ethoxy]methyl]imidazol-2-yl)imidazo[1,5-a]pyridin-7-yl]phenyl]-2-methoxypyridine-3-sulfonamide

To a solution of 2,4-difluoro-3-[8-fluoro-3-(1-[[2-(trimethylsilyl)ethoxy]methyl]imidazol-2-yl)imidazo[1,5-a]pyridin-7-yl]aniline (150 mg, 0.3 mmol, 1 equiv) and pyridine (77 mg, 0.9 mmol, 3 equiv) in DCM (5 mL) was added 5-cyano-2-methoxypyridine-3-sulfonyl chloride (91 mg, 0.3 mmol, 1.2 equiv) in portions at room temperature. The resulting solution was stirred for 1 h at room temperature. The mixture was concentrated and the residue purified by column chromatography over silica gel (eluent: PE:EA=4:1) to afford 5-cyano-N-[2,4-difluoro-3-[8-fluoro-3-(1-[[2-(trimethylsilyl)ethoxy]methyl]imidazol-2-yl)imidazo[1,5-a]pyridin-7-yl]phenyl]-2-methoxypyridine-3-sulfonamide (100 mg, 46% yield) as a yellow solid.

### Synthesis of Compound 42: 5-cyano-N-[2,4-difluoro-3-[8-fluoro-3-(1H-imidazol-2-yl)imidazo[1,5-a]pyridin-7-yl]phenyl]-2-methoxypyridine-3-sulfonamide

To a stirred solution of 5-cyano-N-[2,4-difluoro-3-[8-fluoro-3-(1-[[2-(trimethylsilyl)ethoxy]methyl]imidazol-2-yl)imidazo[1,5-a]pyridin-7-yl]phenyl]-2-methoxypyridine-3-sulfonamide (80 mg) in DCM (4 mL) was added TFA (1 mL) dropwise at room temperature. The resulting mixture was stirred for 1 h at room temperature then concentrated under reduced pressure. The residue was purified by prep-HPLC with following conditions: Column, welch Vltimate XB-C18, 50×250 mm, 10 m; Mobile Phase 25-60% MeCN/0.1% aqueous formic acid; Detector 220 nm; to afford 5-cyano-N-[2,4-difluoro-3-[8-fluoro-3-(1H-imidazol-2-yl)imidazo[1,5-a]pyridin-7-yl]phenyl]-2-methoxypyridine-3-sulfonamide (30 mg, 46% yield) as a white solid.

LCMS (ES, m/z): [M+H]⁺: 526

¹H NMR (300 MHz, DMSO-d₆) δ 13.11 (s, 1H), 10.56 (s, 1H), 9.43 (d, J=7.3 Hz, 1H), 8.94 (d, J=2.2 Hz, 1H), 8.49 (d, J=2.3 Hz, 1H), 7.86 (s, 1H), 7.46 (td, J=9.0, 5.9 Hz, 1H), 7.33-7.14 (m, 2H), 6.85 (t, J=6.9 Hz, 1H), 4.01 (s, 3H).

### Example 58: Synthesis of N-[3-[1-(4-cyano-1H-imidazol-2-yl)imidazo[1,5-a]pyridin-6-yl]-2,4-difluorophenyl]-5-chloro-2-methoxypyridine-3-sulfonamide (Compound 43)

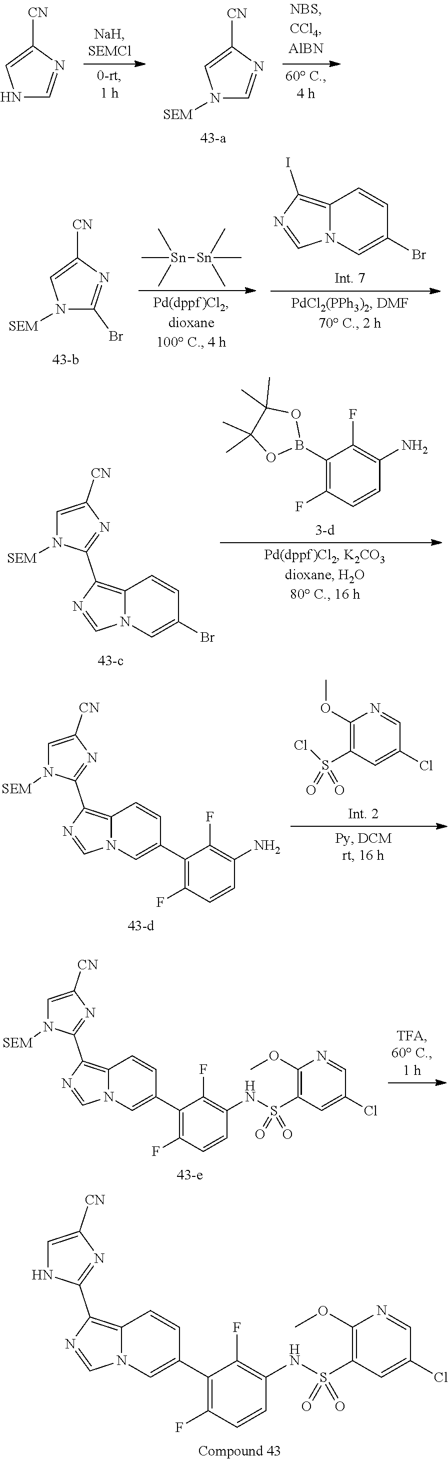

### Synthesis of 43-a: 1-[[2-(trimethylsilyl)ethoxy]methyl]imidazole-4-carbonitrile

Into a 100 mL 3-necked round-bottom flask were added 1H-imidazole-4-carbonitrile (2 g, 21.4 mmol, 1 equiv) in DMF (20 mL). To the above mixture was added 60% NaH (1.55 g, 64.4 mmol, 3 equiv) in portions at 0° C. The resulting mixture was stirred for 30 min at 0° C., and [2-(chloromethoxy)ethyl]trimethylsilane (3.9 g, 23.6 mmol, 1.1 equiv) was added dropwise at 0° C. The reaction was stirred for 1 h at room temperature, then quenched with water (100 ml). The resulting mixture was diluted with more water (100 mL), and extracted with EtOAc (3×100 mL). The combined organics were washed with brine (3×50 mL), dried over anhydrous Na₂SO₄ and concentrated under reduced pressure. The residue was purified by silica gel column chromatography, with PE/EtOAc (1:1) to afford 1-[[2-(trimethylsilyl)ethoxy]methyl]imidazole-4-carbonitrile (3.2 g, 67% yield) as a colorless oil.

LCMS (ES, m/z): [M+H]⁺: 224.

### Synthesis of 43-b: 2-bromo-1-[[2-(trimethylsilyl)ethoxy]methyl]imidazole-4-carbonitrile

Into a 250 mL round-bottom flask were added 1-[[2-(trimethylsilyl)ethoxy]methyl]imidazole-4-carbonitrile (3.2 g, 14.3 mmol, 1 equiv), NBS (2.8 g, 15.7 mmol, 1.1 equiv), CCl₄ (80 mL) and AIBN (0.24 g, 1.4 mmol, 0.1 equiv). The resulting mixture was stirred for 4 h at 60° C. The resulting mixture was concentrated under reduced pressure, and the residue purified by silica gel column chromatography, eluting with PE/EtOAc (1:1) to afford 2-bromo-1-[[2-(trimethylsilyl)ethoxy]methyl]imidazole-4-carbonitrile (3.7 g, 85% yield) as a colorless oil.

LCMS (ES, m/z): [M+H]⁺: 302, 304.

### Synthesis of 43-c: 2-[6-bromoimidazo[1,5-a]pyridin-1-yl]-1-[[2-(trimethylsilyl)ethoxy]methyl]imidazole-4-carbonitrile

Into a 40 mL vial were added 2-bromo-1-[[2-(trimethylsilyl)ethoxy]methyl]imidazole-4-carbonitrile (2.8 g, 9.2 mmol, 1 equiv), hexamethyldistannane (3.37 g, 10.3 mmol, 1.1 equiv), Pd(dppf)Cl₂ (0.68 g, 0.9 mmol, 0.1 equiv) and dioxane (10 mL). The resulting mixture was stirred for 4 h at 100° C. under nitrogen atmosphere, then concentrated under reduced pressure. To the residue was added Pd(PPh₃)₂Cl₂ (0.65 g, 0.9 mmol, 0.1 equiv), 6-bromo-1-iodoimidazo[1,5-a]pyridine (1 g, 3.1 mmol, 0.34 equiv) and DMF (1 mL). The resulting mixture was stirred for 16 h at 100° C. under nitrogen atmosphere. The mixture was allowed to cool to room temperature and was diluted with water (100 mL). This was extracted with EtOAc (3×100 mL), and the combined organics were washed with brine (3×100 mL), dried over anhydrous Na₂SO₄ and concentrated under reduced pressure. The residue was purified by silica gel column chromatography, eluting with PE/EtOAc (3:1) to afford 2-[6-bromoimidazo[1,5-a]pyridin-1-yl]-1-[[2-(trimethylsilyl)ethoxy]methyl]imidazole-4-carbonitrile (210 mg, 5.4% yield) as a brown solid.

LCMS (ES, m/z): [M+H]⁺: 418, 420.

### Synthesis of 43-d: 2-[6-(3-amino-2,6-difluorophenyl)imidazo[1,5-a]pyridin-1-yl]-1-[[2-(trimethylsilyl) ethoxy]methyl]imidazole-4-carbonitrile

Into a 40 mL vial were added 2-[6-bromoimidazo[1,5-a]pyridin-1-yl]-1-[[2-(trimethylsilyl)ethoxy]methyl]imidazole-4-carbonitrile (220 mg, 0.5 mmol, 1 equiv), 2,4-difluoro-3-(4,4,5,5-tetramethyl-1,3,2-dioxaborolan-2-yl)aniline (395 mg, 1.5 mmol, 3.0 equiv), Pd(dppf)Cl₂ (42 mg, 0.06 mmol, 0.11 equiv), K₂CO₃ (220 mg, 1.6 mmol, 3 equiv), dioxane (5 mL) and H₂O (0.5 mL). The resulting mixture was stirred for 16 h at 80° C. under nitrogen atmosphere, then was diluted with water (100 mL). This was extracted with EtOAc (3×100 mL), and the combined organics were washed with brine (3×50 mL), dried over anhydrous Na₂SO₄ and concentrated under reduced pressure. The residue was purified by silica gel column chromatography, eluting with PE/EtOAc (3:2) to afford 2-[6-(3-amino-2,6-difluorophenyl)imidazo[1,5-a]pyridin-1-yl]-1-[[2-(trimethylsilyl)ethoxy]methyl]imidazole-4-carbonitrile (125 mg, 51% yield) as a light brown solid.

LCMS (ES, m/z): [M+H]⁺: 467.

### Synthesis of 43-e: 5-chloro-N-[3-[1-(4-cyano-1-[[2-(trimethylsilyl)ethoxy]methyl]imidazol-2-yl) imidazo[1,5-a]pyridin-6-yl]-2,4-difluorophenyl]-2-methoxypyridine-3-sulfonamide

Into an 8 mL vial were added 2-[6-(3-amino-2,6-difluorophenyl)imidazo[1,5-a]pyridin-1-yl]-1-[[2-(trimethylsilyl)ethoxy]methyl]imidazole-4-carbonitrile (50 mg, 0.24 mmol, 1 equiv), 5-chloro-2-methoxypyridine-3-sulfonyl chloride (39 mg, 0.16 mmol, 1.5 equiv), pyridine (40 mg, 0.51 mmol, 4.7 equiv) and DCM (2 mL). The resulting mixture was stirred overnight at room temperature, then was concentrated under reduced pressure. The residue was purified by silica gel column chromatography, eluting with PE/EtOAc (3:2) to afford 5-chloro-N-[3-[1-(4-cyano-1-[[2-(trimethylsilyl)ethoxy]methyl]imidazol-2-yl)imidazo[1,5-a]pyridin-6-yl]-2,4-difluorophenyl]-2-methoxypyridine-3-sulfonamide (52 mg, 33% yield) as a light yellow solid.

LCMS (ES, m/z): [M+H]⁺: 672.

### Synthesis of Compound 43: N-[3-[1-(4-cyano-1H-imidazol-2-yl)imidazo[1,5-a]pyridin-6-yl]-2,4-difluorophenyl]-5-chloro-2-methoxypyridine-3-sulfonamide

Into an 8 mL vial were added 5-chloro-N-[3-[1-(4-cyano-1-[[2-(trimethylsilyl)ethoxy]methyl]imidazol-2-yl)imidazo[1,5-a]pyridin-6-yl]-2,4-difluorophenyl]-2-methoxypyridine-3-sulfonamide (47 mg, 0.07 mmol, 1 equiv) and TFA (2 mL). The resulting mixture was stirred for 1 h at 60° C., then was concentrated under vacuum. The crude product was purified by Prep-HPLC with the following conditions: Column: Atlantis HILIC OBD, 19*150 mm*5 μm; Mobile Phase: 30-65% MeCN/0.1% aqueous formic acid; Flow rate: 90 mL/min; to afford N-[3-[1-(4-cyano-1H-imidazol-2-yl)imidazo[1,5-a]pyridin-6-yl]-2,4-difluorophenyl]-5-chloro-2-methoxypyridine-3-sulfonamide (13.5 mg, 42% yield) as a white solid.

LCMS (ES, m/z): [M+H]⁺: 542.

¹H NMR (300 MHz, DMSO-d₆) δ 13.40 (s, 1H), 10.46 (s, 1H), 8.60 (m, 2H), 8.52 (d, J=2.6 Hz, 1H), 8.20 (d, J=9.4 Hz, 1H), 8.13 (d, J=2.5 Hz, 1H), 8.09 (d, J=2.6 Hz, 1H), 7.44-7.34 (m, 1H), 7.27 (t, J=9.3 Hz, 1H), 6.97 (d, J=9.5 Hz, 1H), 3.93 (s, 3H).

### Example 59: Synthesis of N-[3-[1-(4-cyano-1H-imidazol-2-yl)imidazo[1,5-a]pyridin-6-yl]-2,4-difluorophenyl]-5-fluoro-2-methylpyridine-3-sulfonamide (Compound 44)

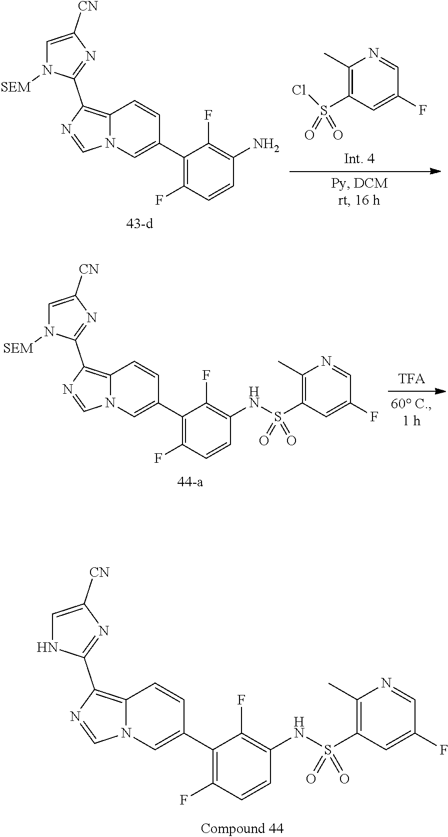 [SMILES: C[Si](C)(C)CCOCn1cc(C#N)nc1-c1ncn2cc(-c3c(F)ccc(N)c3F)ccc12 ; Cc1ncc(F)cc1S(=O)(=O)Cl ; Cc1ncc(F)cc1S(=O)(=O)Nc1ccc(F)c(-c2ccc3c(-c4nc(C#N)cn4COCC[Si](C)(C)C)ncn3c2)c1F ; Cc1ncc(F)cc1S(=O)(=O)Nc1ccc(F)c(-c2ccc3c(-c4nc(C#N)c[nH]4)ncn3c2)c1F]

### Synthesis of 44-a: N-[3-[1-(4-cyano-1-[[2-(trimethylsilyl)ethoxy]methyl]imidazol-2-yl)imidazo[1,5-a]pyridin-6-yl]-2,4-difluorophenyl]-5-fluoro-2-methylpyridine-3-sulfonamide

Into an 8 mL vial were added 2-[6-(3-amino-2,6-difluorophenyl)imidazo[1,5-a]pyridin-1-yl]-1-[[2-(trimethylsilyl)ethoxy]methyl]imidazole-4-carbonitrile (50 mg, 0.11 mmol, 1 equiv), 5-fluoro-2-methylpyridine-3-sulfonyl chloride (67 mg, 0.32 mmol, 3 equiv), pyridine (42 mg, 0.53 mmol, 5 equiv) and DCM (2 mL). The resulting mixture was stirred overnight at room temperature. The mixture was concentrated under reduced pressure and the residue was purified by silica gel column chromatography, eluting with PE/EtOAc (3:2) to afford N-[3-[1-(4-cyano-1-[[2-(trimethylsilyl)ethoxy]methyl]imidazol-2-yl)imidazo[1,5-a]pyridin-6-yl]-2,4-difluorophenyl]-5-fluoro-2-methylpyridine-3-sulfonamide (45 mg, 66% yield) as a light yellow solid.

LCMS (ES, m/z): [M−H]⁻: 640.

### Synthesis of Compound 44: N-[3-[1-(4-cyano-1H-imidazol-2-yl)imidazo[1,5-a]pyridin-6-yl]-2,4-difluorophenyl]-5-fluoro-2-methylpyridine-3-sulfonamide

Into an 8 mL vial were added N-[3-[1-(4-cyano-1-[[2-(trimethylsilyl)ethoxy]methyl]imidazol-2-yl)imidazo[1,5-a]pyridin-6-yl]-2,4-difluorophenyl]-5-fluoro-2-methylpyridine-3-sulfonamide (40 mg, 0.06 mmol, 1 equiv) and TFA (2 mL). The resulting mixture was stirred for 1 h at 60° C., then concentrated under vacuum. The crude product was purified by Prep-HPLC with the following conditions: Column: Atlantis HILIC OBD, 19*150 mm*5 μm; Mobile Phase 25-65% MeCN/0.1% aqueous formic acid; Flow rate: 90 mL/min; Detector 220 nm; to afford N-[3-[1-(4-cyano-1H-imidazol-2-yl)imidazo[1,5-a]pyridin-6-yl]-2,4-difluorophenyl]-5-fluoro-2-methylpyridine-3-sulfonamide (13.5 mg, 42% yield) as a white solid.

LCMS (ES, m/z): [M+H]⁺: 510.

¹H NMR (300 MHz, DMSO-d₆) δ 13.40 (s, 1H), 10.79 (s, 1H), 8.75 (d, J=2.8 Hz, 1H), 8.59 (s, 2H), 8.19 (d, J=9.5 Hz, 1H), 8.13 (d, J=2.5 Hz, 1H), 7.96 (dd, J=8.2, 2.8 Hz, 1H), 7.37 (td, J=8.9, 5.9 Hz, 1H), 7.27 (dd, J=9.9, 8.5 Hz, 1H), 6.93 (d, J=9.4 Hz, 1H), 2.78 (d, J=1.2 Hz, 3H).

### Example 60: Synthesis of N-[3-[1-(4-chloro-1H-imidazol-2-yl)imidazo[1,5-a]pyridin-6-yl]-2,4-difluorophenyl]-5-fluoro-2-methoxypyridine-3-sulfonamide (Compound 45)

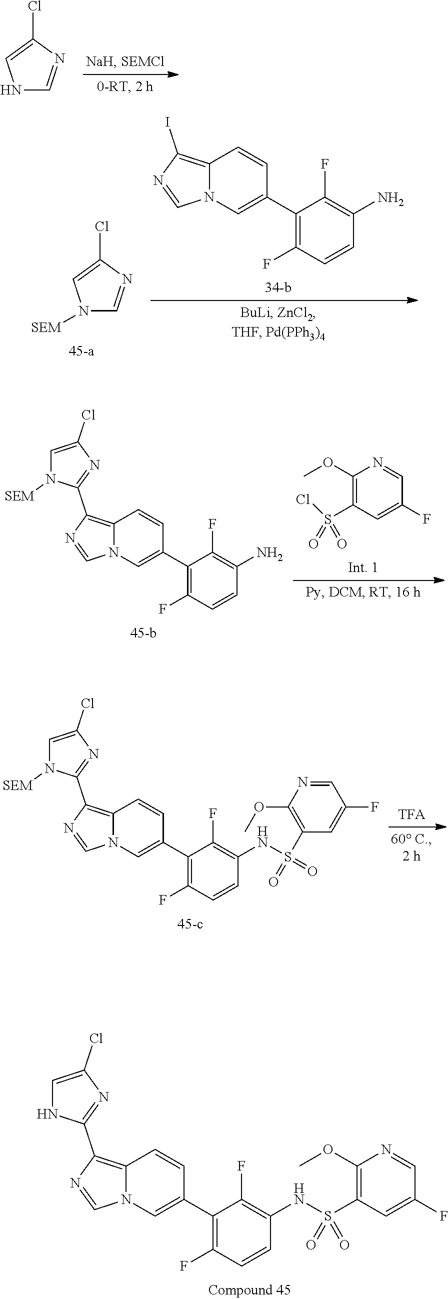

### Synthesis of 45-a: 1-[[2-(trimethylsilyl)ethoxy]methyl]imidazole-4-carbonitrile

Into a 100 mL 3-necked round-bottom flask were added 4-chloro-1H-imidazole (2 g, 20 mmol, 1 equiv) and DMF (20 mL). To the above mixture was added 60% NaH (1.4 g, 58.5 mmol, 3 equiv) in portions at 0° C. The resulting mixture was stirred for additional 30 min at 0° C., then[2-(chloromethoxy)ethyl]trimethylsilane (3.58 g, 21.4 mmol, 1.1 equiv) was added dropwise at 0° C. The resulting mixture was stirred for 1 h at room temperature, then quenched with water (100 mL). The resulting mixture was diluted with water (100 mL) and extracted with EtOAc (3×100 mL). The combined organics were washed with brine (3×50 mL), dried over anhydrous Na₂SO₄ and concentrated under reduced pressure. The residue was purified by silica gel column chromatography, eluting with PE/EtOAc (1:1) to afford 1-[[2-(trimethylsilyl)ethoxy]methyl]imidazole-4-carbonitrile (3.2 g, 67% yield) as a colorless oil.

LCMS (ES, m/z): [M−H]⁻: 233

### Synthesis of 45-b: 3-[I-(4-chloro-1-[[2-(trimethylsilyl)ethoxy]methyl]imidazol-2-yl)imidazo[1,5-a]pyridin-6-yl]-2,4-difluoroaniline

Into a 100 mL 3-necked round-bottom flask were added 4-chloro-1-[[2-(trimethylsilyl)ethoxy]methyl]imidazole (1.6 g, 6.8 mmol, 1 equiv) and THE (20 mL). To this was added 2.5M n-BuLi (3.3 mL, 8.2 mmol, 1.2 equiv) dropwise at −78° C. The resulting mixture was stirred for 1 h at −78° C., then 1M ZnCl₂ in Et₂O (8.1 mL, 8.1 mmol, 1.2 equiv) was added and this mixture stirred for 1 h, allowing it to warm to room temperature. 2,4-Difluoro-3-[1-iodoimidazo[1,5-a]pyridin-6-yl]aniline (1 g, 2.7 mmol, 0.39 equiv) and Pd(PPh₃)₄ (790 mg, 0.68 mmol, 0.1 equiv) were added, and this stirred for 1 h at 60° C. The resulting mixture was diluted with water (100 mL) and extracted with EtOAc (3×100 mL). The combined organics were washed with brine (3×50 mL), dried over anhydrous Na₂SO₄ and concentrated under reduced pressure. The residue was purified by silica gel column chromatography, eluting with PE/EtOAc (3:2) to afford 3-[1-(4-chloro-1-[[2-(trimethylsilyl)ethoxy]methyl]imidazol-2-yl)imidazo[1,5-a]pyridin-6-yl]-2,4-difluoroaniline (1.1 g, 34% yield) as a light brown solid.

LCMS (ES, m/z): [M+H]⁺: 476.

### Synthesis of 45-c: N-[3-[1-(4-chloro-1-[[2-(trimethylsilyl)ethoxy]methyl]imidazol-2-yl)imidazo[1,5-a]pyridin-6-yl]-2,4-difluorophenyl]-5-fluoro-2-methoxypyridine-3-sulfonamide

Into an 8 mL vial were added 3-[1-(4-chloro-1-[[2-(trimethylsilyl)ethoxy]methyl]imidazol-2-yl)imidazo[1,5-a]pyridin-6-yl]-2,4-difluoroaniline (150 mg, 0.3 mmol, 1 equiv), 5-fluoro-2-methoxypyridine-3-sulfonyl chloride (106 mg, 0.47 mmol, 1.5 equiv), DCM (2 mL) and pyridine (124 mg, 1.56 mmol, 5 equiv). The resulting mixture was stirred overnight at room temperature. The mixture was concentrated under vacuum and the residue purified by silica gel column chromatography, eluting with PE/EtOAc (1:1) to afford N-[3-[1-(4-chloro-1-[[2-(trimethylsilyl)ethoxy]methyl]imidazol-2-yl)imidazo[1,5-a]pyridin-6-yl]-2,4-difluorophenyl]-5-fluoro-2-methoxypyridine-3-sulfonamide (120 mg, 57% yield) as a light yellow solid.

LCMS (ES, m/z): [M+H]⁺: 665.

### Synthesis of Compound 45: N-[3-[1-(4-chloro-1H-imidazol-2-yl)imidazo[1,5-a]pyridin-6-yl]-2,4-difluorophenyl]-5-fluoro-2-methoxypyridine-3-sulfonamide

Into an 8 mL vial were added N-[3-[1-(4-chloro-1-[[2-(trimethylsilyl)ethoxy]methyl]imidazol-2-yl)imidazo[1,5-a]pyridin-6-yl]-2,4-difluorophenyl]-5-fluoro-2-methoxypyridine-3-sulfonamide (110 mg, 0.16 mmol, 1 equiv) and TFA (2 mL).

The resulting mixture was stirred for 2 h at 60° C., then concentrated under vacuum. The crude product was purified by Prep-HPLC with the following conditions: Column: Atlantis HILIC OBD, 19*150 mm*5 um; Mobile Phase 30-70% MeCN/0.1% aqueous formic acid; Flow rate: 90 mL/min; to afford N-[3-[1-(4-chloro-1H-imidazol-2-yl)imidazo[1,5-a]pyridin-6-yl]-2,4-difluorophenyl]-5-fluoro-2-methoxypyridine-3-sulfonamide (44 mg, 50% yield) as a light green solid.

LCMS (ES, m/z): [M+H]⁺: 535.

¹H NMR (300 MHz, DMSO-d₆) δ 12.73 (s, 1H), 10.44 (s, 1H), 8.54 (s, 2H), 8.42 (d, J=3.0 Hz, 1H), 8.14 (d, J=9.5 Hz, 1H), 8.00 (dd, J=7.4, 3.0 Hz, 1H), 7.41-7.27 (m, 1H), 7.18 (d, J=2.2 Hz, 2H), 6.90 (d, J=9.4 Hz, 1H), 3.90 (s, 3H).

### Example 61: Synthesis of N-[2,4-difluoro-3-[1-(1-methylpyrazol-3-yl)imidazo[1,5-a]pyridin-6-yl]phenyl]-5-fluoro-2-methoxypyridine-3-sulfonamide (Compound 46)

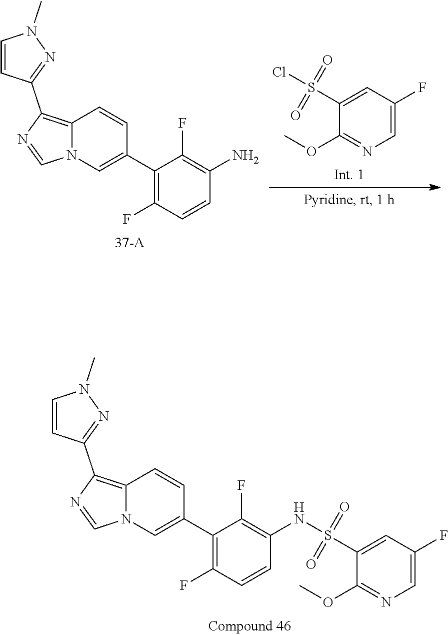 [SMILES: Cn1ccc(-c2ncn3cc(-c4c(F)ccc(N)c4F)ccc23)n1 ; COc1ncc(F)cc1S(=O)(=O)Cl ; COc1ncc(F)cc1S(=O)(=O)Nc1ccc(F)c(-c2ccc3c(-c4ccn(C)n4)ncn3c2)c1F]

### Synthesis of Compound 46: N-[2,4-difluoro-3-[1-(1-methylpyrazol-3-yl)imidazo[1,5-a]pyridin-6-yl]phenyl]-5-fluoro-2-methoxypyridine-3-sulfonamide

Into an 8 mL vial was placed 2,4-difluoro-3-[1-(1-methylpyrazol-3-yl)imidazo[1,5-a]pyridin-6-yl]aniline (80 mg, 0.25 mmol, 1 equiv), pyridine (2 mL) and 5-fluoro-2-methoxypyridine-3-sulfonyl chloride (83 mg, 0.4 mmol, 1.5 equiv) at room temperature. The resulting solution was stirred for 1 h at room temperature. The resulting mixture was concentrated under vacuum and the residue purified by prep-HPLC with the following conditions: Column, welch Vltimate XB-C18, 50×250 mm, 10 μm; Mobile Phase 20-55% MeCN/0.1% aqueous formic acid; to afford N-[2,4-difluoro-3-[1-(1-methylpyrazol-3-yl)imidazo[1,5-a]pyridin-6-yl]phenyl]-5-fluoro-2-methoxypyridine-3-sulfonamide (50 mg, 40% yield) as a light yellow solid.

LCMS (ES, m/z): [M+H]⁺: 515

¹H NMR (300 MHz, DMSO-d₆) δ 10.42 (s, 1H), 8.46 (d, J=6.6 Hz, 3H), 8.10 (d, J=9.4 Hz, 1H), 8.03 (dd, J=7.4, 3.0 Hz, 1H), 7.73 (d, J=2.3 Hz, 1H), 7.36 (d, J=6.1 Hz, 1H), 7.24 (d, J=9.2 Hz, 1H), 6.80-6.71 (m, 1H), 6.62 (d, J=2.2 Hz, 1H), 3.92 (s, 6H).

### Example 62: Synthesis of N-[2,4-difluoro-3-[8-fluoro-3-(1H-imidazol-2-yl)imidazo[1,5-a]pyridin-7-yl]phenyl]-5-fluoro-2-methoxypyridine-3-sulfonamide (Compound 47)

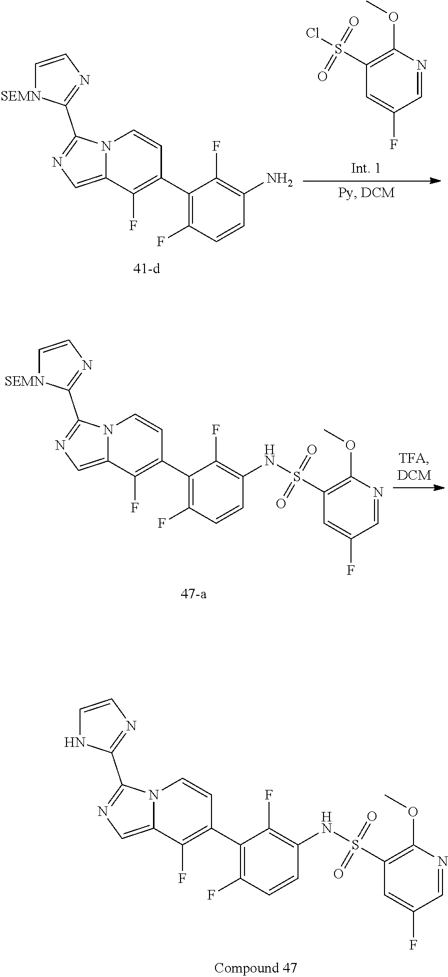 [SMILES: C[Si](C)(C)CCOCn1ccnc1-c1ncc2c(F)c(-c3c(F)ccc(N)c3F)ccn12 ; COc1ncc(F)cc1S(=O)(=O)Cl ; COc1ncc(F)cc1S(=O)(=O)Nc1ccc(F)c(-c2ccn3c(-c4nccn4COCC[Si](C)(C)C)ncc3c2F)c1F ; COc1ncc(F)cc1S(=O)(=O)Nc1ccc(F)c(-c2ccn3c(-c4ncc[nH]4)ncc3c2F)c1F]

### Synthesis of 47-a: N-[2,4-difluoro-3-[8-fluoro-3-(1-[[2-(trimethylsilyl)ethoxy]methyl]imidazol-2-yl)imidazo[1,5-a]pyridin-7-yl]phenyl]-5-fluoro-2-methoxypyridine-3-sulfonamide

To a solution of 2,4-difluoro-3-[8-fluoro-3-(1-[[2-(trimethylsilyl)ethoxy]methyl]imidazol-2-yl)imidazo[1,5-a]pyridin-7-yl]aniline (150 mg, 0.3 mmol, 1 equiv) and pyridine (77 mg, 0.9 mmol, 3 equiv) in DCM (5 mL) was added 5-fluoro-2-methoxypyridine-3-sulfonyl chloride (88 mg, 0.39 mmol, 1.2 equiv) in portions at room temperature. The resulting solution was stirred for 1 h, then concentrated. The residue was purified by column chromatography over silica gel (eluent: PE:EA=4:1) to afford N-[2,4-difluoro-3-[8-fluoro-3-(1-[[2-(trimethylsilyl)ethoxy]methyl]imidazol-2-yl)imidazo[1,5-a]pyridin-7-yl]phenyl]-5-fluoro-2-methoxypyridine-3-sulfonamide (100 mg, 47% yield) as a yellow solid.

LCMS (ES, m/z): [M+H]⁺: 649

### Synthesis of Compound 47: N-[2,4-difluoro-3-[8-fluoro-3-(1H-imidazol-2-yl)imidazo[1,5-a]pyridin-7-yl]phenyl]-5-fluoro-2-methoxypyridine-3-sulfonamide

To a stirred solution of N-[2,4-difluoro-3-[8-fluoro-3-(1-[[2-(trimethylsilyl)ethoxy]methyl]imidazol-2-yl)imidazo[1,5-a]pyridin-7-yl]phenyl]-5-fluoro-2-methoxypyridine-3-sulfonamide (70 mg, 0.1 mmol, 1 equiv) in DCM (4 mL) was added TFA (1 mL) dropwise at room temperature. The resulting mixture was stirred for 1 h then concentrated under reduced pressure. The residue was purified by prep-HPLC with following conditions: Column, welch Vltimate XB-C18, 50×250 mm, 10 m; Mobile Phase: 30-65% MeCN/0.1% aqueous formic acid; Detector, 220 nm; to afford N-[2,4-difluoro-3-[8-fluoro-3-(1H-imidazol-2-yl)imidazo[1,5-a]pyridin-7-yl]phenyl]-5-fluoro-2-methoxypyridine-3-sulfonamide (30 mg, 53% yield) as a white solid.

LCMS (ES, m/z): [M+H]⁺: 519

¹H NMR (300 MHz, DMSO-d₆) δ 13.11 (s, 1H), 10.46 (s, 1H), 9.44 (dd, J=7.3, 0.9 Hz, 1H), 8.45 (d, J=3.0 Hz, 1H), 8.01 (dd, J=7.4, 3.0 Hz, 1H), 7.86 (d, J=0.9 Hz, 1H), 7.44 (td, J=8.9, 5.9 Hz, 1H), 7.36-7.13 (m, 3H), 6.85 (t, J=6.9 Hz, 1H), 3.90 (s, 3H).

### Example 63: Synthesis of 5-chloro-N-[2,4-difluoro-3-[1-(4-methyl-3H-imidazol-2-yl)imidazo[1,5-a]pyridine-6-yl]phenyl]-2-methoxypyridine-3-sulfonamide (Compound

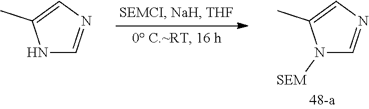

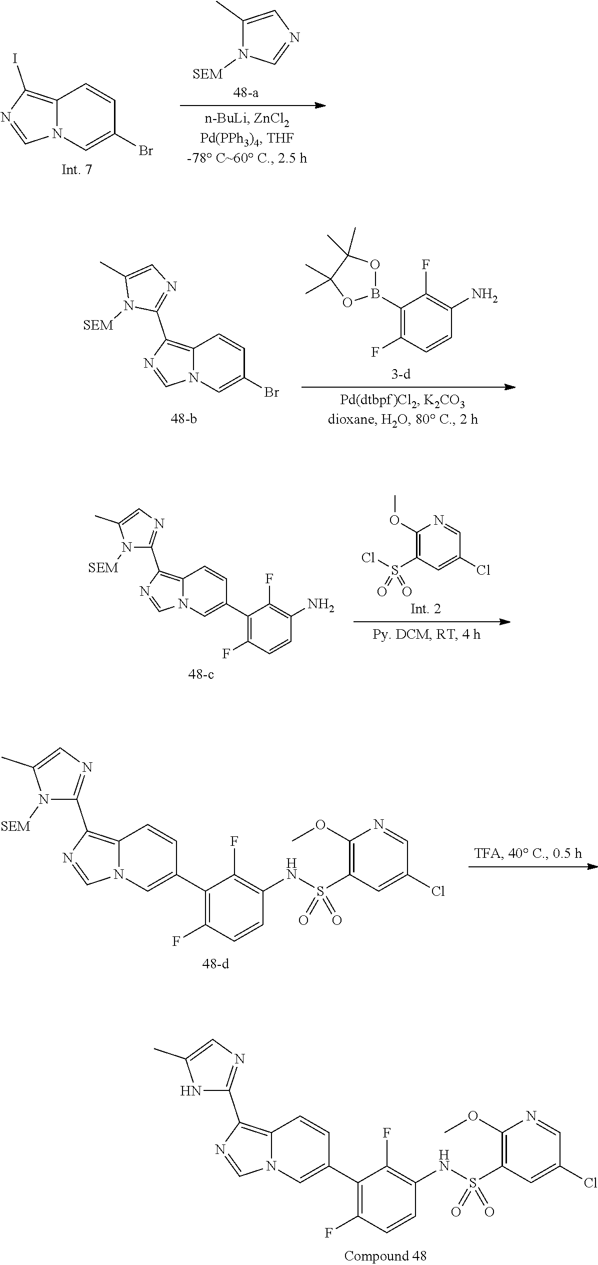

### Synthesis of 48-a: 5-methyl-1-[[2-(trimethylsilyl)ethoxy]methyl]imidazole

To a solution of 4-methylimidazole (5 g, 61 mmol, 1 equiv) in DMF (50 mL) was added NaH (4.9 g, 122 mmol, 2 equiv, 60% in oil) at 0° C. The mixture was stirred for 15 min, then SEMCI (12.2 g, 73 mmol, 1.2 equiv) was added and the mixture was allowed to warm to room temperature over 16 h. The reaction mixture was quenched by water (25 mL) and extracted with DCM (3×25 mL). The combined organics were washed with water and brine (50 mL), dried over anhydrous Na₂SO₄ and concentrated under reduced pressure. The residue was purified by silica gel column chromatography, eluting with PE/EA (10/1) to afford 5-methyl-1-[[2-(trimethylsilyl)ethoxy]methyl]imidazole (8 g, 62% yield) as a light yellow oil.

LCMS (ES, m/z): [M+H]⁺: 213.

### Synthesis of 48-b: 2-[6-bromoimidazo[1,5-a]pyridin-1-yl]-5-methyl-1-[[2-(trimethylsilyl)ethoxy]methyl]imidazole

In a 50 mL round bottom flask, to a solution of 5-methyl-1-[[2-(trimethylsilyl)ethoxy]methyl]imidazole (197 mg, 0.93 mmol, 3 equiv) in THE (2 mL) was added dropwise n-butyllithium solution (2.5 M in hexane, 0.37 mL, 0.93 mmol, 3 equiv) at −78° C. under N₂ atmosphere. The reaction mixture was stirred at −78° C. for 30 mins, then ZnCl₂ (1 M in Et₂O, 1 mL, 1 mmol, 3.3 equiv) was added dropwise at −78° C. The resulting mixture was stirred for 30 mins at room temperature, then a solution of 6-bromo-1-iodoimidazo[1,5-a]pyridine (100 mg, 0.3 mmol, 1 equiv) and Pd(PPh₃)₄ (71 mg, 0.062 mmol, 0.2 equiv) in 0.5 mL THE was added dropwise. The resulting mixture was stirred for 60 mins at 60° C., then quenched with sat. NH₄Cl solution (2 mL). The mixture was extracted with EtOAc (2×30 mL) and the combined organics were washed with brine (10 mL), dried over anhydrous Na₂SO₄, and concentrated under vacuum. The residue was purified by silica gel column chromatography, eluting with PE/EA (3/1) to afford 2-[6-bromoimidazo[1,5-a]pyridin-1-yl]-5-methyl-1-[[2-(trimethylsilyl)ethoxy]methyl]imidazole (86 mg, 45% yield) as a light brown solid.

LCMS (ES, m/z): [M+H]⁺: 407.

### Synthesis of 48-c: 2,4-difluoro-3-[1-(5-methyl-1-[[2-(trimethylsilyl)ethoxy]methyl]imidazol-2-yl)imidazo[1,5-a]pyridin-6-yl]aniline

To a solution of 2-[6-bromoimidazo[1,5-a]pyridin-1-yl]-5-methyl-1-[[2-(trimethylsilyl)ethoxy]methyl]imidazole (76 mg, 0.19 mmol, 1 equiv) and 2,4-difluoro-3-(4,4,5,5-tetramethyl-1,3,2-dioxaborolan-2-yl)aniline (72 mg, 0.28 mmol, 1.5 equiv) in dioxane (2 mL) and H₂O (0.2 mL) were added K₂CO₃ (77 mg, 0.56 mmol, 3 equiv) and Pd(dppf)Cl₂ (14 mg, 0.019 mmol, 0.1 equiv). After stirring for 2 h at 80° C. under a nitrogen atmosphere, the resulting mixture was concentrated under reduced pressure. The residue was purified by Prep-TLC/silica gel column chromatography, eluting with PE/EA (1/1) to afford 2,4-difluoro-3-[1-(5-methyl-1-[[2-(trimethylsilyl)ethoxy]methyl]imidazol-2-yl)imidazo[1,5-a]pyridin-6-yl]aniline (53 mg, 62% yield) as a light brown solid.

LCMS (ES, m/z): [M+H]⁺: 456.

### Synthesis of 48-d: 5-chloro-N-[2,4-difluoro-3-[1-(5-methyl-1-[[2-(trimethylsilyl)ethoxy]methyl]imidazol-2-yl)imidazo[1,5-a]pyridin-6-yl]phenyl]-2-methoxypyridine-3-sulfonamide

To a stirred solution of 2,4-difluoro-3-[1-(5-methyl-1-[[2-(trimethylsilyl)ethoxy]methyl]imidazol-2-yl)imidazo[1,5-a]pyridin-6-yl]aniline (43 mg, 0.09 mmol, 1 equiv) and pyridine (30 mg, 0.38 mmol, 4 equiv) in DCM (2 mL) was added 5-chloro-2-methoxypyridine-3-sulfonyl chloride (46 mg, 0.19 mmol, 2 equiv) in portions at room temperature. The resulting mixture was stirred 4 h, then diluted with water (2 mL). This was extracted with CH₂Cl₂ (3×10 mL), and the combined organics were washed with water and brine, dried over anhydrous Na₂SO₄ and concentrated under reduced pressure. The residue was purified by silica gel column chromatography, eluting with EtOAc/PE (0-50%) to afford 5-chloro-N-[2,4-difluoro-3-[1-(5-methyl-1-[[2-(trimethylsilyl)ethoxy]methyl]imidazol-2-yl)imidazo[1,5-a]pyridin-6-yl]phenyl]-2-methoxypyridine-3-sulfonamide (52 mg, 84% yield) as a yellow solid.

LCMS (ES, m/z): [M+H]⁺: 661.

### Synthesis of Compound 48: 5-chloro-N-[2,4-difluoro-3-[1-(4-methyl-3H-imidazol-2-yl)imidazo[1,5-a]pyridine-6-yl]phenyl]-2-methoxypyridine-3-sulfonamide

To a stirred mixture of 5-chloro-N-[2,4-difluoro-3-[1-(5-methyl-1-[[2-(trimethylsilyl)ethoxy]methyl]imidazol-2-yl)imidazo[1,5-a]pyridin-6-yl]phenyl]-2-methoxypyridine-3-sulfonamide (55 mg, 0.08 mmol, 1 equiv) was added DCM (1 mL) and TFA (0.5 mL) at room temperature. The resulting mixture was stirred for 30 min at 40° C.

The resulting mixture was concentrated under vacuum and the residue neutralized to pH 7 with ammonia. This was purified by Prep-HPLC: Column: Sunfire Prep C₁₈ OBD, 50×250 mm, 5 um-10 nm; Mobile Phase: 18-45% 1:1 MeOH:MeCN/0.1% aqueous ammonia; Flow rate: 90 mL/min; to afford 5-chloro-N-[2,4-difluoro-3-[1-(4-methyl-3H-imidazol-2-yl)imidazo[1,5-a]pyridin-6-yl]phenyl]-2-methoxypyridine-3-sulfonamide (17 mg, 39% yield) as a light yellow solid.

LCMS (ES, m/z): [M+H]⁺: 531.

¹H NMR (300 MHz, DMSO-d6): δ 8.50 (m, 3H), 8.20 (d, J=9.4 Hz, 1H), 8.08 (d, J=2.6 Hz, 1H), 7.36 (td, J=8.9, 5.9 Hz, 1H), 7.29-7.17 (m, 1H), 6.86-6.73 (m, 2H), 3.92 (s, 3H), 2.21 (s, 3H).

### Example 64: Synthesis of 5-chloro-N-(2,4-difluoro-3-[1-[4-(hydroxymethyl)-3H-imidazol-2-yl]imidazo[1,5-a]pyridin-6-yl]phenyl)-2-methoxypyridine-3-sulfonamide (Compound 49)

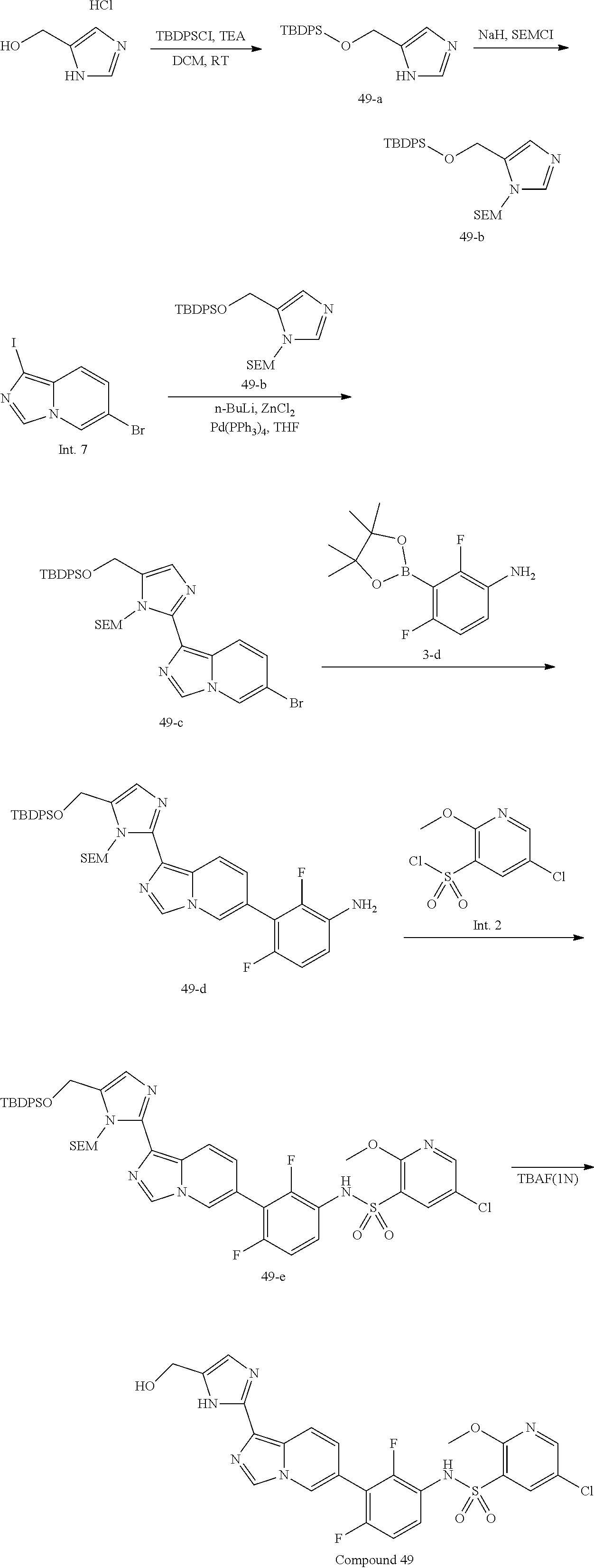

### Synthesis of 49-a: 4-[[(tert-butyldiphenylsilyl)oxy]methyl]-1H-imidazole

To a stirred mixture of 3H-imidazol-4-ylmethanol hydrochloride (5 g, 37 mmol, 1 equiv) and Et₃N (7.5 g, 74 mmol, 2 equiv) in DCM (50 mL) was added tert-butyl(chloro)diphenylsilane (15.3 g, 55 mmol, 1.5 equiv) dropwise at 0-5° C. The resulting mixture was stirred overnight at room temperature, then diluted with water (100 mL). The resulting mixture was extracted with DCM (3×100 mL) and the combined organics were washed with water and brine, dried over anhydrous Na₂SO₄, then concentrated under reduced pressure. The residue was purified by silica gel column chromatography, eluting with 0-50% EtOAc/PE to afford 4-[[(tert-butyldiphenylsilyl)oxy]methyl]-1H-imidazole (6.4 g, 51% yield) as a white solid.

### Synthesis of 49-b: 5-[[(tert-butyldiphenylsilyl)oxy]methyl]-1-[[2-(trimethylsilyl)ethoxy]methyl]imidazole

To a stirred solution of 4-[[(tert-butyldiphenylsilyl)oxy]methyl]-3H-imidazole (6.5 g, 19.3 mmol, 1 equiv) in DMF (65 mL) was added NaH (1.55 g, 38.6 mmol, 2 equiv, 60% in oil) in portions at 0-5° C. The resulting mixture was stirred for 10 min then SEM-Cl (4.83 g, 28.9 mmol, 1.5 equiv) was added dropwise at 0-5° C. The resulting mixture was stirred for 2 h at room temperature, then diluted with water (200 mL). This was extracted with EtOAc (3×200 mL) and the combined organics were washed with water and brine, dried over anhydrous Na₂SO₄, then concentrated under reduced pressure. The residue was purified by silica gel column chromatography, eluting with PE/EtOAc (3:1) to afford 5-[[(tert-butyldiphenylsilyl)oxy]methyl]-1-[[2-(trimethylsilyl)ethoxy]methyl]imidazole (4 g, 44% yield) as a yellow oil.

LCMS (ES, m/z): [M+H]: 467.

### Synthesis of 49-c: 2-[6-bromoimidazo[1,5-a]pyridin-1-yl]-5-[[(tert-butyldiphenylsilyl)oxy]methyl]-1-[[2-(trimethylsilyl)ethoxy]methyl]imidazole

To a stirred solution of 5-[[(tert-butyldiphenylsilyl)oxy]methyl]-1-[[2-(trimethylsilyl)ethoxy]methyl]imidazole (4.34 g, 9.3 mmol, 3 equiv) in THE (40 mL) was added n-BuLi (595 mg, 9.3 mmol, 3 equiv) at −78° C. under nitrogen atmosphere and this was stirred for 30 min at −78° C. ZnCl₂ (1.27 g, 9.3 mmol, 3 equiv) was added at −78° C., then the solution was warmed to room temperature and stirred for additional 30 min. Pd(PPh₃)₄ (715 mg, 0.62 mmol, 0.2 equiv) and 6-bromo-1-iodoimidazo[1,5-a]pyridine (1 g, 3.1 mmol, 1 equiv) were added and the resulting mixture was stirred for 1 h at 50° C. The mixture was allowed to cool to room temperature and was quenched with water (100 mL). The resulting mixture was extracted with EtOAc (2×50 mL), and the combined organics were washed with brine (50 mL), dried over anhydrous Na₂SO₄, then concentrated under reduced pressure.

The residue was purified by silica gel column chromatography, eluting with PE/THF (80:20) to afford 2-[6-bromoimidazo[1,5-a]pyridin-1-yl]-5-[[(tert-butyldiphenylsilyl)oxy]methyl]-1-[[2-(trimethylsilyl) ethoxy]methyl]imidazole (850 mg, 41% yield) as a red oil.

LCMS (ES, m/z): [M+H]: 661, 663

### Synthesis of 49-d: 3-[i-(5-[[(tert-butyldiphenylsilyl)oxy]methyl]-1-[[2-(trimethylsilyl)ethoxy]methyl]imidazol-2-yl)imidazo[1,5-a]pyridin-6-yl]-2,4-difluoroaniline

Into a 40 mL vial were added 2-[6-bromoimidazo[1,5-a]pyridin-1-yl]-5-[[(tert-butyldiphenylsilyl)oxy]methyl]-1-[[2-(trimethylsilyl)ethoxy]methyl]imidazole (870 mg, 1.3 mmol, 1 equiv), dioxane (9 mL) and H₂O (1 mL). To the stirred solution was added 2,4-difluoro-3-(4,4,5,5-tetramethyl-1,3,2-dioxaborolan-2-yl)aniline (503 mg, 1.9 mmol, 1.5 equiv), K₂CO₃ (545 mg, 3.9 mmol, 3 equiv) and Pd(dtbpf)Cl₂ (85 mg, 0.13 mmol, 0.1 equiv) at room temperature under nitrogen atmosphere. The resulting mixture was stirred for 1 h at 80° C. under nitrogen atmosphere, then cooled to room temperature. 2,4-Difluoro-3-(4,4,5,5-tetramethyl-1,3,2-dioxaborolan-2-yl)aniline (503 mg, 1.972 mmol, 1.5 equiv) and Pd(dtbpf)Cl₂ (85 mg, 0.13 mmol, 0.1 equiv) were added and the mixture was stirred for an additional 1 h at 80° C. The reaction was allowed to cool and quenched with water (40 mL).

The resulting mixture was extracted with EtOAc (3×30 mL), and the combined organics washed with brine (20 mL), dried over anhydrous Na₂SO₄, then concentrated under reduced pressure. The residue was purified by silica gel column chromatography, eluting with PE/THF (1:1) to afford 3-[1-(5-[[(tert-butyldiphenylsilyl)oxy]methyl]-1-[[2-(trimethylsilyl)ethoxy]methyl]imidazol-2-yl)imidazo[1,5-a]pyridin-6-yl]-2,4-difluoroaniline (600 mg, 64% yield) as a brown oil.

LCMS (ES, m/z): [M+H]⁺: 710.

### Synthesis of 49-e: N-[3-[1-(5-[[(tert-butyldiphenylsilyl)oxy]methyl]-1-[[2-(trimethylsilyl)ethoxy]methyl]imidazol-2-yl)imidazo[1,5-a]pyridin-6-yl]-2,4-difluorophenyl]-5-chloro-2-methoxypyridine-3-sulfonamide

Into a 4 mL vial were added 3-[1-(5-[[(tert-butyldiphenylsilyl)oxy]methyl]-1-[[2-(trimethylsilyl)ethoxy]methyl]imidazol-2-yl)imidazo[1,5-a]pyridin-6-yl]-2,4-difluoroaniline (120 mg, 0.17 mmol, 1 equiv) and DCM (4 mL). To the stirred solution was added pyridine (40 mg, 0.5 mmol, 3 equiv) and 5-chloro-2-methoxypyridine-3-sulfonyl chloride (61 mg, 0.25 mmol, 1.5 equiv) and the resulting mixture was stirred overnight at room temperature. The reaction was quenched with water (20 mL), and extracted with EtOAc (3×20 mL). The combined organics were washed with brine (20 mL), dried over anhydrous Na₂SO₄ and concentrated under reduced pressure. The residue was purified by silica gel column chromatography, eluting with PE/THF (40:60) to afford N-[3-[1-(5-[[(tert-butyldiphenylsilyl)oxy]methyl]-1-[[2-(trimethylsilyl)ethoxy]methyl]imidazole-2-yl)imidazo[1,5-a]pyridin-6-yl]-2,4-difluorophenyl]-5-chloro-2-methoxypyridine-3-sulfonamide (120 mg, 78% yield) as a brown oil.

LCMS (ES, m/z): [M+H]⁺: 915.

### Synthesis of Compound 49: 5-chloro-N-(2,4-difluoro-3-[1-[4-(hydroxymethyl)-3H-imidazol-2-yl]imidazo[1,5-a]pyridin-6-yl]phenyl)-2-methoxypyridine-3-sulfonamide

Into a 20 mL vial were added N-[3-[1-(5-[[(tert-butyldiphenylsilyl)oxy]methyl]-1-[[2-(trimethylsilyl)ethoxy]methyl]imidazol-2-yl)imidazo[1,5-a]pyridin-6-yl]-2,4-difluorophenyl]-5-chloro-2-methoxypyridine-3-sulfonamide (140 mg, 0.15 mmol, 1 equiv) and 1 M TBAF in THE (4 mL). The resulting mixture was stirred overnight at 80° C., then concentrated. The crude product was purified by Prep-HPLC with the following conditions: Column: welch Vltimate XB-C18, 50×250 mm, 10 μm; Flowrate: 90 mL/min; Mobile Phase: 5-40% MeCN/0.05% aqueous formic acid; to afford 5-chloro-N-(2,4-difluoro-3-[1-[4-(hydroxymethyl)-3H-imidazol-2-yl]imidazo[1,5-a]pyridin-6-yl]phenyl)-2-methoxypyridine-3-sulfonamide (25 mg, 30% yield) as a light yellow solid.

LCMS (ES, m/z): [M+H]⁺: 547

¹H NMR (300 MHz, DMSO-d₆) δ 8.52 (m, 3H), 8.23 (d, J=9.4 Hz, 1H), 8.11-8.05 (m, 1H), 7.37 (td, J=8.8, 5.9 Hz, 1H), 7.25 (td, J=9.2, 1.6 Hz, 1H), 6.93 (s, 1H), 6.83 (dd, J=9.4, 1.6 Hz, 1H), 4.84 (s, 1H), 4.45 (d, J=3.7 Hz, 2H), 3.93 (s, 3H).

### Example 65: Synthesis of N-[2,4-difluoro-3-[1-(4-methyl-3H-imidazol-2-yl)imidazo[1,5-a]pyridin-6-yl]phenyl]-5-fluoro-2-methylpyridine-3-sulfonamide (Compound 50)

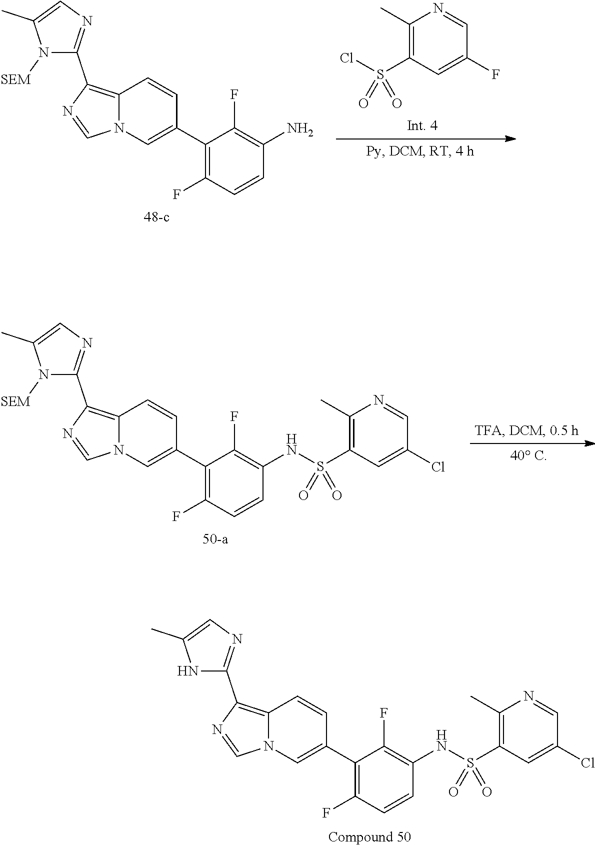 [SMILES: Cc1cnc(-c2ncn3cc(-c4c(F)ccc(N)c4F)ccc23)n1COCC[Si](C)(C)C ; Cc1ncc(F)cc1S(=O)(=O)Cl ; Cc1ncc(Cl)cc1S(=O)(=O)Nc1ccc(F)c(-c2ccc3c(-c4ncc(C)n4COCC[Si](C)(C)C)ncn3c2)c1F ; Cc1cnc(-c2ncn3cc(-c4c(F)ccc(NS(=O)(=O)c5cc(Cl)cnc5C)c4F)ccc23)[nH]1]

### Synthesis of 50-a: N-[2,4-difluoro-3-[1-(5-methyl-1-[[2-(trimethylsilyl)ethoxy]methyl]imidazol-2-yl)imidazo[1,5-a]pyridin-6-yl]phenyl]-5-fluoro-2-methylpyridine-3-sulfonamide

To a stirred solution of 2,4-difluoro-3-[1-(5-methyl-1-[[2-(trimethylsilyl)ethoxy]methyl]imidazol-2-yl)imidazo[1,5-a]pyridin-6-yl]aniline (200 mg, 0.44 mmol, 1 equiv) and pyridine (139 mg, 1.76 mmol, 4 equiv) in DCM (2 mL) was added 5-fluoro-2-methylpyridine-3-sulfonyl chloride (184 mg, 0.88 mmol, 2 equiv) in portions at room temperature. The resulting mixture was stirred for 4 h at room temperature, then diluted with water (2 mL) and extracted with CH₂Cl₂ (3×10 mL). The combined organics were washed with water and brine, dried over anhydrous Na₂SO₄ and concentrated under reduced pressure. The residue was purified by silica gel column chromatography, eluting with EtOAc/PE (0-50%) to afford N-[2,4-difluoro-3-[1-(5-methyl-1-[[2-(trimethylsilyl)ethoxy]methyl]imidazol-2-yl)imidazo[1,5-a]pyridin-6-yl]phenyl]-5-fluoro-2-methylpyridine-3-sulfonamide (163 mg, 59% yield) as a yellow solid.

LCMS (ES, m/z): [M+H]⁺: 629.

### Synthesis of Compound 50: N-[2,4-difluoro-3-[1-(4-methyl-3H-imidazol-2-yl)imidazo[1,5-a]pyridin-6-yl]phenyl]-5-fluoro-2-methylpyridine-3-sulfonamide

To a stirred mixture of N-[2,4-difluoro-3-[1-(5-methyl-1-[[2-(trimethylsilyl)ethoxy]methyl]imidazol-2-yl)imidazo[1,5-a]pyridin-6-yl]phenyl]-5-fluoro-2-methylpyridine-3-sulfonamide (130 mg, 0.21 mmol, 1 equiv) was added DCM (1 mL) and TFA (0.5 mL) at room temperature. The resulting mixture was stirred for 30 min at 40° C., then concentrated under vacuum. The residue was neutralized to pH 7 with 5% aqueous NH₃, and was purified by Prep-HPLC: Column, Sunfire Prep C18 OBD, 50*250 mm, 5 um-10 nm; Mobile Phase 9-26% 1:1 MeOH:MeCN/0.1% aqueous ammonia; Flow rate: 90 mL/min; to afford N-[2,4-difluoro-3-[1-(4-methyl-3H-imidazol-2-yl)imidazo[1,5-a]pyridin-6-yl]phenyl]-5-fluoro-2-methylpyridine-3-sulfonamide (10 mg, 10% yield) as a light yellow solid.

LCMS (ES, m/z): [M+H]⁺: 499.

¹H NMR (300 MHz, DMSO-d₆): δ 11.95-11.68 (br, 1H), 8.70 (d, J=2.8 Hz, 1H), 8.51 (d, J=4.8 Hz, 2H), 8.20 (d, J=9.5 Hz, 1H), 7.95 (dd, J=8.3, 2.8 Hz, 1H), 7.33 (td, J=8.8, 5.6 Hz, 1H), 7.20 (t, J=9.2 Hz, 1H), 6.84 (s, 2H), 2.78 (s, 3H), 2.23 (s, 3H).

### Example 66: Synthesis of N-(2,4-difluoro-3-[1-[4-(hydroxymethyl)-3H-imidazol-2-yl]imidazo[1,5-a]pyridin-6-yl]phenyl)-5-fluoro-2-methylpyridine-3-sulfonamide (Compound 51)

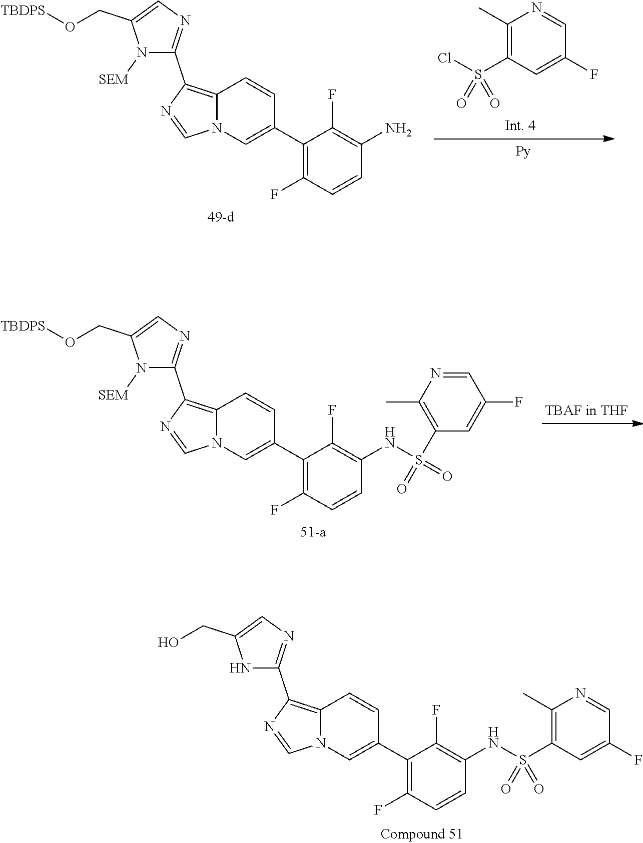

### Synthesis of 51-a: N-[3-[1-(5-[[(tert-butyldiphenylsilyloxy)methyl]-1-[[2-(trimethylsilyl)ethoxy]methyl]imidazol-2-yl)imidazo[1,5-a]pyridin-6-yl]-2,4-difluorophenyl]-5-fluoro-2-methylpyridine-3-sulfonamide

Into a 20 mL vial were added 3-[1-(5-[[(tert-butyldiphenylsilyl)oxy]methyl]-1-[[2-(trimethylsilyl)ethoxy]methyl]imidazol-2-yl)imidazo[1,5-a]pyridin-6-yl]-2,4-difluoroaniline (150 mg, 0.21 mmol, 1 equiv) and DCM (5 mL), followed by pyridine (83 mg, 1.06 mmol, 5 equiv) and 5-fluoro-2-methylpyridine-3-sulfonyl chloride (133 mg, 0.63 mmol, 3 equiv). The resulting mixture was stirred overnight at room temperature, then concentrated. The residue was purified by silica gel column chromatography, eluting with PE/THF (1:1) to afford N-[3-[1-(5-[[(tert-butyldiphenylsilyl)oxy]methyl]-1-[[2-(trimethylsilyl)ethoxy]methyl]imidazol-2-yl)imidazo[1,5-a]pyridin-6-yl]-2,4-difluorophenyl]-5-fluoro-2-methylpyridine-3-sulfonamide (100 mg, 54% yield) as a brown oil.

LCMS (ES, m/z): [M+H]⁺: 883.

### Synthesis of Compound 51: N-(2,4-difluoro-3-[1-[4-(hydroxymethyl)-3H-imidazol-2-yl]imidazo[1,5-a]pyridin-6-yl]phenyl)-5-fluoro-2-methylpyridine-3-sulfonamide

Into a 20 mL vial were added N-[3-[1-(5-[[(tert-butyldiphenylsilyl)oxy]methyl]-1-[[2-(trimethylsilyl)ethoxy]methyl]imidazol-2-yl)imidazo[1,5-a]pyridin-6-yl]-2,4-difluorophenyl]-5-fluoro-2-methylpyridine-3-sulfonamide (140 mg, 0.16 mmol, 1 equiv) and TBAF in THE (4 mL, 1M) at room temperature. The resulting mixture was stirred overnight at 70° C. under nitrogen atmosphere. The resulting mixture was diluted with sat. NH₄Cl (aq.) (10 mL) and extracted with EtOAc (3×10 mL). The combined organics were washed with brine (5 mL), dried over anhydrous Na₂SO₄ and concentrated under reduced pressure. The crude product was purified by Prep-HPLC with the following conditions: Column, welch Vltimate XB-C18, 50×250 mm, 10 μm; Flow rate 90 mL/min; Mobile phase: 5-40% MeCN/0.05% aqueous formic acid; to afford N-(2,4-difluoro-3-[1-[4-(hydroxymethyl)-3H-imidazol-2-yl]imidazo[1,5-a]pyridin-6-yl]phenyl)-5-fluoro-2-methylpyridine-3-sulfonamide (15 mg, 18% yield) as a light yellow solid.

LCMS (ES, m/z): [M+H]⁺: 515.

¹H NMR (300 MHz, DMSO-d₆) δ 8.71 (d, J=2.8 Hz, 1H), 8.51 (m, 2H), 8.21 (d, J=9.4 Hz, 1H), 7.95 (dd, J=8.2, 2.8 Hz, 1H), 7.34 (td, J=8.9, 5.9 Hz, 1H), 7.21 (t, J=9.3 Hz, 1H), 6.95 (s, 1H), 6.82 (d, J=9.4 Hz, 1H), 4.86 (s, 1H), 4.45 (s, 2H), 2.78 (d, J=1.2 Hz, 3H).

### Example 67: Synthesis of 5-chloro-N-[3-[1-(4-chloro-1H-imidazol-2-yl)imidazo[1,5-a]pyridin-6-yl]-2,4-difluorophenyl]-2-methoxypyridine-3-sulfonamide (Compound 52)

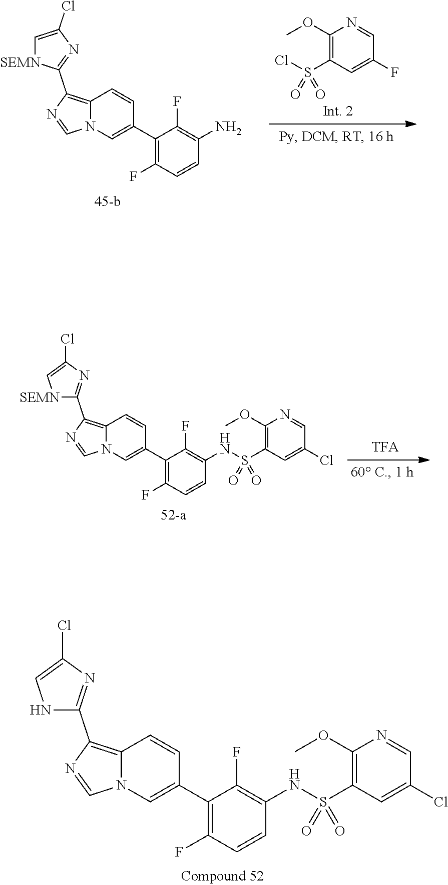 [SMILES: C[Si](C)(C)CCOCn1cc(Cl)nc1-c1ncn2cc(-c3c(F)ccc(N)c3F)ccc12 ; COc1ncc(F)cc1S(=O)(=O)Cl ; COc1ncc(Cl)cc1S(=O)(=O)Nc1ccc(F)c(-c2ccc3c(-c4nc(Cl)cn4COCC[Si](C)(C)C)ncn3c2)c1F ; COc1ncc(Cl)cc1S(=O)(=O)Nc1ccc(F)c(-c2ccc3c(-c4nc(Cl)c[nH]4)ncn3c2)c1F]

### Synthesis of 52-a: 5-chloro-N-[3-[1-(4-chloro-1-[[2-(trimethylsilyl)ethoxy]methyl]imidazol-2-yl) imidazo[1,5-a]pyridin-6-yl]-2,4-difluorophenyl]-2-methoxypyridine-3-sulfonamide

Into an 8 mL vial were added 3-[1-(4-chloro-1-[[2-(trimethylsilyl)ethoxy]methyl]imidazol-2-yl) imidazo[1,5-a]pyridin-6-yl]-2,4-difluoroaniline (150 mg, 0.3 mmol, 1 equiv), 5-chloro-2-methoxypyridine-3-sulfonyl chloride (114 mg, 0.47 mmol, 1.5 equiv), DCM (2 mL) and pyridine (124 mg, 1.56 mmol, 5 equiv). The resulting mixture was stirred overnight at room temperature. The reaction mixture was concentrated under vacuum and the residue purified by silica gel column chromatography, eluting with PE/EtOAc (1:1) to afford 5-chloro-N-[3-[1-(4-chloro-1-[[2-(trimethylsilyl)ethoxy]methyl]imidazol-2-yl)imidazo[1,5-a]pyridin-6-yl]-2,4-difluorophenyl]-2-methoxypyridine-3-sulfonamide (140 mg, 65% yield) as a light yellow solid.

LCMS (ES, m/z): [M+H]⁺: 681.

### Synthesis of Compound 52: 5-chloro-N-[3-[1-(4-chloro-1H-imidazol-2-yl)imidazo[1,5-a]pyridin-6-yl]-2,4-difluorophenyl]-2-methoxypyridine-3-sulfonamide

Into an 8 mL vial were added 5-chloro-N-[3-[1-(4-chloro-1-[[2-(trimethylsilyl)ethoxy]methyl]imidazol-2-yl)imidazo[1,5-a]pyridin-6-yl]-2,4-difluorophenyl]-2-methoxypyridine-3-sulfonamide (130 mg, 0.19 mmol, 1 equiv) and TFA (2 mL). The resulting mixture was stirred for 2 h at 60° C., then concentrated under vacuum. The crude product was purified by Prep-HPLC with the following conditions: Column, Atlantis HILIC OBD, 19*150 mm*5 μm; Mobile Phase: 30-65% MeCN/0.1% aqueous formic acid; Flow rate: 90 mL/min; to afford 5-chloro-N-[3-[1-(4-chloro-1H-imidazol-2-yl)imidazo[1,5-a]pyridin-6-yl]-2,4-difluorophenyl]-2-methoxypyridine-3-sulfonamide (33 mg, 31% yield) as a white solid.

LCMS (ES, m/z): [M+H]⁺: 551.

¹H NMR (300 MHz, Methanol-d₄) δ 8.43-8.32 (m, 3H), 8.16 (d, J=9.5 Hz, 1H), 8.10 (d, J=2.6 Hz, 1H), 7.54 (td, J=8.9, 5.7 Hz, 1H), 7.19-7.07 (m, 2H), 6.92 (dd, J=9.4, 1.6 Hz, 1H), 4.03 (s, 3H).

### Example 68: Synthesis of 5-cyano-N-[2,4-difluoro-3-[1-(1-methylpyrazol-3-yl)imidazo[1,5-a]pyridin-6-yl]phenyl]-2-methoxypyridine-3-sulfonamide (Compound

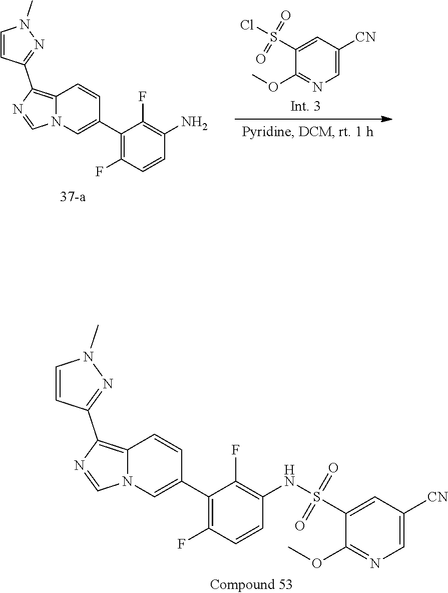 [SMILES: Cn1ccc(-c2ncn3cc(-c4c(F)ccc(N)c4F)ccc23)n1 ; COc1ncc(C#N)cc1S(=O)(=O)Cl ; COc1ncc(C#N)cc1S(=O)(=O)Nc1ccc(F)c(-c2ccc3c(-c4ccn(C)n4)ncn3c2)c1F]

### Synthesis of Compound 53: 5-cyano-N-[2,4-difluoro-3-[1-(1-methylpyrazol-3-yl)imidazo[1,5-a]pyridin-6-yl]phenyl]-2-methoxypyridine-3-sulfonamide

Into an 8 mL vial was placed 2,4-difluoro-3-[1-(1-methylpyrazol-3-yl)imidazo[1,5-a]pyridin-6-yl]aniline (80 mg, 0.25 mmol, 1 equiv), pyridine (2 mL), 5-cyano-2-methoxypyridine-3-sulfonyl chloride (172 mg, 0.7 mmol, 3 equiv) and DCM (0.1 mL). The resulting solution was stirred for 1 h at room temperature, then concentrated under vacuum. The residue was purified by prep-HPLC with the following conditions: Column, welch Vltimate XB-C18, 50×250 mm, 10 μm; Mobile Phase: 10-60% MeCN/0.1% aqueous formic acid; to afford 5-cyano-N-[2,4-difluoro-3-[1-(1-methylpyrazol-3-yl)imidazo[1,5-a]pyridin-6-yl]phenyl]-2-methoxypyridine-3-sulfonamide (34 mg, 27% yield) as a light yellow solid.

LCMS (ES, m/z): [M+H]⁺: 522

¹H NMR (300 MHz, DMSO-d₆) δ 10.52 (s, 1H), 8.93 (d, J=2.0 Hz, 1H), 8.53-8.41 (m, 3H), 8.10 (d, J=9.4 Hz, 1H), 7.73 (d, J=2.2 Hz, 1H), 7.37 (td, J=8.7, 5.6 Hz, 1H), 7.22 (t, J=9.0 Hz, 1H), 6.76 (d, J=9.4 Hz, 1H), 6.62 (d, J=2.1 Hz, 1H), 4.02 (s, 3H), 3.92 (s, 3H).

### Example 69: Synthesis of N-[2,4-difluoro-3-[1-(1-methylpyrazol-3-yl)imidazo[1,5-a]pyridin-6-yl]phenyl]-5-fluoro-2-methylpyridine-3-sulfonamide (Compound 54)

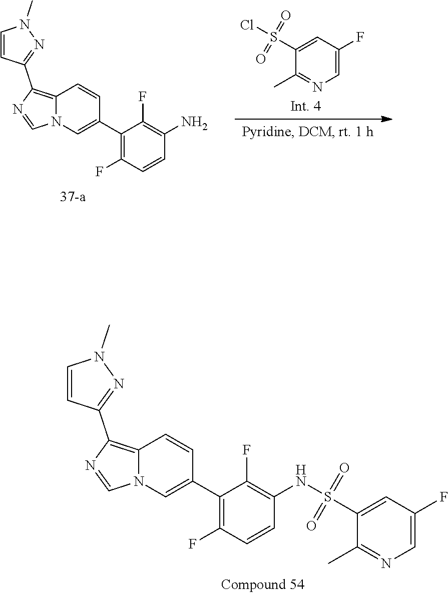 [SMILES: Cn1ccc(-c2ncn3cc(-c4c(F)ccc(N)c4F)ccc23)n1 ; Cc1ncc(F)cc1S(=O)(=O)Cl ; Cc1ncc(F)cc1S(=O)(=O)Nc1ccc(F)c(-c2ccc3c(-c4ccn(C)n4)ncn3c2)c1F]

### Synthesis of Compound 54: N-[2,4-difluoro-3-[1-(1-methylpyrazol-3-yl)imidazo[1,5-a]pyridin-6-yl]phenyl]-5-fluoro-2-methylpyridine-3-sulfonamide

Into an 8 mL vial was placed 2,4-difluoro-3-[1-(1-methylpyrazol-3-yl)imidazo[1,5-a]pyridin-6-yl]aniline (80 mg, 0.25 mmol, 1 equiv), pyridine (2 mL), 5-fluoro-2-methylpyridine-3-sulfonyl chloride (155 mg, 0.7 mmol, 3 equiv) and DCM (0.1 mL). The resulting solution was stirred for 1 h at room temperature and concentrated under vacuum. The residue was purified by prep-HPLC with the following conditions: Column, welch Vltimate XB-C18, 50×250 mm, 10 μm; Mobile Phase: 15-45% MeCN/0.1% aqueous formic acid; to afford N-[2,4-difluoro-3-[1-(1-methylpyrazol-3-yl)imidazo[1,5-a]pyridin-6-yl]phenyl]-5-fluoro-2-methylpyridine-3-sulfonamide (35 mg, 29% yield) as a light yellow solid.

LCMS (ES, m/z): [M+H]⁺: 499

¹H NMR (300 MHz, DMSO-d₆) δ 10.77 (s, 1H), 8.74 (dd, J=2.9, 1.2 Hz, 1H), 8.45 (d, J=3.1 Hz, 2H), 8.09 (d, J=9.4 Hz, 1H), 8.01-7.91 (m, 1H), 7.73 (t, J=1.7 Hz, 1H), 7.35 (td, J=8.9, 5.8 Hz, 1H), 7.24 (t, J=9.2 Hz, 1H), 6.72 (dd, J=9.5, 1.7 Hz, 1H), 6.61 (t, J=1.7 Hz, 1H), 3.92 (d, J=1.3 Hz, 3H), 2.78 (d, J=1.4 Hz, 3H).

### Example 70: Synthesis of 5-chloro-N-[2,4-difluoro-3-[1-(1,2,3-triazol-1-yl)imidazo[1,5-a]pyridin-6-yl]phenyl]-2-methoxypyridine-3-sulfonamide & 5-chloro-N-[2,4-difluoro-3-[1-(1,2,3-triazol-2-yl)imidazo[1,5-a]pyridin-6-yl]phenyl]-2-methoxypyridine-3-sulfonamide (Compound 55-1 and 55-2)

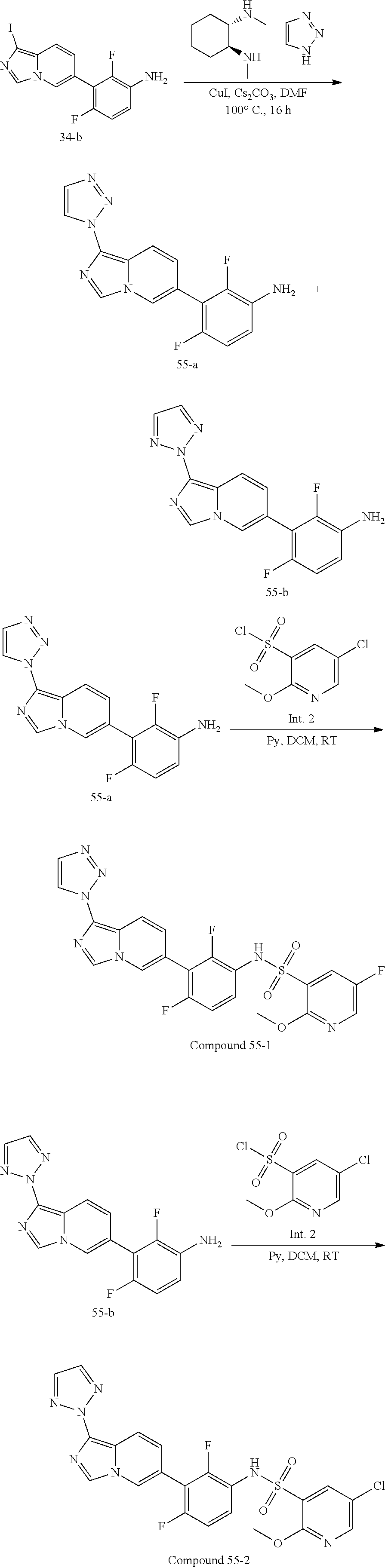

### Synthesis of 55-a/b: 2,4-difluoro-3-[1-(1,2,3-triazol-1-yl)imidazo[1,5-a]pyridin-6-yl]aniline & 2,4-difluoro-3-[1-(1,2,3-triazol-2-yl)imidazo[1,5-a]pyridin-6-yl]aniline

A mixture of 2,4-difluoro-3-[1-iodoimidazo[1,5-a]pyridin-6-yl]aniline (100 mg, 0.27 mmol, 1 equiv), (1S,2S)—N1,N2-dimethylcyclohexane-1,2-diamine (15 mg, 0.1 mmol, 0.4 equiv), 1,2,3-triazole (37 mg, 0.54 mmol, 2 equiv), Cs₂CO₃ (351 mg, 1.1 mmol, 4 equiv) and CuI (10 mg, 0.054 mmol, 0.2 equiv) in DMF (1 mL) was stirred overnight at 100° C. under nitrogen atmosphere. The mixture was allowed to cool and was diluted with water (5 mL). The resulting mixture was extracted with EtOAc (3×10 mL), and the combined organics were washed with water and brine, then dried over anhydrous Na₂SO₄, before being concentrated under reduced pressure. The crude product was purified by Prep-HPLC with the following conditions: Column, Atlantis HILIC OBD, 19*150 mm*5 μm; mobile phase: 10-42% MeCN/0.1% aqueous formic acid; Detector, uv. This resulted in 2,4-difluoro-3-[1-(1,2,3-triazol-1-yl)imidazo[1,5-a]pyridin-6-yl]aniline (13 mg, 15% yield) as an off-white solid, and 2,4-difluoro-3-[1-(1,2,3-triazol-2-yl)imidazo[1,5-a]pyridin-6-yl]aniline (15 mg, 18% yield) as an off-white solid.

LCMS (ES, m/z): [M+H]⁺: 313 & [M+H]⁺: 313.

### Synthesis of Compound 55-1: 5-chloro-N-[2,4-difluoro-3-[1-(1,2,3-triazol-1-yl)imidazo[1,5-a]pyridin-6-yl]phenyl]-2-methoxypyridine-3-sulfonamide

To a stirred solution of 2,4-difluoro-3-[1-(1,2,3-triazol-1-yl)imidazo[1,5-a]pyridin-6-yl]aniline (13 mg, 0.04 mmol, 1 equiv) and pyridine (13 mg, 0.16 mmol, 4 equiv) in DCM (0.2 mL) was added 5-chloro-2-methoxypyridine-3-sulfonyl chloride (30 mg, 0.12 mmol, 3 equiv) dropwise at room temperature and the resulting mixture was stirred for 2 h. The reaction was diluted with water (2 mL), and was extracted with EtOAc (3×10 mL). The combined organics were washed with water and brine, dried over anhydrous Na₂SO₄, then concentrated under reduced pressure. The crude product was purified by Prep-HPLC with the following conditions: Column, Atlantis HILIC OBD, 19*150 mm*5 μm; mobile phase: 25-60% MeCN/0.1% aqueous formic acid; Detector, uv. This resulted in 5-chloro-N-[2,4-difluoro-3-[1-(1,2,3-triazol-1-yl)imidazo[1,5-a]pyridin-6-yl]phenyl]-2-methoxypyridine-3-sulfonamide (2.8 mg, 13% yield) as an off-white solid.

LCMS (ES, m/z): [M+H]⁺: 518.

¹H NMR (300 MHz, DMSO-d6) δ 10.46 (s, 1H), 8.68 (d, J=1.2 Hz, 1H), 8.60 (d, J=4.8 Hz, 2H), 8.50 (s, 1H), 8.09 (d, J=2.6 Hz, 1H), 8.02-7.92 (m, 2H), 7.37 (s, 1H), 7.26 (d, J=9.7 Hz, 1H), 6.95 (d, J=9.9 Hz, 1H), 3.92 (s, 3H).

### Synthesis of Compound 55-2: 5-chloro-N-[2,4-difluoro-3-[1-(1,2,3-triazol-2-yl)imidazo[1,5-a]pyridin-6-yl]phenyl]-2-methoxypyridine-3-sulfonamide

To a stirred solution of 2,4-difluoro-3-[1-(1,2,3-triazol-2-yl)imidazo[1,5-a]pyridin-6-yl]aniline (15 mg, 0.045 mmol, 1 equiv) and pyridine (15 mg, 0.18 mmol, 4 equiv) in DCM (0.1 mL) was added 5-chloro-2-methoxypyridine-3-sulfonyl chloride (11 mg, 0.05 mmol, 1.5 equiv) dropwise at room temperature. The resulting mixture was stirred for 2 h, then was diluted with water (2 mL). The resulting mixture was extracted with EtOAc (3×10 mL) and the combined organics were washed with water and brine, then dried over anhydrous Na₂SO₄, before being concentrated under reduced pressure. The crude product was purified by Prep-HPLC with the following conditions: Column, Atlantis HILIC OBD, 19*150 mm*5 μm; mobile phase: 30-70% MeCN/0.1% aqueous formic acid; Detector, uv. This resulted in 5-chloro-N-[2,4-difluoro-3-[1-(1,2,3-triazol-2-yl)imidazo[1,5-a]pyridin-6-yl]phenyl]-2-methoxypyridine-3-sulfonamide (9.1 mg, 55% yield) as an off-white solid.

LCMS (ES, m/z): [M+H]⁺: 518.

¹H-NMR (300 MHz, DMSO-d6) δ 10.46 (s, 1H), 8.59 (s, 1H), 8.55-8.47 (m, 2H), 8.13 (s, 2H), 8.10 (d, J=2.6 Hz, 1H), 7.96 (d, J=9.5 Hz, 1H), 7.46-7.34 (m, 1H), 7.28 (dd, J=9.1, 1.5 Hz, 1H), 6.91 (dd, J=9.6, 1.6 Hz, 1H), 3.93 (s, 3H).

### Example 71: Synthesis of 5-chloro-N-[2,4-difluoro-3-[1-(4-methyl-3H-imidazol-2-yl)imidazo[1,5-a]pyridine-6-yl]phenyl]-2-methylpyridine-3-sulfonamide (Compound 56)

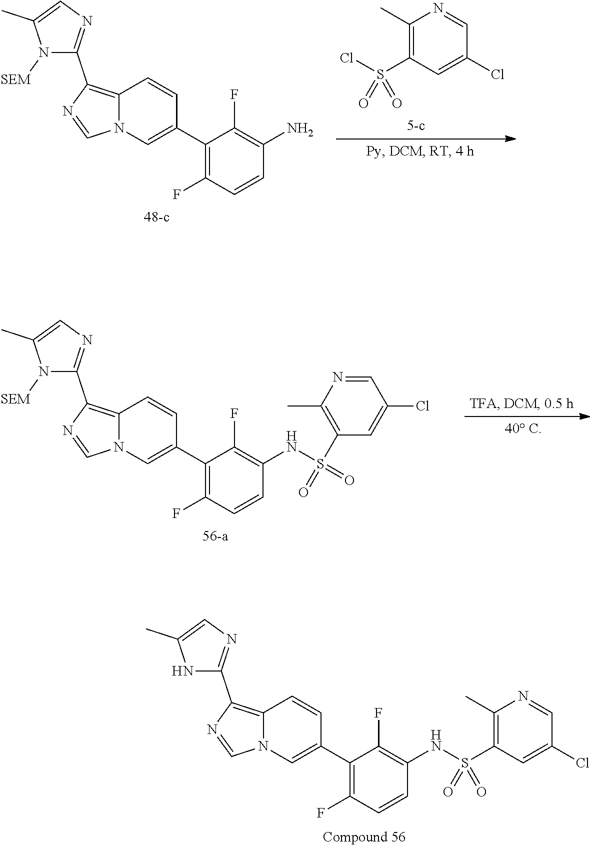 [SMILES: Cc1cnc(-c2ncn3cc(-c4c(F)ccc(N)c4F)ccc23)n1COCC[Si](C)(C)C ; Cc1ncc(Cl)cc1S(=O)(=O)Cl ; Cc1ncc(Cl)cc1S(=O)(=O)Nc1ccc(F)c(-c2ccc3c(-c4ncc(C)n4COCC[Si](C)(C)C)ncn3c2)c1F ; Cc1cnc(-c2ncn3cc(-c4c(F)ccc(NS(=O)(=O)c5cc(Cl)cnc5C)c4F)ccc23)[nH]1]

### Synthesis of 56-a: 5-chloro-N-[2,4-difluoro-3-[1-(5-methyl-1-[[2-(trimethylsilyl)ethoxy]methyl]imidazol-2-yl)imidazo[1,5-a]pyridin-6-yl]phenyl]-2-methylpyridine-3-sulfonamide

To a stirred solution of 2,4-difluoro-3-[1-(5-methyl-1-[[2-(trimethylsilyl)ethoxy]methyl]imidazol-2-yl)imidazo[1,5-a]pyridin-6-yl]aniline (200 mg, 0.44 mmol, 1 equiv) and pyridine (139 mg, 1.76 mmol, 4 equiv) in DCM (2 mL) was added 5-chloro-2-methylpyridine-3-sulfonyl chloride (200 mg, 0.88 mmol, 2 equiv) in portions, and the reaction stirred for 4 h at room temperature. Water (2 mL) was added and the resulting mixture was extracted with CH₂Cl₂ (3×10 mL). The combined organics were washed with water and brine, dried over anhydrous Na₂SO₄ and concentrated under reduced pressure. The residue was purified by silica gel column chromatography, eluting with EtOAc/PE (0-50%) to afford 5-chloro-N-[2,4-difluoro-3-[1-(5-methyl-1-[[2-(trimethylsilyl)ethoxy]methyl]imidazol-2-yl)imidazo[1,5-a]pyridin-6-yl]phenyl]-2-methylpyridine-3-sulfonamide (190 mg, 67% yield) as a yellow solid.

LCMS (ES, m/z): [M+H]⁺: 645.

### Synthesis of Compound 56: 5-chloro-N-[2,4-difluoro-3-[1-(4-methyl-3H-imidazol-2-yl)imidazo[1,5-a]pyridine-6-yl]phenyl]-2-methylpyridine-3-sulfonamide

To a stirred mixture of 5-chloro-N-[2,4-difluoro-3-[1-(5-methyl-1-[[2-(trimethylsilyl)ethoxy]methyl]imidazol-2-yl)imidazo[1,5-a]pyridin-6-yl]phenyl]-2-methylpyridine-3-sulfonamide (160 mg, 0.25 mmol, 1 equiv) was added DCM (2 mL) and TFA (1 mL). The resulting mixture was stirred for 30 min at 40° C., then concentrated under vacuum. The residue was neutralized to pH 7 with ammonia, and was purified by Prep-HPLC: Column, Atlantis HILIC OBD, 19*150 mm*5 μm; Mobile Phase: 10-50% MeCN/0.1% aqueous formic acid; Flow rate: 90 mL/min; to afford 5-chloro-N-[2,4-difluoro-3-[1-(4-methyl-3H-imidazol-2-yl)imidazo[1,5-a]pyridin-6-yl]phenyl]-2-methylpyridine-3-sulfonamide (11 mg, 9% yield) as a light yellow solid.

LCMS (ES, m/z): [M+H]⁺: 515.

¹H NMR (300 MHz, DMSO-d₆): 11.91 (br, 1H), 8.73 (d, J=2.4 Hz, 1H), 8.52 (d, J=5.0 Hz, 2H), 8.25-8.11 (m, 1H), 8.08 (d, J=2.4 Hz, 1H), 7.37-7.32 (m, 1H), 7.20 (td, J=9.2, 1.6 Hz, 1H), 6.89-6.80 (m, 2H), 2.77 (s, 3H), 2.23 (s, 3H).

### Example 72: Synthesis of 5-chloro-N-[2,4-difluoro-3-[1-(5-methyl-4H-1,2,4-triazol-3-yl)imidazo[1,5-a]pyridin-6-yl]phenyl]-2-methoxypyridine-3-sulfonamide (Compound

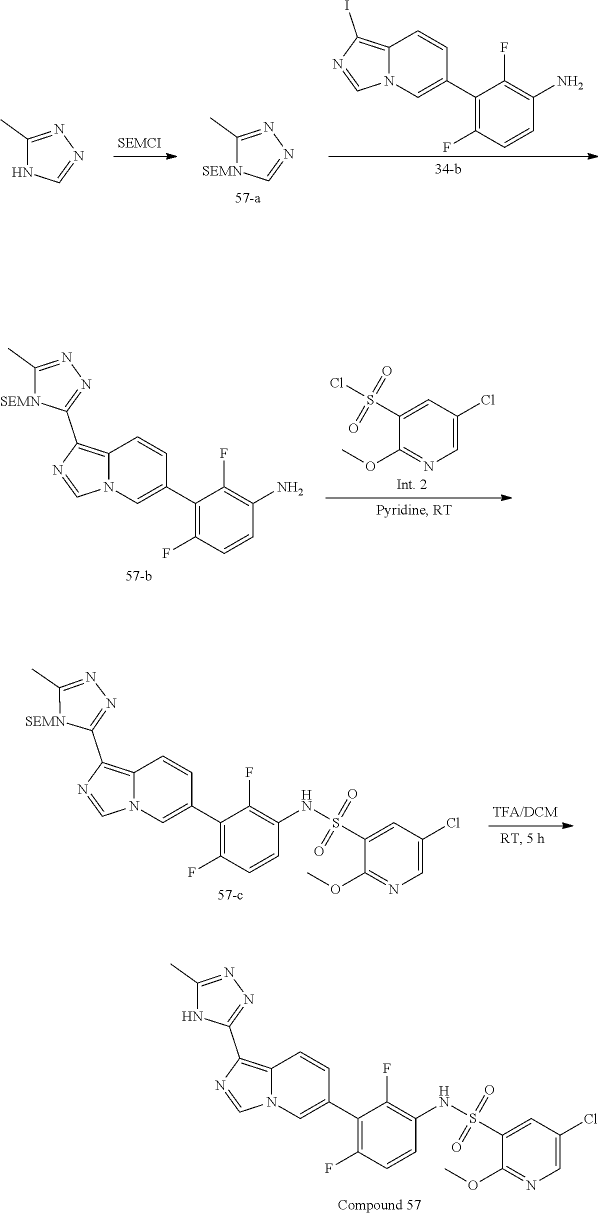

### Synthesis of 57-a: 3-methyl-4-[[2-(trimethylsilyl)ethoxy]methyl]-1,2,4-triazole

To a solution of 3-methyl-4H-1,2,4-triazole (3 g, 36 mmol, 1 equiv) in DMF (180 mL) was added NaH (2.9 g, 72 mmol, 2 equiv, 60% in oil) in portions at 0° C. The resulting mixture was stirred for 0.5 h at 0° C., then SEM-Cl (7.2 g, 43.32 mmol, 1.2 equiv) was added dropwise at 0° C. The resulting solution was stirred for 2 h at room temperature, then quenched with 100 mL of water. The resulting solution was extracted with 3×100 mL of ethyl acetate and the organics dried over anhydrous sodium sulfate, before being concentrated under vacuum. The residue was applied to a silica gel column, eluting with ethyl acetate/petroleum ether (1:1). This resulted in 3-methyl-4-[[2-(trimethylsilyl)ethoxy]methyl]-1,2,4-triazole (3 g, 39% yield) as an off-white liquid.

LCMS (ES, m/z): [M+H]⁺: 214

### Synthesis of 57-b: 2,4-difluoro-3-[1-(5-methyl-4-[[2-(trimethylsilyl)ethoxy]methyl]-1,2,4-triazol-3-yl)imidazo[1,5-a]pyridin-6-yl]aniline

Into a 100 mL 3-necked round-bottom flask, purged and maintained with an inert atmosphere of nitrogen, was placed 3-methyl-4-[[2-(trimethylsilyl)ethoxy]methyl]-1,2,4-triazole (1.2 g, 5.7 mmol, 3 equiv) in THE (35 mL) and the mixture was cooled to −78° C. 2.5M n-BuLi in hexanes (2.5 mL, 6.2 mmol, 3.3 equiv) was added dropwise at −78° C. for 5 min, and the resulting solution was stirred for 30 min at −78° C. ZnCl₂ (1 M in Et₂O, 6.2 mL, 6.2 mmol, 3.3 equiv) was added at low temperature, then the mixture was warmed to room temperature for 30 min. A solution of Pd(PPh₃)₄ (0.22 g, 0.19 mmol, 0.1 equiv) in THE (1 mL) and a solution of 2,4-difluoro-3-[1-iodoimidazo[1,5-a]pyridin-6-yl]aniline (0.7 g, 1.88 mmol, 1 equiv) in THE (2 mL) were added. The resulting solution was heated to 60° C. overnight. The reaction was cooled and quenched by the addition of 100 mL of water. The solids were removed by filtration, and the filtrate extracted with 3×50 mL of ethyl acetate. The combined organics were washed with 100 ml of brine, dried over anhydrous sodium sulfate and concentrated under vacuum. The crude product was purified by Flash-Prep-HPLC with the following conditions: Column, WelFlash™ C18-I, Spherical C18 20-40 m, 120 g; mobile phase: 10-70% MeCN/0.1% formic acid; Detector, 220 nm; to give 2,4-difluoro-3-[1-(5-methyl-4-[[2-(trimethylsilyl)ethoxy]methyl]-1,2,4-triazol-3-yl)imidazo[1,5-a]pyridin-6-yl]aniline (0.65 g, 75% yield) as a light brown semi-solid.

LCMS (ES, m/z): [M+H]⁺: 457

### Synthesis of 57-c: 5-chloro-N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-2-methoxypyridine-3-sulfonamide

Into a 40 mL vial was placed 2,4-difluoro-3-[1-(5-methyl-4-[[2-(trimethylsilyl)ethoxy]methyl]-1,2,4-triazol-3-yl)imidazo[1,5-a]pyridin-6-yl]aniline (100 mg, 0.22 mmol, 1 equiv), pyridine (3 mL) and 5-chloro-2-methoxypyridine-3-sulfonyl chloride (80 mg, 0.33 mmol, 1.5 equiv). The resulting solution was stirred overnight at room temperature, then concentrated under reduced pressure. The residue was purified by Flash-Prep-HPLC with the following conditions: Column, WelFlash™ C18-I, Spherical C18 20-m, 120 g; mobile phase; 10-85% MeCN/0.1% aqueous formic acid; Detector, 220 nm; to yield 5-chloro-N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-2-methoxypyridine-3-sulfonamide (86 mg, 59% yield) as a white solid.

LCMS (ES, m/z): [M+H]⁺: 662

### Synthesis of Compound 57: 5-chloro-N-[2,4-difluoro-3-[1-(5-methyl-4H-1,2,4-triazol-3-yl)imidazo[1,5-a]pyridin-6-yl]phenyl]-2-methoxypyridine-3-sulfonamide

Into a 40 mL vial was placed 5-chloro-N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-2-methoxypyridine-3-sulfonamide (85 mg, 0.13 mmol, 1 equiv), DCM (3 mL) and TFA (1 mL). The resulting solution was stirred for 5 h at room temperature, then concentrated under vacuum. The pH was adjusted to 8 with NH₃ (7 mol/L in MeOH, 3 mL) and the crude product was purified by Prep-HPLC using the following conditions: Column, welch Vltimate XB-C18, 50×250 mm, 10 μm; Mobile Phase 15-55% MeCN/0.1% aqueous formic acid; Detector, 220 nm; to give 5-chloro-N-[2,4-difluoro-3-[1-(5-methyl-4H-1,2,4-triazol-3-yl)imidazo[1,5-a]pyridin-6-yl]phenyl]-2-methoxypyridine-3-sulfonamide (48 mg, 70% yield) as a white solid.

LCMS (ES, m/z): [M+H]⁺: 532

¹H NMR (300 MHz, DMSO-d₆) δ 14.00-13.54 (m, 1H), 10.46 (s, 1H), 8.70-8.44 (m, 3H), 8.21 (d, J=9.4 Hz, 1H), 8.09 (d, J=2.6 Hz, 1H), 7.42-7.31 (m, 1H), 7.25 (t, J=9.1 Hz, 1H), 7.04-6.78 (m, 1H), 3.93 (s, 3H), 2.38 (m, 3H).

### Example 73: Synthesis of N-[2,4-difluoro-3-[1-(5-methyl-4H-1,2,4-triazol-3-yl)imidazo[1,5-a]pyridin-6-yl]phenyl]-5-fluoro-2-methoxypyridine-3-sulfonamide (Compound 58)

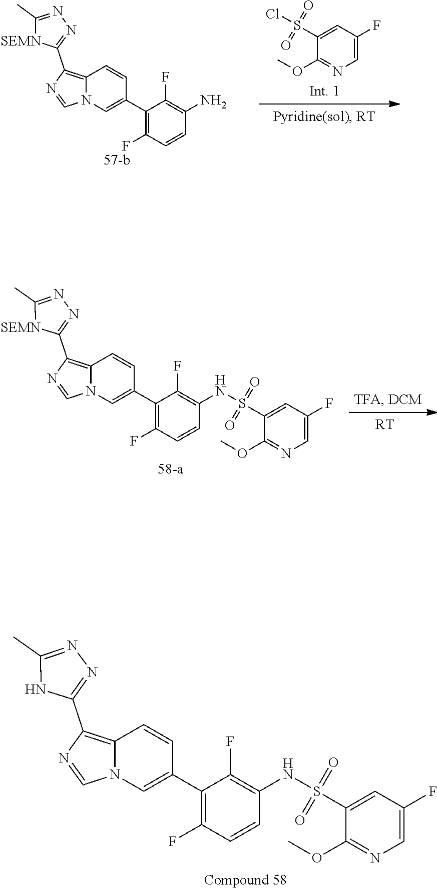 [SMILES: Cc1nnc(-c2ncn3cc(-c4c(F)ccc(N)c4F)ccc23)n1COCC[Si](C)(C)C ; COc1ncc(F)cc1S(=O)(=O)Cl ; COc1ncc(F)cc1S(=O)(=O)Nc1ccc(F)c(-c2ccc3c(-c4nnc(C)n4COCC[Si](C)(C)C)ncn3c2)c1F ; "RT" ; COc1ncc(F)cc1S(=O)(=O)Nc1ccc(F)c(-c2ccc3c(-c4nnc(C)[nH]4)ncn3c2)c1F]

### Synthesis of 58-a: N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-5-fluoro-2-methoxypyridine-3-sulfonamide

Into a 40 mL vial was placed 2,4-difluoro-3-[1-(5-methyl-4-[[2-(trimethylsilyl)ethoxy]methyl]-1,2,4-triazol-3-yl)imidazo[1,5-a]pyridin-6-yl]aniline (100 mg, 0.22 mmol, 1 equiv), pyridine (4 mL) and 5-fluoro-2-methoxypyridine-3-sulfonyl chloride (74 mg, 0.33 mmol, 1.5 equiv). The resulting solution was stirred overnight at 25° C. and concentrated under reduced pressure. The residue was purified by Flash-Prep-HPLC with the following conditions: Column, WelFlash™ C18-I, Spherical C18 20-40 m, 120 g; mobile phase: 5-90% MeCN/0.1% aqueous formic acid; Detector, 220 nm; to give N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-5-fluoro-2-methoxypyridine-3-sulfonamide (80 mg, 57% yield) as a white solid.

LCMS (ES, m/z): [M+H]⁺: 646

### Synthesis of Compound 58: N-[2,4-difluoro-3-[1-(5-methyl-4H-1,2,4-triazol-3-yl)imidazo[1,5-a]pyridin-6-yl]phenyl]-5-fluoro-2-methoxypyridine-3-sulfonamide

Into a 40 mL vial was placed N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-5-fluoro-2-methoxypyridine-3-sulfonamide (80 mg, 1 equiv), DCM (3 mL) and TFA (1 mL).

The resulting solution was stirred for 5 h at room temperature, then concentrated under vacuum. The pH was adjusted to 8 with NH₃ (7 mol/L in MeOH, 3 mL) and the crude product was purified by Prep-HPLC with the following conditions: Column, welch Vltimate XB-C18, 50×250 mm, 10 μm; Mobile Phase: 13-48% MeCN/0.1% aqueous formic acid; Detector, 220 nm; to yield N-[2,4-difluoro-3-[1-(5-methyl-4H-1,2,4-triazol-3-yl)imidazo[1,5-a]pyridin-6-yl]phenyl]-5-fluoro-2-methoxypyridine-3-sulfonamide (23 mg, 35% yield) as a white solid.

LCMS (ES, m/z): [M+H]⁺: 516 H NMR (300 MHz, DMSO-d₆) δ 13.92-13.50 (d, 1H), 10.45 (s, 1H), 8.60 (s, 1H), 8.56-8.44 (m, 2H), 8.21 (d, J=9.4 Hz, 1H), 8.03 (dd, J=7.3, 3.0 Hz, 1H), 7.37 (q, J=7.9, 7.3 Hz, 1H), 7.25 (t, J=9.1 Hz, 1H), 7.02-6.77 (m, 1H), 3.92 (s, 3H), 2.43-2.33 (m, 3H).

### Example 74: Synthesis of 5-cyano-N-[2,4-difluoro-3-[1-(5-methyl-4H-1,2,4-triazol-3-yl)imidazo[1,5-a]pyridin-6-yl]phenyl]-2-methoxypyridine-3-sulfonamide (Compound 59)

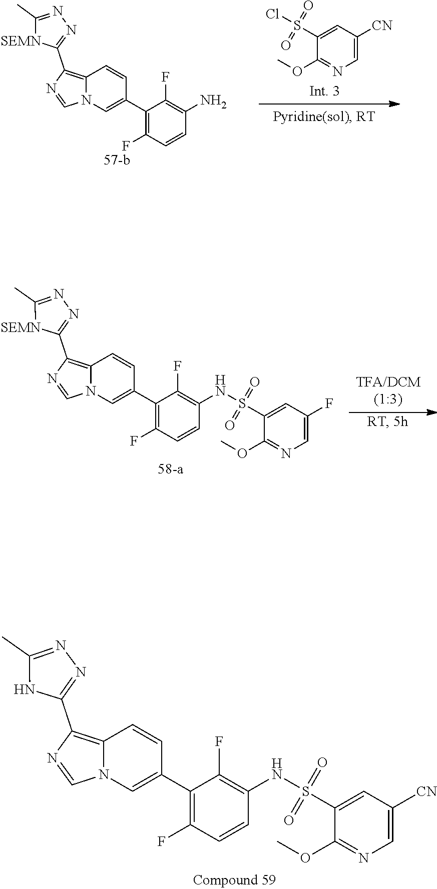 [SMILES: Cc1nnc(-c2ncn3cc(-c4c(F)ccc(N)c4F)ccc23)n1COCC[Si](C)(C)C ; COc1ncc(C#N)cc1S(=O)(=O)Cl ; COc1ncc(F)cc1S(=O)(=O)Nc1ccc(F)c(-c2ccc3c(-c4nnc(C)n4COCC[Si](C)(C)C)ncn3c2)c1F ; "RT, 5h" ; COc1ncc(C#N)cc1S(=O)(=O)Nc1ccc(F)c(-c2ccc3c(-c4nnc(C)[nH]4)ncn3c2)c1F]

### Synthesis of 59-a: 5-cyano-N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-2-methoxypyridine-3-sulfonamide

Into a 40 mL vial, was placed 2,4-difluoro-3-[1-(5-methyl-4-[[2-(trimethylsilyl)ethoxy]methyl]-1,2,4-triazol-3-yl)imidazo[1,5-a]pyridin-6-yl]aniline (100 mg, 0.22 mmol, 1 equiv), pyridine (4 mL) and 5-cyano-2-methoxypyridine-3-sulfonyl chloride (76 mg, 0.33 mmol, 1.5 equiv). The resulting solution was stirred overnight at room temperature. The reaction mixture was concentrated under reduced pressure. The residue was purified by Flash-Prep-HPLC with the following conditions: Column, WelFlash™ C18-I, Spherical C18 20-40 m, 120 g; mobile phase, 10-90% MeCN/0.1% aqueous formic acid over 15 mins; Detector, 220 nm. This resulted in 5-cyano-N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-2-methoxypyridine-3-sulfonamide (95 mg, 66%) as a white solid.

LCMS (ES, m/z): [M+H]⁺: 653

### Synthesis of Compound 59: 5-cyano-N-[2,4-difluoro-3-[1-(5-methyl-4H-1,2,4-triazol-3-yl)imidazo[1,5-a]pyridin-6-yl]phenyl]-2-methoxypyridine-3-sulfonamide

Into a 40 mL vial, was placed 5-cyano-N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-2-methoxypyridine-3-sulfonamide (90 mg, 0.14 mmol, 1 equiv), DCM (3 mL) and TFA (1 mL). The resulting solution was stirred for 5 h at room temperature. The resulting mixture was concentrated under vacuum. The pH value of the solution was adjusted to 8 with NH₃ (7 mol/L in MeOH, 3 mL). The crude product was purified by Prep-HPLC with the following conditions: Column, welch Vltimate XB-C18, 50×250 mm, 10 μm mobile phase; Mobile Phase, 10-47% MeCN/0.1% aqueous formic acid over 15 mins; Detector, 220 nm. This resulted in 5-cyano-N-[2,4-difluoro-3-[1-(5-methyl-4H-1,2,4-triazol-3-yl)imidazo[1,5-a]pyridin-6-yl]phenyl]-2-methoxypyridine-3-sulfonamide (21 mg, 29%) as a light yellow solid.

LCMS (ES, m/z): [M+H]⁺: 523

¹H NMR (300 MHz, DMSO-d₆) δ 13.95-13.50 (m, 1H), 10.45 (s, 1H), 8.91 (d, J=2.2 Hz, 1H), 8.59 (s, 1H), 8.48 (d, J=2.2 Hz, 2H), 8.25-8.11 (m, 1H), 7.36 (td, J=8.9, 5.9 Hz, 1H), 7.20 (t, J=9.1 Hz, 1H), 7.03-6.75 (m, 1H), 4.01 (s, 3H), 2.42-2.33 (m, 3H).

### Example 75: Synthesis of: N-[2,4-difluoro-3-[1-(5-methyl-4H-1,2,4-triazol-3-yl)imidazo[1,5-a]pyridin-6-yl]phenyl]-5-fluoro-2-methylpyridine-3-sulfonamide (Compound 60)

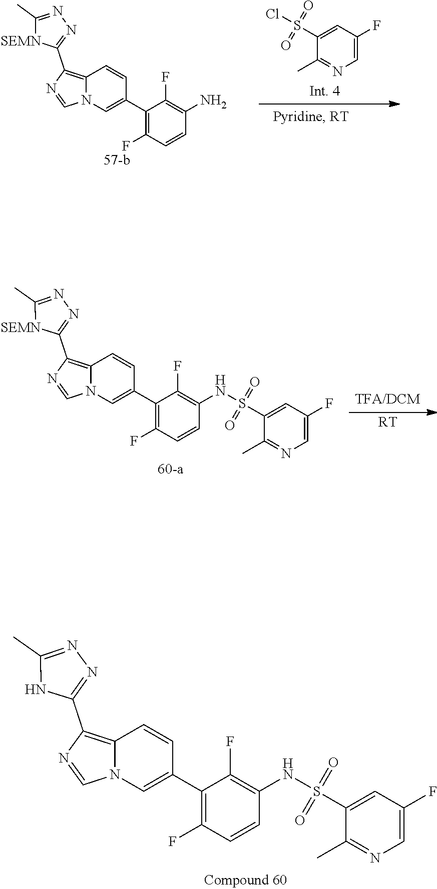 [SMILES: Cc1nnc(-c2ncn3cc(-c4c(F)ccc(N)c4F)ccc23)n1COCC[Si](C)(C)C ; Cc1ncc(F)cc1S(=O)(=O)Cl ; Cc1ncc(F)cc1S(=O)(=O)Nc1ccc(F)c(-c2ccc3c(-c4nnc(C)n4COCC[Si](C)(C)C)ncn3c2)c1F ; "RT" ; Cc1nnc(-c2ncn3cc(-c4c(F)ccc(NS(=O)(=O)c5cc(F)cnc5C)c4F)ccc23)[nH]1]

### Synthesis of 60-a: N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-5-fluoro-2-methylpyridine-3-sulfonamide

Into a 40 mL vial was placed 2,4-difluoro-3-[1-(5-methyl-4-[[2-(trimethylsilyl)ethoxy]methyl]-1,2,4-triazol-3-yl)imidazo[1,5-a]pyridin-6-yl]aniline (400 mg, 0.88 mmol, 1 equiv), pyridine (10 mL) and 5-fluoro-2-methylpyridine-3-sulfonyl chloride (367 mg, 1.8 mmol, 2 equiv). The resulting solution was stirred overnight at 25° C. Concentration gave the crude product which was purified by Flash-Prep-HPLC using the following conditions: Column, WelFlash™ C18-I, Spherical C18 20-40 m, 120 g; mobile phase: 10-89% MeCN/0.1% aqueous formic acid; Detector, 220 nm; to give N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-5-fluoro-2-methylpyridine-3-sulfonamide (370 mg, 67% yield) as a white solid.

LCMS (ES, m/z): [M+H]⁺: 630

### Synthesis of Compound 60: N-[2,4-difluoro-3-[1-(5-methyl-4H-1,2,4-triazol-3-yl)imidazo[1,5-a]pyridin-6-yl]phenyl]-5-fluoro-2-methylpyridine-3-sulfonamide

Into a 40 mL vial was placed N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-5-fluoro-2-methylpyridine-3-sulfonamide (360 mg, 1 equiv), DCM (10 mL) and TFA (3 mL). The resulting solution was stirred for 5 h at 25° C. and concentrated under vacuum. The pH as adjusted to 8 with NH₃ (7 mol/L in MeOH, 2 mL) and the crude product purified by Prep-HPLC with the following conditions: Column, welch Vltimate XB-C18, 50×250 mm, 10 μm; Mobile Phase 5-37% MeCN/0.05% aqueous NH₃; Detector, 220 nm; to give N-[2,4-difluoro-3-[1-(5-methyl-4H-1,2,4-triazol-3-yl)imidazo[1,5-a]pyridin-6-yl]phenyl]-5-fluoro-2-methylpyridine-3-sulfonamide (150 mg, 53% yield) as a white solid.

LCMS (ES, m/z): [M+H]⁺: 500

¹H NMR (300 MHz, DMSO-d₆) δ 13.95-13.55 (m, 1H), 10.79 (br s, 1H), 8.74 (d, J=2.8 Hz, 1H), 8.69-8.41 (m, 2H), 8.20 (d, J=9.4 Hz, 1H), 7.96 (dd, J=8.2, 2.9 Hz, 1H), 7.46-7.21 (m, 2H), 7.04-6.71 (m, 1H), 2.78 (d, J=1.2 Hz, 3H), 2.50 (s, 3H).

### Example 76: Synthesis of 5-chloro-N-[2,4-difluoro-3-[1-(5-methyl-4H-1,2,4-triazol-3-yl)imidazo[1,5-a]pyridin-6-yl]phenyl]-2-methylpyridine-3-sulfonamide (Compound 61)

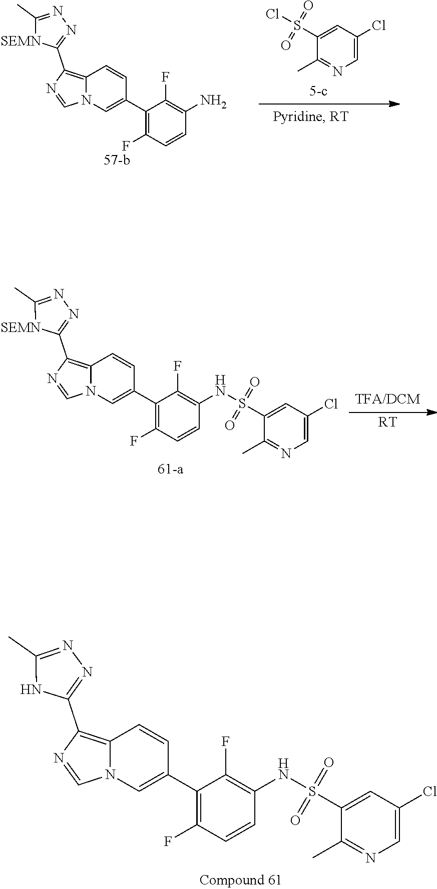 [SMILES: Cc1nnc(-c2ncn3cc(-c4c(F)ccc(N)c4F)ccc23)n1COCC[Si](C)(C)C ; Cc1ncc(Cl)cc1S(=O)(=O)Cl ; Cc1ncc(Cl)cc1S(=O)(=O)Nc1ccc(F)c(-c2ccc3c(-c4nnc(C)n4COCC[Si](C)(C)C)ncn3c2)c1F ; "RT" ; Cc1nnc(-c2ncn3cc(-c4c(F)ccc(NS(=O)(=O)c5cc(Cl)cnc5C)c4F)ccc23)[nH]1]

### Synthesis of 61-a: 5-chloro-N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-2-methylpyridine-3-sulfonamide

Into a 40 mL vial was placed 2,4-difluoro-3-[1-(5-methyl-4-[[2-(trimethylsilyl)ethoxy]methyl]-1,2,4-triazol-3-yl) imidazo[1,5-a]pyridin-6-yl]aniline (100 mg, 0.22 mmol, 1 equiv), pyridine (3 mL) and 5-chloro-2-methylpyridine-3-sulfonyl chloride (99 mg, 0.44 mmol, 2 equiv). The resulting solution was stirred overnight at 25° C., then concentrated under reduced pressure to afford 5-chloro-N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-2-methylpyridine-3-sulfonamide (93 mg, 66% yield) as a white solid which was used in the next step without further purification.

LCMS (ES, m/z): [M+H]⁺: 646

### Synthesis of Compound 61: 5-chloro-N-[2,4-difluoro-3-[1-(5-methyl-4H-1,2,4-triazol-3-yl)imidazo[1,5-a]pyridin-6-yl]phenyl]-2-methylpyridine-3-sulfonamide

Into a 40 mL vial was placed 5-chloro-N-[2,4-difluoro-3-[1-(5-methyl-4-[[2-(trimethylsilyl)ethoxy]methyl]-1,2,4-triazol-3-yl)imidazo[1,5-a]pyridin-6-yl]phenyl]-2-methylpyridine-3-sulfonamide (90 mg, 0.13 mmol, 1 equiv), DCM (3 mL) and TFA (1 mL, 13 mmol). The resulting solution was stirred for 5 h at 25° C., then concentrated under vacuum. The pH was adjusted to 8 with NH₃ (7 mol/L in MeOH, 3 mL) and the crude product was purified by Prep-HPLC with the following conditions: Column, welch Vltimate XB-C18, 50×250 mm, 10 μm; Mobile Phase, 10-50% MeCN/0.1% aqueous formic acid; Detector, 220 nm; to give 5-chloro-N-[2,4-difluoro-3-[1-(5-methyl-4H-1,2,4-triazol-3-yl)imidazo[1,5-a]pyridin-6-yl]phenyl]-2-methylpyridine-3-sulfonamide (37 mg, 51% yield) as a white solid.

LCMS (ES, m/z): [M+H]⁺: 516

¹H NMR (300 MHz, DMSO-d₆) δ 14.01-13.44 (m, 1H), 10.79 (s, 1H), 8.78 (d, J=2.4 Hz, 1H), 8.63-8.43 (m, 2H), 8.20 (d, J=9.4 Hz, 1H), 8.08 (d, J=2.4 Hz, 1H), 7.44-7.19 (m, 2H), 6.98-6.75 (m, 1H), 2.77 (s, 3H), 2.38 (s, 3H).

### Example 77: Synthesis of 5-chloro-N-[2,4-difluoro-3-[1-(1-methylpyrazol-3-yl)imidazo[1,5-a]pyridin-6-yl]phenyl]-2-methylpyridine-3-sulfonamide (Compound 62)

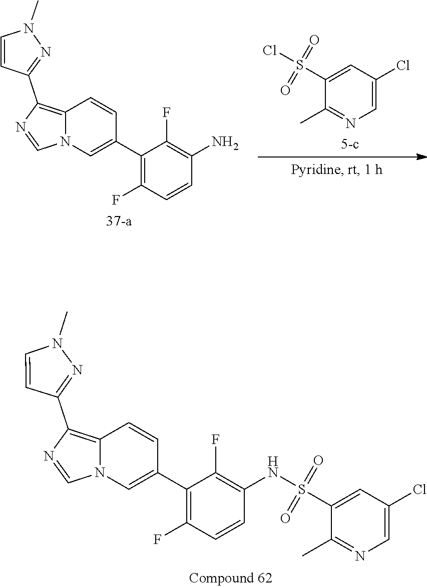 [SMILES: Cn1ccc(-c2ncn3cc(-c4c(F)ccc(N)c4F)ccc23)n1 ; Cc1ncc(Cl)cc1S(=O)(=O)Cl ; Cc1ncc(Cl)cc1S(=O)(=O)Nc1ccc(F)c(-c2ccc3c(-c4ccn(C)n4)ncn3c2)c1F]

### Synthesis of Compound 62: 5-chloro-N-[2,4-difluoro-3-[1-(1-methylpyrazol-3-yl)imidazo[1,5-a]pyridin-6-yl]phenyl]-2-methylpyridine-3-sulfonamide

Into an 8 mL vial was placed 2,4-difluoro-3-[1-(1-methylpyrazol-3-yl)imidazo[1,5-a]pyridin-6-yl]aniline (80 mg, 0.25 mmol, 1 equiv), pyridine (2 mL) and 5-chloro-2-methylpyridine-3-sulfonyl chloride (167 mg, 0.7 mmol, 3 equiv) at room temperature. The resulting solution was stirred for 1 h then concentrated under vacuum. The residue was purified by prep-HPLC with the following conditions: Column, welch Vltimate XB-C18, 50×250 mm, 10 μm; Mobile Phase 15-45% MeCN/0.1% aqueous formic acid; to afford 5-chloro-N-[2,4-difluoro-3-[1-(1-methylpyrazol-3-yl)imidazo[1,5-a]pyridin-6-yl]phenyl]-2-methylpyridine-3-sulfonamide (20 mg, 16% yield) as a light yellow solid.

LCMS (ES, m/z): [M+H]⁺: 515

¹H NMR (300 MHz, DMSO-d₆) δ 8.67 (s, 1H), 8.43 (s, 2H), 8.13-8.04 (m, 2H), 7.72 (d, J=2.2 Hz, 1H), 7.27 (q, 1H), 7.13 (t, 1H), 6.75 (d, J=9.4 Hz, 1H), 6.61 (d, J=2.2 Hz, 1H), 3.92 (s, 3H), 2.77 (s, 3H).

### Example 78: Synthesis of 5-chloro-N-[2,4-difluoro-3-[1-(1,2,3,4-tetrazol-1-yl)imidazo[1,5-a]pyridin-6-yl]phenyl]-2-methoxypyridine-3-sulfonamide (Compound

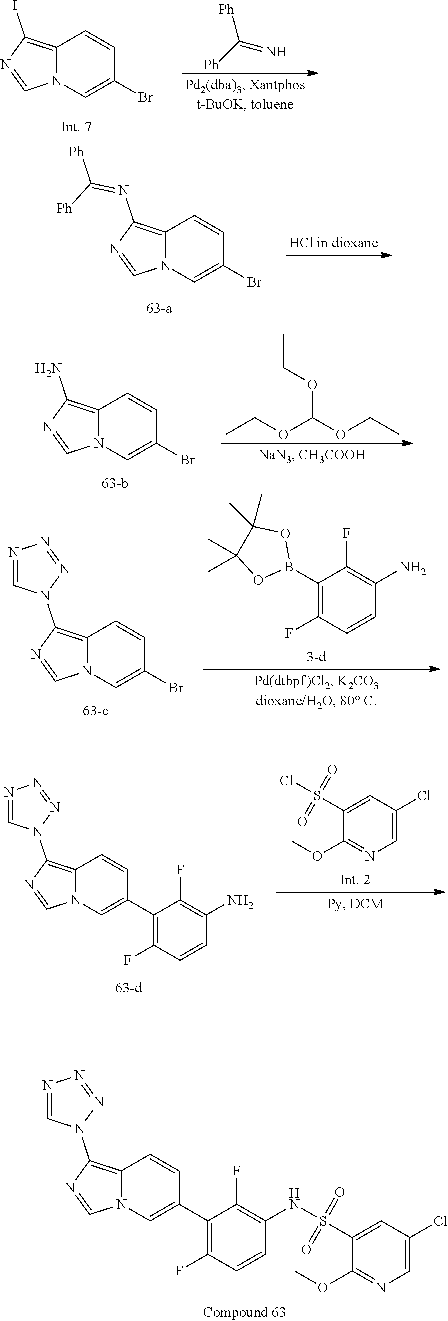

### Synthesis of 63-a: N-[6-bromoimidazo[1,5-a]pyridin-1-yl]-1,1-diphenylmethanimine

A mixture of phenyl-benzenemethanamine (1.8 g, 9.9 mmol, 0.8 equiv), 6-bromo-1-iodoimidazo[1,5-a]pyridine (4 g, 12 mmol, 1 equiv), Pd₂(dba)₃ (1.13 g, 1.2 mmol, 0.1 equiv), XantPhos (1.43 g, 2.5 mmol, 0.2 equiv) and t-BuONa (3.57 g, 37 mmol, 3 equiv) in toluene (80 mL) was stirred for 1 h at 60° C. under nitrogen atmosphere. The mixture was allowed to cool to room temperature and was diluted with water (100 mL). This was extracted with EtOAc (3×100 mL) and the combined organic layers were washed with water and brine, dried over anhydrous Na₂SO₄ and concentrated under reduced pressure. The residue was purified by silica gel column chromatography, eluting with EtOAc/PE (0-50%) to afford N-[6-bromoimidazo[1,5-a]pyridin-1-yl]-1,1-diphenylmethanimine (5 g, 86% yield) as a brown solid.

LCMS (ES, m/z): [M+H]⁺: 376, 378

### Synthesis of 63-b: 6-bromoimidazo[1,5-a]pyridin-1-amine hydrochloride

To a stirred mixture of N-[6-bromoimidazo[1,5-a]pyridin-1-yl]-1,1-diphenylmethanimine (800 mg, 2.1 mmol, 1 equiv) were added HCl in 1,4-dioxane (8 mL, 32 mmol, 15 equiv, 4 M) dropwise at 0° C. The resulting mixture was stirred for 1 h at room temperature, then concentrated under reduced pressure. The residue was purified by trituration with PE/EA (1:1). The solid was removed by filtration and dried to give 6-bromoimidazo[1,5-a]pyridin-1-amine hydrochloride (500 mg, 95% yield) as a yellow solid.

LCMS (ES, m/z): [M+H]⁺: 212, 214

### Synthesis of 63-c: 1-[6-bromoimidazo[1,5-a]pyridin-1-yl]-1,2,3,4-tetrazole

A solution of 6-bromoimidazo[1,5-a]pyridin-1-amine (1 g, 4.8 mmol, 1 equiv), triethyl orthoformate (1.75 g, 12 mmol, 2.5 equiv) and sodium azide (770 mg, 12 mmol, 2.5 equiv) in CH₃COOH (10 mL) was stirred overnight at 90° C. under nitrogen atmosphere. The mixture was allowed to cool and was concentrated under reduced pressure. The resulting mixture was diluted with water (10 mL), then extracted with EtOAc (3×30 mL). The combined organics were washed with water and brine, dried over anhydrous Na₂SO₄ and concentrated under reduced pressure. The residue was purified by silica gel column chromatography, eluting with EtOAc/PE (0-100%) to afford 1-[6-bromoimidazo[1,5-a]pyridin-1-yl]-1,2,3,4-tetrazole (520 mg, 42% yield) as a yellow solid.

LCMS (ES, m/z): [M+H]⁺: 265, 266

### Synthesis of 63-d: 2,4-difluoro-3-[1-(1,2,3,4-tetrazol-1-yl)imidazo[1,5-a]pyridin-6-yl]aniline

A solution of 1-[6-bromoimidazo[1,5-a]pyridin-1-yl]-1,2,3,4-tetrazole (520 mg, 2 mmol, 1 equiv), 2,4-difluoro-3-(4,4,5,5-tetramethyl-1,3,2-dioxaborolan-2-yl)aniline (750 mg, 3 mmol, 1.5 equiv), Pd(dtbpf)Cl₂ (128 mg, 0.2 mmol, 0.1 equiv) and K₂CO₃ (813 mg, 5.9 mmol, 3 equiv) in dioxane (4.5 mL) and H₂O (0.5 mL) was stirred overnight at 80° C. under nitrogen atmosphere. The mixture was allowed to cool to room temperature and was diluted with water (10 mL). The resulting mixture was extracted with EtOAc (3×30 mL), and the combined organics were washed with water and brine, dried over anhydrous Na₂SO₄ and concentrated under reduced pressure. The residue was purified by silica gel column chromatography, eluting with EtOAc/PE (0-100%) to afford 2,4-difluoro-3-[1-(1,2,3,4-tetrazol-1-yl)imidazo[1,5-a]pyridin-6-yl]aniline (400 mg, 65% yield) as a yellow solid.

LCMS (ES, m/z): [M+H]⁺: 314

### Synthesis of Compound 63: 5-chloro-N-[2,4-difluoro-3-[1-(1,2,3,4-tetrazol-1-yl)imidazo[1,5-a]pyridin-6-yl]phenyl]-2-methoxypyridine-3-sulfonamide

To a stirred solution of 2,4-difluoro-3-[1-(1,2,3,4-tetrazol-1-yl)imidazo[1,5-a]pyridin-6-yl]aniline (50 mg, 0.16 mmol, 1 equiv) and pyridine (50 mg, 0.64 mmol, 4 equiv) in DCM (0.5 mL) was added 5-chloro-2-methoxypyridine-3-sulfonyl chloride (58 mg, 0.24 mmol, 1.5 equiv) dropwise at room temperature. The resulting mixture was stirred for 2 h, then diluted with water (5 mL). The resulting mixture was extracted with EtOAc (3×20 mL), and the combined organics were washed with water and brine, dried over anhydrous Na₂SO₄ and concentrated under reduced pressure. The crude product was purified by Prep-HPLC with the following conditions: Column, Sunfire Prep C18 OBD, 50*250 mm 5 μm 10 nm; mobile phase: 25-60% MeCN/0.1% aqueous formic acid; Detector, uv; to give 5-chloro-N-[2,4-difluoro-3-[1-(1,2,3,4-tetrazol-1-yl)imidazo[1,5-a]pyridin-6-yl]phenyl]-2-methoxypyridine-3-sulfonamide (17 mg, 20% yield) as a white solid.

LCMS (ES, m/z): [M+H]⁺: 519.

¹H NMR (300 MHz, DMSO-d₆) δ 10.48 (s, 1H), 10.05 (s, 1H), 8.66 (m, 2H), 8.50 (d, J=2.6 Hz, 1H), 8.09 (d, J=2.6 Hz, 1H), 7.91 (d, J=9.5 Hz, 1H), 7.39 (td, J=8.9, 5.9 Hz, 1H), 7.31-7.19 (m, 1H), 7.03 (dd, J=9.5, 1.5 Hz, 1H), 3.92 (s, 3H).

### Example 79: Synthesis of (R)—N-(3-(3-(1H-imidazol-2-yl)-5,6,7,8-tetrahydroimidazo[1,5-a]pyridin-7-yl)-2,4-difluorophenyl)-5-chloro-2-methoxypyridine-3-sulfonamide & (S)—N-(3-(3-(1H-imidazol-2-yl)-5,6,7,8-tetrahydroimidazo[1,5-a]pyridin-7-yl)-2,4-difluorophenyl)-5-chloro-2-methoxypyridine-3-sulfonamide (Compounds 64-1 & 64-2)

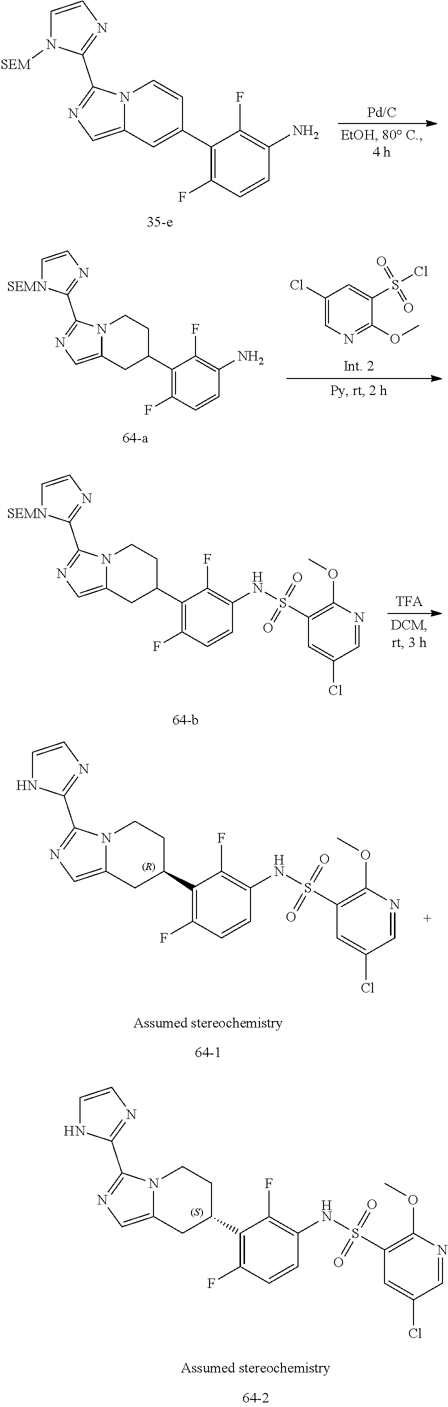

### Synthesis of 64-a: 2,4-difluoro-3-[3-(1-[[2-(trimethylsilyl)ethoxy]methyl]imidazol-2-yl)-5H,6H,7H,8H-imidazo[1,5-a]pyridin-7-yl]aniline

To a stirred solution of 2,4-difluoro-3-[3-(1-[[2-(trimethylsilyl)ethoxy]methyl]imidazol-2-yl)imidazo[1,5-a]pyridin-7-yl]aniline (500 mg, 1.1 mmol, 1 equiv) in EtOH (20 mL) was added Pd/C (120 mg, 10%). The resulting mixture was hydrogenated under 20 atm of hydrogen for 6 h at 80° C. After cooling, the mixture was filtered through celite and the filtrate concentrated under reduced pressure. The residue was purified by column chromatography over silica gel (eluent: PE:EA=1:1) to afford 2,4-difluoro-3-[3-(1-[[2-(trimethylsilyl)ethoxy]methyl]imidazol-2-yl)-5H,6H,7H,8H-imidazo[1,5-a]pyridin-7-yl]aniline (480 mg, 95% yield) as a brown solid.

LCMS (ES, m/z): [M+H]+: 446

### Synthesis of 64-b: 5-chloro-N-[2,4-difluoro-3-[3-(1-[[2-(trimethylsilyl)ethoxy]methyl]imidazol-2-yl)-5H,6H,7H,8H-imidazo[1,5-a]pyridin-7-yl]phenyl]-2-methoxypyridine-3-sulfonamide

To a stirred solution of 2,4-difluoro-3-[3-(1-[[2-(trimethylsilyl)ethoxy]methyl]imidazol-2-yl)-5H,6H,7H,8H-imidazo[1,5-a]pyridin-7-yl]aniline (480 mg, 1 mmol, 1 equiv) and pyridine (255 mg, 3.2 mmol, 3 equiv) in DCM (10 mL) were added 5-chloro-2-methoxypyridine-3-sulfonyl chloride (391 mg, 1.6 mmol, 1.5 equiv) in portions at room temperature. The resulting solution was stirred for 3 h then concentrated under pressure. The residue was purified by column chromatography over silica gel (eluent: PE:EA=5:1) to afford 5-chloro-N-[2,4-difluoro-3-[3-(1-[[2-(trimethylsilyl)ethoxy]methyl]imidazol-2-yl)-5H,6H,7H,8H-imidazo[1,5-a]pyridin-7-yl]phenyl]-2-methoxypyridine-3-sulfonamide (470 mg, 67% yield) as a yellow solid.

LCMS (ES, m/z): [M+H]⁺: 651 Synthesis of Compounds 64-1 & 64-2: Assumed (R)—N-(3-(3-(1H-imidazol-2-yl)-5,6,7,8-tetrahydroimidazo[1,5-a]pyridin-7-yl)-2,4-difluorophenyl)-5-chloro-2-methoxypyridine-3-sulfonamide and Assumed (S)—N-(3-(3-(1H-imidazol-2-yl)-5,6,7,8-tetrahydroimidazo[1,5-a]pyridin-7-yl)-2,4-difluorophenyl)-5-chloro-2-methoxypyridine-3-sulfonamide

To a stirred solution of 5-chloro-N-[2,4-difluoro-3-[3-(1-[[2-(trimethylsilyl)ethoxy]methyl]imidazol-2-yl)-5H,6H,7H,8H-imidazo[1,5-a]pyridin-7-yl]phenyl]-2-methoxypyridine-3-sulfonamide (450 mg, 0.7 mmol, 1 equiv) in DCM (5 mL) was added TFA (1 mL) dropwise at room temperature. The resulting mixture was stirred for 1 h at room temperature, then concentrated under reduced pressure. The residue was purified by prep-HPLC with following conditions: Column, welch Vltimate XB-C18, 50×250 mm, m; Mobile Phase: 30-50% MeCN/0.1% aqueous formic acid; Detector, 220 nm; and chiral prep-HPLC with the following conditions: Column, YMC, SC, 250×30 mm, 5 μm; Mobile Phase 50% EtOH/Hexane; to afford (R)—N-(3-(3-(1H-imidazol-2-yl)-5,6,7,8-tetrahydroimidazo[1,5-a]pyridin-7-yl)-2,4-difluorophenyl)-5-chloro-2-methoxypyridine-3-sulfonamide (70 mg, 19% yield, stereochemistry assumed, RT=10 min) as a white solid and (S)—N-(3-(3-(1H-imidazol-2-yl)-5,6,7,8-tetrahydroimidazo[1,5-a]pyridin-7-yl)-2,4-difluorophenyl)-5-chloro-2-methoxypyridine-3-sulfonamide (80 mg, 22% yield, stereochemistry assumed, RT=12 min) as a white solid.

Assumed-(R)—N-(3-(3-(1H-imidazol-2-yl)-5,6,7,8-tetrahydroimidazo[1,5-a]pyridin-7-yl)-2,4-difluorophenyl)-5-chloro-2-methoxypyridine-3-sulfonamide

LCMS (ES, m/z): [M+H]⁺: 521

¹H NMR (300 MHz, DMSO-d₆) δ 12.57 (s, 1H), 10.29 (s, 1H), 8.50 (d, J=2.6 Hz, 1H), 8.00 (d, J=2.5 Hz, 1H), 7.31-6.95 (m, 4H), 6.77 (s, 1H), 5.10-4.89 (m, 1H), 4.10 (td, J=12.9, 4.5 Hz, 1H), 3.93 (s, 3H), 3.47-3.35 (m, 1H), 3.06-2.74 (m, 2H), 2.33-2.23 (m, 1H), 2.07 (d, J=13.3 Hz, 1H).

Assumed-(S)—N-(3-(3-(1H-imidazol-2-yl)-5,6,7,8-tetrahydroimidazo[1,5-a]pyridin-7-yl)-2,4-difluorophenyl)-5-chloro-2-methoxypyridine-3-sulfonamide

LCMS (ES, m/z): [M+H]⁺: 521

¹H NMR (300 MHz, DMSO-d₆) δ 12.56 (s, 1H), 10.29 (s, 1H), 8.51 (d, J=2.6 Hz, 1H), 8.00 (d, J=2.6 Hz, 1H), 7.30-6.95 (m, 4H), 6.77 (s, 1H), 4.97 (d, J=14.3 Hz, 1H), 4.10 (t, J=11.1 Hz, 1H), 3.93 (s, 3H), 3.39 (s, 1H), 3.07-2.77 (m, 2H), 2.27 (q, J=1.9 Hz, 1H), 2.07 (d, J=12.8 Hz, 1H).

### Example 80: Synthesis of 5-chloro-N-[2,4-difluoro-3-[1-(4H-1,2,4-triazol-3-yl)imidazo[1,5-a]pyridin-6-yl]phenyl]-2-methylpyridine-3-sulfonamide (Compound 65)

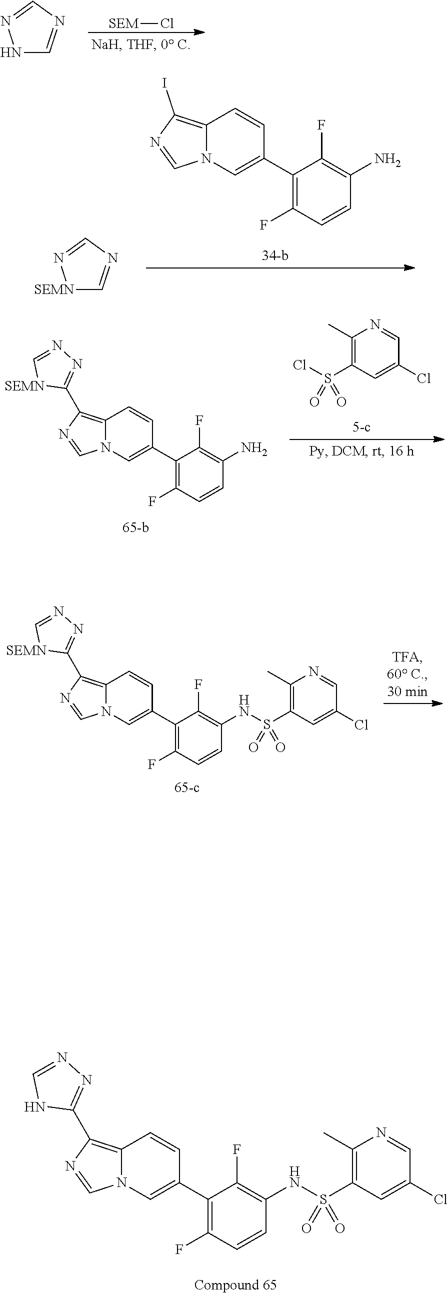

### Synthesis of 65-a: 1-[[2-(trimethylsilyl)ethoxy]methyl]-1,2,4-triazole

Into a 100 mL 3-necked round-bottom flask were added 1,2,4-triazole (2 g, 29 mmol, 1 equiv) and DMF (20 mL). To the above mixture was added NaH (2.1 g, 88 mmol, 3 equiv) in portions at 0° C. The resulting mixture was stirred for 30 min at 0° C. and [2-(chloromethoxy)ethyl]trimethylsilane (5.3 g, 32 mmol, 1.1 equiv) was added dropwise. The resulting mixture was stirred for 1 h at 0° C., then quenched with water (100 mL). The resulting mixture was diluted further with water (100 mL) and extracted with EtOAc (3×100 mL). The combined organic layers were washed with brine (3×50 mL), dried over anhydrous Na₂SO₄ and concentrated under reduced pressure. The residue was purified by silica gel column chromatography, eluting with PE/EtOAc (10:1) to afford 1-[[2-(trimethylsilyl)ethoxy]methyl]-1,2,4-triazole (1.5 g, 26% yield) as a colorless oil.

LCMS (ES, m/z): [M+H]⁺: 200.

### Synthesis of 65-b: 2,4-difluoro-3-[1-(4-[[2-(trimethylsilyl)ethoxy]methyl]-1,2,4-triazol-3-yl)imidazo[1,5-a]pyridin-6-yl]aniline

Into a 100 mL 3-necked round-bottom flask was added 4-[[2-(trimethylsilyl)ethoxy]methyl]-1,2,4-triazole (900 mg, 4.5 mmol, 1 equiv) in THE (20 mL). To this was added 2.5M n-BuLi (2.7 mL, 6.7 mmol, 1.5 equiv) dropwise at −78° C. The resulting mixture was stirred for 1 h at −78° C., then 1M ZnCl₂ in Et₂O (4.5 mL, 4.5 mmol, 1 equiv) was added. The resulting mixture was stirred from −78° C. to room temperature over 1 hr, then 2,4-difluoro-3-[1-iodoimidazo[1,5-a]pyridin-6-yl]aniline (552 mg, 1.5 mmol, 0.33 equiv) and Pd(PPh₃)₄ (521 mg, 0.45 mmol, 0.1 equiv) were added. The reaction was stirred for 1 h at 60° C., then cooled and diluted with water (100 mL). This mixture was extracted with EtOAc (3×100 mL), and the combined organics were washed with brine (3×50 mL), dried over anhydrous Na₂SO₄ and concentrated under reduced pressure. The residue was purified by silica gel column chromatography, eluting with PE/EtOAc (3:2) to afford 2,4-difluoro-3-[1-(4-[[2-(trimethylsilyl)ethoxy]methyl]-1,2,4-triazol-3-yl)imidazo[1,5-a]pyridin-6-yl]aniline (700 mg, 30% yield) as a light brown solid.

LCMS (ES, m/z): [M+H]⁺: 443

### Synthesis of 65-c: 5-chloro-N-[2,4-difluoro-3-[1-(4-[[2-(trimethylsilyl)ethoxy]methyl]-1,2,4-triazol-3-yl) imidazo[1,5-a]pyridin-6-yl]phenyl]-2-methylpyridine-3-sulfonamide

Into an 8 mL vial were added 2,4-difluoro-3-[1-(4-[[2-(trimethylsilyl)ethoxy]methyl]-1,2,4-triazol-3-yl)imidazo[1,5-a]pyridin-6-yl]aniline (150 mg, 0.3 mmol, 1 equiv), 5-chloro-2-methylpyridine-3-sulfonyl chloride (114 mg, 0.5 mmol, 1.5 equiv), DCM (5 mL) and pyridine (134 mg, 1.7 mmol, 5 equiv). The resulting mixture was stirred overnight at room temperature, then concentrated under vacuum. The residue was purified by silica gel column chromatography, eluting with PE/EtOAc (1:1) to afford 5-chloro-N-[2,4-difluoro-3-[1-(4-[[2-(trimethylsilyl)ethoxy]methyl]-1,2,4-triazol-3-yl)imidazo[1,5-a]pyridin-6-yl]phenyl]-2-methylpyridine-3-sulfonamide (130 mg, 61% yield) as a light brown solid.

LCMS (ES, m/z): [M+H]⁺: 632.

### Synthesis of Compound 65: 5-chloro-N-[2,4-difluoro-3-[1-(4H-1,2,4-triazol-3-yl)imidazo[1,5-a]pyridin-6-yl]phenyl]-2-methylpyridine-3-sulfonamide

Into an 8 mL vial were added 5-chloro-N-[2,4-difluoro-3-[1-(4-[[2-(trimethylsilyl)ethoxy]methyl]-1,2,4-triazol-3-yl)imidazo[1,5-a]pyridin-6-yl]phenyl]-2-methylpyridine-3-sulfonamide (107 mg, 0.17 mmol, 1 equiv) and TFA (2 mL). The resulting mixture was stirred for 30 min at 60° C., then was concentrated under vacuum. The crude product was purified by Prep-HPLC with the following conditions: Column: Atlantis HILIC OBD, 19*150 mm*5 μm; Mobile Phase: 20-50% MeCN/0.1% aqueous formic acid; Flow rate: 90 mL/min; Detector 220 nm; to afford 5-chloro-N-[2,4-difluoro-3-[1-(4H-1,2,4-triazol-3-yl)imidazo[1,5-a]pyridin-6-yl]phenyl]-2-methylpyridine-3-sulfonamide (22 mg, 25% yield) as a white solid.

LCMS (ES, m/z): [M+H]⁺: 502.

¹H NMR (300 MHz, Methanol-d₄) δ 8.65 (d, J=2.4 Hz, 1H), 8.47 (s, 1H), 8.43 (d, J=1.4 Hz, 1H), 8.30 (s, 1H), 8.22 (d, J=9.5 Hz, 1H), 8.16 (d, J=2.4 Hz, 1H), 7.54 (td, J=8.9, 5.7 Hz, 1H), 7.17 (td, J=9.2, 1.9 Hz, 1H), 6.95 (dd, J=9.4, 1.5 Hz, 1H), 2.84 (s, 3H).

### Example 81: Synthesis of N-[3-[1-(1H-1,3-benzodiazol-2-yl)imidazo[1,5-a]pyridin-6-yl]-2,4-difluorophenyl]-5-cyano-2-methoxypyridine-3-sulfonamide (Compound 66)

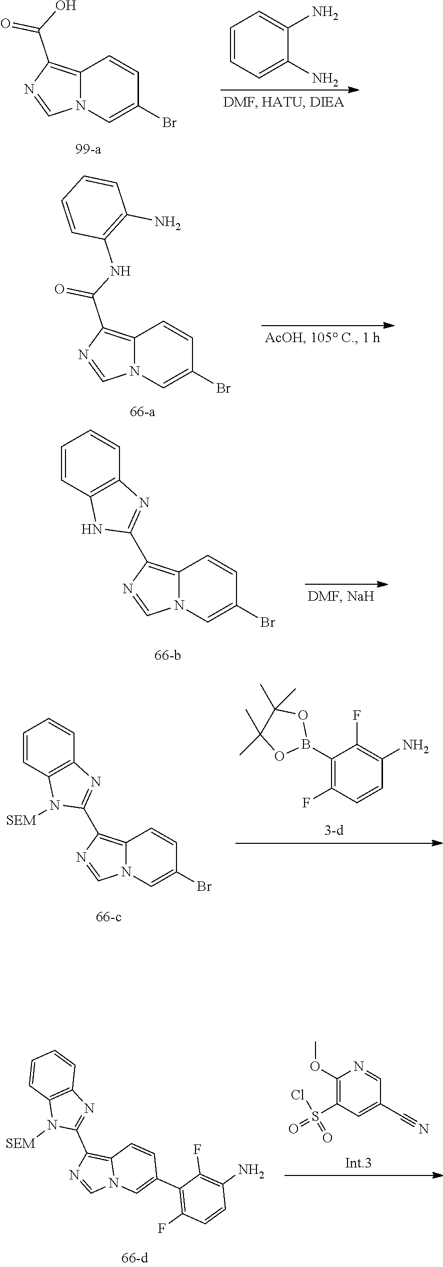

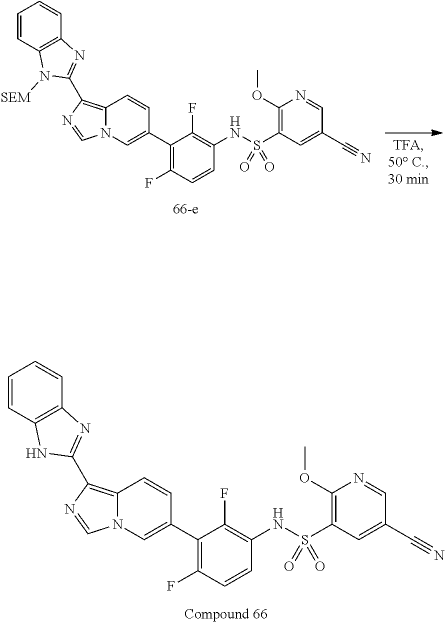 [SMILES: COc1ncc(C#N)cc1S(=O)(=O)Nc1ccc(F)c(-c2ccc3c(-c4nc5ccccc5n4COCC[Si](C)(C)C)ncn3c2)c1F ; COc1ncc(C#N)cc1S(=O)(=O)Nc1ccc(F)c(-c2ccc3c(-c4nc5ccccc5[nH]4)ncn3c2)c1F]

### Synthesis of 66-a: N-(2-aminophenyl)-6-bromoimidazo[1,5-a]pyridine-1-carboxamide

Into a 100 mL round-bottom flask were added 6-bromoimidazo[1,5-a]pyridine-1-carboxylic acid (1 g, 4.2 mmol, 1 equiv) and DMF (30 mL). To the stirred solution was added HATU (2.4 g, 6.2 mmol, 1.5 equiv) and DIEA (1.07 g, 8.3 mmol, 2 equiv). The resulting mixture was stirred for 10 min when o-phenylenediamine (0.54 g, 5 mmol, 1.2 equiv) was added. The resulting mixture was stirred overnight at room temperature, then was diluted with water (100 mL). The precipitated solids were collected by filtration and washed with water (3×10 mL) to afford N-(2-aminophenyl)-6-bromoimidazo[1,5-a]pyridine-1-carboxamide (900 mg, 66% yield) as a brown solid.

LCMS (ES, m/z): [M+H]⁺:331, 333.

### Synthesis of 66-b: 2-[6-bromoimidazo[1,5-a]pyridin-1-yl]-1H-1,3-benzodiazole

Into a 20 mL vial were added N-(2-aminophenyl)-6-bromoimidazo[1,5-a]pyridine-1-carboxamide (800 mg, 2.4 mmol, 1 equiv) and AcOH (8 mL). The resulting mixture was stirred for 1 h at 105° C., then was cooled and concentrated under reduced pressure. The mixture was basified to pH 8 with saturated NaHCO₃ (aq.) and extracted with EtOAc (3×30 mL). The combined organics were washed with brine (30 mL), dried over anhydrous Na₂SO₄ and concentrated under reduced pressure. The residue was purified by silica gel column chromatography, eluting with PE/EtOAc (1:1) to afford 2-[6-bromoimidazo[1,5-a]pyridin-1-yl]-1H-1,3-benzodiazole (720 mg, 95% yield) as a yellow solid.

LCMS (ES, m/z): [M+H]: 313, 315

### Synthesis of 66-c: 2-[6-bromoimidazo[1,5-a]pyridin-1-yl]-1-[[2-(trimethylsilyl)ethoxyl methyl]-1,3-benzodiazole

Into a 100 mL 3-necked round-bottom flask were added 2-[6-bromoimidazo[1,5-a]pyridin-1-yl]-1H-1,3-benzodiazole (660 mg, 2.1 mmol, 1 equiv) and DMF (15 mL). To the stirred solution was added 60% NaH in oil (65 mg, 2.7 mmol, 1.3 equiv) at 0-5° C. and this mixture was stirred for 10 min at room temperature. SEM-Cl (456 mg, 2.74 mmol, 1.3 equiv) was added dropwise, and the reaction was stirred for an additional 30 min at room temperature, before being quenched with water/ice (60 mL). The resulting mixture was extracted with EtOAc (3×20 mL), and the combined organics were washed with brine (20 mL), dried over anhydrous Na₂SO₄ and concentrated under reduced pressure. The residue was purified by silica gel column chromatography, eluting with PE/EtOAc (5:1) to afford 2-[6-bromoimidazo[1,5-a]pyridin-1-yl]-1-[[2-(trimethylsilyl)ethoxy]methyl]-1,3-benzodiazole (750 mg, 80% yield) as a yellow solid.

LCMS (ES, m/z): [M+H]: 443, 445

### Synthesis of 66-d: 2,4-difluoro-3-[1-(1-[[2-(trimethylsilyl)ethoxy]methyl]-1,3-benzodiazol-2-yl)imidazo[1,5-a]pyridin-6-yl]aniline

Into a 20 mL vial were added 2-[6-bromoimidazo[1,5-a]pyridin-1-yl]-1-[[2-(trimethylsilyl)ethoxy]methyl]-1,3-benzodiazole (700 mg, 1.6 mmol, 1 equiv), dioxane (14 mL) and H₂O (1.4 mL). To the stirred solution were added 2,4-difluoro-3-(4,4,5,5-tetramethyl-1,3,2-dioxaborolan-2-yl)aniline (604 mg, 2.4 mmol, 1.5 equiv), K₂CO₃ (654 mg, 4.7 mmol, 3 equiv) and Pd(dtbpf)Cl₂ (103 mg, 0.16 mmol, 0.1 equiv). The reaction was stirred for 1 h at 80° C., then cooled and diluted with water (30 mL). The resulting mixture was extracted with EtOAc (3×20 mL), and the combined organic layers were washed with brine (10 mL), dried over anhydrous Na₂SO₄ and concentrated under reduced pressure. The residue was purified by silica gel column chromatography, eluting with PE/EtOAc (3:1) to afford 2,4-difluoro-3-[1-(1-[[2-(trimethylsilyl)ethoxy]methyl]-1,3-benzodiazol-2-yl)imidazo[1,5-a]pyridin-6-yl]aniline (500 mg, 64% yield) as a light yellow solid.

LCMS (ES, m/z): [M+H]⁺: 492.

### Synthesis of 66-e: 5-cyano-N-[2,4-difluoro-3-[1-(1-[[2-(trimethylsilyl)ethoxy]methyl]-1,3-benzodiazol-2-yl)imidazo[1,5-a]pyridin-6-yl]phenyl]-2-methoxypyridine-3-sulfonamide

Into a 20 mL vial were added 2,4-difluoro-3-[1-(1-[[2-(trimethylsilyl)ethoxy]methyl]-1,3-benzodiazol-2-yl)imidazo[1,5-a]pyridin-6-yl]aniline (130 mg, 0.26 mmol, 1 equiv) and DCM (4 mL). To this stirred solution were added pyridine (104 mg, 1.3 mmol, 5 equiv) and 5-cyano-2-methoxypyridine-3-sulfonyl chloride (92 mg, 0.39 mmol, 1.5 equiv) and the reaction was stirred for 1 h. The resulting mixture was concentrated under reduced pressure and the residue purified by silica gel column chromatography, eluting with PE/THF (5:1) to afford 5-cyano-N-[2,4-difluoro-3-[1-(1-[[2-(trimethylsilyl)ethoxy]methyl]-1,3-benzodiazol-2-yl)imidazo[1,5-a]pyridin-6-yl]phenyl]-2-methoxypyridine-3-sulfonamide (150 mg, 77% yield) as a yellow oil.

LCMS (ES, m/z): [M+H]: 688.

### Synthesis of Compound 66: of N-[3-[1-(1H-1,3-benzodiazol-2-yl)imidazo[1,5-a]pyridin-6-yl]-2,4-difluorophenyl]-5-cyano-2-methoxypyridine-3-sulfonamide

Into a 2 mL vial were added 5-cyano-N-[2,4-difluoro-3-[1-(1-[[2-(trimethylsilyl)ethoxy]methyl]-1,3-benzodiazol-2-yl)imidazo[1,5-a]pyridin-6-yl]phenyl]-2-methoxypyridine-3-sulfonamide (130 mg, 0.19 mmol, 1 equiv) and TFA (2 mL). The resulting mixture was stirred for 30 min at 55° C. under nitrogen atmosphere, then concentrated under reduced pressure. The mixture was basified to pH 8 with ammonia, and the crude product was purified by Prep-HPLC using the following conditions: Column, welch Vltimate XB-C18, 50×250 mm, 10 μm; Flowrate: 90 mL/min, Mobile Phase: 16-51% MeCN/0.05% aqueous ammonia; to afford N-[3-[1-(1H-1,3-benzodiazol-2-yl)imidazo[1,5-a]pyridin-6-yl]-2,4-difluorophenyl]-5-cyano-2-methoxypyridine-3-sulfonamide (50 mg, 48% yield) as a white solid.

LCMS (ES, m/z): [M+H]⁺: 558.

¹H NMR (300 MHz, DMSO-d₆) δ 12.74 (s, 1H), 10.56 (s, 1H), 8.94 (d, J=2.2 Hz, 1H), 8.65 (d, J=3.6 Hz, 2H), 8.54-8.41 (m, 2H), 7.62 (s, 1H), 7.47 (s, 1H), 7.40 (td, J=8.9, 5.9 Hz, 1H), 7.32-7.21 (m, 1H), 7.21-7.10 (m, 2H), 7.03 (d, J=9.6 Hz, 1H), 4.03 (s, 3H).

### Example 82: Synthesis of (R)-6-(2,6-difluoro-3-((5-fluoro-2-methoxypyridine)-3-sulfonamido)phenyl)-N-methyl-5,6,7,8-tetrahydroimidazo[1,5-a]pyridine-1-carboxamide and (S)-6-(2,6-difluoro-3-((5-fluoro-2-methoxypyridine)-3-sulfonamido)phenyl)-N-methyl-5,6,7,8-tetrahydroimidazo[1,5-a]pyridine-1-carboxamide (Compounds 67-1 & 67-2)

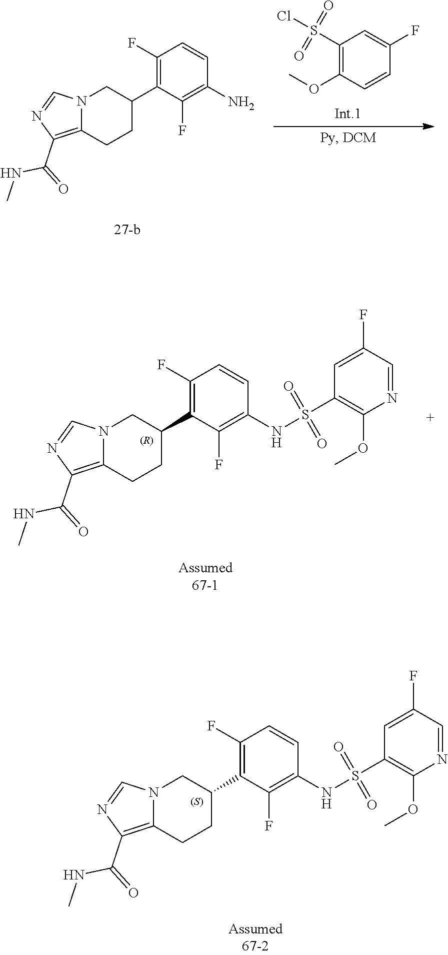

### Synthesis of Compounds 67-1 & 67-2: (R)-6-(2,6-difluoro-3-((5-fluoro-2-methoxypyridine)-3-sulfonamido)phenyl)-N-methyl-5,6,7,8-tetrahydroimidazo[1,5-a]pyridine-1-carboxamide and (S)-6-(2,6-difluoro-3-((5-fluoro-2-methoxypyridine)-3-sulfonamido)phenyl)-N-methyl-5,6,7,8-tetrahydroimidazo[1,5-a]pyridine-1-carboxamide

To a stirred solution of 6-(3-amino-2,6-difluorophenyl)-N-methyl-5H,6H,7H,8H-imidazo[1,5-a]pyridine-1-carboxamide (200 mg, 0.65 mmol, 1 equiv) in pyridine (5 mL) were added a solution of 5-fluoro-2-methoxypyridine-3-sulfonyl chloride (221 mg, 0.98 mmol, 1.5 equiv) in DCM (1 mL) dropwise at room temperature. The solution was stirred for 1 h at room temperature, then quenched with water (20 mL). The resulting mixture was extracted with EA (3×20 mL), and the combined organics were washed with brine (2×10 mL), dried over anhydrous Na₂SO₄ and concentrated under reduced pressure. The residue was purified by prep-HPLC with the following conditions: Column, Atlantis HILIC OBD, 19*150 mm*5 μm; Mobile Phase 20-50% MeCN/0.1% aqueous formic acid; and chiral-prep-HPLC with the following conditions: Column, CHIRALPAK SB, 250*30 mm, 5 m, Mobile Phase 10% EtOH/Hexane: DCM (5:1); to afford (6R)-6-[2,6-difluoro-3-(5-fluoro-2-methoxypyridine-3-sulfonamido)phenyl]-N-methyl-5H,6H,7H,8H-imidazo[1,5-a]pyridine-1-carboxamide (65 mg, 20% yield, stereochemistry assumed) as a white solid and (6S)-6-[2,6-difluoro-3-(5-fluoro-2-methoxypyridine-3-sulfonamido)phenyl]-N-methyl-5H,6H,7H,8H-imidazo[1,5-a]pyridine-1-carboxamide (59 mg, 18% yield, stereochemistry assumed) as a white solid.

Assumed-(R)-6-(2,6-difluoro-3-((5-fluoro-2-methoxypyridine)-3-sulfonamido)phenyl)-N-methyl-5,6,7,8-tetrahydroimidazo[1,5-a]pyridine-1-carboxamide

LCMS (ES, m/z): [M+H]⁺: 496

¹H NMR (300 MHz, DMSO-d₆) δ 10.30 (s, 1H), 8.48 (d, J=3.0 Hz, 1H), 7.97 (dd, J=7.4, 3.0 Hz, 1H), 7.74 (d, J=5.0 Hz, 1H), 7.54 (s, 1H), 7.32-7.18 (m, 1H), 7.09 (t, J=9.5 Hz, 1H), 4.26 (dd, J=12.4, 5.3 Hz, 1H), 4.04-3.91 (m, 1H), 3.91 (s, 3H), 3.49 (s, 1H), 2.87 (ddd, J=17.3, 11.6, 6.2 Hz, 1H), 2.71 (d, J=4.8 Hz, 3H), 2.07 (s, 1H), 1.96 (s, 1H).

Assumed-(S)-6-(2,6-difluoro-3-((5-fluoro-2-methoxypyridine)-3-sulfonamido)phenyl)-N-methyl-5,6,7,8-tetrahydroimidazo[1,5-a]pyridine-1-carboxamide

LCMS (ES, m/z): [M+H]⁺: 496

¹H NMR (300 MHz, DMSO-d₆) δ 10.30 (s, 1H), 8.47 (d, J=3.0 Hz, 1H), 7.97 (dd, J=7.3, 3.0 Hz, 1H), 7.74 (d, J=5.0 Hz, 1H), 7.54 (s, 1H), 7.25 (td, J=8.8, 5.7 Hz, 1H), 7.09 (t, J=9.7 Hz, 1H), 4.26 (dd, J=12.2, 5.2 Hz, 1H), 4.04-3.91 (m, 1H), 3.91 (s, 3H), 3.48 (s, 1H), 2.87 (ddd, J=18.1, 11.8, 6.5 Hz, 1H), 2.75-2.67 (m, 3H), 2.05 (s, 1H), 1.96 (s, 1H).

### Example 83: Synthesis of (R)-6-(3-((5-cyano-2-methoxypyridine)-3-sulfonamido)-2,6-difluorophenyl)-N-methyl-5,6,7,8-tetrahydroimidazo[1,5-a]pyridine-1-carboxamide and (S)-6-(3-((5-cyano-2-methoxypyridine)-3-sulfonamido)-2,6-difluorophenyl)-N-methyl-5,6,7,8-tetrahydroimidazo[1,5-a]pyridine-1-carboxamide (Compounds 68-1 & 68-2)

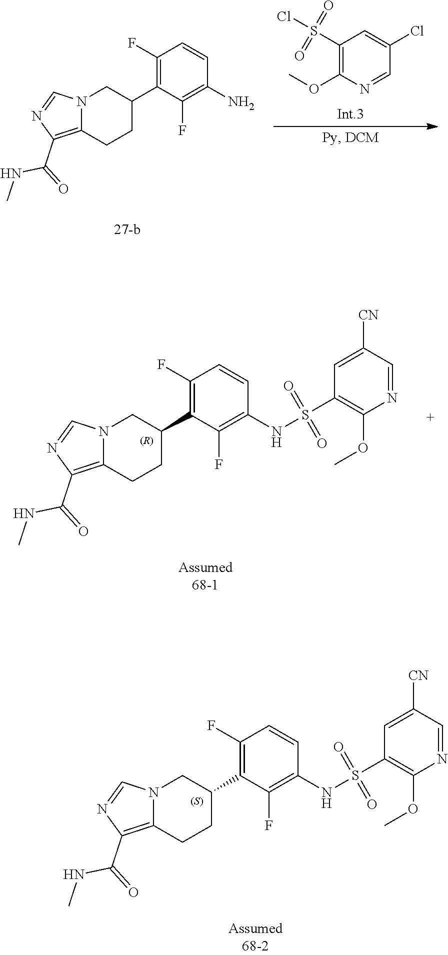

### Synthesis of Compounds 68-1 & 68-2: (R)-6-(3-((5-cyano-2-methoxypyridine)-3-sulfonamido)-2,6-difluorophenyl)-N-methyl-5,6,7,8-tetrahydroimidazo[1,5-a]pyridine-1-carboxamide and (S)-6-(3-((5-cyano-2-methoxypyridine)-3-sulfonamido)-2,6-difluorophenyl)-N-methyl-5,6,7,8-tetrahydroimidazo[1,5-a]pyridine-1-carboxamide

To a stirred solution of 6-(3-amino-2,6-difluorophenyl)-N-methyl-5H,6H,7H,8H-imidazo[1,5-a]pyridine-1-carboxamide (200 mg, 0.65 mmol, 1 equiv) in pyridine (5 mL) was added a solution of 5-cyano-2-methoxypyridine-3-sulfonyl chloride (227 mg, 0.98 mmol, 1.5 equiv) in DCM (1 mL) dropwise at room temperature. The resulting solution was stirred for 1 h at room temperature, then quenched with water (20 mL). The resulting mixture was extracted with EA (3×20 mL), and the combined organics were washed with brine (2×10 mL), dried over anhydrous Na₂SO₄, then concentrated under reduced pressure. The residue was purified by prep-HPLC with the following conditions: Column: Atlantis HILIC OBD, 19*150 mm*5 μm; Mobile Phase 20-50% MeCN/0.1% aqueous formic acid; Flow rate: 90 mL/min; Detector 220 nm; and chiral-prep-HPLC with the following conditions: Column: CHIRALPAK IG, 250*30 mm, 5 m; Mobile Phase 30% EtOH/3:1 Hexane: DCM; to afford (6R)-6-[3-(5-cyano-2-methoxypyridine-3-sulfonamido)-2,6-difluorophenyl]-N-methyl-5H,6H,7H,8H-imidazo[1,5-a]pyridine-1-carboxamide (56 mg, 17% yield, stereochemistry randomly assigned) as a white solid and (6S)-6-[3-(5-cyano-2-methoxypyridine-3-sulfonamido)-2,6-difluorophenyl]-N-methyl-5H,6H,7H,8H-imidazo[1,5-a]pyridine-1-carboxamide (48 mg, 15% yield, stereochemistry randomly assigned) as a white solid.

Assumed-(R)-6-(3-((5-cyano-2-methoxypyridine)-3-sulfonamido)-2,6-difluorophenyl)-N-methyl-5,6,7,8-tetrahydroimidazo[1,5-a]pyridine-1-carboxamide

LCMS (ES, m/z): [M+H]⁺: 503

¹H NMR (300 MHz, DMSO-d₆) δ 10.41 (s, 1H), 8.93 (d, J=2.2 Hz, 1H), 8.43 (d, J=2.2 Hz, 1H), 7.74 (d, J=4.9 Hz, 1H), 7.54 (s, 1H), 7.24 (dt, J=8.9, 4.4 Hz, 1H), 7.08 (t, J=9.5 Hz, 1H), 4.26 (dd, J=12.3, 5.3 Hz, 1H), 4.01 (s, 3H), 3.95 (d, J=12.0 Hz, 1H), 3.48 (s, 1H), 3.29 (s, 1H), 2.97-2.75 (m, 1H), 2.72 (t, J=4.1 Hz, 3H), 2.15-1.88 (m, 2H).

Assumed-(S)-6-(3-((5-cyano-2-methoxypyridine)-3-sulfonamido)-2,6-difluorophenyl)-N-methyl-5,6,7,8-tetrahydroimidazo[1,5-a]pyridine-1-carboxamide

LCMS (ES, m/z): [M+H]⁺: 503

¹H NMR (300 MHz, DMSO-d₆) δ 10.41 (s, 1H), 8.94 (d, J=2.2 Hz, 1H), 8.44 (d, J=2.3 Hz, 1H), 7.74 (d, J=4.8 Hz, 1H), 7.54 (s, 1H), 7.26 (d, J=6.1 Hz, 1H), 7.11 (d, J=9.6 Hz, 1H), 4.32-4.15 (m, 1H), 4.01 (s, 3H), 3.95 (d, J=11.9 Hz, 1H), 3.48 (s, 1H), 3.31 (s, 1H), 2.97-2.78 (m, 1H), 2.72 (t, J=4.1 Hz, 3H), 2.14-1.89 (m, 2H).

### Example 84: Synthesis of 5-cyano-N-[2,4-difluoro-3-[1-(1H-imidazol-2-yl)imidazo[1,5-a1 pyrazin-6-yl]phenyl]-2-methoxypyridine-3-sulfonamide (Compound 69)

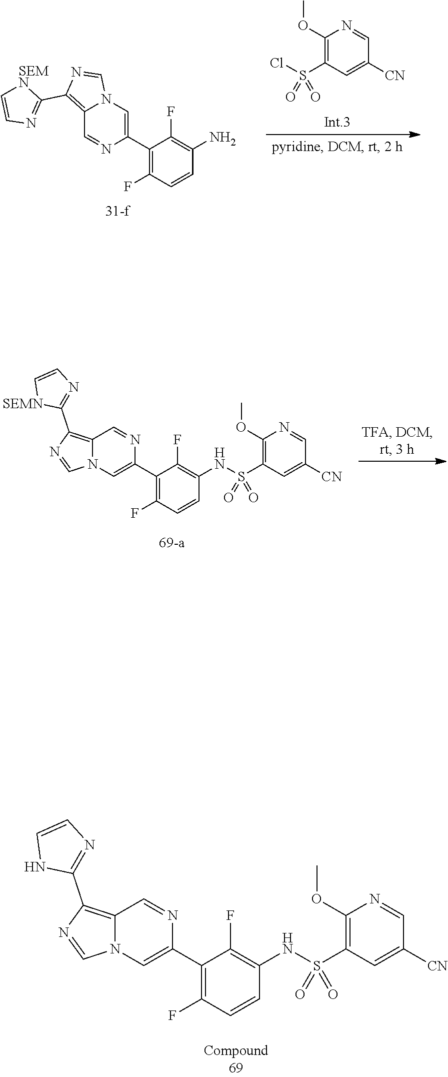 [SMILES: C[Si](C)(C)CCOCn1ccnc1-c1ncn2cc(-c3c(F)ccc(N)c3F)ncc12 ; COc1ncc(C#N)cc1S(=O)(=O)Cl ; COc1ncc(C#N)cc1S(=O)(=O)Nc1ccc(F)c(-c2cn3cnc(-c4nccn4COCC[Si](C)(C)C)c3cn2)c1F ; COc1ncc(C#N)cc1S(=O)(=O)Nc1ccc(F)c(-c2cn3cnc(-c4ncc[nH]4)c3cn2)c1F]

### Synthesis of 69-a: 5-cyano-N-[2,4-difluoro-3-[1-(1-[[2-(trimethylsilyl)ethoxy]methyl]imidazol-2-yl) imidazo[1,5-a]pyrazin-6-yl]phenyl]-2-methoxypyridine-3-sulfonamide

To a stirred solution of 2,4-difluoro-3-[1-(1-[[2-(trimethylsilyl)ethoxy]methyl]imidazol-2-yl)imidazo[1,5-a]pyrazin-6-yl]aniline (140 mg, 0.3 mmol, 1 equiv) in pyridine (3 mL) was added 5-cyano-2-methoxypyridine-3-sulfonyl chloride (95 mg, 0.4 mmol, 1.3 equiv) in portions at room temperature. The resulting mixture was stirred for 2 h at room temperature, then concentrated under reduced pressure. The residue was purified by Flash-Prep-HPLC with the following conditions: Column, WelFlash™ C18-I, Spherical C18 20-40 m, 120 g; mobile phase: 5-70% MeCN/0.1% aqueous formic acid; Detector, 220 nm; to give 5-cyano-N-[2,4-difluoro-3-[1-(1-[[2-(trimethylsilyl) ethoxy]methyl]imidazol-2-yl)imidazo[1,5-a]pyrazin-6-yl]phenyl]-2-methoxypyridine-3-sulfonamide (109 mg, 54% yield) as a yellow solid.

LCMS (ES, m/z): [M+H]⁺: 639

### Synthesis of Compound 69: 5-cyano-N-[2,4-difluoro-3-[1-(1H-imidazol-2-yl)imidazo[1,5-a]pyrazin-6-yl]phenyl]-2-methoxypyridine-3-sulfonamide

Into an 8 mL vial, was placed 5-cyano-N-[2,4-difluoro-3-[1-(1-[[2-(trimethylsilyl)ethoxy]methyl]imidazol-2-yl) imidazo[1,5-a]pyrazin-6-yl]phenyl]-2-methoxypyridine-3-sulfonamide (100 mg, 0.2 mmol, 1 equiv), DCM (3 mL) and TFA (1 mL). The resulting solution was stirred for 3 h at 25° C., then concentrated, and the residue diluted with 3 mL of MeOH. The pH was adjusted to 8 with NH₃ (7 M in MeOH), and this was purified by prep-HPLC with the following conditions: Column, welch Vltimate XB-C18, 50×250 mm, 10 μm; Mobile Phase: 5-45% MeCN/0.1% aqueous formic acid; Detector, 220 nm; to give 5-cyano-N-[2,4-difluoro-3-[1-(1H-imidazol-2-yl)imidazo[1,5-a]pyrazin-6-yl]phenyl]-2-methoxypyridine-3-sulfonamide (62 mg, 78% yield) as a yellow solid.

LCMS (ES, m/z): [M+H]⁺: 509

¹H NMR (300 MHz, DMSO-d₆) δ 9.55 (d, J=1.6 Hz, 1H), 8.93 (d, J=2.2 Hz, 1H), 8.66 (s, 1H), 8.61-8.39 (m, 2H), 7.42 (td, J=8.8, 5.8 Hz, 1H), 7.31-6.97 (m, 3H), 4.03 (s, 3H).

### Example 85: Synthesis of N-[2,4-difluoro-3-[1-(4H-1,2,4-triazol-3-yl)imidazo[1,5-a]pyridin-6-yl]phenyl]-5-fluoro-2-methoxypyridine-3-sulfonamide (Compound 70)

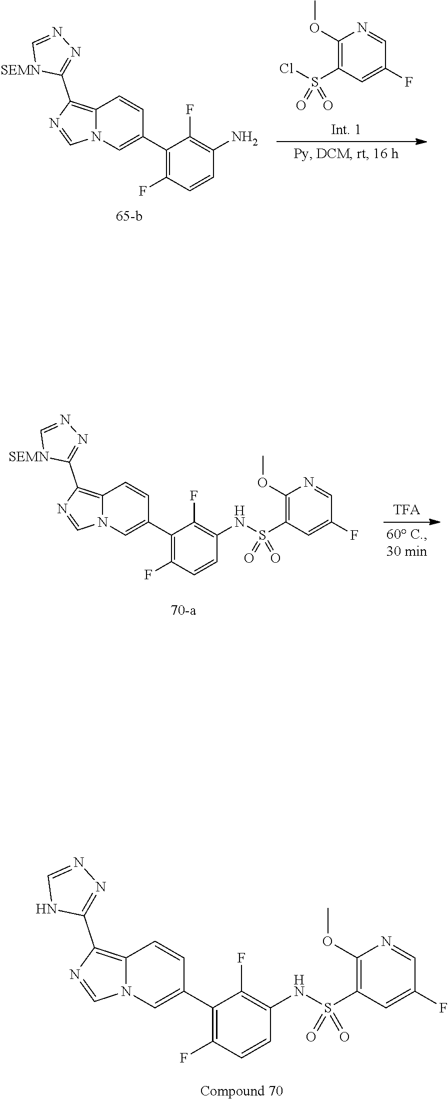 [SMILES: C[Si](C)(C)CCOCn1cnnc1-c1ncn2cc(-c3c(F)ccc(N)c3F)ccc12 ; COc1ncc(F)cc1S(=O)(=O)Cl ; COc1ncc(F)cc1S(=O)(=O)Nc1ccc(F)c(-c2ccc3c(-c4nncn4COCC[Si](C)(C)C)ncn3c2)c1F ; COc1ncc(F)cc1S(=O)(=O)Nc1ccc(F)c(-c2ccc3c(-c4nnc[nH]4)ncn3c2)c1F]

### Synthesis of 70-a: N-[2,4-difluoro-3-[1-(4-[[2-(trimethylsilyl)ethoxy]methyl]-1,2,4-triazol-3-yl)imidazo[1,5-a]pyridin-6-yl]phenyl]-5-fluoro-2-methoxypyridine-3-sulfonamide

Into an 8 mL vial were added 2,4-difluoro-3-[1-(4-[[2-(trimethylsilyl)ethoxy]methyl]-1,2,4-triazol-3-yl) imidazo[1,5-a]pyridin-6-yl]aniline (150 mg, 0.34 mmol, 1 equiv), 5-chloro-2-methylpyridine-3-sulfonyl chloride (114 mg, 0.5 mmol, 1.5 equiv), DCM (5 mL) and pyridine (134 mg, 1.7 mmol, 5 equiv). The resulting mixture was stirred overnight at room temperature, then concentrated under vacuum. The residue was purified by silica gel column chromatography, eluting with PE/EtOAc (1:1) to afford N-[2,4-difluoro-3-[1-(4-[[2-(trimethylsilyl) ethoxy]methyl]-1,2,4-triazol-3-yl)imidazo[1,5-a]pyridin-6-yl]phenyl]-5-fluoro-2-methoxypyridine-3-sulfonamide (130 mg, 61% yield) as a light brown solid.

LCMS (ES, m/z): [M+H]⁺: 632

### Synthesis of Compound 70: N-[2,4-difluoro-3-[1-(4H-1,2,4-triazol-3-yl)imidazo[1,5-a]pyridin-6-yl]phenyl]-5-fluoro-2-methoxypyridine-3-sulfonamide

Into an 8 mL vial were added N-[2,4-difluoro-3-[1-(4-[[2-(trimethylsilyl)ethoxy]methyl]-1,2,4-triazol-3-yl)imidazo[1,5-a]pyridin-6-yl]phenyl]-5-fluoro-2-methoxypyridine-3-sulfonamide (120 mg, 0.19 mmol, 1 equiv) and TFA (2 mL). The resulting mixture was stirred for 30 min at 60° C., then concentrated under vacuum. The crude product was purified by Prep-HPLC with the following conditions: Column: Atlantis HILIC OBD, 19*150 mm*5 μm; Mobile Phase: 20-50% MeCN/0.1% aqueous formic acid; Flow rate: 90 mL/min; Detector 220 nm; to afford N-[2,4-difluoro-3-[1-(4H-1,2,4-triazol-3-yl)imidazo[1,5-a]pyridin-6-yl]phenyl]-5-fluoro-2-methoxypyridine-3-sulfonamide (37 mg, 39% yield) as a white solid.

LCMS (ES, m/z): [M+H]⁺: 502

¹H NMR (300 MHz, DMSO-d₆) δ 14.34 (s, 1H), 10.44 (s, 1H), 8.64-8.58 (m, 2H), 8.48 (d, J=3.0 Hz, 1H), 8.23 (d, J=9.5 Hz, 1H), 8.04 (dd, J=7.3, 3.0 Hz, 1H), 7.38 (td, J=8.8, 5.9 Hz, 1H), 7.32-7.20 (m, 1H), 7.01-6.95 (m, 1H), 3.92 (s, 3H).

### Example 86: Synthesis of 5-cyano-N-[2,4-difluoro-3-[1-(4H-1,2,4-triazol-3-yl)imidazo[1,5-a]pyridin-6-yl]phenyl]-2-methoxypyridine-3-sulfonamide (Compound 71)

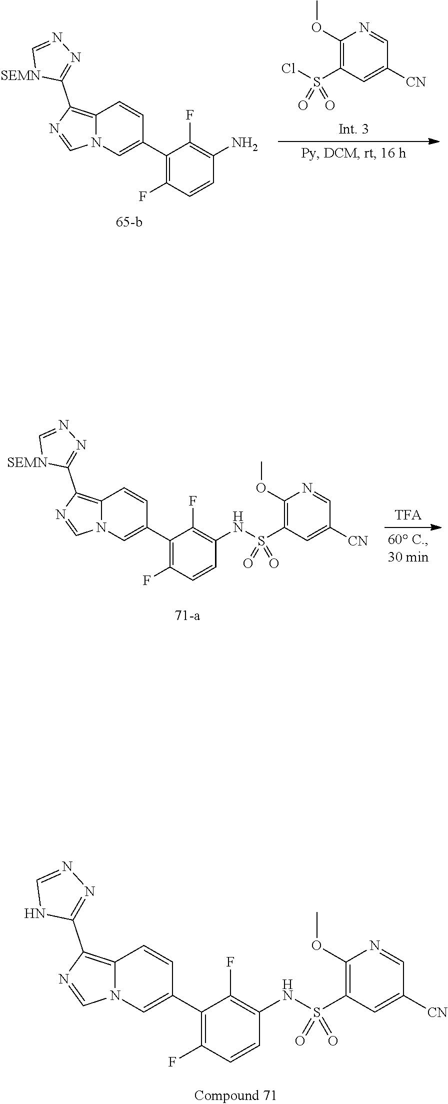 [SMILES: C[Si](C)(C)CCOCn1cnnc1-c1ncn2cc(-c3c(F)ccc(N)c3F)ccc12 ; COc1ncc(C#N)cc1S(=O)(=O)Cl ; COc1ncc(C#N)cc1S(=O)(=O)Nc1ccc(F)c(-c2ccc3c(-c4nncn4COCC[Si](C)(C)C)ncn3c2)c1F ; COc1ncc(C#N)cc1S(=O)(=O)Nc1ccc(F)c(-c2ccc3c(-c4nnc[nH]4)ncn3c2)c1F]

### Synthesis of 71-a: 5-cyano-N-[2,4-difluoro-3-[1-(4-[[2-(trimethylsilyl)ethoxy]methyl]-1,2,4-triazol-3-yl)imidazo[1,5-a]pyridin-6-yl]phenyl]-2-methoxypyridine-3-sulfonamide

Into an 8 mL vial were added 2,4-difluoro-3-[1-(4-[[2-(trimethylsilyl)ethoxy]methyl]-1,2,4-triazol-3-yl)imidazo[1,5-a]pyridin-6-yl]aniline (150 mg, 0.34 mmol, 1 equiv), 5-cyano-2-methoxypyridine-3-sulfonyl chloride (118 mg, 0.5 mmol, 1.5 equiv), DCM (5.00 mL) and pyridine (134 mg, 1.7 mmol, 5 equiv). The resulting mixture was stirred overnight at room temperature, then concentrated under vacuum. The residue was purified by silica gel column chromatography, eluting with PE/EtOAc (1:1) to afford 5-cyano-N-[2,4-difluoro-3-[1-(4-[[2-(trimethylsilyl)ethoxy]methyl]-1,2,4-triazol-3-yl)imidazo[1,5-a]pyridin-6-yl]phenyl]-2-methoxypyridine-3-sulfonamide (115 mg, 53% yield) as a light brown solid.

LCMS (ES, m/z): [M+H]⁺: 639

### Synthesis of Compound 71: 5-cyano-N-[2,4-difluoro-3-[1-(4H-1,2,4-triazol-3-yl)imidazo[1,5-a]pyridin-6-yl]phenyl]-2-methoxypyridine-3-sulfonamide

Into an 8 mL vial were added 5-cyano-N-[2,4-difluoro-3-[1-(4-[[2-(trimethylsilyl)ethoxy]methyl]-1,2,4-triazol-3-yl)imidazo[1,5-a]pyridin-6-yl]phenyl]-2-methoxypyridine-3-sulfonamide (105 mg, 0.16 mmol, 1 equiv) and TFA (2 mL). The resulting mixture was stirred for 30 min at 60° C., then concentrated under vacuum. The crude product was purified by Prep-HPLC with the following conditions: Column: Atlantis HILIC OBD, 19*150 mm*5 μm; Mobile Phase 20-50% MeCN/0.1% aqueous formic acid; Flow rate: 90 mL/min; Detector 220 nm; to afford 5-cyano-N-[2,4-difluoro-3-[1-(4H-1,2,4-triazol-3-yl)imidazo[1,5-a]pyridin-6-yl]phenyl]-2-methoxypyridine-3-sulfonamide (43 mg, 52% yield) as a white solid.

LCMS (ES, m/z): [M+H]⁺: 509.

¹H NMR (300 MHz, DMSO-d₆) δ14.34 (s, 1H), 10.55 (s, 1H), 8.95 (d, J=2.2 Hz, 1H), 8.60 (s, 2H), 8.52 (d, J=2.2 Hz, 1H), 8.23 (d, J=9.4 Hz, 1H), 7.40 (td, J=8.8, 5.8 Hz, 1H), 7.33-7.20 (m, 1H), 7.01-6.92 (m, 1H), 4.03 (s, 3H).

### Example 87: Synthesis of N-[2,4-difluoro-3-[1-(4H-1,2,4-triazol-3-yl)imidazo[1,5-a]pyridin-6-yl]phenyl]-5-fluoro-2-methylpyridine-3-sulfonamide (Compound 72)

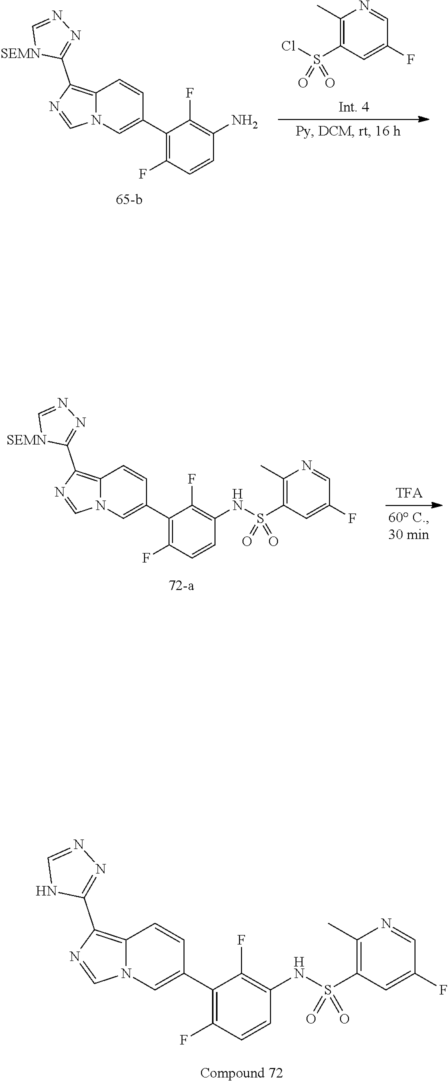 [SMILES: C[Si](C)(C)CCOCn1cnnc1-c1ncn2cc(-c3c(F)ccc(N)c3F)ccc12 ; Cc1ncc(F)cc1S(=O)(=O)Cl ; Cc1ncc(F)cc1S(=O)(=O)Nc1ccc(F)c(-c2ccc3c(-c4nncn4COCC[Si](C)(C)C)ncn3c2)c1F ; Cc1ncc(F)cc1S(=O)(=O)Nc1ccc(F)c(-c2ccc3c(-c4nnc[nH]4)ncn3c2)c1F]

### Synthesis of 72-a: 5-cyano-N-[2,4-difluoro-3-[1-(4-[[2-(trimethylsilyl)ethoxy]methyl]-1,2,4-triazol-3-yl) imidazo[1,5-a]pyridin-6-yl]phenyl]-2-methoxypyridine-3-sulfonamide

Into an 8 mL vial were added 2,4-difluoro-3-[1-(4-[[2-(trimethylsilyl)ethoxy]methyl]-1,2,4-triazol-3-yl)imidazo[1,5-a]pyridin-6-yl]aniline (150 mg, 0.34 mmol, 1 equiv), 5-fluoro-2-methylpyridine-3-sulfonyl chloride (213 mg, 1 mmol, 3 equiv), DCM (5 mL) and pyridine (134 mg, 1.7 mmol, 5 equiv). The resulting mixture was stirred overnight at room temperature, then concentrated under vacuum. The residue was purified by silica gel column chromatography, eluting with PE/EtOAc (1:1) to afford 5-cyano-N-[2,4-difluoro-3-[1-(4-[[2-(trimethylsilyl)ethoxy]methyl]-1,2,4-triazol-3-yl)imidazo[1,5-a]pyridin-6-yl]phenyl]-2-methoxypyridine-3-sulfonamide (115 mg, 53% yield) as a light brown solid.

LCMS (ES, m/z): [M+H]⁺: 616

### Synthesis of Compound 72: N-[2,4-difluoro-3-[1-(4H-1,2,4-triazol-3-yl)imidazo[1,5-a]pyridin-6-yl]phenyl]-5-fluoro-2-methylpyridine-3-sulfonamide

Into an 8 mL vial were added N-[2,4-difluoro-3-[1-(4-[[2-(trimethylsilyl)ethoxy]methyl]-1,2,4-triazol-3-yl)imidazo[1,5-a]pyridin-6-yl]phenyl]-5-fluoro-2-methylpyridine-3-sulfonamide (92 mg, 0.15 mmol, 1 equiv) and TFA (2 mL). The resulting mixture was stirred for 30 min at 60° C., then concentrated under vacuum. The crude product was purified by Prep-HPLC with the following conditions: Column: Atlantis HILIC OBD, 19*150 mm*5 μm; Mobile Phase: 20-50% MeCN/0.1% aqueous formic acid; Flow rate: 90 mL/min; Detector 220 nm; to afford N-[2,4-difluoro-3-[1-(4H-1,2,4-triazol-3-yl)imidazo[1,5-a]pyridin-6-yl]phenyl]-5-fluoro-2-methylpyridine-3-sulfonamide (27 mg, 37% yield) as a white solid.

LCMS (ES, m/z): [M+H]⁺: 486

¹H NMR (300 MHz, DMSO-d₆) δ 14.32 (s, 1H), 10.79 (s, 1H), 8.76 (d, J=2.8 Hz, 1H), 8.63-8.56 (m, 2H), 8.22 (d, J=9.5 Hz, 1H), 7.97 (dd, J=8.2, 2.8 Hz, 1H), 7.44-7.21 (m, 2H), 6.98-6.88 (m, 1H), 2.78 (d, J=1.2 Hz, 3H).

### Example 88: Synthesis of 5-chloro-N-[2,4-difluoro-3-[(6R)-1-(1H-imidazol-2-yl)-7-methyl-5H,6H,8H-imidazo[1,5-a]pyrazin-6-yl]phenyl]-2-methoxypyridine-3-sulfonamide and 5-chloro-N-[2,4-difluoro-3-[(6S)-1-(1H-imidazol-2-yl)-7-methyl-5H,6H,8H-imidazo[1,5-a]pyrazin-6-yl]phenyl]-2-methoxypyridine-3-sulfonamide (Compounds 73-1 & 73-2)

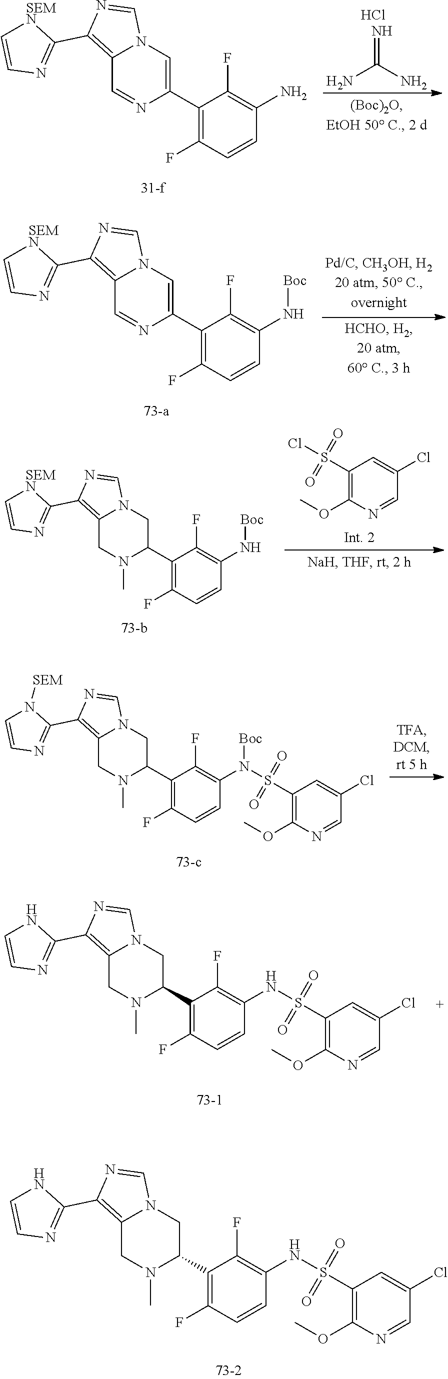

### Synthesis of 73-a: tert-butyl N-[2,4-difluoro-3-[1-(1-[[2-(trimethylsilyl)ethoxy]methyl]imidazol-2-yl) imidazo[1,5-a]pyrazin-6-yl]phenyl]carbamate

Into a 40 mL vial was placed 2,4-difluoro-3-[1-(1-[[2-(trimethylsilyl)ethoxy]methyl]imidazol-2-yl)imidazo[1,5-a]pyrazin-6-yl]aniline (560 mg, 1.2 mmol, 1 equiv), EtOH (6 mL), (Boc)₂O (552 mg, 2.5 mmol, 2 equiv) and guanidine hydrochloride (24 mg, 0.2 mmol, 0.2 equiv). The resulting solution was stirred for 2 days at 50° C., then cooled and concentrated. The residue was purified by prep-HPLC Flash-Prep-HPLC with the following conditions: Column, WelFlash™ C18-I, Spherical C18 20-40 m, 120 g; mobile phase: 5-60% MeCN/0.1% aqueous formic acid; Detector, 220 nm; to give tert-butyl N-[2,4-difluoro-3-[1-(1-[[2-(trimethylsilyl)ethoxy]methyl]imidazol-2-yl)imidazo[1,5-a]pyrazin-6-yl]phenyl]carbamate (490 mg, 71% yield) as a yellow solid.

LCMS (ES, m/z): [M+H]⁺: 543

### Synthesis 73-b: of tert-butyl-N-[2,4-difluoro-3-[7-methyl-1-(1-[[2-(trimethylsilyl)ethoxy]methyl]imidazol-2-yl)-5H,6H,8H-imidazo[1,5-a]pyrazin-6-yl]phenyl]carbamate

Tert-butyl-N-[2,4-difluoro-3-[1-(1-[[2-(trimethylsilyl)ethoxy]methyl]imidazol-2-yl)imidazo[1,5-a]pyrazin-6-yl]phenyl]carbamate (510 mg, 0.9 mmol, 1 equiv), 10% Pd/C (510 mg, 4.8 mmol, 5 equiv) and MeOH (10 mL) in a 50 mL sealed tube were hydrogenated under 20 atm at 50° C. overnight. HCHO (233 mg, 1.9 mmol, 2 equiv, 37% in water) was added and the resulting mixture was stirred under 20 atm hydrogen at 60° C. for a further 3 hrs. The reaction was cooled, filtered and concentrated under reduced pressure to give tert-butyl-N-[2,4-difluoro-3-[7-methyl-1-(1-[[2-(trimethylsilyl)ethoxy]methyl]imidazol-2-yl)-5H,6H,8H-imidazo[1,5-a]pyrazin-6-yl]phenyl]carbamate (410 mg, 78% yield) as a yellow solid.

LCMS (ES, m/z): [M+H]⁺: 561

### Synthesis of 73-c: tert-butyl-N-(5-chloro-2-methoxypyridin-3-ylsulfonyl)-N-[2,4-difluoro-3-[7-methyl-1-(1-[[2-(trimethylsilyl)ethoxy]methyl]imidazol-2-yl)-5H,6H,8H-imidazo[1,5-a]pyrazin-6-yl]phenyl]carbamate

To a stirred solution of tert-butyl-N-[2,4-difluoro-3-[7-methyl-1-(1-[[2-(trimethylsilyl)ethoxy]methyl]imidazol-2-yl)-5H,6H,8H-imidazo[1,5-a]pyrazin-6-yl]phenyl]carbamate (300 mg, 0.5 mmol, 1 equiv) in THE (7 mL) were added NaH (43 mg, 1 mmol, 2 equiv, 60% in oil) in portions at 0° C. 5-Chloro-2-methoxypyridine-3-sulfonyl chloride (155 mg, 0.6 mmol, 1.2 equiv) was added to the resulting solution in portions at 0° C., then the reaction was allowed to stir to room temperature over 2 hrs. The reaction was poured into 100 mL water/ice and extracted with EA (2×100 mL). The extracts were dried over anhydrous Na₂SO₄ and concentrated under reduced pressure to give tert-butyl N-(5-chloro-2-methoxypyridin-3-ylsulfonyl)-N-[2,4-difluoro-3-[7-methyl-1-(1-[[2-(trimethylsilyl)ethoxy]methyl]imidazol-2-yl)-5H,6H,8H-imidazo[1,5-a]pyrazin-6-yl]phenyl]carbamate (400 mg, 98% yield) as a yellow solid.

LCMS (ES, m/z): [M+H]⁺: 766

### Synthesis of Compound 73-1 & 73-2: 5-chloro-N-[2,4-difluoro-3-[(6R)-1-(1H-imidazol-2-yl)-7-methyl-5H,6H,8H-imidazo[1,5-a]pyrazin-6-yl]phenyl]-2-methoxypyridine-3-sulfonamide and 5-chloro-N-[2,4-difluoro-3-[(6S)-1-(1H-imidazol-2-yl)-7-methyl-5H,6H,8H-imidazo[1,5-a]pyrazin-6-yl]phenyl]-2-methoxypyridine-3-sulfonamide

Into a 50 mL round-bottom flask was placed tert-butyl N-(5-chloro-2-methoxypyridin-3-ylsulfonyl)-N-[2,4-difluoro-3-[7-methyl-1-(1-[[2-(trimethylsilyl)ethoxy]methyl]imidazol-2-yl)-5H,6H,8H-imidazo[1,5-a]pyrazin-6-yl]phenyl]carbamate (400 mg, 0.5 mmol, 1 equiv), DCM (6 mL) and TFA (2 mL). The resulting solution was stirred for 5 h at room temperature, then concentrated. The residue was dissolved in 3 mL of MeOH and the pH was adjusted to 8 with NH₃ (7 M in MeOH). This was purified by prep-HPLC with the following conditions: Column, welch Vltimate XB-C18, 50×250 mm, 10 μm; Mobile Phase: 5-35% MeCN/0.1% aqueous formic acid; Detector, 220 nm; and the enantiomers separated via chiral-Prep-HPLC with the following conditions: Column, CHIRALPAK IC-3, 4.6*50 mm, 3 μm; mobile phase: 50% EtOH (containing 0.1% diethylamine)/DCM; to give 5-chloro-N-[2,4-difluoro-3-[(6R)-1-(1H-imidazol-2-yl)-7-methyl-5H,6H,8H-imidazo[1,5-a]pyrazin-6-yl]phenyl]-2-methoxypyridine-3-sulfonamide (46 mg, 16% yield, stereochemistry randomly assigned) as an off-white solid and 5-chloro-N-[2,4-difluoro-3-[(6S)-1-(1H-imidazol-2-yl)-7-methyl-5H,6H,8H-imidazo[1,5-a]pyrazin-6-yl]phenyl]-2-methoxypyridine-3-sulfonamide (48 mg, 17% yield, stereochemistry randomly assigned) as an off-white solid.

LCMS (ES, m/z): [M+H]⁺: 536 for both isomers.

### 5-chloro-N-[2,4-difluoro-3-[(6R)-1-(1H-imidazol-2-yl)-7-methyl-5H,6H,8H-imidazo[1,5-a]pyrazin-6-yl]phenyl]-2-methoxypyridine-3-sulfonamide

¹H NMR (300 MHz, DMSO-d₆) δ 8.49 (d, J=2.6 Hz, 1H), 7.97 (d, J=2.6 Hz, 1H), 7.63 (s, 1H), 7.39 (td, J=8.8, 5.7 Hz, 1H), 7.15 (td, J=9.5, 9.0, 1.5 Hz, 1H), 6.97 (s, 2H), 4.40-4.22 (m, 2H), 4.19-3.99 (m, 2H), 3.94 (s, 3H), 3.56 (d, J=15.9 Hz, 1H), 2.01 (s, 3H).

### 5-chloro-N-[2,4-difluoro-3-[(6S)-1-(1H-imidazol-2-yl)-7-methyl-5H,6H,8H-imidazo[1,5-a]pyrazin-6-yl]phenyl]-2-methoxypyridine-3-sulfonamide

¹H NMR (300 MHz, DMSO-d₆) δ 8.49 (d, J=2.6 Hz, 1H), 7.96 (d, J=2.6 Hz, 1H), 7.63 (s, 1H), 7.39 (td, J=8.8, 5.7 Hz, 1H), 7.23-7.09 (m, 1H), 6.97 (s, 2H), 4.40-4.23 (m, 2H), 4.19-4.00 (m, 2H), 3.94 (s, 3H), 3.56 (d, J=15.9 Hz, 1H), 2.01 (s, 3H).

### Example 89: Synthesis of N-[2,4-difluoro-3-[1-(1,2,3,4-tetrazol-1-yl)imidazo[1,5-a]pyridin-6-yl]phenyl]-5-fluoro-2-methoxypyridine-3-sulfonamide (Compound 74)

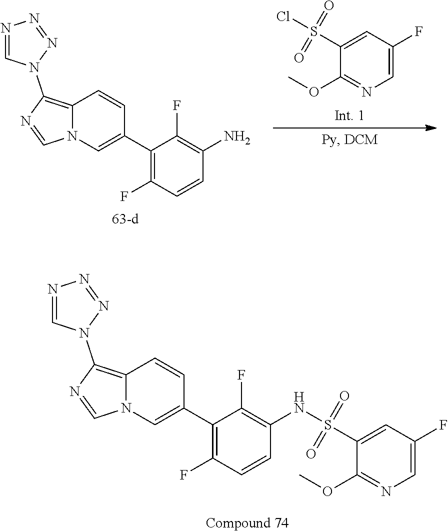 [SMILES: Nc1ccc(F)c(-c2ccc3c(-n4cnnn4)ncn3c2)c1F ; COc1ncc(F)cc1S(=O)(=O)Cl ; COc1ncc(F)cc1S(=O)(=O)Nc1ccc(F)c(-c2ccc3c(-n4cnnn4)ncn3c2)c1F]

### Synthesis of Compound 74: N-[2,4-difluoro-3-[1-(1,2,3,4-tetrazol-1-yl)imidazo[1,5-a]pyridin-6-yl]phenyl]-5-fluoro-2-methoxypyridine-3-sulfonamide

To a stirred solution of 2,4-difluoro-3-[1-(1,2,3,4-tetrazol-1-yl)imidazo[1,5-a]pyridin-6-yl]aniline (70 mg, 0.22 mmol, 1 equiv) and pyridine (71 mg, 0.9 mmol, 4 equiv) in DCM (1 mL) was added 5-fluoro-2-methoxypyridine-3-sulfonyl chloride (75 mg, 0.34 mmol, 1.5 equiv) dropwise at room temperature. The resulting mixture was stirred for 2 h at room temperature, then diluted with water (2 mL). The resulting mixture was extracted with EtOAc (3×10 mL), and the extracts were washed with water and brine, dried over anhydrous Na₂SO₄ before being concentrated under reduced pressure. The crude product was purified by Prep-HPLC with the following conditions: Column, XSelect CSH Prep C18 OBD, 5 μm, 19*150 mm; mobile phase: 34-55% MeCN/0.1% aqueous formic acid; Detector UV; to give N-[2,4-difluoro-3-[1-(1,2,3,4-tetrazol-1-yl)imidazo[1,5-a]pyridin-6-yl]phenyl]-5-fluoro-2-methoxypyridine-3-sulfonamide (22 mg, 69% yield) as an off-white solid.

LCMS (ES, m/z): [M+H]: 503.

¹H NMR (300 MHz, DMSO-d6) δ 10.49 (s, 1H), 10.04 (s, 1H), 8.65 (s, 2H), 8.43 (d, J=3.0 Hz, 1H), 8.01 (dd, J=7.4, 3.0 Hz, 1H), 7.90 (d, J=9.5 Hz, 1H), 7.43-7.29 (m, 1H), 7.21 (t, J=9.2 Hz, 1H), 7.03 (dd, J=9.3, 1.5 Hz, 1H), 3.89 (s, 3H).

### Example 90: Synthesis of 5-cyano-N-[2,4-difluoro-3-[1-(1,2,3,4-tetrazol-1-yl)imidazo[1,5-a]pyridin-6-yl]phenyl]-2-methoxypyridine-3-sulfonamide (Compound 75)

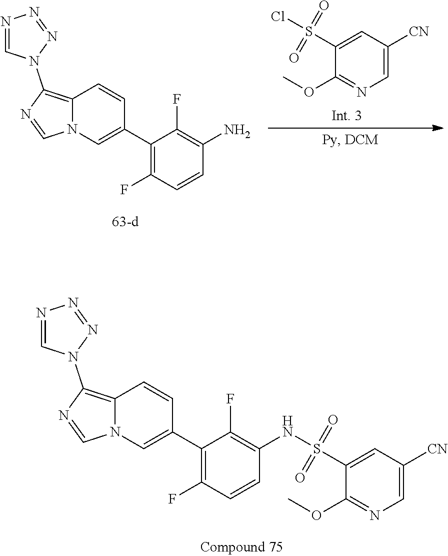 [SMILES: Nc1ccc(F)c(-c2ccc3c(-n4cnnn4)ncn3c2)c1F ; COc1ncc(C#N)cc1S(=O)(=O)Cl ; COc1ncc(C#N)cc1S(=O)(=O)Nc1ccc(F)c(-c2ccc3c(-n4cnnn4)ncn3c2)c1F]

### Synthesis Compound 75: of 5-cyano-N-[2,4-difluoro-3-[1-(1,2,3,4-tetrazol-1-yl)imidazo[1,5-a]pyridin-6-yl]phenyl]-2-methoxypyridine-3-sulfonamide

To a stirred solution of 2,4-difluoro-3-[1-(1,2,3,4-tetrazol-1-yl)imidazo[1,5-a]pyridin-6-yl]aniline (70 mg, 0.23 mmol, 1 equiv) and pyridine (71 mg, 0.9 mmol, 4 equiv) in DCM (1 mL) was added 5-cyano-2-methoxypyridine-3-sulfonyl chloride (78 mg, 0.34 mmol, 1.5 equiv) dropwise at room temperature. The resulting mixture was stirred for 2 h at room temperature, then diluted with water (2 mL). The resulting mixture was extracted with EtOAc (3×10 mL), and the extracts washed with water and brine, dried over anhydrous Na₂SO₄ and concentrated under reduced pressure. The crude product was purified by Prep-HPLC with the following conditions: Column, XSelect CSH Prep C18 OBD, 5 μm, 19*150 mm; mobile phase: 34-52% MeCN/0.1% aqueous formic acid; Detector, UV; to give 5-cyano-N-[2,4-difluoro-3-[1-(1,2,3,4-tetrazol-1-yl)imidazo[1,5-a]pyridin-6-yl]phenyl]-2-methoxypyridine-3-sulfonamide (37 mg, 46% yield) as an off-white solid.

LCMS (ES, m/z): [M+H]⁺: 510.

¹H NMR (300 MHz, DMSO-d₆) δ 10.59 (s, 1H), 10.04 (s, 1H), 8.93 (d, J=2.2 Hz, 1H), 8.65 (s, 2H), 8.50 (d, J=2.2 Hz, 1H), 7.90 (d, J=9.5 Hz, 1H), 7.39 (td, J=8.9, 5.8 Hz, 1H), 7.25 (td, J=9.2, 1.6 Hz, 1H), 7.07-6.97 (m, 1H), 4.02 (s, 3H).

### Example 91: Synthesis of N-[2,4-difluoro-3-[1-(1,2,3,4-tetrazol-1-yl)imidazo[1,5-a]pyridin-6-yl]phenyl]-5-fluoro-2-methylpyridine-3-sulfonamide (Compound 76)

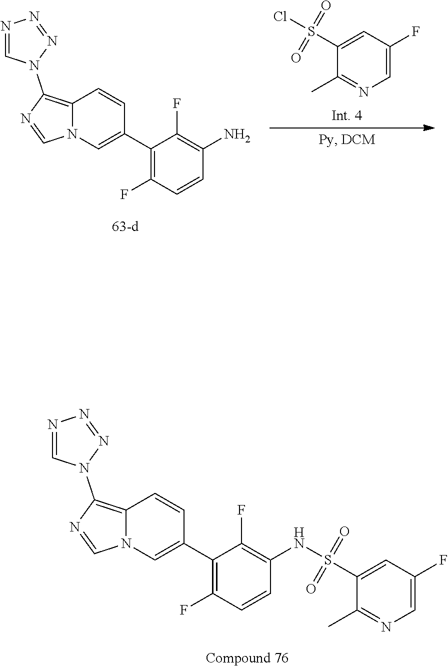 [SMILES: Nc1ccc(F)c(-c2ccc3c(-n4cnnn4)ncn3c2)c1F ; Cc1ncc(F)cc1S(=O)(=O)Cl ; Cc1ncc(F)cc1S(=O)(=O)Nc1ccc(F)c(-c2ccc3c(-n4cnnn4)ncn3c2)c1F]

### Synthesis of Compound 76: N-[2,4-difluoro-3-[1-(1,2,3,4-tetrazol-1-yl)imidazo[1,5-a]pyridin-6-yl]phenyl]-5-fluoro-2-methylpyridine-3-sulfonamide

To a stirred solution of 2,4-difluoro-3-[1-(1,2,3,4-tetrazol-1-yl)imidazo[1,5-a]pyridin-6-yl]aniline (70 mg, 0.22 mmol, 1 equiv) and pyridine (71 mg, 0.89 mmol, 4 equiv) in DCM (1 mL) was added 5-fluoro-2-methylpyridine-3-sulfonyl chloride (70 mg, 0.34 mmol, 1.5 equiv) dropwise at room temperature. The reaction was stirred for 2 h at room temperature, then diluted with water (2 mL). The resulting mixture was extracted with EtOAc (3×10 mL) and the extracts washed with water and brine, dried over anhydrous Na₂SO₄ and concentrated under reduced pressure. The crude product was purified by Prep-HPLC with the following conditions: Column, XSelect CSH Prep C18 OBD, 5 μm, 19*150 mm; mobile phase: 32-51% MeCN/0.1% aqueous formic acid; Detector, UV; to give N-[2,4-difluoro-3-[1-(1,2,3,4-tetrazol-1-yl)imidazo[1,5-a]pyridin-6-yl]phenyl]-5-fluoro-2-methylpyridine-3-sulfonamide (21 mg, 27% yield) as an off-white solid.

LCMS (ES, m/z): [M+H]⁺: 487.

¹H NMR (300 MHz, DMSO-d₆) δ 10.81 (s, 1H), 10.04 (s, 1H), 8.72 (d, J=2.9 Hz, 1H), 8.64 (d, J=2.1 Hz, 2H), 8.01-7.85 (m, 2H), 7.43-7.29 (m, 1H), 7.30-7.18 (m, 1H), 6.99 (dd, J=9.5, 1.5 Hz, 1H), 2.77 (d, J=1.2 Hz, 3H).

### Example 92: Synthesis of 5-chloro-N-[2,4-difluoro-3-[1-(1,2,3,4-tetrazol-1-yl)imidazo[1,5-a]pyridin-6-yl]phenyl]-2-methylpyridine-3-sulfonamide (Compound 77)

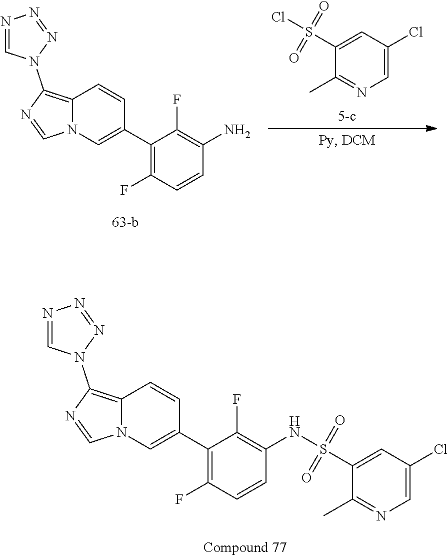 [SMILES: Nc1ccc(F)c(-c2ccc3c(-n4cnnn4)ncn3c2)c1F ; Cc1ncc(Cl)cc1S(=O)(=O)Cl ; Cc1ncc(Cl)cc1S(=O)(=O)Nc1ccc(F)c(-c2ccc3c(-n4cnnn4)ncn3c2)c1F]

### Synthesis of Compound 77: 5-chloro-N-[2,4-difluoro-3-[1-(1,2,3,4-tetrazol-1-yl)imidazo[1,5-a]pyridin-6-yl]phenyl]-2-methylpyridine-3-sulfonamide

To a stirred solution of 2,4-difluoro-3-[1-(1,2,3,4-tetrazol-1-yl)imidazo[1,5-a]pyridin-6-yl]aniline (70 mg, 0.23 mmol, 1 equiv) and pyridine (71 mg, 0.89 mmol, 4 equiv) in DCM (1 mL) was added 5-chloro-2-methylpyridine-3-sulfonyl chloride (71 mg, 0.34 mmol, 1.5 equiv) dropwise at room temperature. The resulting mixture was stirred for 2 h at room temperature, then diluted with water (2 mL). The resulting mixture was extracted with EtOAc (3×10 mL), and the extracts were washed with water and brine, dried over anhydrous Na₂SO₄, and concentrated under reduced pressure. The crude product was purified by Prep-HPLC with the following conditions: Column, XSelect CSH Prep C18 OBD, 5 μm, 19*150 mm; mobile phase: 37-54% MeCN/0.1% aqueous formic acid; Detector, UV; to give 5-chloro-N-[2,4-difluoro-3-[1-(1,2,3,4-tetrazol-1-yl)imidazo[1,5-a]pyridin-6-yl]phenyl]-2-methylpyridine-3-sulfonamide (30 mg, 27% yellow) as an off-white solid.

LCMS (ES, m/z): [M+H]⁺: 503.

¹H NMR (300 MHz, DMSO-d₆) δ 10.82 (s, 1H), 10.04 (s, 1H), 8.76 (d, J=2.4 Hz, 1H), 8.64 (s, 2H), 8.08 (d, J=2.4 Hz, 1H), 7.90 (d, J=9.5 Hz, 1H), 7.38 (td, J=8.9, 5.9 Hz, 1H), 7.26 (td, J=9.1, 1.5 Hz, 1H), 6.98 (dd, J=9.5, 1.5 Hz, 1H), 2.77 (s, 3H).

### Example 93: Synthesis of N-[2,4-difluoro-3-[1-(1H-imidazol-2-yl)imidazo[1,5-a]pyrazin-6-yl]phenyl]-5-fluoro-2-methoxypyridine-3-sulfonamide (Compound 78)

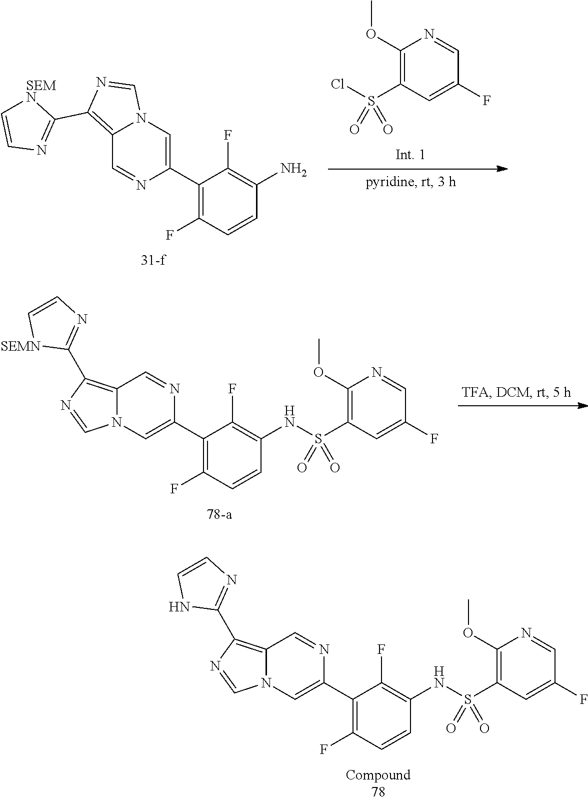 [SMILES: C[Si](C)(C)CCOCn1ccnc1-c1ncn2cc(-c3c(F)ccc(N)c3F)ncc12 ; COc1ncc(F)cc1S(=O)(=O)Cl ; COc1ncc(F)cc1S(=O)(=O)Nc1ccc(F)c(-c2cn3cnc(-c4nccn4COCC[Si](C)(C)C)c3cn2)c1F ; COc1ncc(F)cc1S(=O)(=O)Nc1ccc(F)c(-c2cn3cnc(-c4ncc[nH]4)c3cn2)c1F]

### Synthesis of 78-a: N-[2,4-difluoro-3-[1-(1-[[2-(trimethylsilyl)ethoxy]methyl]imidazol-2-yl)imidazo[1,5-a]pyrazin-6-yl]phenyl]-5-fluoro-2-methoxypyridine-3-sulfonamide

To a stirred solution of 2,4-difluoro-3-[1-(1-[[2-(trimethylsilyl)ethoxy]methyl]imidazol-2-yl)imidazo[1,5-a]pyrazin-6-yl]aniline (130 mg, 0.3 mmol, 1 equiv) in pyridine (3 mL) was added 5-fluoro-2-methoxypyridine-3-sulfonyl chloride (99 mg, 0.4 mmol, 1.5 equiv) in portions at room temperature. The resulting solution was stirred for 3 h, then concentrated under reduced pressure. The residue was purified by Flash-Prep-HPLC with the following conditions: Column, WelFlash™ C18-I, Spherical C18 20-40 m, 120 g; mobile phase, 5-70% MeCN/0.1% aqueous formic acid; Detector, 220 nm; to give N-[2,4-difluoro-3-[1-(1-[[2-(trimethylsilyl)ethoxy]methyl]imidazol-2-yl)imidazo[1,5-a]pyrazin-6-yl]phenyl]-5-fluoro-2-methoxypyridine-3-sulfonamide (100 mg, 54% yield) as a yellow solid.

LCMS (ES, m/z): [M+H]⁺: 632

### Synthesis of Compound 78: N-[2,4-difluoro-3-[1-(1H-imidazol-2-yl)imidazo[1,5-a]pyrazin-6-yl]phenyl]-5-fluoro-2-methoxypyridine-3-sulfonamide

Into an 8 mL vial, was placed N-[2,4-difluoro-3-[1-(1-[[2-(trimethylsilyl)ethoxy]methyl]imidazol-2-yl)imidazo[1,5-a]pyrazin-6-yl]phenyl]-5-fluoro-2-methoxypyridine-3-sulfonamide (100 mg, 0.2 mmol, 1 equiv), DCM (1 mL) and TFA (3 mL). The resulting solution was stirred for 5 h at 25° C., then was concentrated. The residue was diluted with 3 mL of MeOH and the pH adjusted to 8 with NH₃ (7 M in MeOH). This was purified by prep-HPLC with the following conditions: Column, welch Vltimate XB-C18, 50×250 mm, 10 μm; Mobile Phase: 5-40% MeCN/0.1% aqueous formic acid; Detector, 220 nm; to give N-[2,4-difluoro-3-[1-(1H-imidazol-2-yl)imidazo[1,5-a]pyrazin-6-yl]phenyl]-5-fluoro-2-methoxypyridine-3-sulfonamide (45 mg, 57% yield) as a yellow solid.

LCMS (ES, m/z): [M+H]⁺: 502 H NMR (300 MHz, DMSO-d₆) δ 12.73 (br s, 1H), 10.44 (br s, 1H), 9.55 (d, J=1.6 Hz, 1H), 8.66 (s, 1H), 8.56 (t, J=1.3 Hz, 1H), 8.47 (d, J=2.9 Hz, 1H), 8.03 (dd, J=7.3, 3.0 Hz, 1H), 7.40 (td, J=8.9, 5.8 Hz, 1H), 7.31-7.04 (m, 3H), 3.92 (s, 3H).

### Example 94: Synthesis of 5-chloro-N-[2,4-difluoro-3-[(6R)-1-(1H-imidazol-2-yl)-5H,6H,7H,8H-imidazo[1,5-a]pyridin-6-yl]phenyl]-2-methoxypyridine-3-sulfonamide and 5-chloro-N-[2,4-difluoro-3-[(6S)-1-(1H-imidazol-2-yl)-5H,6H,7H,8H-imidazo[1,5-a]pyridin-6-yl]phenyl]-2-methoxypyridine-3-sulfonamide (Compounds 79-1 & 79-2)

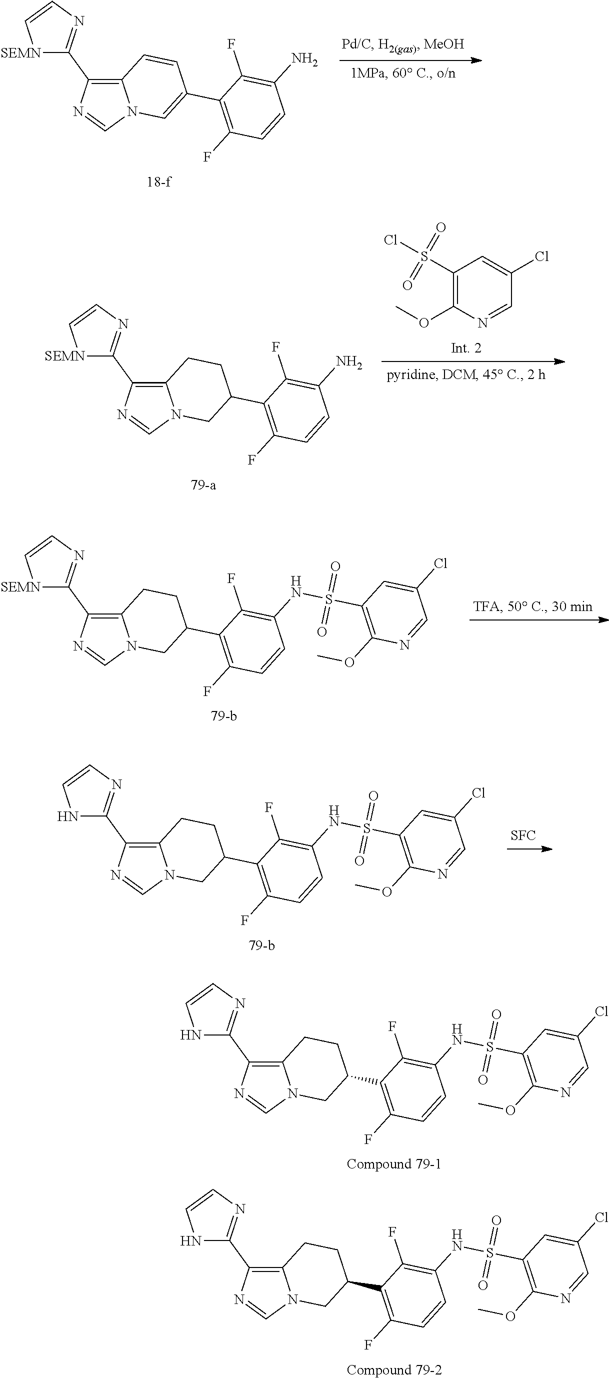

### Synthesis of 79-a: 2,4-difluoro-3-[1-(1-[[2-(trimethylsilyl)ethoxy]methyl]imidazol-2-yl)-5H,6H,7H,8H-imidazo[1,5-a]pyridin-6-yl]aniline

Into a 30 mL pressure tank reactor was placed 2,4-difluoro-3-[1-(1-[[2-(trimethylsilyl)ethoxy]methyl]imidazol-2-yl)imidazo[1,5-a]pyridin-6-yl]aniline (2.1 g, 5 mmol, 1 equiv), 10% Pd/C (1 g) and MeOH (20 mL), followed by an atmosphere of hydrogen. This was stirred overnight at 60° C., then cooled and filtered. The filtrate was concentrated to give 2,4-difluoro-3-[1-(1-[[2-(trimethylsilyl)ethoxy]methyl]imidazol-2-yl)-5H,6H,7H,8H-imidazo[1,5-a]pyridin-6-yl]aniline (2.0 g, 85% yield) as a brown solid.

LCMS (ES, m/z): [M+H]⁺: 446

### Synthesis of 79-b: 5-chloro-N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-2-methoxypyridine-3-sulfonamide

Into a 25 mL 3-necked round-bottom flask was placed 2,4-difluoro-3-[1-(1-[[2-(trimethylsilyl)ethoxy]methyl]imidazol-2-yl)-5H,6H,7H,8H-imidazo[1,5-a]pyridin-6-yl]aniline (400 mg, 0.9 mmol, 1 equiv), 5-chloro-2-methoxypyridine-3-sulfonyl chloride (217 mg, 0.9 mmol, 1 equiv) and pyridine (213 mg, 2.7 mmol, 3 equiv) in DCM (10 mL). The resulting solution was stirred for 2 h at 45° C., then concentrated under vacuum. The crude product was purified by prep-HPLC with the following conditions: Column, welch Vltimate XB-C18, 50×250 mm; Mobile Phase: 5-40% MeCN/0.1% aqueous formic acid; Detector, 220 nm; to give 5-chloro-N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-2-methoxypyridine-3-sulfonamide (190 mg, 29% yield) as a white solid.

LCMS (ES, m/z): [M+H]⁺: 651

### Synthesis of Compounds 79-1 & 79-2: 5-chloro-N-[2,4-difluoro-3-[(6R)-1-(1H-imidazol-2-yl)-5H,6H,7H,8H-imidazo[1,5-a]pyridin-6-yl]phenyl]-2-methoxypyridine-3-sulfonamide and 5-chloro-N-[2,4-difluoro-3-[(6S)-1-(1H-imidazol-2-yl)-5H,6H,7H,8H-imidazo[1,5-a]pyridin-6-yl]phenyl]-2-methoxypyridine-3-sulfonamide

Into a 25 mL 3-necked round-bottom flask was placed 5-chloro-N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-2-methoxypyridine-3-sulfonamide (190 mg, 0.3 mmol, 1 equiv) and TFA (2 mL). The resulting solution was stirred for 30 min at 50° C., then concentrated under vacuum. The pH was adjusted to 10 with ammonia and the crude product purified by prep-HPLC with the following conditions: Column, welch Vltimate XB-C18, 50×250 mm, 5 m; Mobile Phase: 5-35% MeCN/0.05% aqueous ammonia; Detector, 220 nm; to give 5-chloro-N-[2,4-difluoro-3-[1-(1H-imidazol-2-yl)-5H,6H,7H,8H-imidazo[1,5-a]pyridin-6-yl]phenyl]-2-methoxypyridine-3-sulfonamide (83 mg, 49% yield) as a white solid.

The enantiomers were separated by SFC with the following conditions: Column, CHIRALPAK IC, 20*250 mm, 5 m; Mobile Phase: 50% 1:1 MeOH/EtOH/Hexane containing 0.1% diethylamine; to give 5-chloro-N-[2,4-difluoro-3-[(6R)-1-(1H-imidazol-2-yl)-5H,6H,7H,8H-imidazo[1,5-a]pyridin-6-yl]phenyl]-2-methoxypyridine-3-sulfonamide (19 mg, 23% yield, stereochemistry randomly assigned) as a white solid, and 5-chloro-N-[2,4-difluoro-3-[(6S)-1-(1H-imidazol-2-yl)-5H,6H,7H,8H-imidazo[1,5-a]pyridin-6-yl]phenyl]-2-methoxypyridine-3-sulfonamide (20 mg, 23% yield, stereochemistry randomly assigned) as a white solid.

### 5-chloro-N-[2,4-difluoro-3-[(6R)-1-(1H-imidazol-2-yl)-5H,6H,7H,8H-imidazo[1,5-a]pyridin-6-yl]phenyl]-2-methoxypyridine-3-sulfonamide

LCMS (ES, m/z): [M+H]⁺: 521

¹H NMR (300 MHz, DMSO-d₆) δ 8.49 (d, J=2.6 Hz, 1H), 8.02 (d, J=2.5 Hz, 1H), 7.59 (s, 1H), 7.25 (td, J=8.9, 5.8 Hz, 1H), 7.15-7.02 (m, 1H), 6.95 (s, 2H), 4.29 (dd, J=12.3, 5.1 Hz, 1H), 4.00 (t, J=11.9 Hz, 1H), 3.92 (s, 3H), 3.60-3.35 (m, 2H), 2.97-2.81 (m, 1H), 2.12 (d, J=10.3 Hz, 1H), 2.05-1.90 (m, 1H). 5-chloro-N-[2,4-difluoro-3-[(6S)-1-(1H-imidazol-2-yl)-5H,6H,7H,8H-imidazo[1,5-a]pyridin-6-yl]phenyl]-2-methoxypyridine-3-sulfonamide

LCMS (ES, m/z): [M+H]⁺: 521

¹H NMR (300 MHz, DMSO-d₆) δ 8.49 (d, J=2.6 Hz, 1H), 8.02 (d, J=2.6 Hz, 1H), 7.59 (s, 1H), 7.32-7.18 (m, 1H), 7.08 (t, J=9.5 Hz, 1H), 6.95 (s, 2H), 4.29 (dd, J=12.3, 5.2 Hz, 1H), 4.00 (t, J=11.9 Hz, 1H), 3.92 (s, 3H), 3.60-3.35 (m, 2H), 2.96-2.86 (m, 1H), 2.13 (d, J=13.2 Hz, 1H), 1.90-2.20 (m, 1H).

### Example 95: Synthesis of N-[2,4-difluoro-3-[(6R)-1-(1H-imidazol-2-yl)-5H,6H,7H,8H-imidazo[1,5-a]pyridin-6-yl]phenyl]-5-fluoro-2-methylpyridine-3-sulfonamide and N-[2,4-difluoro-3-[(6S)-1-(1H-imidazol-2-yl)-5H,6H,7H,8H-imidazo[1,5-a]pyridin-6-yl]phenyl]-5-fluoro-2-methylpyridine-3-sulfonamide (Compounds 80-1 & 80-2)

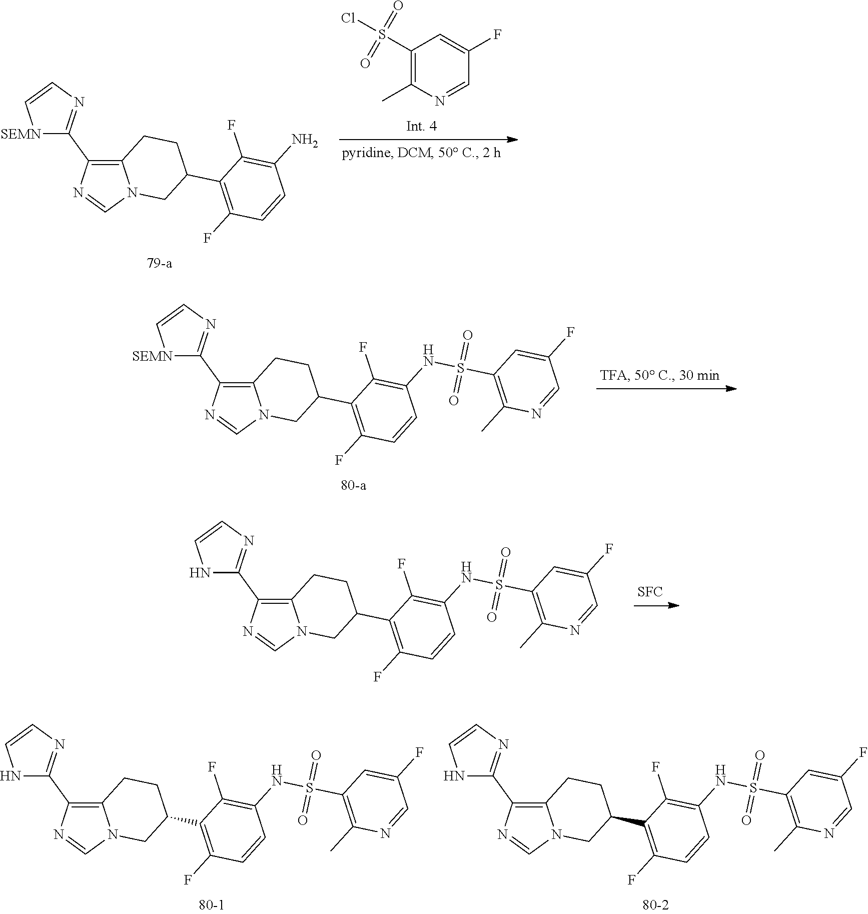

### Synthesis of 80-a: N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-5-fluoro-2-methylpyridine-3-sulfonamide

Into a 25 mL 3-necked round-bottom flask was placed 2,4-difluoro-3-[1-(1-[[2-(trimethylsilyl)ethoxy]methyl]imidazol-2-yl)-5H,6H,7H,8H-imidazo[1,5-a]pyridin-6-yl]aniline (400 mg, 0.9 mmol, 1 equiv), 5-fluoro-2-methylpyridine-3-sulfonyl chloride (188 mg, 0.9 mmol, 1 equiv) and pyridine (213 mg, 2.7 mmol, 3 equiv) in DCM (10 mL). The resulting solution was stirred for 2 h at 45° C., then concentrated under vacuum. The crude product was purified by Flash-Prep-HPLC with the following conditions: welch Vltimate XB-C18, 50×250 mm, 10 m; Mobile Phase: 20-65% MeCN/0.05% aqueous ammonia; Detector, 220 nm; to give N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-5-fluoro-2-methyl pyridine-3-sulfonamide (132 mg, 25% yield) as a white solid.

LCMS (ES, m/z): [M+H]⁺: 619

### Synthesis of Compounds 80-1 & 80-2: N-[2,4-difluoro-3-[(6R)-1-(1H-imidazol-2-yl)-5H,6H,7H,8H-imidazo[1,5-a]pyridin-6-yl]phenyl]-5-fluoro-2-methylpyridine-3-sulfonamide and N-[2,4-difluoro-3-[(6S)-1-(1H-imidazol-2-yl)-5H,6H,7H,8H-imidazo[1,5-a]pyridin-6-yl]phenyl]-5-fluoro-2-methylpyridine-3-sulfonamide

Into a 25 mL 3-necked round-bottom flask was placed N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-5-fluoro-2-methylpyridine-3-sulfonamide (132 mg, 0.2 mmol, 1 equiv) and TFA (2 mL). The resulting solution was stirred for 30 min at 50° C., then concentrated under vacuum. The pH was adjusted to 10 with NH₃·H₂O and this purified by Flash-Prep-HPLC with the following conditions: Column, welch Vltimate XB-C18, 50×250 mm, 10 m; Mobile Phase: 5-30% MeCN/0.05% aqueous ammonia; Detector, 220 nm; to give N-[2,4-difluoro-3-[1-(1H-imidazol-2-yl)-5H,6H,7H,8H-imidazo[1,5-a]pyridin-6-yl]phenyl]-5-fluoro-2-methylpyridine-3-sulfonamide (72 mg, 62% yield) as a white solid.

The enantiomers were separated by SFC with the following conditions: Column, CHIRALPAK IC, 20*250 mm, 5 m; Mobile Phase: 50% 1:1 MeOH/EtOH/3:1 Hexane/DCM containing 0.1% diethylamine; Detector, 220 nm; to give N-[2,4-difluoro-3-[(6R)-1-(1H-imidazol-2-yl)-5H,6H,7H,8H-imidazo[1,5-a]pyridin-6-yl]phenyl]-5-fluoro-2-methyl pyridine-3-sulfonamide (20 mg, stereochemistry randomly assigned) as a white solid and N-[2,4-difluoro-3-[(6S)-1-(1H-imidazol-2-yl)-5H,6H,7H,8H-imidazo[1,5-a]pyridin-6-yl]phenyl]-5-fluoro-2-methyl pyridine-3-sulfonamide (19 mg, stereochemistry randomly assigned).

### N-[2,4-difluoro-3-[(6R)-1-(1H-imidazol-2-yl)-5H,6H,7H,8H-imidazo[1,5-a]pyridin-6-yl]phenyl]-5-fluoro-2-methyl pyridine-3-sulfonamide

LCMS (ES, m/z): [M+H]⁺: 489

¹H NMR (300 MHz, DMSO-d₆) δ 8.71 (d, J=2.8 Hz, 1H), 7.87 (dd, J=8.3, 2.9 Hz, 1H), 7.60 (s, 1H), 7.22 (td, J=9.0, 5.8 Hz, 1H), 7.07 (t, J=9.6 Hz, 1H), 7.00 (s, 2H), 4.26 (dd, J=12.2, 5.1 Hz, 1H), 3.97 (t, J=11.8 Hz, 1H), 3.45 (d, J=28.1 Hz, 2H), 2.91 (q, J=11.0, 9.6 Hz, 1H), 2.74 (d, J=1.2 Hz, 3H), 2.11-1.92 (m, 2H).

### N-[2,4-difluoro-3-[(6S)-1-(1H-imidazol-2-yl)-5H,6H,7H,8H-imidazo[1,5-a]pyridin-6-yl]phenyl]-5-fluoro-2-methyl pyridine-3-sulfonamide

LCMS (ES, m/z): [M+H]⁺: 489

¹H NMR (300 MHz, DMSO-d₆) δ 8.71 (d, J=2.8 Hz, 1H), 7.87 (dd, J=8.3, 2.8 Hz, 1H), 7.60 (s, 1H), 7.23 (td, J=8.9, 5.8 Hz, 1H), 7.08 (t, J=9.4 Hz, 1H), 7.01 (s, 2H), 4.26 (dd, J=12.3, 5.1 Hz, 1H), 4.04-3.89 (m, 1H), 3.49 (s, 2H), 2.90 (td, J=11.4, 5.9 Hz, 1H), 2.74 (d, J=1.2 Hz, 3H), 2.08 (d, J=8.0 Hz, 1H), 1.99 (s, 1H).

### Example 96: Synthesis of N-[2,4-difluoro-3-[(6R)-1-(1H-imidazol-2-yl)-5H,6H,7H,8H-imidazo[1,5-a]pyridin-6-yl]phenyl]-5-fluoro-2-methoxypyridine-3-sulfonamide and N-[2,4-difluoro-3-[(6S)-1-(1H-imidazol-2-yl)-5H,6H,7H,8H-imidazo[1,5-a]pyridin-6-yl]phenyl]-5-fluoro-2-methoxypyridine-3-sulfonamide (Compounds 81-1 & 81-2)

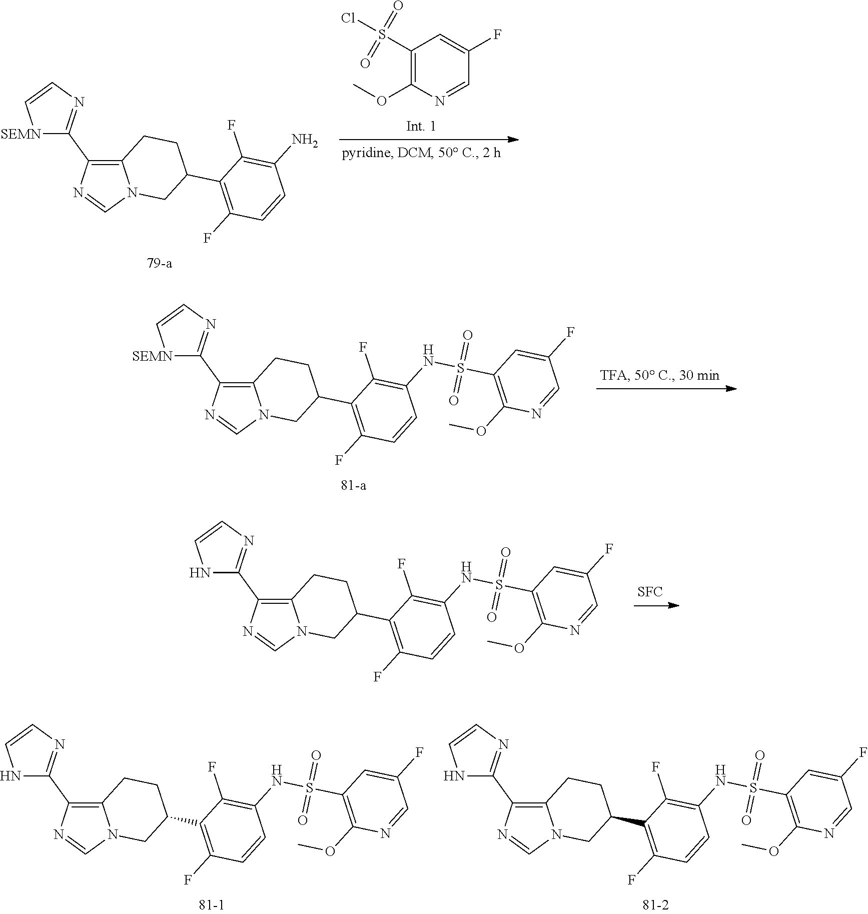

### Synthesis of 81-a: N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-5-fluoro-2-methoxypyridine-3-sulfonamide

Into a 25 mL 3-necked round-bottom flask, was placed 2,4-difluoro-3-[1-(1-[[2-(trimethylsilyl)ethoxy]methyl]imidazol-2-yl)-5H,6H,7H,8H-imidazo[1,5-a]pyridin-6-yl]aniline (400 mg, 0.9 mmol, 1 equiv), 5-fluoro-2-methoxypyridine-3-sulfonyl chloride (202 mg, 0.9 mmol, 1 equiv) and pyridine (213 mg, 2.7 mmol, 3 equiv) in DCM (10 mL). The resulting solution was stirred for 2 h at 45° C., then concentrated under vacuum. The crude product was purified by Flash-Prep-HPLC with the following conditions: Column, Sunfire Prep C18 OBD, 50*250 mm, 5 m; mobile phase: 10-50% MeCN/0.1% aqueous formic acid; Detector 220 nm; to give N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-5-fluoro-2-methoxypyridine-3-sulfonamide (230 mg, 36% yield) as a white solid.

LCMS (ES, m/z): [M+H]⁺: 635

### Synthesis of Compounds 81-1 & 81-2: N-[2,4-difluoro-3-[(6R)-1-(1H-imidazol-2-yl)-5H,6H,7H,8H-imidazo[1,5-a]pyridin-6-yl]phenyl]-5-fluoro-2-methoxypyridine-3-sulfonamide and N-[2,4-difluoro-3-[(6S)-1-(1H-imidazol-2-yl)-5H,6H,7H,8H-imidazo[1,5-a]pyridin-6-yl]phenyl]-5-fluoro-2-methoxypyridine-3-sulfonamide

Into a 25 mL 3-necked round-bottom flask, was placed N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-5-fluoro-2-methoxypyridine-3-sulfonamide (230 mg, 0.36 mmol, 1 equiv) and TFA (2 mL). The resulting solution was stirred for 30 min at 50° C., then concentrated under vacuum. The pH was adjusted to 10 with ammonia, and this was purified by Flash-Prep-HPLC with the following conditions: Column, welch xtimate C18, 50*250 mm, 5 m; mobile phase: 5-30% MeCN/0.05% aqueous ammonia; Detector 220 nm; to give N-[2,4-difluoro-3-[1-(1H-imidazol-2-yl)-5H,6H,7H,8H-imidazo[1,5-a]pyridin-6-yl]phenyl]-5-fluoro-2-methoxypyridine-3-sulfonamide (120 mg, 62% yield) as a white solid.

The enantiomers were separated by SFC with the following conditions: Column, CHIRALPAK IC, 20*250 mm, 5 m, Mobile Phase: 1:1 MeOH/EtOH/50% Hexane containing 0.1% diethylamine; Detector, 220 nm; to give N-[2,4-difluoro-3-[(6R)-1-(1H-imidazol-2-yl)-5H,6H,7H,8H-imidazo[1,5-a]pyridin-6-yl]phenyl]-5-fluoro-2-methoxypyridine-3-sulfonamide (27 mg, 22% yield, stereochemistry randomly assigned) as a white solid and N-[2,4-difluoro-3-[(6S)-1-(1H-imidazol-2-yl)-5H,6H,7H,8H-imidazo[1,5-a]pyridin-6-yl]phenyl]-5-fluoro-2-methoxypyridine-3-sulfonamide (27 mg, 22% yield, stereochemistry randomly assigned).

### N-[2,4-difluoro-3-[(6R)-1-(1H-imidazol-2-yl)-5H,6H,7H,8H-imidazo[1,5-a]pyridin-6-yl]phenyl]-5-fluoro-2-methoxypyridine-3-sulfonamide

LCMS (ES, m/z): [M+H]⁺: 505

¹H NMR (300 MHz, DMSO-d₆) δ 8.46 (d, J=3.0 Hz, 1H), 7.97 (dd, J=7.3, 3.0 Hz, 1H), 7.59 (s, 1H), 7.25 (td, J=8.9, 5.8 Hz, 1H), 7.16-7.03 (m, 1H), 6.96 (s, 2H), 4.29 (dd, J=12.2, 5.2 Hz, 1H), 4.00 (t, J=11.9 Hz, 1H), 3.92 (s, 3H), 3.60-3.30 (m, 2H), 2.90 (ddd, J=17.5, 12.0, 6.2 Hz, 1H), 2.20-1.90 (m, 2H).

### N-[2,4-difluoro-3-[(6S)-1-(1H-imidazol-2-yl)-5H,6H,7H,8H-imidazo[1,5-a]pyridin-6-yl]phenyl]-5-fluoro-2-methoxypyridine-3-sulfonamide

LCMS (ES, m/z): [M+H]⁺: 505

¹H NMR (300 MHz, DMSO-d₆) δ 8.46 (d, J=3.0 Hz, 1H), 7.97 (dd, J=7.3, 3.0 Hz, 1H), 7.59 (s, 1H), 7.25 (td, J=8.8, 5.7 Hz, 1H), 7.16-7.03 (m, 1H), 6.96 (s, 2H), 4.29 (dd, J=12.3, 5.2 Hz, 1H), 4.07-3.94 (m, 1H), 3.92 (s, 3H), 3.60-3.30 (m, 2H), 2.90 (ddd, J=17.8, 11.7, 6.2 Hz, 1H), 2.20-1.90 (m, 2H).

### Example 97: Synthesis of 5-cyano-N-[2,4-difluoro-3-[(6R)-1-(1H-imidazol-2-yl)-5H,6H,7H,8H-imidazo[1,5-a]pyridin-6-yl]phenyl]-2-methoxypyridine-3-sulfonamide and 5-cyano-N-[2,4-difluoro-3-[(6S)-1-(1H-imidazol-2-yl)-5H,6H,7H,8H-imidazo[1,5-a]pyridin-6-yl]phenyl]-2-methoxypyridine-3-sulfonamide (Compounds 82-1 & 82-2)

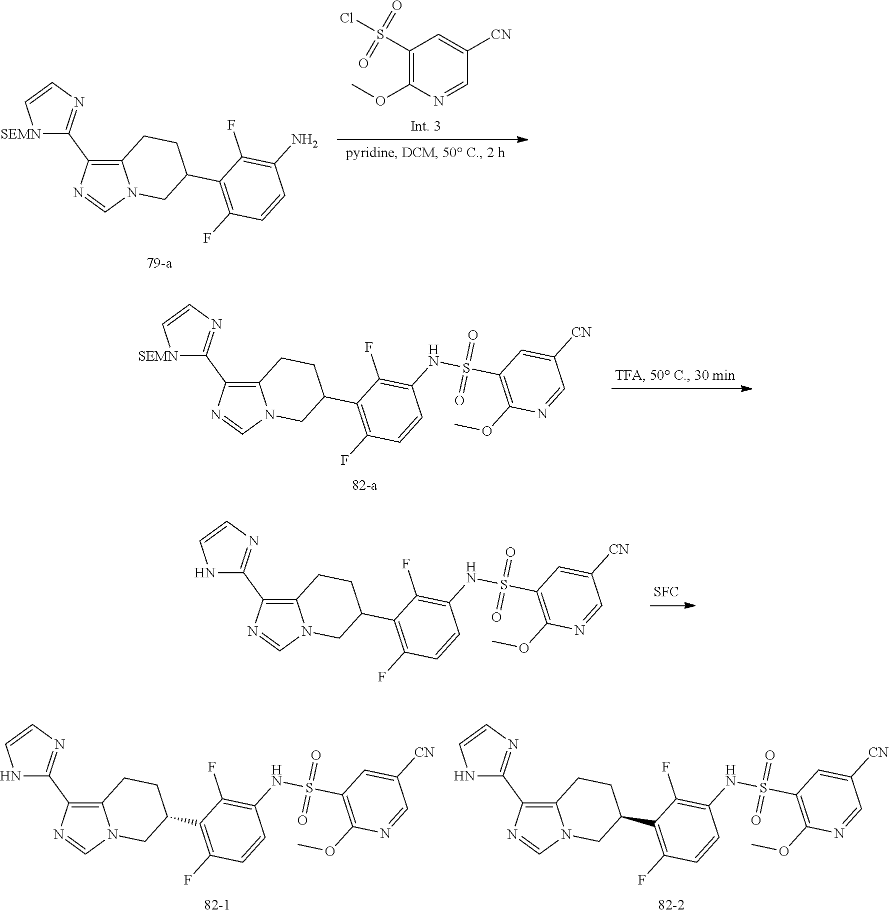

### Synthesis of 82-a: 5-cyano-N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-2-methoxypyridine-3-sulfonamide

Into a 25 mL 3-necked round-bottom flask was placed 2,4-difluoro-3-[1-(1-[[2-(trimethylsilyl)ethoxy]methyl]imidazol-2-yl)-5H,6H,7H,8H-imidazo[1,5-a]pyridin-6-yl]aniline (400 mg, 0.9 mmol, 1 equiv), DCM (10 mL), 5-cyano-2-methoxypyridine-3-sulfonyl chloride (209 mg, 0.9 mmol, 1 equiv) and pyridine (213 mg, 2.7 mmol, 3 equiv). The resulting solution was stirred for 2 h at 45° C., then quenched by the addition of 2 mL of CH₃OH. The resulting mixture was concentrated under vacuum, and the crude product was purified by Flash-Prep-HPLC with the following conditions: Column, C18 silica gel; mobile phase, 10-50% MeCN/0.1% aqueous formic acid; Detector, 220 nm; to give 5-cyano-N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-2-methoxypyridine-3-sulfonamide (237 mg, 39% yield) as a white solid.

LCMS (ES, m/z): [M+H]⁺: 642

### Synthesis of Compounds 82-1 & 82-2: 5-cyano-N-[2,4-difluoro-3-[(6R)-1-(1H-imidazol-2-yl)-5H,6H,7H,8H-imidazo[1,5-a]pyridin-6-yl]phenyl]-2-methoxypyridine-3-sulfonamide and 5-cyano-N-[2,4-difluoro-3-[(6S)-1-(1H-imidazol-2-yl)-5H,6H,7H,8H-imidazo[1,5-a]pyridin-6-yl]phenyl]-2-methoxypyridine-3-sulfonamide

Into a 25 mL 3-necked round-bottom flask, was placed 5-cyano-N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-2-methoxypyridine-3-sulfonamide (237 mg, 0.37 mmol, 1 equiv) and TFA (2 mL). The resulting solution was stirred for 30 min at 50° C., then concentrated under vacuum. The pH was adjusted to 10 with ammonia, and this was purified by Flash-Prep-HPLC with the following conditions: Column, C18; mobile phase: 5-35% MeCN/0.05% aqueous ammonia; Detector, 220 nm; to give 5-cyano-N-[2,4-difluoro-3-[1-(1H-imidazol-2-yl)-5H,6H,7H,8H-imidazo[1,5-a]pyridin-6-yl]phenyl]-2-methoxypyridine-3-sulfonamide (120 mg, 57% yield) as a white solid.

The enantiomers were separated by SFC with the following conditions: Column, CHIRALPAK IC, 20*250 mm, 5 m; Mobile Phase: 50% 1:1 MeOH:EtOH/3:1 Hexane:DCM containing 0.1% diethylamine; Detector, 220 nm; to give 5-cyano-N-[2,4-difluoro-3-[(6R)-1-(1H-imidazol-2-yl)-5H,6H,7H,8H-imidazo[1,5-a]pyridin-6-yl]phenyl]-2-methoxypyridine-3-sulfonamide (20 mg, stereochemistry randomly assigned) as a white solid and 5-cyano-N-[2,4-difluoro-3-[(6S)-1-(1H-imidazol-2-yl)-5H,6H,7H,8H-imidazo[1,5-a]pyridin-6-yl]phenyl]-2-methoxypyridine-3-sulfonamide (20 mg, stereochemistry randomly assigned) as a white solid.

### 5-cyano-N-[2,4-difluoro-3-[(6R)-1-(1H-imidazol-2-yl)-5H,6H,7H,8H-imidazo[1,5-a]pyridin-6-yl]phenyl]-2-methoxypyridine-3-sulfonamide

LCMS (ES, m/z): [M+H]⁺: 512

¹H NMR (300 MHz, DMSO-d₆) δ 8.92 (d, J=2.2 Hz, 1H), 8.43 (d, J=2.3 Hz, 1H), 7.59 (s, 1H), 7.31-7.21 (m, 1H), 7.09 (t, J=9.4 Hz, 1H), 6.97 (s, 2H), 4.29 (dd, J=12.1, 5.1 Hz, 1H), 4.02 (s, 4H), 3.46 (d, J=30.2 Hz, 2H), 2.92 (d, J=10.3 Hz, 1H), 2.06 (d, J=26.6 Hz, 2H).

### 5-cyano-N-[2,4-difluoro-3-[(6S)-1-(1H-imidazol-2-yl)-5H,6H,7H,8H-imidazo[1,5-a]pyridin-6-yl]phenyl]-2-methoxypyridine-3-sulfonamide

LCMS (ES, m/z): [M+H]⁺: 512

¹H NMR (300 MHz, DMSO-d₆) δ 8.93 (d, J=2.2 Hz, 1H), 8.44 (d, J=2.2 Hz, 1H), 7.61 (s, 1H), 7.27 (td, J=8.9, 5.9 Hz, 1H), 7.11 (t, J=9.6 Hz, 1H), 7.00 (s, 2H), 4.29 (dd, J=12.4, 5.2 Hz, 1H), 4.03 (s, 4H), 3.52 (s, 2H), 2.93 (d, J=10.6 Hz, 1H), 2.11 (s, 1H), 2.01 (s, 1H).

### Example 98: Synthesis of 5-chloro-N-[2,4-difluoro-3-[1-(1H-imidazol-2-yl)imidazo[1,5-a]pyrazin-6-yl]phenyl]-2-methylpyridine-3-sulfonamide (Compound 83)

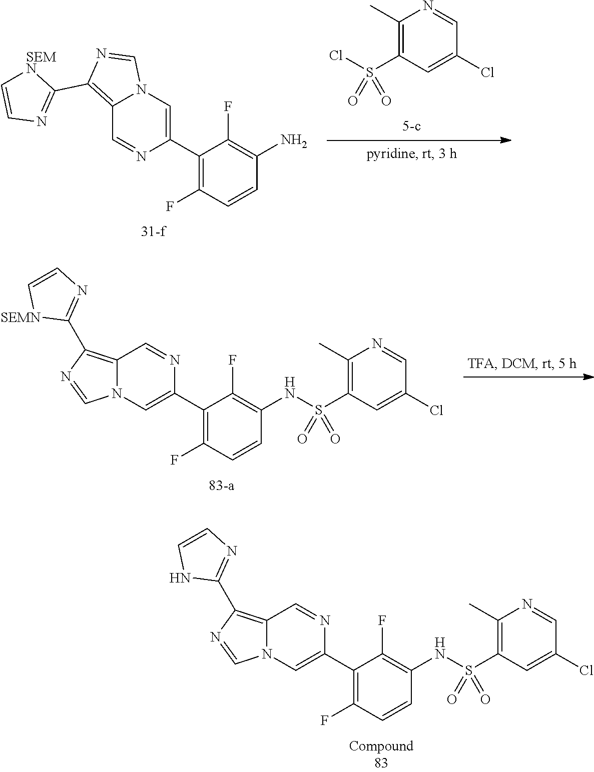 [SMILES: C[Si](C)(C)CCOCn1ccnc1-c1ncn2cc(-c3c(F)ccc(N)c3F)ncc12 ; Cc1ncc(Cl)cc1S(=O)(=O)Cl ; Cc1ncc(Cl)cc1S(=O)(=O)Nc1ccc(F)c(-c2cn3cnc(-c4nccn4COCC[Si](C)(C)C)c3cn2)c1F ; Cc1ncc(Cl)cc1S(=O)(=O)Nc1ccc(F)c(-c2cn3cnc(-c4ncc[nH]4)c3cn2)c1F]

### Synthesis of 83-a: N-[2,4-difluoro-3-[1-(1-[[2-(trimethylsilyl)ethoxy]methyl]imidazol-2-yl)imidazo[1,5-a]pyrazin-6-yl]phenyl]-5-fluoro-2-methoxypyridine-3-sulfonamide

To a stirred solution of 2,4-difluoro-3-[1-(1-[[2-(trimethylsilyl)ethoxy]methyl]imidazol-2-yl)imidazo[1,5-a]pyrazin-6-yl]aniline (120 mg, 0.3 mmol, 1 equiv) in pyridine (3 mL) was added 5-chloro-2-methylpyridine-3-sulfonyl chloride (92 mg, 0.4 mmol, 1.5 equiv) in portions at room temperature. The resulting solution was stirred for 3 h at room temperature, then concentrated under reduced pressure. The residue was purified by prep-HPLC with the following conditions: Column, WelFlash™ C18-I, Spherical C18 20-40 m, 120 g; mobile phase: 5-70% MeCN/0.1% aqueous formic acid; Detector, 220 nm; to give 5-chloro-N-[2,4-difluoro-3-[1-(1-[[2-(trimethylsilyl) ethoxy]methyl]imidazol-2-yl)imidazo[1,5-a]pyrazin-6-yl]phenyl]-2-methylpyridine-3-sulfonamide (85 mg, 49% yield) as a yellow solid.

LCMS (ES, m/z): [M+H]⁺: 632

### Synthesis of Compound 83: 5-chloro-N-[2,4-difluoro-3-[1-(1H-imidazol-2-yl)imidazo[1,5-a]pyrazin-6-yl]phenyl]-2-methylpyridine-3-sulfonamide

Into a 50 mL round-bottom flask, was placed 5-chloro-N-[2,4-difluoro-3-[1-(1-[[2-(trimethylsilyl)ethoxy]methyl]imidazol-2-yl)imidazo[1,5-a]pyrazin-6-yl]phenyl]-2-methylpyridine-3-sulfonamide (85 mg), DCM (6 mL) and TFA (2 mL). The resulting solution was stirred for 5 h then concentrated. The residue was dissolved in 3 mL of MeOH and the pH was adjusted to 8 with NH₃ (7 M in MeOH). This was purified by prep-HPLC with the following conditions: Column, welch Vltimate XB-C18, 50×250 mm, 10 μm; Mobile Phase: 10-50% MeCN/0.1% aqueous formic acid; Detector, 220 nm; to give 5-chloro-N-[2,4-difluoro-3-[1-(1H-imidazol-2-yl)imidazo[1,5-a]pyrazin-6-yl]phenyl]-2-methylpyridine-3-sulfonamide (25 mg, 37% yield) as a yellow solid.

LCMS (ES, m/z): [M+H]⁺: 502

¹H NMR (300 MHz, DMSO-d₆) δ 9.54 (d, J=1.6 Hz, 1H), 8.76 (d, J=2.4 Hz, 1H), 8.67 (s, 1H), 8.55 (d, J=1.5 Hz, 1H), 8.08 (d, J=2.4 Hz, 1H), 7.40 (td, J=8.9, 5.8 Hz, 1H), 7.31-7.11 (m, 3H), 2.77 (s, 3H).

### Example 99: 5-chloro-N-[2,4-difluoro-3-[(6R)-1-(1H-imidazol-2-yl)-5H,6H,7H,8H-imidazo[1,5-a]pyridin-6-yl]phenyl]-2-methylpyridine-3-sulfonamide and 5-chloro-N-[2,4-difluoro-3-[(6S)-1-(1H-imidazol-2-yl)-5H,6H,7H,8H-imidazo[1,5-a]pyridin-6-yl]phenyl]-2-methylpyridine-3-sulfonamide (Compound 84-1 & 84-2)

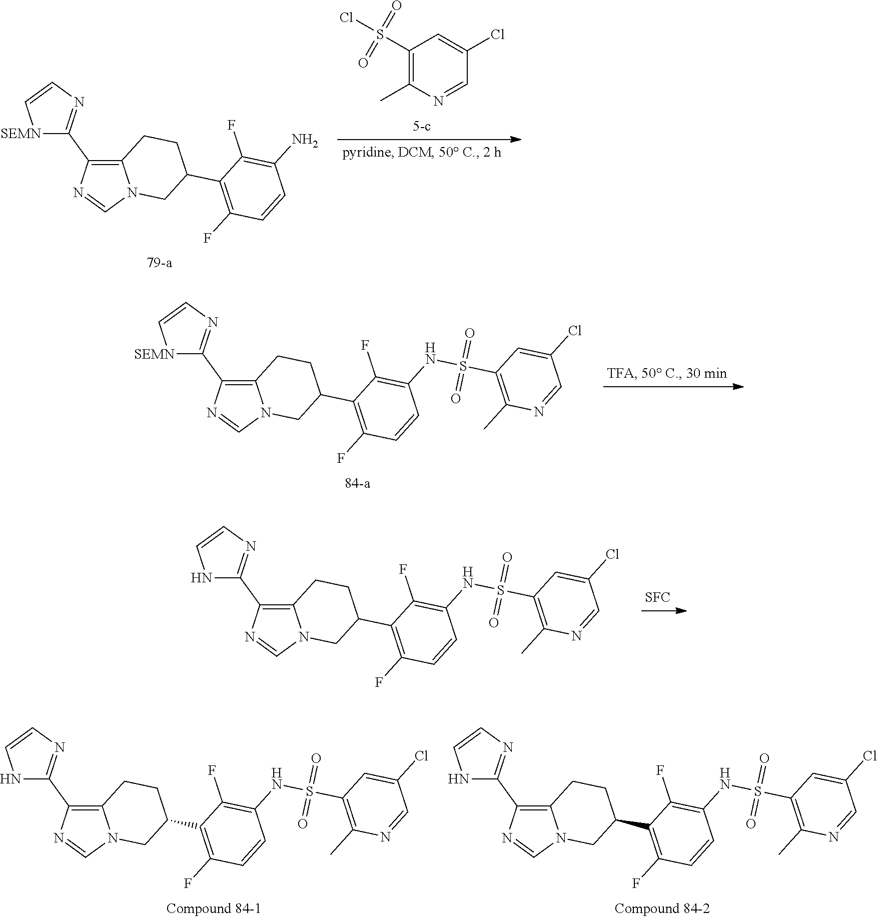

### Synthesis of 84-a: 5-chloro-N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-2-methylpyridine-3-sulfonamide

Into a 25 mL 3-necked round-bottom flask, was placed 2,4-difluoro-3-[1-(1-[[2-(trimethylsilyl)ethoxy]methyl]imidazol-2-yl)-5H,6H,7H,8H-imidazo[1,5-a]pyridin-6-yl]aniline (400 mg, 0.9 mmol, 1 equiv), 5-chloro-2-methylpyridine-3-sulfonyl chloride (203 mg, 0.9 mmol, 1 equiv) and pyridine (213 mg, 2.7 mmol, 3 equiv) in DCM (10 mL). The resulting solution was stirred for 2 h at 45° C., then was cooled and quenched by the addition of 2 mL of CH₃OH. The resulting mixture was concentrated under vacuum and the crude product was purified by Flash-Prep-HPLC with the following conditions: Column, Sunfire Prep C18 OBD, 50*250 mm, 5 m; mobile phase: 10-55% MeCN/0.1% aqueous formic acid; Detector, 220 nm; to give 5-chloro-N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-2-methylpyridine-3-sulfonamide (200 mg, 32% yield) as a yellow solid.

LCMS (ES, m/z): [M+H]⁺: 635

### Synthesis of Compounds 84-1 & 84-2: 5-chloro-N-[2,4-difluoro-3-[(6R)-1-(1H-imidazol-2-yl)-5H,6H,7H,8H-imidazo[1,5-a]pyridin-6-yl]phenyl]-2-methylpyridine-3-sulfonamide and 5-chloro-N-[2,4-difluoro-3-[(6S)-1-(1H-imidazol-2-yl)-5H,6H,7H,8H-imidazo[1,5-a]pyridin-6-yl]phenyl]-2-methylpyridine-3-sulfonamide

Into a 25 mL 3-necked round-bottom flask, was placed 5-chloro-N-[2,4-difluoro-3-[1-(1-[[2-(trimethylsilyl)ethoxy]methyl]imidazol-2-yl)-5H,6H,7H,8H-imidazo[1,5-a]pyridin-6-yl]phenyl]-2-methylpyridine-3-sulfonamide (200 mg, 0.3 mmol, 1 equiv) and TFA (2 mL). The resulting solution was stirred for 30 min at 50° C. then cooled and concentrated under vacuum. The crude product was purified by Flash-Prep-HPLC with the following conditions: Column, Sunfire Prep C18 OBD, 50*250 mm, 5 m; mobile phase: 10-50% MeCN/0.1% aqueous formic acid; Detector 220 nm; to give 5-chloro-N-[2,4-difluoro-3-[1-(1H-imidazol-2-yl)-5H,6H,7H,8H-imidazo[1,5-a]pyridin-6-yl]phenyl]-2-methylpyridine-3-sulfonamide (110 mg, 66% yield) as a white solid.

The enantiomers were separated by SFC with the following conditions: Column, YMC-SC, 30*250 mm, 5 m, Mobile Phase: 50% 1:1 MeOH:EtOH/3:1 Hexane:DCM containing 0.1% diethylamine; Detector, 220 nm. This yielded 5-chloro-N-[2,4-difluoro-3-[(6R)-1-(1H-imidazol-2-yl)-5H,6H,7H,8H-imidazo[1,5-a]pyridin-6-yl]phenyl]-2-methylpyridine-3-sulfonamide (17 mg, 15% yield, stereochemistry randomly assigned) as a white solid and 5-chloro-N-[2,4-difluoro-3-[(6S)-1-(1H-imidazol-2-yl)-5H,6H,7H,8H-imidazo[1,5-a]pyridin-6-yl]phenyl]-2-methylpyridine-3-sulfonamide (18 mg, 16% yield, stereochemistry randomly assigned) as a white solid.

### 5-chloro-N-[2,4-difluoro-3-[(6R)-1-(1H-imidazol-2-yl)-5H,6H,7H,8H-imidazo[1,5-a]pyridin-6-yl]phenyl]-2-methylpyridine-3-sulfonamide

LCMS (ES, m/z): [M+H]⁺: 505

¹H NMR (300 MHz, DMSO-d₆) δ 8.75 (d, J=2.4 Hz, 1H), 7.99 (d, J=2.4 Hz, 1H), 7.61 (s, 1H), 7.24 (td, J=8.9, 5.8 Hz, 1H), 7.09 (t, J=9.7 Hz, 1H), 7.02 (s, 2H), 4.27 (dd, J=12.3, 5.1 Hz, 1H), 3.97 (t, J=11.9 Hz, 1H), 3.54-3.15 (m, 2H), 2.90 (ddd, J=17.5, 11.9, 6.4 Hz, 1H), 2.74 (s, 3H), 2.10-1.90 (m, 2H).

### 5-chloro-N-[2,4-difluoro-3-[(6S)-1-(1H-imidazol-2-yl)-5H,6H,7H,8H-imidazo[1,5-a]pyridin-6-yl]phenyl]-2-methylpyridine-3-sulfonamide

LCMS (ES, m/z): [M+H]⁺: 505

¹H NMR (300 MHz, DMSO-d₆) δ 8.75 (d, J=2.4 Hz, 1H), 7.99 (d, J=2.4 Hz, 1H), 7.61 (s, 1H), 7.24 (td, J=8.9, 5.8 Hz, 1H), 7.16-7.04 (m, 1H), 7.02 (s, 2H), 4.27 (dd, J=12.2, 5.2 Hz, 1H), 3.97 (t, J=11.9 Hz, 1H), 3.60-3.20 (2H, m), 2.90 (ddd, J=17.5, 11.5, 6.4 Hz, 1H), 2.74 (s, 3H), 2.15-1.90 (m, 2H).

### Example 100: Synthesis of 5-chloro-N-[2,4-difluoro-3-[5-(1H-imidazol-2-yl)imidazo[1,5-b]pyridazin-2-yl]phenyl]-2-methoxypyridine-3-sulfonamide (Compound 85)

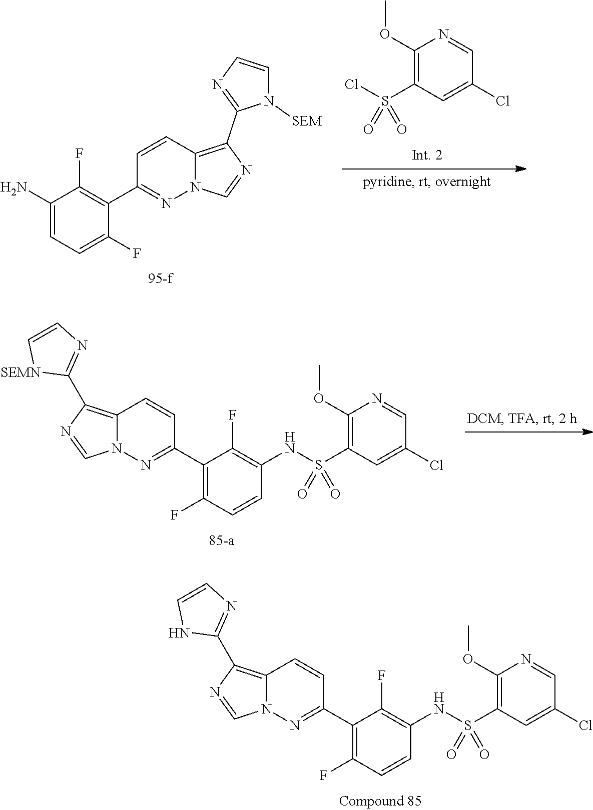 [SMILES: C[Si](C)(C)CCOCn1ccnc1-c1ncn2nc(-c3c(F)ccc(N)c3F)ccc12 ; COc1ncc(Cl)cc1S(=O)(=O)Cl ; COc1ncc(Cl)cc1S(=O)(=O)Nc1ccc(F)c(-c2ccc3c(-c4nccn4COCC[Si](C)(C)C)ncn3n2)c1F ; COc1ncc(Cl)cc1S(=O)(=O)Nc1ccc(F)c(-c2ccc3c(-c4ncc[nH]4)ncn3n2)c1F]

### Synthesis of 85-a: 5-chloro-N-[2,4-difluoro-3-[5-(1-[[2-(trimethylsilyl)ethoxy]methyl]imidazol-2-yl) imidazo[1,5-blpyridazin-2-yl]phenyl]-2-methoxypyridine-3-sulfonamide

To a stirred solution of 2,4-difluoro-3-[5-(1-[[2-(trimethylsilyl)ethoxy]methyl]imidazol-2-yl)imidazo[1,5-b]pyridazin-2-yl]aniline (130 mg, 0.3 mmol, 1 equiv) in pyridine (3 mL) was added 5-chloro-2-methoxypyridine-3-sulfonyl chloride (106 mg, 0.4 mmol, 1.5 equiv) in portions at room temperature. The resulting mixture was stirred overnight at room temperature, then concentrated under reduced pressure. The residue was purified by Flash-Prep-HPLC with the following conditions: Column, WelFlash™ C18-I, Spherical C18 20-40 m, 120 g; mobile phase: 5-70% MeCN/0.1% aqueous formic acid; Detector, 220 nm; to give 5-chloro-N-[2,4-difluoro-3-[5-(1-[[2-(trimethylsilyl)ethoxy]methyl]imidazol-2-yl)imidazo[1,5-b]pyridazin-2-yl]phenyl]-2-methoxypyridine-3-sulfonamide (95 mg, 50% yield) as a yellow solid.

LCMS (ES, m/z): [M+H]⁺: 648

### Synthesis of Compound 85: 5-chloro-N-[2,4-difluoro-3-[5-(1H-imidazol-2-yl)imidazo[1,5-b]pyridazin-2-yl]phenyl]-2-methoxypyridine-3-sulfonamide

Into a 50 mL round-bottom flask, was placed 5-chloro-N-[2,4-difluoro-3-[5-(1-[[2-(trimethylsilyl)ethoxy]methyl]imidazol-2-yl)imidazo[1,5-b]pyridazin-2-yl]phenyl]-2-methoxypyridine-3-sulfonamide (95 mg, 0.15 mmol, 1 equiv), DCM (6 mL) and TFA (2 mL). The resulting solution was stirred for 2 h, then was concentrated. The residue was dissolved in 3 mL of MeOH and the pH adjusted to 8 with NH₃ (7 M in MeOH). This was purified by prep-HPLC with the following conditions: Column, welch Vltimate XB-C18, 50×250 mm, 10 μm; Mobile Phase: 10-40% MeCN/0.1% aqueous formic acid; Detector, 220 nm. This gave 5-chloro-N-[2,4-difluoro-3-[5-(1H-imidazol-2-yl)imidazo[1,5-b]pyridazin-2-yl]phenyl]-2-methoxypyridine-3-sulfonamide (40 mg, 53% yield) as a yellow solid.

LCMS (ES, m/z): [M+H]⁺ 518

H NMR (300 MHz, DMSO-d₆) δ 8.89 (s, 1H), 8.66 (d, J=9.4 Hz, 1H), 8.52 (d, J=2.6 Hz, 1H), 8.10 (d, J=2.6 Hz, 1H), 7.50 (td, J=8.9, 5.8 Hz, 1H), 7.38-7.24 (m, 1H), 7.14 (s, 2H), 6.96 (d, J=9.4 Hz, 1H), 3.93 (s, 3H).

### Example 101: Synthesis of (R)-6-(2,6-difluoro-3-((5-fluoro-2-methylpyridine)-3-sulfonamido)phenyl)-N-methyl-5,6,7,8-tetrahydroimidazo[1,5-a]pyridine-1-carboxamide and (S)-6-(2,6-difluoro-3-((5-fluoro-2-methylpyridine)-3-sulfonamido)phenyl)-N-methyl-5,6,7,8-tetrahydroimidazo[1,5-a]pyridine-1-carboxamide (Compounds 86-1 & 86-2)

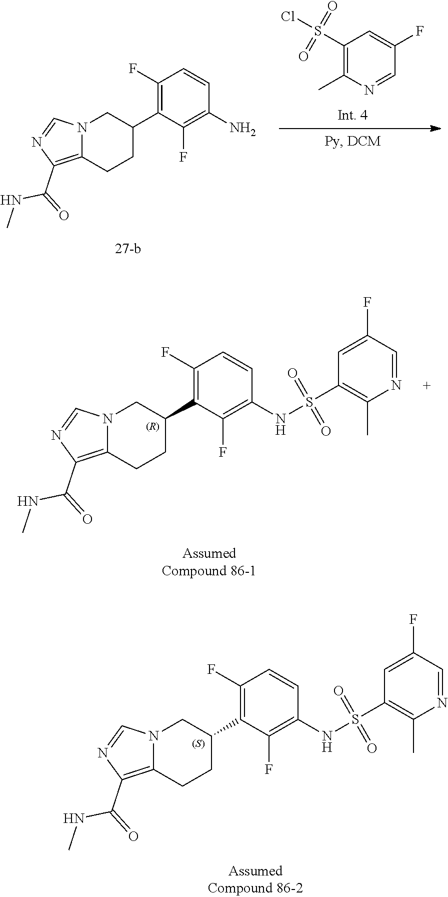

### Synthesis of Compounds 86-1 & 86-2: (R)-6-(2,6-difluoro-3-((5-fluoro-2-methylpyridine)-3-sulfonamido)phenyl)-N-methyl-5,6,7,8-tetrahydroimidazo[1,5-a]pyridine-1-carboxamide and (S)-6-(2,6-difluoro-3-((5-fluoro-2-methylpyridine)-3-sulfonamido)phenyl)-N-methyl-5,6,7,8-tetrahydroimidazo[1,5-a]pyridine-1-carboxamide

To a stirred solution of 6-(3-amino-2,6-difluorophenyl)-N-methyl-5H,6H,7H,8H-imidazo[1,5-a]pyridine-1-carboxamide (200 mg, 0.65 mmol, 1 equiv) in pyridine (10 mL) was added a solution of 5-fluoro-2-methylpyridine-3-sulfonyl chloride (205 mg, 0.98 mmol, 1.5 equiv) in DCM (2 mL) dropwise at room temperature. The resulting solution was stirred for 1 h then quenched with water (20 mL). This was extracted with EA (3×20 mL), and the combined organics were washed with brine (2×10 mL), dried over anhydrous Na₂SO₄ and concentrated under reduced pressure. The residue was purified by prep-HPLC with the following conditions: Column, Sunfire Prep C18 OBD, 50*250 mm 5 μm 10 nm; Mobile Phase: 10-40% MeCN/0.1% aqueous formic acid, Flow rate: 90 mL/min; Detector 220 nm; to give racemic 6-(2,6-difluoro-3-((5-fluoro-2-methylpyridine)-3-sulfonamido)phenyl)-N-methyl-5,6,7,8-tetrahydroimidazo[1,5-a]pyridine-1-carboxamide. The enantiomers were separated by chiral-prep-HPLC with the following conditions: Column, CHIRALPAK IE 250*30 mm, 5 m; Mobile Phase 10% EtOH/5:1 Hexane:DCM; to afford (6R)-6-[2,6-difluoro-3-(5-fluoro-2-methylpyridine-3-sulfonamido)phenyl]-N-methyl-5H,6H,7H,8H-imidazo[1,5-a]pyridine-1-carboxamide (52 mg, 17% yield, stereochemistry randomly assigned) as a white solid and (6S)-6-[2,6-difluoro-3-(5-fluoro-2-methylpyridine-3-sulfonamido)phenyl]-N-methyl-5H,6H,7H,8H-imidazo[1,5-a]pyridine-1-carboxamide (61 mg, 19% yield, stereochemistry randomly assigned) as a white solid.

### (R)-6-(2,6-difluoro-3-((5-fluoro-2-methylpyridine)-3-sulfonamido)phenyl)-N-methyl-5,6,7,8-tetrahydroimidazo[1,5-a]pyridine-1-carboxamide

LCMS (ES, m/z): [M+H]⁺: 480

¹H NMR (300 MHz, DMSO-d₆) δ 10.64 (s, 1H), 8.75 (d, J=2.8 Hz, 1H), 7.87 (dd, J=8.2, 2.9 Hz, 1H), 7.73 (q, J=4.7 Hz, 1H), 7.53 (s, 1H), 7.25 (td, J=8.8, 5.7 Hz, 1H), 7.09 (t, J=9.4 Hz, 1H), 4.23 (dd, J=12.4, 5.2 Hz, 1H), 3.93 (t, J=11.9 Hz, 1H), 3.54-3.42 (m, 1H), 3.37-3.24 (m, 1H), 2.88 (d, J=11.8 Hz, 1H), 2.74 (d, J=1.2 Hz, 3H), 2.71 (d, J=4.8 Hz, 3H), 1.98 (d, J=28.3 Hz, 2H).

### (S)-6-(2,6-difluoro-3-((5-fluoro-2-methylpyridine)-3-sulfonamido)phenyl)-N-methyl-5,6,7,8-tetrahydroimidazo[1,5-a]pyridine-1-carboxamide

LCMS (ES, m/z): [M+H]⁺: 480

¹H NMR (300 MHz, DMSO-d₆) δ 10.63 (s, 1H), 8.71 (d, J=2.8 Hz, 1H), 7.87 (dd, J=8.3, 2.8 Hz, 1H), 7.73 (q, J=4.8 Hz, 1H), 7.53 (s, 1H), 7.22 (td, J=8.9, 5.7 Hz, 1H), 7.06 (t, J=9.6 Hz, 1H), 4.23 (dd, J=12.3, 5.2 Hz, 1H), 3.94 (t, J=11.9 Hz, 1H), 3.52-3.42 (m, 1H), 3.34-3.28 (m, 1H), 2.85 (ddd, J=17.8, 11.7, 6.3 Hz, 1H), 2.74 (d, J=1.2 Hz, 3H), 2.71 (d, J=4.7 Hz, 3H), 1.97 (d, J=17.4 Hz, 2H).

### Example 102: Synthesis of 5-chloro-N-[3-[1-(5-cyclopropyl-4H-1,2,4-triazol-3-yl)imidazo[1,5-a]pyridin-6-yl]-2,4-difluorophenyl]-2-methoxypyridine-3-sulfonamide (Compound 87)

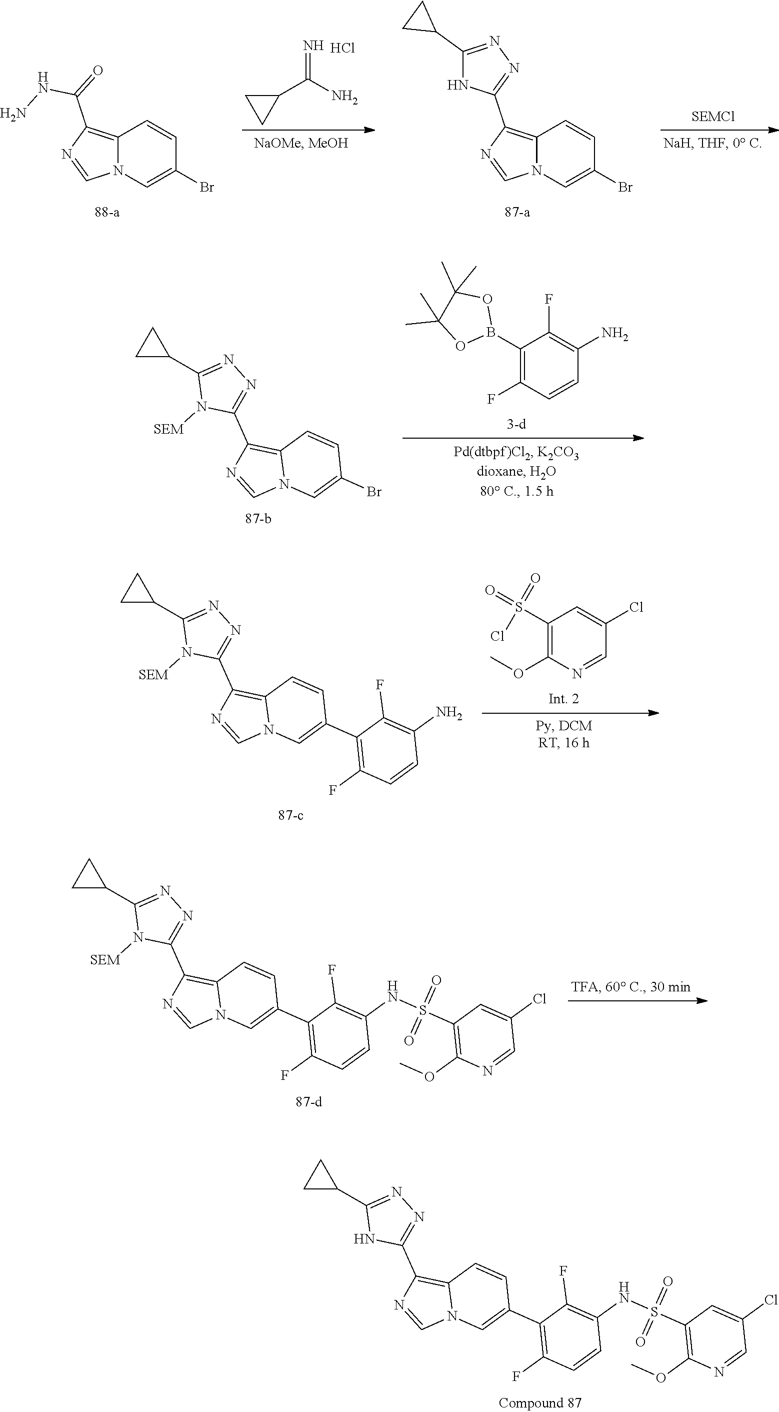

### Synthesis of 87-a: 3-[6-bromoimidazo[1,5-a]pyridin-1-yl]-5-cyclopropyl-4H-1,2,4-triazole

Into a 40 mL vial were added cyclopropaneamidine hydrochloride (1.4 g, 11.6 mmol, 6 equiv), MeOH (20 mL) and 30% NaOMe in MeOH (2.1 g, 11.7 mmol, 6 equiv). The resulting mixture was stirred for 1 h at room temperature. The mixture was filtered, and to the filtrate was added 6-bromoimidazo[1,5-a]pyridine-1-carbohydrazide (500 mg, 2 mmol, 1 equiv). The resulting mixture was stirred for 48 h at 80° C., before being cooled and concentrated under reduced pressure. The residue was purified by silica gel column chromatography, eluting with PE/EtOAc (1:1) to afford 3-[6-bromoimidazo[1,5-a]pyridin-1-yl]-5-cyclopropyl-4H-1,2,4-triazole (410 mg, 69% yield) as a light yellow solid.

LCMS (ES, m/z): [M+H]⁺: 304, 306

### Synthesis of 87-b: 3-[6-bromoimidazo[1,5-a]pyridin-1-yl]-5-cyclopropyl-4-[[2-(trimethylsilyl)ethoxyl methyl]-1,2,4-triazole

Into a 40 mL vial were added 3-[6-bromoimidazo[1,5-a]pyridin-1-yl]-5-cyclopropyl-4H-1,2,4-triazole (340 mg, 1.1 mmol, 1 equiv) and THF (5 mL). To this was added 60% NaH in oil (78 mg, 3.2 mmol, 2.9 equiv) in portions at 0° C. The resulting mixture was stirred for 30 min at 0° C., then[2-(chloromethoxy)ethyl]trimethylsilane (203 mg, 1.2 mmol, 1.1 equiv) was added dropwise at 0° C. After an additional 30 min at 0° C., the reaction was quenched with water (100 mL) and extracted with EtOAc (3×100 mL). The combined organics were washed with brine (3×50 mL), dried over anhydrous Na₂SO₄ and concentrated under reduced pressure. The residue was purified by silica gel column chromatography, eluting with PE/EtOAc (1:1) to afford 3-[6-bromoimidazo[1,5-a]pyridin-1-yl]-5-cyclopropyl-4-[[2-(trimethylsilyl)ethoxy]methyl]-1,2,4-triazole (370 mg, 77% yield) as an off-white solid.

LCMS (ES, m/z): [M+H]⁺: 434, 436

### Synthesis of 87-c: 3-[I-(5-cyclopropyl-4-[[2-(trimethylsilyl)ethoxy]methyl]-1,2,4-triazol-3-yl)imidazo[1,5-a]pyridin-6-yl]-2,4-difluoroaniline

Into a 40 mL vial were added 3-[6-bromoimidazo[1,5-a]pyridin-1-yl]-5-cyclopropyl-4-[[2-(trimethylsilyl)ethoxy]methyl]-1,2,4-triazole (360 mg, 0.8 mmol, 1 equiv), 2,4-difluoro-3-(4,4,5,5-tetramethyl-1,3,2-dioxaborolan-2-yl)aniline (422 mg, 1.6 mmol, 2 equiv), Pd(dtbpf)Cl₂ (54 mg, 0.08 mmol, 0.1 equiv), K₂CO₃ (343 mg, 2.5 mmol, 3 equiv), dioxane (7.00 mL) and H₂O (1.4 mL). The resulting mixture was stirred for 16 h at 80° C. under nitrogen atmosphere. The mixture was cooled and diluted with water (100 mL), then extracted with EtOAc (3×100 mL). The combined organics were washed with brine (3×50 mL), dried over anhydrous Na₂SO₄ and concentrated under reduced pressure. The residue was purified by silica gel column chromatography, eluting with PE/THF (1:1) to afford 3-[1-(5-cyclopropyl-4-[[2-(trimethylsilyl)ethoxy]methyl]-1,2,4-triazol-3-yl)imidazo[1,5-a]pyridin-6-yl]-2,4-difluoroaniline (350 mg, 88% yield) as a light yellow solid.

LCMS (ES, m/z): [M+H]⁺: 483

### Synthesis of 87-d: 5-chloro-N-[3-[1-(5-cyclopropyl-4-[[2-(trimethylsilyl)ethoxy]methyl]-1,2,4-triazol-3-yl) imidazo[1,5-a]pyridin-6-yl]-2,4-difluorophenyl]-2-methoxypyridine-3-sulfonamide

Into an 8 mL vial were added 3-[1-(5-cyclopropyl-4-[[2-(trimethylsilyl)ethoxy]methyl]-1,2,4-triazol-3-yl)imidazo[1,5-a]pyridin-6-yl]-2,4-difluoroaniline (150 mg, 0.3 mmol, 1 equiv), 5-chloro-2-methoxypyridine-3-sulfonyl chloride (105 mg, 0.43 mmol, 1.4 equiv), DCM (3 mL) and pyridine (120 mg, 1.5 mmol, 4.9 equiv). The resulting mixture was stirred overnight then concentrated under vacuum. The residue was purified by silica gel column chromatography, eluting with PE/THF (10:7) to afford 5-chloro-N-[3-[1-(5-cyclopropyl-4-[[2-(trimethylsilyl)ethoxy]methyl]-1,2,4-triazol-3-yl)imidazo[1,5-a]pyridin-6-yl]-2,4-difluorophenyl]-2-methoxypyridine-3-sulfonamide (160 mg, 75% yield) as a light yellow solid.

LCMS (ES, m/z): [M+H]⁺: 688.

### Synthesis of Compound 87: 5-chloro-N-[3-[1-(5-cyclopropyl-4H-1,2,4-triazol-3-yl)imidazo[1,5-a]pyridin-6-yl]-2,4-difluorophenyl]-2-methoxypyridine-3-sulfonamide

Into an 8 mL vial were added 5-chloro-N-[3-[1-(5-cyclopropyl-4-[[2-(trimethylsilyl)ethoxy]methyl]-1,2,4-triazol-3-yl) imidazo[1,5-a]pyridin-6-yl]-2,4-difluorophenyl]-2-methoxypyridine-3-sulfonamide (150 mg, 0.2 mmol, 1 equiv) and TFA (3 mL). The resulting mixture was stirred for 30 min at 60° C., then concentrated under vacuum. The crude product was purified by Prep-HPLC with the following conditions: Column: Atlantis HILIC OBD, 19*150 mm*5 μm; Mobile Phase: 5-30% MeCN/0.1% aqueous formic acid; Flow rate: 90 mL/min; detector 220 nm; to afford 5-chloro-N-[3-[1-(5-cyclopropyl-4H-1,2,4-triazol-3-yl)imidazo[1,5-a]pyridin-6-yl]-2,4-difluorophenyl]-2-methoxypyridine-3-sulfonamide (56 mg, 46% yield) as a light yellow solid.

LCMS (ES, m/z): [M+H]⁺: 558.

¹H NMR (300 MHz, DMSO-d₆) δ 13.88-13.47 (m, 1H), 10.45 (s, 1H), 8.71-8.45 (m, 2H), 8.17 (d, J=9.5 Hz, 1H), 8.09 (d, J=2.6 Hz, 1H), 7.40 (dd, J=9.0, 5.8 Hz, 1H), 7.33-7.18 (m, 1H), 7.09-6.77 (m, 1H), 3.93 (s, 3H), 2.19-1.99 (m, 1H), 1.20-0.74 (m, 3H).

### Example 103: Synthesis of 5-chloro-N-[2,4-difluoro-3-[1-(5-isopropyl-4H-1,24-triazol-3-yl)imidazo[1,5-a]pyridin-6-yl]phenyl]-2-methoxypyridine-3-sulfonamide (Compound 88)

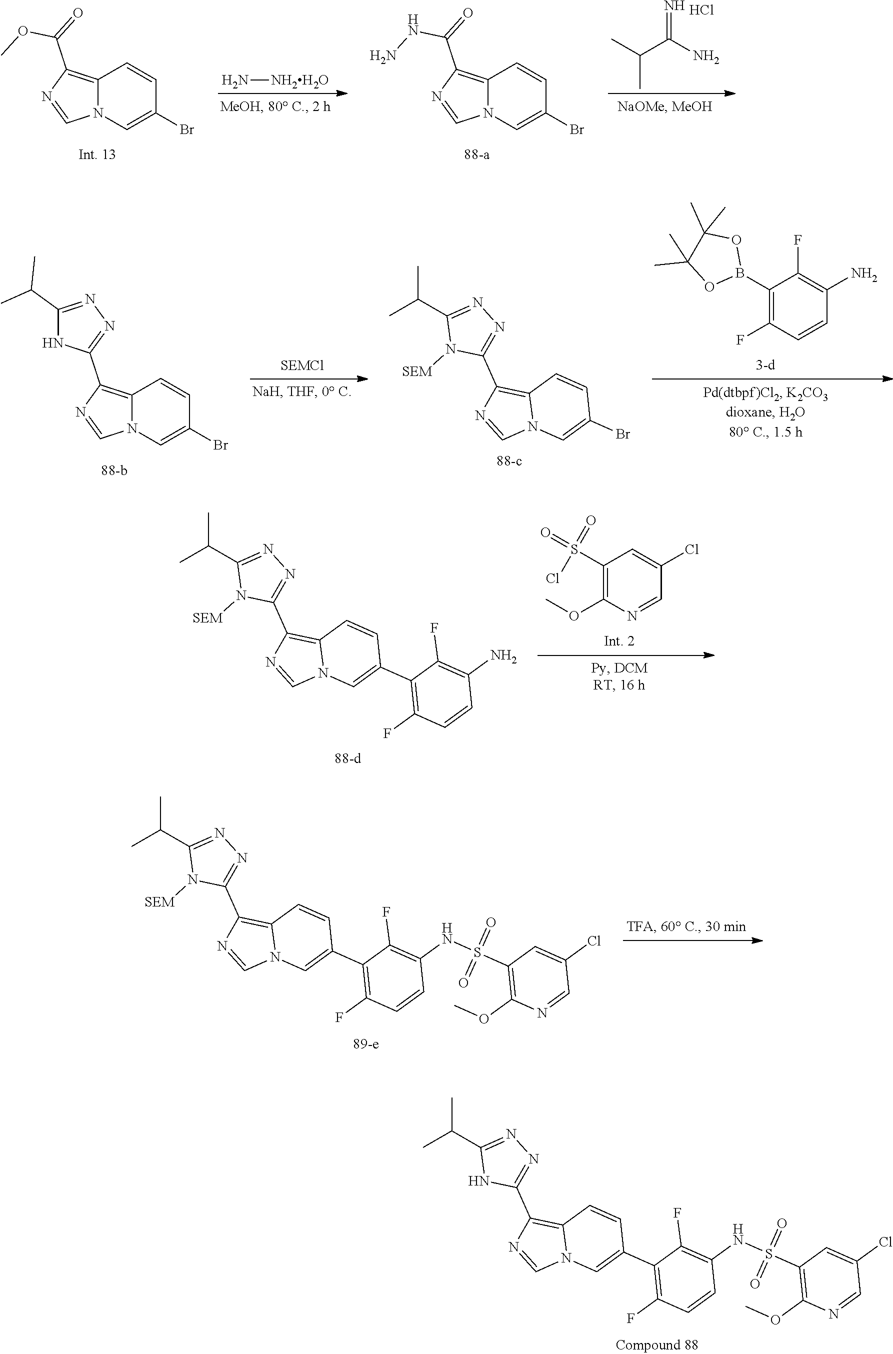

### Synthesis of 88-a: 6-bromoimidazo[1,5-a]pyridine-1-carbohydrazide

Into a 100 mL round-bottom flask were added ethyl 6-bromoimidazo[1,5-a]pyridine-1-carboxylate (3 g, 11 mmol, 1 equiv), hydrazine hydrate (30 mL) and EtOH (30 mL). The resulting mixture was stirred for 2 h at 80° C., then cooled and diluted with water (200 mL). The precipitated solids were collected by filtration, washed with water (2×10 mL) and dried to give 6-bromoimidazo[1,5-a]pyridine-1-carbohydrazide (320 mg, 68% yield) as an off-white solid.

LCMS (ES, m/z): [M+H]⁺: 255, 257

### Synthesis of 88-b: 3-[6-bromoimidazo[1,5-a]pyridin-1-yl]-5-isopropyl-4H-1,2,4-triazole

Into a 40 mL vial were added 2-methylpropanimidamide hydrochloride (1.4 g, 11.7 mmol, 6 equiv), MeOH (20 mL) and 30% NaOMe in MeOH (2.1 g, 11.7 mmol, 6 equiv). The resulting mixture was stirred for 1 h, then filtered. To the filtrate was added 6-bromoimidazo[1,5-a]pyridine-1-carbohydrazide (500 mg, 2 mmol, 1 equiv) and the resulting mixture was stirred for 48 h at 80° C. After cooling, the reaction was concentrated under reduced pressure, and the residue was purified by silica gel column chromatography, eluting with PE/EtOAc (1:1) to afford 3-[6-bromoimidazo[1,5-a]pyridin-1-yl]-5-isopropyl-4H-1,2,4-triazole (390 mg, 65% yield) as a light yellow solid.

LCMS (ES, m/z): [M+H]⁺: 306, 308.

### Synthesis of 88-c: 3-[6-bromoimidazo[1,5-a]pyridin-1-yl]-5-isopropyl-4-[[2-(trimethylsilyl)ethoxy]methyl]-1,2,4-triazole

Into a 40 mL vial were added 3-[6-bromoimidazo[1,5-a]pyridin-1-yl]-5-isopropyl-4H-1,2,4-triazole (320 mg, 1 mmol, 1 equiv) and THE (5 mL). To the above mixture was added 60% NaH (74 mg, 3 mmol, 3 equiv) in portions at 0° C. The resulting mixture was stirred for 30 min at 0° C. before [2-(chloromethoxy)ethyl]trimethylsilane (191 mg, 1.1 mmol, 1.1 equiv) was added dropwise. The reaction was stirred for 30 min at 0° C., then quenched with water. The resulting mixture was diluted further with water (100 mL), and extracted with EtOAc (3×100 mL). The combined organics were washed with brine (3×50 mL), dried over anhydrous Na₂SO₄ and concentrated under reduced pressure. The residue was purified by silica gel column chromatography, eluting with PE/EtOAc (1:1) to afford 3-[6-bromoimidazo[1,5-a]pyridin-1-yl]-5-isopropyl-4-[[2-(trimethylsilyl)ethoxy]methyl]-1,2,4-triazole (340 mg, 74% yield) as an off-white solid.

LCMS (ES, m/z): [M+H]⁺: 436, 438.

### Synthesis of 88-d: 2,4-difluoro-3-[1-(5-isopropyl-4-[[2-(trimethylsilyl)ethoxy]methyl]-1,2,4-triazol-3-yl) imidazo[1,5-a]pyridin-6-yl]aniline

Into a 40 mL vial were added 3-[6-bromoimidazo[1,5-a]pyridin-1-yl]-5-isopropyl-4-[[2-(trimethylsilyl)ethoxy]methyl]-1,2,4-triazole (330 mg, 0.7 mmol, 1 equiv), 2,4-difluoro-3-(4,4,5,5-tetramethyl-1,3,2-dioxaborolan-2-yl)aniline (385 mg, 1.5 mmol, 2 equiv), Pd(dtbpf)Cl₂ (50 mg, 0.08 mmol, 0.1 equiv), K₂CO₃ (313 mg, 2.3 mmol, 3 equiv), dioxane (7 mL) and H₂O (1.4 mL). The resulting mixture was stirred for 16 h at 80° C. under nitrogen atmosphere. The mixture was allowed to cool and was diluted with water (100 mL). The resulting mixture was extracted with EtOAc (3×100 mL), and the combined organics washed with brine (3×50 mL), dried over anhydrous Na₂SO₄ and concentrated under reduced pressure. The residue was purified by silica gel column chromatography, eluting with PE/THF (1:1) to afford 2,4-difluoro-3-[1-(5-isopropyl-4-[[2-(trimethylsilyl)ethoxy]methyl]-1,2,4-triazol-3-yl)imidazo[1,5-a]pyridin-6-yl]aniline (330 mg, 90% yield) as a light yellow solid.

LCMS (ES, m/z): [M+H]⁺: 485.

### Synthesis of 88-e: 5-chloro-N-[2,4-difluoro-3-[1-(5-isopropyl-4-[[2-(trimethylsilyl)ethoxy]methyl]-1,2,4-triazol-3-yl)imidazo[1,5-a]pyridin-6-yl]phenyl]-2-methoxypyridine-3-sulfonamide

Into an 8 mL vial were added 2,4-difluoro-3-[1-(5-isopropyl-4-[[2-(trimethylsilyl)ethoxy]methyl]-1,2,4-triazol-3-yl)imidazo[1,5-a]pyridin-6-yl]aniline (150 mg, 0.3 mmol, 1 equiv), 5-chloro-2-methoxypyridine-3-sulfonyl chloride (112 mg, 0.5 mmol, 1.5 equiv), DCM (3 mL) and pyridine (122 mg, 1.5 mmol, 5 equiv). The resulting mixture was stirred overnight at room temperature, then concentrated under vacuum. The residue was purified by silica gel column chromatography, eluting with PE/THF (10:7) to afford 5-chloro-N-[2,4-difluoro-3-[1-(5-isopropyl-4-[[2-(trimethylsilyl)ethoxy]methyl]-1,2,4-triazol-3-yl)imidazo[1,5-a]pyridin-6-yl]phenyl]-2-methoxypyridine-3-sulfonamide (170 mg, 80% yield) as a light yellow solid.

LCMS (ES, m/z): [M+H]⁺: 690.

### Synthesis of Compound 88: 5-chloro-N-[2,4-difluoro-3-[1-(5-isopropyl-4H-1,2,4-triazol-3-yl)imidazo[1,5-a]pyridin-6-yl]phenyl]-2-methoxypyridine-3-sulfonamide

Into an 8 mL vial were added 5-chloro-N-[2,4-difluoro-3-[1-(5-isopropyl-4-[[2-(trimethylsilyl)ethoxy]methyl]-1,2,4-triazol-3-yl)imidazo[1,5-a]pyridin-6-yl]phenyl]-2-methoxypyridine-3-sulfonamide (160 mg, 0.23 mmol, 1 equiv) and TFA (3 mL). The resulting mixture was stirred for 30 min at 60° C., then was concentrated under vacuum. The crude product was purified by Prep-HPLC with the following conditions: Column: Atlantis HILIC OBD, 19*150 mm*5 μm; Mobile Phase: 5-30% MeCN/0.1% aqueous formic acid; Flow rate: 90 mL/min; Detector 220 nm; to afford 5-chloro-N-[2,4-difluoro-3-[1-(5-isopropyl-4H-1,2,4-triazol-3-yl)imidazo[1,5-a]pyridin-6-yl]phenyl]-2-methoxy pyridine-3-sulfonamide (82 mg, 63% yield) as a light yellow solid.

LCMS (ES, m/z): [M+H]⁺: 560.

¹H NMR (300 MHz, DMSO-d₆) δ 13.93-13.46 (m, 1H), 10.46 (s, 1H), 8.65-8.41 (m, 3H), 8.22 (d, J=9.5 Hz, 1H), 8.09 (d, J=2.6 Hz, 1H), 7.39 (td, J=8.8, 5.8 Hz, 1H), 7.26 (td, J=9.1, 1.5 Hz, 1H), 7.09-6.85 (m, 1H), 3.93 (s, 3H), 3.20-2.93 (m, 1H), 1.33 (d, J=7.0 Hz, 6H).

### Example 104: Synthesis of (R)-6-(3-((5-chloro-2-methylpyridine)-3-sulfonamido)-2,6-difluorophenyl)-N-methyl-5,6,7,8-tetrahydroimidazo[1,5-a]pyridine-1-carboxamide & (S)-6-(3-((5-chloro-2-methylpyridine)-3-sulfonamido)-2,6-difluorophenyl)-N-methyl-5,6,7,8-tetrahydroimidazo[1,5-a]pyridine-1-carboxamide (Compounds 89-1 & 89-2)

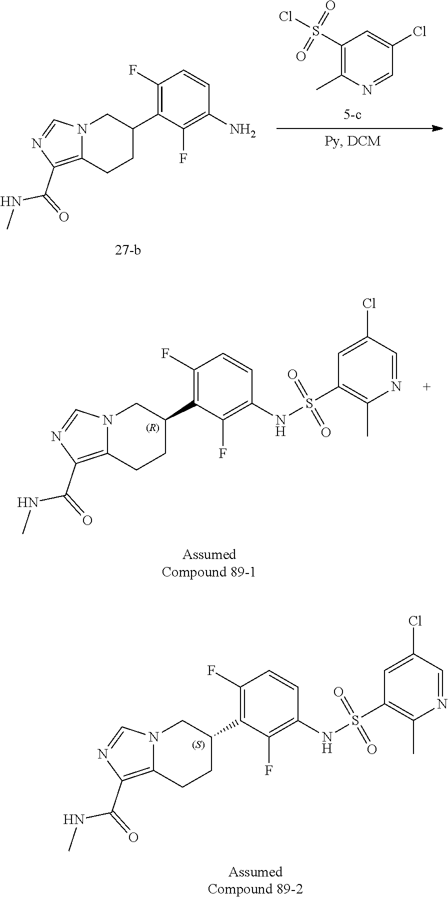

### Synthesis of Compounds 89-1 & 89-2: (R)-6-(3-((5-chloro-2-methylpyridine)-3-sulfonamido)-2,6-difluorophenyl)-N-methyl-5,6,7,8-tetrahydroimidazo[1,5-a]pyridine-1-carboxamide and (S)-6-(3-((5-chloro-2-methylpyridine)-3-sulfonamido)-2,6-difluorophenyl)-N-methyl-5,6,7,8-tetrahydroimidazo[1,5-a]pyridine-1-carboxamide

To a stirred solution of 6-(3-amino-2,6-difluorophenyl)-N-methyl-5H,6H,7H,8H-imidazo[1,5-a]pyridine-1-carboxamide (200 mg, 0.65 mmol, 1 equiv) in pyridine (6 mL) was added a solution of 5-chloro-2-methylpyridine-3-sulfonyl chloride (221 mg, 0.98 mmol, 1.5 equiv) in DCM (1 mL) dropwise at room temperature. The resulting solution was stirred for 1 h then quenched with water (20 mL). The resulting mixture was extracted with EA (3×20 mL), and the combined organics washed with brine (2×10 mL), dried over anhydrous Na₂SO₄ and concentrated under reduced pressure. The residue was purified by prep-HPLC with the following conditions: Column, Sunfire Prep C18 OBD, 50*250 mm 5 μm 10 nm; Mobile Phase: 15-30% MeCN/0.1% aqueous formic acid; to give the racemate of the title compounds. The enantiomers were separated by chiral prep-HPLC using the following conditions: Column, CHIRALPAK IE 250*30 mm, 5 m, Mobile Phase: 30% EtOH/5:1 Hexane:DCM; to afford (6R)-6-[3-(5-chloro-2-methylpyridine-3-sulfonamido)-2,6-difluorophenyl]-N-methyl-5H,6H,7H,8H-imidazo[1,5-a]pyridine-1-carboxamide (50 mg, 15% yield, stereochemistry randomly assigned) as a white solid and (6S)-6-[3-(5-chloro-2-methylpyridine-3-sulfonamido)-2,6-difluorophenyl]-N-methyl-5H,6H,7H,8H-imidazo[1,5-a]pyridine-1-carboxamide (49 mg, 15% yield, stereochemistry randomly assigned) as a white solid.

### (R)-6-(3-((5-chloro-2-methylpyridine)-3-sulfonamido)-2,6-difluorophenyl)-N-methyl-5,6,7,8-tetrahydroimidazo[1,5-a]pyridine-1-carboxamide

LCMS (ES, m/z): [M+H]⁺: 496

¹H NMR (300 MHz, DMSO-d₆) δ 10.65 (s, 1H), 8.78 (d, J=2.4 Hz, 1H), 7.98 (d, J=2.4 Hz, 1H), 7.74 (q, J=4.7 Hz, 1H), 7.53 (s, 1H), 7.27 (td, J=8.8, 5.8 Hz, 1H), 7.13 (td, J=9.5, 9.0, 1.5 Hz, 1H), 4.23 (dd, J=12.3, 5.2 Hz, 1H), 3.93 (t, J=11.9 Hz, 1H), 3.46 (t, J=11.7 Hz, 1H), 3.28 (dd, J=4.7, 2.2 Hz, 1H), 2.86 (ddd, J=17.7, 11.4, 6.5 Hz, 1H), 2.76-2.68 (m, 6H), 1.97 (dd, J=21.9, 7.8 Hz, 2H).

### (S)-6-(3-((5-chloro-2-methylpyridine)-3-sulfonamido)-2,6-difluorophenyl)-N-methyl-5,6,7,8-tetrahydroimidazo[1,5-a]pyridine-1-carboxamide

LCMS (ES, m/z): [M+H]⁺: 496

¹H NMR (300 MHz, DMSO-d₆) δ 10.64 (s, 1H), 8.74 (d, J=2.4 Hz, 1H), 7.99 (d, J=2.4 Hz, 1H), 7.73 (q, J=4.7 Hz, 1H), 7.53 (s, 1H), 7.23 (td, J=8.9, 5.8 Hz, 1H), 7.14-7.01 (m, 1H), 4.23 (dd, J=12.4, 5.2 Hz, 1H), 3.94 (t, J=11.9 Hz, 1H), 3.46 (t, J=11.5 Hz, 1H), 3.38-3.26 (m, 1H), 2.86 (ddd, J=17.8, 11.5, 6.3 Hz, 1H), 2.77-2.67 (m, 6H), 1.98 (dd, J=25.8, 9.4 Hz, 2H).

### Example 105: Synthesis of 6-[2,6-difluoro-3-(5-fluoro-2-methoxypyridine-3-sulfonamido)phenyl]-N-methylimidazo[1,5-a]pyrazine-1-carboxamide (Compound 90)

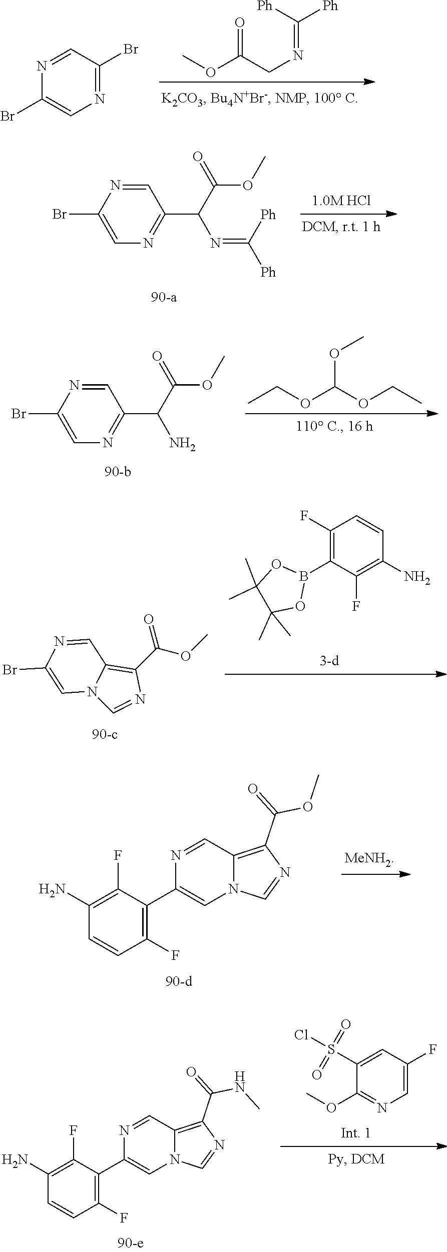

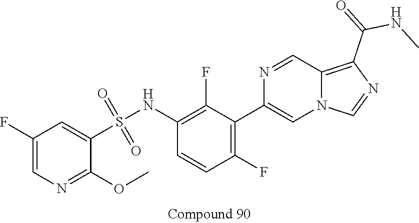 [SMILES: CNC(=O)c1ncn2cc(-c3c(F)ccc(NS(=O)(=O)c4cc(F)cnc4OC)c3F)ncc12]

### Synthesis of 90-a: 2-(5-bromopyrazin-2-yl)-2-|(diphenylmethylidene)amino]acetate

To a stirred mixture of 2,5-dibromopyrazine (10 g, 42 mmol, 1 equiv) and K₂CO₃ (17.4 g, 126 mmol, 3 equiv) in NMP (100 mL) were added methyl 2-[(diphenylmethylidene)amino]acetate (12.8 g, 50 mmol, 1.2 equiv) and tetrabutylammonium bromide (13.6 g, 42 mmol, 1 equiv) in portions at room temperature under nitrogen atmosphere. The reaction was stirred for overnight at 100° C. under nitrogen atmosphere. The resulting mixture was diluted with water (1 L) and extracted with CH₂Cl₂ (3×200 mL). The combined organics were washed with brine (2×200 mL), dried over anhydrous Na₂SO₄ and concentrated under reduced pressure. The residue was purified by silica gel column chromatography, eluting with PE/EtOAc (5:1) to afford methyl 2-(5-bromopyrazin-2-yl)-2-[(diphenylmethylidene)amino]acetate (12 g, 70% yield) as a brown solid.

LCMS (ES, m/z): [M+H]⁺: 410, 412

### Synthesis of 90-b: 2-amino-2-(5-bromopyrazin-2-yl)acetate

To a stirred solution of methyl 2-(5-bromopyrazin-2-yl)-2-[(diphenylmethylidene)amino]acetate (12 g, 29 mmol, 1 equiv) in DCM (300 mL) was added 6 M HCl (20 mL) dropwise at room temperature. The resulting mixture was stirred for 1 h then diluted with water (100 mL). The mixture was basified to pH 10 with ammonia, then extracted with CH₂Cl₂ (3×20 mL). The combined organics were washed with brine (2×20 mL), dried (Na₂SO₄) and concentrated under vacuum. The residue was washed with ethyl ether (3×5 mL) and air dried to give methyl 2-amino-2-(5-bromopyrazin-2-yl)acetate (6 g, 83% yield) as a brown solid.

LCMS (ES, m/z): [M+H]⁺: 246, 248

### Synthesis of 90-c: 6-bromoimidazo[1,5-a]pyrazine-1-carboxylate

Into a 50 mL round-bottom flask were added methyl 2-amino-2-(5-bromopyrazin-2-yl)acetate (6 g, 25 mmol, 1 equiv) and diethoxy(methoxy)methane (10 mL) at room temperature. The resulting mixture was stirred for 16 h at 110° C., then cooled and diluted with diethyl ether (10 mL). The precipitated solids were collected by filtration and washed with diethyl ether (3×5 mL). Drying gave methyl 6-bromoimidazo[1,5-a]pyrazine-1-carboxylate (3 g, 48% yield) as a red solid.

LCMS (ES, m/z): [M+H]⁺: 256, 258

### Synthesis of 90-d: 6-(3-amino-2,6-difluorophenyl)imidazo[1,5-a]pyrazine-1-carboxylate

To a stirred mixture of methyl 6-bromoimidazo[1,5-a]pyrazine-1-carboxylate (500 mg, 2 mmol, 1 equiv) and 2,4-difluoro-3-(4,4,5,5-tetramethyl-1,3,2-dioxaborolan-2-yl)aniline (747 mg, 3 mmol, 1.5 equiv) in 1,4-dioxane (10 mL) were added K₃PO₄ (828 mg, 4 mmol, 2 equiv) and Pd(dtbpf)Cl₂ (127 mg, 0.2 mmol, 0.1 equiv) in portions at room temperature under nitrogen atmosphere. The resulting mixture was stirred for 3 h at 90° C. then concentrated under reduced pressure. The residue was purified by silica gel column chromatography, eluting with PE/EtOAc (10:1) to afford methyl 6-(3-amino-2,6-difluorophenyl)imidazo[1,5-a]pyrazine-1-carboxylate (500 mg) as a yellow solid.

LCMS (ES, m/z): [M+H]⁺: 305

### Synthesis of 90-e: 6-(3-amino-2,6-difluorophenyl)-N-methylimidazo[1,5-a]pyrazine-1-carboxamide

A mixture of methyl 6-(3-amino-2,6-difluorophenyl)imidazo[1,5-a]pyrazine-1-carboxylate (500 mg, 0.25 mmol, 1 equiv) and 40% methylamine aqueous solution (5 mL) in THE (10 mL) was stirred for 1 day at room temperature, then concentrated under reduced pressure. The residue was purified by silica gel column chromatography, eluting with PE/EtOAc (1:10) to afford 6-(3-amino-2,6-difluorophenyl)-N-methylimidazo[1,5-a]pyrazine-1-carboxamide (460 mg) as a white solid.

LCMS (ES, m/z): [M+H]+: 304

### Synthesis of Compound 90: 6-[2,6-difluoro-3-(5-fluoro-2-methoxypyridine-3-sulfonamido)phenyl]-N-methylimidazo[1,5-a]pyrazine-1-carboxamide

To a stirred solution 6-(3-amino-2,6-difluorophenyl)-N-methylimidazo[1,5-a]pyrazine-1-carboxamide (75 mg, 0.25 mmol, 1 equiv) in pyridine (2 mL) was added 5-fluoro-2-methoxypyridine-3-sulfonyl chloride (67 mg, 0.3 mmol, 1.2 equiv) in portions at room temperature. The resulting mixture was stirred for 1 h at room temperature then diluted with MeOH (2 mL) and concentrated. The residue was purified by prep-HPLC with the following conditions: Column, welch Vltimate XB-C18, 50×250 mm, 10 μm; Mobile Phase: 10-50% MeCN/0.1% aqueous formic acid; Detector, 220 nm; to give 6-[2,6-difluoro-3-(5-fluoro-2-methoxypyridine-3-sulfonamido)phenyl]-N-methylimidazo[1,5-a]pyrazine-1-carboxamide (94 mg, 77% yield) as a white solid.

LCMS (ES, m/z): [M+H]+: 493

¹H NMR (300 MHz, DMSO-d₆) δ 10.43 (s, 1H), 9.51 (d, J=1.7 Hz, 1H), 8.67-8.65 (m, 2H), 8.46 (d, J=3.0 Hz, 1H), 8.44-8.41 (m, 1H), 8.03 (dd, J=7.3, 3.0 Hz, 1H) 7.45-7.37 (m, 1H), 7.30-7.18 (m, 1H), 3.91 (s, 3H), 2.84 (d, J=4.8 Hz, 3H).

### Example 106: Synthesis of 6-[2,6-difluoro-3-(5-fluoro-2-methylpyridine-3-sulfonamido)phenyl]-N-methylimidazo[1,5-a]pyrazine-1-carboxamide (Compound 91)

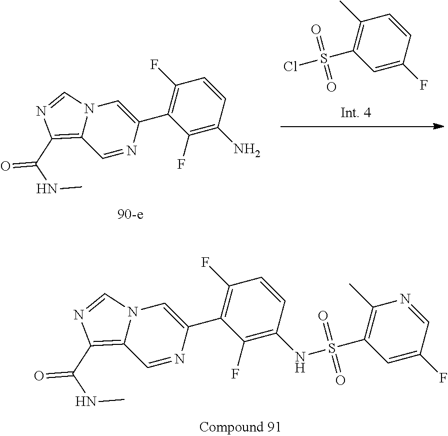 [SMILES: CNC(=O)c1ncn2cc(-c3c(F)ccc(N)c3F)ncc12 ; Cc1ccc(F)cc1S(=O)(=O)Cl ; CNC(=O)c1ncn2cc(-c3c(F)ccc(NS(=O)(=O)c4cc(F)cnc4C)c3F)ncc12]

### Synthesis of Compound 91: 6-[2,6-difluoro-3-(5-fluoro-2-methylpyridine-3-sulfonamido)phenyl]-N-methylimidazo[1,5-a]pyrazine-1-carboxamide

To a stirred solution of 6-(3-amino-2,6-difluorophenyl)-N-methylimidazo[1,5-a]pyrazine-1-carboxamide (115 mg, 0.38 mmol, 1 equiv) in pyridine (2 mL) was added 5-fluoro-2-methylpyridine-3-sulfonyl chloride (238 mg, 1.1 mmol, 3 equiv) dropwise at room temperature. The resulting mixture was stirred for 1 h then concentrated, and the residue was purified by reverse phase flash chromatography with the following conditions: column, C18 silica gel; mobile phase: 10-50% MeOH in water; detector, UV 220 nm; to give 6-[2,6-difluoro-3-(5-fluoro-2-methylpyridine-3-sulfonamido) phenyl]-N-methylimidazo[1,5-a]pyrazine-1-carboxamide (99 mg, 55% yield) as a white solid.

LCMS (ES, m/z): [M+H]+: 477

¹H NMR (300 MHz, DMSO-d₆) δ 10.80 (s, 1H), 9.50 (d, J=1.6 Hz, 1H), 8.72 (d, J=2.8 Hz, 1H), 8.65 (d, J=1.9 Hz, 2H), 8.42 (q, J=4.6 Hz, 1H), 7.95 (dd, J=8.2, 2.8 Hz, 1H), 7.40 (td, J=8.9, 5.8 Hz, 1H), 7.24 (td, J=9.1, 1.6 Hz, 1H), 2.84 (d, J=4.8 Hz, 3H), 2.78 (d, J=1.2 Hz, 3H).

### Example 107: Synthesis of 6-[3-(5-cyano-2-methoxypyridine-3-sulfonamido)-2,6-difluorophenyl]-N-methylimidazo[1,5-a]pyrazine-1-carboxamide (Compound 92)

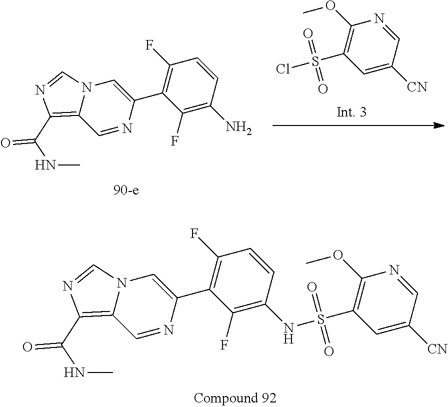 [SMILES: CNC(=O)c1ncn2cc(-c3c(F)ccc(N)c3F)ncc12 ; COc1ncc(C#N)cc1S(=O)(=O)Cl ; CNC(=O)c1ncn2cc(-c3c(F)ccc(NS(=O)(=O)c4cc(C#N)cnc4OC)c3F)ncc12]

### Synthesis of Compound 92: 6-[3-(5-cyano-2-methoxypyridine-3-sulfonamido)-2,6-difluorophenyl]-N-methylimidazo[1,5-a]pyrazine-1-carboxamide

To a stirred solution 6-(3-amino-2,6-difluorophenyl)-N-methylimidazo[1,5-a]pyrazine-1-carboxamide (75 mg, 0.25 mmol, 1 equiv) in pyridine (2 mL) was added 5-cyano-2-methoxypyridine-3-sulfonyl chloride (69 mg, 0.3 mmol, 1.2 equiv) in portions at room temperature. The resulting mixture was stirred for 3 h then diluted with MeOH (2 mL), before being concentrated. The residue was purified by prep-HPLC with the following conditions: Column, welch Vltimate XB-C18, 50×250 mm, 10 μm; Mobile Phase: 10-50% MeCN/0.1% aqueous formic acid; Detector, 220 nm; to afford 6-[3-(5-cyano-2-methoxypyridine-3-sulfonamido)-2,6-difluorophenyl]-N-methylimidazo[1,5-a]pyrazine-1-carboxamide (78 mg, 63% yield) as a white solid.

LCMS (ES, m/z): [M+H]+: 500

¹H NMR (300 MHz, DMSO-d₆) δ 10.54 (s, 1H), 9.51 (d, J=1.6 Hz, 1H), 8.93 (d, J=2.2 Hz, 1H), 8.66 (d, J=2.8 Hz, 2H), 8.51 (d, J=2.2 Hz, 1H), 8.41 (d, J=4.8 Hz, 1H), 7.43 (td, J=8.9, 5.8 Hz, 1H), 7.24 (td, J=9.0, 1.6 Hz, 1H), 4.02 (s, 3H), 2.84 (d, J=4.8 Hz, 3H).

### Example 108: Synthesis of 6-[3-(5-chloro-2-methylpyridine-3-sulfonamido)-2,6-difluorophenyl]-N-methylimidazo[1,5-a]pyrazine-1-carboxamide (Compound 93)

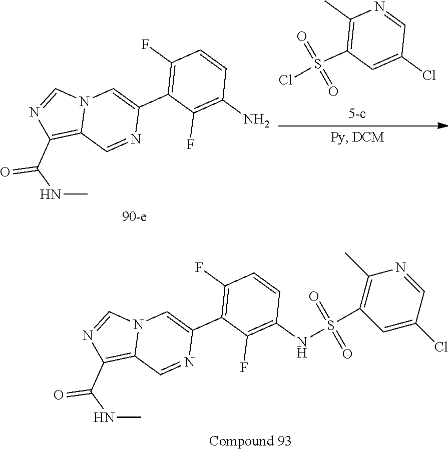 [SMILES: CNC(=O)c1ncn2cc(-c3c(F)ccc(N)c3F)ncc12 ; Cc1ncc(Cl)cc1S(=O)(=O)Cl ; CNC(=O)c1ncn2cc(-c3c(F)ccc(NS(=O)(=O)c4cc(Cl)cnc4C)c3F)ncc12]

### Synthesis of Compound 93: 6-[3-(5-chloro-2-methylpyridine-3-sulfonamido)-2,6-difluorophenyl]-N-methylimidazo[1,5-a]pyrazine-1-carboxamide

To a stirred solution of 6-(3-amino-2,6-difluorophenyl)-N-methylimidazo[1,5-a]pyrazine-1-carboxamide (120 mg, 0.4 mmol, 1 equiv) in pyridine (2 mL) was added 5-chloro-2-methylpyridine-3-sulfonyl chloride (134 mg, 0.6 mmol, 1.5 equiv) dropwise at room temperature. The resulting mixture was stirred for 3 h then diluted with MeOH (2 mL) and concentrated. The residue was purified by prep-HPLC with the following conditions: Column, welch Vltimate XB-C18, 50×250 mm, 10 μm; Mobile Phase: 18-48% MeCN/0.1% aqueous formic acid; Detector, 220 nm; to afford 6-[3-(5-chloro-2-methylpyridine-3-sulfonamido)-2,6-difluorophenyl]-N-methylimidazo[1,5-a]pyrazine-1-carboxamide (79 mg, 40% yield) as a white solid.

LCMS (ES, m/z): [M+H]+: 493

¹H NMR (300 MHz, DMSO-d₆) δ 10.80 (s, 1H), 9.50 (d, J=1.6 Hz, 1H), 8.77 (d, J=2.4 Hz, 1H), 8.65 (s, 2H), 8.41 (t, J=4.8 Hz, 1H), 8.08 (d, J=2.4 Hz, 1H), 7.41 (td, J=8.9, 5.8 Hz, 1H), 7.26 (t, J=9.1 Hz, 1H), 2.84 (d, J=4.8 Hz, 3H), 2.77 (s, 3H).

### Example 109: Synthesis of 5-chloro-N-(2,4-difluoro-3-(1-(hydroxymethyl)imidazo[1,5-a]pyridin-6-yl)phenyl)-2-methoxypyridine-3-sulfonamide (Compound 94)

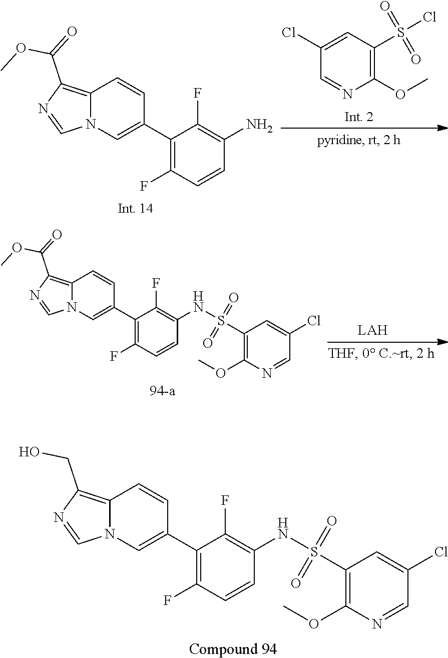 [SMILES: COC(=O)c1ncn2cc(-c3c(F)ccc(N)c3F)ccc12 ; COc1ncc(Cl)cc1S(=O)(=O)Cl ; COC(=O)c1ncn2cc(-c3c(F)ccc(NS(=O)(=O)c4cc(Cl)cnc4OC)c3F)ccc12 ; COc1ncc(Cl)cc1S(=O)(=O)Nc1ccc(F)c(-c2ccc3c(CO)ncn3c2)c1F]

### Synthesis of 94-a: methyl 6-(3-((5-chloro-2-methoxypyridine)-3-sulfonamido)-2,6-difluorophenyl) imidazo[1,5-a]pyridine-1-carboxylate

To a stirred solution of methyl 6-(3-amino-2,6-difluorophenyl)imidazo[1,5-a]pyridine-1-carboxylate (800 mg, 2.64 mmol, 1 equiv) in pyridine (10 mL) was added 5-chloro-2-methoxypyridine-3-sulfonyl chloride (958 mg, 4 mmol, 1.5 equiv) in portions at room temperature. The resulting mixture was stirred for 2 h, then concentrated under reduced pressure. The residue was purified by Flash-Prep-HPLC with the following conditions: Column, WelFlash™ C18-I, Spherical C18 20-40 m, 120 g; mobile phase: 30-80% MeCN/0.1% aqueous formic acid; Detector, 220 nm; to afford methyl 6-[3-(5-chloro-2-methoxypyridine-3-sulfonamido)-2,6-difluorophenyl]imidazo[1,5-a]pyridine-1-carboxylate (810 mg, 60% yield) as a brown oil.

LCMS (ES, m/z): [M+H]⁺: 509

### Synthesis of Compound 94: 5-chloro-N-(2,4-difluoro-3-(1-(hydroxymethyl)imidazo[1,5-a]pyridin-6-yl)phenyl)-2-methoxypyridine-3-sulfonamide

To a stirred solution of methyl 6-[3-(5-chloro-2-methoxypyridine-3-sulfonamido)-2,6-difluorophenyl]imidazo[1,5-a]pyridine-1-carboxylate (200 mg, 0.39 mmol, 1 equiv) in THF (4 mL) was added LAH (23 mg, 0.59 mmol, 1.5 equiv) in portions at 0° C. The resulting mixture was stirred for 2 h at room temperature. The reaction was quenched with H₂O and concentrated under vacuum. The residue was purified by prep-HPLC with the following conditions: Column, welch Vltimate XB-C18, 50×250 mm, 10 μm; Mobile Phase: 35-75% MeCN/0.1% aqueous formic acid; Detector, 220 nm; to afford 5-chloro-N-[2,4-difluoro-3-[1-(hydroxymethyl)imidazo[1,5-a]pyridin-6-yl]phenyl]-2-methoxypyridine-3-sulfonamide (50 mg, 26% yield) as a white solid.

LCMS (ES, m/z): [M+H]⁺: 481

¹H NMR (300 MHz, DMSO-d₆) δ 10.42 (s, 1H), 8.50 (d, J=2.7 Hz, 1H), 8.38 (d, J=14.1 Hz, 2H), 8.08 (d, J=2.7 Hz, 1H), 7.70 (d, J=9.7 Hz, 1H), 7.35 (td, J=9.1, 5.8 Hz, 1H), 7.22 (t, J=9.5 Hz, 1H), 6.62 (d, J=10.4 Hz, 1H), 5.00 (t, J=5.6 Hz, 1H), 4.71 (d, J=5.6 Hz, 2H), 3.92 (s, 3H).

### Example 110: Synthesis of 5-chloro-N-[2,4-difluoro-3-[5-(1H-imidazol-2-yl)imidazo[1,5-b]pyridazin-2-yl]phenyl]-2-methylpyridine-3-sulfonamide (Compound 95)

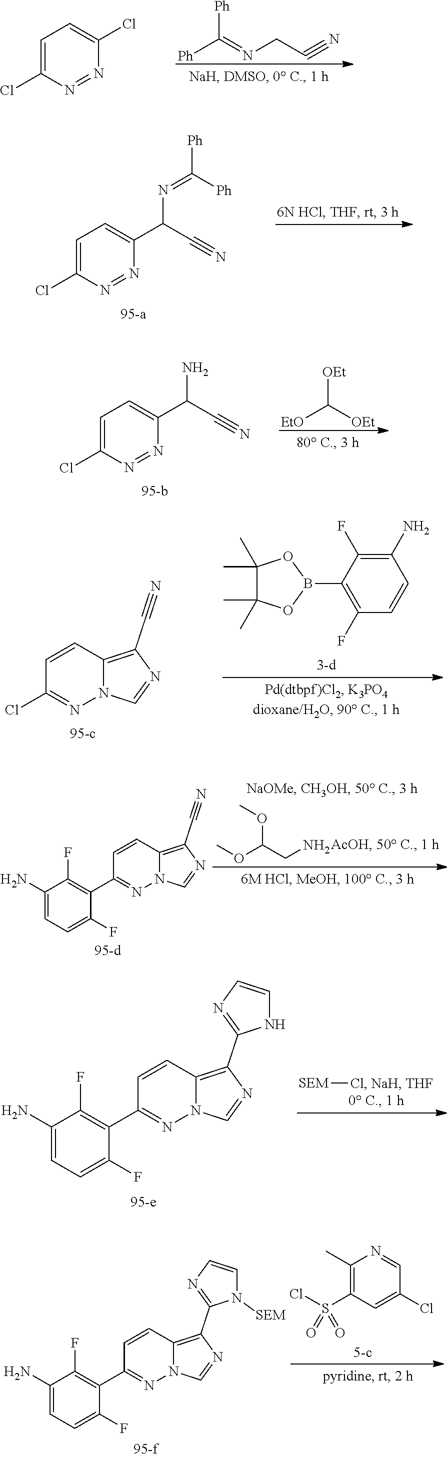

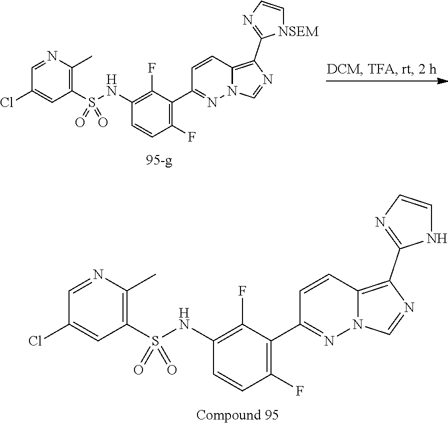 [SMILES: Cc1ncc(Cl)cc1S(=O)(=O)Nc1ccc(F)c(-c2ccc3c(-c4nccn4COCC[Si](C)(C)C)ncn3n2)c1F ; Cc1ncc(Cl)cc1S(=O)(=O)Nc1ccc(F)c(-c2ccc3c(-c4ncc[nH]4)ncn3n2)c1F]

### Synthesis of 95-a: 2-(6-chloropyridazin-3-yl)-2-[(diphenylmethylidene)amino]acetonitrile

To DMSO (340 mL) was added NaH (5.4 g, 134 mmol, 2 equiv, 60% in oil) in portions at 0° C. To this was added 2-[(diphenylmethylidene)amino]acetonitrile (17.7 g, 80 mmol, 1.2 equiv) in DMSO (20 mL) dropwise at 0° C. After 20 min, 3,6-dichloropyridazine (10 g, 67 mmol, 1 equiv) in DMSO (20 mL) was added at 0° C. and the resulting mixture was stirred in an ice bath for 1 hr. The reaction was quenched with water/ice (500 mL) and extracted with EA (3×500 mL). The combined organics were washed with brine (500 mL), dried over anhydrous Na₂SO₄ and concentrated under reduced pressure. The crude product 2-(6-chloropyridazin-3-yl)-2-[(diphenylmethylidene)amino]acetonitrile was used in the next step directly without further purification.

LCMS (ES, m/z): [M+H]⁺: 333

### Synthesis of 95-b: 2-amino-2-(6-chloropyridazin-3-yl)acetonitrile

To a stirred solution of 2-(6-chloropyridazin-3-yl)-2-[(diphenylmethylidene)amino]acetonitrile (20 g, crude) in THE (100 mL) was added 6 M HCl (100 mL). The resulting mixture was stirred for 3 h, then diluted with water (100 mL).

The resulting mixture was extracted with DCM (3×200 mL) and the aqueous layer was basified to pH 8 with ammonia. The resulting mixture was extracted with DCM (3×200 mL). The combined organics were washed with brine (200 mL), dried over anhydrous Na₂SO₄ and concentrated under reduced pressure. This resulted in 2-amino-2-(6-chloropyridazin-3-yl)acetonitrile (8.2 g, 71% yield over the 2 steps) as a brown solid, and which was used in the next step directly without further purification.

LCMS (ES, m/z): [M+H]⁺: 169

### Synthesis of 95-c: 2-chloroimidazo[1,5-b]pyridazine-5-carbonitrile

A solution of 2-amino-2-(6-chloropyridazin-3-yl)acetonitrile (8.2 g, 49 mmol, 1 equiv) in triethyl orthoformate (20 mL) was stirred for 3 h at 80° C. The mixture was allowed to cool and was filtered, the filter cake being washed with Et₂O (2×10 mL). The filtrate was concentrated under reduced pressure to give 2-chloroimidazo[1,5-b]pyridazine-5-carbonitrile (5.8 g, 67% yield) as a brown solid and which was used in the next step directly without further purification.

LCMS (ES, m/z): [M+H]⁺: 179

### Synthesis of 95-d: 2-(3-amino-2,6-difluorophenyl)imidazo[1,5-b]pyridazine-5-carbonitrile

To a stirred mixture of 2-chloroimidazo[1,5-b]pyridazine-5-carbonitrile (1 g, 5.6 mmol, 1 equiv) and 2,4-difluoro-3-(4,4,5,5-tetramethyl-1,3,2-dioxaborolan-2-yl)aniline (2 g, 8.4 mmol, 1.5 equiv) in dioxane (50 mL) and H₂O (10 mL) was added Pd(dtbpf)Cl₂ (0.4 g, 0.6 mmol, 0.1 equiv) and K₃PO₄ (2.4 g, 11 mmol, 2 equiv) at room temperature under N₂ atmosphere. The resulting solution was stirred for 1 h at 90° C. in an oil bath then cooled and concentrated under vacuum. The crude product was re-crystallized from PE/EA (5/1, 20 mL) to afford 2-(3-amino-2,6-difluorophenyl)imidazo[1,5-b]pyridazine-5-carbonitrile (1.5 g, 98%) as a yellow solid and which was used in the next step directly without further purification.

LCMS (ES, m/z): [M+H]⁺: 272

### Synthesis of 95-e: 2,4-difluoro-3-[5-(1H-imidazol-2-yl)imidazo[1,5-b]pyridazin-2-yl]aniline

Into a 50 mL round-bottom flask, was placed 2-(3-amino-2,6-difluorophenyl)imidazo[1,5-b]pyridazine-5-carbonitrile (1.5 g, 5.5 mmol, 1 equiv), MeOH (110 mL) and 30% MeONa in MeOH (2.5 g, 13.8 mmol, 2.5 equiv). The resulting solution was stirred for 3 h at 50° C., then cooled to room temperature and 2,2-dimethoxyethanamine (0.9 g, 8.3 mmol, 1.5 equiv) and AcOH (1.2 g, 19 mmol, 3.5 equiv) were added. The resulting solution was stirred for 1 h at 50° C., then cooled to room temperature. 6 M HCl (10 mL) and MeOH (10 mL) were added and the mixture stirred for 3 h at 100° C., before being concentrated. The resulting solution was diluted with 100 mL of water, and the pH adjusted to 8 with 30% NaOH. The solids formed were collected by filtration and dried to give 2,4-difluoro-3-[5-(1H-imidazol-2-yl)imidazo[1,5-b]pyridazin-2-yl]aniline (1.2 g, 69% yield) as a brown solid and which was used in the next step directly without further purification.

LCMS (ES, m/z): [M+H]⁺: 313

### Synthesis of 95-f: 2,4-difluoro-3-[5-(1-[[2-(trimethylsilyl)ethoxy]methyl]imidazol-2-yl)imidazo[1,5-b]pyridazin-2-yl]aniline

To a stirred solution of 2,4-difluoro-3-[5-(1H-imidazol-2-yl)imidazo[1,5-b]pyridazin-2-yl]aniline (170 mg, 0.5 mmol, 1 equiv) in THE (6 mL) was added NaH (0.3 g, 8.3 mmol, 2 equiv, 60%) in portions at 0° C. SEMCI (1 g, 6 mmol, 1.5 equiv) was added dropwise at low temperature, and the resulting mixture stirred for 1 h in an ice bath. The reaction was quenched with water (100 mL), then extracted with EA (3×100 mL). The combined organics were washed with brine (3×100 mL), dried over anhydrous Na₂SO₄ and concentrated under reduced pressure. The residue was purified by silica gel column chromatography, eluting with PE/EA (1/2) to afford 2,4-difluoro-3-[5-(1-[[2-(trimethylsilyl)ethoxy]methyl]imidazol-2-yl)imidazo[1,5-b]pyridazin-2-yl]aniline (1.2 g, 65% yield) as a yellow solid.

LCMS (ES, m/z): [M+H]⁺: 443

### Synthesis of 95-g: 5-chloro-N-[2,4-difluoro-3-[5-(1-[[2-(trimethylsilyl)ethoxy]methyl]imidazol-2-yl) imidazo[1,5-b]pyridazin-2-yl]phenyl]-2-methylpyridine-3-sulfonamide

To a stirred solution of 2,4-difluoro-3-[5-(1-[[2-(trimethylsilyl)ethoxy]methyl]imidazol-2-yl)imidazo[1,5-b]pyridazin-2-yl]aniline (120 mg, 0.3 mmol, 1 equiv) in pyridine (2 mL) was added 5-chloro-2-methylpyridine-3-sulfonyl chloride (230 mg, 0.8 mmol, 3 equiv, 80%) in portions at 0° C. The resulting mixture was stirred for 2 h and concentrated under reduced pressure. The residue was purified by prep-HPLC with the following conditions: Column, WelFlash™ C18-I, Spherical C18 20-40 m, 120 g; mobile phase: 5-70% MeCN/0.1% aqueous formic acid; Detector, 220 nm. This resulted in 5-chloro-N-[2,4-difluoro-3-[5-(1-[[2-(trimethylsilyl)ethoxy]methyl]imidazol-2-yl)imidazo[1,5-b]pyridazin-2-yl]phenyl]-2-methylpyridine-3-sulfonamide (100 mg, 58% yield) as a yellow solid.

LCMS (ES, m/z): [M+H]⁺: 632

### Synthesis of Compound 95: 5-chloro-N-[2,4-difluoro-3-[5-(1H-imidazol-2-yl)imidazo[1,5-b]pyridazin-2-yl]phenyl]-2-methylpyridine-3-sulfonamide

Into a 50 mL round-bottom flask, was placed 5-chloro-N-[2,4-difluoro-3-[5-(1-[[2-(trimethylsilyl)ethoxy]methyl]imidazol-2-yl)imidazo[1,5-b]pyridazin-2-yl]phenyl]-2-methylpyridine-3-sulfonamide (95 mg, 0.15 mmol, 1 equiv), DCM (6 mL) and TFA (2 mL). The resulting solution was stirred for 2 h and concentrated. The residue was dissolved in 3 mL of MeOH and the pH adjusted to 6 with NH₃ in MeOH (7 mol/L). After concentration, the residue was purified by prep-HPLC with the following conditions: Column, welch Vltimate XB-C18, 50×250 mm, 10 μm; Mobile Phase: 5-35% MeCN/0.1% aqueous formic acid; Detector, 220 nm; to afford 5-chloro-N-[2,4-difluoro-3-[5-(1H-imidazol-2-yl)imidazo[1,5-b]pyridazin-2-yl]phenyl]-2-methylpyridine-3-sulfonamide (50 mg, 66% yield) as a yellow solid.

LCMS (ES, m/z): [M+H]⁺: 502

¹H NMR (300 MHz, DMSO-d₆) δ 11.88 (br s, 1H), 8.87 (s, 1H), 8.74 (d, J=2.4 Hz, 1H), 8.69-8.55 (m, 1H), 8.08 (d, J=2.4 Hz, 1H), 7.46 (td, J=9.0, 5.9 Hz, 1H), 7.25 (td, J=9.1, 1.6 Hz, 1H), 7.12 (s, 2H), 6.92 (dt, J=9.4, 1.3 Hz, 1H), 2.78 (s, 3H).

### Example 111: Synthesis of (5S,6R)-6-[3-(5-chloro-2-methoxypyridine-3-sulfonamido)-2,6-difluorophenyl]-N,5-dimethyl-5H,6H,7H,8H-imidazo[1,5-a]pyridine-1-carboxamide and (5R,6S)-6-[3-(5-chloro-2-methoxypyridine-3-sulfonamido)-2,6-difluorophenyl]-N,5-dimethyl-5H,6H,7H,8H-imidazo[1,5-a]pyridine-1-carboxamide (Compounds 96-1 & 96-2)

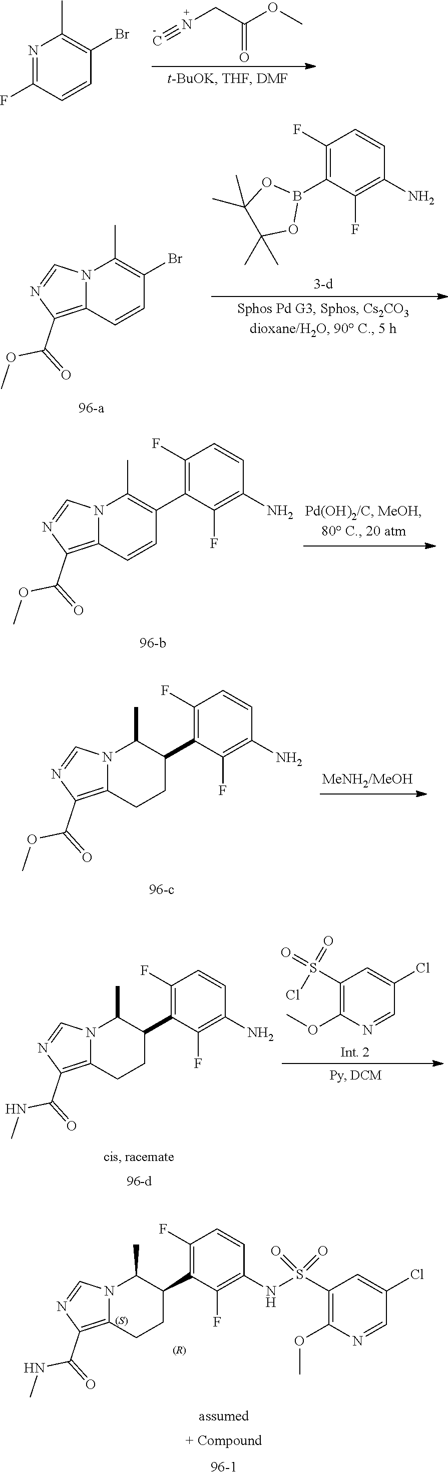

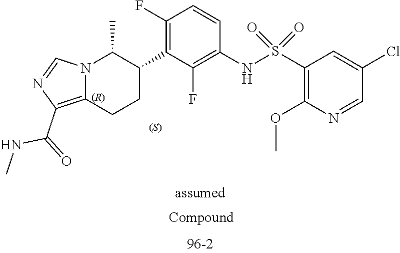

### Synthesis of 96-a: 6-bromo-5-methylimidazo[1,5-a]pyridine-1-carboxylate

To a stirred solution of 3-bromo-6-fluoro-2-methylpyridine (10 g, 52 mmol, 1 equiv) and methyl 2-isocyanoacetate (6.3 g, 63 mmol, 1.2 equiv) in DMF (100 mL) was added t-BuOK (105 mL, 63 mmol, 1.2 equiv, 1 M solution in THF) dropwise at room temperature. The resulting mixture was stirred for 1 h then water (300 mL) was added. The mixture was extracted with ethyl acetate (3×200 mL), and the combined organics were washed with brine (2×50 mL), dried over Na₂SO₄ and concentrated under reduced pressure. The residue was purified by column chromatography over silica gel (eluent: PE:EA=4:1) to give methyl 6-bromo-5-methylimidazo[1,5-a]pyridine-1-carboxylate (5 g, 35% yield) as a white solid.

LCMS (ES, m/z): [M+H]⁺: 269, 271

### Synthesis of 96-b: 6-(3-amino-2,6-difluorophenyl)-5-methylimidazo[1,5-a]pyridine-1-carboxylate

To a solution of methyl 6-bromo-5-methylimidazo[1,5-a]pyridine-1-carboxylate (5 g, 18 mmol, 1 equiv), 2,4-difluoro-3-(4,4,5,5-tetramethyl-1,3,2-dioxaborolan-2-yl)aniline (9.48 g, 37 mmol, 2 equiv) and Cs₂CO₃ (12.1 g, 37 mmol, 2 equiv) in dioxane (50 mL) and H₂O (10 mL) were added SPhos (762 mg, 1.8 mmol, 0.1 equiv) and SPhos Pd G3 (1.4 g, 1.8 mmol, 0.1 equiv) at room temperature under nitrogen atmosphere. After stirring for 5 h at 90° C., the resulting mixture was allowed to cool to room temperature and was diluted with water (50 mL), then extracted with EA (3×100 mL). The combined organics were washed with brine (3×50 mL), dried over anhydrous Na₂SO₄ and concentrated under reduced pressure. The residue was purified by silica gel column chromatography, eluting with PE:EA (2:1) to afford methyl 6-(3-amino-2,6-difluorophenyl)-5-methylimidazo[1,5-a]pyridine-1-carboxylate (1.8 g, 31% yield) as a yellow solid.

LCMS (ES, m/z): [M+H]⁺: 318

### Synthesis of 96-c: cis-methyl 6-(3-amino-2,6-difluorophenyl)-5-methyl-5,6,7,8-tetrahydroimidazo[1,5-a]pyridine-1-carboxylate

To a solution of methyl 6-(3-amino-2,6-difluorophenyl)-5-methylimidazo[1,5-a]pyridine-1-carboxylate (1 g, 3.1 mmol, 1 equiv) in MeOH (50 mL) was added Pd(OH)₂/C (335 mg, 20%) in a pressure tank. The mixture was hydrogenated at 80° C. under 30 atm of hydrogen pressure for 16 h, then filtered through a Celite pad and concentrated under reduced pressure. The residue was purified by Prep-HPLC to afford cis-methyl 6-(3-amino-2,6-difluorophenyl)-5-methyl-5H,6H,7H,8H-imidazo[1,5-a]pyridine-1-carboxylate (150 mg, 15% yield) as a yellow solid.

LCMS (ES, m/z): [M+H]⁺: 322

### Synthesis of 96-d: cis-6-(3-amino-2,6-difluorophenyl)-N,5-dimethyl-5,6,7,8-tetrahydroimidazo[1,5-a]pyridine-1-carboxamide

A mixture of cis-methyl 6-(3-amino-2,6-difluorophenyl)-5-methyl-5H,6H,7H,8H-imidazo[1,5-a]pyridine-1-carboxylate (200 mg) and CH₃NH₂ solution in MeOH (5 mL, 2M) was stirred at 100° C. for 5 h. After being cooled to room temperature, the resulting solution was concentrated under reduced pressure to give cis-6-(3-amino-2,6-difluorophenyl)-N,5-dimethyl-5H,6H,7H,8H-imidazo[1,5-a]pyridine-1-carboxamide (160 mg, 80% yield) as a brown solid.

LCMS (ES, m/z): [M+H]⁺: 321

### Synthesis of Compounds 96-1 & 96-2: (5S,6R)-6-[3-(5-chloro-2-methoxypyridine-3-sulfonamido)-2,6-difluorophenyl]-N,5-dimethyl-5H,6H,7H,8H-imidazo[1,5-a]pyridine-1-carboxamide and (5R,6S)-6-[3-(5-chloro-2-methoxypyridine-3-sulfonamido)-2,6-difluorophenyl]-N,5-dimethyl-5H,6H,7H,8H-imidazo[1,5-a]pyridine-1-carboxamide

To a stirred solution of cis-6-(3-amino-2,6-difluorophenyl)-N,5-dimethyl-5H,6H,7H,8H-imidazo[1,5-a]pyridine-1-carboxamide (150 mg, 0.46 mmol, 1 equiv) and pyridine (111 mg, 1.4 mmol, 3 equiv) in DCM (5 mL) was added 5-chloro-2-methoxypyridine-3-sulfonyl chloride (170 mg, 0.7 mmol, 1.5 equiv) at room temperature. The reaction mixture was stirred for 1 h, then concentrated under reduced pressure to give a residue, which was purified by column chromatography over silica gel (eluent: PE:EA=1:1) and chiral prep-HPLC with the following conditions: Column, CHIRALART, SB, 250×30 mm, 5 m; Mobile Phase: 30% EtOH/Hexane to afford (5S,6R)-6-[3-(5-chloro-2-methoxypyridine-3-sulfonamido)-2,6-difluorophenyl]-N,5-dimethyl-5H,6H,7H,8H-imidazo[1,5-a]pyridine-1-carboxamide (Rt=11 min, 60 mg, 24% yield, cis stereochemistry randomly assigned) as a white solid and (5R,6S)-6-[3-(5-chloro-2-methoxypyridine-3-sulfonamido)-2,6-difluorophenyl]-N,5-dimethyl-5H,6H,7H,8H-imidazo[1,5-a]pyridine-1-carboxamide (Rt=15 mins, 60 mg, 24% yield, opposite cis stereochemistry assigned) as a white solid.

### (5S,6R)-6-[3-(5-chloro-2-methoxypyridine-3-sulfonamido)-2,6-difluorophenyl]-N,5-dimethyl-5H,6H,7H,8H-imidazo[1,5-a]pyridine-1-carboxamide

LCMS (ES, m/z): [M+H]⁺: 526

¹H NMR (300 MHz, DMSO-d₆) δ 10.32 (s, 1H), 8.51 (d, J=2.6 Hz, 1H), 7.95 (d, J=2.6 Hz, 1H), 7.72 (d, J=4.8 Hz, 1H), 7.67 (s, 1H), 7.31 (td, J=8.8, 5.8 Hz, 1H), 7.16-7.04 (m, 1H), 4.48-4.34 (m, 1H), 3.96 (s, 3H), 3.68-3.56 (m, 1H), 3.26 (s, 1H), 2.83 (ddd, J=17.6, 11.6, 6.1 Hz, 1H), 2.71 (d, J=4.7 Hz, 3H), 2.41-2.24 (m, 1H), 1.92 (d, J=13.4 Hz, 1H), 1.00 (d, J=6.6 Hz, 3H).

### (5R,6S)-6-[3-(5-chloro-2-methoxypyridine-3-sulfonamido)-2,6-difluorophenyl]-N,5-dimethyl-5H,6H,7H,8H-imidazo[1,5-a]pyridine-1-carboxamide

LCMS (ES, m/z): [M+H]⁺: 526

¹H NMR (300 MHz, DMSO-d₆) δ 10.32 (s, 1H), 8.51 (d, J=2.6 Hz, 1H), 7.95 (d, J=2.6 Hz, 1H), 7.72 (d, J=4.9 Hz, 1H), 7.67 (s, 1H), 7.30 (td, J=8.8, 5.8 Hz, 1H), 7.16-7.05 (m, 1H), 4.51-4.37 (m, 1H), 3.96 (s, 3H), 3.68-3.58 (m, 1H), 3.25 (s, 1H), 2.83 (ddd, J=17.6, 11.5, 5.9 Hz, 1H), 2.71 (d, J=4.7 Hz, 3H), 2.44-2.26 (m, 1H), 1.92 (d, J=13.5 Hz, 1H), 1.00 (d, J=6.6 Hz, 3H).

### Example 112: Synthesis of 5-cyano-N-[2,4-difluoro-3-[5-(1H-imidazol-2-yl)imidazo[1,5-b]pyridazin-2-yl]phenyl]-2-methoxypyridine-3-sulfonamide (Compound 97)

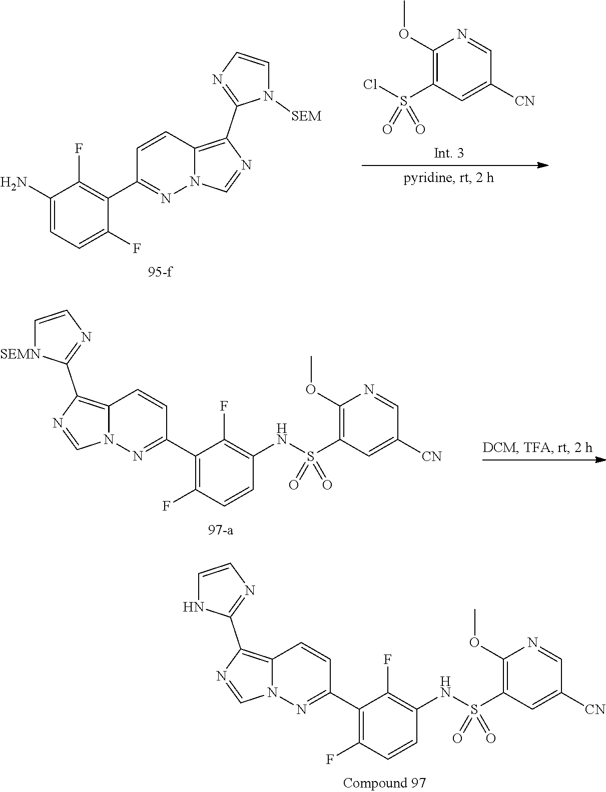 [SMILES: C[Si](C)(C)CCOCn1ccnc1-c1ncn2nc(-c3c(F)ccc(N)c3F)ccc12 ; COc1ncc(C#N)cc1S(=O)(=O)Cl ; COc1ncc(C#N)cc1S(=O)(=O)Nc1ccc(F)c(-c2ccc3c(-c4nccn4COCC[Si](C)(C)C)ncn3n2)c1F ; COc1ncc(C#N)cc1S(=O)(=O)Nc1ccc(F)c(-c2ccc3c(-c4ncc[nH]4)ncn3n2)c1F]

### Synthesis of 97-a: 5-cyano-N-[2,4-difluoro-3-[5-(1-[[2-(trimethylsilyl)ethoxy]methyl]imidazol-2-yl) imidazo[1,5-b]pyridazin-2-yl]phenyl]-2-methoxypyridine-3-sulfonamide

To a stirred solution of 2,4-difluoro-3-[5-(1-[[2-(trimethylsilyl)ethoxy]methyl]imidazol-2-yl)imidazo[1,5-b]pyridazin-2-yl]aniline (120 mg, 0.3 mmol, 1 equiv) in pyridine (2 mL) was added 5-cyano-2-methoxypyridine-3-sulfonyl chloride (94 mg, 0.4 mmol, 1.5 equiv) in portions at room temperature. The resulting mixture was stirred for 2 h then concentrated under reduced pressure. The residue was purified by Flash-Prep-HPLC with the following conditions: Column, WelFlash™ C18-I, Spherical C18 20-40 m, 120 g; mobile phase: 5-70% MeCN/0.1% aqueous formic acid; Detector, 220 nm; to afford 5-cyano-N-[2,4-difluoro-3-[5-(1-[[2-(trimethylsilyl)ethoxy]methyl]imidazol-2-yl)imidazo[1,5-b]pyridazin-2-yl]phenyl]-2-methoxypyridine-3-sulfonamide (105 mg, 61% yield) as a yellow solid.

LCMS (ES, m/z): [M+H]⁺: 639

### Synthesis of Compound 97: 5-cyano-N-[2,4-difluoro-3-[5-(1H-imidazol-2-yl)imidazo[1,5-b]pyridazin-2-yl]phenyl]-2-methoxypyridine-3-sulfonamide

Into a 50 mL round-bottom flask, was placed 5-cyano-N-[2,4-difluoro-3-[5-(1-[[2-(trimethylsilyl)ethoxy]methyl]imidazol-2-yl)imidazo[1,5-b]pyridazin-2-yl]phenyl]-2-methoxypyridine-3-sulfonamide (100 mg, 0.16 mmol, 1 equiv), DCM (6 mL) and TFA (2 mL). The resulting solution was stirred for 2 h, then was concentrated. The residue was dissolved in 3 mL of MeOH and the pH adjusted to 8 with ammonia (7 M in MeOH). After concentration, the residue was purified by prep-HPLC with the following conditions: Column, welch Vltimate XB-C18, 50×250 mm, 10 μm; Mobile Phase: 5-35% MeCN/0.1% aqueous formic acid; Detector, 220 nm; to give 5-cyano-N-[2,4-difluoro-3-[5-(1H-imidazol-2-yl)imidazo[1,5-b]pyridazin-2-yl]phenyl]-2-methoxypyridine-3-sulfonamide (50 mg, 63% yield) as a yellow solid.

LCMS (ES, m/z): [M+H]⁺:509

¹H NMR (300 MHz, DMSO-d₆) δ 8.87 (d, J=2.0 Hz, 2H), 8.64 (d, J=9.4 Hz, 1H), 8.46 (d, J=2.3 Hz, 1H), 7.46 (td, J=9.1, 5.9 Hz, 1H), 7.25-7.15 (m, 1H), 7.12 (s, 2H), 6.94 (dt, J=9.3, 1.2 Hz, 1H), 3.99 (s, 3H).

### Example 113: Synthesis of N-[2,4-difluoro-3-[5-(1H-imidazol-2-yl)imidazo[1,5-b]pyridazin-2-yl]phenyl]-5-fluoro-2-methoxypyridine-3-sulfonamide (Compound 98)

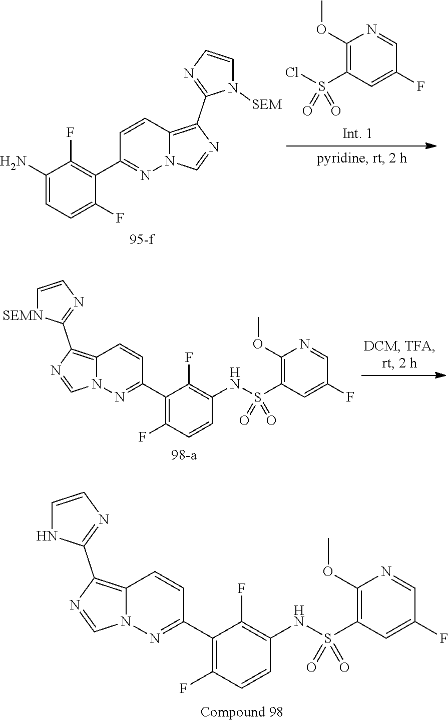 [SMILES: C[Si](C)(C)CCOCn1ccnc1-c1ncn2nc(-c3c(F)ccc(N)c3F)ccc12 ; COc1ncc(F)cc1S(=O)(=O)Cl ; COc1ncc(F)cc1S(=O)(=O)Nc1ccc(F)c(-c2ccc3c(-c4nccn4COCC[Si](C)(C)C)ncn3n2)c1F ; COc1ncc(F)cc1S(=O)(=O)Nc1ccc(F)c(-c2ccc3c(-c4ncc[nH]4)ncn3n2)c1F]

### Synthesis of 98-a: N-[2,4-difluoro-3-[5-(1-[[2-(trimethylsilyl)ethoxy]methyl]imidazol-2-yl)imidazo[1,5-b]pyridazin-2-yl]phenyl]-5-fluoro-2-methoxypyridine-3-sulfonamide

To a stirred solution of 2,4-difluoro-3-[5-(1-[[2-(trimethylsilyl)ethoxy]methyl]imidazol-2-yl)imidazo[1,5-b]pyridazin-2-yl]aniline (120 mg, 0.3 mmol, 1 equiv) in pyridine (2 mL) was added 5-fluoro-2-methoxypyridine-3-sulfonyl chloride (92 mg, 0.4 mmol, 1.5 equiv) in portions at room temperature. The resulting mixture was stirred for 2 h then concentrated under reduced pressure. The residue was purified by Flash-Prep-HPLC with the following conditions: Column, WelFlash™ C18-I, Spherical C18 20-40 m, 120 g; mobile phase: 5-70% MeCN/0.1% aqueous formic acid; Detector, 220 nm; to afford N-[2,4-difluoro-3-[5-(1-[[2-(trimethylsilyl)ethoxy]methyl]imidazol-2-yl)imidazo[1,5-b]pyridazin-2-yl]phenyl]-5-fluoro-2-methoxypyridine-3-sulfonamide (130 mg, 76% yield) as a yellow solid.

LCMS (ES, m/z): [M+H]⁺: 632

### Synthesis of Compound 98: N-[2,4-difluoro-3-[5-(1H-imidazol-2-yl)imidazo[1,5-b]pyridazin-2-yl]phenyl]-5-fluoro-2-methoxypyridine-3-sulfonamide

Into a 50 mL round-bottom flask was placed N-[2,4-difluoro-3-[5-(1-[[2-(trimethylsilyl)ethoxy]methyl]imidazol-2-yl)imidazo[1,5-b]pyridazin-2-yl]phenyl]-5-fluoro-2-methoxypyridine-3-sulfonamide (125 mg, 0.2 mmol, 1 equiv), DCM (6 mL) and TFA (2 mL). The resulting solution was stirred for 2 h then concentrated. The residue was dissolved in 3 mL of MeOH and the pH adjusted to 8 with ammonia in MeOH (7 M) before it was concentrated. The residue was purified by prep-HPLC with the following conditions: Column, welch Vltimate XB-C18, 50×250 mm, 10 μm; Mobile Phase: 5-35% MeCN/0.1% aqueous formic acid; Detector, 220 nm; to give N-[2,4-difluoro-3-[5-(1H-imidazol-2-yl)imidazo[1,5-b]pyridazin-2-yl]phenyl]-5-fluoro-2-methoxypyridine-3-sulfonamide (50 mg, 50% yield) as a yellow solid.

LCMS (ES, m/z): [M+H]⁺:502

¹H NMR (300 MHz, DMSO-d₆) δ 8.88 (s, 1H), 8.72-8.55 (m, 1H), 8.45 (d, J=3.0 Hz, 1H), 8.03 (dd, J=7.4, 3.0 Hz, 1H), 7.48 (td, J=9.0, 5.9 Hz, 1H), 7.26 (t, J=9.0 Hz, 1H), 7.12 (s, 2H), 6.94 (dt, J=9.4, 1.3 Hz, 1H), 3.90 (s, 3H).

### Example 114: Synthesis of 5-chloro-N-(2,4-difluoro-3-(1-(isoxazol-5-yl)imidazo[1,5-a]pyridin-6-yl)phenyl)-2-methoxypyridine-3-sulfonamide (Compound 99))

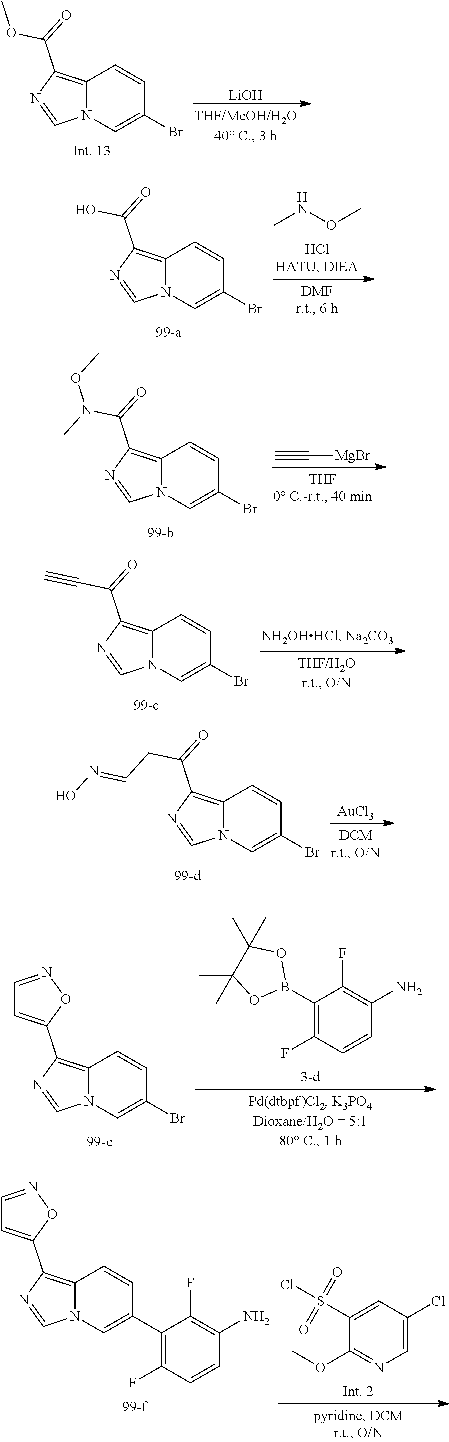

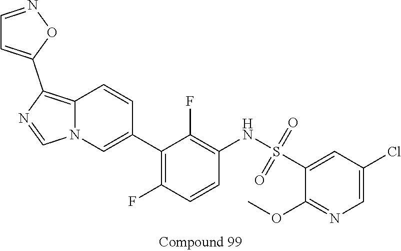 [SMILES: COc1ncc(Cl)cc1S(=O)(=O)Nc1ccc(F)c(-c2ccc3c(-c4ccno4)ncn3c2)c1F]

### Synthesis of 99-a: 6-bromoimidazo[1,5-a]pyridine-1-carboxylic acid

To a stirred solution of methyl 6-bromoimidazo[1,5-a]pyridine-1-carboxylate (5 g, 19.6 mmol, 1 equiv) in THE (33 mL), MeOH (33 mL) and H₂O (33 mL) was added LiOH (2.5 g, 59 mmol, 3 equiv) and the reaction stirred at 40° C. for 3 h. The resulting solution was concentrated under vacuum to remove MeOH and THF, and the aqueous was acidified to pH 3 with 3 M HCl (20 mL). The precipitate was collected by filtration to afford 6-bromoimidazo[1,5-a]pyridine-1-carboxylic acid (4.5 g, 95% yield) as a grey solid.

LCMS (ES, m/z): [M+H]⁺: 241, 243

### Synthesis of 99-b: 6-bromo-N-methoxy-N-methylimidazo[1,5-a]pyridine-1-carboxamide

To a stirred solution of 6-bromoimidazo[1,5-a]pyridine-1-carboxylic acid (3.5 g, 14.5 mmol, 1 equiv) and methoxy(methyl)amine hydrochloride (2.8 g, 29 mmol, 2 equiv) in DMF (70 mL) was added HATU (6.6 g, 17.4 mmol, 1.2 equiv) and DIEA (5.6 g, 43.5 mmol, 3 equiv). The reaction was stirred at room temperature for 6 h, then diluted with H₂O (150 mL). This was extracted with EA (3×50 mL). The combined organics were washed with brine (2×30 mL), dried over anhydrous Na₂SO₄ and concentrated under reduced pressure. The residue was purified by trituration with H₂O (20 mL) and dried to afford pure 6-bromo-N-methoxy-N-methylimidazo[1,5-a]pyridine-1-carboxamide (3.5 g, 85% yield) as a white solid.

LCMS (ES, m/z): [M+H]⁺: 284, 286

### Synthesis of 99-c: 1-(6-bromoimidazo[1,5-a]pyridin-1-yl)prop-2-yn-1-one

To a stirred solution of 6-bromo-N-methoxy-N-methylimidazo[1,5-a]pyridine-1-carboxamide (600 mg, 2.1 mmol, 1 equiv) in THE (20 mL) was added bromo(ethynyl)magnesium (12.6 mL, 6.3 mmol, 3 equiv) dropwise at 0° C. under N₂ atmosphere. The reaction was stirred at room temperature for 40 min, then quenched by addition of H₂O (10 mL). This was extracted with EA (3×10 mL), and the combined organics were washed with brine (5 mL), dried over anhydrous Na₂SO₄ and concentrated under reduced pressure, to give crude 1-[6-bromoimidazo[1,5-a]pyridin-1-yl]prop-2-yn-1-one (500 mg, crude) as a red solid.

LCMS (ES, m/z): [M+H]⁺: 249, 251

### Synthesis of 99-d: 3-(6-bromoimidazo[1,5-a]pyridin-1-yl)-3-oxopropanal oxime

To a stirred solution of 1-[6-bromoimidazo[1,5-a]pyridin-1-yl]prop-2-yn-1-one (500 mg, 2 mmol, 1 equiv) in THE (10 mL) and H₂O (10 mL) was added Na₂CO₃ (638 mg, 6 mmol, 3 equiv) and NH₂OH·HCl (279 mg, 4 mmol, 2 equiv). The reaction was stirred at room temperature overnight. The resulting mixture was extracted with EA (3×10 mL), and the combined organics were washed with brine (10 mL), dried over anhydrous Na₂SO₄ and concentrated under reduced pressure to afford crude 1-[6-bromoimidazo[1,5-a]pyridin-1-yl]-3-(N-hydroxyamino)prop-2-en-1-one (600 mg, crude) as a red solid.

LCMS (ES, m/z): [M+H]⁺: 282, 284

### Synthesis of 99-e: 5-(6-bromoimidazo[1,5-a]pyridin-1-yl)isoxazole

To a stirred solution of 1-[6-bromoimidazo[1,5-a]pyridin-1-yl]-3-(N-hydroxyamino)prop-2-en-1-one (350 mg, 1.2 mmol, 1 equiv) in DCM (10 mL) was added gold (III) trichloride (19 mg, 0.062 mmol, 0.05 equiv). The reaction was stirred at room temperature overnight, then concentrated. The residue was purified by silica gel column chromatography, eluting with PE:EA (1:1) to afford 5-[6-bromoimidazo[1,5-a]pyridin-1-yl]-1,2-oxazole (250 mg, 76% yield) as a yellow solid.

LCMS (ES, m/z): [M+H]⁺: 264, 266

### Synthesis of 99-f: 2,4-difluoro-3-(1-(isoxazol-5-yl)imidazo[1,5-a]pyridin-6-yl)aniline

To a stirred solution of 5-[6-bromoimidazo[1,5-a]pyridin-1-yl]-1,2-oxazole (500 mg, 1.9 mmol, 1 equiv) and 2,4-difluoro-3-(4,4,5,5-tetramethyl-1,3,2-dioxaborolan-2-yl)aniline (724 mg, 2.8 mmol, 1.5 equiv) in dioxane (15 mL) and H₂O (3 mL) were added K₃PO₄ (804 mg, 3.8 mmol, 2 equiv) and Pd(dtbpf)Cl₂ (123 mg, 0.19 mmol, 0.1 equiv). The reaction was stirred at 80° C. for 1 h under a nitrogen atmosphere, then cooled to room temperature and diluted with H₂O (5 mL). The mixture was extracted with EA (3×10 mL), and the combined organics were washed with brine (5 mL), dried over anhydrous Na₂SO₄ and concentrated under reduced pressure. The residue was purified by silica gel column chromatography, eluting with PE:EA (1:1) to afford 2,4-difluoro-3-[1-(1,2-oxazol-5-yl)imidazo[1,5-a]pyridin-6-yl]aniline (400 mg, 67% yield) as a yellow solid.

LCMS (ES, m/z): [M+H]⁺: 313

### Synthesis of Compound 99: 5-chloro-N-(2,4-difluoro-3-(1-(isoxazol-5-yl)imidazo[1,5-a]pyridin-6-yl)phenyl)-2-methoxypyridine-3-sulfonamide

To a stirred solution of 2,4-difluoro-3-[1-(1,2-oxazol-5-yl)imidazo[1,5-a]pyridin-6-yl]aniline (50 mg, 0.16 mmol, 1 equiv) and 5-chloro-2-methoxypyridine-3-sulfonyl chloride (46 mg, 0.19 mmol, 1.2 equiv) in DCM (5 mL) was added pyridine (38 mg, 0.48 mmol, 3 equiv). The reaction was stirred at room temperature overnight, then concentrated. The residue was purified by prep-HPLC with the following conditions: Column, welch Vltimate XB-C18, 50×250 mm, 10 μm; Mobile Phase: 30-65% MeCN/0.1% aqueous formic acid; Detector, 220 nm; to afford 5-chloro-N-(2,4-difluoro-3-(1-(isoxazol-5-yl)imidazo[1,5-a]pyridin-6-yl)phenyl)-2-methoxypyridine-3-sulfonamide (20 mg, 24% yield) as a white solid.

LCMS (ES, m/z): [M+H]⁺: 518

¹H NMR (300 MHz, DMSO-d₆) δ 10.47 (s, 1H), 8.70-8.61 (m, 3H), 8.49 (d, J=2.6 Hz, 1H), 8.14-8.02 (m, 2H), 7.38 (td, J=8.9, 5.8 Hz, 1H), 7.29-7.19 (m, 1H), 7.06 (dd, J=9.5, 1.6 Hz, 1H), 6.78 (d, J=1.9 Hz, 1H), 3.92 (s, 3H).

### Example 115: Synthesis of 5-chloro-N-[2,4-difluoro-3-[1-(1-hydroxyethyl)imidazo[1,5-a]pyridin-6-yl]phenyl]-2-methoxypyridine-3-sulfonamide (Compound 100)

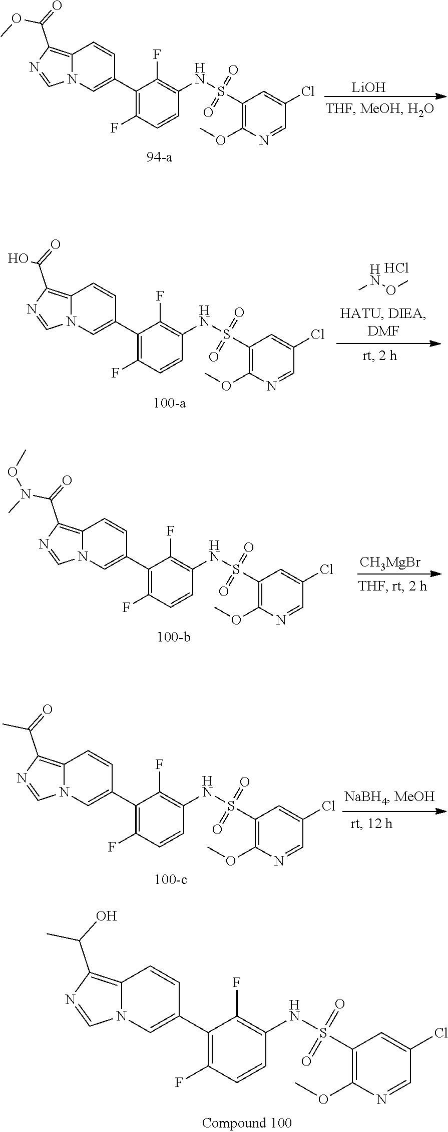

### Synthesis of 100-a: 6-[3-(5-chloro-2-methoxypyridine-3-sulfonamido)-2,6-difluorophenyl]imidazo[1,5-a]pyridine-1-carboxylic acid

To a stirred solution of methyl 6-[3-(5-chloro-2-methoxypyridine-3-sulfonamido)-2,6-difluorophenyl]imidazo[1,5-a]pyridine-1-carboxylate (100 mg, 0.2 mmol, 1 equiv) in THF (2 mL) and MeOH (2 mL) was added a solution of LiOH (24 mg, 1 mmol, 5 equiv) in H₂O (1 mL). The resulting mixture was stirred for 2 h, then diluted with water (10 mL) and acidified to pH 3 with 1 M aqueous HCl. The resulting mixture was extracted with EA (3×10 mL). The combined organic layers were washed with brine (10 mL), dried over anhydrous Na₂SO₄ and concentrated under reduced pressure to give 6-[3-(5-chloro-2-methoxypyridine-3-sulfonamido)-2,6-difluorophenyl]imidazo[1,5-a]pyridine-1-carboxylic acid (80 mg, crude) as a yellow oil which was used in the next step directly without further purification

LCMS (ES, m/z): [M+H]⁺: 495

### Synthesis of 100-b: 6-[3-(5-chloro-2-methoxypyridine-3-sulfonamido)-2,6-difluorophenyl]-N-methoxy-N-methylimidazo[1,5-a]pyridine-1-carboxamide

Into a 50 mL round-bottom flask, was placed 6-[3-(5-chloro-2-methoxypyridine-3-sulfonamido)-2,6-difluorophenyl]imidazo[1,5-a]pyridine-1-carboxylic acid (1 g, 2 mmol, 1 equiv), DMF (15 mL), methoxy(methyl)amine hydrochloride (394 mg, 4 mmol, 2 equiv), HATU (1.15 g, 3 mmol, 1.5 equiv) and DIEA (522 mg, 4 mmol, 2 equiv) at room temperature. The resulting solution was stirred for 2 h at room temperature, then diluted with 60 mL of H₂O and extracted with 3×20 mL of ethyl acetate. The combined organics were washed with 10 mL of brine, dried over anhydrous sodium sulfate, and concentrated under vacuum. The residue was purified by Flash-Prep-HPLC with the following conditions: Column, WelFlash™ C18-I, Spherical C18 20-40 m, 120 g; mobile phase: 30-80% MeCN/0.1% aqueous formic acid to afford 6-[3-(5-chloro-2-methoxypyridine-3-sulfonamido)-2,6-difluorophenyl]-N-methoxy-N-methylimidazo[1,5-a]pyridine-1-carboxamide (730 mg, 67% yield) as a white solid.

LCMS (ES, m/z): [M+H]⁺: 538

### Synthesis of 100-c: N-(3-[1-acetylimidazo[1,5-a]pyridin-6-yl]-2,4-difluorophenyl)-5-chloro-2-methoxypyridine-3-sulfonamide

To a stirred solution of 6-[3-(5-chloro-2-methoxypyridine-3-sulfonamido)-2,6-difluorophenyl]-N-methoxy-N-methylimidazo[1,5-a]pyridine-1-carboxamide (500 mg, 0.9 mmol, 1 equiv) in THE (10 mL) was added methylmagnesium bromide (9 mL, 2.7 mmol, 3 equiv, 3 M in THF) at 0° C. The resulting solution was stirred for 2 h at room temperature, then quenched with saturated aqueous NH₄Cl (20 mL) and extracted with 3×20 mL of ethyl acetate. The combined organics were washed with 10 mL of brine, dried over anhydrous sodium sulfate and concentrated under vacuum. The residue was purified by Flash-Prep-HPLC with the following conditions: Column, WelFlash™ C18-I, Spherical C18 20-40 m, 120 g; mobile phase: 30-80% MeCN/0.1% aqueous formic acid; to afford N-(3-[1-acetylimidazo[1,5-a]pyridin-6-yl]-2,4-difluorophenyl)-5-chloro-2-methoxypyridine-3-sulfonamide (300 mg, 65% yield) as a yellow solid.

LCMS (ES, m/z): [M+H]⁺: 493

### Synthesis of Compound 100: 5-chloro-N-[2,4-difluoro-3-[1-(1-hydroxyethyl)imidazo[1,5-a]pyridin-6-yl]phenyl]-2-methoxypyridine-3-sulfonamide

To a stirred solution of N-(3-[1-acetylimidazo[1,5-a]pyridin-6-yl]-2,4-difluorophenyl)-5-chloro-2-methoxypyridine-3-sulfonamide (80 mg, 0.2 mmol, 1 equiv) in MeOH (4 mL) was added NaBH₄ (37 mg, 1 mmol, 6 equiv) in portions at room temperature.

The resulting solution was stirred for 6 h then quenched by the addition of 5 mL of saturated aqueous NH₄C1, and extracted with 4×5 mL of ethyl acetate. The combined organics were washed with 10 mL of brine, dried over anhydrous sodium sulfate and concentrated under vacuum. The residue was purified by prep-HPLC with the following conditions: Column, welch Vltimate XB-C18, 50×250 mm, 10 μm; Mobile Phase: 15-45% MeCN/0.1% aqueous formic acid; to afford 5-chloro-N-[2,4-difluoro-3-[1-(1-hydroxyethyl)imidazo[1,5-a]pyridin-6-yl]phenyl]-2-methoxypyridine-3-sulfonamide (9.4 mg, 12% yield) as a white solid.

LCMS (ES, m/z): [M+H]⁺: 495

¹H NMR (300 MHz, DMSO-d₆) δ 10.42 (s, 1H), 8.50 (d, J=2.6 Hz, 1H), 8.35 (d, J=15.8 Hz, 2H), 8.07 (d, J=2.6 Hz, 1H), 7.76 (d, J=9.5 Hz, 1H), 7.34 (td, J=8.9, 5.8 Hz, 1H), 7.21 (td, J=9.1, 1.6 Hz, 1H), 6.62-6.52 (m, 1H), 5.14-5.01 (m, 2H), 3.92 (s, 3H), 1.49 (d, J=6.1 Hz, 3H).

### Example 116: Synthesis of 6-(2-chloro-3-((5-chloro-2-methoxypyridine)-3-sulfonamido)-6-fluorophenyl)-N-methylimidazo[1,5-a]pyridine-1-carboxamide (Compound 101)

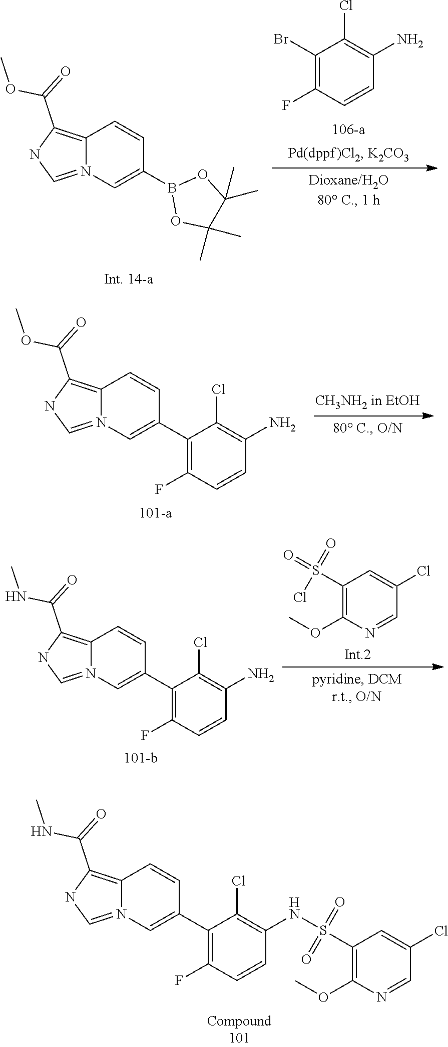

### Synthesis of 101-a: methyl 6-(3-amino-2-chloro-6-fluorophenyl)imidazo[1,5-a]pyridine-1-carboxylate

To a solution of 3-bromo-2-chloro-4-fluoroaniline (0.4 g, 1.8 mmol, 1 equiv) and methyl 6-(4,4,5,5-tetramethyl-1,3,2-dioxaborolan-2-yl)imidazo[1,5-a]pyridine-1-carboxylate (1 g, 3.2 mmol, 1.8 equiv) in dioxane (16 mL) and H₂O (4 mL) were added K₂CO₃ (0.5 g, 3.6 mmol, 2 equiv) and Pd(dppf)Cl₂ (0.13 g, 0.18 mmol, 0.1 equiv). The reaction was stirred at 80° C. for 1 h under a nitrogen atmosphere, then the resulting mixture was cooled, diluted with H₂O (10 mL) and extracted with EA (3×10 mL). The combined organics were washed with brine (10 mL), dried over anhydrous Na₂SO₄ and concentrated under reduced pressure. The residue was purified by silica gel column chromatography, eluting with PE:EA (1:1) to afford methyl 6-(3-amino-2-chloro-6-fluorophenyl)imidazo[1,5-a]pyridine-1-carboxylate (450 mg, 79% yield) as a grey solid.

LCMS (ES, m/z): [M+H]⁺: 320

### Synthesis of 101-b: 6-(3-amino-2-chloro-6-fluorophenyl)-N-methylimidazo[1,5-a]pyridine-1-carboxamide

Methyl 6-(3-amino-2-chloro-6-fluorophenyl)imidazo[1,5-a]pyridine-1-carboxylate (400 mg, 1.2 mmol, 1 equiv) was dissolved in methylamine solution (30% in ethanol, 20 mL) and stirred at 80° C. overnight. The resulting solution was concentrated directly to afford crude 6-(3-amino-2-chloro-6-fluorophenyl)-N-methylimidazo[1,5-a]pyridine-1-carboxamide (380 mg, crude) as a red solid which was used for next step directly without further purification.

LCMS (ES, m/z): [M+H]⁺: 319

### Synthesis of Compound 101: 6-(2-chloro-3-((5-chloro-2-methoxypyridine)-3-sulfonamido)-6-fluorophenyl)-N-methylimidazo[1,5-a]pyridine-1-carboxamide

To a stirred solution of 6-(3-amino-2-chloro-6-fluorophenyl)-N-methylimidazo[1,5-a]pyridine-1-carboxamide (100 mg, 0.3 mmol, 1 equiv) in DCM (5 mL) was added 5-chloro-2-methoxypyridine-3-sulfonyl chloride (91 mg, 0.37 mmol, 1.2 equiv) and pyridine (74 mg, 0.9 mmol, 3 equiv). The reaction was stirred at room temperature overnight. The resulting solution was concentrated and the residue was purified by prep-HPLC with the following conditions: Column, welch Vltimate XB-C18, 50×250 mm, 10 μm; Mobile Phase: 20-60% MeCN/0.1% aqueous formic acid; Detector, 220 nm; to afford 6-[2-chloro-3-(5-chloro-2-methoxypyridine-3-sulfonamido)-6-fluorophenyl]-N-methylimidazo[1,5-a]pyridine-1-carboxamide (100 mg, 61% yield) as a white solid.

LCMS (ES, m/z): [M+H]⁺: 524

¹H NMR (300 MHz, DMSO-d₆) δ 10.42 (s, 1H), 8.55 (d, J=1.3 Hz, 1H), 8.50 (d, J=2.6 Hz, 1H), 8.46 (s, 1H), 8.14 (d, J=9.4 Hz, 1H), 8.10 (d, J=4.9 Hz, 1H), 8.07 (d, J=2.6 Hz, 1H), 7.52-7.35 (m, 2H), 6.96 (dt, J=9.5, 1.0 Hz, 1H), 3.88 (s, 3H), 2.81 (d, J=4.7 Hz, 3H).

### Example 117: Synthesis of 5-chloro-N-(2,4-difluoro-3-[1-[5-(trifluoromethyl)-4H-1,2,4-triazol-3-yl]imidazo[1,5-a]pyridin-6-yl]phenyl)-2-methoxypyridine-3-sulfonamide (Compound 102)

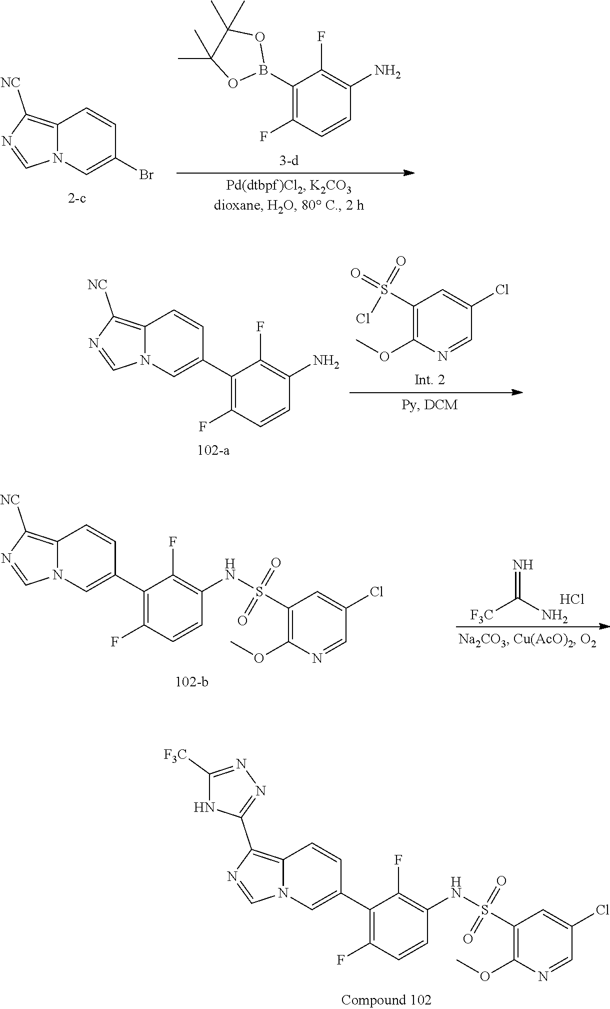

### Synthesis of 102-a: 6-(3-amino-2,6-difluorophenyl)imidazo[1,5-a]pyridine-1-carbonitrile

Into a 40 mL vial were added 6-bromoimidazo[1,5-a]pyridine-1-carbonitrile (600 mg, 2.7 mmol, 1 equiv), 2,4-difluoro-3-(4,4,5,5-tetramethyl-1,3,2-dioxaborolan-2-yl)aniline (1.3 g, 5 mmol, 1.9 equiv), Pd(dtbpf)Cl₂ (158 mg, 0.24 mmol, 0.1 equiv), K₂CO₃ (1.1 g, 8 mmol, 3 equiv), dioxane (12 mL) and H₂O (2.4 mL). The resulting mixture was stirred for 2 h at 80° C. under nitrogen atmosphere, then was cooled and diluted with water (100 mL). The resulting mixture was extracted with EtOAc (3×100 mL), and the combined organics were washed with brine (3×50 mL), dried over anhydrous Na₂SO₄ and concentrated under reduced pressure. The residue was purified by silica gel column chromatography, eluting with PE/EA (1:1) to afford 6-(3-amino-2,6-difluorophenyl)imidazo[1,5-a]pyridine-1-carbonitrile (570 mg, 78% yield) as a light brown solid.

LCMS (ES, m/z): [M+H]⁺: 271

### Synthesis of 102-b: N-(3-[1-cyanoimidazo[1,5-a]pyridin-6-yl]-2,4-difluorophenyl)-5-fluoro-2-methoxypyridine-3-sulfonamide

Into a 40 mL vial were added 6-(3-amino-2,6-difluorophenyl)imidazo[1,5-a]pyridine-1-carbonitrile (250 mg, 0.9 mmol, 1 equiv), 5-fluoro-2-methoxypyridine-3-sulfonyl chloride (310 mg, 1.4 mmol, 1.5 equiv), DCM (5 mL) and pyridine (365 mg, 4.6 mmol, 5 equiv). The resulting mixture was stirred overnight at room temperature, then concentrated under vacuum. The residue was purified by silica gel column chromatography, eluting with PE/THF (2:1) to afford N-(3-[1-cyanoimidazo[1,5-a]pyridin-6-yl]-2,4-difluorophenyl)-5-fluoro-2-methoxypyridine-3-sulfonamide (290 mg, 68% yield) as a brown solid.

LCMS (ES, m/z): [M+H]⁺: 476

### Synthesis of Compound 102: 5-chloro-N-(2,4-difluoro-3-[1-[5-(trifluoromethyl)-4H-1,2,4-triazol-3-yl]imidazo[1,5-a]pyridin-6-yl]phenyl)-2-methoxypyridine-3-sulfonamide

Into a 50 mL round-bottom flask were added 5-chloro-N-(3-[1-cyanoimidazo[1,5-a]pyridin-6-yl]-2,4-difluorophenyl)-2-methoxypyridine-3-sulfonamide (200 mg, 0.42 mmol, 1 equiv), trifluoroethanimidamide hydrochloride (312 mg, 2.1 mmol, 5 equiv), Cu(AcO)₂ (8 mg, 0.04 mmol, 0.1 equiv), Na₂CO₃ (133 mg, 1.3 mmol, 3 equiv) and DMSO (4 mL). The resulting mixture was stirred for 4 h at 120° C. under O₂ atmosphere. The mixture was allowed to cool and was diluted with water (100 mL). The resulting mixture was extracted with EtOAc (3×100 mL), and the combined organics were washed with brine (3×50 mL), dried over anhydrous Na₂SO₄ and concentrated under reduced pressure. The residue was purified by silica gel column chromatography, eluting with PE/THF (1:1) to afford 5-chloro-N-(2,4-difluoro-3-[1-[5-(trifluoromethyl)-4H-1,2,4-triazol-3-yl]imidazo[1,5-a]pyridin-6-yl]phenyl)-2-methoxypyridine-3-sulfonamide (28 mg, 11% yield) as a white solid.

LCMS (ES, m/z): [M+H]⁺: 586

¹H NMR (300 MHz, DMSO-d₆) δ 15.21 (s, 1H), 10.48 (s, 1H), 8.70 (s, 2H), 8.52 (d, J=2.6 Hz, 1H), 8.19 (d, J=9.4 Hz, 1H), 8.10 (d, J=2.6 Hz, 1H), 7.40 (td, J=8.8, 5.8 Hz, 1H), 7.33-7.22 (m, 1H), 7.16-7.07 (m, 1H), 3.93 (s, 3H).

### Example 118: Synthesis of 5-chloro-N-[2-chloro-4-fluoro-3-[1-(1H-imidazol-2-yl)imidazo[1,5-a]pyridin-6-yl]phenyl]-2-methoxypyridine-3-sulfonamide (Compound 103)

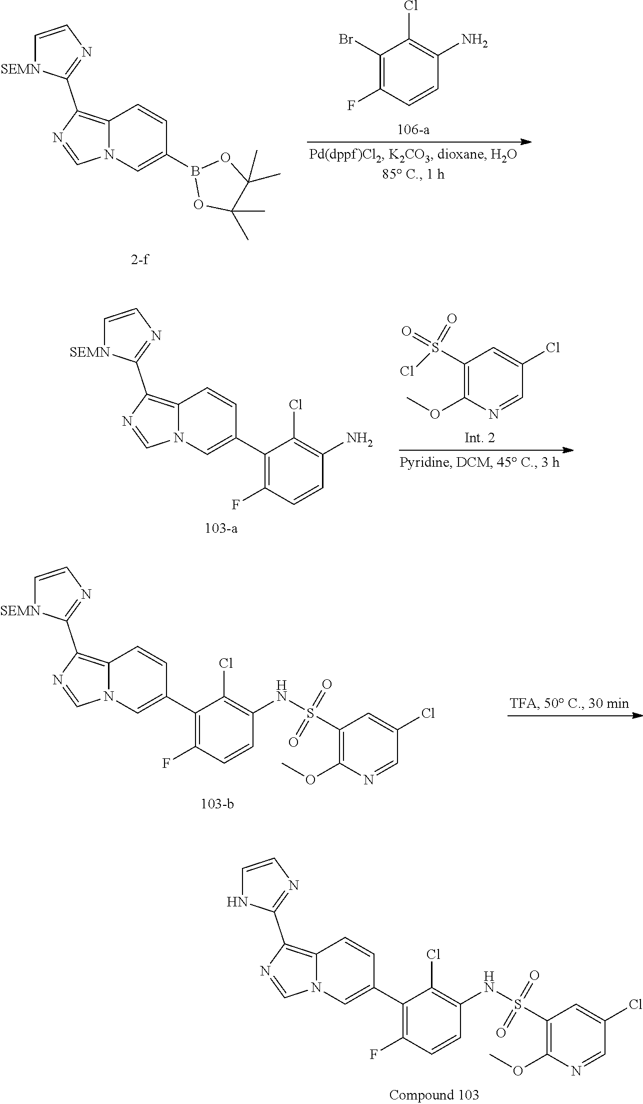 [SMILES: CC1(C)OB(c2ccc3c(-c4nccn4COCC[Si](C)(C)C)ncn3c2)OC1(C)C ; Nc1ccc(F)c(Br)c1Cl ; C[Si](C)(C)CCOCn1ccnc1-c1ncn2cc(-c3c(F)ccc(N)c3Cl)ccc12 ; COc1ncc(Cl)cc1S(=O)(=O)Cl ; COc1ncc(Cl)cc1S(=O)(=O)Nc1ccc(F)c(-c2ccc3c(-c4nccn4COCC[Si](C)(C)C)ncn3c2)c1Cl ; COc1ncc(Cl)cc1S(=O)(=O)Nc1ccc(F)c(-c2ccc3c(-c4ncc[nH]4)ncn3c2)c1Cl]

### Synthesis of 103-a: 2-chloro-4-fluoro-3-[1-(1-[[2-(trimethylsilyl)ethoxy]methyl]imidazol-2-yl)imidazo[1,5-a]pyridin-6-yl]aniline

Into a 25 mL 3-necked round-bottom flask purged and maintained with an inert atmosphere of nitrogen, was placed 2-[6-(4,4,5,5-tetramethyl-1,3,2-dioxaborolan-2-yl)imidazo[1,5-a]pyridin-1-yl]-1-[[2-(trimethylsilyl)ethoxy]methyl]imidazole (200 mg, 0.45 mmol, 1 equiv), 3-bromo-2-chloro-4-fluoroaniline (102 mg, 0.45 mmol, 1 equiv), Pd(dppf)Cl₂ (33 mg, 0.045 mmol, 0.1 equiv), K₂CO₃ (188 mg, 1.36 mmol, 3 equiv), dioxane (10 mL) and H₂O (3 mL). The resulting solution was stirred for 2 h at 85° C., then concentrated under vacuum. The residue was applied to a silica gel column eluting with THF:PE (1:1) to afford 2-chloro-4-fluoro-3-[1-(1-[[2-(trimethylsilyl)ethoxy]methyl]imidazol-2-yl)imidazo[1,5-a]pyridin-6-yl]aniline (131 mg, 50% yield) as a yellow oil.

LCMS (ES, m/z): [M+H]⁺: 458

### Synthesis of 103-b: 5-chloro-N-[2-chloro-4-fluoro-3-[1-(1-[[2-(trimethylsilyl)ethoxy]methyl]imidazol-2-yl)imidazo[1,5-a]pyridin-6-yl]phenyl]-2-methoxypyridine-3-sulfonamide

Into a 25 mL 3-necked round-bottom flask, purged and maintained with an inert atmosphere of nitrogen, was placed 2-chloro-4-fluoro-3-[1-(1-[[2-(trimethylsilyl)ethoxy]methyl]imidazol-2-yl)imidazo[1,5-a]pyridin-6-yl]aniline (131 mg, 0.29 mmol, 1 equiv), 5-chloro-2-methoxypyridine-3-sulfonyl chloride (69 mg, 0.29 mmol, 1 equiv) and pyridine (68 mg, 0.86 mmol, 3 equiv) in DCM (10 mL). The resulting solution was stirred for 2 h at 45° C., then was concentrated under vacuum. The residue was applied onto a silica gel column, eluting with THF:PE (1:1) to give 5-chloro-N-[2-chloro-4-fluoro-3-[1-(1-[[2-(trimethylsilyl)ethoxy]methyl]imidazol-2-yl)imidazo[1,5-a]pyridin-6-yl]phenyl]-2-methoxypyridine-3-sulfonamide (200 mg, 74% yield) as a yellow oil.

LCMS (ES, m/z): [M+H]⁺: 663

### Synthesis of Compound 103: 5-chloro-N-[2-chloro-4-fluoro-3-[1-(1H-imidazol-2-yl)imidazo[1,5-a]pyridin-6-yl]phenyl]-2-methoxypyridine-3-sulfonamide

Into a 25 mL 3-necked round-bottom flask was placed 5-chloro-N-[2-chloro-4-fluoro-3-[1-(1-[[2-(trimethylsilyl)ethoxy]methyl]imidazol-2-yl)imidazo[1,5-a]pyridin-6-yl]phenyl]-2-methoxypyridine-3-sulfonamide (200 mg, 0.3 mmol, 1 equiv) and TFA (3 mL). The resulting solution was stirred for 30 min at 50° C. then concentrated under vacuum. The crude product was purified by Flash-Prep-HPLC with the following conditions: welch Vltimate XB-C18, 50×250 mm, 10 μm; Mobile Phase: 35-60% MeCN/0.05% aqueous ammonia; detector, 220 nm; to give 5-chloro-N-[2-chloro-4-fluoro-3-[1-(1H-imidazol-2-yl)imidazo[1,5-a]pyridin-6-yl]phenyl]-2-methoxypyridine-3-sulfonamide (30 mg, 19% yield) as a yellow solid.

LCMS (ES, m/z): [M+H]⁺: 533

¹H NMR (300 MHz, DMSO-d₆) δ 8.47 (dd, J=6.3, 3.7 Hz, 3H), 8.22 (d, J=9.4 Hz, 1H), 8.06 (d, J=2.6 Hz, 1H), 7.50-7.30 (m, 2H), 7.07 (s, 2H), 6.79 (d, J=9.4 Hz, 1H), 3.88 (s, 3H).

### Example 119: Synthesis of 5-chloro-N-[2,4-difluoro-3-[5-fluoro-1-(1H-imidazol-2-yl)imidazo[1,5-a]pyridin-6-yl]phenyl]-2-methoxypyridine-3-sulfonamide (Compound 104)

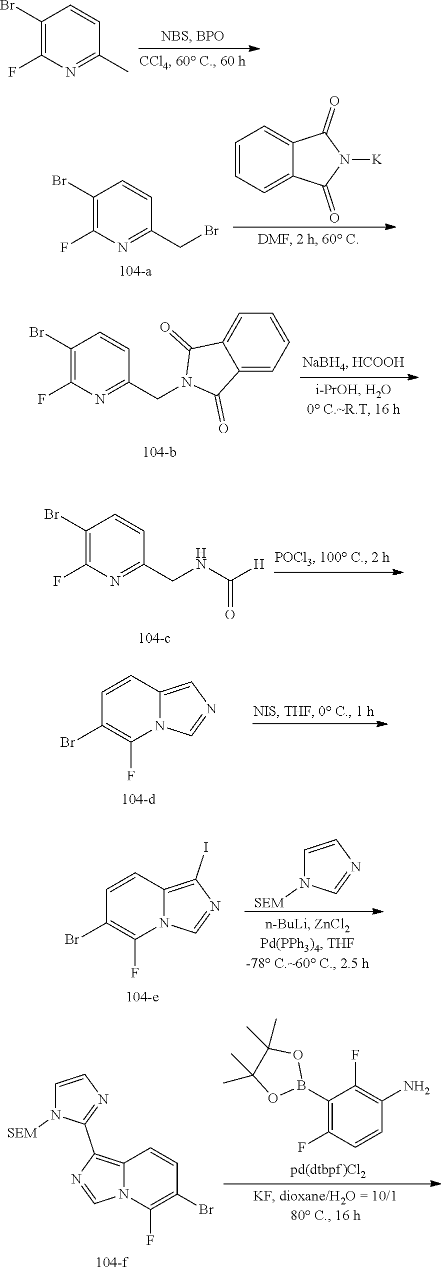

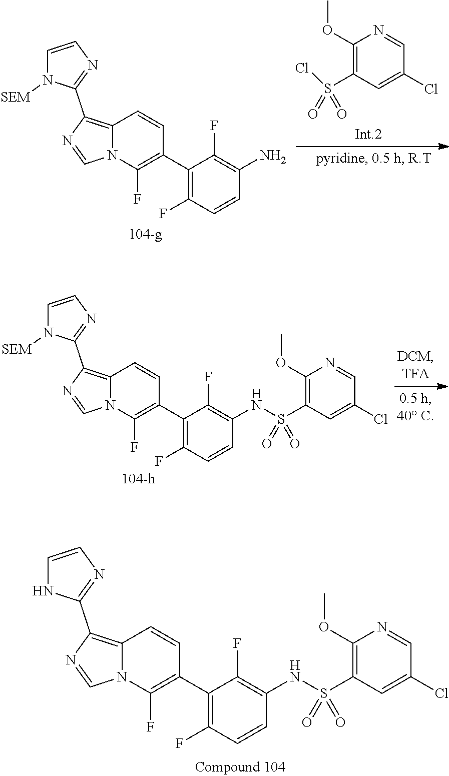 [SMILES: C[Si](C)(C)CCOCn1ccnc1-c1ncn2c(F)c(-c3c(F)ccc(N)c3F)ccc12 ; COc1ncc(Cl)cc1S(=O)(=O)Cl ; COc1ncc(Cl)cc1S(=O)(=O)Nc1ccc(F)c(-c2ccc3c(-c4nccn4COCC[Si](C)(C)C)ncn3c2F)c1F ; COc1ncc(Cl)cc1S(=O)(=O)Nc1ccc(F)c(-c2ccc3c(-c4ncc[nH]4)ncn3c2F)c1F]

### Synthesis of 104-a: 3-bromo-6-(bromomethyl)-2-fluoropyridine

Into a 250 mL round-bottom flask were added 3-bromo-2-fluoro-6-methylpyridine (5 g, 26 mmol, 1 equiv) and CCl₄ (100 mL) at room temperature. Benzoyl peroxide (0.67 g, 2.631 mmol, 0.1 equiv) and NBS (5.15 g, 29 mmol, 1.1 equiv) were added and the resulting mixture was stirred for 60 h at 60° C. under nitrogen atmosphere. The mixture was cooled, filtered and the filtrate was purified by silica gel column chromatography, eluting with PE/EtOAc (2:1) to afford 3-bromo-6-(bromomethyl)-2-fluoropyridine (7.1 g, crude) as a light orange oil.

LCMS (ES, m/z): [M+H]⁺: 268, 270, 272

### Synthesis of 104-b: 2-[(5-bromo-6-fluoropyridin-2-yl)methyl]isoindole-1,3-dione

Into a 250 mL round-bottom flask were added 3-bromo-6-(bromomethyl)-2-fluoropyridine (13 g, 29 mmol, 1 equiv) and DMF (150 mL) at room temperature. Potassium phthalimide (8.29 g, 45 mmol, 1.5 equiv) was added and the resulting mixture was stirred for 2 h at 60° C. The reaction was cooled and diluted with water (1000 mL), before being extracted with EA (3×300 mL). The combined organics were washed with water and brine, dried over anhydrous Na₂SO₄ and concentrated under reduced pressure. The residue was purified by silica gel column chromatography, eluting with PE/EA (2/1) to afford 2-[(5-bromo-6-fluoropyridin-2-yl)methyl]isoindole-1,3-dione (7.5 g, 75% yield) as a yellow solid.

LCMS (ES, m/z): [M+H]⁺: 335, 337.

### Synthesis of 104-c: N-[(5-bromo-6-fluoropyridin-2-yl)methyl]formamide

To a stirred solution of 2-[(5-bromo-6-fluoropyridin-2-yl)methyl]isoindole-1,3-dione (7.5 g, 22 mmol, 1 equiv) in i-PrOH (270 mL) and H₂O (45 mL) were added NaBH₄ (4.2 g, 112 mmol, 5 equiv) in portions at 0° C. The resulting mixture was stirred for 16 h, then HCOOH (63 mL) was added dropwise at room temperature. The resulting mixture was stirred for 24 h at 80° C. After cooling, the reaction was filtered and the filtrate was concentrated under reduced pressure to give N-[(5-bromo-6-fluoropyridin-2-yl)methyl]formamide (12.6 g, crude) as a light brown oil.

LCMS (ES, m/z): [M+H]⁺: 233, 235.

### Synthesis of 104-d: 6-bromo-5-fluoroimidazo[1,5-a]pyridine

To a solution of N-[(5-bromo-6-fluoropyridin-2-yl)methyl]formamide (6 g, 14 mmol, 1 equiv) in toluene (240 mL) was added POCl₃ (10.7 g, 70 mmol, 5 equiv) and the reaction stirred for 2 h at 100° C. The mixture was cooled and concentrated under reduced pressure. The residue was diluted with EA (100 mL) and washed with 30 mL of aqueous NaHCO₃ solution. The organics were washed with water and brine (1×50 mL), dried over anhydrous Na₂SO₄ and concentrated under reduced pressure. The residue was purified by silica gel column chromatography, eluting with PE/EA (1/1) to afford 6-bromo-5-fluoroimidazo[1,5-a]pyridine (1.1 g, 37% yield) as a light brown solid.

LCMS (ES, m/z): [M+H]⁺: 215, 217.

### Synthesis of 104-e: 6-bromo-5-fluoro-1-iodoimidazo[1,5-a]pyridine

Into a 100 mL 3-necked round-bottom flask, was placed 6-bromo-5-fluoroimidazo[1,5-a]pyridine (2.1 g, 9.8 mmol, 1 equiv) in DMF (20 mL). NIS (2.2 g, 9.766 mmol, 1 equiv) was added at 0° C., and the solution was stirred for 60 min at 0° C. in an ice/salt bath. The reaction was quenched with 200 mL of aqueous Na₂S₂O₃ solution, and the solids were collected by filtration. Drying gave 6-bromo-5-fluoro-1-iodoimidazo[1,5-a]pyridine (2.4 g, 72% yield) as a brown solid.

LCMS (ES, m/z): [M+H]⁺: 341, 343.

### Synthesis of 104-f: 2-[6-bromo-5-fluoroimidazo[1,5-a]pyridin-1-yl]-1-[[2-(trimethylsilyl)ethoxy]methyl]imidazole

To a stirred solution of 1-[[2-(trimethylsilyl)ethoxy]methyl]imidazole (1.4 g, 7 mmol, 3 equiv) in THE (10 mL) was added n-BuLi (2.8 mL, 7 mmol, 3 equiv, 2.5 M in hexane) dropwise at −78° C. under N₂ atmosphere. The resulting mixture was stirred for 30 min then ZnCl₂ (960 mg, 7.04 mmol, 3 equiv) was added at −78° C. The resulting mixture was stirred for 30 min at room temperature, before 6-bromo-5-fluoro-1-iodoimidazo[1,5-a]pyridine (800 mg, 2.347 mmol, 1 equiv) and Pd(PPh₃)₄ (542 mg, 0.469 mmol, 0.2 equiv) in THE (5 mL) were added dropwise at room temperature. The resulting mixture was stirred for 30 min at 60° C., then was cooled and quenched by the addition of water (30 mL). This was extracted with EA (3×30 mL), and the combined organics were washed with water (50 mL) and brine (50 mL), dried over anhydrous Na₂SO₄ and concentrated under reduced pressure. The residue was purified by silica gel column chromatography, eluting with PE/EA (3/1) to afford 2-[6-bromo-5-fluoroimidazo[1,5-a]pyridin-1-yl]-1-[[2-(trimethylsilyl)ethoxy]methyl]imidazole (843 mg, 87% yield) as a light brown solid.

LCMS (ES, m/z): [M+H]⁺: 411, 413

### Synthesis of 104-g: 2,4-difluoro-3-[5-fluoro-1-(1-[[2-(trimethylsilyl)ethoxy]methyl]imidazol-2-yl) imidazo[1,5-a]pyridin-6-yl]aniline

To a solution of 2-[6-bromo-5-fluoroimidazo[1,5-a]pyridin-1-yl]-1-[[2-(trimethylsilyl)ethoxy]methyl]imidazole (800 mg, 1.9 mmol, 1 equiv) and 2,4-difluoro-3-(4,4,5,5-tetramethyl-1,3,2-dioxaborolan-2-yl)aniline (992 mg, 3.9 mmol, 2 equiv) in dioxane (2 mL) and H₂O (0.2 mL) were added KF (3.4 g, 5.8 mmol, 3 equiv) and Pd(dtbpf)Cl₂ (254 mg, 0.39 mmol, 0.2 equiv). After stirring for 16 h at 80° C. under a nitrogen atmosphere, the resulting mixture was concentrated under reduced pressure. The residue was purified by silica gel column chromatography, eluting with PE/EA (50-100%) to afford 2,4-difluoro-3-[5-fluoro-1-(1-[[2-(trimethylsilyl)ethoxy]methyl]imidazol-2-yl)imidazo[1,5-a]pyridin-6-yl]aniline (460 mg, 51% yield) as a light brown solid.

LCMS (ES, m/z): [M+H]⁺: 460

### Synthesis of 104-h: 5-chloro-N-[2,4-difluoro-3-[5-fluoro-1-(1-[[2-(trimethylsilyl)ethoxy]methyl]imidazol-2-yl)imidazo[1,5-a]pyridin-6-yl]phenyl]-2-methoxypyridine-3-sulfonamide

To a stirred mixture of 2,4-difluoro-3-[5-fluoro-1-(1-[[2-(trimethylsilyl)ethoxy]methyl]imidazol-2-yl)imidazo[1,5-a]pyridin-6-yl]aniline (100 mg, 0.218 mmol, 1 equiv) in pyridine (1 mL) was added 5-chloro-2-methoxypyridine-3-sulfonyl chloride (105 mg, 0.435 mmol, 2 equiv) at room temperature. The resulting mixture was stirred for 30 min then diluted with water (20 mL). This was extracted with EA (3×20 mL), and the combined organics were washed with water (50 mL) and brine (50 mL), dried over anhydrous Na₂SO₄, then concentrated under reduced pressure. The residue was purified by silica gel column chromatography, eluting with PE/EA (1/1) to afford 5-chloro-N-[2,4-difluoro-3-[5-fluoro-1-(1-[[2-(trimethylsilyl)ethoxy]methyl]imidazol-2-yl)imidazo[1,5-a]pyridin-6-yl]phenyl]-2-methoxypyridine-3-sulfonamide (100 mg, 69% yield) as a light yellow solid.

LCMS (ES, m/z): [M+H]⁺: 665.

### Synthesis of Compound 104: 5-chloro-N-[2,4-difluoro-3-[5-fluoro-1-(1H-imidazol-2-yl)imidazo[1,5-a]pyridin-6-yl phenyl]-2-methoxypyridine-3-sulfonamide

A solution of 5-chloro-N-[2,4-difluoro-3-[5-fluoro-1-(1-[[2-(trimethylsilyl)ethoxy]methyl]imidazol-2-yl)imidazo[1,5-a]pyridin-6-yl]phenyl]-2-methoxypyridine-3-sulfonamide (90 mg, 0.135 mmol, 1 equiv) in DCM (1 mL) and TFA (0.5 mL) was stirred for 30 min at 40° C. The mixture was cooled and concentrated under vacuum. The crude product was purified by Prep-HPLC with the following conditions: Column: Sunfire Prep C18 OBD, 50*250 mm 5 μm 10 nm; Mobile Phase: 5-45% 1:1 MeOH:ACN/0.05% aqueous ammonia; Flow rate: 90 mL/min; to afford 5-chloro-N-[2,4-difluoro-3-[5-fluoro-1-(1H-imidazol-2-yl)imidazo[1,5-a]pyridin-6-yl]phenyl]-2-methoxypyridine-3-sulfonamide (21 mg, 29% yield) as a light yellow solid.

LCMS (ES, m/z): [M+H]⁺: 535.

¹H NMR (300 MHz, DMSO-d6): δ 12.53 (br, 1H), 10.52 (br, 1H), 8.69 (s, 1H), 8.50 (d, J=2.6 Hz, 1H), 8.17-8.04 (m, 2H), 7.53-7.39 (m, 1H), 7.27 (t, J=9.1 Hz, 1H), 7.11 (s, 2H), 7.01-6.89 (m, 1H), 3.91 (s, 3H).

### Example 120: Synthesis of 5-chloro-N-[2,4-difluoro-3-[5-fluoro-1-(2H-pyrazol-3-yl)imidazo[1,5-a]pyridin-6-yl]phenyl]-2-methoxypyridine-3-sulfonamide (Compound 105)

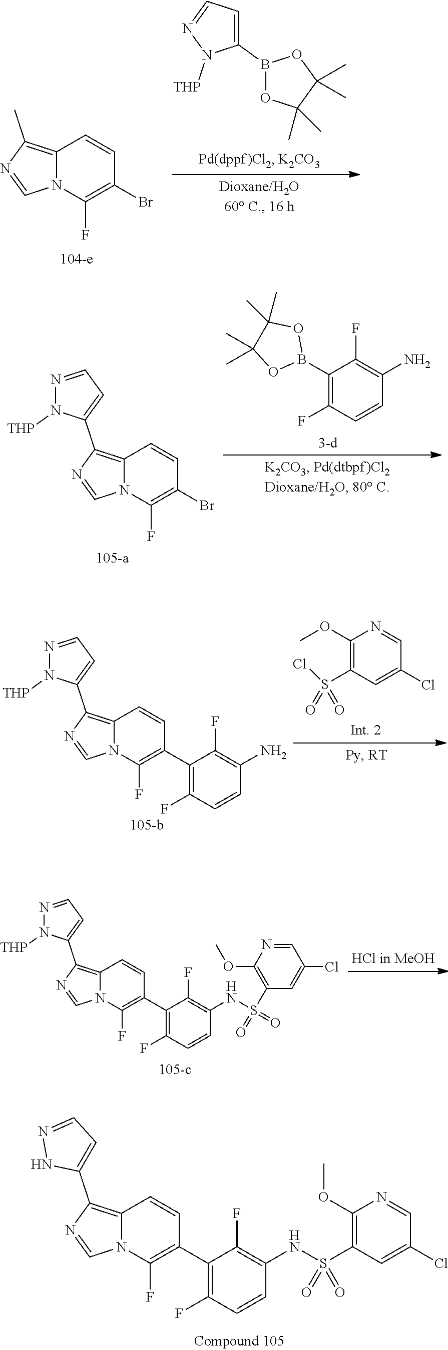

### Synthesis of 105-a: 5-[6-bromo-5-fluoroimidazo[1,5-a]pyridin-1-yl]-1-(oxan-2-yl)pyrazole

A solution of 6-bromo-5-fluoro-1-iodoimidazo[1,5-a]pyridine (200 mg, 0.6 mmol, 1 equiv), 1-(oxan-2-yl)-5-(4,4,5,5-tetramethyl-1,3,2-dioxaborolan-2-yl)pyrazole (163 mg, 0.6 mmol, 1 equiv), K₂CO₃ (162 mg, 1.2 mmol, 2 equiv) and Pd(dppf)Cl₂ (43 mg, 0.06 mmol, 0.1 equiv) in dioxane (2 mL) and H₂O (0.4 mL) was stirred overnight at 60° C. under nitrogen atmosphere. The mixture was cooled and diluted with water (20 mL). The resulting mixture was extracted with EtOAc (3×50 mL), and the combined organics were washed with water and brine, dried over anhydrous Na₂SO₄ and concentrated under reduced pressure. The residue was purified by silica gel column chromatography, eluting with EtOAc/PE (0-30%) to afford 5-[6-bromo-5-fluoroimidazo[1,5-a]pyridin-1-yl]-1-(oxan-2-yl)pyrazole (170 mg, 79% yield) as a yellow oil.

LCMS (ES, m/z): [M+H]⁺: 365, 367.

### Synthesis of 105-b: 2,4-difluoro-3-[5-fluoro-1-[2-(oxan-2-yl)pyrazol-3-yl]imidazo[1,5-a]pyridin-6-yl]aniline

A solution of 5-[6-bromo-5-fluoroimidazo[1,5-a]pyridin-1-yl]-1-(oxan-2-yl)pyrazole (150 mg, 0.41 mmol, 1 equiv), 2,4-difluoro-3-(4,4,5-trimethyl-1,3,2-dioxaborolan-2-yl)aniline (198 mg, 0.82 mmol, 2 equiv), KF (72 mg, 1.24 mmol, 3 equiv) and Pd(dtbpf)Cl₂ (81 mg, 0.12 mmol, 0.3 equiv) in dioxane (1.5 mL) and H₂O (0.3 mL) was stirred for 6 h at 80° C. under nitrogen atmosphere. The mixture was cooled and diluted with water (5 mL). The resulting mixture was extracted with EtOAc (3×20 mL), and the combined organics were washed with water and brine, dried over anhydrous Na₂SO₄ and concentrated under reduced pressure. The residue was purified by silica gel column chromatography, eluting with EtOAc/PE (0-50%) to afford 2,4-difluoro-3-[5-fluoro-1-[2-(oxan-2-yl)pyrazol-3-yl]imidazo[1,5-a]pyridin-6-yl]aniline (100 mg, 59% yield) as a yellow solid.

LCMS (ES, m/z): [M+H]⁺: 414.

### Synthesis of 105-c: 5-chloro-N-(2,4-difluoro-3-[5-fluoro-1-[2-(oxan-2-yl)pyrazol-3-yl]imidazo[1,5-a]pyridin-6-yl]phenyl)-2-methoxypyridine-3-sulfonamide

To a stirred solution of 2,4-difluoro-3-[5-fluoro-1-[2-(oxan-2-yl)pyrazol-3-yl]imidazo[1,5-a]pyridin-6-yl]aniline (80 mg, 0.19 mmol, 1 equiv) in pyridine (1 mL) was added 5-chloro-2-methoxypyridine-3-sulfonyl chloride (70 mg, 0.29 mmol, 1.5 equiv) in portions at room temperature. The reaction was stirred for 2 h, then diluted with water (5 mL). The resulting mixture was extracted with EtOAc (3×20 mL), and the combined organic layers were washed with water and brine, dried over anhydrous Na₂SO₄ and concentrated under reduced pressure. The residue was purified by silica gel column chromatography, eluting with EtOAc/PE (0-70%) to afford 5-chloro-N-(2,4-difluoro-3-[5-fluoro-1-[2-(oxan-2-yl)pyrazol-3-yl]imidazo[1,5-a]pyridin-6-yl]phenyl)-2-methoxypyridine-3-sulfonamide (80 mg, 67% yield) as a yellow solid.

LCMS (ES, m/z): [M+H]⁺: 619.

### Synthesis of Compound 105: 5-chloro-N-[2,4-difluoro-3-[5-fluoro-1-(2H-pyrazol-3-yl)imidazo[1,5-a]pyridin-6-yl]phenyl]-2-methoxypyridine-3-sulfonamide

To a stirred 5-chloro-N-(2,4-difluoro-3-[5-fluoro-1-[2-(oxan-2-yl)pyrazol-3-yl]imidazo[1,5-a]pyridin-6-yl]phenyl)-2-methoxypyridine-3-sulfonamide (80 mg, 1 equiv) was added HCl in MeOH (1 mL, 4 M) dropwise at room temperature. The reaction was stirred for 2 h at room temperature and concentrated under vacuum. The residue was basified to pH 8 with saturated NaHCO₃ (aq.) solution. The resulting mixture was extracted with EtOAc (3×50 mL), and the combined organic layers were washed with water and brine, dried over anhydrous Na₂SO₄ and concentrated under reduced pressure. The crude product was purified by Prep-HPLC with the following conditions: Column, Sunfire Prep C18 OBD, 50*250 mm, 5 μm, 10 nm; mobile phase: 15-50% MeCN/0.1% aqueous formic acid; Detector, UV; to afford 5-chloro-N-[2,4-difluoro-3-[5-fluoro-1-(2H-pyrazol-3-yl)imidazo[1,5-a]pyridin-6-yl]phenyl]-2-methoxypyridine-3-sulfonamide (42 mg, 60% yield) as a white solid.

LCMS (ES, m/z): [M+H]⁺: 535.

¹H NMR (300 MHz, DMSO-d₆) δ 12.93 (s, 1H), 10.50 (s, 1H), 8.61 (s, 1H), 8.51 (d, J=2.6 Hz, 1H), 8.08 (d, J=2.6 Hz, 1H), 8.00 (d, J=9.2 Hz, 1H), 7.78 (s, 1H), 7.54-7.40 (m, 1H), 7.29 (td, J=9.1, 1.6 Hz, 1H), 6.88 (t, J=8.1 Hz, 1H), 6.72 (d, J=2.3 Hz, 1H), 3.91 (s, 3H).

### Example 121: Synthesis of: 6-[2-chloro-3-(5-chloro-2-methoxypyridine-3-sulfonamido)-6-fluorophenyl]-N-methylimidazo[1,5-a]pyrazine-1-carboxamide (Compound 106)

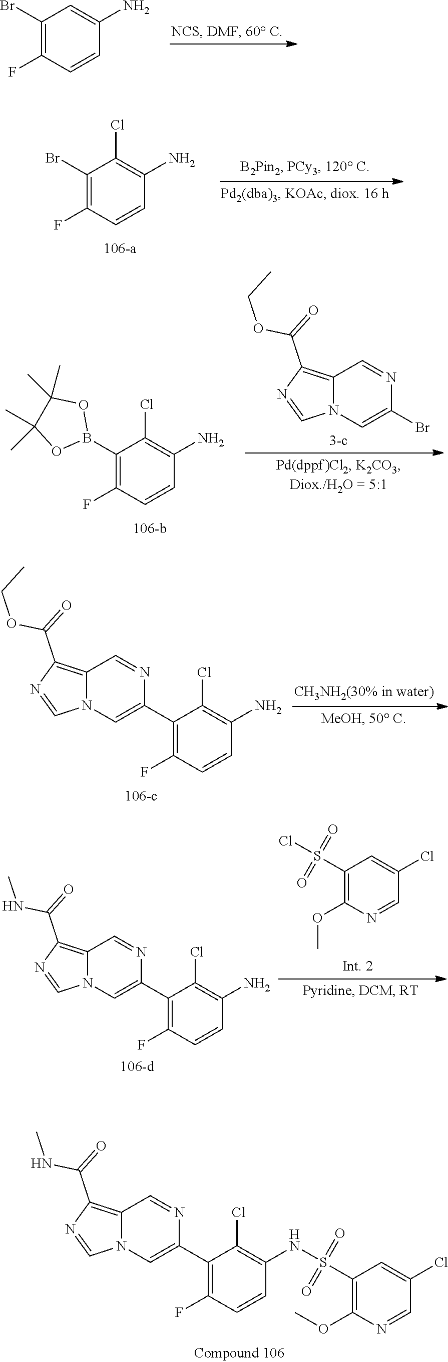

### Synthesis of 106-a: 3-bromo-2-chloro-4-fluoroaniline

Into a 250 mL round-bottom flask was placed 3-bromo-4-fluoroaniline (8 g, 42 mmol, 1 equiv), DMF (100 mL) and NCS (5.6 g, 42 mmol, 1 equiv). The resulting solution was stirred overnight at 60° C. in an oil bath. The reaction mixture was cooled and diluted with 200 mL of water, then extracted with 3×100 mL of ethyl acetate. The organics were washed with 200 ml of water and 200 mL of brine, dried over anhydrous sodium sulfate and concentrated under vacuum. The residue was applied to a silica gel column, eluting with ethyl acetate/petroleum ether (1:10) to give 3-bromo-2-chloro-4-fluoroaniline (4.9 g, 52% yield) as a light brown solid.

### Synthesis of 106-b: 2-chloro-4-fluoro-3-(4,4,5,5-tetramethyl-1,3,2-dioxaborolan-2-yl)aniline

Into a 100 mL round-bottom flask purged and maintained with an inert atmosphere of nitrogen, was placed 3-bromo-2-chloro-4-fluoroaniline (1 g, 4.5 mmol, 1 equiv), dioxane (40 mL), bis(pinacolato)diboron (1.36 g, 5.3 mmol, 1.2 equiv), KOAc (0.87 g, 8.9 mmol, 2 equiv), PCy₃ (0.25 g, 0.9 mmol, 0.2 equiv) and Pd₂(dba)₃ (0.41 g, 0.44 mmol, 0.1 equiv). The resulting solution was stirred for 16 h at 120° C. under nitrogen atmosphere. The reaction mixture was cooled and concentrated under vacuum. The residue was applied to a silica gel column, eluting with ethyl acetate/petroleum ether (1:5) to give 2-chloro-4-fluoro-3-(4,4,5,5-tetramethyl-1,3,2-dioxaborolan-2-yl)aniline (1 g) as a crude, light brown oil.

LCMS (ES, m/z): [M+H]⁺: 272

### Synthesis of 106-c: ethyl 6-(3-amino-2-chloro-6-fluorophenyl)imidazo[1,5-a]pyrazine-1-carboxylate

Into a 40 mL vial purged and maintained with an inert atmosphere of nitrogen, was placed ethyl 6-bromoimidazo[1,5-a]pyrazine-1-carboxylate (280 mg, 1.0 mmol, 1 equiv), dioxane (10 mL), H₂O (2 mL), 2-chloro-4-fluoro-3-(4,4,5,5-tetramethyl-1,3,2-dioxaborolan-2-yl)aniline (338 mg, 1.24 mmol, 1.2 equiv), K₂CO₃ (287 mg, 2.07 mmol, 2 equiv) and Pd(dppf)Cl₂ (76 mg, 0.1 mmol, 0.1 equiv). The reaction was stirred for 3 h at 80° C. under nitrogen atmosphere. The resulting mixture was concentrated under vacuum and the residue was purified by Flash-Prep-HPLC with the following conditions: Column, WelFlash™ C18-I, Spherical C18 20-40 m, 120 g; mobile phase: 10-80% MeCN/0.1% aqueous formic acid; Detector, 220 nm; to give ethyl 6-(3-amino-2-chloro-6-fluorophenyl)imidazo[1,5-a]pyrazine-1-carboxylate (100 mg, 29% yield) as a white solid.

LCMS (ES, m/z): [M+H]⁺: 335

### Synthesis of 106-d: 6-(3-amino-2-chloro-6-fluorophenyl)-N-methylimidazo[1,5-a]pyrazine-1-carboxamide

Into a 40 mL vial, was placed ethyl 6-(3-amino-2-chloro-6-fluorophenyl)imidazo[1,5-a]pyrazine-1-carboxylate (100 mg, 0.3 mmol, 1 equiv), MeOH (3 mL) and methylamine solution (30% in water, 0.5 mL). The reaction was stirred for 8 h at 50° C. in an oil bath, then concentrated under vacuum to give 6-(3-amino-2-chloro-6-fluorophenyl)-N-methylimidazo[1,5-a]pyrazine-1-carboxamide (80 mg, crude) as a white solid which was used in the next step without further purification.

LCMS (ES, m/z): [M+H]⁺: 320

### Synthesis of Compound 106: 6-[2-chloro-3-(5-chloro-2-methoxypyridine-3-sulfonamido)-6-fluorophenyl]-N-methylimidazo[1,5-a]pyrazine-1-carboxamide

Into a 40 mL vial, was placed 6-(3-amino-2-chloro-6-fluorophenyl)-N-methylimidazo[1,5-a]pyrazine-1-carboxamide (50 mg, 0.15 mmol, 1 equiv), pyridine (3 mL) and 5-chloro-2-methoxypyridine-3-sulfonyl chloride (56 mg, 0.23 mmol, 1.5 equiv). The resulting solution was stirred overnight and concentrated under reduced pressure. The residue was purified by Prep-HPLC with the following conditions: Column, welch Vltimate XB-C18, 50×250 mm, 10 μm; Mobile Phase: 20-60% MeCN/0.1% aqueous formic acid; Detector, 220 nm; to give 6-[2-chloro-3-(5-chloro-2-methoxypyridine-3-sulfonamido)-6-fluorophenyl]-N-methylimidazo[1,5-a]pyrazine-1-carboxamide (24 mg, 29% yield) as a white solid.

LCMS (ES, m/z): [M+H]⁺: 525

¹H NMR (300 MHz, DMSO-d₆) δ 10.42 (s, 1H), 9.51 (d, J=1.7 Hz, 1H), 8.63 (d, J=1.7 Hz, 2H), 8.45 (dd, J=19.7, 3.8 Hz, 2H), 8.07 (d, J=2.6 Hz, 1H), 7.50 (dd, J=9.0, 5.6 Hz, 1H), 7.39 (t, J=8.8 Hz, 1H), 3.88 (s, 3H), 2.84 (d, J=4.7 Hz, 3H).

### Example 122: Synthesis of N-[2,4-difluoro-3-[5-fluoro-1-(1H-imidazol-2-yl)imidazo[1,5-a]pyridin-6-yl]phenyl]-5-fluoro-2-methoxypyridine-3-sulfonamide (Compound 107)

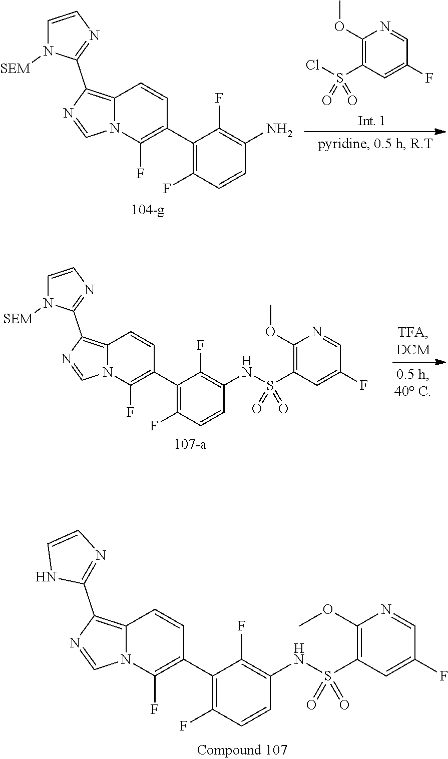 [SMILES: C[Si](C)(C)CCOCn1ccnc1-c1ncn2c(F)c(-c3c(F)ccc(N)c3F)ccc12 ; COc1ncc(F)cc1S(=O)(=O)Cl ; COc1ncc(F)cc1S(=O)(=O)Nc1ccc(F)c(-c2ccc3c(-c4nccn4COCC[Si](C)(C)C)ncn3c2F)c1F ; COc1ncc(F)cc1S(=O)(=O)Nc1ccc(F)c(-c2ccc3c(-c4ncc[nH]4)ncn3c2F)c1F]

### Synthesis of 107-a: N-[2,4-difluoro-3-[5-fluoro-1-(1-[[2-(trimethylsilyl)ethoxy]methyl]imidazol-2-yl)imidazo[1,5-a]pyridin-6-yl]phenyl]-5-fluoro-2-methoxypyridine-3-sulfonamide

To a stirred mixture of 2,4-difluoro-3-[5-fluoro-1-(1-[[2-(trimethylsilyl)ethoxy]methyl]imidazol-2-yl)imidazo[1,5-a]pyridin-6-yl]aniline (200 mg, 0.435 mmol, 1 equiv) in pyridine (2 mL) was added 5-fluoro-2-methoxypyridine-3-sulfonyl chloride (196 mg, 0.87 mmol, 2 equiv) at room temperature. The reaction was stirred for 30 min then diluted with water (20 mL). The resulting mixture was extracted with EA (3×20 mL), and the combined organics were washed with water (50 mL) and brine (50 mL), dried over anhydrous Na₂SO₄ and concentrated under reduced pressure. The residue was purified by silica gel column chromatography, eluting with PE/EA (1/1) to afford N-[2,4-difluoro-3-[5-fluoro-1-(1-[[2-(trimethylsilyl)ethoxy]methyl]imidazol-2-yl)imidazo[1,5-a]pyridin-6-yl]phenyl]-5-fluoro-2-methoxypyridine-3-sulfonamide (124 mg, 44% yield) as a light yellow solid.

LCMS (ES, m/z): [M+H]⁺: 649.

### Synthesis of Compound 107: N-[2,4-difluoro-3-[5-fluoro-1-(1H-imidazol-2-yl)imidazo[1,5-a]pyridin-6-yl]phenyl]-5-fluoro-2-methoxypyridine-3-sulfonamide

A solution of N-[2,4-difluoro-3-[5-fluoro-1-(1-[[2-(trimethylsilyl)ethoxy]methyl]imidazol-2-yl)imidazo[1,5-a]pyridin-6-yl]phenyl]-5-fluoro-2-methoxypyridine-3-sulfonamide (100 mg, 0.15 mmol, 1 equiv) in DCM (1 mL) and TFA (0.5 mL) was stirred for 30 min at 40° C. The mixture was allowed to cool and was concentrated under vacuum. The crude product was purified by Prep-HPLC with the following conditions: Column: Sunfire Prep C18 OBD, 50*250 mm, 5 μm, 10 nm; Mobile Phase: 5-45% 1:1 MeOH:MeCN/0.05% aqueous ammonia; Flow rate: 90 mL/min; to afford N-[2,4-difluoro-3-[5-fluoro-1-(1H-imidazol-2-yl)imidazo[1,5-a]pyridin-6-yl]phenyl]-5-fluoro-2-methoxypyridine-3-sulfonamide (47 mg, 59% yield) as a white solid.

LCMS (ES, m/z): [M+H]⁺: 519.

¹H NMR (300 MHz, DMSO-d₆): δ 12.51 (s, 1H), 10.45 (s, 1H), 8.69 (s, 1H), 8.44 (d, J=3.0 Hz, 1H), 8.12 (d, J=9.3 Hz, 1H), 8.01 (dd, J=7.4, 3.0 Hz, 1H), 7.48-7.40 (m, 1H), 7.24 (t, J=9.0 Hz, 1H), 7.14-7.08 (m, 2H), 6.95 (t, J=8.2 Hz, 1H), 3.89 (s, 3H).

### Example 123: Synthesis of N-[2,4-difluoro-3-[5-fluoro-1-(1H-imidazol-2-yl)imidazo[1,5-a]pyridin-6-yl]phenyl]-5-fluoro-2-methylpyridine-3-sulfonamide (Compound 108)

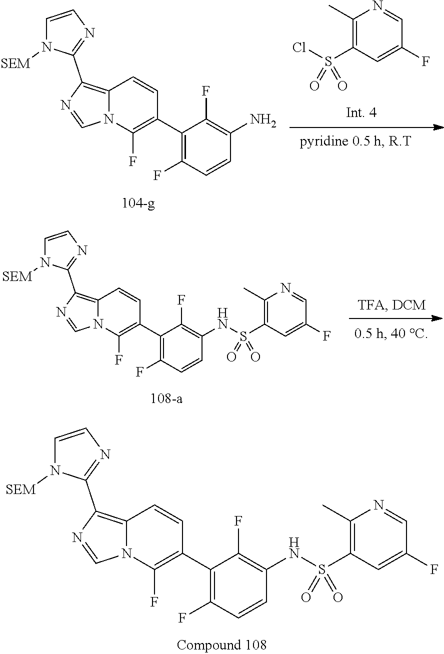 [SMILES: C[Si](C)(C)CCOCn1ccnc1-c1ncn2c(F)c(-c3c(F)ccc(N)c3F)ccc12 ; Cc1ncc(F)cc1S(=O)(=O)Cl ; Cc1ncc(F)cc1S(=O)(=O)Nc1ccc(F)c(-c2ccc3c(-c4nccn4COCC[Si](C)(C)C)ncn3c2F)c1F ; Cc1ncc(F)cc1S(=O)(=O)Nc1ccc(F)c(-c2ccc3c(-c4nccn4COCC[Si](C)(C)C)ncn3c2F)c1F]

### Synthesis of 108-a: N-[2,4-difluoro-3-[5-fluoro-1-(1-[[2-(trimethylsilyl)ethoxy]methyl]imidazol-2-yl) imidazo[1,5-a]pyridin-6-yl]phenyl]-5-fluoro-2-methylpyridine-3-sulfonamide

To a stirred mixture of 2,4-difluoro-3-[5-fluoro-1-(1-[[2-(trimethylsilyl)ethoxy]methyl]imidazol-2-yl)imidazo[1,5-a]pyridin-6-yl]aniline (200 mg, 0.44 mmol, 1 equiv) in pyridine (2 mL) was added 5-fluoro-2-methylpyridine-3-sulfonyl chloride (182 mg, 0.87 mmol, 2 equiv) at room temperature. The reaction was stirred for 30 min at room temperature, then diluted with water (20 mL). The resulting mixture was extracted with EA (3×20 mL), and the combined organics were washed with water (50 mL) and brine (50 mL), dried over anhydrous Na₂SO₄ and concentrated under reduced pressure. The residue was purified by silica gel column chromatography, eluting with PE/EA (1/1) to afford N-[2,4-difluoro-3-[5-fluoro-1-(1-[[2-(trimethylsilyl)ethoxy]methyl]imidazol-2-yl)imidazo[1,5-a]pyridin-6-yl]phenyl]-5-fluoro-2-methylpyridine-3-sulfonamide (109 mg, 40% yield) as a light yellow solid.

LCMS (ES, m/z): [M+H]⁺: 633.

### Synthesis of Compound 108: N-[2,4-difluoro-3-[5-fluoro-1-(1H-imidazol-2-yl)imidazo[1,5-a]pyridin-6-yl]phenyl]-5-fluoro-2-methylpyridine-3-sulfonamide

A solution of N-[2,4-difluoro-3-[5-fluoro-1-(1-[[2-(trimethylsilyl)ethoxy]methyl]imidazol-2-yl)imidazo[1,5-a]pyridin-6-yl]phenyl]-5-fluoro-2-methylpyridine-3-sulfonamide (85 mg, 0.13 mmol, 1 equiv) in DCM (1 mL) and TFA (0.5 mL) was stirred for 30 min at 40° C. The mixture was allowed to cool and was concentrated under vacuum. The crude product was purified by Prep-HPLC with the following conditions: Column: Sunfire Prep C18 OBD, 50*250 mm, 5 μm, 10 nm; Mobile Phase: 5-45% 1:1 MeOH:ACN/0.05% aqueous ammonia; Flow rate: 90 mL/min; to afford N-[2,4-difluoro-3-[5-fluoro-1-(1H-imidazol-2-yl)imidazo[1,5-a]pyridin-6-yl]phenyl]-5-fluoro-2-methylpyridine-3-sulfonamide (48 mg, 71% yield) as a white solid.

LCMS (ES, m/z): [M+H]⁺: 503.

¹H NMR (300 MHz, DMSO-d₆): H NMR (300 MHz, DMSO-d₆) δ 11.65 (br, 1H), 8.74-8.69 (m, 2H), 8.11 (d, J=9.2 Hz, 1H), 7.93 (dt, J=8.4, 1.9 Hz, 1H), 7.45 (q, J=8.6 Hz, 1H), 7.28 (t, J=9.1 Hz, 1H), 7.12 (s, 2H), 6.93 (t, J=8.1 Hz, 1H), 2.78 (s, 3H).

### Example 124: Synthesis of 5-chloro-N-[2,4-difluoro-3-[5-fluoro-1-(1H-imidazol-2-yl)imidazo[1,5-a]pyridine-6-yl]phenyl]-2-methylpyridine-3-sulfonamide (Compound 109)

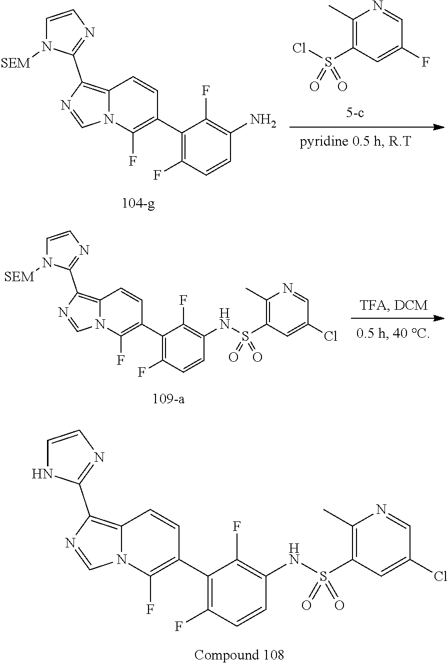 [SMILES: C[Si](C)(C)CCOCn1ccnc1-c1ncn2c(F)c(-c3c(F)ccc(N)c3F)ccc12 ; Cc1ncc(F)cc1S(=O)(=O)Cl ; Cc1ncc(Cl)cc1S(=O)(=O)Nc1ccc(F)c(-c2ccc3c(-c4nccn4COCC[Si](C)(C)C)ncn3c2F)c1F ; Cc1ncc(Cl)cc1S(=O)(=O)Nc1ccc(F)c(-c2ccc3c(-c4ncc[nH]4)ncn3c2F)c1F]

### Synthesis of 109-a: 5-chloro-N-[2,4-difluoro-3-[5-fluoro-1-(1-[[2-(trimethylsilyl)ethoxy]methyl]imidazol-2-yl)imidazo[1,5-a]pyridin-6-yl]phenyl]-2-methylpyridine-3-sulfonamide

To a stirred mixture of 2,4-difluoro-3-[5-fluoro-1-(1-[[2-(trimethylsilyl)ethoxy]methyl]imidazol-2-yl)imidazo[1,5-a]pyridin-6-yl]aniline (200 mg, 0.44 mmol, 1 equiv) in pyridine (2 mL) was added 5-chloro-2-methylpyridine-3-sulfonyl chloride (197 mg, 0.87 mmol, 2 equiv) at room temperature. The reaction was stirred for 30 min at room temperature, then diluted with water (20 mL). The resulting mixture was extracted with EA (3×20 mL), and the combined organic layers were washed with water (50 mL) and brine (50 mL), dried over anhydrous Na₂SO₄ and concentrated under reduced pressure. The residue was purified by silica gel column chromatography, eluting with PE/EA (1/1) to afford 5-chloro-N-[2,4-difluoro-3-[5-fluoro-1-(1-[[2-(trimethylsilyl)ethoxy]methyl]imidazol-2-yl)imidazo[1,5-a]pyridin-6-yl]phenyl]-2-methylpyridine-3-sulfonamide (113 mg, 40% yield) as a light yellow solid.

LCMS (ES, m/z): [M+H]⁺: 649.

### Synthesis of Compound 109: 5-chloro-N-[2,4-difluoro-3-[5-fluoro-1-(1H-imidazol-2-yl)imidazo[1,5-a]pyridine-6-yl]phenyl]-2-methylpyridine-3-sulfonamide

A solution of 5-chloro-N-[2,4-difluoro-3-[5-fluoro-1-(1-[[2-(trimethylsilyl)ethoxy]methyl]imidazol-2-yl)imidazo[1,5-a]pyridin-6-yl]phenyl]-2-methylpyridine-3-sulfonamide (90 mg, 0.139 mmol, 1 equiv) in DCM (1 mL) and TFA (0.5 mL) was stirred for 30 min at 40° C. The mixture was cooled and concentrated under vacuum. The crude product was purified by Prep-HPLC with the following conditions: Column: Sunfire Prep C18 OBD, 50*250 mm, 5 μm, 10 nm; Mobile Phase: 5-45% 1:1 MeOH:MeCN/0.05% aqueous ammonia; Flow rate: 90 mL/min; to afford 5-chloro-N-[2,4-difluoro-3-[5-fluoro-1-(1H-imidazol-2-yl)imidazo[1,5-a]pyridin-6-yl]phenyl]-2-methylpyridine-3-sulfonamide (58 mg, 81% yield) as a white solid.

LCMS (ES, m/z): [M+H]⁺: 519.

¹H NMR (300 MHz, DMSO-d₆): δ 11.87 (s, 1H), 8.75-8.74 (m, 1H), 8.69 (s, 1H), 8.13-8.06 (m, 2H), 7.44 (td, J=9.0, 5.9 Hz, 1H), 7.33-7.20 (m, 1H), 7.12 (s, 2H), 6.93 (t, J=8.2 Hz, 1H), 2.77 (s, 3H).

### Example 125: Synthesis of N-[3-[1-(1H-1,3-benzodiazol-2-yl)imidazo[1,5-a]pyrazin-6-yl]-2,4-difluorophenyl]-5-chloro-2-methoxypyridine-3-sulfonamide (Compound 110)

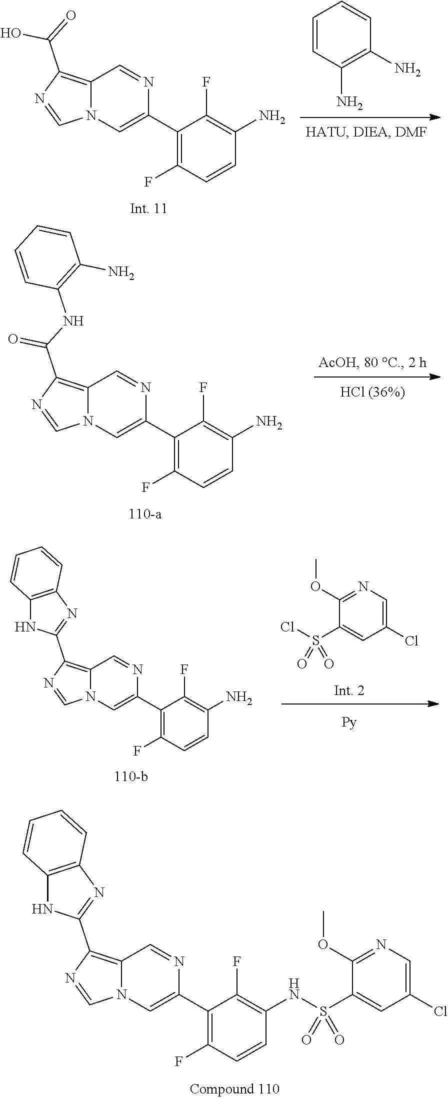 [SMILES: Nc1ccc(F)c(-c2cn3cnc(C(=O)O)c3cn2)c1F ; Nc1ccccc1N ; Nc1ccccc1NC(=O)c1ncn2cc(-c3c(F)ccc(N)c3F)ncc12 ; Nc1ccc(F)c(-c2cn3cnc(-c4nc5ccccc5[nH]4)c3cn2)c1F ; COc1ncc(Cl)cc1S(=O)(=O)Cl ; COc1ncc(Cl)cc1S(=O)(=O)Nc1ccc(F)c(-c2cn3cnc(-c4nc5ccccc5[nH]4)c3cn2)c1F]

### Synthesis of 110-a: 6-(3-amino-2,6-difluorophenyl)-N-(2-aminophenyl)imidazo[1,5-a]pyrazine-1-carboxamide

Into a 40 mL vial were added 6-(3-amino-2,6-difluorophenyl)imidazo[1,5-a]pyrazine-1-carboxylic acid (1.2 g, 4.1 mmol, 1 equiv), HATU (1.73 g, 4.6 mmol, 1.1 equiv), DIEA (1.66 g, 13 mmol, 3 equiv) and DMF (12 mL). The resulting mixture was stirred for 30 min at room temperature. To a stirred solution of o-phenylenediamine (0.46 g, 0.45 mmol, 1 equiv) in DMF (12 mL) was added the activated acid solution, dropwise. The reaction was stirred for 2 h, then diluted with water (100 mL). The resulting mixture was extracted with EtOAc (3×100 mL), and the combined organics were washed with brine (3×50 mL), dried over anhydrous Na₂SO₄ and concentrated under reduced pressure. The residue was purified by silica gel column chromatography, eluting with PE/EtOAc (1:1) to afford 6-(3-amino-2,6-difluorophenyl)-N-(2-aminophenyl)imidazo[1,5-a]pyrazine-1-carboxamide (840 mg, 53% yield) as an off-white solid.

LCMS (ES, m/z): [M+H]⁺: 381.

### Synthesis of 110-b: 3-[1-(1H-1,3-benzodiazol-2-yl)imidazo[1,5-a]pyrazin-6-yl]-2,4-difluoroaniline

Into a 40 mL vial were added 6-(3-amino-2,6-difluorophenyl)-N-(2-aminophenyl)imidazo[1,5-a]pyrazine-1-carboxamide (830 mg, 2.2 mmol, 1 equiv) and AcOH (8 mL). The resulting mixture was stirred for 2 h at 80° C., then cooled and diluted with water (200 mL). The precipitated solids were collected by filtration and washed with water (3×10 mL), then dried. This resulted in the mixture of 3-[1-(1H-1,3-benzodiazol-2-yl)imidazo[1,5-a]pyrazin-6-yl]-2,4-difluoroaniline and N-[3-[1-(1H-1,3-benzodiazol-2-yl)imidazo[1,5-a]pyrazin-6-yl]-2,4-difluorophenyl]acetamide (710 mg) as an off-white solid. This was treated with 36% aqueous HCl (7 mL), stirring for 2 h at 100° C. The resulting mixture was concentrated under vacuum and diluted with water (100 mL). The mixture was adjusted to pH 7 with saturated NaHCO₃ (aq.) and extracted with EtOAc (3×100 mL). The combined organics were washed with brine (3×100 mL), dried over anhydrous Na₂SO₄ and concentrated under reduced pressure. The residue was purified by silica gel column chromatography, eluting with PE/THF (1:1) to afford 3-[1-(1H-1,3-benzodiazol-2-yl)imidazo[1,5-a]pyrazin-6-yl]-2,4-difluoroaniline (590 mg, 75% yield) as a light yellow solid.

LCMS (ES, m/z): [M+H]⁺: 363.

### Synthesis of Compound 110: N-[3-[1-(1H-1,3-benzodiazol-2-yl)imidazo[1,5-a]pyrazin-6-yl]-2,4-difluorophenyl]-5-chloro-2-methoxypyridine-3-sulfonamide

Into an 8 mL vial were added 3-[1-(1H-1,3-benzodiazol-2-yl)imidazo[1,5-a]pyrazin-6-yl]-2,4-difluoroaniline (50 mg, 0.14 mmol, 1 equiv), 5-chloro-2-methoxypyridine-3-sulfonyl chloride (66 mg, 0.27 mmol, 2 equiv) and pyridine (1 mL). The reaction was stirred for 2 h at room temperature, then concentrated. The residue was purified by silica gel column chromatography, eluting with PE/THF (1:1) to give N-[3-[1-(1H-1,3-benzodiazol-2-yl)imidazo[1,5-a]pyrazin-6-yl]-2,4-difluorophenyl]-5-chloro-2-methoxypyridine-3-sulfonamide (24 mg, 31% yield) as a yellow solid.

LCMS (ES, m/z): [M+H]⁺: 568.

¹H NMR (300 MHz, DMSO-d₆) δ 13.05 (s, 1H), 10.47 (s, 1H), 9.77 (d, J=1.6 Hz, 1H), 8.80 (s, 1H), 8.70 (d, J=1.4 Hz, 1H), 8.52 (d, J=2.5 Hz, 1H), 8.10 (d, J=2.6 Hz, 1H), 7.76-7.63 (m, 1H), 7.61-7.35 (m, 2H), 7.33-7.14 (m, 3H), 3.94 (s, 3H).

### Example 126: Synthesis of N-[3-[1-(1H-1,3-benzodiazol-2-yl)imidazo[1,5-a]pyrazin-6-yl]-2,4-difluorophenyl]-5-fluoro-2-methoxypyridine-3-sulfonamide (Compound 111)

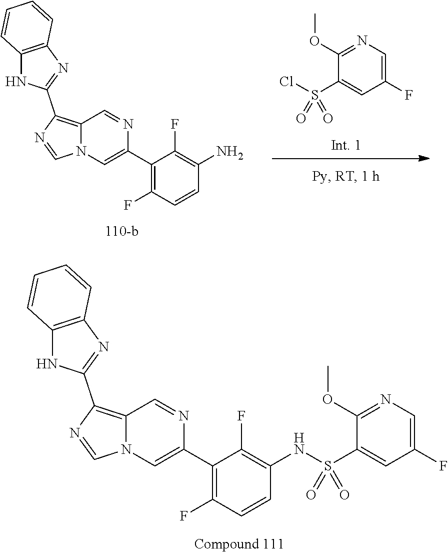 [SMILES: Nc1ccc(F)c(-c2cn3cnc(-c4nc5ccccc5[nH]4)c3cn2)c1F ; COc1ncc(F)cc1S(=O)(=O)Cl ; COc1ncc(F)cc1S(=O)(=O)Nc1ccc(F)c(-c2cn3cnc(-c4nc5ccccc5[nH]4)c3cn2)c1F]

### Synthesis of Compound 111: N-[3-[1-(1H-1,3-benzodiazol-2-yl)imidazo[1,5-a]pyrazin-6-yl]-2,4-difluorophenyl]-5-fluoro-2-methoxypyridine-3-sulfonamide

Into an 8 mL vial were added 3-[1-(1H-1,3-benzodiazol-2-yl)imidazo[1,5-a]pyrazin-6-yl]-2,4-difluoroaniline (60 mg, 0.17 mmol, 1 equiv), 5-fluoro-2-methoxypyridine-3-sulfonyl chloride (74 mg, 0.33 mmol, 2 equiv) and pyridine (1 mL). The resulting mixture was stirred for 2 h at room temperature and concentrated under reduced pressure. The solid was washed with DCM (5 mL), and the residue purified by silica gel column chromatography, eluting with PE/THF (1:1). This resulted in N-[3-[1-(1H-1,3-benzodiazol-2-yl)imidazo[1,5-a]pyrazin-6-yl]-2,4-difluorophenyl]-5-fluoro-2-methoxypyridine-3-sulfonamide (31 mg, 34% yield) as a yellow solid.

LCMS (ES, m/z): [M+H]⁺: 552.

¹H NMR (300 MHz, DMSO-d₆) δ 13.05 (s, 1H), 10.45 (s, 1H), 9.77 (s, 1H), 8.74 (d, J=32.8 Hz, 2H), 8.47 (s, 1H), 8.13-7.95 (m, 1H), 7.78-7.65 (m, 1H), 7.63-7.34 (m, 2H), 7.31-7.16 (m, 3H), 3.93 (s, 3H).

### Example 127: Synthesis of N-[3-[1-(1H-1,3-benzodiazol-2-yl)imidazo[1,5-a]pyrazin-6-yl]-2,4-difluorophenyl]-5-cyano-2-methoxypyridine-3-sulfonamide (Compound 112)

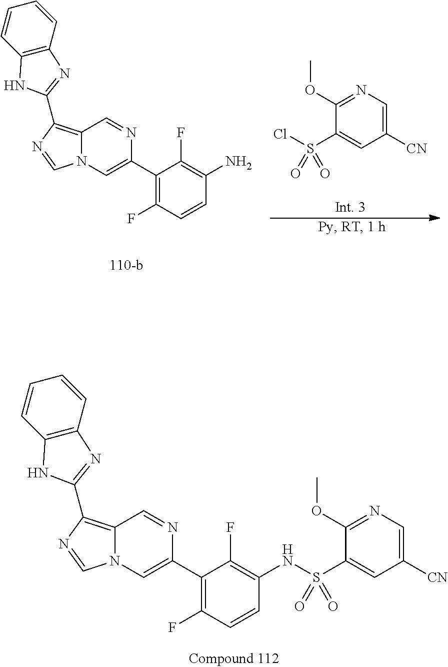 [SMILES: Nc1ccc(F)c(-c2cn3cnc(-c4nc5ccccc5[nH]4)c3cn2)c1F ; COc1ncc(C#N)cc1S(=O)(=O)Cl ; COc1ncc(C#N)cc1S(=O)(=O)Nc1ccc(F)c(-c2cn3cnc(-c4nc5ccccc5[nH]4)c3cn2)c1F]

### Synthesis of Compound 112: N-[3-[1-(1H-1,3-benzodiazol-2-yl)imidazo[1,5-a]pyrazin-6-yl]-2,4-difluorophenyl]-5-cyano-2-methoxypyridine-3-sulfonamide

Into an 8 mL vial were added 3-[1-(1H-1,3-benzodiazol-2-yl)imidazo[1,5-a]pyrazin-6-yl]-2,4-difluoroaniline (60 mg, 0.17 mmol, 1 equiv), 5-cyano-2-methoxypyridine-3-sulfonyl chloride (74 mg, 0.33 mmol, 2 equiv) and pyridine (1 mL). The resulting mixture was stirred for 2 h at room temperature, then concentrated under reduced pressure. The solid was washed with DCM (5 mL), and the residue purified by silica gel column chromatography, eluting with PE/THF (1:1). This resulted in N-[3-[1-(1H-1,3-benzodiazol-2-yl)imidazo[1,5-a]pyrazin-6-yl]-2,4-difluorophenyl]-5-cyano-2-methoxypyridine-3-sulfonamide (38 mg, 41% yield) as a yellow solid.

LCMS (ES, m/z): [M+H]⁺: 559.

¹H NMR (300 MHz, DMSO-d₆) δ 13.05 (s, 1H), 10.56 (s, 1H), 9.77 (d, J=1.6 Hz, 1H), 8.94 (d, J=2.2 Hz, 1H), 8.80 (s, 1H), 8.69 (s, 1H), 8.52 (d, J=2.2 Hz, 1H), 7.71 (dd, J=6.7 Hz, 1H), 7.60-7.36 (m, 2H), 7.35-7.13 (m, 3H), 4.04 (s, 3H).

### Example 128: Synthesis of N-[3-[1-(1H-1,3-benzodiazol-2-yl)imidazo[1,5-a]pyrazin-6-yl]-2,4-difluorophenyl]-5-chloro-2-methylpyridine-3-sulfonamide (Compound 113)

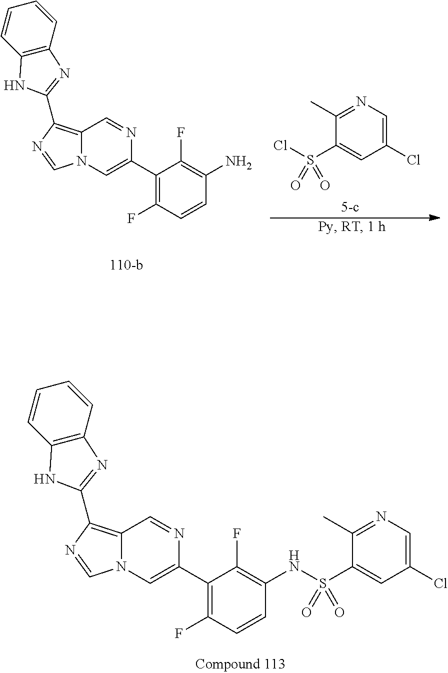 [SMILES: Nc1ccc(F)c(-c2cn3cnc(-c4nc5ccccc5[nH]4)c3cn2)c1F ; Cc1ncc(Cl)cc1S(=O)(=O)Cl ; Cc1ncc(Cl)cc1S(=O)(=O)Nc1ccc(F)c(-c2cn3cnc(-c4nc5ccccc5[nH]4)c3cn2)c1F]

### Synthesis of Compound 113: N-[3-[1-(1H-1,3-benzodiazol-2-yl)imidazo[1,5-a]pyrazin-6-yl]-2,4-difluorophenyl]-5-chloro-2-methylpyridine-3-sulfonamide

Into an 8 mL vial were added 3-[1-(1H-1,3-benzodiazol-2-yl)imidazo[1,5-a]pyrazin-6-yl]-2,4-difluoroaniline (60 mg, 0.17 mmol, 1 equiv), 5-chloro-2-methylpyridine-3-sulfonyl chloride (74 mg, 0.33 mmol, 2 equiv) and pyridine (1 mL). The resulting mixture was stirred for 2 h at room temperature, then concentrated under reduced pressure. The solid was washed with DCM (5 mL) and the residue purified by silica gel column chromatography, eluting with PE/THF (1:1) to give N-[3-[1-(1H-1,3-benzodiazol-2-yl)imidazo[1,5-a]pyrazin-6-yl]-2,4-difluorophenyl]-5-chloro-2-methylpyridine-3-sulfonamide (51 mg, 56% yield) as a yellow solid.

LCMS (ES, m/z): [M+H]⁺: 552.

¹H NMR (300 MHz, DMSO-d₆) δ 13.07 (1H, s), 10.82 (1H, s), 9.76 (1H, m), 8.81 (2H, m), 8.69 (1H, d), 8.10 (1H, d), 7.35-7.80 (3H, m), 7.16-7.34 (3H, m), 2.80 (3H, s).

### Example 129: Synthesis of N-[3-[1-(1H-1,3-benzodiazol-2-yl)imidazo[1,5-a]pyrazin-6-yl]-2,4-difluorophenyl]-5-fluoro-2-methylpyridine-3-sulfonamide (Compound 114)

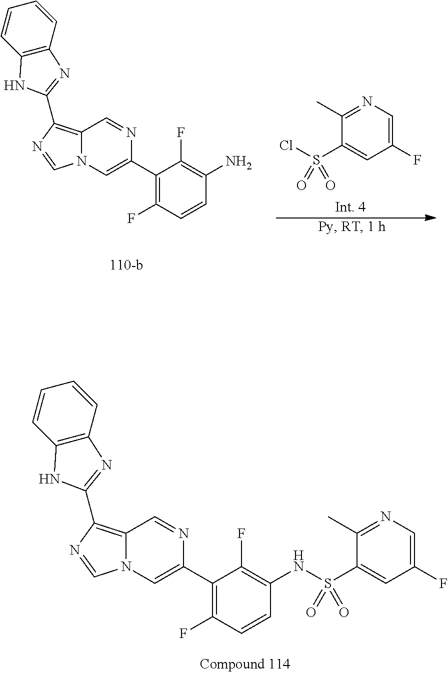 [SMILES: Nc1ccc(F)c(-c2cn3cnc(-c4nc5ccccc5[nH]4)c3cn2)c1F ; Cc1ncc(F)cc1S(=O)(=O)Cl ; Cc1ncc(F)cc1S(=O)(=O)Nc1ccc(F)c(-c2cn3cnc(-c4nc5ccccc5[nH]4)c3cn2)c1F]

### Synthesis of Compound 114: N-[3-[1-(1H-1,3-benzodiazol-2-yl)imidazo[1,5-a]pyrazin-6-yl]-2,4-difluorophenyl]-5-fluoro-2-methylpyridine-3-sulfonamide

Into an 8 mL vial were added 3-[1-(1H-1,3-benzodiazol-2-yl)imidazo[1,5-a]pyrazin-6-yl]-2,4-difluoroaniline (60 mg, 0.17 mmol, 1 equiv), 5-fluoro-2-methylpyridine-3-sulfonyl chloride (69 mg, 0.33 mmol, 2 equiv) and pyridine (1 mL). The resulting mixture was stirred for 2 h at room temperature then concentrated under reduced pressure. The solid was washed with DCM (5 mL), and the residue purified by silica gel column chromatography, eluting with PE/THF (1:1) to give N-[3-[1-(1H-1,3-benzodiazol-2-yl)imidazo[1,5-a]pyrazin-6-yl]-2,4-difluorophenyl]-5-fluoro-2-methylpyridine-3-sulfonamide (45 mg, 51% yield) as a yellow solid.

LCMS (ES, m/z): [M+H]⁺: 536.

¹H NMR (300 MHz, DMSO-d₆) δ 13.05 (s, 1H), 10.81 (s, 1H), 9.76 (d, J=1.6 Hz, 1H), 8.80 (s, 1H), 8.75 (d, J=2.8 Hz, 1H), 8.68 (d, J=1.6 Hz, 1H), 7.97 (dd, J=8.2, 2.9 Hz, 1H), 7.78-7.64 (m, 1H), 7.59-7.48 (m, 1H), 7.42 (td, J=8.8, 5.8 Hz, 1H), 7.33-7.15 (m, 3H), 2.80 (d, J=1.2 Hz, 3H).

### Example 130: Synthesis of N-[3-[1-(4-chloro-1H-imidazol-2-yl)-5-fluoroimidazo[1,5-a]pyridin-6-yl]-2,4-difluorophenyl]-5-fluoro-2-methoxypyridine-3-sulfonamide (Compound 115)

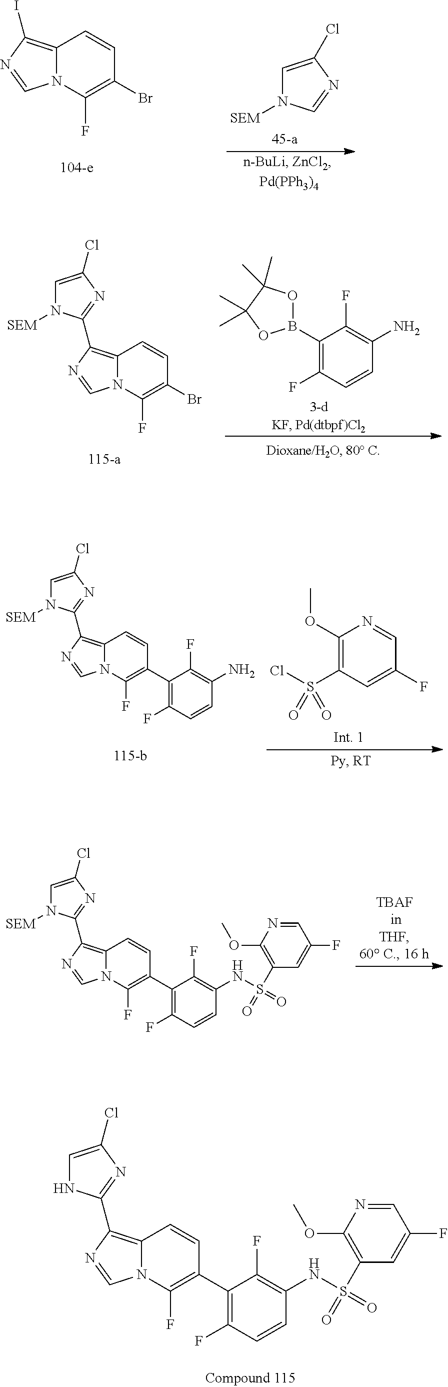

### Synthesis of 115-a: 2-[6-bromo-5-fluoroimidazo[1,5-a]pyridin-1-yl]-4-chloro-1-[[2-(trimethylsilyl)ethoxy]methyl]imidazole

To a stirred solution of 4-chloro-1-[[2-(trimethylsilyl)ethoxy]methyl]imidazole (2.05 g, 8.8 mmol, 6 equiv) in THF (6 mL) was added 2.5 M n-BuLi in hexanes (3.5 mL, 8.8 mmol, 6 equiv) dropwise at −78° C. under N₂ atmosphere. The resulting mixture was stirred for 30 min at low temperature, before 1 M ZnCl₂ in Et₂O (8.8 mL, 8.8 mmol, 6 equiv) was added dropwise at −78° C. The solution was stirred for 30 min at room temperature, then 6-bromo-5-fluoro-1-iodoimidazo[1,5-a]pyridine (500 mg, 1.46 mmol, 1 equiv) and Pd(PPh₃)₄ (339 mg, 0.29 mmol, 0.2 equiv) in THF (4 mL) were added dropwise. The reaction was stirred for 30 min at 60° C., then cooled and quenched by the addition of water (30 mL). The resulting mixture was extracted with EA (3×30 mL), and the combined organics were washed with water (50 mL) and brine (50 mL), dried over anhydrous Na₂SO₄ then concentrated under reduced pressure. The residue was purified by silica gel column chromatography, eluting with EA/PE (0-30%) to afford 2-[6-bromo-5-fluoroimidazo[1,5-a]pyridin-1-yl]-4-chloro-1-[[2-(trimethylsilyl)ethoxy]methyl]imidazole (200 mg, 31% yield) as a yellow solid.

LCMS (ES, m/z): [M+H]⁺: 445, 447.

### Synthesis of 115-b: 3-[I-(4-chloro-1-[[2-(trimethylsilyl)ethoxy]methyl]imidazol-2-yl)-5-fluoroimidazo[1,5-a]pyridin-6-yl]-2,4-difluoroaniline

A solution of 2-[6-bromo-5-fluoroimidazo[1,5-a]pyridin-1-yl]-4-chloro-1-[[2-(trimethylsilyl)ethoxy]methyl]imidazole (220 mg, 0.49 mol, 1 equiv), 2,4-difluoro-3-(4,4,5,5-tetramethyl-1,3,2-dioxaborolan-2-yl)aniline (315 mg, 1.3 mmol, 2.5 equiv), KF (86 mg, 1.5 mmol, 3 equiv) and Pd(dtbpf)Cl₂ (97 mg, 0.15 mmol, 0.3 equiv) in dioxane (2.2 mL) and H₂O (0.2 mL) was stirred for 6 h at 80° C. under nitrogen atmosphere. The mixture was cooled and diluted with water (5 mL). The resulting mixture was extracted with EtOAc (3×20 mL), and the combined organics were washed with water and brine, dried over anhydrous Na₂SO₄ and concentrated under reduced pressure. The residue was purified by silica gel column chromatography, eluting with EtOAc/PE (0-50%) to afford 3-[1-(4-chloro-1-[[2-(trimethylsilyl)ethoxy]methyl]imidazol-2-yl)-5-fluoroimidazo[1,5-a]pyridin-6-yl]-2,4-difluoroaniline (190 mg, 78% yield) as a yellow solid.

LCMS (ES, m/z): [M+H]⁺: 494.

### Synthesis of 115-c: N-[3-[1-(4-chloro-1-[[2-(trimethylsilyl)ethoxy]methyl]imidazol-2-yl)-5-fluoroimidazo[1,5-a]pyridin-6-yl-2,4-difluorophenyl]-5-fluoro-2-methoxypyridine-3-sulfonamide

To a stirred solution of 3-[1-(4-chloro-1-[[2-(trimethylsilyl)ethoxy]methyl]imidazol-2-yl)-5-fluoroimidazo[1,5-a]pyridin-6-yl]-2,4-difluoroaniline (80 mg, 0.16 mmol, 1 equiv) in pyridine (1 mL) was added 5-fluoro-2-methoxypyridine-3-sulfonyl chloride (73 mg, 0.32 mmol, 2 equiv) in portions at room temperature. The reaction was stirred for 2 h at room temperature, then diluted with water (5 mL). The resulting mixture was extracted with EtOAc (3×20 mL). The combined organics were washed with water and brine, dried over anhydrous Na₂SO₄ and concentrated under reduced pressure. The residue was purified by silica gel column chromatography, eluting with EtOAc/PE (0-50%) to afford N-[3-[1-(4-chloro-1-[[2-(trimethylsilyl)ethoxy]methyl]imidazol-2-yl)-5-fluoroimidazo[1,5-a]pyridin-6-yl]-2,4-difluorophenyl]-5-fluoro-2-methoxypyridine-3-sulfonamide (80 mg, 72% yield) as a yellow solid.

LCMS (ES, m/z): [M+H]⁺: 683.

### Synthesis of Compound 115: N-[3-[1-(4-chloro-1H-imidazol-2-yl)-5-fluoroimidazo[1,5-a]pyridin-6-yl]-2,4-difluorophenyl]-5-fluoro-2-methoxypyridine-3-sulfonamide

A solution of N-[3-[1-(4-chloro-1-[[2-(trimethylsilyl)ethoxy]methyl]imidazol-2-yl)-5-fluoroimidazo[1,5-a]pyridin-6-yl]-2,4-difluorophenyl]-5-fluoro-2-methoxypyridine-3-sulfonamide (80 mg, 0.12 mmol, 1 equiv) in 1 M TBAF in THE (0.8 mL, 0.8 mmol, 6.8 equiv) was stirred overnight at 60° C. The mixture was cooled and diluted with water (5 mL).

The resulting mixture was extracted with EtOAc (3×20 mL). The combined organics were washed with sat. NH₄Cl (aq.) and brine, dried over anhydrous Na₂SO₄ and concentrated under reduced pressure. The crude product was purified by Prep-HPLC with the following conditions: Column, XBridge Prep C18 OBD, 5 μm, 19*150 mm; mobile phase: 16-39% MeCN/0.05% aqueous ammonia; Detector, uv; to afford N-[3-[1-(4-chloro-1H-imidazol-2-yl)-5-fluoroimidazo[1,5-a]pyridin-6-yl]-2,4-difluorophenyl]-5-fluoro-2-methoxypyridine-3-sulfonamide (16 mg, 25% yield) as a grey solid.

LCMS (ES, m/z): [M+H]⁺: 553.

¹H NMR (300 MHz, DMSO-d₆) δ 12.89 (s, 1H), 10.48 (s, 1H), 8.72 (s, 1H), 8.45 (d, J=3.0 Hz, 1H), 8.08-7.96 (m, 2H), 7.52-7.38 (m, 1H), 7.25 (d, J=6.8 Hz, 2H), 7.06-6.95 (m, 1H), 3.90 (s, 3H).

### Example 131: Synthesis of 5-chloro-N-[3-[1-(4-chloro-1H-imidazol-2-yl)-5-fluoroimidazo[1,5-a]pyridin-6-yl]-2,4-difluorophenyl-2-methoxypyridine-3-sulfonamide (Compound 116)

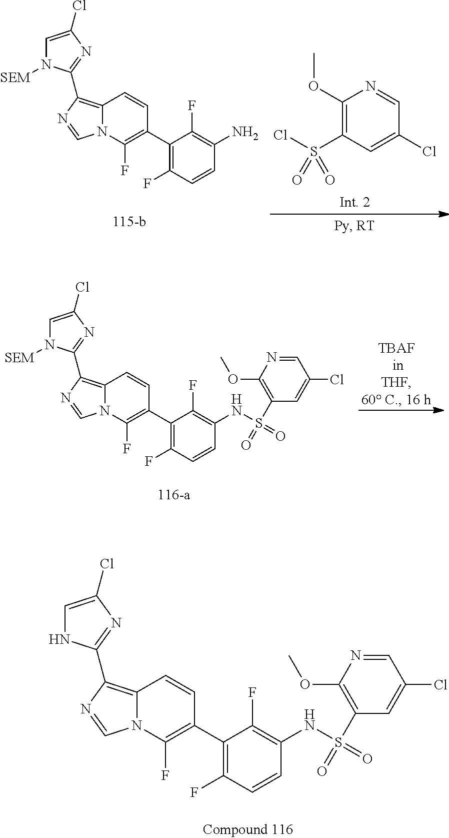 [SMILES: C[Si](C)(C)CCOCn1cc(Cl)nc1-c1ncn2c(F)c(-c3c(F)ccc(N)c3F)ccc12 ; COc1ncc(Cl)cc1S(=O)(=O)Cl ; COc1ncc(Cl)cc1S(=O)(=O)Nc1ccc(F)c(-c2ccc3c(-c4nc(Cl)cn4COCC[Si](C)(C)C)ncn3c2F)c1F ; COc1ncc(Cl)cc1S(=O)(=O)Nc1ccc(F)c(-c2ccc3c(-c4nc(Cl)c[nH]4)ncn3c2F)c1F]

### Synthesis of 116-a: 5-chloro-N-[3-[1-(4-chloro-1-[[2-(trimethylsilyl)ethoxy]methyl]imidazol-2-yl)-5-fluoroimidazo[1,5-a]pyridine-6-yl]-2,4-difluorophenyl]-2-methoxypyridine-3-sulfonamide

To a stirred solution of 3-[1-(4-chloro-1-[[2-(trimethylsilyl)ethoxy]methyl]imidazol-2-yl)-5-fluoroimidazo[1,5-a]pyridin-6-yl]-2,4-difluoroaniline (80 mg, 0.16 mmol, 1 equiv) in pyridine (1 mL) was added 5-chloro-2-methoxypyridine-3-sulfonyl chloride (79 mg, 0.32 mmol, 2 equiv) in portions at room temperature. The reaction was stirred for 2 h then diluted with water (5 mL). The resulting mixture was extracted with EtOAc (3×20 mL). The combined organics were washed with water and brine, dried over anhydrous Na₂SO₄ and concentrated under reduced pressure. The residue was purified by silica gel column chromatography, eluting with EtOAc/PE (0-50%) to afford 5-chloro-N-[3-[1-(4-chloro-1-[[2-(trimethylsilyl)ethoxy]methyl]imidazol-2-yl)-5-fluoroimidazo[1,5-a]pyridine-6-yl]-2,4-difluorophenyl]-2-methoxypyridine-3-sulfonamide (75 mg, 66% yield) as a yellow solid.

LCMS (ES, m/z): [M+H]⁺: 699.

### Synthesis of Compound 116: 5-chloro-N-[3-[1-(4-chloro-1H-imidazol-2-yl)-5-fluoroimidazo[1,5-a]pyridin-6-yl]-2,4-difluorophenyl]-2-methoxypyridine-3-sulfonamide

A solution of 5-chloro-N-[3-[1-(4-chloro-1-[[2-(trimethylsilyl)ethoxy]methyl]imidazol-2-yl)-5-fluoroimidazo[1,5-a]pyridin-6-yl]-2,4-difluorophenyl]-2-methoxypyridine-3-sulfonamide (80 mg, 0.11 mmol, 1 equiv) in TBAF (0.8 mL, 0.8 mmol, 7 equiv) was stirred overnight at 60° C., then cooled and diluted with water (5 mL). The resulting mixture was extracted with EtOAc (3×20 mL). The combined organics were washed with sat. NH₄Cl (aq.) and brine, dried over anhydrous Na₂SO₄ and concentrated under reduced pressure. The crude product was purified by Prep-HPLC with the following conditions: Column, XBridge Prep C18 OBD, 5 μm, 19*150 mm; mobile phase: 19-40% MeCN/0.05% aqueous ammonia; Detector, uv; to give 5-chloro-N-[3-[1-(4-chloro-1H-imidazol-2-yl)-5-fluoroimidazo[1,5-a]pyridin-6-yl]-2,4-difluorophenyl]-2-methoxypyridine-3-sulfonamide (7.6 mg, 12% yield) as an off-white solid.

LCMS (ES, m/z): [M+H]⁺: 569.

¹H NMR (300 MHz, DMSO-d₆) δ 12.90 (s, 1H), 10.51 (s, 1H), 8.72 (s, 1H), 8.51 (d, J=2.6 Hz, 1H), 8.11-7.99 (m, 2H), 7.54-7.40 (m, 1H), 7.35-7.21 (m, 2H), 7.07-6.95 (m, 1H), 3.91 (s, 3H).

### Example 132: Synthesis of 6-[3-(5-chloro-2-methoxypyridine-3-sulfonamido)-2-cyano-6-fluorophenyl]-N-methylimidazo[1,5-a]pyrazine-1-carboxamide (Compound 117)

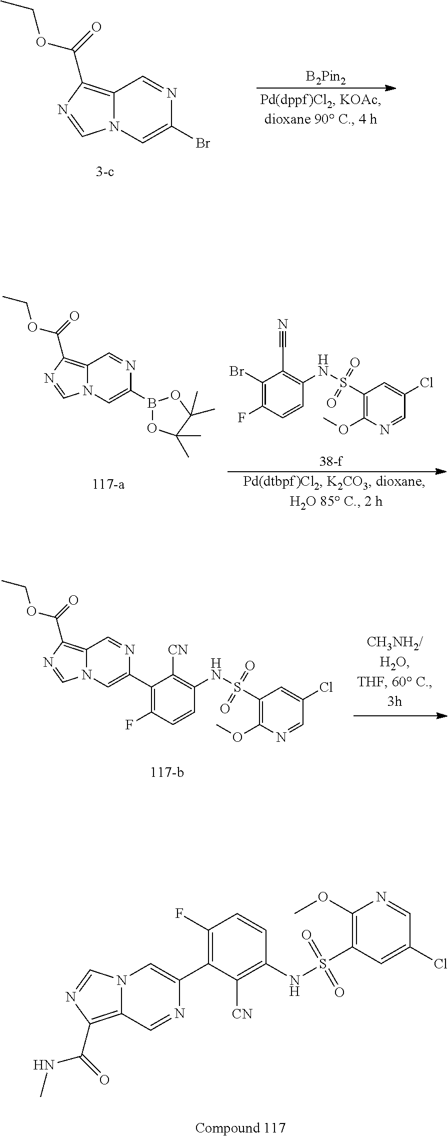

### Synthesis of 117-a: ethyl 6-(4,4,5,5-tetramethyl-1,3,2-dioxaborolan-2-yl)imidazo[1,5-a]pyrazine-1-carboxylate

Into a 25 mL 3-necked round-bottom flask purged and maintained with an inert atmosphere of nitrogen, was placed ethyl 6-bromoimidazo[1,5-a]pyrazine-1-carboxylate (300 mg, 1 mmol, 1 equiv), dioxane (10 mL), bis(pinacolato)diboron (423 mg, 1.7 mmol, 1.5 equiv), Pd(dppf)Cl₂ (81.3 mg, 0.1 mmol, 0.1 equiv) and KOAc (327 mg, 3.3 mmol, 3 equiv). The resulting solution was stirred for 4 h at 90° C. in an oil bath. This solution was used directly in next step.

### Synthesis of 117-b: ethyl 6-[3-(5-chloro-2-methoxypyridine-3-sulfonamido)-2-cyano-6-fluorophenyl]imidazo[1,5-a]pyrazine-1-carboxylate

To the solution from step 1 was added N-(3-bromo-2-cyano-4-fluorophenyl)-5-chloro-2-methoxypyridine-3-sulfonamide (300 mg, 0.72 mmol, 1 equiv), Pd(dtbpf)Cl₂ (47 mg, 0.07 mmol, 0.1 equiv), K₂CO₃ (296 mg, 2.16 mmol, 3 equiv and H₂O (3 mL). The resulting solution was stirred for 4 h at 85° C., then concentrated under vacuum. The residue was applied to a silica gel column, eluting with dichloromethane/methanol (10:1) to give ethyl 6-[3-(5-chloro-2-methoxypyridine-3-sulfonamido)-2-cyano-6-fluorophenyl]imidazo[1,5-a]pyrazine-1-carboxylate (150 mg) as a white solid.

### Synthesis of Compound 117: 6-[3-(5-chloro-2-methoxypyridine-3-sulfonamido)-2-cyano-6-fluorophenyl]-N-methylimidazo[1,5-a]pyrazine-1-carboxamide

Into a 25 mL 3-necked round-bottom flask, was placed 30% CH₃NH₂/H₂O (2 mL), THE (5 mL) and ethyl 6-[3-(5-chloro-2-methoxypyridine-3-sulfonamido)-2-cyano-6-fluorophenyl]imidazo[1,5-a]pyrazine-1-carboxylate (150 mg, 0.28 mmol, 1 equiv). The resulting solution was stirred for 2 h at 50° C. then concentrated under vacuum. The crude product was purified by Flash-Prep-HPLC with the following conditions: Column, Sunfire Prep C18 OBD, 50*250 mm 5 μm 10 nm, Mobile Phase: 5-30% MeCN/0.05% aqueous ammonia; Detector, 220 nm; to give 6-[3-(5-chloro-2-methoxypyridine-3-sulfonamido)-2-cyano-6-fluorophenyl]-N-methylimidazo[1,5-a]pyrazine-1-carboxamide (2 mg) as a white solid.

LCMS (ES, m/z): [M+H]⁺: 516

¹H NMR (300 MHz, DMSO-d₆) δ 10.87 (br s, 1H), 9.53 (d, J=1.7 Hz, 1H), 8.71 (d, J=16.3 Hz, 1H), 8.44 (d, J=4.9 Hz, 1H), 8.36-8.20 (m, 1H), 8.18-8.08 (m, 1H), 8.05 (d, J=2.6 Hz, 1H), 7.52-7.30 (m, 2H), 3.86 (s, 3H), 2.82 (d, J=4.8 Hz, 3H).

### Example 133: Synthesis of N-[2,4-difluoro-3-[1-(1,2-oxazol-5-yl)imidazo[1,5-a]pyridin-6-yl]phenyl]-5-fluoro-2-methoxypyridine-3-sulfonamide (Compound 118)

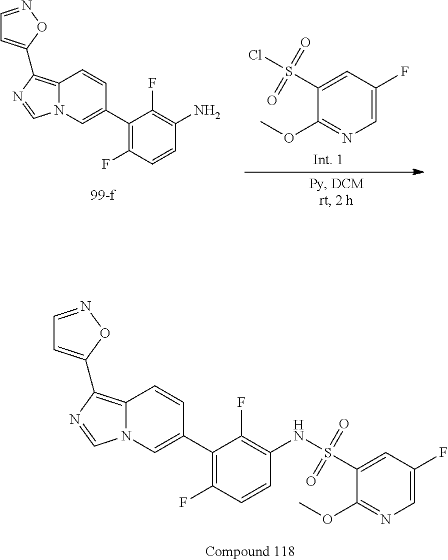 [SMILES: Nc1ccc(F)c(-c2ccc3c(-c4ccno4)ncn3c2)c1F ; COc1ncc(F)cc1S(=O)(=O)Cl ; COc1ncc(F)cc1S(=O)(=O)Nc1ccc(F)c(-c2ccc3c(-c4ccno4)ncn3c2)c1F]

### Synthesis of Compound 118: N-[2,4-difluoro-3-[1-(1,2-oxazol-5-yl)imidazo[1,5-a]pyridin-6-yl]phenyl]-5-fluoro-2-methoxypyridine-3-sulfonamide

To a stirred solution of 2,4-difluoro-3-[1-(1,2-oxazol-5-yl)imidazo[1,5-a]pyridin-6-yl]aniline (100 mg, 0.32 mmol, 1 equiv) and pyridine (76 mg, 0.96 mmol, 3 equiv) in DCM (5 mL) was added 5-fluoro-2-methoxypyridine-3-sulfonyl chloride (108 mg, 0.48 mmol, 1.5 equiv) at room temperature. The reaction was stirred for 1 h at room temperature, then concentrated under reduced pressure. The residue was purified by prep-HPLC with following conditions: Column, welch Vltimate XB-C18, 50×250 mm, 10 m; Mobile Phase: 10-40% MeCN/0.05% aqueous ammonia; Detector, 220 nm; to afford N-[2,4-difluoro-3-[1-(1,2-oxazol-5-yl)imidazo[1,5-a]pyridin-6-yl]phenyl]-5-fluoro-2-methoxypyridine-3-sulfonamide (30 mg, 19% yield) as a white solid.

LCMS (ES, m/z): [M+H]⁺: 502

¹H NMR (300 MHz, DMSO-d₆) δ 10.46 (s, 1H), 8.69-8.62 (m, 3H), 8.48 (d, J=3.0 Hz, 1H), 8.12-7.97 (m, 2H), 7.38 (td, J=8.9, 6.0 Hz, 1H), 7.30-7.21 (m, 1H), 7.06 (dd, J=9.5, 1.5 Hz, 1H), 6.78 (d, J=2.0 Hz, 1H), 3.92 (s, 3H).

### Example 134: Synthesis of 5-cyano-N-[2,4-difluoro-3-[1-(1,2-oxazol-5-yl)imidazo[1,5-a]pyridin-6-yl]phenyl]-2-methoxypyridine-3-sulfonamide (Compound 119)

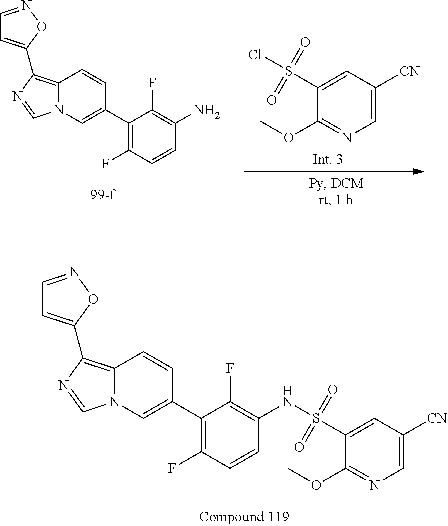 [SMILES: Nc1ccc(F)c(-c2ccc3c(-c4ccno4)ncn3c2)c1F ; COc1ncc(C#N)cc1S(=O)(=O)Cl ; COc1ncc(C#N)cc1S(=O)(=O)Nc1ccc(F)c(-c2ccc3c(-c4ccno4)ncn3c2)c1F]

### Synthesis of Compound 119: 5-cyano-N-[2,4-difluoro-3-[1-(1,2-oxazol-5-yl)imidazo[1,5-a]pyridin-6-yl]phenyl]-2-methoxypyridine-3-sulfonamide

To a stirred solution of 2,4-difluoro-3-[1-(1,2-oxazol-5-yl)imidazo[1,5-a]pyridin-6-yl]aniline (100 mg, 0.32 mmol, 1 equiv) and pyridine (76 mg, 0.96 mmol, 3 equiv) in DCM (5 mL) was added 5-cyano-2-methoxypyridine-3-sulfonyl chloride (111 mg, 0.48 mmol, 1.5 equiv). The resulting mixture was stirred for 1 h then concentrated under reduced pressure. The residue was purified by prep-HPLC with following conditions: Column, Welch Vltimate XB-C18, 50×250 mm, 10 m; Mobile Phase: 5-40% MeCN/0.05% aqueous ammonia; Detector, 220 nm; to afford 5-cyano-N-[2,4-difluoro-3-[1-(1,2-oxazol-5-yl)imidazo[1,5-a]pyridin-6-yl]phenyl]-2-methoxypyridine-3-sulfonamide (30 mg, 18% yield) as a white solid.

LCMS (ES, m/z): [M+H]⁺: 509

¹H NMR (300 MHz, DMSO-d₆) δ 10.56 (s, 1H), 8.95 (d, J=2.2 Hz, 1H), 8.70-8.58 (m, 3H), 8.52 (d, J=2.2 Hz, 1H), 8.07 (d, J=9.5 Hz, 1H), 7.40 (td, J=9.0, 5.9 Hz, 1H), 7.27 (td, J=9.2, 1.5 Hz, 1H), 7.11-6.99 (m, 1H), 6.78 (d, J=1.9 Hz, 1H), 4.03 (s, 3H).

### Example 135: Synthesis of 2-[3-(5-chloro-2-methoxypyridine-3-sulfonamido)-2,6-difluorophenyl]-N-methylimidazo[4,3-b][1,3]thiazole-7-carboxamide (Compound 120)

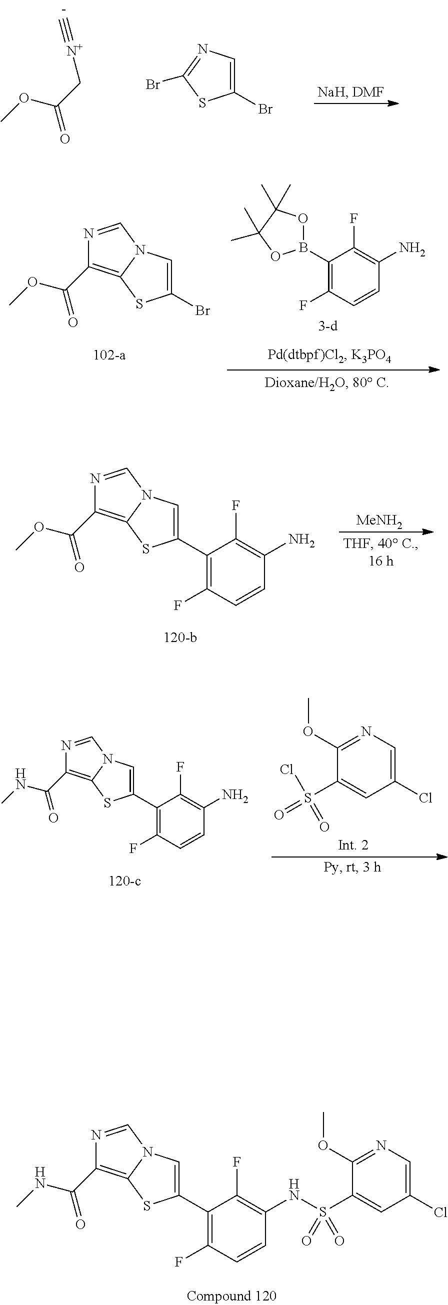

### Synthesis of 120-a: methyl 2-bromoimidazo[4,3-b][1,3]thiazole-7-carboxylate

To a solution of methyl 2-isocyanoacetate (0.82 g, 8 mmol, 2 equiv) in DMF was added sodium hydride (0.2 g, 8 mmol, 2 equiv) at −20° C. The mixture was stirred for 15 min. 2,5-Dibromothiazole (1 g, 4 mmol, 1 equiv) was added and the mixture was allowed to warm to RT and stirred for 3 h. The reaction was quenched by the addition of water/ice (50 mL) at 0° C., then extracted with EtOAc (3×25 mL). The combined organics were washed with brine (2×20 mL), dried over anhydrous Na₂SO₄, and concentrated under reduced pressure. The residue was purified by silica gel column chromatography, eluting with PE/EtOAc (1:1) to afford methyl 2-bromoimidazo[4,3-b][1,3]thiazole-7-carboxylate (500 mg, 47% yield) as a yellow solid.

LCMS (ES, m/z): [M+H]⁺: 261, 263

### Synthesis of 120-b: methyl 2-(3-amino-2,6-difluorophenyl)imidazo[4,3-b][1,3]thiazole-7-carboxylate

To a solution of methyl 2-bromoimidazo[4,3-b][1,3]thiazole-7-carboxylate (580 mg, 2.2 mmol, 1 equiv) and 2,4-difluoro-3-(4,4,5,5-tetramethyl-1,3,2-dioxaborolan-2-yl)aniline (850 mg, 3 mmol, 1.5 equiv) in dioxane (50 mL) and H₂O (10 mL) were added K₃PO₄ (943 mg, 4.4 mmol, 2 equiv) and Pd(dtbpf)Cl₂ (145 mg, 0.2 mmol, 0.1 equiv). After stirring for 1 h at 90° C. under a nitrogen atmosphere, the resulting mixture was concentrated under reduced pressure. The residue was purified by silica gel column chromatography, eluting with PE/EtOAc (5:1) to afford methyl 2-(3-amino-2,6-difluorophenyl)imidazo[4,3-b][1,3]thiazole-7-carboxylate (550 mg, 80% yield) as a yellow solid.

LCMS (ES, m/z): [M+H]⁺: 310

### Synthesis of 120-c: 2-(3-amino-2,6-difluorophenyl)-N-methylimidazo[4,3-b][1,3]thiazole-7-carboxamide

A solution of methyl 2-(3-amino-2,6-difluorophenyl)imidazo[4,3-b][1,3]thiazole-7-carboxylate (550 mg, 1.8 mmol, 1 equiv) and 40% methylamine water solution (5 mL) in THE (5 mL) was stirred for 16 h at 40° C. The resulting mixture was concentrated under vacuum. The residue was purified by prep-HPLC with the following conditions: Column, WelFlash™ C18-I, Spherical C18 20-40 m, 120 g; mobile phase: 20-85% MeCN/0.1% aqueous formic acid; Detector, 220 nm; to give 2-(3-amino-2,6-difluorophenyl)-N-methylimidazo[4,3-b][1,3]thiazole-7-carboxamide (300 mg, 55% yield) as a yellow solid.

LCMS (ES, m/z): [M+H]⁺: 309

### Synthesis of Compound 120: 2-[3-(5-chloro-2-methoxypyridine-3-sulfonamido)-2,6-difluorophenyl]-N-methylimidazo[4,3-bl[1,3]thiazole-7-carboxamide

To a stirred solution of 2-(3-amino-2,6-difluorophenyl)-N-methylimidazo[4,3-b][1,3]thiazole-7-carboxamide (150 mg, 0.49 mmol, 1 equiv) in pyridine (2 mL) was added 5-chloro-2-methoxypyridine-3-sulfonyl chloride (141 mg, 0.59 mmol, 1.2 equiv) dropwise. The resulting mixture was stirred for 3 h then concentrated. The residue was purified by prep-HPLC with the following conditions: Column: welch Vltimate XB-C18, 50×250 mm, 10 μm; Mobile Phase: 20-60% MeCN/0.1% aqueous formic acid; Detector, 220 nm; to give 2-[3-(5-chloro-2-methoxypyridine-3-sulfonamido)-2,6-difluorophenyl]-N-methylimidazo[4,3-b][1,3]thiazole-7-carboxamide (150 mg) as a yellow solid.

LCMS (ES, m/z): [M+H]⁺: 514

¹H NMR (300 MHz, DMSO-d₆) δ 10.54 (s, 1H), 8.52 (d, J=2.6 Hz, 1H), 8.41 (s, 1H), 8.33 (s, 1H), 8.07 (dd, J=9.8, 3.7 Hz, 2H), 7.42 (td, J=8.9, 5.8 Hz, 1H), 7.31 (ddd, J=10.5, 9.0, 1.5 Hz, 1H), 3.92 (s, 3H), 2.77 (d, J=4.7 Hz, 3H).

### Example 136: Synthesis of 5-chloro-N-[2,4-difluoro-3-[1-(1,2-oxazol-5-yl)imidazo[1,5-a]pyridin-6-yl]phenyl]-2-methylpyridine-3-sulfonamide (Compound 121)

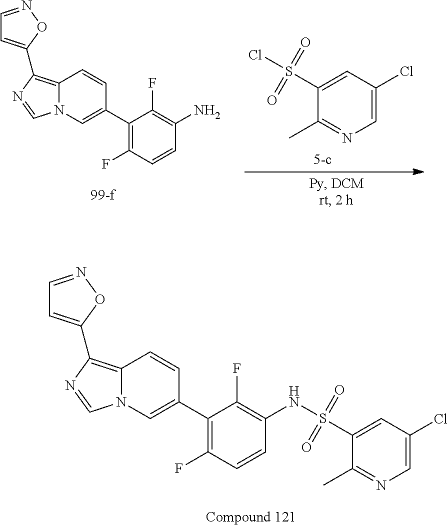 [SMILES: Nc1ccc(F)c(-c2ccc3c(-c4ccno4)ncn3c2)c1F ; Cc1ncc(Cl)cc1S(=O)(=O)Cl ; Cc1ncc(Cl)cc1S(=O)(=O)Nc1ccc(F)c(-c2ccc3c(-c4ccno4)ncn3c2)c1F]

### Synthesis of Compound 121: 5-chloro-N-[2,4-difluoro-3-[1-(1,2-oxazol-5-yl)imidazo[1,5-a]pyridin-6-yl]phenyl]-2-methylpyridine-3-sulfonamide

To a stirred solution of 2,4-difluoro-3-[1-(1,2-oxazol-5-yl)imidazo[1,5-a]pyridin-6-yl]aniline (100 mg, 0.32 mmol, 1 equiv) and pyridine (76 mg, 0.96 mmol, 3 equiv) in DCM (5 mL) was added 5-chloro-2-methylpyridine-3-sulfonyl chloride (109 mg, 0.48 mmol, 1.5 equiv) at room temperature. The resulting mixture was stirred for 2 h at room temperature then concentrated under reduced pressure. The residue was purified by prep-HPLC with following conditions: Column, welch Vltimate XB-C18, 50×250 mm, 10 m; Mobile Phase: 2-47% MeCN/0.05% aqueous ammonia; Detector, 220 nm; to afford 5-chloro-N-[2,4-difluoro-3-[1-(1,2-oxazol-5-yl)imidazo[1,5-a]pyridin-6-yl]phenyl]-2-methylpyridine-3-sulfonamide (20 mg, 13% yield) as a white solid.

LCMS (ES, m/z): [M+H]⁺: 502

¹H NMR (300 MHz, Methanol-d₄) δ 8.64 (d, J=2.4 Hz, 1H), 8.54 (s, 1H), 8.46 (dd, J=4.9, 1.7 Hz, 2H), 8.17 (d, J=2.4 Hz, 1H), 8.11 (d, J=9.5 Hz, 1H), 7.53 (td, J=9.0, 5.7 Hz, 1H), 7.15 (td, J=9.2, 1.8 Hz, 1H), 7.09-7.02 (m, 1H), 6.75 (d, J=2.0 Hz, 1H), 2.85 (s, 3H).

### Example 137: Synthesis of 6-[3-(5-chloro-2-methoxypyridine-3-sulfonamido)-6-fluoro-2-methylphenyl]-N-methylimidazo[1,5-a]pyridine-1-carboxamide (Compound 122)

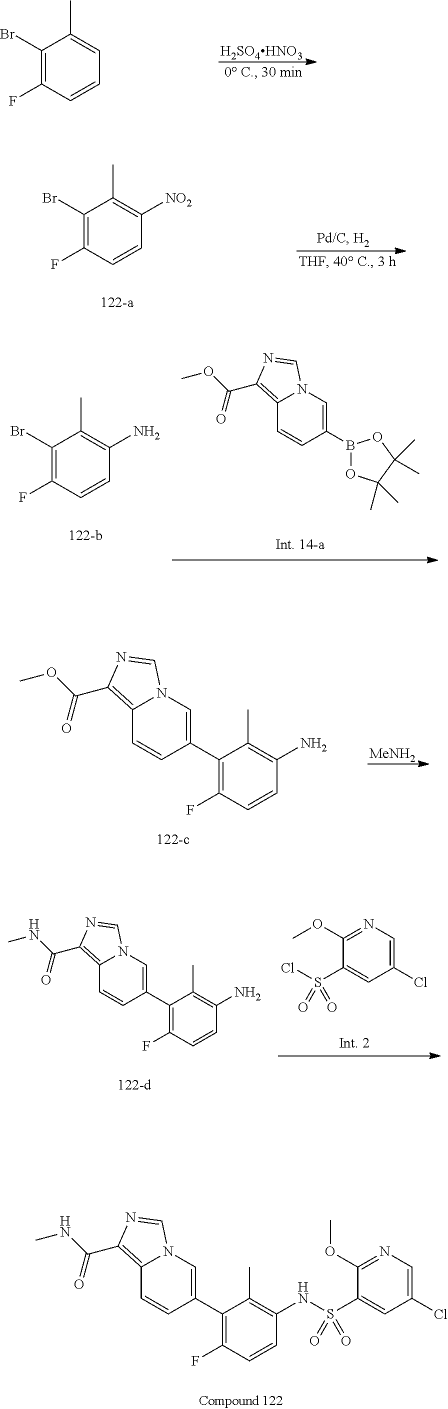

### Synthesis of 122-a: 2-bromo-1-fluoro-3-methyl-4-nitrobenzene

To a stirred solution of 2-bromo-1-fluoro-3-methylbenzene (5 g, 26.4 mmol, 1 equiv) in H₂SO₄ (66 mL) was added HNO₃ (3.3 g, 52 mmol, 2 equiv) dropwise at 0° C. under nitrogen atmosphere. The reaction was stirred for 30 min, then diluted with water (100 mL). The resulting mixture was extracted with EtOAc (2×50 mL), and the combined organics were washed with brine (1×50 mL), dried over anhydrous Na₂SO₄, then concentrated under reduced pressure. The residue was purified by silica gel column chromatography, eluting with PE/EtOAc (50:1) to afford 2-bromo-1-fluoro-3-methyl-4-nitrobenzene (2 g, 32% yield) as an off-white solid.

### Synthesis of 122-b: 3-bromo-4-fluoro-2-methylaniline

To a solution of 2-bromo-1-fluoro-3-methyl-4-nitrobenzene (1 g, 4.3 mmol, 1 equiv) in 10 mL THF was added Pd/C (10%, 200 mg) in a pressure tank. The mixture was hydrogenated at 40° C. under 2 atm hydrogen pressure for 3 h, filtered through a Celite pad and concentrated under reduced pressure to give 3-bromo-4-fluoro-2-methylaniline (500 mg, 57% yield) as a white solid.

LCMS (ES, m/z): [M+H]⁺: 204, 206

### Synthesis of 122-c: 6-(3-amino-6-fluoro-2-methylphenyl)imidazo[1,5-a]pyridine-1-carboxylate

To a solution of methyl 6-(4,4,5,5-tetramethyl-1,3,2-dioxaborolan-2-yl)imidazo[1,5-a]pyridine-1-carboxylate (550 mg, 1.8 mmol, 1 equiv) and 3-bromo-4-fluoro-2-methylaniline (557 mg, 2.7 mmol, 1.5 equiv) in dioxane (10 mL) were added K₃PO₄ (772 mg, 3.6 mmol, 2 equiv) and Pd(dtbpf)Cl₂ (118 mg, 0.18 mmol, 0.1 equiv). After stirring for 3 h at 90° C. under a nitrogen atmosphere, the resulting mixture was concentrated under reduced pressure. The residue was purified by silica gel column chromatography, eluting with PE/EtOAc (1:8) to afford methyl 6-(3-amino-6-fluoro-2-methylphenyl)imidazo[1,5-a]pyridine-1-carboxylate (400 mg, 73% yield) as a brown solid.

LCMS (ES, m/z): [M+H]⁺: 300

### Synthesis of 122-d: 6-(3-amino-6-fluoro-2-methylphenyl)-N-methylimidazo[1,5-a]pyridine-1-carboxamide

To a stirred mixture of methyl 6-(3-amino-6-fluoro-2-methylphenyl)imidazo[1,5-a]pyridine-1-carboxylate (200 mg, 0.67 mmol, 1 equiv) in THE (5 mL) was added 40% aqueous methylamine solution (5 mL) dropwise at room temperature. The resulting mixture was stirred for 16 h at room temperature then concentrated under reduced pressure. The residue was purified by reverse flash chromatography with the following conditions: column, C18 silica gel; mobile phase: 15-75% MeOH/water; detector, UV 254 nm to give 6-(3-amino-6-fluoro-2-methylphenyl)-N-methylimidazo[1,5-a]pyridine-1-carboxamide (180 mg, 90% yield) as a white solid.

LCMS (ES, m/z): [M+H]⁺: 299

### Synthesis of Compound 122: 6-[3-(5-chloro-2-methoxypyridine-3-sulfonamido)-6-fluoro-2-methylphenyl]-N-methylimidazo[1,5-a]pyridine-1-carboxamide

To a stirred solution of 6-(3-amino-6-fluoro-2-methylphenyl)-N-methylimidazo[1,5-a]pyridine-1-carboxamide (80 mg, 0.3 mmol, 1 equiv) in pyridine (2 mL) was added 5-chloro-2-methoxypyridine-3-sulfonyl chloride (78 mg, 0.36 mmol, 1.2 equiv) in DCM (2 mL) dropwise at room temperature. The reaction was stirred for 2 h then concentrated under reduced pressure. The residue was purified by prep-HPLC with the following conditions: Column, welch Vltimate XB-C18, 50×250 mm, 10 μm; Mobile Phase: 25-65% MeCN/0.1% aqueous formic acid; detector, 220 nm; to give 6-[3-(5-chloro-2-methoxypyridine-3-sulfonamido)-6-fluoro-2-methylphenyl]-N-methylimidazo[1,5-a]pyridine-1-carboxamide (30 mg, 22% yield) as a white solid

LCMS (ES, m/z): [M+H]⁺: 504

¹H NMR (300 MHz, DMSO-d₆) δ 10.04 (s, 1H), 8.53 (d, J=2.6 Hz, 1H), 8.49-8.39 (m, 2H), 8.13 (d, J=9.3 Hz, 1H), 8.06 (dd, J=13.6, 3.7 Hz, 2H), 7.15 (t, J=8.8 Hz, 1H), 7.07 (dd, J=8.9, 5.5 Hz, 1H), 6.98-6.88 (m, 1H), 3.97 (s, 3H), 2.81 (d, J=4.7 Hz, 3H), 2.06 (d, J=10.5 Hz, 3H).

### Example 138: Synthesis of 5-cyano-N-[2,4-difluoro-3-[1-(4,5,6,7-tetrahydro-1H-1,3-benzodiazol-2-yl)imidazo[1,5-a]pyridin-6-yl]phenyl]-2-methoxypyridine-3-sulfonamide (Compound 123)

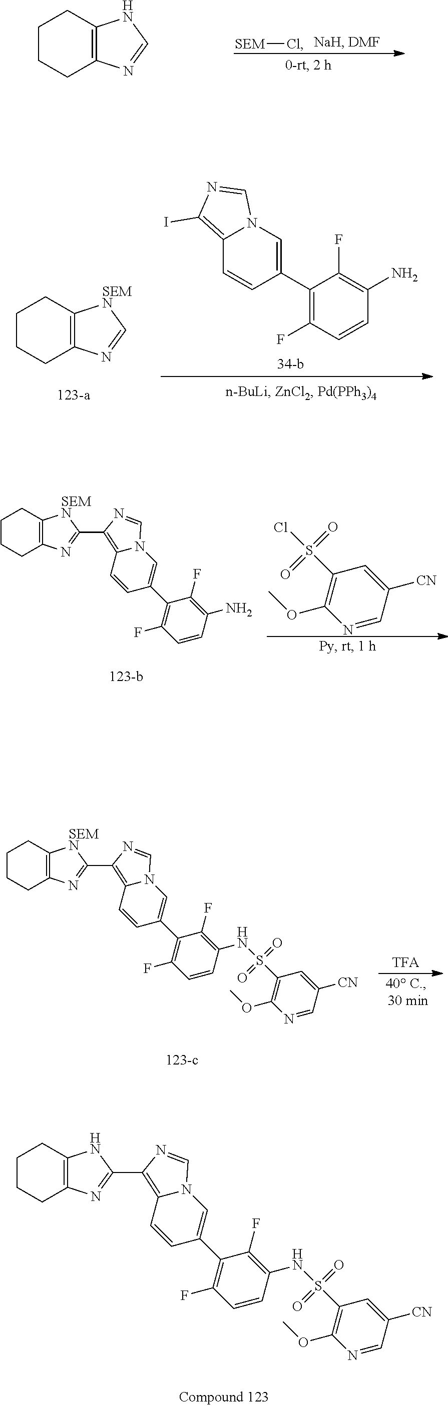

### Synthesis of 123-a: 1-[[2-(trimethylsilyl)ethoxy]methyl]-4,5,6,7-tetrahydro-1,3-benzodiazole

Into a 100 mL 3-necked round-bottom flask were added 4,5,6,7-tetrahydro-1H-1,3-benzodiazole (500 mg, 4.1 mmol, 1 equiv) in DMF (5 mL). 60% NaH in oil (294 mg, 12.2 mmol, 3 equiv) was added in portions at 0° C., and the resulting mixture was stirred for 30 min in the ice bath. To this was added [2-(chloromethoxy)ethyl]trimethylsilane (682 mg, 4.1 mmol, 1 equiv) dropwise at 0° C. The reaction was stirred for 2 h at room temperature, then was quenched with water (100 mL). The resulting mixture was extracted with EtOAc (3×100 mL), and the combined organics were washed with brine (3×50 mL), dried over anhydrous Na₂SO₄ and concentrated under reduced pressure. The residue was purified by silica gel column chromatography, eluting with PE/EtOAc (1:1) to afford 1-[[2-(trimethylsilyl)ethoxy]methyl]-4,5,6,7-tetrahydro-1,3-benzodiazole (800 mg, 77% yield) as a colorless oil.

LCMS (ES, m/z): [M+H]⁺: 253.

### Synthesis of 123-b: 2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]aniline

Into a 40 mL vial was added 1-[[2-(trimethylsilyl)ethoxy]methyl]-4,5,6,7-tetrahydro-1,3-benzodiazole (408 mg, 1.6 mmol, 3 equiv) in THE (5 mL). To this was added 2.5M n-BuLi in hexanes (0.7 mL, 1.6 mmol, 3 equiv) dropwise at −78° C. The resulting mixture was stirred for 1 h at −78° C., before 1 M ZnCl₂ in ether solution (1.6 mL, 1.6 mmol, 1 equiv) was added. This was stirred for 1 h to room temperature, then 2,4-difluoro-3-[1-iodoimidazo[1,5-a]pyridin-6-yl]aniline (200 mg, 0.54 mmol, 1 equiv), Pd(PPh₃)₄ (62 mg, 0.054 mmol, 0.1 equiv) were added. The reaction was stirred for 1 h at 60° C., then was cooled and diluted with water (100 mL). The resulting mixture was extracted with EtOAc (3×100 mL). The combined organics were washed with brine (3×50 mL), dried over anhydrous Na₂SO₄ and concentrated under reduced pressure. The residue was purified by silica gel column chromatography, eluting with PE/THF (1:1) to afford 2,4-difluoro-3-[1-(1-[[2-(trimethylsilyl)ethoxy]methyl]-4,5,6,7-tetrahydro-1,3-benzodiazol-2-yl)imidazo[1,5a]pyridin-6-yl]aniline (120 mg, 33% yield) as a light brown solid.

LCMS (ES, m/z): [M+H]⁺: 496.

### Synthesis of 123-c: 5-cyano-N-[2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]phenyl]-2-methoxypyridine-3-sulfonamide

Into an 8 mL vial were added 2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]aniline (200 mg, 0.4 mmol, 1 equiv), 5-cyano-2-methoxypyridine-3-sulfonyl chloride (188 mg, 0.81 mmol, 2 equiv) and pyridine (1 mL). The resulting mixture was stirred for 2 h at room temperature, then concentrated. The residue was purified by silica gel column chromatography, eluting with PE/THF (1:1) to afford 5-cyano-N-[2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]phenyl]-2-methoxypyridine-3-sulfonamide (210 mg, 58% yield) as a brown solid.

LCMS (ES, m/z): [M+H]⁺: 692.

### Synthesis of Compound 123: 5-cyano-N-[2,4-difluoro-3-[1-(4,5,6,7-tetrahydro-1H-1,3-benzodiazol-2-yl)imidazo[1,5-a]pyridin-6-yl]phenyl]-2-methoxypyridine-3-sulfonamide

Into an 8 mL vial were added 5-cyano-N-[2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]phenyl]-2-methoxypyridine-3-sulfonamide (190 mg, 0.28 mmol, 1 equiv) and TFA (5 mL). The resulting mixture was stirred for 30 min at 40° C., then cooled and concentrated under vacuum. The crude product was purified by Prep-HPLC with the following conditions: Column: Sunfire Prep C18 OBD, 50*250 mm 5 μm 10 nm; Mobile Phase: 18-48% MeCN/0.1% aqueous formic acid; Flow rate: 90 mL/min; detector 220 nm; to afford 5-cyano-N-[2,4-difluoro-3-[1-(4,5,6,7-tetrahydro-1H-1,3-benzodiazol-2-yl)imidazo[1,5-a]pyridin-6-yl]phenyl]-2-methoxypyridine-3-sulfonamide (24 mg, 16% yield) as a yellow solid.

LCMS (ES, m/z): [M+H]⁺: 562.

¹H NMR (300 MHz, DMSO-d₆) δ 8.94 (1H, s), 8.48-8.60 (3H, m), 8.21 (1H, d), 7.20-7.45 (2H, m), 6.88 (1H, d), 4.04 (3H, s), 2.57 (4H, m), 1.80 (4H, m).

### Example 139: Synthesis of 5-chloro-N-[2,4-difluoro-3-[1-(5-methyl-4H-1,2,4-triazol-3-yl)imidazo[1,5-a]pyrazin-6-yl]phenyl]-2-methoxypyridine-3-sulfonamide (Compound 124)

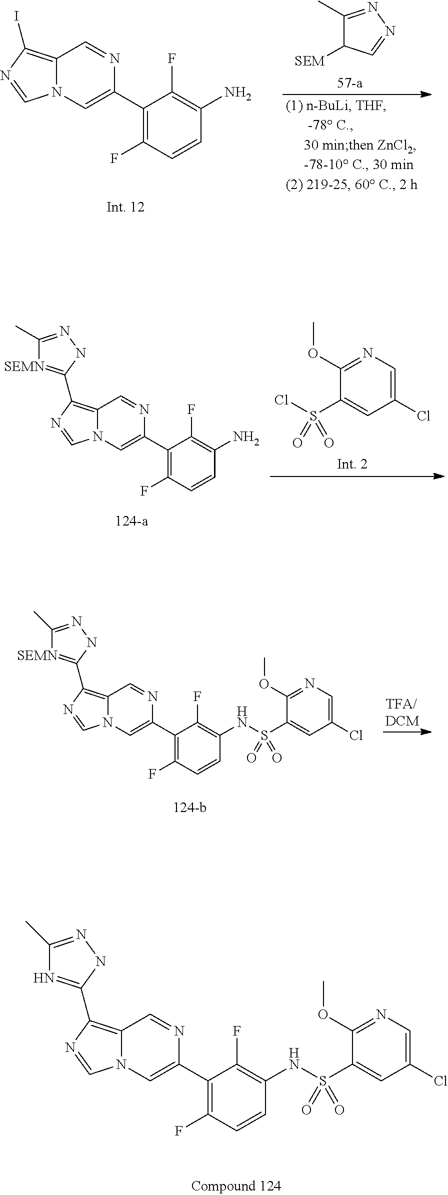

### Synthesis of 124-a: 2,4-difluoro-3-[1-(5-methyl-4-[[2-(trimethylsilyl)ethoxy]methyl]-1,2,4-triazol-3-yl)imidazo[1,5-a]pyrazin-6-yl]aniline

To a solution of 3-methyl-4-[[2-(trimethylsilyl)ethoxy]methyl]-1,2,4-triazole (2 g, 9.4 mmol, 5 equiv) in THF (20 mL) was added dropwise n-butyllithium solution (2.5 M in THF, 3.75 mL, 9.4 mmol) at −78° C. under N₂ atmosphere. The reaction mixture was stirred at −78° C. for 30 min, then a solution of ZnCl₂ (1.0 M in Et₂O, 9.4 mL, 9.4 mmol) was added dropwise and the mixture was stirred for 30 min at room temperature. A solution of Pd(PPh₃)₄ (434 mg, 0.38 mmol, 0.2 equiv) and 2,4-difluoro-3-[1-iodoimidazo[1,5-a]pyrazin-6-yl]aniline (700 mg, 1.88 mmol, 1 equiv) in THF (2 mL) was then added dropwise and the mixture was stirred for 1 h at 60° C. under nitrogen atmosphere. The reaction was cooled and quenched with water/sat. NH₄Cl (50 mL). This was extracted with 1:1 ether/EtOAc (2×50 mL). The combined extracts were washed with brine (50 mL), dried over anhydrous Na₂SO₄ and concentrated. The residue was purified by silica gel column chromatography, eluting with PE/EtOAc (1:1) to afford 2,4-difluoro-3-[1-(5-methyl-4-[[2-(trimethylsilyl)ethoxy]methyl]-1,2,4-triazol-3-yl)imidazo[1,5-a]pyrazin-6-yl]aniline (350 mg, 41% yield) as a yellow solid.

LCMS (ES, m/z): [M+H]⁺: 458

### Synthesis of 124-b: 5-chloro-N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-2-methoxypyridine-3-sulfonamide

To a stirred solution of 2,4-difluoro-3-[1-(5-methyl-4-[[2-(trimethylsilyl)ethoxy]methyl]-1,2,4-triazol-3-yl)imidazo[1,5-a]pyrazin-6-yl]aniline (75 mg, 0.16 mmol, 1 equiv) in pyridine (2 mL) was added 5-chloro-2-methoxypyridine-3-sulfonyl chloride (48 mg, 0.2 mmol, 1.2 equiv) in portions at room temperature. The reaction was stirred for 3 h at room temperature then diluted with MeOH (2 mL) and concentrated. The residue was purified by reverse flash chromatography with the following conditions: column, C18 silica gel; mobile phase: 25-75% MeOH/water; detector, UV 254 nm; to give 5-chloro-N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-2-methoxypyridine-3-sulfonamide (50 mg, 46% yield) as a white solid.

LCMS (ES, m/z): [M+H]⁺: 663

### Synthesis of Compound 124: 5-chloro-N-[2,4-difluoro-3-[1-(5-methyl-4H-1,2,4-triazol-3-yl)imidazo[1,5-a]pyrazin-6-yl]phenyl]-2-methoxypyridine-3-sulfonamide

To a stirred solution of 5-chloro-N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-2-methoxypyridine-3-sulfonamide (50 mg, 0.075 mmol, 1 equiv) in DCM (3 mL) was added TFA (1 mL). The resulting mixture was stirred for 2 h at room temperature then concentrated under reduced pressure. The residue was purified by prep-HPLC with the following conditions: Column, welch Vltimate XB-C18, 50×250 mm, 10 μm; Mobile Phase: 17-53% MeCN/0.1% aqueous formic acid; Detector, 220 nm; to afford 5-chloro-N-[2,4-difluoro-3-[1-(5-methyl-4H-1,2,4-triazol-3-yl)imidazo[1,5-a]pyrazin-6-yl]phenyl]-2-methoxypyridine-3-sulfonamide (25 mg, 62% yield) as a white solid.

LCMS (ES, m/z): [M+H]⁺: 533

¹H NMR (300 MHz, DMSO-d₆) δ 13.79 (s, 1H), 10.45 (s, 1H), 9.55 (d, J=1.8 Hz, 1H), 8.69-8.56 (m, 2H), 8.51 (d, J=2.6 Hz, 1H), 8.09 (d, J=2.6 Hz, 1H), 7.46-7.37 (m, 1H), 7.24 (t, J=9.1 Hz, 1H), 3.93 (s, 3H), 2.47 (s, 3H).

### Example 140: Synthesis of N-[2,4-difluoro-3-[1-(1,2-oxazol-5-yl)imidazo[1,5-a]pyridin-6-yl]phenyl]-5-fluoro-2-methylpyridine-3-sulfonamide (Compound 125)

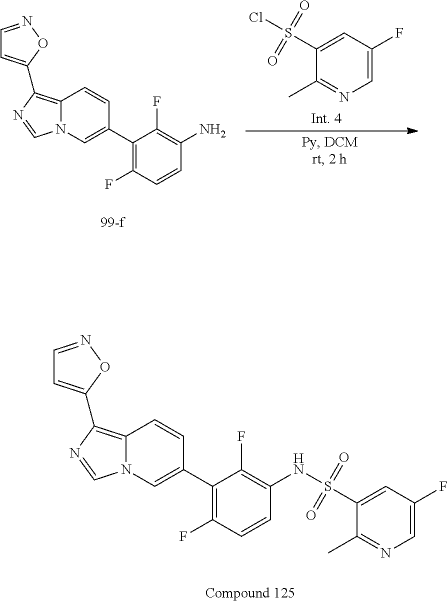 [SMILES: Nc1ccc(F)c(-c2ccc3c(-c4ccno4)ncn3c2)c1F ; Cc1ncc(F)cc1S(=O)(=O)Cl ; Cc1ncc(F)cc1S(=O)(=O)Nc1ccc(F)c(-c2ccc3c(-c4ccno4)ncn3c2)c1F]

### Synthesis of Compound 125: N-[2,4-difluoro-3-[1-(1,2-oxazol-5-yl)imidazo[1,5-a]pyridin-6-yl]phenyl]-5-fluoro-2-methylpyridine-3-sulfonamide

To a stirred solution of 2,4-difluoro-3-[1-(1,2-oxazol-5-yl)imidazo[1,5-a]pyridin-6-yl]aniline (100 mg, 0.3 mmol, 1 equiv) and pyridine (76 mg, 0.96 mmol, 3 equiv) in DCM (5 mL) was added 5-fluoro-2-methylpyridine-3-sulfonyl chloride (100 mg, 0.6 mmol, 1.5 equiv) at room temperature. The resulting mixture was stirred for 1 h, then concentrated under reduced pressure. The residue was purified by prep-HPLC with following conditions: Column, welch Vltimate XB-C18, 50×250 mm, 10 m; Mobile Phase: 5-40% MeCN/0.05% aqueous ammonia; Detector 220 nm; to afford N-[2,4-difluoro-3-[1-(1,2-oxazol-5-yl)imidazo[1,5-a]pyridin-6-yl]phenyl]-5-fluoro-2-methylpyridine-3-sulfonamide (20 mg, 13% yield) as a white solid.

LCMS (ES, m/z): [M+H]⁺: 486

¹H NMR (300 MHz, Methanol-d₄) δ 8.54 (d, J=3.0 Hz, 2H), 8.50-8.43 (m, 2H), 8.11 (d, J=9.5 Hz, 1H), 7.99 (dd, J=8.2, 2.9 Hz, 1H), 7.50 (td, J=9.0, 5.7 Hz, 1H), 7.17-7.02 (m, 2H), 6.75 (d, J=2.0 Hz, 1H), 2.86 (d, J=1.3 Hz, 3H).

### Example 141: Synthesis of 5-cyano-N-[3-[1-(4,5-dimethyl-1H-imidazol-2-yl)imidazo[1,5-a]pyridin-6-yl]-2,4-difluorophenyl]-2-methoxypyridine-3-sulfonamide (Compound 126)

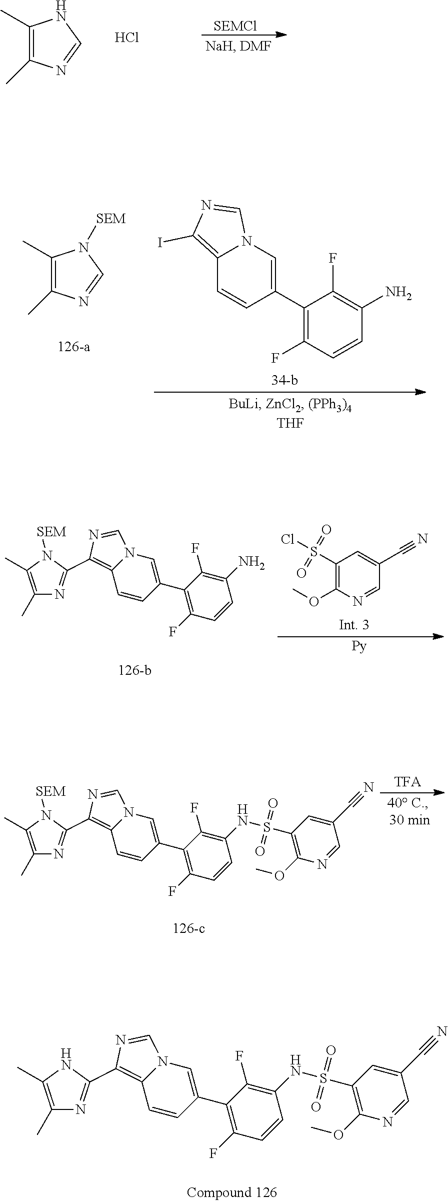

### Synthesis of 126-a: 4,5-dimethyl-1-[[2-(trimethylsilyl)ethoxy]methyl]imidazole

Into a 40 mL vial were added 4,5-dimethyl-1H-imidazole hydrochloride (500 mg, 3.8 mmol, 1 equiv) and DMF (10 mL). The mixture was cooled to −30° C. and 60% NaH (180 mg, 7.5 mmol, 2 equiv) was added in portions. The resulting mixture was stirred for 30 min at −30° C., then[2-(chloromethoxy)ethyl]trimethylsilane (628 mg, 3.8 mmol, 1 equiv) was added dropwise. The mixture was allowed to stir to 0° C. over 2 hrs, then quenched with water (100 mL). This was extracted with EtOAc (3×100 mL) and the combined organics were washed with brine (3×50 mL), dried over anhydrous Na₂SO₄, then concentrated under reduced pressure. The residue was purified by silica gel column chromatography, eluting with PE/THF (1:1) to afford 4,5-dimethyl-1-[[2-(trimethylsilyl)ethoxy]methyl]imidazole (800 mg, 94% yield) as a colorless oil.

LCMS (ES, m/z): [M+H]⁺: 227.

### Synthesis of 126-b: 3-[1-(4,5-dimethyl-1-[[2-(trimethylsilyl)ethoxy]methyl]imidazol-2-yl)imidazo[1,5-a]pyridin-6-yl]-2,4-difluoroaniline

Into a 40 mL vial were added 4,5-dimethyl-1-[[2-(trimethylsilyl)ethoxy]methyl]imidazole (367 mg, 1.6 mmol, 3 equiv) and THE (5 mL). n-BuLi (0.65 mL, 1.6 mmol, 3 equiv) was added dropwise at −78° C. The resulting mixture was stirred for 1 h at −78° C., then 1 M ZnCl₂ solution (1.6 mL, 1.6 mmol, 3 equiv) was added. The mixture was stirred to room temperature over 1 hr. To the resultant mixture was added 2,4-difluoro-3-[1-iodoimidazo[1,5-a]pyridin-6-yl]aniline (200 mg, 0.54 mmol, 1 equiv) and Pd(PPh₃)₄ (62 mg, 0.054 mmol, 0.1 equiv). This was stirred for 30 min at 60° C. under N₂ atmosphere. The resulting mixture was diluted with water (100 mL) and extracted with EtOAc (3×100 mL). The combined organics were washed with brine (3×50 mL), dried over anhydrous Na₂SO₄, and concentrated under reduced pressure. The residue was purified by silica gel column chromatography, eluting with PE/THF (1:1) to afford 3-[1-(4,5-dimethyl-1-[[2-(trimethylsilyl)ethoxy]methyl]imidazol-2-yl)imidazo[1,5-a]pyridin-6-yl]-2,4-difluoroaniline (230 mg, 81% yield) as a brown solid.

LCMS (ES, m/z): [M+H]⁺: 470.

### Synthesis of 126-c: 5-cyano-N-[3-[1-(4,5-dimethyl-1-[[2-(trimethylsilyl)ethoxy]methyl]imidazol-2-yl) imidazo[1,5-a]pyridin-6-yl]-2,4-difluorophenyl]-2-methoxypyridine-3-sulfonamide

Into an 8 mL vial were added 3-[1-(4,5-dimethyl-1-[[2-(trimethylsilyl)ethoxy]methyl]imidazol-2-yl) imidazo[1,5-a]pyridin-6-yl]-2,4-difluoroaniline (180 mg, 0.38 mmol, 1 equiv), 5-cyano-2-methoxypyridine-3-sulfonyl chloride (180 mg, 0.77 mmol, 2 equiv) and pyridine (4 mL). The resulting mixture was stirred for 2 h at room temperature, then concentrated. The residue was purified by silica gel column chromatography, eluting with PE/THF (1:1) to afford 5-cyano-N-[3-[1-(4,5-dimethyl-1-[[2-(trimethylsilyl)ethoxy]methyl]imidazol-2-yl)imidazo[1,5-a]pyridin-6-yl]-2,4-difluorophenyl]-2-methoxypyridine-3-sulfonamide (190 mg, 55% yield) as a brown solid.

LCMS (ES, m/z): [M+H]⁺: 666.

### Synthesis of Compound 126: 5-cyano-N-[3-[1-(4,5-dimethyl-1H-imidazol-2-yl)imidazo[1,5-a]pyridin-6-yl]-2,4-difluorophenyl]-2-methoxypyridine-3-sulfonamide

Into an 8 mL vial were added 5-cyano-N-[3-[1-(4,5-dimethyl-1-[[2-(trimethylsilyl)ethoxy]methyl]imidazol-2-yl) imidazo[1,5-a]pyridin-6-yl]-2,4-difluorophenyl]-2-methoxypyridine-3-sulfonamide (180 mg, 0.27 mmol, 1 equiv) and TFA (3 mL). The resulting mixture was stirred for 30 min at 40° C., then concentrated under vacuum. The crude product was purified by Prep-HPLC with the following conditions: Column: XBridge Prep C18 OBD, 5 μm, 19*150 mm; Mobile Phase: 3-33% MeCN/0.05% aqueous ammonia; Flow rate: 20 mL/min; to give 5-cyano-N-[3-[1-(4,5-dimethyl-1H-imidazol-2-yl)imidazo[1,5-a]pyridin-6-yl]-2,4-difluorophenyl]-2-methoxypyridine-3-sulfonamide (31 mg, 22% yield) as a yellow solid.

LCMS (ES, m/z): [M+H]⁺: 536.

¹H NMR (300 MHz, DMSO-d₆) δ 11.58-11.52 (m, 2H), 8.90 (d, J=2.2 Hz, 1H), 8.52-8.43 (m, 3H), 8.19 (d, J=9.4 Hz, 1H), 7.35 (td, J=8.9, 5.8 Hz, 1H), 7.24-7.12 (m, 1H), 6.87-6.77 (m, 1H), 4.01 (s, 3H), 2.14 (s, 6H).

### Example 142: Synthesis of N-[2,4-difluoro-3-[1-(5-methyl-4H-1,2,4-triazol-3-yl)imidazo[1,5-a]pyrazin-6-yl]phenyl]-5-fluoro-2-methoxypyridine-3-sulfonamide (Compound 127)

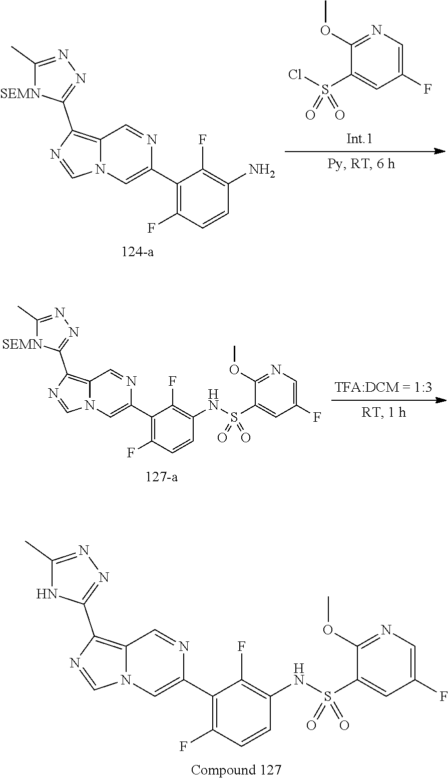 [SMILES: Cc1nnc(-c2ncn3cc(-c4c(F)ccc(N)c4F)ncc23)n1COCC[Si](C)(C)C ; COc1ncc(F)cc1S(=O)(=O)Cl ; COc1ncc(F)cc1S(=O)(=O)Nc1ccc(F)c(-c2cn3cnc(-c4nnc(C)n4COCC[Si](C)(C)C)c3cn2)c1F ; COc1ncc(F)cc1S(=O)(=O)Nc1ccc(F)c(-c2cn3cnc(-c4nnc(C)[nH]4)c3cn2)c1F]

### Synthesis of 127-a: N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-5-fluoro-2-methoxypyridine-3-sulfonamide

To a stirred solution of 2,4-difluoro-3-[1-(5-methyl-4-[[2-(trimethylsilyl)ethoxy]methyl]-1,2,4-triazol-3-yl)imidazo[1,5-a]pyrazin-6-yl]aniline (65 mg, 0.14 mmol, 1 equiv) in pyridine (2 mL) was added 5-fluoro-2-methoxypyridine-3-sulfonyl chloride in portions. The mixture was stirred for 3 h then concentrated under reduced pressure. The residue was purified by prep-HPLC Flash-Prep-HPLC with the following conditions: Column, WelFlash™ C18-I, Spherical C18 20-40 μm, 120 g; mobile phase: 35-95% MeCN/0.1% aqueous formic acid; Detector, 220 nm; to give N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-5-fluoro-2-methoxypyridine-3-sulfonamide (60 mg, 65% yield) as a white solid.

### Synthesis of Compounds 127: N-[2,4-difluoro-3-[1-(5-methyl-4H-1,2,4-triazol-3-yl)imidazo[1,5-a]pyrazin-6-yl]phenyl]-5-fluoro-2-methoxypyridine-3-sulfonamide

To a stirred solution of N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-5-fluoro-2-methoxypyridine-3-sulfonamide (55 mg, 0.08 mmol, 1 equiv) in DCM (3 mL) was added TFA (1 mL) dropwise at 0° C. The resulting mixture was stirred for 3 h at room temperature then concentrated under reduced pressure. The residue was purified by prep-HPLC with the following conditions: Column, welch Vltimate XB-C18, 50×250 mm, 10 μm; Mobile Phase: 5-40% MeCN/0.1% aqueous formic acid; Detector, 220 nm; to give N-[2,4-difluoro-3-[1-(5-methyl-4H-1,2,4-triazol-3-yl)imidazo[1,5-a]pyrazin-6-yl]phenyl]-5-fluoro-2-methoxypyridine-3-sulfonamide (35 mg, 80% yield) as a white solid.

LCMS (ES, m/z): [M+H]⁺: 517

¹H NMR (300 MHz, DMSO-d₆) δ 14.03 (d, J=144.2 Hz, 1H), 10.43 (s, 1H), 9.55 (d, J=1.8 Hz, 1H), 8.61 (d, J=17.6 Hz, 1H), 8.46 (d, J=3.0 Hz, 1H), 8.03 (dd, J=7.3, 3.0 Hz, 1H), 7.41 (s, 1H), 7.23 (t, J=9.2 Hz, 1H), 3.92 (s, 3H), 2.41 (d, J=30.1 Hz, 3H).

### Example 143: Synthesis of 5-cyano-N-[2,4-difluoro-3-[1-(5-methyl-4H-1,2,4-triazol-3-yl)imidazo[1,5-a]pyrazin-6-yl]phenyl]-2-methoxypyridine-3-sulfonamide (Compound 128)

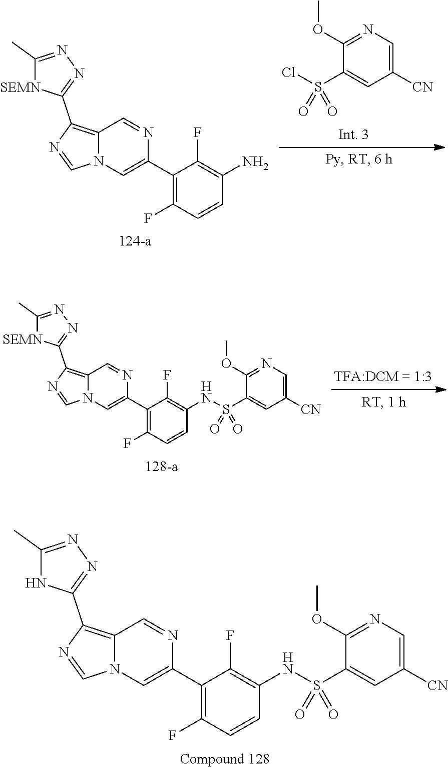 [SMILES: Cc1nnc(-c2ncn3cc(-c4c(F)ccc(N)c4F)ncc23)n1COCC[Si](C)(C)C ; COc1ncc(C#N)cc1S(=O)(=O)Cl ; COc1ncc(C#N)cc1S(=O)(=O)Nc1ccc(F)c(-c2cn3cnc(-c4nnc(C)n4COCC[Si](C)(C)C)c3cn2)c1F ; COc1ncc(C#N)cc1S(=O)(=O)Nc1ccc(F)c(-c2cn3cnc(-c4nnc(C)[nH]4)c3cn2)c1F]

### Synthesis of 128-a: 5-cyano-N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-2-methoxypyridine-3-sulfonamide

To a stirred solution of 2,4-difluoro-3-[1-(5-methyl-4-[[2-(trimethylsilyl)ethoxy]methyl]-1,2,4-triazol-3-yl)imidazo[1,5-a]pyrazin-6-yl]aniline (65 mg, 0.14 mmol, 1 equiv) in pyridine (2 mL) was added 5-cyano-2-methoxypyridine-3-sulfonyl chloride (66 mg, 0.28 mmol, 2 equiv) in portions and the mixture stirred for 6 h. The resulting mixture was concentrated and the residue was purified by prep-HPLC with the following conditions: Column, WelFlash™ C18-I, Spherical C18 20-40 μm, 120 g; mobile phase: 25-95% MeCN/0.1% aqueous formic acid; Detector, 220 nm; to give 5-cyano-N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-2-methoxypyridine-3-sulfonamide (65 mg, 70% yield) as a white solid.

LCMS (ES, m/z): [M+H]⁺: 654

### Synthesis of Compound 128: 5-cyano-N-[2,4-difluoro-3-[1-(5-methyl-4H-1,2,4-triazol-3-yl)imidazo[1,5-a]pyrazin-6-yl]phenyl]-2-methoxypyridine-3-sulfonamide

To a stirred solution of 5-cyano-N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-2-methoxypyridine-3-sulfonamide (60 mg) in DCM (3 mL) was added TFA (1 mL) dropwise at 0° C. The resulting mixture was stirred for 1 h then concentrated under reduced pressure. The residue was purified by prep-HPLC with the following conditions: Column, welch Vltimate XB-C18, 50×250 mm, 10 μm; Mobile Phase: 3-29% MeCN/0.05% aqueous ammonia; Detector, 220 nm; to give 5-cyano-N-[2,4-difluoro-3-[1-(5-methyl-4H-1,2,4-triazol-3-yl)imidazo[1,5-a]pyrazin-6-yl]phenyl]-2-methoxypyridine-3-sulfonamide (25 mg) as a white solid.

LCMS (ES, m/z): [M+H]⁺: 524

1H NMR (300 MHz, DMSO-d6) δ 14.04 (br s, 1H), 10.56 (br s, 1H), 9.55 (br s, 1H), 8.91 (br s, 1H), 8.90-8.40 (m, 3H), 7.42 (br s, 1H), 7.21 (br s, 1H), 4.01 (s, 3H), 2.39 (s, 3H).

### Example 144: Synthesis of N-[2,4-difluoro-3-[1-(5-methyl-4H-1,2,4-triazol-3-yl)imidazo[1,5-a]pyrazin-6-yl]phenyl]-5-fluoro-2-methylpyridine-3-sulfonamide (Compound 129)

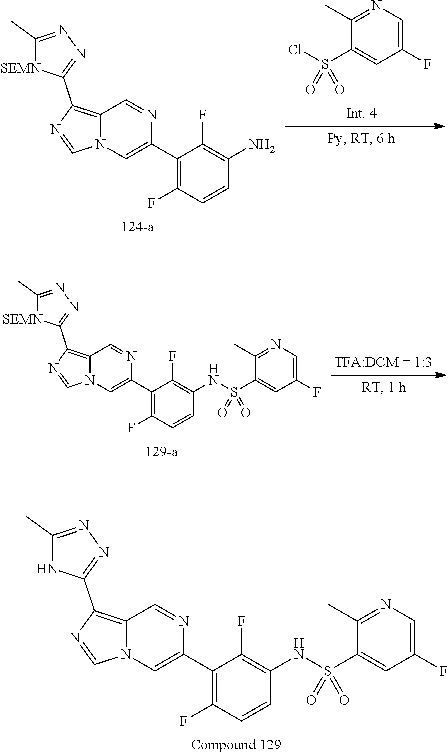 [SMILES: Cc1nnc(-c2ncn3cc(-c4c(F)ccc(N)c4F)ncc23)n1COCC[Si](C)(C)C ; Cc1ncc(F)cc1S(=O)(=O)Cl ; Cc1ncc(F)cc1S(=O)(=O)Nc1ccc(F)c(-c2cn3cnc(-c4nnc(C)n4COCC[Si](C)(C)C)c3cn2)c1F ; Cc1nnc(-c2ncn3cc(-c4c(F)ccc(NS(=O)(=O)c5cc(F)cnc5C)c4F)ncc23)[nH]1]

### Synthesis of 129-a: N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-5-fluoro-2-methylpyridine-3-sulfonamide

To a stirred solution of 2,4-difluoro-3-[1-(5-methyl-4-[[2-(trimethylsilyl)ethoxy]methyl]-1,2,4-triazol-3-yl)imidazo[1,5-a]pyrazin-6-yl]aniline (100 mg, 0.22 mmol, 1 equiv) in pyridine (2 mL) was added 5-fluoro-2-methylpyridine-3-sulfonyl chloride (137 mg, 0.66 mmol, 3 equiv) in portions at room temperature. The resulting mixture was stirred for 6 h then concentrated under reduced pressure. The residue was purified by prep-HPLC with the following conditions: Column, WelFlash™ C18-I, Spherical C18 20-40 μm, 120 g; mobile phase: 25-95% MeCN/0.1% aqueous formic acid; Detector, 220 nm; to give N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-5-fluoro-2-methylpyridine-3-sulfonamide (100 mg, 73% yield) as a white solid.

LCMS (ES, m/z): [M+H]⁺: 631

### Synthesis of Compound 129: N-[2,4-difluoro-3-[1-(5-methyl-4H-1,2,4-triazol-3-yl)imidazo[1,5-a]pyrazin-6-yl]phenyl]-5-fluoro-2-methylpyridine-3-sulfonamide

To a stirred solution of N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-5-fluoro-2-methylpyridine-3-sulfonamide (95 mg, 0.15 mmol, 1 equiv) in DCM (3 mL) in an ice bath was added TFA (1 mL) dropwise. The resulting mixture was stirred for 1 h at room temperature then concentrated under reduced pressure. The residue was purified by prep-HPLC with the following conditions: Column, welch Vltimate XB-C18, 50×250 mm, 10 μm; Mobile Phase: 10-45% MeCN/0.1% aqueous formic acid; Detector, 220 nm; to give N-[2,4-difluoro-3-[1-(5-methyl-4H-1,2,4-triazol-3-yl)imidazo[1,5-a]pyrazin-6-yl]phenyl]-5-fluoro-2-methylpyridine-3-sulfonamide (44 mg, 58% yield) as a white solid.

LCMS (ES, m/z): [M+H]⁺: 501

¹H NMR (300 MHz, DMSO-d₆) δ 14.02 (d, J=143.3 Hz, 1H), 10.79 (s, 1H), 9.54 (d, J=1.6 Hz, 1H), 8.78-8.68 (m, 1H), 8.64 (s, 1H), 8.56 (s, 1H), 7.95 (dd, J=8.2, 2.8 Hz, 1H), 7.39 (q, J=8.5 Hz, 1H), 7.28-7.16 (m, 1H), 2.78 (d, J=1.2 Hz, 3H), 2.41 (d, J=29.6 Hz, 3H).

### Example 145: Synthesis of 5-chloro-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methoxypyridine-3-sulfonamide & 5-chloro-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methoxypyridine-3-sulfonamide (Compounds 130-1 & 130-2)

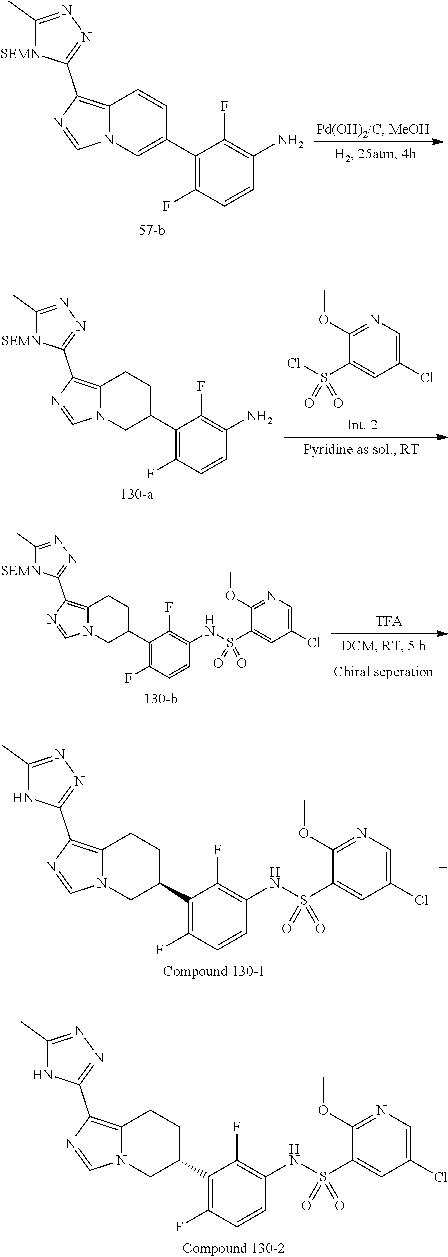

### Synthesis of 130-a: 2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]aniline

Into a 50 mL pressure tank reactor was placed 2,4-difluoro-3-[1-(5-methyl-4-[[2-(trimethylsilyl)ethoxy]methyl]-1,2,4-triazol-3-yl)imidazo[1,5-a]pyridin-6-yl]aniline (500 mg, 1.1 mmol, 1 equiv) and MeOH (20 mL). 20% Pd(OH)₂/C (500 mg) was added and the mixture stirred under 25 atm of hydrogen gas for 4 h at 70° C. The reaction mixture was cooled and filtered. The filtrate was concentrated to give 2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]aniline (600 mg, 92% yield) as an off-white solid.

LCMS (ES, m/z): [M+H]⁺: 461

### Synthesis of 130-b: 5-chloro-N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-2-methoxypyridine-3-sulfonamide

Into a 40 mL vial was placed 2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]aniline (120 mg, 0.26 mmol, 1 equiv), pyridine (4 mL) and 5-chloro-2-methoxypyridine-3-sulfonyl chloride (95 mg, 0.39 mmol, 1.5 equiv). The resulting solution was stirred overnight then concentrated. The crude product was purified by Flash-Prep-HPLC with the following conditions: Column, WelFlash™ C18-I, Spherical C18 20-40 m, 120 g; mobile phase: 10-90% MeCN/0.1% aqueous formic acid; Detector, 220 nm; to give 5-chloro-N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-2-methoxypyridine-3-sulfonamide (100 mg, 58% yield) as a white solid.

LCMS (ES, m/z): [M+H]⁺: 666

### Synthesis of Compounds 130-1 & 130-2: 5-chloro-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methoxypyridine-3-sulfonamide & 5-chloro-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methoxypyridine-3-sulfonamide

Into a 40 mL vial was placed 5-chloro-N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-2-methoxypyridine-3-sulfonamide (100 mg, 0.15 mmol, 1 equiv), DCM (3 mL) and TFA (1 mL). The resulting solution was stirred for 5 h then concentrated. The crude product was purified by Flash-Prep-HPLC with the following conditions: Column, WelFlash™ C18-I, Spherical C18 20-40 m, 120 g; mobile phase: 10-85% MeCN/0.1% aqueous formic acid; Detector, 220 nm to give the racemic product. The enantiomers were separated using Chiral-Prep-HPLC with the following conditions: Column: CHIRAL ART Cellulose-SB, 3*25 cm, 5 μm; Mobile Phase: 25% EtOH/hexane containing 0.1% 2 M NH₃ in MeOH; Flow rate: 55 mL/min; to give the single enantiomers 5-chloro-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methoxypyridine-3-sulfonamide (RT 13.80 mins, 10 mg, 12% yield, stereochemistry randomly assigned) as a white solid and 5-chloro-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methoxypyridine-3-sulfonamide (RT 15.80 mins, 10 mg 12% yield, stereochemistry opposite to randomly assigned isomer) as a white solid.

LCMS (ES, m/z): [M+H]⁺: 536 for both isomers

130-1: ¹ NMR (300 MHz, DMSO-d₆) δ 13.56 (s, 1H), 10.34 (s, 1H), 8.50 (q, J=2.5 Hz, 1H), 8.02 (q, J=2.5 Hz, 1H), 7.65 (s, 1H), 7.25 (s, 1H), 7.10 (t, J=9.6 Hz, 1H), 4.35-4.23 (m, 1H), 4.07-3.93 (m, 1H), 3.90 (s, 3H), 3.61-3.43 (m, 1H), 3.41-3.32 (m, 1H), 3.01-2.79 (m, 1H), 2.26 (s, 3H), 2.17-1.89 (m, 2H). 130-2: ¹H NMR (300 MHz, DMSO-d6) δ 13.56 (s, 1H), 10.37 (s, 1H), 8.49 (d, J=2.6 Hz, 1H), 8.01 (d, J=2.5 Hz, 1H), 7.65 (s, 1H), 7.38-7.18 (m, 1H), 7.15-7.02 (m, 1H), 4.34-4.24 (m, 1H), 4.09-3.96 (m, 1H), 3.92 (s, 3H), 3.59-3.43 (m, 1H), 3.43-3.34 (m, 1H), 3.00-2.92 (m, 1H), 2.26 (s, 3H), 2.17-1.91 (m, 2H).

### Example 146: Synthesis of N-[2,4-difluoro-3-[(6S)-1-(5-methyl-4H-1,2,4-triazol-3-yl)-5H,6H,7H,8H-imidazo[1,5-a]pyridin-6-yl]phenyl]-5-fluoro-2-methoxypyridine-3-sulfonamide & N-[2,4-difluoro-3-[(6R)-1-(5-methyl-4H-1,2,4-triazol-3-yl)-5H,6H,7H,8H-imidazo[1,5-a]pyridin-6-yl]phenyl]-5-fluoro-2-methoxypyridine-3-sulfonamide (Compounds 131-1 & 131-2)

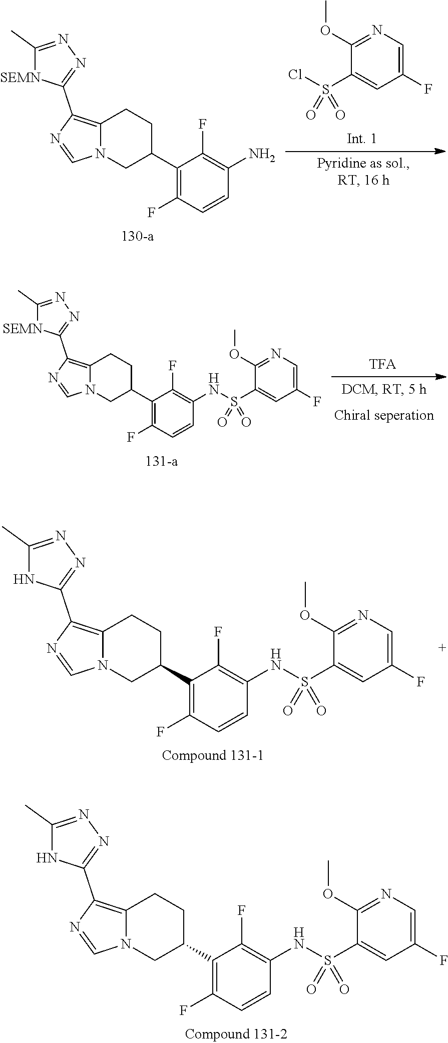 [SMILES: Cc1nnc(-c2ncn3c2CCC(c2c(F)ccc(N)c2F)C3)n1COCC[Si](C)(C)C ; COc1ncc(F)cc1S(=O)(=O)Cl ; COc1ncc(F)cc1S(=O)(=O)Nc1ccc(F)c(C2CCc3c(-c4nnc(C)n4COCC[Si](C)(C)C)ncn3C2)c1F ; COc1ncc(F)cc1S(=O)(=O)Nc1ccc(F)c([C@@H]2CCc3c(-c4nnc(C)[nH]4)ncn3C2)c1F ; COc1ncc(F)cc1S(=O)(=O)Nc1ccc(F)c([C@H]2CCc3c(-c4nnc(C)[nH]4)ncn3C2)c1F]

### Synthesis of 131-a: N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-5-fluoro-2-methoxypyridine-3-sulfonamide

Into a 40 mL vial was placed 2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]aniline (120 mg, 0.26 mmol, 1 equiv), pyridine (4 mL) and 5-fluoro-2-methoxypyridine-3-sulfonyl chloride (88 mg, 0.39 mmol, 1.5 equiv). The resulting solution was stirred overnight then concentrated. The crude product was purified by Flash-Prep-HPLC with the following conditions: Column, welch Vltimate XB-C18, 50×250 mm, 10 μm; Mobile Phase: 20-90% MeCN/0.1% aqueous formic acid to give N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-5-fluoro-2-methoxypyridine-3-sulfonamide (93 mg, 55% yield) as a white solid.

LCMS (ES, m/z): [M+H]⁺: 650

### Synthesis of Compounds 131-1 & 131-2: N-[2,4-difluoro-3-[(6 S)-1-(5-methyl-4H-1,2,4-triazol-3-yl)-5H,6H,7H,8H-imidazo[1,5-a]pyridin-6-yl]phenyl]-5-fluoro-2-methoxypyridine-3-sulfonamide & N-[2,4-difluoro-3-[(6R)-1-(5-methyl-4H-1,2,4-triazol-3-yl)-5H,6H,7H,8H-imidazo[1,5-a]pyridin-6-yl]phenyl]-5-fluoro-2-methoxypyridine-3-sulfonamide

Into a 40 mL vial was placed N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-5-fluoro-2-methoxypyridine-3-sulfonamide (90 mg, 0.14 mmol, 1 equiv), DCM (3 mL) and TFA (1 mL). The resulting solution was stirred for 5 h then concentrated. The crude product was purified by Flash-Prep-HPLC with the following conditions: Column, welch Vltimate XB-C18, 50×250 mm, 10 μm; Mobile Phase: 15-80% MeCN/0.1% aqueous formic acid to give the racemic product. The enantiomers were separated using CHIRAL-Prep-HPLC with the following conditions: Column: CHIRAL ART Cellulose-SB, 3*25 cm, 5 μm; Mobile Phase: 25% EtOH/Hexane containing 0.1% 2 M NH₃ in MeOH; Flow rate: 55 mL/min; to give N-[2,4-difluoro-3-[(6S)-1-(5-methyl-4H-1,2,4-triazol-3-yl)-5H,6H,7H,8H-imidazo[1,5-a]pyridin-6-yl]phenyl]-5-fluoro-2-methoxypyridine-3-sulfonamide (RT 13 mins, 25 mg, 35% yield, stereochemistry randomly assigned) as a white solid and N-[2,4-difluoro-3-[(6R)-1-(5-methyl-4H-1,2,4-triazol-3-yl)-5H,6H,7H,8H-imidazo[1,5-a]pyridin-6-yl]phenyl]-5-fluoro-2-methoxypyridine-3-sulfonamide (RT 16 mins, 10 mg 14% yield, stereochemistry opposite of randomly assigned isomer) as a white solid.

LCMS (ES, m/z): [M+H]⁺: 520 for both isomers

131-1: ¹H NMR (300 MHz, DMSO-d₆) δ 13.56 (br s, 1H), 10.33 (br s, 1H), 8.47 (d, J=3.0 Hz, 1H), 7.97 (dd, J=7.3, 3.0 Hz, 1H), 7.66 (br s, 1H), 7.25 (td, J=8.9, 5.7 Hz, 1H), 7.15-7.05 (m, 1H), 4.30 (dd, J=12.3, 5.2 Hz, 1H), 4.00 (t, J=11.9 Hz, 1H), 3.92 (s, 3H), 3.60-3.45 (m, 1H), 3.44-3.30 (m, 1H), 2.97-2.83 (m, 1H), 2.25 (br s, 3H), 2.30-1.92 (m, 2H). 131-2: ¹H NMR (300 MHz, DMSO-d₆) δ 13.55 (br s, 1H), 8.47 (d, J=3.0 Hz, 1H), 7.98 (dd, J=7.3, 3.0 Hz, 1H), 7.66 (br s, 1H), 7.24 (td, J=8.9, 5.7 Hz, 1H), 7.15-7.05 (m, 1H), 4.30 (dd, J=12.3, 5.2 Hz, 1H), 4.00 (t, J=11.9 Hz, 1H), 3.91 (s, 3H), 3.62-3.47 (m, 1H), 3.40-3.30 (m, 1H), 2.95-2.80 (m, 1H), 2.25 (br s, 3H), 2.30-1.92 (m, 2H).

### Example 147: Synthesis of 6-(3-((5-chloro-2-oxo-1,2-dihydropyridine)-3-sulfonamido)-2,6-difluorophenyl)-N-methylimidazo[1,5-a]pyrazine-1-carboxamide (Compound 132)

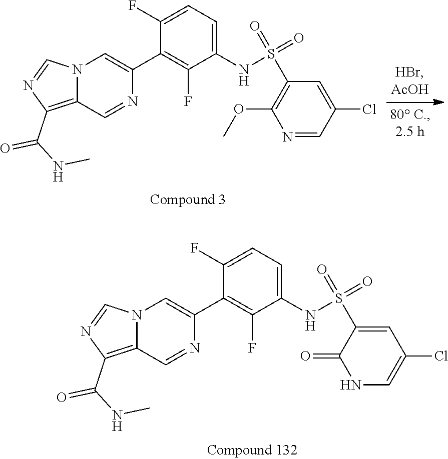 [SMILES: CNC(=O)c1ncn2cc(-c3c(F)ccc(NS(=O)(=O)c4cc(Cl)cnc4OC)c3F)ncc12 ; CNC(=O)c1ncn2cc(-c3c(F)ccc(NS(=O)(=O)c4cc(Cl)c[nH]c4=O)c3F)ncc12]

### Synthesis of Compound 132: 6-(3-((5-chloro-2-oxo-1,2-dihydropyridine)-3-sulfonamido)-2,6-difluorophenyl)-N-methylimidazo[1,5-a]pyrazine-1-carboxamide

HBr (50% aqueous, 48 μL, 0.29 mmol, 3 equiv) was added to a suspension of 6-(3-((5-chloro-2-methoxypyridine)-3-sulfonamido)-2,6-difluorophenyl)-N-methylimidazo[1,5-a]pyrazine-1-carboxamide (50 mg, 0.098 mmol, 1 equiv) in AcOH (1.0 mL) and the mixture was heated to 80° C. for 2.5 hours and then concentrated under reduced pressure. The crude residue was purified by Prep-HPLC with the following conditions: Column, RediSep Prep C18, 100 Å, 5 μm, 150×20 mm; mobile phase: 5-40% MeCN/0.1% aqueous ammonium hydroxide; Detector, 254 nm; to afford 6-(3-((5-chloro-2-oxo-1,2-dihydropyridine)-3-sulfonamido)-2,6-difluorophenyl)-N-methylimidazo[1,5-a]pyrazine-1-carboxamide (15 mg, 31% yield) as a white solid.

LCMS (ES, m/z): [M+H]⁺: 495.

¹H NMR (400 MHz, DMSO-d₆) δ 12.83 (br s, 1H), 9.52 (dd, J=1.7, 0.6 Hz, 1H), 8.71-8.68 (m, 1H), 8.66 (d, J=0.6 Hz, 1H), 8.43 (q, J=4.7 Hz, 1H), 8.05 (d, J=2.9 Hz, 1H), 7.41 (td, J=8.9, 5.8 Hz, 1H), 7.23 (td, J=9.1, 1.6 Hz, 1H), 2.84 (d, J=4.8 Hz, 3H).

### Example 148: Synthesis of 5-chloro-N-[2,4-difluoro-3-[1-(5-methyl-4H-1,2,4-triazol-3-yl)imidazo[1,5-a]pyrazin-6-yl]phenyl]-2-methylpyridine-3-sulfonamide (Compound 133)

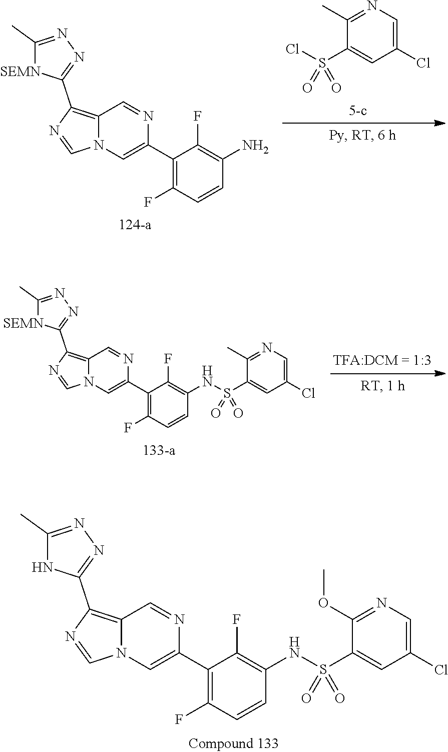 [SMILES: Cc1nnc(-c2ncn3cc(-c4c(F)ccc(N)c4F)ncc23)n1COCC[Si](C)(C)C ; Cc1ncc(Cl)cc1S(=O)(=O)Cl ; Cc1ncc(Cl)cc1S(=O)(=O)Nc1ccc(F)c(-c2cn3cnc(-c4nnc(C)n4COCC[Si](C)(C)C)c3cn2)c1F ; COc1ncc(Cl)cc1S(=O)(=O)Nc1ccc(F)c(-c2cn3cnc(-c4nnc(C)[nH]4)c3cn2)c1F]

### Synthesis of 133-a: 5-chloro-N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-2-methylpyridine-3-sulfonamide

To a stirred solution of 2,4-difluoro-3-[1-(5-methyl-4-[[2-(trimethylsilyl)ethoxy]methyl]-1,2,4-triazol-3-yl)imidazo[1,5-a]pyrazin-6-yl]aniline (100 mg, 0.22 mmol, 1 equiv) in pyridine (2 mL) was added 5-chloro-2-methylpyridine-3-sulfonyl chloride (74 mg, 0.33 mmol, 1.5 equiv) in portions at room temperature. The resulting mixture was stirred for 6 h then concentrated under reduced pressure. The residue was purified by prep-HPLC Flash-Prep-HPLC with the following conditions: Column, WelFlash™ C18-I, Spherical C18 20-40 μm, 120 g; mobile phase: 25-95% MeCN/0.1% aqueous formic acid; to give 5-chloro-N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-2-methylpyridine-3-sulfonamide (70 mg, 49% yield) as a white solid.

LCMS (ES, m/z): [M+H]⁺: 647

### Synthesis of Compound 133: 5-chloro-N-[2,4-difluoro-3-[1-(5-methyl-4H-1,2,4-triazol-3-yl)imidazo[1,5-a]pyrazin-6-yl]phenyl]-2-methylpyridine-3-sulfonamide

To a stirred solution of 5-chloro-N-[2,4-difluoro-3-[1-(5-methyl-4-[[2-(trimethylsilyl)ethoxy]methyl]-1,2,4-triazol-3-yl)imidazo[1,5-a]pyrazin-6-yl]phenyl]-2-methylpyridine-3-sulfonamide (65 mg) in DCM (3 mL) in an ice bath was added TFA (1 mL) dropwise. The resulting mixture was stirred for 1 h at room temperature then concentrated under reduced pressure. The residue was purified by prep-HPLC with the following conditions: Column, welch Vltimate XB-C18, 50×250 mm, 10 μm; Mobile Phase: 10-50% MeCN/0.1% aqueous formic acid; to give 5-chloro-N-[2,4-difluoro-3-[1-(5-methyl-4H-1,2,4-triazol-3-yl)imidazo[1,5-a]pyrazin-6-yl]phenyl]-2-methylpyridine-3-sulfonamide (32 mg) as a white solid.

LCMS (ES, m/z): [M+H]⁺: 517

¹H NMR (300 MHz, DMSO-d₆) δ 14.03 (d, J=143.1 Hz, 1H), 10.79 (s, 1H), 9.54 (d, J=1.6 Hz, 1H), 8.76 (d, J=2.4 Hz, 1H), 8.65 (s, 1H), 8.61-8.54 (m, 1H), 8.09 (d, J=2.4 Hz, 1H), 7.40 (td, J=8.8, 5.7 Hz, 1H), 7.31-7.19 (m, 1H), 2.78 (s, 3H), 2.42 (d, J=27.7 Hz, 3H).

### Example 149: Synthesis of N-[2,4-difluoro-3-[(6S)-1-(5-methyl-4H-1,2,4-triazol-3-yl)-5H,6H,7H,8H-imidazo[1,5-a]pyridin-6-yl]phenyl]-5-fluoro-2-methylpyridine-3-sulfonamide & N-[2,4-difluoro-3-[(6R)-1-(5-methyl-4H-1,2,4-triazol-3-yl)-5H,6H,7H,8H-imidazo[1,5-a]pyridin-6-yl]phenyl]-5-fluoro-2-methylpyridine-3-sulfonamide (Compounds 134-1 & 134-2)

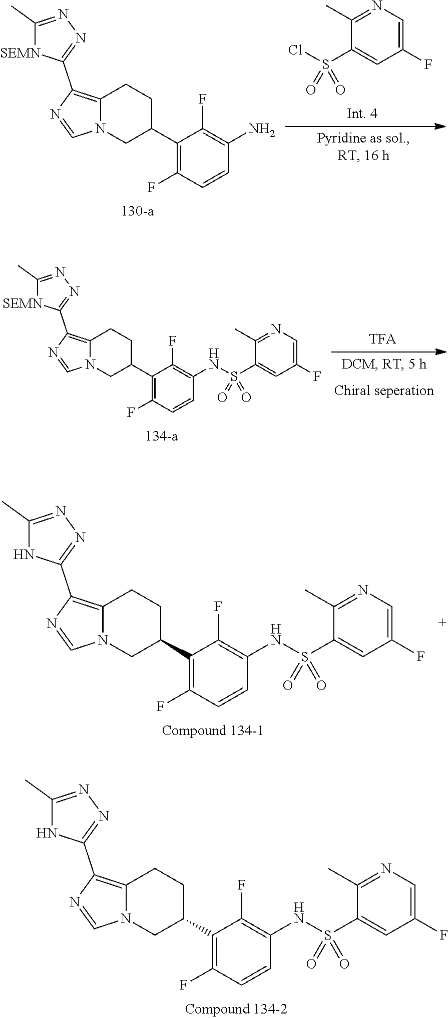 [SMILES: Cc1nnc(-c2ncn3c2CCC(c2c(F)ccc(N)c2F)C3)n1COCC[Si](C)(C)C ; Cc1ncc(F)cc1S(=O)(=O)Cl ; Cc1ncc(F)cc1S(=O)(=O)Nc1ccc(F)c(C2CCc3c(-c4nnc(C)n4COCC[Si](C)(C)C)ncn3C2)c1F ; Cc1nnc(-c2ncn3c2CC[C@@H](c2c(F)ccc(NS(=O)(=O)c4cc(F)cnc4C)c2F)C3)[nH]1 ; Cc1nnc(-c2ncn3c2CC[C@H](c2c(F)ccc(NS(=O)(=O)c4cc(F)cnc4C)c2F)C3)[nH]1]

### Synthesis of 134-a: N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-5-fluoro-2-methylpyridine-3-sulfonamide

Into a 40 mL vial was placed 2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]aniline (120 mg, 0.26 mmol, 1 equiv), pyridine (4 mL) and 5-fluoro-2-methylpyridine-3-sulfonyl chloride (82 mg, 0.39 mmol, 1.5 equiv). The resulting solution was stirred overnight then concentrated. The crude product was purified by Flash-Prep-HPLC with the following conditions: Column, WelFlash™ C18-I, Spherical C18 20-40 m, 120 g; mobile phase: 30-85% MeCN/0.1% aqueous formic acid; to give N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-5-fluoro-2-methylpyridine-3-sulfonamide (88 mg, 53% yield) as a white solid.

LCMS (ES, m/z): [M+H]⁺: 634

### Synthesis of Compounds 134-1 & 134-2: N-[2,4-difluoro-3-[(6S)-1-(5-methyl-4H-1,2,4-triazol-3-yl)-5H,6H,7H,8H-imidazo[1,5-a]pyridin-6-yl]phenyl]-5-fluoro-2-methylpyridine-3-sulfonamide & N-[2,4-difluoro-3-[(6R)-1-(5-methyl-4H-1,2,4-triazol-3-yl)-5H,6H,7H,8H-imidazo[1,5-a]pyridin-6-yl]phenyl]-5-fluoro-2-methylpyridine-3-sulfonamide

Into a 40 mL vial was placed N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-5-fluoro-2-methoxypyridine-3-sulfonamide (90 mg, 0.14 mmol, 1 equiv), DCM (3 mL) and TFA (1 mL). The resulting solution was stirred for 5 h then concentrated under vacuum. The residue was purified by prep-HPLC with the following conditions: Column, welch Vltimate XB-C18, 50×250 mm, 10 μm; Mobile Phase: 15-75% MeCN/0.1% aqueous formic acid to give the racemic product. The enantiomers were separated using Chiral-Prep-HPLC; Column: CHIRALPAK IE, 3*25 cm, 5 μm; Mobile Phase: 18% MeOH/MTBE containing 0.2% diethylamine; Flow rate: 35 mL/min; to give N-[2,4-difluoro-3-[(6S)-1-(5-methyl-4H-1,2,4-triazol-3-yl)-5H,6H,7H,8H-imidazo[1,5-a]pyridin-6-yl]phenyl]-5-fluoro-2-methylpyridine-3-sulfonamide (RT 17 mins, 25 mg, 35% yield, stereochemistry randomly assigned) as a white solid, and N-[2,4-difluoro-3-[(6R)-1-(5-methyl-4H-1,2,4-triazol-3-yl)-5H,6H,7H,8H-imidazo[1,5-a]pyridin-6-yl]phenyl]-5-fluoro-2-methylpyridine-3-sulfonamide (RT 19 mins, 10 mg 14% yield, stereochemistry assigned as opposite to other enantiomer) as a white solid.

LCMS (ES, m/z): [M+H]^{m}: 504 for both isomers

134-1: ¹H NMR (300 MHz, DMSO-d₆) δ 13.56 (br s, 1H), 10.66 (br s, 1H), 8.70 (d, J=2.8 Hz, 1H), 7.87 (dd, J=8.3, 2.9 Hz, 1H), 7.65 (s, 1H), 7.22 (td, J=8.9, 5.7 Hz, 1H), 7.07 (t, J=9.4 Hz, 1H), 4.27 (dd, J=12.3, 5.2 Hz, 1H), 3.97 (t, J=12.0 Hz, 1H), 3.61-3.43 (m, 1H), 3.43-3.33 (m, 1H), 2.93-2.87 (m, 1H), 2.74 (d, J=1.2 Hz, 3H), 2.26 (s, 3H), 2.17-1.90 (m, 2H).

134-2: ¹H NMR (300 MHz, DMSO-d₆) δ 13.56 (br s, 1H), 10.69 (br s, 1H), 8.74 (d, J=2.8 Hz, 1H), 7.87 (dd, J=8.2, 2.8 Hz, 1H), 7.64 (s, 1H), 7.25 (td, J=8.8, 5.7 Hz, 1H), 7.17-7.05 (m, 1H), 4.27 (dd, J=12.2, 5.1 Hz, 1H), 3.96 (t, J=11.9 Hz, 1H), 3.53-3.43 (m, 1H), 3.42-3.33 (m, 1H), 2.89 (ddd, J=17.6, 11.6, 6.4 Hz, 1H), 2.74 (d, J=1.2 Hz, 3H), 2.27 (s, 3H), 2.17-1.87 (m, 2H).

### Example 150: Synthesis of 5-cyano-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methoxypyridine-3-sulfonamide & 5-cyano-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methoxypyridine-3-sulfonamide (Compounds 135-1 & 135-2)

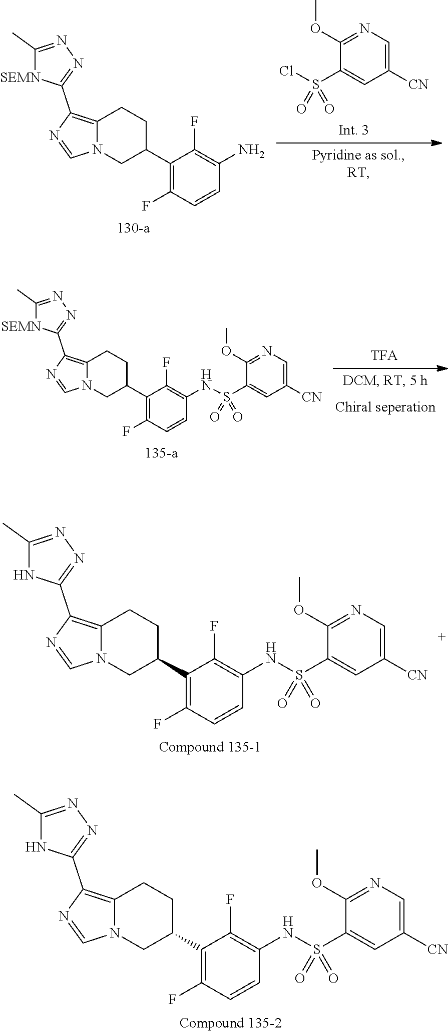 [SMILES: Cc1nnc(-c2ncn3c2CCC(c2c(F)ccc(N)c2F)C3)n1COCC[Si](C)(C)C ; COc1ncc(C#N)cc1S(=O)(=O)Cl ; COc1ncc(C#N)cc1S(=O)(=O)Nc1ccc(F)c(C2CCc3c(-c4nnc(C)n4COCC[Si](C)(C)C)ncn3C2)c1F ; COc1ncc(C#N)cc1S(=O)(=O)Nc1ccc(F)c([C@@H]2CCc3c(-c4nnc(C)[nH]4)ncn3C2)c1F ; COc1ncc(C#N)cc1S(=O)(=O)Nc1ccc(F)c([C@H]2CCc3c(-c4nnc(C)[nH]4)ncn3C2)c1F]

### Synthesis of 135-a: 5-cyano-N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-2-methoxypyridine-3-sulfonamide

Into a 40 mL vial was placed 2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]aniline (120 mg, 0.26 mmol, 1 equiv), pyridine (4 mL) and 5-cyano-2-methoxypyridine-3-sulfonyl chloride (91 mg, 0.4 mmol, 1.5 equiv). The resulting solution was stirred for 16 h then concentrated. The crude product was purified by Flash-Prep-HPLC with the following conditions: Column, WelFlash™ C18-I, Spherical C18 20-40 m, 120 g; mobile phase: 20-90% MeCN/0.1% aqueous formic acid; to give 5-cyano-N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-2-methoxypyridine-3-sulfonamide (90 mg, 53% yield) as a white solid.

LCMS (ES, m/z): [M+H]⁺: 657

### Synthesis of Compounds 135-1 & 135-2: 5-cyano-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methoxypyridine-3-sulfonamide & 5-cyano-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methoxypyridine-3-sulfonamide

Into an 8 mL vial was placed 5-cyano-N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-2-methoxypyridine-3-sulfonamide (90 mg, 0.14 mmol, 1 equiv), DCM (3 mL) and TFA (1 mL). The resulting solution was stirred for 5 h then concentrated under vacuum. The crude product was purified by Flash-Prep-HPLC with the following conditions: Column, WelFlash™ C18-I, Spherical C18 20-40 m, 120 g; mobile phase: 20-90% MeCN/0.1% aqueous formic acid; to give the racemic product. The enantiomers were separated by Chiral-Prep-HPLC with the following conditions: Column: CHIRALPAK IE, 2*25 cm, 5 μm; Mobile Phase: 18% MeOH/MTBE (containing 0.1% DEA; Flow rate: 35 mL/min; to give 5-cyano-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methoxypyridine-3-sulfonamide (RT 19 mins, 10 mg, 14% yield, stereochemistry randomly assigned) and 5-cyano-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methoxypyridine-3-sulfonamide (RT 21 mins, 15 mg, 21% yield, stereochemistry assigned as opposite to other enantiomer) as white solids.

LCMS (ES, m/z): [M+H]⁺: 527 for both isomers

135-1: ¹H NMR (300 MHz, DMSO-d₆) δ 13.56 (br s, 1H), 8.83 (d, J=2.3 Hz, 1H), 8.36 (d, J=2.3 Hz, 1H), 7.66 (s, 1H), 7.23-7.12 (m, 1H), 6.96 (t, J=9.5 Hz, 1H), 4.35-4.25 (m, 1H), 4.05 (d, J=11.4 Hz, 1H), 3.97 (s, 3H), 3.60-3.37 (m, 2H), 3.00-2.83 (m, 1H), 2.26 (s, 3H), 2.18-1.96 (m, 2H).

135-2: ¹H NMR (300 MHz, DMSO-d₆) δ 13.57 (br s, 1H), 10.44 (br s, 1H), 8.91 (s, 1H), 8.42 (d, J=2.2 Hz, 1H), 7.66 (s, 1H), 7.28-7.21 (m, 1H), 7.07 (t, J=9.5 Hz, 1H), 4.29 (s, 1H), 4.09-3.91 (m, 4H), 3.62-3.44 (m, 1H), 3.43-3.35 (m, 1H), 2.94-2.88 (m, 1H), 2.25 (s, 3H), 2.18-1.98 (m, 2H).

### Example 151: Synthesis of 5-chloro-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methylpyridine-3-sulfonamide & 5-chloro-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methylpyridine-3-sulfonamide (Compounds 136-1 & 136-2)

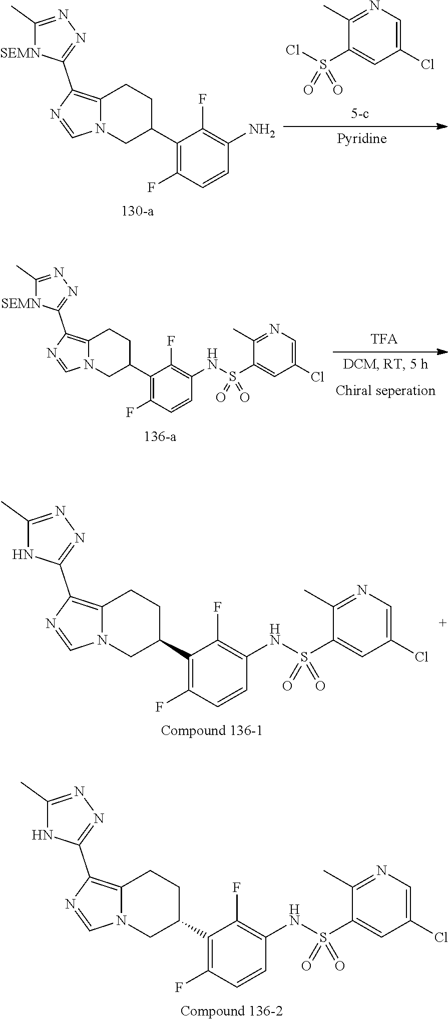 [SMILES: Cc1nnc(-c2ncn3c2CCC(c2c(F)ccc(N)c2F)C3)n1COCC[Si](C)(C)C ; Cc1ncc(Cl)cc1S(=O)(=O)Cl ; Cc1ncc(Cl)cc1S(=O)(=O)Nc1ccc(F)c(C2CCc3c(-c4nnc(C)n4COCC[Si](C)(C)C)ncn3C2)c1F ; Cc1nnc(-c2ncn3c2CC[C@@H](c2c(F)ccc(NS(=O)(=O)c4cc(Cl)cnc4C)c2F)C3)[nH]1 ; Cc1nnc(-c2ncn3c2CC[C@H](c2c(F)ccc(NS(=O)(=O)c4cc(Cl)cnc4C)c2F)C3)[nH]1]

### Synthesis of 136-a: 5-chloro-N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-2-methylpyridine-3-sulfonamide

Into a 40 mL vial was placed 2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]aniline (120 mg, 0.26 mmol, 1 equiv), pyridine (4 mL) and 5-chloro-2-methylpyridine-3-sulfonyl chloride (77 mg, 0.34 mmol, 1.3 equiv). The resulting solution was stirred overnight then concentrated. The crude product was purified by Flash-Prep-HPLC with the following conditions: Column, WelFlash™ C18-I, Spherical C18 20-40 m, 120 g; mobile phase: 20-90% MeCN/0.1% aqueous formic acid; to give 5-chloro-N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-2-methylpyridine-3-sulfonamide (88 mg, 52% yield) as a white solid.

LCMS (ES, m/z): [M+H]⁺: 650

### Synthesis of Compounds 136-1 & 136-2: 5-chloro-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methylpyridine-3-sulfonamide & 5-chloro-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methylpyridine-3-sulfonamide

Into a 40 mL vial was placed 5-chloro-N-[2,4-difluoro-3-[1-(5-methyl-4-[[2-(trimethylsilyl)ethoxy]methyl]-1,2,4-triazol-3-yl)-5H,6H,7H,8H-imidazo[1,5-a]pyridin-6-yl]phenyl]-2-methylpyridine-3-sulfonamide (100 mg, 0.15 mmol, 1 equiv), DCM (3 mL) and TFA (1 mL). The resulting solution was stirred for 5 h then concentrated under vacuum. The crude product was purified by Flash-Prep-HPLC with the following conditions: Column, WelFlash™ C18-I, Spherical C18 20-40 m, 120 g; mobile phase: 20-90% MeCN/0.1% aqueous formic acid to give the racemic product. The enantiomers were separated via Chiral-Prep-HPLC with the following conditions: Column: CHIRALPAK IE, 3*25 cm, 5 μm; Mobile Phase 15% MeOH/MTBE containing 0.1% DEA; Flow rate: 35 mL/min; to give 5-chloro-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methylpyridine-3-sulfonamide (RT 23 mins, 10 mg, 13% yield, stereochemistry randomly assigned) as a white solid and 5-chloro-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methylpyridine-3-sulfonamide (RTRT 27 mins, 10 mg, 13% yield, stereochemistry opposite to randomly assigned isomer) as a white solid.

LCMS (ES, m/z): [M+H]⁺: 520 for both isomers

136-1: ¹H NMR (300 MHz, DMSO-d₆) δ 13.57 (br s, 1H), 10.69 (br s, 1H), 8.77 (d, J=2.4 Hz, 1H), 7.98 (d, J=2.4 Hz, 1H), 7.64 (s, 1H), 7.26 (td, J=8.8, 5.7 Hz, 1H), 7.12 (t, J=9.3 Hz, 1H), 4.27 (dd, J=12.4, 5.1 Hz, 1H), 3.96 (t, J=11.9 Hz, 1H), 3.78 (s, 3H), 3.60-3.43 (m, 1H), 3.43-3.33 (m, 1H), 3.02-2.81 (m, 1H), 2.27 (br s, 3H), 2.19-1.87 (m, 2H).

136-2: ¹H NMR (300 MHz, DMSO-d₆) δ 13.56 (br s, 1H), 10.68 (br s, 1H), 8.77 (d, J=2.4 Hz, 1H), 7.98 (d, J=2.4 Hz, 1H), 7.64 (s, 1H), 7.26 (td, J=8.8, 5.7 Hz, 1H), 7.19-7.04 (m, 1H), 4.27 (dd, J=12.3, 5.2 Hz, 1H), 3.96 (t, J=11.9 Hz, 1H), 3.59-3.41 (m, 1H), 3.43-3.30 (m, 1H), 2.98-2.78 (m, 1H), 2.75 (s, 3H), 2.28 (s, 3H), 2.20-1.87 (m, 2H).

### Example 152: Synthesis of 6-[6-chloro-3-(5-chloro-2-methoxypyridine-3-sulfonamido)-2-fluorophenyl]-N-methylimidazo[1,5-a]pyridine-1-carboxamide (Compound 137)

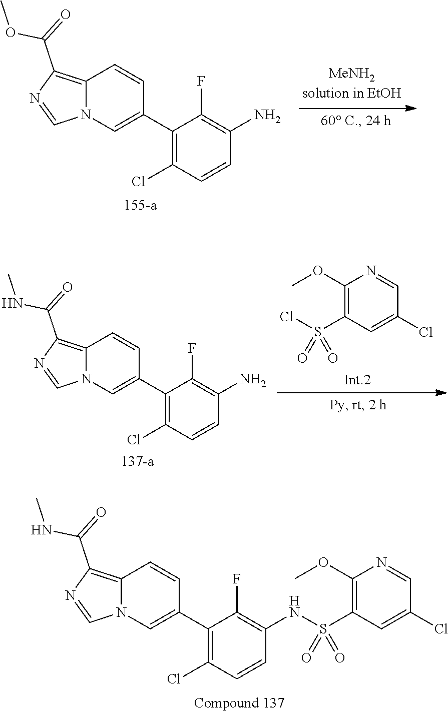 [SMILES: COC(=O)c1ncn2cc(-c3c(Cl)ccc(N)c3F)ccc12 ; CNC(=O)c1ncn2cc(-c3c(Cl)ccc(N)c3F)ccc12 ; COc1ncc(Cl)cc1S(=O)(=O)Cl ; CNC(=O)c1ncn2cc(-c3c(Cl)ccc(NS(=O)(=O)c4cc(Cl)cnc4OC)c3F)ccc12]

### Synthesis of 137-a: 6-(3-amino-6-chloro-2-fluorophenyl)-N-methylimidazo[1,5-a]pyridine-1-carboxamide

Into an 8 mL vial was placed methyl 6-(3-amino-6-chloro-2-fluorophenyl)imidazo[1,5-a]pyridine-1-carboxylate (130 mg, 0.4 mmol, 1 equiv) and 33% methylamine in ethanol solution (3 mL). The resulting solution was stirred for 24 h at 60° C. then was concentrated under reduced pressure to give crude 6-(3-amino-6-chloro-2-fluorophenyl)-N-methylimidazo[1,5-a]pyridine-1-carboxamide (100 mg) as a brown solid which was used in next step directly without further purification.

LCMS (ES, m/z): [M+H]⁺: 319

### Synthesis of Compounds 137: 6-[6-chloro-3-(5-chloro-2-methoxypyridine-3-sulfonamido)-2-fluorophenyl]-N-methylimidazo[1,5-a]pyridine-1-carboxamide

Into an 8 mL vial was placed 6-(3-amino-6-chloro-2-fluorophenyl)-N-methylimidazo[1,5-a]pyridine-1-carboxamide (100 mg, 0.3 mmol, 1 equiv), pyridine (3 mL) and 5-chloro-2-methoxypyridine-3-sulfonyl chloride (92 mg, 0.4 mmol, 1.2 equiv). The resulting solution was stirred for 2 h then concentrated under vacuum. The residue was purified by prep-HPLC with the following conditions: Column, welch Vltimate XB-C18, 50×250 mm, 10 μm; Mobile Phase: 40-80% MeCN/0.1% aqueous formic acid to afford 6-[6-chloro-3-(5-chloro-2-methoxypyridine-3-sulfonamido)-2-fluorophenyl]-N-methylimidazo[1,5-a]pyridine-1-carboxamide (45 mg, 27% yield) as a white solid.

LCMS (ES, m/z): [M+H]⁺: 524

¹H NMR (300 MHz, DMSO-d₆) δ 10.62 (s, 1H), 8.57-8.45 (m, 3H), 8.20-8.07 (m, 3H), 7.47 (d, J=9.0 Hz, 1H), 7.47-7.35 (m, 1H), 6.96 (d, J=9.3 Hz, 1H), 3.90 (s, 3H), 2.81 (d, J=4.7 Hz, 3H).

### Example 153: Synthesis of 2-[6-chloro-3-(5-chloro-2-methylpyridine-3-sulfonamido)-2-fluorophenyl]-N-methylimidazo[1,5-b]pyridazine-5-carboxamide (Compounds 138)

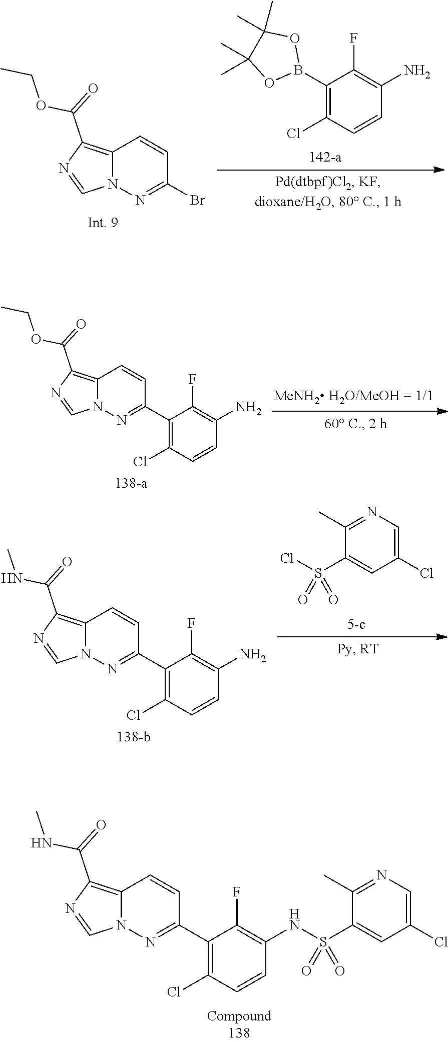 [SMILES: CCOC(=O)c1ncn2nc(Br)ccc12 ; CC1(C)OB(c2c(Cl)ccc(N)c2F)OC1(C)C ; CCOC(=O)c1ncn2nc(-c3c(Cl)ccc(N)c3F)ccc12 ; CNC(=O)c1ncn2nc(-c3c(Cl)ccc(N)c3F)ccc12 ; Cc1ncc(Cl)cc1S(=O)(=O)Cl ; CNC(=O)c1ncn2nc(-c3c(Cl)ccc(NS(=O)(=O)c4cc(Cl)cnc4C)c3F)ccc12]

### Synthesis of 138-a: methyl 6-(3-amino-6-chloro-2-fluorophenyl)imidazo[1,5-a]pyrazine-1-carboxylate

Into an 8 mL vial were added ethyl 2-bromoimidazo[1,5-b]pyridazine-5-carboxylate (200 mg, 0.74 mmol, 1 equiv), 4-chloro-2-fluoro-3-(4,4,5,5-tetramethyl-1,3,2-dioxaborolan-2-yl)aniline (301 mg, 1.1 mmol, 1.5 equiv), Pd(dtbpf)Cl₂ (48 mg, 0.07 mmol, 0.1 equiv), KF (129 mg, 2.2 mmol, 3 equiv), dioxane (4 mL) and H₂O (0.8 mL). The resulting mixture was stirred for 1 h at 80° C. under nitrogen atmosphere, then concentrated. The residue was purified by silica gel column chromatography, eluting with (PE:EA=1:5) to afford ethyl 2-(3-amino-6-chloro-2-fluorophenyl)imidazo[1,5-b]pyridazine-5-carboxylate (17 mg, 69% yield) as a light yellow solid.

LCMS (ES, m/z): [M+H]⁺: 335

### Synthesis of 138-b: 6-(3-amino-6-chloro-2-fluorophenyl)-N-methylimidazo[1,5-a]pyrazine-1-carboxamide

Into a 40 mL vial were added ethyl 2-(3-amino-6-chloro-2-fluorophenyl)imidazo[1,5-b]pyridazine-5-carboxylate (160 mg, 0.48 mmol, 1 equiv), MeOH (3 mL) and 30% methylamine in water (3 mL). The resulting mixture was stirred for 2 h at 60° C., then concentrated. The residue was purified by silica gel column chromatography, eluting with CH₂Cl₂/MeOH (10:1) to afford 2-(3-amino-6-chloro-2-fluorophenyl)-N-methylimidazo[1,5-b]pyridazine-5-carboxamide (125 mg, 82% yield) as an off-white solid.

LCMS (ES, m/z): [M+H]⁺: 320

### Synthesis of Compound 138: 2-[6-chloro-3-(5-chloro-2-methylpyridine-3-sulfonamido)-2-fluorophenyl]-N-methylimidazo[1,5-b]pyridazine-5-carboxamide

Into an 8 mL vial were added 2-(3-amino-6-chloro-2-fluorophenyl)-N-methylimidazo[1,5-b]pyridazine-5-carboxamide (115 mg, 0.36 mmol, 1 equiv), 5-chloro-2-methylpyridine-3-sulfonyl chloride (161 mg, 0.7 mmol, 2 equiv) and pyridine (5 mL). The resulting mixture was stirred for 2 h then concentrated. The crude product was purified by Prep-HPLC with the following conditions: Column: XBridge Prep C18 OBD, 5 μm, 19*150 mm; Mobile Phase: 10-35% MeCN/0.05% aqueous ammonia; to afford 2-[6-chloro-3-(5-chloro-2-methylpyridine-3-sulfonamido)-2-fluorophenyl]-N-methylimidazo[1,5-b]pyridazine-5-carboxamide (32 mg, 18% yield) as a light yellow solid.

LCMS (ES, m/z): [M+H]⁺: 509.

¹H NMR (300 MHz, DMSO-d₆) δ 11.04 (s, 1H), 8.86 (s, 1H), 8.72 (d, J=2.4 Hz, 1H), 8.58 (d, J=9.4 Hz, 1H), 8.32 (q, J=4.7 Hz, 1H), 8.13 (d, J=2.4 Hz, 1H), 7.51-7.36 (m, 2H), 7.08 (d, J=9.4 Hz, 1H), 2.82 (d, J=4.7 Hz, 3H), 2.77 (s, 3H).

### Example 154: Synthesis of 2-[2-chloro-3-(5-chloro-2-methylpyridine-3-sulfonamido)-6-fluorophenyl]-N-methylimidazo[1,5-b]pyridazine-5-carboxamide (Compound 139)

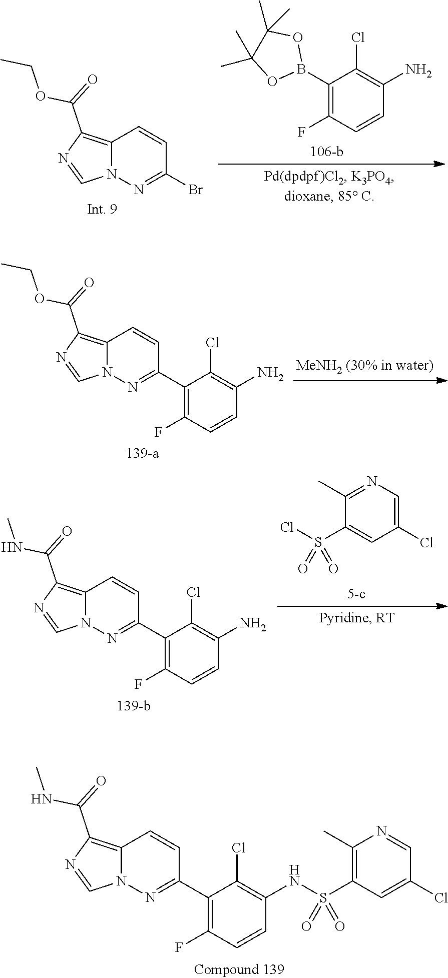 [SMILES: CCOC(=O)c1ncn2nc(Br)ccc12 ; CC1(C)OB(c2c(F)ccc(N)c2Cl)OC1(C)C ; CCOC(=O)c1ncn2nc(-c3c(F)ccc(N)c3Cl)ccc12 ; CNC(=O)c1ncn2nc(-c3c(F)ccc(N)c3Cl)ccc12 ; Cc1ncc(Cl)cc1S(=O)(=O)Cl ; CNC(=O)c1ncn2nc(-c3c(F)ccc(NS(=O)(=O)c4cc(Cl)cnc4C)c3Cl)ccc12]

### Synthesis of 139-a: ethyl 2-(3-amino-2-chloro-6-fluorophenyl)imidazo[1,5-b]pyridazine-5-carboxylate

Into a 40 mL vial purged and maintained with an inert atmosphere of nitrogen, was placed ethyl 2-bromoimidazo[1,5-b]pyridazine-5-carboxylate (180 mg, 0.67 mmol, 1 equiv), dioxane (10 mL), H₂O (2 mL), 2-chloro-4-fluoro-3-(4,4,5,5-tetramethyl-1,3,2-dioxaborolan-2-yl)aniline (217 mg, 0.8 mmol, 1.2 equiv), K₃PO₄ (283 mg, 1.3 mmol, 2 equiv) and Pd(dppf)Cl₂ (49 mg, 0.067 mmol, 0.1 equiv). The resulting solution was stirred for 2 h at 90° C. then concentrated. The crude product was purified by Flash-Prep-HPLC with the following conditions: Column, WelFlash™ C18-I, Spherical C18 20-40 m, 120 g; mobile phase: 10-80% MeCN/0.1% aqueous formic acid; to give ethyl 2-(3-amino-2-chloro-6-fluorophenyl)imidazo[1,5-b]pyridazine-5-carboxylate (120 mg, 54% yield) as a white solid.

LCMS (ES, m/z): [M+H]⁺: 335

### Synthesis of 139-b: 2-(3-amino-2-chloro-6-fluorophenyl)-N-methylimidazo[1,5-b]pyridazine-5-carboxamide

Into a 40 mL vial was placed ethyl 2-(3-amino-2-chloro-6-fluorophenyl)imidazo[1,5-b]pyridazine-5-carboxylate (180 mg, 0.54 mmol, 1 equiv), MeOH (10 mL) and 30% aqueous methylamine (3 mL). The resulting solution was stirred overnight at 45° C. then concentrated to give 2-(3-amino-2-chloro-6-fluorophenyl)-N-methylimidazo[1,5-b]pyridazine-5-carboxamide (120 mg, 70% yield) as a white solid.

LCMS (ES, m/z): [M+H]⁺: 320

### Synthesis of Compound 139: 2-[2-chloro-3-(5-chloro-2-methylpyridine-3-sulfonamido)-6-fluorophenyl]-N-methylimidazo[1,5-b]pyridazine-5-carboxamide

Into a 40 mL vial was placed 2-(3-amino-2-chloro-6-fluorophenyl)-N-methylimidazo[1,5-b]pyridazine-5-carboxamide (120 mg, 0.38 mmol, 1 equiv), pyridine (5 mL) and 5-chloro-2-methylpyridine-3-sulfonyl chloride (127 mg, 0.56 mmol, 1.5 equiv). The resulting solution was stirred for 2 days then concentrated. The crude product was purified by Prep-HPLC with the following conditions: Column, welch Vltimate XB-C18, 50×250 mm, 10 μm; Mobile Phase: 10-65% MeCN/0.1% aqueous ammonia; to give 2-[2-chloro-3-(5-chloro-2-methylpyridine-3-sulfonamido)-6-fluorophenyl]-N-methylimidazo[1,5-b]pyridazine-5-carboxamide (40 mg, 21% yield) as a light yellow solid.

LCMS (ES, m/z): [M+H]⁺: 509

¹H NMR (300 MHz, DMSO-d₆) δ 10.84 (br s, 1H), 8.86 (s, 1H), 8.75 (d, J=2.4 Hz, 1H), 8.58 (d, J=9.4 Hz, 1H), 8.32 (q, J=4.7 Hz, 1H), 8.04 (d, J=2.4 Hz, 1H), 7.51 (dd, J=9.1, 5.7 Hz, 1H), 7.44 (t, J=8.8 Hz, 1H), 7.06 (d, J=9.4 Hz, 1H), 2.86 (d, 3H), 2.74 (s, 3H).

### Example 155: Synthesis of 6-(2-fluoro-5-((5-fluoro-2-methoxypyridine)-3-sulfonamido)phenyl)-N-methyl-5,6,7,8-tetrahydroimidazo[1,5-a]pyridine-1-carboxamide (Compound 140)

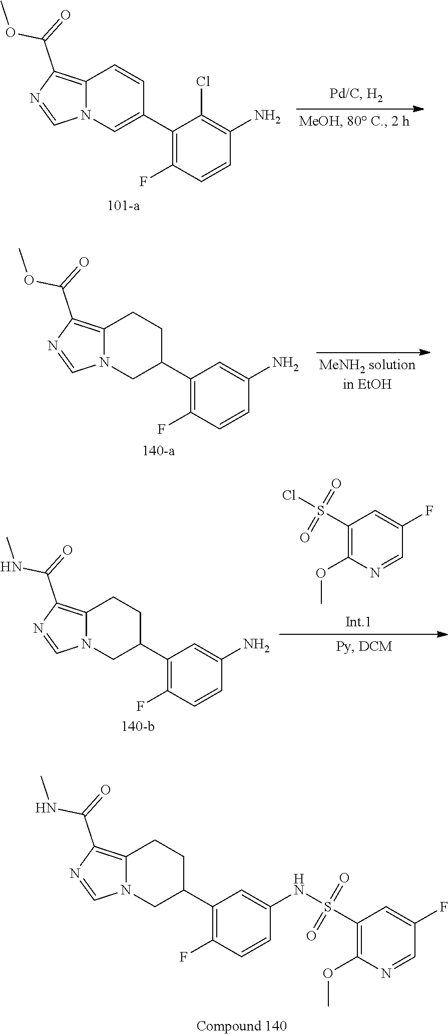 [SMILES: COC(=O)c1ncn2cc(-c3c(F)ccc(N)c3Cl)ccc12 ; COC(=O)c1ncn2c1CCC(c1cc(N)ccc1F)C2 ; CNC(=O)c1ncn2c1CCC(c1cc(N)ccc1F)C2 ; COc1ncc(F)cc1S(=O)(=O)Cl ; CNC(=O)c1ncn2c1CCC(c1cc(NS(=O)(=O)c3cc(F)cnc3OC)ccc1F)C2]

### Synthesis of 140-a: methyl 6-(5-amino-2-fluorophenyl)-5,6,7,8-tetrahydroimidazo[1,5-a]pyridine-1-carboxylate

To a solution of methyl 6-(3-amino-2-chloro-6-fluorophenyl)imidazo[1,5-a]pyridine-1-carboxylate (100 mg, 0.3 mmol, 1 equiv) in 10 mL MeOH was added Pd/C (10%, 66 mg) in a pressure tank. The mixture was hydrogenated at room temperature under 20 atm of hydrogen pressure for 2 h at 80° C., then filtered through a Celite pad. The filtrate was concentrated and the residue was purified by silica gel column chromatography, eluting with PE:EA (1:1) to afford methyl 6-(5-amino-2-fluorophenyl)-5,6,7,8-imidazo[1,5-a]pyridine-1-carboxylate (60 mg, 66% yield) as a yellow oil.

LCMS (ES, m/z): [M+H]⁺: 290

### Synthesis of 140-b: 6-(5-amino-2-fluorophenyl)-N-methyl-5,6,7,8-imidazo[1,5-a]pyridine-1-carboxamide

To a solution of methyl 6-(5-amino-2-fluorophenyl)-5,6,7,8-imidazo[1,5-a]pyridine-1-carboxylate (60 mg) and CH₃NH₂ solution in MeOH (5 mL, 2 M) was stirred at 80° C. for 20 h. The mixture was cooled and concentrated to give crude 6-(5-amino-2-fluorophenyl)-N-methyl-5H,6H,7H,8H-imidazo[1,5-a]pyridine-1-carboxamide (60 mg).

LCMS (ES, m/z): [M+H]⁺: 289

### Synthesis of Compound 140: 6-(2-fluoro-5-((5-fluoro-2-methoxypyridine)-3-sulfonamido)phenyl)-N-methyl-5,6,7,8-tetrahydroimidazo[1,5-a]pyridine-1-carboxamide

To a stirred solution of 6-(5-amino-2-fluorophenyl)-N-methyl-5,6,7,8-imidazo[1,5-a]pyridine-1-carboxamide (60 mg, 0.2 mmol, 1 equiv) and pyridine (49 mg, 0.6 mmol, 3 equiv) in DCM (5 mL) was added 5-fluoro-2-methoxypyridine-3-sulfonyl chloride (70 mg, 0.3 mmol, 1.5 equiv). The reaction was stirred for 1 h then concentrated. The residue was purified by prep-HPLC with following conditions: Column, welch Vltimate XB-C18, 50×250 mm, 10 m; Mobile Phase: 15-55% MeCN/0.1% aqueous formic acid; to afford 6-(2-fluoro-5-((5-fluoro-2-methoxypyridine)-3-sulfonamido)phenyl)-N-methyl-5,6,7,8-tetrahydroimidazo[1,5-a]pyridine-1-carboxamide (60 mg, 60% yield) as a white solid.

LCMS (ES, m/z): [M+H]⁺: 478

¹H NMR (300 MHz, Chloroform-d) δ 8.19 (d, J=3.0 Hz, 1H), 7.83 (dd, J=6.8, 3.0 Hz, 1H), 7.34 (s, 1H), 7.26 (s, 1H), 7.10-7.01 (m, 2H), 6.98 (d, J=7.8 Hz, 2H), 4.28 (dd, J=12.2, 5.0 Hz, 1H), 4.16 (s, 3H), 3.86 (q, J=11.8 Hz, 1H), 3.64-3.51 (m, 1H), 3.46 (d, J=11.4 Hz, 1H), 3.07 (td, J=11.4, 5.6 Hz, 1H), 2.98 (d, J=4.8 Hz, 3H), 2.20-1.97 (m, 2H).

### Example 156: Synthesis of 6-(6-chloro-3-((5-chloro-2-methoxypyridine)-3-sulfonamido)-2-fluorophenyl)-N-methylimidazo[1,5-a]pyrazine-1-carboxamide (Compound 141)

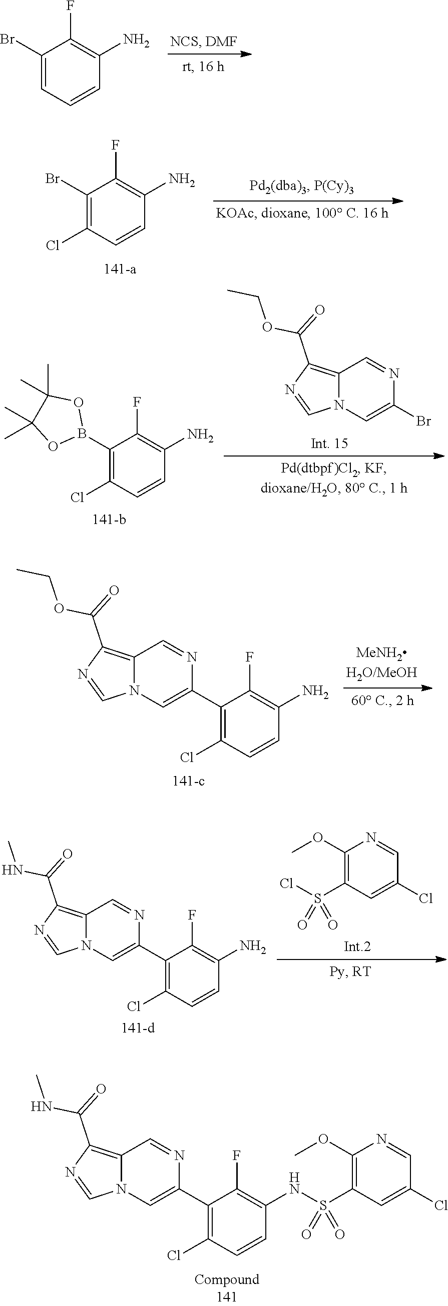

### Synthesis of 141-a: 3-bromo-4-chloro-2-fluoroaniline

To a stirred solution of 3-bromo-2-fluoroaniline (5 g, 26 mmol, 1 equiv) in DMF (25 mL) was added NCS (3.69 g, 28 mmol, 1.05 equiv) in portions at 0° C. The reaction was stirred overnight then quenched by the addition of water/ice (25 mL). The resulting mixture was extracted with EtOAc (3×100 mL) and the combined organics washed with water and brine, then dried over anhydrous Na₂SO₄ and concentrated. The residue was purified by silica gel column chromatography, eluting with EtOAc/PE (0-30%) to afford 3-bromo-4-chloro-2-fluoroaniline (3 g, 51% yield) as a yellow solid.

LCMS (ES, m/z): [M+H]⁺: 224, 226

### Synthesis of 141-b: 4-chloro-2-fluoro-3-(4,4,5,5-tetramethyl-1,3,2-dioxaborolan-2-yl)aniline

A solution of 3-bromo-4-chloro-2-fluoroaniline (200 mg, 0.89 mmol, 1 equiv), bis(pinacolato)diboron (339 mg, 1.3 mmol, 1.5 equiv), tricyclohexylphosphine (50 mg, 0.18 mmol, 0.2 equiv), Pd₂(dba)₃ (81 mg, 0.09 mmol, 0.1 equiv) and KOAc (175 mg, 1.8 mmol, 2 equiv) in dioxane (2 mL) was stirred for 16 h at 100° C. under nitrogen atmosphere. The mixture was cooled and diluted with water (10 mL), then extracted with EtOAc (3×50 mL).

The combined organics were washed with water and brine, dried over anhydrous Na₂SO₄ and concentrated. The residue was purified by silica gel column chromatography, eluting with EtOAc/PE (0-50%) to afford 4-chloro-2-fluoro-3-(4,4,5,5-tetramethyl-1,3,2-dioxaborolan-2-yl)aniline (150 mg, 62% yield) as a yellow solid.

LCMS (ES, m/z): [M+H]⁺: 272.

### Synthesis of 141-c: methyl 6-(3-amino-6-chloro-2-fluorophenyl)imidazo[1,5-a]pyrazine-1-carboxylate

Into an 8 mL vial were added ethyl 6-bromoimidazo[1,5-a]pyrazine-1-carboxylate (200 mg, 0.74 mmol, 1 equiv), 4-chloro-2-fluoro-3-(4,4,5,5-tetramethyl-1,3,2-dioxaborolan-2-yl) aniline (295 mg, 1.1 mmol, 1.5 equiv), Pd(dtbpf)Cl₂ (53 mg, 0.08 mmol, 0.1 equiv), KF (126 mg, 2.2 mmol, 3 equiv), dioxane (4 mL) and H₂O (0.8 mL). The resulting mixture was stirred for 1 h at 80° C. under nitrogen atmosphere, then concentrated. The residue was purified by silica gel column chromatography, eluting with (PE:EA=1:5) to afford methyl 6-(3-amino-6-chloro-2-fluorophenyl)imidazo[1,5-a]pyrazine-1-carboxylate (160 mg, 67% yield) as a light yellow solid.

LCMS (ES, m/z): [M+H]⁺: 335.

### Synthesis of 141-d: 6-(3-amino-6-chloro-2-fluorophenyl)-N-methylimidazo[1,5-a]pyrazine-1-carboxamide

Into a 40 mL vial were added methyl 6-(3-amino-6-chloro-2-fluorophenyl)imidazo[1,5-a]pyrazine-1-carboxylate (160 mg, 0.5 mmol, 1 equiv), MeOH (3 mL) and 30% aqueous methylamine (3 mL). The resulting mixture was stirred for 2 h at 60° C., then concentrated. The residue was purified by silica gel column chromatography, eluting with CH₂Cl₂/MeOH (10:1) to afford 6-(3-amino-6-chloro-2-fluorophenyl)-N-methylimidazo[1,5-a]pyrazine-1-carboxamide (110 mg, 69% yield) as an off-white solid.

LCMS (ES, m/z): [M+H]⁺: 320.

### Synthesis of Compound 141: 6-(6-chloro-3-((5-chloro-2-methoxypyridine)-3-sulfonamido)-2-fluorophenyl)-N-methylimidazo[1,5-a]pyrazine-1-carboxamide

Into an 8 mL vial were added 6-(3-amino-6-chloro-2-fluorophenyl)-N-methylimidazo[1,5-a]pyrazine-1-carboxamide (100 mg, 0.31 mmol, 1 equiv), 5-chloro-2-methoxypyridine-3-sulfonyl chloride (150 mg, 0.62 mmol, 2 equiv) and pyridine (5 mL). The resulting mixture was stirred for 2 h then concentrated. The crude product was purified by Prep-HPLC with the following conditions: Column: XBridge Prep C18 OBD, 5 μm, 19*150 mm; Mobile Phase: 10-35% MeCN/0.05% aqueous ammonia; Flow rate: 20 mL/min; to give 6-[6-chloro-3-(5-chloro-2-methoxypyridine-3-sulfonamido)-2-fluorophenyl]-N-methylimidazo[1,5-a]pyrazine-1-carboxamide (61 mg, 37% yield) as an off-white solid.

LCMS (ES, m/z): [M+H]⁺: 525.

¹H NMR (300 MHz, DMSO-d₆) δ 10.62 (s, 1H), 9.52 (d, J=1.7 Hz, 1H), 8.69-8.60 (m, 2H), 8.50 (d, J=2.6 Hz, 1H), 8.43 (d, J=5.0 Hz, 1H), 8.13 (d, J=2.6 Hz, 1H), 7.44 (d, J=3.9 Hz, 2H), 3.90 (s, 3H), 2.85 (d, J=4.7 Hz, 3H).

### Example 157: Synthesis of N-[4-chloro-2-fluoro-3-[1-(1H-imidazol-2-yl)imidazo[1,5-a]pyrazin-6-yl]phenyl]-5-fluoro-2-methoxypyridine-3-sulfonamide (Compound 142)

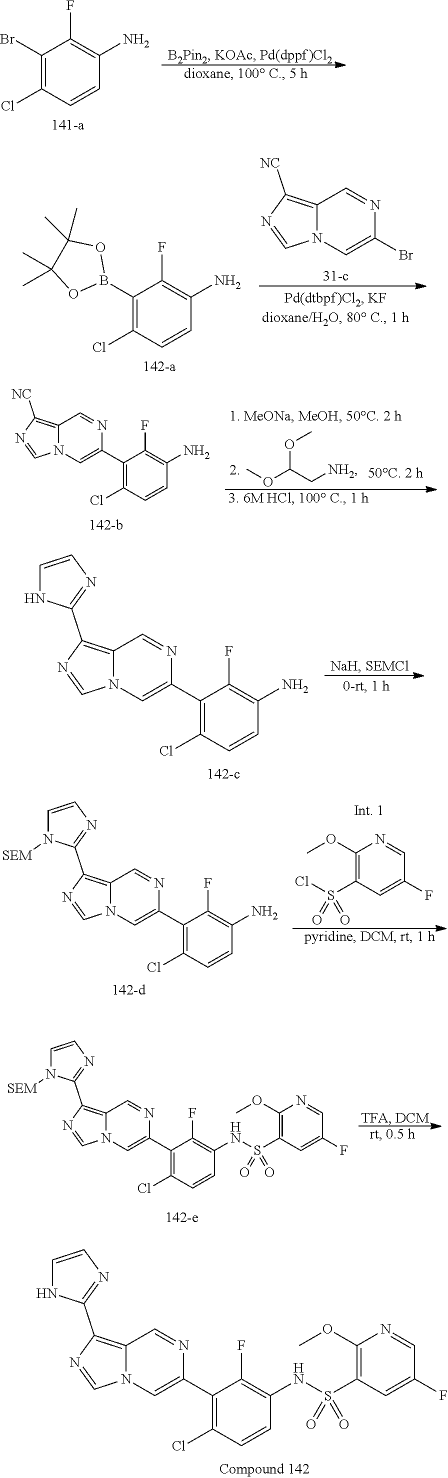

### Synthesis of 142-a: 4-chloro-2-fluoro-3-(4,4,5,5-tetramethyl-1,3,2-dioxaborolan-2-yl)aniline

A solution of 3-bromo-4-chloro-2-fluoroaniline (200 mg, 0.89 mmol, 1 equiv), bis(pinacolato)diboron (340 mg, 1.34 mmol, 1.5 equiv), tricyclohexylphosphine (50 mg, 0.18 mmol, 0.2 equiv), Pd₂(dba)₃ (82 mg, 0.09 mmol, 0.1 equiv) and KOAc (175 mg, 1.78 mmol, 2 equiv) in dioxane (2 mL) was stirred for 5 h at 100° C. under nitrogen atmosphere. The mixture was cooled and diluted with water (10 mL), then was extracted with EtOAc (3×50 mL). The combined organics were washed with H₂O (50 mL) and brine (50 mL), dried over anhydrous Na₂SO₄ and concentrated. The residue was purified by silica gel column chromatography, eluting with PE/EtOAc (2/1) to afford 4-chloro-2-fluoro-3-(4,4,5,5-tetramethyl-1,3,2-dioxaborolan-2-yl)aniline (150 mg, 62% yield) as a yellow solid.

LCMS (ES, m/z): [M+H]⁺: 272

### Synthesis of 142-b: 6-(3-amino-6-chloro-2-fluorophenyl)imidazo[1,5-a]pyrazine-1-carbonitrile

To a stirred solution of 6-bromoimidazo[1,5-a]pyrazine-1-carbonitrile (510 mg, 2.3 mmol, 1 equiv) and 4-chloro-2-fluoro-3-(4,4,5,5-tetramethyl-1,3,2-dioxaborolan-2-yl)aniline (930 mg, 3.4 mmol, 1.5 equiv) in dioxane (5 mL) and H₂O (0.5 mL) were added KF (399 mg, 6.9 mmol, 3 equiv) and Pd(dtbpf)Cl₂ (149 mg, 0.23 mmol, 0.1 equiv) in portions at room temperature under nitrogen atmosphere. The resulting mixture was stirred for 1 h at 80° C. then quenched with H₂O (20 mL) and extracted with EA (3×10 mL). The combined organics were washed with brine (20 mL), dried over anhydrous Na₂SO₄ and concentrated. The residue was purified by Flash-Prep-HPLC with the following conditions: Column, WelFlash™ C18-I, Spherical C18 20-40 m, 120 g; mobile phase: 30-80% MeCN/0.1% aqueous formic acid; to afford 6-(3-amino-6-chloro-2-fluorophenyl)imidazo[1,5-a]pyrazine-1-carbonitrile (607 mg, 92% yield) as a light yellow solid.

LCMS (ES, m/z): [M+H]⁺: 288

### Synthesis of 142-c: 4-chloro-2-fluoro-3-[1-(1H-imidazol-2-yl)imidazo[1,5-a]pyrazin-6-yl]aniline

To a stirred solution of 6-(3-amino-6-chloro-2-fluorophenyl)imidazo[1,5-a]pyrazine-1-carbonitrile (300 mg, 1 mmol, 1 equiv) in MeOH (5 mL) in an ice bath was added MeONa (225 mg, 4.2 mmol, 4 equiv) in portions. The resulting mixture was stirred for 2 h at 50° C., then cooled to room temperature. 2,2-Dimethoxyethanamine (132 mg, 1.3 mmol, 1.2 equiv) was added dropwise over 2 min then this was stirred for 2 h at 50° C. The mixture was allowed to cool and 6 M aqueous HCl (8.8 mL) was added dropwise over 2 min. The resulting mixture was stirred for 1 h at 100° C. H₂O (10 mL) was added and the mixture extracted with EA (3×5 mL). The combined organics were washed with brine (10 mL), dried over anhydrous Na₂SO₄ and concentrated. The residue was purified by Flash-Prep-HPLC with the following conditions: Column, WelFlash™ C18-I, Spherical C18 20-40 m, 120 g; mobile phase: 20-70% MeCN/0.1% aqueous formic acid; to afford 4-chloro-2-fluoro-3-[1-(1H-imidazol-2-yl)imidazo[1,5-a]pyrazin-6-yl]aniline (66 mg, 19% yield) as a yellow solid.

LCMS (ES, m/z): [M+H]⁺: 329

### Synthesis of 142-d: 4-chloro-2-fluoro-3-[1-(1-[[2-(trimethylsilyl)ethoxy]methyl]imidazol-2-yl)imidazo[1,5-a]pyrazin-6-yl]aniline

To a stirred solution of 4-chloro-2-fluoro-3-[1-(1H-imidazol-2-yl)imidazo[1,5-a]pyrazin-6-yl]aniline (75 mg, 0.23 mmol, 1 equiv) in THE (4 mL) was added 60% NaH in oil (16 mg, 0.68 mmol, 3 equiv) in portions at 0° C. The resulting mixture was stirred for 0.5 h at room temperature then SEMCI (57 mg, 0.34 mmol, 1.5 equiv) was added dropwise at 0° C. The resulting mixture was stirred for 1 h at room temperature, before H₂O (5 mL) was added. This was extracted with EA (3×3 mL), and the combined organics were washed with brine (5 mL), dried over anhydrous Na₂SO₄ then concentrated. The residue was purified by Flash-Prep-HPLC with the following conditions: Column, WelFlash™ C18-I, Spherical C18 20-40 m, 120 g; mobile phase: 20-70% MeCN/0.1% aqueous formic acid; to afford 4-chloro-2-fluoro-3-[1-(1-[[2-(trimethylsilyl)ethoxy]methyl]imidazol-2-yl)imidazo[1,5-a]pyrazin-6-yl]aniline (54 mg, 52% yield) as a brown oil.

LCMS (ES, m/z): [M+H]⁺: 459

### Synthesis of 142-e: N-[4-chloro-2-fluoro-3-[1-(1-[[2-(trimethylsilyl)ethoxy]methyl]imidazol-2-yl)imidazo[1,5-a]pyrazin-6-yl]phenyl-5-fluoro-2-methoxypyridine-3-sulfonamide

To a stirred solution of 4-chloro-2-fluoro-3-[1-(1-[[2-(trimethylsilyl)ethoxy]methyl]imidazol-2-yl)imidazo[1,5-a]pyrazin-6-yl]aniline (50 mg, 0.11 mmol, 1 equiv) in pyridine (2 mL) was added 5-fluoro-2-methoxypyridine-3-sulfonyl chloride (49 mg, 0.22 mmol, 2 equiv) in DCM (0.5 mL) dropwise. The resulting mixture was stirred for 1 h then concentrated to give crude N-[4-chloro-2-fluoro-3-[1-(1-[[2-(trimethylsilyl)ethoxy]methyl]imidazol-2-yl)imidazo[1,5-a]pyrazin-6-yl]phenyl]-5-fluoro-2-methoxypyridine-3-sulfonamide as a yellow oil, which was used in next step directly without further purification.

LCMS (ES, m/z): [M+H]⁺: 648

### Synthesis of Compound 142: N-[4-chloro-2-fluoro-3-[1-(1H-imidazol-2-yl)imidazo[1,5-a]pyrazin-6-yl]phenyl]-5-fluoro-2-methoxypyridine-3-sulfonamide

Into a 40 mL vial was placed N-[4-chloro-2-fluoro-3-[1-(1-[[2-(trimethylsilyl)ethoxy]methyl]imidazol-2-yl)imidazo[1,5-a]pyrazin-6-yl]phenyl]-5-fluoro-2-methoxypyridine-3-sulfonamide (50 mg, 0.08 mmol, 1 equiv), DCM (1.5 mL) and TFA (0.5 mL). The resulting solution was stirred for 0.5 h then concentrated. The residue was purified by prep-HPLC with the following conditions: Column, welch Vltimate XB-C18, 50×250 mm, 10 μm; Mobile Phase: 20-60% MeCN/0.1% aqueous formic acid; to afford N-[4-chloro-2-fluoro-3-[1-(1H-imidazol-2-yl)imidazo[1,5-a]pyrazin-6-yl]phenyl]-5-fluoro-2-methoxypyridine-3-sulfonamide (20 mg, 50% yield) as a yellow solid.

LCMS (ES, m/z): [M+H]⁺: 518

### Example 158: Synthesis of 5-chloro-N-(3-(1-(4,5-dimethyl-1H-imidazol-2-yl)imidazo[1,5-a]pyridin-6-yl)-2,4-difluorophenyl)-2-methoxypyridine-3-sulfonamide (Compound 143)

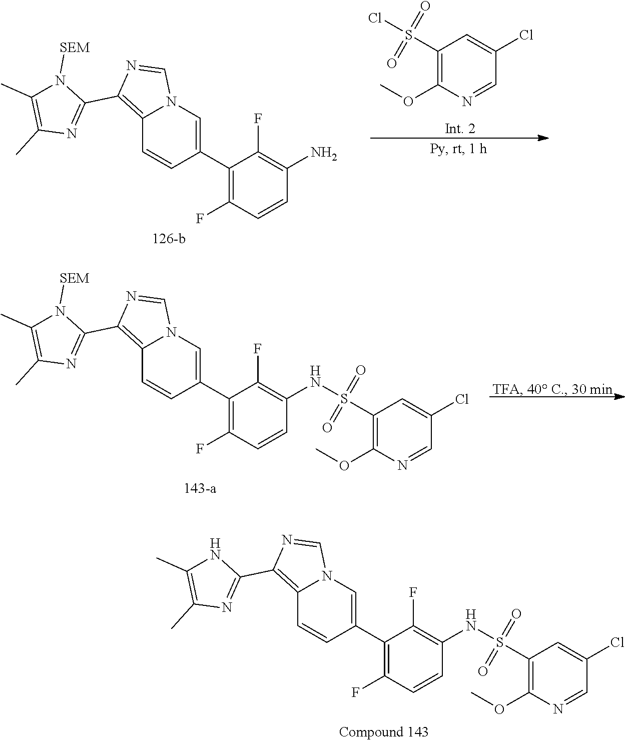 [SMILES: Cc1nc(-c2ncn3cc(-c4c(F)ccc(N)c4F)ccc23)n(COCC[Si](C)(C)C)c1C ; COc1ncc(Cl)cc1S(=O)(=O)Cl ; COc1ncc(Cl)cc1S(=O)(=O)Nc1ccc(F)c(-c2ccc3c(-c4nc(C)c(C)n4COCC[Si](C)(C)C)ncn3c2)c1F ; COc1ncc(Cl)cc1S(=O)(=O)Nc1ccc(F)c(-c2ccc3c(-c4nc(C)c(C)[nH]4)ncn3c2)c1F]

### Synthesis of 143-a: 5-chloro-N-(3-(1-(4,5-dimethyl-1-((2-(trimethylsilyl)ethoxy)methyl)-1H-imidazol-2-yl) imidazo[1,5-a]pyridin-6-yl)-2,4-difluorophenyl)-2-methoxypyridine-3-sulfonamide

Into an 8 mL vial were added 3-[1-(4,5-dimethyl-1-[[2-(trimethylsilyl)ethoxy]methyl]imidazol-2-yl)imidazo[1,5-a]pyridin-6-yl]-2,4-difluoroaniline (150 mg, 0.32 mmol, 1 equiv), 5-chloro-2-methoxypyridine-3-sulfonyl chloride (155 mg, 0.64 mmol, 2 equiv) and pyridine (3 mL). The resulting mixture was stirred for 2 h then concentrated. The residue was purified by silica gel column chromatography, eluting with PE/EtOAc (10:7) to afford 5-chloro-N-[3-[1-(4,5-dimethyl-1-[[2-(trimethylsilyl)ethoxy]methyl]imidazol-2-yl)imidazo[1,5-a]pyridin-6-yl]-2,4-difluorophenyl]-2-methoxypyridine-3-sulfonamide (140 mg, 65% yield) as a light yellow solid.

LCMS (ES, m/z): [M+H]⁺: 675.

### Synthesis of Compound 143: 5-chloro-N-(3-(1-(4,5-dimethyl-1H-imidazol-2-yl)imidazo[1,5-a]pyridin-6-yl)-2,4-difluorophenyl)-2-methoxypyridine-3-sulfonamide

Into an 8 mL vial were added 5-chloro-N-[3-[1-(4,5-dimethyl-1-[[2-(trimethylsilyl)ethoxy]methyl]imidazol-2-yl)imidazo[1,5-a]pyridin-6-yl]-2,4-difluorophenyl]-2-methoxypyridine-3-sulfonamide (130 mg, 0.19 mmol, 1 equiv) and TFA (3 mL). The resulting mixture was stirred for 30 min at 40° C., then concentrated. The crude product was purified by Prep-HPLC with the following conditions: XBridge Prep C18 OBD Column, 5 μm, 19*150 mm; Mobile Phase: 24-41% MeCN/0.05% aqueous ammonia; to give 5-chloro-N-[3-[1-(4,5-dimethyl-1H-imidazol-2-yl)imidazo[1,5-a]pyridin-6-yl]-2,4-difluorophenyl]-2-methoxypyridine-3-sulfonamide (17 mg, 17% yield) as a yellow solid.

LCMS (ES, m/z): [M+H]⁺: 545.

¹H NMR (300 MHz, DMSO-d₆) δ 11.33 (s, 1H), 8.51-8.44 (m, 3H), 8.19 (d, J=9.4 Hz, 1H), 8.07 (d, J=2.6 Hz, 1H), 7.35 (td, J=8.9, 5.9 Hz, 1H), 7.19 (td, J=9.0, 1.5 Hz, 1H), 6.81 (d, J=9.4 Hz, 1H), 3.91 (s, 3H), 2.14 (s, 6H).

### Example 159: Synthesis of N-(3-(1-(4,5-dimethyl-1H-imidazol-2-yl)imidazo[1,5-a]pyridin-6-yl)-2,4-difluorophenyl)-5-fluoro-2-methoxypyridine-3-sulfonamide (Compound 144)

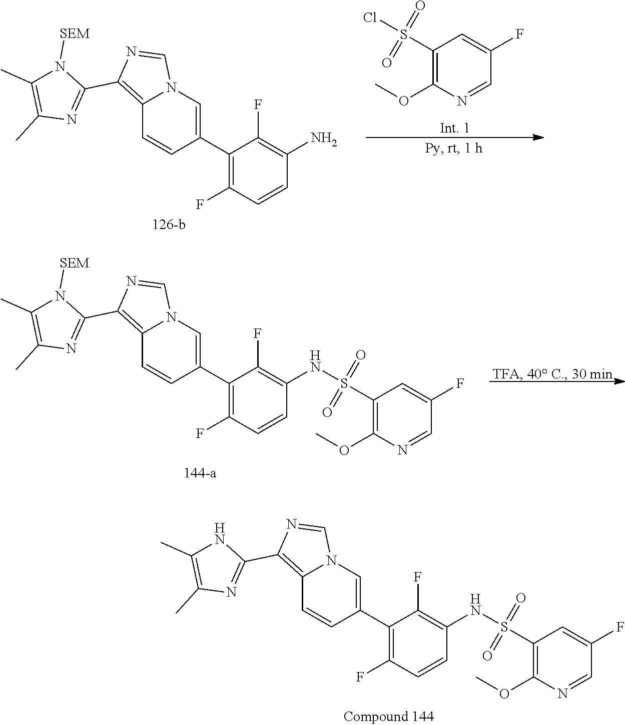 [SMILES: Cc1nc(-c2ncn3cc(-c4c(F)ccc(N)c4F)ccc23)n(COCC[Si](C)(C)C)c1C ; COc1ncc(F)cc1S(=O)(=O)Cl ; COc1ncc(F)cc1S(=O)(=O)Nc1ccc(F)c(-c2ccc3c(-c4nc(C)c(C)n4COCC[Si](C)(C)C)ncn3c2)c1F ; COc1ncc(F)cc1S(=O)(=O)Nc1ccc(F)c(-c2ccc3c(-c4nc(C)c(C)[nH]4)ncn3c2)c1F]

### Synthesis of 144-a: N-(3-(1-(4,5-dimethyl-1-((2-(trimethylsilyl)ethoxy)methyl)-1H-imidazol-2-yl)imidazo[1,5-a]pyridin-6-yl)-2,4-difluorophenyl)-5-fluoro-2-methoxypyridine-3-sulfonamide

Into an 8 mL vial were added 3-[1-(4,5-dimethyl-1-[[2-(trimethylsilyl)ethoxy]methyl]imidazol-2-yl)imidazo[1,5-a]pyridin-6-yl]-2,4-difluoroaniline (150 mg, 0.32 mmol, 1 equiv), 5-fluoro-2-methoxypyridine-3-sulfonyl chloride (144 mg, 0.64 mmol, 2 equiv) and pyridine (3 mL). The resulting mixture was stirred for 2 h then concentrated. The residue was purified by silica gel column chromatography, eluting with PE/EtOAc (10:7) to afford N-[3-[1-(4,5-dimethyl-1-[[2-(trimethylsilyl)ethoxy]methyl]imidazol-2-yl)imidazo[1,5-a]pyridin-6-yl]-2,4-difluorophenyl]-5-fluoro-2-methoxypyridine-3-sulfonamide (130 mg, 62% yield) as a light yellow solid.

LCMS (ES, m/z): [M+H]⁺: 659.

### Synthesis of Compound 144: N-(3-(1-(4,5-dimethyl-1H-imidazol-2-yl)imidazo[1,5-a]pyridin-6-yl)-2,4-difluorophenyl)-5-fluoro-2-methoxypyridine-3-sulfonamide

Into an 8 mL vial were added N-[3-[1-(4,5-dimethyl-1-[[2-(trimethylsilyl)ethoxy]methyl]imidazol-2-yl)imidazo[1,5-a]pyridin-6-yl]-2,4-difluorophenyl]-5-fluoro-2-methoxypyridine-3-sulfonamide (120 mg, 0.18 mmol, 1 equiv) and TFA (3 mL). The resulting mixture was stirred for 30 min at 40° C., then concentrated. The crude product was purified by Prep-HPLC with the following conditions: XBridge Prep C18 OBD Column, 5 μm, 19*150 mm; Mobile Phase: 24-41% MeCN/0.05% aqueous ammonia; to give N-[3-[1-(4,5-dimethyl-1H-imidazol-2-yl)imidazo[1,5-a]pyridin-6-yl]-2,4-difluorophenyl]-5-fluoro-2-methoxypyridine-3-sulfonamide (32 mg, 33% yield) as a yellow solid.

LCMS (ES, m/z): [M+H]⁺: 529.

¹H NMR (300 MHz, DMSO-d₆) δ 11.41-11.35 (m, 2H), 8.51-8.41 (m, 3H), 8.19 (d, J=9.4 Hz, 1H), 8.02 (dd, J=7.4, 3.0 Hz, 1H), 7.35 (td, J=8.9, 5.9 Hz, 1H), 7.20 (td, J=9.1, 1.5 Hz, 1H), 6.80 (d, J=9.4 Hz, 1H), 3.91 (s, 3H), 2.14 (s, 6H).

### Example 160: Synthesis of N-(3-(1-(4,5-dimethyl-1H-imidazol-2-yl)imidazo[1,5-a]pyridin-6-yl)-2,4-difluorophenyl)-5-fluoro-2-methylpyridine-3-sulfonamide (Compound 145)

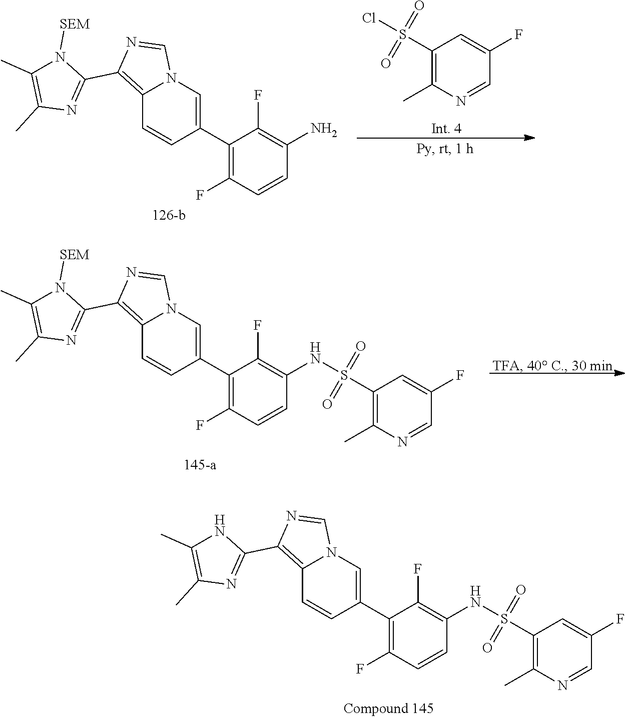 [SMILES: Cc1nc(-c2ncn3cc(-c4c(F)ccc(N)c4F)ccc23)n(COCC[Si](C)(C)C)c1C ; Cc1ncc(F)cc1S(=O)(=O)Cl ; Cc1ncc(F)cc1S(=O)(=O)Nc1ccc(F)c(-c2ccc3c(-c4nc(C)c(C)n4COCC[Si](C)(C)C)ncn3c2)c1F ; Cc1ncc(F)cc1S(=O)(=O)Nc1ccc(F)c(-c2ccc3c(-c4nc(C)c(C)[nH]4)ncn3c2)c1F]

### Synthesis of 145-a: N-(3-(1-(4,5-dimethyl-1-((2-(trimethylsilyl)ethoxy)methyl)-1H-imidazol-2-yl)imidazo[1,5-a]pyridin-6-yl)-2,4-difluorophenyl)-5-fluoro-2-methylpyridine-3-sulfonamide

Into an 8 mL vial were added 3-[1-(4,5-dimethyl-1-[[2-(trimethylsilyl)ethoxy]methyl]imidazol-2-yl)imidazo[1,5-a]pyridin-6-yl]-2,4-difluoroaniline (150 mg, 0.32 mmol, 1 equiv), 5-fluoro-2-methylpyridine-3-sulfonyl chloride (134 mg, 0.64 mmol, 2 equiv) and pyridine (3 mL). The resulting mixture was stirred for 2 h then concentrated. The residue was purified by silica gel column chromatography, eluting with PE/EtOAc (10:7) to afford N-[3-[1-(4,5-dimethyl-1-[[2-(trimethylsilyl)ethoxy]methyl]imidazol-2-yl)imidazo[1,5-a]pyridin-6-yl]-2,4-difluorophenyl]-5-fluoro-2-methylpyridine-3-sulfonamide (150 mg, 73% yield) as a light yellow solid.

LCMS (ES, m/z): [M+H]⁺: 643.

### Synthesis of Compound 145: N-(3-(1-(4,5-dimethyl-1H-imidazol-2-yl)imidazo[1,5-a]pyridin-6-yl)-2,4-difluorophenyl)-5-fluoro-2-methylpyridine-3-sulfonamide

Into an 8 mL vial were added N-[3-[1-(4,5-dimethyl-1-[[2-(trimethylsilyl)ethoxy]methyl]imidazol-2-yl)imidazo[1,5-a]pyridin-6-yl]-2,4-difluorophenyl]-5-fluoro-2-methylpyridine-3-sulfonamide (140 mg, 0.22 mmol, 1 equiv) and TFA (3 mL). The resulting mixture was stirred for 30 min at 40° C., then concentrated. The crude product was purified by Prep-HPLC with the following conditions: XBridge Prep C18 OBD Column, 5 μm, 19*150 mm; Mobile Phase: 24-41% MeCN/0.05% aqueous ammonia; Flow rate: 20 mL/min; to give N-[3-[1-(4,5-dimethyl-1H-imidazol-2-yl)imidazo[1,5-a]pyridin-6-yl]-2,4-difluorophenyl]-5-fluoro-2-methylpyridine-3-sulfonamide (41 mg, 37% yield) as a yellow solid.

LCMS (ES, m/z): [M+H]⁺: 513.

¹H NMR (300 MHz, DMSO-d₆) δ 8.60 (d, J=2.9 Hz, 1H), 8.50 (d, J=6.0 Hz, 2H), 8.18 (d, J=9.5 Hz, 1H), 7.93 (dd, J=8.4, 2.9 Hz, 1H), 7.27 (td, J=9.1, 6.0 Hz, 1H), 7.07 (td, J=9.1, 1.6 Hz, 1H), 6.87 (d, J=9.4 Hz, 1H), 2.78 (d, J=1.2 Hz, 3H), 2.16 (s, 6H).

### Example 161: Synthesis of 5-chloro-N-(3-(1-(4,5-dimethyl-1H-imidazol-2-yl)imidazo[1,5-a]pyridin-6-yl)-2,4-difluorophenyl)-2-methylpyridine-3-sulfonamide (Compound 146)

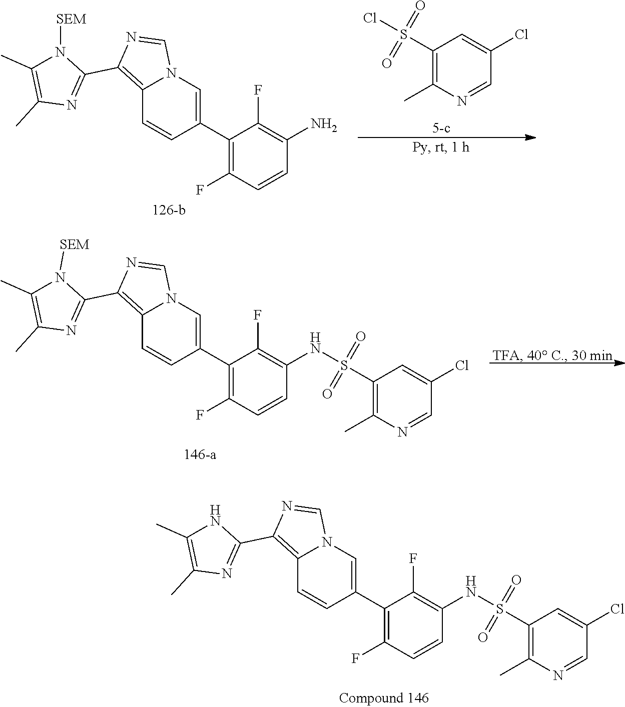 [SMILES: Cc1nc(-c2ncn3cc(-c4c(F)ccc(N)c4F)ccc23)n(COCC[Si](C)(C)C)c1C ; Cc1ncc(Cl)cc1S(=O)(=O)Cl ; Cc1ncc(Cl)cc1S(=O)(=O)Nc1ccc(F)c(-c2ccc3c(-c4nc(C)c(C)n4COCC[Si](C)(C)C)ncn3c2)c1F ; Cc1ncc(Cl)cc1S(=O)(=O)Nc1ccc(F)c(-c2ccc3c(-c4nc(C)c(C)[nH]4)ncn3c2)c1F]

### Synthesis of 146-a: 5-chloro-N-(3-(1-(4,5-dimethyl-1-((2-(trimethylsilyl)ethoxy)methyl)-1H-imidazol-2-yl)imidazo[1,5-a]pyridin-6-yl)-2,4-difluorophenyl)-2-methylpyridine-3-sulfonamide

Into an 8 mL vial were added 3-[1-(4,5-dimethyl-1-[[2-(trimethylsilyl)ethoxy]methyl]imidazol-2-yl)imidazo[1,5-a]pyridin-6-yl]-2,4-difluoroaniline (150 mg, 0.32 mmol, 1 equiv), 5-chloro-2-methylpyridine-3-sulfonyl chloride (144 mg, 0.64 mmol, 2 equiv) and pyridine (3 mL). The resulting mixture was stirred for 2 h then concentrated. The residue was purified by silica gel column chromatography, eluting with PE/EtOAc (10:7) to afford 5-chloro-N-[3-[1-(4,5-dimethyl-1-[[2-(trimethylsilyl)ethoxy]methyl]imidazol-2-yl)imidazo[1,5-a]pyridin-6-yl]-2,4-difluorophenyl]-2-methylpyridine-3-sulfonamide (160 mg, 76% yield) as a light yellow solid.

LCMS (ES, m/z): [M+H]⁺: 659.

### Synthesis of Compound 146: 5-chloro-N-(3-(1-(4,5-dimethyl-1H-imidazol-2-yl)imidazo[1,5-a]pyridin-6-yl)-2,4-difluorophenyl)-2-methylpyridine-3-sulfonamide

Into an 8 mL vial were added 5-chloro-N-[3-[1-(4,5-dimethyl-1-[[2-(trimethylsilyl)ethoxy]methyl]imidazol-2-yl)imidazo[1,5-a]pyridin-6-yl]-2,4-difluorophenyl]-2-methylpyridine-3-sulfonamide (160 mg, 0.24 mmol, 1 equiv) and TFA (3 mL). The resulting mixture was stirred for 30 min at 40° C., then concentrated. The crude product was purified by Prep-HPLC with the following conditions: XBridge Prep C18 OBD Column, 5 μm, 19*150 mm; Mobile Phase: 24-41% MeCN/0.05% aqueous ammonia; Flow rate: 20 mL/min; to give 5-chloro-N-[3-[1-(4,5-dimethyl-1H-imidazol-2-yl)imidazo[1,5-a]pyridin-6-yl]-2,4-difluorophenyl]-2-methylpyridine-3-sulfonamide (34 mg, 26% yield) as a yellow solid.

LCMS (ES, m/z): [M+H]⁺: 529.

¹H NMR (300 MHz, DMSO-d₆) δ 12.02 (s, 1H), 8.62 (d, J=2.4 Hz, 1H), 8.50 (d, J=6.1 Hz, 2H), 8.17 (d, J=9.4 Hz, 1H), 8.07 (d, J=2.4 Hz, 1H), 7.32-7.18 (m, 1H), 7.05 (t, J=9.3 Hz, 1H), 6.87 (d, J=9.5 Hz, 1H), 2.77 (s, 3H), 2.15 (s, 6H).

### Example 162: Synthesis of 5-chloro-N-(2,4-difluoro-3-(1-(4,5,6,7-tetrahydro-1H-benzo[d]imidazol-2-yl) imidazo[1,5-a]pyridin-6-yl)phenyl)-2-methoxypyridine-3-sulfonamide (Compound 147)

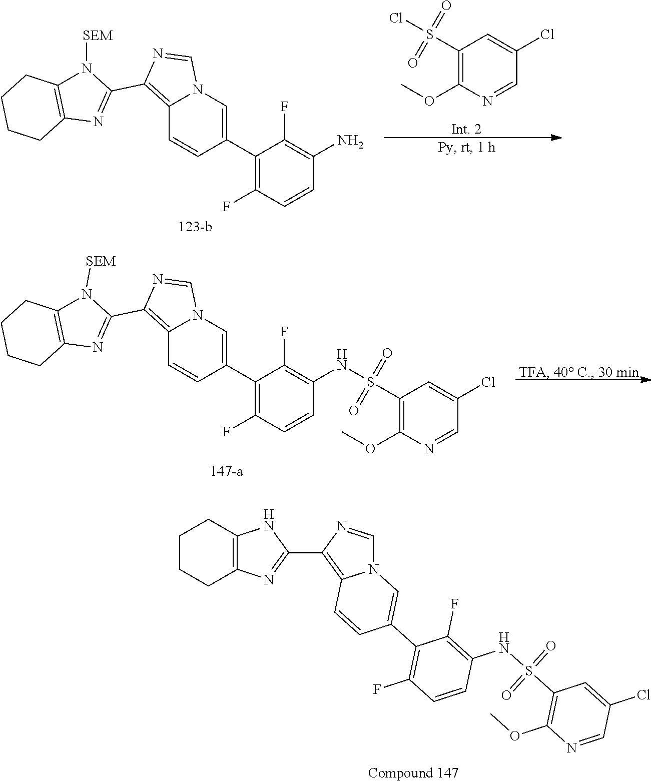 [SMILES: C[Si](C)(C)CCOCn1c(-c2ncn3cc(-c4c(F)ccc(N)c4F)ccc23)nc2c1CCCC2 ; COc1ncc(Cl)cc1S(=O)(=O)Cl ; COc1ncc(Cl)cc1S(=O)(=O)Nc1ccc(F)c(-c2ccc3c(-c4nc5c(n4COCC[Si](C)(C)C)CCCC5)ncn3c2)c1F ; COc1ncc(Cl)cc1S(=O)(=O)Nc1ccc(F)c(-c2ccc3c(-c4nc5c([nH]4)CCCC5)ncn3c2)c1F]

### Synthesis of 147-a: 5-chloro-N-(2,4-difluoro-3-(1-(1-((2-(trimethylsilyl)ethoxy)methyl)-4,5,6,7-tetrahydro-1H-benzo[d]imidazol-2-yl)imidazo[1,5-a]pyridin-6-yl)phenyl)-2-methoxypyridine-3-sulfonamide

Into an 8 mL vial were added 2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]aniline (150 mg, 0.3 mmol, 1 equiv), 5-chloro-2-methoxypyridine-3-sulfonyl chloride (146 mg, 0.6 mmol, 2 equiv) and pyridine (3 mL). The reaction mixture was stirred for 2 h then concentrated. The residue was purified by silica gel column chromatography, eluting with PE/EtOAc (10:7) to afford 5-chloro-N-[2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]phenyl]-2-methoxypyridine-3-sulfonamide (120 mg, 57% yield) as a light yellow solid.

LCMS (ES, m/z): [M+H]⁺: 701.

### Synthesis of Compound 147: 5-chloro-N-(2,4-difluoro-3-(1-(4,5,6,7-tetrahydro-1H-benzo[d]imidazol-2-yl) imidazo[1,5-a]pyridin-6-yl)phenyl)-2-methoxypyridine-3-sulfonamide

Into an 8 mL vial were added 5-chloro-N-[2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]phenyl]-2-methoxypyridine-3-sulfonamide (110 mg, 0.16 mmol, 1 equiv) and TFA (3 mL). The mixture was stirred for 30 min at 40° C. then concentrated. The crude product was purified by Prep-HPLC with the following conditions: XBridge Prep C18 OBD Column, 5 μm, 19*150 mm; Mobile Phase: 24-41% MeCN/0.05% aqueous ammonia; Flow rate: 20 mL/min; to give 5-chloro-N-(2,4-difluoro-3-(1-(4,5,6,7-tetrahydro-1H-benzo[d]imidazol-2-yl) imidazo[1,5-a]pyridin-6-yl)phenyl)-2-methoxypyridine-3-sulfonamide (37 mg, 41% yield) as a yellow solid.

LCMS (ES, m/z): [M+H]⁺: 571.

¹H NMR (300 MHz, DMSO-d₆) δ 11.38 (s, 1H), 8.45 (s, 2H), 8.37 (d, J=2.5 Hz, 1H), 8.18 (d, J=9.4 Hz, 1H), 8.03 (d, J=2.6 Hz, 1H), 7.35-7.21 (m, 1H), 7.06 (t, J=9.3 Hz, 1H), 6.80 (d, J=9.4 Hz, 1H), 3.87 (s, 3H), 2.57-2.51 (m, 4H), 1.79-1.73 (m, 4H).

### Example 163: Synthesis of N-(2,4-difluoro-3-(1-(4,5,6,7-tetrahydro-1H-benzo[d]imidazol-2-yl)imidazo[1,5-a]pyridin-6-yl)phenyl)-5-fluoro-2-methoxypyridine-3-sulfonamide (Compound 148)

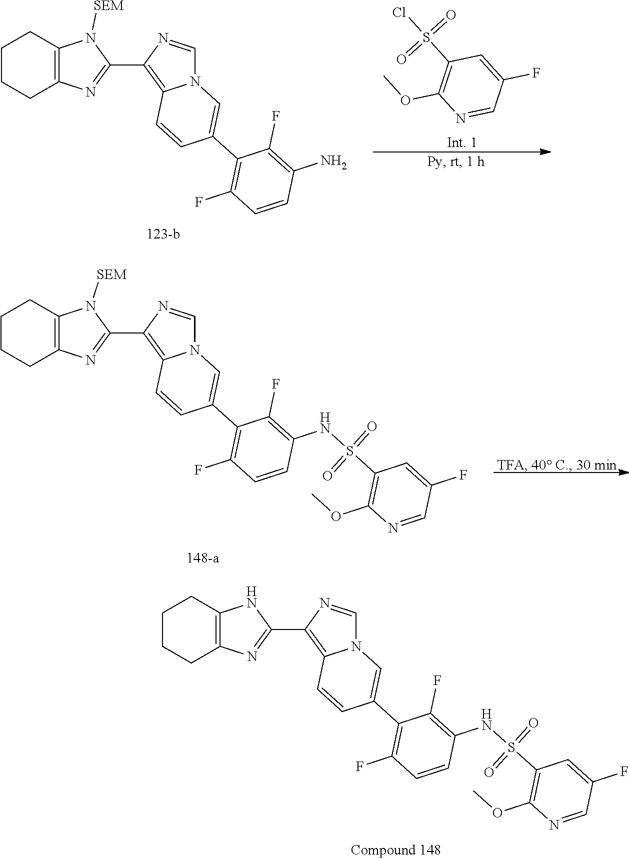 [SMILES: C[Si](C)(C)CCOCn1c(-c2ncn3cc(-c4c(F)ccc(N)c4F)ccc23)nc2c1CCCC2 ; COc1ncc(F)cc1S(=O)(=O)Cl ; COc1ncc(F)cc1S(=O)(=O)Nc1ccc(F)c(-c2ccc3c(-c4nc5c(n4COCC[Si](C)(C)C)CCCC5)ncn3c2)c1F ; COc1ncc(F)cc1S(=O)(=O)Nc1ccc(F)c(-c2ccc3c(-c4nc5c([nH]4)CCCC5)ncn3c2)c1F]

### Synthesis of 148-a: N-(2,4-difluoro-3-(1-(1-((2-(trimethylsilyl)ethoxy)methyl)-4,5,6,7-tetrahydro-1H-benzo[d]imidazol-2-yl)imidazo[1,5-a]pyridin-6-yl)phenyl)-5-fluoro-2-methoxypyridine-3-sulfonamide

Into an 8 mL vial were added 2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]aniline (150 mg, 0.3 mmol, 1 equiv), 5-fluoro-2-methoxypyridine-3-sulfonyl chloride (136 mg, 0.6 mmol, 2 equiv) and pyridine (3 mL). The resulting mixture was stirred for 2 h at room temperature then concentrated. The residue was purified by silica gel column chromatography, eluting with PE/EtOAc (10:7) to afford N-[2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]phenyl]-5-fluoro-2-methoxypyridine-3-sulfonamide (130 mg, 63% yield) as a light yellow solid.

LCMS (ES, m/z): [M+H]⁺: 685.

### Synthesis of Compound 148: N-(2,4-difluoro-3-(1-(4,5,6,7-tetrahydro-1H-benzo[d]imidazol-2-yl)imidazo[1,5-a]pyridin-6-yl)phenyl)-5-fluoro-2-methoxypyridine-3-sulfonamide

Into an 8 mL vial were added N-[2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]phenyl]-5-fluoro-2-methoxypyridine-3-sulfonamide (120 mg, 0.18 mmol, 1 equiv) and TFA (3 mL). The resulting mixture was stirred for 30 min at 40° C., then concentrated. The crude product was purified by Prep-HPLC with the following conditions: XBridge Prep C18 OBD Column, 5 μm, 19*150 mm; Mobile Phase: 24-41% MeCN/0.05% aqueous ammonia; Flow rate: 20 mL/min; to give N-[2,4-difluoro-3-[1-(4,5,6,7-tetrahydro-1H-1,3-benzodiazol-2-yl)imidazo[1,5-a]pyridin-6-yl]phenyl]-5-fluoro-2-methoxypyridine-3-sulfonamide (12 mg, 12% yield) as a yellow solid.

LCMS (ES, m/z): [M+H]⁺: 555.

¹H NMR (300 MHz, DMSO-d₆) δ 12.52-10.49 (m, 1H), 8.45 (s, 2H), 8.38-8.32 (m, 1H), 8.18 (d, J=9.5 Hz, 1H), 7.96 (dd, J=7.6, 3.0 Hz, 1H), 7.37-7.22 (m, 1H), 7.15-7.06 (m, 1H), 6.84-6.74 (m, 1H), 3.87 (s, 3H), 2.57-2.51 (m, 4H), 1.79-1.72 (m, 4H).

### Example 164: Synthesis of N-(2,4-difluoro-3-(1-(4,5,6,7-tetrahydro-1H-benzo[d]imidazol-2-yl) imidazo[1,5-a]pyridin-6-yl)phenyl)-5-fluoro-2-methylpyridine-3-sulfonamide (Compound 149)

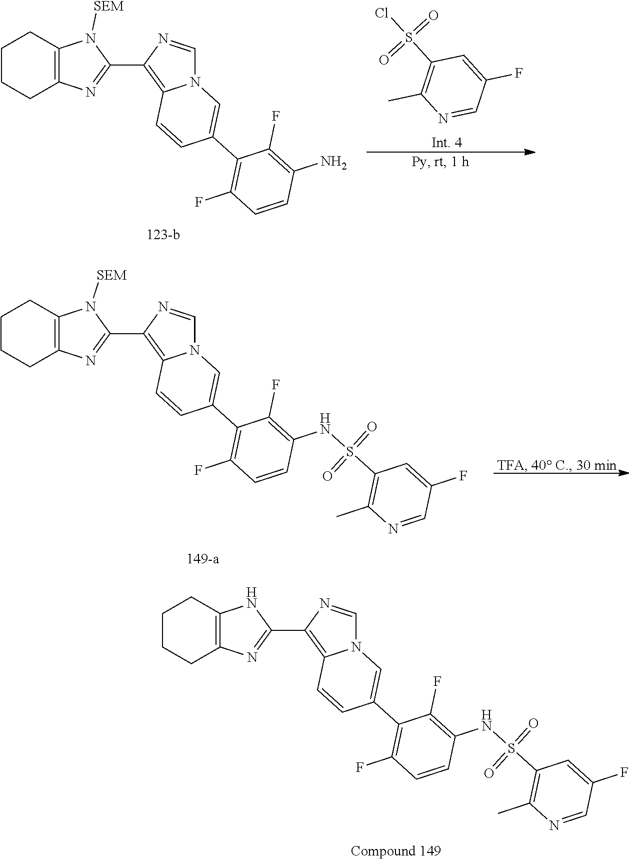 [SMILES: C[Si](C)(C)CCOCn1c(-c2ncn3cc(-c4c(F)ccc(N)c4F)ccc23)nc2c1CCCC2 ; Cc1ncc(F)cc1S(=O)(=O)Cl ; Cc1ncc(F)cc1S(=O)(=O)Nc1ccc(F)c(-c2ccc3c(-c4nc5c(n4COCC[Si](C)(C)C)CCCC5)ncn3c2)c1F ; Cc1ncc(F)cc1S(=O)(=O)Nc1ccc(F)c(-c2ccc3c(-c4nc5c([nH]4)CCCC5)ncn3c2)c1F]

### Synthesis of 149-a: N-(2,4-difluoro-3-(1-(1-((2-(trimethylsilyl)ethoxy)methyl)-4,5,6,7-tetrahydro-1H-benzo[d]imidazol-2-yl)imidazo[1,5-a]pyridin-6-yl)phenyl)-5-fluoro-2-methylpyridine-3-sulfonamide

Into an 8 mL vial were added 2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]aniline (150 mg, 0.3 mmol, 1 equiv), 5-fluoro-2-methylpyridine-3-sulfonyl chloride (136 mg, 0.6 mmol, 2 equiv) and pyridine (3 mL). The reaction was stirred for 2 h at room temperature then concentrated. The residue was purified by silica gel column chromatography, eluting with PE/EtOAc (10:7) to afford N-(2,4-difluoro-3-(1-(1-((2-(trimethylsilyl)ethoxy)methyl)-4,5,6,7-tetrahydro-1H-benzo[d]imidazol-2-yl)imidazo[1,5-a]pyridin-6-yl)phenyl)-5-fluoro-2-methylpyridine-3-sulfonamide (160 mg, 68% yield) as a light yellow solid.

LCMS (ES, m/z): [M+H]⁺: 669.

### Synthesis of Compound 159: N-(2,4-difluoro-3-(1-(4,5,6,7-tetrahydro-1H-benzo[d]imidazol-2-yl) imidazo[1,5-a]pyridin-6-yl)phenyl)-5-fluoro-2-methylpyridine-3-sulfonamide

Into an 8 mL vial were added N-[2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]phenyl]-5-fluoro-2-methylpyridine-3-sulfonamide (150 mg, 0.22 mmol, 1 equiv) and TFA (3 mL). The resulting mixture was stirred for 30 min at 40° C., then concentrated. The crude product was purified by Prep-HPLC with the following conditions: XBridge Prep C18 OBD Column, 5 μm, 19*150 mm; Mobile Phase: 24-41% MeCN/0.05% aqueous ammonia; Flow rate: 20 mL/min; to give N-[2,4-difluoro-3-[1-(4,5,6,7-tetrahydro-1H-1,3-benzodiazol-2-yl)imidazo[1,5-a]pyridin-6-yl]phenyl]-5-fluoro-2-methylpyridine-3-sulfonamide (47 mg, 39% yield) as a yellow solid.

LCMS (ES, m/z): [M+H]⁺: 539.

¹H NMR (300 MHz, DMSO-d₆) δ 11.84 (s, 1H), 8.62 (d, J=2.9 Hz, 1H), 8.52-8.45 (m, 2H), 8.18 (d, J=9.5 Hz, 1H), 7.93 (dd, J=8.3, 2.9 Hz, 1H), 7.35-7.21 (m, 1H), 7.16-7.03 (m, 1H), 6.83 (d, J=9.3 Hz, 1H), 2.77 (d, J=1.2 Hz, 3H), 2.59-2.53 (m, 4H), 1.80-1.74 (m, 4H).

### Example 165: Synthesis of 5-chloro-N-(2,4-difluoro-3-(1-(4,5,6,7-tetrahydro-1H-benzo[d]imidazol-2-yl)imidazo[1,5-a]pyridin-6-yl)phenyl)-2-methylpyridine-3-sulfonamide (Compound 150)

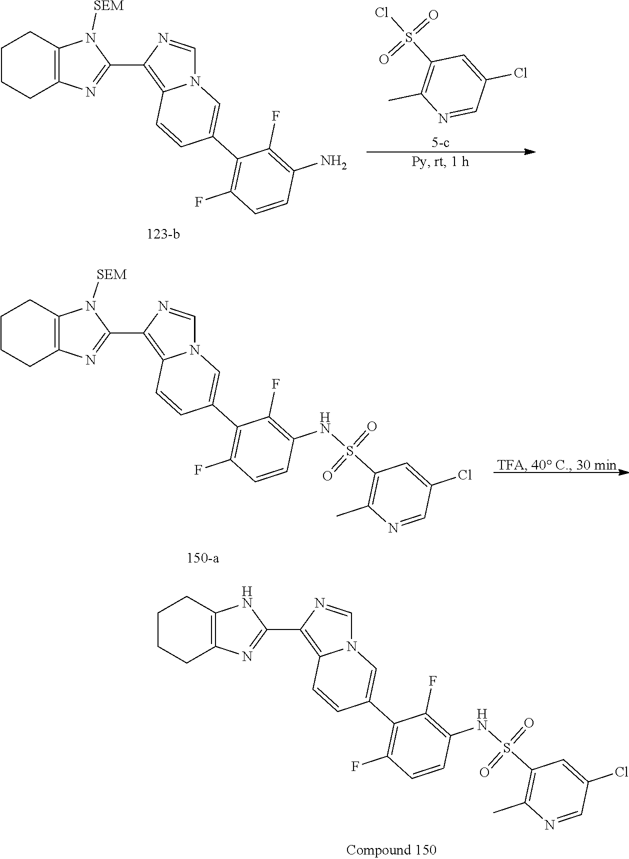 [SMILES: C[Si](C)(C)CCOCn1c(-c2ncn3cc(-c4c(F)ccc(N)c4F)ccc23)nc2c1CCCC2 ; Cc1ncc(Cl)cc1S(=O)(=O)Cl ; Cc1ncc(Cl)cc1S(=O)(=O)Nc1ccc(F)c(-c2ccc3c(-c4nc5c(n4COCC[Si](C)(C)C)CCCC5)ncn3c2)c1F ; Cc1ncc(Cl)cc1S(=O)(=O)Nc1ccc(F)c(-c2ccc3c(-c4nc5c([nH]4)CCCC5)ncn3c2)c1F]

### Synthesis of 150-a: 5-chloro-N-(2,4-difluoro-3-(1-(1-((2-(trimethylsilyl)ethoxy)methyl)-4,5,6,7-tetrahydro-1H-benzo[d]imidazol-2-yl)imidazo[1,5-a]pyridin-6-yl)phenyl)-2-methylpyridine-3-sulfonamide

Into an 8 mL vial were added 2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]aniline (150 mg, 0.3 mmol, 1 equiv), 5-chloro-2-methylpyridine-3-sulfonyl chloride (136 mg, 0.6 mmol, 2 equiv) and pyridine (3 mL). The resulting mixture was stirred for 2 h at room temperature then concentrated. The residue was purified by silica gel column chromatography, eluting with PE/EtOAc (10:7) to afford N-[2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]phenyl]-5-chloro-2-methylpyridine-3-sulfonamide (160 mg, 68% yield) as a light yellow solid.

LCMS (ES, m/z): [M+H]⁺: 685.

### Synthesis of Compound 150: 5-chloro-N-(2,4-difluoro-3-(1-(4,5,6,7-tetrahydro-1H-benzo[d]imidazol-2-yl)imidazo[1,5-a]pyridin-6-yl)phenyl)-2-methylpyridine-3-sulfonamide

Into an 8 mL vial were added 5-chloro-N-[2,4-difluoro-3-[1-(1-[[2-(trimethylsilyl)ethoxy]methyl]-4,5,6,7-tetrahydro-1,3-benzodiazol-2-yl)imidazo[1,5-a]pyridin-6-yl]phenyl]-2-methylpyridine-3-sulfonamide (140 mg, 0.2 mmol, 1 equiv) and TFA (3 mL). The resulting mixture was stirred for 30 min at 40° C. then concentrated. The crude product was purified by Prep-HPLC with the following conditions: XBridge Prep C18 OBD Column, 5 μm, 19*150 mm; Mobile Phase: 24-41% MeCN/0.05% aqueous ammonia; Flow rate: 20 mL/min; to give 5-chloro-N-[2,4-difluoro-3-[1-(4,5,6,7-tetrahydro-1H-1,3-benzodiazol-2-yl)imidazo[1,5-a]pyridin-6-yl]phenyl]-2-methylpyridine-3-sulfonamide (39 mg, 34% yield) as a yellow solid.

LCMS (ES, m/z): [M+H]⁺: 555.

¹H NMR (300 MHz, DMSO-d₆) δ 11.84 (s, 1H), 8.56 (d, J=2.5 Hz, 1H), 8.49-8.42 (m, 2H), 8.16 (d, J=9.4 Hz, 1H), 8.06 (d, J=2.5 Hz, 1H), 7.21 (td, J=9.2, 5.9 Hz, 1H), 7.03-6.91 (m, 1H), 6.88-6.78 (m, 1H), 2.77 (s, 3H), 2.58-2.52 (m, 4H), 1.80-1.72 (m, 4H).

### Example 166: Synthesis of N-[2-chloro-4-fluoro-3-[1-(1H-imidazol-2-yl)imidazo[1,5-a]pyrazin-6-yl]phenyl]-5-fluoro-2-methoxypyridine-3-sulfonamide (Compound 151)

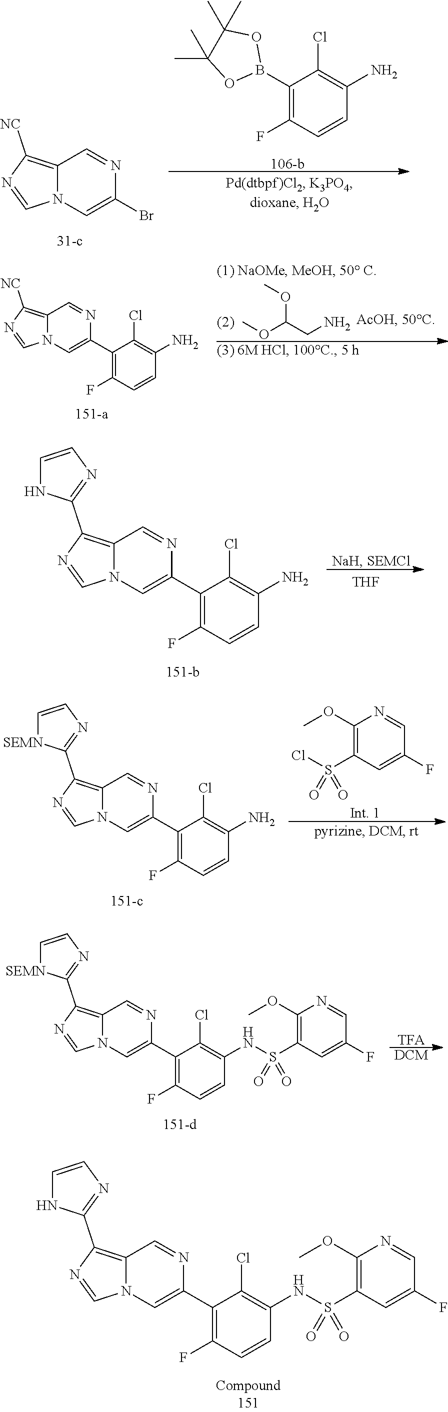

### Synthesis of 151-a: 6-(3-amino-2-chloro-6-fluorophenyl)imidazo[1,5-a]pyrazine-1-carbonitrile

To a stirred solution of 6-bromoimidazo[1,5-a]pyrazine-1-carbonitrile (350 mg, 1.57 mmol, 1 equiv) and 2-chloro-4-fluoro-3-(4,4,5,5-tetramethyl-1,3,2-dioxaborolan-2-yl)aniline (639 mg, 2.35 mmol, 1.5 equiv) in dioxane (4 mL) and H₂O (0.4 mL) were added Pd(dtbpf)Cl₂ (102 mg, 0.16 mmol, 0.1 equiv) and K₃PO₄ (999 mg, 4.7 mmol, 3 equiv) in portions at room temperature under nitrogen atmosphere. The resulting mixture was stirred for 5 h at 80° C., then cooled and quenched with H₂O (20 mL). After extraction with EA (3×10 mL), the combined organics were washed with brine (20 mL), dried over anhydrous Na₂SO₄ and concentrated. The residue was purified by prep-HPLC with the following conditions: Column, WelFlash™ C18-I, Spherical C18 20-40 m, 120 g; mobile phase: 10-55% MeCN/0.1% aqueous formic acid; to afford 6-(3-amino-2-chloro-6-fluorophenyl)imidazo[1,5-a]pyrazine-1-carbonitrile (190 mg, 42% yield) as a brown oil.

LCMS (ES, m/z): [M+H]⁺: 288

### Synthesis of 151-b: 2-chloro-4-fluoro-3-[1-(1H-imidazol-2-yl)imidazo[1,5-a]pyrazin-6-yl]aniline

To a stirred solution of 6-(3-amino-2-chloro-6-fluorophenyl)imidazo[1,5-a]pyrazine-1-carbonitrile (180 mg, 0.63 mmol, 1 equiv) in MeOH (4 mL) was added sodium methoxide (452 mg, 8.37 mmol, 13.4 equiv) in portions at 0° C. The reaction was stirred for 2 h at 50° C. The mixture was allowed to cool and 2,2-dimethoxyethanamine (66 mg, 0.63 mmol, 1.2 equiv) was added, followed by AcOH (0.15 mL, 2.51 mmol, 5 equiv) dropwise. The resulting mixture was stirred for 4 h at 50° C., then cooled. 6 M aqueous HCl (2.7 mL) was added, then the mixture heated for 5 h at 100° C. After cooling, H₂O (10 mL) was added, and the mixture basified to pH 8 with 30% aqueous NaOH. Following extraction with EA (3×5 mL), the combined organics were washed with brine (10 mL), dried over anhydrous Na₂SO₄ and concentrated. The residue was purified by prep-HPLC with the following conditions: Column, WelFlash™ C18-I, Spherical C18 20-40 m, 120 g; mobile phase: 30-70% MeCN/0.1% aqueous formic acid; to afford 2-chloro-4-fluoro-3-[1-(1H-imidazol-2-yl)imidazo[1,5-a]pyrazin-6-yl]aniline (160 mg, 92% yield) as a brown oil.

LCMS (ES, m/z): [M+H]⁺: 329

### Synthesis of 151-c: 2-chloro-4-fluoro-3-[1-(1-[[2-(trimethylsilyl)ethoxy]methyl]imidazol-2-yl)imidazo[1,5-a]pyrazin-6-yl]aniline

To a stirred solution of 2-chloro-4-fluoro-3-[1-(1H-imidazol-2-yl)imidazo[1,5-a]pyrazin-6-yl]aniline (180 mg, 0.54 mmol, 1 equiv) in THE (4 mL) was added NaH (46 mg, 1.9 mmol, 3.5 equiv) in portions at 0° C. The resulting mixture was stirred for 0.5 h at 0° C., then SEMCI (137 mg, 0.82 mmol, 1.5 equiv) was added dropwise over 5 min at 0° C. The reaction was stirred for 1 h at room temperature, then quenched by the addition of H₂O (5 mL). The resulting mixture was extracted with EA (3×5 mL), and the combined organics were washed with brine (10 mL), dried over anhydrous Na₂SO₄ and concentrated. The residue was purified by prep-HPLC with the following conditions: Column, WelFlash™ C18-I, Spherical C18 20-40 m, 120 g; mobile phase; 30-80% MeCN/0.1% aqueous formic acid; to afford 2-chloro-4-fluoro-3-[1-(1-[[2-(trimethylsilyl)ethoxy]methyl]imidazol-2-yl)imidazo[1,5-a]pyrazin-6-yl]aniline (242 mg, 96% yield) as a yellow solid.

LCMS (ES, m/z): [M+H]⁺: 459

### Synthesis of 151-d: N-[2-chloro-4-fluoro-3-[1-(1-[[2-(trimethylsilyl)ethoxy]methyl]imidazol-2-yl)imidazo[1,5-a]pyrazin-6-yl]phenyl-5-fluoro-2-methoxypyridine-3-sulfonamide

To a stirred solution of 2-chloro-4-fluoro-3-[1-(1-[[2-(trimethylsilyl)ethoxy]methyl]imidazol-2-yl)imidazo[1,5-a]pyrazin-6-yl]aniline (247 mg, 0.54 mmol, 1 equiv) in pyridine (2 mL) was added 5-fluoro-2-methoxypyridine-3-sulfonyl chloride (182 mg, 0.81 mmol, 1.5 equiv) in DCM (0.5 mL). The reaction was stirred for 1 h at room temperature. The resulting mixture was concentrated, and the residue purified by prep-HPLC with the following conditions: Column, WelFlash™ C18-I, Spherical C18 20-m, 120 g; mobile phase: 30-80% MeCN/0.1% aqueous formic acid; to afford N-[2-chloro-4-fluoro-3-[1-(1-[[2-(trimethylsilyl)ethoxy]methyl]imidazol-2-yl)imidazo[1,5-a]pyrazin-6-yl]phenyl]-5-fluoro-2-methoxypyridine-3-sulfonamide (135 mg, 39% yield) as a yellow oil.

LCMS (ES, m/z): [M+H]⁺: 648

### Synthesis of Compound 151: N-[2-chloro-4-fluoro-3-[1-(1H-imidazol-2-yl)imidazo[1,5-a]pyrazin-6-yl]phenyl]-5-fluoro-2-methoxypyridine-3-sulfonamide

Into a 40 mL vial was placed N-[2-chloro-4-fluoro-3-[1-(1-[[2-(trimethylsilyl)ethoxy]methyl]imidazol-2-yl)imidazo[1,5-a]pyrazin-6-yl]phenyl]-5-fluoro-2-methoxypyridine-3-sulfonamide (140 mg, 0.22 mmol, 1 equiv), DCM (3 mL) and TFA (1 mL). The resulting solution was stirred for 2 h at room temperature, then concentrated. The residue was purified by prep-HPLC with the following conditions: Column, welch Vltimate XB-C18, 50×250 mm, 10 μm; Mobile Phase: 30-75% MeCN/0.1% aqueous formic acid; to give N-[2-chloro-4-fluoro-3-[1-(1H-imidazol-2-yl)imidazo[1,5-a]pyrazin-6-yl]phenyl]-5-fluoro-2-methoxypyridine-3-sulfonamide (50 mg, 45% yield) as a yellow solid.

LCMS (ES, m/z): [M+H]⁺: 518

¹H NMR (300 MHz, DMSO-d₆) δ 12.71 (s, 1H), 10.41 (s, 1H), 9.56 (d, J=2.4 Hz, 1H), 8.64 (d, J=0.6 Hz, 1H), 8.53 (d, J=1.7 Hz, 1H), 8.46 (d, J=3.0 Hz, 1H), 8.01 (dd, J=7.3, 3.0 Hz, 1H), 7.49 (dd, J=8.9, 5.7 Hz, 1H), 7.40 (t, J=8.8 Hz, 1H), 7.17 (s, 2H), 3.88 (s, 3H).

### Example 167: Synthesis of (6R)-6-[2-chloro-6-fluoro-3-(5-fluoro-2-methoxypyridine-3-sulfonamido)phenyl]-N-methyl-5H,6H,7H,8H-imidazo[1,5-a]pyridine-1-carboxamide (first eluting isomer, 35 me, stereochemistry randomly assigned) and (6S)-6-[2-chloro-6-fluoro-3-(5-fluoro-2-methoxypyridine-3-sulfonamido)phenyl]-N-methyl-5H,6H,7H,8H-imidazo[1,5-a]pyridine-1-carboxamide (Compounds 152-1 & 152-2)

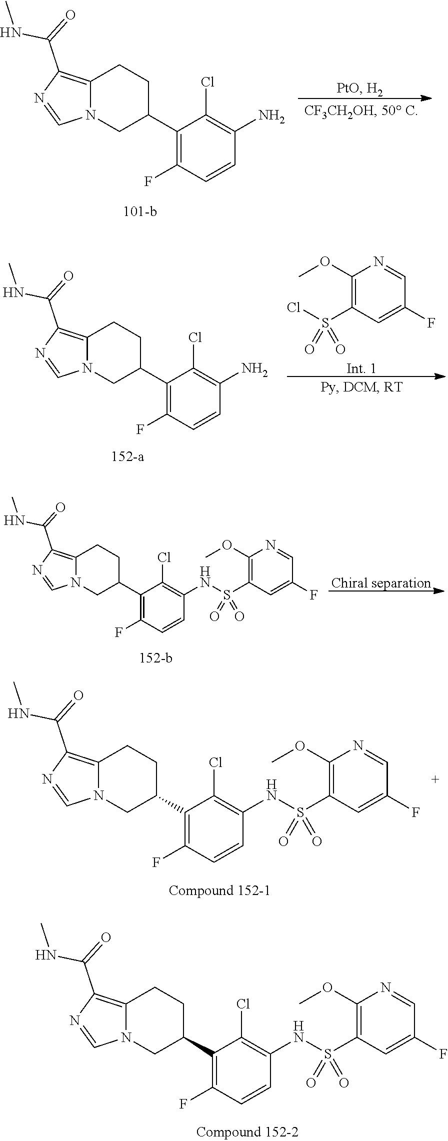 [SMILES: CNC(=O)c1ncn2c1CCC(c1c(F)ccc(N)c1Cl)C2 ; CNC(=O)c1ncn2c1CCC(c1c(F)ccc(N)c1Cl)C2 ; COc1ncc(F)cc1S(=O)(=O)Cl ; CNC(=O)c1ncn2c1CCC(c1c(F)ccc(NS(=O)(=O)c3cc(F)cnc3OC)c1Cl)C2 ; CNC(=O)c1ncn2c1CC[C@H](c1c(F)ccc(NS(=O)(=O)c3cc(F)cnc3OC)c1Cl)C2 ; CNC(=O)c1ncn2c1CC[C@@H](c1c(F)ccc(NS(=O)(=O)c3cc(F)cnc3OC)c1Cl)C2]

### Synthesis of 152-a: 6-(3-amino-2-chloro-6-fluorophenyl)-N-methyl-5,6,7,8-imidazo[1,5-a]pyridine-1-carboxamide

To a solution of 6-(3-amino-2-chloro-6-fluorophenyl)-N-methylimidazo[1,5-a]pyridine-1-carboxamide (400 mg, 1.2 mmol, 1 equiv) in 10 mL MeOH and 2M aqueous HCl (1 mL) was added PtO₂ (28 mg) in a pressure tank. The mixture was hydrogenated at 50° C. under 10 atm of hydrogen pressure for 2 hr, then filtered through a Celite pad and concentrated. The residue was purified by silica gel column chromatography, eluting with PE:EA (1:1) to afford 6-(3-amino-2-chloro-6-fluorophenyl)-N-methyl-5,6,7,8-imidazo[1,5-a]pyridine-1-carboxamide (120 mg, 30% yield) as a yellow solid.

LCMS (ES, m/z): [M+H]⁺: 323

### Synthesis of Compounds 152-1 & 152-2: (6R)-6-[2-chloro-6-fluoro-3-(5-fluoro-2-methoxypyridine-3-sulfonamido)phenyl]-N-methyl-5H,6H,7H,8H-imidazo[1,5-a]pyridine-1-carboxamide (first eluting isomer, 35 mg, stereochemistry randomly assigned) and (6S)-6-[2-chloro-6-fluoro-3-(5-fluoro-2-methoxypyridine-3-sulfonamido)phenyl]-N-methyl-5H,6H,7H,8H-imidazo[1,5-a]pyridine-1-carboxamide

To a solution of 6-(3-amino-2-chloro-6-fluorophenyl)-N-methyl-5H,6H,7H,8H-imidazo[1,5-a]pyridine-1-carboxamide (120 mg, 0.3 mmol, 1 equiv) and pyridine (88 mg, 1 mmol, 3 equiv) in DCM (5 mL) was added 5-fluoro-2-methoxypyridine-3-sulfonyl chloride (125 mg, 0.5 mmol, 1.5 equiv). The reaction mixture was stirred for 1 hr, then concentrated. The residue was purified by column chromatography over silica gel (eluent: PE:EA=2:1) to afford 6-[2-chloro-6-fluoro-3-(5-fluoro-2-methoxypyridine-3-sulfonamido)phenyl]-N-methyl-5,6,7,8-imidazo[1,5-a]pyridine-1-carboxamide (80 mg, 42% yield) as a white solid. The racemic mixture of 6-[2-chloro-6-fluoro-3-(5-fluoro-2-methoxypyridine-3-sulfonamido)phenyl]-N-methyl-5H,6H,7H,8H-imidazo[1,5-a]pyridine-1-carboxamide (100 mg) was separated by chiral HPLC using the following conditions: Column, CHIRALAPLC, IC, 250×20 mm, 5 μm; Mobile Phase: 50% EtOH/Hexane; to give (6R)-6-[2-chloro-6-fluoro-3-(5-fluoro-2-methoxypyridine-3-sulfonamido)phenyl]-N-methyl-5H,6H,7H,8H-imidazo[1,5-a]pyridine-1-carboxamide (first eluting isomer, 35 mg, stereochemistry randomly assigned) and (6S)-6-[2-chloro-6-fluoro-3-(5-fluoro-2-methoxypyridine-3-sulfonamido)phenyl]-N-methyl-5H,6H,7H,8H-imidazo[1,5-a]pyridine-1-carboxamide (second eluting isomer, 33 mg, stereochemistry assigned as opposite to other isomer) as white solids.

First Eluting Isomer

LCMS (ES, m/z): [M+H]⁺: 512

¹H NMR (300 MHz, DMSO-d₆) δ 10.30 (s, 1H), 8.48 (d, J=3.0 Hz, 1H), 7.99 (dd, J=7.3, 3.0 Hz, 1H), 7.75 (d, J=4.9 Hz, 1H), 7.56 (s, 1H), 7.35-7.21 (m, 2H), 4.26 (dd, J=12.5, 5.4 Hz, 1H), 4.12 (t, J=11.9 Hz, 1H), 3.85 (s, 3H), 3.69 (s, 1H), 3.39 (d, J=3.0 Hz, 1H), 2.86 (ddd, J=17.6, 11.7, 5.9 Hz, 1H), 2.71 (d, J=4.8 Hz, 3H), 2.25 (d, J=15.2 Hz, 1H), 1.98 (d, J=12.7 Hz, 1H).

Second Eluting Isomer

LCMS (ES, m/z): [M+H]⁺: 512

¹H NMR (300 MHz, DMSO-d₆) δ 10.30 (s, 1H), 8.47 (d, J=3.0 Hz, 1H), 7.99 (dd, J=7.3, 3.0 Hz, 1H), 7.75 (q, J=4.7 Hz, 1H), 7.56 (s, 1H), 7.38-7.17 (m, 2H), 4.26 (dd, J=12.4, 5.4 Hz, 1H), 4.12 (t, J=11.9 Hz, 1H), 3.85 (s, 3H), 3.69 (s, 1H), 3.39 (d, J=3.0 Hz, 1H), 2.86 (ddd, J=17.7, 11.8, 6.1 Hz, 1H), 2.71 (d, J=4.8 Hz, 3H), 2.24 (d, J=13.7 Hz, 1H), 1.98 (d, J=13.2 Hz, 1H).

### Example 168: Synthesis of methyl 2-[6-[2,6-difluoro-3-(5-fluoro-2-methoxypyridine-3-sulfonamido)phenyl]imidazo[1,5-a]pyrazin-1-yl]-3H-1,3-benzodiazole-5-carboxylate (Compound 153)

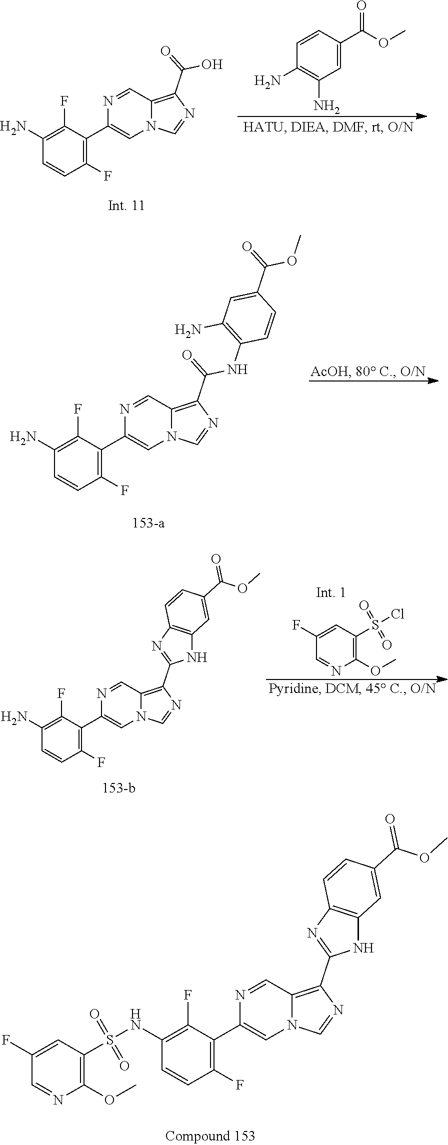 [SMILES: Nc1ccc(F)c(-c2cn3cnc(C(=O)O)c3cn2)c1F ; COC(=O)c1ccc(N)c(N)c1 ; COC(=O)c1ccc(NC(=O)c2ncn3cc(-c4c(F)ccc(N)c4F)ncc23)c(N)c1 ; COC(=O)c1ccc2nc(-c3ncn4cc(-c5c(F)ccc(N)c5F)ncc34)[nH]c2c1 ; COc1ncc(F)cc1S(=O)(=O)Cl ; COC(=O)c1ccc2nc(-c3ncn4cc(-c5c(F)ccc(NS(=O)(=O)c6cc(F)cnc6OC)c5F)ncc34)[nH]c2c1]

### Synthesis of 153-a: methyl 3-amino-4-[6-(3-amino-2,6-difluorophenyl)imidazo[1,5-a]pyrazine-1-amido]benzoate

Into a 250 mL 3-necked round-bottom flask was placed 6-(3-amino-2,6-difluorophenyl)imidazo[1,5-a]pyrazine-1-carboxylic acid (2 g, 6.9 mmol, 1 equiv), methyl 3,4-diaminobenzoate (1.15 g, 6.9 mmol, 1 equiv), HATU (3.93 g, 10 mmol, 1.5 equiv), DIEA (1.8 g, 13.8 mmol, 2 equiv) and DMF (100 mL). The resulting solution was stirred for 2 h then quenched by the addition of water (150 mL). The solid formed was collected by filtration and dried to give methyl 3-amino-4-[6-(3-amino-2,6-difluorophenyl)imidazo[1,5-a]pyrazine-1-amido]benzoate (2.3 g, 69% yield) as a yellow solid.

LCMS (ES, m/z): [M+H]⁺: 439.

### Synthesis of 153-b: methyl 2-[6-(3-amino-2,6-difluorophenyl)imidazo[1,5-a]pyrazin-1-yl]-3H-1,3-benzodiazole-5-carboxylate

Into a 50 mL 3-necked round-bottom flask was placed methyl 3-amino-4-[6-(3-amino-2,6-difluorophenyl)imidazo[1,5-a]pyrazine-1-amido]benzoate (2.3 g, 5.3 mmol, 1 equiv) in AcOH (15 mL). The resulting solution was stirred for 12 h at 80° C. then concentrated. The crude product was purified by Flash-Prep-HPLC with the following conditions: sunfire Prep C18 OBD Column, 50*250 mm 5 μm 10 nm; Mobile Phase: 10-45% MeCN/0.1% aqueous formic acid; to give methyl-2-[6-(3-amino-2,6-difluorophenyl)imidazo[1,5-a]pyrazin-1-yl]-3H-1,3-benzodiazole-5-carboxylate (1.0 g, 43% yield) as a yellow solid.

LCMS (ES, m/z): [M+H]⁺: 421

¹H NMR (300 MHz, DMSO-d₆) δ 13.43 (s, 1H), 9.82 (d, J=1.6 Hz, 1H), 8.83 (s, 1H), 8.73 (s, 1H), 8.23 (d, J=47.0 Hz, 1H), 7.74 (d, J=71.1 Hz, 2H), 7.04-6.82 (m, 2H), 5.18 (s, 2H), 3.90 (s, 3H).

### Synthesis of Compound 153: methyl 2-[6-[2,6-difluoro-3-(5-fluoro-2-methoxypyridine-3-sulfonamido)phenyl]imidazo[1,5-a]pyrazin-1-yl]-3H-1,3-benzodiazole-5-carboxylate

Into a 25 mL 3-necked round-bottom flask was placed methyl 2-[6-(3-amino-2,6-difluorophenyl)imidazo[1,5-a]pyrazin-1-yl]-3H-1,3-benzodiazole-5-carboxylate (100 mg, 0.24 mmol, 1 equiv), DCM (5 mL), pyridine (0.5 mL) and 5-fluoro-2-methoxypyridine-3-sulfonyl chloride (107 mg, 0.48 mmol, 2 equiv). The resulting solution was stirred for 12 h at 45° C. then concentrated. The crude product was purified by Flash-Prep-HPLC with the following conditions: sunfire Prep C18 OBD Column, 50*250 mm 5 μm 10 nm, Mobile Phase 25-65% MeCN/0.1% aqueous formic acid; to give methyl 2-[6-[2,6-difluoro-3-(5-fluoro-2-methoxypyridine-3-sulfonamido)phenyl]imidazo[1,5-a]pyrazin-1-yl]-3H-1,3-benzodiazole-5-carboxylate (55 mg, 37% yield) as a yellow solid.

LCMS (ES, m/z): [M+H]⁺: 610

¹H NMR (300 MHz, DMSO-d₆) δ 13.44 (s, 1H), 10.46 (s, 1H), 9.79 (d, J=1.6 Hz, 1H), 8.84 (s, 1H), 8.73 (d, J=1.6 Hz, 1H), 8.48 (d, J=3.0 Hz, 1H), 8.19 (s, 1H), 8.05 (dd, J=7.3, 3.0 Hz, 1H), 7.87 (d, J=8.2 Hz, 1H), 7.44 (td, J=8.8, 5.8 Hz, 1H), 7.33-7.21 (m, 1H), 3.91 (s, 3H), 3.90 (s, 3H).

### Example 169: Synthesis of (6S)-6-[3-(5-chloro-2-methoxypyridine-3-sulfonamido)-2,6-difluorophenyl]-N-methyl-5H,6H,8H-imidazo[4,3-cl[1,4]oxazine-1-carboxamide and (6R)-6-[13-(5-chloro-2-methoxypyridine-3-sulfonamido)-2,6-difluorophenyl]-N-methyl-5H,6H,8H-imidazo[4,3-cl[1,4]oxazine-1-carboxamide hydrochloride (Compounds 154-1 & 154-2)

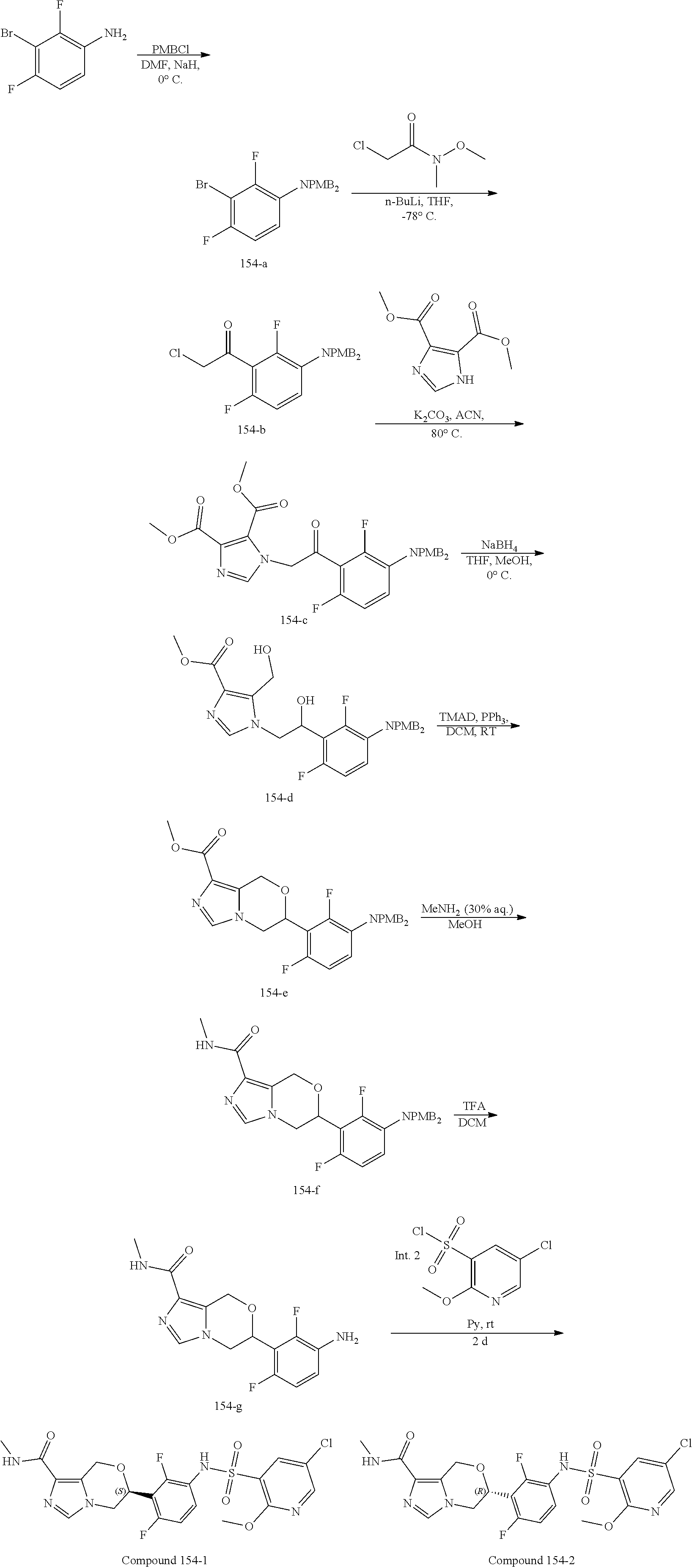

### Synthesis of 154-a: 3-bromo-2,4-difluoro-N,N-bis[(4-methoxyphenyl)methyl]aniline

Into a 500 mL 3-necked round-bottom flask purged and maintained with an inert atmosphere of nitrogen, cooled in an ice bath, was placed 3-bromo-2,4-difluoroaniline (10 g, 48 mmol, 1 equiv), DMF (200 mL), 60% NaH (3.46 g, 144 mmol, 3 equiv) and PMBCl (22.6 g, 144 mmol, 3 equiv). The resulting solution was stirred for 5 h at 0° C. The reaction was quenched by the addition of water (300 mL) and extracted with ethyl acetate (2×200 mL). The combined organics were washed with 200 ml of water and 200 mL of brine, dried over anhydrous sodium sulfate and concentrated. The residue was applied to a silica gel column, eluting with ethyl acetate/petroleum ether (1:3) to give 3-bromo-2,4-difluoro-N,N-bis[(4-methoxyphenyl)methyl]aniline (15.6 g, 72% yield) as a light yellow oil.

LCMS (ES, m/z): [M+H]⁺: 448

### Synthesis of 154-b: 1-(3-[bis[(4-methoxyphenyl)methyl]amino]-2,6-difluorophenyl)-2-chloroethanone

Into a 500 mL 3-necked round-bottom flask, purged and maintained with an inert atmosphere of nitrogen, was placed 3-bromo-2,4-difluoro-N,N-bis[(4-methoxyphenyl)methyl]aniline (15.6 g, 34.8 mmol, 1 equiv) in THE (300 mL). 2.5 M n-BuLi in hexanes (17 mL, 42.5 mmol, 1.2 equiv) was added dropwise with stirring at −78° C., and the reaction stirred for 1 h at low temperature. To the resultant mixture was added a solution of 2-chloro-N-methoxy-N-methylacetamide (6.7 g, 48.7 mmol, 1.4 equiv) in THE (6 mL) dropwise with stirring at −78° C. This mixture was allowed to warm to room temperature over 5 h. The reaction was quenched by the addition of water (300 mL), then extracted with ethyl acetate (3×200 mL). The combined organics were washed with 300 ml of water and 300 mL of brine, dried over anhydrous sodium sulfate and concentrated. The residue was applied to a silica gel column, eluting with ethyl acetate/petroleum ether (1:2) to give 1-(3-[bis[(4-methoxyphenyl)methyl]amino]-2,6-difluorophenyl)-2-chloroethanone (7 g, 45% yield) as a light yellow solid.

LCMS (ES, m/z): [M+H]⁺: 446

### Synthesis of 154-c: 4,5-dimethyl 1-[2-(3-[bis[(4-methoxyphenyl)methyl]amino]-2,6-difluorophenyl)-2-oxoethyl]imidazole-4,5-dicarboxylate

Into a 250 mL 3-necked round-bottom flask was placed 1-(3-[bis[(4-methoxyphenyl)methyl]amino]-2,6-difluorophenyl)-2-chloroethanone (4 g, 8.9 mmol, 1 equiv), MeCN (100 mL), 4,5-dimethyl 1H-imidazole-4,5-dicarboxylate (1.8 g, 9.9 mmol, 1.1 equiv) and K₂CO₃ (2.5 g, 17.9 mmol, 2 equiv). The resulting solution was stirred overnight at 80° C. then cooled and filtered. The filtrate was concentrated and applied to a silica gel column, eluting with ethyl acetate/petroleum ether (1:2). 4,5-Dimethyl 1-[2-(3-[bis[(4-methoxyphenyl)methyl]amino]-2,6-difluorophenyl)-2-oxoethyl]imidazole-4,5-dicarboxylate (3 g, 56% yield) was isolated as a white solid.

LCMS (ES, m/z): [M+H]⁺: 594

### Synthesis of 154-d: methyl 1-[2-(3-[bis[(4-methoxyphenyl)methyl]amino]-2,6-difluorophenyl)-2-hydroxyethyl]-5-(hydroxymethyl)imidazole-4-carboxylate

Into a 250 mL 3-necked round-bottom flask was placed 4,5-dimethyl 1-[2-(3-[bis[(4-methoxyphenyl)methyl]amino]-2,6-difluorophenyl)-2-oxoethyl]imidazole-4,5-dicarboxylate (3 g, 5.1 mmol, 1 equiv), THE (60 mL), NaBH₄ (0.57 g, 15.2 mmol, 3 equiv) and MeOH (10 mL). The resulting solution was stirred for 5 h then concentrated. The residue was applied to a silica gel column, eluting with ethyl acetate/petroleum ether (1:1). Methyl 1-[2-(3-[bis[(4-methoxyphenyl)methyl]amino]-2,6-difluorophenyl)-2-hydroxyethyl]-5-(hydroxymethyl)imidazole-4-carboxylate (2 g, 70% yield) was isolated as a white solid.

LCMS (ES, m/z): [M+H]⁺: 568

### Synthesis of 154-e: methyl 6-(3-[bis[(4-methoxyphenyl)methyl]amino]-2,6-difluorophenyl)-5H,6H,8H-imidazo[4,3-c][1,4]oxazine-1-carboxylate

Into a 100 mL round-bottom flask was placed methyl 1-[2-(3-[bis[(4-methoxyphenyl)methyl]amino]-2,6-difluorophenyl)-2-hydroxyethyl]-5-(hydroxymethyl)imidazole-4-carboxylate (1 g, 1.8 mmol, 1 equiv), DCM (50 mL), TMAD (0.46 g, 2.7 mmol, 1.5 equiv) and PPh₃ (0.69 g, 2.6 mmol, 1.5 equiv). The resulting solution was stirred overnight, then concentrated. The residue was applied to a silica gel column, eluting with ethyl acetate/petroleum ether (1:2) to give methyl 6-(3-[bis[(4-methoxyphenyl)methyl]amino]-2,6-difluorophenyl)-5H,6H,8H-imidazo[4,3-c][1,4]oxazine-1-carboxylate (160 mg, 17% yield) as a white solid.

LCMS (ES, m/z): [M+H]⁺: 550

### Synthesis of 154-f: 6-(3-[bis[(4-methoxyphenyl)methyl]amino]-2,6-difluorophenyl)-N-methyl-5H,6H,8H-imidazo[4,3-c][1,4]oxazine-1-carboxamide

To a stirred solution of methyl 6-(3-[bis[(4-methoxyphenyl)methyl]amino]-2,6-difluorophenyl)-5H,6H,8H-imidazo[4,3-c][1,4]oxazine-1-carboxylate (160 mg, 0.29 mmol, 1 equiv) in MeOH (5 mL) was added 33% aqueous methylamine solution (5 mL). The reaction was stirred for 24 h, then concentrated. The residue was purified by prep-HPLC with the following conditions: Column, WelFlash™ C18-I, Spherical C18 20-40 μm, 120 g; mobile phase: 25-95% MeCN/0.1% aqueous formic acid; to give 6-(3-[bis[(4-methoxyphenyl)methyl]amino]-2,6-difluorophenyl)-N-methyl-5H,6H,8H-imidazo[4,3-c][1,4]oxazine-1-carboxamide (140 mg, 88% yield) as a white solid.

LCMS (ES, m/z): [M+H]⁺: 549.

### Synthesis of 154-g: 6-(3-amino-2,6-difluorophenyl)-N-methyl-5H,6H,8H-imidazo[4,3-c][1,4]oxazine-1-carboxamide

To a stirred solution of 6-(3-[bis[(4-methoxyphenyl)methyl]amino]-2,6-difluorophenyl)-N-methyl-5H,6H,8H-imidazo[4,3-c][1,4]oxazine-1-carboxamide (135 mg, 0.25 mmol, 1 equiv) in DCM (3 mL) was added TFA (1 mL) dropwise at 0° C. The resulting mixture was stirred for 6 h at room temperature then was concentrated. The residue was purified by prep-HPLC with the following conditions: Column, WelFlash™ C18-I, Spherical C18 20-40 μm, 120 g; mobile phase: 20-70% MeCN/0.1% aqueous formic acid; to give 6-(3-amino-2,6-difluorophenyl)-N-methyl-5H,6H,8H-imidazo[4,3-c][1,4]oxazine-1-carboxamide (60 mg, 79% yield) as a white solid.

LCMS (ES, m/z): [M+H]⁺: 309.

### Synthesis of Compounds 154-1 & 154-2: (6S)-6-[3-(5-chloro-2-methoxypyridine-3-sulfonamido)-2,6-difluorophenyl]-N-methyl-5H,6H,8H-imidazo[4,3-c][1,4]oxazine-1-carboxamide and (6R)-6-[3-(5-chloro-2-methoxypyridine-3-sulfonamido)-2,6-difluorophenyl]-N-methyl-5H,6H,8H-imidazo[4,3-c][1,4]oxazine-1-carboxamide hydrochloride

To a stirred solution of 6-(3-amino-2,6-difluorophenyl)-N-methyl-5H,6H,8H-imidazo[4,3-c][1,4]oxazine-1-carboxamide (55 mg, 0.18 mmol, 1 equiv) in pyridine (2 mL) was added 5-chloro-2-methoxypyridine-3-sulfonyl chloride (130 mg, 0.54 mmol, 3 equiv) in portions at room temperature. The resulting mixture was stirred for 2 days then concentrated. The residue was purified by prep-HPLC with the following conditions: Column, welch Vltimate XB-C18, 50×250 mm, 10 μm; Mobile Phase: 20-50% MeCN/0.1% aqueous formic acid to give the racemic product. The enantiomers were separated by Chiral-Prep-HPLC with the following conditions: Column, CHIRALPAK IC, 20*250 mm, 5 m; Mobile Phase: 30% EtOH/Hexane (containing 0.1% DEA); to give (6S)-6-[3-(5-chloro-2-methoxypyridine-3-sulfonamido)-2,6-difluorophenyl]-N-methyl-5H,6H,8H-imidazo[4,3-c][1,4]oxazine-1-carboxamide (first eluting isomer, 3.4 mg, 4% yield, stereochemistry randomly assigned) and (6R)-6-[3-(5-chloro-2-methoxypyridine-3-sulfonamido)-2,6-difluorophenyl]-N-methyl-5H,6H,8H-imidazo[4,3-c][1,4]oxazine-1-carboxamide (second eluting isomer, 4.1 mg, 4% yield, stereochemistry assigned as opposite to other isomer) as white solids.

LCMS (ES, m/z): [M+H]⁺: 514 for both isomers

¹H NMR (300 MHz, DMSO-d₆) δ 10.39 (s, 1H), 8.50 (d, J=2.6 Hz, 1H), 8.05 (d, J=2.6 Hz, 1H), 7.87 (d, J=4.9 Hz, 1H), 7.68 (s, 1H), 7.42-7.28 (m, 1H), 7.12 (t, J=9.5 Hz, 1H), 5.29-5.13 (m, 2H), 5.00 (d, J=15.9 Hz, 1H), 4.35 (dd, J=12.6, 3.6 Hz, 1H), 4.19 (t, J=11.7 Hz, 1H), 3.91 (s, 3H), 2.72 (d, J=4.8 Hz, 3H).

¹H NMR (300 MHz, DMSO-d₆) δ 10.39 (s, 1H), 8.49 (s, 1H), 8.05 (d, J=2.6 Hz, 1H), 7.87 (d, J=5.0 Hz, 1H), 7.68 (s, 1H), 7.34 (d, J=6.6 Hz, 1H), 7.11 (t, J=9.3 Hz, 1H), 5.21 (t, J=13.8 Hz, 2H), 5.00 (d, J=16.0 Hz, 1H), 4.35 (d, J=11.5 Hz, 1H), 4.20 (t, J=11.9 Hz, 1H), 3.90 (s, 3H), 2.72 (d, J=4.8 Hz, 3H).

### Example 170: Synthesis of (6R)-6-[6-chloro-2-fluoro-3-(5-fluoro-2-methoxypyridine-3-sulfonamido)phenyl]-N-methyl-5H,6H,7H,8H-imidazo[1,5-a]pyridine-1-carboxamide and (6S)-6-[6-chloro-2-fluoro-3-(5-fluoro-2-methoxypyridine-3-sulfonamido)phenyl]-N-methyl-5H,6H,7H,8H-imidazo[1,5-a]pyridine-1-carboxamide (Compounds 155-1 & 155-2)

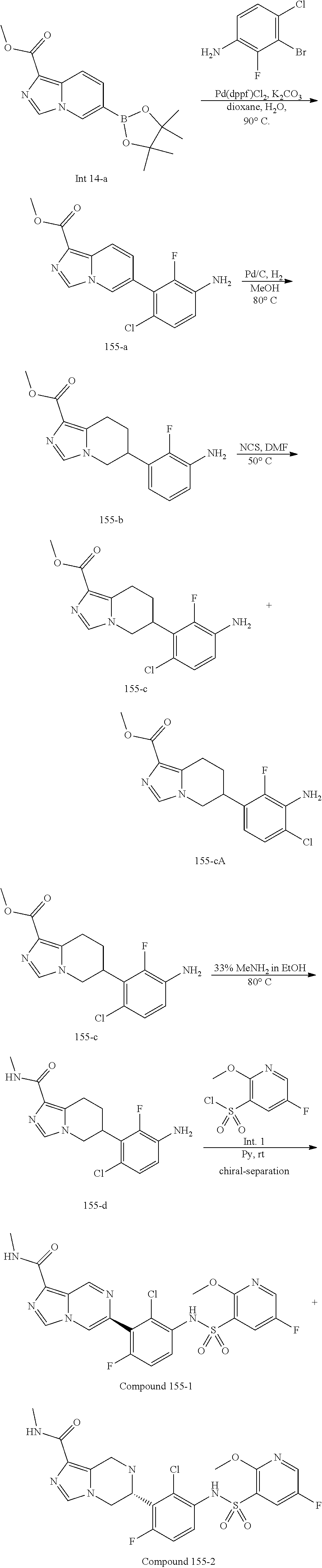

### Synthesis of 155-a: methyl 6-(3-amino-6-chloro-2-fluorophenyl)imidazo[1,5-a]pyridine-1-carboxylate

To a stirred mixture of methyl 6-(4,4,5,5-tetramethyl-1,3,2-dioxaborolan-2-yl)imidazo[1,5-a]pyridine-1-carboxylate (3.5 g, 12 mmol, 1 equiv) in dioxane (70 mL) and H₂O (10 mL) was added 3-bromo-4-chloro-2-fluoroaniline (2.8 g, 13 mmol, 1.1 equiv), Pd(dppf)Cl₂ (0.7 g, 0.9 mmol, 0.08 equiv) and K₂CO₃ (3.2 g, 23 mmol, 2 equiv). The reaction was stirred for 4 h at 90° C. under nitrogen atmosphere. The resulting solution was cooled and diluted with 50 mL of H₂O, then extracted with 3×50 mL of ethyl acetate. The combined organics were dried over anhydrous sodium sulfate and concentrated. The residue was purified by Flash-Prep-HPLC with the following conditions: Column, WelFlash™ C18-I, Spherical C18 20-40 m, 120 g; mobile phase: 30-80% MeCN/0.1% aqueous formic acid; to afford methyl 6-(3-amino-6-chloro-2-fluorophenyl)imidazo[1,5-a]pyridine-1-carboxylate (3 g, 81% yield) as a yellow solid.

LCMS (ES, m/z): [M+H]⁺: 320

### Synthesis of 155-b: methyl 6-(3-amino-2-fluorophenyl)-5H,6H,7H,8H-imidazo[1,5-a]pyridine-1-carboxylate

To a stirred mixture of methyl 6-(3-amino-6-chloro-2-fluorophenyl)imidazo[1,5-a]pyridine-1-carboxylate (2 g, 6.3 mmol, 1 equiv) in MeOH (120 mL) was added 10% Pd/C (2 g). The resulting suspension was stirred for 16 h at 80° C. under H₂ atmosphere (20 atm). The mixture was cooled and the solids removed by filtration. The filtrate was concentrated, and the residue purified by Flash-Prep-HPLC with the following conditions: Column, WelFlash™ C18-I, Spherical C18 20-40 m, 120 g; mobile phase: 5-55% MeCN/0.1% aqueous formic acid; to afford methyl 6-(3-amino-2-fluorophenyl)-5H,6H,7H,8H-imidazo[1,5-a]pyridine-1-carboxylate (807 mg, 45% yield) as a white solid.

LCMS (ES, m/z): [M+H]⁺: 290

### Synthesis of 155-c and 155-cA: methyl 6-(3-amino-6-chloro-2-fluorophenyl)-5H,6H,7H,8H-imidazo[1,5-a]pyridine-1-carboxylate and 6-(3-amino-4-chloro-2-fluorophenyl)-5H,6H,7H,8H-imidazo[1,5-a]pyridine-1-carboxylate

To a stirred mixture of methyl 6-(3-amino-2-fluorophenyl)-5H,6H,7H,8H-imidazo[1,5-a]pyridine-1-carboxylate (807 mg, 2.8 mmol, 1 equiv) in DMF (55 mL) was added NCS (410 mg, 3.1 mmol, 1.1 equiv). The reaction was stirred for 12 h at 50° C., then cooled and diluted with 20 mL of H₂O. The resulting mixture was extracted with 3×20 mL of ethyl acetate. The combined organics were washed with brine (2×100 mL), dried over anhydrous sodium sulfate and concentrated. The residue was purified by prep-HPLC with the following conditions: Column, welch Vltimate XB-C18, 50×250 mm, 10 μm; Mobile Phase: 16-33% MeCN/0.1% aqueous formic acid; to afford methyl 6-(3-amino-6-chloro-2-fluorophenyl)-5H,6H,7H,8H-imidazo[1,5-a]pyridine-1-carboxylate (530 mg, 59% yield) and 6-(3-amino-4-chloro-2-fluorophenyl)-5H,6H,7H,8H-imidazo[1,5-a]pyridine-1-carboxylate (237 mg, 26% yield) as white solids.

LCMS (ES, m/z): [M+H]⁺: 324

### Synthesis of 155-d: 6-(3-amino-6-chloro-2-fluorophenyl)-N-methyl-5H,6H,7H,8H-imidazo[1,5-a]pyridine-1-carboxamide

Into a 40 mL vial was placed methyl 6-(3-amino-6-chloro-2-fluorophenyl)-5H,6H,7H,8H-imidazo[1,5-a]pyridine-1-carboxylate (287 mg, 0.9 mmol, 1 equiv) and 33% methylamine solution in EtOH (20 mL). The solution was stirred for 3 days at 80° C. then concentrated to give crude 6-(3-amino-6-chloro-2-fluorophenyl)-N-methyl-5H,6H,7H,8H-imidazo[1,5-a]pyridine-1-carboxamide (287 mg) as brown oil which was used in next step directly without further purification.

LCMS (ES, m/z): [M+H]⁺: 323

### Synthesis of Compounds 155-1 & 155-2: (6R)-6-[6-chloro-2-fluoro-3-(5-fluoro-2-methoxypyridine-3-sulfonamido)phenyl]-N-methyl-5H,6H,7H,8H-imidazo[1,5-a]pyridine-1-carboxamide and (6S)-6-[6-chloro-2-fluoro-3-(5-fluoro-2-methoxypyridine-3-sulfonamido)phenyl]-N-methyl-5H,6H,7H,8H-imidazo[1,5-a]pyridine-1-carboxamide

Into a 40 mL vial was placed 6-(3-amino-6-chloro-2-fluorophenyl)-N-methyl-5H,6H,7H,8H-imidazo[1,5-a]pyridine-1-carboxamide (287 mg, 0.9 mmol, 1 equiv), pyridine (5 mL) and 5-fluoro-2-methoxypyridine-3-sulfonyl chloride (602 mg, 2.7 mmol, 3 equiv). The solution was stirred for 2 h then diluted with water (10 mL). The resulting mixture was extracted with EA (3×10 mL), and the combined organics washed with brine (2×5 mL), dried over anhydrous Na₂SO₄ then concentrated. The residue was purified by prep-HPLC with the following conditions: Column, welch Vltimate XB-C18, 50×250 mm, 10 μm: Mobile Phase: 20-50% MeCN/0.1% aqueous formic acid to give the racemic product. The enantiomers were separated by chiral-prep-HPLC with the following conditions: Column, CHIRALPAK IE, 3*25 cm, 5 μm; Mobile Phase: 5% MeOH/MTBE; Flow rate: 35 mL/min; to give (6R)-6-[6-chloro-2-fluoro-3-(5-fluoro-2-methoxypyridine-3-sulfonamido)phenyl]-N-methyl-5H,6H,7H,8H-imidazo[1,5-a]pyridine-1-carboxamide (retention time 22 mins, 50 mg, 11% yield, stereochemistry randomly assigned) and (6S)-6-[6-chloro-2-fluoro-3-(5-fluoro-2-methoxypyridine-3-sulfonamido) phenyl]-N-methyl-5H,6H,7H,8H-imidazo[1,5-a]pyridine-1-carboxamide (retention time 28 mins, 50 mg, 11% yield, stereochemistry assigned as opposite to other isomer) as white solids.

LCMS (ES, m/z): [M+H]⁺: 512 for both isomers

### (6R)-6-[6-chloro-2-fluoro-3-(5-fluoro-2-methoxypyridine-3-sulfonamido)phenyl]-N-methyl-5H,6H,7H,8H-imidazo[1,5-a]pyridine-1-carboxamide

¹H NMR (300 MHz, DMSO-d₆) δ 10.50 (s, 1H), 8.38 (d, J=3.0 Hz, 1H), 7.95 (dd, J=7.5, 3.0 Hz, 1H), 7.75 (q, J=4.7 Hz, 1H), 7.57 (s, 1H), 7.25-7.22 (m, 2H), 4.26 (dd, J=12.3, 5.2 Hz, 1H), 4.02 (t, J=11.9 Hz, 1H), 3.85 (s, 3H), 3.63 (m, 2H), 2.85 (ddd, J=17.7, 11.7, 6.0 Hz, 1H), 2.72 (d, J=4.7 Hz, 3H), 2.16 (d, J=13.0 Hz, 1H), 1.96 (s, 1H).

### (6S)-6-[6-chloro-2-fluoro-3-(5-fluoro-2-methoxypyridine-3-sulfonamido)phenyl]-N-methyl-5H,6H,7H,8H-imidazo[1,5-a]pyridine-1-carboxamide

¹H NMR (300 MHz, DMSO-d₆) δ 10.46 (s, 1H), 8.43 (s, 1H), 7.98 (d, J=7.4 Hz, 1H), 7.75 (d, J=4.9 Hz, 1H), 7.56 (s, 1H), 7.26 (d, J=6.5 Hz, 2H), 4.26 (dd, J=12.3, 5.2 Hz, 1H), 4.00 (t, J=11.9 Hz, 1H), 3.87 (s, 3H), 3.64 (s, 1H), 2.94-2.79 (m, 1H), 2.72 (d, J=4.7 Hz, 3H), 2.54 (s, 1H), 2.19-2.05 (m, 1H), 1.96 (s, 1H).

### Example 171: Synthesis of N-[2,4-difluoro-3-[1-(4-phenyl-1H-imidazol-2-yl)imidazo[1,5-a]pyrazin-6-yl]phenyl]-5-fluoro-2-methoxypyridine-3-sulfonamide (Compound 156)

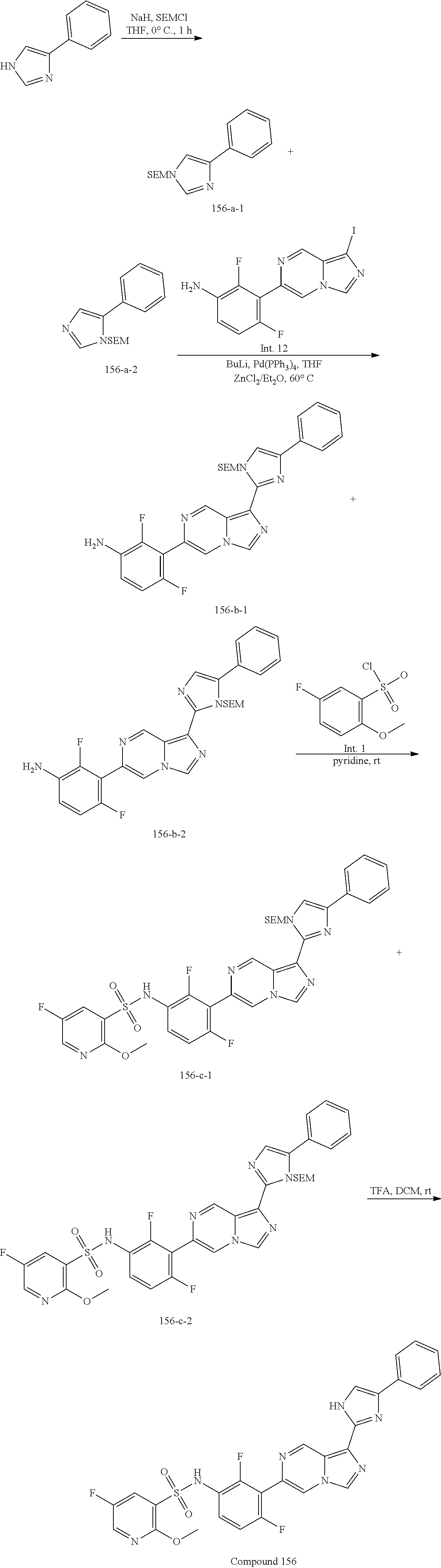

### Synthesis of 156-a: 5-phenyl-1-[[2-(trimethylsilyl)ethoxy]methyl]imidazole and 4-phenyl-1-((2-(trimethylsilyl) ethoxy)methyl)-1H-imidazole

To a stirred solution of 4-phenyl-1H-imidazole (4 g, 28 mmol, 1 equiv) in THF (140 mL) was added NaH (2 g, 55 mmol, 2 equiv, 60%) in portions at 0° C. To this was then added SEMCI (6.9 g, 42 mmol, 1.5 equiv) dropwise at 0° C. The reaction was stirred for additional 1 h in the ice bath, then quenched with water/ice (200 mL). The resulting mixture was extracted with EtOAc (3×200 mL), and the combined organics were washed with water (3×100 mL), dried over anhydrous Na₂SO₄, then concentrated. The residue was purified by silica gel column chromatography, eluting with PE/EtOAc (1:1) to afford 5-phenyl-1-[[2-(trimethylsilyl)ethoxy]methyl]imidazole and 4-phenyl-1-((2-(trimethylsilyl) ethoxy)methyl)-1H-imidazole mixture (6.9 g, 91% yield) as a yellow oil.

LCMS (ES, m/z): [M+H]⁺: 275

### Synthesis of 156-b: 2,4-difluoro-3-[1-(4-phenyl-1-[[2-(trimethylsilyl)ethoxy]methyl]imidazol-2-yl)imidazo[1,5-a]pyrazin-6-yl]aniline and 2,4-difluoro-3-[1-(5-phenyl-1-[[2-(trimethylsilylethoxy]methyl]imidazol-2-yl)imidazo[1,5-a]pyrazin-6-yl]aniline

To a stirred solution of 4-phenyl-1-((2-(trimethylsilyl)ethoxy)methyl)-1H-imidazole and 5-phenyl-1-((2-(trimethylsilyl)ethoxy)methyl)-1H-imidazole mixture (1.4 g, 5 mmol, 5 equiv) in THE (8 mL) was added n-BuLi in hexanes (32 mL, 81 mmol, 5 equiv, 2.5 M) dropwise at −78° C. and this was stirred at low temperature for 30 min. To the solution was added ZnCl₂ (5 mL, 5 mmol, 5 equiv, 1 M in Et₂O) dropwise at −78° C., and the resulting mixture was stirred to room temperature for 30 min. Pd(PPh₃)₄ (230 mg, 0.2 mmol, 0.2 equiv) and 2,4-difluoro-3-[1-iodoimidazo[1,5-a]pyrazin-6-yl]aniline (372 mg, 1 mmol, 1 equiv, in THF) were added, and this mixture heated to 60° C. for 1 hr. The reaction was cooled to 0° C. and quenched with aqueous NH₄Cl (saturated, 30 ml). The resulting mixture was extracted with EA (3×30 ml), and the combined organics washed with brine (30 mL), dried over anhydrous Na₂SO₄, then concentrated. The residue was purified by silica gel column chromatography, eluting with PE/EA (1/1) to afford 2,4-difluoro-3-[1-(4-phenyl-1-[[2-(trimethylsilyl)ethoxy]methyl]imidazol-2-yl)imidazo[1,5-a]pyrazin-6-yl]aniline and 2,4-difluoro-3-[1-(5-phenyl-1-[[2-(trimethylsilyl)ethoxy]methyl]imidazol-2-yl)imidazo[1,5-a]pyrazin-6-yl]aniline mixture (290 mg, 66% yield) as a yellow solid.

LCMS (ES, m/z): [M+H]⁺: 519

### Synthesis of 156-c: N-[2,4-difluoro-3-[1-(4-phenyl-1-[[2-(trimethylsilyl)ethoxy]methyl]imidazol-2-yl) imidazo[1,5-a]pyrazin-6-yl]phenyl]-5-fluoro-2-methoxypyridine-3-sulfonamide and N-[2,4-difluoro-3-[1-(5-phenyl-1-[[2-(trimethylsilyl)ethoxy]methyl]imidazol-2-yl) imidazo[1,5-a]pyrazin-6-yl]phenyl]-5-fluoro-2-methoxypyridine-3-sulfonamide

To a stirred solution of 2,4-difluoro-3-[1-(4-phenyl-1-[[2-(trimethylsilyl)ethoxy]methyl]imidazol-2-yl)imidazo[1,5-a]pyrazin-6-yl]aniline and 2,4-difluoro-3-[1-(5-phenyl-1-[[2-(trimethylsilyl)ethoxy]methyl]imidazol-2-yl)imidazo[1,5-a]pyrazin-6-yl]aniline mixture (100 mg, 0.2 mmol, 1 equiv) in pyridine (3 mL) was added 5-fluoro-2-methoxypyridine-3-sulfonyl chloride (65 mg, 0.3 mmol, 1.5 equiv). The resulting mixture was stirred for 2 h then concentrated. The residue was purified by Flash-Prep-HPLC with the following conditions: Column, WelFlash™ C18-I, Spherical C18 20-40 m, 120 g; mobile phase: 20-70% MeCN/0.1% aqueous formic acid; to give N-[2,4-difluoro-3-[1-(4-phenyl-1-[[2-(trimethylsilyl)ethoxy]methyl]imidazol-2-yl)imidazo[1,5-a]pyrazin-6-yl]phenyl]-5-fluoro-2-methoxypyridine-3-sulfonamide and N-[2,4-difluoro-3-[1-(5-phenyl-1-[[2-(trimethylsilyl)ethoxy]methyl]imidazol-2-yl) imidazo[1,5-a]pyrazin-6-yl]phenyl]-5-fluoro-2-methoxypyridine-3-sulfonamide mixture (95 mg, 70% yield) as a yellow solid.

LCMS (ES, m/z): [M+H]⁺: 708

### Synthesis of Compound 156: N-[2,4-difluoro-3-[1-(4-phenyl-1H-imidazol-2-yl)imidazo[1,5-a]pyrazin-6-yl]phenyl]-5-fluoro-2-methoxypyridine-3-sulfonamide

To a stirred solution of N-[2,4-difluoro-3-[1-(5-phenyl-1-[[2-(trimethylsilyl)ethoxy]methyl]imidazol-2-yl)imidazo[1,5-a]pyrazin-6-yl]phenyl]-5-fluoro-2-methoxypyridine-3-sulfonamide and N-[2,4-difluoro-3-[1-(4-phenyl-1-[[2-(trimethylsilyl)ethoxy]methyl]imidazol-2-yl)imidazo[1,5-a]pyrazin-6-yl]phenyl]-5-fluoro-2-methoxypyridine-3-sulfonamide (95 mg, 0.1 mmol, 1 equiv, mixture) in DCM (6 mL) was added TFA (2 mL). The resulting mixture was stirred for 5 h then concentrated. The residue was purified by prep-HPLC with the following conditions: Column, welch Vltimate XB-C18, 50×250 mm, 10 μm; Mobile Phase: 5-60% MeCN/0.1% aqueous formic acid; to give N-[2,4-difluoro-3-[1-(4-phenyl-1H-imidazol-2-yl)imidazo[1,5-a]pyrazin-6-yl]phenyl]-5-fluoro-2-methoxypyridine-3-sulfonamide (60 mg, 77% yield) as a yellow solid.

LCMS (ES, m/z): [M+H]⁺: 578

¹H NMR (300 MHz, DMSO-d₆) δ 13.01 (s, 1H), 10.45 (s, 1H), 9.80-9.49 (s, 1H), 8.70 (s, 1H), 8.58 (d, 1H), 8.44 (d, J=3.0 Hz, 1H), 8.10-7.83 (m, 3H), 7.80-7.52 (d, J=7.7 Hz, 1H), 7.40 (m, 3H), 7.22 (m, 2H), 3.91 (s, 3H).

### Example 172: Synthesis of 6-[3-(5-chloro-2-methoxypyridine-3-sulfonamido)-2,6-difluorophenyl]-N-isopropylimidazo[1,5-a]pyrazine-1-carboxamide (Compound 157)

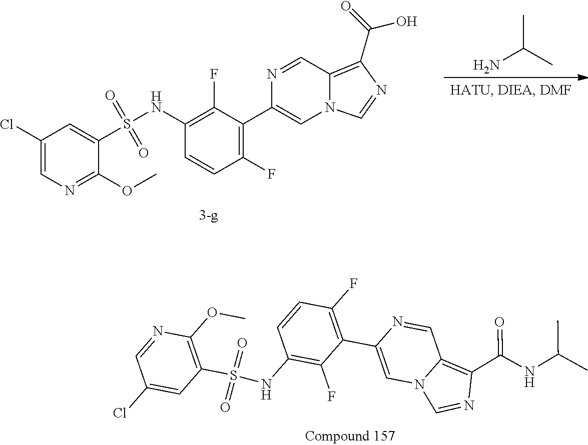

### Synthesis of Compound 157: 6-[3-(5-chloro-2-methoxypyridine-3-sulfonamido)-2,6-difluorophenyl]-N-isopropylimidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added isopropylamine (35 mg, 0.6 mmol, 1.5 equiv) and HATU (230 mg, 0.6 mmol, 1.5 equiv) at room temperature. The resulting mixture was stirred for 1 h then diluted with H₂O (50 mL). This was extracted with EA (3×50 mL), and the combined organics were washed with brine (3×30 mL), dried over anhydrous Na₂SO₄ and concentrated. The crude product was purified by Prep-HPLC with following conditions: Column: Sunfire Prep C18 OBD, 50*250 mm 5 μm 10 nm; Mobile Phase: 15-50% MeCN/0.1% aqueous formic acid; to afford 6-[3-(5-chloro-2-methoxypyridine-3-sulfonamido)-2,6-difluorophenyl]-N-isopropylimidazo[1,5-a]pyrazine-1-carboxamide (90 mg, 42% yield) as a white solid.

LCMS (ES, m/z): [M+H]⁺: 537

¹H NMR (300 MHz, DMSO-d₆) δ 10.45 (s, 1H), 9.51 (d, J=1.6 Hz, 1H), 8.66 (d, J=4.0 Hz, 2H), 8.50 (d, J=2.6 Hz, 1H), 8.23-8.03 (m, 2H), 7.42 (td, J=8.8, 5.8 Hz, 1H), 7.25 (td, J=9.1, 1.6 Hz, 1H), 4.25-4.08 (m, 1H), 3.92 (s, 3H), 1.22 (d, J=6.6 Hz, 6H).

### Example 173: Synthesis of 6-[3-(5-chloro-2-methoxypyridine-3-sulfonamido)-2,6-difluorophenyl]-N-(2-methylpropyl)imidazo[1,5-a]pyrazine-1-carboxamide (Compound 158)

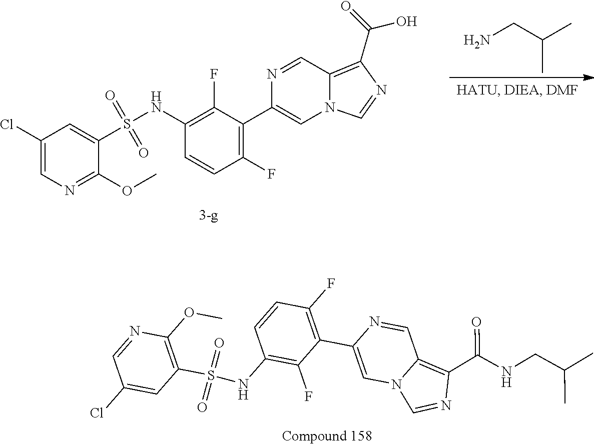

### Synthesis of Compound 158: 6-[3-(5-chloro-2-methoxypyridine-3-sulfonamido)-2,6-difluorophenyl]-N-(2-methylpropyl)imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added isobutylamine (44 mg, 0.6 mmol, 1.5 equiv) and HATU (230 mg, 0.6 mmol, 1.5 equiv). The reaction was stirred for 1 h at room temperature. The resulting mixture was diluted with H₂O (50 mL) and extracted with EA (3×50 mL). The combined organics were washed with brine (3×30 mL), dried over anhydrous Na₂SO₄ and concentrated. The crude product was purified by Prep-HPLC with following conditions: Column: Sunfire Prep C18 OBD, 50*250 mm 5 μm 10 nm; Mobile Phase: 15-50% MeCN/0.1% aqueous formic acid; to afford 6-[3-(5-chloro-2-methoxypyridine-3-sulfonamido)-2,6-difluorophenyl]-N-(2-methylpropyl)imidazo[1,5-a]pyrazine-1-carboxamide (95 mg, 42% yield) as a white solid.

LCMS (ES, m/z): [M+H]⁺: 551

¹H NMR (300 MHz, DMSO-d₆) δ 10.45 (s, 1H), 9.51 (d, J=1.6 Hz, 1H), 8.67 (d, J=3.3 Hz, 2H), 8.50 (d, J=2.6 Hz, 1H), 8.41 (t, J=6.2 Hz, 1H), 8.09 (d, J=2.6 Hz, 1H), 7.42 (td, J=8.8, 5.8 Hz, 1H), 7.25 (td, J=9.1, 1.6 Hz, 1H), 3.92 (s, 3H), 3.23-3.09 (m, 2H), 1.89 (dq, J=13.6, 6.9 Hz, 1H), 0.90 (d, J=6.7 Hz, 6H).

### Example 174: Synthesis of 6-[3-(5-chloro-2-methoxypyridine-3-sulfonamido)-2,6-difluorophenyl]-N-(2,2,2-trifluoroethyl)imidazo[1,5-a]pyrazine-1-carboxamide (Compound 159)

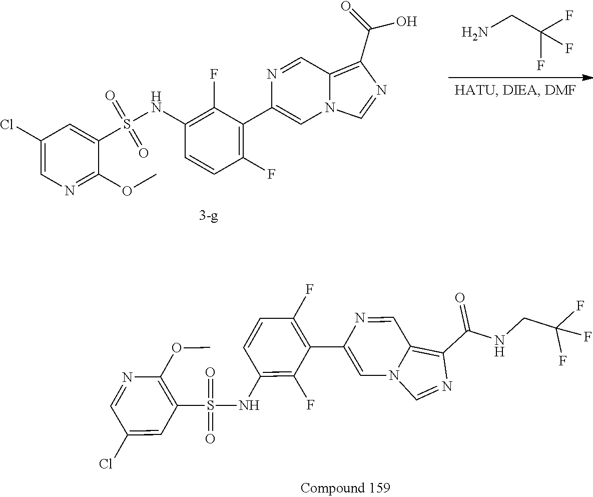 [SMILES: COc1ncc(Cl)cc1S(=O)(=O)Nc1ccc(F)c(-c2cn3cnc(C(=O)O)c3cn2)c1F ; NCC(F)(F)F ; COc1ncc(Cl)cc1S(=O)(=O)Nc1ccc(F)c(-c2cn3cnc(C(=O)NCC(F)(F)F)c3cn2)c1F]

### Synthesis of Compound 160: 6-[3-(5-chloro-2-methoxypyridine-3-sulfonamido)-2,6-difluorophenyl]-N-(2,2,2-trifluoroethyl)imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added 2,2,2-trifluoroethylamine (59 mg, 0.6 mmol, 1.5 equiv) and HATU (230 mg, 0.6 mmol, 1.5 equiv). The reaction was stirred for 1 h at room temperature, then diluted with H₂O (50 mL). The resulting mixture was extracted with EA (3×50 mL) and the combined organics were washed with brine (3×30 mL), dried over anhydrous Na₂SO₄ and concentrated. The crude product was purified by Prep-HPLC with following conditions: Column: Sunfire Prep C18 OBD, 50*250 mm 5 μm 10 nm; Mobile Phase: 10-30% MeCN/0.1% aqueous formic acid; to afford 6-[3-(5-chloro-2-methoxypyridine-3-sulfonamido)-2,6-difluorophenyl]-N-(2,2,2-trifluoroethyl)imidazo[1,5-a]pyrazine-1-carboxamide (105 mg, 45% yield) as a white solid.

LCMS (ES, m/z): [M+H]⁺: 577

¹H NMR (300 MHz, DMSO-d₆) δ 10.45 (s, 1H), 9.52 (d, J=1.6 Hz, 1H), 9.03 (t, J=6.6 Hz, 1H), 8.72 (d, J=2.6 Hz, 2H), 8.51 (d, J=2.6 Hz, 1H), 8.09 (d, J=2.6 Hz, 1H), 7.43 (td, J=8.8, 5.8 Hz, 1H), 7.26 (td, J=9.1, 1.6 Hz, 1H), 4.10 (dq, 2H), 3.92 (s, 3H).

### Example 175: Synthesis of 6-[3-(5-chloro-2-methoxypyridine-3-sulfonamido)-2,6-difluorophenyl]-5-fluoro-N-methylimidazo[1,5-a]pyrazine-1-carboxamide (Compound 160)

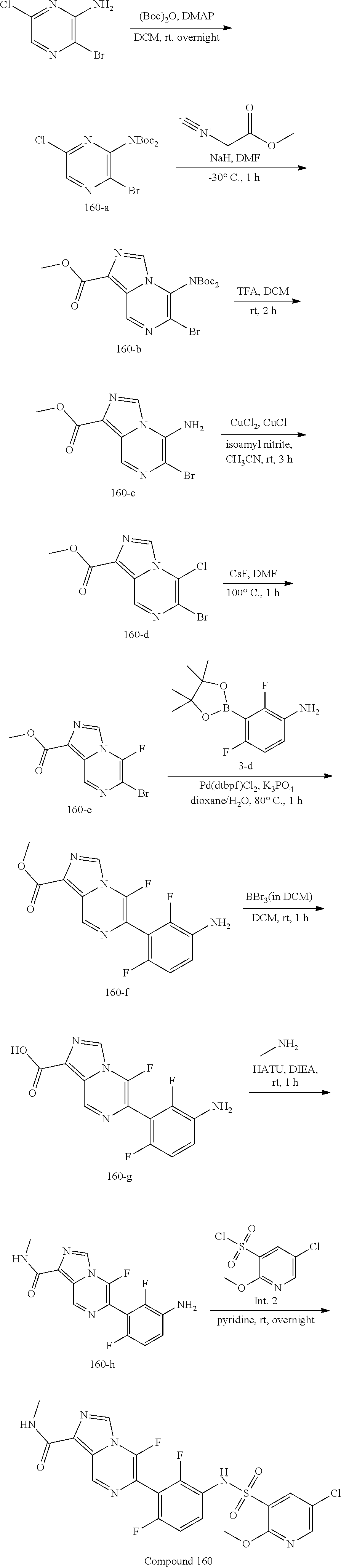

### Synthesis of 160-a: tert-butyl N-(3-bromo-6-chloropyrazin-2-yl)-N-(tert-butoxycarbonyl)carbamate

To a stirred solution of 3-bromo-6-chloropyrazin-2-amine (20 g, 96 mmol, 1 equiv) in DCM (400 mL) was added DMAP (5.8 g, 48 mmol, 0.5 equiv) and (Boc)₂O (52 g, 240 mmol, 2.5 equiv). The resulting mixture was stirred overnight then washed with brine (2×600 mL), dried over anhydrous Na₂SO₄ and concentrated. The residue was purified by silica gel column chromatography, eluting with PE/EtOAc (10:1) to afford tert-butyl N-(3-bromo-6-chloropyrazin-2-yl)-N-(tert-butoxycarbonyl)carbamate (34 g, 87% yield) as an off-white solid.

LCMS (ES, m/z): [M+H]⁺: 408

### Synthesis of 160-b: methyl 5-[bis(tert-butoxycarbonyl)amino]-6-bromoimidazo[1,5-a]pyrazine-1-carboxylate

To NaH (4 g, 100 mmol, 1.2 equiv, 60% in oil) in DMF (400 mL) was added methyl 2-isocyanoacetate (9.9 g, 100 mmol, 1.2 equiv) dropwise at −30° C. and the reaction was stirred for 20 mins. A solution of tert-butyl N-(3-bromo-6-chloropyrazin-2-yl)-N-(tert-butoxycarbonyl)carbamate (34 g, 83.2 mmol, 1 equiv) in DMF (20 mL) was then added dropwise at −30° C. and the mixture stirred for 1 h at this temperature. The reaction was quenched with NH₄Cl (aq.) at 0° C. and extracted with EtOAc (3×500 mL). The combined organics were washed with water (3×600 mL), dried over anhydrous Na₂SO₄ and concentrated. The residue was purified by silica gel column chromatography, eluting with PE/EtOAc (1:1) to afford methyl 5-[bis(tert-butoxycarbonyl)amino]-6-bromoimidazo[1,5-a]pyrazine-1-carboxylate (21 g, 54% yield) as a yellow solid.

LCMS (ES, m/z): [M+H]⁺: 471, 473

### Synthesis of 160-c: methyl 5-amino-6-bromoimidazo[1,5-a]pyrazine-1-carboxylate

To a stirred solution of methyl 5-[bis(tert-butoxycarbonyl)amino]-6-bromoimidazo[1,5-a]pyrazine-1-carboxylate (21 g, 44 mmol, 1 equiv) in DCM (100 mL) was added TFA (50 mL). The resulting mixture was stirred for 2 h at room temperature then concentrated. Diethyl ether was added and the precipitated solids were collected by filtration and washed with ethyl ether (2×100 mL). Drying gave methyl 5-amino-6-bromoimidazo[1,5-a]pyrazine-1-carboxylate (11 g, 91% yield) as a yellow solid.

LCMS (ES, m/z): [M+H]⁺: 271, 273

### Synthesis of 160-d: methyl 6-bromo-5-chloroimidazo[1,5-a]pyrazine-1-carboxylate

To a stirred solution of methyl 5-amino-6-bromoimidazo[1,5-a]pyrazine-1-carboxylate (3.3 g, 12 mmol, 1 equiv) in MeCN (40 mL) was added CuCl₂ (4.9 g, 36 mmol, 3 equiv) and CuCl (2.4 g, 24 mmol, 2 equiv). To this mixture was added isopentyl nitrite (2.8 g, 24 mmol, 2 equiv) dropwise at room temperature. The resulting mixture was stirred for 3 h then quenched with water/ice (100 mL). This was extracted with EtOAc (3×100 mL), and the combined organics were washed with brine (100 mL), dried over anhydrous Na₂SO₄ and concentrated. The residue was purified by silica gel column chromatography, eluting with PE/EtOAc (2:1) to afford methyl 6-bromo-5-chloroimidazo[1,5-a]pyrazine-1-carboxylate (1.2 g, 34% yield) as a light yellow solid.

LCMS (ES, m/z): [M+H]⁺: 290, 292

### Synthesis of 160-e: methyl 6-bromo-5-fluoroimidazo[1,5-a]pyrazine-1-carboxylate

To a stirred solution of methyl 6-bromo-5-chloroimidazo[1,5-a]pyrazine-1-carboxylate (1.2 g, 4 mmol, 1 equiv) in DMF (40 mL) was added CsF (3.1 g, 20 mmol, 5 equiv). The reaction was stirred for 1 h at 100° C., then cooled to 0° C. and quenched with 1 M HCl. The resulting mixture was diluted with water (200 mL) and extracted with EtOAc (3×200 mL). The combined organics were washed with water (3×200 mL), dried over anhydrous Na₂SO₄ and concentrated. The residue was purified by silica gel column chromatography, eluting with PE/EtOAc (1:1) to afford methyl 6-bromo-5-fluoroimidazo[1,5-a]pyrazine-1-carboxylate (500 mg, 31% yield) as a yellow solid.

LCMS (ES, m/z): [M+H]⁺: 274, 276

### Synthesis of 160-f: methyl 6-(3-amino-2,6-difluorophenyl)-5-fluoroimidazo[1,5-a]pyrazine-1-carboxylate

To a stirred mixture of methyl 6-bromo-5-fluoroimidazo[1,5-a]pyrazine-1-carboxylate (500 mg, 1.8 mmol, 1 equiv) and 2,4-difluoro-3-(4,4,5,5-tetramethyl-1,3,2-dioxaborolan-2-yl)aniline (698 mg, 2.7 mmol, 1.5 equiv) in dioxane (15 mL) and H₂O (1.5 mL) was added Pd(dtbpf)Cl₂ (119 mg, 0.18 mmol, 0.1 equiv) and K₃PO₄ (774 mg, 3.6 mmol, 2 equiv) at room temperature under nitrogen atmosphere. The reaction was stirred for 1 h at 80° C. then cooled to 0° C. and quenched with water/ice (100 mL). The resulting mixture was extracted with EtOAc (3×100 mL) and the combined organics were washed with brine (100 mL), dried over anhydrous Na₂SO₄ and concentrated. The residue was purified by silica gel column chromatography, eluting with PE/EtOAc (1:1) to afford methyl 6-(3-amino-2,6-difluorophenyl)-5-fluoroimidazo[1,5-a]pyrazine-1-carboxylate (410 mg, 69% yield) as a yellow solid.

LCMS (ES, m/z): [M+H]⁺: 323

### Synthesis of 160-g: 6-(3-amino-2,6-difluorophenyl)-5-fluoroimidazo[1,5-a]pyrazine-1-carboxylic acid

To a stirred mixture of methyl 6-(3-amino-2,6-difluorophenyl)-5-fluoroimidazo[1,5-a]pyrazine-1-carboxylate (420 mg, 1.3 mmol, 1 equiv) in DCM (10 mL) was added BBr₃ (2 mL) at room temperature. The resulting mixture was stirred for 3 h then concentrated. This resulted in 6-(3-amino-2,6-difluorophenyl)-5-fluoroimidazo[1,5-a]pyrazine-1-carboxylic acid (500 mg, 87% yield) as a brown solid. The crude product was used in the next step directly without further purification.

LCMS (ES, m/z): [M+H]⁺: 309

### Synthesis of 160-h: 6-(3-amino-2,6-difluorophenyl)-5-fluoro-N-methylimidazo[1,5-a]pyrazine-1-carboxamide

To a stirred mixture of 6-(3-amino-2,6-difluorophenyl)-5-fluoroimidazo[1,5-a]pyrazine-1-carboxylic acid (500 mg, 1.6 mmol, 1 equiv) in DMF (8 mL) was added HATU (925 mg, 2.4 mmol, 1.5 equiv), DIEA (629 mg, 4.9 mmol, 3 equiv) and methylamine hydrochloride (219 mg, 3.2 mmol, 2 equiv). The reaction was stirred for 1 h, then quenched by the addition of MeOH (3 mL). Concentration gave a residue which was purified by Flash-Prep-HPLC with the following conditions: Column, WelFlash™ C18-I, Spherical C18 20-m, 120 g; mobile phase: 5-50% MeCN/0.1% aqueous TFA; to give 6-(3-amino-2,6-difluorophenyl)-5-fluoro-N-methylimidazo[1,5-a]pyrazine-1-carboxamide (250 mg, 48% yield) as a yellow solid.

LCMS (ES, m/z): [M+H]⁺: 322

### Synthesis of Compound 160: 6-[3-(5-chloro-2-methoxypyridine-3-sulfonamido)-2,6-difluorophenyl]-5-fluoro-N-methylimidazo[1,5-a]pyrazine-1-carboxamide

To a stirred solution of 6-(3-amino-2,6-difluorophenyl)-5-fluoro-N-methylimidazo[1,5-a]pyrazine-1-carboxamide (80 mg, 0.25 mmol, 1 equiv) in pyridine (3 mL) was added 5-chloro-2-methoxypyridine-3-sulfonyl chloride (120 mg, 0.5 mmol, 2 equiv) in portions at room temperature. The resulting mixture was stirred overnight then concentrated. The residue was purified by prep-HPLC with the following conditions: Column, welch Vltimate XB-C18, 50×250 mm, 10 m; Mobile Phase: 20-55% MeCN/0.1% aqueous formic acid; to give 6-[3-(5-chloro-2-methoxypyridine-3-sulfonamido)-2,6-difluorophenyl]-5-fluoro-N-methylimidazo[1,5-a]pyrazine-1-carboxamide (50 mg, 38% yield) as an off-white solid.

LCMS (ES, m/z): [M+H]⁺: 527

¹H NMR (300 MHz, DMSO-d₆) δ 10.50 (s, 1H), 9.36 (d, J=2.9 Hz, 1H), 8.92 (s, 1H), 8.53 (dd, J=11.6, 3.7 Hz, 2H), 8.09 (d, J=2.6 Hz, 1H), 7.52 (td, J=8.9, 5.8 Hz, 1H), 7.31 (td, J=9.0, 1.6 Hz, 1H), 3.91 (s, 3H), 2.85 (d, J=4.7 Hz, 3H).

### Example 176: Synthesis of 6-[3-(5-chloro-2-methoxypyridine-3-sulfonamido)-2,6-difluorophenyl]-N-(2-hydroxyethyl)imidazo[1,5-a]pyrazine-1-carboxamide (Compound 161)

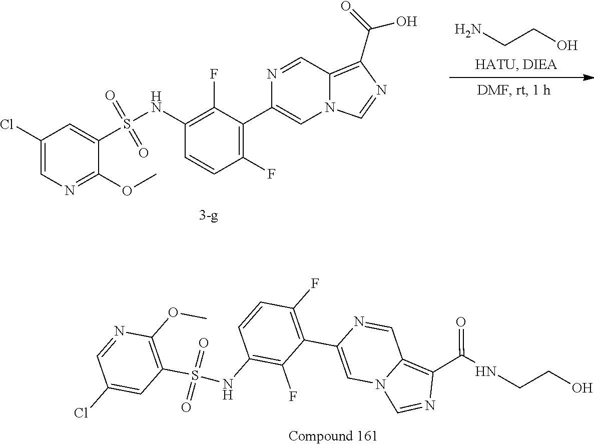

### Synthesis of Compound 161: 6-[3-(5-chloro-2-methoxypyridine-3-sulfonamido)-2,6-difluorophenyl]-N-(2-hydroxyethyl)imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added ethanolamine (36 mg, 0.6 mmol, 1.5 equiv) and HATU (230 mg, 0.6 mmol, 1.5 equiv) at room temperature. The reaction was stirred for 1 h, then diluted with H₂O (50 mL). The resulting mixture was extracted with EA (3×50 mL), and the combined organics were washed with brine (3×30 mL), dried over anhydrous Na₂SO₄ and concentrated. The crude product was purified by Prep-HPLC with following conditions: Column: welch Vltimate XB-C18, 50×250 mm, 10 m; Mobile Phase: 10-30% MeCN/0.1% aqueous formic acid; to afford 6-[3-(5-chloro-2-methoxypyridine-3-sulfonamido)-2,6-difluorophenyl]-N-(2-hydroxyethyl)imidazo[1,5-a]pyrazine-1-carboxamide (103 mg, 47% yield) as a white solid.

LCMS (ES, m/z): [M+H]⁺: 539

¹H NMR (300 MHz, DMSO-d₆) δ 10.45 (s, 1H), 9.51 (d, J=1.6 Hz, 1H), 8.72-8.60 (m, 2H), 8.51 (d, J=2.6 Hz, 1H), 8.29 (t, J=5.8 Hz, 1H), 8.09 (d, J=2.6 Hz, 1H), 7.42 (td, J=8.8, 5.8 Hz, 1H), 7.25 (td, J=9.0, 1.6 Hz, 1H), 4.80 (s, 1H), 3.92 (s, 3H), 3.56 (t, J=6.2 Hz, 2H), 3.41 (q, J=6.1 Hz, 2H).

### Example 177: Synthesis of 6-[3-(5-chloro-2-methoxypyridine-3-sulfonamido)-2,6-difluorophenyl]-N-(1-methylpyrazol-4-yl)imidazo[1,5-a]pyrazine-1-carboxamide (Compound 162)

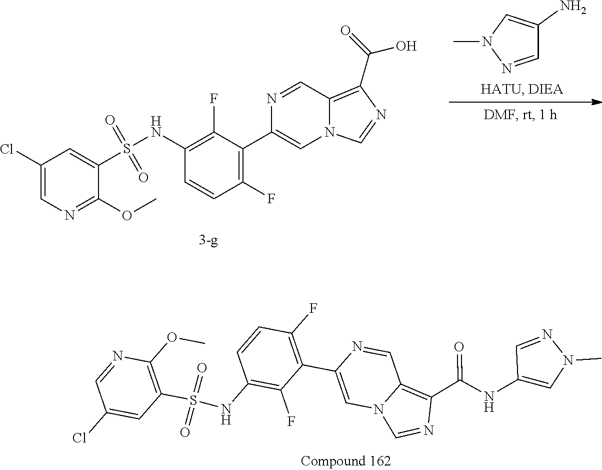

### Synthesis of Compound 162: 6-[3-(5-chloro-2-methoxypyridine-3-sulfonamido)-2,6-difluorophenyl]-N-(1-methylpyrazol-4-yl)imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added 1-methylpyrazol-4-amine (58 mg, 0.6 mmol, 1.5 equiv) and HATU (230 mg, 0.6 mmol, 1.5 equiv). The reaction was stirred for 1 h then diluted with H₂O (50 mL). The resulting mixture was extracted with EA (3×50 mL) and the combined organics were washed with brine (3×30 mL), dried over anhydrous Na₂SO₄ and concentrated. The crude product was purified by Prep-HPLC with following conditions: Column: welch Vltimate XB-C18, 50×250 mm, 10 m; Mobile Phase: 5-55% MeCN/0.1% aqueous formic acid; to afford 6-[3-(5-chloro-2-methoxypyridine-3-sulfonamido)-2,6-difluorophenyl]-N-(1-methylpyrazol-4-yl)imidazo[1,5-a]pyrazine-1-carboxamide (92 mg, 40% yield) as a white solid.

LCMS (ES, m/z): [M+H]⁺: 575

¹H NMR (300 MHz, DMSO-d₆) δ 10.65 (s, 1H), 10.46 (s, 1H), 9.58 (d, J=1.6 Hz, 1H), 8.73 (d, J=6.6 Hz, 2H), 8.51 (d, J=2.6 Hz, 1H), 8.17-7.97 (m, 2H), 7.72 (s, 1H), 7.43 (td, J=8.8, 5.8 Hz, 1H), 7.26 (td, J=9.1, 1.5 Hz, 1H), 3.93 (s, 3H), 3.83 (s, 3H).

### Example 178: Synthesis of 6-[3-(5-chloro-2-methoxypyridine-3-sulfonamido)-2,6-difluorophenyl]-N-(oxan-4-ylmethyl)imidazo[1,5-a]pyrazine-1-carboxamide (Compound 163)

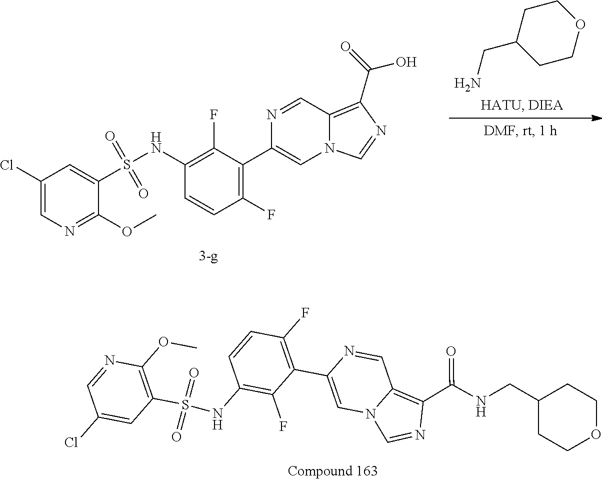 [SMILES: COc1ncc(Cl)cc1S(=O)(=O)Nc1ccc(F)c(-c2cn3cnc(C(=O)O)c3cn2)c1F ; NCC1CCOCC1 ; COc1ncc(Cl)cc1S(=O)(=O)Nc1ccc(F)c(-c2cn3cnc(C(=O)NCC4CCOCC4)c3cn2)c1F]

### Synthesis of Compound 163: 6-[3-(5-chloro-2-methoxypyridine-3-sulfonamido)-2,6-difluorophenyl]-N-(oxan-4-ylmethyl)imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added 1-(oxan-4-yl)methanamine (69 mg, 0.6 mmol, 1.5 equiv) and HATU (230 mg, 0.6 mmol, 1.5 equiv). The reaction was stirred for 1 h, then diluted with H₂O (50 mL). The resulting mixture was extracted with EA (3×50 mL), and the combined organics were washed with brine (3×30 mL), dried over anhydrous Na₂SO₄ and concentrated. The crude product was purified by Prep-HPLC with following conditions: Column, welch Vltimate XB-C18, 50×250 mm, 10 m; Mobile Phase: 25-60% MeCN/0.1% aqueous formic acid; to afford 6-[3-(5-chloro-2-methoxypyridine-3-sulfonamido)-2,6-difluorophenyl]-N-(oxan-4-ylmethyl)imidazo[1,5-a]pyrazine-1-carboxamide (88 mg, 37% yield) as a white solid.

LCMS (ES, m/z): [M+H]⁺: 593

¹H NMR (300 MHz, DMSO-d₆) δ 10.44 (s, 1H), 9.51 (d, J=1.6 Hz, 1H), 8.76-8.61 (m, 2H), 8.55-8.31 (m, 2H), 8.09 (d, J=2.6 Hz, 1H), 7.42 (td, J=8.8, 5.8 Hz, 1H), 7.25 (td, J=9.0, 1.6 Hz, 1H), 3.92 (s, 3H), 3.89-3.80 (m, 1H), 3.30-3.18 (m, 4H), 1.86 (ddd, J=11.3, 7.5, 4.1 Hz, 1H), 1.59 (dd, J=12.9, 3.7 Hz, 2H), 1.32-1.09 (m, 2H).

### Example 179: Synthesis of 6-[3-(5-chloro-2-methoxypyridine-3-sulfonamido)-2,6-difluorophenyl]-N-(2-methanesulfonylethyl)imidazo[1,5-a]pyrazine-1-carboxamide (Compound 164)

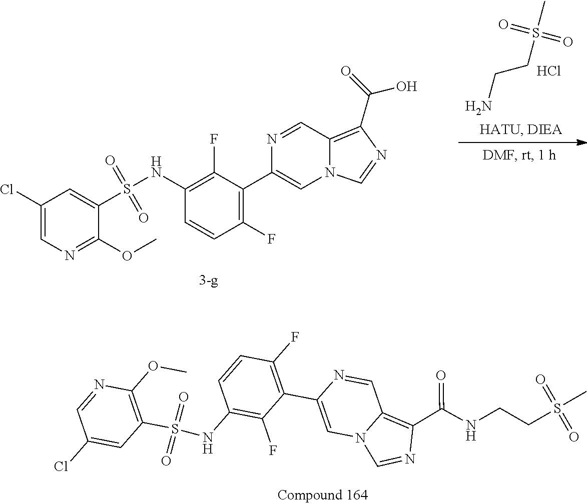

### Synthesis of Compound 164: 6-[3-(5-chloro-2-methoxypyridine-3-sulfonamido)-2,6-difluorophenyl]-N-(2-methanesulfonylethyl)imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added 2-methanesulfonylethanamine (74 mg, 0.6 mmol, 1.5 equiv) and HATU (230 mg, 0.6 mmol, 1.5 equiv). The resulting mixture was stirred for 1 h, then diluted with H₂O (50 mL). The resulting mixture was extracted with EA (3×50 mL) and the combined organics were washed with brine (3×30 mL), dried over anhydrous Na₂SO₄ and concentrated. The crude product was purified by Prep-HPLC with following conditions: Column, welch Vltimate XB-C18, 50×250 mm, 10 μm; Mobile Phase: 15-55% MeCN/0.1% aqueous formic acid; to afford 6-[3-(5-chloro-2-methoxypyridine-3-sulfonamido)-2,6-difluorophenyl]-N-(2-methanesulfonylethyl)imidazo[1,5-a]pyrazine-1-carboxamide (85 mg, 35% yield) as a white solid.

LCMS (ES, m/z): [M+H]⁺: 601

¹H NMR (300 MHz, DMSO-d₆) δ 10.47 (s, 1H), 9.52 (d, J=1.6 Hz, 1H), 8.81-8.56 (m, 3H), 8.44 (d, J=2.6 Hz, 1H), 8.07 (d, J=2.6 Hz, 1H), 7.38 (td, J=9.0, 5.9 Hz, 1H), 7.17 (t, J=9.1 Hz, 1H), 3.90 (s, 3H), 3.77 (q, J=6.6 Hz, 2H), 3.43 (t, J=6.9 Hz, 2H), 3.06 (s, 3H).

### Example 180: Synthesis of 6-[3-(5-chloro-2-methoxypyridine-3-sulfonamido)-2,6-difluorophenyl]-N-ethylimidazo[1,5-a]pyrazine-1-carboxamide (Compound 165)

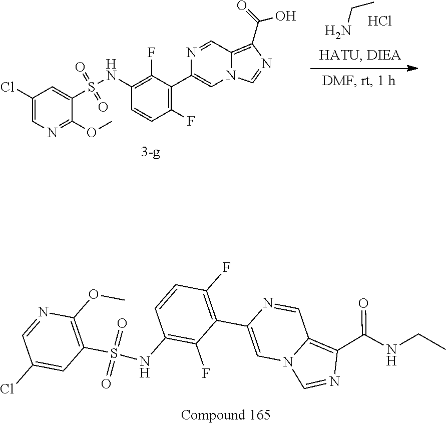

### Synthesis of Compound 165: 6-[3-(5-chloro-2-methoxypyridine-3-sulfonamido)-2,6-difluorophenyl]-N-ethylimidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added ethylamine hydrochloride (49 mg, 0.6 mmol, 1.5 equiv) and HATU (230 mg, 0.6 mmol, 1.5 equiv). The reaction was stirred for 1 h, then diluted with H₂O (50 mL). The resulting mixture was extracted with EA (3×50 mL) and the combined organics were washed with brine (3×30 mL), dried over anhydrous Na₂SO₄ and concentrated. The crude product was purified by Prep-HPLC with following conditions: Column: welch Vltimate XB-C18, 50×250 mm, 10 μm; Mobile Phase: 30-60% MeCN/0.1% aqueous formic acid; to afford 6-[3-(5-chloro-2-methoxypyridine-3-sulfonamido)-2,6-difluorophenyl]-N-ethylimidazo[1,5-a]pyrazine-1-carboxamide (87 mg, 41% yield) as a white solid.

LCMS (ES, m/z): [M+H]⁺: 523

¹H NMR (300 MHz, DMSO-d₆) δ 10.45 (s, 1H), 9.51 (d, J=1.6 Hz, 1H), 8.67 (d, J=5.5 Hz, 2H), 8.55-8.39 (m, 2H), 8.09 (d, J=2.6 Hz, 1H), 7.42 (td, J=8.8, 5.8 Hz, 1H), 7.25 (td, J=9.0, 1.6 Hz, 1H), 3.92 (s, 3H), 3.37-3.31 (m, 2H), 1.15 (t, J=7.1 Hz, 3H).

### Example 181: Synthesis of 6-[3-(5-chloro-2-methoxypyridine-3-sulfonamido)-2,6-difluorophenyl]-N-(cyclopropylmethyl)imidazo[1,5-a]pyrazine-1-carboxamide (Compound 166)

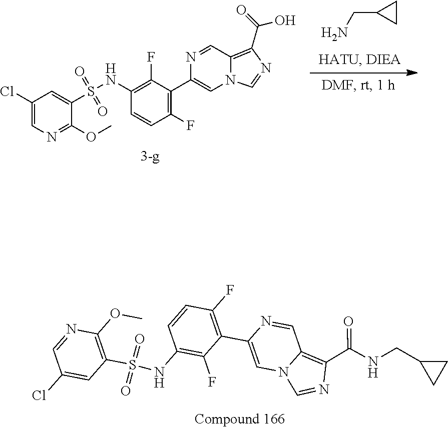 [SMILES: COc1ncc(Cl)cc1S(=O)(=O)Nc1ccc(F)c(-c2cn3cnc(C(=O)O)c3cn2)c1F ; NCC1CC1 ; COc1ncc(Cl)cc1S(=O)(=O)Nc1ccc(F)c(-c2cn3cnc(C(=O)NCC4CC4)c3cn2)c1F]

### Synthesis of Compound 166: 6-[3-(5-chloro-2-methoxypyridine-3-sulfonamido)-2,6-difluorophenyl]-N-(cyclopropylmethyl)imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMIF (5 mL) was added 1-cyclopropylmethylamine (43 mg, 0.6 mmol, 1.5 equiv) and HATU (230 mg, 0.6 mmol, 1.5 equiv). The reaction was stirred for 1 h, then diluted with H₂O (50 mL). The resulting mixture was extracted with EA (3×50 mL), and the combined organics were washed with brine (3×30 mL), dried over anhydrous Na₂SO₄ and concentrated. The crude product was purified by Prep-HPLC with following conditions: Column, welch Vltimate XB-C18, 50×250 mm, 10 μm; Mobile Phase: 35-750 MeCN/0.1% aqueous formic acid; to afford 6-[3-(5-chloro-2-methoxypyridine-3-sulfonamido)-2,6-difluorophenyl]-N-(cyclopropylmethyl)imidazo[1,5-a]pyrazine-1-carboxamide (75 mg, 34% yield) as a white solid.

LCMS (ES, m/z): [M+H]⁺: 549

¹H NMR (300 MHz, DMSO-d₆) δ 10.45 (s, 1H), 9.52 (d, J=1.6 Hz, 1H), 8.68 (d, J=3.4 Hz, 2H), 8.55-8.29 (m, 2H), 8.09 (d, J=2.6 Hz, 1H), 7.42 (td, J=8.8, 5.8 Hz, 1H), 7.26 (td, J 8.7, 1.4 Hz, 1H), 3.92 (s, 3H), 3.24-3.06 (m, 2H), 1.09 (ddd, J=12.6, 7.8, 5.1 Hz, 1H), 0.55-0.35 (m, 2H), 0.35-0.19 (m, 2H).

### Example 182: Synthesis of 6-[3-(5-chloro-2-ethoxypyridine-3-sulfonamido)-2,6-difluorophenyl]-N-methylimidazo[1,5-a]pyrazine-1-carboxamide (Compound 167)

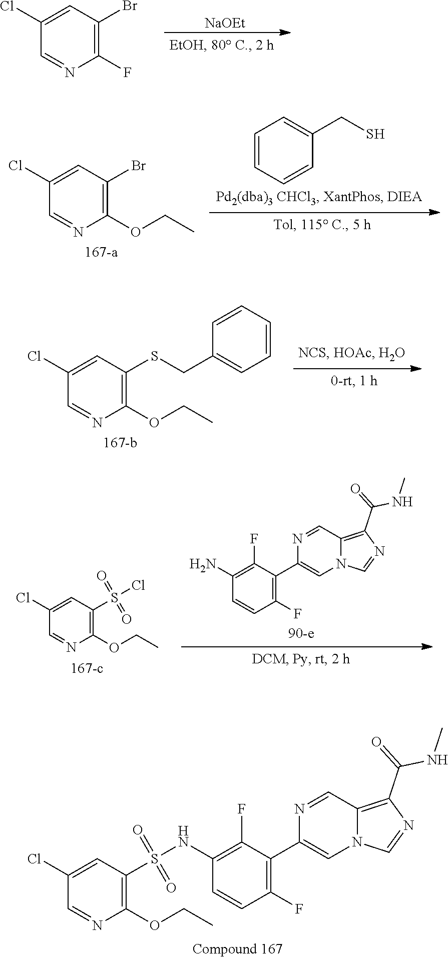

### Synthesis of 167-a: 3-bromo-5-chloro-2-ethoxvpyridine

A solution of 3-bromo-5-chloro-2-fluoropyridine (1 g, 4.8 mmol, 1 equiv) and sodium ethoxide (0.97 g, 14 mmol, 3 equiv) in EtOH (10 mL) was stirred for 2 h at 80° C. The mixture was cooled and quenched with H₂O (100 mL), then extracted with EA (3×50 mL). The combined organics were washed with brine (100 mL), dried over anhydrous Na₂SO₄ and concentrated. The residue was purified by Flash-Prep-HPLC with the following conditions: Column, WelFlash™ C18-I, Spherical C18 20-40 m, 120 g; mobile phase: 40-90% MeCN/0.1% aqueous formic acid; to afford 3-bromo-5-chloro-2-ethoxypyridine (0.7 g, 62% yield) as a light yellow oil.

LCMS (ES, m/z): [M+H]⁺: 236, 238

### Synthesis of 167-b: 3-(benzylsulfanyl)-5-chloro-2-ethoxypyridine

To a stirred solution of 3-bromo-5-chloro-2-ethoxypyridine (645 mg, 2.7 mmol, 1 equiv), DIEA (1058 mg, 8.2 mmol, 3 equiv) and benzyl mercaptan (508 mg, 4.1 mmol, 1.5 equiv) in toluene (10 mL) were added XantPhos (316 mg, 0.54 mmol, 0.2 equiv) and Pd₂(dba)₃CHCl₃ (282 mg, 0.27 mmol, 0.1 equiv) in portions under nitrogen atmosphere. The resulting mixture was stirred for 5 h at 115° C., then cooled, quenched with H₂O (20 mL) and extracted with EA (3×10 mL). The combined organics were washed with brine (20 mL), dried over anhydrous Na₂SO₄ and concentrated. The residue was purified by Flash-Prep-HPLC with the following conditions: Column, WelFlash™ C18-I, Spherical C18 20-40 m, 120 g; mobile phase: 50-90% MeCN/0.1% aqueous formic acid; to afford 3-(benzylsulfanyl)-5-chloro-2-ethoxypyridine (470 mg, 62% yield) as a white solid.

LCMS (ES, m/z): [M+H]⁺: 280

### Synthesis of 167-c: 5-chloro-2-ethoxypyridine-3-sulfonyl chloride

To a stirred solution of 3-(benzylsulfanyl)-5-chloro-2-ethoxypyridine (476 mg, 1.7 mmol, 1 equiv) in HOAc (6 mL) and H₂O (2 mL) was added NCS (795 mg, 5.95 mmol, 3.5 equiv) in portions at 0° C. The reaction was stirred for 0.5 h at room temperature, then quenched with H₂O (10 mL) and extracted with EA (3×5 mL). The combined organics were washed with NaHCO₃ (10 mL) and brine (10 mL), dried over anhydrous Na₂SO₄ and concentrated to give crude 5-chloro-2-ethoxypyridine-3-sulfonyl chloride (640 mg) as a yellow oil, which was used in the next step directly without further purification.

LCMS (ES, m/z): [M+H]⁺: 256

### Synthesis of Compound 167: 6-[3-(5-chloro-2-ethoxypyridine-3-sulfonamido)-2,6-difluorophenyl]-N-methylimidazo[1,5-a]pyrazine-1-carboxamide

To a stirred solution of 5-chloro-2-ethoxypyridine-3-sulfonyl chloride (200 mg, 0.78 mmol, 1 equiv) in pyridine (2 mL) was added 6-(3-amino-2,6-difluorophenyl)-N-methylimidazo[1,5-a]pyrazine-1-carboxamide (474 mg, 1.56 mmol, 2 equiv) in DCM (0.5 mL) dropwise at room temperature. The resulting mixture was stirred for 1 h then quenched with H₂O (10 mL) and extracted with EA (3×5 mL). The combined organics were washed with brine (10 mL), dried over anhydrous Na₂SO₄ and concentrated. The residue was purified by prep-HPLC with the following conditions: Column, welch Vltimate XB-C18, 50×250 mm, 10 μm; Mobile Phase: 35-70% MeCN/0.1% aqueous formic acid; to afford 6-[3-(5-chloro-2-ethoxypyridine-3-sulfonamido)-2,6-difluorophenyl]-N-methylimidazo[1,5-a]pyrazine-1-carboxamide (150 mg, 37% yield) as a white solid.

LCMS (ES, m/z): [M+H]⁺: 523

¹H NMR (300 MHz, DMSO-d₆) δ 10.34 (s, 1H), 9.51 (d, J=1.7 Hz, 1H), 8.65 (d, J=1.9 Hz, 2H), 8.44 (dd, J=18.6, 3.6 Hz, 2H), 8.09 (d, J=2.6 Hz, 1H), 7.43 (td, J=9.0, 5.9 Hz, 1H), 7.24 (td, J=9.2, 1.7 Hz, 1H), 4.39 (q, J=7.0 Hz, 2H), 2.84 (d, J=4.8 Hz, 3H), 1.22 (t, J=7.0 Hz, 3H).

### Example 183: Synthesis of 6-[3-(5-chloro-2-isopropoxypyridine-3-sulfonamido)-2,6-difluorophenyl]-N-methylimidazo[1,5-a]pyrazine-1-carboxamide (Compound 168)

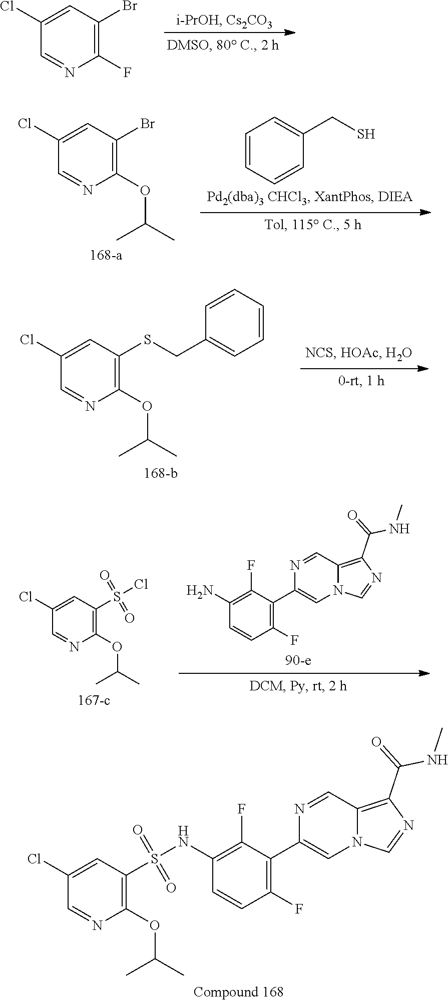

### Synthesis of 168-a: 3-bromo-5-chloro-2-isopropoxypyridine

A solution of 3-bromo-5-chloro-2-fluoropyridine (1 g, 4.8 mmol, 1 equiv), i-PrOH (0.86 g, 14 mmol, 3 equiv) and Cs₂CO₃ (4.65 g, 14 mmol, 3 equiv) in DMSO (10 mL) was stirred for 2 h at 80° C. The resulting mixture was quenched with H₂O (20 mL) and extracted with EA (3×10 mL). The combined organics were washed with brine (20 mL), dried over anhydrous Na₂SO₄ and concentrated. The residue was purified by Flash-Prep-HPLC with the following conditions: Column, WelFlash™ C18-I, Spherical C18 20-40 m, 120 g; mobile phase: 50-90% MeCN/0.1% aqueous formic acid; to afford 3-bromo-5-chloro-2-isopropoxypyridine (800 mg, 67% yield) as a yellow solid.

LCMS (ES, m/z): [M+H]⁺: 250, 252

### Synthesis of 168-b: 3-(benzylsulfanyl)-5-chloro-2-isopropoxypyridine

To a stirred solution of 3-bromo-5-chloro-2-isopropoxypyridine (775 mg, 3.1 mmol, 1 equiv), DIEA (800 mg, 6.2 mmol, 2 equiv) and benzyl mercaptan (576 mg, 4.6 mmol, 1.5 equiv) in toluene (10 mL) were added XantPhos (358 mg, 0.62 mmol, 0.2 equiv) and Pd₂(dba)₃CHCl₃ (320 mg, 0.31 mmol, 0.1 equiv) in portions at room temperature under nitrogen atmosphere. The reaction was stirred for 2 h at 80° C. then quenched with H₂O (20 mL) and extracted with EA (3×10 mL). The combined organics were washed with brine (20 mL), dried over anhydrous Na₂SO₄ and concentrated. The residue was purified by Flash-Prep-HPLC with the following conditions: Column, WelFlash™ C18-I, Spherical C18 20-m, 120 g; mobile phase: 50-95% MeCN/0.1% aqueous formic acid; to afford 3-(benzylsulfanyl)-5-chloro-2-isopropoxypyridine (570 mg, 63% yield) as a light yellow oil.

LCMS (ES, m/z): [M+H]⁺: 294

### Synthesis of 168-c: 5-chloro-2-isopropoxypyridine-3-sulfonyl chloride

To a stirred solution of 3-(benzylsulfanyl)-5-chloro-2-isopropoxypyridine (570 mg, 1.9 mmol, 1 equiv) in HOAc (6 mL) and H₂O (2 mL) was added NCS (907 mg, 6.8 mmol, 3.5 equiv) in portions at 0° C. The reaction was stirred for 0.5 h at room temperature, then quenched with H₂O (20 mL) and extracted with EA (3×10 mL). The combined organics were washed with NaHCO₃ (20 mL) and brine (20 mL), dried over anhydrous Na₂SO₄ and concentrated to give crude 5-chloro-2-isopropoxypyridine-3-sulfonyl chloride (730 mg) as a yellow oil which was used in next step directly without further purification.

LCMS (ES, m/z): [M+H]⁺: 270

### Synthesis of Compound 168: 6-[3-(5-chloro-2-isopropoxypyridine-3-sulfonamido)-2,6-difluorophenyl]-N-methylimidazo[1,5-a]pyrazine-1-carboxamide

To a stirred solution of 6-(3-amino-2,6-difluorophenyl)-N-methylimidazo[1,5-a]pyrazine-1-carboxamide (197 mg, 0.65 mmol, 0.5 equiv) in pyridine (4 mL) was added 5-chloro-2-isopropoxypyridine-3-sulfonyl chloride (350 mg, 1.3 mmol, 1 equiv) in DCM (0.5 mL) dropwise. The resulting mixture was stirred for 0.5 h then concentrated. The residue was purified by prep-HPLC with the following conditions: Column, welch Vltimate XB-C18, 50×250 mm, 10 μm; Mobile Phase: 35-70% MeCN/0.1% aqueous formic acid; to afford 6-[3-(5-chloro-2-isopropoxypyridine-3-sulfonamido)-2,6-difluorophenyl]-N-methylimidazo[1,5-a]pyrazine-1-carboxamide (150 mg, 22% yield) as a white solid.

LCMS (ES, m/z): [M+H]⁺: 537

¹H NMR (300 MHz, DMSO-d₆) δ 10.24 (s, 1H), 9.51 (d, J=1.6 Hz, 1H), 8.66 (d, J=1.5 Hz, 2H), 8.47 (d, J=2.6 Hz, 1H), 8.41 (q, J=4.5 Hz, 1H), 8.10 (d, J=2.6 Hz, 1H), 7.45 (td, J=8.9, 5.9 Hz, 1H), 7.26 (td, J=9.1, 1.7 Hz, 1H), 5.31 (h, J=6.0 Hz, 1H), 2.84 (d, J=4.7 Hz, 3H), 1.21 (d, J=6.1 Hz, 6H).

### Example 184: Synthesis of 6-[3-(5-chloro-2-cyclopropoxypyridine-3-sulfonamido)-2,6-difluorophenyl]-N-methylimidazo[1,5-a]pyrazine-1-carboxamide (Compound 169)

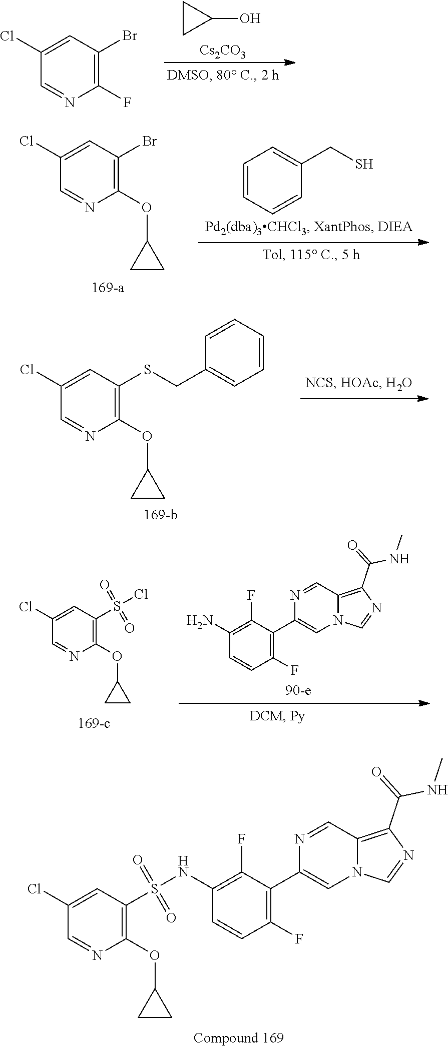

### Synthesis of 169-a 3-bromo-5-chloro-2-cyclopropoxypyridine

A solution of 3-bromo-5-chloro-2-fluoropyridine (1 g, 4.8 mmol, 1 equiv), caesium carbonate (4.7 g, 14 mmol, 3 equiv) and cyclopropanol (0.83 g, 14 mmol, 3 equiv) in DMSO (10 mL) was stirred for 2 h at 80° C. The resulting mixture was quenched with H₂O (20 mL) and extracted with EA (3×10 mL). The combined organics were washed with brine (20 mL), dried over anhydrous Na₂SO₄ and concentrated. The residue was purified by Flash-Prep-HPLC with the following conditions: Column, WelFlash™ C18-I, Spherical C18 20-40 m, 120 g; mobile phase: 40-90% MeCN/0.1% aqueous formic acid; to afford 3-bromo-5-chloro-2-cyclopropoxypyridine (800 mg, 68% yield) as a light yellow solid.

LCMS (ES, m/z): [M+H]⁺: 248, 250

### Synthesis of 169-b: 3-(benzylsulfanyl)-5-chloro-2-cyclopropoxypyridine

To a stirred solution of 3-bromo-5-chloro-2-cyclopropoxypyridine (775 mg, 3.1 mmol, 1 equiv), DIEA (806 mg, 6.2 mmol, 2 equiv) and benzyl mercaptan (581 mg, 4.7 mmol, 1.5 equiv) in toluene (10 mL) were added XantPhos (361 mg, 0.62 mmol, 0.2 equiv) and Pd₂(dba)₃CHCl₃ (323 mg, 0.31 mmol, 0.1 equiv) in portions at room temperature under nitrogen atmosphere. The resulting mixture was stirred for 2 h at 115° C. then cooled and quenched with H₂O (30 mL) and extracted with EA (3×10 mL). The combined organics were washed with brine (20 mL), dried over anhydrous Na₂SO₄ and concentrated. The residue was purified by Flash-Prep-HPLC with the following conditions: Column, WelFlash™ C18-I, Spherical C18 20-40 m, 120 g; mobile phase: 50-80% MeCN/0.1% aqueous formic acid; to afford 3-(benzylsulfanyl)-5-chloro-2-cyclopropoxypyridine (565 mg, 62% yield) as a light yellow solid.

LCMS (ES, m/z): [M+H]⁺: 292

### Synthesis of 169-c: 5-chloro-2-cyclopropoxypyridine-3-sulfonyl chloride

To a stirred solution of 3-(benzylsulfanyl)-5-chloro-2-cyclopropoxypyridine (560 mg, 1.9 mmol, 1 equiv) in HOAc (6 mL) and H₂O (2 mL) was added NCS (897 mg, 6.7 mmol, 3.5 equiv) in portions at 0° C. The resulting mixture was stirred for 30 min at room temperature, then quenched with H₂O (20 mL) and extracted with EA (3×10 mL). The combined organics were washed with NaHCO₃ (30 mL), dried over anhydrous Na₂SO₄ and concentrated to afford 5-chloro-2-cyclopropoxypyridine-3-sulfonyl chloride (717 mg) as a yellow oil. The crude product mixture was used in the next step directly without further purification.

LCMS (ES, m/z): [M+H]⁺: 268

### Synthesis of Compound 169: 6-[3-(5-chloro-2-cyclopropoxypyridine-3-sulfonamido)-2,6-difluorophenyl]-N-methylimidazo[1,5-a]pyrazine-1-carboxamide

To a stirred solution of 6-(3-amino-2,6-difluorophenyl)-N-methylimidazo[1,5-a]pyrazine-1-carboxamide (167 mg, 0.56 mmol, 0.5 equiv) in pyridine (10 mL) was added 5-chloro-2-cyclopropoxypyridine-3-sulfonyl chloride (300 mg, 1.1 mmol, 1 equiv) in DCM (0.5 mL) dropwise. The resulting mixture was stirred for 1 h then concentrated. The residue was purified by prep-HPLC with the following conditions: Column, welch Vltimate XB-C18, 50×250 mm, 10 μm; Mobile Phase: 30-70% MeCN/0.1% aqueous formic acid; to afford 6-[3-(5-chloro-2-cyclopropoxypyridine-3-sulfonamido)-2,6-difluorophenyl]-N-methylimidazo[1,5-a]pyrazine-1-carboxamide (110 mg, 18% yield) as a white solid.

LCMS (ES, m/z): [M+H]⁺: 535

¹H NMR (300 MHz, DMSO-d₆) δ 10.33 (s, 1H), 9.51 (d, J=1.7 Hz, 1H), 8.65 (d, J=2.9 Hz, 2H), 8.53 (d, J=2.5 Hz, 1H), 8.41 (d, J=4.9 Hz, 1H), 8.10 (d, J=2.6 Hz, 1H), 7.42 (td, J=8.9, 5.8 Hz, 1H), 7.33-7.19 (m, 1H), 4.32 (tt, J=6.2, 3.1 Hz, 1H), 2.84 (d, J=4.8 Hz, 3H), 0.81-0.52 (m, 4H).

### Example 185: Synthesis of 6-[2,6-difluoro-3-(5-fluoro-2-methoxypyridine-3-sulfonamido)phenyl]-5-fluoro-N-methylimidazo[1,5-a]pyrazine-1-carboxamide (Compound 170)

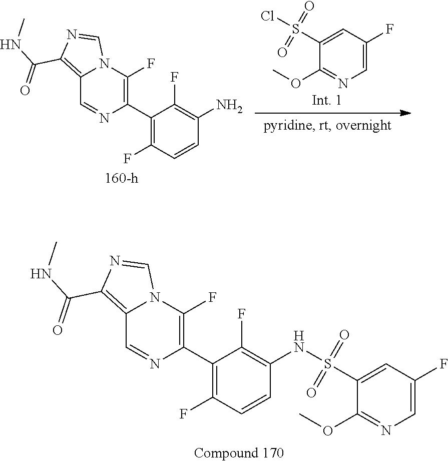 [SMILES: CNC(=O)c1ncn2c(F)c(-c3c(F)ccc(N)c3F)ncc12 ; COc1ncc(F)cc1S(=O)(=O)Cl ; CNC(=O)c1ncn2c(F)c(-c3c(F)ccc(NS(=O)(=O)c4cc(F)cnc4OC)c3F)ncc12]

To a stirred solution of 6-(3-amino-2,6-difluorophenyl)-5-fluoro-N-methylimidazo[1,5-a]pyrazine-1-carboxamide (60 mg, 0.2 mmol, 1 equiv) in pyridine (3 mL) was added 5-fluoro-2-methoxypyridine-3-sulfonyl chloride (84 mg, 0.4 mmol, 2 equiv). The reaction was stirred overnight then concentrated. The residue was purified by prep-HPLC with the following conditions: Column, welch Vltimate XB-C18, 50×250 mm, 10 m; Mobile Phase: 17-55% MeCN/0.1% aqueous formic acid; to give 6-[2,6-difluoro-3-(5-fluoro-2-methoxypyridine-3-sulfonamido)phenyl]-5-fluoro-N-methylimidazo[1,5-a]pyrazine-1-carboxamide (20 mg, 21% yield) as an off-white solid.

LCMS (ES, m/z): [M+H]⁺: 511

¹H NMR (300 MHz, DMSO-d₆) δ 10.48 (s, 1H), 9.36 (d, J=2.8 Hz, 1H), 8.92 (s, 1H), 8.55 (d, J=4.9 Hz, 1H), 8.46 (d, J=3.0 Hz, 1H), 8.02 (dd, J=7.3, 3.0 Hz, 1H), 7.51 (td, J=8.9, 5.8 Hz, 1H), 7.29 (dd, J=9.8, 8.3 Hz, 1H), 3.89 (s, 3H), 2.85 (d, J=4.7 Hz, 3H).

### Example 186: Synthesis of 6-[3-(5-chloro-2-methoxypyridine-3-sulfonamido)-2,6-difluorophenyl]imidazo[1,5-a]pyrazine-1-carboxamide (Compound 171)

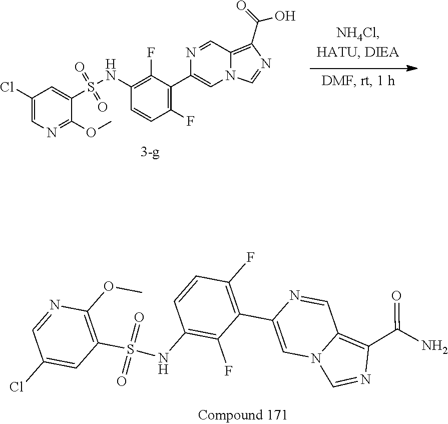 [SMILES: COc1ncc(Cl)cc1S(=O)(=O)Nc1ccc(F)c(-c2cn3cnc(C(=O)O)c3cn2)c1F ; COc1ncc(Cl)cc1S(=O)(=O)Nc1ccc(F)c(-c2cn3cnc(C(N)=O)c3cn2)c1F]

### Synthesis of Compound 171: 6-[3-(5-chloro-2-methoxypyridine-3-sulfonamido)-2,6-difluorophenyl]imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added NH₄Cl (32 mg, 0.6 mmol, 1.5 equiv) and HATU (230 mg, 0.6 mmol, 1.5 equiv). The reaction was stirred for 1 then diluted with H₂O (50 mL). The resulting mixture was extracted with EA (3×50 mL), and the combined organics were washed with brine (3×30 mL), dried over anhydrous Na₂SO₄, then concentrated. The crude product was purified by Prep-HPLC with following conditions: Column: XBridge Shield RP18 OBD, 5 μm, 1 9*150 mm; Mobile Phase: 4-27% MeCN/0.05% aqueous ammonia; to afford 6-[3-(5-chloro-2-methoxypyridine-3-sulfonamido)-2,6-difluorophenyl]imidazo[1,5-a]pyrazine-1-carboxamide (60 mg, 30% yield) as a white solid.

LCMS (ES, m/z): [M+H]⁺: 495

¹H NMR (300 MHz, DMSO-d₆) δ 10.44 (s, 1H), 9.50 (d, J=1.6 Hz, 1H), 8.71-8.62 (m, 2H), 8.51 (d, J=2.6 Hz, 1H), 8.09 (d, J=2.6 Hz, 1H), 7.79 (s, 1H), 7.51 (s, 1H), 7.42 (td, J=8.8, 5.8 Hz, 1H), 7.25 (td, J=9.1, 1.6 Hz, 1H), 3.92 (s, 3H).

### Example 187: Synthesis of 6-[3-(5-chloro-2-methoxypyridine-3-sulfonamido)-2,6-difluorophenyl]-N-[3-(dimethylamino)propyl]imidazo[1,5-a]pyrazine-1-carboxamide (Compound 172)

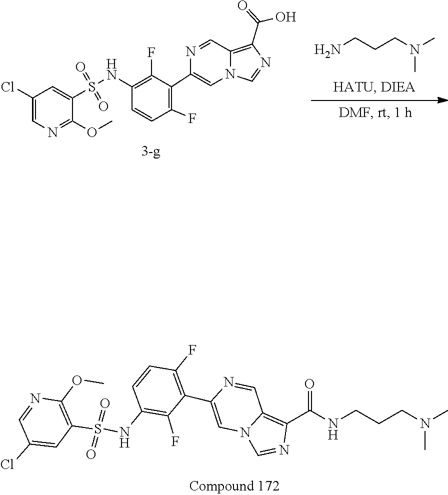

### Synthesis of Compound 172: 6-[3-(5-chloro-2-methoxypyridine-3-sulfonamido)-2,6-difluorophenyl]-N-[3-(dimethylamino)propyl]imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added dimethylaminopropylamine (61 mg, 0.6 mmol, 1.5 equiv) and HATU (230 mg, 0.6 mmol, 1.5 equiv). The reaction was stirred for 1 h, then diluted with H₂O (50 mL). The resulting mixture was extracted with EA (3×50 mL), and the combined organics were washed with brine (3×30 mL), dried over anhydrous Na₂SO₄, then concentrated. The crude product was purified by Prep-HPLC with following conditions: Column, XBridge Prep C18 OBD, 5 μm, 19*150 mm, Mobile Phase: 12-34% MeCN/0.5% aqueous ammonia; to afford 6-[3-(5-chloro-2-methoxypyridine-3-sulfonamido)-2,6-difluorophenyl]-N-[3-(dimethylamino)propyl]imidazo[1,5-a]pyrazine-1-carboxamide (46 mg, 20% yield) as a white solid.

LCMS (ES, m/z): [M+H]⁺: 580

¹H NMR (300 MHz, DMSO-d₆) δ 9.52 (d, J=1.6 Hz, 1H), 8.65 (d, J=2.5 Hz, 2H), 8.60 (t, J=6.0 Hz, 1H), 8.35 (d, J=2.7 Hz, 1H), 8.04 (d, J=2.7 Hz, 1H), 7.32 (td, J=9.1, 6.0 Hz, 1H), 7.05 (t, J=9.3 Hz, 1H), 3.86 (s, 3H), 3.37 (d, J=6.5 Hz, 2H), 2.67 (t, J=7.2 Hz, 2H), 2.45 (s, 6H), 1.80 (m, 2H).

### Example 188: Synthesis of (R)-6-(3-((5-chloro-2-methoxypyridine)-3-sulfonamido)-2,6-difluorophenyl)-N-(1-methylpyrrolidin-3-yl)imidazo[1,5-a]pyrazine-1-carboxamide & and (S)-6-(3-((5-chloro-2-methoxypyridine)-3-sulfonamido)-2,6-difluorophenyl)-N-(1-methylpyrrolidin-3-yl)imidazo[1,5-a]pyrazine-1-carboxamide (Compounds 173-1 & 173-2)

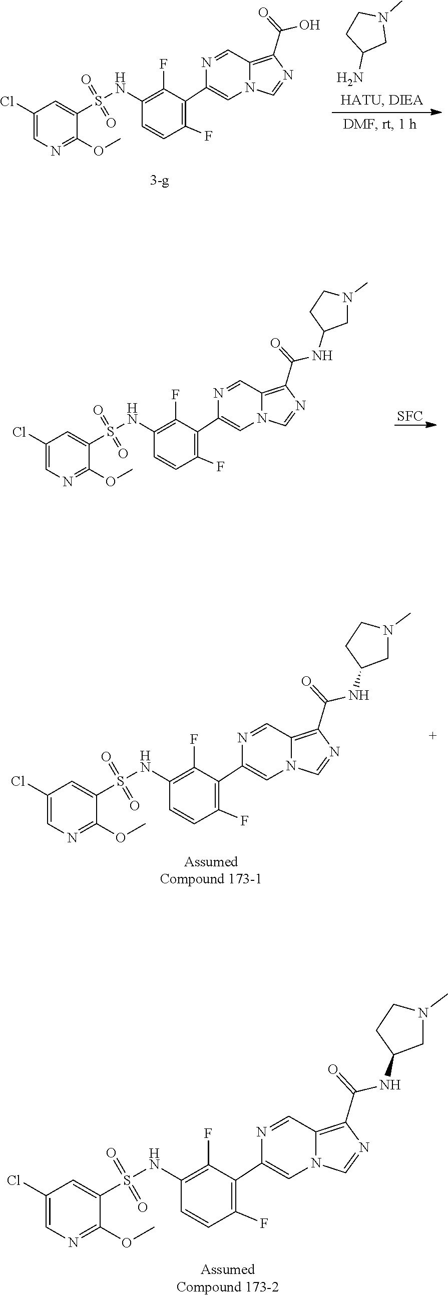

### Synthesis of Compounds 173-1 & 173-2: (R)-6-(3-((5-chloro-2-methoxypyridine)-3-sulfonamido)-2,6-difluorophenyl)-N-(1-methylpyrrolidin-3-yl)imidazo[1,5-a]pyrazine-1-carboxamide and (S)-6-(3-((5-chloro-2-methoxypyridine)-3-sulfonamido)-2,6-difluorophenyl)-N-(1-methylpyrrolidin-3-yl)imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added 1-methylpyrrolidin-3-amine (61 mg, 0.6 mmol, 1.5 equiv) and HATU (230 mg, 0.6 mmol, 1.5 equiv. The reaction was stirred for 1 h at room temperature, then diluted with H₂O (50 mL). The resulting mixture was extracted with EA (3×50 mL), and the combined organics were washed with brine (3×30 mL), dried over anhydrous Na₂SO₄ and concentrated. The crude product was purified by Prep-HPLC with following conditions: Column: welch Vltimate XB-C18, 50×250 mm, 10 μm; Mobile Phase: 5-30% MeCN/0.1% aqueous formic acid; to afford racemic 6-(3-((5-chloro-2-methoxypyridine)-3-sulfonamido)-2,6-difluorophenyl)-N-(1-methylpyrrolidin-3-yl)imidazo[1,5-a]pyrazine-1-carboxamide (150 mg, 64% yield) as a white solid.

This was separated by SFC with the following conditions Column: Lux 5 μm Cellulose-2, 3*25 cm, 5 μm; Mobile Phase: 55% 1:1 MeOH:MeCN/CO₂; Flow rate: 100 mL/min; Temperature: 35° C.; to give (R)-6-(3-((5-chloro-2-methoxypyridine)-3-sulfonamido)-2,6-difluorophenyl)-N-(1-methylpyrrolidin-3-yl)imidazo[1,5-a]pyrazine-1-carboxamide (retention time 9.65 mins, 60 mg, stereochemistry randomly assigned) and (S)-6-(3-((5-chloro-2-methoxypyridine)-3-sulfonamido)-2,6-difluorophenyl)-N-(1-methylpyrrolidin-3-yl)imidazo[1,5-a]pyrazine-1-carboxamide (retention time 15.59 mins, 65 mg, stereochemistry assigned as opposite to other enantiomer) as white solids.

LCMS (ES, m/z): [M+H]⁺: 578

¹H NMR (300 MHz, DMSO-d₆) δ 9.49 (d, J=1.6 Hz, 1H), 8.64 (d, J=7.2 Hz, 2H), 8.30 (d, J=2.6 Hz, 1H), 8.01 (d, J=2.6 Hz, 1H), 7.29 (td, J=9.0, 5.9 Hz, 1H), 7.02 (t, J=9.2 Hz, 1H), 4.59 (t, J=8.0 Hz, 1H), 3.83 (s, 3H), 3.12 (q, J=9.9, 9.5 Hz, 2H), 2.97-2.79 (m, 2H), 2.56 (s, 3H), 2.31 (dd, J=13.9, 6.0 Hz, 1H), 1.94 (q, J=6.5 Hz, 1H).

LCMS (ES, m/z): [M+H]⁺: 578

¹H NMR (300 MHz, DMSO-d₆) δ 9.49 (d, J=1.7 Hz, 1H), 8.64 (d, J=6.6 Hz, 2H), 8.30 (d, J=2.6 Hz, 1H), 8.01 (d, J=2.6 Hz, 1H), 7.32-7.16 (m, 1H), 7.02 (t, J=8.9 Hz, 1H), 4.56 (d, J=14.5 Hz, 1H), 3.83 (s, 3H), 3.20-3.00 (m, 2H), 2.97-2.72 (m, 2H), 2.55 (s, 3H), 2.30 (td, J=14.2, 8.5 Hz, 1H), 1.95 (dt, J=12.4, 6.3 Hz, 1H).

### Example 189: Synthesis of 6-[3-(5-chloro-2-methoxypyridine-3-sulfonamido)-2,6-difluorophenyl]-N-(1,2-oxazol-4-yl)imidazo[1,5-a]pyrazine-1-carboxamide (Compound 174)

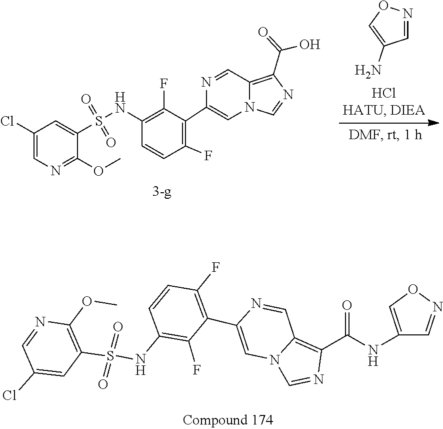 [SMILES: COc1ncc(Cl)cc1S(=O)(=O)Nc1ccc(F)c(-c2cn3cnc(C(=O)O)c3cn2)c1F ; Nc1cnoc1 ; COc1ncc(Cl)cc1S(=O)(=O)Nc1ccc(F)c(-c2cn3cnc(C(=O)Nc4cnoc4)c3cn2)c1F]

### Synthesis of Compound 174: 6-[3-(5-chloro-2-methoxypyridine-3-sulfonamido)-2,6-difluorophenyl]-N-(1,2-oxazol-4-yl)imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added 1,2-oxazol-4-amine (50 mg, 0.6 mmol, 1.5 equiv) and HATU (230 mg, 0.6 mmol, 1.5 equiv). The reaction was stirred for 1 h at room temperature, then diluted with H₂O (50 mL). The resulting mixture was extracted with EA (3×50 mL), and the combined organics were washed with brine (3×30 mL), dried over anhydrous Na₂SO₄ and concentrated. The crude product was purified by Prep-HPLC with following conditions: Column: XBridge Shield RP18 OBD, 5 μm, 19*150 mm; Mobile Phase: 14-27% MeCN/0.05% aqueous ammonia; to afford 6-[3-(5-chloro-2-methoxypyridine-3-sulfonamido)-2,6-difluorophenyl]-N-(1,2-oxazol-4-yl)imidazo[1,5-a]pyrazine-1-carboxamide (73 mg, 32% yield) as a white solid.

LCMS (ES, m/z): [M+H]⁺: 562

### Example 190: Synthesis of 6-[3-(5-chloro-2-methoxypyridine-3-sulfonamido)-2,6-difluorophenyl]-N-[2-(morpholin-4-yl)ethyl]imidazo[1,5-a]pyrazine-1-carboxamide (Compound 175)

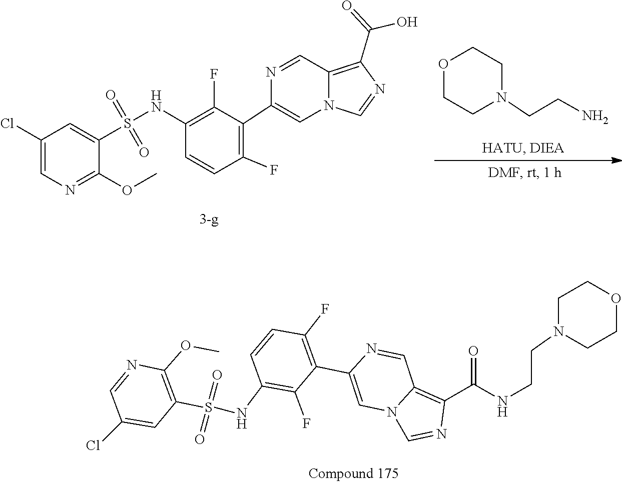 [SMILES: COc1ncc(Cl)cc1S(=O)(=O)Nc1ccc(F)c(-c2cn3cnc(C(=O)O)c3cn2)c1F ; NCCN1CCOCC1 ; COc1ncc(Cl)cc1S(=O)(=O)Nc1ccc(F)c(-c2cn3cnc(C(=O)NCCN4CCOCC4)c3cn2)c1F]

### Synthesis of Compound 175: 6-[3-(5-chloro-2-methoxypyridine-3-sulfonamido)-2,6-difluorophenyl]-N-[2-(morpholin-4-yl)ethyl]imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added N-aminoethylmorpholine (78 mg, 0.6 mmol, 1.5 equiv) and HATU (230 mg, 0.6 mmol, 1.5 equiv). The reaction was stirred for 1 h at room temperature, then was diluted with H₂O (50 mL). The resulting mixture was extracted with EA (3×50 mL) and the combined organics were washed with brine (3×30 mL), dried over anhydrous Na₂SO₄ and concentrated. The crude product was purified by Prep-HPLC with following conditions: Column: Sunfire Prep C18 OBD, 50*250 mm 5 μm 10 nm; Mobile Phase: 5-40% MeCN/0.05% aqueous ammonia; to afford 6-[3-(5-chloro-2-methoxypyridine-3-sulfonamido)-2,6-difluorophenyl]-N-[2-(morpholin-4-yl)ethyl]imidazo[1,5-a]pyrazine-1-carboxamide (54 mg, 22% yield) as a white solid.

LCMS (ES, m/z): [M+H]⁺: 608

¹H NMR (300 MHz, DMSO-d₆) δ 10.42 (s, 1H), 9.51 (d, J=1.6 Hz, 1H), 8.67 (d, J=3.8 Hz, 2H), 8.48 (d, J=2.6 Hz, 1H), 8.31 (t, J=5.9 Hz, 1H), 8.08 (d, J=2.6 Hz, 1H), 7.40 (td, J=8.9, 5.8 Hz, 1H), 7.22 (td, J=9.1, 1.6 Hz, 1H), 3.91 (s, 3H), 3.59 (t, J=4.6 Hz, 4H), 3.46 (q, J=6.4 Hz, 2H), 2.54 (d, J=7.0 Hz, 2H), 2.46 (t, J=4.7 Hz, 4H).

### Example 191: Synthesis of 6-[3-[5-chloro-2-(2-methylpropoxy)pyridine-3-sulfonamido]-2,6-difluorophenyl]-N-methylimidazo[1,5-a]pyrazine-1-carboxamide (Compound 176)

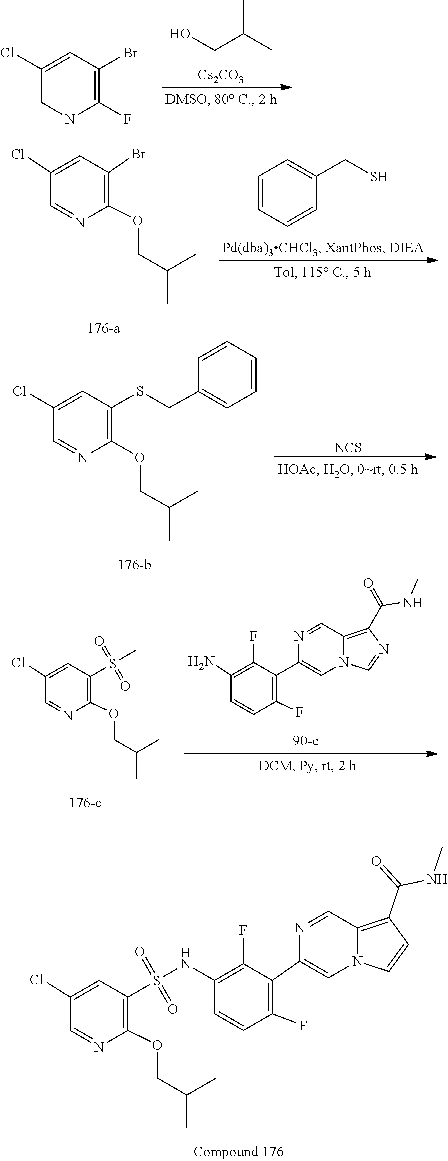

### Synthesis of 176-a: 3-bromo-5-chloro-2-(2-methylpropoxy)pyridine

To a stirred solution of 3-bromo-5-chloro-2-fluoropyridine (1 g, 4.8 mmol, 1 equiv) and cesium carbonate (4.7 g, 14 mmol, 3 equiv) in DMSO (10 mL) was added isobutanol (1.06 g, 14 mmol, 3 equiv). The resulting mixture was stirred for 2 h at 80° C., then cooled and quenched with H₂O (20 mL). The mixture was extracted with EtOAc (3×10 mL), and the combined organics washed with brine (10 mL), dried over anhydrous Na₂SO₄ and concentrated. The residue was purified by Flash-Prep-HPLC with the following conditions: Column, WelFlash™ C18-I, Spherical C18 20-40 m, 120 g; mobile phase: 50-90% MeCN/0.1% aqueous formic acid; to afford 3-bromo-5-chloro-2-(2-methylpropoxy)pyridine (1.07 g, 85% yield) as a yellow solid.

LCMS (ES, m/z): [M+H]⁺: 264, 266

### Synthesis of 176-b: 3-(benzylsulfanyl)-5-chloro-2-(2-methylpropoxy)pyridine

To a stirred solution of 3-bromo-5-chloro-2-(2-methylpropoxy)pyridine (1.08 g, 4.1 mmol, 1 equiv), DIEA (1.06 g, 8.2 mmol, 2 equiv) and benzyl mercaptan (0.76 g, 6.1 mmol, 1.5 equiv) in toluene (15 mL) were added XantPhos (0.47 g, 0.82 mmol, 0.2 equiv) and Pd₂(dba)₃·CHCl₃ (0.42 g, 0.41 mmol, 0.1 equiv) in portions at room temperature under nitrogen atmosphere. The resulting mixture was stirred for 5 h at 115° C. then cooled and quenched with H₂O (20 mL). The mixture was extracted with EtOAc (3×10 mL), and the combined organics were washed with brine (20 mL), dried over anhydrous Na₂SO₄ and concentrated. The residue was purified by Flash-Prep-HPLC with the following conditions: Column, WelFlash™ C18-I, Spherical C18 20-40 m, 120 g; mobile phase: 40-90% MeCN/0.1% aqueous formic acid; to afford 3-(benzylsulfanyl)-5-chloro-2-(2-methylpropoxy)pyridine (1.16 g, 92% yield) as a white solid.

LCMS (ES, m/z): [M+H]⁺: 308

### Synthesis of 176-c: 5-chloro-2-(2-methylpropoxy)pyridine-3-sulfonyl chloride

To a stirred solution of 3-(benzylsulfanyl)-5-chloro-2-(2-methylpropoxy)pyridine (710 mg, 2.3 mmol, 1 equiv) in HOAc (9 mL) and H₂O (3 mL) was added NCS (1080 mg, 8.1 mmol, 3.5 equiv) in portions at 0° C. The resulting mixture was stirred for 1 h at room temperature, then concentrated to afford crude 5-chloro-2-(2-methylpropoxy)pyridine-3-sulfonyl chloride (620 mg, 95% yield) as a white oil which was used in next step directly without further purification.

### Synthesis of Compound 176: 6-[3-[5-chloro-2-(2-methylpropoxy)pyridine-3-sulfonamido]-2,6-difluorophenyl]-N-methylimidazo[1,5-a]pyrazine-1-carboxamide

To a stirred solution of 6-(3-amino-2,6-difluorophenyl)-N-methylimidazo[1,5-a]pyrazine-1-carboxamide (192 mg, 0.63 mmol, 0.3 equiv) in pyridine (5 mL) was added 5-chloro-2-(2-methylpropoxy)pyridine-3-sulfonyl chloride (600 mg, 2.1 mmol, 1 equiv) in DCM (0.5 mL). The resulting mixture was stirred for 2 h at room temperature, then quenched with H₂O (25 mL) and extracted with EtOAc (3×10 mL). The combined organics were washed with brine (20 mL), dried over anhydrous Na₂SO₄ and concentrated. The residue was purified by prep-HPLC with the following conditions: Column, welch Vltimate XB-C18, 50×250 mm, 10 μm; Mobile Phase: 20-65% MeCN/0.1% aqueous formic acid; to afford 6-[3-[5-chloro-2-(2-methylpropoxy)pyridine-3-sulfonamido]-2,6-difluorophenyl]-N-methylimidazo[1,5-a]pyrazine-1-carboxamide (120 mg, 10% yield) as a white solid.

LCMS (ES, m/z): [M+H]⁺: 551

¹H NMR (300 MHz, DMSO-d₆) δ 10.29 (s, 1H), 9.51 (d, J=1.7 Hz, 1H), 8.66 (d, J=5.4 Hz, 2H), 8.45 (dd, J=15.0, 3.9 Hz, 2H), 8.12 (d, J=2.6 Hz, 1H), 7.51-7.37 (m, 1H), 7.32-7.20 (m, 1H), 4.11 (d, J=6.9 Hz, 2H), 2.88-2.80 (m, 3H), 1.89 (dt, J=13.7, 6.6 Hz, 1H), 0.87 (d, J=6.7 Hz, 6H).

### Example 192: Synthesis of N-[3-[1-(5-cyclopropyl-3H-imidazol-4-yl)imidazo[1,5-a]pyrazin-6-yl]-2,4-difluorophenyl]-5-fluoro-2-methoxypyridine-3-sulfonamide (Compound 177)

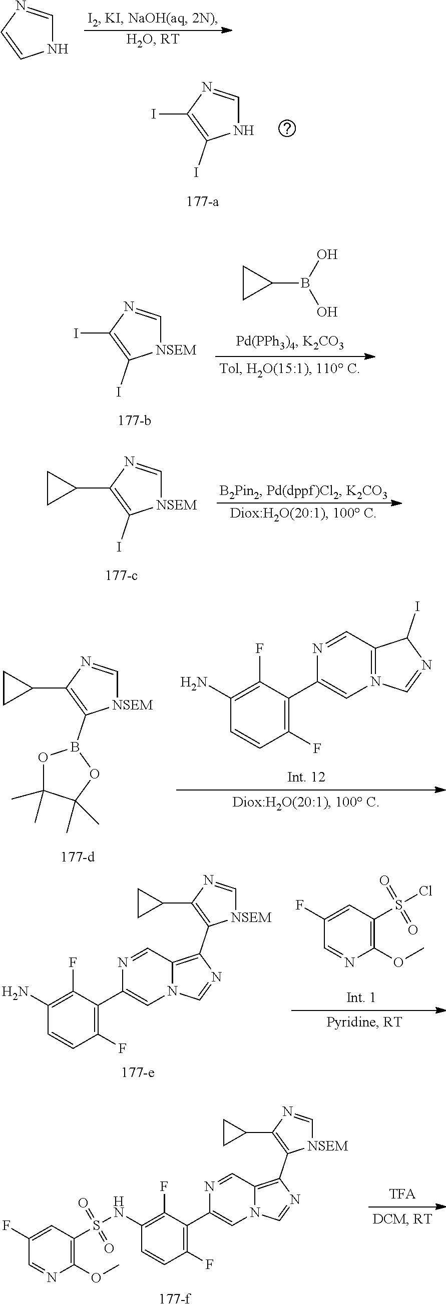

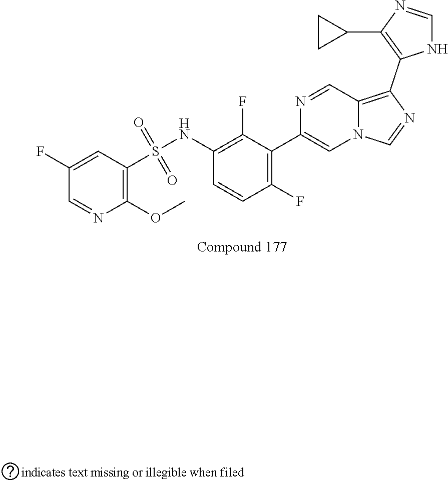 [SMILES: COc1ncc(F)cc1S(=O)(=O)Nc1ccc(F)c(-c2cn3cnc(-c4[nH]cnc4C4CC4)c3cn2)c1F]

### Synthesis of 177-a: 4,5-diiodo-1H-imidazole

A mixture of KI (29.3 g, 176 mmol, 4 equiv) and 1₂ (22.4 g, 88 mmol, 2 equiv) in water (60 mL) was added to a solution of imidazole (3 g, 44 mmol, 1 equiv) in 2 M aqueous NaOH (133 mL, 266 mmol, 6 equiv). The mixture was stirred for 3 h and then neutralized with 6 M HCl. The resulting solid was collected by filtration and recrystallized from ethanol (100 mL) to give 4,5-diiodo-1H-imidazole (14 g, 99% yield) as a grey solid.

LCMS (ES, m/z): [M+H]⁺: 321

### Synthesis of 177-b: 4,5-diiodo-1-[[2-(trimethylsilyl)ethoxy]methyl]imidazole

To 4,5-diiodo-1H-imidazole (6 g, 18.8 mmol, 1 equiv) in THE (60 mL) was added NaH (1.3 g, 20.1 mmol, 1.1 equiv) at 0° C. The mixture was stirred for 30 min, then SEM-C1 (4.07 g, 24.4 mmol, 1.3 equiv) was added dropwise. The reaction was stirred for 3 h at 0° C., then quenched by the addition of ice/water (150 mL). The resulting solution was extracted with ethyl acetate (3×100 mL), and the combined organics were washed with water (200 mL) and brine (200 mL), before being dried over anhydrous sodium sulfate and concentrated. The residue was applied to a silica gel column, eluting with ethyl acetate/petroleum ether (1:3), to give 4,5-diiodo-1-[[2-(trimethylsilyl)ethoxy]methyl]imidazole (6 g, 71% yield) as a light yellow oil.

LCMS (ES, m/z): [M+H]⁺: 451

### Synthesis of 177-c: 4-cyclopropyl-5-iodo-1-[[2-(trimethylsilyl)ethoxy]methyl]imidazole

4,5-Diiodo-1-[[2-(trimethylsilyl)ethoxy]methyl]imidazole (2.5 g, 5.6 mmol, 1 equiv), cyclopropylboronic acid (1.43 g, 17 mmol, 3 equiv), Pd(PPh₃)₄ (1.28 g, 1.1 mmol, 0.2 equiv) and K₂CO₃ (1.92 g, 14 mmol, 2.5 equiv) in toluene (50 mL) and H₂O (3 mL) were stirred for 1 day at 120° C. The resulting mixture was concentrated under vacuum. The crude product was purified by Flash-Prep-HPLC with the following conditions: Column, WelFlash™ C18-I, Spherical C18 20-40 m, 120 g; mobile phase: 20-85% MeCN/0.1% aqueous formic acid; to give 4-cyclopropyl-5-iodo-1-[[2-(trimethylsilyl)ethoxy]methyl]imidazole (0.26 g, 13% yield) as a light brown semi-solid.

LCMS (ES, m/z): [M+H]⁺: 365

### Synthesis of 177-d: 4-cyclopropyl-5-(4,4,5,5-tetramethyl-1,3,2-dioxaborolan-2-yl)-1-((2-(trimethylsilyl)ethoxy)methyl)-1H-imidazole

4-Cyclopropyl-5-iodo-1-[[2-(trimethylsilyl)ethoxy]methyl]imidazole (260 mg, 0.71 mmol, 1 equiv), bis(pinacolato)diboron (363 mg, 1.4 mmol, 2 equiv), Pd₂(dba)₃ (130 mg, 0.14 mmol, 0.2 equiv), PCy₃ (60 mg, 0.21 mmol, 0.3 equiv) and K₂CO₃ (197 mg, 1.4 mmol, 2 equiv) in dioxane (10 mL) were stirred for 5 h at 100° C. The reaction mixture was used in the next step directly.

LCMS (ES, m/z): [M+H]⁺: 365

### Synthesis of 177-e: 3-[i-(5-cyclopropyl-3-[[2-(trimethylsilyl)ethoxy]methyl]imidazol-4-yl)imidazo[1,5-a]pyrazin-6-yl]-2,4-difluoroaniline

To the crude solution of 4-cyclopropyl-5-(4,4,5,5-tetramethyl-1,3,2-dioxaborolan-2-yl)-1-[[2-(trimethylsilyl)ethoxy]methyl]imidazole from the previous step was added 2,4-difluoro-3-[1-iodoimidazo[1,5-a]pyrazin-6-yl]aniline (153 mg, 0.41 mmol, 1.5 equiv), K₂CO₃ (76 mg, 0.55 mmol, 2 equiv), H₂O (0.5 mL) and Pd(dppf)Cl₂ CH₂Cl₂ (45 mg, 0.055 mmol, 0.2 equiv). The resulting solution was stirred overnight at 100° C., then concentrated. The crude product was purified by Flash-Prep-HPLC with the following conditions: Column, WelFlash™ C18-I, Spherical C18 20-40 m, 120 g; mobile phase: 10-85% MeCN/0.1% aqueous ammonia; to give 3-[1-(5-cyclopropyl-3-[[2-(trimethylsilyl)ethoxy]methyl]imidazol-4-yl)imidazo[1,5-a]pyrazin-6-yl]-2,4-difluoroaniline (100 mg 76% yield) as a light brown solid.

LCMS (ES, m/z): [M+H]⁺: 483

### Synthesis of 177-f: N-[3-[1-(5-cyclopropyl-3-[[2-(trimethylsilyl)ethoxy]methyl]imidazol-4-yl)imidazo[1,5-a]pyrazin-6-yl]-2,4-difluorophenyl]-5-fluoro-2-methoxypyridine-3-sulfonamide

Into a 40 mL vial was placed 3-[1-(5-cyclopropyl-3-[[2-(trimethylsilyl)ethoxy]methyl]imidazol-4-yl)imidazo[1,5-a]pyrazin-6-yl]-2,4-difluoroaniline (130 mg, 0.27 mmol, 1 equiv), pyridine (5 mL) and 5-fluoro-2-methoxypyridine-3-sulfonyl chloride (122 mg, 0.54 mmol, 2 equiv). The resulting solution was stirred overnight, then concentrated. The crude product was purified by Flash-Prep-HPLC with the following conditions: Column, WelFlash™ C18-I, Spherical C18 20-40 m, 120 g; mobile phase: 10-80% MeCN/0.1% aqueous TFA; to give N-[3-[1-(5-cyclopropyl-3-[[2-(trimethylsilyl)ethoxy]methyl]imidazol-4-yl)imidazo[1,5-a]pyrazin-6-yl]-2,4-difluorophenyl]-5-fluoro-2-methoxypyridine-3-sulfonamide (130 mg 72% yield) as a light brown semi-solid.

LCMS (ES, m/z): [M+H]⁺: 672

### Synthesis of Compound 177: N-[3-[1-(5-cyclopropyl-3H-imidazol-4-yl)imidazo[1,5-a]pyrazin-6-yl]-2,4-difluorophenyl]-5-fluoro-2-methoxypyridine-3-sulfonamide

Into a 40 mL vial was placed N-[3-[1-(5-cyclopropyl-3-[[2-(trimethylsilyl)ethoxy]methyl]imidazol-4-yl)imidazo[1,5-a]pyrazin-6-yl]-2,4-difluorophenyl]-5-fluoro-2-methoxypyridine-3-sulfonamide (100 mg, 0.15 mmol, 1 equiv), DCM (3 mL) and TFA (1 mL). The resulting solution was stirred overnight then concentrated. The crude product was purified by Prep-HPLC with the following conditions: Column, welch Vltimate XB-C18, 50×250 mm, 10 μm; Mobile Phase: 10-55% MeCN/0.1% aqueous ammonia; to give N-[3-[1-(5-cyclopropyl-3H-imidazol-4-yl)imidazo[1,5-a]pyrazin-6-yl]-2,4-difluorophenyl]-5-fluoro-2-methoxypyridine-3-sulfonamide (55 mg, 68% yield) as a yellow solid.

LCMS (ES, m/z): [M+H]⁺: 542

¹H NMR (300 MHz, DMSO-d₆) δ 11.73 (s, 1H), 10.42 (s, 1H), 9.58 (s, 1H), 8.55 (s, 1H), 8.49-8.40 (m, 2H), 8.03 (dd, J=7.3, 3.0 Hz, 1H), 7.61 (s, 1H), 7.38 (td, J=8.9, 5.9 Hz, 1H), 7.21 (dd, J=9.7, 8.2 Hz, 1H), 3.92 (s, 3H), 3.04 (s, 1H), 0.99 (d, J=8.2 Hz, 2H), 0.88-77 (m, 2H).

### Example 193: Synthesis of N-[3-[1-(4-benzyl-1H-imidazol-2-yl)imidazo[1,5-a]pyrazin-6-yl]-2,4-difluorophenyl]-5-fluoro-2-methoxypyridine-3-sulfonamide (Compound 178)

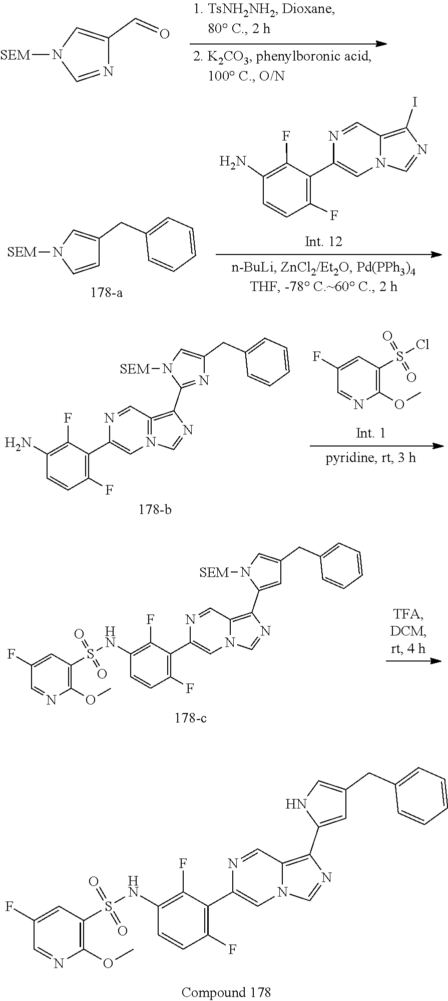

### Synthesis of 178-a: 4-benzyl-1-[[2-(trimethylsilyl)ethoxy]methyl]imidazole

A solution of 1-[[2-(trimethylsilyl)ethoxy]methyl]imidazole-4-carbaldehyde (2.7 g, 12 mmol, 1 equiv), and tosylhydrazine (2.2 g, 12 mmol, 1 equiv) in dioxane (100 ml) was stirred at 80° C. for 2 h. Potassium carbonate (4.95 g, 36 mmol, 3 eq) and phenylboronic (2.92 g, 24 mmol, 2 eq) were added to the reaction mixture, and this was heated at reflux for 12 h. Concentration gave a residue, which was applied to a silica gel column, eluting with PE:THF (2:1) resulting in 4-benzyl-1-[[2-(trimethylsilyl)ethoxy]methyl]imidazole (1.9 g, 48% yield) as a colorless oil.

LCMS (ES, m/z): [M+H]⁺: 289

### Synthesis of 178-b: 3-[I-(4-benzyl-1-[[2-(trimethylsilyl)ethoxy]methyl]imidazol-2-yl)imidazo[1,5-a]pyrazin-6-yl]-2,4-difluoroaniline

n-BuLi in hexanes (2.6 mL, 6.6 mmol, 3 equiv, 2.5 M) was added dropwise to a solution of 4-benzyl-1-[[2-(trimethylsilyl)ethoxy]methyl]imidazole (1.9 g, 6.6 mmol, 3 equiv) in THE (20 mL) at −78° C. The mixture was stirred at −78° C. for 30 minutes and then a solution of ZnCl₂ (6.6 mL, 6.6 mmol, 3 equiv, 1 M in Et₂O) was added dropwise at −78° C. The reaction mixture was allowed to warm to room temperature over 30 minutes, and 2,4-difluoro-3-[1-iodoimidazo[1,5-a]pyrazin-6-yl]aniline (800 mg, 2.2 mmol, 1 equiv) and Pd(PPh₃)₄ (497 mg, 0.44 mmol, 0.2 equiv) were added. The reaction was heated at 60° C. for 1 h, then cooled and quenched by the addition of methanol (5 mL). Concentration gave a residue, which was applied to a silica gel column, eluting with PE:EA (40:60) to give 3-[1-(4-benzyl-1-[[2-(trimethylsilyl)ethoxy]methyl]imidazol-2-yl)imidazo[1,5-a]pyrazin-6-yl]-2,4-difluoroaniline (630 mg, 44% yield) as a yellow oil.

LCMS (ES, m/z): [M+H]⁺: 533

### Synthesis of 178-c: N-[3-[1-(4-benzyl-1-[[2-(trimethylsilyl)ethoxy]methyl]imidazol-2-yl)imidazo[1,5-a]pyrazin-6-yl]-2,4-difluorophenyl]-5-fluoro-2-methoxypyridine-3-sulfonamide

Into an 8 mL round-bottom flask was placed 3-[1-(4-benzyl-1-[[2-(trimethylsilyl)ethoxy]methyl]imidazol-2-yl)imidazo[1,5-a]pyrazin-6-yl]-2,4-difluoroaniline (200 mg, 0.38 mmol, 1 equiv), pyridine (2 mL) and 5-fluoro-2-methoxypyridine-3-sulfonyl chloride (169 mg, 0.75 mmol, 2 equiv). The solution was stirred for 3 h then concentrated. The residue was applied to a silica gel column, eluting with ethyl acetate/petroleum ether (60%:40%) to give N-[3-[1-(4-benzyl-1-[[2-(trimethylsilyl)ethoxy]methyl]imidazol-2-yl)imidazo[1,5-a]pyrazin-6-yl]-2,4-difluorophenyl]-5-fluoro-2-methoxypyridine-3-sulfonamide (130 mg, 38% yield) as a yellow oil.

LCMS (ES, m/z): [M+H]⁺: 722

### Synthesis of Compound 178: N-[3-[1-(4-benzyl-1H-imidazol-2-yl)imidazo[1,5-a]pyrazin-6-yl]-2,4-difluorophenyl]-5-fluoro-2-methoxypyridine-3-sulfonamide

Into an 8 mL round-bottom flask was placed N-[3-[1-(4-benzyl-1-[[2-(trimethylsilyl)ethoxy]methyl]imidazol-2-yl)imidazo[1,5-a]pyrazin-6-yl]-2,4-difluorophenyl]-5-fluoro-2-methoxypyridine-3-sulfonamide (130 mg, 0.18 mmol, 1 equiv), DCM (3 mL) and TFA (1 mL). The resulting solution was stirred for 4 h then concentrated. The crude product was purified by Flash-Prep-HPLC with the following conditions: XBridge shield RP18 OBD, 19*150 mm 5 μm 10 nm; Mobile Phase: 15-38% MeCN/0.05% aqueous ammonia; to give N-[3-[1-(4-benzyl-1H-imidazol-2-yl)imidazo[1,5-a]pyrazin-6-yl]-2,4-difluorophenyl]-5-fluoro-2-methoxypyridine-3-sulfonamide (25 mg, 23% yield) as a yellow solid.

LCMS (ES, m/z): [M+H]⁺: 592

¹H NMR (300 MHz, DMSO-d₆) δ 12.58 (d, J=61.2 Hz, 1H), 10.43 (s, 1H), 9.52 (s, 1H), 8.64 (s, 1H), 8.53 (s, 1H), 8.45 (d, J=3.0 Hz, 1H), 8.02 (dd, J=7.3, 3.0 Hz, 1H), 7.44-7.27 (m, 5H), 7.26-7.15 (m, 2H), 6.86 (s, 1H), 3.85-4.05 (br s, 5H).

### Example 194: Synthesis of 6-[3-(5-chloro-2-methoxypyridine-3-sulfonamido)-2,6-difluorophenyl]-N-[2-(dimethylamino)ethyl]imidazo[1,5-a]pyrazine-1-carboxamide (Compound 179)

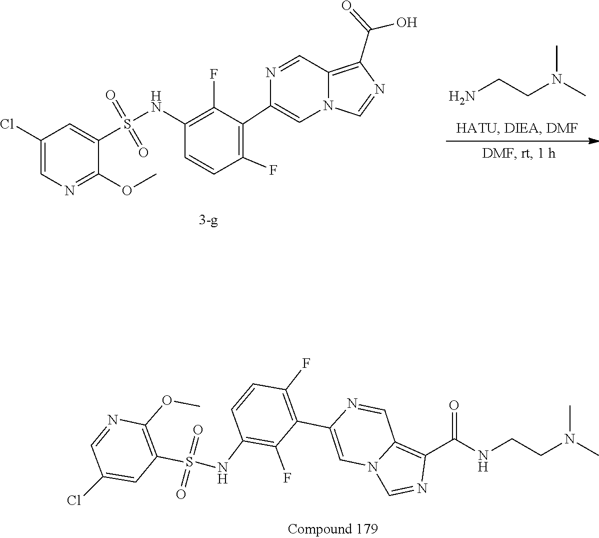

### Synthesis of Compound 179: 6-[3-(5-chloro-2-methoxypyridine-3-sulfonamido)-2,6-difluorophenyl]-N-[2-(dimethylamino)ethyl]imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added (2-aminoethyl)dimethylamine (53 mg, 0.6 mmol, 1.5 equiv) and HATU (230 mg, 0.6 mmol, 1.5 equiv). The resulting mixture was stirred for 1 h then diluted with H₂O (50 mL) and extracted with EA (3×50 mL). The combined organics were washed with brine (3×30 mL), dried over anhydrous Na₂SO₄ and concentrated. The crude product was purified by Prep-HPLC using the following conditions: Column: shim-pack C18, 20×150 mm, 5 μm; Mobile Phase: 10-32% MeCN/0.5% aqueous ammonia; to afford 6-[3-(5-chloro-2-methoxypyridine-3-sulfonamido)-2,6-difluorophenyl]-N-[2-(dimethylamino)ethyl]imidazo[1,5-a]pyrazine-1-carboxamide (60 mg, 26% yield) as a white solid.

LCMS (ES, m/z): [M+H]⁺: 566

¹H NMR (300 MHz, DMSO-d₆) δ 9.52 (d, J=1.6 Hz, 1H), 8.67 (d, J=2.4 Hz, 2H), 8.46-8.32 (m, 2H), 8.06 (d, J=2.7 Hz, 1H), 7.36 (td, J=9.0, 5.9 Hz, 1H), 7.22-7.04 (m, 1H), 3.88 (s, 3H), 3.50 (q, J=6.2 Hz, 2H), 2.70 (t, J=6.5 Hz, 2H), 2.40 (s, 6H).

### Example 195: Synthesis of 6-[3-(5-chloro-2-methoxypyridine-3-sulfonamido)-2,6-difluorophenyl]-N-[2-(2-oxopyrrolidin-1-yl)ethyl]imidazo[1,5-a]pyrazine-1-carboxamide (Compound 180)

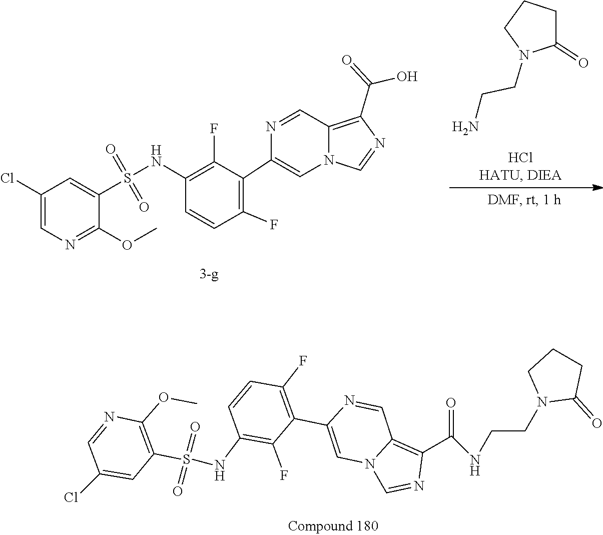 [SMILES: COc1ncc(Cl)cc1S(=O)(=O)Nc1ccc(F)c(-c2cn3cnc(C(=O)O)c3cn2)c1F ; NCCN1CCCC1=O ; COc1ncc(Cl)cc1S(=O)(=O)Nc1ccc(F)c(-c2cn3cnc(C(=O)NCCN4CCCC4=O)c3cn2)c1F]

### Synthesis of Compound 180: 6-[3-(5-chloro-2-methoxypyridine-3-sulfonamido)-2,6-difluorophenyl]-N-[2-(2-oxopyrrolidin-1-yl)ethyl]imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added 1-(2-aminoethyl)pyrrolidin-2-one hydrochloride (77 mg, 0.6 mmol, 1.5 equiv) and HATU (230 mg, 0.6 mmol, 1.5 equiv). The reaction was stirred for 1 h then diluted with H₂O (50 mL) and extracted with EA (3×50 mL). The combined organics were washed with brine (3×30 mL), dried over anhydrous Na₂SO₄ and concentrated. The crude product was purified by Prep-HPLC with following conditions: Column: XBridge Shield RP18 OBD, 5 μm, 19*150 mm; Mobile Phase: 15-35% MeCN/0.05% aqueous ammonia; to afford 6-[3-(5-chloro-2-methoxypyridine-3-sulfonamido)-2,6-difluorophenyl]-N-[2-(2-oxopyrrolidin-1-yl)ethyl]imidazo[1,5-a]pyrazine-1-carboxamide (70 mg, 29% yield) as a white solid.

LCMS (ES, m/z): [M+H]⁺: 606

¹H NMR (300 MHz, DMSO-d₆) δ 10.45 (s, 1H), 9.51 (d, J=1.6 Hz, 1H), 8.76-8.58 (m, 2H), 8.57-8.45 (m, 2H), 8.09 (d, J=2.6 Hz, 1H), 7.41 (td, J=8.9, 5.8 Hz, 1H), 7.23 (td, J=9.1, 1.6 Hz, 1H), 3.92 (s, 3H), 3.51-3.37 (m, 6H), 2.18 (dd, J=8.7, 7.4 Hz, 2H), 2.01-1.81 (m, 2H).

### Example 196: Synthesis of 6-[3-(5-chloro-2-methoxypyridine-3-sulfonamido)-2,6-difluorophenyl]-N-[4-[(dimethylamino)methyl]phenyl]imidazo[1,5-a]pyrazine-1-carboxamide (Compound 181)

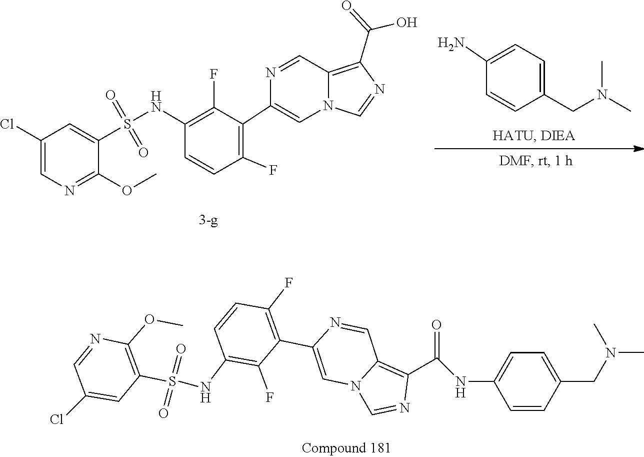 [SMILES: COc1ncc(Cl)cc1S(=O)(=O)Nc1ccc(F)c(-c2cn3cnc(C(=O)O)c3cn2)c1F ; CN(C)Cc1ccc(N)cc1 ; COc1ncc(Cl)cc1S(=O)(=O)Nc1ccc(F)c(-c2cn3cnc(C(=O)Nc4ccc(CN(C)C)cc4)c3cn2)c1F]

### Synthesis of Compound 180: 6-[3-(5-chloro-2-methoxypyridine-3-sulfonamido)-2,6-difluorophenyl]-N-[4-[(dimethylamino)methyl]phenyl]imidazo[1,5-a]pyrazine-1-carboxamide

To a stirred solution of 6-[3-(5-chloro-2-methoxypyridine-3-sulfonamido)-2,6-difluorophenyl]imidazo[1,5-a]pyrazine-1-carboxylic acid (200 mg, 0.4 mmol, 1 equiv) and DIEA (156 mg, 1.2 mmol, 3 equiv) in DMF (5 mL) was added 4-[(dimethylamino)methyl]aniline (90 mg, 0.6 mmol, 1.5 equiv) and HATU (230 mg, 0.6 mmol, 1.5 equiv) at room temperature. The resulting mixture was stirred for 1 h, then diluted with H₂O (50 mL) and extracted with EA (3×50 mL). The combined organic layers were washed with brine (3×30 mL), dried over anhydrous Na₂SO₄ and concentrated. The crude product was purified by Prep-HPLC with following conditions: Column: XBridge Shield RP18 OBD, 5 μm, 19*150 mm; Mobile Phase: 13-37% MeCN/0.05% aqueous ammonia; to afford 6-[3-(5-chloro-2-methoxypyridine-3-sulfonamido)-2,6-difluorophenyl]-N-[4-[(dimethylamino)methyl]phenyl]imidazo[1,5-a]pyrazine-1-carboxamide (32 mg, 13% yield) as a white solid.

LCMS (ES, m/z): [M+H]⁺: 628

¹H NMR (300 MHz, DMSO-d₆) δ 10.31 (s, 1H), 9.60 (d, J=1.6 Hz, 1H), 8.81-8.58 (m, 2H), 8.25 (dd, J=2.6, 1.1 Hz, 1H), 8.01 (d, J=2.6 Hz, 1H), 7.93-7.72 (m, 2H), 7.35-7.18 (m, 3H), 6.93 (t, J=9.2 Hz, 1H), 3.82 (s, 3H), 3.41 (s, 2H), 2.51 (p, J=1.9 Hz, 6H).

### Example 197: Synthesis of 6-[3-[5-chloro-2-(2,2,2-trifluoroethoxy)pyridine-3-sulfonamido]-2,6-difluorophenyl]-N-methylimidazo[1,5-a]pyrazine-1-carboxamide (Compound 182)

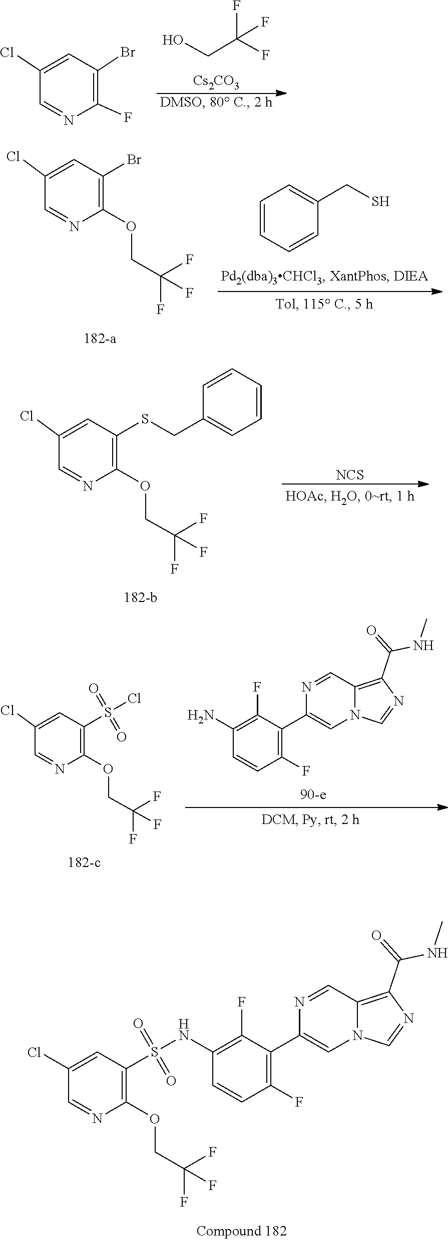

### Synthesis of 182-a: 3-bromo-5-chloro-2-(2,2,2-trifluoroethoxy)pyridine

To a stirred solution of 3-bromo-5-chloro-2-fluoropyridine (1 g, 4.8 mmol, 1 equiv) and trifluoroethanol (1.43 g, 14 mmol, 3 equiv) in DMSO (10 mL) was added cesium carbonate (4.66 g, 14 mmol, 3 equiv) in portions at room temperature. The reaction was stirred for 2 h at 80° C., then cooled and quenched with H₂O (30 mL). The mixture was extracted with EtOAc (3×10 mL), and the combined organics were washed with brine (20 mL), dried over anhydrous Na₂SO₄ and concentrated. The residue was purified by silica gel column chromatography, eluting with PE/EtOAc (50:1) to afford 3-bromo-5-chloro-2-(2,2,2-trifluoroethoxy)pyridine (1.2 g, 87% yield) as a light yellow solid.

LCMS (ES, m/z): [M+H]⁺: 290, 292

### Synthesis of 182-b: 3-(benzylsulfanyl)-5-chloro-2-(2,2,2-trifluoroethoxy)pyridine

To a stirred solution of 3-bromo-5-chloro-2-(2,2,2-trifluoroethoxy)pyridine (600 mg, 2.1 mmol, 1 equiv), benzyl mercaptan (385 mg, 3.1 mmol, 1.5 equiv) and DIEA (534 mg, 4.1 mmol, 2 equiv) in toluene (10 mL) were added XantPhos (239 mg, 0.41 mmol, 0.2 equiv) and Pd₂(dba)₃·CHCl₃ (214 mg, 0.21 mmol, 0.1 equiv) in portions at room temperature under nitrogen atmosphere. The reaction was stirred for 5 h at 115° C., then cooled and quenched with H₂O (30 mL). The resulting mixture was extracted with EtOAc (3×10 mL), and the combined organics were washed with brine (20 mL), dried over anhydrous Na₂SO₄ and concentrated. The residue was purified by prep-HPLC with the following conditions: Column, WelFlash™ C18-I, Spherical C18 20-40 m, 120 g; mobile phase: 40-80% MeCN/0.1% aqueous formic acid to afford 3-(benzylsulfanyl)-5-chloro-2-(2,2,2-trifluoroethoxy)pyridine (500 mg, 73% yield) as a yellow solid.

LCMS (ES, m/z): [M+H]⁺: 334

### Synthesis of 182-c: 5-chloro-2-(2,2,2-trifluoroethoxy)pyridine-3-sulfonyl chloride

To a stirred solution of 3-(benzylsulfanyl)-5-chloro-2-(2,2,2-trifluoroethoxy)pyridine (500 mg, 1.5 mmol, 1 equiv) in HOAc (9 mL) and H₂O (3 mL) was added NCS (700 mg, 5.2 mmol, 3.5 equiv) in portions at 0° C. The resulting mixture was quenched with H₂O (20 mL) and extracted with EtOAc (3×10 mL). The combined organics were washed with saturated NaHCO₃ solution (40 mL) and brine (1×20 mL), dried over anhydrous Na₂SO₄ and concentrated to give crude 5-chloro-2-(2,2,2-trifluoroethoxy)pyridine-3-sulfonyl chloride (650 mg) as a white solid.

### Synthesis of Compound 182: 6-[3-[5-chloro-2-(2,2,2-trifluoroethoxy)pyridine-3-sulfonamido]-2,6-difluorophenyl]-N-methylimidazo[1,5-a]pyrazine-1-carboxamide

To a stirred solution of 6-(3-amino-2,6-difluorophenyl)-N-methylimidazo[1,5-a]pyrazine-1-carboxamide (176 mg, 0.58 mmol, 0.3 equiv) in pyridine (5 mL) was added 5-chloro-2-(2,2,2-trifluoroethoxy)pyridine-3-sulfonyl chloride (600 mg, 1.9 mmol, 1 equiv) in DCM (0.5 mL) dropwise. The reaction was stirred for 2 h, then quenched with H₂O (30 mL) and extracted with EtOAc (3×10 mL). The combined organics were washed with brine (20 mL), dried over anhydrous Na₂SO₄ and concentrated. The residue was purified by prep-HPLC with the following conditions: Column, welch Vltimate XB-C18, 50×250 mm, 10 μm; Mobile Phase: 30-70% MeCN/0.1% aqueous formic acid; to afford 6-[3-[5-chloro-2-(2,2,2-trifluoroethoxy)pyridine-3-sulfonamido]-2,6-difluorophenyl]-N-methylimidazo[1,5-a]pyrazine-1-carboxamide (110 mg, 33% yield) as a white solid.

LCMS (ES, m/z): [M+H]⁺: 577

¹H NMR (300 MHz, DMSO-d₆) δ 10.51 (s, 1H), 9.51 (d, J=1.7 Hz, 1H), 8.65 (d, J=7.4 Hz, 2H), 8.49 (d, J=2.6 Hz, 1H), 8.41 (q, J=4.8 Hz, 1H), 8.18 (d, J=2.6 Hz, 1H), 7.38 (td, J=9.1, 5.9 Hz, 1H), 7.13 (t, J=8.6 Hz, 1H), 5.08 (q, J=8.9 Hz, 2H), 2.84 (d, J=4.8 Hz, 3H).

### Example 198: Synthesis of 6-[3-[2-(benzyloxy)-5-chloropyridine-3-sulfonamido]-2,6-difluorophenyl]-N-methylimidazo[1,5-a]pyrazine-1-carboxamide (Compound 183)

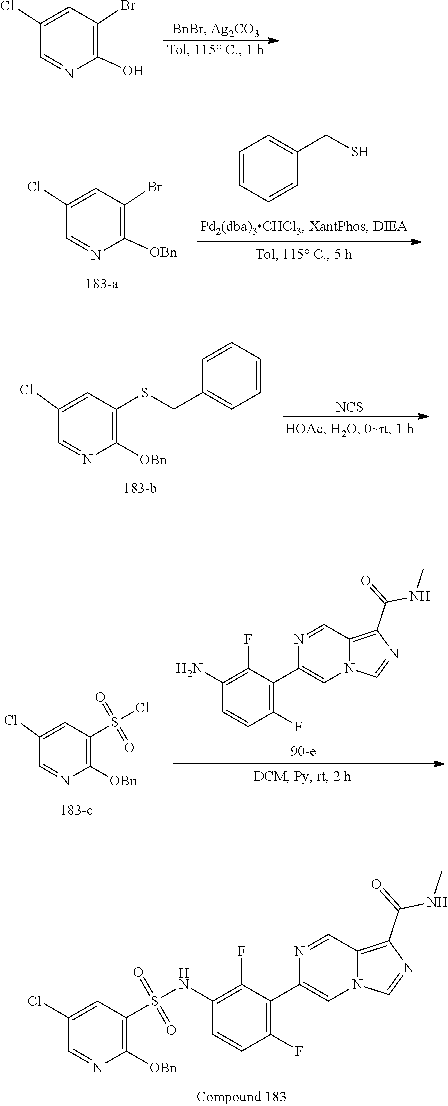

### Synthesis of 183-a: 2-(benzyloxy)-3-bromo-5-chloropyridine

A solution of 3-bromo-5-chloropyridin-2-ol (1 g, 4.8 mmol, 1 equiv), BnBr (0.9 g, 5.28 mmol, 1.1 equiv) and Ag₂CO₃ (1.46 g, 5.3 mmol, 1.1 equiv) in toluene (10 mL) was stirred for 1 h at 115° C. The reaction was cooled and quenched with H₂O (20 mL), then extracted with EtOAc (3×10 mL). The combined organics were washed with brine (20 mL), dried over anhydrous Na₂SO₄ and concentrated. The residue was purified by Flash-Prep-HPLC using the following conditions: Column, WelFlash™ C18-I, Spherical C18 20-40 m, 120 g; mobile phase: 40-90% MeCN/0.1% aqueous formic acid to afford 2-(benzyloxy)-3-bromo-5-chloropyridine (640 mg, 45% yield) as a light yellow solid.

LCMS (ES, m/z): [M+H]⁺: 298, 300

### Synthesis of 183-b: 2-(benzyloxy)-3-(benzylsulfanyl)-5-chloropyridine

To a stirred solution of 2-(benzyloxy)-3-bromo-5-chloropyridine (600 mg, 2.0 mmol, 1 equiv), XantPhos (233 mg, 0.4 mmol, 0.2 equiv) and benzyl mercaptan (374 mg, 3.0 mmol, 1.5 equiv) in toluene (10 mL) were added DIEA (520 mg, 4.0 mmol, 2 equiv) and Pd₂(dba)₃·CHCl₃ (208 mg, 0.2 mmol, 0.1 equiv) in portions at room temperature under nitrogen atmosphere. The resulting mixture was stirred for 5 h at 115° C., then cooled and quenched with H₂O (30 mL). The resulting biphasic mixture was extracted with EtOAc (3×10 mL), and the combined organics were washed with brine (30 mL), dried over anhydrous Na₂SO₄ and concentrated. The residue was purified by Flash-Prep-HPLC with the following conditions: Column, WelFlash™ C18-I, Spherical C18 20-40 m, 120 g; mobile phase: 50-95% MeCN/0.1% aqueous formic acid to afford 2-(benzyloxy)-3-(benzylsulfanyl)-5-chloropyridine (680 mg, 99% yield) as a light yellow solid.

LCMS (ES, m/z): [M+H]⁺: 342

### Synthesis of 183-c: 2-(benzyloxy)-5-chloropyridine-3-sulfonyl chloride

To a stirred solution of 2-(benzyloxy)-3-(benzylsulfanyl)-5-chloropyridine (690 mg, 2.02 mmol, 1 equiv) in HOAc (9 mL) and H₂O (3 mL) was added NCS (943 mg, 7.1 mmol, 3.5 equiv) in portions at 0° C. The resulting mixture was stirred for 0.5 h at room temperature, then quenched with H₂O (20 mL) and extracted with EtOAc (3×10 mL). The combined organics were washed with NaHCO₃ (40 mL) and brine (20 mL), dried over anhydrous Na₂SO₄ and concentrated to afford crude 2-(benzyloxy)-5-chloropyridine-3-sulfonyl chloride (1.1 g) as an off-white oil which was used in the next step directly without further purification.

### Synthesis of Compound 183: 6-[3-[2-(benzyloxy)-5-chloropyridine-3-sulfonamido]-2,6-difluorophenyl]-N-methylimidazo[1,5-a]pyrazine-1-carboxamide

To a stirred solution of 6-(3-amino-2,6-difluorophenyl)-N-methylimidazo[1,5-a]pyrazine-1-carboxamide (172 mg, 0.57 mmol, 0.3 equiv) in pyridine (6 mL) was added 2-(benzyloxy)-5-chloropyridine-3-sulfonyl chloride (600 mg, 1.9 mmol, 1 equiv) in DCM (0.6 mL) dropwise. The reaction was stirred for 2 h at room temperature. The resulting mixture was quenched with H₂O (20 mL) and extracted with EtOAc (3×10 mL). The combined organics were washed with brine (20 mL), dried over anhydrous Na₂SO₄ and concentrated. The residue was purified by prep-HPLC with the following conditions: Column, welch Vltimate XB-C18, 50×250 mm, 10 μm: Mobile Phase: 30-75% MeCN/0.1% aqueous formic acid; to afford 6-[3-[2-(benzyloxy)-5-chloropyridine-3-sulfonamido]-2,6-difluorophenyl]-N-methylimidazo[1,5-a]pyrazine-1-carboxamide (110 mg, 10% yield) as a white solid.

LCMS (ES, m/z): [M+H]⁺: 585 H NMR (300 MHz, DMSO-d₆) δ 10.46 (s, 1H), 9.52 (d, J=1.8 Hz, 1H), 8.68 (s, 1H), 8.57-8.47 (m, 2H), 8.43 (q, J=4.7 Hz, 1H), 8.16 (d, J=2.6 Hz, 1H), 7.44-7.32 (m, 3H), 7.37-7.19 (m, 3H), 7.10 (td, J=9.0, 1.6 Hz, 1H), 5.50 (s, 2H), 2.88-2.81 (m, 3H).

### Example 199: Synthesis of 6-[2,6-difluoro-3-(5-fluoro-2-methylpyridine-3-sulfonamido)phenyl]-5-fluoro-N-methylimidazo[1,5-a]pyrazine-1-carboxamide (Compound 184)

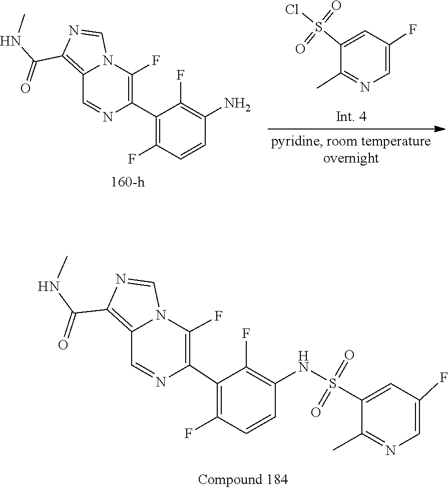 [SMILES: CNC(=O)c1ncn2c(F)c(-c3c(F)ccc(N)c3F)ncc12 ; Cc1ncc(F)cc1S(=O)(=O)Cl ; CNC(=O)c1ncn2c(F)c(-c3c(F)ccc(NS(=O)(=O)c4cc(F)cnc4C)c3F)ncc12]

### Synthesis of Compound 184: 6-[2,6-difluoro-3-(5-fluoro-2-methylpyridine-3-sulfonamido)phenyl]-5-fluoro-N-methylimidazo[1,5-a]pyrazine-1-carboxamide

To a stirred solution of 6-(3-amino-2,6-difluorophenyl)-5-fluoro-N-methylimidazo[1,5-a]pyrazine-1-carboxamide (60 mg, 0.2 mmol, 1 equiv) in pyridine (3 mL) was added 5-fluoro-2-methylpyridine-3-sulfonyl chloride (78 mg, 0.4 mmol, 2 equiv) in portions at room temperature. The resulting mixture was stirred overnight, then concentrated. The residue was purified by prep-HPLC with the following conditions: Column, welch Vltimate XB-C18, 50×250 mm, 10 m; Mobile Phase: 27-46% MeCN/0.1% aqueous formic acid; to give 6-[2,6-difluoro-3-(5-fluoro-2-methylpyridine-3-sulfonamido)phenyl]-5-fluoro-N-methylimidazo[1,5-a]pyrazine-1-carboxamide (51 mg, 55% yield) as an off-white solid.

LCMS (ES, m/z): [M+H]⁺: 495

¹H NMR (300 MHz, DMSO-d₆) δ 10.83 (s, 1H), 9.35 (d, J=2.8 Hz, 1H), 8.91 (s, 1H), 8.72 (d, J=2.8 Hz, 1H), 8.54 (q, J=4.7 Hz, 1H), 7.92 (dd, J=8.2, 2.8 Hz, 1H), 7.50 (td, J=8.9, 5.8 Hz, 1H), 7.30 (td, J=9.1, 1.5 Hz, 1H), 2.85 (d, J=4.7 Hz, 3H), 2.78 (d, J=1.2 Hz, 3H).

### Example 200: Synthesis of N-(2,4-difluoro-3-[1-[5-(4-methylpiperazin-1-yl)-3H-1,3-benzodiazol-2-yl]imidazo[1,5-a]pyridin-6-yl]phenyl)-5-fluoro-2-methoxypyridine-3-sulfonamide (Compound 185)

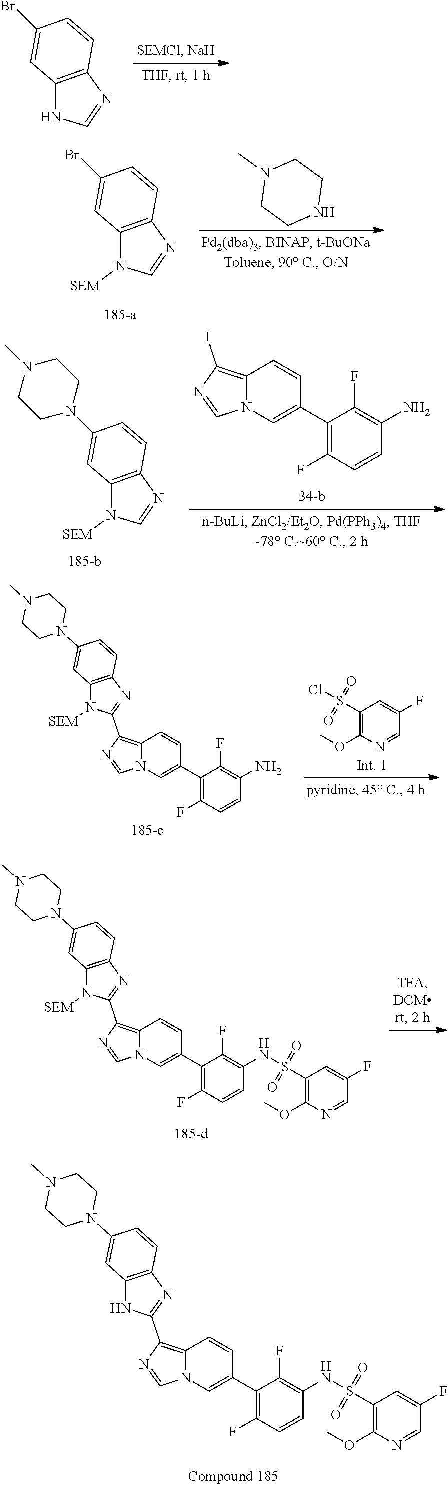

### Synthesis of 185-a: 6-bromo-1-[[2-(trimethylsilyl) ethoxy]methyl]-1,3-benzodiazole

To a solution of 5-bromo-3H-1,3-benzodiazole (5 g, 25 mmol, 1 equiv) in THF (130 mL) cooled in an ice bath was added NaH (1 g, 30 mmol, 1.2 equiv, 60% in oil) in portions. The mixture was stirred for 30 min at 0° C., then SEM-Cl (5 g, 30 mmol, 1.2 equiv) was added dropwise. The ice bath was removed and the solution was stirred for 1 h. Water (100 mL) was added and the mixture extracted with ethyl acetate (3×100 mL). The combined organics were washed with brine (100 mL), dried over anhydrous Na₂SO₄ and concentrated. The residue was purified by silica gel column chromatography, eluting with PE:EA (4:1) to afford 6-bromo-1-[[2-(trimethylsilyl) ethoxy]methyl]-1,3-benzodiazole (7 g, 86% yield) as a yellow oil.

LCMS (ES, m/z): [M+H]⁺: 327, 329

### Synthesis of 185-b: 6-(4-methylpiperazin-1-yl)-1-[[2-(trimethylsilyl)ethoxy]methyl]-1,3-benzodiazole

Into a 250 mL 3-necked round-bottom flask purged and maintained with an inert atmosphere of nitrogen was placed 6-bromo-1-[[2-(trimethylsilyl)ethoxy]methyl]-1,3-benzodiazole (4 g, 12 mmol, 1 equiv), toluene (100 mL), 1-methyl-piperazine (1.2 g, 12 mmol, 1 equiv), Pd₂(dba)₃·CHCl₃ (1.27 g, 1.2 mmol, 0.1 equiv), BINAP (0.76 g, 1.2 mmol, 0.1 equiv) and t-BuONa (3.5 g, 36 mmol, 3 equiv). The resulting solution was stirred for 12 h at 90° C. then concentrated. The residue was applied to a silica gel column, eluting with dichloromethane/methanol (1:10) to give 6-(4-methylpiperazin-1-yl)-1-[[2-(trimethylsilyl)ethoxy]methyl]-1,3-benzodiazole (2.9 g, 55% yield) as a colorless oil.

LCMS (ES, m/z): [M+H]⁺: 347

### Synthesis of 185-c: 2,4-difluoro-3-[1-[6-(4-methylpiperazin-1-yl)-1-[[2-(trimethylsilyl)ethoxy]methyl]-1,3-benzodiazol-2-yl]imidazo[1,5-a]pyridin-6-yl]aniline

Into a 20 mL 3-necked round-bottom flask, purged and maintained with an inert atmosphere of nitrogen, was placed 6-(4-methylpiperazin-1-yl)-1-[[2-(trimethylsilyl)ethoxy]methyl]-1,3-benzodiazole (1.4 g, 4.05 mmol, 3 equiv) in THE (20 mL). 2.5M n-BuLi in hexanes (1.6 mL, 4.1 mmol, 3 equiv) was added dropwise at −78° C., and the mixture was stirred at −78° C. for 30 minutes. ZnCl₂ solution (4.05 mL, 4.05 mmol, 3 equiv, 1 M in Et₂O) was added dropwise at −78° C. and the reaction allowed to warm to room temperature over 30 minutes. To the mixture was added Pd(PPh₃)₄ (311 mg, 0.27 mmol, 0.2 equiv) and 2,4-difluoro-3-[1-iodoimidazo[1,5-a]pyridin-6-yl]aniline (500 mg, 1.35 mmol, 1 equiv). The resulting solution was stirred for 2 h at 60° C., then cooled and quenched by the addition of MeOH (5 mL). The reaction was concentrated and the residue applied to a silica gel column, eluting with dichloromethane/methanol (10:1) to give 2,4-difluoro-3-[1-[6-(4-methylpiperazin-1-yl)-1-[[2-(trimethylsilyl)ethoxy]methyl]-1,3-benzodiazol-2-yl]imidazo[1,5-a]pyridin-6-yl]aniline (500 mg, 57% yield) as a yellow solid.

LCMS (ES, m/z): [M+H]⁺: 590

### Synthesis of 185-d: methyl N-(2,4-difluoro-3-[1-[6-(4-methylpiperazin-1-yl)-1-[[2-(trimethylsilyl)ethoxy]methyl]-1H-1,3-benzodiazol-2-yl]imidazo[1,5-a]pyridin-6-yl]phenyl)-5-fluoro-2-methoxypyridine-3-sulfonamide

Into an 8 mL round-bottom flask was placed 2,4-difluoro-3-[1-[6-(4-methylpiperazin-1-yl)-1-[[2-(trimethylsilyl)ethoxy]methyl]-1H-1,3-benzodiazol-2-yl]imidazo[1,5-a]pyridin-6-yl]aniline (150 mg, 0.25 mmol, 1 equiv), pyridine (2 mL) and 5-fluoro-2-methoxypyridine-3-sulfonyl chloride (115 mg, 0.5 mmol, 2 equiv). The resulting solution was stirred for 3 h then concentrated under vacuum. The residue was applied to a silica gel column, eluting with dichloromethane/methanol (10:1) to give crude N-(2,4-difluoro-3-[1-[6-(4-methylpiperazin-1-yl)-1-[[2-(trimethylsilyl)ethoxy]methyl]-1H-1,3-benzodiazol-2-yl]imidazo[1,5-a]pyridin-6-yl]phenyl)-5-fluoro-2-methoxypyridine-3-sulfonamide (137 mg) as a yellow oil.

LCMS (ES, m/z): [M+H]⁺: 779

### Synthesis of Compound 185: methyl N-(2,4-difluoro-3-[1-[5-(4-methylpiperazin-1-yl)-3H-1,3-benzodiazol-2-yl]imidazo[1,5-a]pyridin-6-yl]phenyl)-5-fluoro-2-methoxypyridine-3-sulfonamide

Into an 8 mL round-bottom flask was placed N-(2,4-difluoro-3-[1-[6-(4-methylpiperazin-1-yl)-1-[[2-(trimethylsilyl)ethoxy]methyl]-1,3-benzodiazol-2-yl]imidazo[1,5-a]pyridin-6-yl]phenyl)-5-fluoro-2-methoxypyridine-3-sulfonamide (137 mg, 0.18 mmol, 1 equiv), DCM (3 mL) and TFA (1 mL). The resulting solution was stirred for 2 h, then concentrated under vacuum. The crude product was purified by Flash-Prep-HPLC with the following conditions: XBridge shield RP18 OBD, 19*150 mm 5 μm 10 nm; Mobile Phase: 20-45% MeCN/0.05% aqueous ammonia; to give N-(2,4-difluoro-3-[1-[5-(4-methylpiperazin-1-yl)-3H-1,3-benzodiazol-2-yl]imidazo[1,5-a]pyridin-6-yl]phenyl)-5-fluoro-2-methoxypyridine-3-sulfonamide (14 mg, 12% yield) as a yellow solid.

LCMS (ES, m/z): [M+H]⁺: 649

¹H NMR (300 MHz, DMSO-d₆) δ 12.47 (s, 1H), 8.61 (d, J=3.5 Hz, 2H), 8.40 (q, J=3.3, 2.4 Hz, 2H), 8.00 (dd, J=7.5, 3.0 Hz, 1H), 7.34 (td, J=8.9, 5.9 Hz, 2H), 7.21-6.89 (m, 4H), 3.90 (s, 3H), 3.14 (t, J=5.0 Hz, 4H), 2.60 (s, 4H), 2.32 (s, 3H).

### Example 201: Synthesis of 6-[3-(5-chloro-2-methylpyridine-3-sulfonamido)-2,6-difluorophenyl]-5-fluoro-N-methylimidazo[1,5-a]pyrazine-1-carboxamide (Compound 186)

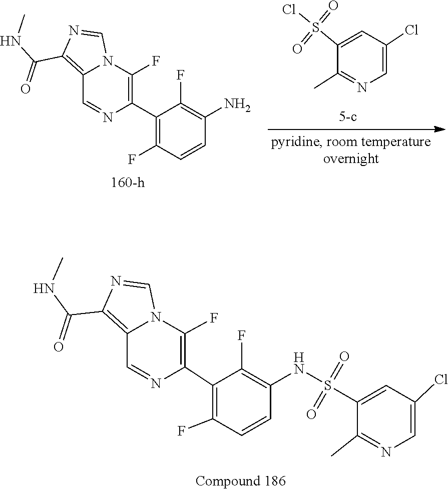 [SMILES: CNC(=O)c1ncn2c(F)c(-c3c(F)ccc(N)c3F)ncc12 ; Cc1ncc(Cl)cc1S(=O)(=O)Cl ; CNC(=O)c1ncn2c(F)c(-c3c(F)ccc(NS(=O)(=O)c4cc(Cl)cnc4C)c3F)ncc12]

### Synthesis of Compound 186: 6-[3-(5-chloro-2-methylpyridine-3-sulfonamido)-2,6-difluorophenyl]-5-fluoro-N-methylimidazo[1,5-a]pyrazine-1-carboxamide

To a stirred solution of 6-(3-amino-2,6-difluorophenyl)-5-fluoro-N-methylimidazo[1,5-a]pyrazine-1-carboxamide (60 mg, 0.2 mmol, 1 equiv) in pyridine (3 mL) was added 5-chloro-2-methylpyridine-3-sulfonyl chloride (84 mg, 0.4 mmol, 2 equiv) in portions at room temperature. The reaction was stirred overnight then concentrated. The residue was purified by prep-HPLC with the following conditions: Column, welch Vltimate XB-C18, 50×250 mm, 10 am; Mobile Phase: 31-46% M2CN/0.1% aqueous formic acid; to give 6-[3-(5-chloro-2-methylpyridine-3-sulfonamido)-2,6-difluorophenyl]-5-fluoro-N-methylimidazo[1,5-a]pyrazine-1-carboxamide (32 mg, 340 yield) as a white solid.

LCMS (ES, m/z): [M+H]⁺: 511

¹H NMR (300 MHz, DMSO-d₆) δ 10.83 (s, 1H), 9.35 (d, J=2.8 Hz, 1H), 8.91 (s, 1H), 8.74 (d, J=2.4 Hz, 1H), 8.54 (d, J=4.9 Hz, 1H), 8.06 (d, J=2.4 Hz, 1H), 7.49 (td, J=9.0, 5.8 Hz, 1H), 7.28 (t, J=9.1 Hz, 1H), 2.85 (d, J=4.7 Hz, 3H), 2.77 (s, 3H).

TABLE 1

| Cmpd | | | | |
|---|---|---|---|---|
| No. | | Structure | Int. | LC-MS |
| 1 | 6-[3-(5-chloro-2- methoxypyridine-3- sulfonamido)-2,6- difluorophenyl]-N- methylimidazo[1,5- a]pyridine-1- carboxamide | 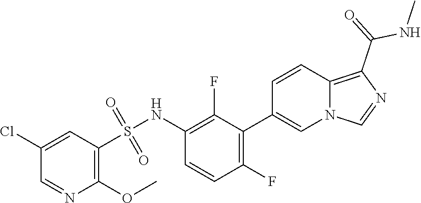 [SMILES: CNC(=O)c1ncn2cc(-c3c(F)ccc(NS(=O)(=O)c4cc(Cl)cnc4OC)c3F)ccc12] | 6, 5 | [M + H]⁺: 508 |
| 2 | 5-chloro-N-[2,4-difluoro- 3-[1-(1H-imidazol-2- yl)imidazo[1,5-a]pyridin- 6-yl]phenyl]-2- methoxypyridine-3- sulfonamide | 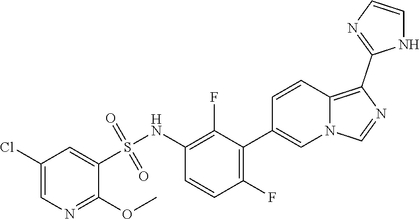 [SMILES: COc1ncc(Cl)cc1S(=O)(=O)Nc1ccc(F)c(-c2ccc3c(-c4ncc[nH]4)ncn3c2)c1F] | 5 | [M + H]⁺: 517 |
| 3 | 6-[3-(5-Chloro-2- methoxypyridine-3- sulfonamido)-2,6- difluorophenyl]-N- methylimidazo[1,5- a]pyrazine-1- carboxamide | 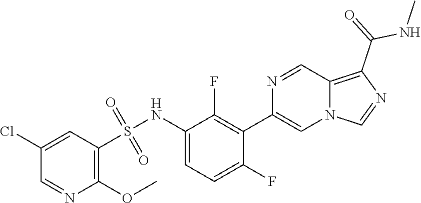 [SMILES: CNC(=O)c1ncn2cc(-c3c(F)ccc(NS(=O)(=O)c4cc(Cl)cnc4OC)c3F)ncc12] | 2 | [M + H]⁺: 509 |
| 4 | 2-[3-(5-Chloro-2- methoxypyridine-3- sulfonamido)-2,6- difluorophenyl]-N- methylimidazo[1,5- b]pyridazine-5- carboxamide | 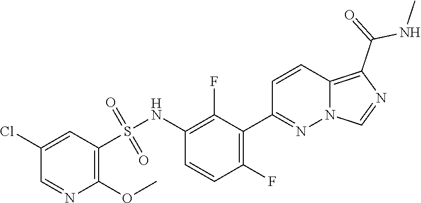 [SMILES: CNC(=O)c1ncn2nc(-c3c(F)ccc(NS(=O)(=O)c4cc(Cl)cnc4OC)c3F)ccc12] | 2 | [M + H]⁺: 509 |
| 5 | 2-[3-(5-Chloro-2- methylpyridine-3- sulfonamido)-2,6- difluorophenyl]-N- methylimidazo[1,5- b]pyridazine-5- carboxamide | 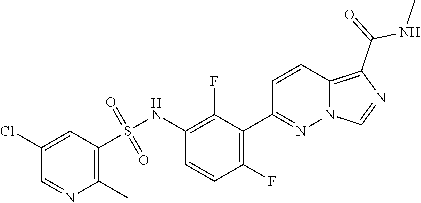 [SMILES: CNC(=O)c1ncn2nc(-c3c(F)ccc(NS(=O)(=O)c4cc(Cl)cnc4C)c3F)ccc12] | | [M + H]⁺: 493 |
| 6 | 6-[2,6-difluoro-3-(5- fluoro-2-methylpyridine- 3-sulfonamido)phenyl]- N-methylimidazo[1,5- a]pyridine-1- carboxamide | 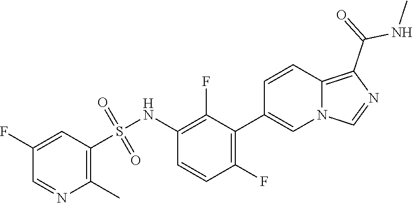 [SMILES: CNC(=O)c1ncn2cc(-c3c(F)ccc(NS(=O)(=O)c4cc(F)cnc4C)c3F)ccc12] | 4 | [M + H]⁺: 476 |
| 7 | 6-[2,6-difluoro-3-(5- fluoro-2- methoxypyridine-3- sulfonamido)phenyl]-N- methylimidazo[1,5- a]pyridine-1- carboxamide | 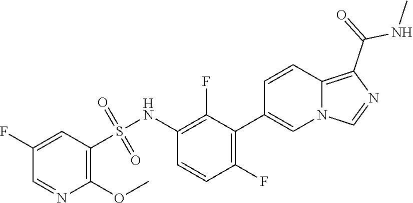 [SMILES: CNC(=O)c1ncn2cc(-c3c(F)ccc(NS(=O)(=O)c4cc(F)cnc4OC)c3F)ccc12] | 1, 16 | [M + H]⁺: 491 |
| 8 | 6-[3-(5-cyano-2- methoxypyridine-3- sulfonamido)-2,6- difluorophenyl]-N- methylimidazo[1,5- a]pyridine-1- carboxamide | 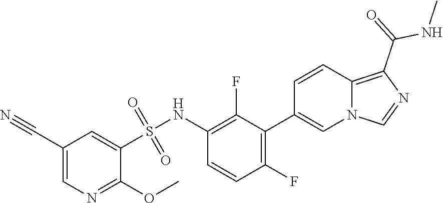 [SMILES: CNC(=O)c1ncn2cc(-c3c(F)ccc(NS(=O)(=O)c4cc(C#N)cnc4OC)c3F)ccc12] | 3, 16 | [M + H]⁺: 498 |
| 9 | 6-[3-(5-cyano-2- methylpyridine-3- sulfonamido)-2,6- difluorophenyl]-N- methylimidazo[1,5- a]pyridine-1- carboxamide | 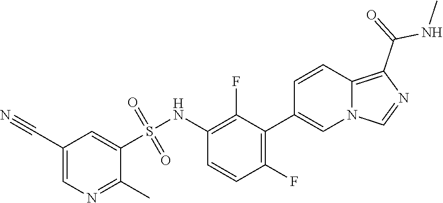 [SMILES: CNC(=O)c1ncn2cc(-c3c(F)ccc(NS(=O)(=O)c4cc(C#N)cnc4C)c3F)ccc12] | | [M + H]⁺: 483 |
| 10 | 6-[2,6-difluoro-3-(6- fluoro-1-hydroxy-2,3- dihydro-1H-indene-4- sulfonamido)phenyl]-N- methylimidazo[1,5- a]pyridine-1- carboxamide | 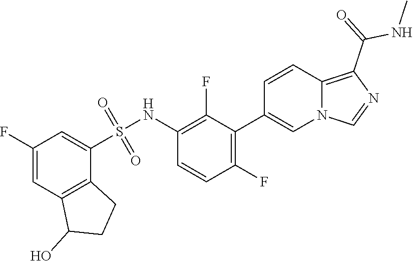 [SMILES: CNC(=O)c1ncn2cc(-c3c(F)ccc(NS(=O)(=O)c4cc(F)cc5c4CCC5O)c3F)ccc12] | | [M + H]⁺: 517 |
| 11 | 6-[3-(5-chloro-2- methoxypyridine-3- sulfonamido)-2,6- difluorophenyl]-N,5- dimethylimidazo[1,5- a]pyridine-1- carboxamide | 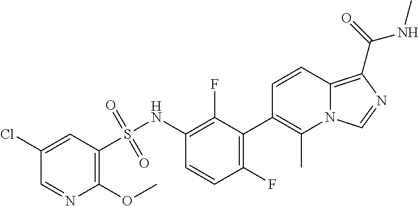 [SMILES: CNC(=O)c1ncn2c(C)c(-c3c(F)ccc(NS(=O)(=O)c4cc(Cl)cnc4OC)c3F)ccc12] | 5 | [M + H]⁺: 522 |
| 12 | 2-[2,6-difluoro-3-(5- fluoro-2-methylpyridine- 3-sulfonamido)phenyl]- N-methylimidazo[1,5- b]pyridazine-5- carboxamide | 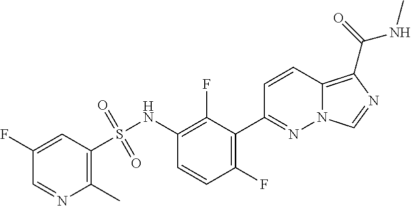 [SMILES: CNC(=O)c1ncn2nc(-c3c(F)ccc(NS(=O)(=O)c4cc(F)cnc4C)c3F)ccc12] | 4 | [M + H]⁺: 477 |
| 13 | 6-[2,6-difluoro-3-[2- methoxy-5- (trifluoromethyl) pyridine-3-sulfonamido] phenyl]-N- methylimidazo[1,5-a] pyridine-1- carboxamide | 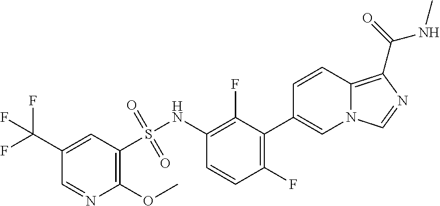 [SMILES: CNC(=O)c1ncn2cc(-c3c(F)ccc(NS(=O)(=O)c4cc(C(F)(F)F)cnc4OC)c3F)ccc12] | 16 | [M + H]⁺: 542 |
| 14 | 6-[3-(5-chloro-2- methoxypyridine-3- sulfonamido)-2,6- difluorophenyl]-5- fluoro-N- methylimidazo[1,5- a]pyridine-1- carboxamide | 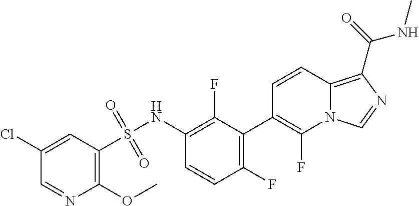 [SMILES: CNC(=O)c1ncn2c(F)c(-c3c(F)ccc(NS(=O)(=O)c4cc(Cl)cnc4OC)c3F)ccc12] | 2 | [M + H]⁺: 526 |
| 15 | 6-[3-(5-chloro-2- methoxypyridine-3- sulfonamido)-2,6- difluorophenyl]-5- methoxy-N- methylimidazo[1,5- a]pyridine-1- carboxamide | 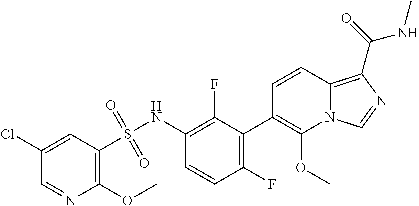 [SMILES: CNC(=O)c1ncn2c(OC)c(-c3c(F)ccc(NS(=O)(=O)c4cc(Cl)cnc4OC)c3F)ccc12] | 5 | [M + H]⁺: 538 |
| 16 | 3-[3-(5-Chloro-2- methoxypyridine-3- sulfonamido)-2,6- difluorophenyl]-N- methylimidazo[1,5- a]pyrimidine-8- carboxamide | 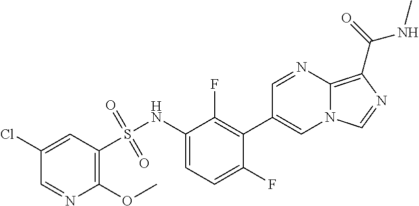 [SMILES: CNC(=O)c1ncn2cc(-c3c(F)ccc(NS(=O)(=O)c4cc(Cl)cnc4OC)c3F)cnc12] | 5 | [M + H]⁺: 509 |
| 17 | 3-[3-(5-chloro-2- methoxypyridine-3- sulfonamido)-2,6- difluorophenyl]-2- methoxy-N- methylimidazo[1,5- a]pyrimidine-8- carboxamide | 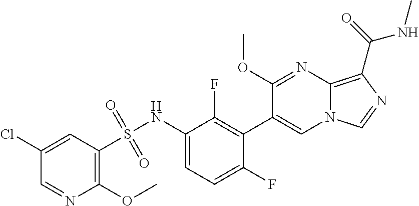 [SMILES: CNC(=O)c1ncn2cc(-c3c(F)ccc(NS(=O)(=O)c4cc(Cl)cnc4OC)c3F)c(OC)nc12] | 2 | [M + H]⁺: 539 |
| 18 | N-[2,4-difluoro-3-[1- (1H-imidazol-2- yl)imidazo[1,5-a] pyridin-6-yl]phenyl]-5- fluoro-2-methoxy- pyridine-3- sulfonamide | 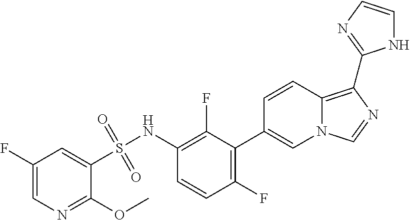 [SMILES: COc1ncc(F)cc1S(=O)(=O)Nc1ccc(F)c(-c2ccc3c(-c4ncc[nH]4)ncn3c2)c1F] | 1, 6 | [M + H]⁺: 501 |
| 19 | N-[2,4-difluoro-3-[1- (1H-imidazol-2- yl)imidazo[1,5-a] pyridin-6-yl]phenyl]-5- fluoro-2- methylpyridine-3- sulfonamide | 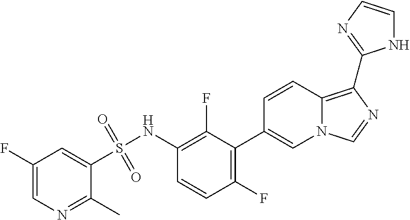 [SMILES: Cc1ncc(F)cc1S(=O)(=O)Nc1ccc(F)c(-c2ccc3c(-c4ncc[nH]4)ncn3c2)c1F] | 4 | [M + H]⁺: 485 |
| 20 | 5-Cyano-N-[2,4-difluoro- 3-[1-(1H-imidazol-2- yl)imidazo[1,5-a]pyridin- 6-yl]phenyl]-2- methoxypyridine-3- sulfonamide | 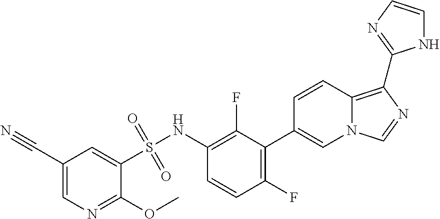 [SMILES: COc1ncc(C#N)cc1S(=O)(=O)Nc1ccc(F)c(-c2ccc3c(-c4ncc[nH]4)ncn3c2)c1F] | 3 | [M + H]⁺: 508 |
| 21 | 7-[3-(5-chloro-2- methoxypyridine-3- sulfonamido)-2,6- difluorophenyl]-8-fluoro- N-methylimidazo[1,5- a]pyridine-3- carboxamide | 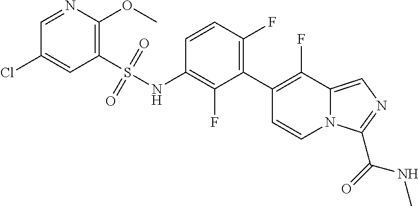 [SMILES: CNC(=O)c1ncc2c(F)c(-c3c(F)ccc(NS(=O)(=O)c4cc(Cl)cnc4OC)c3F)ccn12] | 2 | [M + H]⁺: 526 |
| 22 | 3-[3-(5-Chloro-2- methoxypyridine-3- sulfonamido)-2,6- difluorophenyl]-N,1- dimethyl-2- oxoimidazol[1,5-a] pyrimidine-8- carboxamide | 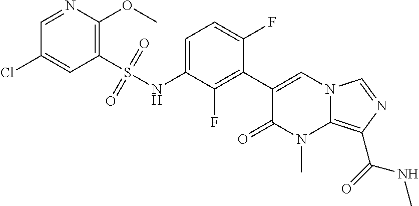 [SMILES: CNC(=O)c1ncn2cc(-c3c(F)ccc(NS(=O)(=O)c4cc(Cl)cnc4OC)c3F)c(=O)n(C)c12] | 2 | [M + H]⁺: 539 |
| 23 | 6-[3-[3-cyano-5- (trifluoromethyl)benzene sulfonamido]-2,6- difluorophenyl]-N- methylimidazo[1,5- a]pyridine-1- carboxamide | 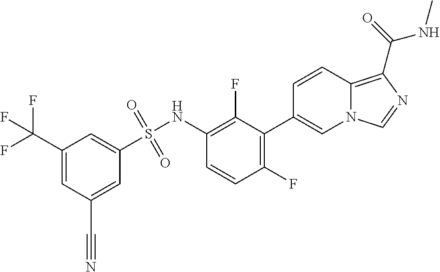 [SMILES: CNC(=O)c1ncn2cc(-c3c(F)ccc(NS(=O)(=O)c4cc(C#N)cc(C(F)(F)F)c4)c3F)ccc12] | 10 | [M + H]⁺: 536 |
| 24 | 6-[3-(5-chloro-2- methoxypyridine-3- sulfonamido)-2,6- difluorophenyl]-N- methyl-5- (methylamino)imidazo [1,5-a]pyridine-1- carboxamide | 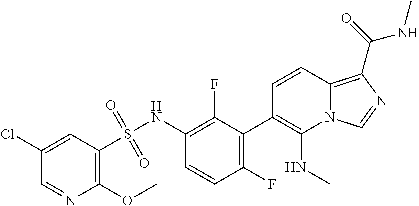 [SMILES: CNC(=O)c1ncn2c(NC)c(-c3c(F)ccc(NS(=O)(=O)c4cc(Cl)cnc4OC)c3F)ccc12] | | [M + H]⁺: 537 |
| 25 | 5-Chloro-N-[2,4- difluoro-3-[1-(1H- imidazol-2- yl)imidazo[1,5-a]pyridin- 6-yl]phenyl]-2- methylpyridine-3- sulfonamide | 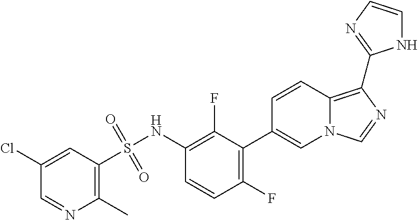 [SMILES: Cc1ncc(Cl)cc1S(=O)(=O)Nc1ccc(F)c(-c2ccc3c(-c4ncc[nH]4)ncn3c2)c1F] | | [M + H]⁺: 501 |
| 26 | 5-chloro-N-[3,5-difluoro- 4-[1-(1H-imidazol-2- yl)imidazo[1,5-a]pyridin- 6-yl]pyridin-2-yl]-2- methoxypyridine-3- sulfonamide | 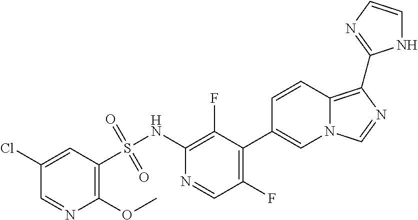 [SMILES: COc1ncc(Cl)cc1S(=O)(=O)Nc1ncc(F)c(-c2ccc3c(-c4ncc[nH]4)ncn3c2)c1F] | 2 | [M + H]⁺: 518 |
| 27-1 | (6R)-6-[3-(5-chloro-2- methoxypyridine-3- sulfonamido)-2,6- difluorophenyl]-N- methyl-5H,6H,7H,8H- imidazo[1,5-a]pyridine- 1-carboxamide | 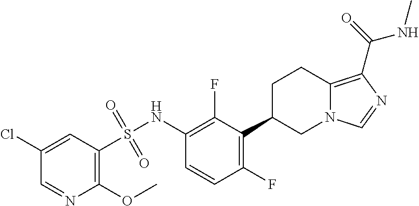 [SMILES: CNC(=O)c1ncn2c1CC[C@H](c1c(F)ccc(NS(=O)(=O)c3cc(Cl)cnc3OC)c1F)C2] | 2, 16 | [M + H]⁺: 512 |
| 27-2 | (6S)-6-[3-(5-chloro-2- methoxypyridine-3- sulfonamido)-2,6- difluorophenyl]-N- methyl-5H,6H,7H,8H- imidazo[1,5-a]pyridine- 1-carboxamide | 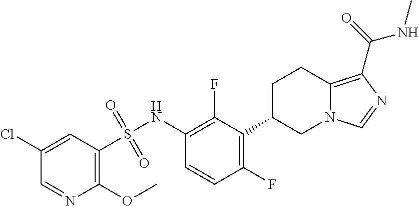 [SMILES: CNC(=O)c1ncn2c1CC[C@@H](c1c(F)ccc(NS(=O)(=O)c3cc(Cl)cnc3OC)c1F)C2] | 2, 16 | [M + H]⁺: 512 |
| 28 | N-[2,4-difluoro-3-[1- (1H-imidazol-2- yl)imidazo[1,5-a]pyridin- 6-yl]phenyl]-6-fluoro-1- hydroxy-2,3-dihydro-1H- indene-4-sulfonamide | 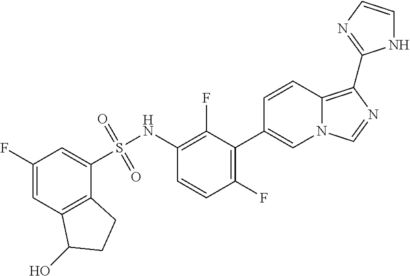 [SMILES: O=S(=O)(Nc1ccc(F)c(-c2ccc3c(-c4ncc[nH]4)ncn3c2)c1F)c1cc(F)cc2c1CCC2O] | | [M + H]⁺: 526 |
| 29 | 5-Chloro-N-[2,4- difluoro-3-[1-(4H-1,2,4- triazol-3-yl)imidazo[1,5- a]pyridin-6-yl]phenyl]-2- methoxypyridine-3- sulfonamide | 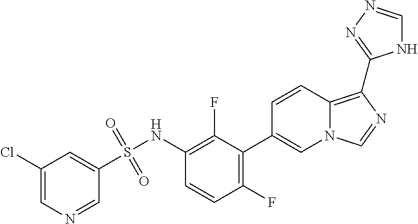 [SMILES: O=S(=O)(Nc1ccc(F)c(-c2ccc3c(-c4nnc[nH]4)ncn3c2)c1F)c1cncc(Cl)c1] | 2, 6 | [M + H]⁺: 518 |
| 30 | 7-[3-(5-chloro-2- methoxypyridine-3- sulfonamido)-2,6- difluorophenyl]-N- methylimidazo[1,5- a]pyridine-3- carboxamide | 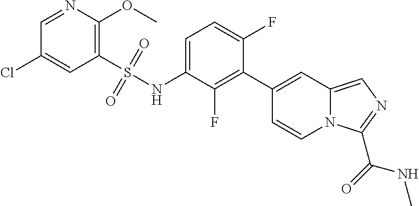 [SMILES: CNC(=O)c1ncc2cc(-c3c(F)ccc(NS(=O)(=O)c4cc(Cl)cnc4OC)c3F)ccn12] | 2 | [M + H]⁺: 508 |
| 31 | 5-Chloro-N-[2,4- difluoro-3-[1-(1H- imidazol-2- yl)imidazo[1,5- a]pyrazin-6-yl]phenyl]-2- methoxypyridine-3- sulfonamide | 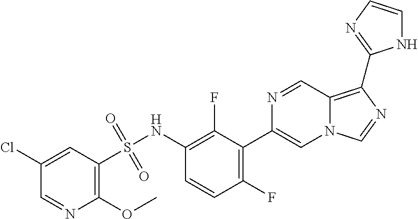 [SMILES: COc1ncc(Cl)cc1S(=O)(=O)Nc1ccc(F)c(-c2cn3cnc(-c4ncc[nH]4)c3cn2)c1F] | 2 | [M + H]⁺: 518 |
| 32 | 5-chloro-N-[2,4-difluoro- 3-[1-(2H-pyrazol-3- yl)imidazo[1,5-a]pyridin- 6-yl]phenyl]-2- methoxypyridine-3- sulfonamide | 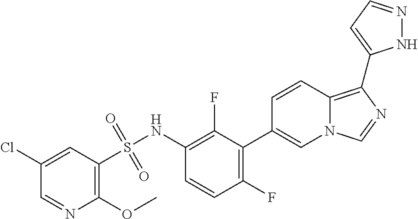 [SMILES: COc1ncc(Cl)cc1S(=O)(=O)Nc1ccc(F)c(-c2ccc3c(-c4ccn[nH]4)ncn3c2)c1F] | 2 | [M + H]⁺: 517 |
| 33-1 | (6R)-6-[3-(5-chloro-2- methoxypyridine-3- sulfonamido)-2,6- difluorophenyl]-N,7- dimethyl-5H,6H,8H- imidazo[1,5-a]pyrazine- 1-carboxamide | 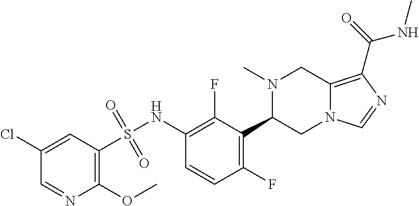 [SMILES: CNC(=O)c1ncn2c1CN(C)[C@H](c1c(F)ccc(NS(=O)(=O)c3cc(Cl)cnc3OC)c1F)C2] | 2 | [M + H]⁺: 527 |
| 33-2 | (6S)-6-[3-(5-chloro-2- methoxypyridine-3- sulfonamido)-2,6- difluorophenyl]-N,7- dimethyl-5H,6H,8H- imidazo[1,5-a]pyrazine- 1-carboxamide | 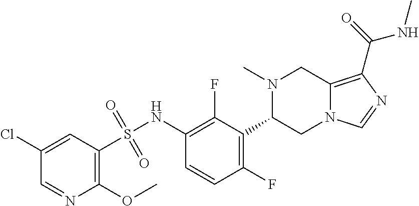 [SMILES: CNC(=O)c1ncn2c1CN(C)[C@@H](c1c(F)ccc(NS(=O)(=O)c3cc(Cl)cnc3OC)c1F)C2] | 2 | [M + H]⁺: 527 |
| 34 | 5-chloro-N-[2,4-difluoro- 3-[1-(3H-1,2,3-triazol-4- yl)imidazo[1,5-a]pyridin- 6-yl]phenyl]-2- methoxypyridine-3- sulfonamide | 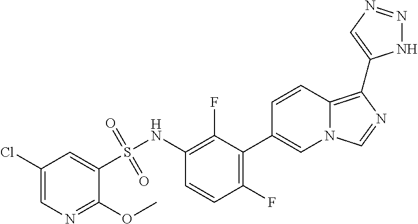 [SMILES: COc1ncc(Cl)cc1S(=O)(=O)Nc1ccc(F)c(-c2ccc3c(-c4cnn[nH]4)ncn3c2)c1F] | 2, 16 | [M + H]⁺: 518 |
| 35 | 5-chloro-N-[2,4-difluoro- 3-[3-(1H-imidazol-2- yl)imidazo[1,5-a]pyridin- 7-yl]phenyl]-2- methoxypyridine-3- sulfonamide | 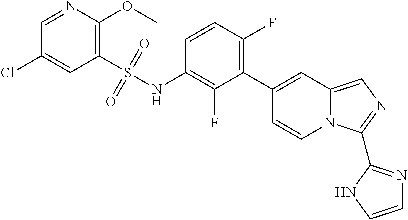 [SMILES: COc1ncc(Cl)cc1S(=O)(=O)Nc1ccc(F)c(-c2ccn3c(-c4ncc[nH]4)ncc3c2)c1F] | 2 | [M + H]⁺: 517 |
| 36 | N-[2,4-difluoro-3-[3- (1H-imidazol-2- yl)imidazo[1,5-a]pyridin- 7-yl]phenyl]-5-fluoro-2- methoxypyridine-3- sulfonamide | 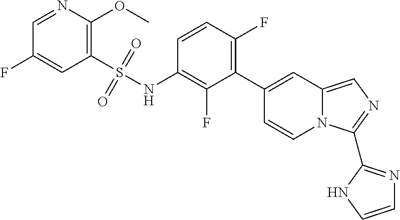 [SMILES: COc1ncc(F)cc1S(=O)(=O)Nc1ccc(F)c(-c2ccn3c(-c4ncc[nH]4)ncc3c2)c1F] | 1 | [M + H]⁺: 501 |
| 37 | 5-chloro-N-[2,4-difluoro- 3-[1-(1-methylpyrazol-3- yl)imidazo[1,5-a]pyridin- 6-yl]phenyl]-2- methoxypyridine-3- sulfonamide | 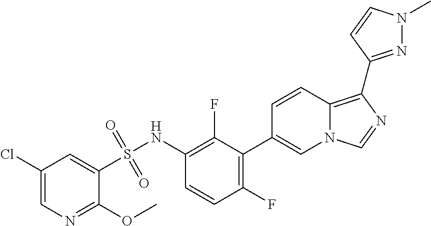 [SMILES: COc1ncc(Cl)cc1S(=O)(=O)Nc1ccc(F)c(-c2ccc3c(-c4ccn(C)n4)ncn3c2)c1F] | 2 | [M + H]⁺: 531 |
| 38 | 5-Chloro-N-[2-cyano-4- fluoro-3-[1-(1H- imidazol-2- yl)imidazo[1,5-a]pyridin- 6-yl]phenyl]-2- methoxypyridine-3- sulfonamide | 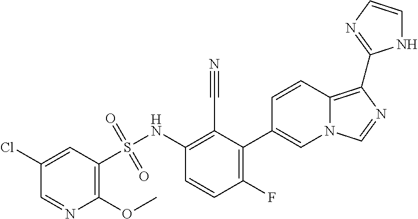 [SMILES: COc1ncc(Cl)cc1S(=O)(=O)Nc1ccc(F)c(-c2ccc3c(-c4ncc[nH]4)ncn3c2)c1C#N] | 2 | [M + H]⁺: 524 |
| 39 | 6-Cyano-N-[2,4-difluoro- 3-[1-(1H-imidazol-2- yl)imidazo[1,5-a]pyridin- 6-yl]phenyl]-1-hydroxy- 2,3-dihydro-1H-indene- 4-sulfonamide | 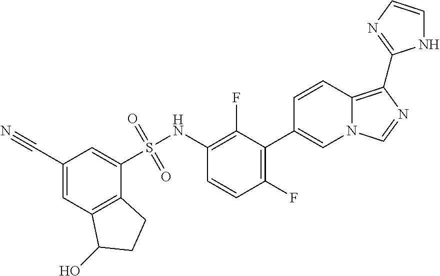 [SMILES: N#Cc1cc2c(c(S(=O)(=O)Nc3ccc(F)c(-c4ccc5c(-c6ncc[nH]6)ncn5c4)c3F)c1)CCC2O] | 8 | [M + H]⁺: 533 |
| 40 | 5-cyano-N-[2,4-difluoro- 3-[3-(1H-imidazol-2- yl)imidazo[1,5-a]pyridin- 7-yl]phenyl]-2- methoxypyridine-3- sulfonamide | 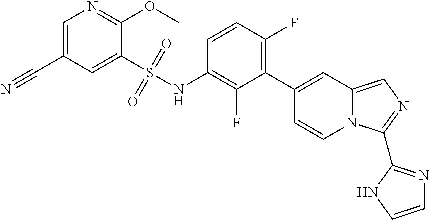 [SMILES: COc1ncc(C#N)cc1S(=O)(=O)Nc1ccc(F)c(-c2ccn3c(-c4ncc[nH]4)ncc3c2)c1F] | 3 | [M + H]⁺: 508 |
| 41 | 5-chloro-N-[2,4-difluoro- 3-[8-fluoro-3-(1H- imidazol-2- yl)imidazo[1,5-a]pyridin- 7-yl]phenyl]-2- methoxypyridine-3- sulfonamide | 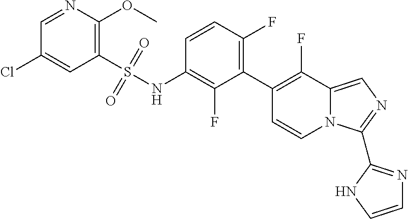 [SMILES: COc1ncc(Cl)cc1S(=O)(=O)Nc1ccc(F)c(-c2ccn3c(-c4ncc[nH]4)ncc3c2F)c1F] | 2 | [M + H]⁺: 535 |
| 42 | 5-cyano-N-[2,4-difluoro- 3-[8-fluoro-3-(1H- imidazol-2- yl)imidazo[1,5-a]pyridin- 7-yl]phenyl]-2- methoxypyridine-3- sulfonamide | 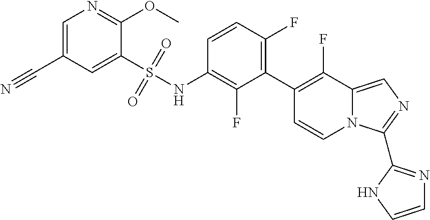 [SMILES: COc1ncc(C#N)cc1S(=O)(=O)Nc1ccc(F)c(-c2ccn3c(-c4ncc[nH]4)ncc3c2F)c1F] | 3 | [M + H]⁺: 526 |
| 43 | 5-chloro-N-(3-(1-(4- cyano-1H-imidazol-2- yl)imidazo[1,5-a]pyridin- 6-yl)-2,4- difluorophenyl)-2- methoxypyridine-3- sulfonamide | 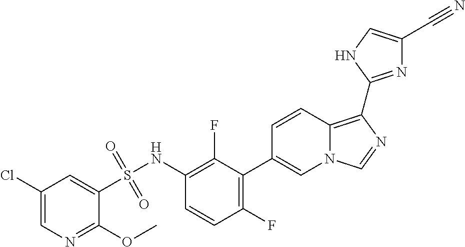 [SMILES: COc1ncc(Cl)cc1S(=O)(=O)Nc1ccc(F)c(-c2ccc3c(-c4nc(C#N)c[nH]4)ncn3c2)c1F] | 2, 7 | [M + H]⁺: 542 |
| 44 | N-[3-[1-(4-cyano-1H- imidazol-2- yl)imidazo[1,5-a]pyridin- 6-yl]-2,4- difluorophenyl]-5-fluoro- 2-methylpyridine-3- sulfonamide | 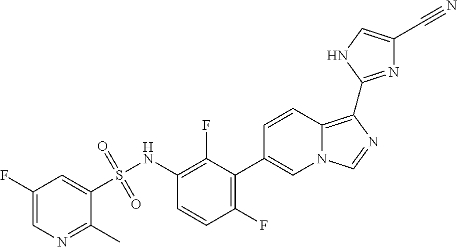 [SMILES: Cc1ncc(F)cc1S(=O)(=O)Nc1ccc(F)c(-c2ccc3c(-c4nc(C#N)c[nH]4)ncn3c2)c1F] | 4 | [M + H]⁺: 510 |
| 45 | N-[3-[1-(4-chloro-1H- imidazol-2- yl)imidazo[1,5-a]pyridin- 6-yl]-2,4- difluorophenyl]-5-fluoro- 2-methoxypyridine-3- sulfonamide | 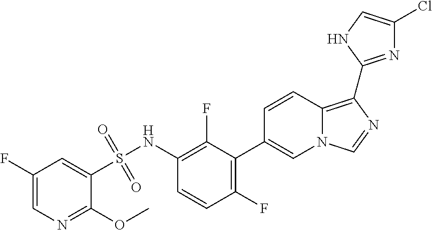 [SMILES: COc1ncc(F)cc1S(=O)(=O)Nc1ccc(F)c(-c2ccc3c(-c4nc(Cl)c[nH]4)ncn3c2)c1F] | 1 | [M + H]⁺: 535 |
| 46 | N-[2,4-difluoro-3-[1-(1- methylpyrazol-3- yl)imidazo[1,5-a]pyridin- 6-yl]phenyl]-5-fluoro-2- methoxypyridine-3- sulfonamide | 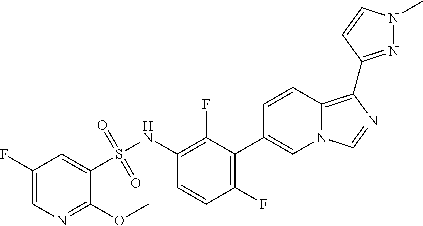 [SMILES: COc1ncc(F)cc1S(=O)(=O)Nc1ccc(F)c(-c2ccc3c(-c4ccn(C)n4)ncn3c2)c1F] | 1 | [M + H]⁺: 515 |
| 47 | N-[2,4-difluoro-3-[8- fluoro-3-(1H-imidazol-2- yl)imidazo[1,5-a]pyridin- 7-yl]phenyl]-5-fluoro-2- methoxypyridine-3- sulfonamide | 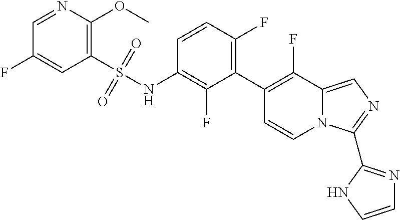 [SMILES: COc1ncc(F)cc1S(=O)(=O)Nc1ccc(F)c(-c2ccn3c(-c4ncc[nH]4)ncc3c2F)c1F] | 1 | [M + H]⁺: 519 |
| 48 | 5-chloro-N-[2,4-difluoro- 3-[1-(4-methyl-3H- imidazol-2- yl)imidazo[1,5-a]pyridin- 6-yl]phenyl]-2- methoxypyridine-3- sulfonamide | 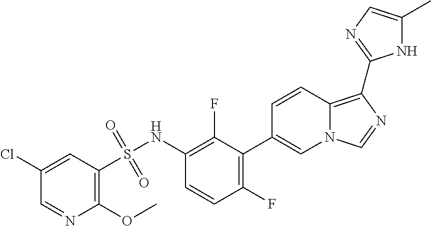 [SMILES: COc1ncc(Cl)cc1S(=O)(=O)Nc1ccc(F)c(-c2ccc3c(-c4ncc(C)[nH]4)ncn3c2)c1F] | 2, 7 | [M + H]⁺: 531 |
| 49 | 5-chloro-N-(2,4-difluoro- 3-[1-[4-(hydroxymethyl)- 3H-imidazol-2- yl]imidazo[1,5-a]pyridin- 6-yl]phenyl)-2- methoxypyridine-3- sulfonamide | 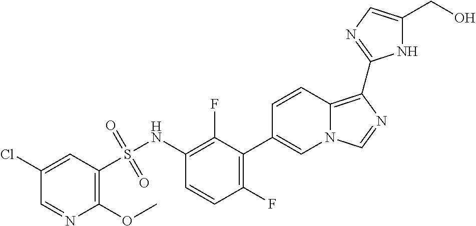 [SMILES: COc1ncc(Cl)cc1S(=O)(=O)Nc1ccc(F)c(-c2ccc3c(-c4ncc(CO)[nH]4)ncn3c2)c1F] | 2, 7 | [M + H]⁺: 547 |
| 50 | N-[2,4-difluoro-3-[1-(4- methyl-3H-imidazol-2- yl)imidazo[1,5-a]pyridin- 6-yl]phenyl]-5-fluoro-2- methylpyridine-3- sulfonamide | 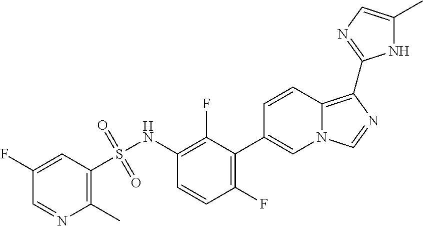 [SMILES: Cc1cnc(-c2ncn3cc(-c4c(F)ccc(NS(=O)(=O)c5cc(F)cnc5C)c4F)ccc23)[nH]1] | 4 | [M + H]⁺: 499 |
| 51 | N-(2,4-difluoro-3-[1-[4- (hydroxymethyl)-3H- imidazol-2- yl]imidazo[1,5-a]pyridin- 6-yl]phenyl)-5-fluoro-2- methylpyridine-3- sulfonamide | 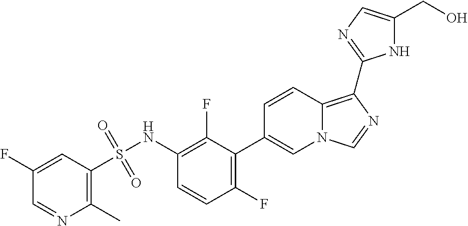 [SMILES: Cc1ncc(F)cc1S(=O)(=O)Nc1ccc(F)c(-c2ccc3c(-c4ncc(CO)[nH]4)ncn3c2)c1F] | 4 | [M + H]⁺: 515 |
| 52 | 5-chloro-N-[3-[1-(4- chloro-1H-imidazol-2- yl)imidazo[1,5-a]pyridin- 6-yl]-2,4- difluorophenyl]-2- methoxypyridine-3- sulfonamide | 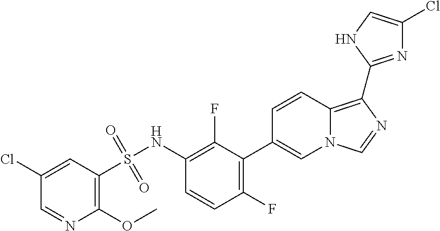 [SMILES: COc1ncc(Cl)cc1S(=O)(=O)Nc1ccc(F)c(-c2ccc3c(-c4nc(Cl)c[nH]4)ncn3c2)c1F] | 2 | [M + H]⁺: 551 |
| 53 | 5-cyano-N-[2,4-difluoro- 3-[1-(1-methylpyrazol-3- yl)imidazo[1,5-a]pyridin- 6-yl]phenyl]-2- methoxypyridine-3- sulfonamide | 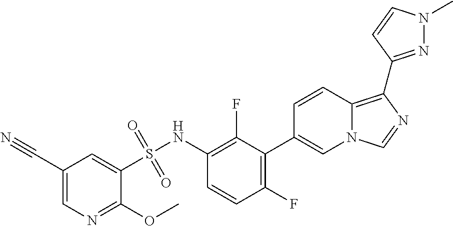 [SMILES: COc1ncc(C#N)cc1S(=O)(=O)Nc1ccc(F)c(-c2ccc3c(-c4ccn(C)n4)ncn3c2)c1F] | 3 | [M + H]⁺: 522 |
| 54 | N-[2,4-difluoro-3-[1-(1- methylpyrazol-3- yl)imidazo[1,5-a]pyridin- 6-yl]phenyl]-5-fluoro-2- methylpyridine-3- sulfonamide | 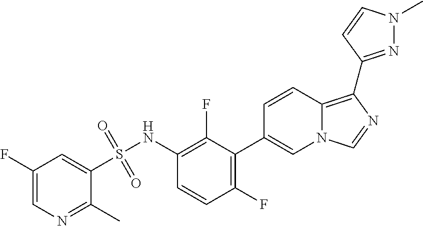 [SMILES: Cc1ncc(F)cc1S(=O)(=O)Nc1ccc(F)c(-c2ccc3c(-c4ccn(C)n4)ncn3c2)c1F] | 4 | [M + H]⁺: 499 |
| 55-1 | 5-chloro-N-[2,4-difluoro- 3-[1-(1,2,3-triazol-1- yl)imidazo[1,5-a]pyridin- 6-yl]phenyl]-2- methoxypyridine-3- sulfonamide | 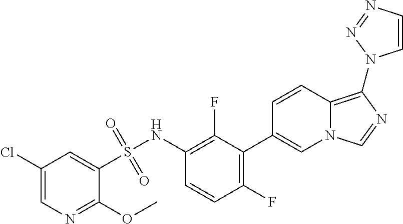 [SMILES: COc1ncc(Cl)cc1S(=O)(=O)Nc1ccc(F)c(-c2ccc3c(-n4ccnn4)ncn3c2)c1F] | 2 | [M + H]⁺: 518 |
| 55-2 | 5-chloro-N-[2,4-difluoro- 3-[1-(1,2,3-triazol-2- yl)imidazo[1,5-a]pyridin- 6-yl]phenyl]-2- methoxypyridine-3- sulfonamide | 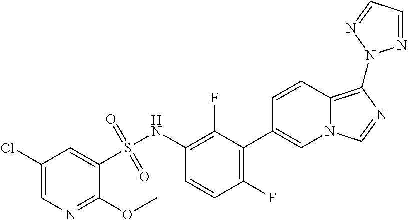 [SMILES: COc1ncc(Cl)cc1S(=O)(=O)Nc1ccc(F)c(-c2ccc3c(-n4nccn4)ncn3c2)c1F] | 2 | [M + H]⁺: 518 |
| 56 | 5-chloro-N-[2,4-difluoro- 3-[1-(4-methyl-3H- imidazol-2- yl)imidazol[1,5-a] pyridin-6-yl]phenyl]-2- methylpyridine-3- sulfonamide | 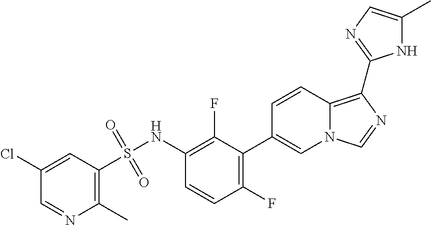 [SMILES: Cc1cnc(-c2ncn3cc(-c4c(F)ccc(NS(=O)(=O)c5cc(Cl)cnc5C)c4F)ccc23)[nH]1] | | [M + H]⁺: 515 |
| 57 | 5-chloro-N-[2,4-difluoro- 3-[1-(5-methyl-4H-1,2,4- triazol-3-yl)imidazo[1,5- a]pyridin-6-yl]phenyl]-2- methoxypyridine-3- sulfonamide | 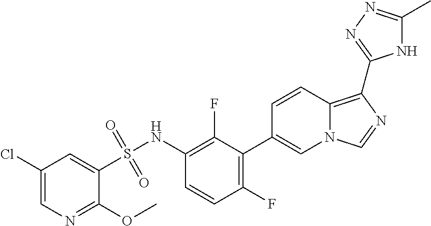 [SMILES: COc1ncc(Cl)cc1S(=O)(=O)Nc1ccc(F)c(-c2ccc3c(-c4nnc(C)[nH]4)ncn3c2)c1F] | 2 | [M + H]⁺: 532 |
| 58 | N-[2,4-difluoro-3-[1-(5- methyl-4H-1,2,4-triazol- 3-yl)imidazo[1,5- a]pyridin-6-yl]phenyl]-5- fluoro-2- methoxypyridine-3- sulfonamide | 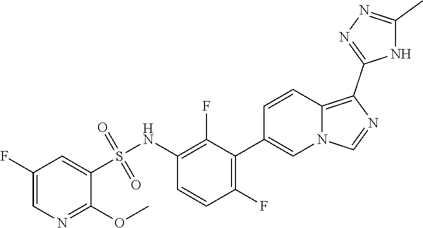 [SMILES: COc1ncc(F)cc1S(=O)(=O)Nc1ccc(F)c(-c2ccc3c(-c4nnc(C)[nH]4)ncn3c2)c1F] | 1 | [M + H]⁺: 516 |
| 59 | 5-cyano-N-[2,4-difluoro- 3-[1-(5-methyl-4H-1,2,4- triazol-3-yl)imidazo[1,5- a]pyridin-6-yl]phenyl]-2- methoxypyridine-3- sulfonamide | 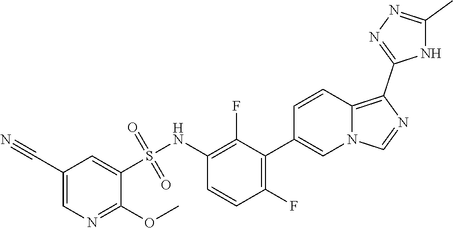 [SMILES: COc1ncc(C#N)cc1S(=O)(=O)Nc1ccc(F)c(-c2ccc3c(-c4nnc(C)[nH]4)ncn3c2)c1F] | 3 | [M + H]⁺: 523 |
| 60 | N-[2,4-difluoro-3-[1-(5- methyl-4H-1,2,4-triazol- 3-yl)imidazo[1,5- a]pyridin-6-yl]phenyl]-5- fluoro-2-methylpyridine- 3-sulfonamide | 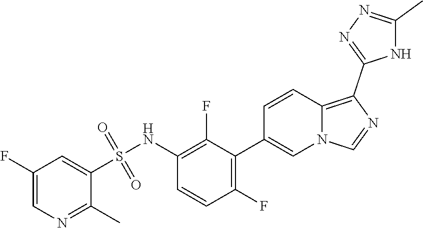 [SMILES: Cc1nnc(-c2ncn3cc(-c4c(F)ccc(NS(=O)(=O)c5cc(F)cnc5C)c4F)ccc23)[nH]1] | 4 | [M + H]⁺: 500 |
| 61 | 5-chloro-N-[2,4-difluoro- 3-[1-(5-methyl-4H-1,2,4- triazol-3-yl)imidazo[1,5- a]pyridin-6-yl]phenyl]-2- methylpyridine-3- sulfonamide | 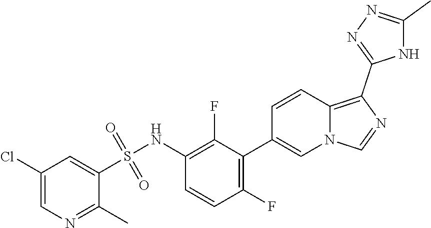 [SMILES: Cc1nnc(-c2ncn3cc(-c4c(F)ccc(NS(=O)(=O)c5cc(Cl)cnc5C)c4F)ccc23)[nH]1] | | [M + H]⁺: 516 |
| 62 | 5-chloro-N-[2,4-difluoro- 3-[1-(1-methylpyrazol-3- yl)imidazo[1,5- a]pyridin-6-yl]phenyl]-2- methylpyridine-3- sulfonamide | 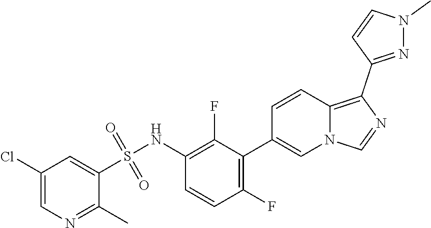 [SMILES: Cc1ncc(Cl)cc1S(=O)(=O)Nc1ccc(F)c(-c2ccc3c(-c4ccn(C)n4)ncn3c2)c1F] | | [M + H]⁺: 515 |
| 63 | 5-chloro-N-[2,4-difluoro- 3-[1-(1,2,3,4-tetrazol-1- yl)imidazo[1,5-a]pyridin- 6-yl]phenyl]-2- methoxypyridine-3- sulfonamide | 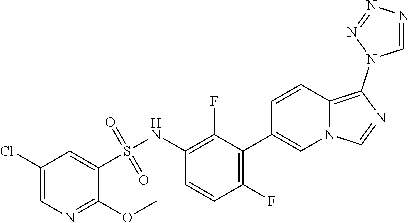 [SMILES: COc1ncc(Cl)cc1S(=O)(=O)Nc1ccc(F)c(-c2ccc3c(-n4cnnn4)ncn3c2)c1F] | 2, 7 | [M + H]⁺: 519 |
| 64-1 | (R)-N-(3-(3-(1H- imidazol-2-yl)-5,6,7,8- tetrahydroimidazo[1,5- a]pyridin-7-yl)-2,4- difluorophenyl)-5- chloro-2-methoxy- pyridine-3-sulfonamide | 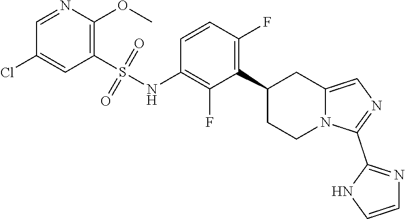 [SMILES: COc1ncc(Cl)cc1S(=O)(=O)Nc1ccc(F)c([C@@H]2CCn3c(cnc3-c3ncc[nH]3)C2)c1F] | 2 | [M + H]⁺: 521 |
| 64-2 | (S)-N-(3-(3-(1H- imidazol-2-yl)-5,6,7,8- tetrahydroimidazo[1,5- a]pyridin-7-yl)-2,4- difluorophenyl)-5- chloro-2-methoxy- pyridine-3- sulfonamide | 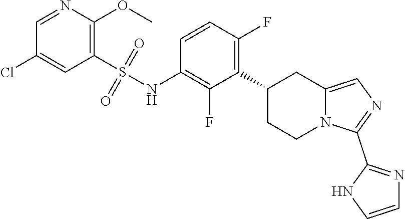 [SMILES: COc1ncc(Cl)cc1S(=O)(=O)Nc1ccc(F)c([C@H]2CCn3c(cnc3-c3ncc[nH]3)C2)c1F] | 2 | [M + H]⁺: 521 |
| 65 | 5-chloro-N-[2,4-difluoro- 3-[1-(4H-1,2,4-triazol-3- yl)imidazo[1,5-a]pyridin- 6-yl]phenyl]-2- methylpyridine-3- sulfonamide | 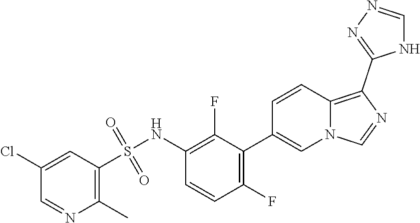 [SMILES: Cc1ncc(Cl)cc1S(=O)(=O)Nc1ccc(F)c(-c2ccc3c(-c4nnc[nH]4)ncn3c2)c1F] | | [M + H]⁺: 502 |
| 66 | N-[3-[1-(1H-1,3- benzodiazol-2- yl)imidazo[1,5-a]pyridin- 6-yl]-2,4- difluorophenyl]-5- cyano-2-methoxy- pyridine-3- sulfonamide | 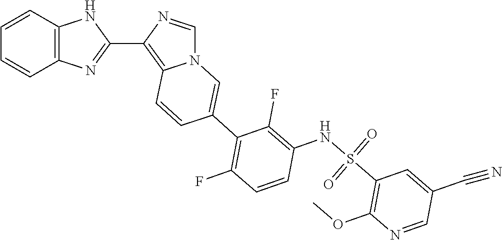 [SMILES: COc1ncc(C#N)cc1S(=O)(=O)Nc1ccc(F)c(-c2ccc3c(-c4nc5ccccc5[nH]4)ncn3c2)c1F] | 3 | [M + H]⁺: 558 |
| 67-1 | (R)-6-(2,6-difluoro-3-((5- fluoro-2- methoxypyridine)-3- sulfonamido)phenyl)-N- methyl-5,6,7,8- tetrahydroimidazo[1,5- a]pyridine-1- carboxamide | 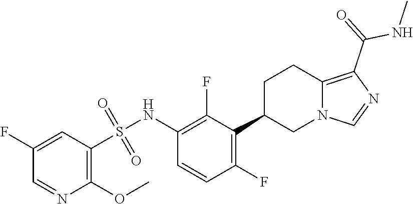 [SMILES: CNC(=O)c1ncn2c1CC[C@H](c1c(F)ccc(NS(=O)(=O)c3cc(F)cnc3OC)c1F)C2] | 1 | [M + H]⁺: 496 |
| 67-2 | (S)-6-(2,6-difluoro-3-((5- fluoro-2- methoxypyridine)-3- sulfonamido)phenyl)-N- methyl-5,6,7,8- tetrahydroimidazo[1,5- a]pyridine-1- carboxamide | 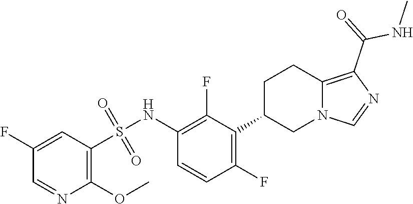 [SMILES: CNC(=O)c1ncn2c1CC[C@@H](c1c(F)ccc(NS(=O)(=O)c3cc(F)cnc3OC)c1F)C2] | 1 | [M + H]⁺: 496 |
| 68-1 | (6R)-6-[3-(5-cyano-2- methoxypyridine-3- sulfonamido)-2,6- difluorophenyl]-N- methyl-5H,6H,7H,8H- imidazo[1,5-a]pyridine- 1-carboxamide | 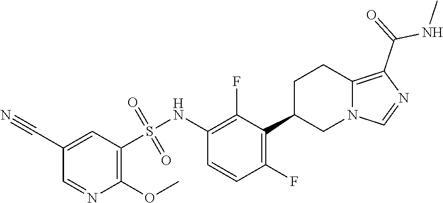 [SMILES: CNC(=O)c1ncn2c1CC[C@H](c1c(F)ccc(NS(=O)(=O)c3cc(C#N)cnc3OC)c1F)C2] | 3 | [M + H]⁺: 503 |
| 68-2 | (6S)-6-[3-(5-cyano-2- methoxypyridine-3- sulfonamido)-2,6- difluorophenyl]-N- methyl-5H,6H,7H,8H- imidazo[1,5-a]pyridine- 1-carboxamide | 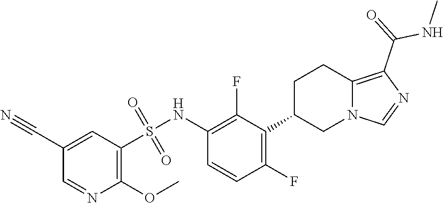 [SMILES: CNC(=O)c1ncn2c1CC[C@@H](c1c(F)ccc(NS(=O)(=O)c3cc(C#N)cnc3OC)c1F)C2] | 3 | [M + H]⁺: 503 |
| 69 | 5-cyano-N-[2,4-difluoro- 3-[1-(1H-imidazol-2- yl)imidazo[1,5-a] pyrazin-6-yl]phenyl]-2- methoxypyridine-3- sulfonamide | 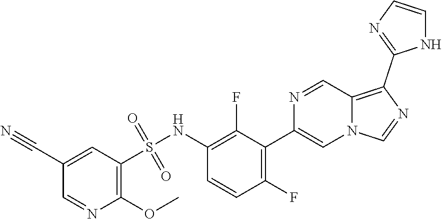 [SMILES: COc1ncc(C#N)cc1S(=O)(=O)Nc1ccc(F)c(-c2cn3cnc(-c4ncc[nH]4)c3cn2)c1F] | 3 | [M + H]⁺: 509 |
| 70 | N-[2,4-difluoro-3-[1- (4H-1,2,4-triazol-3- yl)imidazo[1,5-a]pyridin- 6-yl]phenyl]-5-fluoro-2- methoxypyridine-3- sulfonamide | 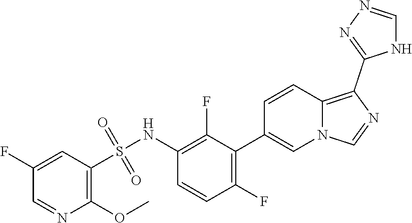 [SMILES: COc1ncc(F)cc1S(=O)(=O)Nc1ccc(F)c(-c2ccc3c(-c4nnc[nH]4)ncn3c2)c1F] | 1 | [M + H]⁺: 502 |
| 71 | 5-cyano-N-[2,4-difluoro- 3-[1-(4H-1,2,4-triazol-3- yl)imidazo[1,5-a]pyridin- 6-yl]phenyl]-2- methoxypyridine-3- sulfonamide | 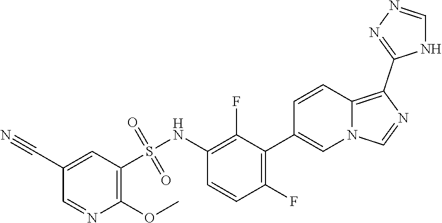 [SMILES: COc1ncc(C#N)cc1S(=O)(=O)Nc1ccc(F)c(-c2ccc3c(-c4nnc[nH]4)ncn3c2)c1F] | 3 | [M + H]⁺: 509 |
| 72 | N-[2,4-difluoro-3-[1- (4H-1,2,4-triazol-3- yl)imidazo[1,5-a]pyridin- 6-yl]phenyl]-5-fluoro-2- methylpyridine-3- sulfonamide | 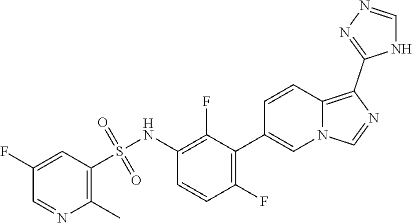 [SMILES: Cc1ncc(F)cc1S(=O)(=O)Nc1ccc(F)c(-c2ccc3c(-c4nnc[nH]4)ncn3c2)c1F] | 4 | [M + H]⁺: 486 |
| 73-1 | 5-chloro-N-[2,4-difluoro- 3-[(6R)-1-(1H-imidazol- 2-yl)-7-methyl- 5H,6H,8H-imidazo[1,5- a]pyrazin-6-yl]phenyl]-2- methoxypyridine-3- sulfonamide | 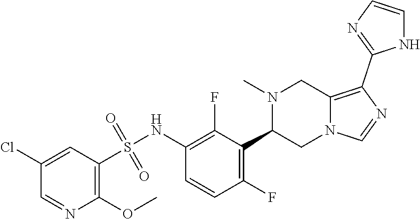 [SMILES: COc1ncc(Cl)cc1S(=O)(=O)Nc1ccc(F)c([C@@H]2Cn3cnc(-c4ncc[nH]4)c3CN2C)c1F] | 2 | [M + H]⁺: 536 |
| 73-2 | 5-chloro-N-[2,4-difluoro- 3-[(6S)-1-(1H-imidazol- 2-yl)-7-methyl- 5H,6H,8H-imidazo[1,5- a]pyrazin-6-yl]phenyl]-2- methoxypyridine-3- sulfonamide | 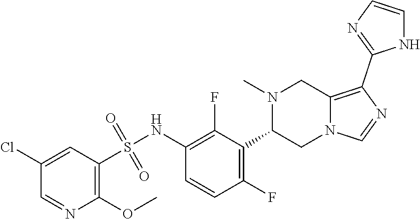 [SMILES: COc1ncc(Cl)cc1S(=O)(=O)Nc1ccc(F)c([C@H]2Cn3cnc(-c4ncc[nH]4)c3CN2C)c1F] | 2 | [M + H]⁺: 536 |
| 74 | N-[2,4-difluoro-3-[1- (1,2,3,4-tetrazol-1- yl)imidazo[1,5-a]pyridin- 6-yl]phenyl]-5-fluoro-2- methoxypyridine-3- sulfonamide | 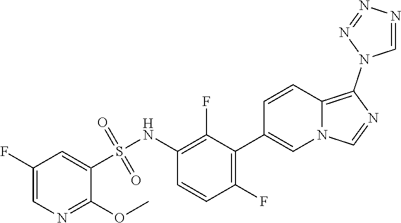 [SMILES: COc1ncc(F)cc1S(=O)(=O)Nc1ccc(F)c(-c2ccc3c(-n4cnnn4)ncn3c2)c1F] | 1 | [M + H]⁺: 503 |
| 75 | 5-cyano-N-[2,4-difluoro- 3-[1-(1,2,3,4-tetrazol-1- yl)imidazo[1,5- a]pyridin-6-yl]phenyl]-2- methoxypyridine-3- sulfonamide | 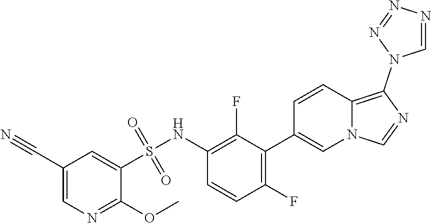 [SMILES: COc1ncc(C#N)cc1S(=O)(=O)Nc1ccc(F)c(-c2ccc3c(-n4cnnn4)ncn3c2)c1F] | 3 | [M + H]⁺: 510 |
| 76 | N-[2,4-difluoro-3-[1- (1,2,3,4-tetrazol-1- yl)imidazo[1,5-a]pyridin- 6-yl]phenyl]-5-fluoro-2- methylpyridine-3- sulfonamide | 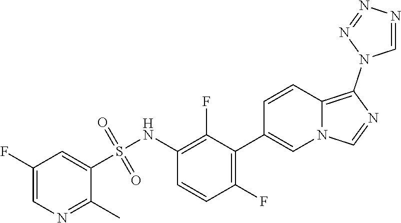 [SMILES: Cc1ncc(F)cc1S(=O)(=O)Nc1ccc(F)c(-c2ccc3c(-n4cnnn4)ncn3c2)c1F] | 4 | [M + H]⁺: 487 |
| 77 | 5-chloro-N-[2,4-difluoro- 3-[1-(1,2,3,4-tetrazol-1- yl)imidazo[1,5-a]pyridin- 6-yl]phenyl]-2- methylpyridine-3- sulfonamide | 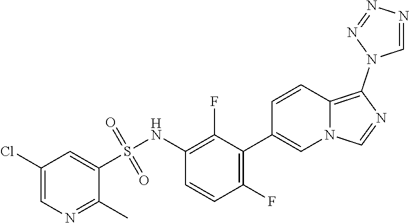 [SMILES: Cc1ncc(Cl)cc1S(=O)(=O)Nc1ccc(F)c(-c2ccc3c(-n4cnnn4)ncn3c2)c1F] | | [M + H]⁺: 503 |
| 78 | N-[2,4-difluoro-3-[1- (1H-imidazol-2- yl)imidazo[1,5- a]pyrazin-6-yl]phenyl]-5- fluoro-2- methoxypyridine-3- sulfonamide | 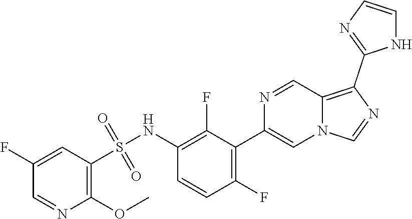 [SMILES: COc1ncc(F)cc1S(=O)(=O)Nc1ccc(F)c(-c2cn3cnc(-c4ncc[nH]4)c3cn2)c1F] | 1 | [M + H]⁺: 502 |
| 79-1 | 5-chloro-N-[2,4-difluoro- 3-[(6R)-1-(1H-imidazol- 2-yl)-5H,6H,7H,8H- imidazo[1,5-a]pyridin-6- yl]phenyl]-2- methoxypyridine-3- sulfonamide | 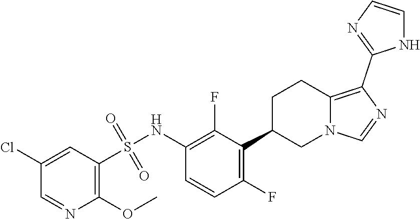 [SMILES: COc1ncc(Cl)cc1S(=O)(=O)Nc1ccc(F)c([C@H]2CCc3c(-c4ncc[nH]4)ncn3C2)c1F] | 2 | [M + H]⁺: 521 |
| 79-2 | 5-chloro-N-[2,4-difluoro- 3-[(6S)-1-(1H-imidazol- 2-yl)-5H,6H,7H,8H- imidazo[1,5-a]pyridin-6- yl]phenyl]-2- methoxypyridine-3- sulfonamide | 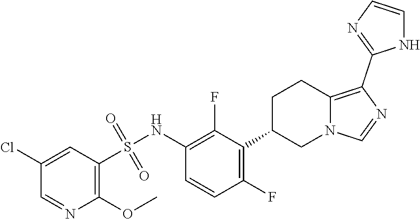 [SMILES: COc1ncc(Cl)cc1S(=O)(=O)Nc1ccc(F)c([C@@H]2CCc3c(-c4ncc[nH]4)ncn3C2)c1F] | 2 | [M + H]⁺: 521 |
| 80-1 | N-[2,4-difluoro-3-[(6R)- 1-(1H-imidazol-2-yl)- 5H,6H,7H,8H- imidazo[1,5-a]pyridin-6- yl]phenyl]-5-fluoro-2- methyl pyridine-3- sulfonamide | 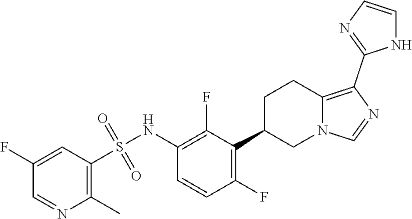 [SMILES: Cc1ncc(F)cc1S(=O)(=O)Nc1ccc(F)c([C@H]2CCc3c(-c4ncc[nH]4)ncn3C2)c1F] | 4 | [M + H]⁺: 489 |
| 80-2 | N-[2,4-difluoro-3-[(6S)- 1-(1H-imidazol-2-yl)- 5H,6H,7H,8H- imidazo[1,5-a]pyridin-6- yl]phenyl]-5-fluoro-2- methyl pyridine-3- sulfonamide | 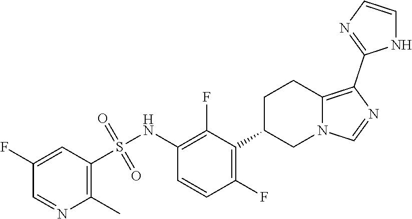 [SMILES: Cc1ncc(F)cc1S(=O)(=O)Nc1ccc(F)c([C@@H]2CCc3c(-c4ncc[nH]4)ncn3C2)c1F] | 4 | [M + H]⁺: 489 |
| 81-1 | N-[2,4-difluoro-3-[(6R)- 1-(1H-imidazol-2-yl)- 5H,6H,7H,8H- imidazo[1,5-a]pyridin-6- yl]phenyl]-5-fluoro-2- methoxypyridine-3- sulfonamide | 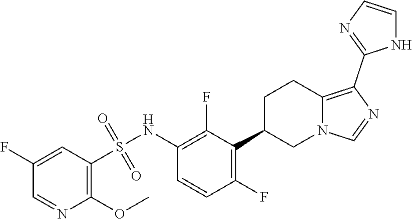 [SMILES: COc1ncc(F)cc1S(=O)(=O)Nc1ccc(F)c([C@H]2CCc3c(-c4ncc[nH]4)ncn3C2)c1F] | 1 | [M + H]⁺: 505 |
| 81-2 | N-[2,4-difluoro-3-[(6S)- 1-(1H-imidazol-2-yl)- 5H,6H,7H,8H- imidazo[1,5-a]pyridin-6- yl]phenyl]-5-fluoro-2- methoxypyridine-3- sulfonamide | 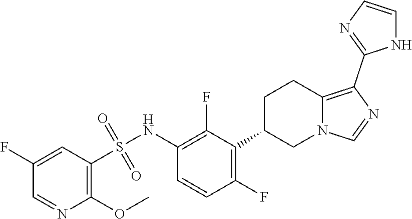 [SMILES: COc1ncc(F)cc1S(=O)(=O)Nc1ccc(F)c([C@@H]2CCc3c(-c4ncc[nH]4)ncn3C2)c1F] | 1 | [M + H]⁺: 505 |
| 82-1 | 5-cyano-N-[2,4-difluoro- 3-[(6R)-1-(1H-imidazol- 2-yl)-5H,6H,7H,8H- imidazo[1,5-a]pyridin-6- yl]phenyl]-2- methoxypyridine-3- sulfonamide | 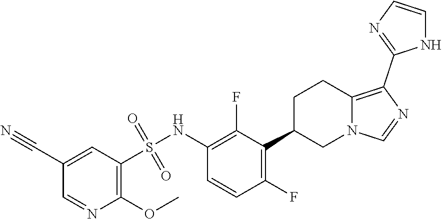 [SMILES: COc1ncc(C#N)cc1S(=O)(=O)Nc1ccc(F)c([C@H]2CCc3c(-c4ncc[nH]4)ncn3C2)c1F] | 3 | [M + H]⁺: 512 |
| 82-2 | 5-cyano-N-[2,4-difluoro- 3-[(6S)-1-(1H-imidazol- 2-yl)-5H,6H,7H,8H- imidazo[1,5-a]pyridin-6- yl]phenyl]-2- methoxypyridine-3- sulfonamide | 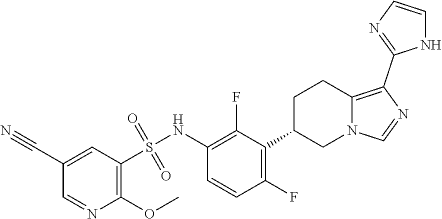 [SMILES: COc1ncc(C#N)cc1S(=O)(=O)Nc1ccc(F)c([C@@H]2CCc3c(-c4ncc[nH]4)ncn3C2)c1F] | 3 | [M + H]⁺: 512 |
| 83 | 5-chloro-N-[2,4-difluoro- 3-[1-(1H-imidazol-2- yl)imidazo[1,5-a] pyrazin-6-yl]phenyl]- 2-methylpyridine-3- sulfonamide | 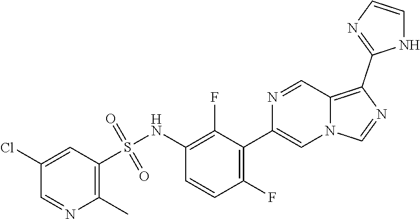 [SMILES: Cc1ncc(Cl)cc1S(=O)(=O)Nc1ccc(F)c(-c2cn3cnc(-c4ncc[nH]4)c3cn2)c1F] | | [M + H]⁺: 502 |
| 84-1 | 5-chloro-N-[2,4-difluoro- 3-[(6R)-1-(1H-imidazol- 2-yl)-5H,6H,7H,8H- imidazo[1,5-a]pyridin-6- yl]phenyl]-2- methylpyridine-3- sulfonamide | 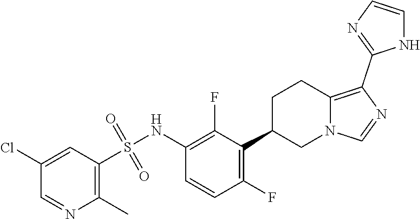 [SMILES: Cc1ncc(Cl)cc1S(=O)(=O)Nc1ccc(F)c([C@H]2CCc3c(-c4ncc[nH]4)ncn3C2)c1F] | | [M + H]⁺: 505 |
| 84-2 | 5-chloro-N-[2,4-difluoro- 3-[(6S)-1-(1H-imidazol- 2-yl)-5H,6H,7H,8H- imidazo[1,5-a]pyridin-6- yl]phenyl]-2- methylpyridine-3- sulfonamide | 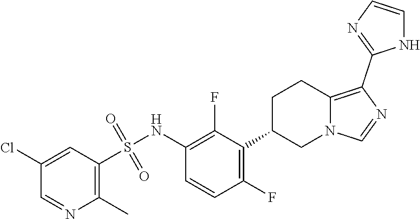 [SMILES: Cc1ncc(Cl)cc1S(=O)(=O)Nc1ccc(F)c([C@@H]2CCc3c(-c4ncc[nH]4)ncn3C2)c1F] | | [M + H]⁺: 505 |
| 85 | 5-chloro-N-[2,4-difluoro- 3-[5-(1H-imidazol-2- yl)imidazo[1,5- b]pyridazin-2-yl] phenyl]-2-methoxy- pyridine-3- sulfonamide | 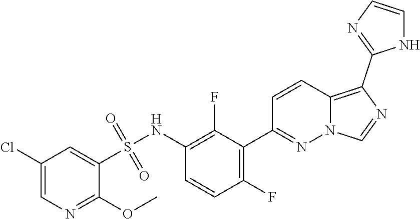 [SMILES: COc1ncc(Cl)cc1S(=O)(=O)Nc1ccc(F)c(-c2ccc3c(-c4ncc[nH]4)ncn3n2)c1F] | 2 | [M + H]⁺: 518 |
| 86-1 | (R)-6-(2,6-difluoro-3-((5- fluoro-2- methylpyridine)-3- sulfonamido)phenyl)-N- methyl-5,6,7,8- tetrahydroimidazo[1,5- a]pyridine-1- carboxamide | 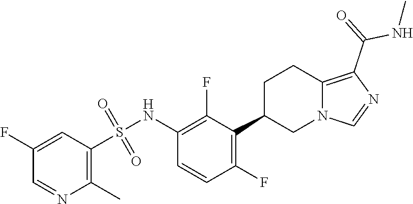 [SMILES: CNC(=O)c1ncn2c1CC[C@H](c1c(F)ccc(NS(=O)(=O)c3cc(F)cnc3C)c1F)C2] | 4 | [M + H]⁺: 480 |
| 86-2 | (S)-6-(2,6-difluoro-3-((5- fluoro-2- methylpyridine)-3- sulfonamido)phenyl)-N- methyl-5,6,7,8- tetrahydroimidazo[1,5- a]pyridine-1- carboxamide | 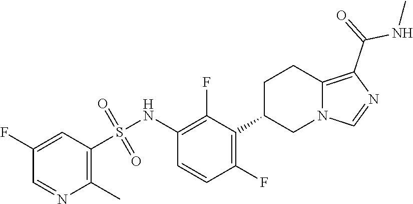 [SMILES: CNC(=O)c1ncn2c1CC[C@@H](c1c(F)ccc(NS(=O)(=O)c3cc(F)cnc3C)c1F)C2] | 4 | [M + H]⁺: 480 |
| 87 | 5-chloro-N-[3-[1-(5- cyclopropyl-4H-1,2,4- triazol-3-yl)imidazo[1,5- a]pyridin-6-yl]-2,4- difluorophenyl]-2- methoxypyridine-3- sulfonamide | 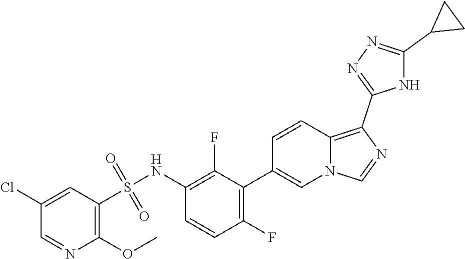 [SMILES: COc1ncc(Cl)cc1S(=O)(=O)Nc1ccc(F)c(-c2ccc3c(-c4nnc(C5CC5)[nH]4)ncn3c2)c1F] | 2 | [M + H]⁺: 558 |
| 88 | 5-chloro-N-[2,4-difluoro- 3-[1-(5-isopropyl-4H- 1,2,4-triazol-3- yl)imidazo[1,5-a]pyridin- 6-yl]phenyl]-2-methoxy- pyridine-3-sulfonamide | 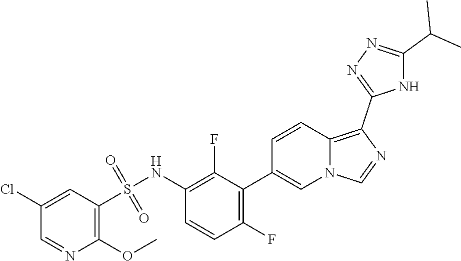 [SMILES: COc1ncc(Cl)cc1S(=O)(=O)Nc1ccc(F)c(-c2ccc3c(-c4nnc(C(C)C)[nH]4)ncn3c2)c1F] | 13, 2 | [M + H]⁺: 560 |
| 89-1 | (R)-6-(3-((5-chloro-2- methylpyridine)-3- sulfonamido)-2,6- difluorophenyl)-N- methyl-5,6,7,8-tetrahydro imidazo [1,5-a]pyridine-1- carboxamide | 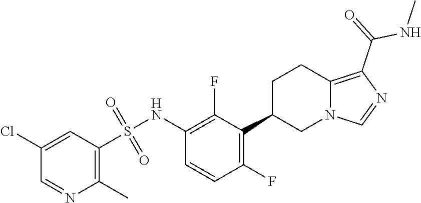 [SMILES: CNC(=O)c1ncn2c1CC[C@H](c1c(F)ccc(NS(=O)(=O)c3cc(Cl)cnc3C)c1F)C2] | | [M + H]⁺: 496 |
| 89-2 | (S)-6-(3-((5-chloro-2- methylpyridine)-3- sulfonamido)-2,6- difluorophenyl)-N- methyl-5,6,7,8-tetrahydro imidazo[1,5-a]pyridine- 1-carboxamide | 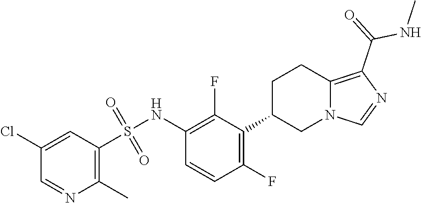 [SMILES: CNC(=O)c1ncn2c1CC[C@@H](c1c(F)ccc(NS(=O)(=O)c3cc(Cl)cnc3C)c1F)C2] | | [M + H]⁺: 496 |
| 90 | 6-[2,6-difluoro-3-(5- fluoro-2- methoxypyridine-3- sulfonamido)phenyl]-N- methylimidazo[1,5- a]pyrazine-1- carboxamide | 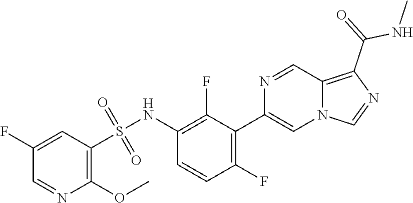 [SMILES: CNC(=O)c1ncn2cc(-c3c(F)ccc(NS(=O)(=O)c4cc(F)cnc4OC)c3F)ncc12] | 1 | [M + H]⁺: 493 |
| 91 | 6-[2,6-difluoro-3-(5- fluoro-2-methylpyridine- 3-sulfonamido)phenyl]- N-methylimidazo[1,5- a]pyrazine-1- carboxamide | 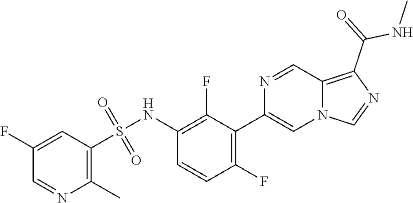 [SMILES: CNC(=O)c1ncn2cc(-c3c(F)ccc(NS(=O)(=O)c4cc(F)cnc4C)c3F)ncc12] | 4 | [M + H]⁺: 477 |
| 92 | 6-[3-(5-cyano-2- methoxypyridine-3- sulfonamido)-2,6- difluorophenyl]-N- methylimidazo[1,5- a]pyrazine-1- carboxamide | 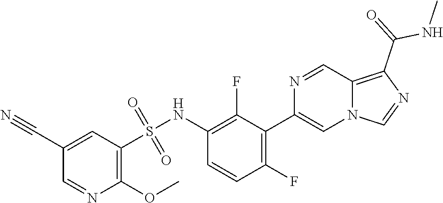 [SMILES: CNC(=O)c1ncn2cc(-c3c(F)ccc(NS(=O)(=O)c4cc(C#N)cnc4OC)c3F)ncc12] | 3 | [M + H]⁺: 500 |
| 93 | 6-[3-(5-chloro-2- methylpyridine-3- sulfonamido)-2,6- difluorophenyl]-N- methylimidazo[1,5- a]pyrazine-1- carboxamide | 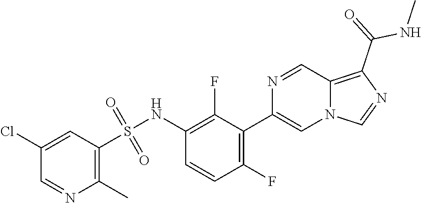 [SMILES: CNC(=O)c1ncn2cc(-c3c(F)ccc(NS(=O)(=O)c4cc(Cl)cnc4C)c3F)ncc12] | | [M + H]⁺: 493 |
| 94 | 5-chloro-N-[2,4-difluoro- 3-[1- (hydroxymethyl)imidazo [1,5-a]pyridin-6- yl]phenyl]-2- methoxypyridine-3- sulfonamide | 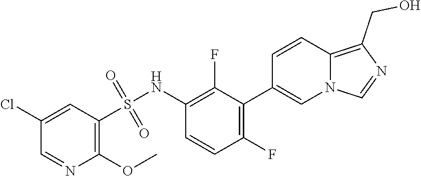 [SMILES: COc1ncc(Cl)cc1S(=O)(=O)Nc1ccc(F)c(-c2ccc3c(CO)ncn3c2)c1F] | 2, 14 | [M + H]⁺: 481 |
| 95 | 5-chloro-N-[2,4-difluoro- 3-[5-(1H-imidazol-2- yl)imidazo[1,5- b]pyridazin-2-yl]phenyl]- 2-methylpyridine-3- sulfonamide | 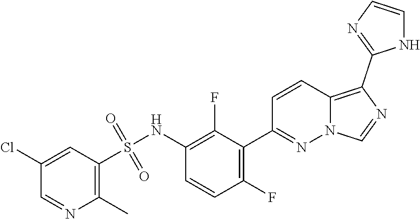 [SMILES: Cc1ncc(Cl)cc1S(=O)(=O)Nc1ccc(F)c(-c2ccc3c(-c4ncc[nH]4)ncn3n2)c1F] | | [M + H]⁺: 502 |
| 96-1 | (5S,6R)-6-[3-(5-chloro- 2-methoxypyridine-3- sulfonamido)-2,6- difluorophenyl]-N,5- dimethyl-5H,6H,7H,8H- imidazo[1,5-a]pyridine- 1-carboxamide | 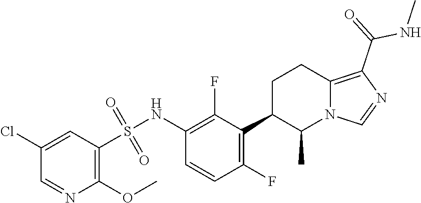 [SMILES: CNC(=O)c1ncn2c1CC[C@H](c1c(F)ccc(NS(=O)(=O)c3cc(Cl)cnc3OC)c1F)[C@@H]2C] | 2 | [M + H]⁺: 526 |
| 96-2 | (5R,6S)-6-[3-(5-chloro- 2-methoxypyridine-3- sulfonamido)-2,6- difluorophenyl]-N,5- dimethyl-5H,6H,7H,8H- imidazo[1,5-a]pyridine- 1-carboxamide | 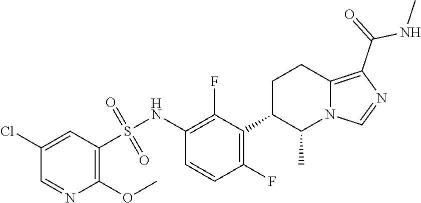 [SMILES: CNC(=O)c1ncn2c1CC[C@@H](c1c(F)ccc(NS(=O)(=O)c3cc(Cl)cnc3OC)c1F)[C@H]2C] | 2 | [M + H]⁺: 526 |
| 97 | 5-cyano-N-[2,4-difluoro- 3-[5-(1H-imidazol-2- yl)imidazo[1,5- b]pyridazin-2-yl]phenyl]- 2-methoxypyridine-3- sulfonamide | 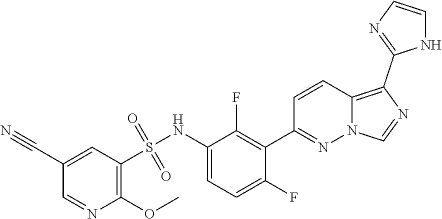 [SMILES: COc1ncc(C#N)cc1S(=O)(=O)Nc1ccc(F)c(-c2ccc3c(-c4ncc[nH]4)ncn3n2)c1F] | 3 | [M + H]⁺: 509 |
| 98 | N-[2,4-difluoro-3-[5- (1H-imidazol-2- yl)imidazo[1,5- b]pyridazin-2- yl]phenyl]-5-fluoro-2- methoxypyridine-3- sulfonamide | 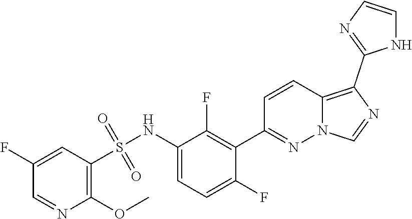 [SMILES: COc1ncc(F)cc1S(=O)(=O)Nc1ccc(F)c(-c2ccc3c(-c4ncc[nH]4)ncn3n2)c1F] | 1 | [M + H]⁺: 502 |
| 99 | 5-chloro-N-(2,4-difluoro- 3-(1-isoxazol-5- yl)imidazo[1,5-a]pyridin- 6-yl)phenyl)-2- methoxypyridine-3- sulfonamide | 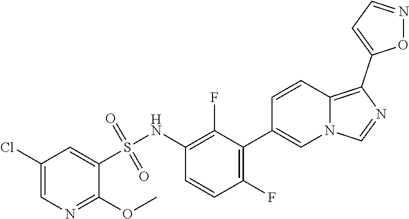 [SMILES: COc1ncc(Cl)cc1S(=O)(=O)Nc1ccc(F)c(-c2ccc3c(-c4ccno4)ncn3c2)c1F] | 2, 13 | [M + H]⁺: 518 |
| 100 | 5-chloro-N-[2,4-difluoro- 3-[1-(1- hydroxyethyl)imidazo [1,5-a]pyridin-6-yl] phenyl]-2- methoxypyridine-3- sulfonamide | 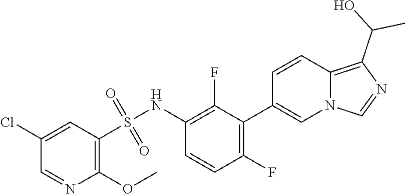 [SMILES: COc1ncc(Cl)cc1S(=O)(=O)Nc1ccc(F)c(-c2ccc3c(C(C)O)ncn3c2)c1F] | | [M + H]⁺: 495 |
| 101 | 6-[2-chloro-3-(5-chloro- 2-methoxypyridine-3- sulfonamido)-6- fluorophenyl]-N- methylimidazo[1,5-a] pyridine-1-carboxamide | 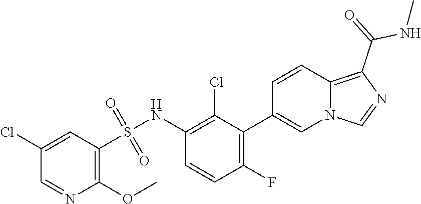 [SMILES: CNC(=O)c1ncn2cc(-c3c(F)ccc(NS(=O)(=O)c4cc(Cl)cnc4OC)c3Cl)ccc12] | 2, 14-a | [M + H]⁺: 524 |
| 102 | 5-chloro-N-(2,4-difluoro- 3-[1-[5-(trifluoromethyl)- 4H-1,2,4-triazol-3- yl]imidazo[1,5-a]pyridin- 6-yl]phenyl)-2- methoxypyridine-3- sulfonamide | 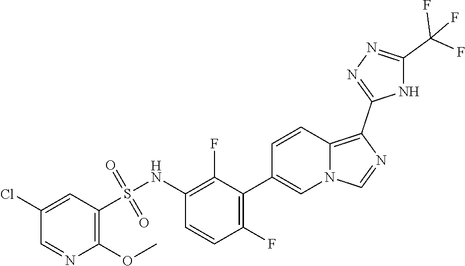 [SMILES: COc1ncc(Cl)cc1S(=O)(=O)Nc1ccc(F)c(-c2ccc3c(-c4nnc(C(F)(F)F)[nH]4)ncn3c2)c1F] | 2 | [M + H]⁺: 586 |
| 103 | 5-chloro-N-[2-chloro-4- fluoro-3-[1-(1H- imidazol-2- yl)imidazo[1,5-a]pyridin- 6-yl]phenyl]-2- methoxypyridine-3- sulfonamide | 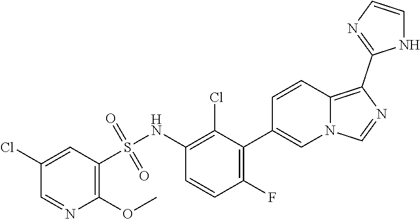 [SMILES: COc1ncc(Cl)cc1S(=O)(=O)Nc1ccc(F)c(-c2ccc3c(-c4ncc[nH]4)ncn3c2)c1Cl] | 2 | [M + H]⁺: 533 |
| 104 | 5-chloro-N-[2,4-difluoro- 3-[5-fluoro-1-(1H- imidazol-2- yl)imidazo[1,5-a]pyridin- 6-yl]phenyl]-2- methoxypyridine-3- sulfonamide | 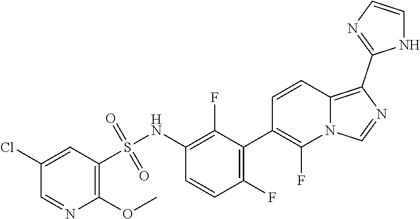 [SMILES: COc1ncc(Cl)cc1S(=O)(=O)Nc1ccc(F)c(-c2ccc3c(-c4ncc[nH]4)ncn3c2F)c1F] | 2 | [M + H]⁺: 535 |
| 105 | 5-chloro-N-[2,4-difluoro- 3-[5-fluoro-1-(2H- pyrazol-3- yl)imidazo[1,5-a]pyridin- 6-yl]phenyl]-2- methoxypyridine-3- sulfonamide | 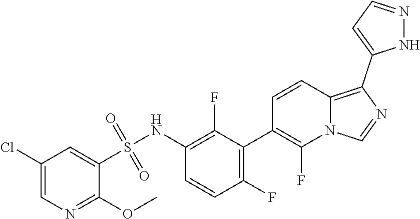 [SMILES: COc1ncc(Cl)cc1S(=O)(=O)Nc1ccc(F)c(-c2ccc3c(-c4ccn[nH]4)ncn3c2F)c1F] | 2 | [M + H]⁺: 535 |
| 106 | 6-[2-chloro-3-(5-chloro- 2-methoxypyridine-3- sulfonamido)-6- fluorophenyl]-N- methylimidazo[1,5- a]pyrazine-1- carboxamide | 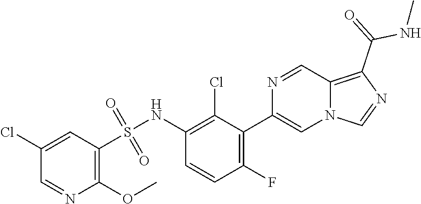 [SMILES: CNC(=O)c1ncn2cc(-c3c(F)ccc(NS(=O)(=O)c4cc(Cl)cnc4OC)c3Cl)ncc12] | 2 | [M + H]⁺: 525 |
| 107 | N-[2,4-difluoro-3-[5- fluoro-1-(1H-imidazol-2- yl)imidazo[1,5-a]pyridin- 6-yl]phenyl]-5-fluoro-2- methoxypyridine-3- sulfonamide | 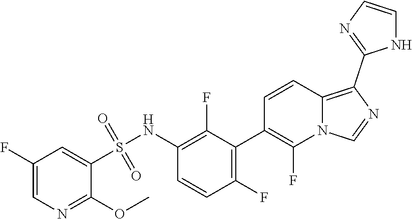 [SMILES: COc1ncc(F)cc1S(=O)(=O)Nc1ccc(F)c(-c2ccc3c(-c4ncc[nH]4)ncn3c2F)c1F] | 1 | [M + H]⁺: 519 |
| 108 | N-[2,4-difluoro-3-[5- fluoro-1-(1H-imidazol-2- yl)imidazo[1,5-a]pyridin- 6-yl]phenyl]-5-fluoro-2- methylpyridine-3- sulfonamide | 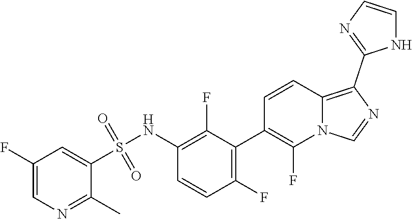 [SMILES: Cc1ncc(F)cc1S(=O)(=O)Nc1ccc(F)c(-c2ccc3c(-c4ncc[nH]4)ncn3c2F)c1F] | 4 | [M + H]⁺: 503 |
| 109 | 5-chloro-N-[2,4-difluoro- 3-[5-fluoro-1-(1H- imidazol-2- yl)imidazo[1,5-a]pyridin- 6-yl]phenyl]-2- methylpyridine-3- sulfonamide | 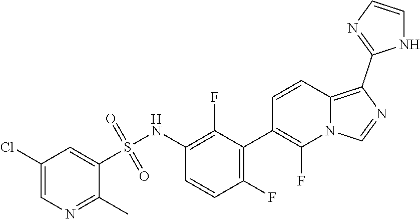 [SMILES: Cc1ncc(Cl)cc1S(=O)(=O)Nc1ccc(F)c(-c2ccc3c(-c4ncc[nH]4)ncn3c2F)c1F] | | [M + H]⁺: 519 |
| 110 | N-[3-[1-(1H-1,3- benzodiazol-2- yl)imidazo[1,5- a]pyrazin-6-yl]-2,4- difluorophenyl]-5- chloro-2-methoxy- pyridine-3-sulfonamide | 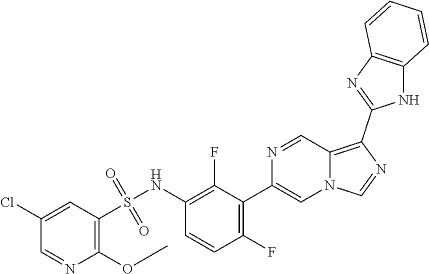 [SMILES: COc1ncc(Cl)cc1S(=O)(=O)Nc1ccc(F)c(-c2cn3cnc(-c4nc5ccccc5[nH]4)c3cn2)c1F] | 2, 11 | [M + H]⁺: 568 |
| 111 | N-[3-[1-(1H-1,3- benzodiazol-2- yl)imidazo[1,5-a] pyrazin-6-yl]-2,4- difluorophenyl]-5-fluoro- 2-methoxypyridine-3- sulfonamide | 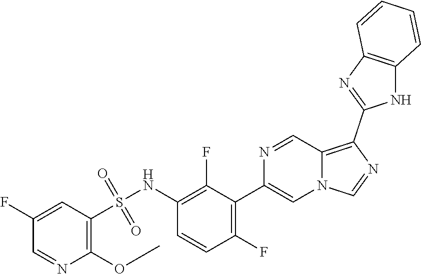 [SMILES: COc1ncc(F)cc1S(=O)(=O)Nc1ccc(F)c(-c2cn3cnc(-c4nc5ccccc5[nH]4)c3cn2)c1F] | 1 | [M + H]⁺: 552 |
| 112 | N-[3-[1-(1H-1,3- benzodiazol-2- yl)imidazo[1,5-a] pyrazin-6-yl]-2,4- difluorophenyl]-5-cyano- 2-methoxypyridine-3- sulfonamide | 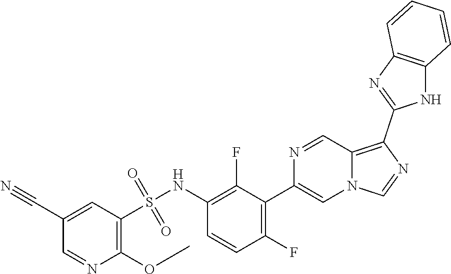 [SMILES: COc1ncc(C#N)cc1S(=O)(=O)Nc1ccc(F)c(-c2cn3cnc(-c4nc5ccccc5[nH]4)c3cn2)c1F] | 3 | [M + H]⁺: 559 |
| 113 | N-[3-[1-(1H-1,3- benzodiazol-2- yl)imidazo[1,5- a]pyrazin-6-yl]-2,4- difluorophenyl]-5- chloro-2-methylpyridine- 3-sulfonamide | 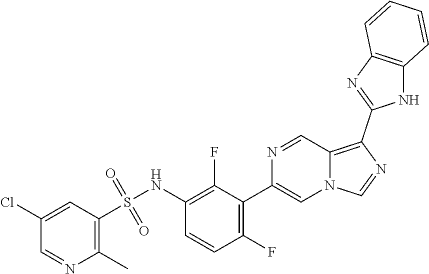 [SMILES: Cc1ncc(Cl)cc1S(=O)(=O)Nc1ccc(F)c(-c2cn3cnc(-c4nc5ccccc5[nH]4)c3cn2)c1F] | | [M + H]⁺: 552 |
| 114 | N-[3-[1-(1H-1,3- benzodiazol-2- yl)imidazo[1,5- a]pyrazin-6-yl]-2,4- difluorophenyl]-5- fluoro-2-methyl- pyridine-3-sulfonamide | 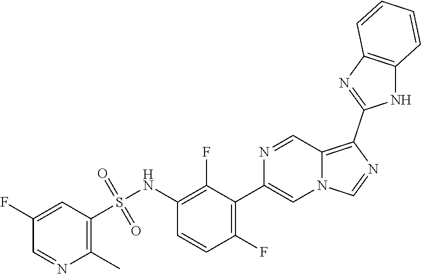 [SMILES: Cc1ncc(F)cc1S(=O)(=O)Nc1ccc(F)c(-c2cn3cnc(-c4nc5ccccc5[nH]4)c3cn2)c1F] | 4 | [M + H]⁺: 536 |
| 115 | N-[3-[1-(4-chloro-1H- imidazol-2-yl)-5- fluoroimidazo[1,5- a]pyridin-6-yl]-2,4- difluorophenyl]-5- fluoro-2-methoxy- pyridine-3-sulfonamide | 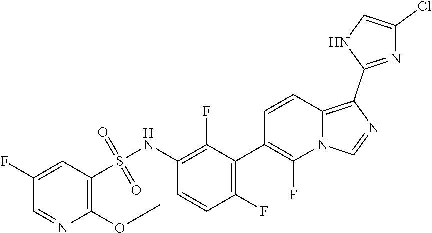 [SMILES: COc1ncc(F)cc1S(=O)(=O)Nc1ccc(F)c(-c2ccc3c(-c4nc(Cl)c[nH]4)ncn3c2F)c1F] | 1 | [M + H]⁺: 553 |
| 116 | 5-chloro-N-[3-[1-(4- chloro-1H-imidazol-2- yl)-5-fluoroimidazo[1,5- a]pyridin-6-yl]-2,4- difluorophenyl]-2- methoxypyridine-3- sulfonamide | 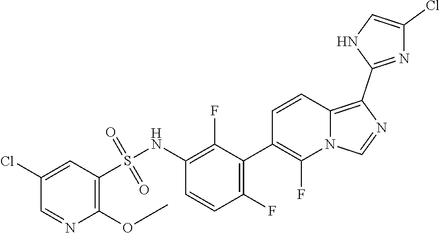 [SMILES: COc1ncc(Cl)cc1S(=O)(=O)Nc1ccc(F)c(-c2ccc3c(-c4nc(Cl)c[nH]4)ncn3c2F)c1F] | 2 | [M + H]⁺: 569 |
| 117 | 6-[3-(5-chloro-2- methoxypyridine-3- sulfonamido)-2-cyano-6- fluorophenyl]-N- methylimidazo[1,5- a]pyrazine-1- carboxamide | 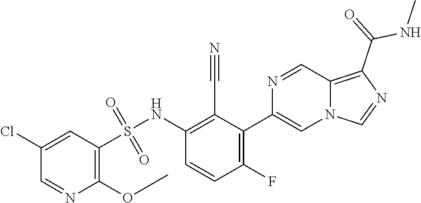 [SMILES: CNC(=O)c1ncn2cc(-c3c(F)ccc(NS(=O)(=O)c4cc(Cl)cnc4OC)c3C#N)ncc12] | | [M + H]⁺: 516 |
| 118 | N-[2,4-difluoro-3-[1- (1,2-oxazol-5- yl)imidazo[1,5-a]pyridin- 6-yl]phenyl]-5-fluoro-2- methoxypyridine-3- sulfonamide | 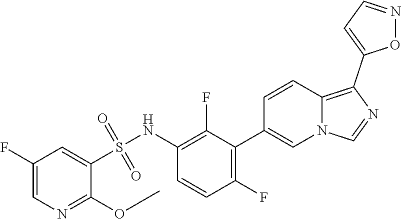 [SMILES: COc1ncc(F)cc1S(=O)(=O)Nc1ccc(F)c(-c2ccc3c(-c4ccno4)ncn3c2)c1F] | 1 | [M + H]⁺: 502 |
| 119 | 5-cyano-N-[2,4-difluoro- 3-[1-(1,2-oxazol-5- yl)imidazo[1,5-a]pyridin- 6-yl]phenyl]-2- methoxypyridine-3- sulfonamide | 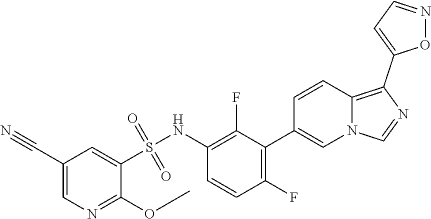 [SMILES: COc1ncc(C#N)cc1S(=O)(=O)Nc1ccc(F)c(-c2ccc3c(-c4ccno4)ncn3c2)c1F] | 3 | [M + H]⁺: 509 |
| 120 | 2-[3-(5-chloro-2- methoxypyridine-3- sulfonamido)-2,6- difluorophenyl]-N- methylimidazo[4,3- b][1,3]thiazole-7- carboxamide | 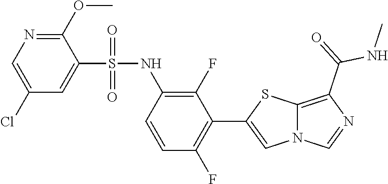 [SMILES: CNC(=O)c1ncn2cc(-c3c(F)ccc(NS(=O)(=O)c4cc(Cl)cnc4OC)c3F)sc12] | 2 | [M + H]⁺: 514 |
| 121 | 5-chloro-N-[2,4-difluoro- 3-[1-(1,2-oxazol-5- yl)imidazo[1,5-a]pyridin- 6-yl]phenyl]-2- methylpyridine-3- sulfonamide | 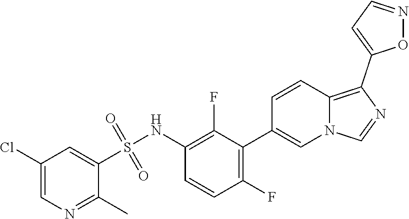 [SMILES: Cc1ncc(Cl)cc1S(=O)(=O)Nc1ccc(F)c(-c2ccc3c(-c4ccno4)ncn3c2)c1F] | | [M + H]⁺: 502 |
| 122 | 6-[3-(5-chloro-2- methoxypyridine-3- sulfonamido)-6-fluoro-2- methylphenyl]-N- methylimidazo[1,5- a]pyridine-1- carboxamide | 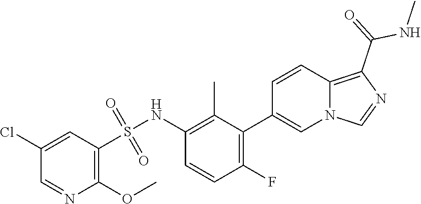 [SMILES: CNC(=O)c1ncn2cc(-c3c(F)ccc(NS(=O)(=O)c4cc(Cl)cnc4OC)c3C)ccc12] | 2, 14-a | [M + H]⁺: 504 |
| 123 | 5-cyano-N-[2,4-difluoro- 3-[1-(4,5,6,7-tetrahydro- 1H-1,3-benzodiazol-2- yl)imidazo[1,5-a]pyridin- 6-yl]phenyl]-2- methoxypyridine-3- sulfonamide | 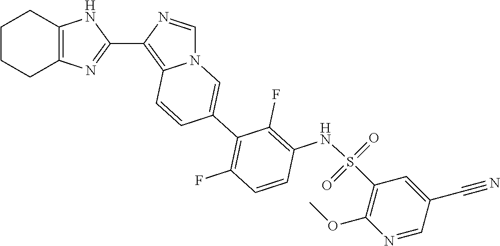 [SMILES: COc1ncc(C#N)cc1S(=O)(=O)Nc1ccc(F)c(-c2ccc3c(-c4nc5c([nH]4)CCCC5)ncn3c2)c1F] | 3 | [M + H]⁺: 562 |
| 124 | 5-chloro-N-[2,4-difluoro- 3-[1-(5-methyl-4H-1,2,4- triazol-3-yl)imidazo[1,5- a]pyrazin-6-yl]phenyl]-2- methoxypyridine-3- sulfonamide | 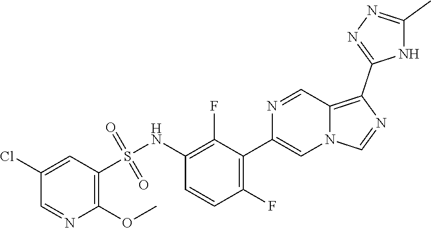 [SMILES: COc1ncc(Cl)cc1S(=O)(=O)Nc1ccc(F)c(-c2cn3cnc(-c4nnc(C)[nH]4)c3cn2)c1F] | 2, 12 | [M + H]⁺: 533 |
| 125 | N-[2,4-difluoro-3-[1- (1,2-oxazol-5- yl)imidazo[1,5-a]pyridin- 6-yl]phenyl]-5-fluoro-2- methylpyridine-3- sulfonamide | 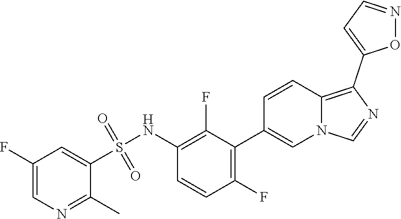 [SMILES: Cc1ncc(F)cc1S(=O)(=O)Nc1ccc(F)c(-c2ccc3c(-c4ccno4)ncn3c2)c1F] | 4 | [M + H]⁺: 486 |
| 126 | 5-cyano-N-[3-[1-(4,5- dimethyl-1H-imidazol- 2-yl)imidazo[1,5- a]pyridin-6-yl]-2,4- difluorophenyl]-2- methoxypyridine-3- sulfonamide | 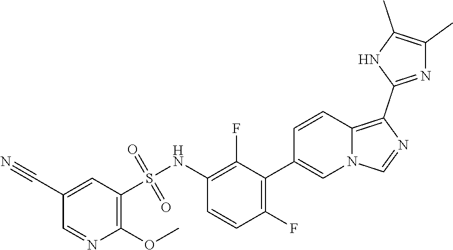 [SMILES: COc1ncc(C#N)cc1S(=O)(=O)Nc1ccc(F)c(-c2ccc3c(-c4nc(C)c(C)[nH]4)ncn3c2)c1F] | 3 | [M + H]⁺: 536 |
| 127 | N-[2,4-difluoro-3-[1-(5- methyl-4H-1,2,4-triazol- 3-yl)imidazo[1,5- a]pyrazin-6-yl]phenyl]-5- fluoro-2- methoxypyridine-3- sulfonamide | 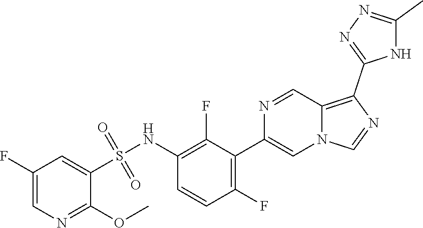 [SMILES: COc1ncc(F)cc1S(=O)(=O)Nc1ccc(F)c(-c2cn3cnc(-c4nnc(C)[nH]4)c3cn2)c1F] | 1 | [M + H]⁺: 517 |
| 128 | 5-cyano-N-[2,4-difluoro- 3-[1-(5-methyl-4H-1,2,4- triazol-3-yl)imidazo[1,5- a]pyrazin-6-yl]phenyl]-2- methoxypyridine-3- carboxamide | 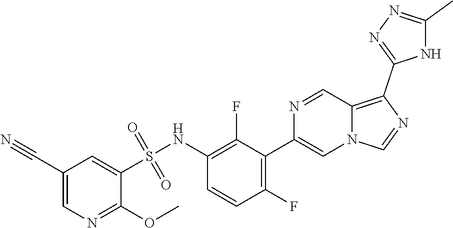 [SMILES: COc1ncc(C#N)cc1S(=O)(=O)Nc1ccc(F)c(-c2cn3cnc(-c4nnc(C)[nH]4)c3cn2)c1F] | 3 | [M + H]⁺: 524 |
| 129 | N-[2,4-difluoro-3-[1-(5- methyl-4H-1,2,4-triazol- 3-yl)imidazo[1,5- a]pyrazin-6-yl]phenyl]-5- fluoro-2-methylpyridine- 3-sulfonamide | 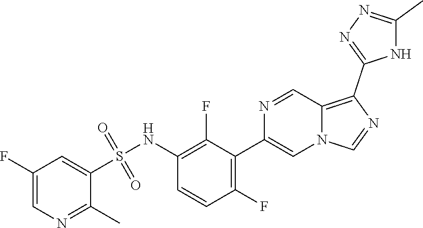 [SMILES: Cc1nnc(-c2ncn3cc(-c4c(F)ccc(NS(=O)(=O)c5cc(F)cnc5C)c4F)ncc23)[nH]1] | 4 | [M + H]⁺: 501 |
| 130-1 | 5-chloro-N-[2,4-difluoro- 3-[(6S)-1-(5-methyl-4H- 1,2,4-triazol-3-yl)- 5H,6H,7H,8H- imidazo[1,5-a]pyridin-6- yl]phenyl]-2- methoxypyridine-3- sulfonamide | 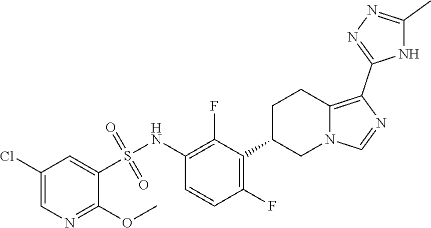 [SMILES: COc1ncc(Cl)cc1S(=O)(=O)Nc1ccc(F)c([C@@H]2CCc3c(-c4nnc(C)[nH]4)ncn3C2)c1F] | 2 | [M + H]⁺: 536 |
| 130-2 | 5-chloro-N-[2,4-difluoro- 3-[(6R)-1-(5-methyl-4H- 1,2,4-triazol-3-yl)- 5H,6H,7H,8H- imidazo[1,5-a]pyridin-6- yl]phenyl]-2- methoxypyridine-3- sulfonamide | 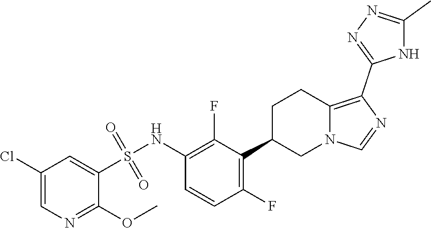 [SMILES: COc1ncc(Cl)cc1S(=O)(=O)Nc1ccc(F)c([C@H]2CCc3c(-c4nnc(C)[nH]4)ncn3C2)c1F] | 2 | [M + H]⁺: 536 |
| 131-1 | N-[2,4-difluoro-3-[(6S)- 1-(5-methyl-4H-1,2,4- triazol-3-yl)- 5H,6H,7H,8H- imidazo[1,5-a]pyridin-6- yl]phenyl]-5-fluoro-2- methoxypyridine-3- sulfonamide | 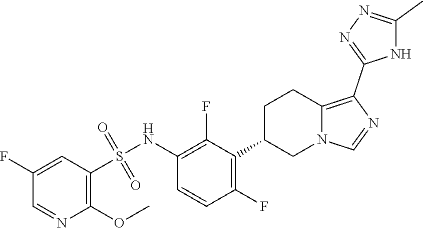 [SMILES: COc1ncc(F)cc1S(=O)(=O)Nc1ccc(F)c([C@@H]2CCc3c(-c4nnc(C)[nH]4)ncn3C2)c1F] | 1 | [M + H]⁺: 520 |
| 131-2 | N-[2,4-difluoro-3-[(6R)- 1-(5-methyl-4H-1,2,4- triazol-3-yl)- 5H,6H,7H,8H- imidazo[1,5-a]pyridin-6- yl]phenyl]-5-fluoro-2- methoxypyridine-3- sulfonamide | 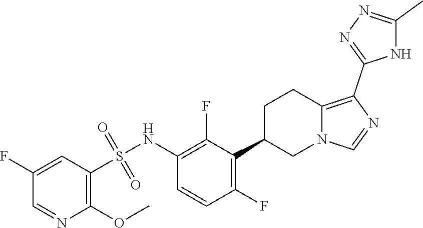 [SMILES: COc1ncc(F)cc1S(=O)(=O)Nc1ccc(F)c([C@H]2CCc3c(-c4nnc(C)[nH]4)ncn3C2)c1F] | 1 | [M + H]⁺: 520 |
| 132 | 6-(3-((5-chloro-2-oxo- 1,2-dihydropyridine)-3- sulfonamido)-2,6- difluorophenyl)-N- methylimidazo[1,5- a]pyrazine-1- carboxamide | 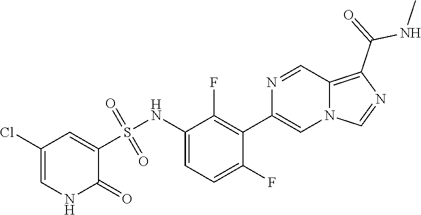 [SMILES: CNC(=O)c1ncn2cc(-c3c(F)ccc(NS(=O)(=O)c4cc(Cl)c[nH]c4=O)c3F)ncc12] | | [M + H]⁺: 495 |
| 133 | 5-chloro-N-[2,4-difluoro- 3-[1-(5-methyl-4H-1,2,4- triazol-3-yl)imidazo[1,5- a]pyrazin-6-yl]phenyl]-2- methylpyridine-3- sulfonamide | 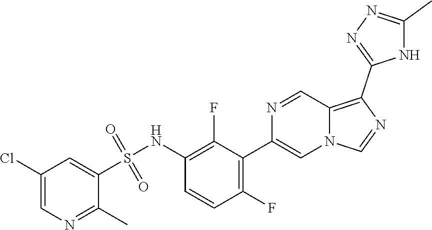 [SMILES: Cc1nnc(-c2ncn3cc(-c4c(F)ccc(NS(=O)(=O)c5cc(Cl)cnc5C)c4F)ncc23)[nH]1] | | [M + H]⁺: 517 |
| 134-1 | N-[2,4-difluoro-3-[(6S)- 1-(5-methyl-4H-1,2,4- triazol-3-yl)- 5H,6H,7H,8H- imidazo[1,5-a]pyridin-6- yl]phenyl]-5-fluoro-2- methylpyridine-3- sulfonamide | 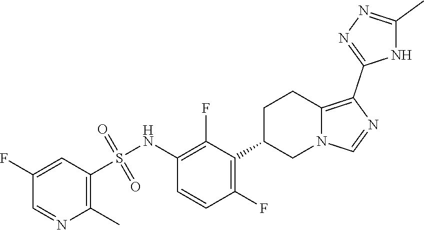 [SMILES: Cc1nnc(-c2ncn3c2CC[C@@H](c2c(F)ccc(NS(=O)(=O)c4cc(F)cnc4C)c2F)C3)[nH]1] | 4 | [M + H]⁺: 504 |
| 134-2 | N-[2,4-difluoro-3-[(6R)- 1-(5-methyl-4H-1,2,4- triazol-3-yl)- 5H,6H,7H,8H- imidazo[1,5-a]pyridin-6- yl]phenyl]-5-fluoro-2- methylpyridine-3- sulfonamide | 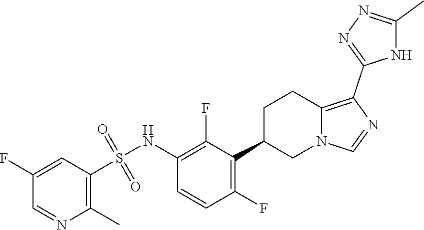 [SMILES: Cc1nnc(-c2ncn3c2CC[C@H](c2c(F)ccc(NS(=O)(=O)c4cc(F)cnc4C)c2F)C3)[nH]1] | 4 | [M + H]⁺: 504 |
| 135-1 | 5-cyano-N-[2,4-difluoro- 3-[(6S)-1-(5-methyl-4H- 1,2,4-triazol-3-yl)- 5H,6H,7H,8H- imidazo[1,5-a]pyridin-6- yl]phenyl]-2- methoxypyridine-3- sulfonamide | 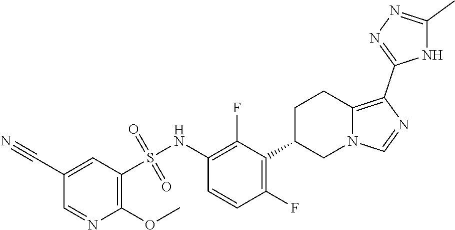 [SMILES: COc1ncc(C#N)cc1S(=O)(=O)Nc1ccc(F)c([C@@H]2CCc3c(-c4nnc(C)[nH]4)ncn3C2)c1F] | 3 | [M + H]⁺: 527 |
| 135-2 | 5-cyano-N-[2,4-difluoro- 3-[(6R)-1-(5-methyl-4H- 1,2,4-triazol-3-yl)- 5H,6H,7H,8H- imidazo[1,5-a]pyridin-6- yl]phenyl]-2- methoxypyridine-3- sulfonamide [M + H]⁺: 527 | 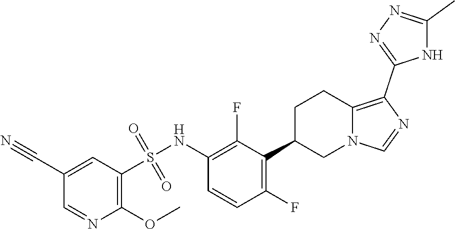 [SMILES: COc1ncc(C#N)cc1S(=O)(=O)Nc1ccc(F)c([C@H]2CCc3c(-c4nnc(C)[nH]4)ncn3C2)c1F] | 3 | [M + H]⁺: 527 |
| 136-1 | 5-chloro-N-[2,4-difluoro- 3-[(6S)-1-(5-methyl-4H- 1,2,4-triazol-3-yl)- 5H,6H,7H,8H- imidazo[1,5-a]pyridin-6- yl]phenyl]-2- methylpyridine-3- sulfonamide | 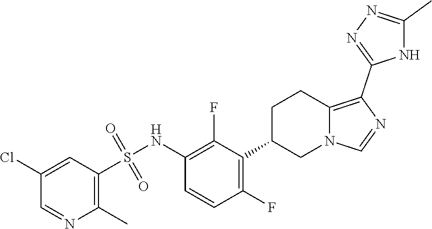 [SMILES: Cc1nnc(-c2ncn3c2CC[C@@H](c2c(F)ccc(NS(=O)(=O)c4cc(Cl)cnc4C)c2F)C3)[nH]1] | | [M + H]⁺: 520 |
| 136-2 | 5-chloro-N-[2,4-difluoro- 3-[(6R)-1-(5-methyl-4H- 1,2,4-triazol-3-yl)- 5H,6H,7H,8H- imidazo[1,5-a]pyridin-6- yl]phenyl]-2- methylpyridine-3- sulfonamide | 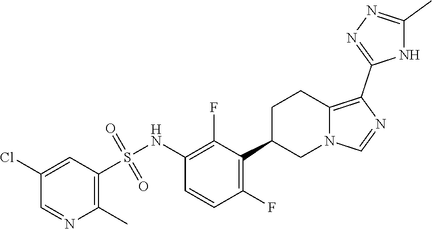 [SMILES: Cc1nnc(-c2ncn3c2CC[C@H](c2c(F)ccc(NS(=O)(=O)c4cc(Cl)cnc4C)c2F)C3)[nH]1] | | [M + H]⁺: 520 |
| 137 | 6-[6-chloro-3-(5-chloro- 2-methoxypyridine-3- sulfonamido)-2- fluorophenyl]-N- methylimidazo[1,5- a]pyridine-1- carboxamide | 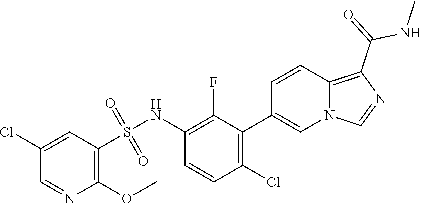 [SMILES: CNC(=O)c1ncn2cc(-c3c(Cl)ccc(NS(=O)(=O)c4cc(Cl)cnc4OC)c3F)ccc12] | 2 | [M + H]⁺: 524 |
| 138 | 2-[6-chloro-3-(5-chloro- 2-methylpyridine-3- sulfonamido)-2- fluorophenyl]-N- methylimidazo[1,5- a]pyridazine-5- carboxamide | 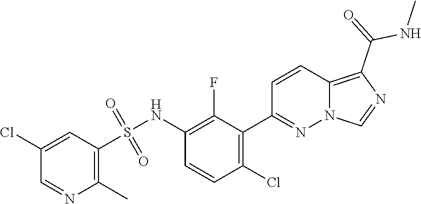 [SMILES: CNC(=O)c1ncn2nc(-c3c(Cl)ccc(NS(=O)(=O)c4cc(Cl)cnc4C)c3F)ccc12] | 9 | [M + H]⁺: 509 |
| 139 | 2-[2-chloro-3-(5-chloro- 2-methylpyridine-3- sulfonamido)-6- fluorophenyl]-N- methylimidazo[1,5- b]pyridazine-5- carboxamide | 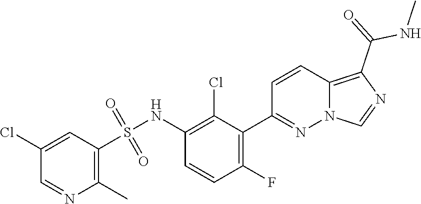 [SMILES: CNC(=O)c1ncn2nc(-c3c(F)ccc(NS(=O)(=O)c4cc(Cl)cnc4C)c3Cl)ccc12] | 9 | [M + H]⁺: 509 |
| 140 | 6-(2-fluoro-5-((5-fluoro- 2-methoxypyridine)-3- sulfonamido)phenyl)-N- methyl-5,6,7,8- tetrahydroimidazo[1,5- a]pyridine-1- carboxamide | 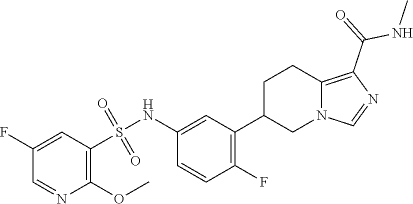 [SMILES: CNC(=O)c1ncn2c1CCC(c1cc(NS(=O)(=O)c3cc(F)cnc3OC)ccc1F)C2] | 1 | [M + H]⁺: 478 |
| 141 | 6-[6-chloro-3-(5-chloro- 2-methoxypyridine-3- sulfonamido)-2- fluorophenyl]-N- methylimidazo[1,5- a]pyrazine-1- carboxamide | 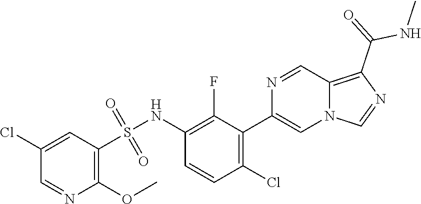 [SMILES: CNC(=O)c1ncn2cc(-c3c(Cl)ccc(NS(=O)(=O)c4cc(Cl)cnc4OC)c3F)ncc12] | 2, 15 | [M + H]⁺: 525 |
| 142 | N-[4-chloro-2-fluoro-3- [1-(1H-imidazol-2- yl)imidazo[1,5- a]pyrazin-6-yl]phenyl]-5- fluoro-2- methoxypyridine-3- sulfonamide | 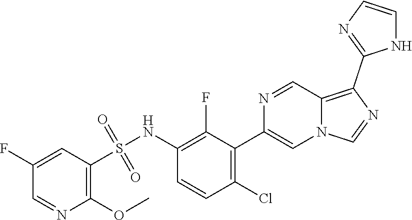 [SMILES: COc1ncc(F)cc1S(=O)(=O)Nc1ccc(Cl)c(-c2cn3cnc(-c4ncc[nH]4)c3cn2)c1F] | 1 | [M + H]⁺: 518 |
| 143 | 5-chloro-N-[3-[1-(4,5- dimethyl-1H-imidazol-2- yl)imidazo[1,5-a]pyridin- 6-yl]-2,4- difluorophenyl]-2- methoxypyridine-3- sulfonamide | 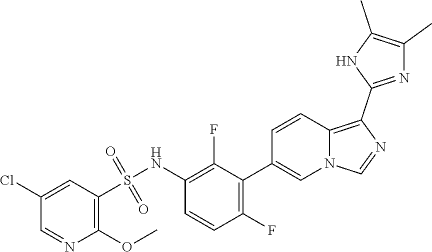 [SMILES: COc1ncc(Cl)cc1S(=O)(=O)Nc1ccc(F)c(-c2ccc3c(-c4nc(C)c(C)[nH]4)ncn3c2)c1F] | 2 | [M + H]⁺: 545 |
| 144 | N-[3-[1-(4,5-dimethyl- 1H-imidazol-2- yl)imidazo[1,5-a]pyridin- 6-yl]-2,4- difluorophenyl]-5-fluoro- 2-methoxypyridine-3- sulfonamide | 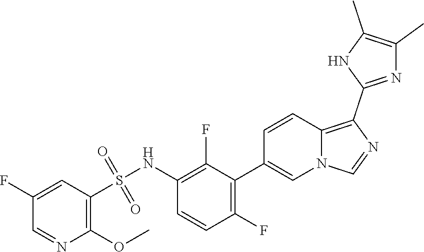 [SMILES: COc1ncc(F)cc1S(=O)(=O)Nc1ccc(F)c(-c2ccc3c(-c4nc(C)c(C)[nH]4)ncn3c2)c1F] | 1 | [M + H]⁺: 529 |
| 145 | N-[3-[1-(4,5-dimethyl- 1H-imidazol-2- yl)imidazo[1,5-a]pyridin- 6-yl]-2,4- difluorophenyl]-5-fluoro- 2-methylpyridine-3- sulfonamide | 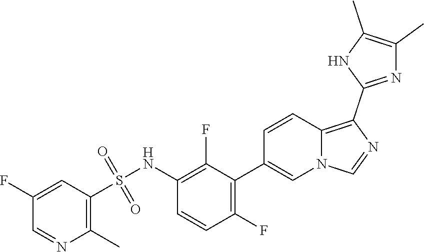 [SMILES: Cc1ncc(F)cc1S(=O)(=O)Nc1ccc(F)c(-c2ccc3c(-c4nc(C)c(C)[nH]4)ncn3c2)c1F] | 4 | [M + H]⁺: 513 |
| 146 | 5-chloro-N-[3-[1-(4,5- dimethyl-1H-imidazol-2- yl)imidazo[1,5-a]pyridin- 6-yl]-2,4- difluorophenyl]-2- methylpyridine-3- sulfonamide | 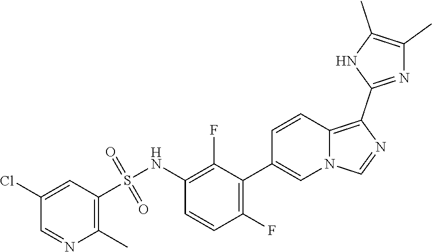 [SMILES: Cc1ncc(Cl)cc1S(=O)(=O)Nc1ccc(F)c(-c2ccc3c(-c4nc(C)c(C)[nH]4)ncn3c2)c1F] | | [M + H]⁺: 529 |
| 147 | 5-chloro-N-(2,4-difluoro- 3-(1-(4,5,6,7-tetrahydro- 1H-benzo[d]imidazol-2- yl)imidazo[1,5- a]pyridin-6-yl)phenyl)-2- methoxypyridine-3- sulfonamide | 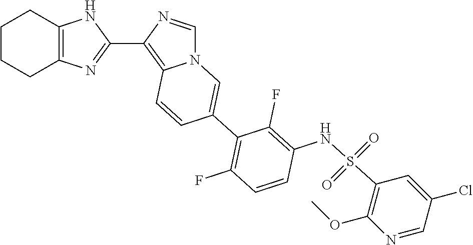 [SMILES: COc1ncc(Cl)cc1S(=O)(=O)Nc1ccc(F)c(-c2ccc3c(-c4nc5c([nH]4)CCCC5)ncn3c2)c1F] | 2 | [M + H]⁺: 571 |
| 148 | N-[2,4-difluoro-3-[1- (4,5,6,7-tetrahydro-1H- 1,3-benzodiazol-2- yl)imidazo[1,5-a]pyridin- 6-yl]phenyl]-5-fluoro-2- methoxypyridine-3- sulfonamide | 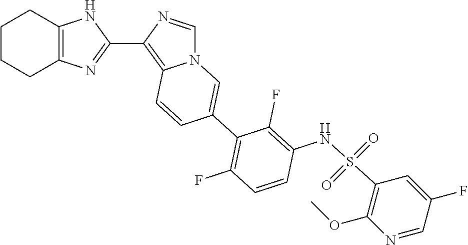 [SMILES: COc1ncc(F)cc1S(=O)(=O)Nc1ccc(F)c(-c2ccc3c(-c4nc5c([nH]4)CCCC5)ncn3c2)c1F] | 1 | [M + H]⁺: 555 |
| 149 | N-[2,4-difluoro-3-[1- (4,5,6,7-tetrahydro-1H- 1,3-benzodiazol-2- yl)imidazo[1,5-a]pyridin- 6-yl]phenyl]-5-fluoro-2- methylpyridine-3- sulfonamide | 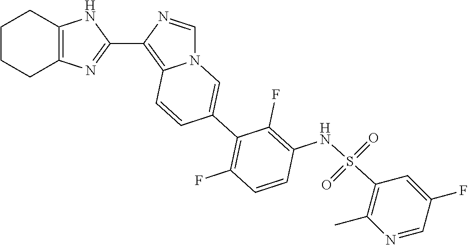 [SMILES: Cc1ncc(F)cc1S(=O)(=O)Nc1ccc(F)c(-c2ccc3c(-c4nc5c([nH]4)CCCC5)ncn3c2)c1F] | 4 | [M + H]⁺: 539 |
| 150 | 5-chloro-N-[2,4-difluoro- 3-[1-(4,5,6,7-tetrahydro- 1H-1,3-benzodiazol-2- yl)imidazo[1,5-a]pyridin- 6-yl]phenyl]-2- methylpyridine-3- sulfonamide | 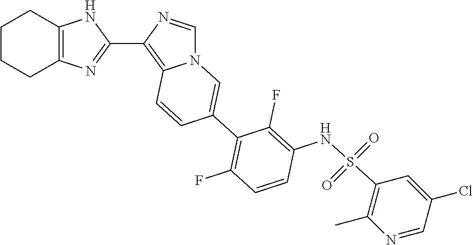 [SMILES: Cc1ncc(Cl)cc1S(=O)(=O)Nc1ccc(F)c(-c2ccc3c(-c4nc5c([nH]4)CCCC5)ncn3c2)c1F] | | [M + H]⁺: 555 |
| 151 | N-[2-chloro-4-fluoro-3- [1-(1H-imidazol-2- yl)imidazo[1,5- a]pyrazin-6-yl]phenyl]-5- fluoro-2- methoxypyridine-3- sulfonamide | 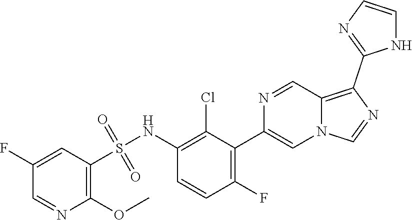 [SMILES: COc1ncc(F)cc1S(=O)(=O)Nc1ccc(F)c(-c2cn3cnc(-c4ncc[nH]4)c3cn2)c1Cl] | 1 | [M + H]⁺: 518 |
| 152-1 | (6R)-6-[2-chloro-6- fluoro-3-(5-fluoro-2- methoxypyridine-3- sulfonamido)phenyl]-N- methyl-5H,6H,7H,8H- imidazo[1,5-a]pyridine- 1-carboxamide | 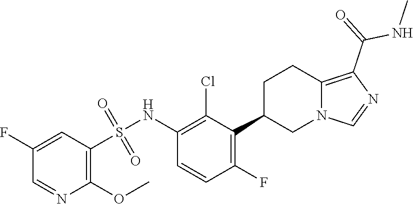 [SMILES: CNC(=O)c1ncn2c1CC[C@H](c1c(F)ccc(NS(=O)(=O)c3cc(F)cnc3OC)c1Cl)C2] | 1 | [M + H]⁺: 512 |
| 152-2 | (6S)-6-[2-chloro-6- fluoro-3-(5-fluoro-2- methoxypyridine-3- sulfonamido)phenyl]-N- methyl-5H,6H,7H,8H- imidazo[1,5-a]pyridine- 1-carboxamide | 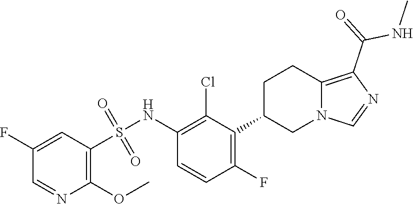 [SMILES: CNC(=O)c1ncn2c1CC[C@@H](c1c(F)ccc(NS(=O)(=O)c3cc(F)cnc3OC)c1Cl)C2] | 1 | [M + H]⁺: 512 |
| 153 | methyl 2-[6-[2,6- difluoro-3-(5-fluoro-2- methoxypyridine-3- sulfonamido)phenyl] imidazo[1,5-a]pyrazin- 1-yl]-3H-1,3-benzo- diazole-5-carboxylate | 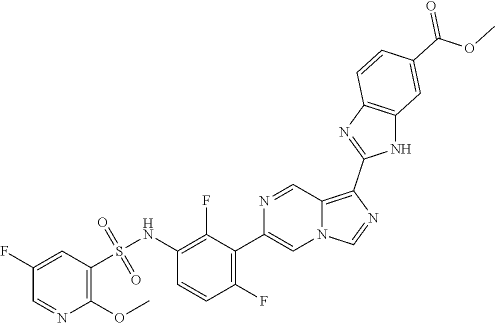 [SMILES: COC(=O)c1ccc2nc(-c3ncn4cc(-c5c(F)ccc(NS(=O)(=O)c6cc(F)cnc6OC)c5F)ncc34)[nH]c2c1] | 1, 11 | [M + H]⁺: 610 |
| 154-1 | (6S)-6-[3-(5-chloro-2- methoxypyridine-3- sulfonamido)-2,6- difluorophenyl]-N- methyl-5H,6H,8H- imidazo[4,3- c][1,4]oxazine-1- carboxamide | 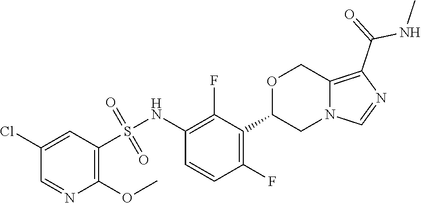 [SMILES: CNC(=O)c1ncn2c1CO[C@@H](c1c(F)ccc(NS(=O)(=O)c3cc(Cl)cnc3OC)c1F)C2] | 2 | [M + H]⁺: 514 |
| 154-2 | (6R)-6-[3-(5-chloro-2- methoxypyridine-3- sulfonamido)-2,6- difluorophenyl]-N- methyl-5H,6H,8H- imidazo[4,3- c][1,4]oxazine-1- carboxamide | 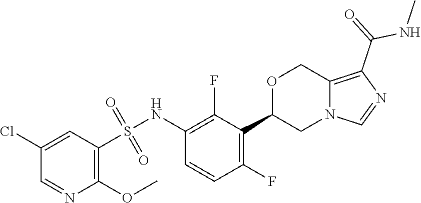 [SMILES: CNC(=O)c1ncn2c1CO[C@H](c1c(F)ccc(NS(=O)(=O)c3cc(Cl)cnc3OC)c1F)C2] | 2 | [M + H]⁺: 514 |
| 155-1 | (6S)-6-[6-chloro-2- fluoro-3-(5-fluoro-2- methoxypyridine-3- sulfonamido)phenyl]-N- methyl-5H,6H,7H,8H- imidazo[1,5-a]pyridine- 1-carboxamide | 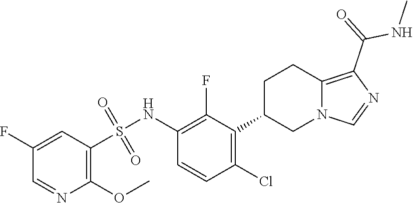 [SMILES: CNC(=O)c1ncn2c1CC[C@@H](c1c(Cl)ccc(NS(=O)(=O)c3cc(F)cnc3OC)c1F)C2] | 1, 14-a | [M + H]⁺: 512 |
| 155-2 | (6R)-6-[6-chloro-2- fluoro-3-(5-fluoro-2- methoxypyridine-3- sulfonamido)phenyl]-N- methyl-5H,6H,7H,8H- imidazo[1,5-a]pyridine- 1-carboxamide | 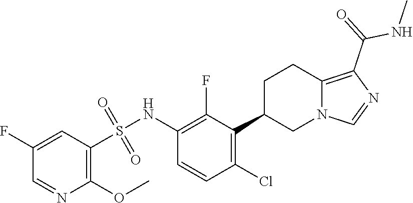 [SMILES: CNC(=O)c1ncn2c1CC[C@H](c1c(Cl)ccc(NS(=O)(=O)c3cc(F)cnc3OC)c1F)C2] | 1, 14-a | [M + H]⁺: 512 |
| 156 | N-[2,4-difluoro-3-[1-(4- phenyl-1H-imidazol-2- yl)imidazo[1,5- a]pyrazin-6-yl]phenyl]-5- fluoro-2- methoxypyridine-3- sulfonamide | 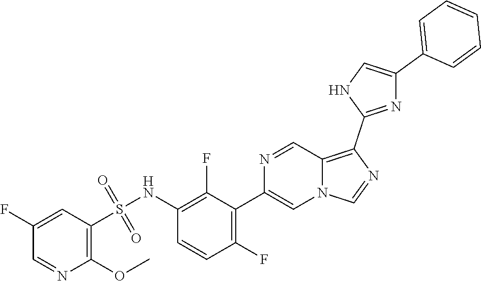 [SMILES: COc1ncc(F)cc1S(=O)(=O)Nc1ccc(F)c(-c2cn3cnc(-c4nc(-c5ccccc5)c[nH]4)c3cn2)c1F] | 1, 12 | [M + H]⁺: 578 |
| 157 | 6-[3-(5-chloro-2- methoxypyridine-3- sulfonamido)-2,6- difluorophenyl]-N- isopropylimidazo[1,5- a]pyrazine-1- carboxamide | 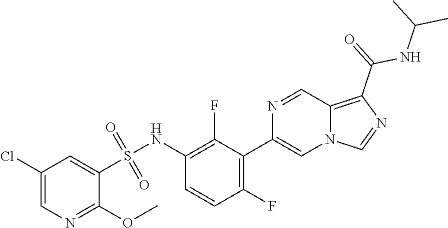 [SMILES: COc1ncc(Cl)cc1S(=O)(=O)Nc1ccc(F)c(-c2cn3cnc(C(=O)NC(C)C)c3cn2)c1F] | | [M + H]⁺: 537 |
| 158 | 6-[3-(5-chloro-2- methoxypyridine-3- sulfonamido)-2,6- difluorophenyl]-N-(2- methylpropyl)imidazo [1,5-a]pyrazine-1- carboxamide | 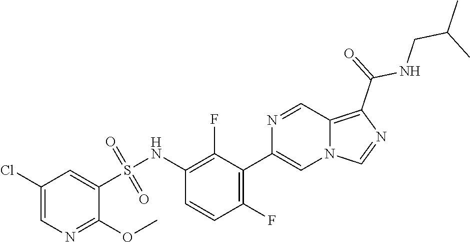 [SMILES: COc1ncc(Cl)cc1S(=O)(=O)Nc1ccc(F)c(-c2cn3cnc(C(=O)NCC(C)C)c3cn2)c1F] | | [M + H]⁺: 551 |
| 159 | 6-[3-(5-chloro-2- methoxypyridine-3- sulfonamido)-2,6- difluorophenyl]-N- (2,2,2-trifluoroethyl) imidazo[1,5-a] pyrazine-1- carboxamide | 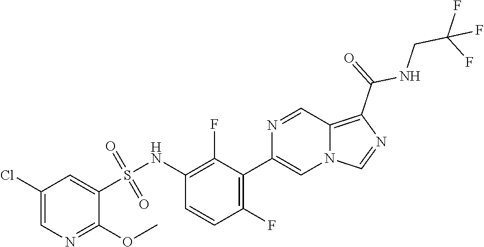 [SMILES: COc1ncc(Cl)cc1S(=O)(=O)Nc1ccc(F)c(-c2cn3cnc(C(=O)NCC(F)(F)F)c3cn2)c1F] | | [M + H]⁺: 577 |
| 160 | 6-[3-(5-chloro-2- methoxypyridine-3- sulfonamido)-2,6- difluorophenyl]-5- fluoro-N-methylimidazo [1,5-a]pyrazine-1- carboxamide | 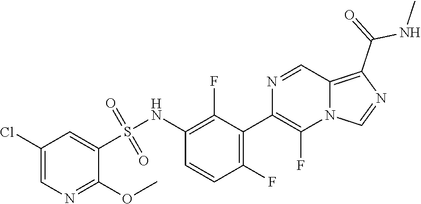 [SMILES: CNC(=O)c1ncn2c(F)c(-c3c(F)ccc(NS(=O)(=O)c4cc(Cl)cnc4OC)c3F)ncc12] | 2 | [M + H]⁺: 527 |
| 161 | 6-[3-(5-chloro-2- methoxypyridine-3- sulfonamido)-2,6- difluorophenyl]-N-(2- hydroxyethyl)imidazo [1,5-a]pyrazine-1- carboxamide | 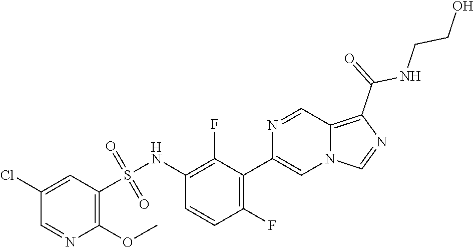 [SMILES: COc1ncc(Cl)cc1S(=O)(=O)Nc1ccc(F)c(-c2cn3cnc(C(=O)NCCO)c3cn2)c1F] | | [M + H]⁺: 539 |
| 162 | 6-[3-(5-chloro-2- methoxypyridine-3- sulfonamido)-2,6- difluorophenyl]-N-(1- methylpyrazol-4- yl)imidazo[1,5-a] pyrazine-1- carboxamide | 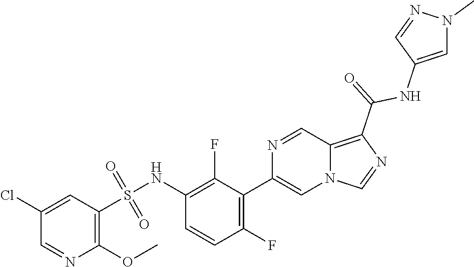 [SMILES: COc1ncc(Cl)cc1S(=O)(=O)Nc1ccc(F)c(-c2cn3cnc(C(=O)Nc4cnn(C)c4)c3cn2)c1F] | | [M + H]⁺: 575 |
| 163 | 6-[3-(5-chloro-2- methoxypyridine-3- sulfonamido)-2,6- difluorophenyl]-N- (oxan-4-ylmethyl) imidazo[1,5-a] pyrazine-1-carboxamide | 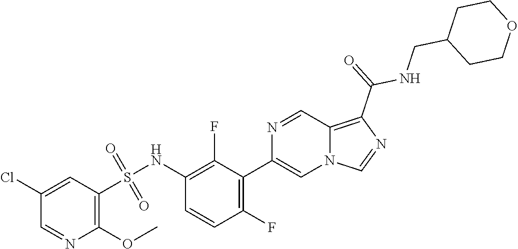 [SMILES: COc1ncc(Cl)cc1S(=O)(=O)Nc1ccc(F)c(-c2cn3cnc(C(=O)NCC4CCOCC4)c3cn2)c1F] | | [M + H]⁺: 593 |
| 164 | 6-[3-(5-chloro-2- methoxypyridine-3- sulfonamido)-2,6- difluorophenyl]-N-(2- methanesulfonylethyl) imidazo[1,5-a] pyrazine-1-carboxamide | 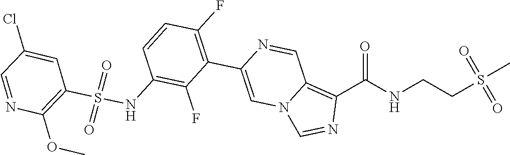 [SMILES: COc1ncc(Cl)cc1S(=O)(=O)Nc1ccc(F)c(-c2cn3cnc(C(=O)NCCS(C)(=O)=O)c3cn2)c1F] | | [M + H]⁺: 601 |
| 165 | 6-[3-(5-chloro-2- methoxypyridine-3- sulfonamido)-2,6- difluorophenyl]-N- ethylimidazo[1,5-a] pyrazine-1- carboxamide | 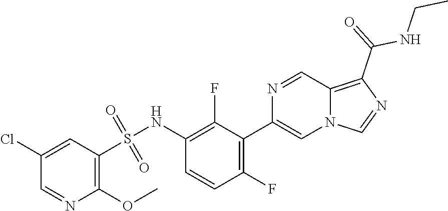 [SMILES: CCNC(=O)c1ncn2cc(-c3c(F)ccc(NS(=O)(=O)c4cc(Cl)cnc4OC)c3F)ncc12] | | [M + H]⁺: 523 |
| 166 | 6-[3-(5-chloro-2- methoxypyridine-3- sulfonamido)-2,6- difluorophenyl]-N- (cyclopropylmethyl) imidazo[1,5-a] pyrazine-1- carboxamide | 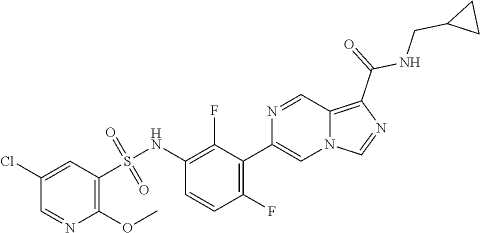 [SMILES: COc1ncc(Cl)cc1S(=O)(=O)Nc1ccc(F)c(-c2cn3cnc(C(=O)NCC4CC4)c3cn2)c1F] | | [M + H]⁺: 549 |
| 167 | 6-[3-(5-chloro-2- ethoxypyridine-3- sulfonamido)-2,6- difluorophenyl]-N- methylimidazo[1,5- a]pyrazine-1- carboxamide | 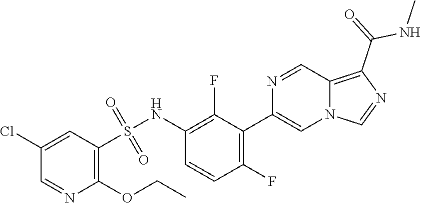 [SMILES: CCOc1ncc(Cl)cc1S(=O)(=O)Nc1ccc(F)c(-c2cn3cnc(C(=O)NC)c3cn2)c1F] | | [M + H]⁺: 523 |
| 168 | 6-[3-(5-chloro-2- isopropoxypyridine-3- sulfonamido)-2,6- difluorophenyl]-N- methylimidazo[1,5- a]pyrazine-1- carboxamide | 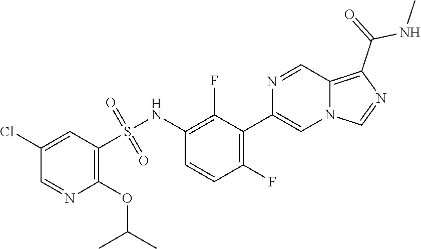 [SMILES: CNC(=O)c1ncn2cc(-c3c(F)ccc(NS(=O)(=O)c4cc(Cl)cnc4OC(C)C)c3F)ncc12] | | [M + H]⁺: 537 |
| 169 | 6-[3-(5-chloro-2- cyclopropoxypyridine-3- sulfonamido)-2,6- difluorophenyl]-N- methylimidazo[1,5- a]pyrazine-1- carboxamide | 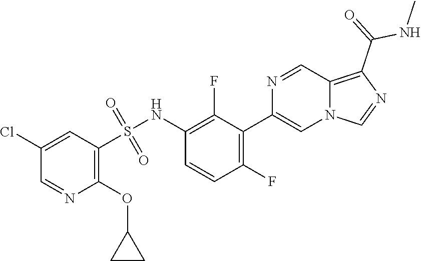 [SMILES: CNC(=O)c1ncn2cc(-c3c(F)ccc(NS(=O)(=O)c4cc(Cl)cnc4OC4CC4)c3F)ncc12] | | [M + H]⁺: 535 |
| 170 | 6-[2,6-difluoro-3-(5- fluoro-2- methoxypyridine-3- sulfonamido)phenyl]-5- fluoro-N- methylimidazo[1,5- a]pyrazine-1- carboxamide | 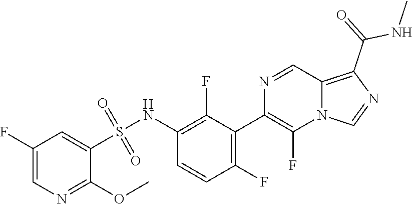 [SMILES: CNC(=O)c1ncn2c(F)c(-c3c(F)ccc(NS(=O)(=O)c4cc(F)cnc4OC)c3F)ncc12] | 1 | [M + H]⁺: 511 |
| 171 | 6-[3-(5-chloro-2- methoxypyridine-3- sulfonamido)-2,6- difluorophenyl] imidazo[1,5-a] pyrazine-1- carboxamide | 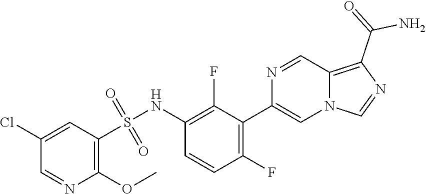 [SMILES: COc1ncc(Cl)cc1S(=O)(=O)Nc1ccc(F)c(-c2cn3cnc(C(N)=O)c3cn2)c1F] | | [M + H]⁺: 495 |
| 172 | 6-[3-(5-chloro-2- methoxypyridine-3- sulfonamido)-2,6- difluorophenyl]-N-[3- (dimethylamino)propyl] imidazo[1,5-a]pyrazine- 1-carboxamide | 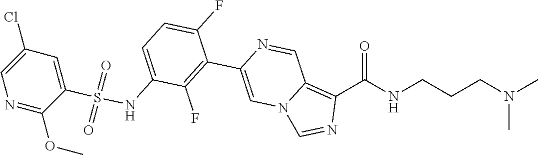 [SMILES: COc1ncc(Cl)cc1S(=O)(=O)Nc1ccc(F)c(-c2cn3cnc(C(=O)NCCCN(C)C)c3cn2)c1F] | | [M + H]⁺: 580 |
| 173-1 | (R)-6-(3-((5-chloro-2- methoxypyridine)-3- sulfonamido)-2,6- difluorophenyl)-N-(1- methylpyrrolidin-3- yl)imidazo[1,5- a]pyrazine-1- carboxamide | 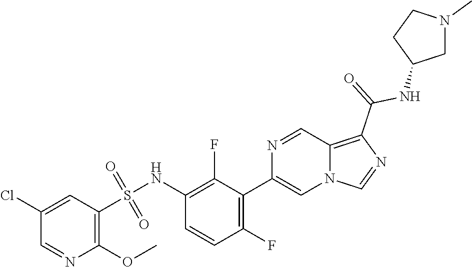 [SMILES: COc1ncc(Cl)cc1S(=O)(=O)Nc1ccc(F)c(-c2cn3cnc(C(=O)N[C@@H]4CCN(C)C4)c3cn2)c1F] | | [M + H]⁺: 578 |
| 173-2 | (S)-6-(3-((5-chloro-2- methoxypyridine)-3- sulfonamido)-2,6- difluorophenyl)-N-(1- methylpyrrolidin-3- yl)imidazo[1,5- a]pyrazine-1- carboxamide | 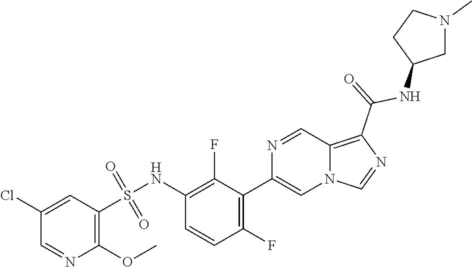 [SMILES: COc1ncc(Cl)cc1S(=O)(=O)Nc1ccc(F)c(-c2cn3cnc(C(=O)N[C@H]4CCN(C)C4)c3cn2)c1F] | | [M + H]⁺: 578 |
| 174 | 6-[3-(5-chloro-2- methoxypyridine-3- sulfonamido)-2,6- difluorophenyl]-N-(1,2- oxazol-4-yl)imidazo [1,5-a]pyrazine-1- carboxamide | 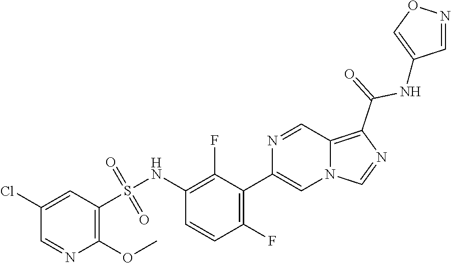 [SMILES: COc1ncc(Cl)cc1S(=O)(=O)Nc1ccc(F)c(-c2cn3cnc(C(=O)Nc4cnoc4)c3cn2)c1F] | | [M + H]⁺: 562 |
| 175 | 6-[3-(5-chloro-2- methoxypyridine-3- sulfonamido)-2,6- difluorophenyl]-N-[2- (morpholin-4- yl)ethyl]imidazo[1,5- a]pyrazine-1- carboxamide | 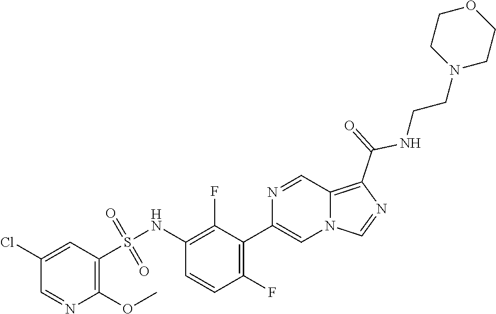 [SMILES: COc1ncc(Cl)cc1S(=O)(=O)Nc1ccc(F)c(-c2cn3cnc(C(=O)NCCN4CCOCC4)c3cn2)c1F] | | [M + H]⁺: 608 |
| 176 | 6-[3-[5-chloro-2-(2- methylpropoxy)pyridine- 3-sulfonamido]-2,6- difluorophenyl]-N- methylimidazo[1,5- a]pyrazine-1- carboxamide | 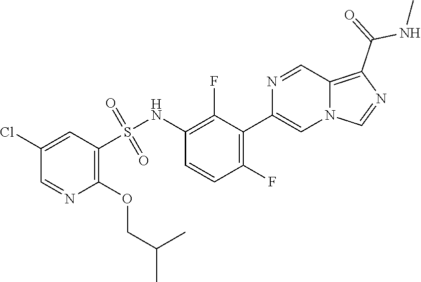 [SMILES: CNC(=O)c1ncn2cc(-c3c(F)ccc(NS(=O)(=O)c4cc(Cl)cnc4OCC(C)C)c3F)ncc12] | | [M + H]⁺: 551 |
| 177 | N-[3-[1-(5-cyclopropyl- 3H-imidazol-4- yl)imidazo[1,5-a] pyrazin-6-yl]-2,4- difluorophenyl]-5- fluoro-2-methoxy- pyridine-3-sulfonamide | 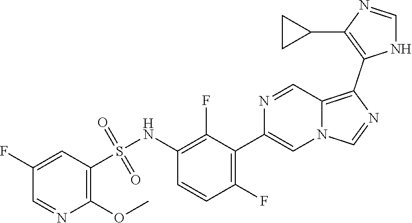 [SMILES: COc1ncc(F)cc1S(=O)(=O)Nc1ccc(F)c(-c2cn3cnc(-c4[nH]cnc4C4CC4)c3cn2)c1F] | 1, 12 | [M + H]⁺: 542 |
| 178 | N-[3-[1-(4-benzyl-1H- imidazol-2- yl)imidazo[1,5-a] pyrazin-6-yl]-2,4- difluorophenyl]-5- fluoro-2-methoxy- pyridine-3-sulfonamide | 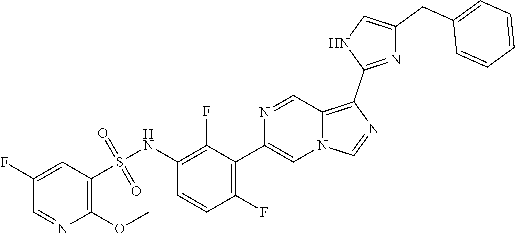 [SMILES: COc1ncc(F)cc1S(=O)(=O)Nc1ccc(F)c(-c2cn3cnc(-c4nc(Cc5ccccc5)c[nH]4)c3cn2)c1F] | 12, 1 | [M + H]⁺: 592 |
| 179 | 6-[3-(5-chloro-2- methoxypyridine-3- sulfonamido)-2,6- difluorophenyl]-N-[2- (dimethylamino)ethyl] imidazo[1,5-a] pyrazine-1- carboxamide | 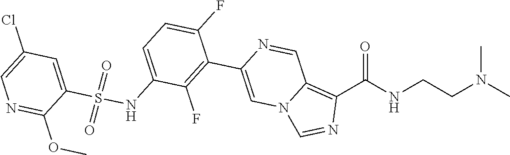 [SMILES: COc1ncc(Cl)cc1S(=O)(=O)Nc1ccc(F)c(-c2cn3cnc(C(=O)NCCN(C)C)c3cn2)c1F] | | [M + H]⁺: 566 |
| 180 | 6-[3-(5-chloro-2- methoxypyridine-3- sulfonamido)-2,6- difluorophenyl]-N-[2-(2- oxopyrrolidin-1- yl)ethyl]imidazo[1,5-a] pyrazine-1- carboxamide | 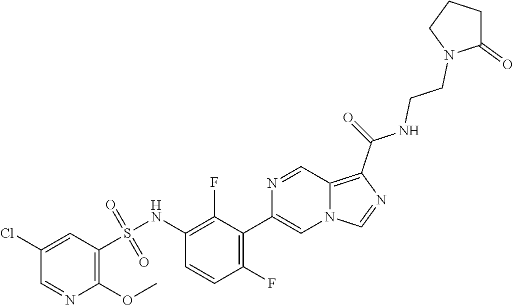 [SMILES: COc1ncc(Cl)cc1S(=O)(=O)Nc1ccc(F)c(-c2cn3cnc(C(=O)NCCN4CCCC4=O)c3cn2)c1F] | | [M + H]⁺: 606 |
| 181 | 6-[3-(5-chloro-2- methoxypyridine-3- sulfonamido)-2,6- difluorophenyl]-N-[4- [(dimethylamino)methyl] phenyl]imidazo[1,5-a] pyrazine-1-carboxamide | 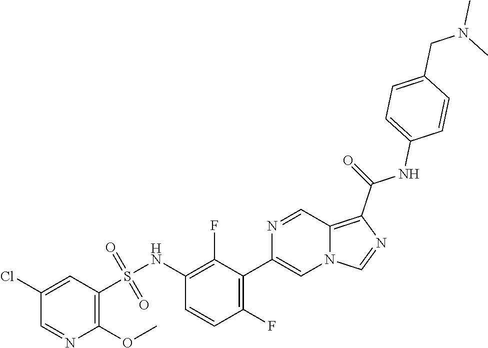 [SMILES: COc1ncc(Cl)cc1S(=O)(=O)Nc1ccc(F)c(-c2cn3cnc(C(=O)Nc4ccc(CN(C)C)cc4)c3cn2)c1F] | | [M + H]⁺: 628 |
| 182 | 6-[3-[5-chloro-2-(2,2,2- trifluoroethoxy)pyridine- 3-sulfonamido]-2,6- difluorophenyl]-N- methylimidazo[1,5-a] pyrazine-1- carboxamide | 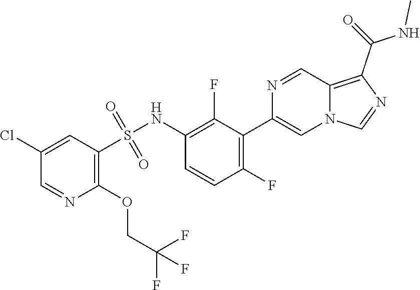 [SMILES: CNC(=O)c1ncn2cc(-c3c(F)ccc(NS(=O)(=O)c4cc(Cl)cnc4OCC(F)(F)F)c3F)ncc12] | | [M + H]⁺: 577 |
| 183 | 6-[3-[2-(benzyloxy)-5- chloropyridine-3- sulfonamido]-2,6- difluorophenyl]-N- methylimidazo[1,5- a]pyrazine-1- carboxamide | 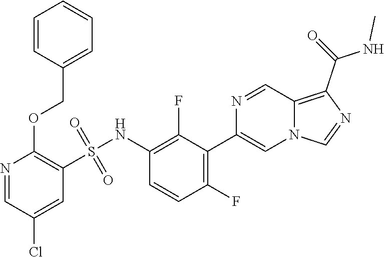 [SMILES: CNC(=O)c1ncn2cc(-c3c(F)ccc(NS(=O)(=O)c4cc(Cl)cnc4OCc4ccccc4)c3F)ncc12] | | [M + H]⁺: 585 |
| 184 | 6-[2,6-difluoro-3-(5- fluoro-2-methylpyridine- 3-sulfonamido)phenyl]- 5-fluoro-N- methylimidazo[1,5-a] pyrazine-1-carboxamide | 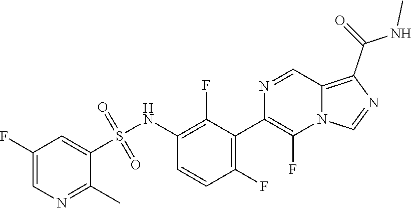 [SMILES: CNC(=O)c1ncn2c(F)c(-c3c(F)ccc(NS(=O)(=O)c4cc(F)cnc4C)c3F)ncc12] | 4 | [M + H]⁺: 495 |
| 185 | N-(2,4-difluoro-3-[1-[5- (4-methylpiperazin-1-yl)- 3H-1,3-benzodiazol-2- yl]imidazo[1,5-a]pyridin- 6-yl]phenyl)-5-fluoro-2- methoxypyridine-3- sulfonamide | 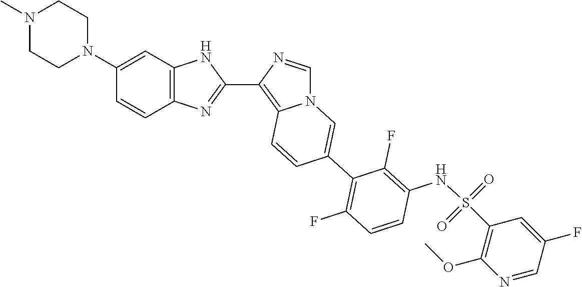 [SMILES: COc1ncc(F)cc1S(=O)(=O)Nc1ccc(F)c(-c2ccc3c(-c4nc5ccc(N6CCN(C)CC6)cc5[nH]4)ncn3c2)c1F] | 1 | [M + H]⁺: 649 |
| 186 | 6-[3-(5-chloro-2- methylpyridine-3- sulfonamido)-2,6- difluorophenyl]-5- fluoro-N-methylimidazo- [1,5-a]pyrazine-1- carboxamide | 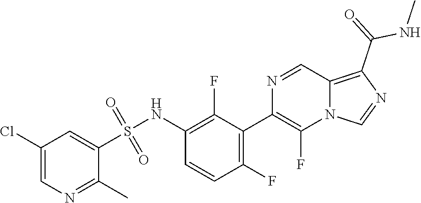 [SMILES: CNC(=O)c1ncn2c(F)c(-c3c(F)ccc(NS(=O)(=O)c4cc(Cl)cnc4C)c3F)ncc12] | | [M + H]⁺: 511 |

### Example 202—Biochemical Assay for Modulation (Inhibition/Activation) of GCN2

Exemplary compounds from the above Examples were tested for ability to inhibit GCN2 activity using a time resolved fluorescence energy transfer (TR-FRET) assay. Assay procedures and results are described below for Examples 16-201.

### Part I—Procedures for TR-FRET Assay

GCN2 protein was obtained from Carna Biosciences (cat #05-153). The protein was diluted in assay buffer (ThermoFisher Scientific, #PV6135), 2 mM dithiothreitol (DTT), to obtain a final concentration of 2 nM and 5 μL was plated in a 384-well white assay plate. Test compounds were serially diluted to 11 concentrations by 3-fold dilution in DMSO and 10 nL of stock was plated into 384 well white assay plate. DMSO was used as a vehicle control. GFP-eIF2a protein was obtained from ThermoFisher (cat #PV4809). The protein was diluted in assay buffer to a 2× concentration of 200 nM along with 300 μM ATP (final concentration of 100 nM GFP-eIF2a and 150 μM ATP) in the presence of 2 mM DTT and a 5 μL aliquot was added to each well containing the GCN2 protein and test compound. The plate was incubated in the dark at 25° C. for 1.5 hours, shaking at 1250 rpm. Tb-anti P-eIF2a (ThermoFisher cat #PV4815) was diluted to a to a concentration of 1 nM in TR-FRET Dilution Buffer (ThermoFisher cat #PV3574). 10 μL of the Tb-anti P-eIF2a solution was added to the TR-FRET reaction. The plate was incubated in the dark for 2 h at 25° C. shaking at 600 rpm. The FRET signal from the plate was read on a Envision (PerkinElmer) plate reader:

Label 1: Excitation: 340 nm, bandwidth 30 nm; Emission: 495 nm, bandwidth 10 nm. Lag time: 100 μsec. Integration time: 400 μsec. Flashes: 30.

Label 2: Excitation: 340 nm, bandwidth 30 nm; Emission: 520 nm, bandwidth 25 nm. Lag time: 100 μsec. Integration time: 400 μsec. Flashes: 30

The data were analyzed using GraphPad Prism employing a 4-parameter sigmoidal curve fit.

### Part II—Results

Experimental results are provided in Table 2 below. The symbol “++++” indicates an IC₅₀ less than 0.0500 μM. The symbol “+++” indicates an IC₅₀ in the range of 0.0500 μM to 0.5000 μM. The symbol “++” indicates an IC₅₀ in the range of greater than 0.5000 μM to 1.0000 μM. The symbol “+” indicates an IC₅₀ greater than 1.0000 μM. The symbol “N/A” indicates that no data was available.

TABLE 2

| Compound No. | IC₅₀/μM |
|---|---|
| 1 | ++++ |
| 2 | ++++ |
| 3 | ++++ |
| 4 | ++++ |
| 5 | ++++ |
| 6 | ++++ |
| 7 | ++++ |
| 8 | +++ |
| 9 | ++++ |
| 10 | ++++ |
| 11 | +++ |
| 12 | ++++ |
| 13 | ++++ |
| 14 | ++++ |
| 15 | + |
| 16 | +++ |
| 17 | +++ |
| 18 | ++++ |
| 19 | ++++ |
| 20 | ++++ |
| 21 | ++++ |
| 22 | +++ |
| 23 | +++ |
| 24 | + |
| 25 | ++++ |
| 26 | ++++ |
| 27-1 | +++ |
| 27-2 | +++ |
| 28 | ++++ |
| 29 | ++++ |
| 30 | ++++ |
| 31 | ++++ |
| 32 | ++++ |
| 33-1 | + |
| 33-2 | ++ |
| 34 | ++++ |
| 35 | ++++ |
| 36 | ++++ |
| 37 | ++++ |
| 38 | ++++ |
| 39 | ++++ |
| 40 | ++++ |
| 41 | ++++ |
| 42 | ++++ |
| 43 | ++++ |
| 44 | ++++ |
| 45 | ++++ |
| 46 | ++++ |
| 47 | ++++ |
| 48 | ++++ |
| 49 | ++++ |
| 50 | ++++ |
| 51 | ++++ |
| 52 | ++++ |
| 53 | +++ |
| 54 | +++ |
| 55-1 | ++++ |
| 55-2 | ++ |
| 56 | ++++ |
| 57 | ++++ |
| 58 | ++++ |
| 59 | ++++ |
| 60 | ++++ |
| 61 | ++++ |
| 62 | ++++ |
| 63 | ++++ |
| 64-1 | ++++ |
| 64-2 | ++++ |
| 65 | ++++ |
| 66 | ++++ |
| 67-1 | ++++ |
| 67-2 | +++ |
| 68-1 | +++ |
| 68-2 | +++ |
| 69 | ++++ |
| 70 | ++++ |
| 71 | ++++ |
| 72 | ++++ |
| 73-1 | +++ |
| 73-2 | +++ |
| 74 | ++++ |
| 75 | ++++ |
| 76 | ++++ |
| 77 | ++++ |
| 78 | ++++ |
| 79-1 | ++++ |
| 79-2 | ++++ |
| 80-1 | ++++ |
| 80-2 | ++++ |
| 81-1 | ++++ |
| 81-2 | ++++ |
| 82-1 | +++ |
| 82-2 | +++ |
| 83 | ++++ |
| 84-1 | ++++ |
| 84-2 | ++++ |
| 85 | ++++ |
| 86 | ++++ |
| 87 | ++++ |
| 88 | ++++ |
| 89 | ++++ |
| 90 | +++ |
| 91 | +++ |
| 92 | +++ |
| 93 | ++++ |
| 94 | ++ |
| 95 | ++++ |
| 96-1 | +++ |
| 96-2 | ++ |
| 97 | ++++ |
| 98 | ++++ |
| 99 | ++++ |
| 100 | ++ |
| 101 | ++++ |
| 102 | +++ |
| 103 | ++++ |
| 104 | ++++ |
| 105 | ++++ |
| 106 | ++++ |
| 107 | ++++ |
| 108 | ++++ |
| 109 | ++++ |
| 110 | ++++ |
| 110 | ++++ |
| 111 | ++++ |
| 112 | ++++ |
| 113 | ++++ |
| 114 | ++++ |
| 115 | ++++ |
| 116 | ++++ |
| 117 | +++ |
| 118 | ++++ |
| 119 | ++++ |
| 120 | ++ |
| 121 | ++++ |
| 122 | +++ |
| 123 | ++++ |
| 124 | ++++ |
| 125 | ++++ |
| 126 | ++++ |
| 127 | +++ |
| 128 | +++ |
| 129 | ++++ |
| 130-1 | ++++ |
| 130-2 | ++++ |
| 131-1 | ++++ |
| 131-2 | ++++ |
| 132 | + |
| 133 | ++++ |
| 134-1 | ++++ |
| 134-2 | ++++ |
| 135-1 | +++ |
| 135-2 | +++ |
| 136-1 | ++++ |
| 136-2 | ++++ |
| 137 | ++++ |
| 138 | ++++ |
| 139 | ++++ |
| 140 | + |
| 140 | ++++ |
| 141 | ++++ |
| 142 | ++++ |
| 143 | ++++ |
| 144 | ++++ |
| 145 | ++++ |
| 146 | ++++ |
| 147 | ++++ |
| 148 | ++++ |
| 149 | ++++ |
| 150 | ++++ |
| 151 | ++++ |
| 152-1 | +++ |
| 152-2 | +++ |
| 153 | ++++ |
| 154-1 | +++ |
| 154-2 | +++ |
| 155-1 | ++++ |
| 155-2 | +++ |
| 156 | ++++ |
| 157 | ++++ |
| 158 | ++++ |
| 159 | ++++ |
| 160 | ++++ |
| 161 | ++++ |
| 162 | ++++ |
| 163 | ++++ |
| 164 | ++++ |
| 165 | ++++ |
| 166 | ++++ |
| 167 | +++ |
| 168 | +++ |
| 169 | +++ |
| 170 | +++ |
| 171 | ++++ |
| 172 | +++ |
| 173-1 | +++ |
| 173-2 | ++++ |
| 174 | ++++ |
| 175 | ++++ |
| 176 | ++ |
| 177 | +++ |
| 178 | ++++ |
| 179 | +++ |
| 180 | +++ |
| 181 | ++++ |
| 182 | ++ |
| 183 | + |
| 184 | +++ |
| 185 | ++++ |
| 186 | ++++ |

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

1. (canceled)

2. A compound represented by Formula (Ia): 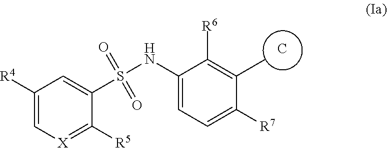
or a pharmaceutically acceptable salt thereof, wherein:
C is selected from 9-membered bicyclic heterocyclyl and 8-9 membered bicyclic heteroaryl selected from the group consisting of 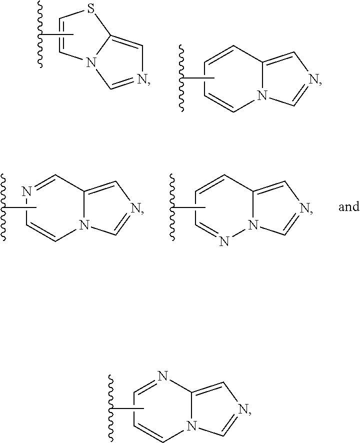
wherein the 9-membered bicyclic heterocyclyl or 8-9 membered bicyclic heteroaryl may be optionally substituted on one or more available carbons by one, two, three, or more substituents each independently selected from R³; wherein the 9-membered bicyclic heterocyclyl is partially unsaturated and contains at least two nitrogen atoms; wherein, if the 9-membered bicyclic heterocyclyl or 8-9 membered bicyclic heteroaryl contains a substitutable ring nitrogen atom, that ring nitrogen atom may be optionally substituted by C₁₋₆alkyl;
X is selected from the group consisting of CH, C(R⁸), and N;
R³ is independently, for each occurrence, selected from the group consisting of halogen, C₁₋₆alkyl, C₃₋₆cycloalkyl, cyano, C₁₋₆alkoxyl, hydroxyl, oxo, phenyl, —C(O)N(R^{A})(R^{B}), —N(R^{A})(R^{B}), 5-10 membered heterocyclyl, and 5-10 membered heteroaryl, wherein the C₁₋₆alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl;
R⁴ is selected from the group consisting of halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{4a};
R⁵ is selected from the group consisting of halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{5a};
R⁶ is selected from the group consisting of halogen, C₁₋₆alkyl, cyano, C₁₋₆alkoxyl, and C₃₋₆cycloalkyl, wherein the C₁₋₆alkyl and C₁₋₆cycloalkyl may be optionally substituted on one or more available carbons by one, two, three, or more substituents each independently selected from R^{6a};
R⁷ is selected from the group consisting of fluoro, chloro, methyl, and cyano;
R⁸ is selected from the group consisting of halogen, C₁₋₆alkyl, C₃₋₆cycloalkyl, cyano, hydroxyl, oxo, C₁₋₆alkoxyl, —O—C₃₋₆cycloalkyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from R^{1a};
or when X is C(R⁸), R⁸ and R⁵ may optionally combine together with the atoms to which they are attached to form a 3-7 membered carbocyclyl, 3-7 membered heterocyclyl, or 5-10 membered heteroaryl, wherein the 3-7 membered carbocyclyl, 3-7 membered heterocyclyl, or 5-10 membered heteroaryl may be optionally substituted on one or more available carbons by one, two, three, or more hydroxyl substituents; wherein, if the 3-7 membered carbocyclyl, 3-7 membered heterocyclyl, or 5-10 membered heteroaryl contains a substitutable ring nitrogen atom, that ring nitrogen atom may be optionally substituted by C₁₋₆alkyl;
R^{A} is hydrogen or C₁₋₆alkyl;
R^{B} is selected from the group consisting of hydrogen, C₁₋₆alkyl, C₃₋₆cycloalkyl, phenyl, 5-6 membered heterocyclyl, and 5-6 membered heteroaryl, wherein the C₁₋₆alkyl or phenyl may be optionally substituted on one or more available carbons by one, two, three, or more substituents each independently selected from R^{e}; and wherein, if the 5-6 membered heteroaryl or 5-6 membered heterocyclyl contains a substitutable ring nitrogen atom, that ring nitrogen atom may be optionally substituted by C₁₋₆alkyl;
R^{3a} is independently, for each occurrence, selected from the group consisting of halogen, cyano, hydroxyl, C₁₋₆alkyl, phenyl, C₃₋₆cycloalkyl, 5-6 membered heterocyclyl, and —CO₂(C₁₋₆ alkyl), wherein the C₁₋₆alkyl may be optionally substituted on one or more available carbons by one, two, three, or more substituents each independently selected from halogen, hydroxyl, and phenyl; and wherein, if the 5-6 membered heterocyclyl contains a substitutable ring nitrogen atom, that ring nitrogen atom may be optionally substituted by C₁₋₆alkyl;
R^{4a} is independently, for each occurrence, selected from the group consisting of halogen, hydroxyl, and phenyl;
R^{5a} is independently, for each occurrence, selected from the group consisting of halogen, hydroxyl, and phenyl;
R^{6a} is independently, for each occurrence, halogen;
R^{8a} is independently, for each occurrence, selected from the group consisting of halogen, hydroxyl, and phenyl;
R^{e} is independently, for each occurrence, selected from the group consisting of halogen, hydroxyl, C₁₋₆alkyl, 3-6 membered cycloalkyl, 5-6 membered heterocyclyl, —N(R^{C})(R^{D}), and —S(O)₂C₁₋₆alkyl, wherein C₁₋₆alkyl or 5-6 membered heterocyclyl may be optionally substituted on one or more available carbons by one, two, three, or more substituents each independently selected from oxo and —N(R^{C})(R^{D}); and
R^{C} and R^{D} are each independently selected from hydrogen and C₁₋₆alkyl.

3. (canceled)

4. The compound of claim 2, wherein R⁴ is independently selected from the group consisting of chloro, fluoro, cyano, hydroxyl, oxo, CH₃, CF₃, —O—CH₃, —O—CH₂CH₃, —O—CH(CH₃)₂, —O—CH₂CH(CH₃)₂, —O—CH₂CF₃,

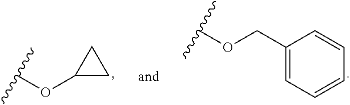 [SMILES: *OC1CC1 ; *OCc1ccccc1]

5. The compound of claim 2, wherein R⁴ is selected from the group consisting of chloro, fluoro, cyano, and CF₃.

6. The compound of claim 2, wherein R⁵ is selected from the group consisting of chloro, fluoro, cyano, hydroxyl, oxo, CH₃, CF₃, —O—CH₃, —O—CH₂CH₃, —O—CH(CH₃)₂, —O—CH₂CH(CH₃)₂, —O—CH₂CF₃,

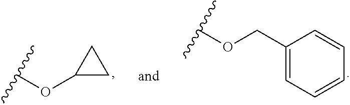 [SMILES: *OC1CC1 ; *OCc1ccccc1]

7. The compound of claim 6, wherein R⁵ is selected from the group consisting of CH₃, —O—CH₃, —O—CH₂—CH₃, —O—CH₂—CF₃, —O—CH₂—C(H)(CH₃)₂, —O—CH—(CH₃)₂,

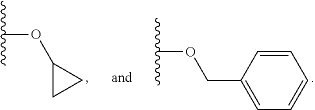 [SMILES: *OC1CC1 ; *OCc1ccccc1]

8. The compound of claim 2, wherein X is N.

9.-10. (canceled)

11. The compound of claim 2, wherein R⁶ is selected from the group consisting of halogen, C₁₋₆alkyl, and cyano.

12. The compound of claim 2, wherein R⁶ is selected from the group consisting of chloro, fluoro, cyano, and CH₃.

13. (canceled)

14. The compound of claim 2, wherein R⁷ is fluoro or chloro.

15.-16. (canceled)

17. The compound of claim 2, wherein R⁶ is fluoro, and R⁷ is fluoro.

18.-22. (canceled)

23. The compound of claim 2, wherein C is selected from the group consisting of 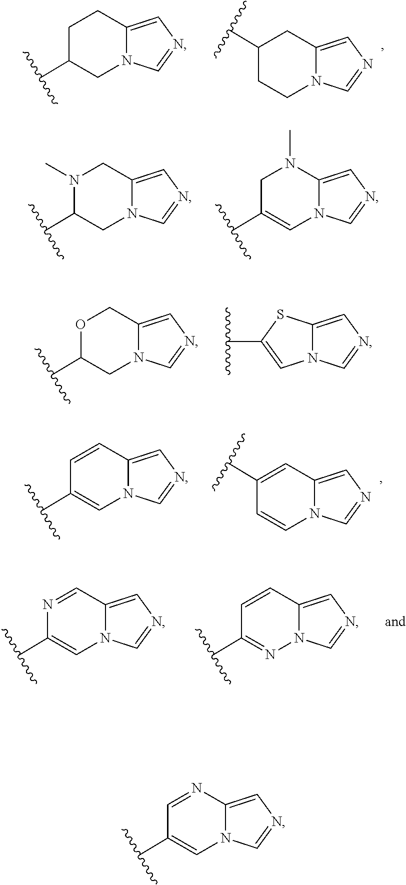
wherein C may be optionally substituted on one or more available carbons by one, two, three, or more independent R³ substituents selected from the group consisting of halogen, C₁₋₆alkyl, C₁₋₆alkoxyl, oxo, —C(O)N(R^{A})(R^{B}), —N(R^{A})(R^{B}), 5-10 membered heterocyclyl, and 5-10 membered heteroaryl, wherein the C₁₋₆alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl.

24. The compound of claim 2, wherein C is selected from the group consisting of 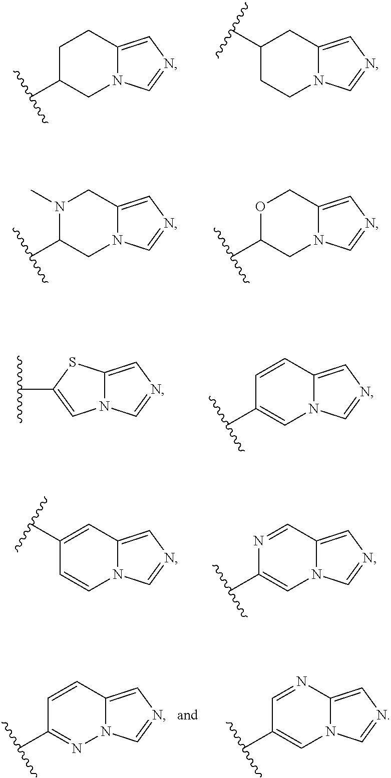
wherein C is substituted with one R³ substituent selected from the group consisting of halogen, C₁₋₆alkyl, C₁₋₆alkoxyl, oxo, —C(O)N(R^{A})(R^{B}), —N(R^{A})(R^{B}), 5-10 membered heterocyclyl, and 5-10 membered heteroaryl, wherein the C₁₋₆alkyl, 5-10 membered heteroaryl, or 5-10 membered heterocyclyl may be optionally substituted on one or more available carbons by one, two, three, or more substituents each independently selected from R^{3a}; and wherein, if the 5-10 membered heteroaryl contains a substitutable ring nitrogen atom, that ring nitrogen atom may be optionally substituted by C₁₋₆alkyl.

25. The compound of claim 2, wherein R³ is selected from the group consisting of C₁₋₆alkyl, —C(O)N(R^{A})(R^{B}), 5-10 membered heterocyclyl, and 5-10 membered heteroaryl, wherein the C₁₋₆alkyl and 5-10 membered heteroaryl may be optionally substituted on one or more available carbons by one, two, three, or more substituents, for each occurrence, independently selected from the group consisting of chloro, cyano, hydroxyl, CH₃, CF₃, —CH₂CH(CH₃)₂, —CH₂OH, —C(O)OCH₃, cyclopropyl, phenyl,

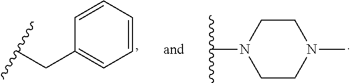

26. The compound of claim 2, wherein R^{A} is hydrogen.

27. The compound of claim 2, wherein R^{B} is selected from the group consisting of hydrogen, —CH₃, —CH₂CH₃, —CH(CH₃)₂, —CH₂CH(CH₃)₂, —CH₂CF₃, CH₂CH₂OH, —CH₂CH₂N(CH₃)₂, —(CH₂)₃N(CH₃)₂, —CH₂CH₂S(O)₂CH₃,

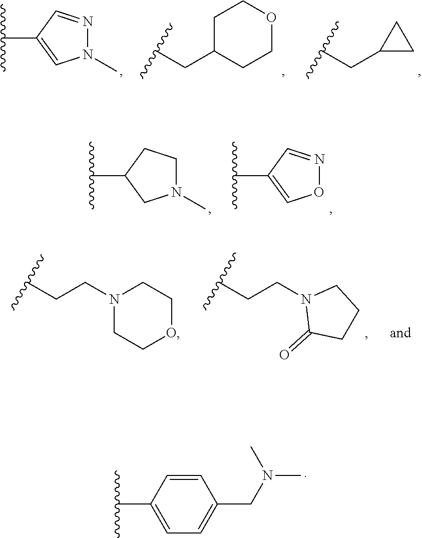

28. The compound of claim 2, wherein R³ is selected from the group consisting of —CH₂OH,

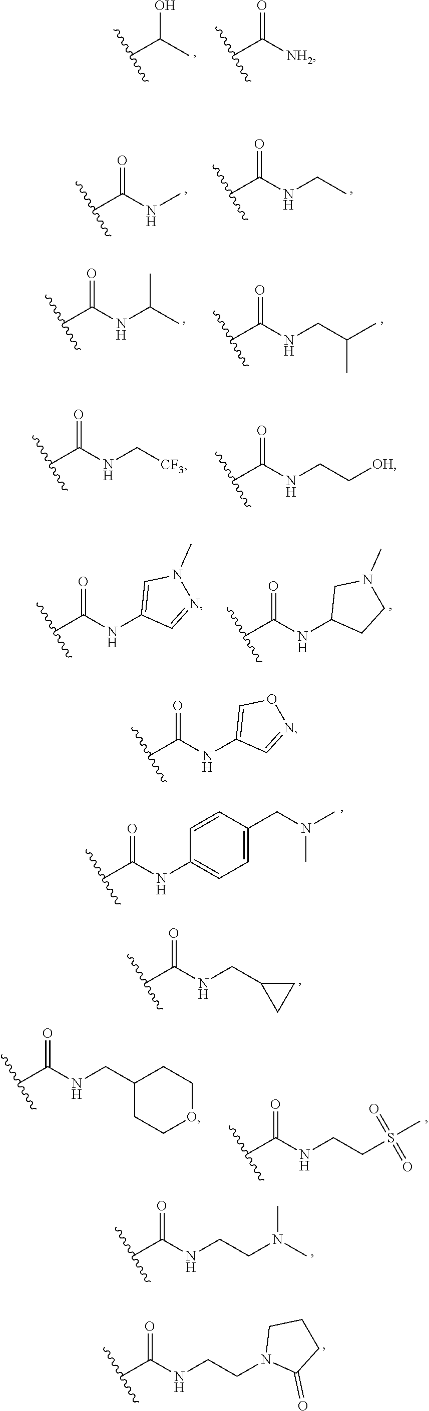

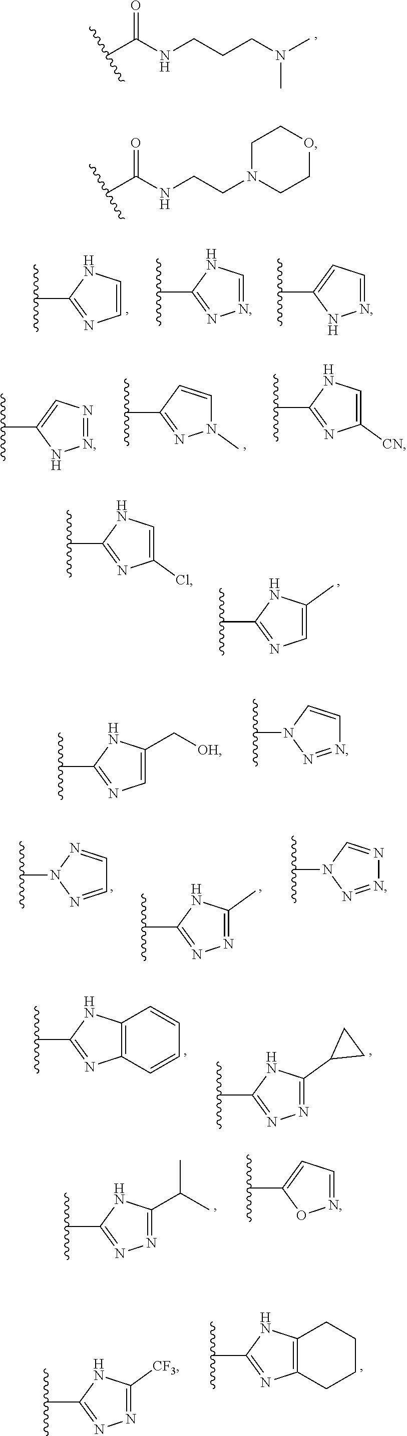

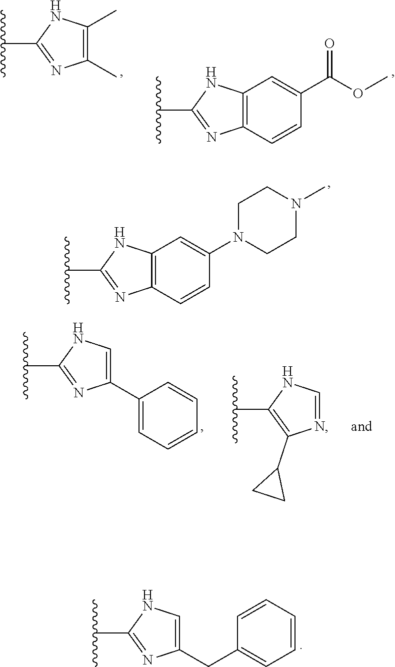

29. The compound of claim 2, wherein C is selected from the group consisting of

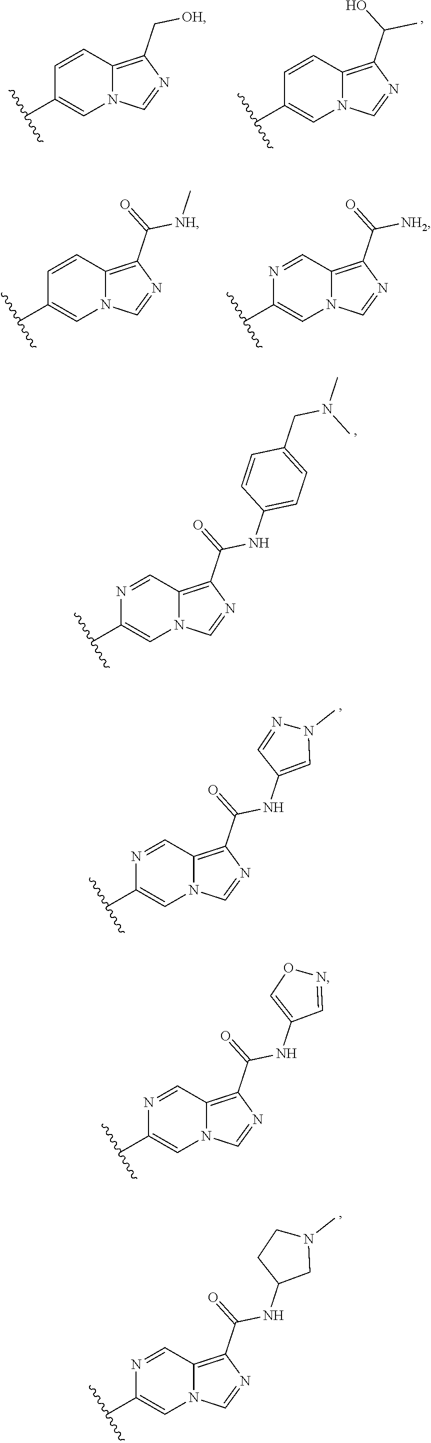

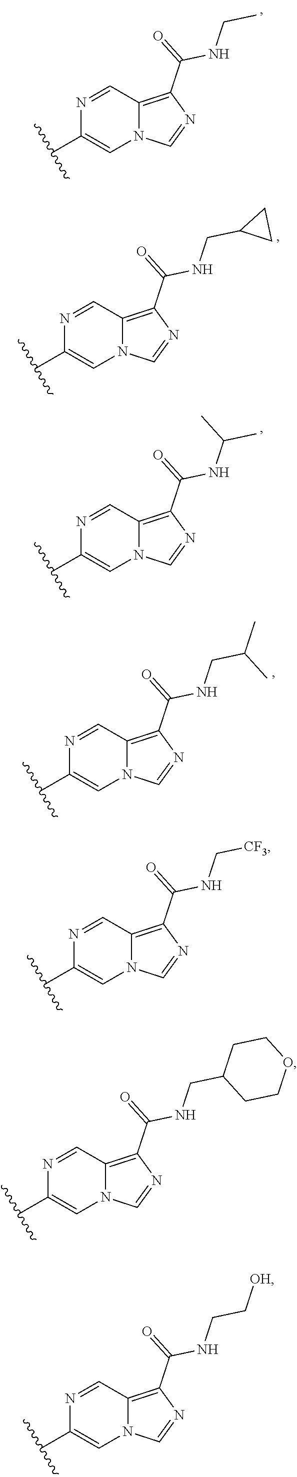

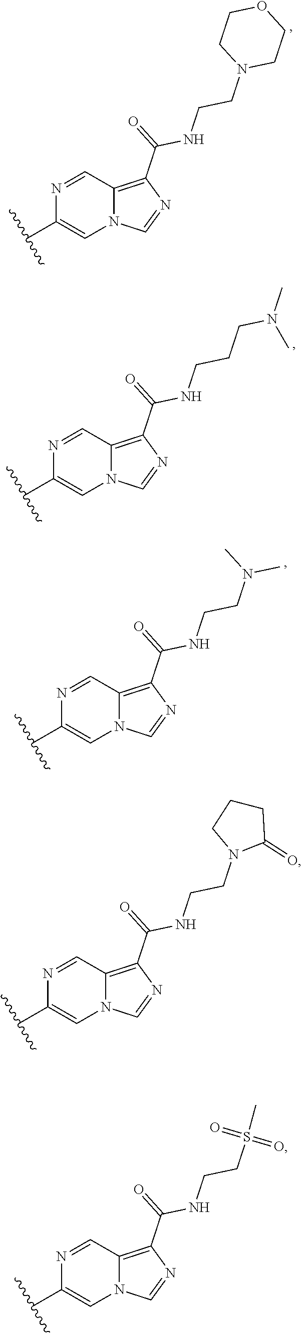 [SMILES: *c1cn2cnc(C(=O)NCCN3CCOCC3)c2cn1 ; *c1cn2cnc(C(=O)NCCCN(C)C)c2cn1 ; *c1cn2cnc(C(=O)NCCN(C)C)c2cn1 ; *c1cn2cnc(C(=O)NCCN3CCCC3=O)c2cn1 ; *c1cn2cnc(C(=O)NCCS(C)(=O)=O)c2cn1]

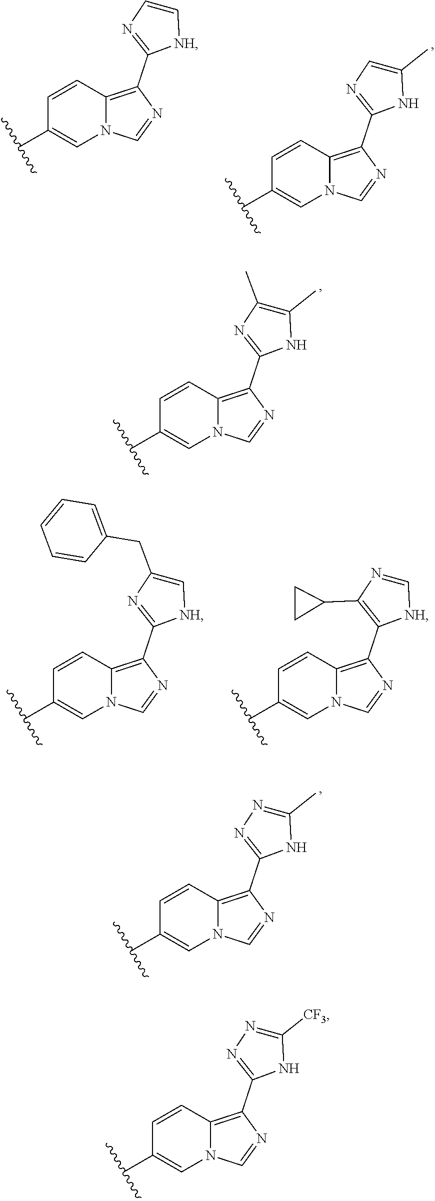

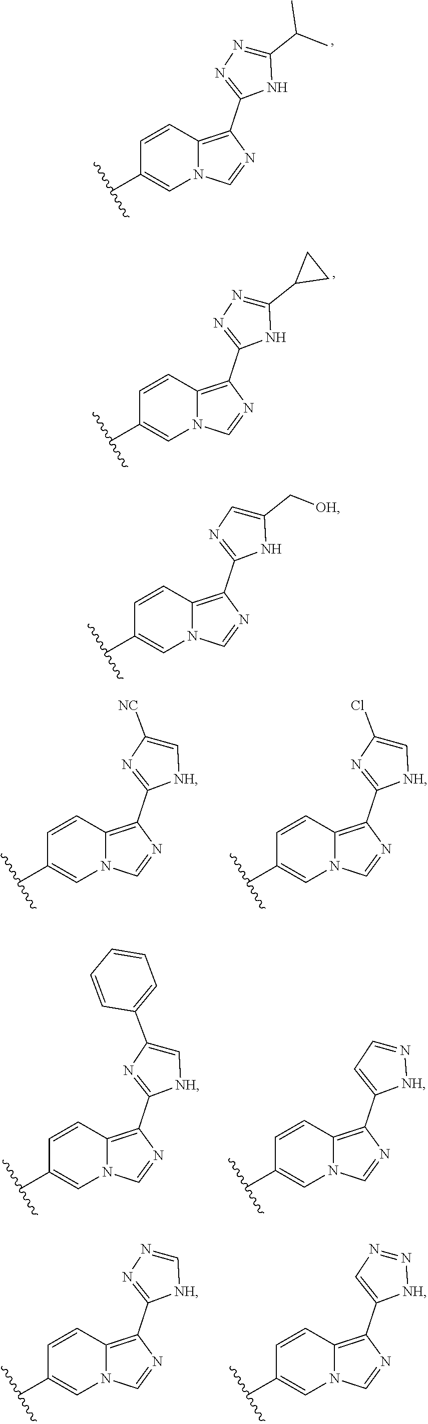

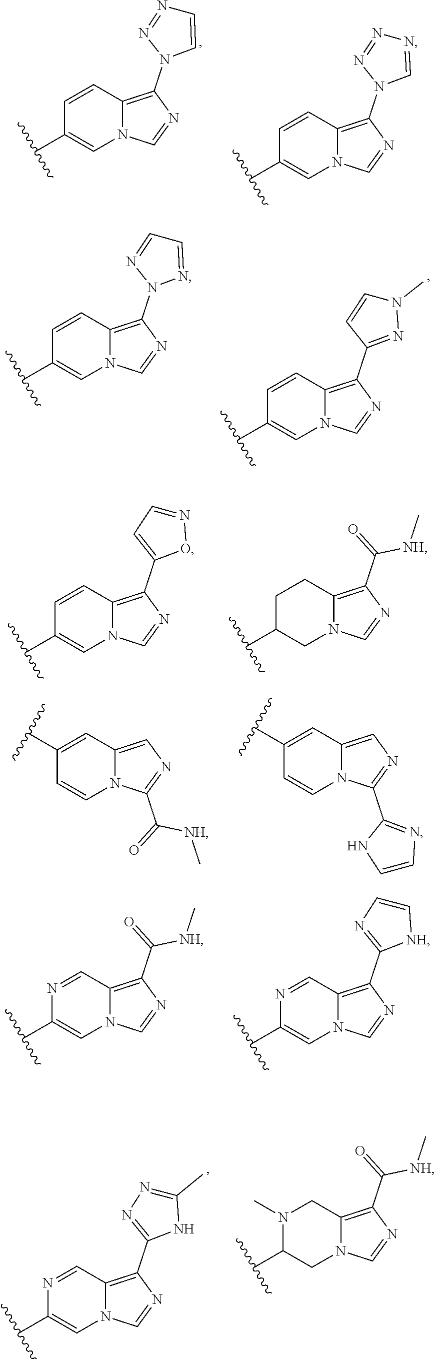

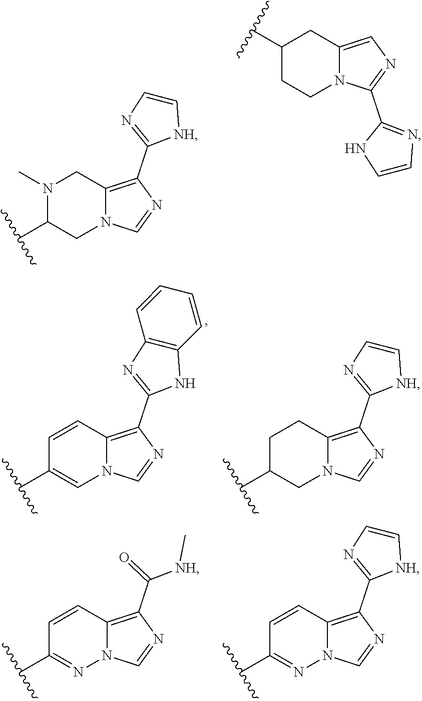

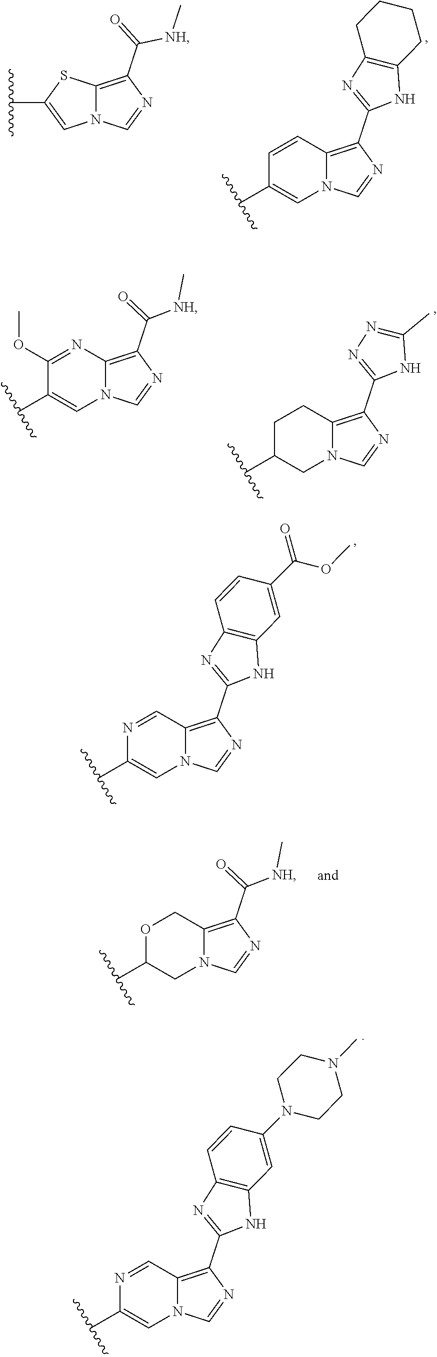

30.-35. (canceled)

36. The compound of claim 2 selected from the group consisting of:
6-[3-(5-chloro-2-methoxypyridine-3-sulfonamido)-2,6-difluorophenyl]-N-methylimidazo[1,5-a]pyridine-1-carboxamide;
5-chloro-N-[2,4-difluoro-3-[1-(1H-imidazol-2-yl)imidazo[1,5-a]pyridin-6-yl]phenyl]-2-methoxypyridine-3-sulfonamide;
6-[3-(5-chloro-2-methoxypyridine-3-sulfonamido)-2,6-difluorophenyl]-N-methylimidazo[1,5-a]pyrazine-1-carboxamide;
2-[3-(5-Chloro-2-methoxypyridine-3-sulfonamido)-2,6-difluorophenyl]-N-methylimidazo[1,5-b]pyridazine-5-carboxamide;
2-[3-(5-chloro-2-methylpyridine-3-sulfonamido)-2,6-difluorophenyl]-N-methylimidazo[1,5-b]pyridazine-5-carboxamide;
6-[2,6-difluoro-3-(5-fluoro-2-methylpyridine-3-sulfonamido)phenyl]-N-methylimidazo[1,5-a]pyridine-1-carboxamide;
6-[2,6-difluoro-3-(5-fluoro-2-methoxypyridine-3-sulfonamido)phenyl]-N-methylimidazo[1,5-a]pyridine-1-carboxamide;
6-[3-(5-cyano-2-methoxypyridine-3-sulfonamido)-2,6-difluorophenyl]-N-methylimidazo[1,5-a]pyridine-1-carboxamide;
6-[3-(5-cyano-2-methylpyridine-3-sulfonamido)-2,6-difluorophenyl]-N-methylimidazo[1,5-a]pyridine-1-carboxamide;
6-[3-(5-chloro-2-methoxypyridine-3-sulfonamido)-2,6-difluorophenyl]-N,5-dimethyl imidazo[1,5-a]pyridine-1-carboxamide;
2-[2,6-difluoro-3-(5-fluoro-2-methylpyridine-3-sulfonamido)phenyl]-N-methylimidazo[1,5-b]pyridazine-5-carboxamide;
6-[2,6-difluoro-3-[2-methoxy-5-(trifluoromethyl)pyridine-3-sulfonamido]phenyl]-N-methylimidazo[1,5-a]pyridine-1-carboxamide;
6-[3-(5-chloro-2-methoxypyridine-3-sulfonamido)-2,6-difluorophenyl]-5-fluoro-N-methylimidazo[1,5-a]pyridine-1-carboxamide;
6-[3-(5-chloro-2-methoxypyridine-3-sulfonamido)-2,6-difluorophenyl]-5-methoxy-N-methylimidazo[1,5-a]pyridine-1-carboxamide;
3-[3-(5-chloro-2-methoxypyridine-3-sulfonamido)-2,6-difluorophenyl]-N-methylimidazo[1,5-a]pyrimidine-8-carboxamide;
3-[3-(5-chloro-2-methoxypyridine-3-sulfonamido)-2,6-difluorophenyl]-2-methoxy-N-methylimidazo[1,5-a]pyrimidine-8-carboxamide;
N-[2,4-difluoro-3-[1-(1H-imidazol-2-yl)imidazo[1,5-a]pyridin-6-yl]phenyl]-5-fluoro-2-methoxypyridine-3-sulfonamide;
N-[2,4-difluoro-3-[1-(1H-imidazol-2-yl)imidazo[1,5-a]pyridin-6-yl]phenyl]-5-fluoro-2-methylpyridine-3-sulfonamide;
5-cyano-N-[2,4-difluoro-3-[1-(1H-imidazol-2-yl)imidazo[1,5-a]pyridin-6-yl]phenyl]-2-methoxypyridine-3-sulfonamide;
7-[3-(5-chloro-2-methoxypyridine-3-sulfonamido)-2,6-difluorophenyl]-8-fluoro-N-methylimidazo[1,5-a]pyridine-3-carboxamide;
3-[3-(5-chloro-2-methoxypyridine-3-sulfonamido)-2,6-difluorophenyl]-N,1-dimethyl-2-oxoimidazo[1,5-a]pyrimidine-8-carboxamide;
6-[3-(5-chloro-2-methoxypyridine-3-sulfonamido)-2,6-difluorophenyl]-N-methyl-5-(methylamino)imidazo[1,5-a]pyridine-1-carboxamide;
5-chloro-N-[2,4-difluoro-3-[1-(1H-imidazol-2-yl)imidazo[1,5-a]pyridin-6-yl]phenyl]-2-methylpyridine-3-sulfonamide;
(6R)-6-[3-(5-chloro-2-methoxypyridine-3-sulfonamido)-2,6-difluorophenyl]-N-methyl-5H,6H,7H,8H-imidazo[1,5-a]pyridine-1-carboxamide;
(6S)-6-[3-(5-chloro-2-methoxypyridine-3-sulfonamido)-2,6-difluorophenyl]-N-methyl-5H,6H,7H,8H-imidazo[1,5-a]pyridine-1-carboxamide;
5-chloro-N-[2,4-difluoro-3-[1-(4H-1,2,4-triazol-3-yl)imidazo[1,5-a]pyridin-6-yl]phenyl]-2-methoxypyridine-3-sulfonamide;
7-[3-(5-chloro-2-methoxypyridine-3-sulfonamido)-2,6-difluorophenyl]-N-methylimidazo[1,5-a]pyridine-3-carboxamide;
5-chloro-N-[2,4-difluoro-3-[1-(1H-imidazol-2-yl)imidazo[1,5-a]pyrazin-6-yl]phenyl]-2-methoxypyridine-3-sulfonamide;
5-chloro-N-[2,4-difluoro-3-[1-(2H-pyrazol-3-yl)imidazo[1,5-a]pyridin-6-yl]phenyl]-2-methoxypyridine-3-sulfonamide;
(6R)-6-[3-(5-chloro-2-methoxypyridine-3-sulfonamido)-2,6-difluorophenyl]-N,7-dimethyl-5H,6H,8H-imidazo[1,5-a]pyrazine-1-carboxamide;
(6S)-6-[3-(5-chloro-2-methoxypyridine-3-sulfonamido)-2,6-difluorophenyl]-N,7-dimethyl-5H,6H,8H-imidazo[1,5-a]pyrazine-1-carboxamide;
5-chloro-N-[2,4-difluoro-3-[1-(3H-1,2,3-triazol-4-yl)imidazo[1,5-a]pyridin-6-yl]phenyl]-2-methoxypyridine-3-sulfonamide;
5-chloro-N-[2,4-difluoro-3-[3-(1H-imidazol-2-yl)imidazo[1,5-a]pyridin-7-yl]phenyl]-2-methoxypyridine-3-sulfonamide;
N-[2,4-difluoro-3-[3-(1H-imidazol-2-yl)imidazo[1,5-a]pyridin-7-yl]phenyl]-5-fluoro-2-methoxypyridine-3-sulfonamide;
5-chloro-N-[2,4-difluoro-3-[1-(1-methylpyrazol-3-yl)imidazo[1,5-a]pyridin-6-yl]phenyl]-2-methoxypyridine-3-sulfonamide;
5-chloro-N-[2-cyano-4-fluoro-3-[1-(1H-imidazol-2-yl)imidazo[1,5-a]pyridin-6-yl]phenyl]-2-methoxypyridine-3-sulfonamide;
5-cyano-N-[2,4-difluoro-3-[3-(1H-imidazol-2-yl)imidazo[1,5-a]pyridin-7-yl]phenyl]-2-methoxypyridine-3-sulfonamide;
5-chloro-N-[2,4-difluoro-3-[8-fluoro-3-(1H-imidazol-2-yl)imidazo[1,5-a]pyridin-7-yl]phenyl]-2-methoxypyridine-3-sulfonamide;
5-cyano-N-[2,4-difluoro-3-[8-fluoro-3-(1H-imidazol-2-yl)imidazo[1,5-a]pyridin-7-yl]phenyl]-2-methoxypyridine-3-sulfonamide;
5-chloro-N-(3-(1-(4-cyano-1H-imidazol-2-yl)imidazo[1,5-a]pyridin-6-yl)-2,4-difluorophenyl)-2-methoxypyridine-3-sulfonamide;
N-[3-[1-(4-cyano-1H-imidazol-2-yl)imidazo[1,5-a]pyridin-6-yl]-2,4-difluorophenyl]-5-fluoro-2-methylpyridine-3-sulfonamide;
N-[3-[1-(4-chloro-1H-imidazol-2-yl)imidazo[1,5-a]pyridin-6-yl]-2,4-difluorophenyl]-5-fluoro-2-methoxypyridine-3-sulfonamide;
N-[2,4-difluoro-3-[1-(1-methylpyrazol-3-yl)imidazo[1,5-a]pyridin-6-yl]phenyl]-5-fluoro-2-methoxypyridine-3-sulfonamide;
N-[2,4-difluoro-3-[8-fluoro-3-(1H-imidazol-2-yl)imidazo[1,5-a]pyridin-7-yl]phenyl]-5-fluoro-2-methoxypyridine-3-sulfonamide;
5-chloro-N-[2,4-difluoro-3-[1-(4-methyl-3H-imidazol-2-yl)imidazo[1,5-a]pyridine-6-yl]phenyl]-2-methoxypyridine-3-sulfonamide;
5-chloro-N-(2,4-difluoro-3-[1-[4-(hydroxymethyl)-3H-imidazol-2-yl]imidazo[1,5-a pyridin-6-yl]phenyl)-2-methoxypyridine-3-sulfonamide;
N-[2,4-difluoro-3-[1-(4-methyl-3H-imidazol-2-yl)imidazo[1,5-a]pyridin-6-yl]phenyl]-5-fluoro-2-methylpyridine-3-sulfonamide;
N-(2,4-difluoro-3-[1-[4-(hydroxymethyl)-3H-imidazol-2-yl]imidazo[1,5-a]pyridin-6-yl]phenyl)-5-fluoro-2-methylpyridine-3-sulfonamide;
5-chloro-N-[3-[1-(4-chloro-1H-imidazol-2-yl)imidazo[1,5-a]pyridin-6-yl]-2,4-difluorophenyl]-2-methoxypyridine-3-sulfonamide;
5-cyano-N-[2,4-difluoro-3-[1-(1-methylpyrazol-3-yl)imidazo[1,5-a]pyridin-6-yl]phenyl]-2-methoxypyridine-3-sulfonamide;
N-[2,4-difluoro-3-[1-(1-methylpyrazol-3-yl)imidazo[1,5-a]pyridin-6-yl]phenyl]-5-fluoro-2-methylpyridine-3-sulfonamide;
5-chloro-N-[2,4-difluoro-3-[1-(1,2,3-triazol-1-yl)imidazo[1,5-a]pyridin-6-yl]phenyl]-2-methoxypyridine-3-sulfonamide;
5-chloro-N-[2,4-difluoro-3-[1-(1,2,3-triazol-2-yl)imidazo[1,5-a]pyridin-6-yl]phenyl]-2-methoxypyridine-3-sulfonamide;
5-chloro-N-[2,4-difluoro-3-[1-(4-methyl-3H-imidazol-2-yl)imidazo[1,5-a]pyridin-6-yl]phenyl]-2-methylpyridine-3-sulfonamide;
5-chloro-N-[2,4-difluoro-3-[1-(5-methyl-4H-1,2,4-triazol-3-yl)imidazo[1,5-a]pyridin-6-yl]phenyl]-2-methoxypyridine-3-sulfonamide;
N-[2,4-difluoro-3-[1-(5-methyl-4H-1,2,4-triazol-3-yl)imidazo[1,5-a]pyridin-6-yl]phenyl]-5-fluoro-2-methoxypyridine-3-sulfonamide;
5-cyano-N-[2,4-difluoro-3-[1-(5-methyl-4H-1,2,4-triazol-3-yl)imidazo[1,5-a]pyridin-6-yl]phenyl]-2-methoxypyridine-3-sulfonamide;
N-[2,4-difluoro-3-[1-(5-methyl-4H-1,2,4-triazol-3-yl)imidazo[1,5-a]pyridin-6-yl]phenyl]-5-fluoro-2-methylpyridine-3-sulfonamide;
5-chloro-N-[2,4-difluoro-3-[1-(5-methyl-4H-1,2,4-triazol-3-yl)imidazo[1,5-a]pyridin-6-yl]phenyl]-2-methylpyridine-3-sulfonamide;
5-chloro-N-(2,4-difluoro-3-(1-(1-methyl-1H-pyrazol-3-yl)imidazo[1,5-a]pyridin-6-yl)phenyl)-2-methylpyridine-3-sulfonamide;
5-chloro-N-[2,4-difluoro-3-[1-(1,2,3,4-tetrazol-1-yl)imidazo[1,5-a]pyridin-6-yl]phenyl]-2-methoxypyridine-3-sulfonamide;
(R)—N-(3-(3-(1H-imidazol-2-yl)-5,6,7,8-tetrahydroimidazo[1,5-a]pyridin-7-yl)-2,4-difluorophenyl)-5-chloro-2-methoxypyridine-3-sulfonamide;
(S)—N-(3-(3-(1H-imidazol-2-yl)-5,6,7,8-tetrahydroimidazo[1,5-a]pyridin-7-yl)-2,4-difluorophenyl)-5-chloro-2-methoxypyridine-3-sulfonamide;
5-chloro-N-[2,4-difluoro-3-[1-(4H-1,2,4-triazol-3-yl)imidazo[1,5-a]pyridin-6-yl]phenyl]-2-methylpyridine-3-sulfonamide;
N-[3-[1-(1H-1,3-benzodiazol-2-yl)imidazo[1,5-a]pyridin-6-yl]-2,4-difluorophenyl]-5-cyano-2-methoxypyridine-3-sulfonamide;
(R)-6-(2,6-difluoro-3-((5-fluoro-2-methoxypyridine)-3-sulfonamido)phenyl)-N-methyl-5,6,7,8-tetrahydroimidazo[1,5-a]pyridine-1-carboxamide;
(S)-6-(2,6-difluoro-3-((5-fluoro-2-methoxypyridine)-3-sulfonamido)phenyl)-N-methyl-5,6,7,8-tetrahydroimidazo[1,5-a]pyridine-1-carboxamide;
(R)-6-(3-((5-cyano-2-methoxypyridine)-3-sulfonamido)-2,6-difluorophenyl)-N-methyl-5,6,7,8-tetrahydroimidazo[1,5-a]pyridine-1-carboxamide;
(S)-6-(3-((5-cyano-2-methoxypyridine)-3-sulfonamido)-2,6-difluorophenyl)-N-methyl-5,6,7,8-tetrahydroimidazo[1,5-a]pyridine-1-carboxamide;
5-cyano-N-[2,4-difluoro-3-[1-(1H-imidazol-2-yl)imidazo[1,5-a]pyrazin-6-yl]phenyl]-2-methoxypyridine-3-sulfonamide;
N-[2,4-difluoro-3-[1-(4H-1,2,4-triazol-3-yl)imidazo[1,5-a]pyridin-6-yl]phenyl]-5-fluoro-2-methoxypyridine-3-sulfonamide;
5-cyano-N-[2,4-difluoro-3-[1-(4H-1,2,4-triazol-3-yl)imidazo[1,5-a]pyridin-6-yl]phenyl]-2-methoxypyridine-3-sulfonamide;
N-[2,4-difluoro-3-[1-(4H-1,2,4-triazol-3-yl)imidazo[1,5-a]pyridin-6-yl]phenyl]-5-fluoro-2-methylpyridine-3-sulfonamide;
5-chloro-N-[2,4-difluoro-3-[(6R)-1-(1H-imidazol-2-yl)-7-methyl-5H,6H,8H-imidazo[1,5-a]pyrazin-6-yl]phenyl]-2-methoxypyridine-3-sulfonamide;
5-chloro-N-[2,4-difluoro-3-[(6S)-1-(1H-imidazol-2-yl)-7-methyl-5H,6H,8H-imidazo[1,5-a]pyrazin-6-yl]phenyl]-2-methoxypyridine-3-sulfonamide;
N-[2,4-difluoro-3-[1-(1,2,3,4-tetrazol-1-yl)imidazo[1,5-a]pyridin-6-yl]phenyl]-5-fluoro-2-methoxypyridine-3-sulfonamide;
5-cyano-N-[2,4-difluoro-3-[1-(1,2,3,4-tetrazol-1-yl)imidazo[1,5-a]pyridin-6-yl]phenyl]-2-methoxypyridine-3-sulfonamide;
N-[2,4-difluoro-3-[1-(1,2,3,4-tetrazol-1-yl)imidazo[1,5-a]pyridin-6-yl]phenyl]-5-fluoro-2-methylpyridine-3-sulfonamide;
5-chloro-N-[2,4-difluoro-3-[1-(1,2,3,4-tetrazol-1-yl)imidazo[1,5-a]pyridin-6-yl]phenyl]-2-methylpyridine-3-sulfonamide;
N-[2,4-difluoro-3-[1-(1H-imidazol-2-yl)imidazo[1,5-a]pyrazin-6-yl]phenyl]-5-fluoro-2-methoxypyridine-3-sulfonamide;
5-chloro-N-[2,4-difluoro-3-[(6R)-1-(1H-imidazol-2-yl)-5H,6H,7H,8H-imidazo[1,5-a]pyridin-6-yl]phenyl]-2-methoxypyridine-3-sulfonamide;
5-chloro-N-[2,4-difluoro-3-[(6S)-1-(1H-imidazol-2-yl)-5H,6H,7H,8H-imidazo[1,5-a]pyridin-6-yl]phenyl]-2-methoxypyridine-3-sulfonamide;
N-[2,4-difluoro-3-[(6R)-1-(1H-imidazol-2-yl)-5H,6H,7H,8H-imidazo[1,5-a]pyridin-6-yl]phenyl]-5-fluoro-2-methylpyridine-3-sulfonamide;
N-[2,4-difluoro-3-[(6S)-1-(1H-imidazol-2-yl)-5H,6H,7H,8H-imidazo[1,5-a]pyridin-6-yl]phenyl]-5-fluoro-2-methylpyridine-3-sulfonamide;
N-[2,4-difluoro-3-[(6R)-1-(1H-imidazol-2-yl)-5H,6H,7H,88H-imidazo[1,5-a]pyridin-6-yl]phenyl]-5-fluoro-2-methoxypyridine-3-sulfonamide;
N-[2,4-difluoro-3-[(6S)-1-(1H-imidazol-2-yl)-5H,6H,7H,8H-imidazo[1,5-a]pyridin-6-yl]phenyl]-5-fluoro-2-methoxypyridine-3-sulfonamide;
5-cyano-N-[2,4-difluoro-3-[(6R)-1-(1H-imidazol-2-yl)-5H,6H,7H,8H-imidazo[1,5-a]pyridin-6-yl]phenyl]-2-methoxypyridine-3-sulfonamide;
5-cyano-N-[2,4-difluoro-3-[(6S)-1-(1H-imidazol-2-yl)-5H,6H,7H,88H-imidazo[1,5-a]pyridin-6-yl]phenyl]-2-methoxypyridine-3-sulfonamide;
5-chloro-N-[2,4-difluoro-3-[1-(1H-imidazol-2-yl)imidazo[1,5-a]pyrazin-6-yl]phenyl]-2-methylpyridine-3-sulfonamide;
5-chloro-N-[2,4-difluoro-3-[(6R)-1-(1H-imidazol-2-yl)-5H,6H,7H,8H-imidazo[1,5-a]pyridin-6-yl]phenyl]-2-methylpyridine-3-sulfonamide;
5-chloro-N-[2,4-difluoro-3-[(6S)-1-(1H-imidazol-2-yl)-5H,6H,7H,8H-imidazo[1,5-a]pyridin-6-yl]phenyl]-2-methylpyridine-3-sulfonamide;
5-chloro-N-[2,4-difluoro-3-[5-(1H-imidazol-2-yl)imidazo[1,5-b]pyridazin-2-yl]phenyl]-2-methoxypyridine-3-sulfonamide;
(R)-6-(2,6-difluoro-3-((5-fluoro-2-methylpyridine)-3-sulfonamido)phenyl)-N-methyl-5,6,7,8-tetrahydroimidazo[1,5-a]pyridine-1-carboxamide;
(S)-6-(2,6-difluoro-3-((5-fluoro-2-methylpyridine)-3-sulfonamido)phenyl)-N-methyl-5,6,7,8-tetrahydroimidazo[1,5-a]pyridine-1-carboxamide;
5-chloro-N-[3-[1-(5-cyclopropyl-4H-1,2,4-triazol-3-yl)imidazo[1,5-a]pyridin-6-yl]-2,4-difluorophenyl]-2-methoxypyridine-3-sulfonamide;
5-chloro-N-[2,4-difluoro-3-[1-(5-isopropyl-4H-1,2,4-triazol-3-yl)imidazo[1,5-a]pyridin-6-yl]phenyl]-2-methoxypyridine-3-sulfonamide;
(R)-6-(3-((5-chloro-2-methylpyridine)-3-sulfonamido)-2,6-difluorophenyl)-N-methyl-5,6,7,8-tetrahydroimidazo[1,5-a]pyridine-1-carboxamide;
(S)-6-(3-((5-chloro-2-methylpyridine)-3-sulfonamido)-2,6-difluorophenyl)-N-methyl-5,6,7,8-tetrahydroimidazo[1,5-a]pyridine-1-carboxamide;
6-[2,6-difluoro-3-(5-fluoro-2-methoxypyridine-3-sulfonamido)phenyl]-N-methylimidazo[1,5-a]pyrazine-1-carboxamide;
6-[2,6-difluoro-3-(5-fluoro-2-methylpyridine-3-sulfonamido)phenyl]-N-methylimidazo[1,5-a]pyrazine-1-carboxamide;
6-[3-(5-cyano-2-methoxypyridine-3-sulfonamido)-2,6-difluorophenyl]-N-methylimidazo[1,5-a]pyrazine-1-carboxamide;
6-[3-(5-chloro-2-methylpyridine-3-sulfonamido)-2,6-difluorophenyl]-N-methylimidazo[1,5-a]pyrazine-1-carboxamide;
5-chloro-N-(2,4-difluoro-3-(1-(hydroxymethyl)imidazo[1,5-a]pyridin-6-yl)phenyl)-2-methoxypyridine-3-sulfonamide;
5-chloro-N-[2,4-difluoro-3-[5-(1H-imidazol-2-yl)imidazo[1,5-b]pyridazin-2-yl]phenyl]-2-methylpyridine-3-sulfonamide;
(5S,6R)-6-[3-(5-chloro-2-methoxypyridine-3-sulfonamido)-2,6-difluorophenyl]-N,5-dimethyl-5H,6H,7H,8H-imidazo[1,5-a]pyridine-1-carboxamide;
(5R,6S)-6-[3-(5-chloro-2-methoxypyridine-3-sulfonamido)-2,6-difluorophenyl]-N,5-dimethyl-5H,6H,7H,8H-imidazo[1,5-a]pyridine-1-carboxamide;
5-cyano-N-[2,4-difluoro-3-[5-(1H-imidazol-2-yl)imidazo[1,5-b]pyridazin-2-yl]phenyl]-2-methoxypyridine-3-sulfonamide;
N-[2,4-difluoro-3-[5-(1H-imidazol-2-yl)imidazo[1,5-b]pyridazin-2-yl]phenyl]-5-fluoro-2-methoxypyridine-3-sulfonamide;
5-chloro-N-(2,4-difluoro-3-(1-(isoxazol-5-yl)imidazo[1,5-a]pyridin-6-yl)phenyl)-2-methoxypyridine-3-sulfonamide;
5-chloro-N-[2,4-difluoro-3-[1-(1-hydroxyethyl)imidazo[1,5-a]pyridin-6-yl]phenyl]-2-methoxypyridine-3-sulfonamide;
6-(2-chloro-3-((5-chloro-2-methoxypyridine)-3-sulfonamido)-6-fluorophenyl)-N-methylimidazo[1,5-a]pyridine-1-carboxamide;
5-chloro-N-(2,4-difluoro-3-[1-[5-(trifluoromethyl)-4H-1,2,4-triazol-3-yl]imidazo[1,5-a]pyridin-6-yl]phenyl)-2-methoxypyridine-3-sulfonamide;
5-chloro-N-[2-chloro-4-fluoro-3-[1-(1H-imidazol-2-yl)imidazo[1,5-a]pyridin-6-yl]phenyl]-2-methoxypyridine-3-sulfonamide;
5-chloro-N-[2,4-difluoro-3-[5-fluoro-1-(1H-imidazol-2-yl)imidazo[1,5-a]pyridin-6-yl]phenyl]-2-methoxypyridine-3-sulfonamide;
5-chloro-N-[2,4-difluoro-3-[5-fluoro-1-(2H-pyrazol-3-yl)imidazo[1,5-a]pyridin-6-yl]phenyl]-2-methoxypyridine-3-sulfonamide;
6-[2-chloro-3-(5-chloro-2-methoxypyridine-3-sulfonamido)-6-fluorophenyl]-N-methylimidazo[1,5-a]pyrazine-1-carboxamide;
N-[2,4-difluoro-3-[5-fluoro-1-(1H-imidazol-2-yl)imidazo[1,5-a]pyridin-6-yl]phenyl]-5-fluoro-2-methoxypyridine-3-sulfonamide;
N-[2,4-difluoro-3-[5-fluoro-1-(1H-imidazol-2-yl)imidazo[1,5-a]pyridin-6-yl]phenyl]-5-fluoro-2-methylpyridine-3-sulfonamide;
5-chloro-N-[2,4-difluoro-3-[5-fluoro-1-(1H-imidazol-2-yl)imidazo[1,5-a]pyridine-6-yl]phenyl]-2-methylpyridine-3-sulfonamide;
N-[3-[1-(1H-1,3-benzodiazol-2-yl)imidazo[1,5-a]pyrazin-6-yl]-2,4-difluorophenyl]-5-chloro-2-methoxypyridine-3-sulfonamide;
N-[3-[1-(1H-1,3-benzodiazol-2-yl)imidazo[1,5-a]pyrazin-6-yl]-2,4-difluorophenyl]-5-fluoro-2-methoxypyridine-3-sulfonamide;
N-[3-[1-(1H-1,3-benzodiazol-2-yl)imidazo[1,5-a]pyrazin-6-yl]-2,4-difluorophenyl]-5-cyano-2-methoxypyridine-3-sulfonamide;
N-[3-[1-(1H-1,3-benzodiazol-2-yl)imidazo[1,5-a]pyrazin-6-yl]-2,4-difluorophenyl]-5-chloro-2-methylpyridine-3-sulfonamide;
N-[3-[1-(1H-1,3-benzodiazol-2-yl)imidazo[1,5-a]pyrazin-6-yl]-2,4-difluorophenyl]-5-fluoro-2-methylpyridine-3-sulfonamide;
N-[3-[1-(4-chloro-1H-imidazol-2-yl)-5-fluoroimidazo[1,5-a]pyridin-6-yl]-2,4-difluorophenyl]-5-fluoro-2-methoxypyridine-3-sulfonamide;
5-chloro-N-[3-[1-(4-chloro-1H-imidazol-2-yl)-5-fluoroimidazo[1,5-a]pyridin-6-yl]-2,4-difluorophenyl]-2-methoxypyridine-3-sulfonamide;
6-[3-(5-chloro-2-methoxypyridine-3-sulfonamido)-2-cvano-6-fluorophenyl]-N-methylimidazo[1,5-a]pyrazine-1-carboxamide;
N-[2,4-difluoro-3-[1-(1,2-oxazol-5-yl)imidazo[1,5-a]pyridin-6-yl]phenyl]-5-fluoro-2-methoxypyridine-3-sulfonamide;
5-cyano-N-[2,4-difluoro-3-[1-(1,2-oxazol-5-yl)imidazo[1,5-a]pyridin-6-yl]phenyl]-2-methoxypyridine-3-sulfonamide;
2-[3-(5-chloro-2-methoxypyridine-3-sulfonamido)-2,6-difluorophenyl]-N-methylimidazo[4,3-b][1,3]thiazole-7-carboxamide;
5-chloro-N-[2,4-difluoro-3-[1-(1,2-oxazol-5-yl)imidazo[1,5-a]pyridin-6-yl]phenyl]-2-methylpyridine-3-sulfonamide;
6-[3-(5-chloro-2-methoxypyridine-3-sulfonamido)-6-fluoro-2-methylphenyl]-N-methylimidazo[1,5-a]pyridine-1-carboxamide;
5-cyano-N-[2,4-difluoro-3-[1-(4,5,6,7-tetrahydro-1H-1,3-benzodiazol-2-yl)imidazo[1,5-a]pyridin-6-yl]phenyl]-2-methoxypyridine-3-sulfonamide;
5-chloro-N-[2,4-difluoro-3-[1-(5-methyl-4H-1,2,4-triazol-3-yl)imidazo[1,5-a]pyrazin-6-yl]phenyl]-2-methoxypyridine-3-sulfonamide;
N-[2,4-difluoro-3-[1-(1,2-oxazol-5-yl)imidazo[1,5-a]pyridin-6-yl]phenyl]-5-fluoro-2-methylpyridine-3-sulfonamide;
5-cyano-N-[3-[1-(4,5-dimethyl-1H-imidazol-2-yl)imidazo[1,5-a]pyridin-6-yl]-2,4-difluorophenyl]-2-methoxypyridine-3-sulfonamide;
N-[2,4-difluoro-3-[1-(5-methyl-4H-1,2,4-triazol-3-yl)imidazo[1,5-a]pyrazin-6-yl]phenyl]-5-fluoro-2-methoxypyridine-3-sulfonamide;
5-cyano-N-[2,4-difluoro-3-[1-(5-methyl-4H-1,2,4-triazol-3-yl)imidazo[1,5-a]pyrazin-6-yl]phenyl]-2-methoxypyridine-3-sulfonamide;
N-[2,4-difluoro-3-[1-(5-methyl-4H-1,2,4-triazol-3-yl)imidazo[1,5-a]pyrazin-6-yl]phenyl]-5-fluoro-2-methylpyridine-3-sulfonamide;
5-chloro-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methoxypyridine-3-sulfonamide;
5-chloro-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methoxypyridine-3-sulfonamide;
N-[2,4-difluoro-3-[(6S)-1-(5-methyl-4H-1,2,4-triazol-3-yl)-5H,6H,7H,8H-imidazo[1,5-a]pyridin-6-yl]phenyl]-5-fluoro-2-methoxypyridine-3-sulfonamide;
N-[2,4-difluoro-3-[(6R)-1-(5-methyl-4H-1,2,4-triazol-3-yl)-5H,6H,7H,8H-imidazo[1,5-a]pyridin-6-yl]phenyl]-5-fluoro-2-methoxypyridine-3-sulfonamide;
5-chloro-N-[2,4-difluoro-3-[1-(5-methyl-4H-1,2,4-triazol-3-yl)imidazo[1,5-a]pyrazin-6-yl]phenyl]-2-methylpyridine-3-sulfonamide;
N-[2,4-difluoro-3-[(6S)-1-(5-methyl-4H-1,2,4-triazol-3-yl)-5H,6H,7H,8H-imidazo[1,5-a]pyridin-6-yl]phenyl]-5-fluoro-2-methylpyridine-3-sulfonamide;
N-[2,4-difluoro-3-[(6R)-1-(5-methyl-4H-1,2,4-triazol-3-yl)-5H,6H,7H,8H-imidazo[1,5-a]pyridin-6-yl]phenyl]-5-fluoro-2-methylpyridine-3-sulfonamide;
5-cyano-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methoxypyridine-3-sulfonamide;
5-cyano-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methoxypyridine-3-sulfonamide;
5-chloro-N-[2,4-difluoro-3-[(6S)-1-(5-methyl-4H-1,2,4-triazol-3-yl)-5H,6H,7H,8H-imidazo[1,5-a]pyridin-6-yl]phenyl]-2-methylpyridine-3-sulfonamide;
5-chloro-N-[2,4-difluoro-3-[(6R)-1-(5-methyl-4H-1,2,4-triazol-3-yl)-5H,6H,7H,8H-imidazo[1,5-a]pyridin-6-yl]phenyl]-2-methylpyridine-3-sulfonamide;
6-[6-chloro-3-(5-chloro-2-methoxypyridine-3-sulfonamido)-2-fluorophenyl]-N-methylimidazo[1,5-a]pyridine-1-carboxamide;
2-[6-chloro-3-(5-chloro-2-methylpyridine-3-sulfonamido)-2-fluorophenyl]-N-methylimidazo[1,5-b]pyridazine-5-carboxamide;
2-[2-chloro-3-(5-chloro-2-methylpyridine-3-sulfonamido)-6-fluorophenyl]-N-methylimidazo[1,5-b]pyridazine-5-carboxamide;
6-(6-chloro-3-((5-chloro-2-methoxypyridine)-3-sulfonamido)-2-fluorophenyl)-N-methylimidazo[1,5-a]pyrazine-1-carboxamide;
N-[4-chloro-2-fluoro-3-[1-(1H-imidazol-2-yl)imidazo[1,5-a]pyrazin-6-yl]phenyl]-5-fluoro-2-methoxypyridine-3-sulfonamide;
5-chloro-N-(3-(1-(4,5-dimethyl-1H-imidazol-2-yl)imidazo[1,5-a]pyridin-6-yl)-2,4-difluorophenyl)-2-methoxypyridine-3-sulfonamide;
N-(3-(1-(4,5-dimethyl-1H-imidazol-2-yl)imidazo[1,5-a]pyridin-6-yl)-2,4-difluorophenyl)-5-fluoro-2-methoxypyridine-3-sulfonamide;
N-(3-(1-(4,5-dimethyl-1H-imidazol-2-yl)imidazo[1,5-a]pyridin-6-yl)-2,4-difluorophenyl)-5-fluoro-2-methylpyridine-3-sulfonamide;
5-chloro-N-(3-(1-(4,5-dimethyl-1H-imidazol-2-yl)imidazo[1,5-a]pyridin-6-yl)-2,4-difluorophenyl)-2-methylpyridine-3-sulfonamide;
5-chloro-N-(2,4-difluoro-3-(1-(4,5,6,7-tetrahydro-1H-benzo[d]imidazol-2-yl) imidazo[1,5-a]pyridin-6-yl)phenyl)-2-methoxypyridine-3-sulfonamide;
N-(2,4-difluoro-3-(1-(4,5,6,7-tetrahydro-1H-benzo[d]imidazol-2-yl)imidazo[1,5-a]pyridin-6-yl)phenyl)-5-fluoro-2-methoxypyridine-3-sulfonamide;
N-(2,4-difluoro-3-(1-(4,5,6,7-tetrahydro-1H-benzo[d]imidazol-2-yl) imidazo[1,5-a]pyridin-6-yl)phenyl)-5-fluoro-2-methylpyridine-3-sulfonamide;
5-chloro-N-(2,4-difluoro-3-(1-(4,5,6,7-tetrahydro-1H-benzo[d]imidazol-2-yl)imidazo[1,5-a]pyridin-6-yl)phenyl)-2-methylpyridine-3-sulfonamide;
N-[2-chloro-4-fluoro-3-[1-(1H-imidazol-2-yl)imidazo[1,5-a]pyrazin-6-yl]phenyl]-5-fluoro-2-methoxypyridine-3-sulfonamide;
(6R)-6-[2-chloro-6-fluoro-3-(5-fluoro-2-methoxypyridine-3-sulfonamido)phenyl]-N-methyl-5H,6H,7H,8H-imidazo[1,5-a]pyridine-1-carboxamide;
(6S)-6-[2-chloro-6-fluoro-3-(5-fluoro-2-methoxypyridine-3-sulfonamido)phenyl]-N-methyl-5H,6H,7H,8H-imidazo[1,5-a]pyridine-1-carboxamide;
methyl 2-[6-[2,6-difluoro-3-(5-fluoro-2-methoxypyridine-3-sulfonamido)phenyl]imidazo[1,5-a]pyrazin-1-yl]-3H-1,3-benzodiazole-5-carboxylate;
(6S)-6-[3-(5-chloro-2-methoxypyridine-3-sulfonamido)-2,6-difluorophenyl]-N-methyl-5H,6H,8H-imidazo[4,3-c][1,4]oxazine-1-carboxamide;
(6R)-6-[3-(5-chloro-2-methoxypyridine-3-sulfonamido)-2,6-difluorophenyl]-N-methyl-5H,6H,8H-imidazo[4,3-c][1,4]oxazine-1-carboxamide;
(6R)-6-[6-chloro-2-fluoro-3-(5-fluoro-2-methoxypyridine-3-sulfonamido)phenyl]-N-methyl-5H,6H,7H,8H-imidazo[1,5-a]pyridine-1-carboxamide;
(6S)-6-[6-chloro-2-fluoro-3-(5-fluoro-2-methoxypyridine-3-sulfonamido)phenyl]-N-methyl-5H,6H,7H,8H-imidazo[1,5-a]pyridine-1-carboxamide;
N-[2,4-difluoro-3-[1-(4-phenyl-1H-imidazol-2-yl)imidazo[1,5-a]pyrazin-6-yl]phenyl]-5-fluoro-2-methoxypyridine-3-sulfonamide;
6-[3-(5-chloro-2-methoxypyridine-3-sulfonamido)-2,6-difluorophenyl]-N-isopropylimidazo[1,5-a]pyrazine-1-carboxamide;
6-[3-(5-chloro-2-methoxypyridine-3-sulfonamido)-2,6-difluorophenyl]-N-(2-methylpropyl)imidazo[1,5-a]pyrazine-1-carboxamide;
6-[3-(5-chloro-2-methoxypyridine-3-sulfonamido)-2,6-difluorophenyl]-N-(2,2,2-trifluoroethyl)imidazo[1,5-a]pyrazine-1-carboxamide;
6-[3-(5-chloro-2-methoxypyridine-3-sulfonamido)-2,6-difluorophenyl]-5-fluoro-N-methylimidazo[1,5-a]pyrazine-1-carboxamide;
6-[3-(5-chloro-2-methoxypyridine-3-sulfonamido)-2,6-difluorophenyl]-N-(2-hydroxyethyl)imidazo[1,5-a]pyrazine-1-carboxamide;
6-[3-(5-chloro-2-methoxypyridine-3-sulfonamido)-2,6-difluorophenyl]-N-(1-methylpyrazol-4-yl)imidazo[1,5-a]pyrazine-1-carboxamide;
6-[3-(5-chloro-2-methoxypyridine-3-sulfonamido)-2,6-difluorophenyl]-N-(oxan-4-ylmethyl)imidazo[1,5-a]pyrazine-1-carboxamide;
6-[3-(5-chloro-2-methoxypyridine-3-sulfonamido)-2,6-difluorophenyl]-N-(2-methanesulfonylethyl)imidazo[1,5-a]pyrazine-1-carboxamide;
6-[3-(5-chloro-2-methoxypyridine-3-sulfonamido)-2,6-difluorophenyl]-N-ethylimidazo[1,5-a]pyrazine-1-carboxamide;
6-[3-(5-chloro-2-methoxypyridine-3-sulfonamido)-2,6-difluorophenyl]-N-(cyclopropylmethyl)imidazo[1,5-a]pyrazine-1-carboxamide;
6-[3-(5-chloro-2-ethoxypyridine-3-sulfonamido)-2,6-difluorophenyl]-N-methylimidazo[1,5-a]pyrazine-1-carboxamide;
6-[3-(5-chloro-2-isopropoxypyridine-3-sulfonamido)-2,6-difluorophenyl]-N-methylimidazo[1,5-a]pyrazine-1-carboxamide;
6-[3-(5-chloro-2-cyclopropoxypyridine-3-sulfonamido)-2,6-difluorophenyl]-N-methylimidazo[1,5-a]pyrazine-1-carboxamide;
6-[2,6-difluoro-3-(5-fluoro-2-methoxypyridine-3-sulfonamido)phenyl]-5-fluoro-N-methylimidazo[1,5-a]pyrazine-1-carboxamide;
6-[3-(5-chloro-2-methoxypyridine-3-sulfonamido)-2,6-difluorophenyl]imidazo[1,5-a]pyrazine-1-carboxamide;
6-[3-(5-chloro-2-methoxypyridine-3-sulfonamido)-2,6-difluorophenyl]-N-[3-(dimethylamino)propyl]imidazo[1,5-a]pyrazine-1-carboxamide;
(R)-6-(3-((5-chloro-2-methoxypyridine)-3-sulfonamido)-2,6-difluorophenyl)-N-(1-methylpyrrolidin-3-yl)imidazo[1,5-a]pyrazine-1-carboxamide;
(S)-6-(3-((5-chloro-2-methoxypyridine)-3-sulfonamido)-2,6-difluorophenyl)-N-(1-methylpyrrolidin-3-yl)imidazo[1,5-a]pyrazine-1-carboxamide;
6-[3-(5-chloro-2-methoxypyridine-3-sulfonamido)-2,6-difluorophenyl]-N-(1,2-oxazol-4-yl)imidazo[1,5-a]pyrazine-1-carboxamide;
6-[3-(5-chloro-2-methoxypyridine-3-sulfonamido)-2,6-difluorophenyl]-N-[2-(morpholin-4-yl)ethyl]imidazo[1,5-a]pyrazine-1-carboxamide;
6-[3-[5-chloro-2-(2-methylpropoxy)pyridine-3-sulfonamido]-2,6-difluorophenyl]-N-methylimidazo[1,5-a]pyrazine-1-carboxamide;
N-[3-[1-(5-cyclopropyl-3H-imidazol-4-yl)imidazo[1,5-a]pyrazin-6-yl]-2,4-difluorophenyl]-5-fluoro-2-methoxypyridine-3-sulfonamide;
N-[3-[1-(4-benzyl-1H-imidazol-2-yl)imidazo[1,5-a]pyrazin-6-yl]-2,4-difluorophenyl]-5-fluoro-2-methoxypyridine-3-sulfonamide;
6-[3-(5-chloro-2-methoxypyridine-3-sulfonamido)-2,6-difluorophenyl]-N-[2-(dimethylamino)ethyl]imidazo[1,5-a]pyrazine-1-carboxamide;
6-[3-(5-chloro-2-methoxypyridine-3-sulfonamido)-2,6-difluorophenyl]-N-[2-(2-oxopyrrolidin-1-yl)ethyl]imidazo[1,5-a]pyrazine-1-carboxamide;
6-[3-(5-chloro-2-methoxypyridine-3-sulfonamido)-2,6-difluorophenyl]-N-[4-[(dimethylamino)methyl]phenyl]imidazo[1,5-a]pyrazine-1-carboxamide;
6-[3-[5-chloro-2-(2,2,2-trifluoroethoxy)pyridine-3-sulfonamido]-2,6-difluorophenyl]-N-methylimidazo[1,5-a]pyrazine-1-carboxamide;
6-[3-[2-(benzyloxy)-5-chloropyridine-3-sulfonamido]-2,6-difluorophenyl]-N-methylimidazo[1,5-a]pyrazine-1-carboxamide;
6-[2,6-difluoro-3-(5-fluoro-2-methylpyridine-3-sulfonamido)phenyl]-5-fluoro-N-methylimidazo[1,5-a]pyrazine-1-carboxamide;
N-(2,4-difluoro-3-[1-[5-(4-methylpiperazin-1-yl)-3H-1,3-benzodiazol-2-yl]imidazo[1,5-a]pyridin-6-yl]phenyl)-5-fluoro-2-methoxypyridine-3-sulfonamide; and
6-[3-(5-chloro-2-methylpyridine-3-sulfonamido)-2,6-difluorophenyl]-5-fluoro-N-methylimidazo[1,5-a]pyrazine-1-carboxamide,
or a pharmaceutically acceptable salt thereof.

37. A pharmaceutical composition comprising a compound of claim 2 and a pharmaceutically acceptable carrier.

38. A method of treating cancer in a subject in need thereof, comprising administering to the subject a therapeutically effective amount of a compound of claim 2.

39.-45. (canceled)
